SEQUENCE LISTING (423 sequences)

SEQ ID NO: 1 - protein, 6 aa, synthetic construct
MLKKIE

SEQ ID NO: 2 - protein, 16 aa, synthetic construct
HMVCLAYRGRPVCFAL

SEQ ID NO: 3 - protein, 16 aa, synthetic construct
HMVCLSYRGRPVCFSL

SEQ ID NO: 4 - protein, 16 aa, synthetic construct
HQVCLSYRGRPVCFST

SEQ ID NO: 5 - protein, 16 aa, synthetic construct
QMRCLSYKGRRVCLWL

SEQ ID NO: 6 - protein, 16 aa, synthetic construct
KRLCLQYKGSKVCFRL

SEQ ID NO: 7 - protein, 16 aa, synthetic construct
RMRCLTYRGRRVCLEL

SEQ ID NO: 8 - protein, 16 aa, synthetic construct
SMRCLQYRGSRVCLTL

SEQ ID NO: 9 - protein, 16 aa, synthetic construct
HLRCLRYKGTRVCFSL

SEQ ID NO: 10 - protein, 16 aa, synthetic construct
HVRCLSYKGREVCVQL

SEQ ID NO: 11 - protein, 16 aa, synthetic construct
PRMCLFIYKGRRVCIP

SEQ ID NO: 12 - protein, 16 aa, synthetic construct
HMRCLHYKGRRVCFLL

SEQ ID NO: 13 - protein, 16 aa, synthetic construct
HKRCLHYRGRMVCFLI

SEQ ID NO: 14 - protein, 16 aa, synthetic construct
QKRCLKYKGSRVCFFL

SEQ ID NO: 15 - protein, 16 aa, synthetic construct
HVRCLRYRGKNVCFLL

SEQ ID NO: 16 - protein, 16 aa, synthetic construct
SDVCLRYRGRPVCFQV

SEQ ID NO: 17 - protein, 16 aa, synthetic construct
RDVCLRYRGRPVCFQV

SEQ ID NO: 18 - protein, 16 aa, synthetic construct
HDVCLRYRGRPVCFQV

SEQ ID NO: 19 - protein, 16 aa, synthetic construct
SMVCLRYRGRPVCFQV

SEQ ID NO: 20 - protein, 16 aa, synthetic construct
SAVCLRYRGRPVCFQV

SEQ ID NO: 21 - protein, 16 aa, synthetic construct
SDVCLNYRGRPVCFQV

SEQ ID NO: 22 - protein, 16 aa, synthetic construct
SDVCLHYRGRPVCFQV

SEQ ID NO: 23 - protein, 16 aa, synthetic construct
SDVCLAYRGRPVCFQV

SEQ ID NO: 24 - protein, 16 aa, synthetic construct
SDVCLRYRGRPVCFAV

SEQ ID NO: 25 - protein, 16 aa, synthetic construct
SDVCLRYRGRPVCFQL

SEQ ID NO: 26 - protein, 16 aa, synthetic construct
SDVCLRYRGRPVCFQA

SEQ ID NO: 27 - protein, 14 aa, synthetic construct
HMVCLSYRGRPVCF

SEQ ID NO: 28 - protein, 15 aa, synthetic construct
HMVCLSYRGRPVCFS

SEQ ID NO: 29 - protein, 16 aa, synthetic construct
HQVCLSYRGQPVCFSL

SEQ ID NO: 30 - protein, 16 aa, synthetic construct
HQVCLSYRGRPTCFSL

SEQ ID NO: 31 - protein, 16 aa, synthetic construct
HQVCLSYRGRPVCYSL

SEQ ID NO: 32 - protein, 16 aa, synthetic construct
HQVCLSYRGQPVCFST

SEQ ID NO: 33 - protein, 16 aa, synthetic construct
HQVCLSYRGRPTCFST

SEQ ID NO: 34 - protein, 16 aa, synthetic construct
HQVCLSYRGQPTCFST

SEQ ID NO: 35 - protein, 16 aa, synthetic construct
  CROSSLNK 3..7: note = Tyr-Lys
  CROSSLNK 7..12: note = Lys-Lys
  CROSSLNK 11..16: note = Lys-Tyr
RTYRTYKRTYKKGRTY

SEQ ID NO: 36 - protein, 16 aa, synthetic construct
  SITE 1..16: note = D-amino acids
  CROSSLNK 3..7: note = Tyr-Lys
  CROSSLNK 11..16: note = Lys-Tyr
  CROSSLNK 7..12: note = Lys-Lys
RTYRTYKRTYKKGRTY

SEQ ID NO: 37 - protein, 16 aa, synthetic construct
  SITE 1..16: note = D-Amino Acids
  CROSSLNK 3..7: note = Tyr-Lys
  CROSSLNK 11..16: note = Lys-Tyr
  CROSSLNK 7..12: note = Lys-Lys
  MOD_RES 1: note = Phenylacetic acid
  MOD_RES 4: note = Phenylacetic acid
  MOD_RES 8: note = Phenylacetic acid
  MOD_RES 14: note = Phenylacetic acid
RTYRTYKRTYKKGRTY

SEQ ID NO: 38 - protein, 10 aa, synthetic construct
TWKTSRISIF

SEQ ID NO: 39 - protein, 10 aa, synthetic construct
FGRLVSSIRY

SEQ ID NO: 40 - protein, 13 aa, synthetic construct
  CROSSLNK 2..12: note = Cys-Cys
DCAWHLGELVWCT

SEQ ID NO: 41 - protein, 12 aa, synthetic construct
DSAWHLGELWST

SEQ ID NO: 42 - protein, 13 aa, synthetic construct
DCHKRSFWADNCT

SEQ ID NO: 43 - protein, 13 aa, synthetic construct
DCRTQFRPNQTCT

SEQ ID NO: 44 - protein, 13 aa, synthetic construct
DCQLCDFWRTRCT

SEQ ID NO: 45 - protein, 13 aa, synthetic construct
DCFEDFNEQRTCT

SEQ ID NO: 46 - protein, 13 aa, synthetic construct
DCLAKFLKGKDCT

SEQ ID NO: 47 - protein, 13 aa, synthetic construct
DCWHRRTHKTFCT

SEQ ID NO: 48 - protein, 11 aa, synthetic construct
DCRTIQTRSCT

SEQ ID NO: 49 - protein, 13 aa, synthetic construct
DCIKLAQLHSVCT

SEQ ID NO: 50 - protein, 13 aa, synthetic construct
DCWRHRNATEWCT

SEQ ID NO: 51 - protein, 13 aa, synthetic construct
DCQNWIKDVHKCT

SEQ ID NO: 52 - protein, 13 aa, synthetic construct
DCAWHLGELVWCT

SEQ ID NO: 53 - protein, 13 aa, synthetic construct
DCAFHLGELVWCT

SEQ ID NO: 54 - protein, 13 aa, synthetic construct
DCAYHLGELVWCT

SEQ ID NO: 55 - protein, 11 aa, synthetic construct
  CROSSLNK 1..11: note = Pro-Pro
  SITE 11: note = D-Phenylalanine
PAWHLGELVWP

SEQ ID NO: 56 - protein, 15 aa, synthetic construct
  CROSSLNK 1..15: note = Pro-Pro
  CROSSLNK 3..13: note = Cys-Cys
  SITE 15: note = D-Amino Acid
PDCAWHLGELVWCTP

SEQ ID NO: 57 - protein, 15 aa, synthetic construct
  CROSSLNK 3..13: note = Cys-Cys
  CROSSLNK 1..15: note = Cys-Cys
CDCAWHLGELVWCTC

SEQ ID NO: 58 - protein, 12 aa, synthetic construct
EPIHRSTLTALL

SEQ ID NO: 59 - protein, 4 aa, synthetic construct
APAR

SEQ ID NO: 60 - protein, 6 aa, synthetic construct
  REPEAT 1..4: note = repeated twice
  CROSSLNK 4..5: note = His-Lys
  CROSSLNK 1: note = Cys-Cys
CFHHKG

SEQ ID NO: 61 - protein, 6 aa, synthetic construct
HWRGWV

SEQ ID NO: 62 - protein, 6 aa, synthetic construct
HYFKFD

SEQ ID NO: 63 - protein, 6 aa, synthetic construct
HFRRHL

SEQ ID NO: 64 - protein, 6 aa, synthetic construct
  NON_STD 3: note = Citrulline
HWXGWV

SEQ ID NO: 65 - protein, 6 aa, synthetic construct
  NON_STD 3: note = Citrulline
  MOD_RES 2: note = MeTrp
  MOD_RES 5: note = MeTrp
HWXGWV

SEQ ID NO: 66 - protein, 4 aa, synthetic construct
  REPEAT 1: note = repeated twice
  CROSSLNK 1..2: note = Asp-Ala
DAAG

SEQ ID NO: 67 - protein, 4 aa, synthetic construct
DAAG

SEQ ID NO: 68 - protein, 9 aa, synthetic construct
  CROSSLNK 1..9: note = N(alpha)acetyl Linker (Ser-Glu)
  CROSSLNK 8..9: note = Lactic acid Linker (Lys-Glu)
SARWHYFKE

SEQ ID NO: 69 - protein, 9 aa, synthetic construct
  CROSSLNK 1..9: note = N(alpha)acetyl Linker (Dpr-Glu)
  CROSSLNK 8..9: note = Lactic acid Linker (Lys-Glu)
  MOD_RES 1: note = Dpr
XARWHYFKE

SEQ ID NO: 70 - protein, 7 aa, synthetic construct
  CROSSLNK 1..7: note = Link M-Lys
  NON_STD 1: note = Link M
XWFRHYK

SEQ ID NO: 71 - protein, 8 aa, synthetic construct
NKFRGKYK

SEQ ID NO: 72 - protein, 8 aa, synthetic construct
NARKFYKG

SEQ ID NO: 73 - protein, 8 aa, synthetic construct
FYWHCLDE

SEQ ID NO: 74 - protein, 8 aa, synthetic construct
FYCHWALE

SEQ ID NO: 75 - protein, 8 aa, synthetic construct
FYCHTIDE

SEQ ID NO: 76 - protein, 16 aa, synthetic construct
FYWHCLDEFYCHTIDE

SEQ ID NO: 77 - protein, 4 aa, synthetic construct
RRGW

SEQ ID NO: 78 - protein, 7 aa, synthetic construct
KHRFNKD

SEQ ID NO: 79 - protein, 7 aa, synthetic construct
CPSTHWK

SEQ ID NO: 80 - protein, 7 aa, synthetic construct
NVQYFAV

SEQ ID NO: 81 - protein, 7 aa, synthetic construct
ASHTQKS

SEQ ID NO: 82 - protein, 7 aa, synthetic construct
QPQMSHM

SEQ ID NO: 83 - protein, 7 aa, synthetic construct
TNIESLK

SEQ ID NO: 84 - protein, 7 aa, synthetic construct
NCHKCWN

SEQ ID NO: 85 - protein, 7 aa, synthetic construct
SHLSKNF

SEQ ID NO: 86 - protein, 8 aa, synthetic construct
FDLLEHFY

SEQ ID NO: 87 - protein, 9 aa, synthetic construct
DLLHHFDYF

SEQ ID NO: 88 - protein, 13 aa, synthetic construct
GIFVIQSESLKKC

SEQ ID NO: 89 - protein, 9 aa, synthetic construct
SVLTLENCK

SEQ ID NO: 90 - protein, 8 aa, synthetic construct
SVLTLENC

SEQ ID NO: 91 - protein, 9 aa, synthetic construct
SVLTLDNCK

SEQ ID NO: 92 - protein, 8 aa, synthetic construct
SVLTEENC

SEQ ID NO: 93 - protein, 8 aa, synthetic construct
SVLTEENS

SEQ ID NO: 94 - protein, 8 aa, synthetic construct
SVLTDENC

SEQ ID NO: 95 - protein, 8 aa, synthetic construct
SVLTDENS

SEQ ID NO: 96 - protein, 9 aa, unidentified
PIQSESLKK

SEQ ID NO: 97 - protein, 9 aa, synthetic construct
VIDSESLKK

SEQ ID NO: 98 - protein, 9 aa, synthetic construct
PIDSESLKK

SEQ ID NO: 99 - protein, 9 aa, synthetic construct
VIQSESLKK

SEQ ID NO: 100 - protein, 21 aa, synthetic construct
PIESESPEGKPEGSVLTEENC

SEQ ID NO: 101 - protein, 21 aa, synthetic construct
VIQSESPEGKPEGSVLTLENC

SEQ ID NO: 102 - protein, 21 aa, synthetic construct
VIQSESPEGKPEGSVLTEENC

SEQ ID NO: 103 - protein, 21 aa, synthetic construct
PIDDESPEGKPEGSVLTLENC

SEQ ID NO: 104 - protein, 21 aa, synthetic construct
PIDDESPEGKPEGSVLTEENC

SEQ ID NO: 105 - protein, 19 aa, synthetic construct
VIQSESLKKCKSVLTLENC

SEQ ID NO: 106 - protein, 19 aa, synthetic construct
PIQSESLKKCKSVLTLENC

SEQ ID NO: 107 - protein, 19 aa, synthetic construct
VIESESLKKCKSVLTLENC

SEQ ID NO: 108 - protein, 19 aa, synthetic construct
VIDSESLKKCKSVLTLENC

SEQ ID NO: 109 - protein, 19 aa, synthetic construct
PIESESLKKCKSVLTLENC

SEQ ID NO: 110 - protein, 19 aa, synthetic construct
VIQSESLKKCIQAGKLENC

SEQ ID NO: 111 - protein, 19 aa, synthetic construct
PIQSESLKKCIQAGKLENC

SEQ ID NO: 112 - protein, 19 aa, synthetic construct
VIESESLKKCIQAGKLENC

SEQ ID NO: 113 - protein, 19 aa, synthetic construct
VIDSESLKKCIQAGKLENC

SEQ ID NO: 114 - protein, 19 aa, synthetic construct
PIESESLKKCIQAGKLENC

SEQ ID NO: 115 - protein, 19 aa, synthetic construct
PIQSESLKKCKSVLTLENK

SEQ ID NO: 116 - protein, 19 aa, synthetic construct
VIESESLKKCKSVLTLENK

SEQ ID NO: 117 - protein, 19 aa, synthetic construct
VIDSESLKKCKSVLTLENK

SEQ ID NO: 118 - protein, 19 aa, synthetic construct
PIESESLKKCKSVLTLENK

SEQ ID NO: 119 - protein, 19 aa, synthetic construct
VIQSESLKKCIQAGKLENK

SEQ ID NO: 120 - protein, 19 aa, synthetic construct
PIQSESLKKCIQAGKLENK

SEQ ID NO: 121 - protein, 19 aa, synthetic construct
VIESESLKKCIQAGKLENK

SEQ ID NO: 122 - protein, 19 aa, synthetic construct
VIDSESLKKCIQAGKLENK

SEQ ID NO: 123 - protein, 19 aa, synthetic construct
PIESESLKKCIQAGKLENK

SEQ ID NO: 124 - protein, 16 aa, synthetic construct
PIESESGSVLTLENCK

SEQ ID NO: 125 - protein, 17 aa, synthetic construct
PIESESGGSVLTLENCK

SEQ ID NO: 126 - protein, 18 aa, synthetic construct
PIESESGGGSVLTLENCK

SEQ ID NO: 127 - protein, 19 aa, synthetic construct
PIESESGGGGSVLTLENCK

SEQ ID NO: 128 - protein, 20 aa, synthetic construct
PIESESGGGGGSVLTLENCK

SEQ ID NO: 129 - protein, 16 aa, synthetic construct
VIQSESGSVLTLENCK

SEQ ID NO: 130 - protein, 17 aa, synthetic construct
VIQSESGGSVLTLENCK

SEQ ID NO: 131 - protein, 18 aa, synthetic construct
VIQSESGGGSVLTLENCK

SEQ ID NO: 132 - protein, 19 aa, synthetic construct
VIQSESGGGGSVLTLENCK

SEQ ID NO: 133 - protein, 20 aa, synthetic construct
VIQSESGGGGGSVLTLENCK

SEQ ID NO: 134 - protein, 28 aa, synthetic construct
KGCFVIQSESLKKSIQAGKSVLTLENCK

SEQ ID NO: 135 - protein, 21 aa, synthetic construct
LKKCIQAGKSVLTLENCKQAN

SEQ ID NO: 136 - protein, 22 aa, synthetic construct
WQDKGIFVIQSESLKKCIQAGK

SEQ ID NO: 137 - protein, 28 aa, synthetic construct
KGIFVIQSESLKKCIQAGKSVLTLENCK

SEQ ID NO: 138 - protein, 13 aa, synthetic construct
GIFVIQSESLKKC

SEQ ID NO: 139 - protein, 10 aa, synthetic construct
WSVLTLENCK

SEQ ID NO: 140 - protein, 31 aa, synthetic construct
WQDKGIFVIQSESLKKCIQAGKSVLTLENCK

SEQ ID NO: 141 - protein, 9 aa, synthetic construct
YDWIPSSAW

SEQ ID NO: 142 - protein, 9 aa, synthetic construct
AGAIWQRDW

SEQ ID NO: 143 - protein, 9 aa, synthetic construct
AGAIWQKDW

SEQ ID NO: 144 - protein, 8 aa, synthetic construct
VIQSESLK

SEQ ID NO: 145 - protein, 8 aa, synthetic construct
PIQSESLK

SEQ ID NO: 146 - protein, 8 aa, synthetic construct
PIESESLK

SEQ ID NO: 147 - protein, 9 aa, synthetic construct
SVLTEENCK

SEQ ID NO: 148 - protein, 8 aa, synthetic construct
  SITE 5: note = Any Naturally-Occurring Amino Acid
  SITE 8: note = Any Naturally-Occurring Amino Acid
SVLTXENX

SEQ ID NO: 149-150 - skipped (2 entries)

SEQ ID NO: 151 - protein, 9 aa, synthetic construct
  SITE 5: note = Any Naturally-Occurring Amino Acid
SVLTXENCK

SEQ ID NO: 152 - protein, 8 aa, synthetic construct
  SITE 1: note = Any Naturally-Occurring Amino Acid
  SITE 3: note = Any Naturally-Occurring Amino Acid
  SITE 4: note = Any Naturally-Occurring Amino Acid
  SITE 6: note = Any Naturally-Occurring Amino Acid
XIXXEXLK

SEQ ID NO: 153 - protein, 14 aa, synthetic construct
  MOD_RES 5: note = MeTrp
  MOD_RES 14: note = MeIle
  DISULFID 3..13: note = Cys-Cys
  SITE 1: note = D-Amino Acid
  MOD_RES 9: note = MeGly
YICVWQDWGAHRCI

SEQ ID NO: 154 - protein, 26 aa, synthetic construct
  DISULFID 2..12: note = Cys-Cys
ICVVQDWGHHRCTAGMANLTSHASAI

SEQ ID NO: 155 - protein, 13 aa, synthetic construct
  DISULFID 2..12: note = Cys-Cys
ICVVQDWGHHRCT

SEQ ID NO: 156 - protein, 11 aa, synthetic construct
  DISULFID 1..11: note = Cys-Cys
CVVQDWGHHAC

SEQ ID NO: 157 - protein, 13 aa, synthetic construct
  MOD_RES 1: note = AcIle
  DISULFID 2..12: note = Cys-Cys
ICVVQDWGHHRCT

SEQ ID NO: 158 - protein, 12 aa, synthetic construct
  DISULFID 1..11: note = Cys-Cys
CVVQDWGHHRCT

SEQ ID NO: 159 - protein, 11 aa, synthetic construct
  DISULFID 1..11: note = Cys-Cys
CVVQDWGHHRC

SEQ ID NO: 160 - protein, 13 aa, synthetic construct
  MOD_RES 1: note = AcIle
  DISULFID 2..12: note = Cys-Cys
ICVVGDWGHHRCT

SEQ ID NO: 161 - protein, 13 aa, synthetic construct
  MOD_RES 1: note = AcIle
  DISULFID 2..12: note = Cys-Cys
ICVVQPWGHHRCT

SEQ ID NO: 162 - protein, 17 aa, synthetic construct
  DISULFID 6..16: note = Cys-Cys
KYSSICVVQDWGHHRCT

SEQ ID NO: 163 - protein, 27 aa, synthetic construct
  MOD_RES 1: note = AcIle
  DISULFID 2..12: note = Cys-Cys
ICVVQDWGHHRCTAGHMANLTSHASAK

SEQ ID NO: 164 - protein, 13 aa, synthetic construct
  MOD_RES 1: note = AcIle
  MOD_RES 4: note = MeTrp
ICVWQDWGAHRCT

SEQ ID NO: 165 - protein, 13 aa, synthetic construct
  MOD_RES 1: note = vIle
  MOD_RES 8: note = MeGly
  MOD_RES 13: note = MeIle
  MOD_RES 4: note = MeTrp
ICVWQDWGAHRCI

SEQ ID NO: 166 - protein, 13 aa, synthetic construct
  MOD_RES 1: note = vIle
  MOD_RES 4: note = MeTrp
  MOD_RES 8: note = MeGly
  MOD_RES 13: note = MeIle
ICVWQDWGAHRCI

SEQ ID NO: 167 - protein, 13 aa, synthetic construct
  DISULFID 2..12: note = Cys-Cys
  MOD_RES 1: note = AcIle
ICVWQDWGAHRCT

SEQ ID NO: 168 - protein, 13 aa, synthetic construct
  DISULFID 2..12: note = Cys-Cys
  MOD_RES 1: note = MeIle
  MOD_RES 4: note = MeTrp
  MOD_RES 8: note = MeGly
ICVWQDWGAHRCI

SEQ ID NO: 169 - protein, 13 aa, synthetic construct
  MOD_RES 1: note = AcIle
  MOD_RES 4: note = MeTrp
  MOD_RES 8: note = MeGly
  MOD_RES 13: note = MeIle
  DISULFID 2..12: note = Cys-Cys
ICVWQDWGAHRCI

SEQ ID NO: 170 - protein, 13 aa, synthetic construct
  DISULFID 2..12: note = Cys-Cys
  MOD_RES 1: note = AcIle
  MOD_RES 4: note = MeTrp
ICVWQDWGAHRCT

SEQ ID NO: 171 - protein, 13 aa, synthetic construct
  DISULFID 2..13: note = Cys-Cys
  MOD_RES 1: note = AcXaa
XCVXQDWGXXXCT

SEQ ID NO: 172 - protein, 13 aa, synthetic construct
  DISULFID 2..12: note = Cys-Cys
  MOD_RES 1: note = AcIle
ICVVQDWGHHRCT

SEQ ID NO: 173 - protein, 13 aa, synthetic construct
  MOD_RES 1: note = AcIle
  DISULFID 2..12: note = Cys-Cys
ICVVQDWGAHRCT

SEQ ID NO: 174 - protein, 13 aa, synthetic construct
  MOD_RES 1: note = AcIle
  DISULFID 2..12: note = Cys-Cys
ICVTQDWGHHRCT

SEQ ID NO: 175 - protein, 13 aa, synthetic construct
  MOD_RES 1: note = AcIle
  DISULFID 2..12: note = Cys-Cys
ICVSQDWGHHRCT

SEQ ID NO: 176 - protein, 13 aa, synthetic construct
  MOD_RES 1: note = AcIle
  DISULFID 2..12: note = Cys-Cys
ICVHQDWGHHRCT

SEQ ID NO: 177 - protein, 13 aa, synthetic construct
  MOD_RES 1: note = AcIle
  DISULFID 2..12: note = Cys-Cys
ICVFQDWGHHRCT

SEQ ID NO: 178 - protein, 13 aa, unidentified
  MOD_RES 1: note = AcIle
  DISULFID 2..12: note = Cys-Cys
ICVYQDWGAHRCT

SEQ ID NO: 179 - protein, 13 aa, synthetic construct
  MOD_RES 1: note = AcIle
  DISULFID 2..12: note = Cys-Cys
ICVWQDWGWHRCT

SEQ ID NO: 180 - protein, 13 aa, synthetic construct
  MOD_RES 1: note = AcIle
  DISULFID 2..12: note = Cys-Cys
ICVWQDWGHHRCT

SEQ ID NO: 181 - protein, 13 aa, synthetic construct
  MOD_RES 1: note = AcIle
  DISULFID 2..12: note = Cys-Cys
ICVWQDWGAHRCT

SEQ ID NO: 182 - protein, 13 aa, synthetic construct
  MOD_RES 1: note = AcIle
  DISULFID 2..12: note = Cys-Cys
ICVWQDWGAHRCT

SEQ ID NO: 183 - protein, 13 aa, synthetic construct
  MOD_RES 1: note = AcIle
  DISULFID 2..12: note = Cys-Cys
  SITE 10: note = D-Amino Acid
ICVWQDWGAHRCT

SEQ ID NO: 184 - protein, 13 aa, synthetic construct
  MOD_RES 1: note = AcIle
  DISULFID 2..12: note = Cys-Cys
  SITE 9: note = D-Amino Acid
ICVWQDWGAHRCT

SEQ ID NO: 185 - protein, 13 aa, synthetic construct
  MOD_RES 1: note = AcIle
  SITE 1: note = D-Amino Acid
  DISULFID 2..12: note = Cys-Cys
ICVWQDWGAHRCT

SEQ ID NO: 186 - protein, 13 aa, synthetic construct
  DISULFID 2..12: note = Cys-Cys
  MOD_RES 1: note = AcIle
  SITE 13: note = D-Amino Acid
ICVWQDWGAHRCT

SEQ ID NO: 187 - protein, 13 aa, synthetic construct
  DISULFID 2..12: note = Cys-Cys
  MOD_RES 1: note = Ac-Ile
ICVWQDWGAHRCT

SEQ ID NO: 188 - protein, 13 aa, synthetic construct
  DISULFID 2..12: note = Cys-Cys
WCVWQDWGTNRCW

SEQ ID NO: 189 - protein, 13 aa, synthetic construct
  MOD_RES 1: note = Ac-Asp
  DISULFID 2..12: note = Cys-Cys
DCVWQDWGTNKCW

SEQ ID NO: 190 - protein, 13 aa, synthetic construct
  DISULFID 2..12: note = Cys-Cys
QCVWQDWGQNQCW

SEQ ID NO: 191 - protein, 13 aa, synthetic construct
  DISULFID 2..12: note = Cys-Cys
  MOD_RES 1: note = AcIle
ICVWQDWGAHRCW

SEQ ID NO: 192 - protein, 13 aa, synthetic construct
  MOD_RES 1: note = AcTrp
  DISULFID 2..12: note = Cys-Cys
WCVWQDWGAHRCT

SEQ ID NO: 193 - protein, 13 aa, synthetic construct
  MOD_RES 1: note = AcTrp
  DISULFID 2..12: note = Cys-Cys
WCVWQDWGAHRCW

SEQ ID NO: 194 - protein, 13 aa, synthetic construct
  SITE 12: note = Homocysteine
  MOD_RES 1: note = AcIle
  CROSSLNK 2..12: note = Ala-Hcy
IAVWQDWGAHRXT

SEQ ID NO: 195 - protein, 13 aa, synthetic construct
  MOD_RES 1: note = AcIle
  DISULFID 2..12: note = Cys-Cys
  MOD_RES 13: note = MeIle
ICVWQDWGAHRCI

SEQ ID NO: 196 - protein, 13 aa, synthetic construct
  MOD_RES 1: note = AcIle
  DISULFID 2..12: note = Ala-Hcy
  MOD_RES 13: note = MeIle
  SITE 12: note = Homocysteine
IAVWQDWGAHRXI

SEQ ID NO: 197 - protein, 13 aa, synthetic construct
  MOD_RES 1: note = AcIle
  MOD_RES 4: note = 5fTrp
ICVWQDWGAHRCT

SEQ ID NO: 198 - protein, 13 aa, synthetic construct
  MOD_RES 1: note = AcIle
  MOD_RES 4: note = m5Trp
ICVWQDWGAHRCT

SEQ ID NO: 199 - protein, 13 aa, synthetic construct
  SITE 4: note = Naphthylalanine
  MOD_RES 1: note = AcIle
ICVXQDWGAHRCT

SEQ ID NO: 200 - protein, 13 aa, synthetic construct
  MOD_RES 1: note = AcIle
  MOD_RES 7: note = 5fTrp
ICVWQDWGAHRCT

SEQ ID NO: 201 - protein, 13 aa, synthetic construct
  MOD_RES 1: note = AcIle
  MOD_RES 7: note = m5Trp
ICVWQDWGAHRCT

SEQ ID NO: 202 - protein, 13 aa, synthetic construct
  MOD_RES 1: note = AcIle
  MOD_RES 7: note = MeTrp
ICVWQDWGAHRCT

SEQ ID NO: 203 - protein, 13 aa, synthetic construct
  MOD_RES 1: note = AcIle
ICVYQDWGAHRCT

SEQ ID NO: 204 - protein, 13 aa, synthetic construct
  MOD_RES 1: note = AcIle
ICVWQDWGAHRCT

SEQ ID NO: 205 - protein, 13 aa, synthetic construct
  MOD_RES 1: note = AcIle
ICVWQDWGAHRCT

SEQ ID NO: 206 - protein, 13 aa, synthetic construct
  MOD_RES 1: note = AcIle
  SITE 13: note = D-Amino Acid
ICVWQDWGAHRCT

SEQ ID NO: 207 - protein, 13 aa, synthetic construct
  MOD_RES 1: note = AcIle
  SITE 4: note = Naphthylalanine
ICVXQDWGAHRCT

SEQ ID NO: 208 - protein, 13 aa, synthetic construct
  MOD_RES 1: note = AcIle
  SITE 4: note = Naphthylalanine
ICVXQDWGAHRCT

SEQ ID NO: 209 - protein, 13 aa, synthetic construct
  MOD_RES 1: note = AcIle
  SITE 4: note = Naphthylalanine
ICVXQDWGAHRCT

SEQ ID NO: 210 - protein, 13 aa, synthetic construct
  MOD_RES 1: note = AcIle
  SITE 4: note = Lysoalanine
ICVXQDWGAHRCT

SEQ ID NO: 211 - protein, 13 aa, synthetic construct
  MOD_RES 1: note = AcIle
  SITE 4: note = Lysoalanine
ICVXQDWGAHRCT

SEQ ID NO: 212 - protein, 13 aa, synthetic construct
  MOD_RES 1: note = AcIle
  MOD_RES 5: note = htQ
ICVDQDWGAHRCT

SEQ ID NO: 213 - protein, 13 aa, synthetic construct
  MOD_RES 1: note = AcIle
  SITE 4: note = Boa
ICVXQDWGAHRCT

SEQ ID NO: 214 - protein, 13 aa, synthetic construct
  MOD_RES 1: note = AcIle
  SITE 4: note = 4-Benzoylphenylalanine
ICVXQDWGAHRCT

SEQ ID NO: 215 - protein, 13 aa, synthetic construct
  MOD_RES 1: note = AcIle
  SITE 4: note = L-3-Benzothienylalanine
ICVXQDWGAHRCT

SEQ ID NO: 216 - protein, 13 aa, synthetic construct
  MOD_RES 1: note = AcIle
  SITE 4: note = L-3-Benzothienylalanine
ICVXQDWGAHRCT

SEQ ID NO: 217 - protein, 13 aa, synthetic construct
  MOD_RES 1: note = AcIle
  SITE 9: note = Abu
ICVWQDWGXHRCT

SEQ ID NO: 218 - protein, 16 aa, synthetic construct
  MOD_RES 1: note = H-Gly
GICVWQDWGAHRCTAN

SEQ ID NO: 219 - protein, 13 aa, synthetic construct
  MOD_RES 1: note = AcIle
  MOD_RES 4: note = 5fTrp
ICVWQDWGAHRCT

SEQ ID NO: 220 - protein, 13 aa, synthetic construct
  MOD_RES 1: note = AcIle
  MOD_RES 4: note = m5Trp
ICVWQDWGAHRCT

SEQ ID NO: 221 - protein, 13 aa, synthetic construct
  MOD_RES 1: note = AcIle
  MOD_RES 4: note = MeTrp
ICVWQDWGAHRCT

SEQ ID NO: 222 - protein, 13 aa, synthetic construct
  MOD_RES 1: note = AcIle
  MOD_RES 4: note = 5fTrp
ICVWQDWGAHRCT

SEQ ID NO: 223 - protein, 13 aa, synthetic construct
  MOD_RES 1: note = AcIle
  MOD_RES 4: note = 5fTrp
  MOD_RES 7: note = 5fTrp
ICVWQDWGAHRCT

SEQ ID NO: 224 - protein, 13 aa, synthetic construct
  MOD_RES 1: note = AcIle
  MOD_RES 4: note = m5Trp
  MOD_RES 7: note = 5fTrp
ICVWQDWGAHRCT

SEQ ID NO: 225 - protein, 13 aa, synthetic construct
  MOD_RES 1: note = AcIle
  MOD_RES 4: note = MeTrp
  MOD_RES 7: note = 5fTrp
ICVWQDWGAHRCT

SEQ ID NO: 226 - protein, 15 aa, synthetic construct
  MOD_RES 1: note = H-Gly
  MOD_RES 4: note = 6fTrp
  MOD_RES 7: note = 6fTrp
GICVWQDWGAHRCTN

SEQ ID NO: 227 - protein, 13 aa, synthetic construct
  MOD_RES 1: note = AcIle
  MOD_RES 4: note = 1-formylTrp
ICVWQDWGAHRCT

SEQ ID NO: 228 - protein, 13 aa, synthetic construct
  MOD_RES 1: note = AcIle
  MOD_RES 4: note = 1-methyoxyTrp
ICVWQDWGAHRCT

SEQ ID NO: 229 - protein, 15 aa, synthetic construct
  MOD_RES 1: note = H-Gly
  MOD_RES 5: note = 5fTrp
  MOD_RES 7: note = 5fTrp
GICVWQDWGAHRCTN

SEQ ID NO: 230 - protein, 7 aa, synthetic construct
  MOD_RES 1: note = AcAla
AEAKAKA

SEQ ID NO: 231 - protein, 15 aa, synthetic construct
IALILEPICCQERAA

SEQ ID NO: 232 - protein, 15 aa, synthetic construct
IALILEPICCQERAA

SEQ ID NO: 233 - protein, 15 aa, synthetic construct
IALILEPICCQERAA

SEQ ID NO: 234 - protein, 15 aa, synthetic construct
RALILEPICCQERAA

SEQ ID NO: 235 - protein, 15 aa, synthetic construct
IRLILEPICCQERAA

SEQ ID NO: 236 - protein, 15 aa, synthetic construct
IARILEPICCQERAA

SEQ ID NO: 237 - protein, 15 aa, synthetic construct
IALIREPICCQERAA

SEQ ID NO: 238 - protein, 15 aa, synthetic construct
IALILEPICCRERAA

SEQ ID NO: 239 - protein, 15 aa, synthetic construct
IALILEPICCQRRAA

SEQ ID NO: 240 - protein, 15 aa, synthetic construct
IELILEPICCQERAA

SEQ ID NO: 241 - protein, 15 aa, synthetic construct
IAEILEPICCQERAA

SEQ ID NO: 242 - protein, 15 aa, synthetic construct
IALILEPICCQEEAA

SEQ ID NO: 243 - protein, 15 aa, synthetic construct
IALILEPICCQEREA

SEQ ID NO: 244 - protein, 15 aa, synthetic construct
IALILEEICCQERAA

SEQ ID NO: 245 - protein, 15 aa, synthetic construct
IALILEPECCQERAA

SEQ ID NO: 246 - protein, 30 aa, synthetic construct
PAICQRATATLGTVGSNTSGTTAIEACILL

SEQ ID NO: 247 - protein, 15 aa, synthetic construct
  DISULFID 1..12: note = Cys-Cys
CEGPFGPRHDLTFCW

SEQ ID NO: 248 - protein, 15 aa, synthetic construct
  SITE 1: note = Xb
  DISULFID 1..12: note = Xb-Cys
XEGPFGPRHDLTFCW

SEQ ID NO: 249 - protein, 7 aa, synthetic construct
QYYPFSX

SEQ ID NO: 250 - protein, 7 aa, synthetic construct
NPFNLAR

SEQ ID NO: 251 - protein, 12 aa, synthetic construct
QLQDMTSSPFWL

SEQ ID NO: 252 - protein, 12 aa, synthetic construct
NPFVIGRWHPPH

SEQ ID NO: 253 - protein, 12 aa, synthetic construct
SLAKFLNPFLYR

SEQ ID NO: 254 - protein, 12 aa, synthetic construct
ASTPRFEPFQLD

SEQ ID NO: 255 - protein, 12 aa, synthetic construct
SLHSQPYSPFML

SEQ ID NO: 256 - protein, 12 aa, synthetic construct
NILSSWSSPFVF

SEQ ID NO: 257 - protein, 12 aa, synthetic construct
NLPSSWTNPFYL

SEQ ID NO: 258 - protein, 7 aa, synthetic construct
SPFMLHP

SEQ ID NO: 259 - protein, 7 aa, synthetic construct
PSPFMLT

SEQ ID NO: 260 - protein, 7 aa, synthetic construct
IGPFHLH

SEQ ID NO: 261 - protein, 7 aa, synthetic construct
TNPFMLN

SEQ ID NO: 262 - protein, 7 aa, synthetic construct
NTTFLYP

SEQ ID NO: 263 - protein, 7 aa, synthetic construct
SHYTQYL

SEQ ID NO: 264 - protein, 7 aa, synthetic construct
NHHPNYW

SEQ ID NO: 265 - protein, 7 aa, synthetic construct
VHYPLSW

SEQ ID NO: 266 - protein, 12 aa, synthetic construct
HHLKYSDTSPPI

SEQ ID NO: 267 - protein, 13 aa, synthetic construct
SHMHERWDTSPPI

SEQ ID NO: 268 - protein, 12 aa, synthetic construct
SHMHERWDTSYQ

SEQ ID NO: 269 - protein, 12 aa, synthetic construct
SHIHSNAAWRIT

SEQ ID NO: 270 - protein, 7 aa, synthetic construct
WHYPHWQ

SEQ ID NO: 271 - protein, 7 aa, synthetic construct
SHYLYTQ

SEQ ID NO: 272 - protein, 7 aa, synthetic construct
AHYSFTQ

SEQ ID NO: 273 - protein, 7 aa, synthetic construct
THYPTFY

SEQ ID NO: 274 - protein, 7 aa, synthetic construct
EHNTSFW

SEQ ID NO: 275 - protein, 7 aa, synthetic construct
NHYKLTW

SEQ ID NO: 276 - protein, 7 aa, synthetic construct
NHSPYFQ

SEQ ID NO: 277 - protein, 7 aa, synthetic construct
SHYQHYQ

SEQ ID NO: 278 - protein, 30 aa, synthetic construct
PAICQRATATLGTVGSNTSGTTEIEACILL

SEQ ID NO: 279 - protein, 10 aa, synthetic construct
WLGLGGGYGW

SEQ ID NO: 280 - protein, 15 aa, synthetic construct
FYGPFFLNDSLRGIW

SEQ ID NO: 281 - protein, 15 aa, synthetic construct
LRFLNPFSLDGSGFW

SEQ ID NO: 282 - protein, 15 aa, synthetic construct
HSPFCLGVLECFGLV

SEQ ID NO: 283 - protein, 15 aa, synthetic construct
TCGAFYLYHDPFICG

SEQ ID NO: 284 - protein, 15 aa, synthetic construct
MQHCLASHELYLPWC

SEQ ID NO: 285 - protein, 15 aa, synthetic construct
FFVFGSGDAFAFSDM

SEQ ID NO: 286 - protein, 15 aa, synthetic construct
PCVIIDTGSSRWCYL

SEQ ID NO: 287 - protein, 15 aa, synthetic construct
HSPFCLGVLECFGLV

SEQ ID NO: 288 - protein, 15 aa, synthetic construct
HAAFEPRGDVRHTLL

SEQ ID NO: 289 - protein, 12 aa, synthetic construct
CRWDGSWGEVRC

SEQ ID NO: 290 - protein, 12 aa, synthetic construct
CYWVGTWGEAVC

SEQ ID NO: 291 - protein, 16 aa, synthetic construct
RWFPCPNKEGCCSISV

SEQ ID NO: 292 - protein, 16 aa, synthetic construct
RSTYCNKNKDSCHIPE

SEQ ID NO: 293 - protein, 16 aa, synthetic construct
QPPQCIKDGGFVICRV

SEQ ID NO: 294 - protein, 16 aa, synthetic construct
KGKKCKPEEHPCNEPM

SEQ ID NO: 295 - protein, 16 aa, synthetic construct
NKMTCSDDGKLCWEHL

SEQ ID NO: 296 - protein, 14 aa, synthetic construct
PLGRPCPTCPLAPS

SEQ ID NO: 297 - protein, 14 aa, synthetic construct
QRMRPCPSCPLAPW

SEQ ID NO: 298 - protein, 14 aa, synthetic construct
WPSRPCPSCPEVPP

SEQ ID NO: 299 - protein, 14 aa, synthetic construct
SCTKDCPTCPLVPV

SEQ ID NO: 300 - protein, 15 aa, synthetic construct
IALILEPICCQERAA

SEQ ID NO: 301 - protein, 30 aa, synthetic construct; identical to SEQ ID NO: 278

SEQ ID NO: 302 - protein, 30 aa, synthetic construct
PAIAQRATATLGTVGSNTSGTTEIEACILL

SEQ ID NO: 303 - protein, 30 aa, synthetic construct
PAICQRATATLGTVGSNTSGTTEIEAAILL

SEQ ID NO: 304 - protein, 30 aa, synthetic construct; identical to SEQ ID NO: 246

SEQ ID NO: 305 - protein, 30 aa, synthetic construct
PAICQRATATLGTVGSNTSGTTEIAACILL

SEQ ID NO: 306 - protein, 15 aa, synthetic construct
PAICQRAEIEACILL

SEQ ID NO: 307 - protein, 15 aa, synthetic construct
PAICQRAEIEACILL

SEQ ID NO: 308 - protein, 15 aa, synthetic construct
PAIAQRAEIEAAILL

SEQ ID NO: 309 - protein, 15 aa, synthetic construct
IALILEPICCQERAA

SEQ ID NO: 310 - protein, 27 aa, synthetic construct
PAICQRATATLGTNTSGTTEIEACILL

SEQ ID NO: 311 - protein, 23 aa, synthetic construct
PAICQRATATLSGTTEIEACILL

SEQ ID NO: 312 - protein, 19 aa, synthetic construct
PAICQRATATTEIEACILL

SEQ ID NO: 313 - protein, 15 aa, synthetic construct
PAICQRAEIEACILL

SEQ ID NO: 314 - protein, 29 aa, synthetic construct
AICQRATATLGTVGSNTSGTTEIEACILL

SEQ ID NO: 315 - protein, 28 aa, synthetic construct
ICQRATATLGTVGSNTSGTTEIEACILL

SEQ ID NO: 316 - protein, 27 aa, synthetic construct
CQRATATLGTVGSNTSGTTEIEACILL

SEQ ID NO: 317 - protein, 29 aa, synthetic construct
PAICQRATATLGTVGSNTSGTTEIEACIL

SEQ ID NO: 318 - protein, 28 aa, synthetic construct
PAICQRATATLGTVGSNTSGTTEIEACI

SEQ ID NO: 319 - protein, 27 aa, synthetic construct
PAICQRATATLGTVGSNTSGTTEIEAC

SEQ ID NO: 320 - protein, 15 aa, synthetic construct
  MOD_RES 1: note = AcIle
IALILEPICCQERAA

SEQ ID NO: 321 - protein, 30 aa, synthetic construct; identical to SEQ ID NO: 278
  MOD_RES 1: note = AcPro

SEQ ID NO: 322 - protein, 30 aa, synthetic construct; identical to SEQ ID NO: 302
  MOD_RES 1: note = AcPro

SEQ ID NO: 323 - protein, 30 aa, synthetic construct; identical to SEQ ID NO: 303
  MOD_RES 1: note = AcPro

SEQ ID NO: 324 - protein, 30 aa, synthetic construct; identical to SEQ ID NO: 246
  MOD_RES 1: note = AcPro

SEQ ID NO: 325 - protein, 30 aa, synthetic construct; identical to SEQ ID NO: 305
  MOD_RES 1: note = AcPro

SEQ ID NO: 326 - protein, 15 aa, synthetic construct
  MOD_RES 1: note = AcPro
PAICQRAEIEACILL

SEQ ID NO: 327 - protein, 15 aa, synthetic construct
  MOD_RES 1: note = AcPro
PAICQRAEIEACILL

SEQ ID NO: 328 - protein, 15 aa, synthetic construct
  MOD_RES 1: note = AcPro
PAIAQRAEIEAAILL

SEQ ID NO: 329 - protein, 15 aa, synthetic construct
  MOD_RES 1: note = AcIle
IALILEPICCQERAA

SEQ ID NO: 330 - protein, 27 aa, synthetic construct
  MOD_RES 1: note = AcPro
PAICQRATATLGTNTSGTTEIEACILL

SEQ ID NO: 331 - protein, 23 aa, synthetic construct
  MOD_RES 1: note = AcPro
PAICQRATATLSGTTEIEACILL

SEQ ID NO: 332 - protein, 19 aa, synthetic construct
  MOD_RES 1: note = AcPro
PAICQRATATTEIEACILL

SEQ ID NO: 333 - protein, 15 aa, synthetic construct
  MOD_RES 1: note = AcPro
PAICQRAEIEACILL

SEQ ID NO: 334 - protein, 29 aa, synthetic construct
  MOD_RES 1: note = AcAla
AICQRATATLGTVGSNTSGTTEIEACILL

SEQ ID NO: 335 - protein, 28 aa, synthetic construct
  MOD_RES 1: note = AcIle
ICQRATATLGTVGSNTSGTTEIEACILL

SEQ ID NO: 336 - protein, 27 aa, synthetic construct
  MOD_RES 1: note = AcCys
CQRATATLGTVGSNTSGTTEIEACILL

SEQ ID NO: 337 - protein, 29 aa, synthetic construct
  MOD_RES 1: note = AcPro
PAICQRATATLGTVGSNTSGTTEIEACIL

SEQ ID NO: 338 - protein, 28 aa, synthetic construct
  MOD_RES 1: note = AcPro
PAICQRATATLGTVGSNTSGTTEIEACI

SEQ ID NO: 339 - protein, 27 aa, synthetic construct
  MOD_RES 1: note = AcPro
PAICQRATATLGTVGSNTSGTTEIEAC

SEQ ID NO: 340 - protein, 15 aa, synthetic construct
IVVTAPADLWDWIRA

SEQ ID NO: 341 - protein, 15 aa, synthetic construct
ITVTMPADLWDWIRA

SEQ ID NO: 342 - protein, 15 aa, synthetic construct
IVVTIPADLWDWIRA

SEQ ID NO: 343 - protein, 15 aa, synthetic construct
IVVTLPADLWDWIRA

SEQ ID NO: 344 - protein, 15 aa, synthetic construct
IVVTVPADLWDWIRA

SEQ ID NO: 345 - protein, 15 aa, synthetic construct
IVVTMPADLWDWIMA

SEQ ID NO: 346 - protein, 15 aa, synthetic construct
IVVTMPADLWDWINA

SEQ ID NO: 347 - protein, 15 aa, synthetic construct
IVVTMPADLWDWIQA

SEQ ID NO: 348 - protein, 15 aa, synthetic construct
IHVTIPADLWDWINK

SEQ ID NO: 349 - protein, 14 aa, synthetic construct
IHVTIPADLWDWIN

SEQ ID NO: 350 - protein, 36 aa, synthetic construct
  SITE 4: note = cyclohexyl-L-alanine
  SITE 14: note = 4-fluoro-L-phenylalanine
  SITE 29: note = gamma-carboxyglutamic acid
QSDXDCIHRLLEAXLDPNLTEEQRWEKIXKINDECE

SEQ ID NO: 351 - protein, 37 aa, synthetic construct
  SITE 4: note = cyclohexyl-L-alanine
  SITE 14: note = 4-fluoro-L-phenylalanine
  SITE 29: note = gamma-carboxyglutamic acid
  SITE 31: note = PEG30L
QSDXDCIHRLLEAXLDPNLTEEQRWERIXKXINDECE

SEQ ID NO: 352 - protein, 37 aa, synthetic construct; identical to SEQ ID NO: 351
  SITE 4: note = cyclohexyl-L-alanine
  SITE 14: note = 4-fluoro-L-phenylalanine
  SITE 29: note = gamma-carboxyglutamic acid
  SITE 31: note = PEG20Br

SEQ ID NO: 353 - protein, 37 aa, synthetic construct; identical to SEQ ID NO: 351
  SITE 4: note = cyclohexyl-L-alanine
  SITE 14: note = 4-fluoro-L-phenylalanine
  SITE 29: note = gamma-carboxyglutamic acid
  SITE 31: note = PEG40Br

SEQ ID NO: 354 - protein, 35 aa, synthetic construct
  SITE 3: note = homoleucine
FDXDCIHRLLEAFLDPNLTEQQRWEKIDKINDECE

SEQ ID NO: 355 - protein, 36 aa, synthetic construct
  SITE 4: note = cyclohexyl-L-alanine
  SITE 14: note = 4-fluoro-L-phenylalanine
  SITE 29: note = gamma-carboxyglutamic acid
QSDXDCIHRLLEAXLDPNLTEEQRWERIXKINDECE

SEQ ID NO: 356 - protein, 38 aa, synthetic construct
  SITE 6: note = cyclohexyl-L-alanine
  SITE 19: note = N-epsilon-acetyl-L-Lys
SWQSDXDCIHRLLEAFLDXNLTEEQRWERIDKINDECE

SEQ ID NO: 357 - protein, 38 aa, synthetic construct
  SITE 6: note = cyclohexyl-L-alanine
  MOD_RES 19: note = 40 kDa branched PEG-Lys
SWQSDXDCIHRLLEAFLDKNLTEEQRWERIDKINDECE

SEQ ID NO: 358 - protein, 29 aa, synthetic construct
  VAR_SEQ 3: note = note = selected from A, F, H, K, Q, R, S, W and Y
  VAR_SEQ 4: note = note = selected from A, D, E, F, H, I, K, L, M, N, Q, R, S, T, V and Y
  VAR_SEQ 7: note = note = selected from F, H, I, K, L, M, N, R, S, T, V, W and Y
  VAR_SEQ 11: note = note = selected from A, I, K, L, M, N, R, S, T and V
  VAR_SEQ 16: note = note = selected from N and T
  VAR_SEQ 17: note = note = selected from A, I, T and V
  VAR_SEQ 18: note = note = selected from D, E, G, H, K, N, Q, R, S and T
  VAR_SEQ 20: note = note = selected from I, L, M, R, T and V
  VAR_SEQ 21: note = note = selected from A, S, T and V
  VAR_SEQ 25: note = note = selected from I, M, Q, S, T, V and W
  VAR_SEQ 26: note = note = selected from K and S
  VAR_SEQ 28: note = note = selected from F, L, M and Y
  VAR_SEQ 29: note = note = selected from D and R
EEXXAWXEIHXLPNLXXXQXXAFIXXLXX

SEQ ID NO: 359 - protein, 29 aa, synthetic construct
  VAR_SEQ 3: note = note = selected from A, F, H, K, Q, R, S, W and Y
  VAR_SEQ 4: note = note = selected from A, D, E, F, H, I, K, L, M, N, Q, R, S, T, V and Y
  VAR_SEQ 7: note = note = selected from F, H, I, K, L, M, N, R, S, T, V, W and Y
  VAR_SEQ 11: note = note = selected from A, I, K, L, M, N, R, S, T and V
  VAR_SEQ 16: note = note = selected from N and T
  VAR_SEQ 17: note = note = selected from A, I, T and V
  VAR_SEQ 18: note = note = selected from D, E, G, H, K, N, Q, R, S and T
  VAR_SEQ 20: note = note = selected from I, L, M, R, T and V
  VAR_SEQ 21: note = note = selected from A, S, T and V
  VAR_SEQ 25: note = note = selected from I, M, Q, S, T, V and W
  VAR_SEQ 26: note = note = selected from K and S
  VAR_SEQ 28: note = note = selected from F, L, M and Y
  VAR_SEQ 29: note = note = selected from D and R
EEXXAWXEIHXLPNLXXXQXXAFIXXLXX

SEQ ID NO: 360 - protein, 49 aa, synthetic construct
YYALSDAKEEEPRYKALRGENQDLREKERKYQDKIKKLEEKEKNLEKKS

SEQ ID NO: 361 - protein, 17 aa, synthetic construct
DEARRCYNDPKCSDFVQ

SEQ ID NO: 362 - DNA, 15 bp, synthetic construct
  source 1..15: mol_type = other DNA
ggttggtgtggttgg

SEQ ID NO: 363 - protein, 13 aa, synthetic construct
TVFTSWEEYLDWV

SEQ ID NO: 364 - protein, 13 aa, synthetic construct
  MOD_RES 6: note = 4-Biphenylyl-L-alanine
  MOD_RES 8: note = 4-Biphenylyl-L-alanine
  MOD_RES 7: note = N-Methyl-Alanine
  MOD_RES 9: note = N-Methyl-Alanine
  MOD_RES 1: note = N-terminal Acetyl
  CROSSLNK 1..13: note = Sulfide bond formed between N-terminal Acetyl and Cysteine via their side chain or terminus
FVPTTXAXAEAPC

SEQ ID NO: 365 - protein, 13 aa, synthetic construct
  MOD_RES 7: note = N-Methyl-Phenylalanine
  MOD_RES 8: note = 4-Biphenylyl-L-alanine
  MOD_RES 9: note = N-Methyl-Glutamate
  CROSSLNK 1..13: note = Sulfide bond formed between N-terminal Acetyl and Cysteine via their side chain or terminus
  MOD_RES 1: note = N-terminal Acetyl
FVDTTSFXENSPC

SEQ ID NO: 366 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVSTTXAXADRPC

SEQ ID NO: 367 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Biphenylyl-L-Alanine
  MOD_RES 9: note = N-Methyl-Alanine
FVDTTSFXANSPC

SEQ ID NO: 368 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FVDSTYAXANHPC

SEQ ID NO: 369 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Biphenylyl-L-Alanine
  MOD_RES 9: note = N-Methyl-Alanine
FVDTTSFXAESPC

SEQ ID NO: 370 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Fluoromethyl-Phenylalanine
  MOD_RES 9: note = N-Methyl-Alanine
FVDTTSFFAESPC

SEQ ID NO: 371 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FVDTTSAXAKSPC

SEQ ID NO: 372 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVSTTXAXADSPC

SEQ ID NO: 373 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Biphenylyl-L-Alanine
  MOD_RES 9: note = N-Methyl-Alanine
FVSTTSFXADRPC

SEQ ID NO: 374 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVDTTXAXAESPC

SEQ ID NO: 375 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Trifluoromethyl-Phenylalanine
  MOD_RES 7: note = N-Methyl-Alanine
  MOD_RES 8: note = 4-Trifluoromethyl-Phenylalanine
  MOD_RES 9: note = N-Methyl-Alanine
FVSTTFAFAERPC

SEQ ID NO: 376 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Biphenylyl-L-Alanine
  MOD_RES 9: note = N-Methyl-Alanine
FVSTTSFXAESPC

SEQ ID NO: 377 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Phenylalanine
  MOD_RES 8: note = 4-Biphenylyl-L-Alanine
  MOD_RES 9: note = N-Methyl-Alanine
FVSTTXFXAESPC

SEQ ID NO: 378 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 3,4-Dichloro-Phenylalanine
  MOD_RES 9: note = N-Methyl-Alanine
FVSTTXAFADRPC

SEQ ID NO: 379 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 3-Fluoro-Phenylalanine
  MOD_RES 9: note = N-Methyl-Alanine
FVSTTXAFADRPC

SEQ ID NO: 380 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Biphenylyl-L-Alanine
  MOD_RES 9: note = N-Methyl-Alanine
FVDTTAFXAEAPC

SEQ ID NO: 381 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FVDTTFAXAESPC

SEQ ID NO: 382 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Biphenylyl-L-Alanine
  MOD_RES 9: note = N-Methyl-Alanine
FVDTTAFXAQAPC

SEQ ID NO: 383 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVPTTXAXADRPC

SEQ ID NO: 384 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVSTTXAXAPSPC

SEQ ID NO: 385 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Phenylalanine
  MOD_RES 8: note = 4-Biphenylyl-L-Alanine
  MOD_RES 9: note = N-Methyl-Alanine
FVPTTAFXAEAPC

SEQ ID NO: 386 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FVATTFAXAKAPC

SEQ ID NO: 387 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FVNTTFAXAKAPC

SEQ ID NO: 388 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FVSTTFAXAEAPC

SEQ ID NO: 389 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVSTTXAXAESPC

SEQ ID NO: 390 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 3,4-Dichloro-Phenylalanine
  MOD_RES 9: note = N-Methyl-Alanine
FVDTTXAFAESPC

SEQ ID NO: 391 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FENTTFAXAASPC

SEQ ID NO: 392 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Chloro-Phenylalanine
  MOD_RES 9: note = N-Methyl-Alanine
FVPTTFAFAPAPC

SEQ ID NO: 393 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Chloro-Phenylalanine
  MOD_RES 9: note = N-Methyl-Alanine
FVPTTFAFADAPC

SEQ ID NO: 394 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 3: note = Homoserine
  MOD_RES 7: note = N-Methyl-Alanine
  MOD_RES 8: note = 4-Chloro-Phenylalanine
  MOD_RES 9: note = N-Methyl-Alanine
FVXTTFAFADAPC

SEQ ID NO: 395 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Chloro-Phenylalanine
  MOD_RES 9: note = N-Methyl-Alanine
FVATTFAFANAPC

SEQ ID NO: 396 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Chloro-Phenylalanine
  MOD_RES 9: note = N-Methyl-Alanine
FVPTTVAFAEAPC

SEQ ID NO: 397 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
  MOD_RES 10: note = N-Methyl-Glutamate
FVNTTFAXAEAPC

SEQ ID NO: 398 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 7: note = N-Methyl-Alanine
  MOD_RES 8: note = 4-Biphenylyl-L-Alanine
  MOD_RES 9: note = N-Methyl-Alanine
FVPTTFAXAEAPC

SEQ ID NO: 399 - protein, 13 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVPTTXAXAAAPC

SEQ ID NO: 400 - protein, 15 aa, synthetic construct
  MOD_RES 1: note = N-terminal Acetyl
  CROSSLNK 1..13: note = Sulfide bond formed between N-terminal Acetyl and Cysteine via their side chain or terminus
  MOD_RES 6: note = 4-Biphenylyl-L-Alanine
  MOD_RES 7: note = N-Methyl-Alanine
  MOD_RES 8: note = 4-Biphenylyl-L-Alanine
  MOD_RES 9: note = N-Methyl-Alanine
FVPTTXAXAEAPCGK

SEQ ID NO: 401 - protein, 15 aa, synthetic construct
  CROSSLNK 1..14: note = Sulfide bond formed between N-terminal Acetyl and Cysteine via their side chain or terminus
  MOD_RES 1: note = N-terminal Acetyl
  MOD_RES 3: note = N-Methyl-Phenylalanine
  MOD_RES 8: note = N-Methyl-Glycine
  MOD_RES 11: note = 1,2,3,4-tetrahydroisoquinoline-3-carboxylic acid
  MOD_RES 13: note = N-Methyl-Phenylalanine
FRFSANYGRDXEFCG

SEQ ID NO: 402 - protein, 14 aa, synthetic construct
  CROSSLNK 1..13: note = Sulfide bond formed between N-terminal Acetyl and Cysteine via their side chain or terminus
  MOD_RES 1: note = N-terminal Acetyl
  MOD_RES 3: note = N-Methyl-Alanine
  MOD_RES 8: note = N-Methyl-Alanine
FAASIHWARPDRCG

SEQ ID NO: 403 - protein, 19 aa, synthetic construct
VEPNCDIHVMWEWECFERL

SEQ ID NO: 404 - protein, 4 aa, synthetic construct
KRFK

SEQ ID NO: 405 - protein, 7 aa, synthetic construct
STPTRYS

SEQ ID NO: 406 - protein, 9 aa, synthetic construct
  VARIANT 2: note = Threonine or Serine
CXWLHWWAC

SEQ ID NO: 407 - skipped

SEQ ID NO: 408 - protein, 25 aa, synthetic construct
  SITE 1..25: note = D-amino acids
DDDEKQLKERWYKRWLEYLDEFKKN

SEQ ID NO: 409 - protein, 25 aa, synthetic construct
  SITE 1..25: note = D-amino acids
TEEEKQLKEWWYKHWQEYLEEFKKN

SEQ ID NO: 410 - protein, 20 aa, synthetic construct
GACPPCLWQVLCGGSGSGSG

SEQ ID NO: 411 - protein, 12 aa, synthetic construct
HIHDDLLRYYGW

SEQ ID NO: 412 - protein, 14 aa, synthetic construct
  MOD_RES 13: note = Side Chain Modification
  MOD_RES 14: note = Side Chain Modification
HIHFFLLRYYGWLL

SEQ ID NO: 413 - protein, 7 aa, synthetic construct
KRWSRYF

SEQ ID NO: 414 - protein, 12 aa, synthetic construct
HIHDDLLRYYGW

SEQ ID NO: 415 - protein, 9 aa, synthetic construct
YCWSQYLCY

SEQ ID NO: 416 - protein, 15 aa, synthetic construct
DFLPHYKNTSLGHRP

SEQ ID NO: 417 - protein, 9 aa, synthetic construct
YCLYQSWCY

SEQ ID NO: 418 - protein, 7 aa, synthetic construct
SVWIWYE

SEQ ID NO: 419 - protein, 10 aa, synthetic construct
DVWIINKKLK

SEQ ID NO: 420 - protein, 19 aa, synthetic construct
MLRTKDLIWTLFFLGTAVS

SEQ ID NO: 421 - protein, 25 aa, synthetic construct
MLRTKDLIWTLFFLGTAVSKKRPKP

SEQ ID NO: 422 - protein, 25 aa, synthetic construct
MLRTKDLIWTLFFLGTAVSKKLVFF

SEQ ID NO: 423 - protein, 17 aa, synthetic construct
DEARRCYNDPKCSDFVQ

(19) United States
(12) Patent Application Publication
(10) Pub. No.: US 2026/0083851 A1
(43) Pub. Date: Mar. 26, 2026

(54) POTENT ASGPR-BINDING HETEROBIFUNCTIONAL COMPOUNDS FOR THE DEGRADATION OF IMMUNOGLOBULINS AND OTHER PROTEINS

(71) Applicant: AVILAR THERAPEUTICS, INC., Waltham, MA (US)

(72) Inventors: Jason Allan Wiles, Madison, CT (US); Srinivasa Karra, Pembroke, MA (US); Hu Liu, Newton, MA (US)

(73) Assignee: AVILAR THERAPEUTICS, INC., Waltham, MA (US)

(21) Appl. No.: 19/179,878

(22) Filed: Apr. 15, 2025

Related U.S. Application Data
(63) Continuation of application No. PCT/US2023/078749, filed on Nov. 3, 2023.
(60) Provisional application No. 63/422,369, filed on Nov. 3, 2022.
(60) Provisional application No. 63/424,442, filed on Nov. 10, 2022.
(60) Provisional application No. 63/449,553, filed on Mar. 2, 2023.

(51) Int. Cl. A61K 47/54; C07K 1/107

(52) U.S. Cl. CPC A61K 47/549; A61K 47/545; C07K 1/1077

(57) ABSTRACT

Extracellular protein degraders and compositions with improved pharmacokinetic properties are provided that have a potent asialoglycoprotein receptor (ASGPR) Binding Ligand bound to an Extracellular Protein Targeting Ligand for the selective degradation of the Target Extracellular Protein, for example immunoglobulin in vivo to treat disorders mediated by the extracellular protein.

### CROSS REFERENCE TO RELATED APPLICATIONS

This application is a continuation of International Patent Application No. PCT/US2023/078749, filed in the U.S. Receiving Office on Nov. 3, 2023, which claims the benefit of U.S. Provisional Application No. 63/449,553, filed Mar. 2, 2023, U.S. Provisional Application No. 63/424,442, filed Nov. 10, 2022, and U.S. Provisional Application No. 63/422,369, Nov. 3, 2022. The entirety of each of these applications is hereby incorporated by reference for all purposes.

### FIELD OF THE INVENTION

This invention provides extracellular protein degraders and compositions that have an asialoglycoprotein receptor (ASGPR) Binding Ligand bound to an Extracellular Protein Targeting Ligand for the selective degradation of the Target Extracellular Protein for example an immunoglobulin or other extracellular protein in vivo to treat disorders mediated by that protein.

### INCORPORATION BY REFERENCE

The contents of the XML file named “19121-027WO1_ST26” which was created on Nov. 2, 2023, and is 559,122 bytes in size, are hereby incorporated by reference in their entirety.

### BACKGROUND OF THE INVENTION

Historically, therapeutic strategies for the inhibition of proteins employed small molecule inhibitors which bound in an enzymatic pocket or at an allosteric position. Those proteins which are not enzymes are difficult to control, and some are considered “not druggable.” However, many non-enzymatic proteins remain valuable targets for drug discovery because of their role in signaling pathways. Immunoglobulins represent an important non-enzymatic drug target because of their role in signaling immune responses throughout the body.

The asialoglycoprotein receptor (ASGPR) is a Ca²⁺-dependent lectin that is primarily expressed in parenchymal hepatocyte cells. The main role of ASGPR is to help regulate serum glycoprotein levels by mediating endocytosis of desialylated glycoproteins. The receptor binds ligands with a terminal galactose or N-acetylgalactosamine. Asialoglycoproteins bind to ASGPRs and are then cleared by receptor-mediated endocytosis. The receptor and the protein are dissociated in the acidic endosomal compartment and the protein is eventually degraded by lysosomes. Publications describing various utilizations of the ASGPR mechanism include: U.S. Pat. Nos. 9,340,553; 9,617,293; 10,039,778; 10,376,531, and 10,813,942 assigned to Pfizer Inc.; Sanhueza et al. (JACS, 2017, 139, 3528); Petrov et al. (Bioorganic and Medicinal Chemistry Letters, 2018, 28, 382); WO 2018/223073 and WO2018/223081 assigned to Pfizer Inc. and Wave Life Sciences Ltd.; WO 2018/223056 assigned to Wave Sciences Ltd.; Schmidt et al. (Nucleic Acids Research, 2017, 45, 2294); Huang et al. (Bioconjugate Chem. 2017, 28, 283); WO 2019/199621, WO 2019/199634; Banik et al. (Nature, 2020, 584, 291); WO 2020/132100 assigned to The Board of Trustees of the Leland Stanford Junior University; WO 2021/072246, WO 2021/072269, WO 2021/142377 assigned to Lycia Therapeutics; WO 2022/192478, WO 2022/178425 assigned to Yale University; WO 2022/084331 assigned to Sanofi; WO2022/192478 assigned to Biohaven Therapeutics; and an article from the Bertozzi group titled “LYTACs that engage the asialoglycoprotein receptor for targeted protein degradation,” (Ahn, et al. Nat. Chem. Biol. (2021)) published in the journal Nature Chemical Biology.

Additional heterobifunctional compounds which utilize ASGPR-mediated endocytosis to degrade extracellular proteins are described in WO2021/155317 filed by Avilar Therapeutics Inc. Avilar has also disclosed compounds in WO2022/035997 which assemble in vivo to form targeted protein degraders.

While some progress has been made in the area of targeted degradation of extracellular proteins, there remains a need for additional therapeutic compounds and methods for their use and manufacture for the degradation of extracellular proteins to treat disorders mediated by those proteins.

### SUMMARY OF THE INVENTION

Novel extracellular protein degraders and pharmaceutically acceptable salts and compositions thereof that degrade a Target Extracellular Protein, for example IgG, IgA, IgE, TNF-alpha, Factor XIa, complement factor D, complement factor B or other proteins as described below as well as starting materials and intermediates for such extracellular protein degraders and their methods of use and manufacture are provided. The extracellular protein degraders of the present invention contain an ASGPR Binding Ligand covalently attached by a Linker to an Extracellular Protein Targeting Ligand. The ASGPR Binding Ligands used in the degraders described herein include derivatives of six-carbon pyranose moieties, specifically galactose and talose. These two sugars, shown below, differ only in the stereochemistry of the C² substituent. The “down” C² configuration corresponds to the stereochemistry of galactose, while the C² substituent in the “up” configuration corresponds to the stereochemistry of talose. It has been discovered that certain substituents at the C² position of these two sugars improves the binding of the ligand to ASGPR.

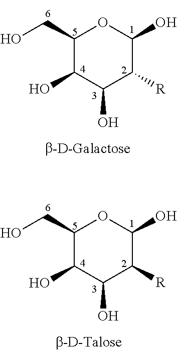

While traditional medicinal chemistry approaches to treat diseases associated with extracellular proteins have failed due to their extracellular circulation, size, and/or lack of active site, the extracellular protein degraders of the present invention can degrade a Target Extracellular Protein by trafficking the protein to the hepatocytes. In some embodiments, these immunoglobulin degraders feature select ASGPR ligands that feature high binding affinity for ASGPR (see as non-limiting examples Table 1 and Table 2). As a result of this high ASGPR binding affinity, the extracellular protein degraders of the present invention can often be administered in lower doses, have fewer side effects, decreased side effects, increased efficacy, faster therapeutic effect, longer metabolic stability, and/or longer therapeutic benefit than previously disclosed immunoglobulin degraders.

In certain aspects an extracellular protein degrading compound of Formula I, Formula II, or Formula III is provided:

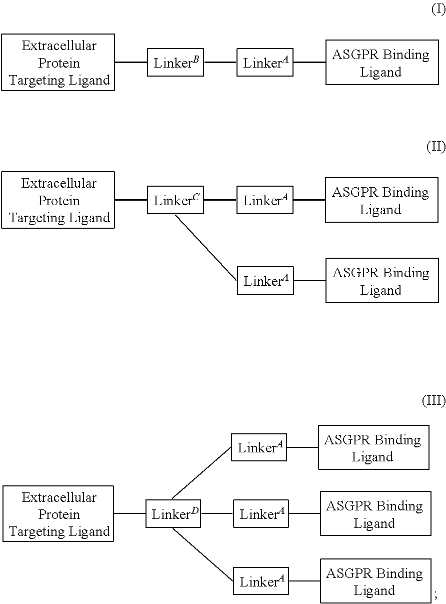

or a pharmaceutically acceptable salt thereof;
wherein the ASGPR Binding Ligand is selected from:

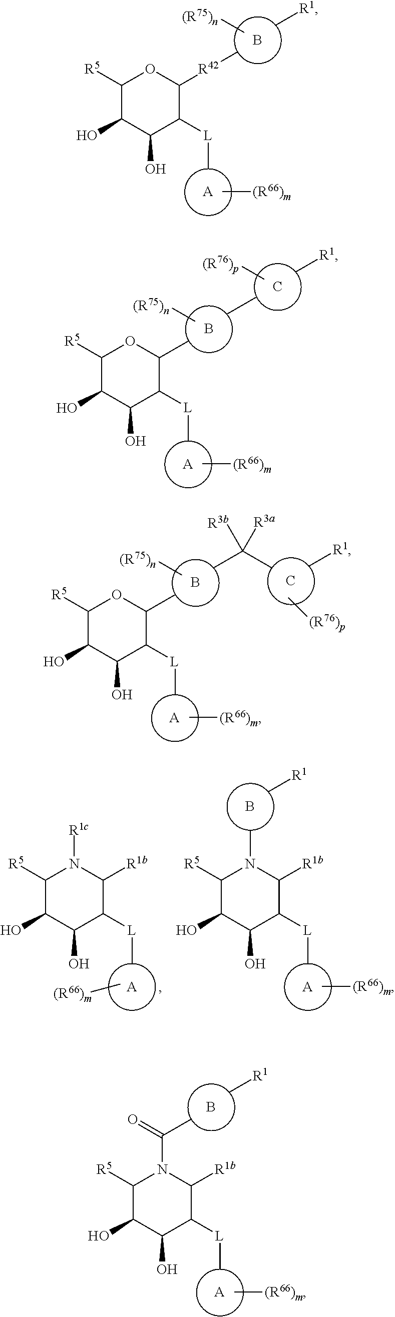

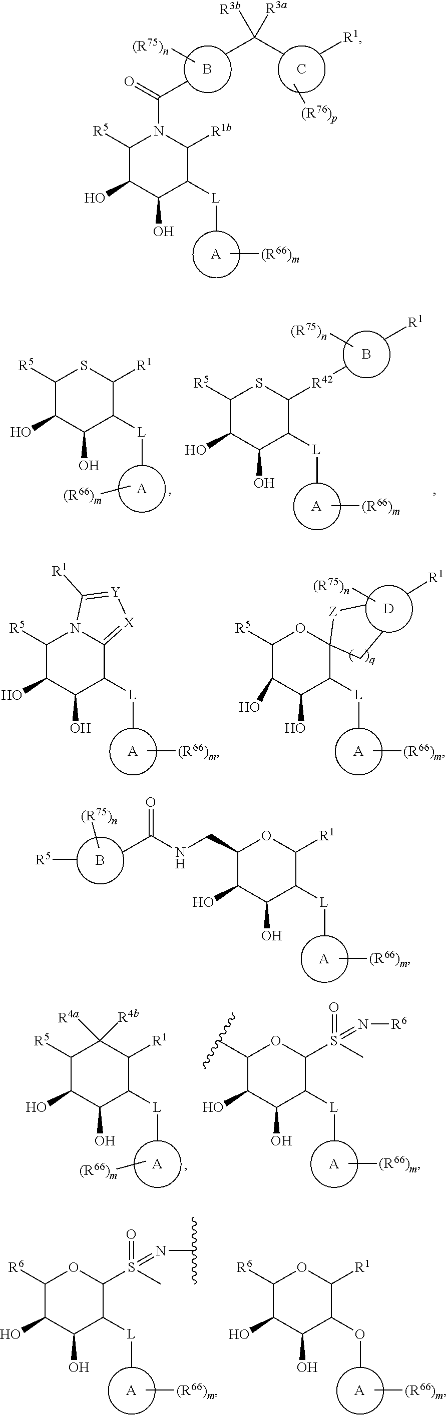

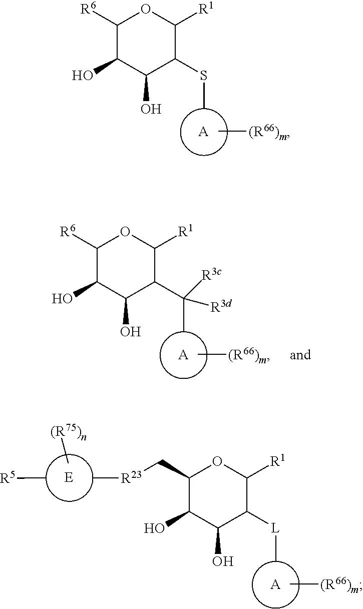

or ASGPR Binding Ligand is selected from:

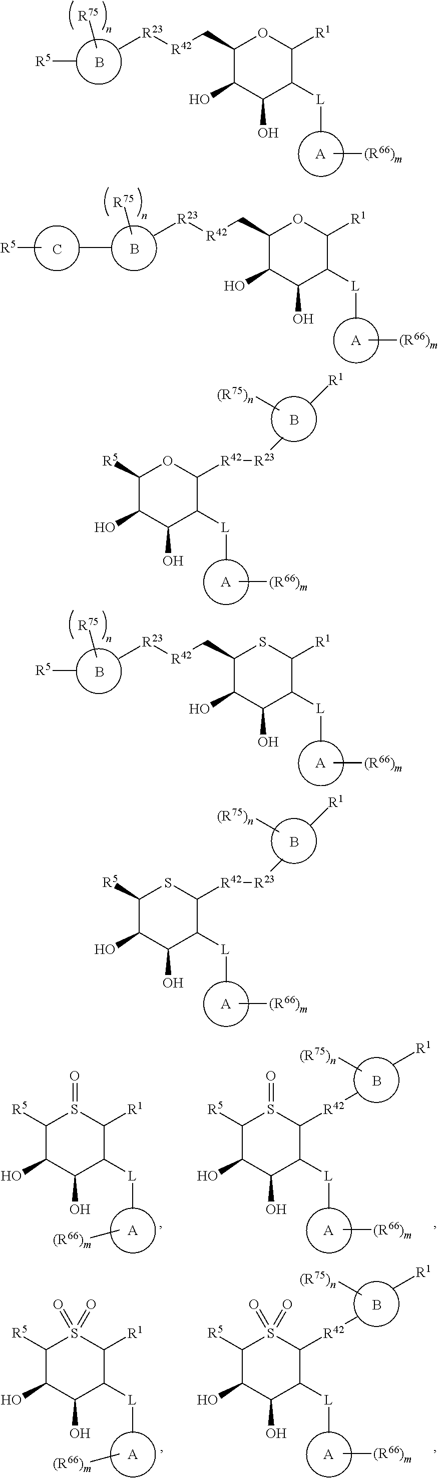

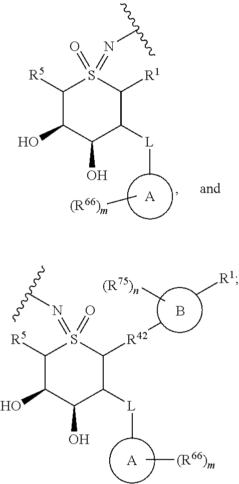

R¹, R^{1b}, and R⁵ are independently selected from hydrogen, C₀-C₆alkyl-cyano, alkyl, alkenyl, alkynyl, haloalkyl, F, Cl, Br, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, and C₀-C₆alkylN₃, each of which except hydrogen, F, Cl, and Br is optionally substituted with 1, 2, 3, or 4 substituents independently selected at each occurrence from R⁹⁹;
R^{1c} is selected from hydrogen, alkyl, alkenyl, alkynyl, haloalkyl, —C(O)R³, —S(O)R³, —C(S)R³, and —S(O)₂R³;
wherein one of R¹, R^{1b}, R^{1c}, and R⁵ is replaced with a bond to Linker^{A};
L is selected from

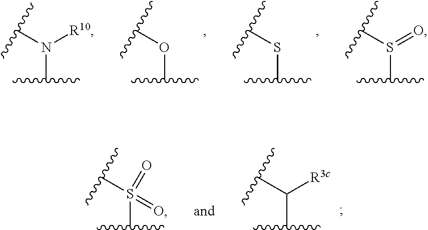

R³, R^{3a}, R^{3b}, R^{3c}, and R^{3d} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R^{4a} is selected from hydrogen, alkyl, haloalkyl, and halogen;
R^{4b} is selected from hydrogen, alkyl, haloalkyl, halogen, C₀-C₆alkyl-OR⁶, C₀-C₆alkyl-SR⁶, and C₀-C₆alkyl-NR⁶R⁷;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR^{B}, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
R⁴² is selected from bond, C₁-C₄ alkyl, C₁-C₄ haloalkyl, C₂-C₄ alkenyl, and C₂-C₄ alkynyl;
R²³ is selected from bond,

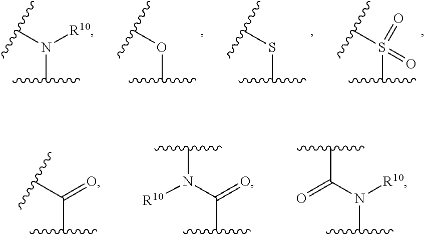

C₁-C₄ alkyl, C₁-C₄ haloalkyl, C₂-C₄ alkenyl, and C₂-C₄ alkynyl;
R⁶⁵, R⁶⁶, and R⁶⁷ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, 3, or 4 substituents independently selected at each occurrence from R¹⁰⁰;
R⁷⁵, R⁷⁶, R⁷⁷, R⁷⁸, and R⁷⁹ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R¹⁰²;
n, m, and p are independently 0, 1, 2, 3, or 4, as allowed by valence;
q is 1, 2, or 3;
\textcircled{A} is heteroaryl;
or \textcircled{A} is phenyl;
\textcircled{B} is aryl, heterocycle, cycloalkyl, or heteroaryl;
\textcircled{C} is aryl, heterocycle, cycloalkyl, bicycle, or heteroaryl;
\textcircled{D} is aryl or heteroaryl;
\textcircled{E} is a bicycle or spirocycle; non-limiting examples of \textcircled{E} include

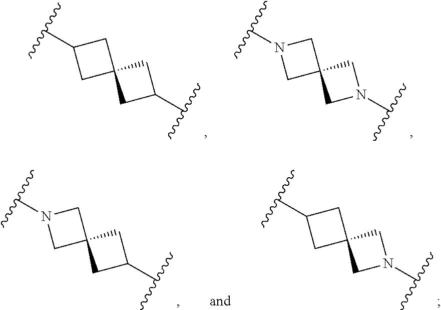

X is CH, CR⁷⁵, or N;
Y is CH, CR⁷⁵, or N;
Z is selected from —O—, —NR⁶—, —S—, —S(O)—, —S(O)₂—, and —CR^{3a}R^{3b}—;
R⁹⁹, R¹⁰⁰, and R¹⁰² are independently selected at each instance from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅ wherein the optional substituent is selected such that a stable compound results;
Linker^{A} is a bond or a moiety that covalently links Linker^{B}, Linker^{C}, or Linker^{D} to the ASGPR Binding Ligand;
Linker^{B} is a bond or a moiety that covalently links Linker^{A} to an Extracellular Protein Targeting Ligand;
Linker^{C} is a chemical group that links each Linker^{A} to the Extracellular Protein Targeting Ligand;
Linker^{D} is a chemical group that links each Linker^{A} to the Extracellular Protein Targeting Ligand; and
Extracellular Protein Targeting Ligand is a Ligand that binds to an extracellular protein.

In certain embodiments, L is selected from

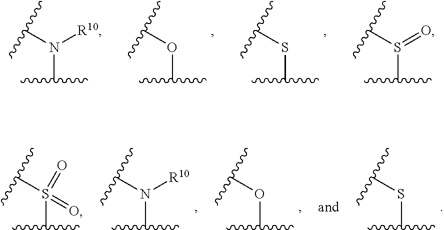

In certain embodiments, L is selected from

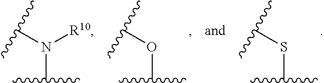

In certain embodiments the ASGPR Binding Ligand is selected from:

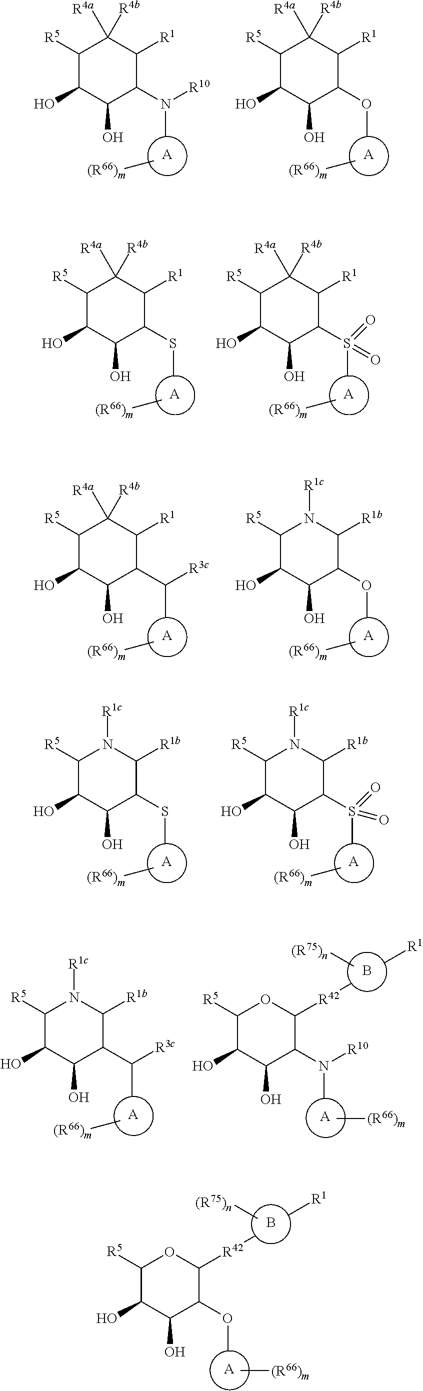

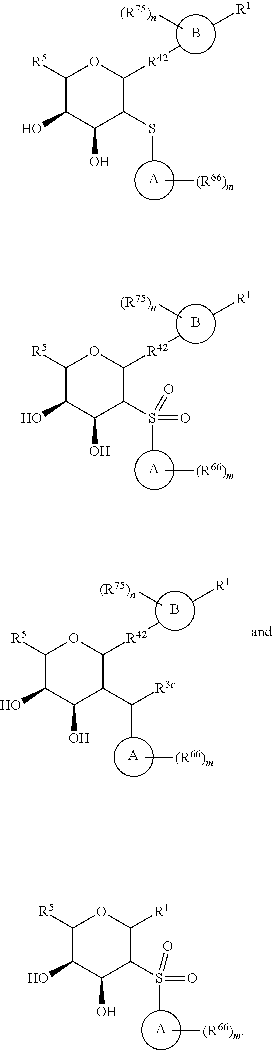

In other embodiments the ASGPR Binding Ligand is selected from:

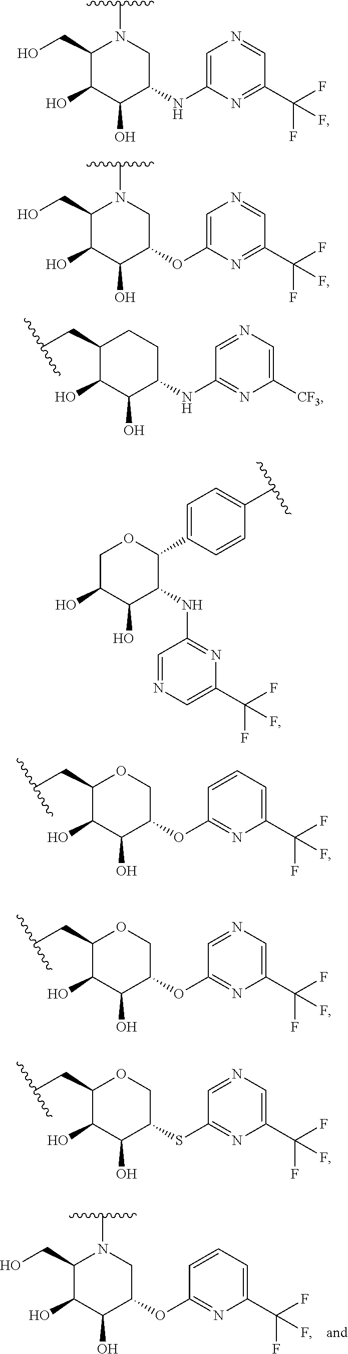

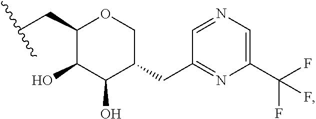 [SMILES: *C[C@H]1OC[C@H](Cc2cncc(C(F)(F)F)n2)[C@@H](O)[C@H]1O]

Additional examples of ASPGR Binding Ligands include:

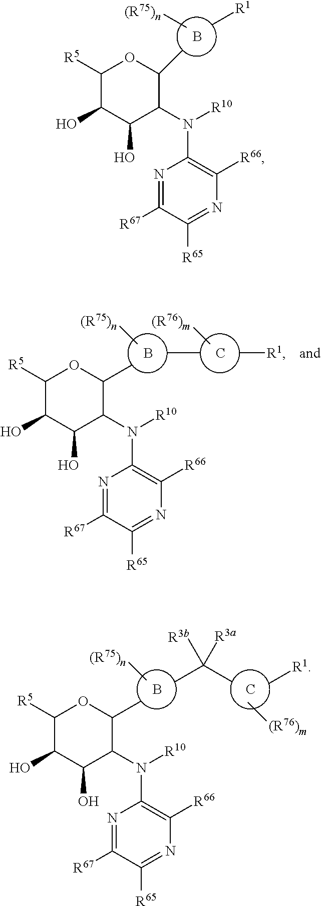

In certain embodiments the ASPGR Binding Ligand is selected from:

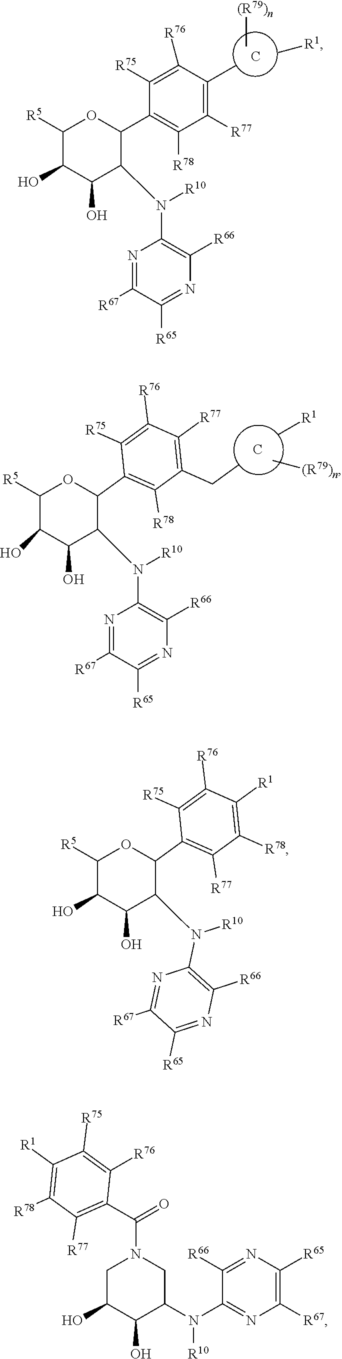

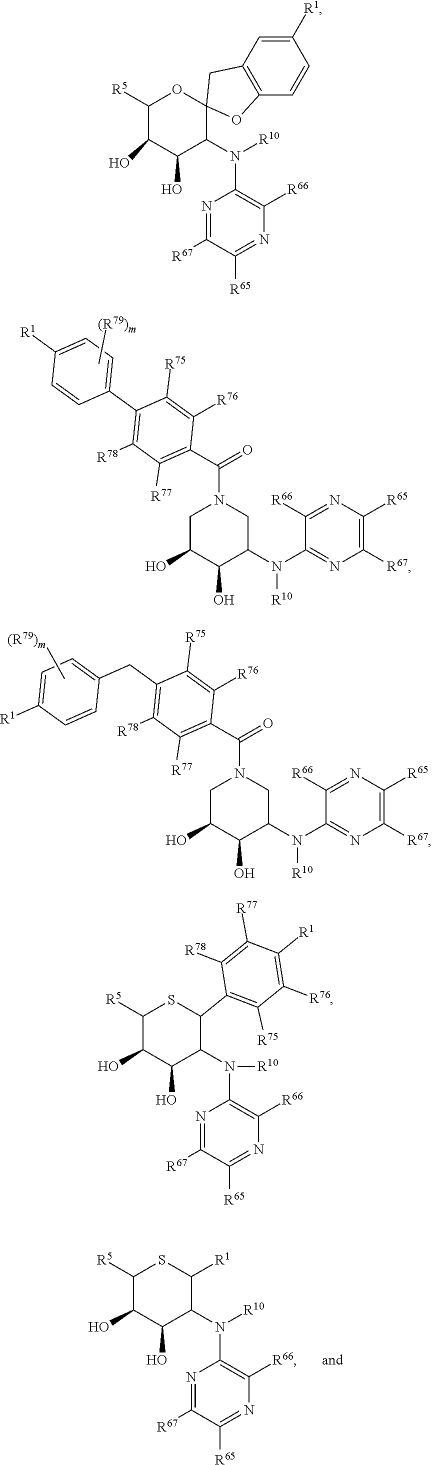

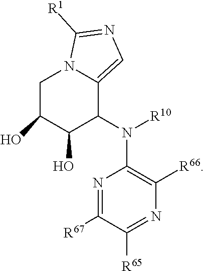

In other embodiments ASGPR Binding Ligand is selected from:

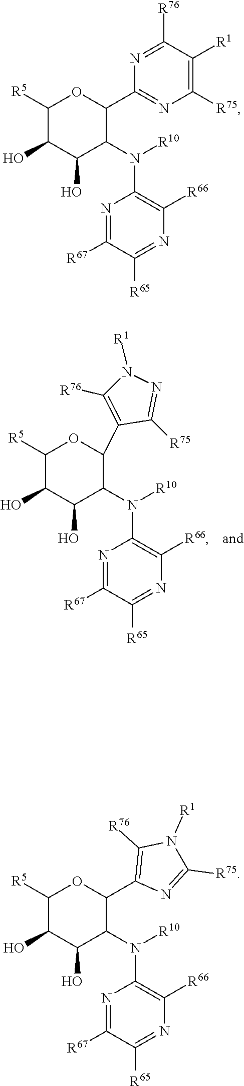

Additional examples of ASGPR Binding Ligands include:

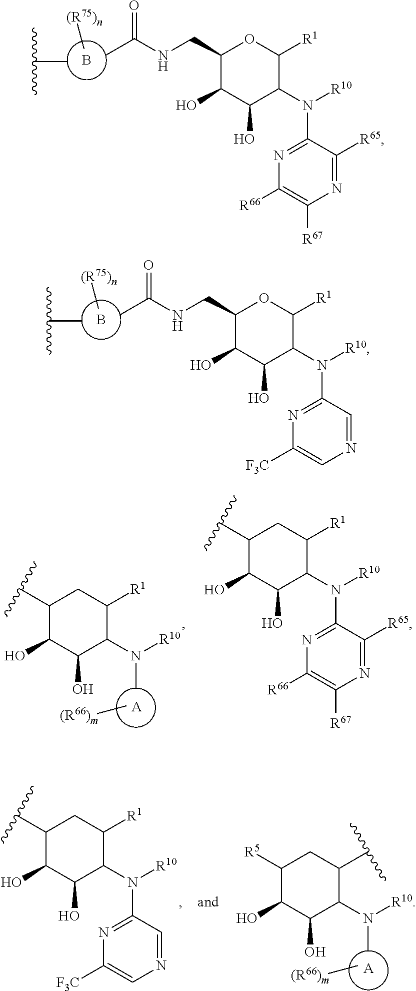

In certain embodiments, the ASGPR Binding Ligand is a compound selected from

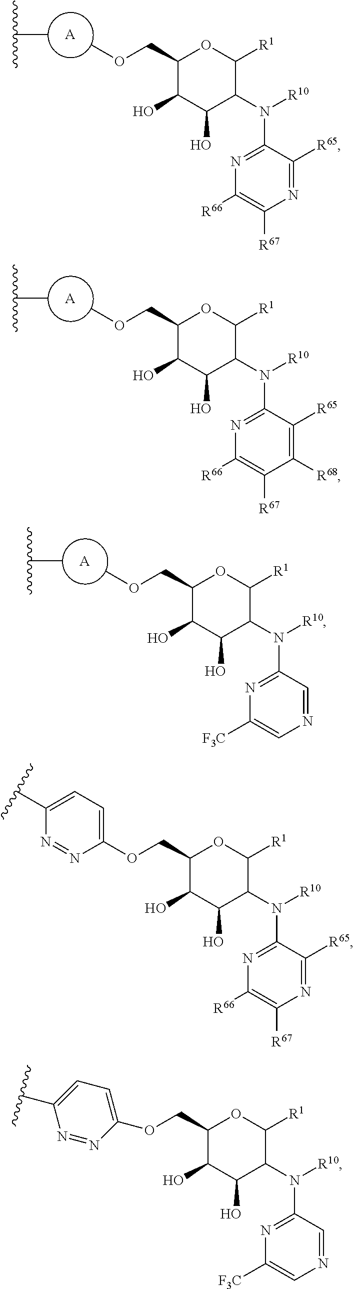

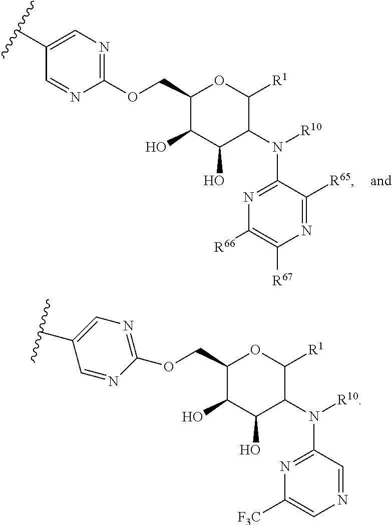

In certain embodiments, the ASGPR Binding Ligand is selected from

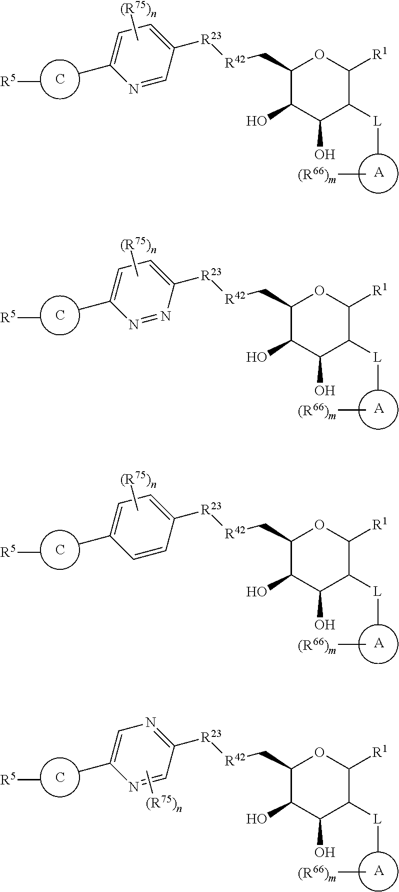

In certain embodiments the ASGPR Binding Ligand is selected from:

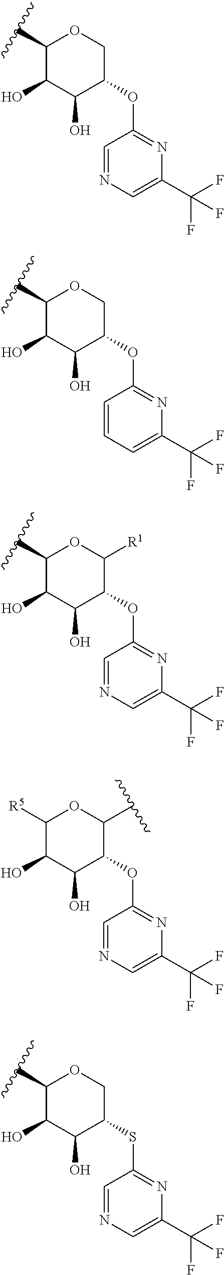

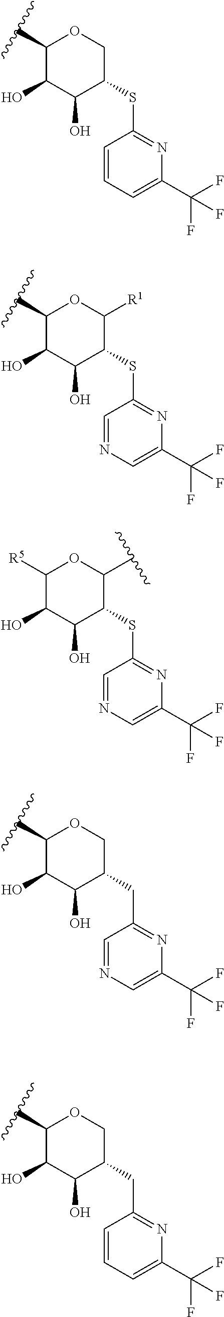

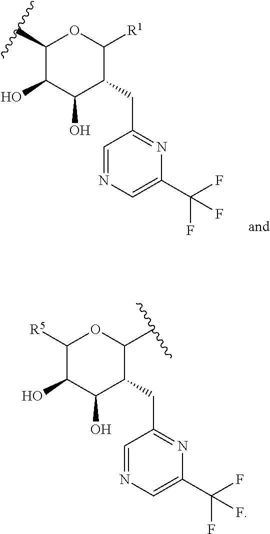

In other embodiments the ASGPR Binding Ligand is selected from:

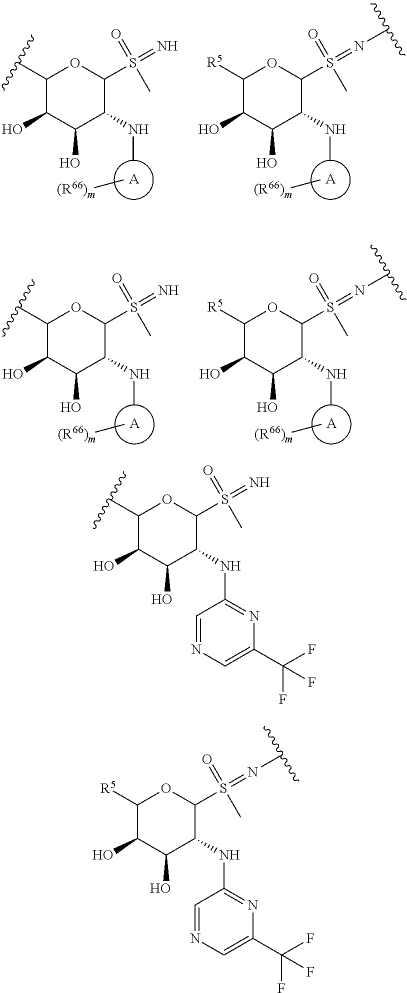

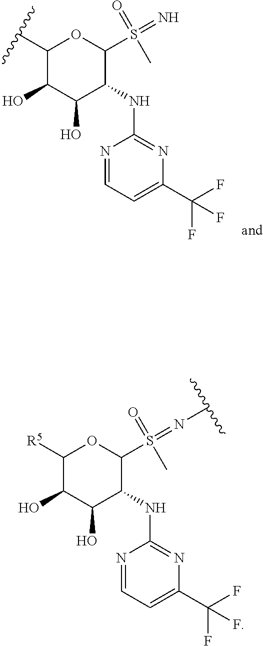

In certain embodiments the Extracellular Protein Degrading Compound of the present invention is selected from

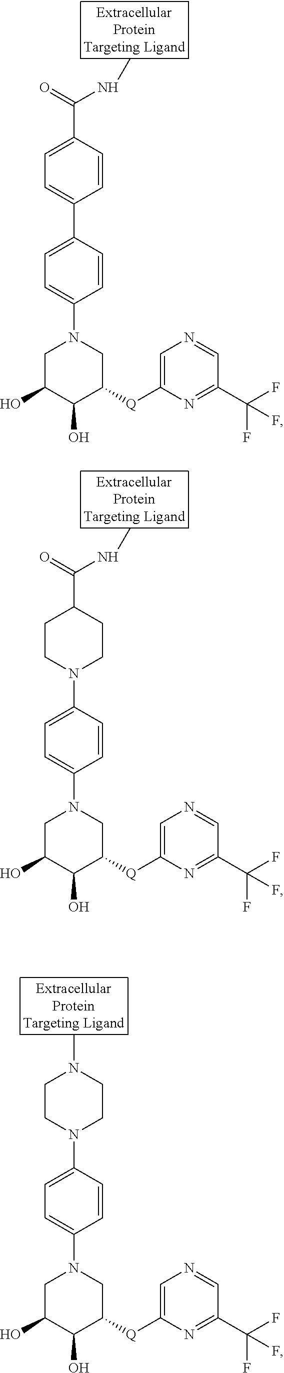

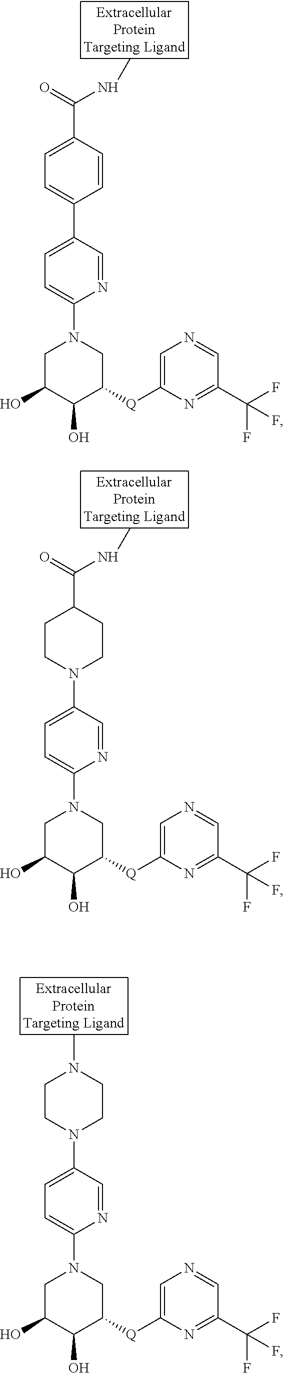

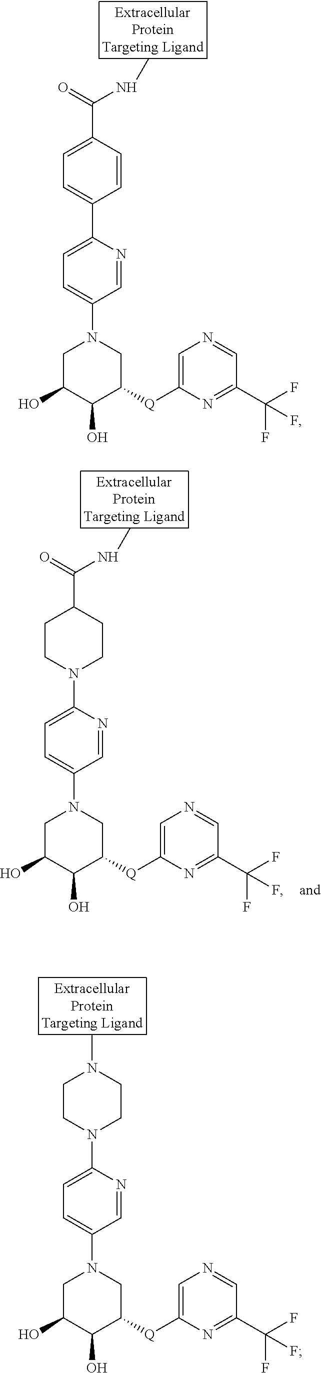

or a pharmaceutically acceptable salt thereof;
wherein Q is NH or O.

In certain embodiments the Extracellular Protein Degrading Compound of the present invention is selected from

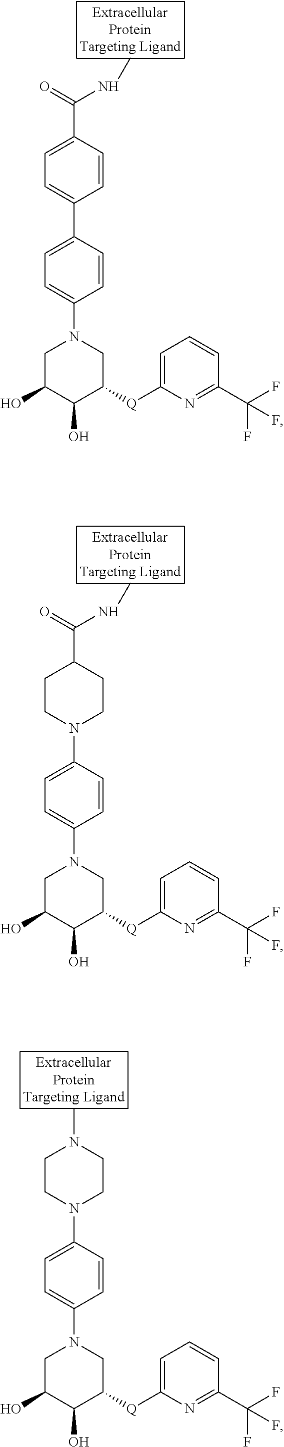

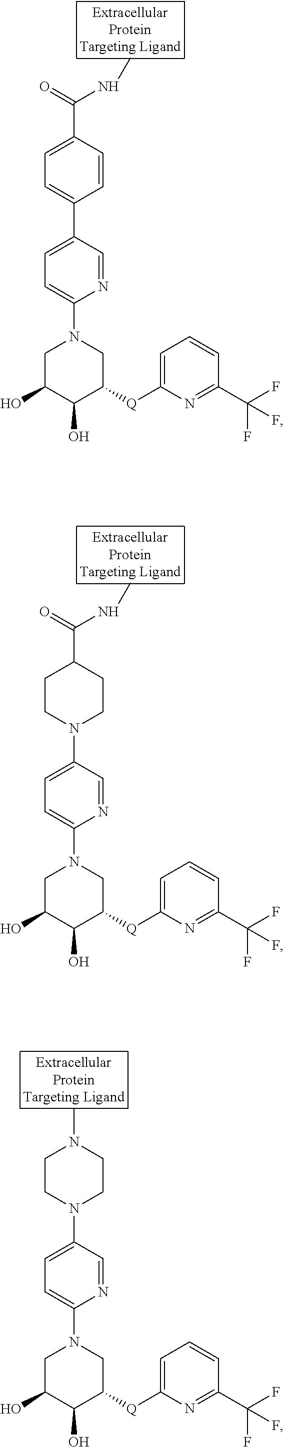

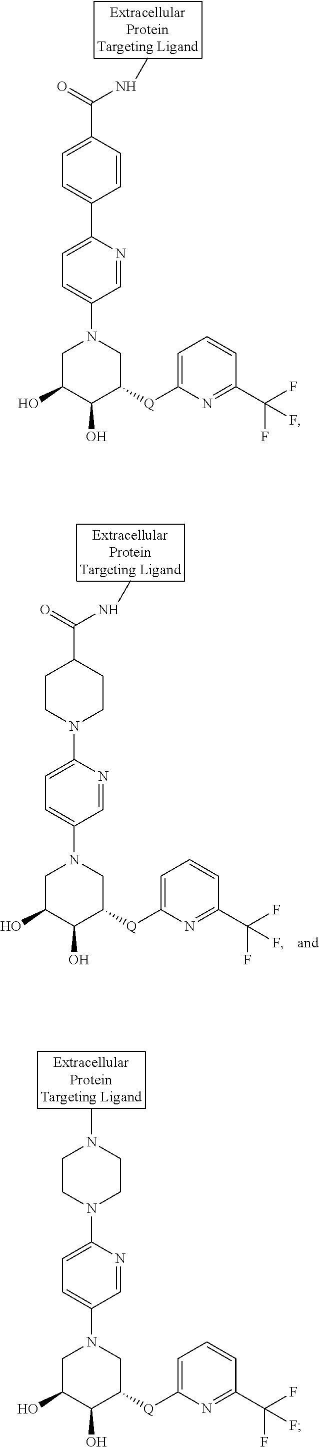

or a pharmaceutically acceptable salt thereof.

In certain embodiments

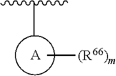

is

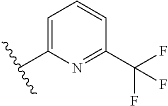 [SMILES: *c1cccc(C(F)(F)F)n1]

In certain embodiments the Extracellular Protein Degrading Compound of the present invention is selected from

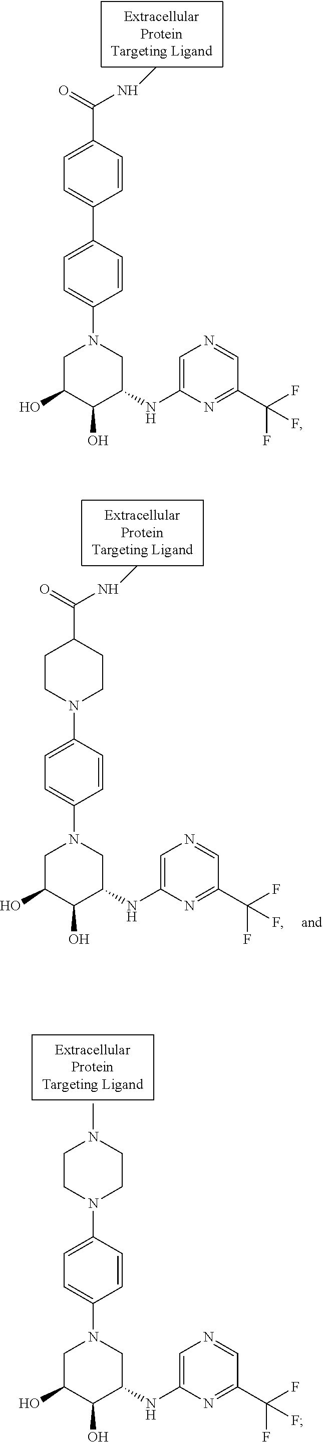 [SMILES: CNC(=O)c1ccc(-c2ccc(N3C[C@H](Nc4cncc(C(F)(F)F)n4)[C@@H](O)[C@@H](O)C3)cc2)cc1 ; CNC(=O)C1CCN(c2ccc(N3C[C@H](Nc4cncc(C(F)(F)F)n4)[C@@H](O)[C@@H](O)C3)cc2)CC1 ; CN1CCN(c2ccc(N3C[C@H](Nc4cncc(C(F)(F)F)n4)[C@@H](O)[C@@H](O)C3)cc2)CC1]

or a pharmaceutically acceptable salt thereof.

In other embodiments the Extracellular Protein Degrading Compound of the present invention is selected from:

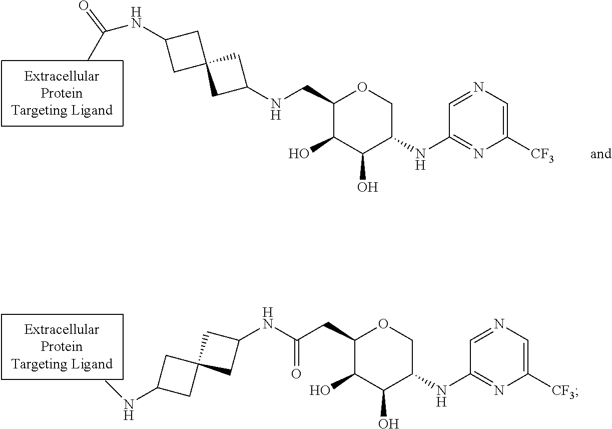

or a pharmaceutically acceptable salt thereof.

In some aspects of the invention, the Extracellular Protein Targeting Ligand targets an immunoglobulin, for example IgG, IgA, IgM, or IgE.

In certain aspects an immunoglobulin degrading compound of Formula I-A, Formula II-A, or Formula III-A is provided:

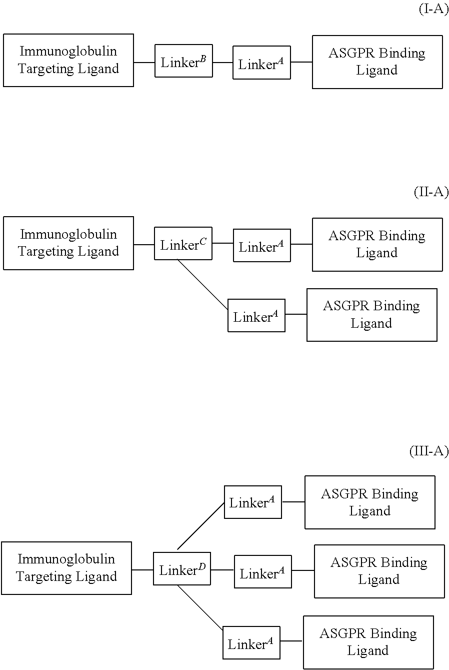

or a pharmaceutically acceptable salt thereof,
wherein:
Immunoglobulin Targeting Ligand is a Ligand that binds to an immunoglobulin, for example IgG, IgA, IgM, or IgE.

The immunoglobulin degrading compounds described herein degrade a target immunoglobulin, for example IgG, IgA, IgM, or IgE, by linking a ligand for the selected immunoglobulin to a potent ASGPR binder through specific linking groups. In certain embodiments of the present invention, the selected immunoglobulin degrader degrades IgG.

In some aspects of the present invention an IgG degrader of the present invention uses a 2:1 ratio of ASGPR Binding Ligand to Extracellular Protein Targeting Ligand. In other aspects of the present invention an IgG degrader of the present invention uses a 1:1 ratio of ASGPR Binding Ligand to Extracellular Protein Targeting Ligand.

In certain embodiments, the immunoglobulin degraders have a peptide-based IgG targeting ligand. In certain embodiments, the peptide-based IgG targeting ligand is the peptide Fc-BP2. For example, an IgG targeting ligand of structure:

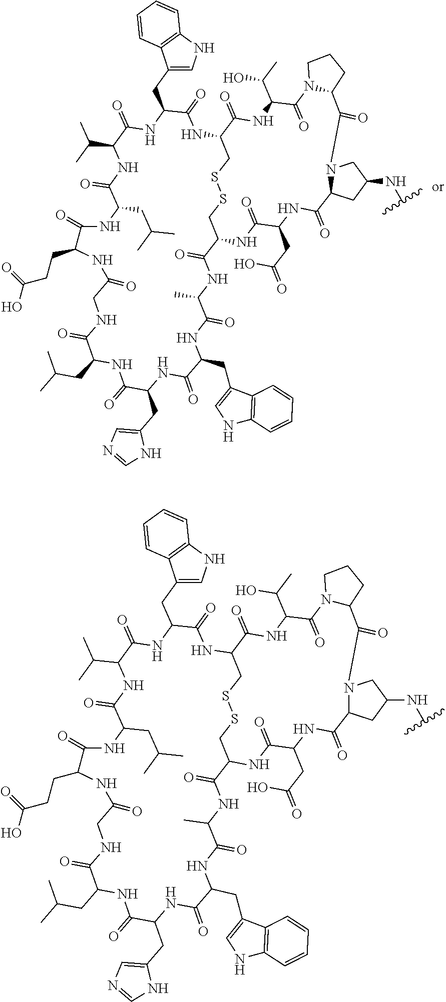

In certain embodiments, the peptide-based IgG targeting ligand is the peptide Fc-III-4C. For example, an IgG targeting ligand of the structure:

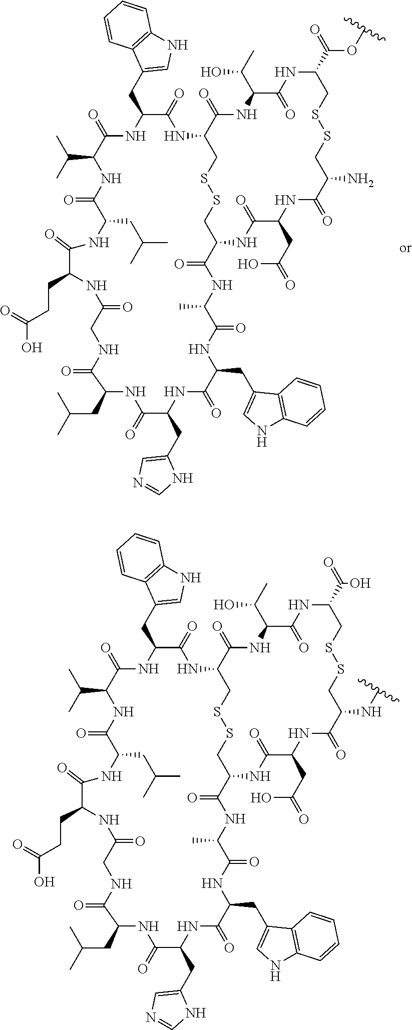

In certain embodiments, the peptide-based IgG targeting ligand is the peptide Fc-III. For example, an IgG targeting ligand of the structure:

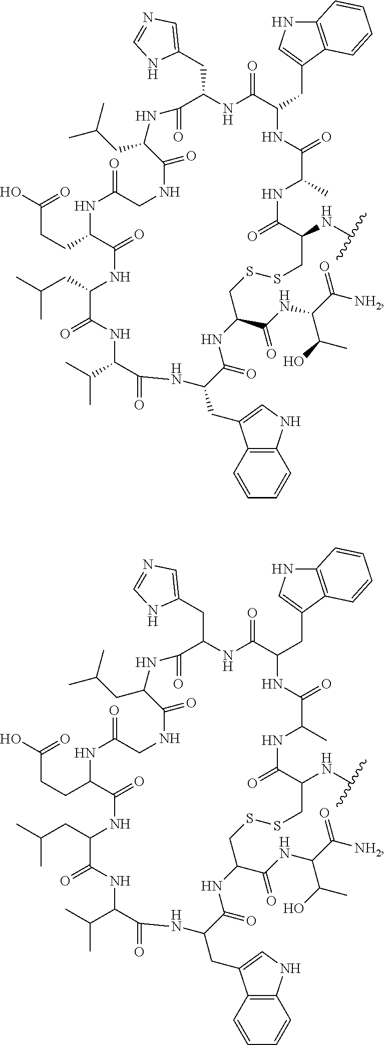

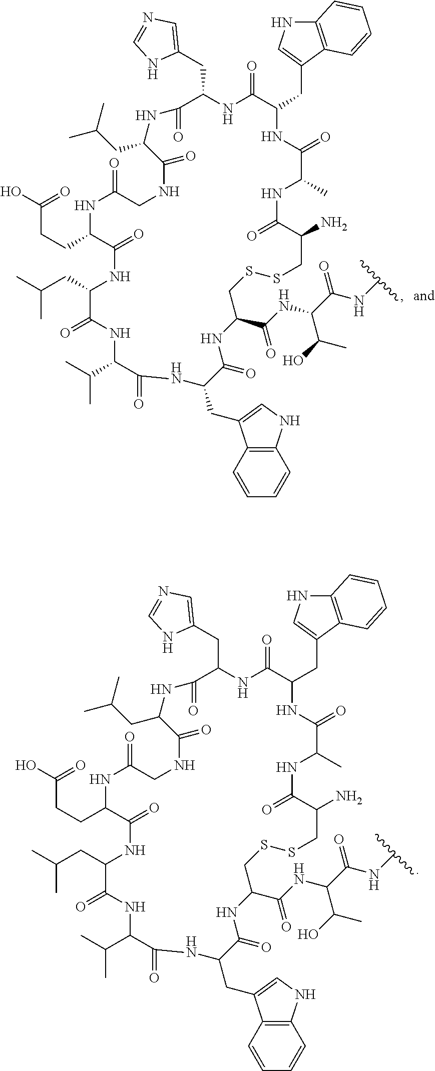

In certain embodiments, these immunoglobulin degraders have a small molecule or nonpeptidic IgG targeting ligand. Non-limiting examples of small molecule IgG targeting ligands include:

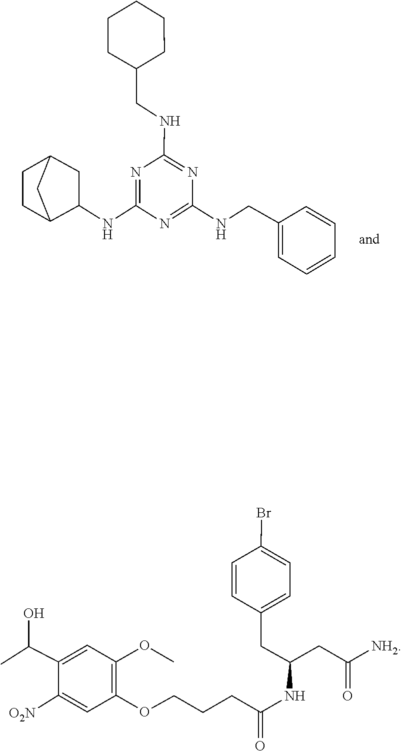 [SMILES: c1ccc(CNc2nc(NCC3CCCCC3)nc(NC3CC4CCC3C4)n2)cc1 ; COc1cc(C(C)O)c([N+](=O)[O-])cc1OCCCC(=O)N[C@H](CC(N)=O)Cc1ccc(Br)cc1]

The selective targeting of IgG can be particularly beneficial when the present invention is used in the treatment of a disease known to be caused primarily by IgG, such as thyroid eye disease, myasthenia gravis, chronic inflammatory demyelinating polyneuropathy, warm autoimmune hemolytic anemia, and type-1 autoimmune pancreatitis.

In certain embodiments a compound of the present invention which degrades IgG is used to treat a disorder selected from Graves' eye disease, Graves' ophthalmopathy, Graves' orbitopathy, thyroid eye disease, neuromyelitis optica spectrum disorder (NMOSD), myelin oligodendrocyte glycoprotein antibody-associated disease (MOGAD).

In certain aspects the treatment of a disorder mediated by IgG is provided comprising administering an effective amount of an IgG degrader or a pharmaceutically acceptable salt thereof to the patient. In certain embodiments the IgG disorder is selected from antiphospholipid Ab syndrome, Behcet syndrome, Hashimoto thyroiditis, MGUS, necrobiotic xanthogranuloma, rheumatoid arthritis, cancer, for example multiple myeloma or peripheral multiple myeloma, paraproteinemia, chronic urticaria, scleroderma, scleromyxedema, thrombocytopenia for example heparin-induced thrombocytopenia, cryoglobulinema, granulomatosis with polyanglititis, for example ANCA associated vasculitis, idiopathic thrombocytopenic purpura, thrombocytopenia, IgG4-RD, paroxysmal nocturnal hemoglobinuria (PNH), warm autoimmune hemolytic anemia, rhabdomyolysis, lupus nephritis, acute disseminated encephalomyelitis, Guillaine-Barre syndrome, chronic inflammatory demyelinating polyneuropathy, Miller Fisher syndrome, neuromyelitis optica spectrum disorder, opsoclonus-myoclonus syndrome, pediatric autoimmune neuropsychiatric disorder associated with streptococcal infection (PANDAS), peripheral neuropathy, transverse myelitis, fibrosis, IPF/fibrosis, and transplantation rejection.

In some aspects of the present invention an IgA degrader uses a 2:1 ratio of ASGPR Binding Ligands to IgA Binding Ligand. In some aspects of the present invention an IgA degrader uses a 1:1 ratio of ASGPR Binding Ligands to IgA Binding Ligand.

In certain embodiments the IgA degrader uses a peptide-based IgA targeting ligand, such as but not limited to OPT-1, OPT-2, or OPT-3. In certain embodiments, the peptide-based IgA targeting ligand is OPT-1. For example, an IgA targeting ligand of structure:

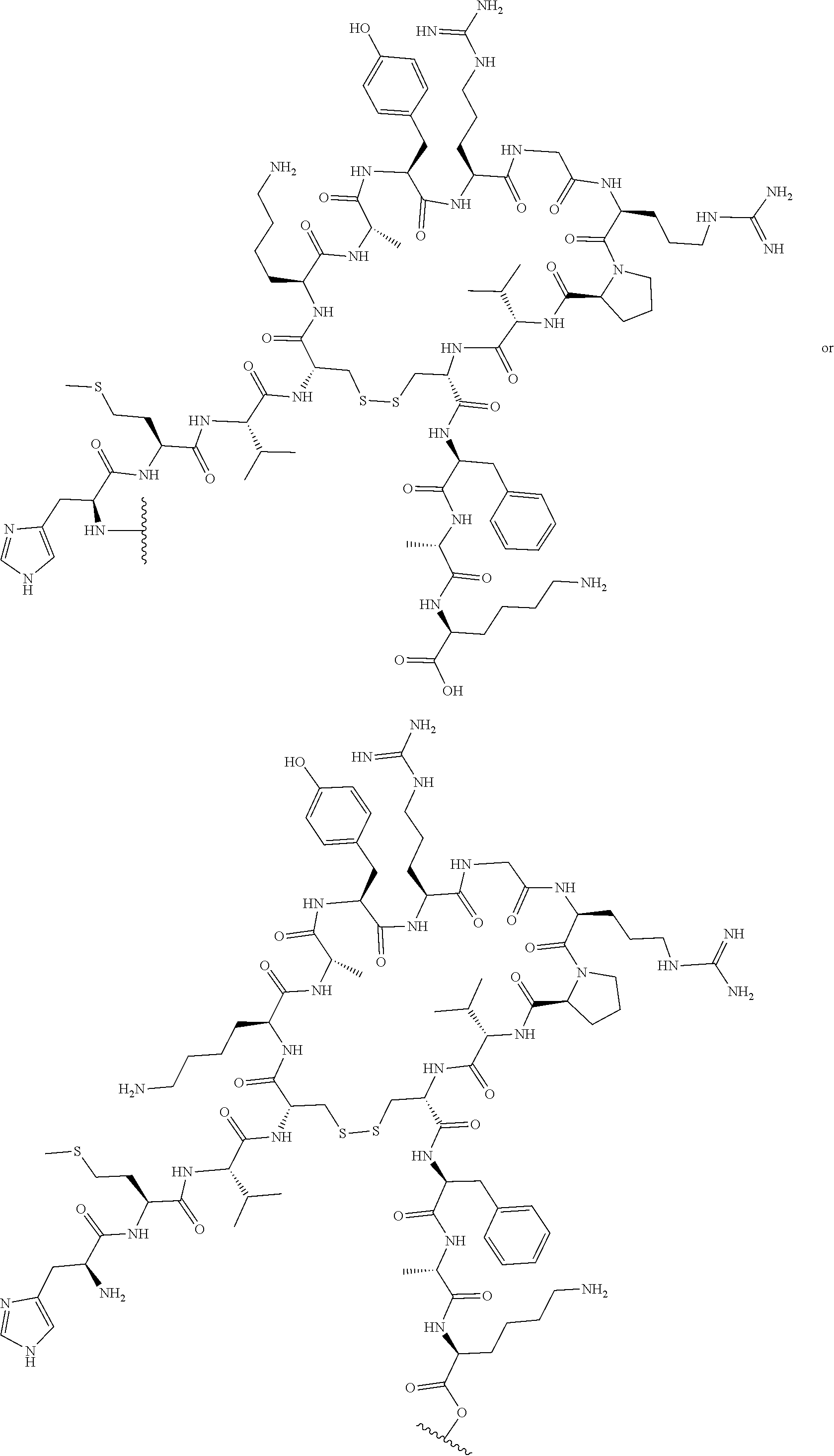

In certain embodiments, the peptide-based IgA targeting ligand is OPT-2. For example, an IgA targeting ligand of structure:

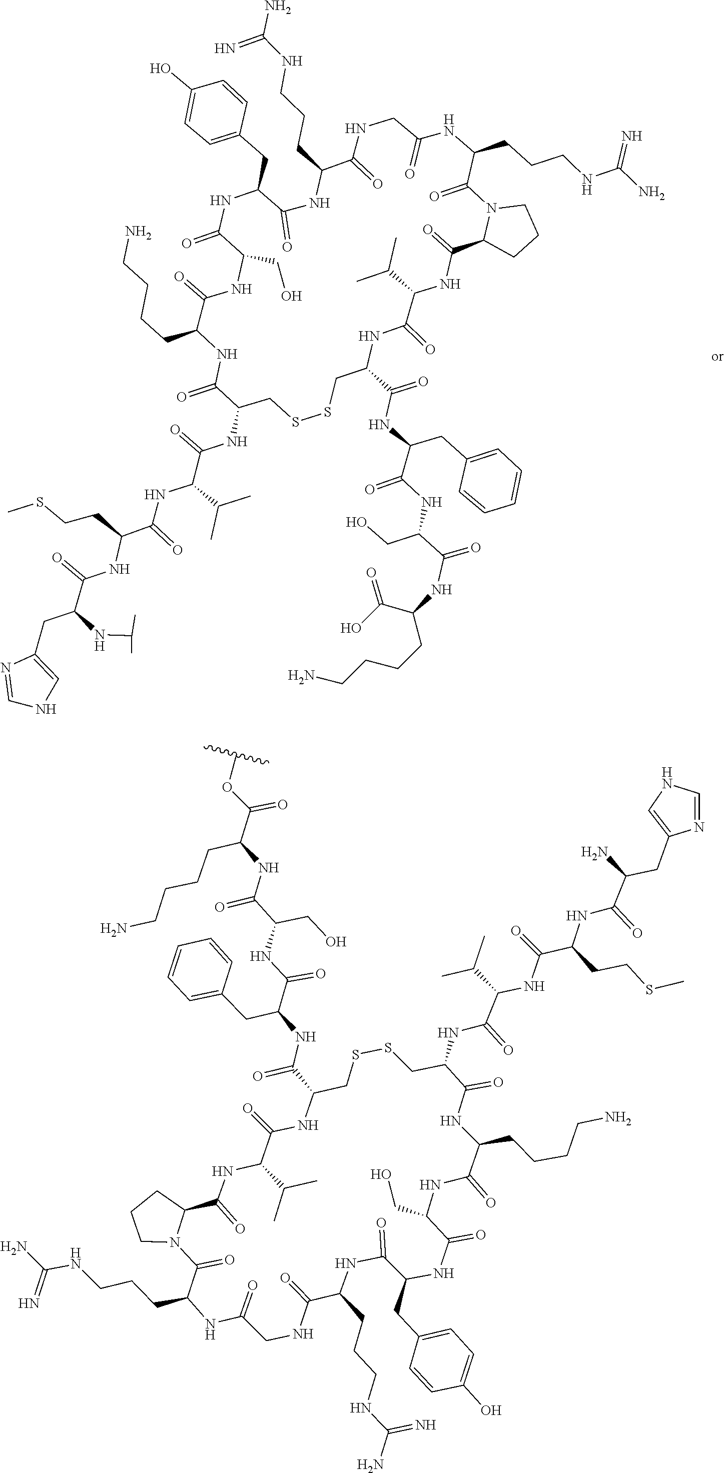

In certain embodiments, the peptide-based IgA targeting ligand is OPT-3. For example, an IgA targeting ligand of structure:

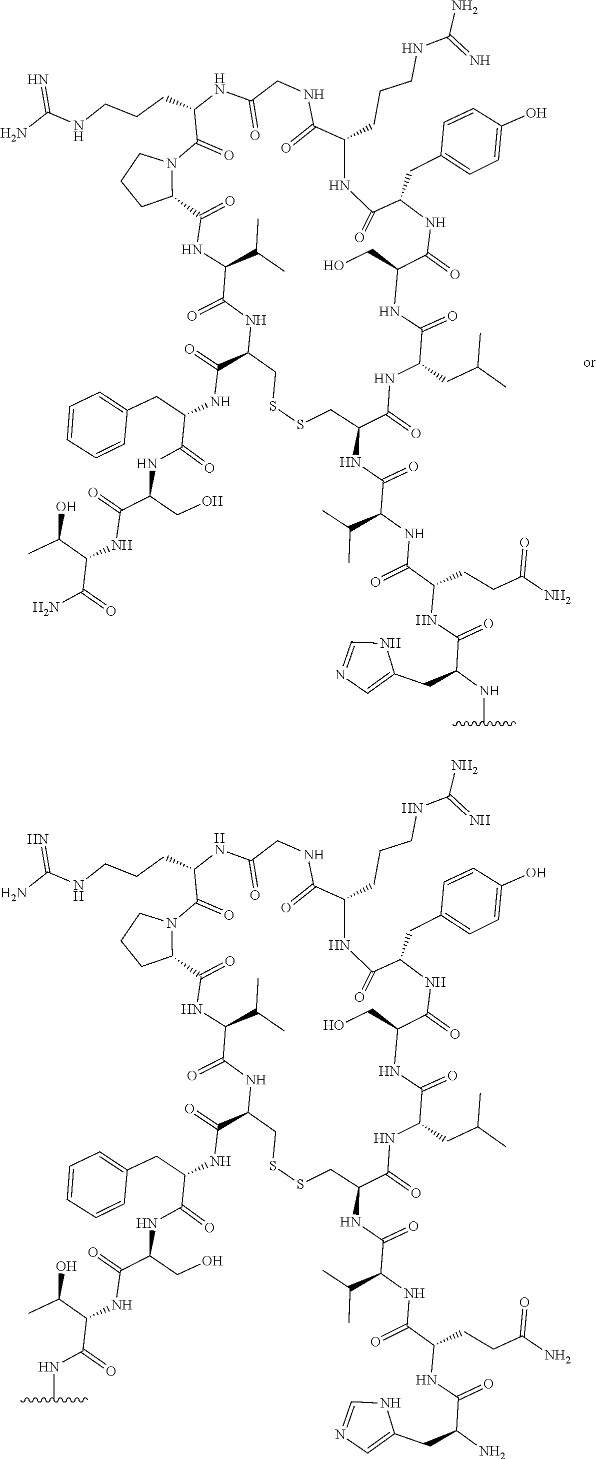

In certain embodiments, the peptide-based IgA targeting ligand is

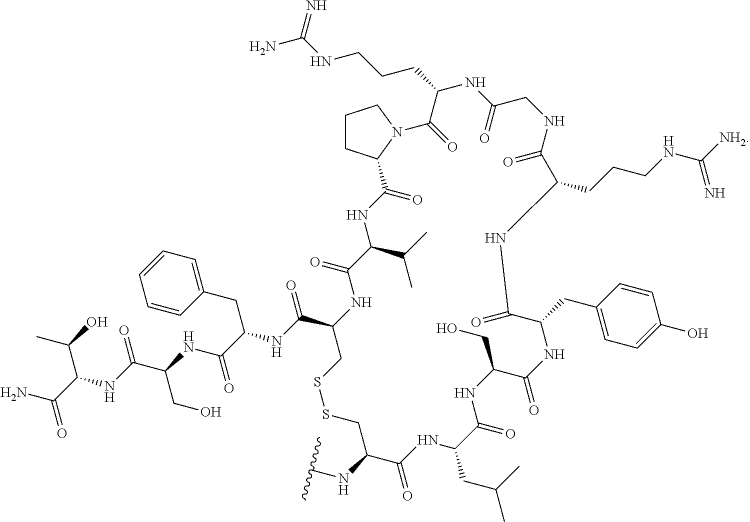 [SMILES: *N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O]

The selective targeting of IgA can be particularly beneficial when the present invention is used in the treatment of a disease known to be caused primarily by IgA, such as Henoch-Schonlein purpura, also known as IgA vasculitis. Additional disorders mediated by IgA include cryoglobulinemia, granulomatosis with polyangiitis, thrombocytopenia, peripheral neuropathy, MGUS, IgA nephropathy, and Henoch Schonlein purpura.

The immunoglobulin degraders described herein can be used to treat a disorder mediated by an immunoglobulin, for example IgG or IgA, including for example an autoimmune disorder, other immune dysfunction, abnormal cellular proliferation such as tumors and cancer, hematology-related disorder, renal disorder, allergic condition, or liver disorder. In certain aspects of the invention, a method for treating a disorder mediated by an immunoglobulin is provided that includes administering to a host in need thereof an effective amount of an immunoglobulin degrader described herein, or its pharmaceutically acceptable salt, prodrug, N-oxide, and/or a pharmaceutically acceptable composition thereof optionally in a pharmaceutically acceptable carrier.

In certain embodiments the extracellular protein degrader of the present invention is provided as an isotopically enriched extracellular protein degrader, for example an immunoglobulin degrader, with at least one desired isotopic substitution of an atom, at an amount above the natural abundance of the isotope. For example, deuterium can replace one or more hydrogens in the extracellular protein degrader and ¹³C can replace one or more carbon atoms. In certain embodiments, the isotopic substitution is in one or more positions of the ASGPR Ligand.

In another embodiment, the isotopic substitution is in one or more positions of the Linker portion of the molecule. In another embodiment, the isotopic substitution is in one or more positions of the Extracellular Protein Targeting Ligand portion of the molecule.

In some embodiments, other extracellular proteins can be degraded as described further below. For example, in non-limiting illustrative embodiments, a selected Extracellular Protein described generally herein can be targeted, for example, where relevant, using a selected Targeting Ligand of FIGS. 1-7 or as otherwise known.

In certain aspects of the invention, an ASGPR ligand selected from the following formulas can be used independently from a heterobifunctional molecule:

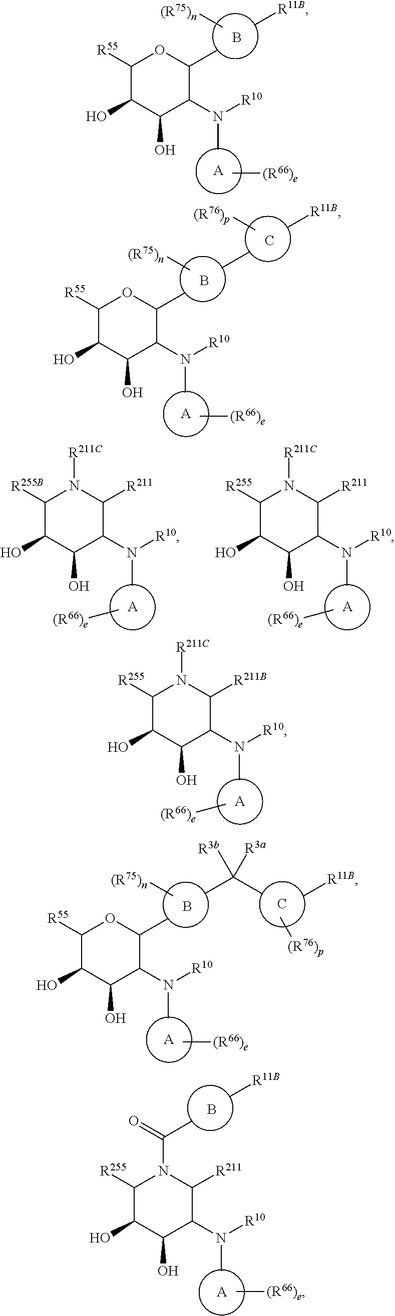

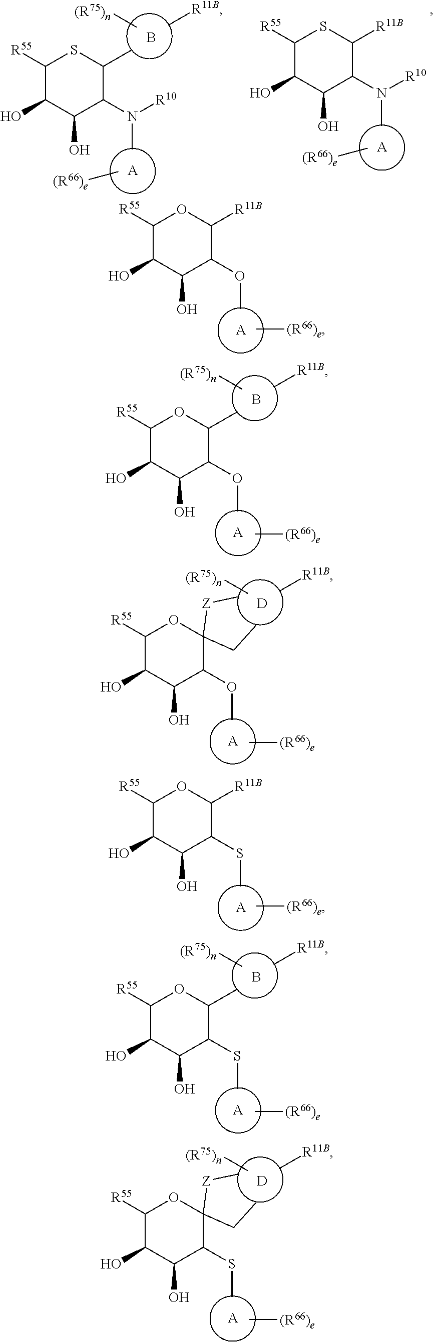

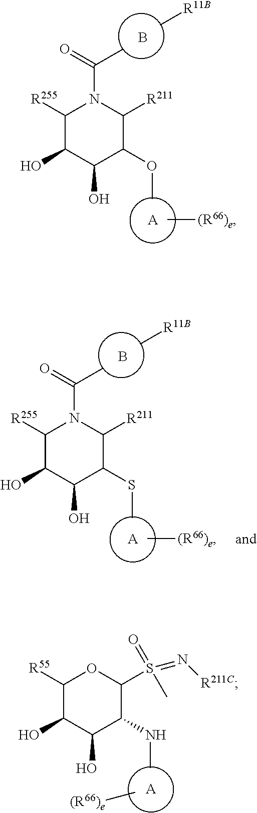

or a salt thereof;
wherein:
R^{11B}, R⁵⁵, and R^{66B} are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkylN₃, and R¹¹⁰ each of which is optionally substituted with 1, 2, 3, or 4 substituents independently selected at each occurrence from R⁹⁹;
e is 0, 1, 2, or 3;
R¹¹⁰ is:

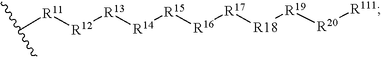

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²1 is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R²⁵⁵, R²¹¹, R^{211C} are independently selected from hydrogen, C₁-C₆alkyl-cyano, alkyl, alkenyl, alkynyl, haloalkyl, aryl, arylalkyl, heteroaryl, heteroarylalkyl, heterocycle, heterocycloalkyl, haloalkoxy, C₁-C₆alkyl-OR⁶, C₁-C₆alkyl-SR⁶, C₁-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₁-C₆alkylN₃, and R¹¹⁰ each of which is optionally substituted with 1, 2, or 3 substituents independently selected at each occurrence from R⁹⁹;
R^{255B} and R^{211B} are independently selected from C₁-C₆alkyl-cyano, alkyl, alkenyl, alkynyl, haloalkyl, aryl, arylalkyl, heteroaryl, heteroarylalkyl, heterocycle, heterocycloalkyl, haloalkoxy, C₁-C₆alkyl-OR⁶, C₁-C₆alkyl-SR⁶, C₁-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₁-C₆alkylN₃, and R¹¹⁰ each of which is optionally substituted with 1, 2, or 3 substituents independently selected at each occurrence from R⁹⁹;
R¹¹¹ is independently selected from the group consisting of hydrogen, alkyl, cycloalkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-O—P(O)₂R³, C₀-C₆alkyl-O—P(O)₃—R³, C₀-C₆alkyl-O—P(O)(OR³)₂, and C₀-C₆alkylN₃, each of which is optionally substituted with 1, 2, 3, or 4 substituents independently selected at each occurrence from R¹⁰³;

In certain embodiments one of R^{11B}, R⁵⁵, and R^{66B} is R¹¹⁰. In other embodiments none of R^{11B}, R⁵⁵, and R^{66B} are R¹¹⁰. In certain embodiments one of R²⁵⁵, R²¹¹, and R^{211C} is R¹¹⁰. In other embodiments none of R²⁵⁵, R²¹¹, and R^{211C} are R¹¹⁰. In certain embodiments one of R^{255B} and R^{211B} is R¹¹⁰. In other embodiments none of R^{255B} and R^{211B} are R¹¹⁰.

In other aspects an ASGPR ligand selected from the following formulas can be used independently from a heterobifunctional molecule:

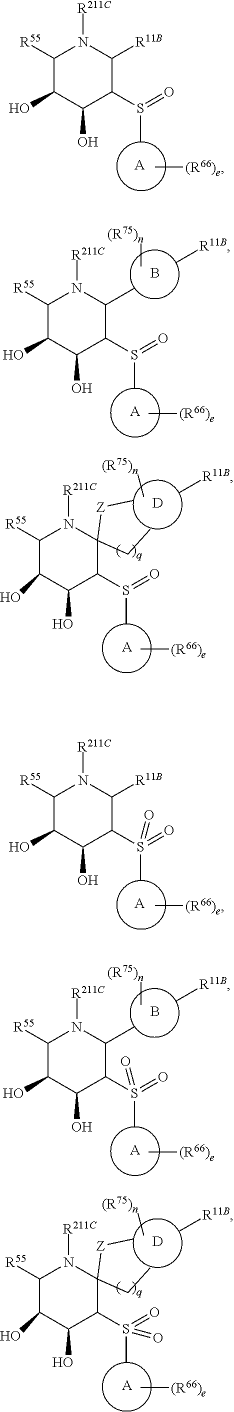

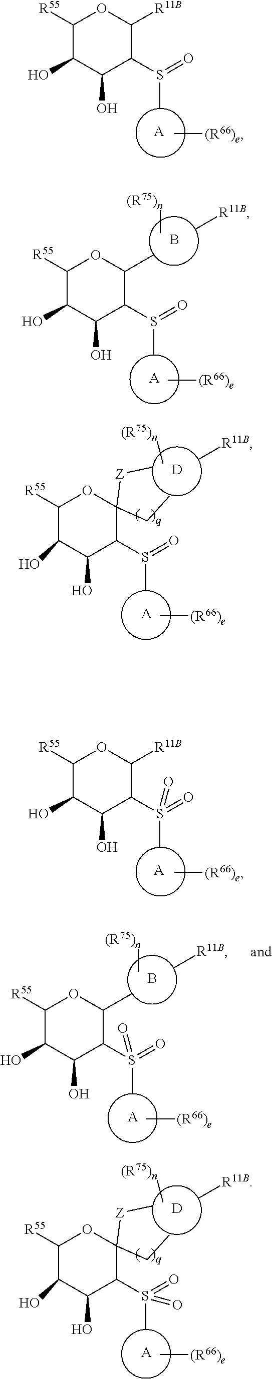

Additional non-limiting examples of ASGPR ligands of the present invention include:

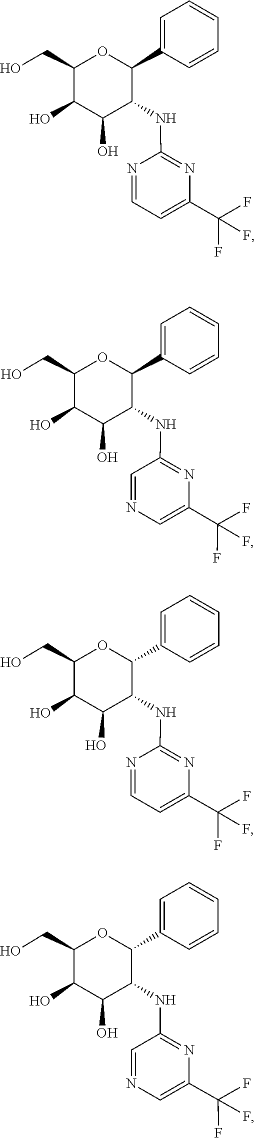

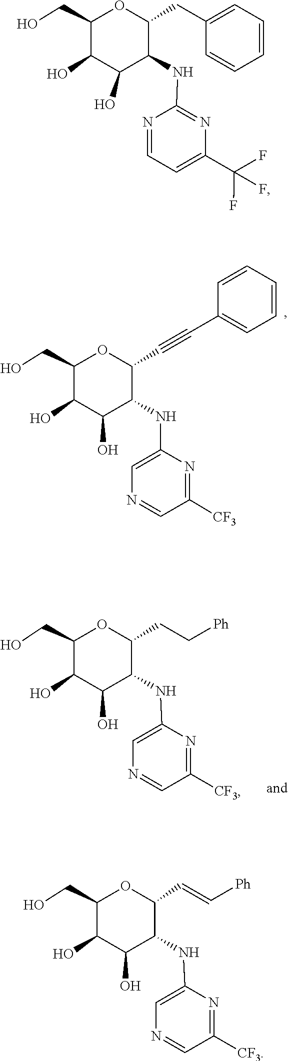 [SMILES: OC[C@H]1O[C@H](Cc2ccccc2)[C@@H](Nc2nccc(C(F)(F)F)n2)[C@@H](O)[C@H]1O ; OC[C@H]1O[C@H](C#Cc2ccccc2)[C@H](Nc2cncc(C(F)(F)F)n2)[C@@H](O)[C@H]1O ; OC[C@H]1O[C@H](CCc2ccccc2)[C@H](Nc2cncc(C(F)(F)F)n2)[C@@H](O)[C@H]1O ; OC[C@H]1O[C@H](/C=C/c2ccccc2)[C@H](Nc2cncc(C(F)(F)F)n2)[C@@H](O)[C@H]1O]

Non-limiting examples of ASGPR ligands of the present invention include:

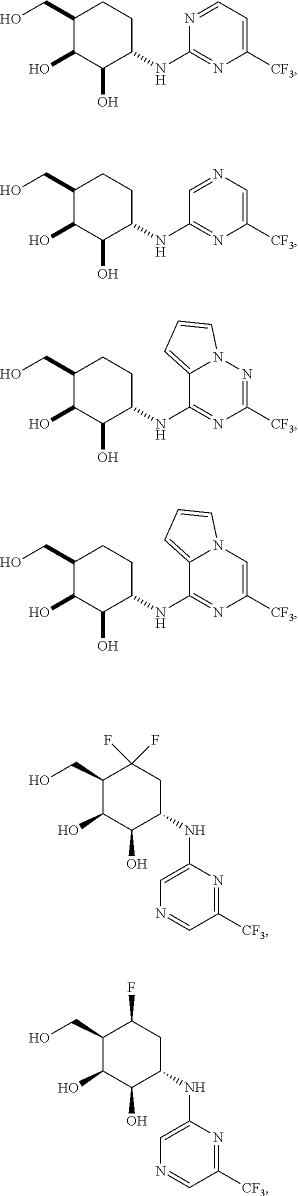 [SMILES: OC[C@H]1CC[C@H](Nc2nccc(C(F)(F)F)n2)[C@@H](O)[C@H]1O ; OC[C@H]1CC[C@H](Nc2cncc(C(F)(F)F)n2)[C@@H](O)[C@H]1O ; OC[C@H]1CC[C@H](Nc2nc(C(F)(F)F)nn3cccc23)[C@@H](O)[C@H]1O ; OC[C@H]1CC[C@H](Nc2nc(C(F)(F)F)cn3cccc23)[C@@H](O)[C@H]1O ; OC[C@@H]1[C@H](O)[C@H](O)[C@@H](Nc2cncc(C(F)(F)F)n2)CC1(F)F ; OC[C@@H]1[C@H](O)[C@H](O)[C@@H](Nc2cncc(C(F)(F)F)n2)C[C@@H]1F]

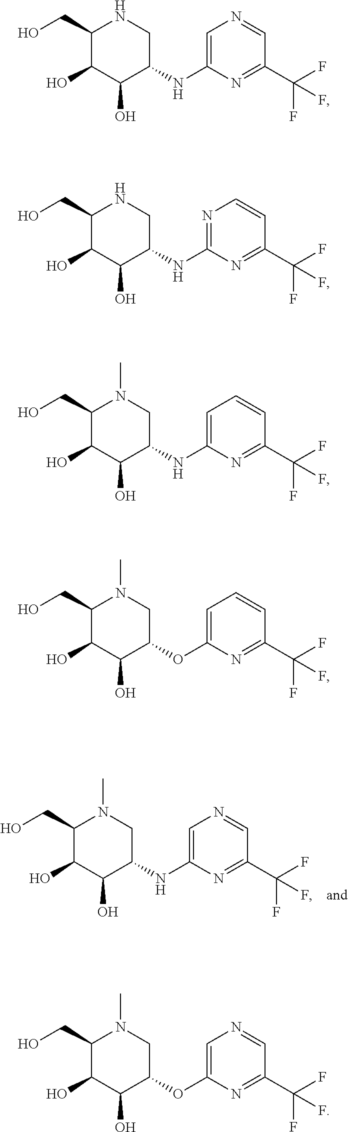 [SMILES: OC[C@H]1NC[C@H](Nc2cncc(C(F)(F)F)n2)[C@@H](O)[C@H]1O ; OC[C@H]1NC[C@H](Nc2nccc(C(F)(F)F)n2)[C@@H](O)[C@H]1O ; CN1C[C@H](Nc2cccc(C(F)(F)F)n2)[C@@H](O)[C@@H](O)[C@H]1CO ; CN1C[C@H](Oc2cccc(C(F)(F)F)n2)[C@@H](O)[C@@H](O)[C@H]1CO ; CN1C[C@H](Nc2cncc(C(F)(F)F)n2)[C@@H](O)[C@@H](O)[C@H]1CO ; CN1C[C@H](Oc2cncc(C(F)(F)F)n2)[C@@H](O)[C@@H](O)[C@H]1CO]

The present invention thus includes at least the following features:
(i) An extracellular protein degrader described herein or a pharmaceutically acceptable salt thereof, prodrug, N-oxide, and/or a pharmaceutical composition thereof as described herein;
(ii) An extracellular protein degrader described herein for use in treating a medical disorder which is associated with the extracellular protein;
(iii) An isotopically enriched derivative of an extracellular protein degrader described herein or pharmaceutically acceptable salt, prodrug, N-oxide, and/or a pharmaceutical composition thereof;
(iv) A process for manufacturing a medicament intended for the therapeutic use for treating or preventing a disorder mediated by an extracellular protein, characterized in that an extracellular protein degrader described herein is used in the manufacture;
(v) An extracellular protein degrader described herein or a salt thereof as described herein in purified or substantially pure form (e.g., at least 90, 95, 96, 97, 98, 99, 99.5, or 99.9%);
(vi) A method for the manufacture of an extracellular protein degrader described herein;
(vii) An immunoglobulin degrader described herein or a pharmaceutically acceptable salt thereof, prodrug, N-oxide, and/or a pharmaceutical composition thereof as described herein;
(viii) An immunoglobulin degrader described herein for use in treating a medical disorder which is associated with an immunoglobulin, such as an autoimmune disorder, other immune dysfunction, hematology-related disorder, renal disorder, allergic condition, or liver disorder;
(ix) An isotopically enriched derivative of an immunoglobulin degrader described herein or pharmaceutically acceptable salt, prodrug, N-oxide, and/or a pharmaceutical composition thereof;
(x) A process for manufacturing a medicament intended for the therapeutic use for treating or preventing a disorder mediated by an immunoglobulin, characterized in that an immunoglobulin degrader described herein is used in the manufacture;
(xi) An immunoglobulin degrader described above or a salt thereof as described herein in purified or substantially pure form (e.g., at least 90, 95, 96, 97, 98, 99, 99.5, or 99.9%); and
(xii) An ASGPR Binding Ligand described herein.

### BRIEF DESCRIPTION OF THE FIGURES

The Extracellular Protein Target Ligand (“EPTL”) is covalently bound to Linker in the ASGPR-binding extracellular protein degrader compound through the Anchor Bond (which is the chemical bond between the EPTL and either Linker B, Linker C or Linker D). This bond can be placed at any location on the ligand that does not unacceptably disrupt the ability of the EPTL to bind to the Target Extracellular Protein. The Anchor Bond is depicted on the nonlimiting examples of Extracellular Protein Target Ligands in the figures as:

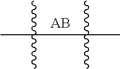 [SMILES: **]

FIG. 1A provides a non-limiting list of Extracellular Protein Targeting Ligands that target Immunoglobulin A (IgA).

FIG. 1B provides a non-limiting list of Extracellular Protein Targeting Ligands that target Immunoglobulin G (IgG).

FIG. 1C-1G provides a non-limiting list of Extracellular Protein Targeting Ligands that target Immunoglobulin E (IgE).

FIG. 1H-1M provides a non-limiting list of Extracellular Protein Targeting Ligands that target Tumor Necrosis Factor alpha (TNF-α).

FIG. 1N provides a non-limiting list of Extracellular Protein Targeting Ligands that target Interleukin-1 (IL-1).

FIG. 1O-1S provides a non-limiting list of Extracellular Protein Targeting Ligands that target Interleukin-2 (IL-2).

FIG. 1T-1W provides a non-limiting list of Extracellular Protein Targeting Ligands that target Interleukin-6 (IL-6).

FIG. 1X-1AA provides a non-limiting list of Extracellular Protein Targeting Ligands that target Interferon gamma (IFN-γ).

FIG. 1BB-1KK provides a non-limiting list of Extracellular Protein Targeting Ligands that target Vascular endothelial growth factor (VEGF).

FIG. 1LL provides a non-limiting list of Extracellular Protein Targeting Ligands that target Transforming growth factor beta (TGF-β1).

FIG. 1MM-1PP provide non-limiting list of Extracellular Protein Targeting Ligands that target proprotein convertase subtilisin kexin 9 (PCSK-9).

FIG. 1QQ-1SS provides a non-limiting list of Extracellular Protein Targeting Ligands that target Carboxypeptidase B2 (CPB2).

FIG. 1TT-1UU provides a non-limiting list of Extracellular Protein Targeting Ligands that target Cholinesterase (ChE).

FIG. 1VV-1WW provides a non-limiting list of Extracellular Protein Targeting Ligands that target C-C Motif Chemokine Ligand 2 (CCL2).

FIG. 1XX-1BBB provides a non-limiting list of Extracellular Protein Targeting Ligands that target coagulation factor VII (Factor VII).

FIG. 1CCC-1FFF provides a non-limiting list of Extracellular Protein Targeting Ligands that target coagulation factor IX (Factor IX).

FIG. 1GGG provides a non-limiting list of Extracellular Protein Targeting Ligands that target CD40 Ligand (CD40L).

FIG. 1HHH-1JJJ provides a non-limiting list of Extracellular Protein Targeting Ligands that target coagulation factor Xa (Factor Xa).

FIG. 1KKK-1MMM provides a non-limiting list of Extracellular Protein Targeting Ligands that target coagulation factor XI (Factor XI).

FIGS. 1NNN and 1OOO provides a non-limiting list of Extracellular Protein Targeting Ligands that target coagulation factor XII (Factor XII).

FIGS. 1PPP and 1QQQ provides a non-limiting list of Extracellular Protein Targeting Ligands that target coagulation factor XIII (Factor XIII).

FIG. 1RRR-1UUU provides a non-limiting list of Extracellular Protein Targeting Ligands that target fibroblast growth factor 1 (FGF1).

FIG. 1VVV-1XXX provides a non-limiting list of Extracellular Protein Targeting Ligands that target fibroblast growth factor 2 (FGF2).

FIGS. 1YYY and 1ZZZ provides a non-limiting list of Extracellular Protein Targeting Ligands that target fibronectin (FN1).

FIGS. 1AAAA and 1BBBB provides a non-limiting list of Extracellular Protein Targeting Ligands that target Interleukin-5 (IL-5).

FIG. 1CCCC provides a non-limiting list of Extracellular Protein Targeting Ligands that target Interleukin-8 (IL-8).

FIGS. 1DDDD and 1EEEE provides a non-limiting list of Extracellular Protein Targeting Ligands that target Interleukin-10 (IL-10).

FIGS. 1FFFF and 1GGGG provides a non-limiting list of Extracellular Protein Targeting Ligands that target Interleukin-21 (IL-21).

FIGS. 1HHHH and 1IIII provides a non-limiting list of Extracellular Protein Targeting Ligands that target Interleukin-22 (IL-22).

FIG. 1JJJJ-1NNNN provides a non-limiting list of Extracellular Protein Targeting Ligands that target Kallikrein 1.

FIG. 1OOOO provides a non-limiting list of Extracellular Protein Targeting Ligands that target lipoprotein lipase (LPL).

FIGS. 1PPPP and 1QQQQ provides a non-limiting list of Extracellular Protein Targeting Ligands that target matrix metalloproteinase-1 (MMP1).

FIG. 1RRRR-1DDDDD provides a non-limiting list of Extracellular Protein Targeting Ligands that target Macrophage migration inhibitory factor (MIF), also known as glycosylation-inhibiting factor (GIF), L-dopachrome isomerase, or phenylpyruvate tautomerase.

FIG. 1EEEEE-1GGGGG provides a non-limiting list of Extracellular Protein Targeting Ligands that target neutrophil elastase (NE).

FIGS. 1HHHHH and 1IIIII provides a non-limiting list of Extracellular Protein Targeting Ligands that target Prothrombin.

FIG. 1JJJJJ-1NNNNN provides a non-limiting list of Extracellular Protein Targeting Ligands that target Plasma kallikrein (KLKB1).

FIG. 1OOOOO-1SSSSS provides a non-limiting list of Extracellular Protein Targeting Ligands that target plasminogen (PLG).

FIG. 1TTTTT-1XXXXX provides a non-limiting list of Extracellular Protein Targeting Ligands that target Plasminogen activator inhibitor-1 (PAI-1), endothelial plasminogen activator inhibitor or serpin E1.

FIG. 1YYYYY-1AAAAAA provides a non-limiting list of Extracellular Protein Targeting Ligands that target phospholipases A2, for example type 1B or group 1B (PLA2, PA21B, PLA2G1B, PLA2-IB).

FIG. 1BBBBBB-1DDDDDD provides a non-limiting list of Extracellular Protein Targeting Ligands that target phospholipases A2, for example type IIA or group IIA (PLA2, PLA2A, PA2IIA, PLA2G2A, PLA2-IIA).

FIG. 1EEEEEE-1NNNNNN provides a non-limiting list of Extracellular Protein Targeting Ligands that target placental growth factor (PGF).

FIG. 1OOOOOO-IQQQQQQ provides a non-limiting list of Extracellular Protein Targeting Ligands that target plasminogen activator, tissue type (tPA, PLAT).

FIG. 1RRRRRR provides a non-limiting list of Extracellular Protein Targeting Ligands that target Transforming growth factor beta 2 (TGF-β2, TGFB2).

FIG. 1SSSSSS provides a non-limiting list of Extracellular Protein Targeting Ligands that target thrombospondin 1 (TSP1, TSP-1, THBS1).

FIG. 1TTTTTT-1XXXXXX provides a non-limiting list of Extracellular Protein Targeting Ligands that target Urokinase or Urokinase-type plasminogen activator (UPA, uPA).

FIG. 2 provides a non-limiting list of exemplary Extracellular Protein Targeting Ligands that target complement factor B.

FIGS. 3A and 3B provides a non-limiting list of exemplary Extracellular Protein Targeting Ligands that target complement factor D.

FIG. 4 provides a non-limiting list of exemplary Extracellular Protein Targeting Ligands that target complement factor H.

FIG. 5 provides a non-limiting list of exemplary Extracellular Protein Targeting Ligands that target complement component 5.

FIG. 6 provides a non-limiting list of exemplary Extracellular Protein Targeting Ligands that target TNF-alpha.

FIG. 7 provides a non-limiting list of exemplary Extracellular Protein Targeting Ligands that target factor XI.

FIG. 8 provides a non-limiting formula of a compound of the present invention.

FIGS. 9A and 9B provide a non-limiting list of Extracellular Protein Targeting Ligands that target proprotein convertase subtilisin kexin 9 (PCSK-9).

FIG. 10 provides a non-limiting list of Extracellular Protein Targeting Ligands that target prostate specific membrane antigen (PSMA).

### DETAILED DESCRIPTION OF THE INVENTION

Novel extracellular protein degraders and their pharmaceutically acceptable salts and compositions thereof that degrade a Target Extracellular Protein, for example IgG, as well as starting materials and intermediates for such extracellular protein degraders and their methods of use and manufacture are provided. These extracellular protein degraders are highly potent binders of both ASGPR and their respective extracellular protein targets. Some of the extracellular protein degraders of the present invention use high binding ASGPR Binding Ligands. This increased binding affinity for ASGPR results extracellular protein degraders with various advantages over previously known extracellular protein degraders. For example, an extracellular protein degrader of the present invention can be dosed at a lower dose, less frequently, with less side effects, and/or with increased potency when compared to other extracellular protein degraders. In some embodiments, an extracellular protein degrader that incorporates one of the high binding ASGPR ligands as described herein can be sufficiently active in the form of a monodentate compound (i.e., 1:1 extracellular protein ligand to ASGPR ligand in the therapeutic molecule). Other extracellular protein degraders described herein use ASGPR Binding Ligands with improved properties for medical treatment, for example improved selectivity, pharmacokinetics, pharmacodynamics, solubility, fewer side effects and/or improved tolerability.

In certain embodiments, the extracellular protein degrading compound degrades an immunoglobulin. The immunoglobulin degraders described herein degrade a selected immunoglobulin by covalently binding a ligand of the selected immunoglobulin to a potent ASGPR binder through selected linking groups. The immunoglobulins that can be targeted according to the present invention include but are not limited to IgA, IgG, IgD, IgE, and IgM, and mutants thereof. In certain aspects of the present invention the selected immunoglobulin degrader degrades IgG.

In certain aspects the compound of the present invention is a compound of Formula I

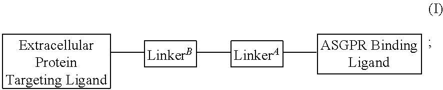

or a pharmaceutically acceptable salt thereof.

In other aspects the compound of the present invention is a compound of Formula II:

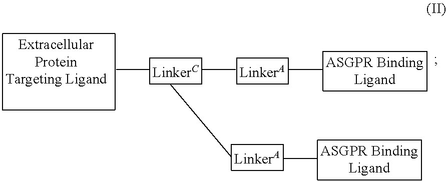

or a pharmaceutically acceptable salt thereof.

In certain embodiments, the ASGPR Binding Ligand is selected from:

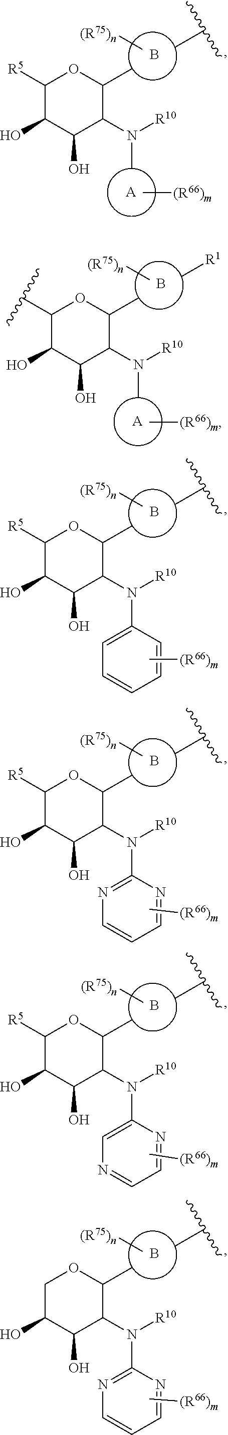

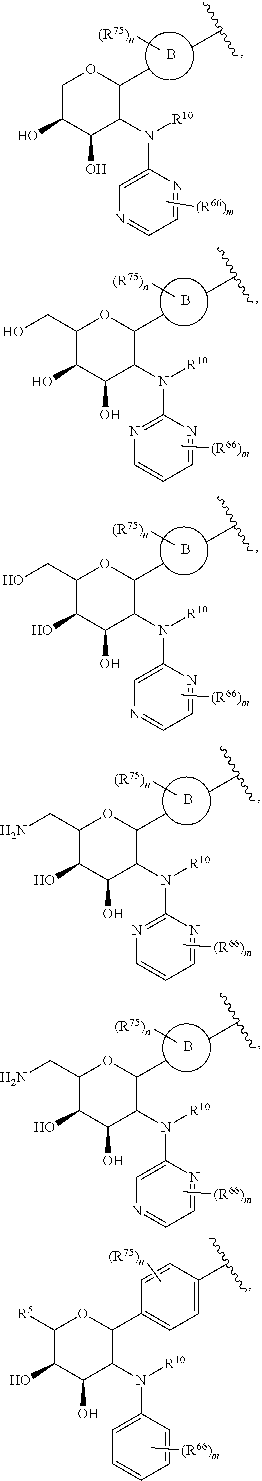

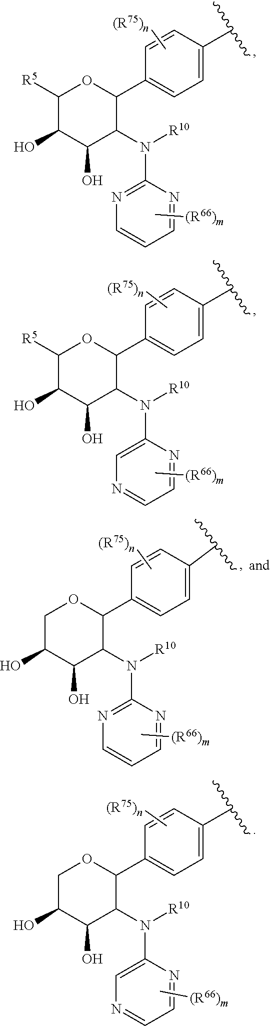

In certain embodiments, the ASGPR Binding Ligand is selected from

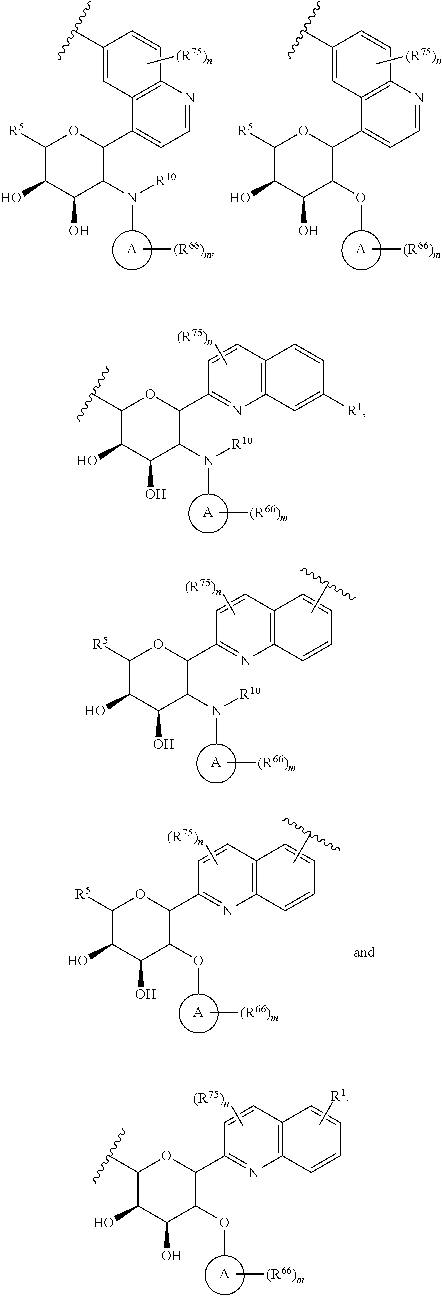

In certain embodiments, the ASGPR Binding Ligand is selected from

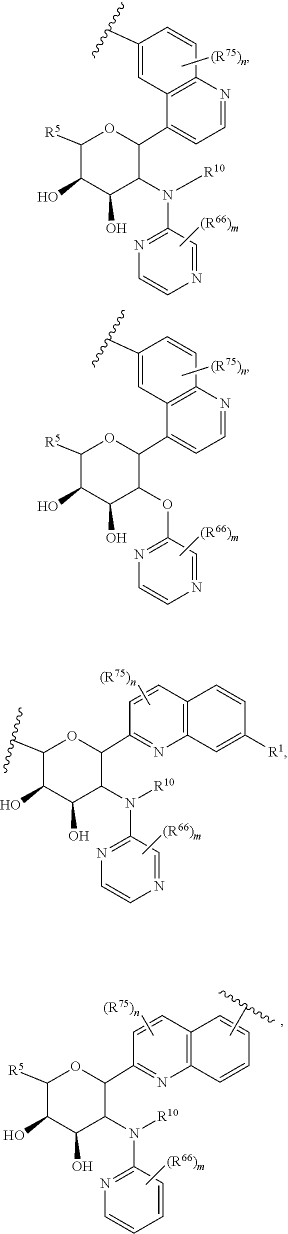

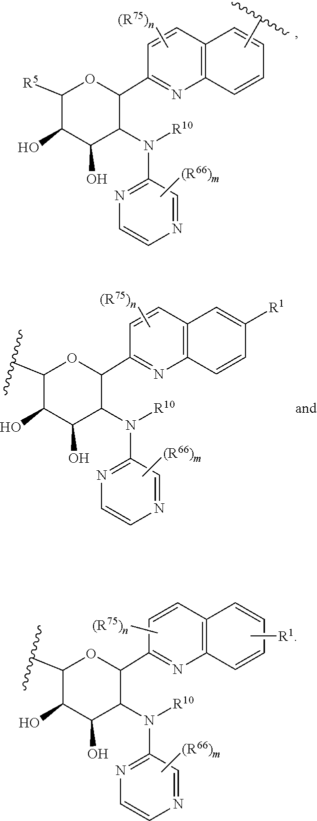

In certain embodiments, the ASGPR Binding Ligand is selected from

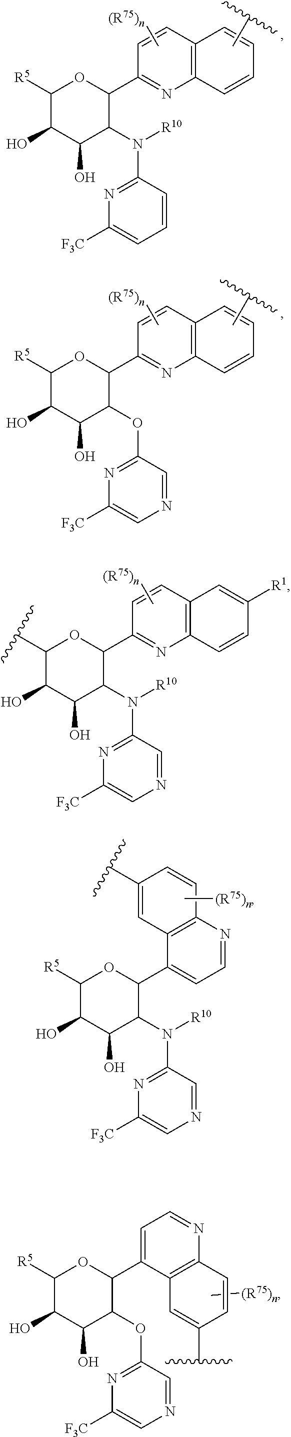

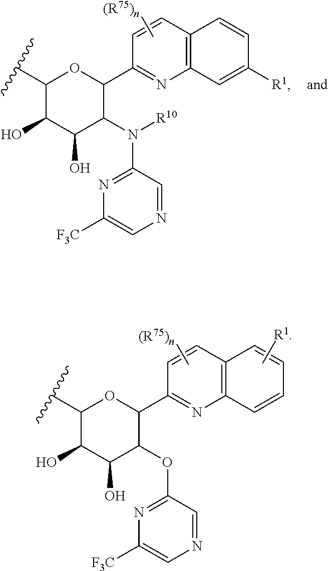

In certain embodiments, the ASGPR Binding Ligand is selected from:

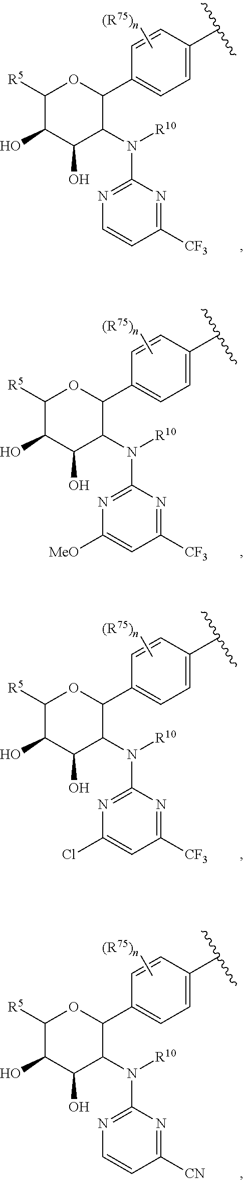

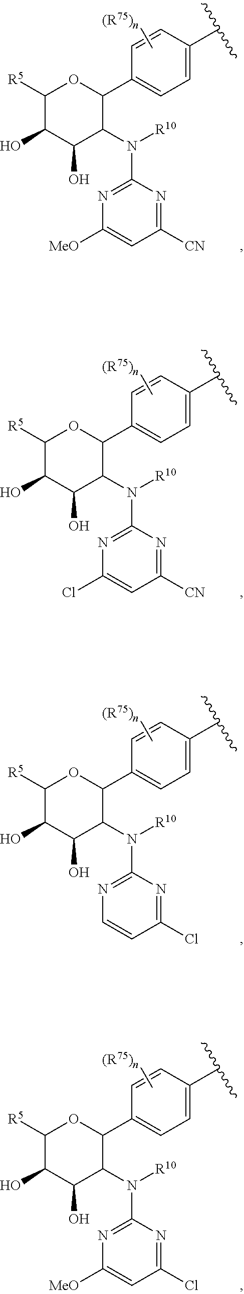

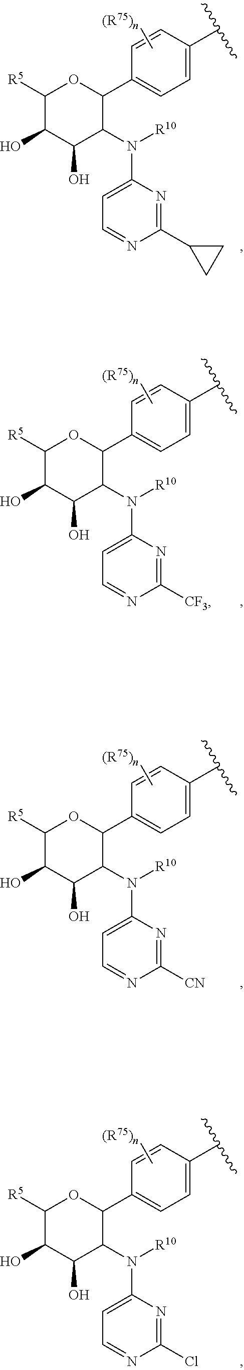

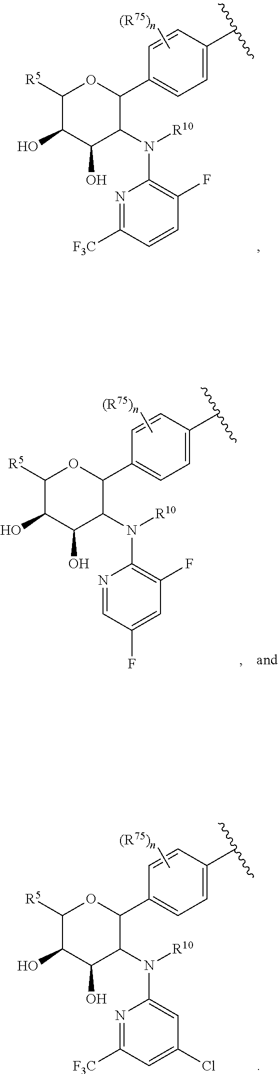

In certain embodiments, the ASGPR Binding Ligand is selected from:

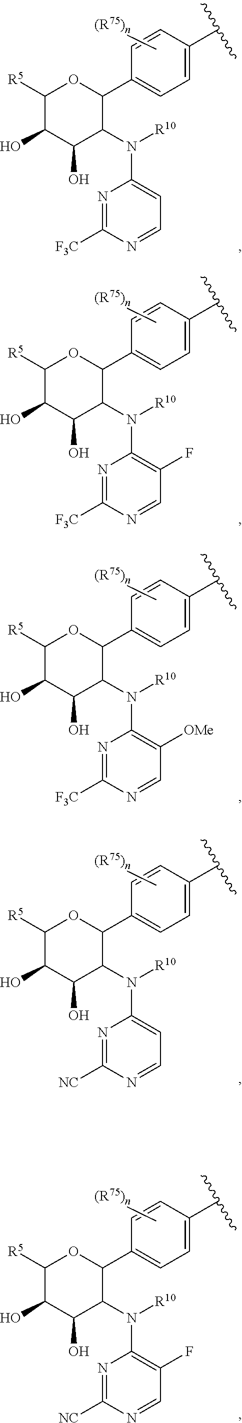

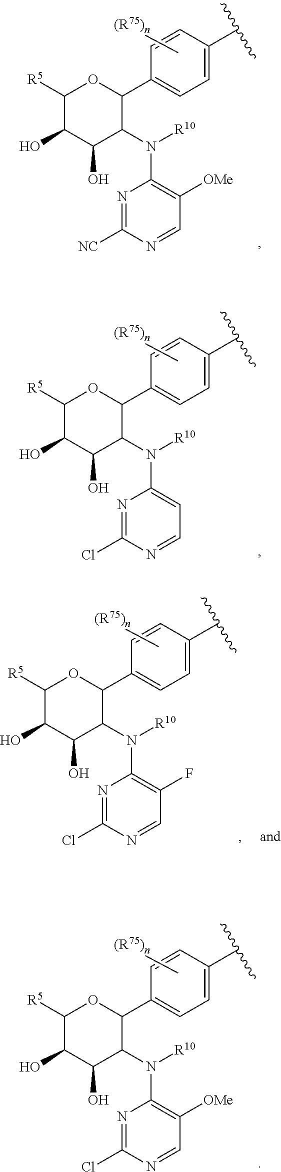

In certain embodiments, the ASGPR Binding Ligand is selected from:

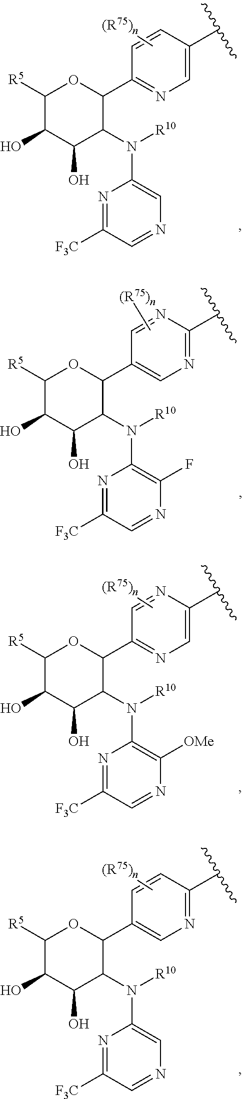

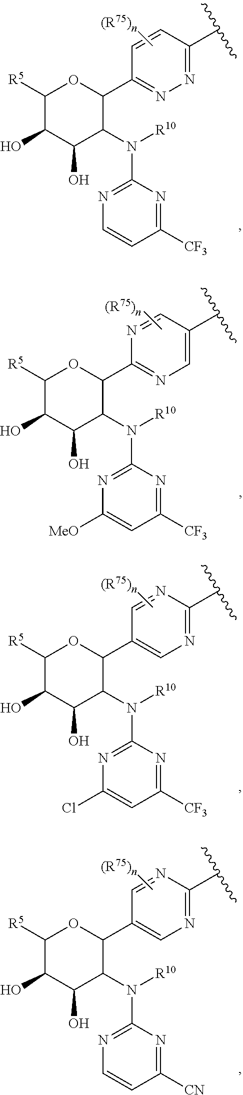

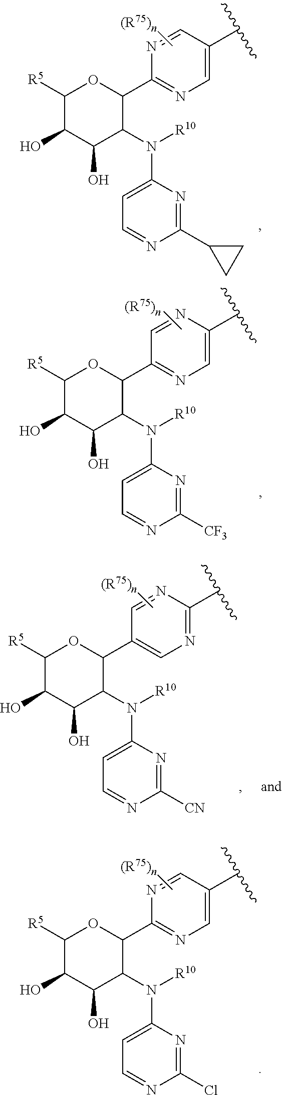

In certain embodiments, the ASGPR Binding Ligand is a compound selected from

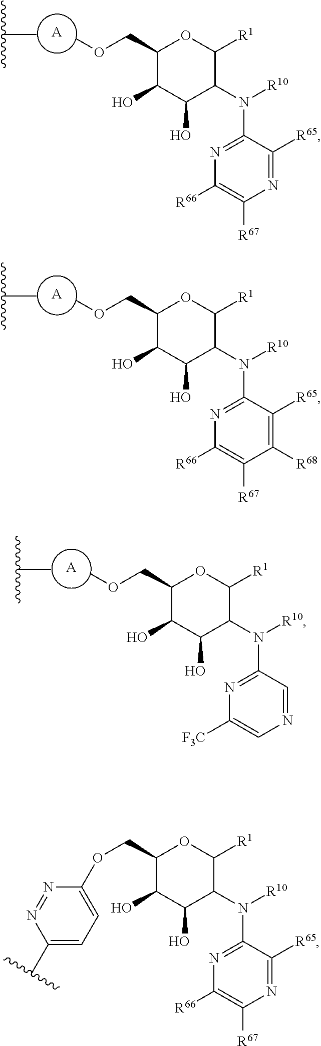

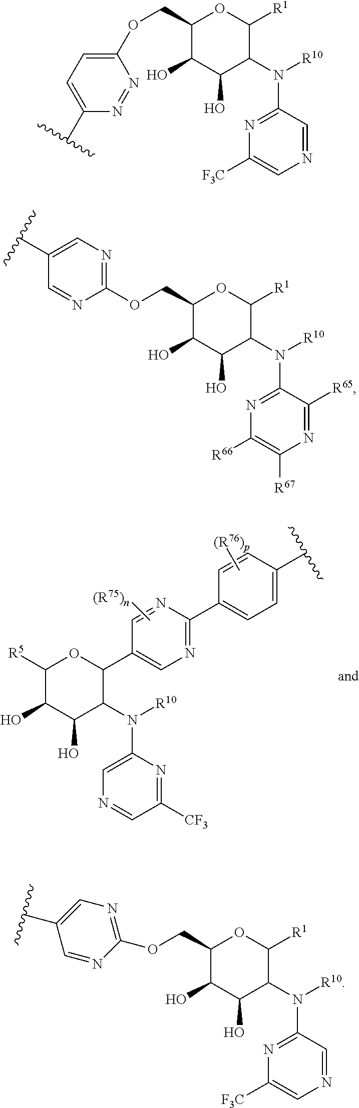

Non-limiting examples of ASGPR Binding Ligands of the present invention include:

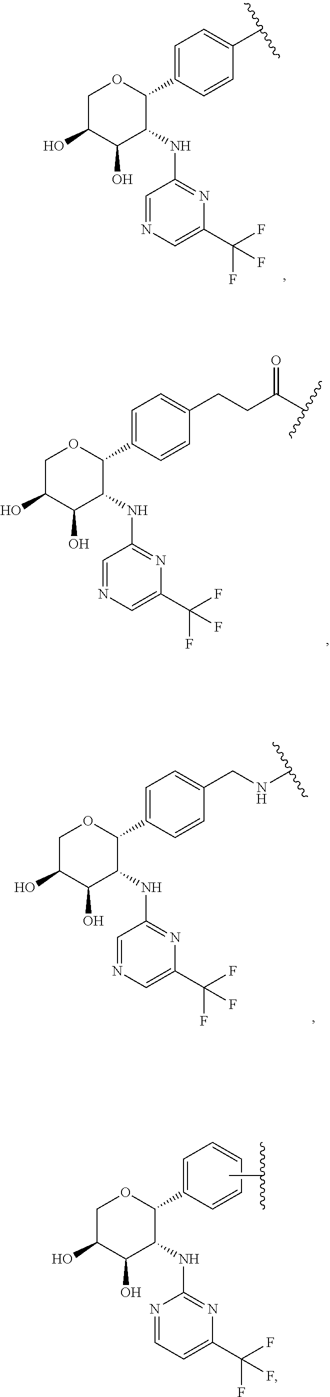

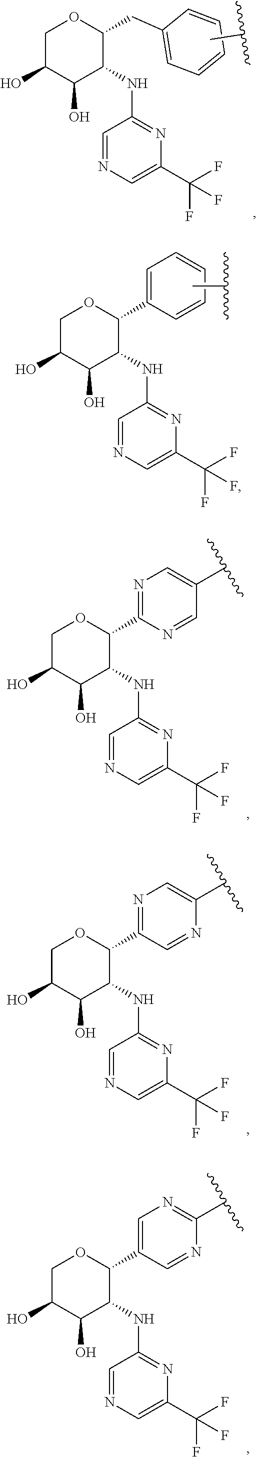

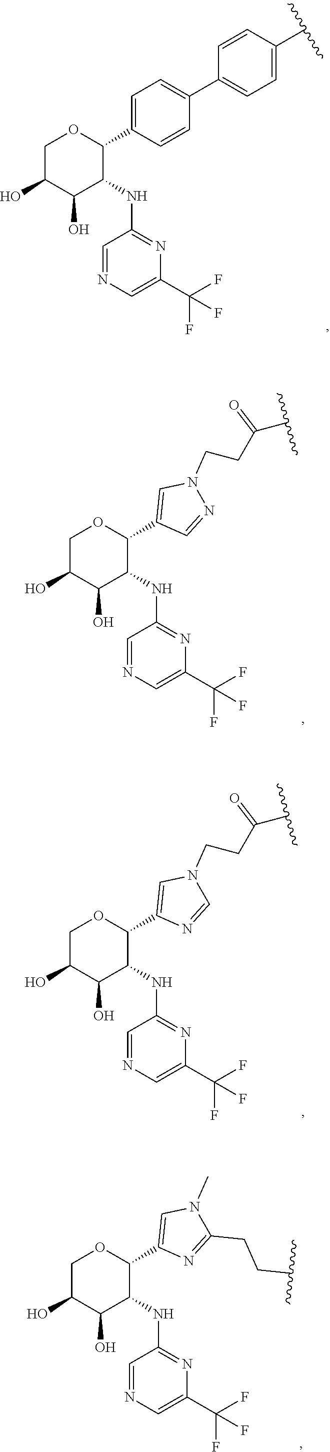 [SMILES: *c1ccc(-c2ccc([C@H]3OC[C@H](O)[C@H](O)[C@H]3Nc3cncc(C(F)(F)F)n3)cc2)cc1 ; *C(=O)CCn1cc([C@H]2OC[C@H](O)[C@H](O)[C@H]2Nc2cncc(C(F)(F)F)n2)cn1 ; *C(=O)CCn1cnc([C@H]2OC[C@H](O)[C@H](O)[C@H]2Nc2cncc(C(F)(F)F)n2)c1 ; *CCc1nc([C@H]2OC[C@H](O)[C@H](O)[C@H]2Nc2cncc(C(F)(F)F)n2)cn1C]

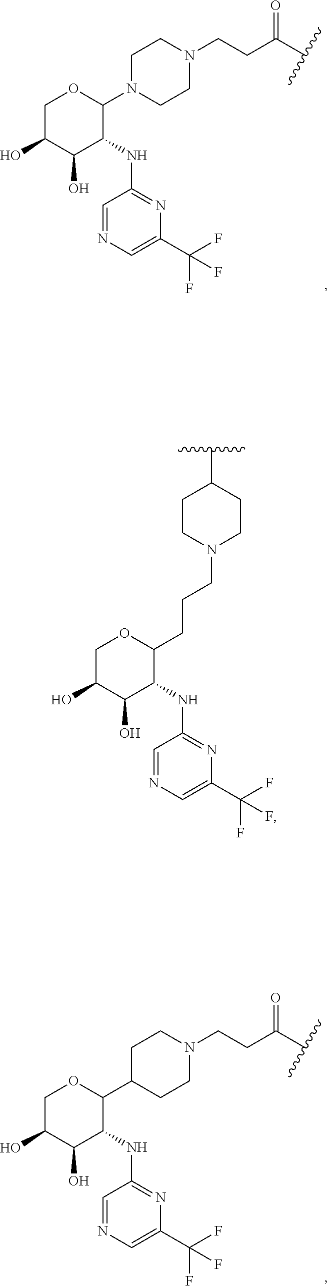 [SMILES: *C(=O)CCN1CCN(C2OC[C@H](O)[C@H](O)[C@H]2Nc2cncc(C(F)(F)F)n2)CC1 ; *C1CCN(CCCC2OC[C@H](O)[C@H](O)[C@H]2Nc2cncc(C(F)(F)F)n2)CC1 ; *C(=O)CCN1CCC(C2OC[C@H](O)[C@H](O)[C@H]2Nc2cncc(C(F)(F)F)n2)CC1]

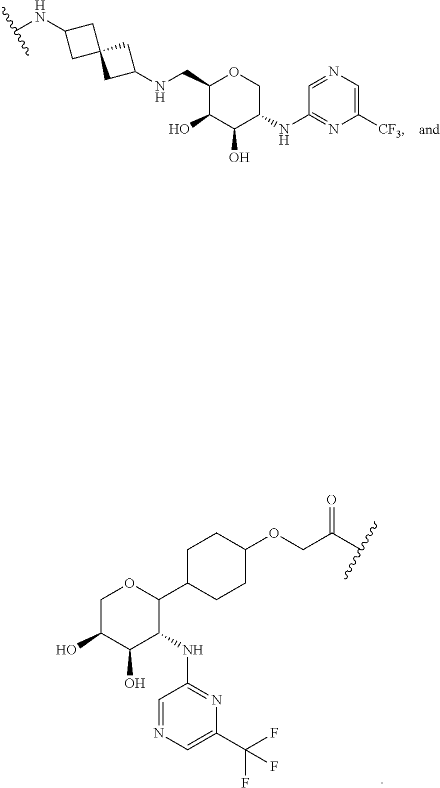

In certain embodiments, the ASGPR Binding Ligand is selected from

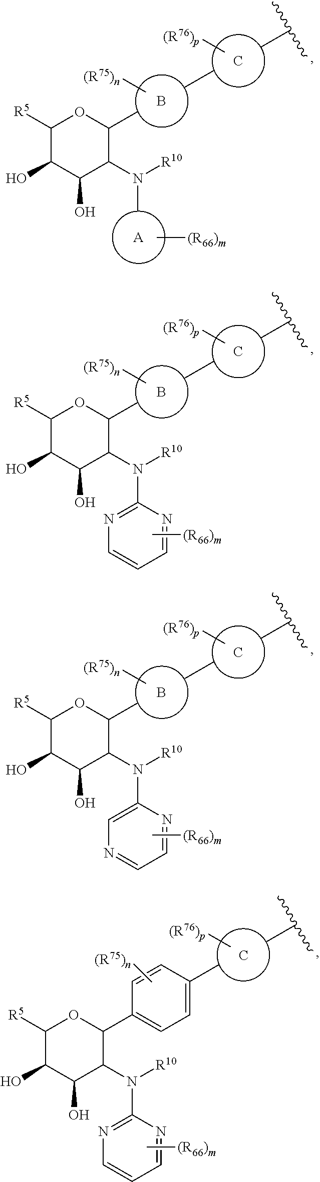

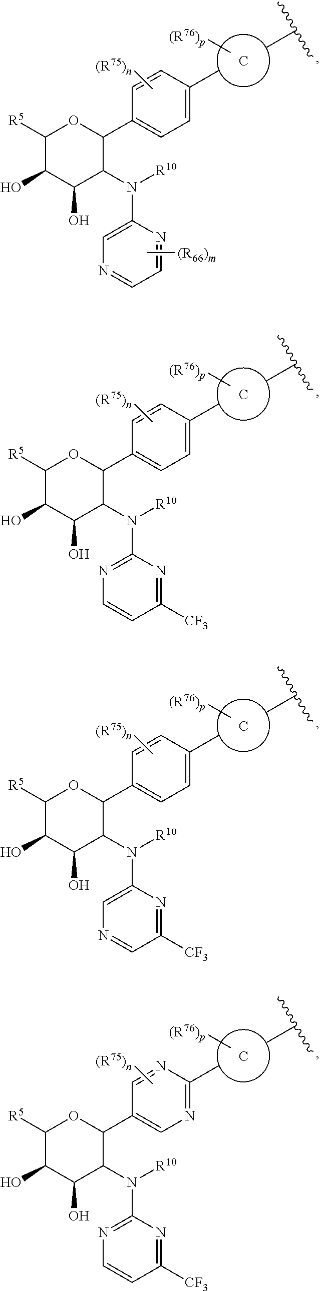

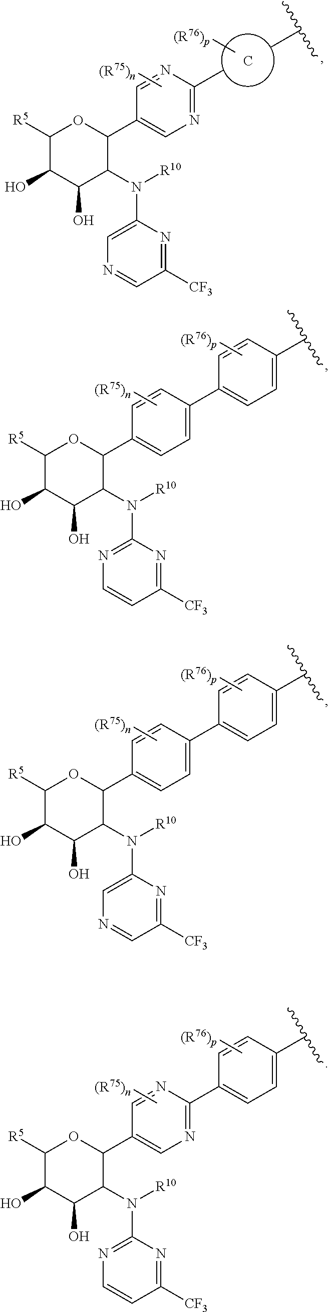

In certain embodiments, the ASGPR Binding Ligand is selected from

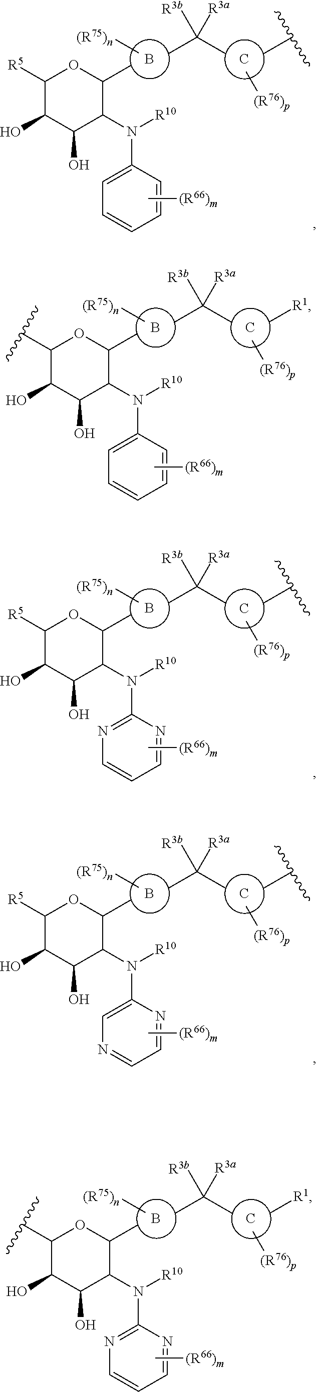

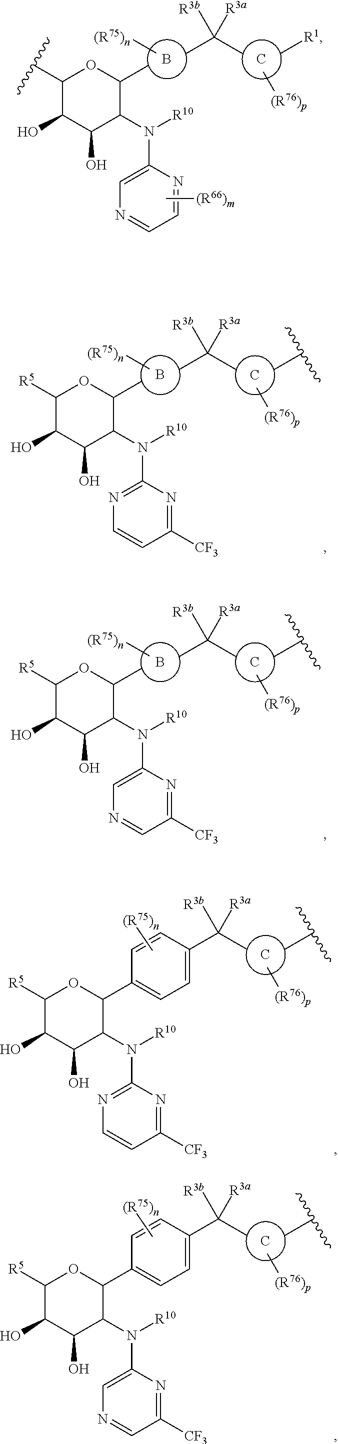

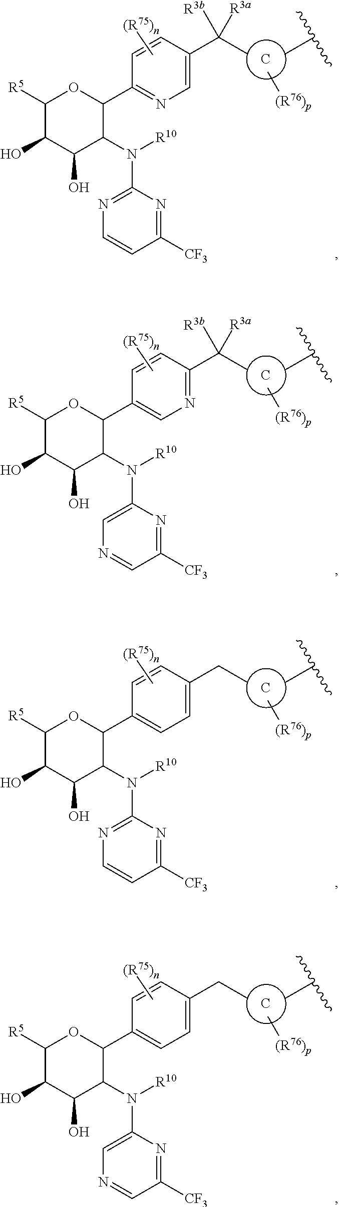

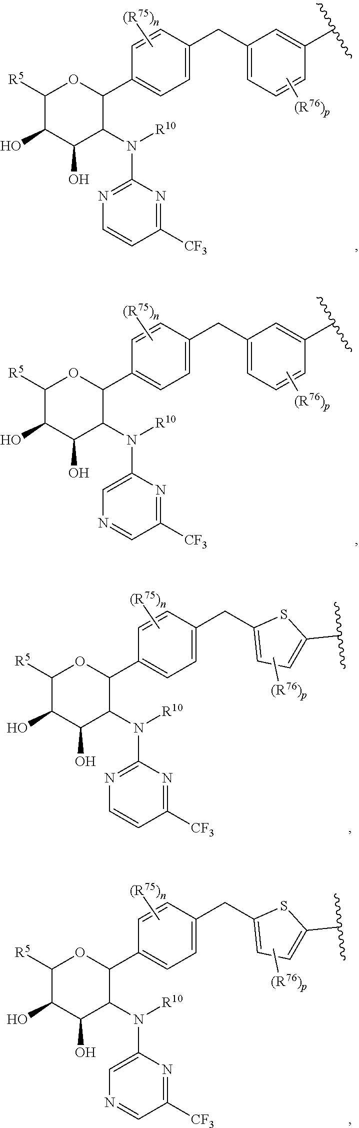

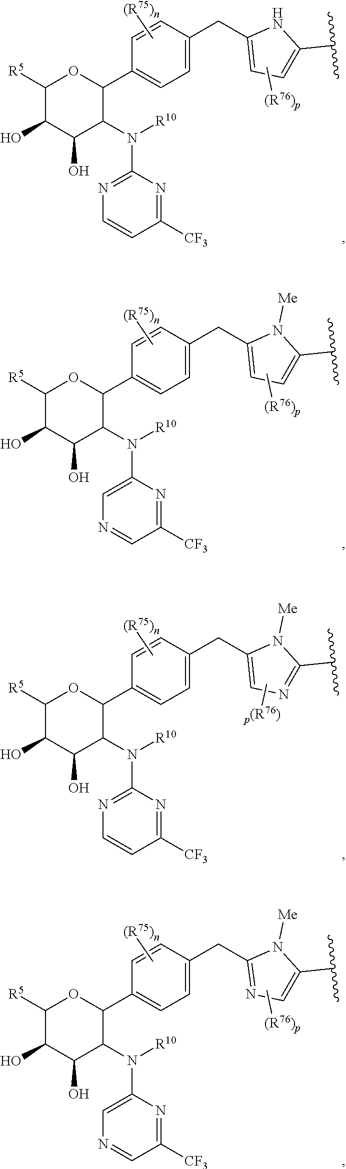

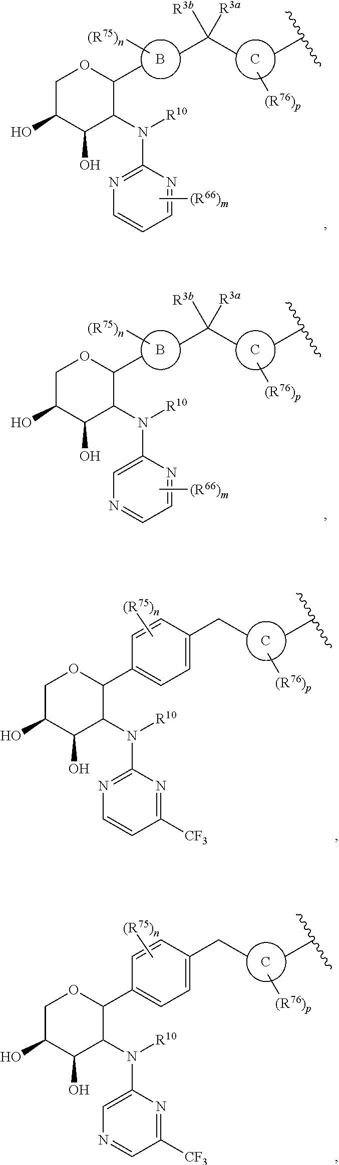

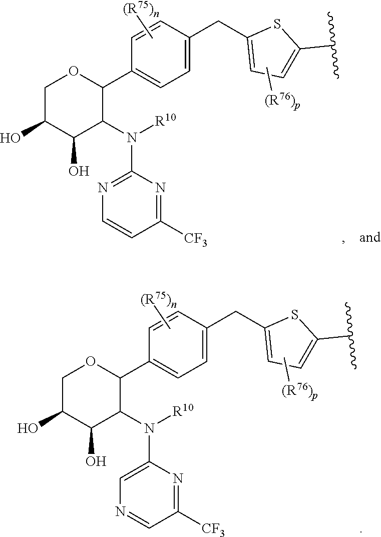

Non-limiting examples of ASGPR Binding Ligands of the present invention include:

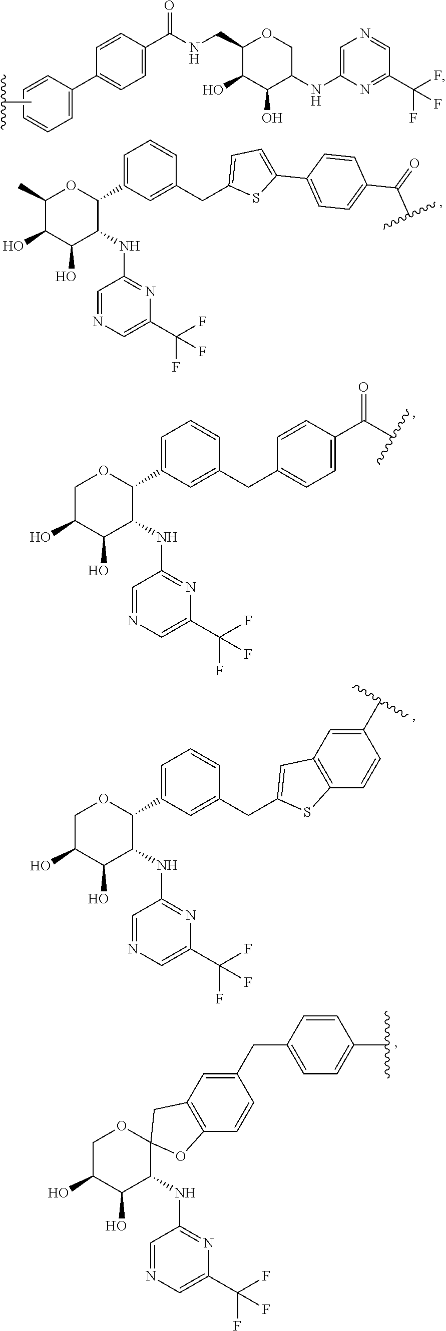

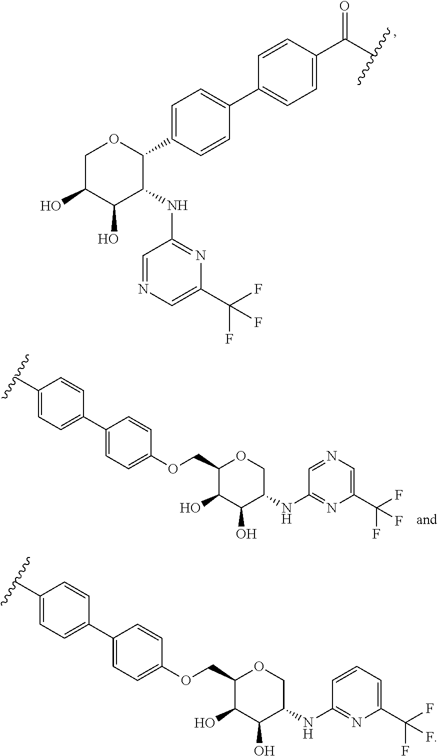 [SMILES: *C(=O)c1ccc(-c2ccc([C@H]3OC[C@H](O)[C@H](O)[C@H]3Nc3cncc(C(F)(F)F)n3)cc2)cc1 ; *c1ccc(-c2ccc(OC[C@H]3OC[C@H](Nc4cncc(C(F)(F)F)n4)[C@@H](O)[C@H]3O)cc2)cc1 ; *c1ccc(-c2ccc(OC[C@H]3OC[C@H](Nc4cccc(C(F)(F)F)n4)[C@@H](O)[C@H]3O)cc2)cc1]

Additional non-limiting examples of ASGPR Binding Ligands include:

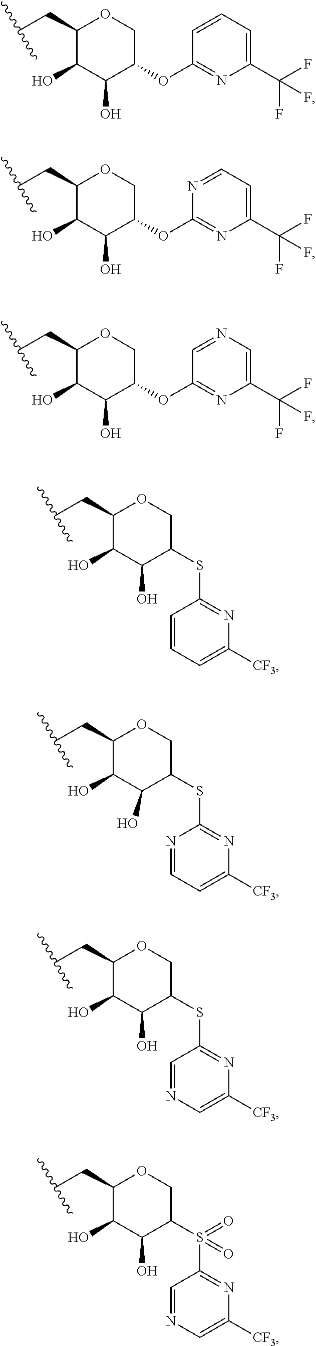

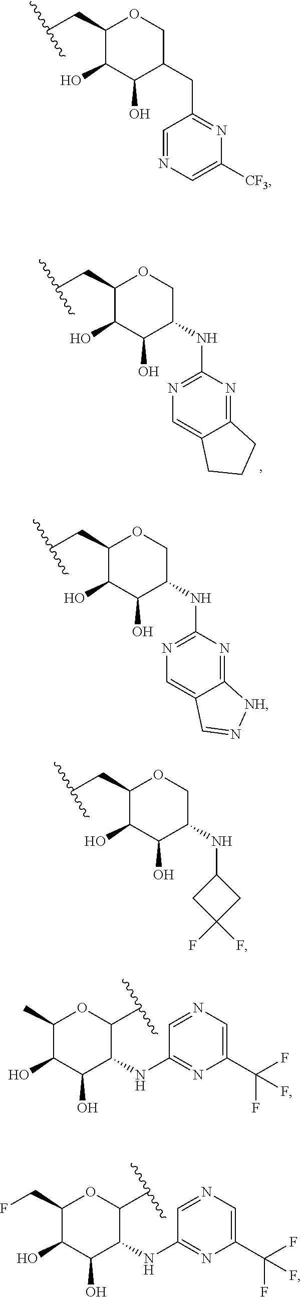 [SMILES: *C[C@H]1OCC(Cc2cncc(C(F)(F)F)n2)[C@@H](O)[C@H]1O ; *C[C@H]1OC[C@H](Nc2ncc3c(n2)CCC3)[C@@H](O)[C@H]1O ; *C[C@H]1OC[C@H](Nc2ncc3cn[nH]c3n2)[C@@H](O)[C@H]1O ; *C[C@H]1OC[C@H](NC2CC(F)(F)C2)[C@@H](O)[C@H]1O ; *C1O[C@H](C)[C@H](O)[C@H](O)[C@H]1Nc1cncc(C(F)(F)F)n1 ; *C1O[C@H](CF)[C@H](O)[C@H](O)[C@H]1Nc1cncc(C(F)(F)F)n1]

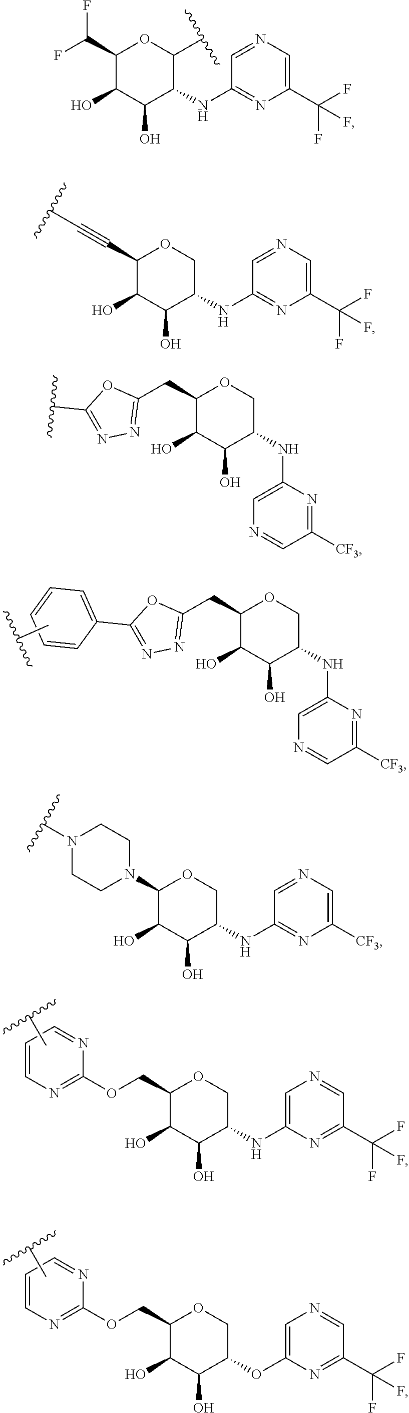

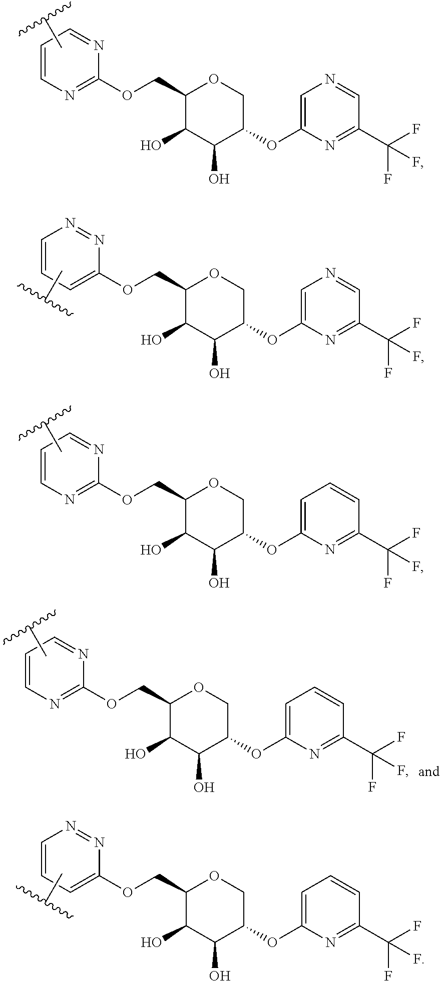

In certain embodiments, the ASGPR Binding Ligand is selected from:

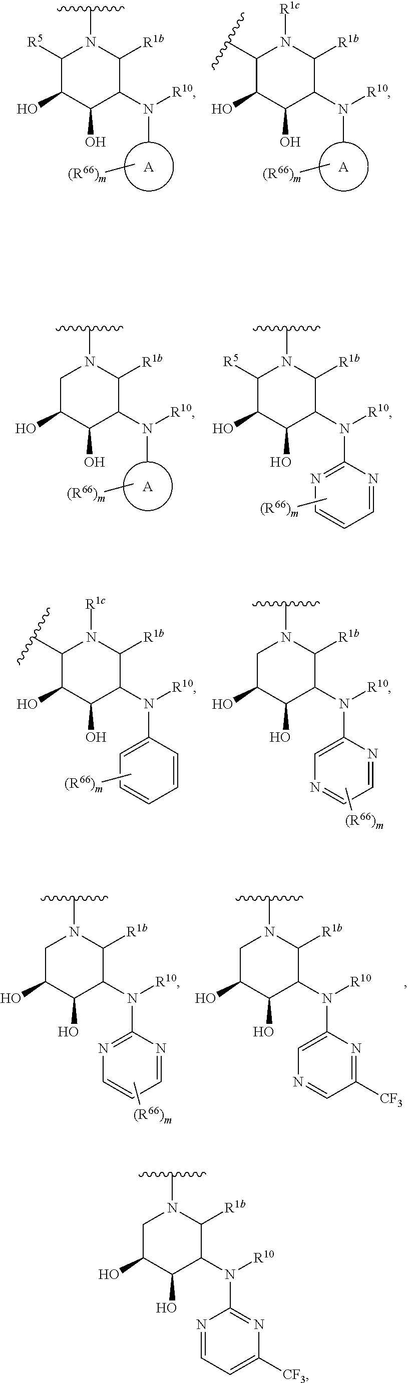

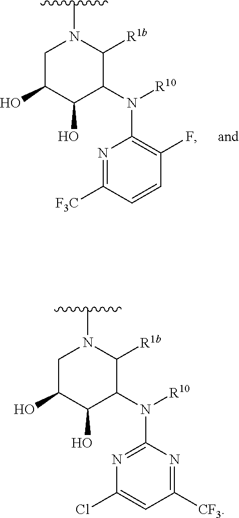

In certain embodiments, the ASGPR Binding Ligand is

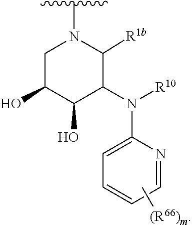

In certain embodiments, the ASGPR Binding Ligand is selected from:

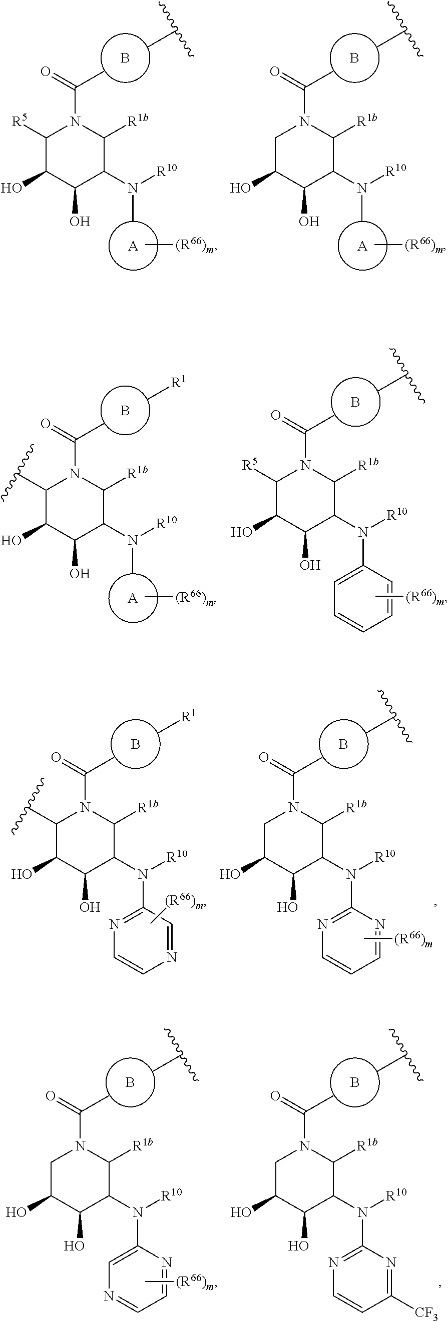

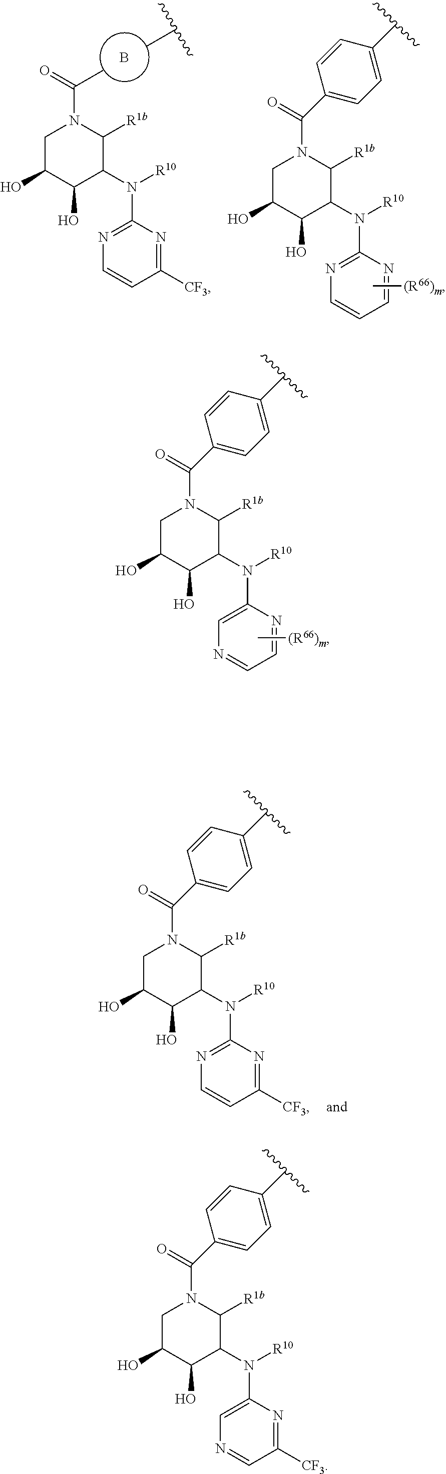

In certain embodiments, the ASGPR Binding Ligand is selected from

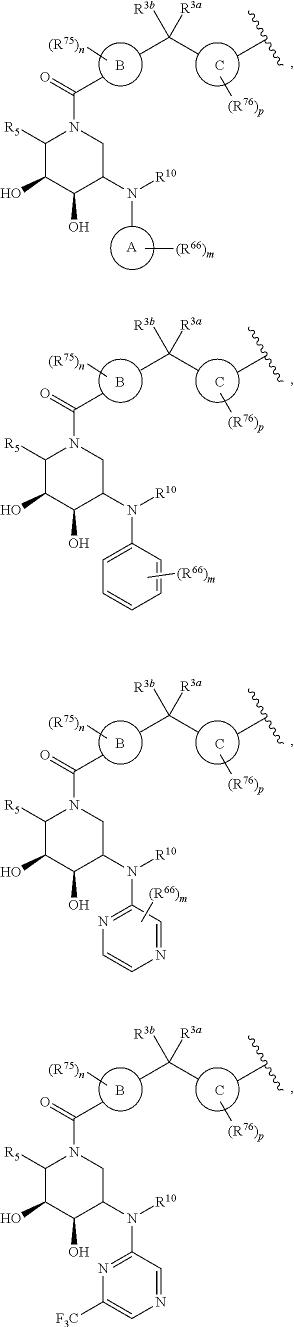

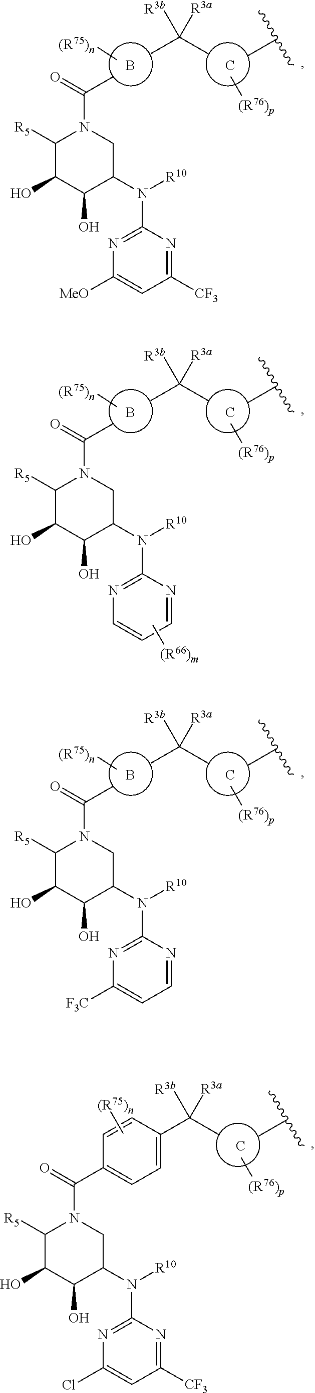

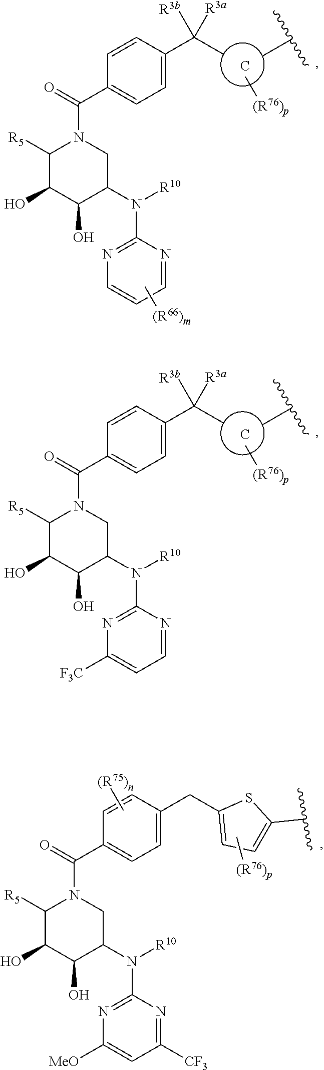

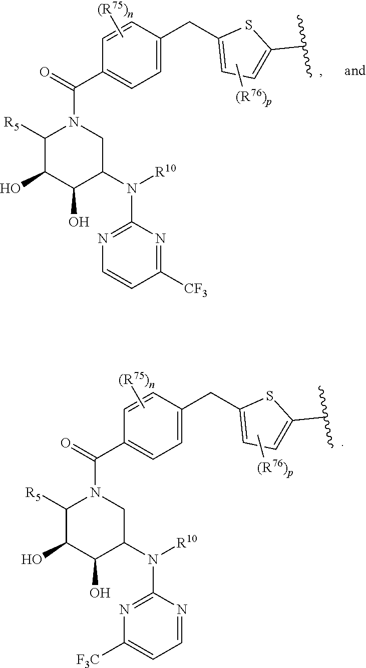

Non-limiting examples of ASGPR Binding Ligands include:

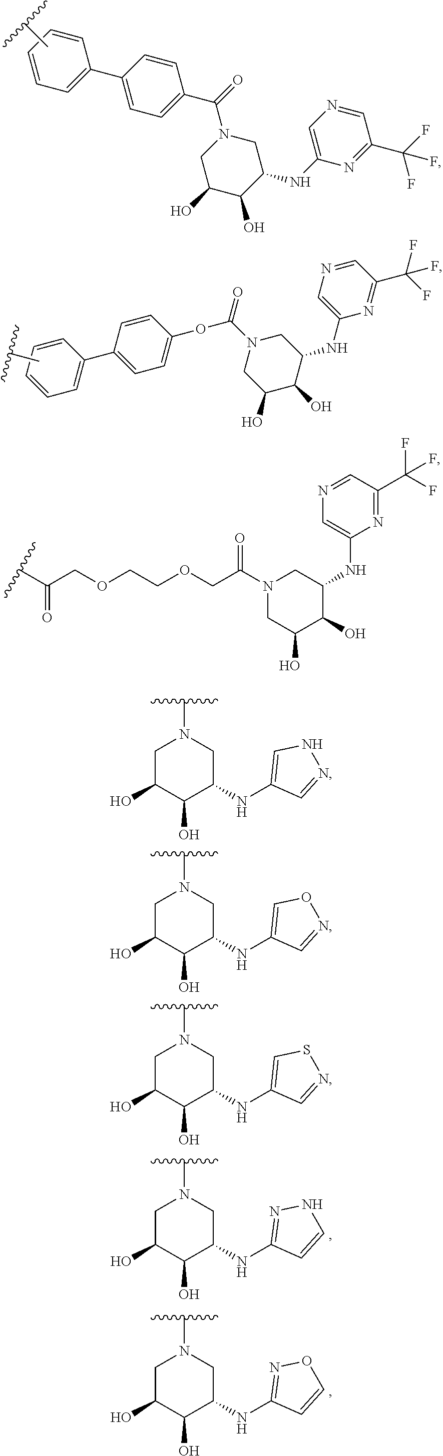

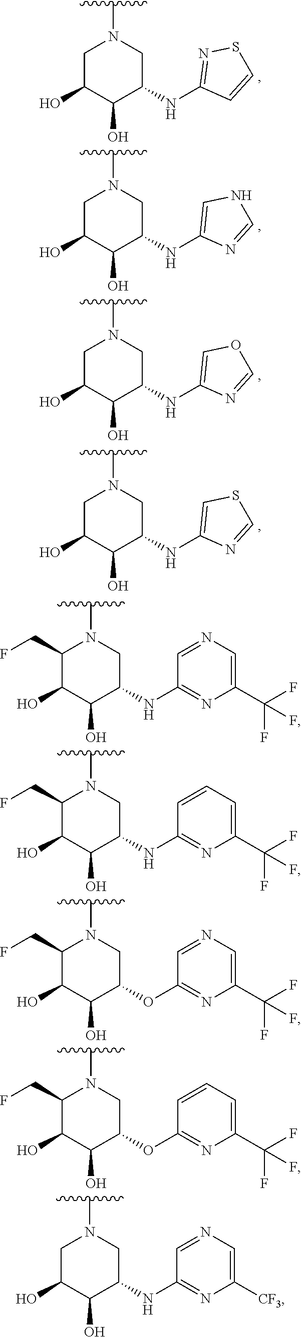

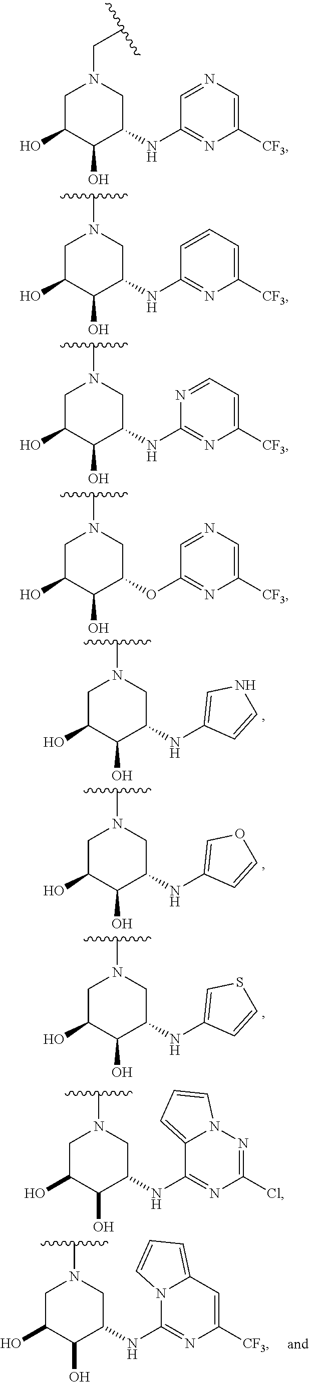

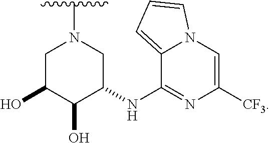 [SMILES: *N1C[C@H](Nc2nc(C(F)(F)F)cn3cccc23)[C@@H](O)[C@@H](O)C1]

In certain embodiments, the ASGPR Binding Ligand is selected from

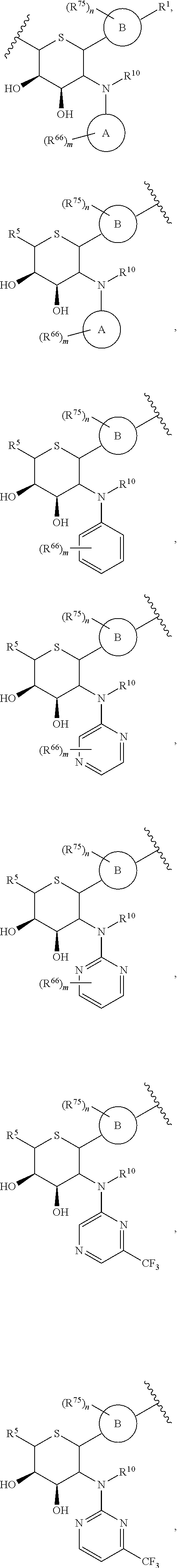

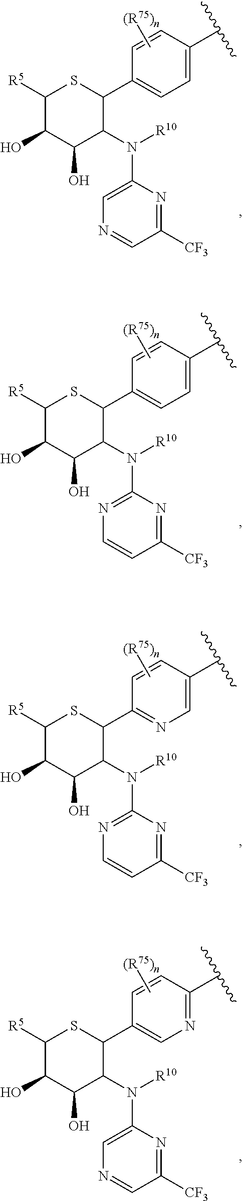

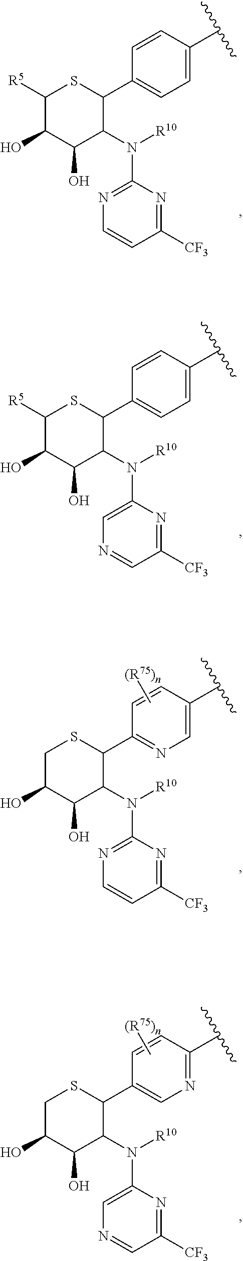

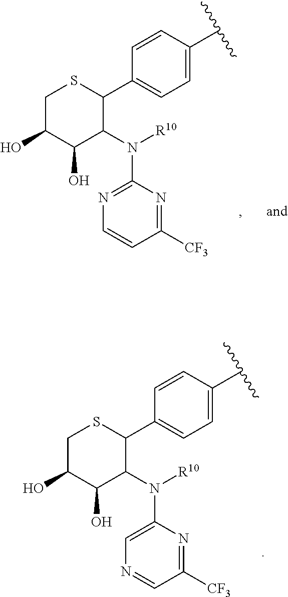

In certain embodiments, the ASGPR Binding Ligand is selected from

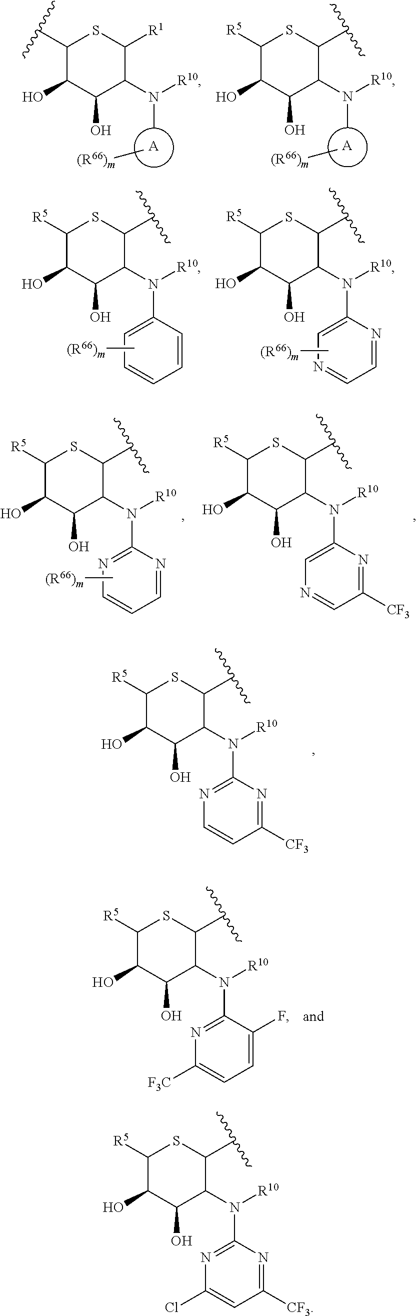

In certain embodiments, the ASGPR Binding Ligand is selected from:

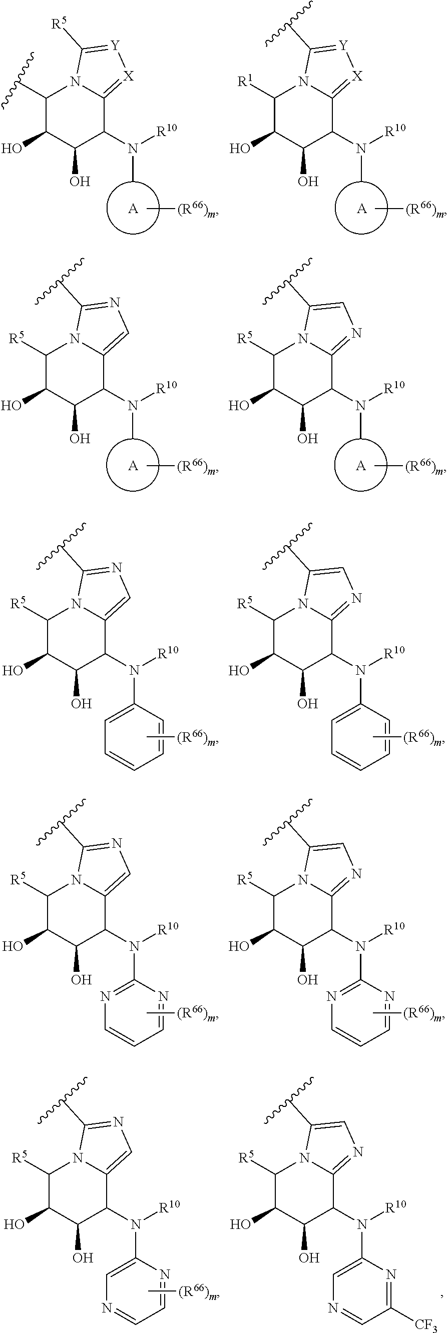

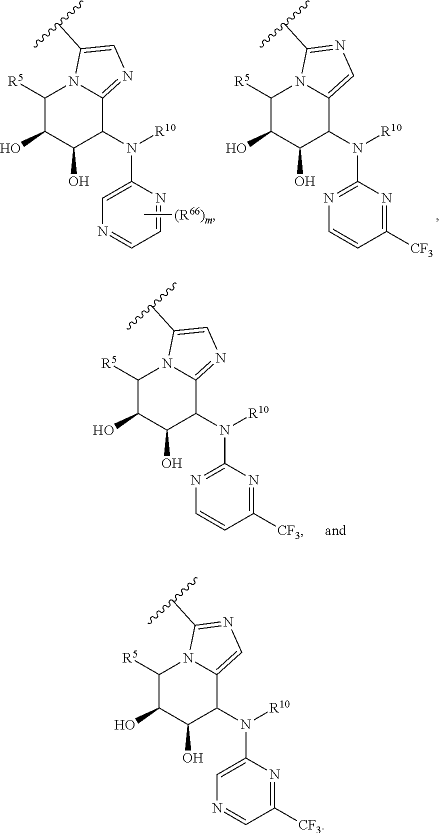

In certain embodiments, the ASGPR Binding Ligand is selected from

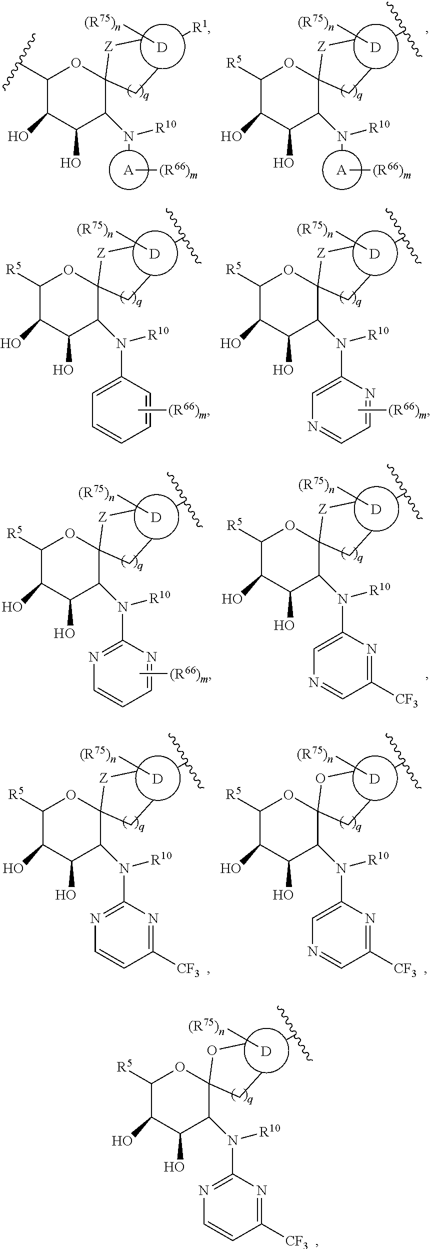

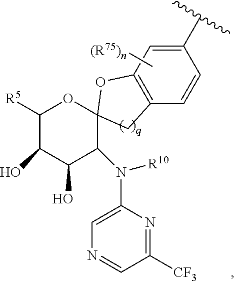

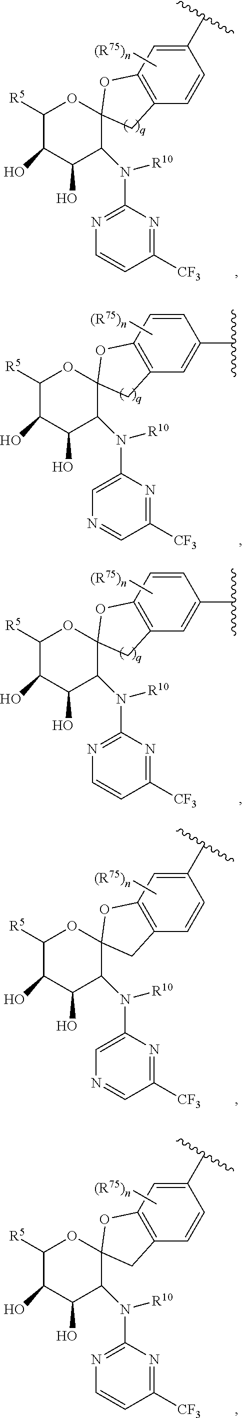

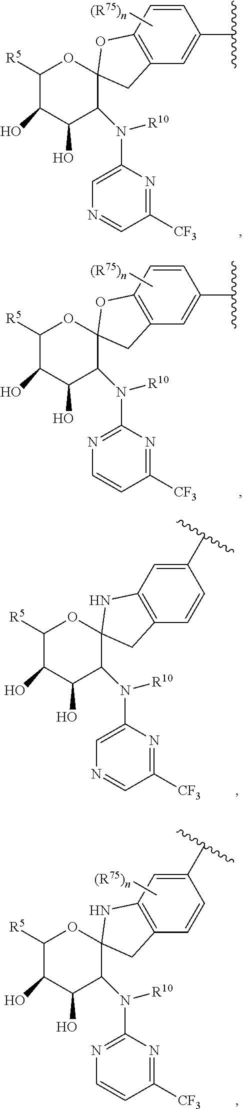

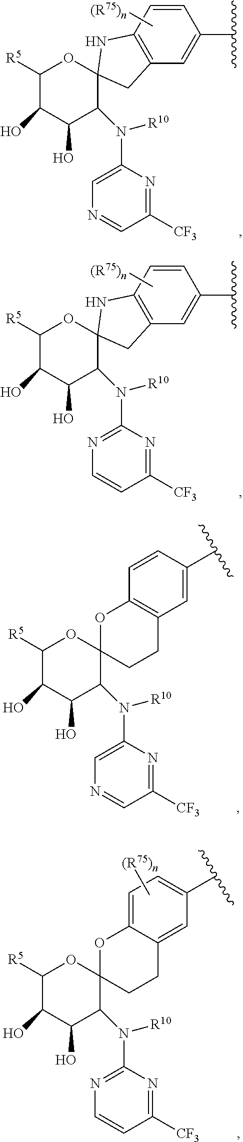

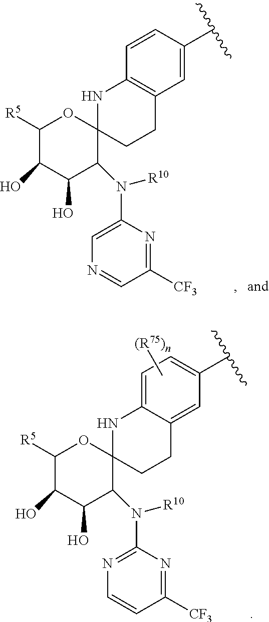

In certain embodiments, the ASGPR Binding Ligand is selected from:

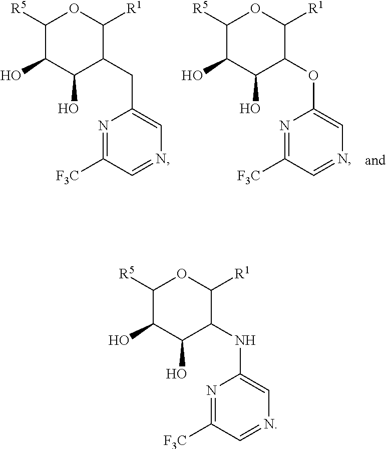

In certain embodiments, the ASGPR Binding Ligand is selected from:

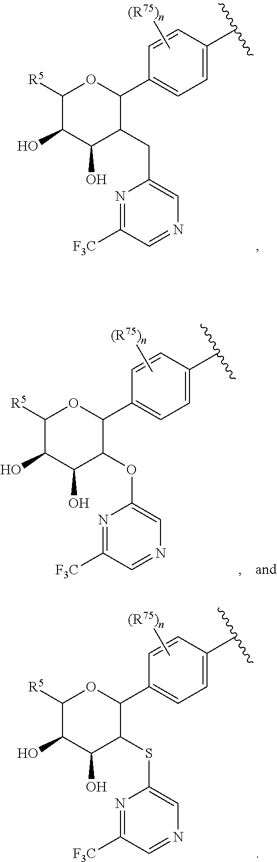

In certain embodiments, the ASGPR Binding Ligand is selected from:

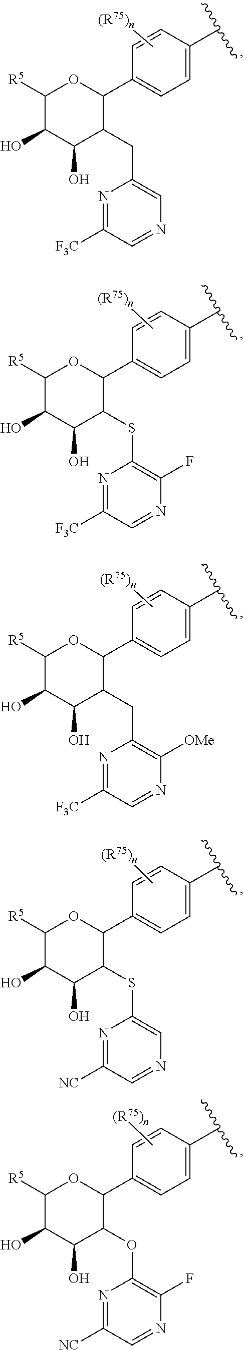

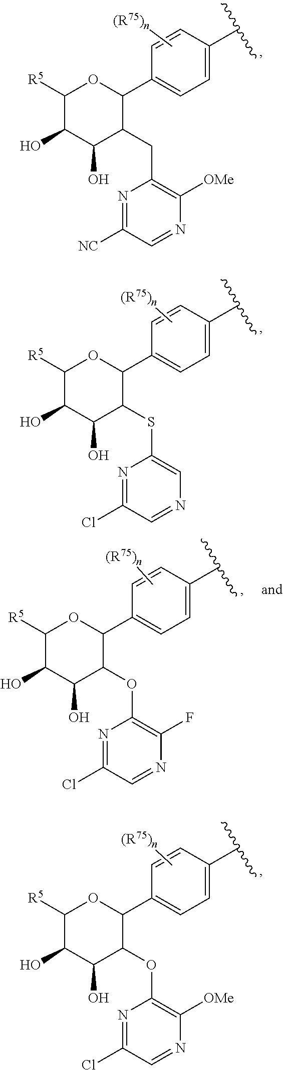

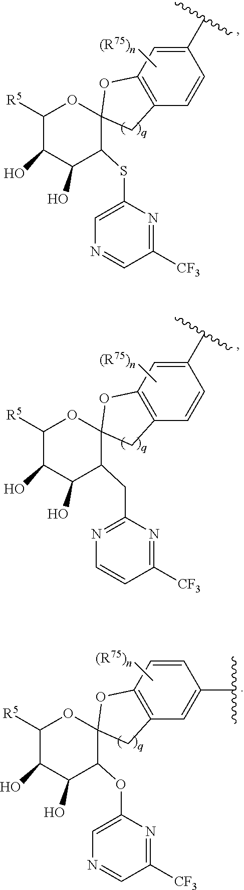

In other aspects the ASGPR Binding Ligand is selected from:

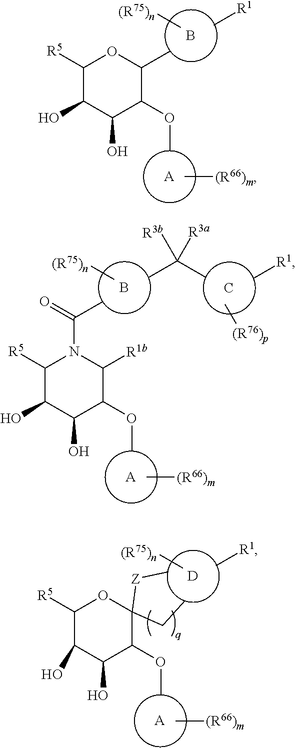

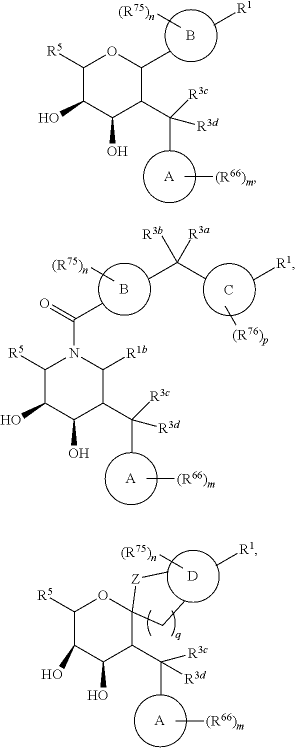

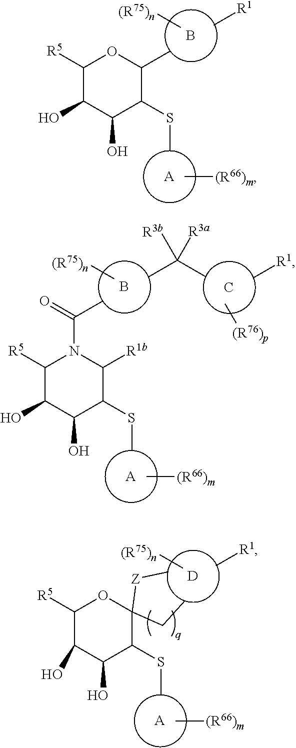

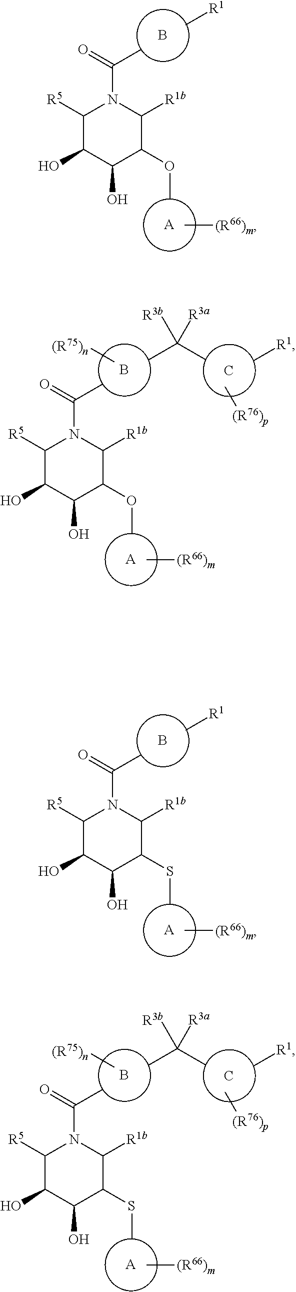

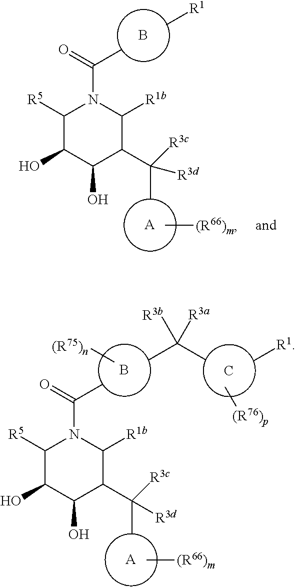

In certain embodiments the ASGPR Binding Ligand is a compound selected from:

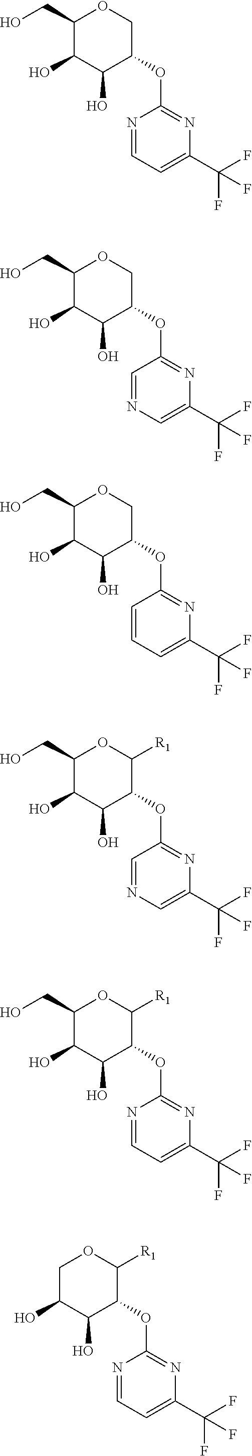

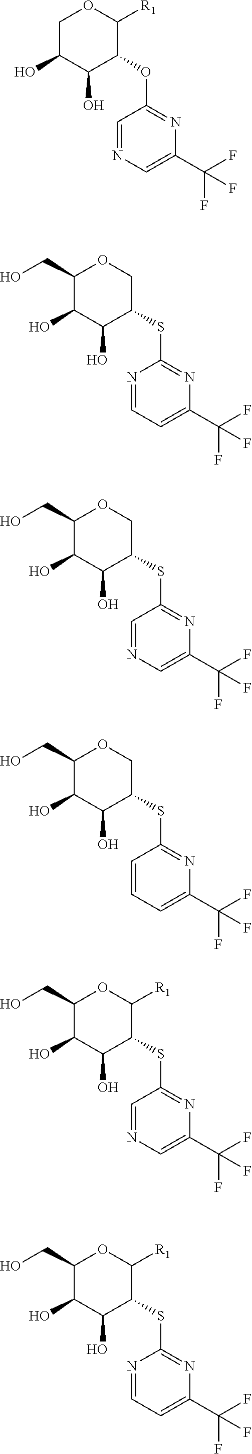

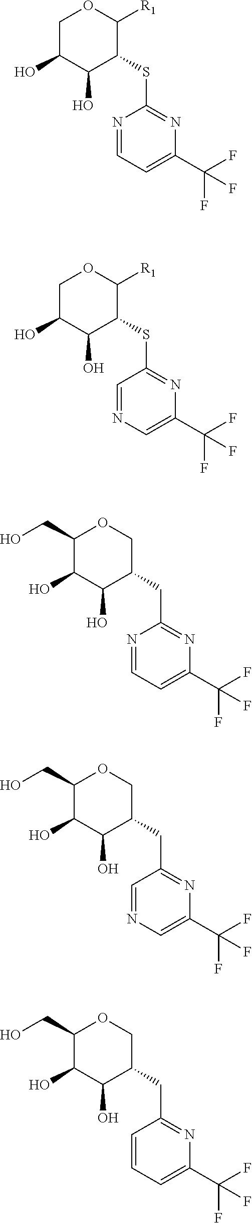

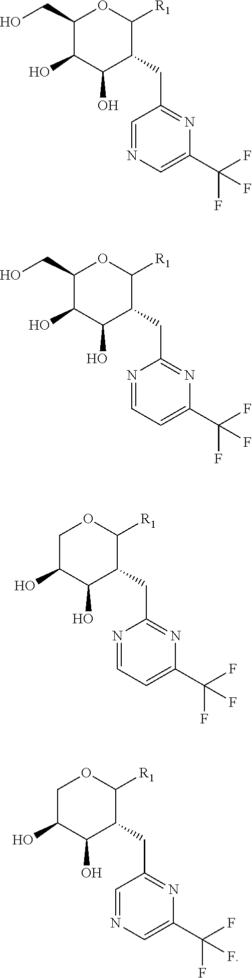

In certain embodiments the ASGPR Binding Ligand is a compound selected from:

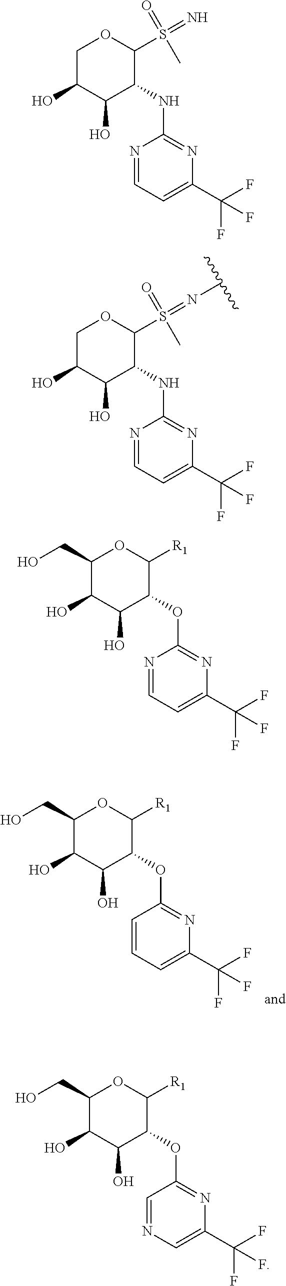

In an alternative aspect, the ASGPR Binding Ligand is a compound selected from:

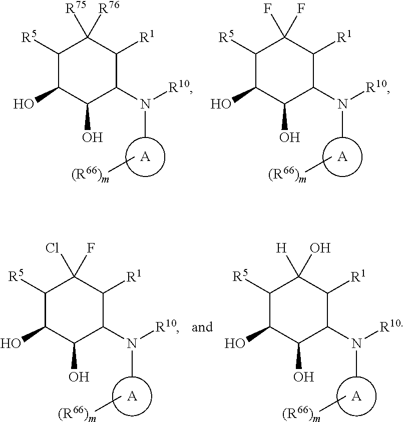

In other embodiments the ASGPR Binding Ligand is selected from:

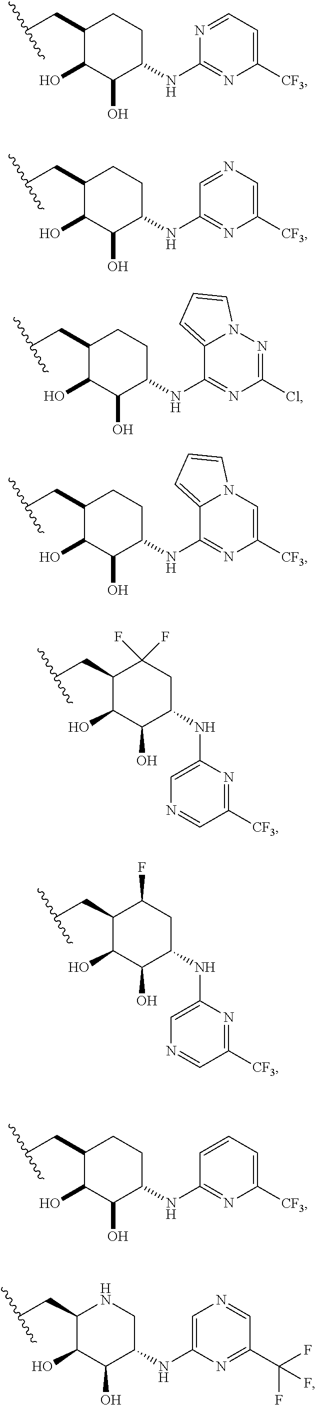

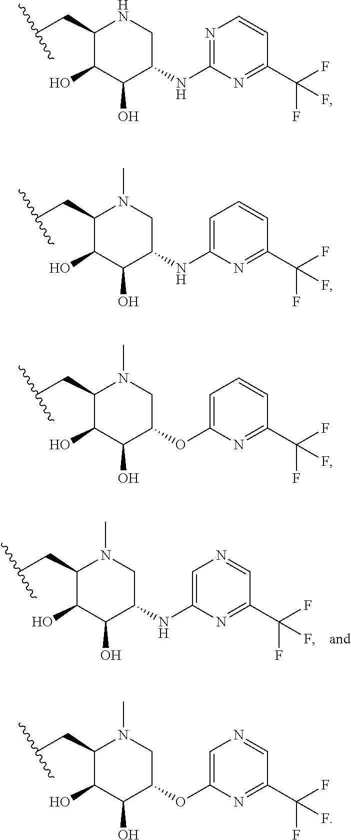

Non-limiting examples of ASGPR Binding Ligands of the present invention include:

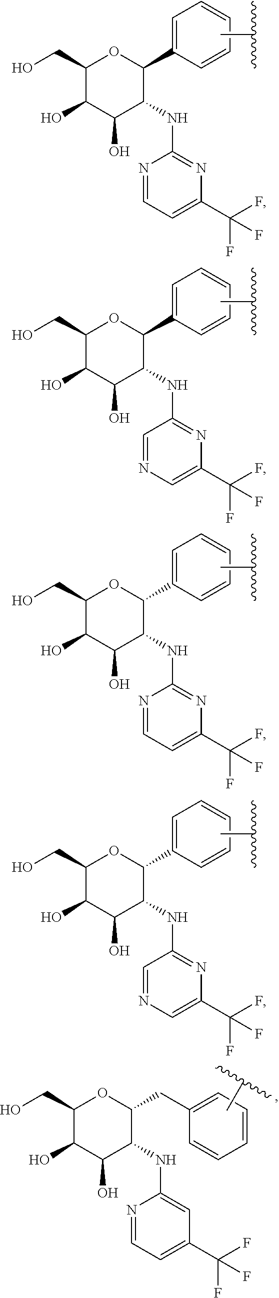

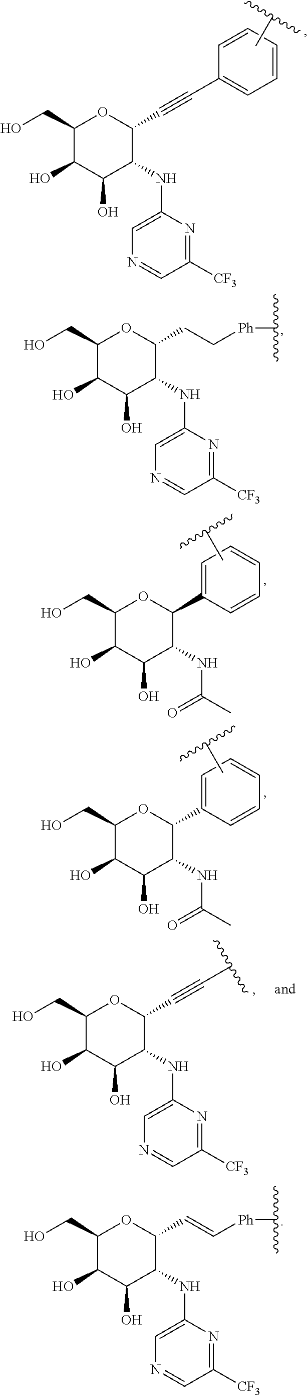

In certain embodiments the Extracellular Protein Degrading Compound of the present invention is selected from

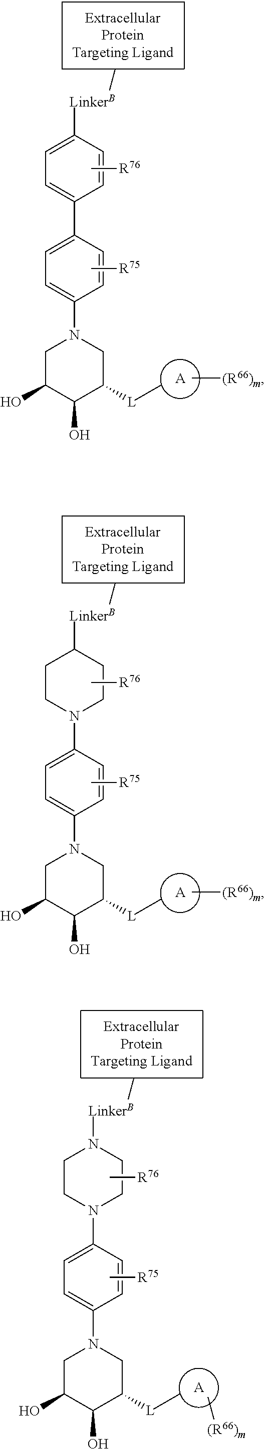

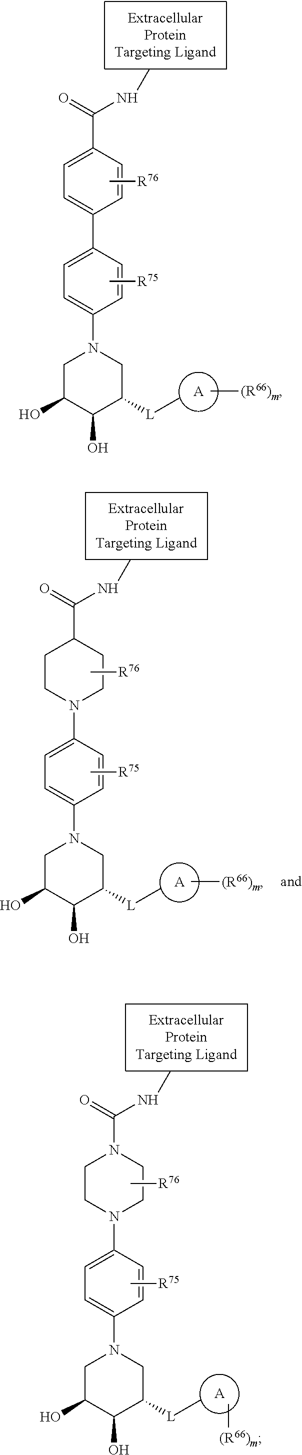

or a pharmaceutically acceptable salt thereof.

In other embodiments the Extracellular Protein Degrading Compound of the present invention is selected from

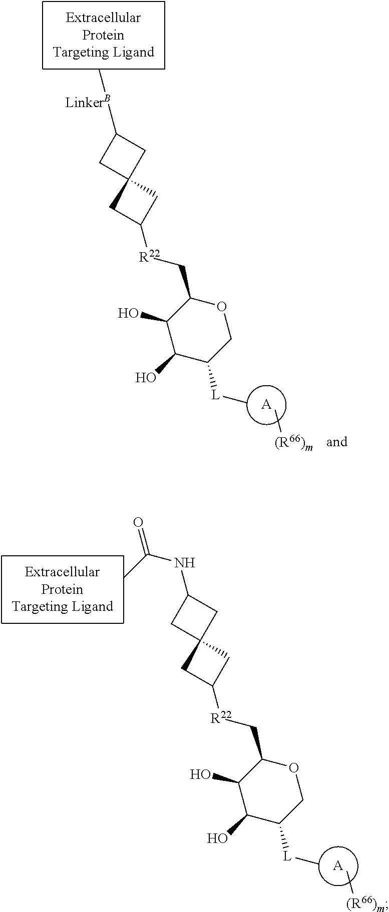

or a pharmaceutically acceptable salt thereof.

### I. Compound Terminology

Extracellular protein degraders are described using standard nomenclature. Unless defined otherwise, all technical and scientific terms used herein have the same meaning as is commonly understood by one of skill in the art to which this invention belongs.

All of the extracellular protein degraders described herein include independently the enantiomers, mixtures of enantiomers, diastereomers, tautomers, racemates and other isomers, such as rotamers, as if each is specifically described, unless otherwise indicated or otherwise excluded by context.

The terms “a” and “an” do not denote a limitation of quantity, but rather denote the presence of at least one of the referenced item. The term “or” means “and/or”. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of examples, or exemplary language (e.g., “such as”), is intended merely to better illustrate the invention and does not pose a limitation on the scope of the invention unless otherwise claimed. Unless defined otherwise, technical and scientific terms used herein have the same meaning as is commonly understood by one of skill in the art to which this invention belongs.

The present invention includes extracellular protein degraders with at least one desired isotopic substitution of an atom, at an amount above the natural abundance of the isotope, i.e., enriched.

Examples of isotopes that can be incorporated into extracellular protein degraders, of the invention include isotopes of hydrogen, carbon, nitrogen, oxygen, phosphorous, fluorine, and chlorine, such as ²H, ³H, ¹¹C, ¹³C, ¹⁴C, ¹⁵N, ¹⁷O, ¹⁸O, ¹⁸F ³¹P, ³²P, ³⁵S, ³⁶Cl, and ¹²⁵I respectively. In certain embodiments, isotopically labelled into extracellular protein degraders can be used in metabolic studies (with, for example ¹⁴C), reaction kinetic studies (with, for example ²H or ³H), detection or imaging techniques, such as positron emission tomography (PET) or single-photon emission computed tomography (SPECT) including drug or substrate tissue distribution assays, or in radioactive treatment of patients. Isotopically labeled into extracellular protein degraders of this invention and prodrugs thereof can generally be prepared by carrying out the procedures disclosed in the schemes or in the examples and preparations described below by using a readily available isotopically labeled reagent instead of a non-isotopically labeled reagent.

By way of general example and without limitation, isotopes of hydrogen, for example, deuterium (²H) and tritium (³H) may optionally be used anywhere in described structures that achieves the desired result. Alternatively, or in addition, isotopes of carbon, e.g., ¹³C and ¹⁴C, may be used. In certain embodiments, the isotopic substitution is accomplished by replacing hydrogen with a deuterium at one or more locations on the molecule to improve the performance of the drug, for example, the pharmacodynamics, pharmacokinetics, biodistribution, half-life, stability, AUC, Tₘₐₓ, Cₘₐₓ, etc. For example, the deuterium can be bound to carbon in a location of bond breakage during metabolism (an α-deuterium kinetic isotope effect) or next to or near the site of bond breakage (a β-deuterium kinetic isotope effect).

Isotopic substitutions, for example deuterium substitutions, can be partial or complete. Partial isotopic substitution means that at least one hydrogen is substituted with deuterium. In certain embodiments, the isotope is 80, 85, 90, 95 or 99% or more enriched in an isotope at any location of interest. In certain embodiments deuterium is 80, 85, 90, 95 or 99% enriched at a desired location. Unless otherwise stated, the enrichment at any point is above natural abundance, and in an embodiment is enough to alter a detectable property of the drug in a human.

The extracellular protein degraders of the present invention may form a solvate with solvents (including water). Therefore, in certain embodiments, the invention includes a solvated form of the active extracellular protein degrader. The term “solvate” refers to a molecular complex of an extracellular protein degrader of the present invention (including a salt thereof) with one or more solvent molecules. Nonlimiting examples of solvents are water, ethanol, dimethyl sulfoxide, acetone and other common organic solvents. The term “hydrate” refers to a molecular complex comprising an extracellular protein degrader of the invention and water. Pharmaceutically acceptable solvates in accordance with the invention include those wherein the solvent of crystallization may be isotopically substituted, e.g. D₂O, d₆-acetone, d₆-DMSO. A solvate can be in a liquid or solid form.

A “dosage form” means a unit of administration of an active agent. Examples of dosage forms include tablets, capsules, injections, suspensions, liquids, emulsions, implants, particles, spheres, creams, ointments, suppositories, inhalable forms, transdermal forms, buccal, sublingual, topical, gel, mucosal, implant, and the like.

“Pharmaceutical compositions” are compositions comprising at least one active agent, and at least one other substance, such as a carrier. The present invention includes pharmaceutical compositions of the described extracellular protein degraders.

“Pharmaceutical combinations” are combinations of at least two active agents which may be combined in a single dosage form or provided together in separate dosage forms.

A “pharmaceutically acceptable salt” is a derivative of the disclosed extracellular protein degrader in which the parent extracellular protein degrader is modified by making inorganic and organic, pharmaceutically acceptable, acid or base addition salts thereof. The salts of the present extracellular protein degraders can be synthesized from a parent extracellular protein degrader that contains a basic or acidic moiety by conventional chemical methods. Generally, such salts can be prepared by reacting free acid forms of these extracellular protein degraders with a stoichiometric amount of the appropriate base (such as Na, Ca, Mg, or K hydroxide, carbonate, bicarbonate, or the like), or by reacting free base forms of these extracellular protein degraders with a stoichiometric amount of the appropriate acid. Such reactions are typically carried out in water or in an organic solvent, or in a mixture of the two. Salts of the present extracellular protein degraders further include solvates of the extracellular protein degraders and of the extracellular protein degrader salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include salts which are acceptable for human consumption and the quaternary ammonium salts of the parent extracellular protein degrader formed, for example, from inorganic or organic acids. Examples, of such salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)₁₋₄—COOH, and the like, or using an acid that produces the same counterion. Lists of additional suitable salts may be found, e.g., in Remington's Pharmaceutical Sciences, 17th ed., Mack Publishing Company, Easton, Pa., p. 1418 (1985).

The term “carrier” applied to pharmaceutical compositions/combinations of the invention refers to a diluent, excipient, or vehicle with which an active extracellular protein degrader is provided.

A “pharmaceutically acceptable excipient” means an excipient that is useful in preparing a pharmaceutical composition/combination that is generally safe, acceptable for human consumption, and neither biologically nor otherwise inappropriate for administration to a host, typically a human. In certain embodiments, an excipient is used that is acceptable for veterinary use.

A “patient” or “host” or “subject” is a human or non-human animal in need of treatment or prevention of any of the disorders as specifically described herein. Typically, the host, patient, or subject is a human. A “patient” or “host” or “subject” also refers to for example, a mammal, primate (e.g., human), cow, sheep, goat, horse, dog, cat, rabbit, rat, mice, bird, and the like.

A “therapeutically effective amount” of an extracellular protein degrader, pharmaceutical composition, or combination of this invention means an amount that when administered to a host provides a therapeutic benefit such as an amelioration of symptoms or reduction or diminution of the disease itself.

In certain embodiments, the substitution of a hydrogen atom for a deuterium atom occurs within any variable group. For example, when any variable group is, or contain for example through substitution, methyl, ethyl, or methoxy, the alkyl residue may be deuterated (in nonlimiting embodiments, CDH₂, CD₂H, CD₃, CD₂CD₃, CHDCH₂D, CH₂CD₃, CHDCHD₂, OCDH₂, OCD₂H, or OCD₃ etc.). In certain other embodiments, a variable group has a or an “a” designation, which in certain embodiments can be deuterated.

The term “immunoglobulin,” typically refers to a large Y-shaped protein (e.g. an antibody) that identifies and neutralizes a foreign compound or object such as a pathogen or disease tissue.

Non-limiting examples of immunoglobulin proteins include IgA, IgD, IgE, IgG, and IgM. An immunoglobulin as used herein may also include a binding fragment as known to the skilled worker.

A dash (“-”) that is not between two letters or symbols is used to indicate a point of attachment for a substituent. For example, —(C═O)NH₂ is attached through carbon of the keto (C═O) group.

The term “substituted”, as used herein, means that any one or more hydrogens on the designated atom or group is replaced with a moiety selected from the indicated group, provided that the designated atom's normal valence is not exceeded and the resulting compound is stable.

“Alkyl” is a branched, straight chain, or cyclic saturated aliphatic hydrocarbon group. In certain embodiments, the alkyl contains from 1 to about 12 carbon atoms, more generally from 1 to about 6 carbon atoms, from 1 to about 4 carbon atoms, or from 1 to 3 carbon atoms. In certain embodiments, the alkyl contains from 1 to about 8 carbon atoms. In certain embodiments, the alkyl is C₁-C₂, C₁-C₃, C₁-C₄, C₁-C₅ or C₁-C₆. The specified ranges as used herein indicate an alkyl group which is considered to explicitly disclose as individual species each member of the range described as a unique species. For example, the term C₁-C₆ alkyl as used herein indicates a straight or branched alkyl group having from 1, 2, 3, 4, 5, or 6 carbon atoms and also a carbocyclic alkyl group of 3, 4, 5, or 6 carbon atoms and is intended to mean that each of these is described as an independent species. For example, the term C₁-C₄alkyl as used herein indicates a straight or branched alkyl group having from 1, 2, 3, or 4 carbon atoms and is intended to mean that each of these is described as an independent species. When C₀-Cₙ alkyl is used herein in conjunction with another group, for example, (C₃-C₇cycloalkyl)C₀-C₄ alkyl, or —C₀-C₄alkyl(C₃-C₇cycloalkyl), the indicated group, in this case cycloalkyl, is either directly bound by a single covalent bond (C₀alkyl), or attached by an alkyl chain in this case 1, 2, 3, or 4 carbon atoms. Alkyls can also be attached via other groups such as heteroatoms as in —O—C₀-C₄alkyl(C₃-C₇cycloalkyl). Examples of alkyl include, but are not limited to, methyl, ethyl, n-propyl, isopropyl, n-butyl, isobutyl, sec-butyl, t-butyl, n-pentyl, isopentyl, tert-pentyl, neopentyl, n-hexyl, 2-methylpentane, 3-methylpentane, 2,2-dimethylbutane, 2,3-dimethylbutane, and hexyl.

When a term is used that includes “alk” it should be understood that “cycloalkyl” or “carbocyclic” can be considered part of the definition, unless unambiguously excluded by the context. For example and without limitation, the terms alkyl, alkenyl, alkynyl, alkoxy, alkanoyl, alkenyloxy, haloalkyl, etc. can all be considered to include the cyclic forms of alkyl, unless unambiguously excluded by context.

“Alkenyl” is a branched or straight chain aliphatic hydrocarbon group having one or more carbon-carbon double bonds that may occur at a stable point along the chain. Nonlimiting examples are C₂-C₈alkenyl, C₂-C₇alkenyl, C₂-C₆alkenyl, C₂-C₅alkenyl and C₂-C₄alkenyl. The specified ranges as used herein indicate an alkenyl group having each member of the range described as an independent species, as described above for the alkyl moiety. Examples of alkenyl include, but are not limited to, ethenyl and propenyl.

“Alkynyl” is a branched or straight chain aliphatic hydrocarbon group having one or more carbon-carbon triple bonds that may occur at any stable point along the chain, for example, C₂-C₈alkynyl or C₂-C₆alkynyl. The specified ranges as used herein indicate an alkynyl group having each member of the range described as an independent species, as described above for the alkyl moiety. Examples of alkynyl include, but are not limited to, ethynyl, propynyl, 1-butynyl, 2-butynyl, 3-butynyl, 1-pentynyl, 2-pentynyl, 3-pentynyl, 4-pentynyl, 1-hexynyl, 2-hexynyl, 3-hexynyl, 4-hexynyl and 5-hexynyl.

“Alkoxy” is an alkyl group as defined above covalently bound through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. Similarly an “alkylthio” or a “thioalkyl” group is an alkyl group as defined above with the indicated number of carbon atoms covalently bound through a sulfur bridge (—S—). In certain embodiments, the alkoxy group is optionally substituted as described above.

“Haloalkyl” indicates both branched and straight-chain alkyl groups substituted with 1 or more halogen atoms, up to the maximum allowable number of halogen atoms. Examples of haloalkyl include, but are not limited to, trifluoromethyl, monofluoromethyl, difluoromethyl, 2-fluoroethyl, and penta-fluoroethyl.

“Aryl” indicates an aromatic group containing only carbon in the aromatic ring or rings. In certain embodiments, the aryl group contains 1 to 3 separate or fused rings and is 6 to 14 or 18 ring atoms, without heteroatoms as ring members. The term “aryl” includes groups where a saturated or partially unsaturated carbocycle group is fused with an aromatic ring. The term “aryl” also includes groups where a saturated or partially unsaturated heterocycle group is fused with an aromatic ring so long as the attachment point is the aromatic ring. Such compounds may include aryl rings fused to a 4 to 7 or a 5 to 7-membered saturated or partially unsaturated cyclic group that optionally contains 1, 2 or 3 heteroatoms independently selected from N, O, B, P, Si and S, to form, for example, a 3,4-methylenedioxyphenyl group. Aryl groups include, for example, phenyl and naphthyl, including 1-naphthyl and 2-naphthyl. In certain embodiments, aryl groups are pendant. An example of a pendant ring is a phenyl group substituted with a phenyl group.

The term “heterocycle” refers to saturated and partially saturated heteroatom-containing ring radicals, where the heteroatoms may be selected from N, S, and O. The term “heterocycle” includes monocyclic 3-12 membered rings, as well as bicyclic 5-16 membered ring systems (which can include fused, bridged, or spiro, bicyclic ring systems). It does not include rings containing —O—O— or —S—S— portions. Examples of saturated heterocycle groups include saturated 4- to 7-membered monocyclic groups containing 1 to 4 nitrogen atoms [e.g., pyrrolidinyl, imidazolidinyl, piperidinyl, pyrrolinyl, azetidinyl, piperazinyl, and pyrazolidinyl]; saturated 4 to 6-membered monocyclic groups containing 1 to 2 oxygen atoms and 1 to 3 nitrogen atoms [e.g., morpholinyl]; saturated 3 to 6-membered heteromonocyclic group containing 1 to 2 sulfur atoms and 1 to 3 nitrogen atoms [e.g., thiazolidinyl]. Examples of partially saturated heterocycle radicals include but are not limited to, dihydrothienyl, dihydropyranyl, dihydrofuryl, and dihydrothiazolyl. Examples of partially saturated and saturated heterocycle groups include but are not limited to, pyrrolidinyl, imidazolidinyl, piperidinyl, pyrrolinyl, pyrazolidinyl, piperazinyl, morpholinyl, tetrahydropyranyl, thiazolidinyl, dihydrothienyl, 2,3-dihydro-benzo[1,4]dioxanyl, indolinyl, isoindolinyl, dihydrobenzothienyl, dihydrobenzofuryl, isochromanyl, chromanyl, 1,2-dihydroquinolyl, 1,2,3,4-tetrahydro-isoquinolyl, 1,2,3,4-tetrahydro-quinolyl, 2,3,4,4a,9,9a-hexahydro-1H-3-aza-fluorenyl, 5,6,7-trihydro-1,2,4-triazolo[3,4-a]isoquinolyl, 3,4-dihydro-2H-benzo[1,4]oxazinyl, benzo[1,4]dioxanyl, 2,3-dihydro-1H-1λ′-benzo[d]isothiazol-6-yl, dihydropyranyl, dihydrofuryl and dihydrothiazolyl. “Bicyclic heterocycle” includes groups wherein the heterocyclic radical is fused with an aryl radical wherein the point of attachment is the heterocycle ring. “Bicyclic heterocycle” also includes heterocyclic radicals that are fused or bridged with a carbocycle radical. For example partially unsaturated condensed heterocyclic group containing 1 to 5 nitrogen atoms, for example, indoline, isoindoline, partially unsaturated condensed heterocyclic group containing 1 to 2 oxygen atoms and 1 to 3 nitrogen atoms, partially unsaturated condensed heterocyclic group containing 1 to 2 sulfur atoms and 1 to 3 nitrogen atoms, and saturated condensed heterocyclic group containing 1 to 2 oxygen or sulfur atoms.

Non-limiting examples of bicyclic heterocycles include:

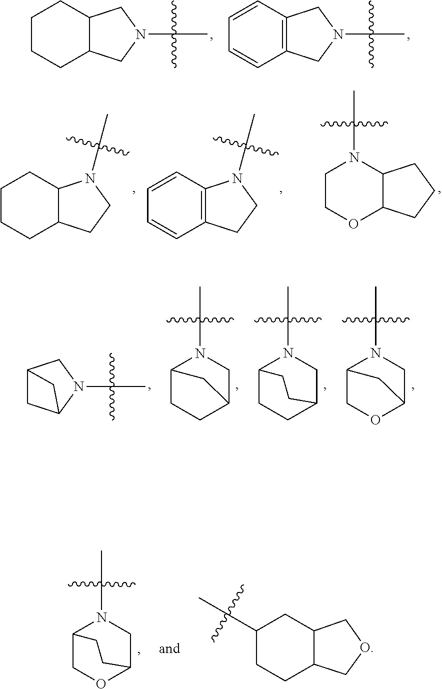

Unless otherwise drawn or clear from the context, the term “bicyclic heterocycle” includes cis and trans diastereomers. Non-limiting examples of chiral bicyclic heterocycles include:

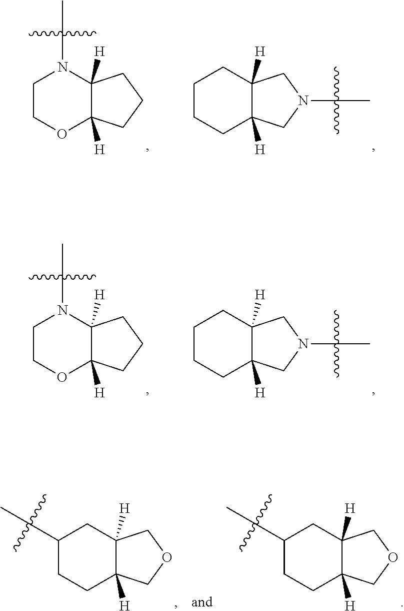 [SMILES: *N1CCO[C@H]2CCC[C@H]21 ; *N1C[C@H]2CCCC[C@H]2C1 ; *N1CCO[C@H]2CCC[C@@H]21 ; *N1C[C@@H]2CCCC[C@H]2C1 ; *C1CC[C@H]2COC[C@@H]2C1 ; *C1CC[C@H]2COC[C@H]2C1]

In certain alternative embodiments the term “heterocycle” refers to saturated and partially saturated heteroatom-containing ring radicals, where the heteroatoms may be selected from N, S, O, B, Si, and P.

“Heteroaryl” refers to a stable monocyclic, bicyclic, or multicyclic aromatic ring which contains from 1 to 3, or in some embodiments from 1, 2, or 3 heteroatoms selected from N, O, S, B, and P (and typically selected from N, O, and S) with remaining ring atoms being carbon, or a stable bicyclic or tricyclic system containing at least one 5, 6, or 7 membered aromatic ring which contains from 1 to 3, or in some embodiments from 1 to 2, heteroatoms selected from N, O, S, B or P with remaining ring atoms being carbon. In certain embodiments, the only heteroatom is nitrogen. In certain embodiments, the only heteroatom is oxygen. In certain embodiments, the only heteroatom is sulfur. Monocyclic heteroaryl groups typically have from 5 or 6 ring atoms. In some embodiments bicyclic heteroaryl groups are 8- to 10-membered heteroaryl groups, that is, groups containing 8 or 10 ring atoms in which one 5, 6, or 7-member aromatic ring is fused to a second aromatic or non-aromatic ring wherein the point of attachment is the aromatic ring. When the total number of S and O atoms in the heteroaryl group exceeds 1, these heteroatoms are not adjacent to one another. In certain embodiments, the total number of S and O atoms in the heteroaryl group is not more than 2. In another embodiment, the total number of S and O atoms in the aromatic heterocycle is not more than 1. Examples of heteroaryl groups include, but are not limited to, pyridinyl (including, for example, 2-hydroxypyridinyl), imidazolyl, imidazopyridinyl, pyrimidinyl (including, for example, 4-hydroxypyrimidinyl), pyrazolyl, triazolyl, pyrazinyl, furyl, thienyl, isoxazolyl, thiazolyl, oxadiazolyl, oxazolyl, isothiazolyl, pyrrolyl, quinolinyl, isoquinolinyl, tetrahydroisoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, triazolyl, thiadiazolyl, thiadiazolyl, furazanyl, benzofurazanyl, benzothiophenyl, benzothiazolyl, benzoxazolyl, quinazolinyl, quinoxalinyl, naphthyridinyl, tetrahydrofuranyl, and furopyridinyl. Heteroaryl groups are optionally substituted independently with one or more substituents described herein. “Heteroaryloxy” is a heteroaryl group as described bound to the group it substituted via an oxygen, —O—, linker.

“Heteroarylalkyl” is an alkyl group as described herein substituted with a heteroaryl group as described herein.

“Arylalkyl” is an alkyl group as described herein substituted with an aryl group as described herein.

“Heterocycloalkyl” is an alkyl group as described herein substituted with a heterocyclo group as described herein.

In certain embodiments, when compounds are “optionally substituted” they may be substituted as allowed by valence by groups selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, cyano, nitro, C(O)R³,

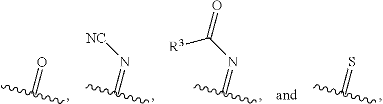

wherein the optional substituent is selected such that a stable compound results. For example

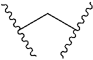 [SMILES: *C*]

could be substituted with 1 or 2 groups independently selected from alkyl, alkenyl, alkynyl, haloalkyl, —OR⁶, F, Cl, Br, I, —NR⁶R⁷, cyano, nitro, C(O)R³ so long as a stable compound results but only one group selected from,

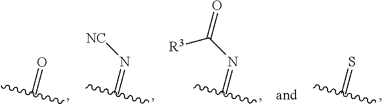

so long as a stable compound results.

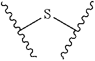 [SMILES: *S*]

on the other hand could only be substituted with 1 or 2 groups selected from

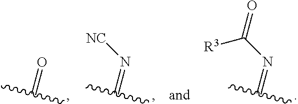

Non-limiting examples of optionally substituted CH₂ groups include:

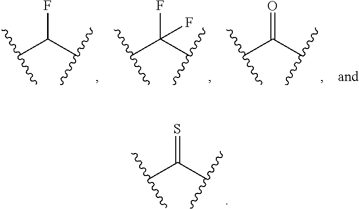

Non-limiting examples of optionally substituted —S— groups include:

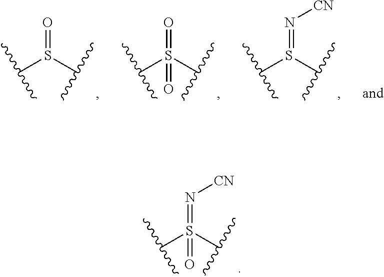

### Embodiments of “Alkyl”

In certain embodiments “alkyl” is a C₁-C₁₀alkyl, C₁-C₉alkyl, C₁-C₈alkyl, C₁-C₇alkyl, C₁-C₆alkyl, C₁-C₅alkyl, C₁-C₄alkyl, C₁-C₃alkyl, or C₁-C₂alkyl.

In certain embodiments “alkyl” has one carbon.

In certain embodiments “alkyl” has two carbons.

In certain embodiments “alkyl” has three carbons.

In certain embodiments “alkyl” has four carbons.

In certain embodiments “alkyl” has five carbons.

In certain embodiments “alkyl” has six carbons.

Non-limiting examples of “alkyl” include: methyl, ethyl, propyl, butyl, pentyl, and hexyl.

Additional non-limiting examples of “alkyl” include: isopropyl, isobutyl, isopentyl, and isohexyl.

Additional non-limiting examples of “alkyl” include: sec-butyl, sec-pentyl, and sec-hexyl.

Additional non-limiting examples of “alkyl” include: tert-butyl, tert-pentyl, and tert-hexyl.

Additional non-limiting examples of “alkyl” include: neopentyl, 3-pentyl, and active pentyl.

In an alternative embodiment the “alkyl” group is optionally substituted.

In an alternative embodiment the “alkenyl” group is optionally substituted.

In an alternative embodiment the “alkynyl” group is optionally substituted.

### Embodiments of “Haloalkyl”

In certain embodiments “haloalkyl” is a C₁-C₁₀haloalkyl, C₁-C₉haloalkyl, C₁-C₈haloalkyl, C₁-C₇haloalkyl, C₁-C₆haloalkyl, C₁-C₈haloalkyl, C₁-C₄haloalkyl, C₁-C₃haloalkyl, and C₁-C₂haloalkyl.

In certain embodiments “haloalkyl” has one carbon.

In certain embodiments “haloalkyl” has one carbon and one halogen.

In certain embodiments “haloalkyl” has one carbon and two halogens.

In certain embodiments “haloalkyl” has one carbon and three halogens.

In certain embodiments “haloalkyl” has two carbons.

In certain embodiments “haloalkyl” has three carbons.

In certain embodiments “haloalkyl” has four carbons.

In certain embodiments “haloalkyl” has five carbons.

In certain embodiments “haloalkyl” has six carbons.

Non-limiting examples of “haloalkyl” include:

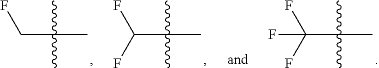

Additional non-limiting examples of “haloalkyl” include:

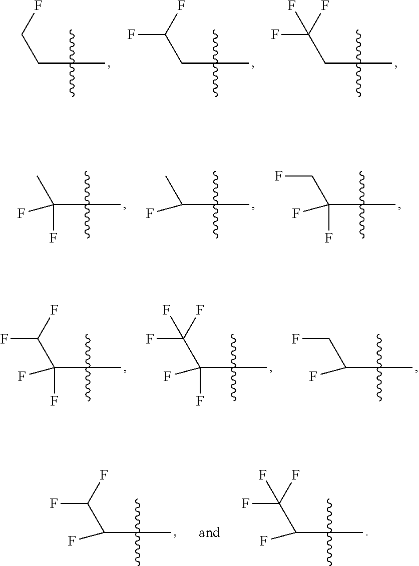

Additional non-limiting examples of “haloalkyl” include:

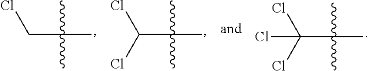

Additional non-limiting examples of “haloalkyl” include:

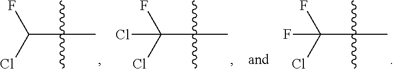

### Embodiments of “Heteroaryl”

Non-limiting examples of 5 membered “heteroaryl” groups include pyrrole, furan, thiophene, pyrazole, imidazole, triazole, isoxazole, oxazole, oxadiazole, oxatriazole, isothiazole, thiazole, thiadiazole, and thiatriazole.

Additional non-limiting examples of 5 membered “heteroaryl” groups include:

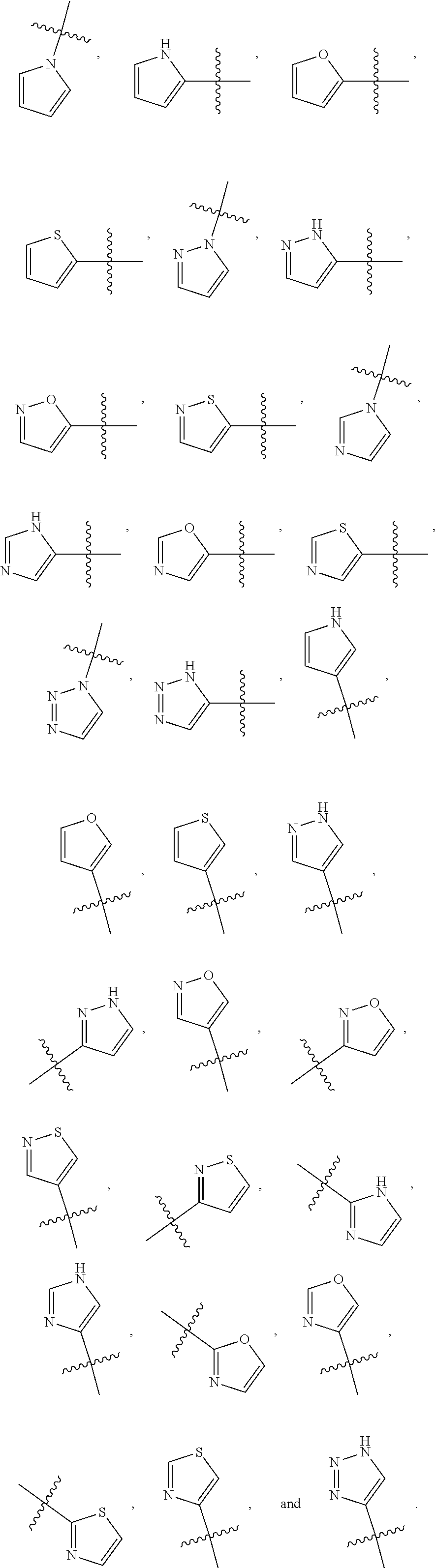

In certain embodiments “heteroaryl” is a 6 membered aromatic group containing 1, 2, or 3 nitrogen atoms (i.e. pyridinyl, pyridazinyl, triazinyl, pyrimidinyl, and pyrazinyl).

Non-limiting examples of 6 membered “heteroaryl” groups with 1 or 2 nitrogen atoms include:

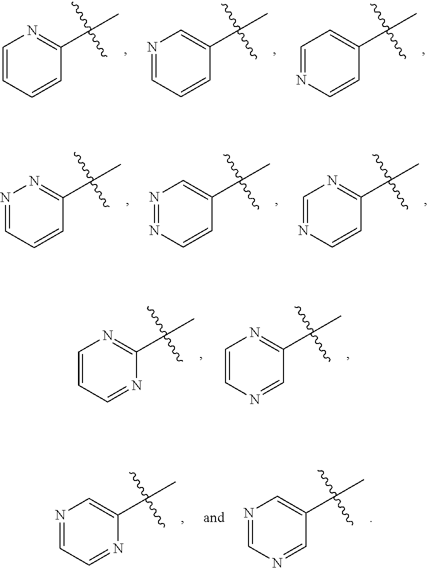

In certain embodiments “heteroaryl” is a 9 membered bicyclic aromatic group containing 1 or 2 atoms selected from nitrogen, oxygen, and sulfur.

Non-limiting examples of “heteroaryl” groups that are bicyclic include indole, benzofuran, isoindole, indazole, benzimidazole, azaindole, azaindazole, purine, isobenzofuran, benzothiophene, benzoisoxazole, benzoisothiazole, benzooxazole, and benzothiazole.

Additional non-limiting examples of “heteroaryl” groups that are bicyclic include:

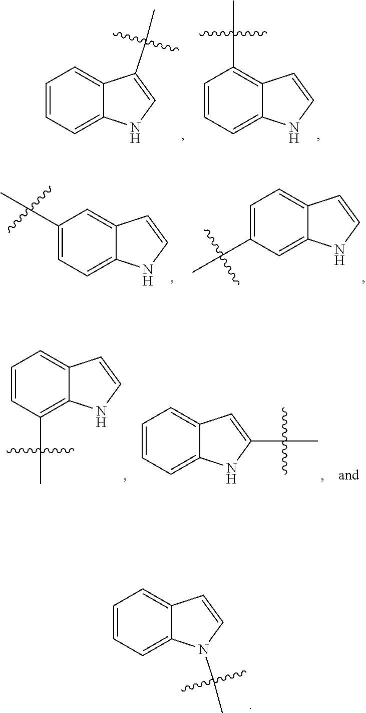

Additional non-limiting examples of “heteroaryl” groups that are bicyclic include:

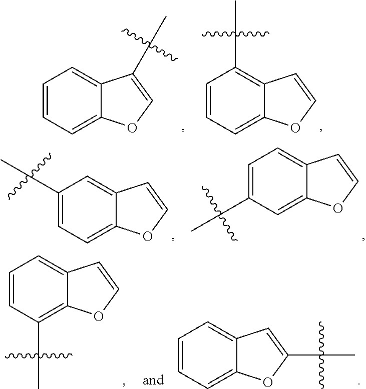

Additional non-limiting examples of “heteroaryl” groups that are bicyclic include:

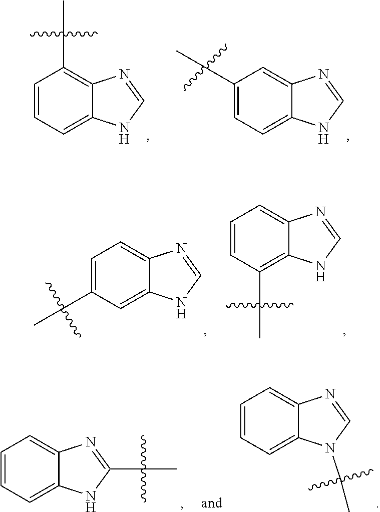

In certain embodiments “heteroaryl” is azaindole or benzimidazole.

In certain embodiments “heteroaryl” is a 10 membered bicyclic aromatic group containing 1 or 2 atoms selected from nitrogen, oxygen, and sulfur.

Non-limiting examples of “heteroaryl” groups that are bicyclic include quinoline, isoquinoline, quinoxaline, phthalazine, quinazoline, cinnoline, and naphthyridine.

Additional non-limiting examples of “heteroaryl” groups that are bicyclic include:

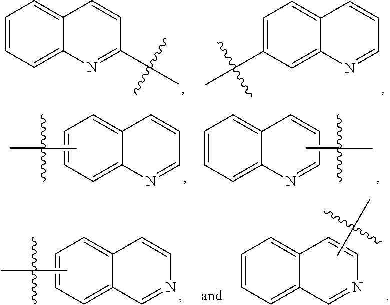

### Embodiments of “Heterocycle”

In certain embodiments “heterocycle” refers to a cyclic ring with one nitrogen and 3, 4, 5, 6, 7, or 8 carbon atoms.

In certain embodiments “heterocycle” refers to a cyclic ring with one nitrogen and one oxygen and 3, 4, 5, 6, 7, or 8 carbon atoms.

In certain embodiments “heterocycle” refers to a cyclic ring with two nitrogens and 3, 4, 5, 6, 7, or 8 carbon atoms.

In certain embodiments “heterocycle” refers to a cyclic ring with one oxygen and 3, 4, 5, 6, 7, or 8 carbon atoms.

In certain embodiments “heterocycle” refers to a cyclic ring with one sulfur and 3, 4, 5, 6, 7, or 8 carbon atoms.

Non-limiting examples of “heterocycle” include aziridine, oxirane, thiirane, azetidine, 1,3-diazetidine, oxetane, and thietane.

Additional non-limiting examples of “heterocycle” include pyrrolidine, 3-pyrroline, 2-pyrroline, pyrazolidine, and imidazolidine.

Additional non-limiting examples of “heterocycle” include tetrahydrofuran, 1,3-dioxolane, tetrahydrothiophene, 1,2-oxathiolane, and 1,3-oxathiolane.

Additional non-limiting examples of “heterocycle” include piperidine, piperazine, tetrahydropyran, 1,4-dioxane, thiane, 1,3-dithiane, 1,4-dithiane, morpholine, and thiomorpholine.

Additional non-limiting examples of “heterocycle” include indoline, tetrahydroquinoline, tetrahydroisoquinoline, and dihydrobenzofuran wherein the point of attachment for each group is on the heterocyclic ring.

Bicyclic and tricyclic substituents described herein are attached through the named functional group. For example, if an R substituent is a bicyclic heterocycle the attachment point is on a heterocyclic ring. For example,

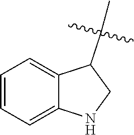 [SMILES: *C1CNc2ccccc21]

is a “heterocycle” group. However,

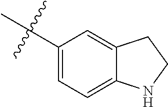 [SMILES: *c1ccc2c(c1)CCN2]

is an “aryl” group.

Non-limiting examples of “heterocycle” also include:

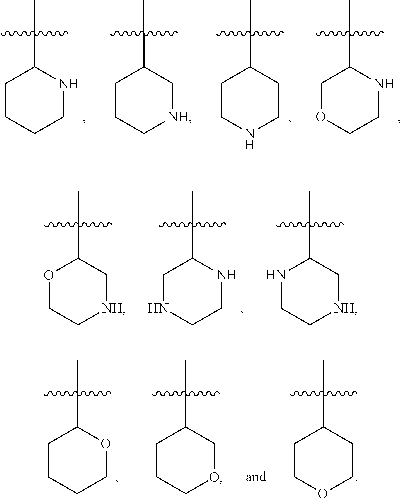

Additional non-limiting examples of“heterocycle” include:

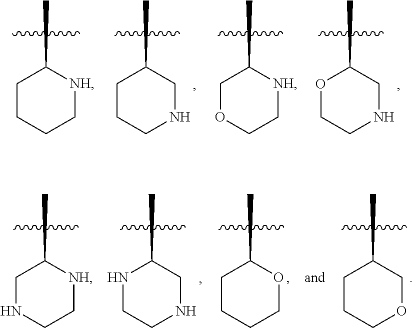

Additional examples of “heterocycle” include:

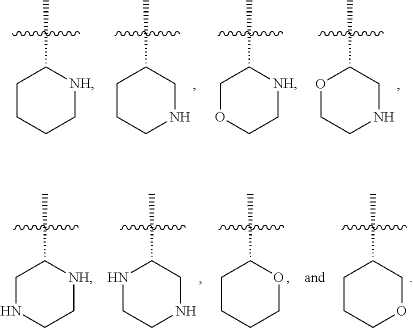

Non-limiting examples of “heterocycle” also include:

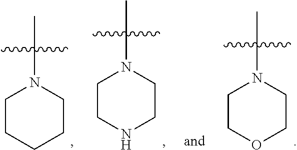

Non-limiting examples of “heterocycle” also include:

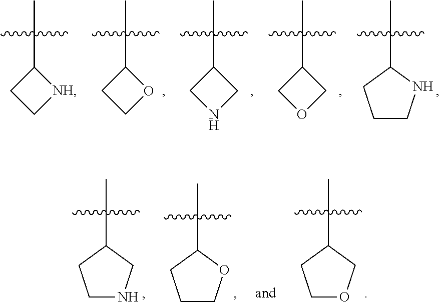

Additional non-limiting examples of “heterocycle” include:

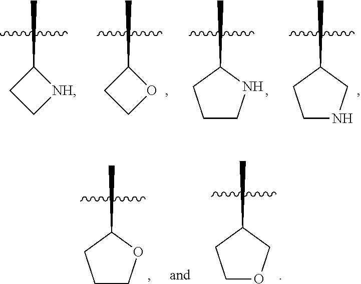

Additional non-limiting examples of “heterocycle” include:

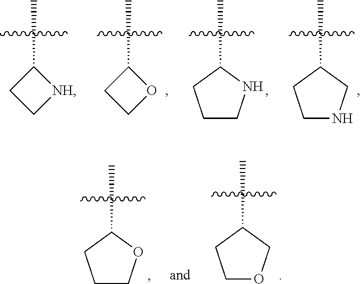

### Aryl

In certain embodiments “aryl” is a 6 carbon aromatic group (phenyl).

In certain embodiments “aryl” is a 10 carbon aromatic group (naphthyl).

In certain embodiments “aryl” is a 6 carbon aromatic group fused to a heterocycle wherein the point of attachment is the aryl ring. Non-limiting examples of “aryl” include indoline, tetrahydroquinoline, tetrahydroisoquinoline, and dihydrobenzofuran wherein the point of attachment for each group is on the aromatic ring.

For example

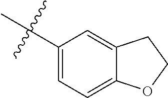 [SMILES: *c1ccc2c(c1)CCO2]

is an “aryl” group.

However,

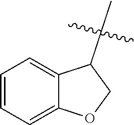 [SMILES: *C1COc2ccccc21]

is a “heterocycle” group.

### Embodiments of “Arylalkyl”

Non-limiting examples of “arylalkyl” include:

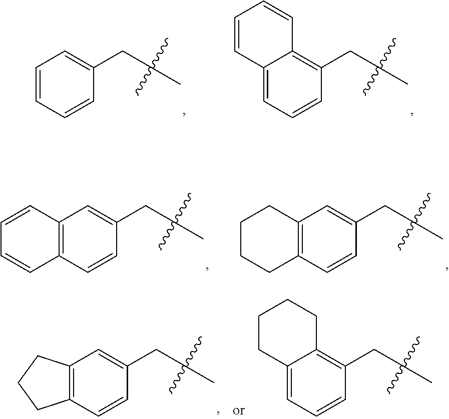

In certain embodiments “arylalkyl” is

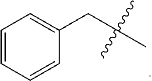 [SMILES: *Cc1ccccc1]

In certain embodiments the “arylalkyl” refers to a 2 carbon alkyl group substituted with an aryl group.

Non-limiting examples of “arylalkyl” include:

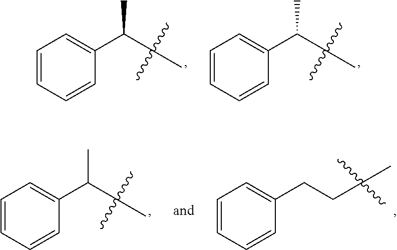

### II. Extracellular Protein Degradation

A wide range of well-known and characterized extracellular proteins can cause, modulate, or amplify diseases in vivo, such as abnormal cellular proliferation such as tumors and cancer, autoimmune disorders, inflammation and aging-related diseases. For example, extracellular proteins such as growth factors, cytokines, and chemokines bind to cell surface receptors, often initiate aberrant signaling in multiple diseases such as cancer and inflammation.

An extracellular protein degrader described herein or its pharmaceutically acceptable salt and/or its pharmaceutically acceptable compositions can be used to treat a disorder which is mediated by the Target Extracellular Protein that binds to the Extracellular Protein Targeting Ligand. The described degraders are capable of targeting specific Extracellular Proteins that mediate pathological disorders for lysosomal degradation. The Target Extracellular Protein may modulate a disorder in a human via a mechanism of action such as modification of a biological pathway, pathogenic signaling, or modulation of a signal cascade or cellular entry. In certain embodiments, the Target Extracellular Protein is a protein that is not druggable in the classic sense in that it does not have a binding pocket or an active site that can be inhibited or otherwise bound, and cannot be easily allosterically controlled. In another embodiment, the Target Extracellular Protein is a protein that is druggable in the classic sense, yet for therapeutic purposes, degradation of the protein is preferred to inhibition. The Target Extracellular Protein is recruited with an Extracellular Protein Targeting Ligand, which is a ligand for the Target Extracellular Protein. Typically, the Extracellular Protein Targeting Ligand binds the Target Extracellular Protein in a non-covalent fashion. In an alternative embodiment, the Target Extracellular Protein is covalently bound to the Extracellular Protein Targeting Ligand in a covalent manner that can be irreversible or reversible.

Accordingly, in some embodiments, a method to treat a host with a disorder mediated by a Target Extracellular Protein is provided that includes administering an effective amount of a degrader targeting the Target Extracellular Protein to the host, typically a human, optionally in a pharmaceutically acceptable composition.

The Target Extracellular Protein can be any amino acid sequence to which the degrader comprising an Extracellular Protein Targeting Ligand can be bound which through degradation thereof, results in a beneficial therapeutic effect. In certain embodiments, the Target Extracellular Protein is a non-endogenous peptide such as that from a pathogen or toxin. In another embodiment, the Target Extracellular Protein can be an endogenous protein that mediates a disorder. The endogenous protein can be either the normal form of the protein or an aberrant form. For example, the Target Extracellular Protein can be an extracellular mutant protein, or a protein, for example, where a partial, or full, gain-of-function or loss-of-function is encoded by nucleotide polymorphisms. In some embodiments, the degrader targets the aberrant form of the protein and not the normal form of the protein.

The Extracellular Protein Targeting Ligand is a ligand which covalently or non-covalently binds to a Target Extracellular Protein which has been selected for lysosomal degradation. In certain embodiments the Extracellular Protein Targeting Ligand is a small molecule or moiety (for example a peptide, nucleotide, antibody fragment, aptamer, biomolecule, or other chemical structure) that binds to a Target Extracellular Protein, and wherein the Target Extracellular Protein is a mediator of disease in a host as described in detail below. Exemplary Extracellular Protein Targeting Ligands are provided in the Figures.

### Anchor Bond

The Extracellular Protein Targeting Ligand (“EPTL”) is covalently bound to Linker in the ASGPR-binding extracellular protein degrader compound through the Anchor Bond (which is the chemical bond between the EPTL and either Linker B, Linker C or Linker D). This bond can be placed at any location on the ligand that does not unacceptably disrupt the ability of the EPTL to bind to the Target Extracellular Protein. The Anchor Bond is depicted on the nonlimiting examples of Extracellular Protein Target Ligands in the figures as:

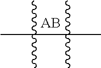 [SMILES: **]

A number of exemplary Target Extracellular Proteins for medical therapy described below have characterizing structural information in the well-known Protein Data Bank (“PDB”), which is a database for the three-dimensional structural information for large biological molecules such as proteins and nucleic acids. PDB includes x-ray crystallography and other information submitted by scientists around the world, and is freely accessible. See for example www.rcsb.org; www.wwpdb.org and www.uniprot.org. Using the PDB codes for example provided in Section ** or in the Data Bank itself, and technical references provided herein or otherwise publicly available, the skilled artisan can determine appropriate locations where the EPTL can be linked through an Anchor Bond to Linker B, Linker C or Linker D to the ASGPR-binding moiety. For many of these proteins, published references describe how a range of ligands bind to the Target Extracellular Proteins, and from this information, one can determine reasonable Anchor Bond locations.

For example, the skilled artisan can use available visualization tools, including those available on the PDB website, to determine where the Extracellular Protein Targeting Ligand docks into to the Target Extracellular Protein. The skilled artisan can also import the crystal structure and the selected Extracellular Protein Targeting Ligand of interest into modeling software (including for example PyMOL, Glide, Maestro, RasMol, Visual Molecular Dynamics, Jmol, and AutoDock) to determine what portion of the Extracellular Protein Targeting Ligand is bound to the Target Extracellular Protein. The ASGPR ligand is then bound through the Linker and the Anchor Bond at a point that does not unduly adversely affect binding to the Target Extracellular Protein.

### Optional Substituents

In certain embodiments an Extracellular Protein Targeting Ligand described herein, for example in one of the figures, is optionally substituted with 1, 2, 3, or 4 optional substituents independently selected from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, cyano, nitro, C(O)R³,

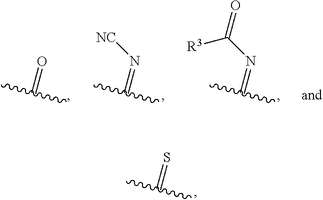

wherein the optional substituent is selected such that a stable compound results.

In certain embodiments the Target Extracellular Protein is selected from IgA, IgG, IgE, TNF-alpha, IL-1, IL-2, IL-6, IFN-γ, VEGF, TGF-β1, PCSK-9, CPB2, ChE, CCL2, Factor VII, Factor IX, CD40L, Factor Xa, Factor XI, Factor XIa, Factor XII, Factor XIII, FGF1, FGF2, FN1, IL-5, IL-8, IL-10, IL-21, IL-22, Kallikrein 1, LPL, MMP1, MIF, GIF, L-dopachrome isomerase, or phenylpyruvate tautomerase, neutrophil elastase, Prothrombin, KLKB1, PLG, PAI-1, endothelial plasminogen activator inhibitor, serpin E1, phospholipases A2, PLA2, PA21B, PLA2G1B, PLA2-IB, PLA2, PLA2A, PA2IIA, PLA2G2A, PLA2-IIA, PGF, plasminogen activator, tissue type (tPA, PLAT), Transforming growth factor beta 2 (TGF-β2, TGFB2), thrombospondin 1, Urokinase, Urokinase-type plasminogen activator, complement factor B, complement factor D, target complement factor H, and complement component 5.

In certain embodiments, where the Target Extracellular Protein has a receptor the Target Extracellular Protein can be used to degrade the receptor.

In certain embodiments the Extracellular Protein Targeting Ligand is a ligand for a protein selected from IgA, IgG, IgE, TNF-alpha, IL-1, IL-2, IL-6, IFN-γ, VEGF, TGF-β1, PCSK-9, CPB2, ChE, CCL2, Factor VII, Factor IX, CD40L, Factor Xa, Factor XI, Factor XIa, Factor XII, Factor XIII, FGF1, FGF2, FN1, IL-5, IL-8, IL-10, IL-21, IL-22, Kallikrein 1, LPL, MMP1, MIF, GIF, L-dopachrome isomerase, or phenylpyruvate tautomerase, neutrophil elastase, Prothrombin, KLKB1, PLG, PAI-1, endothelial plasminogen activator inhibitor, serpin E1, phospholipases A2, PLA2, PA21B, PLA2G1B, PLA2-IB, PLA2, PLA2A, PA2IIA, PLA2G2A, PLA2-JIA, PGF, plasminogen activator, tissue type (tPA, PLAT), Transforming growth factor beta 2 (TGF-β2, TGFB2), thrombospondin 1, Urokinase, Urokinase-type plasminogen activator, complement factor B, complement factor D, target complement factor H, and complement component 5.

In other embodiments the Extracellular Protein Targeting Ligand is a ligand for anti-B1AR.

### Amino Acids

In certain embodiments the Extracellular Protein Targeting Ligand comprises one or more amino acids. The invention contemplates using natural amino acids, unnatural amino acids, or any combination thereof to achieve desired targeting ligand properties.

The term “natural amino acid” refers to an amino acid selected from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine.

In certain embodiments a natural amino acid is replaced with a corresponding unnatural amino acid for example substituting a phenylalanine for a 4-chloro-phenylalanine. Non-limiting examples of unnatural amino acids include: 4-chloro-phenylalanine, 3-fluoro-phenylalanine, 4-trifluoromethyl-phenylalanine, 3,4-dichloro-phenylalanine, 4-phenyl-phenylalanine, N-methylalanine, N-methylglutamic acid, N-methylphenylalanine, and homoserine.

Additional examples of non-natural amino acids include:

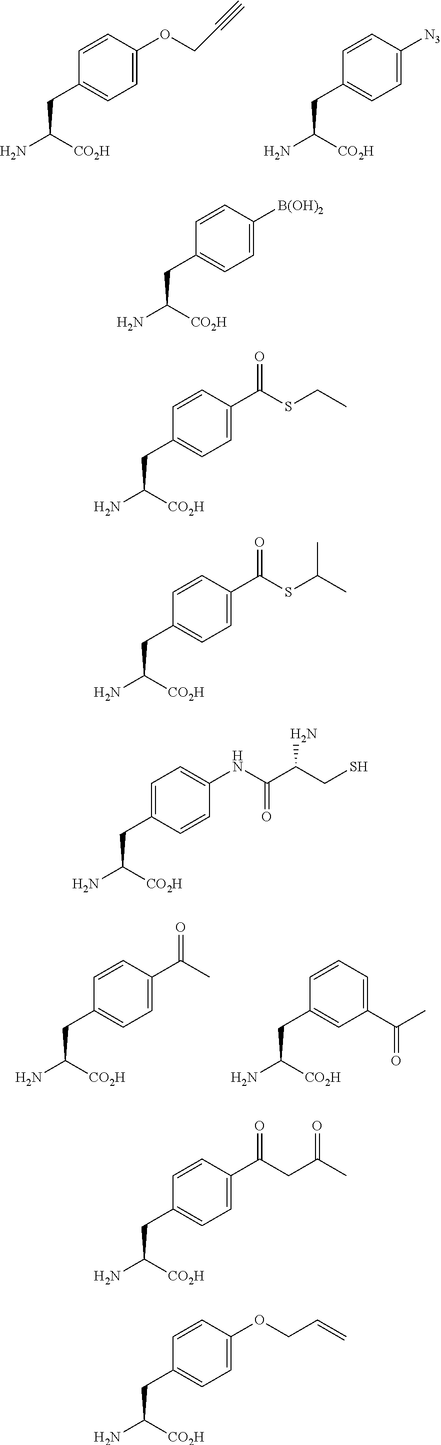

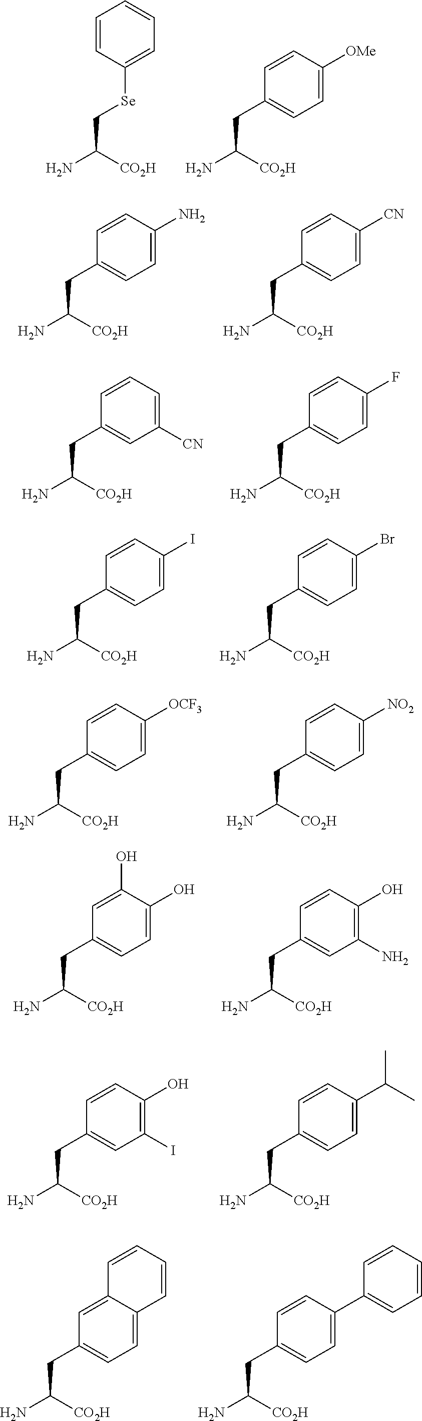

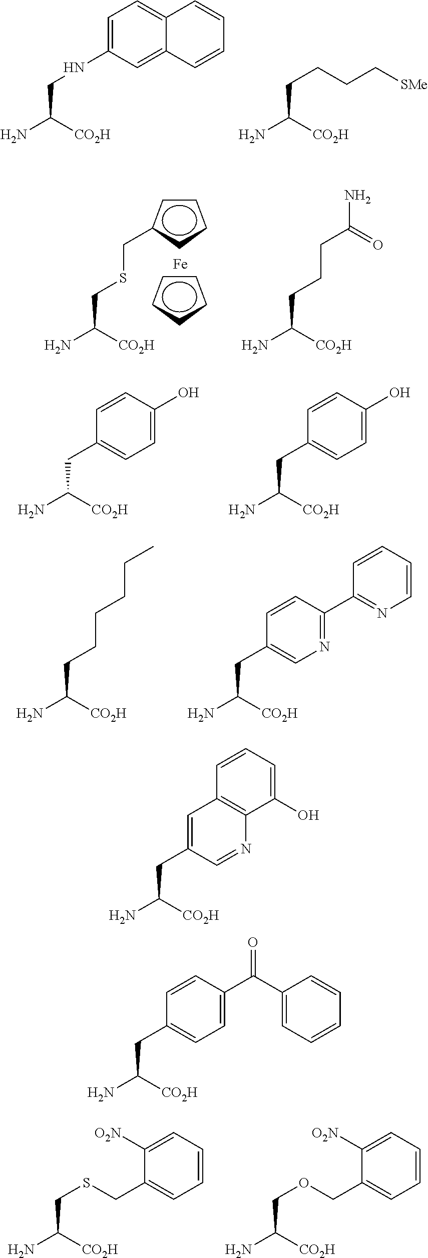

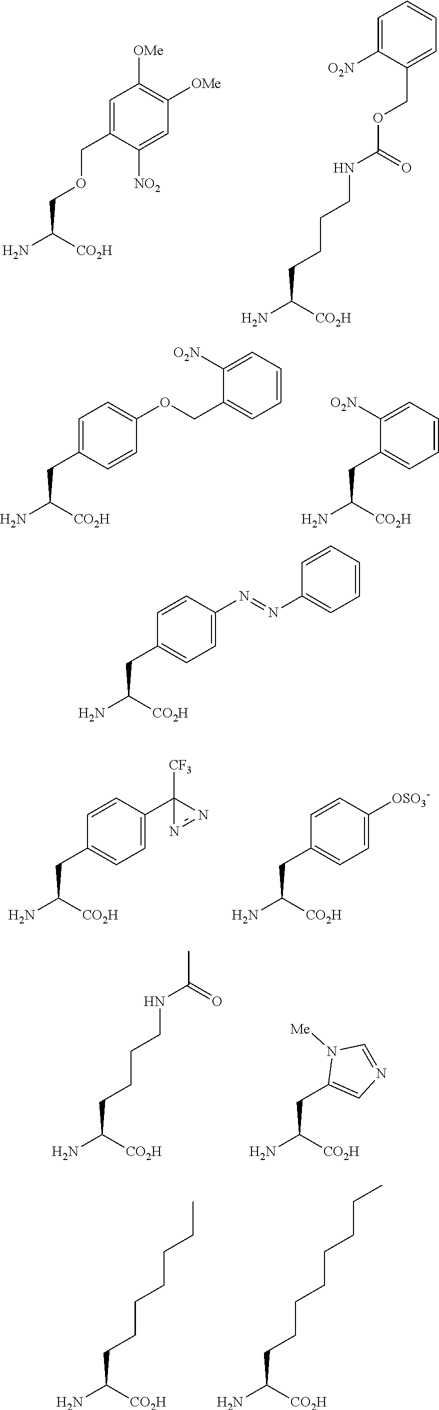

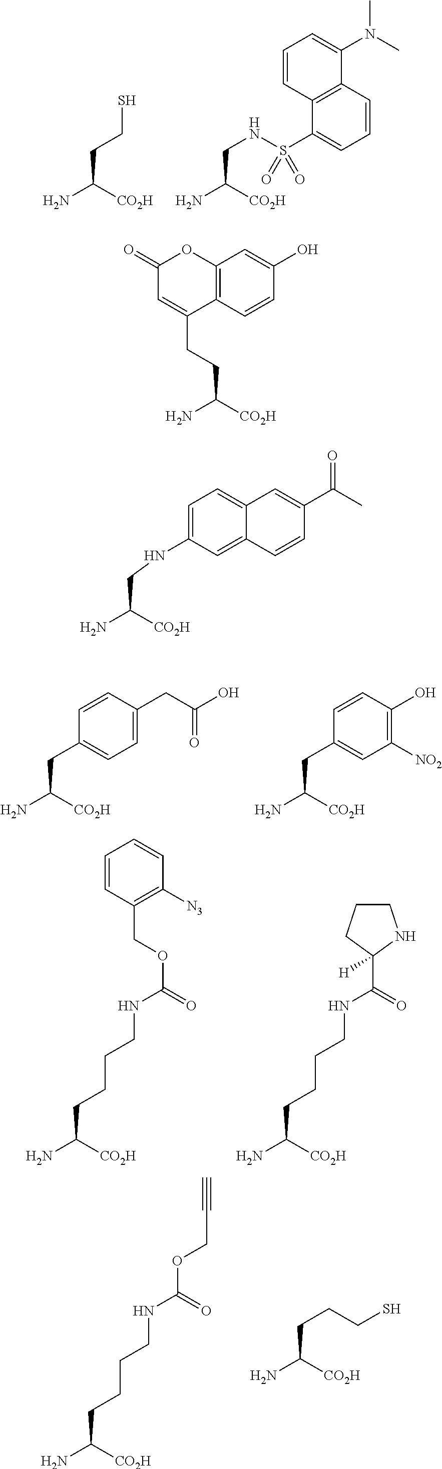

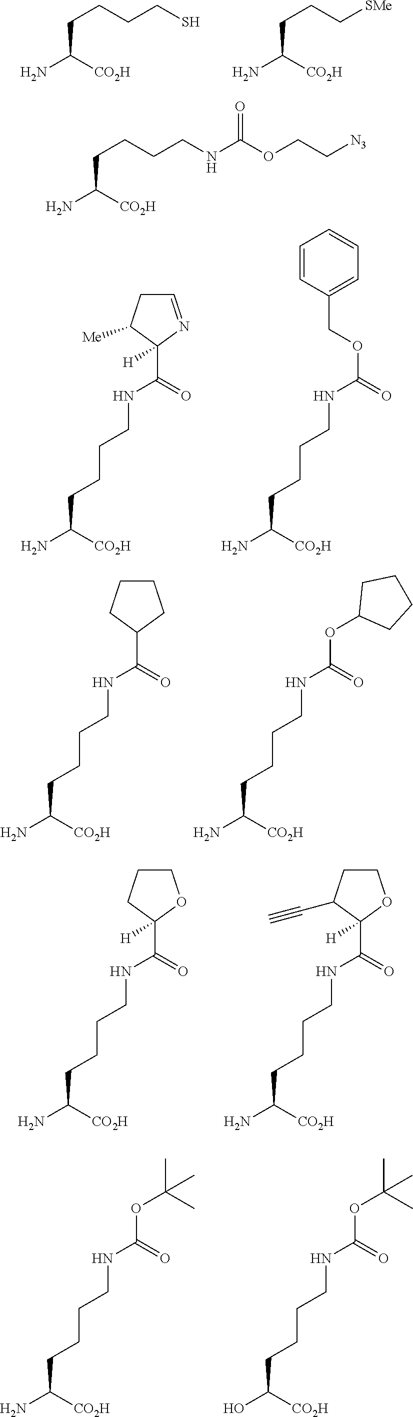

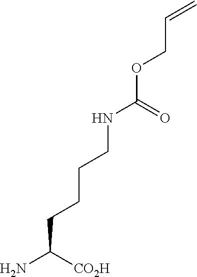 [SMILES: C=CCOC(=O)NCCCC[C@H](N)C(=O)O]

In certain embodiments the Extracellular Protein Targeting Ligand is a sequence of amino acids. In certain embodiments the amino acid sequence is connected to the Linker portion of the molecule by a bond to a terminal amine. In certain embodiments the amino acid sequence is connected to the Linker portion of the molecule by a bond to a terminal carboxylic acid (e.g. an ester or amide). In certain embodiments the peptide includes an amine, hydroxyl, or carboxylic acid side chain and the linker may be bound to one of these sidechains.

For example, when the amino acid sequence is SEQ ID NO. 1 MLKKIE non-limiting examples of locations wherein the peptide may be attached to the linker include.

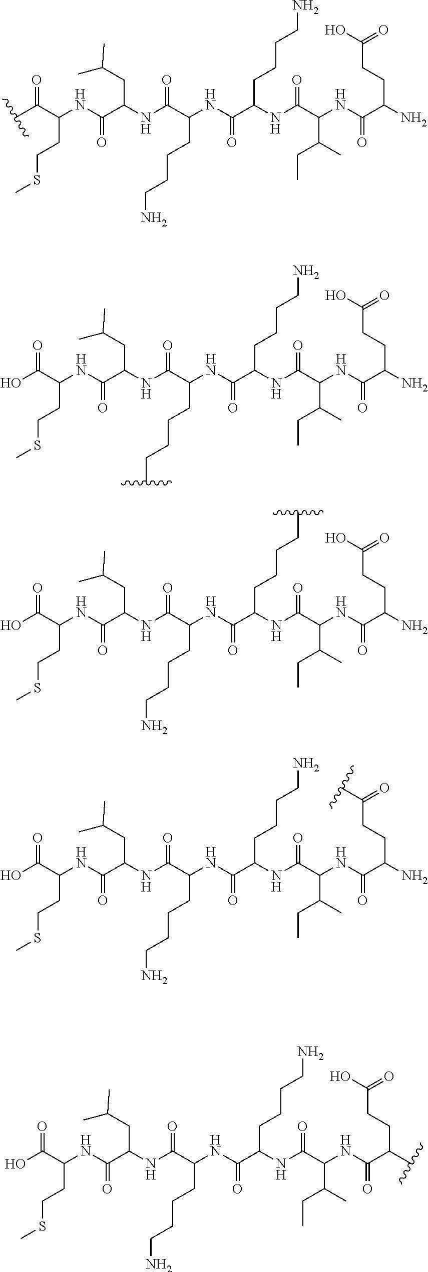 [SMILES: *C(=O)C(CCSC)NC(=O)C(CC(C)C)NC(=O)C(CCCCN)NC(=O)C(CCCCN)NC(=O)C(NC(=O)C(N)CCC(=O)O)C(C)CC ; *CCCCC(NC(=O)C(CCCCN)NC(=O)C(NC(=O)C(N)CCC(=O)O)C(C)CC)C(=O)NC(CC(C)C)C(=O)NC(CCSC)C(=O)O ; *CCCCC(NC(=O)C(NC(=O)C(N)CCC(=O)O)C(C)CC)C(=O)NC(CCCCN)C(=O)NC(CC(C)C)C(=O)NC(CCSC)C(=O)O ; *C(=O)CCC(N)C(=O)NC(C(=O)NC(CCCCN)C(=O)NC(CCCCN)C(=O)NC(CC(C)C)C(=O)NC(CCSC)C(=O)O)C(C)CC ; *C(CCC(=O)O)C(=O)NC(C(=O)NC(CCCCN)C(=O)NC(CCCCN)C(=O)NC(CC(C)C)C(=O)NC(CCSC)C(=O)O)C(C)CC]

The amino acid sequence can be attached to the Linker with chemistry described herein and as otherwise known in the art. For example, when the desired linking group is an amide the linker can be presented with an amine, carboxylic acid, ester or other amide precursor and the targeting ligand can be attached with an amide coupling reaction such as a HATU or HBTU coupling reaction.

Non-limiting examples of Extracellular Protein Targeting Ligands that are a sequence of amino acids include aptamers, antibodies, and peptides. In certain embodiments the left most amino acid listed in the sequence listing is the C-terminus. In other embodiments the right most amino acid listed in the sequence listing is the C-terminus.

In certain embodiments the amino acid sequence refers to a sequence without specified chirality. In other embodiments the amino acid sequence is all D-, all L-, or a mixture of D- and L-amino acids.

When peptides are denoted by an amino acid sequence in a structure drawn herein the left side of the peptide is typically the N-terminus and the right side is typically the C-terminus unless excluded by context. For example, the proline in PIESESLK is attached through the nitrogen of the N-terminus to the linker in the structure below.

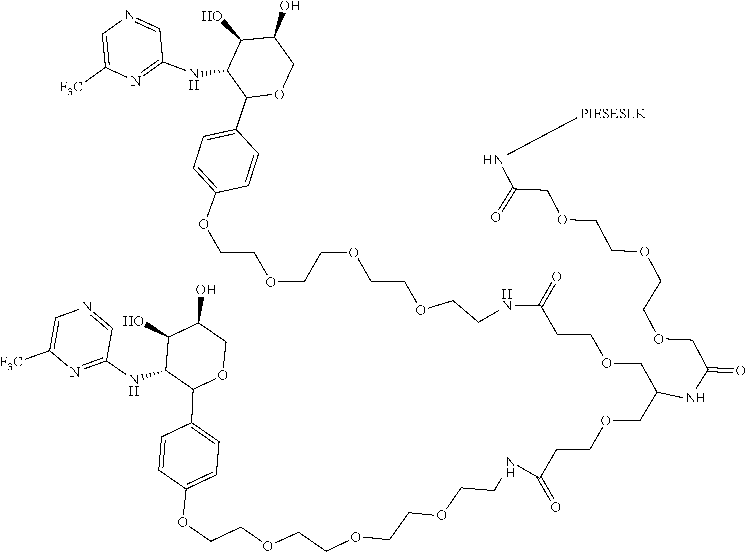

For clarity the NH that is part of the amide is part of proline and the CO is part of the linker.

When the lysine in PIESESLK is attached through the carbonyl of the C-terminus to the linker in the structure below.

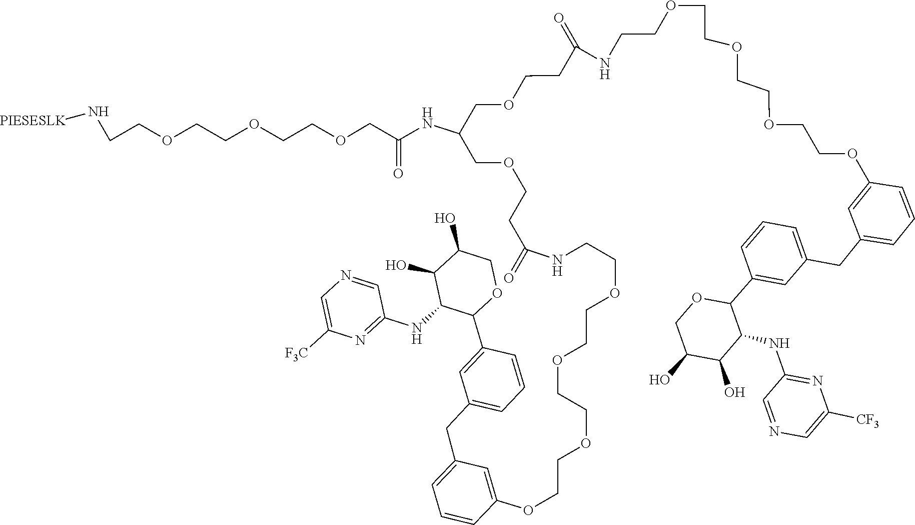

For clarity the NH that is bound to the lysine is part of the linker and the lysine is bound to the NH by the carbonyl that is part of the C-terminus.

### IL-1

In some embodiments, the Target Extracellular Protein is human interleukin-1 (IL-1) (UniProtKB—P01584 (IL1B_HUMAN)). IL-1 is a potent proinflammatory cytokine. Initially discovered as the major endogenous pyrogen, induces prostaglandin synthesis, neutrophil influx and activation, T-cell activation and cytokine production, B-cell activation and antibody production, and fibroblast proliferation and collagen production. IL-1 promotes Th17 differentiation of T-cells, and Synergizes with IL12/interleukin-12 to induce IFNG synthesis from T-helper 1 (Th1) cells. IL-1 has been implicated in a number of auto-inflammatory and autoimmune disorders, including, but not limited to, Blau syndrome, cryopyrin-associated periodic syndromes, familial Mediterranean fever, Majeed syndrome; mevalonate kinase deficiency syndrome, pyogenic arthritis-pyoderma gangrenosum-acne syndrome, tumor necrosis factor receptor-associated periodic syndrome, Behçet's Disease, Sjogren's Syndrome, gout and chondrocalcinosis, periodic fever, aphthous stomatitis, pharyngitis, and cervical adenitis (or PFAPA) syndrome, rheumatoid arthritis, Type 2 diabetes mellitus, acute pericarditis, Chronic interstitial lung diseases (ILDs), Still's Disease,

The Protein Data Bank website provides the crystal structure of IL-1 searchable by 9ILB (Yu, B., et al., Proc Natl Acad Sci USA, 1999, 96 103-108); II1B (Finzel, B. C., et al., J Mol Biol., 1989, 209 779-791); and 3040 (Wang et al., Nat. Immunol., 2010, 11: 905-911); as well as the crystal structure of IL-1 bound to various compounds searchable by 4G6J (Blech, M., et al., J Mol Biol., 2013, 425 94-111); 5BVP (Rondeau e al., MAbs, 2015, 7 1151-1160); and 3LTQ (Barthelmes, K., et al., J Am Chem. Soc., 2011, 133 808-819). Additionally, Guy et al., provides insight into the crystal structure of a small antagonist peptide bound to interleukin-1 receptor type 1 (Guy et al., The Journal of Biological Chemistry, 2000, 275, 36927-36933).

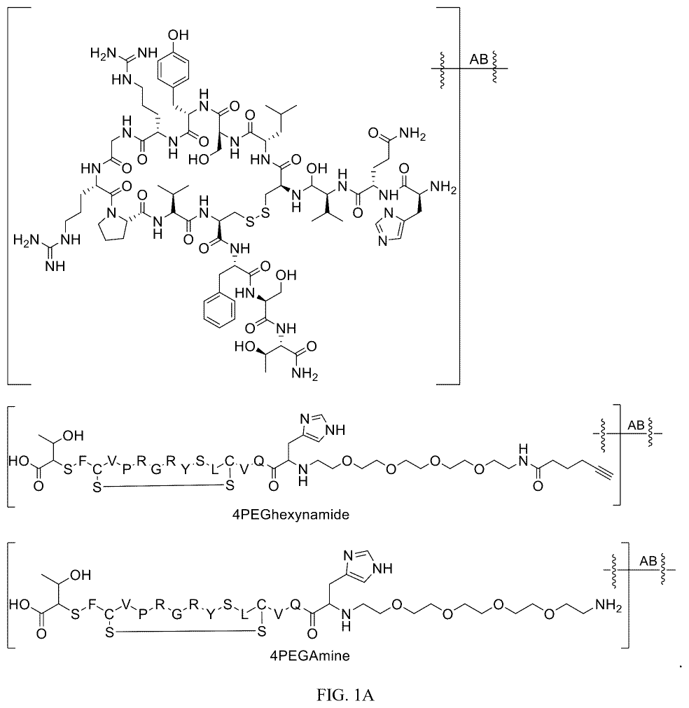

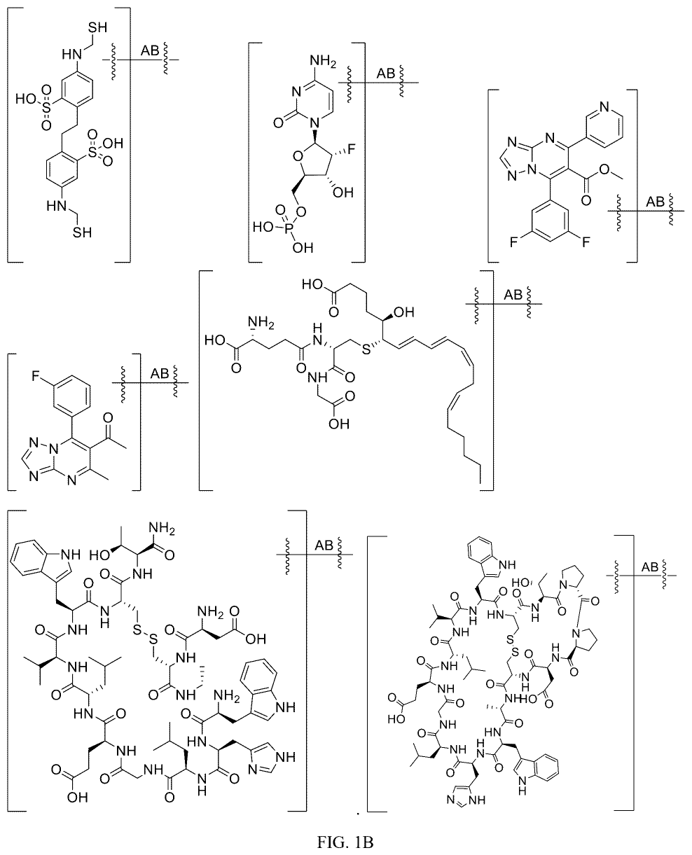

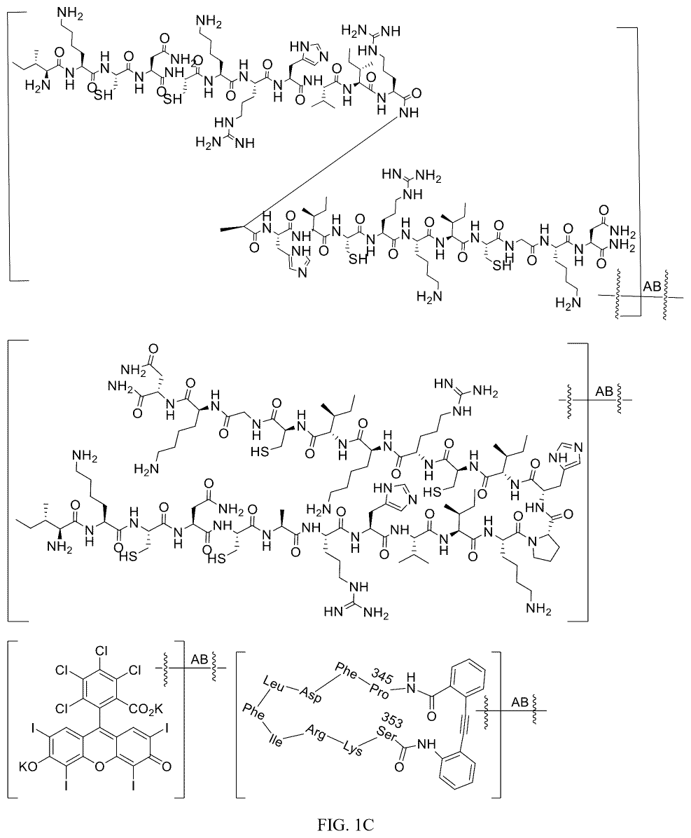

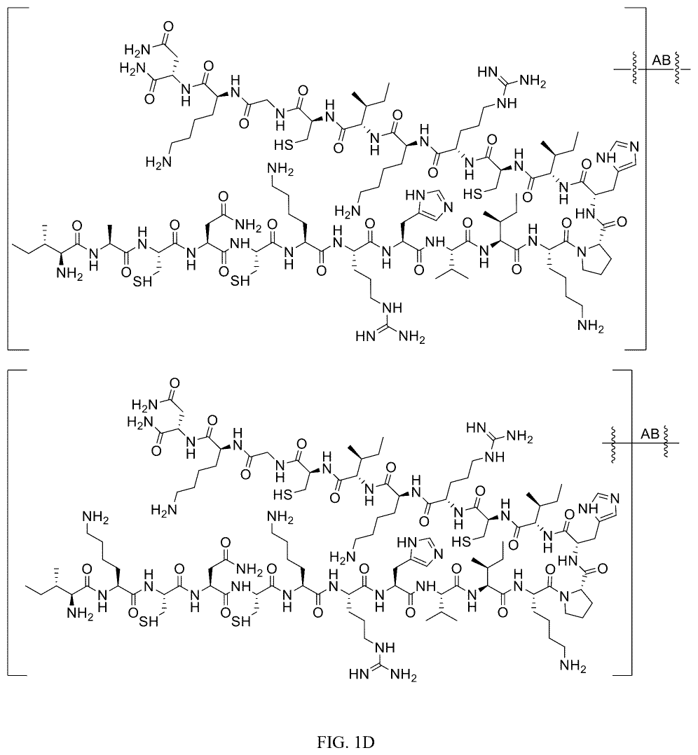

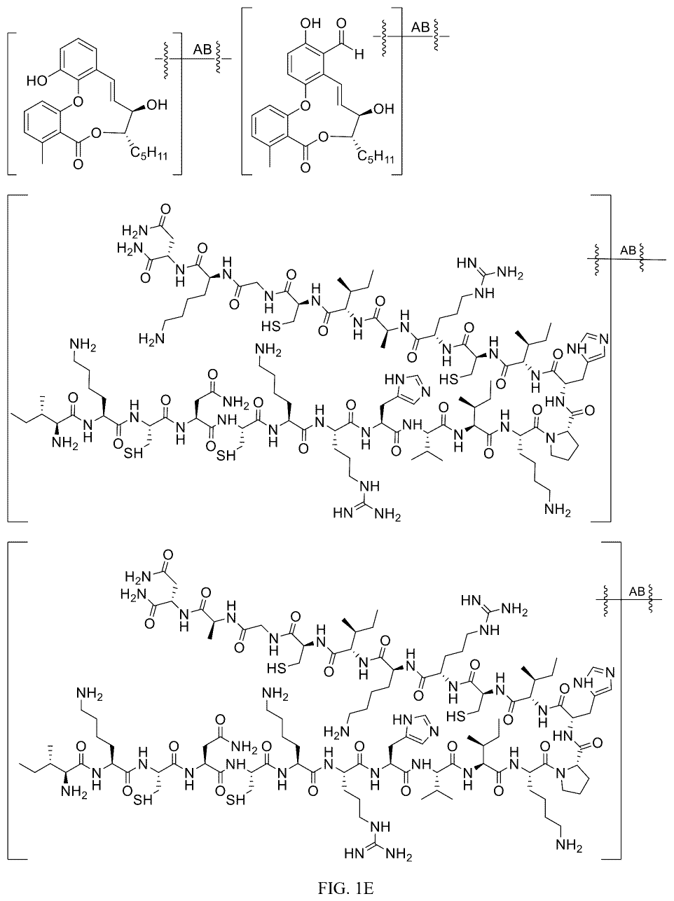

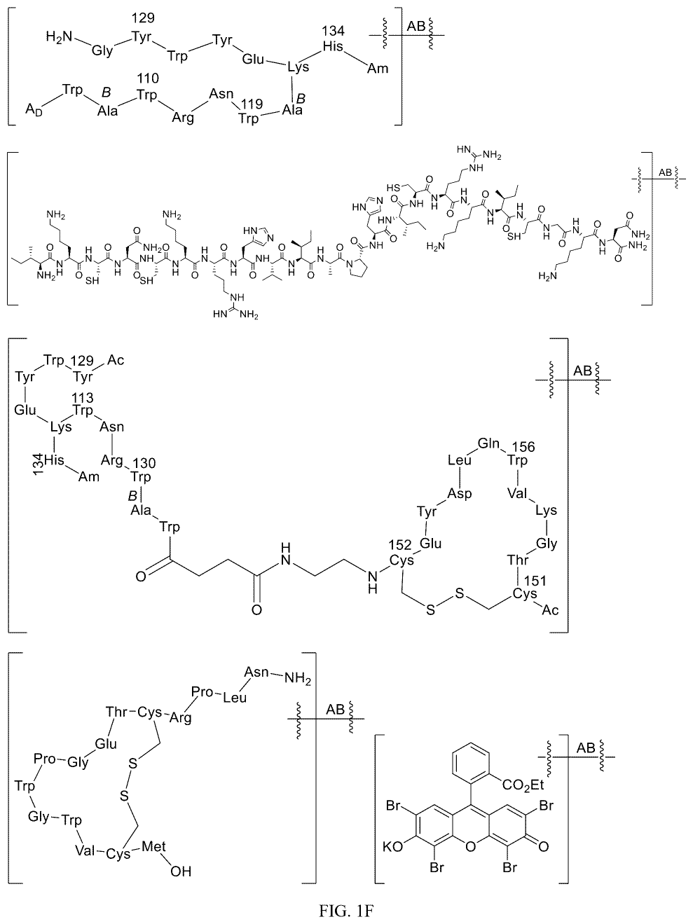

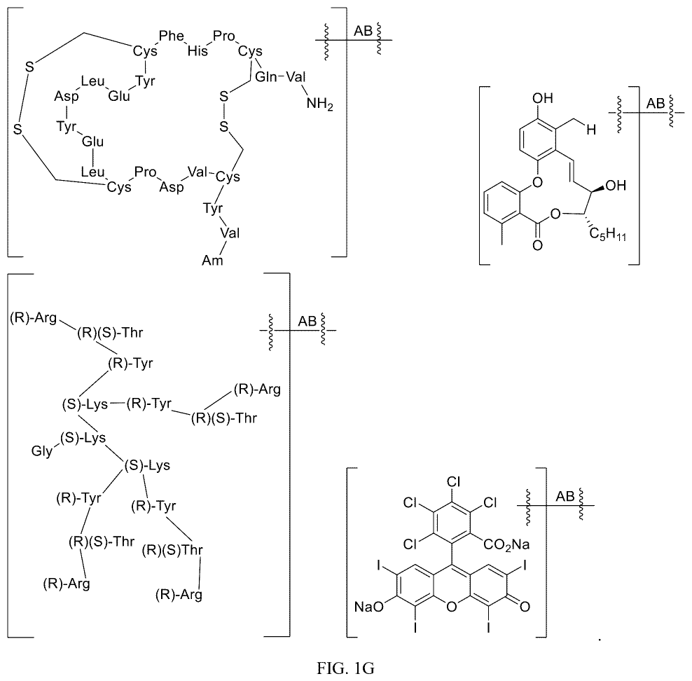

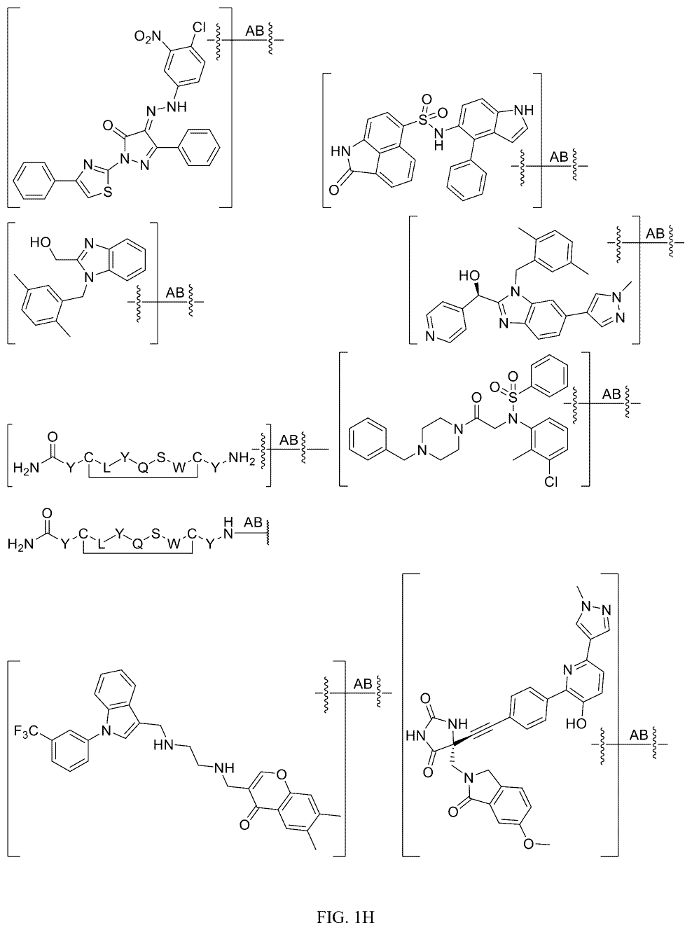

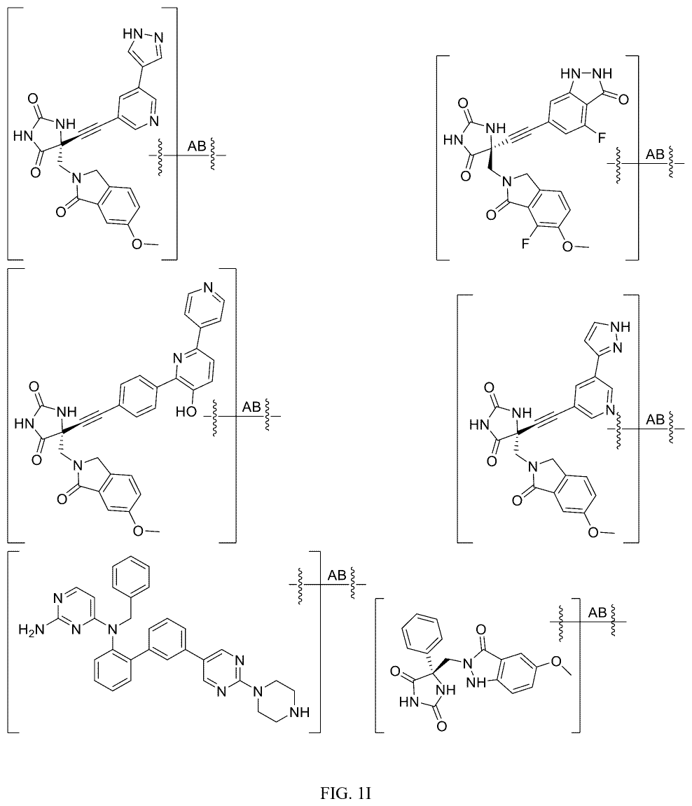

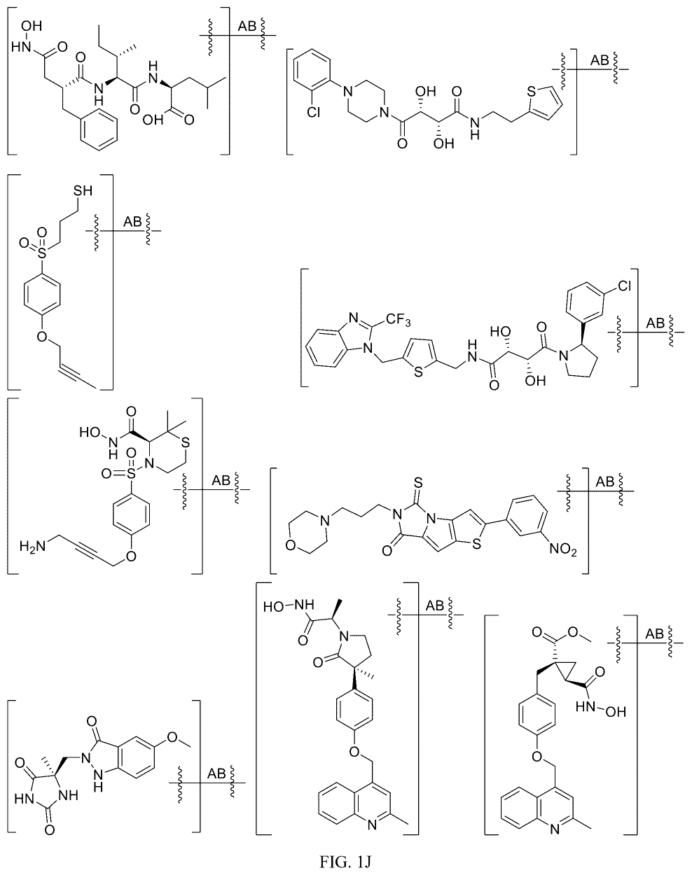

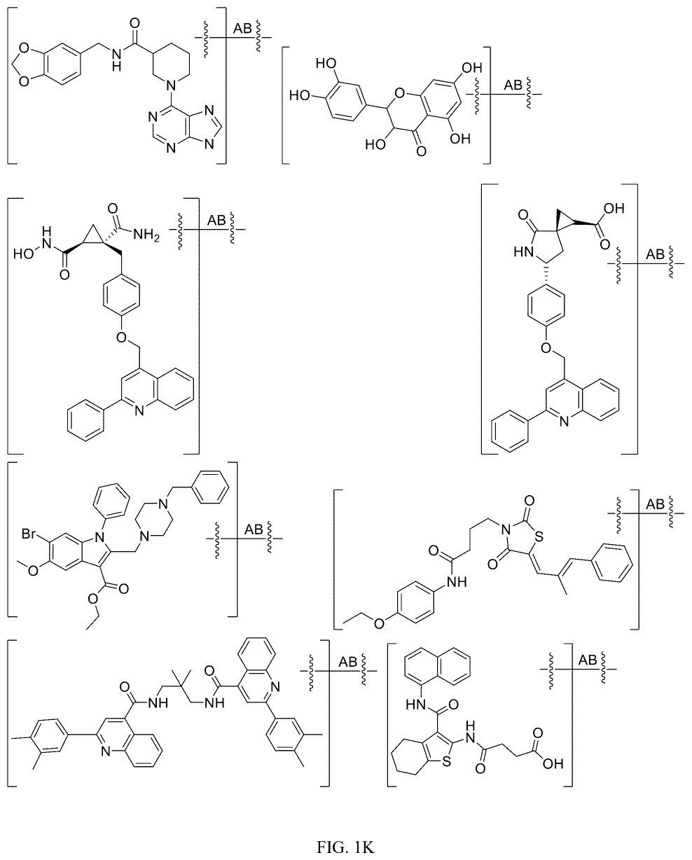

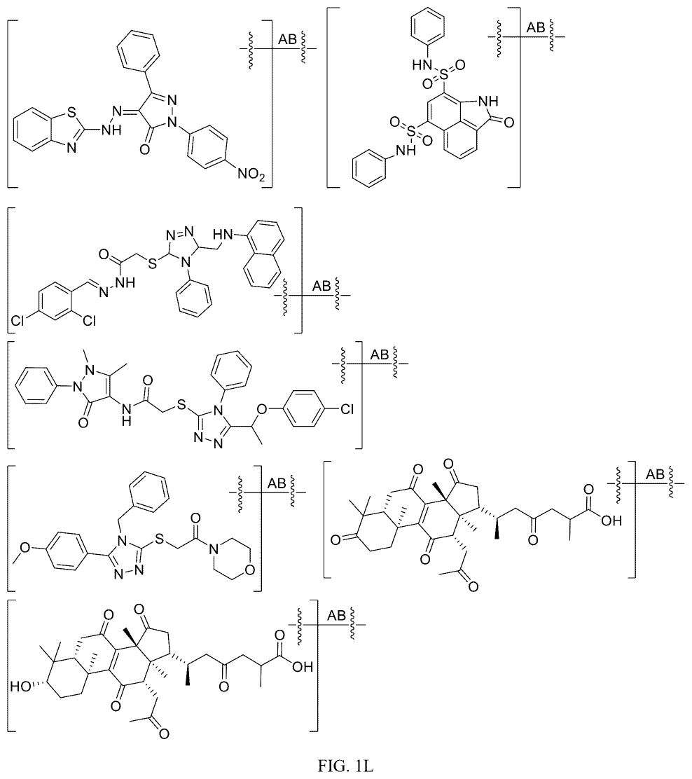

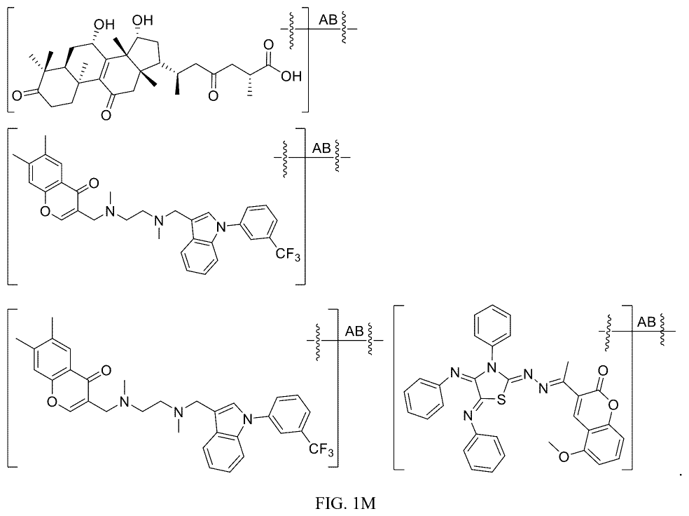

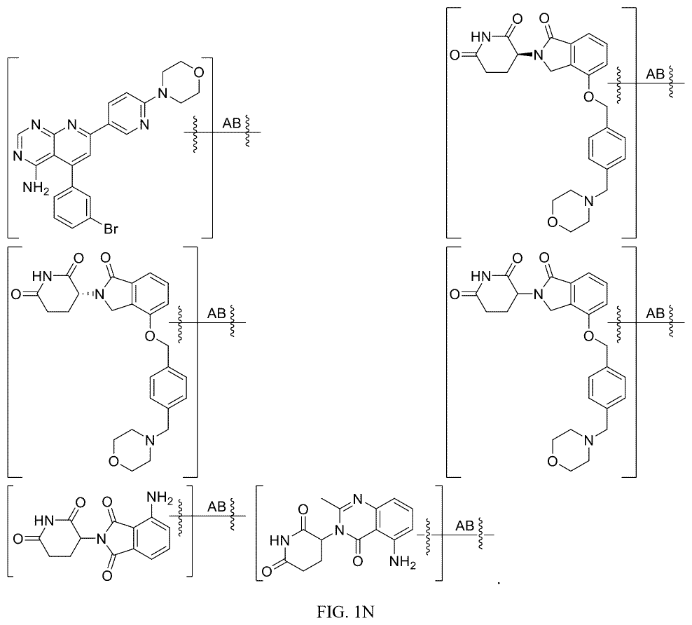

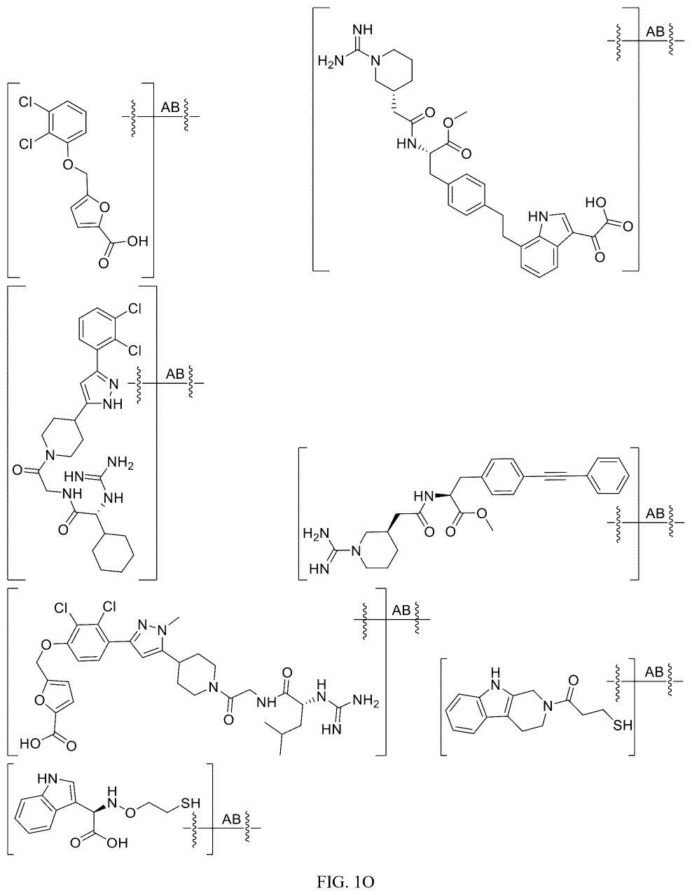

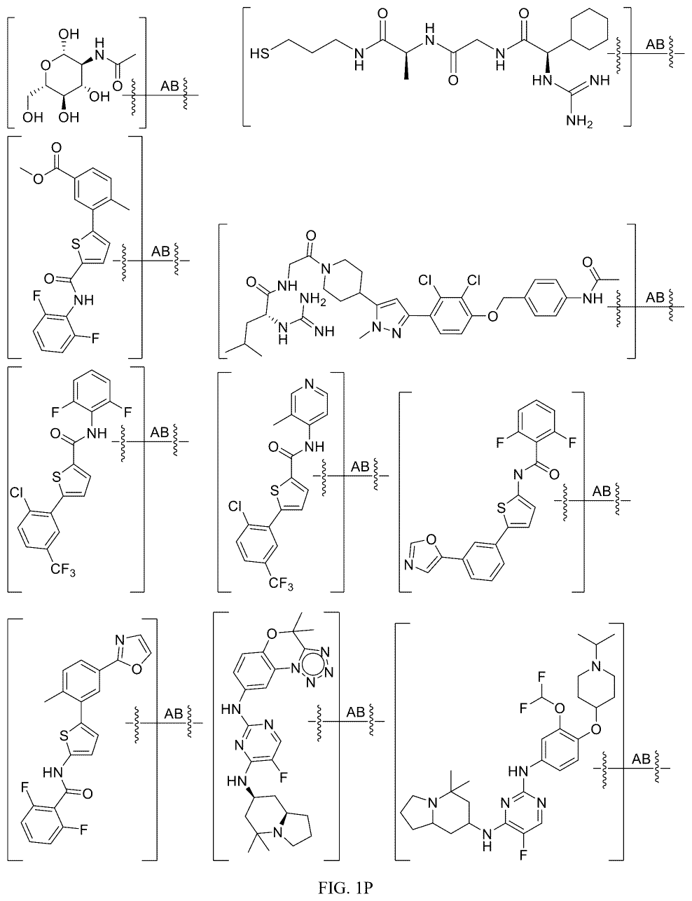

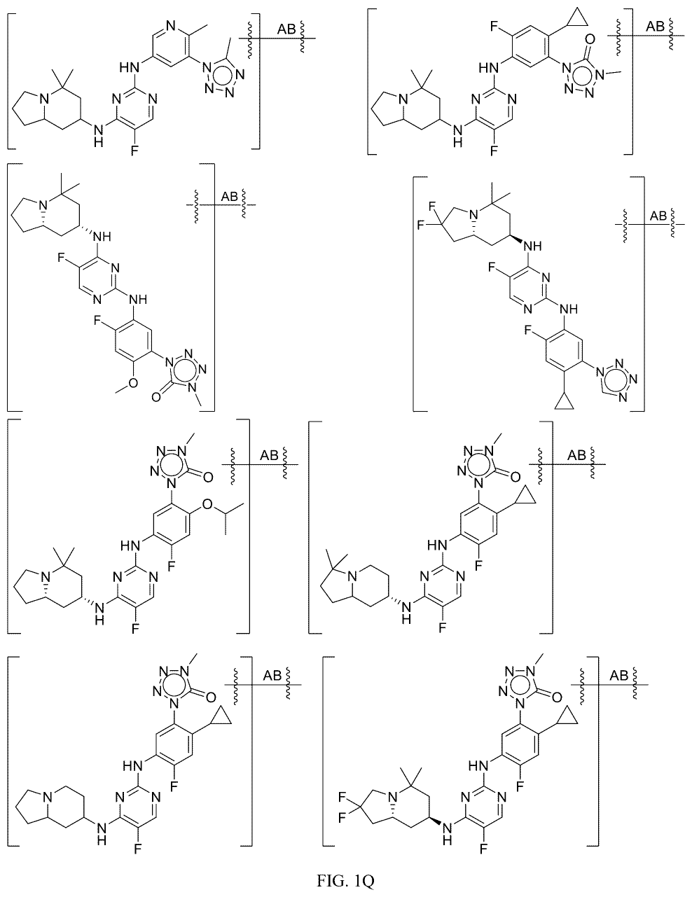

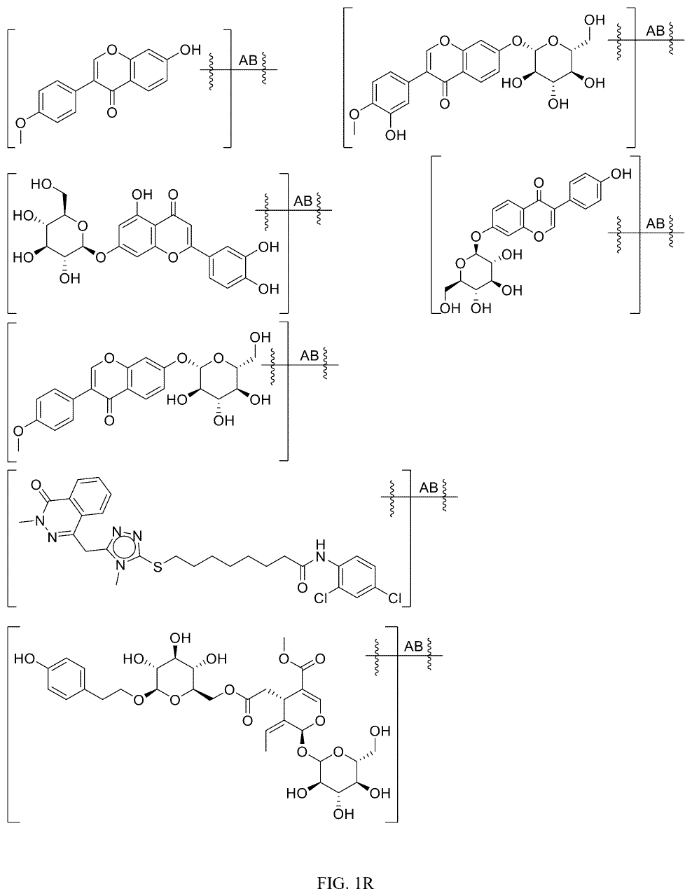

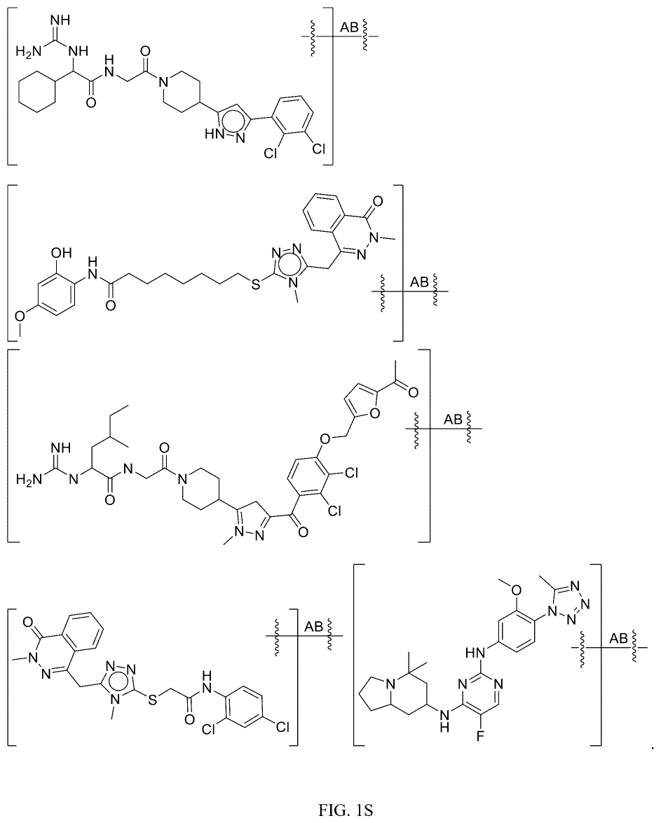

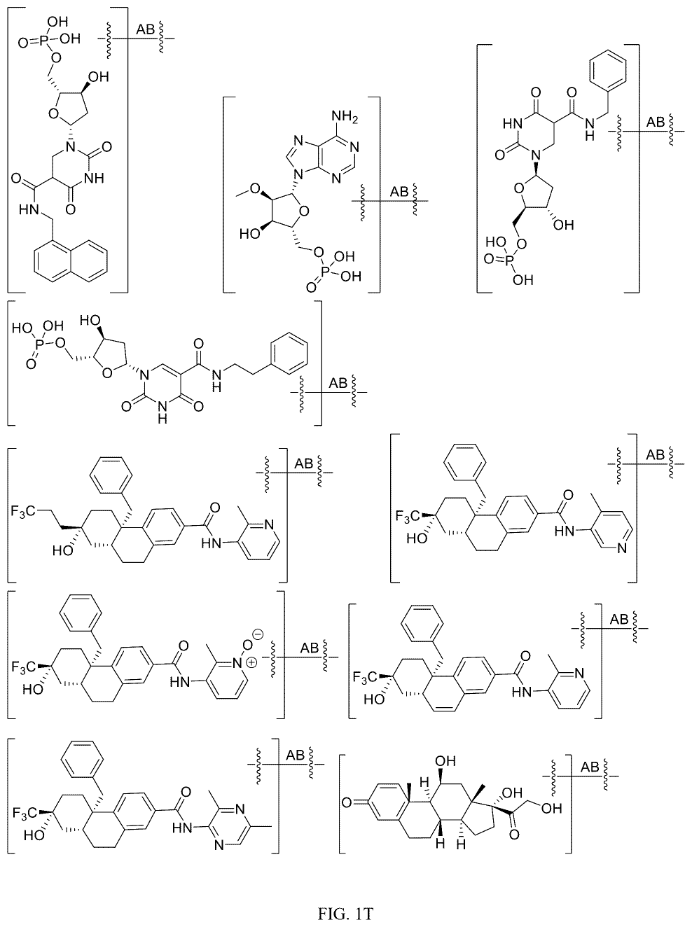

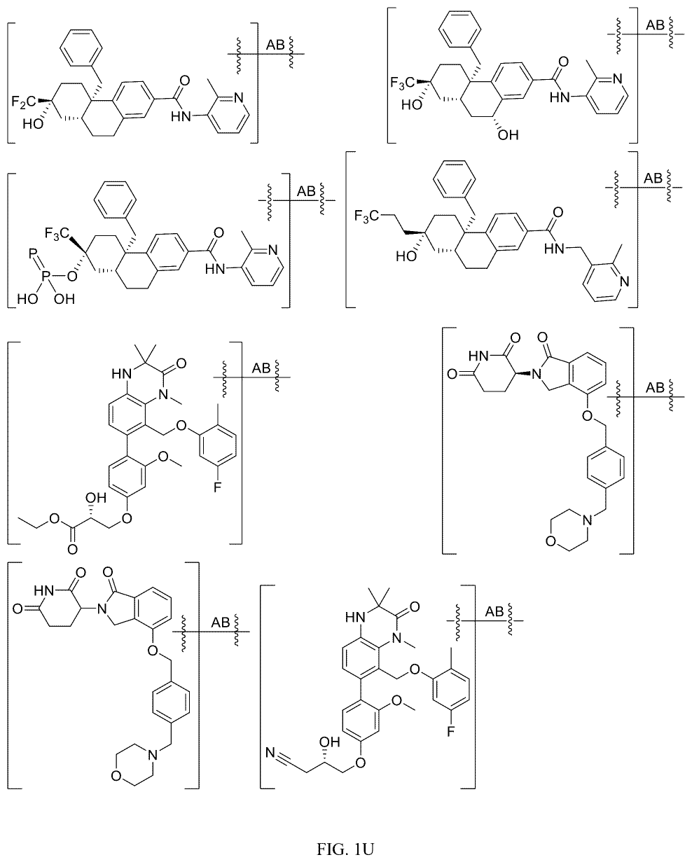

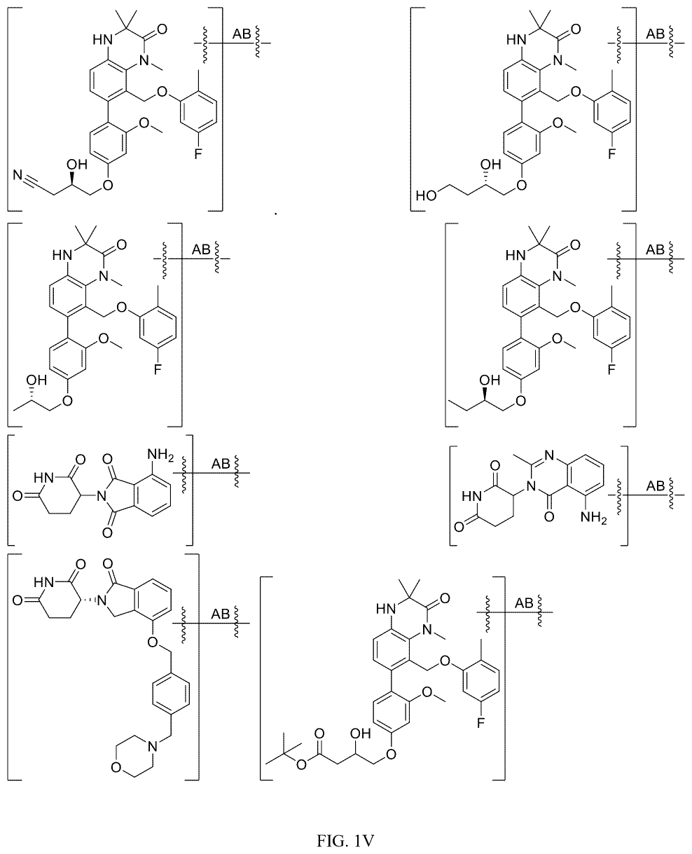

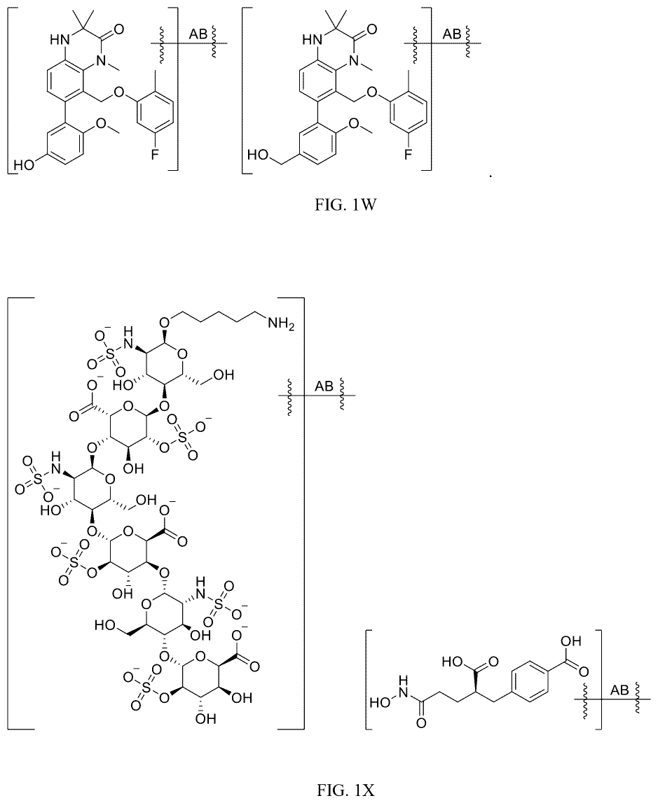

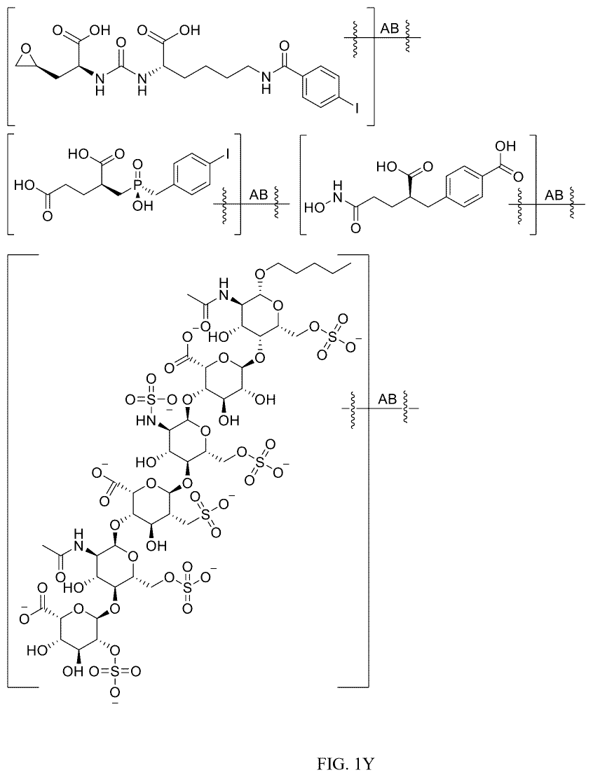

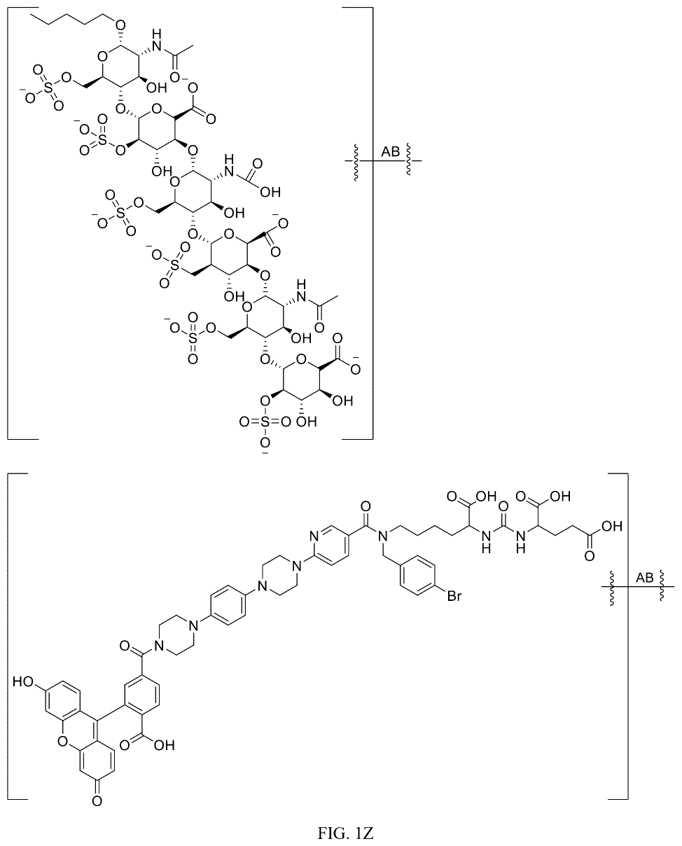

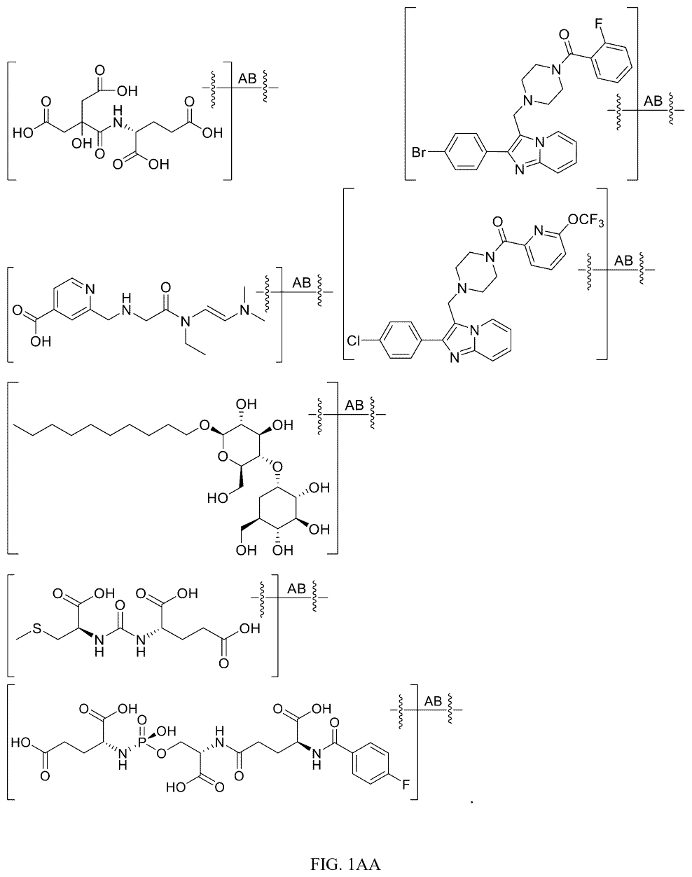

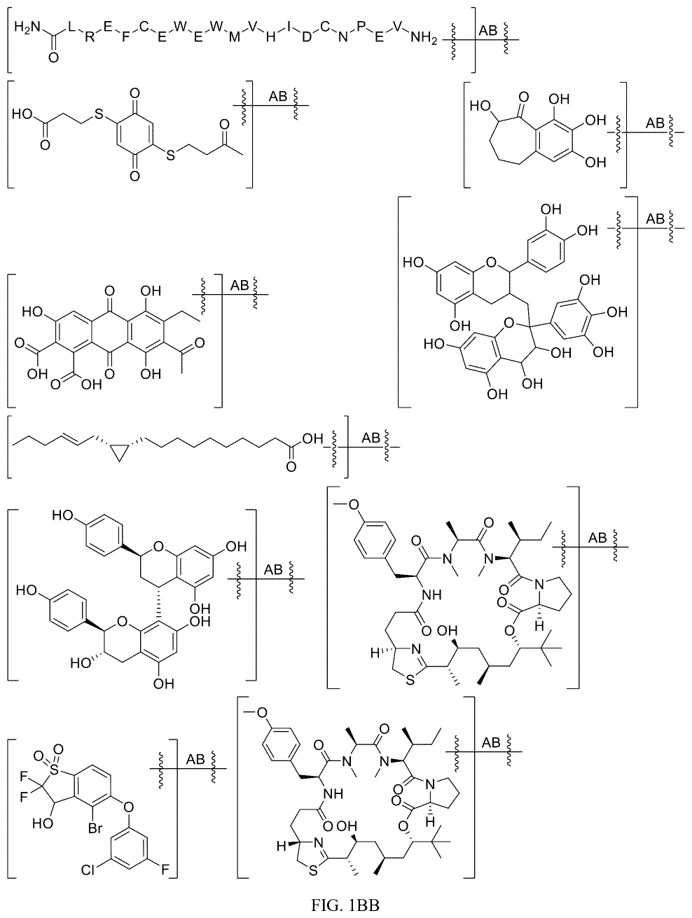

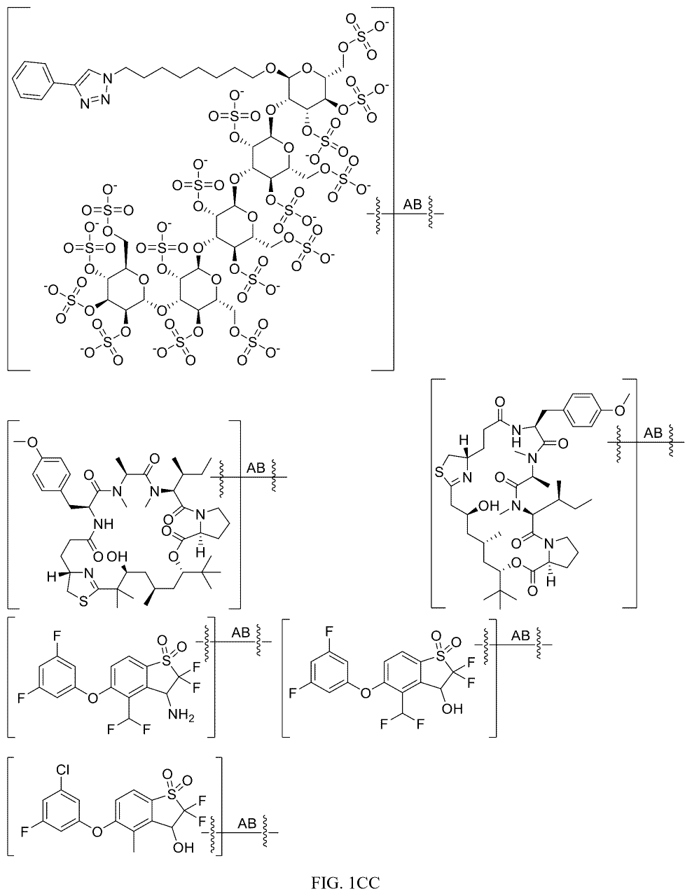

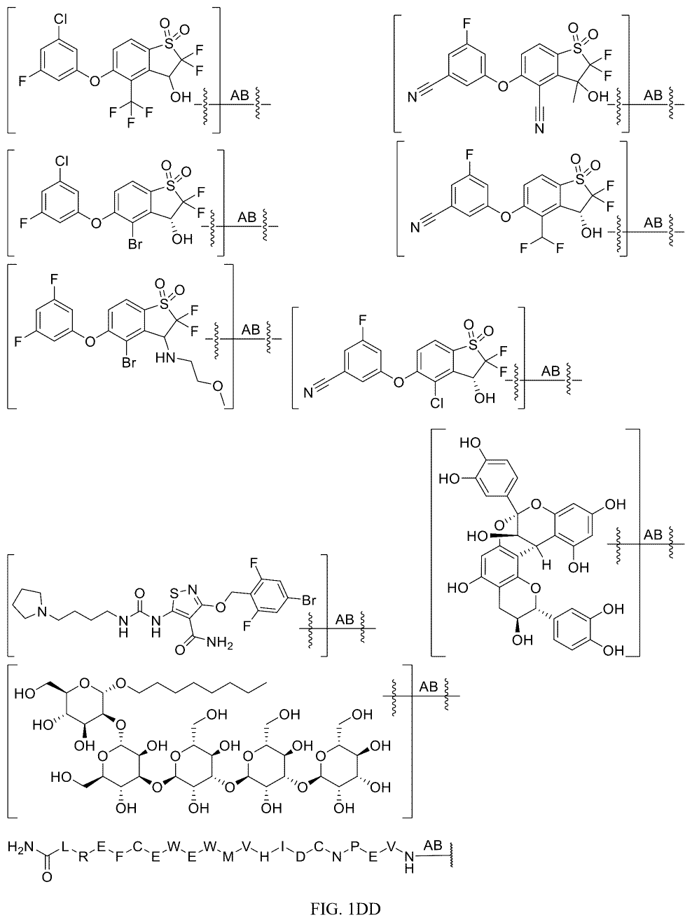

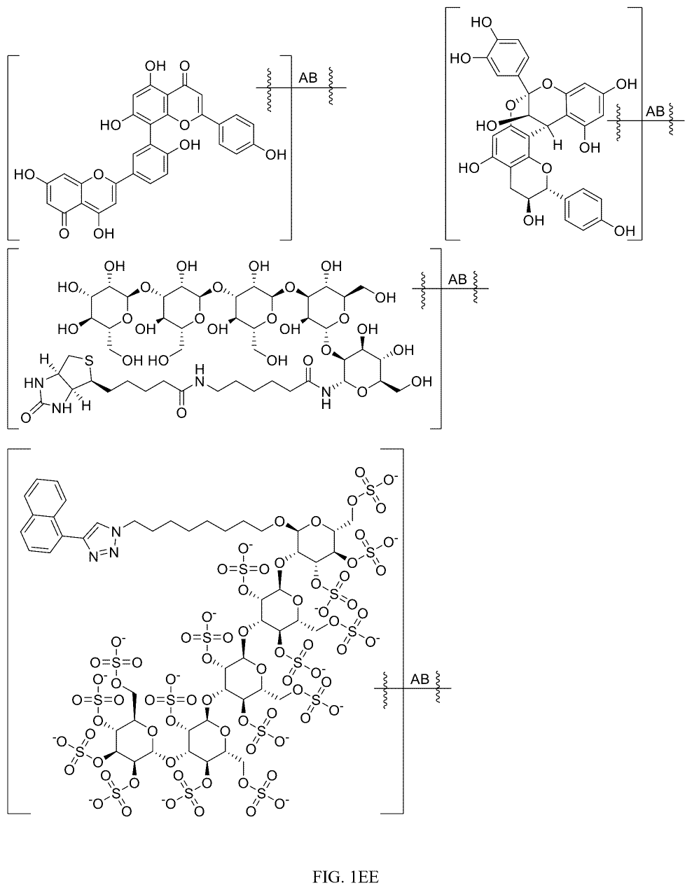

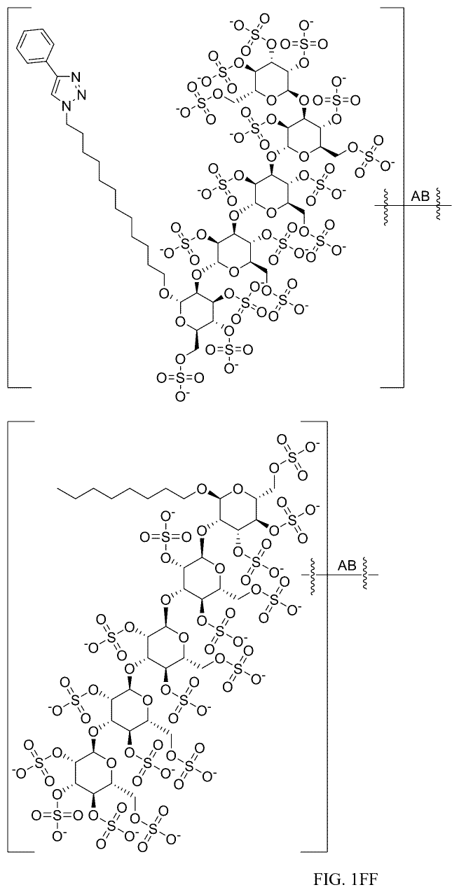

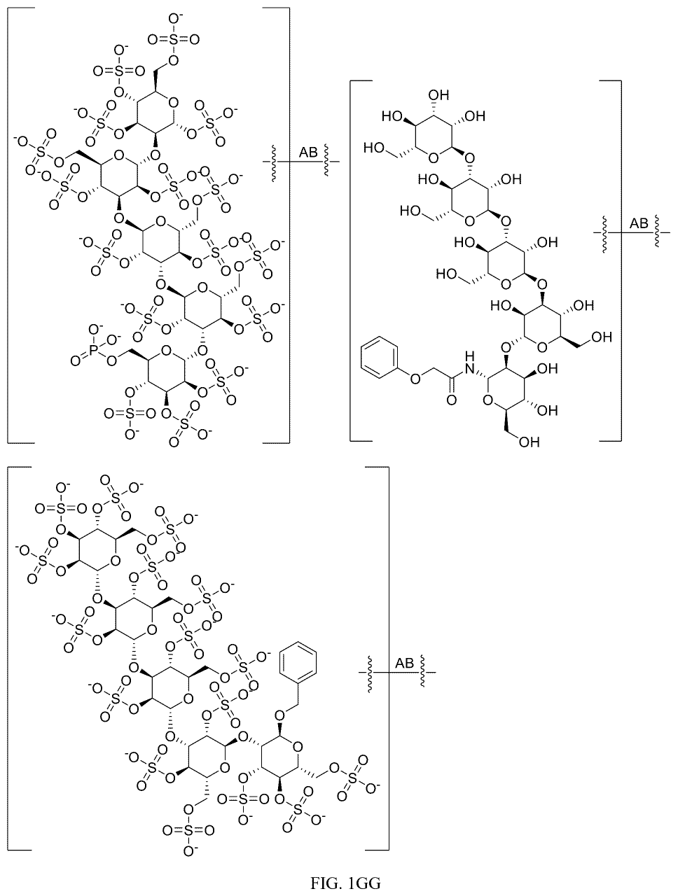

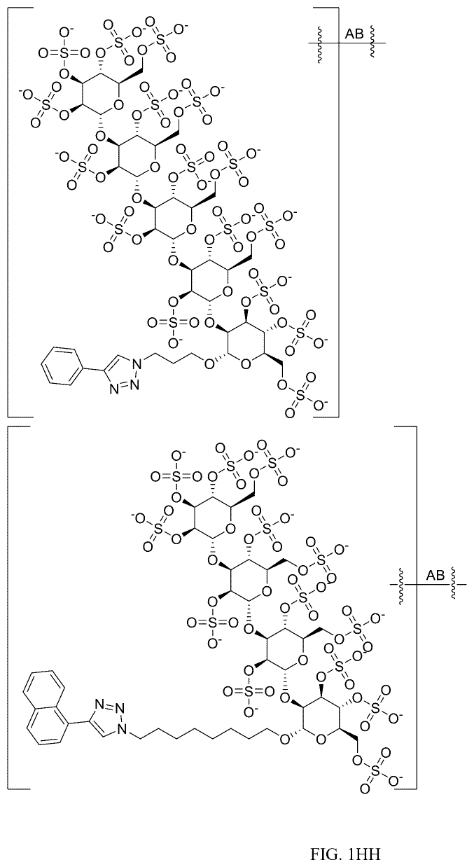

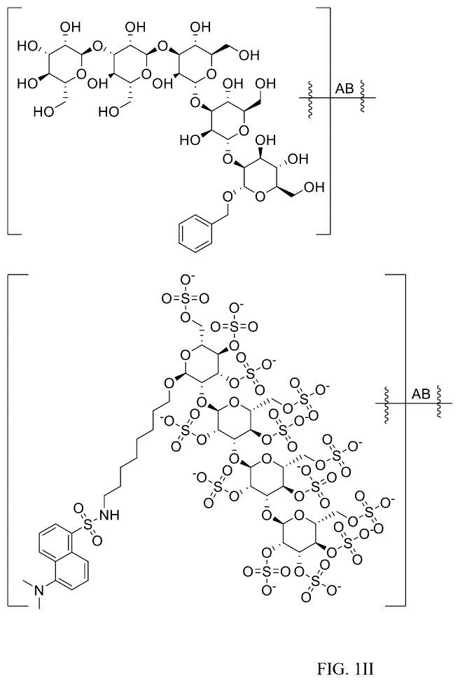

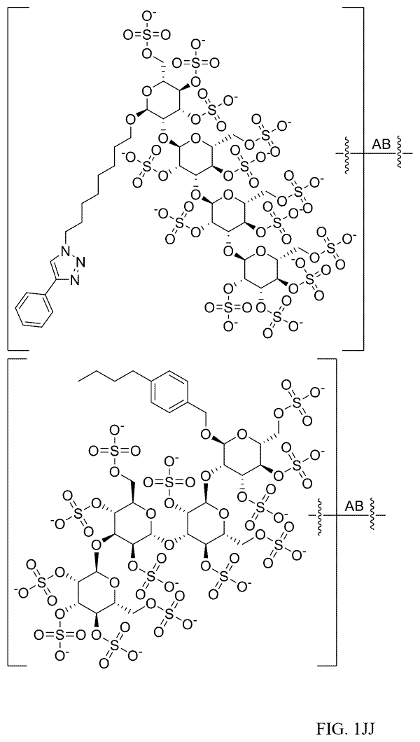

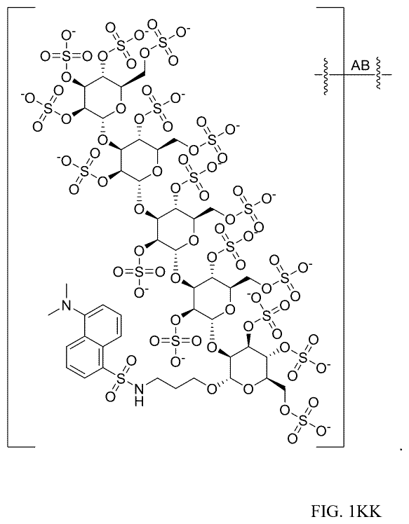

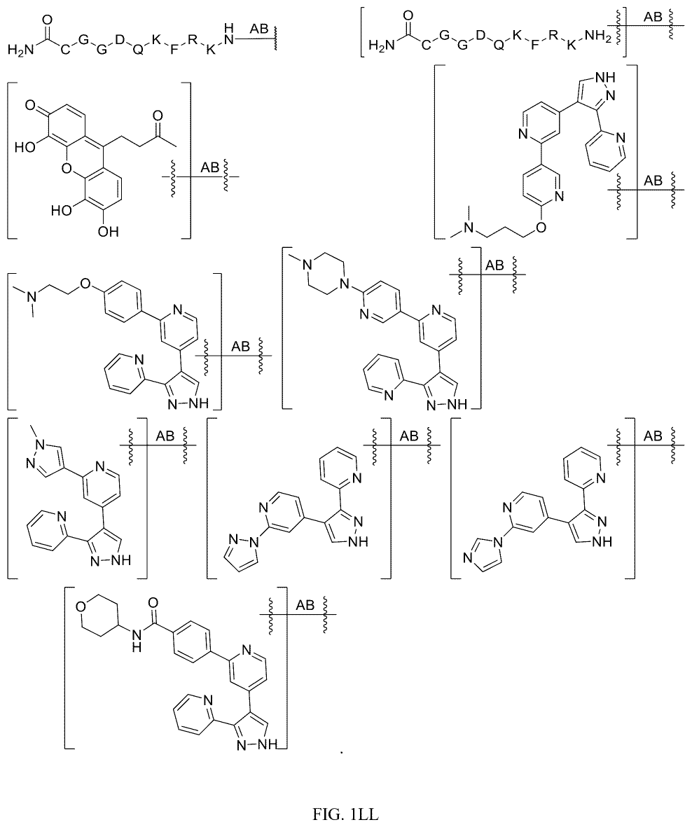

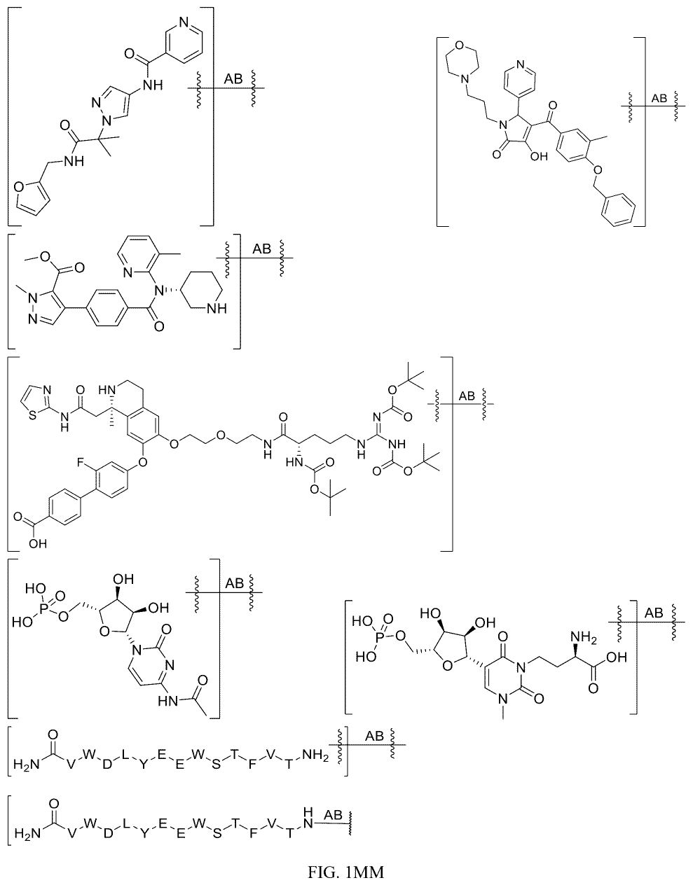

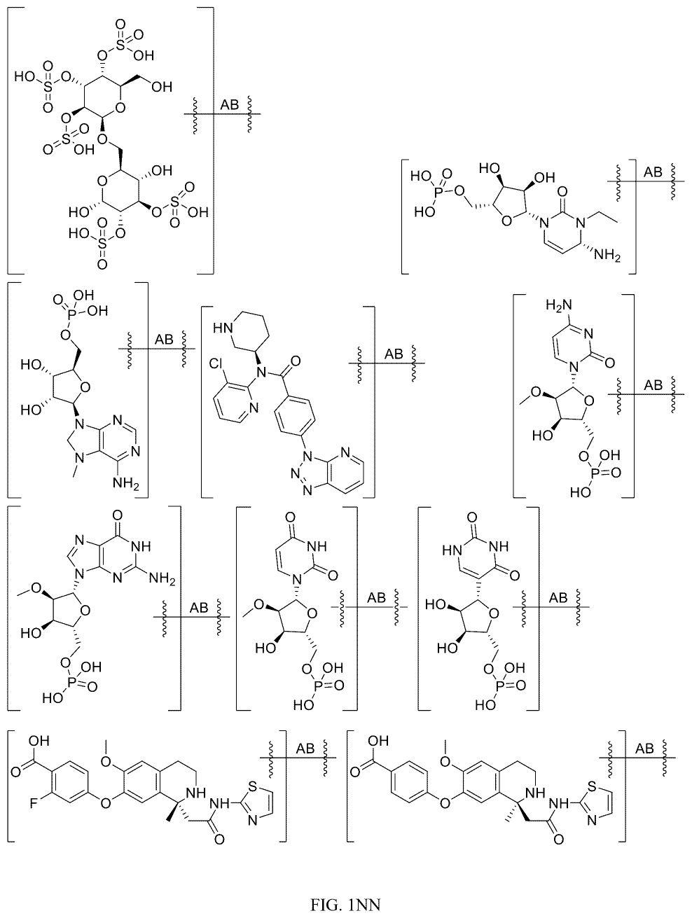

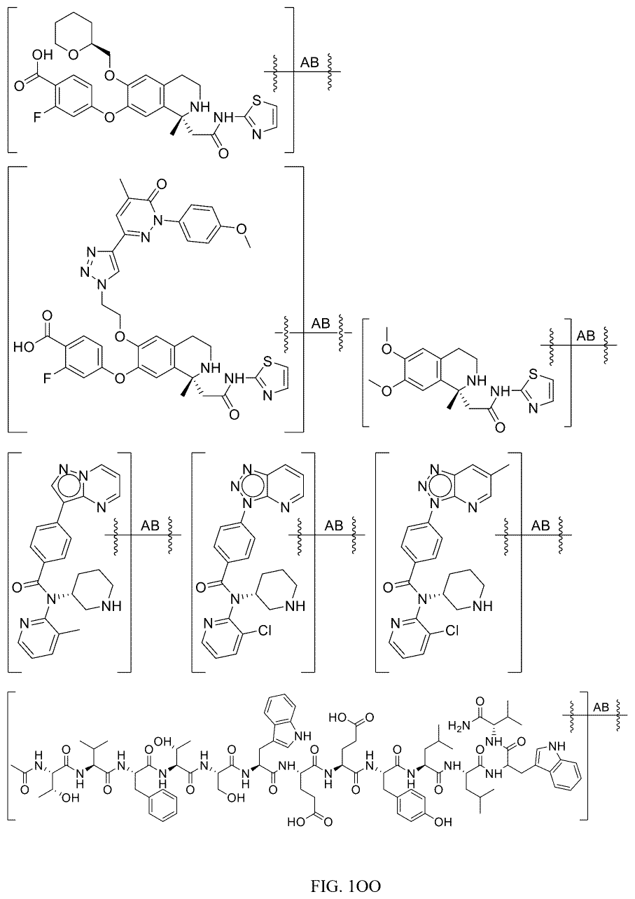

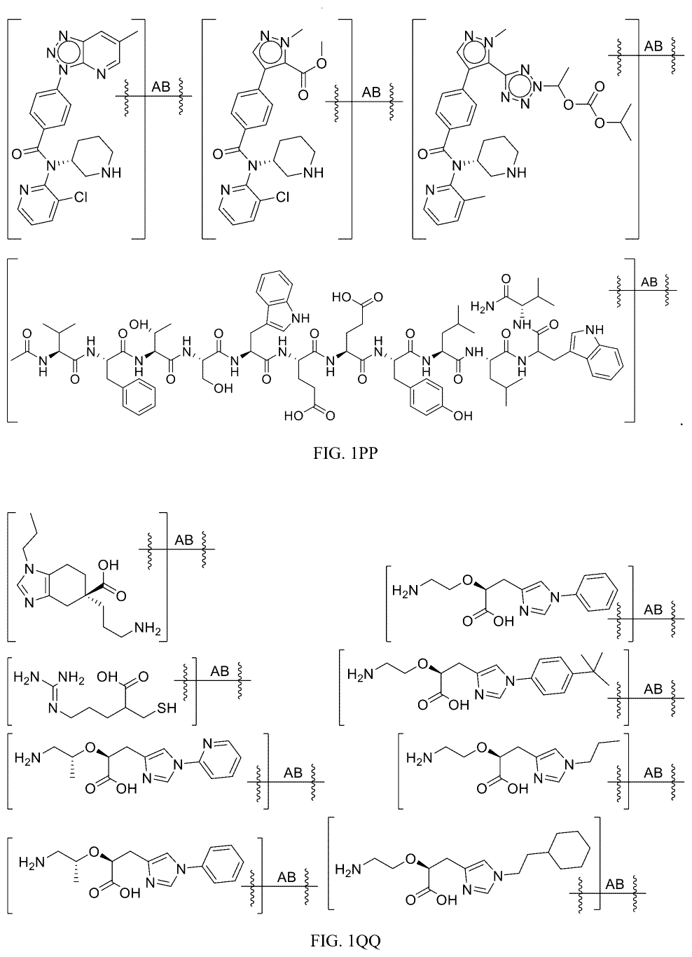

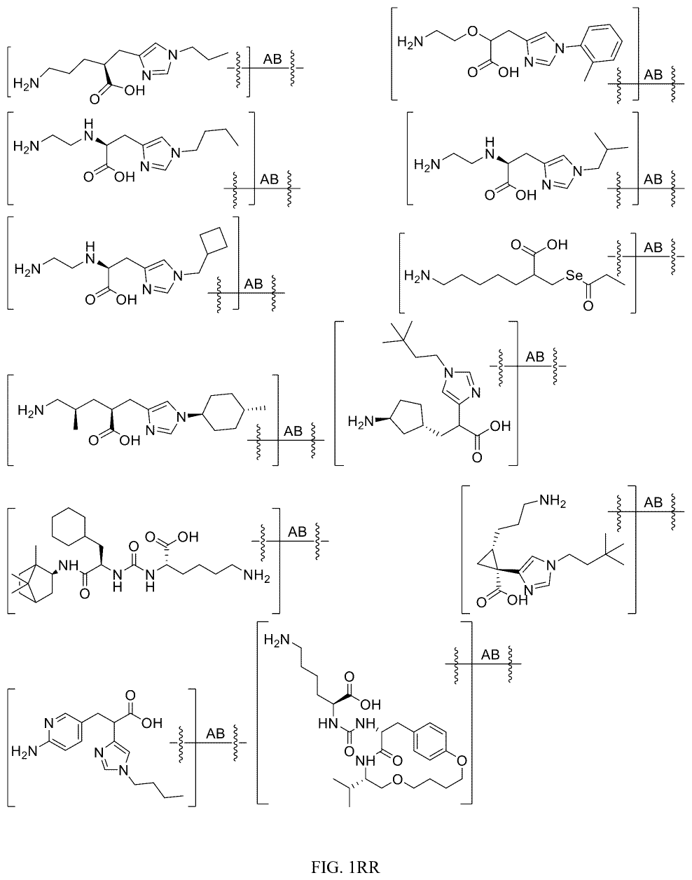

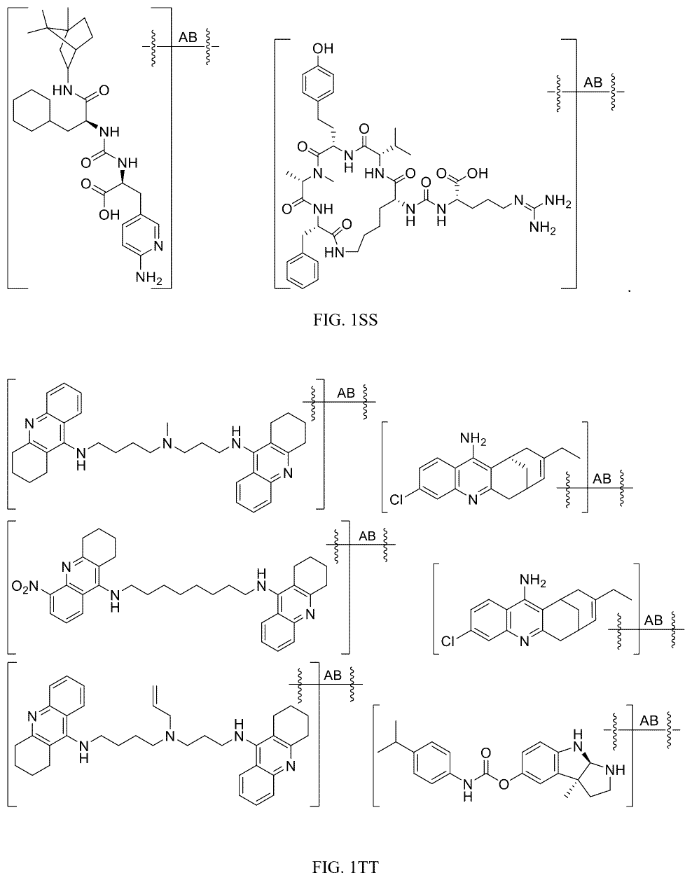

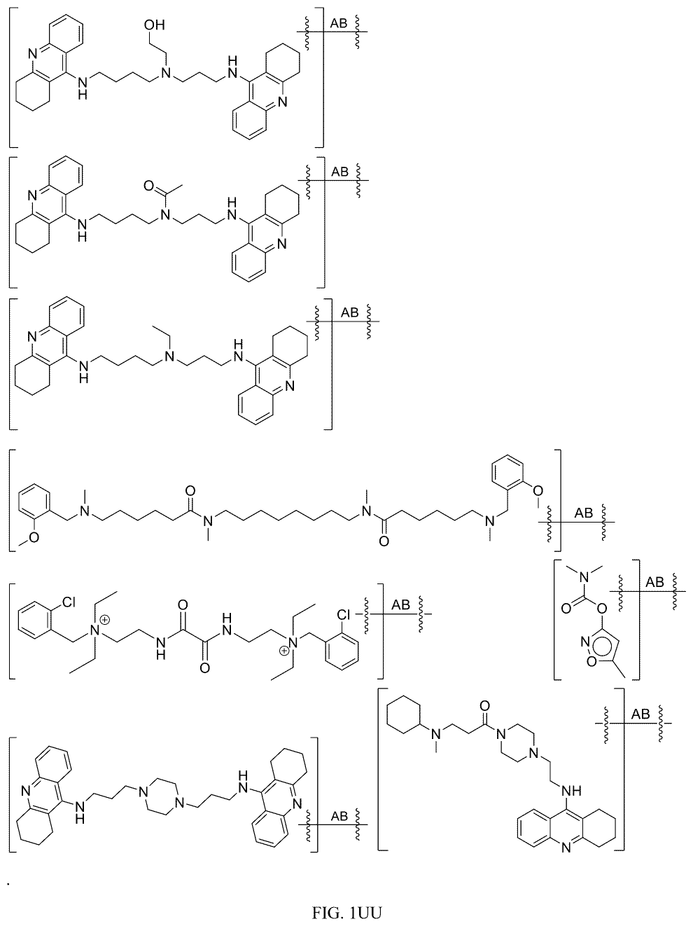

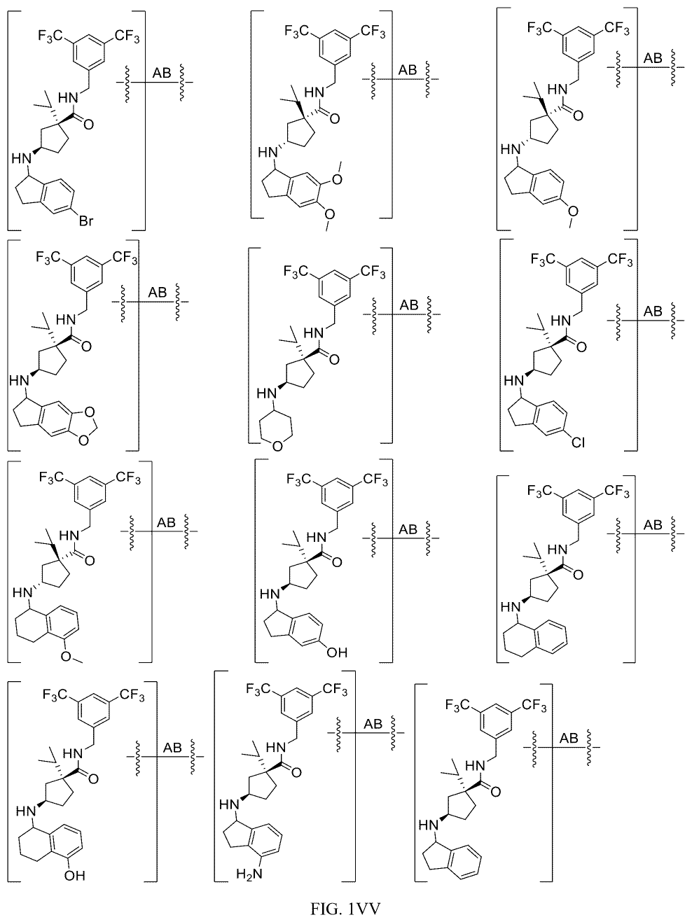

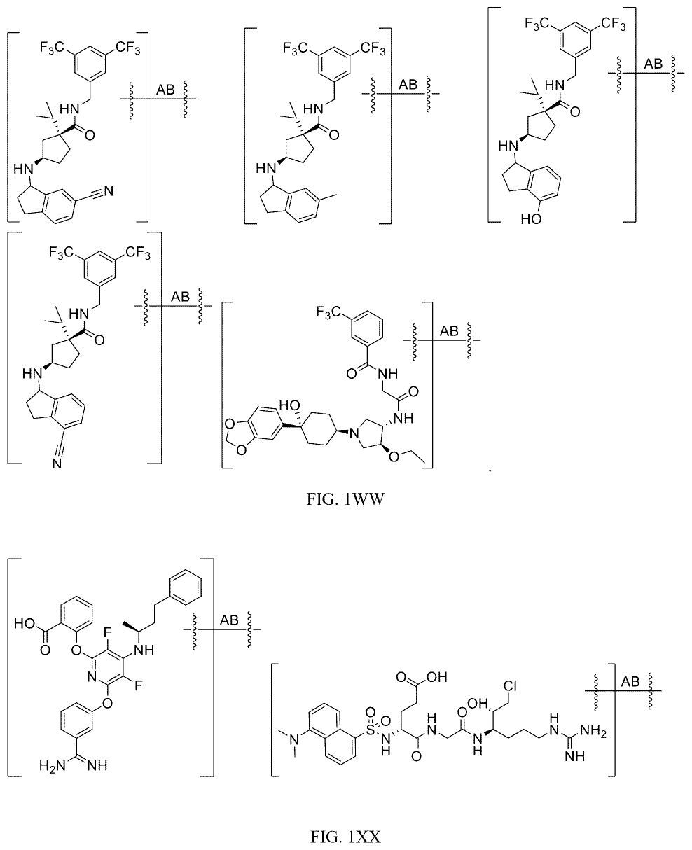

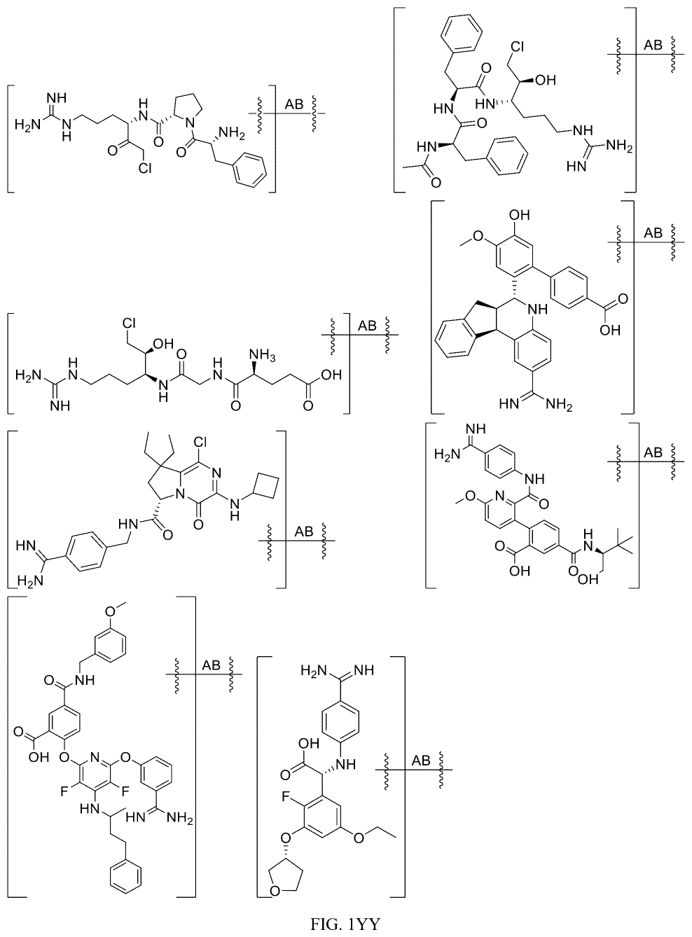

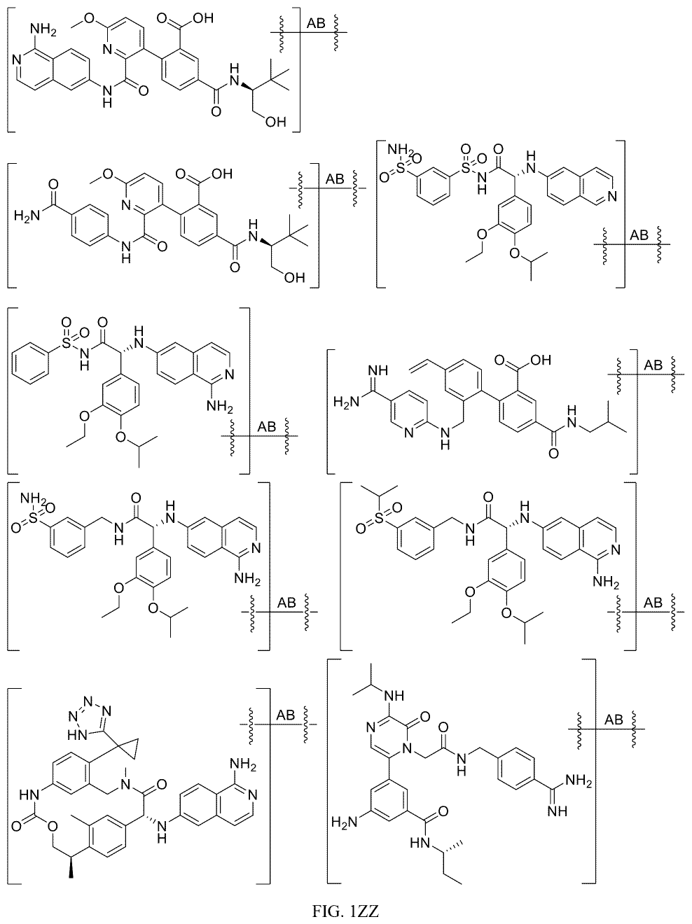

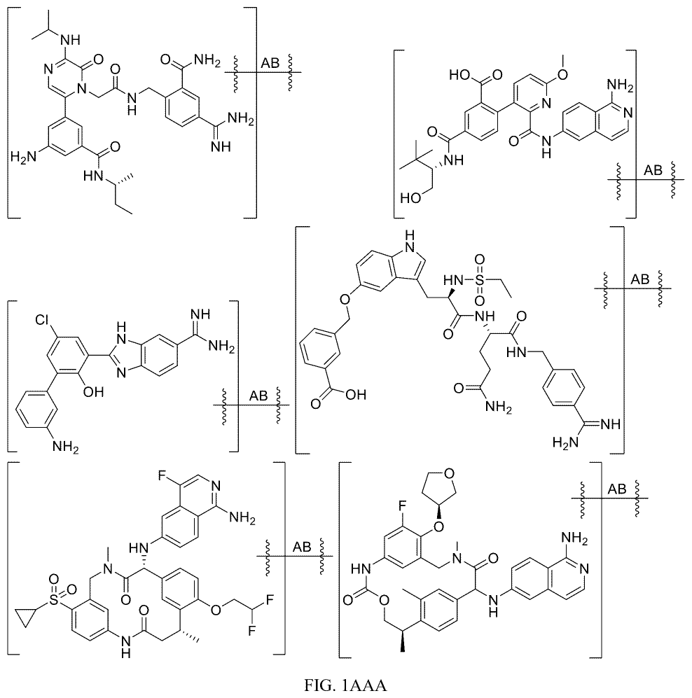

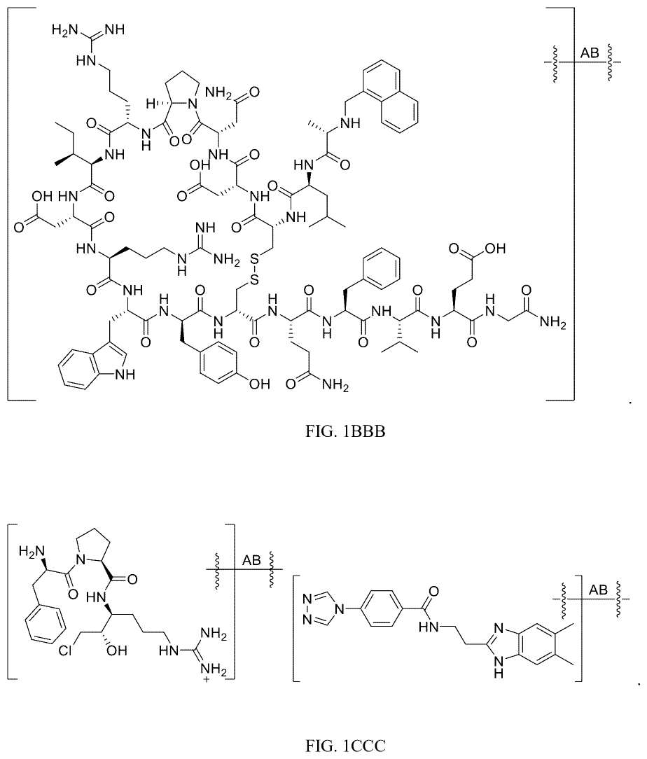

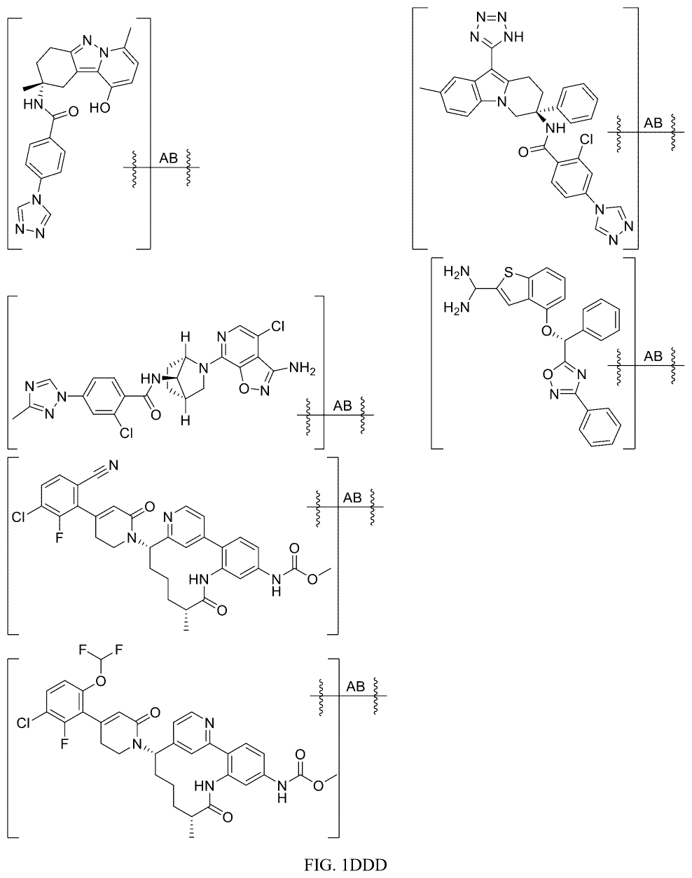

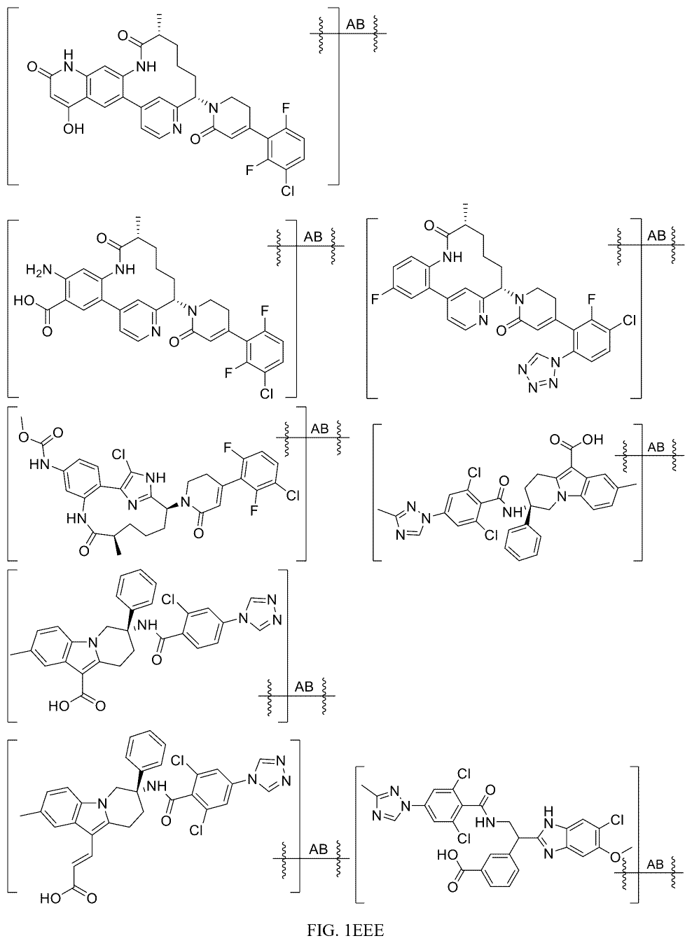

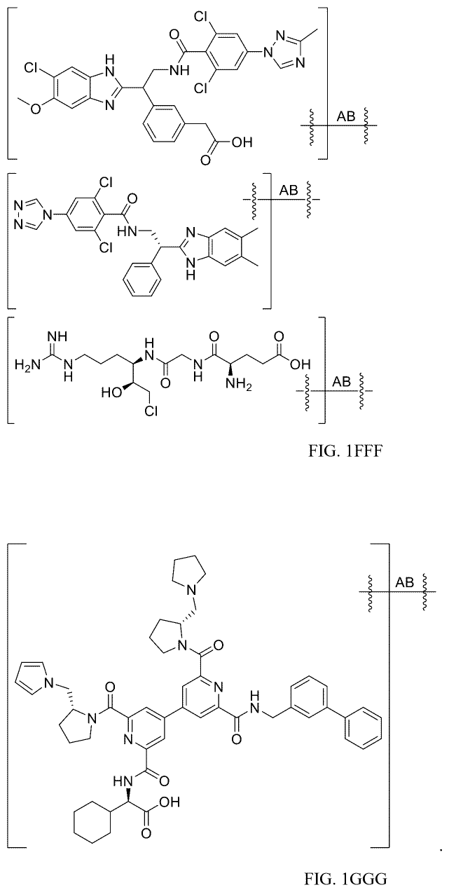

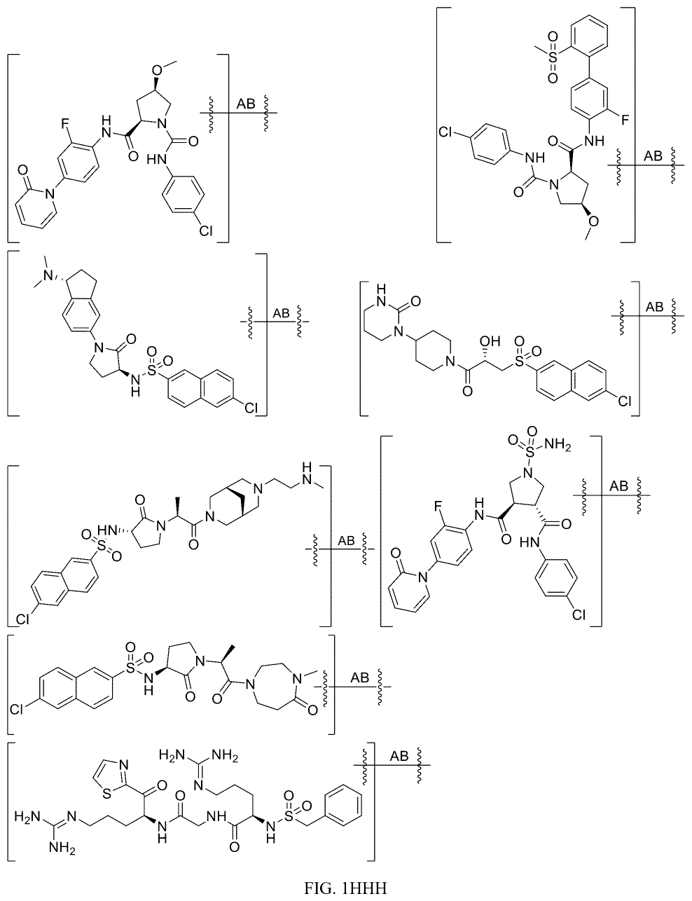

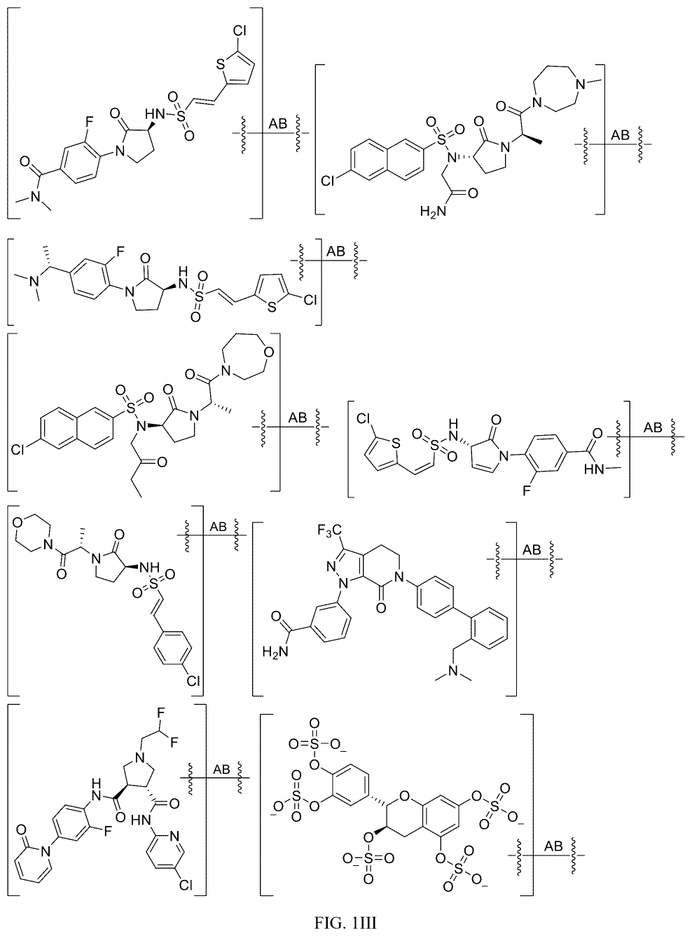

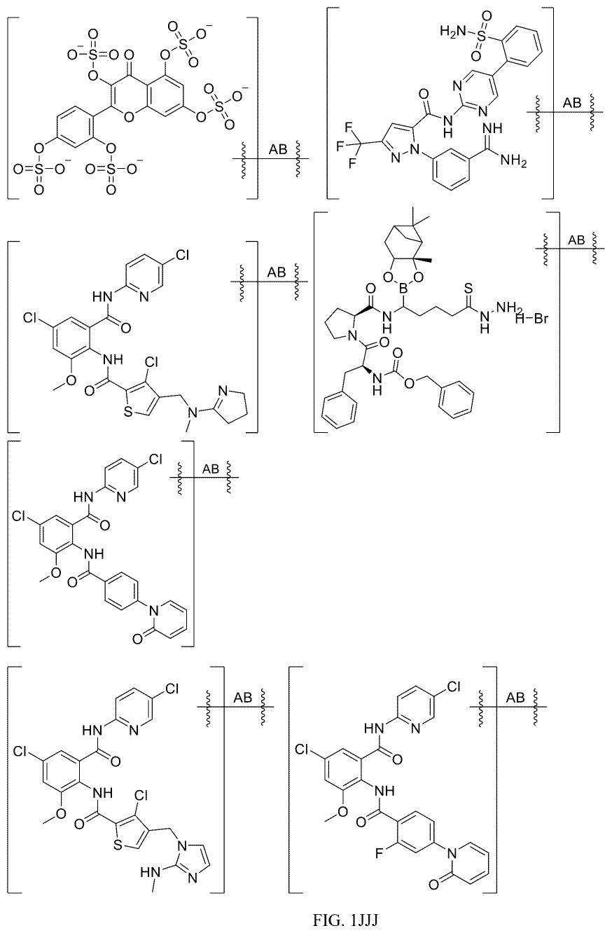

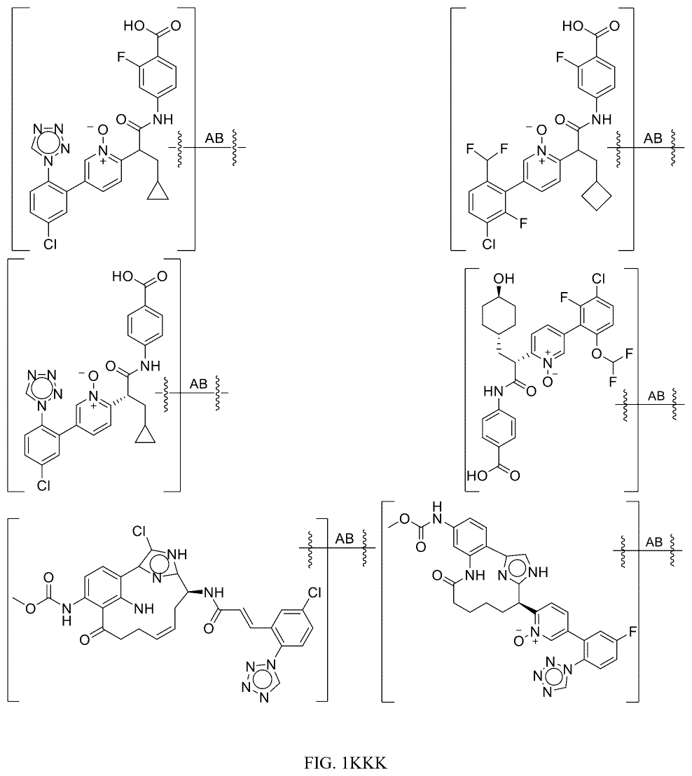

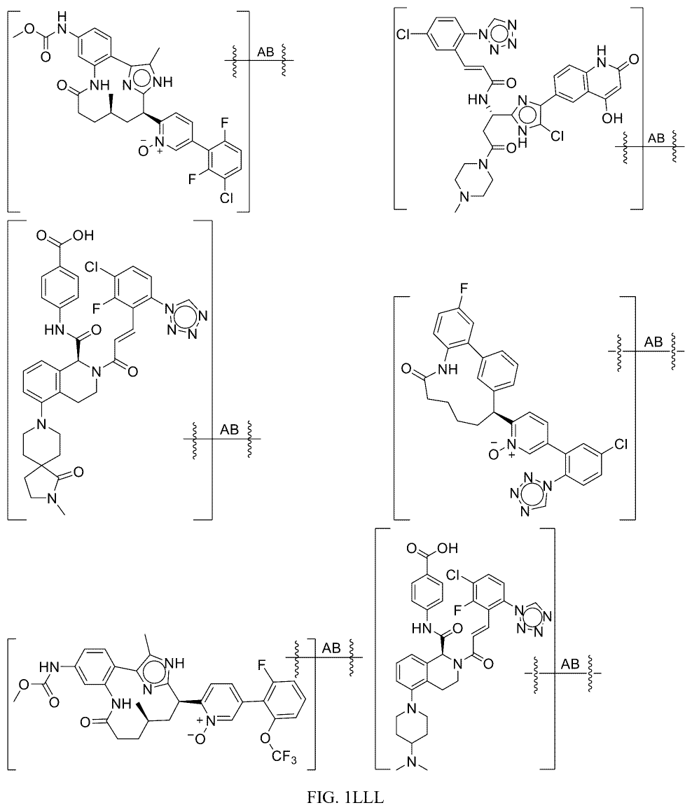

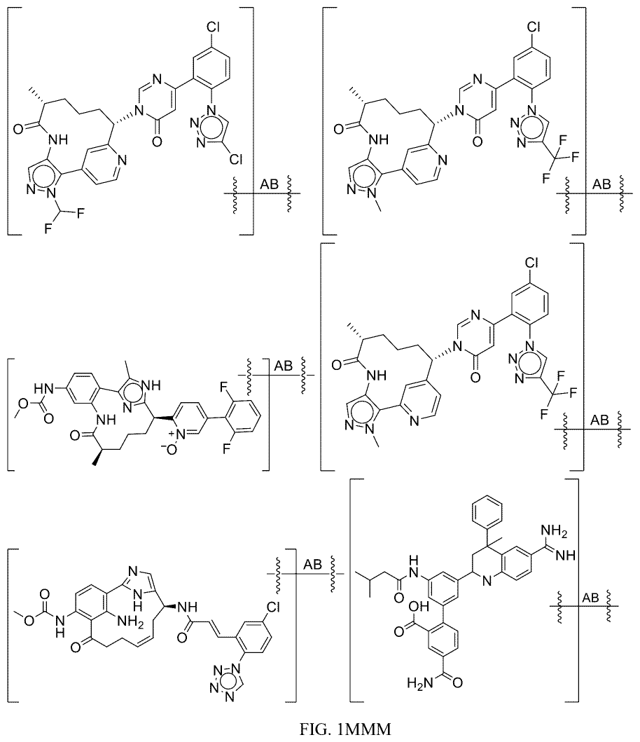

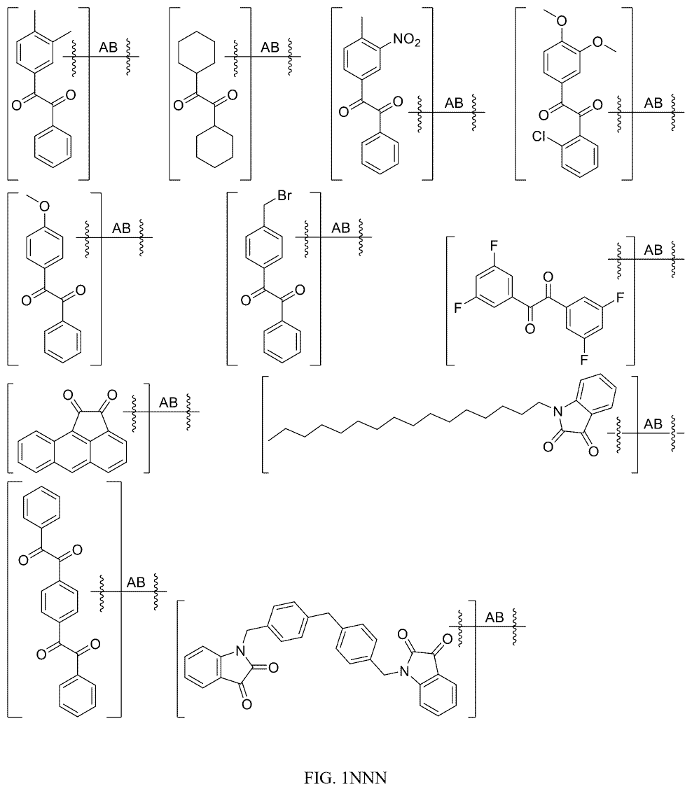

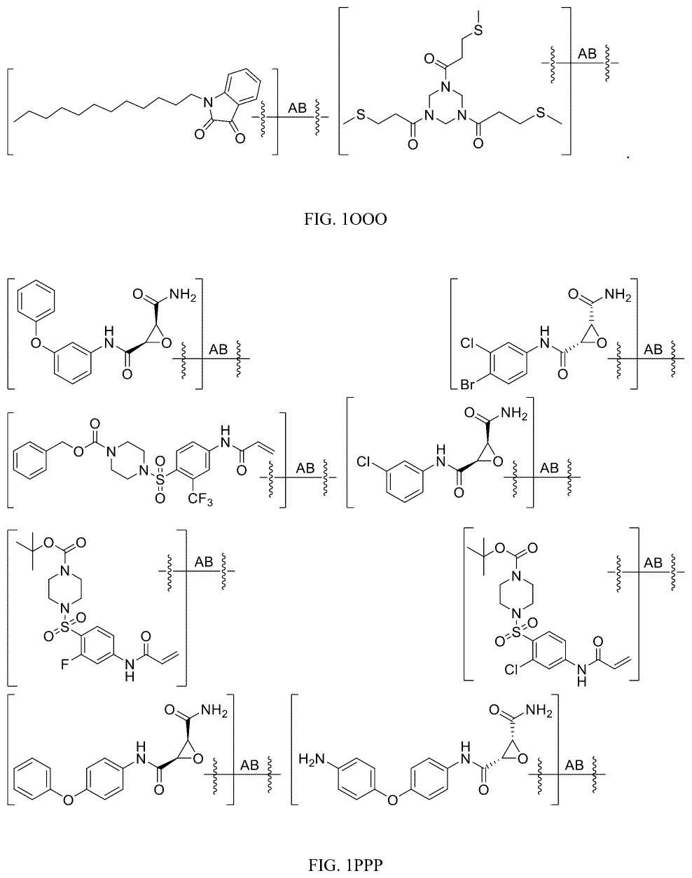

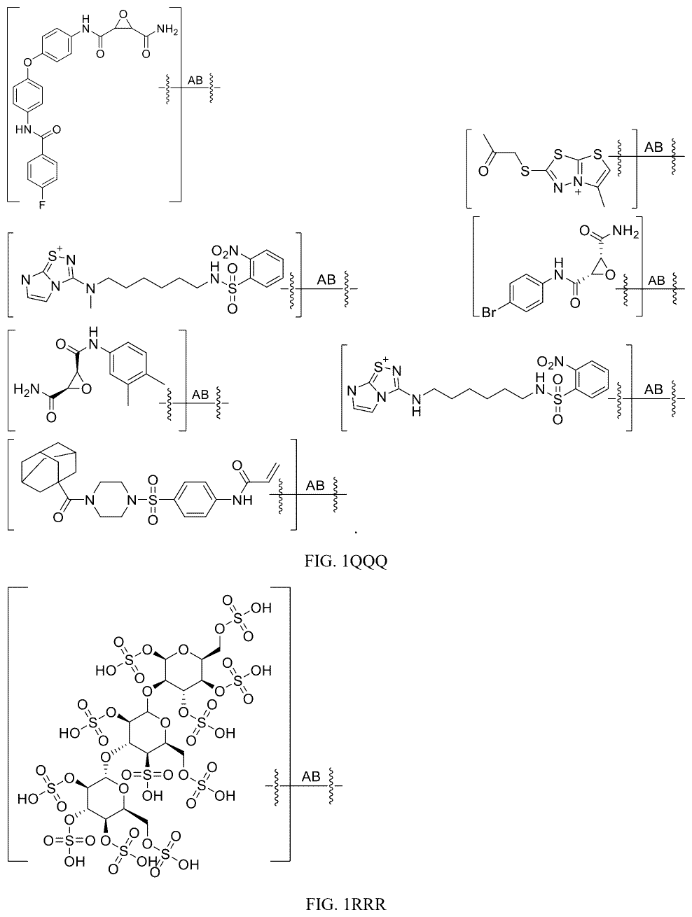

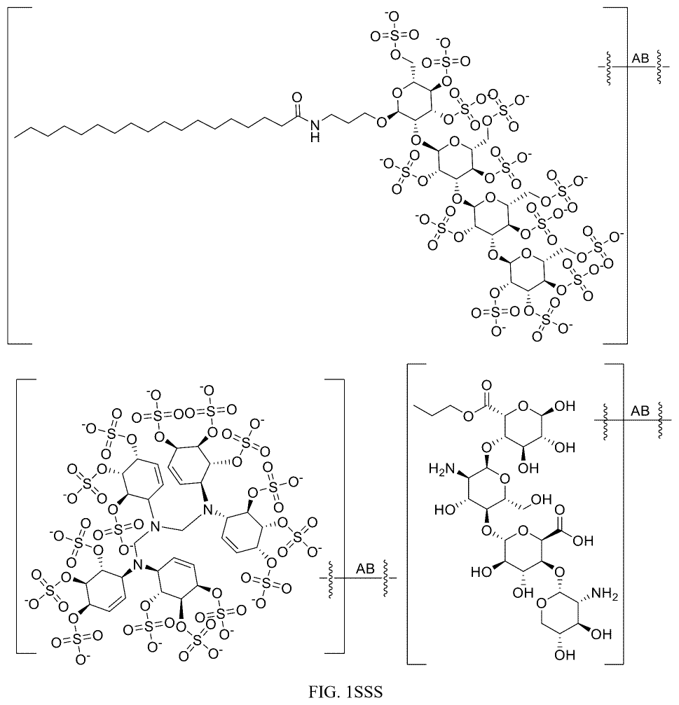

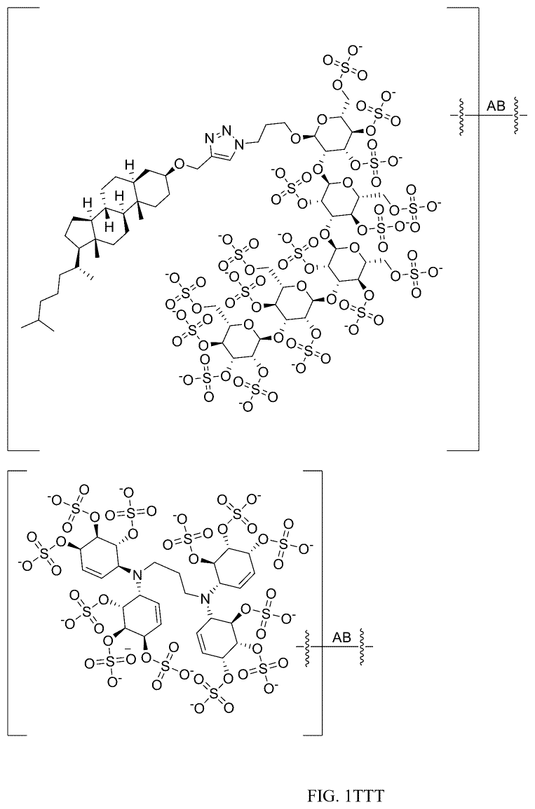

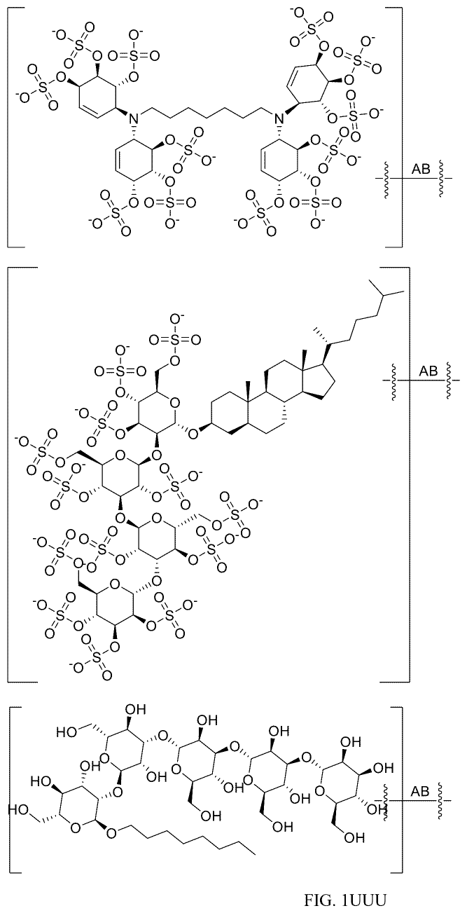

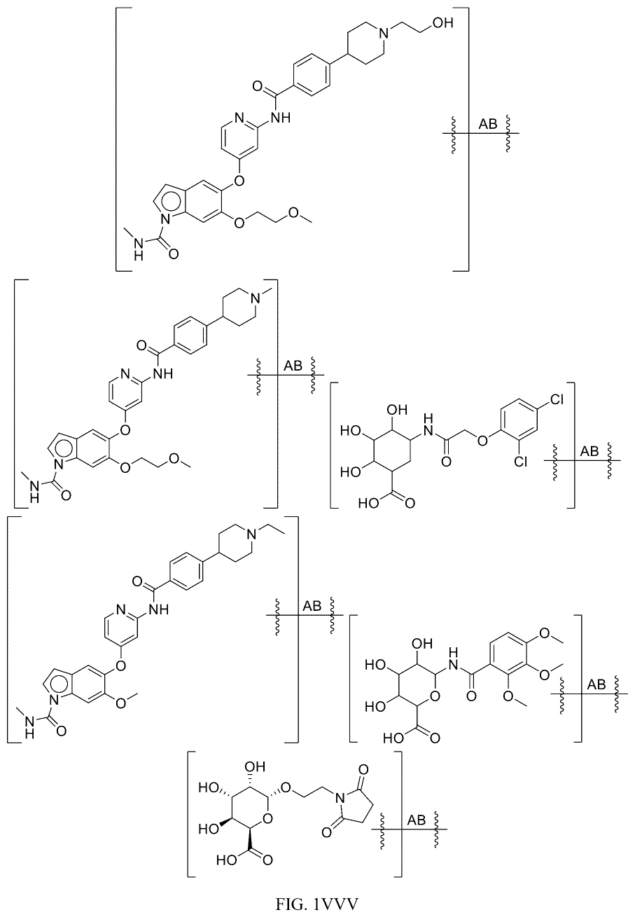

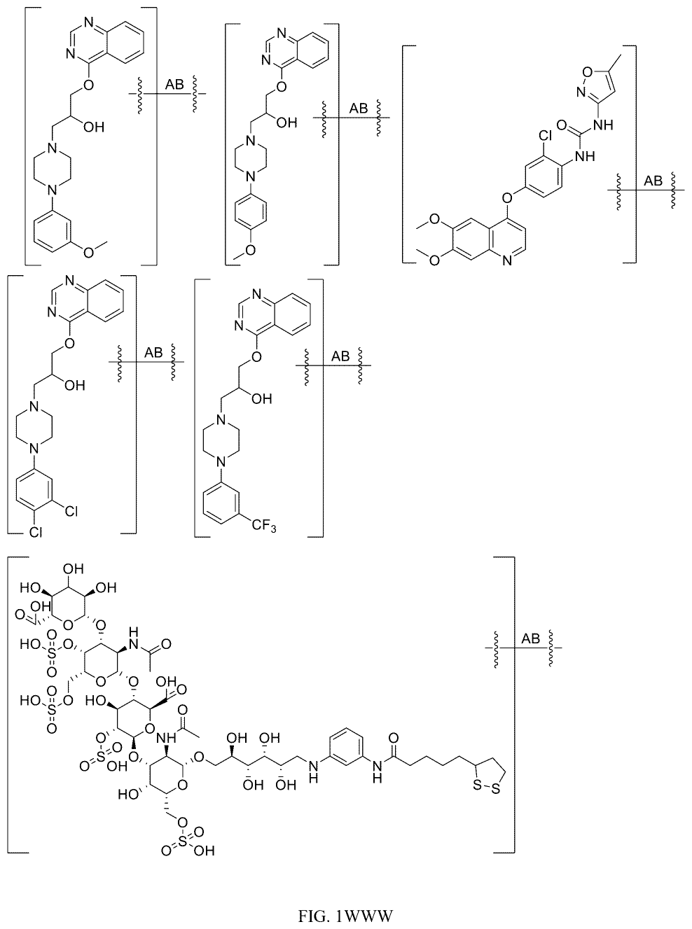

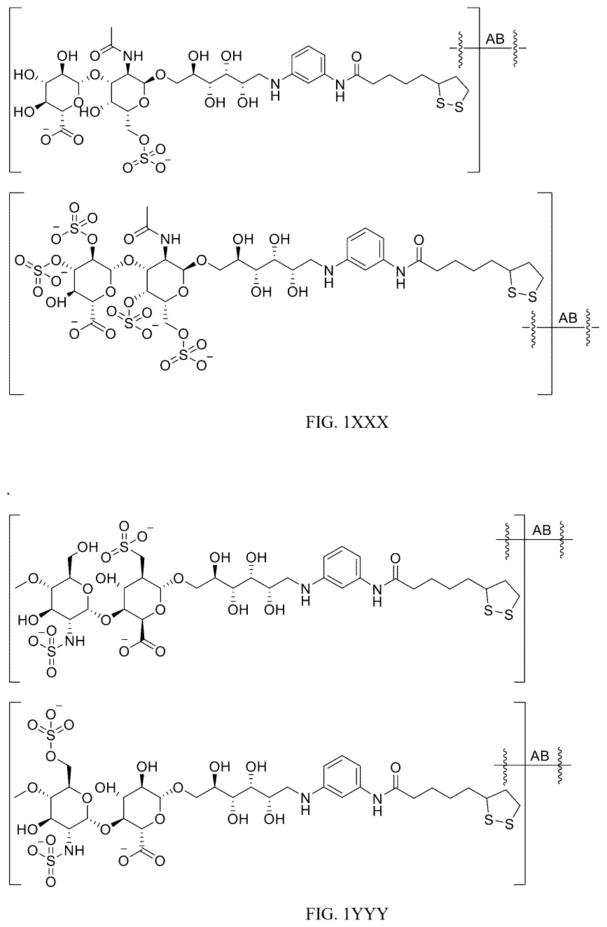

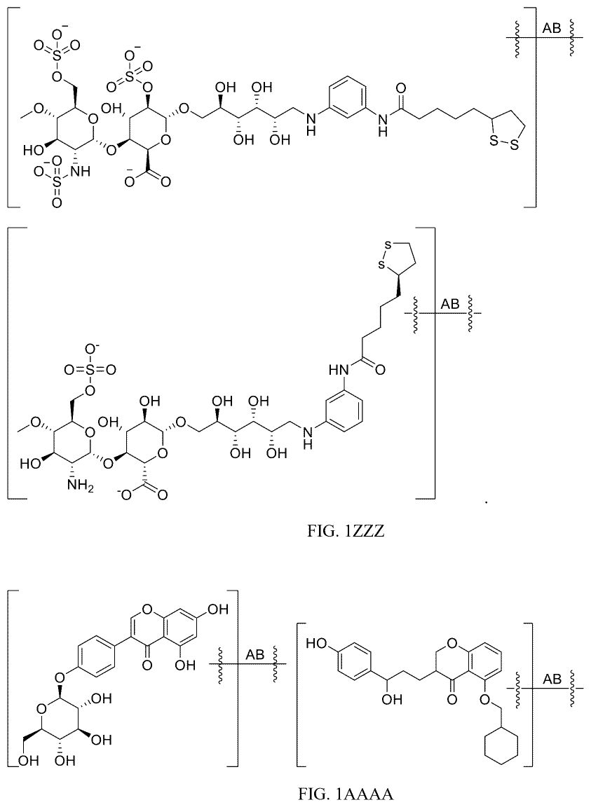

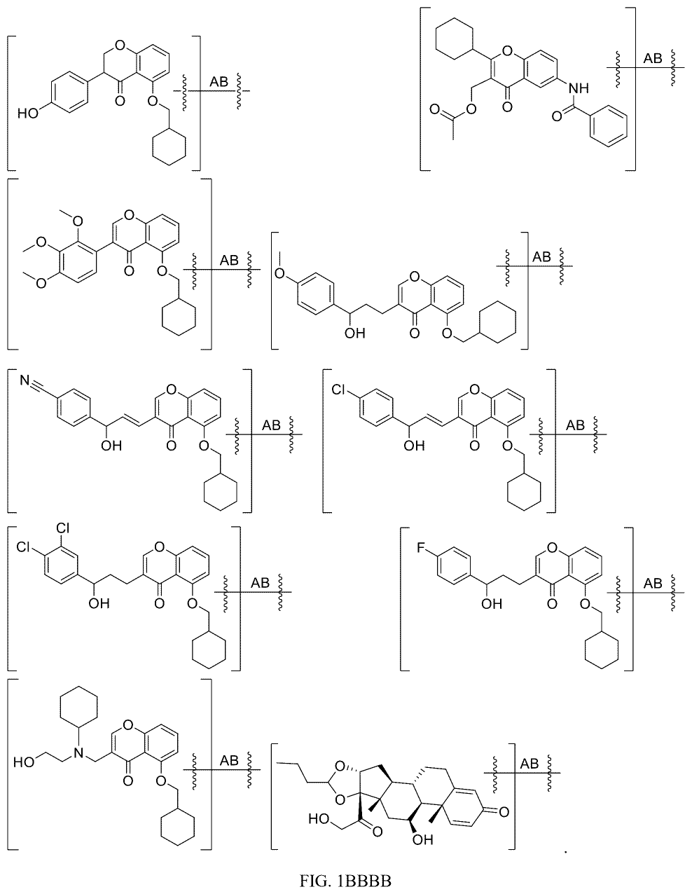

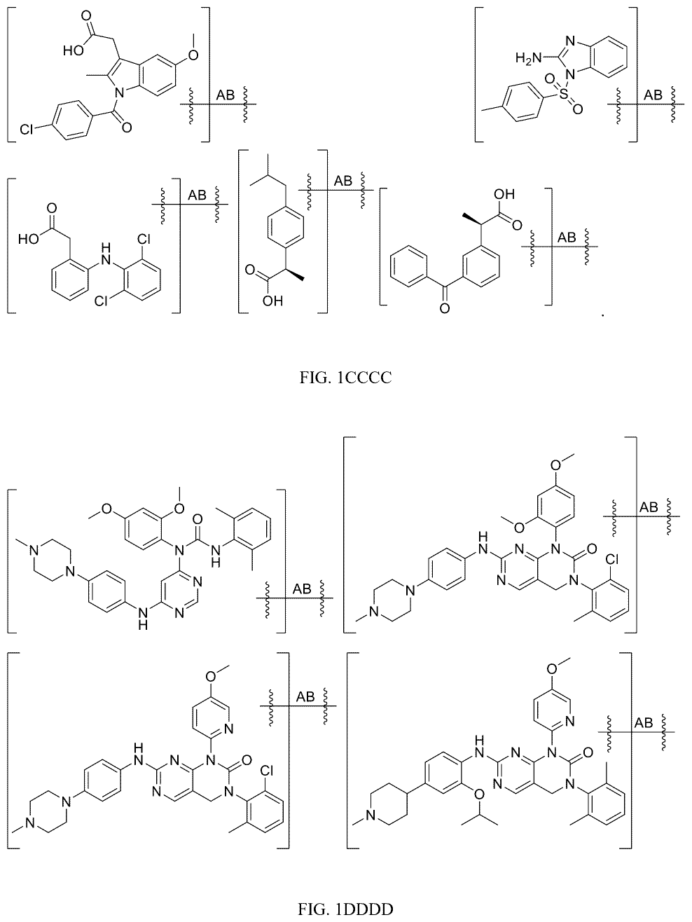

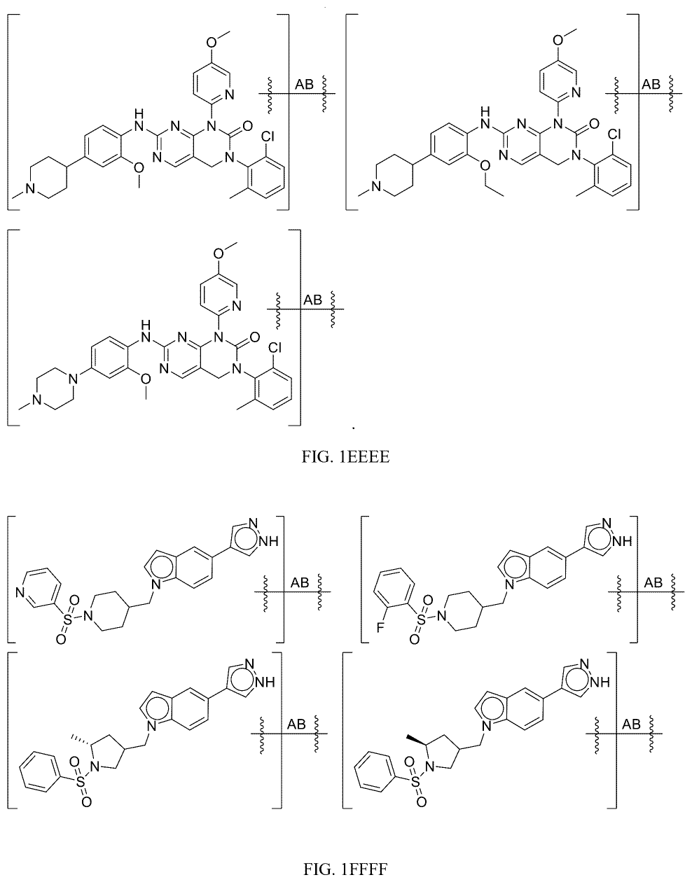

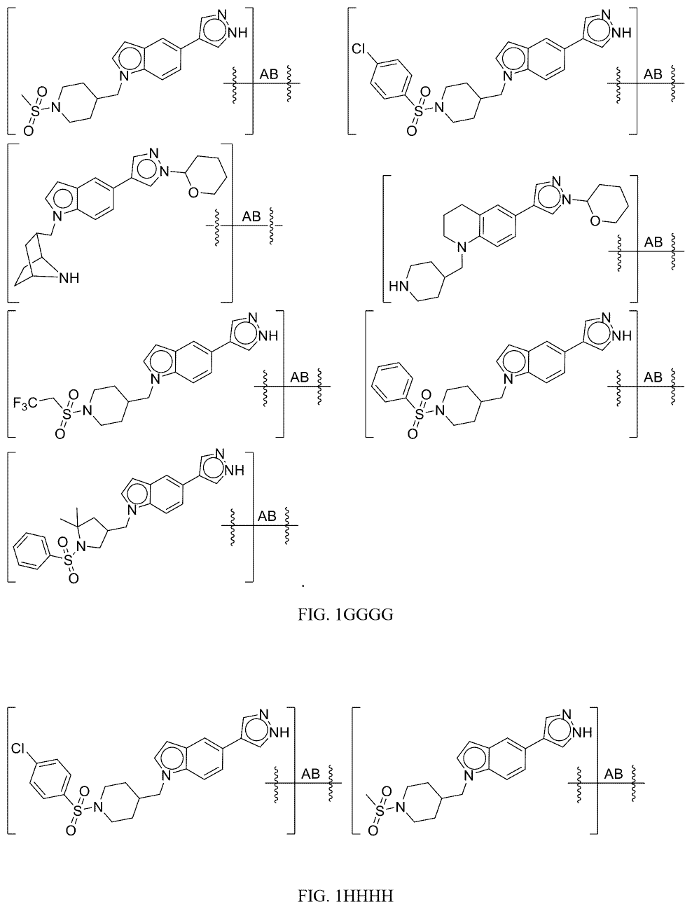

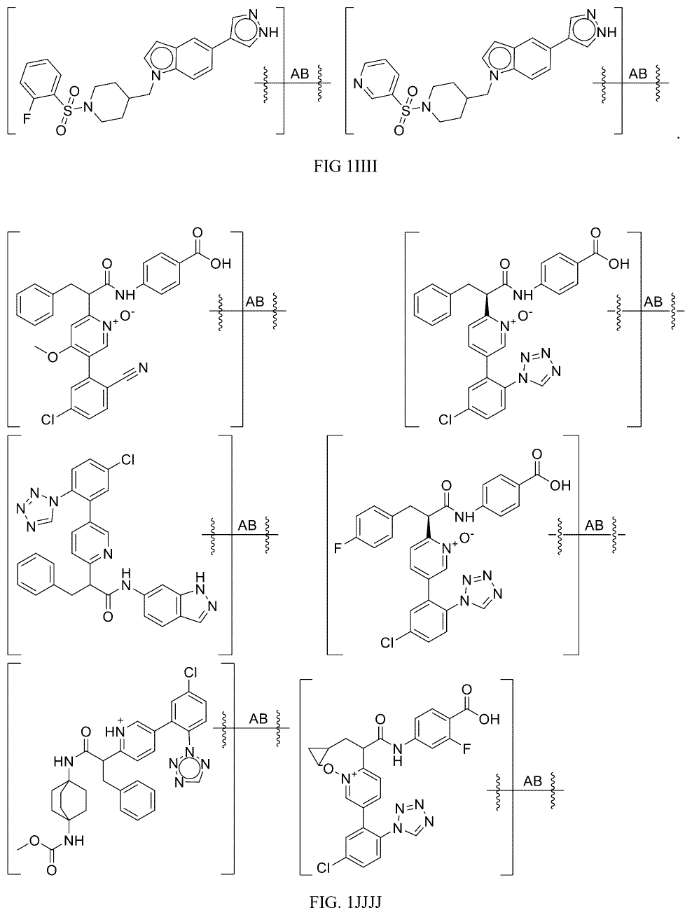

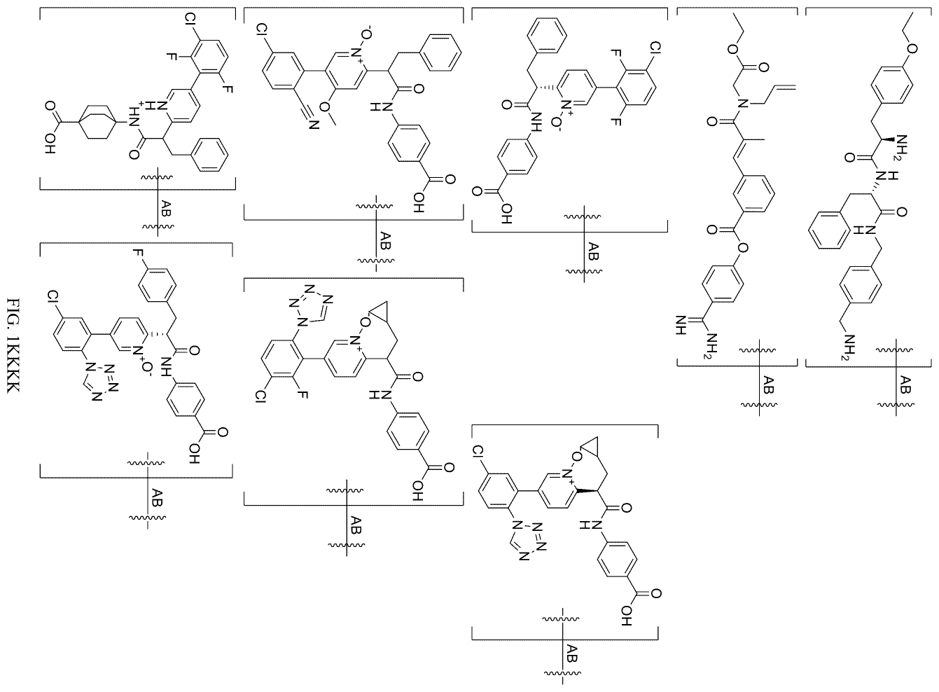

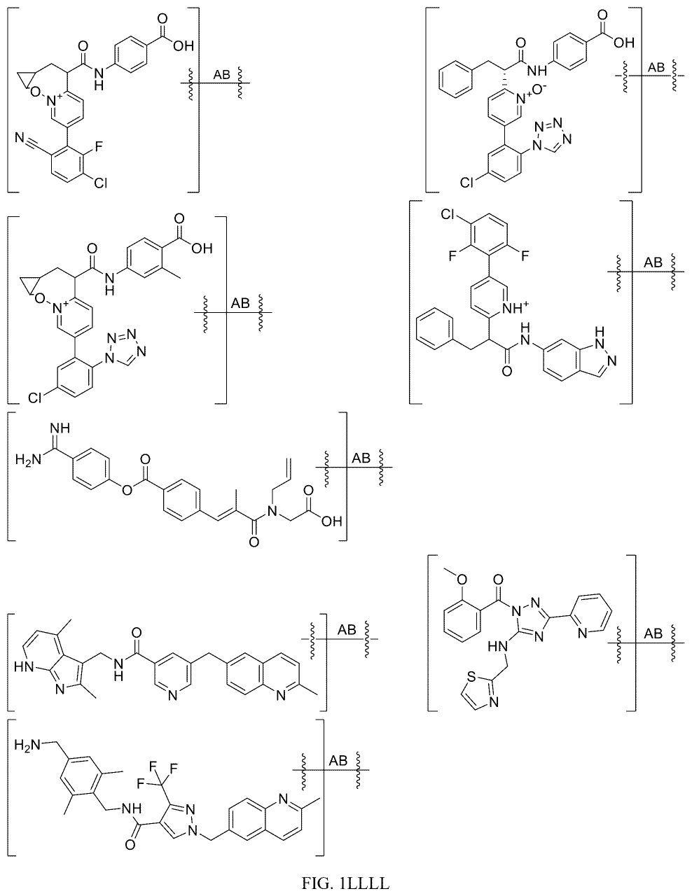

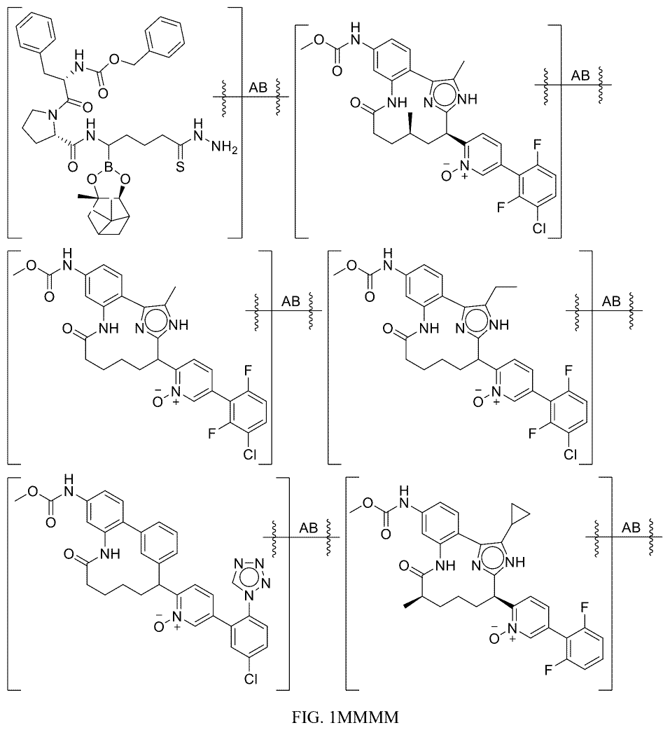

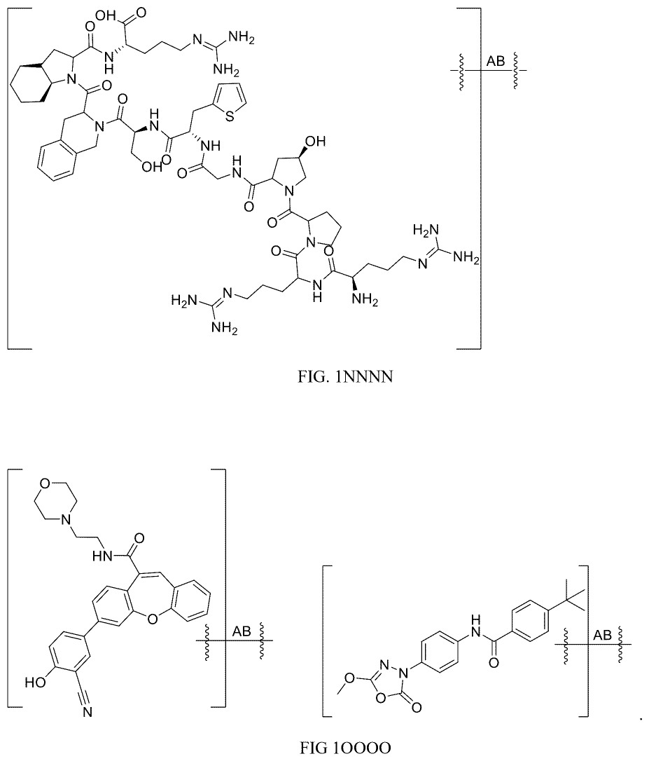

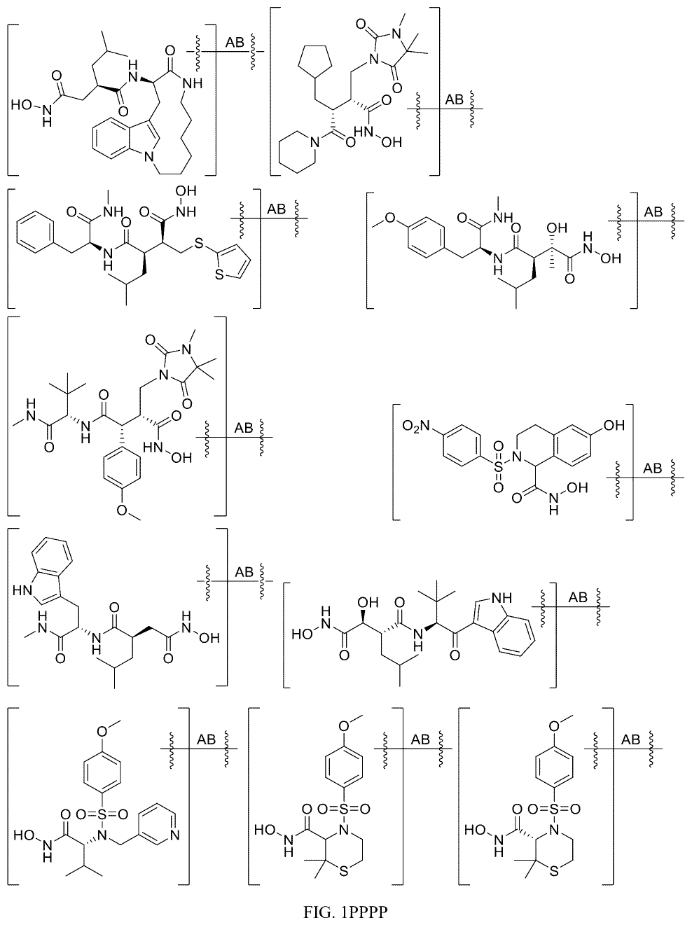

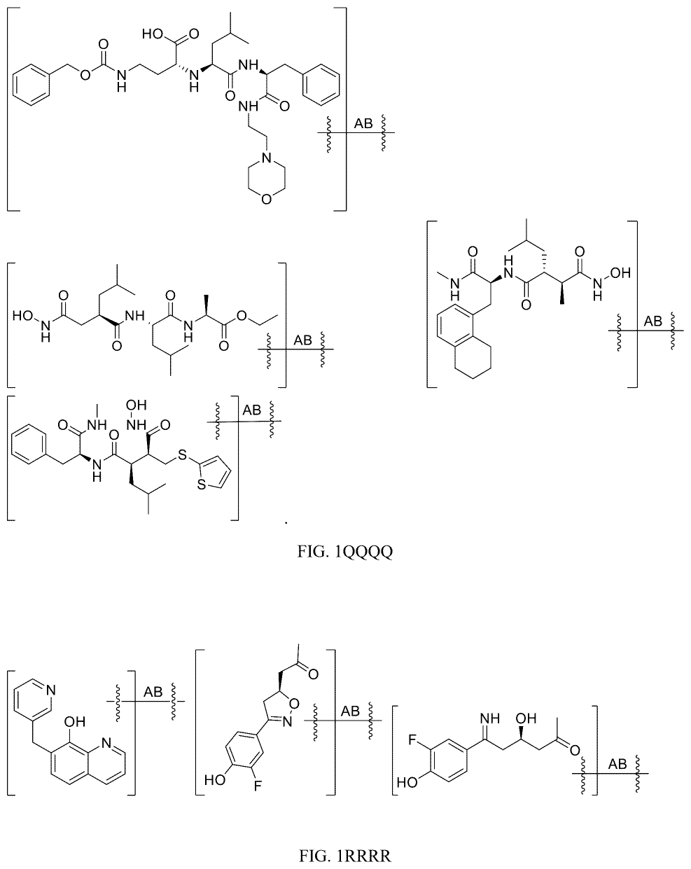

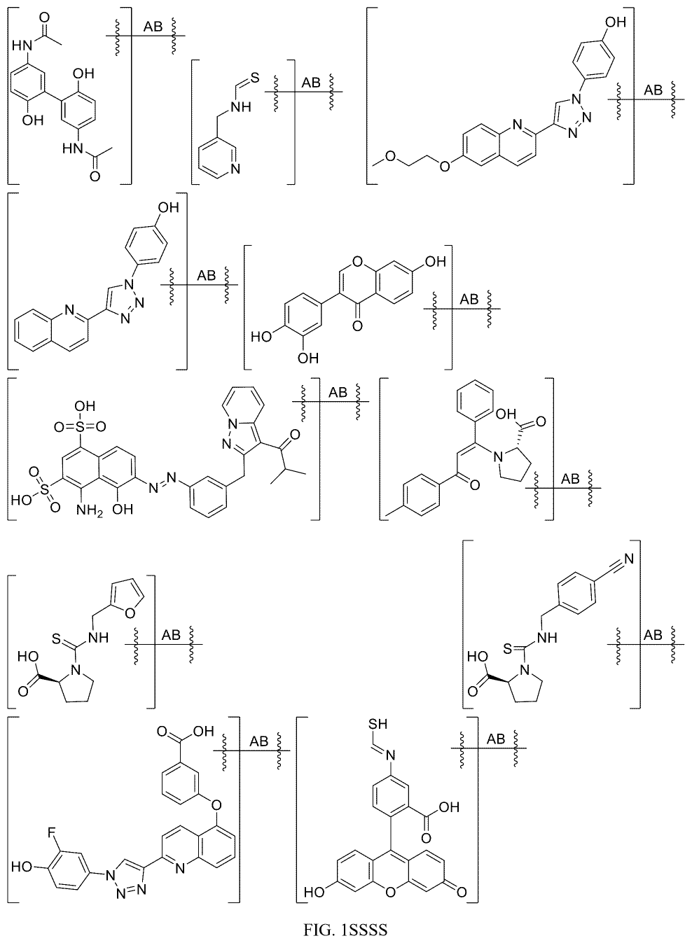

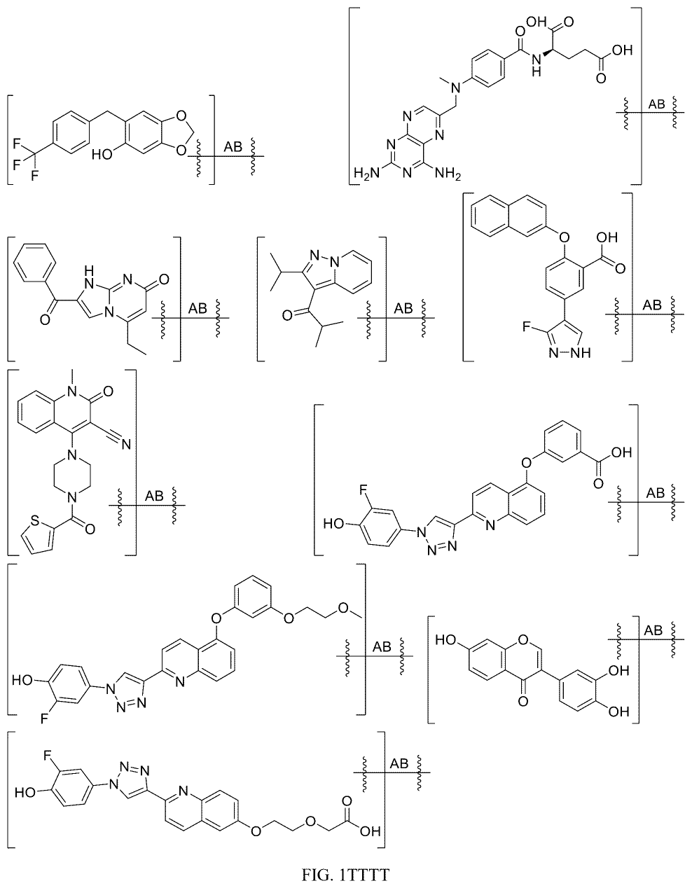

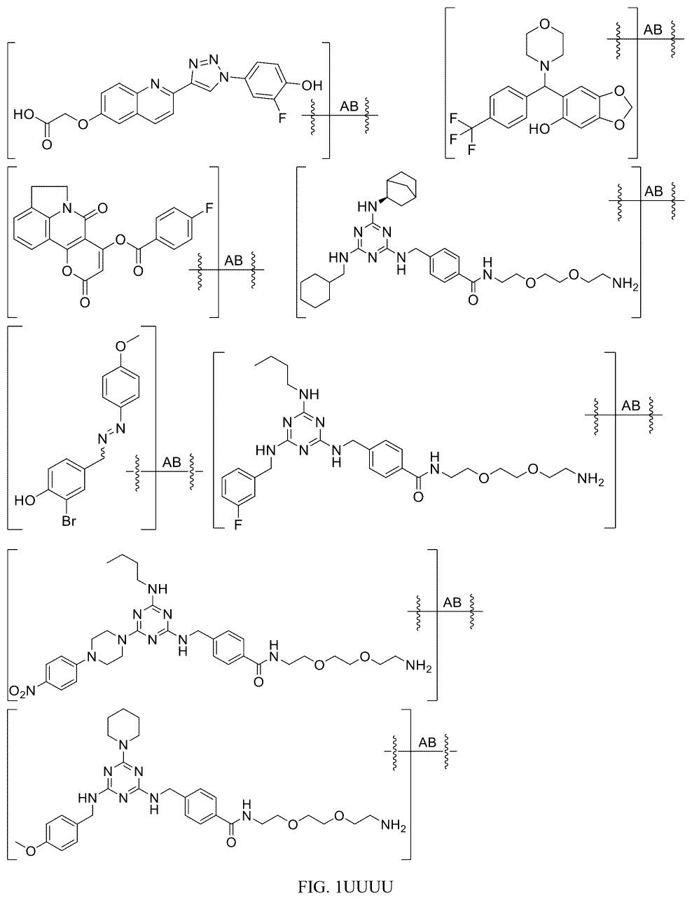

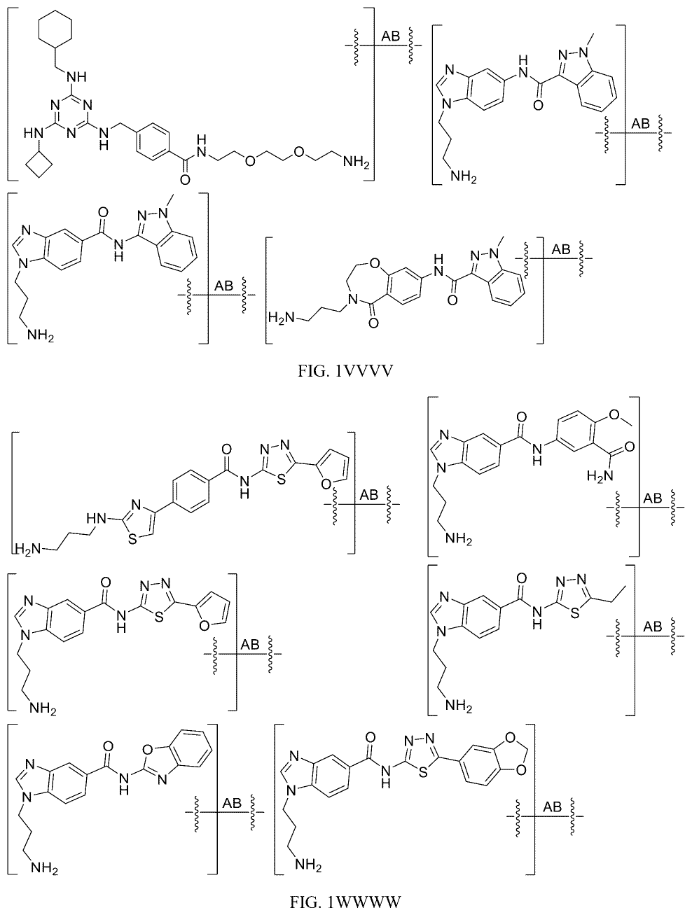

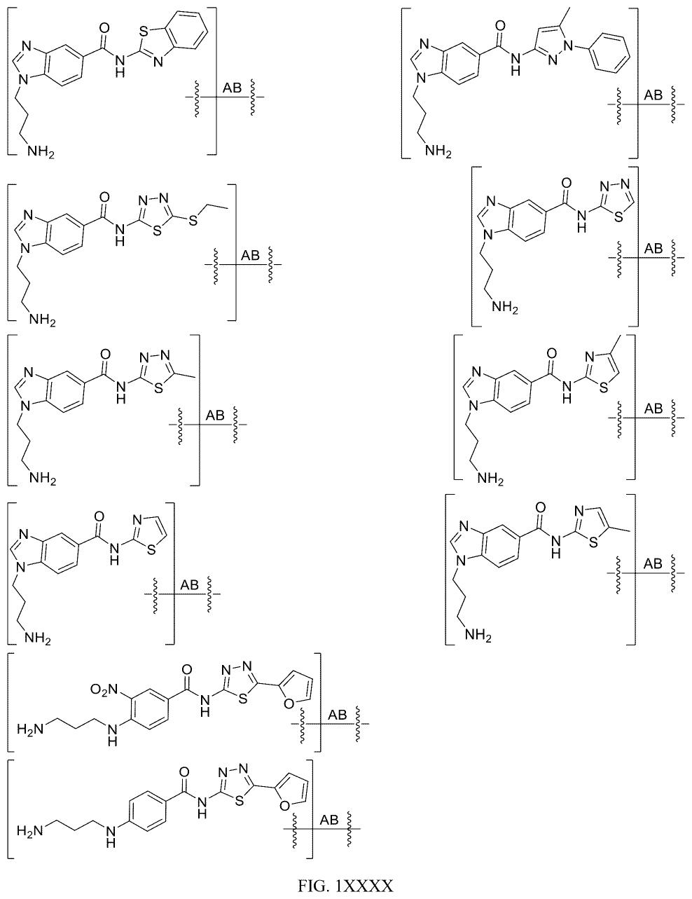

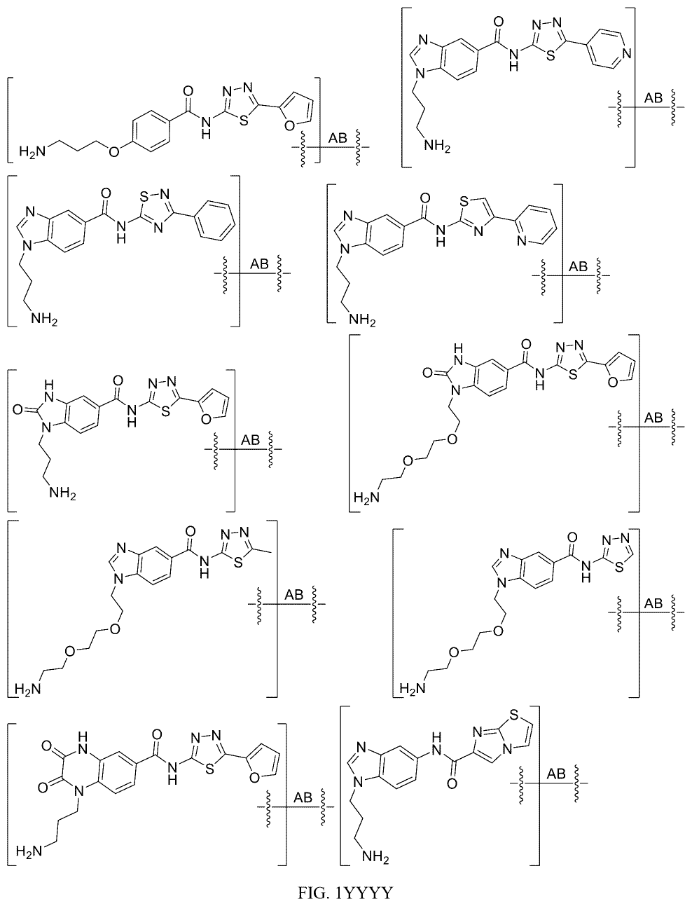

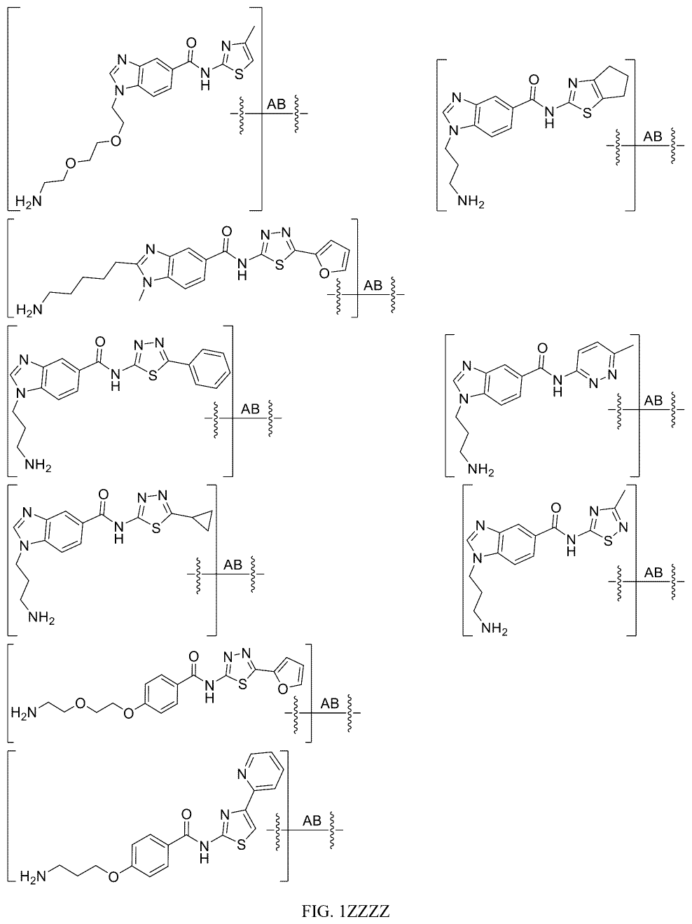

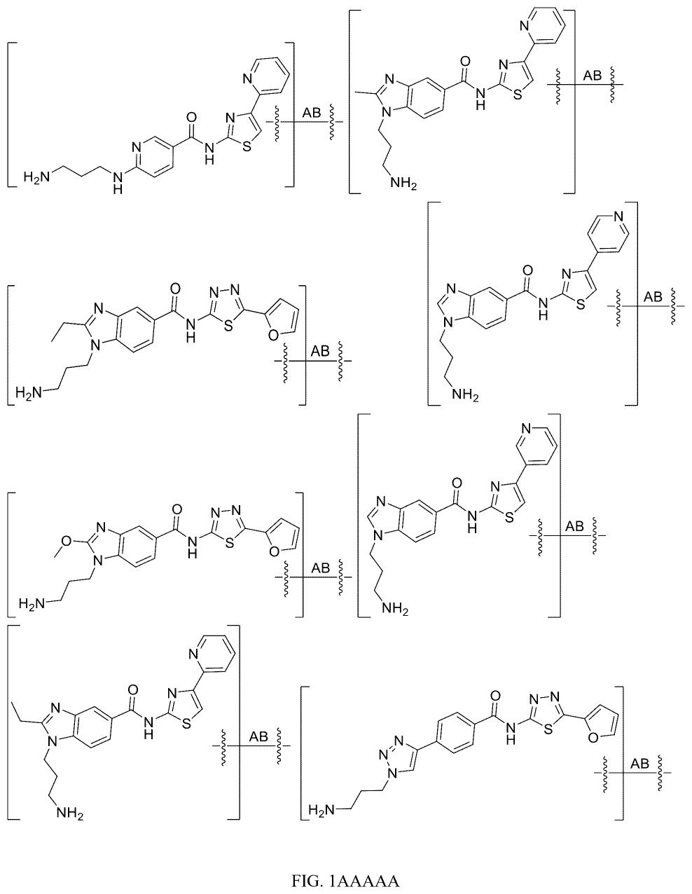

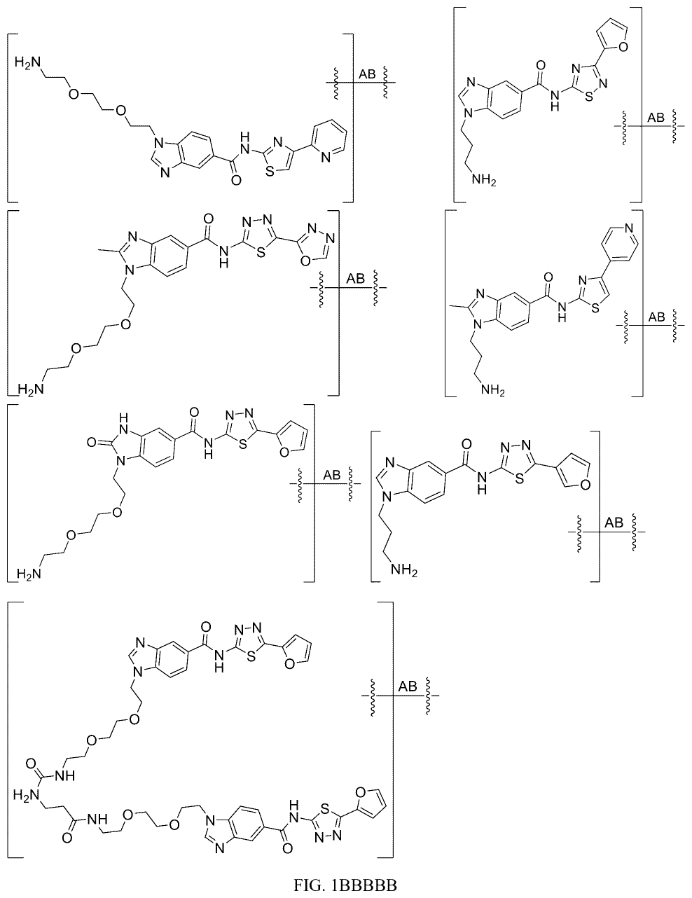

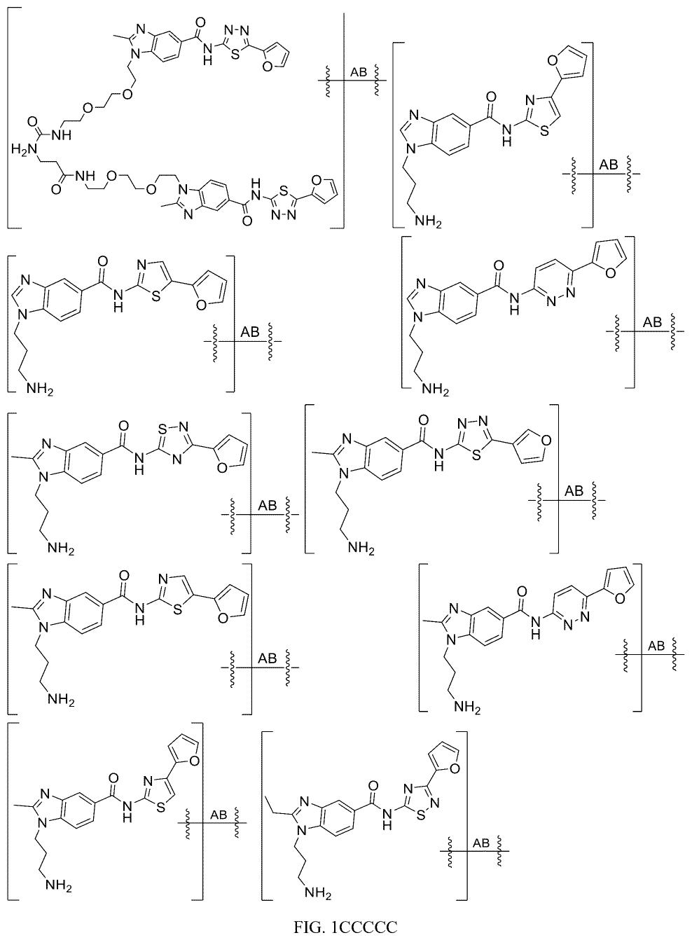

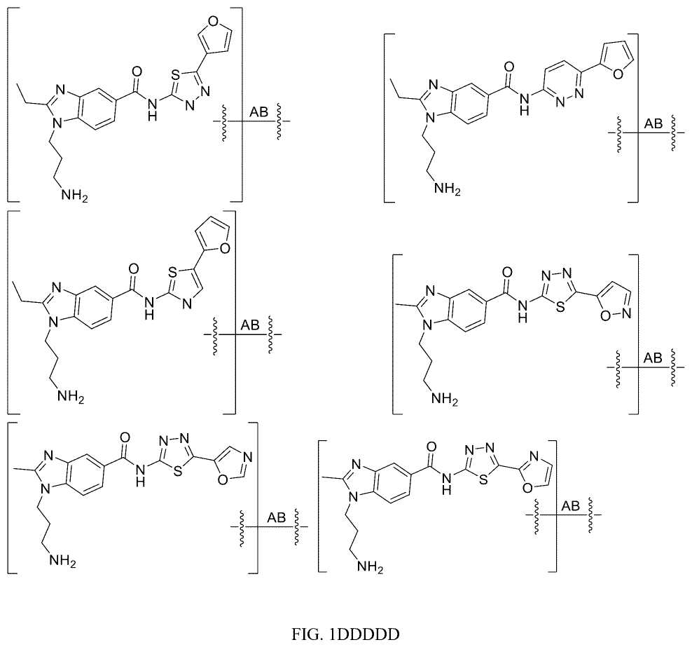

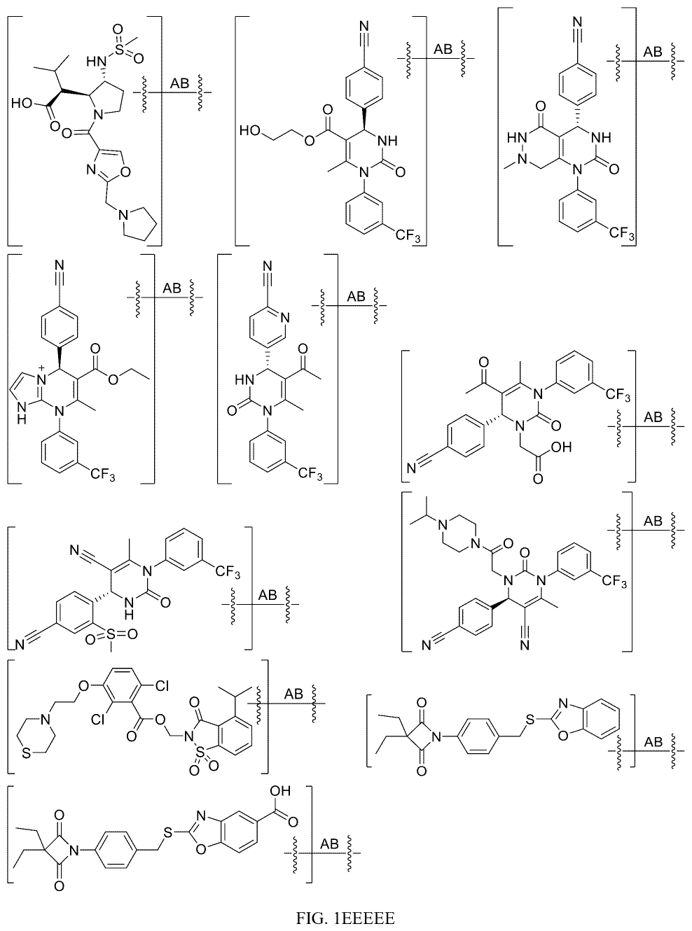

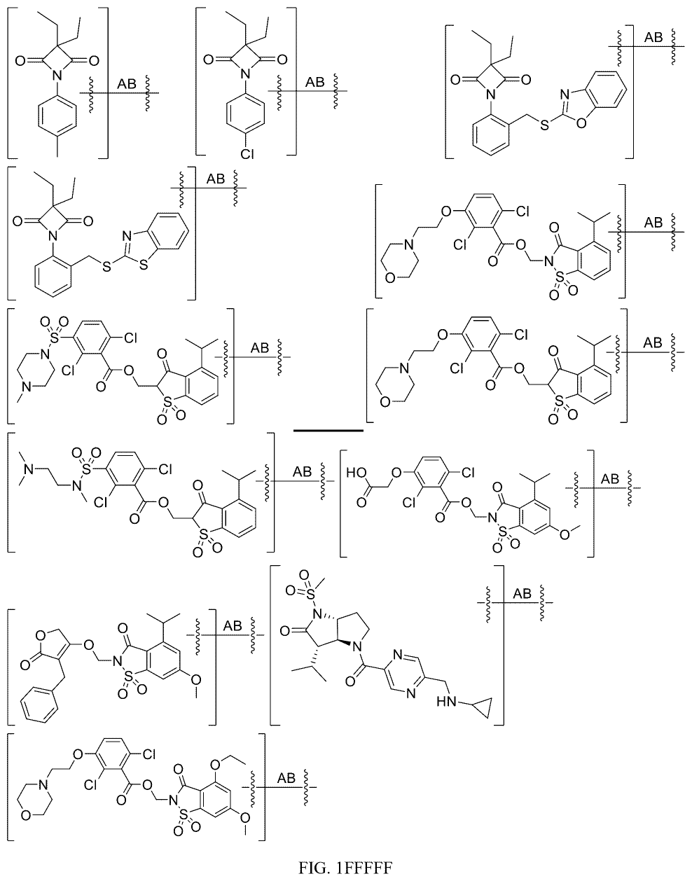

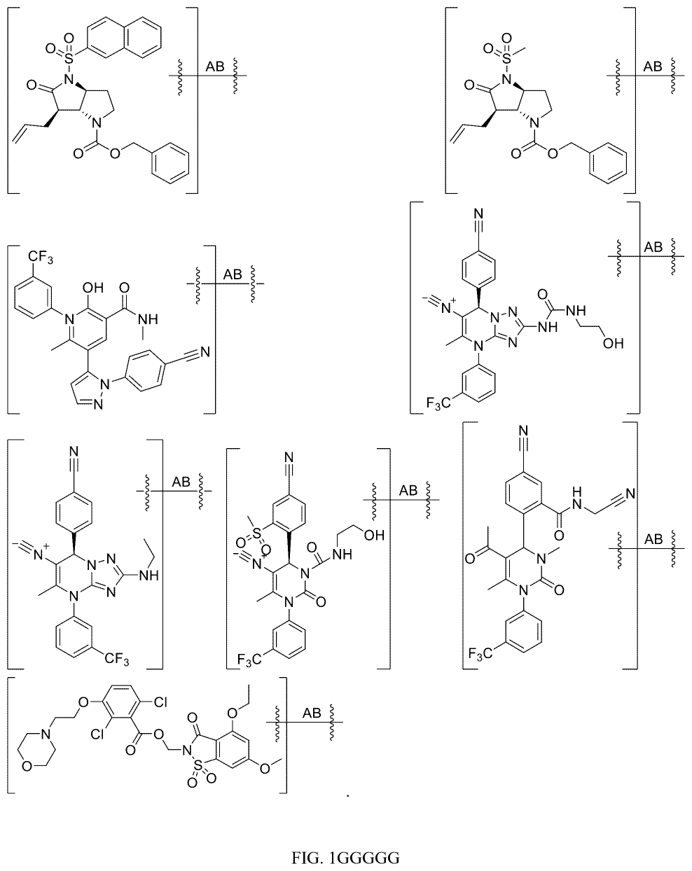

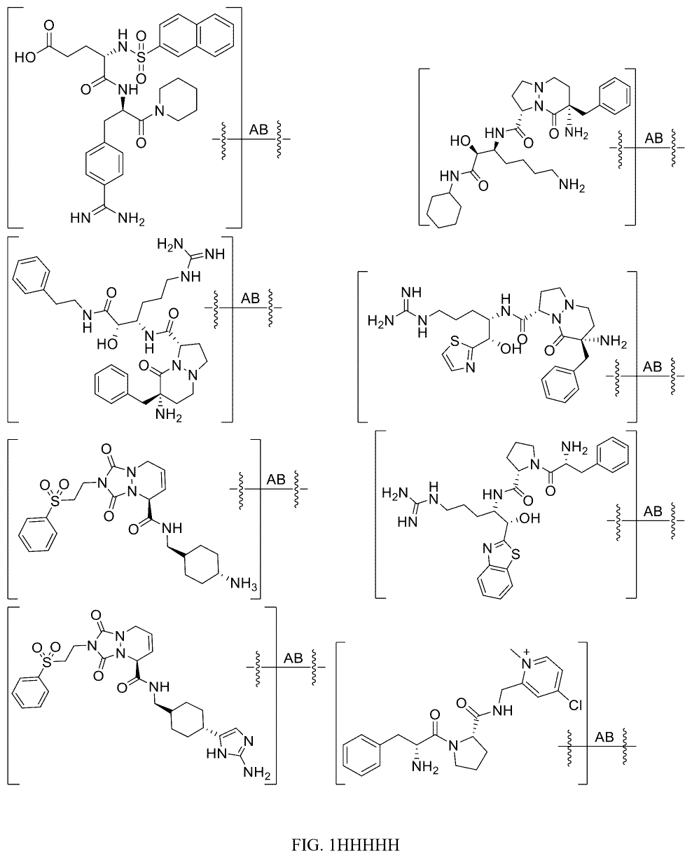

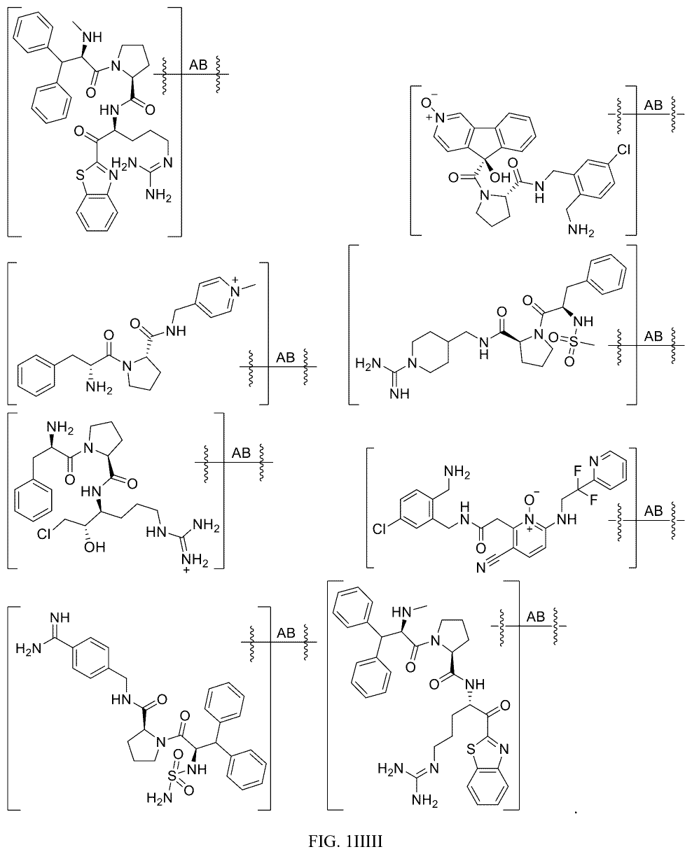

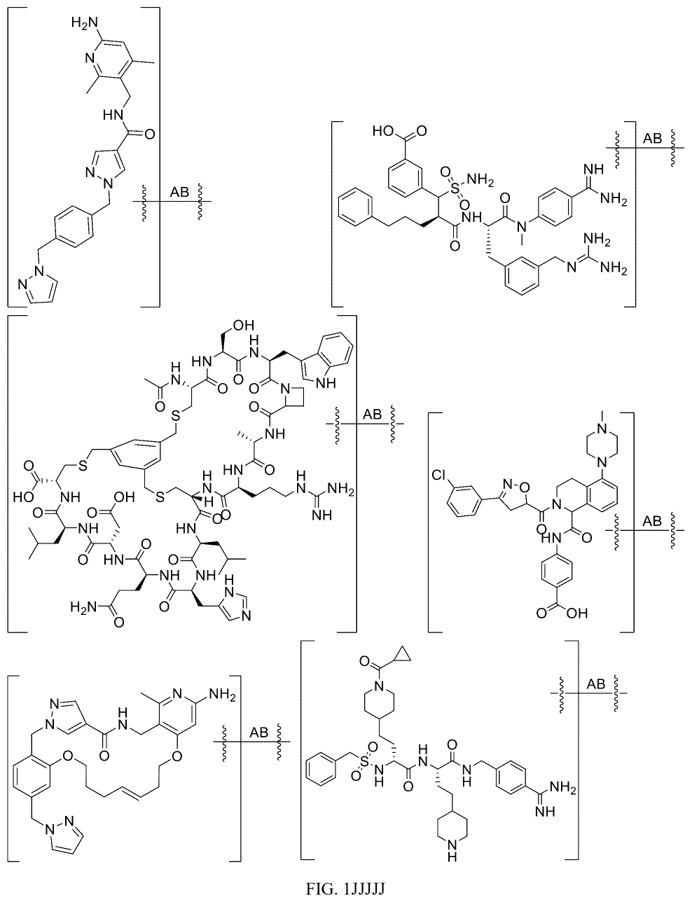

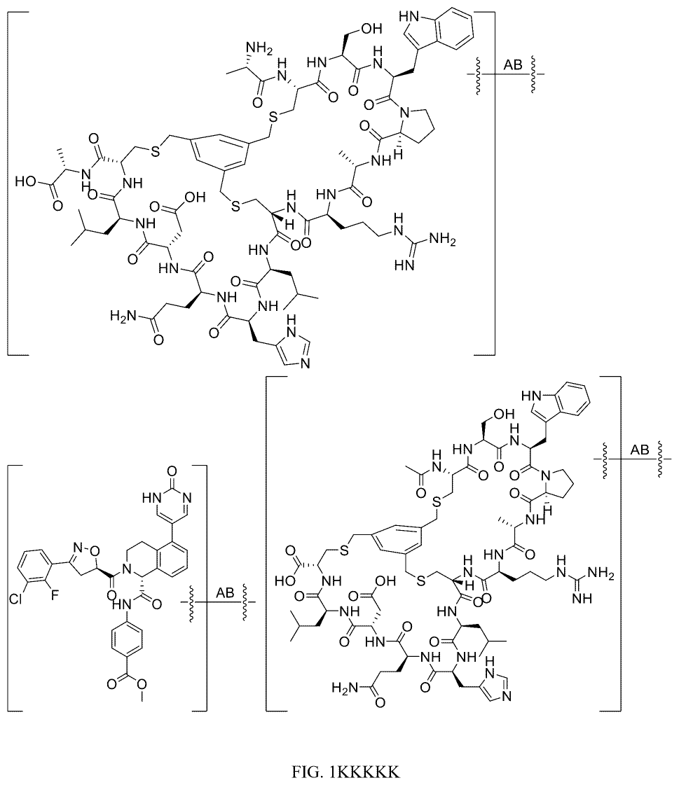

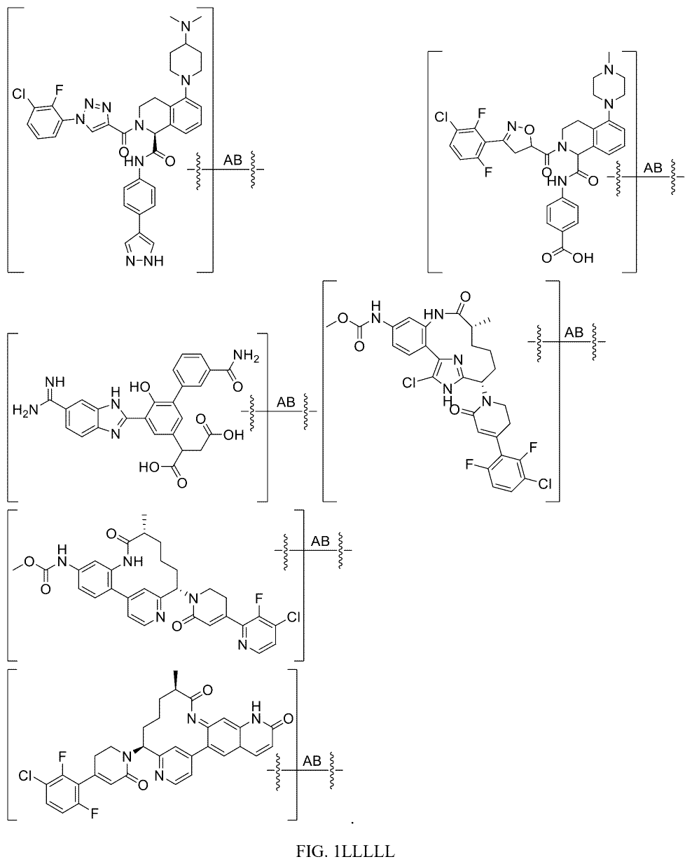

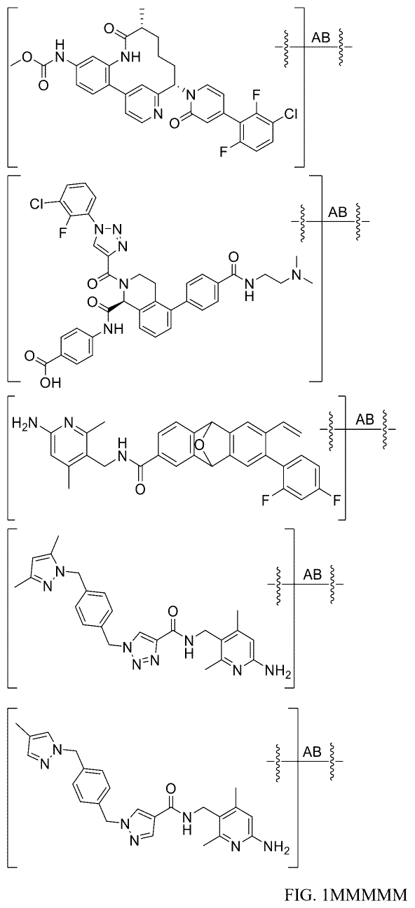

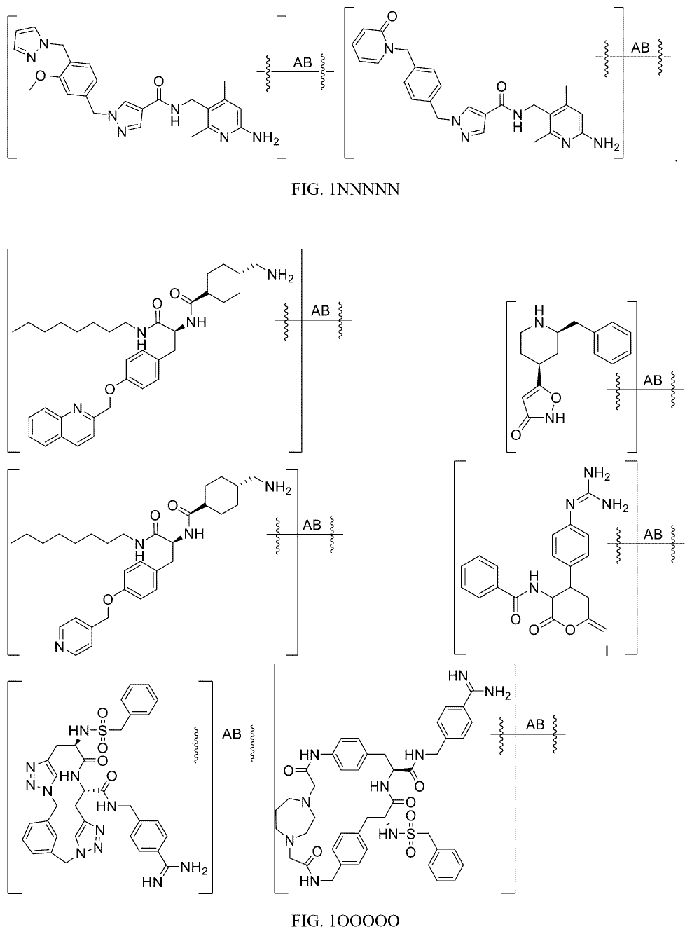

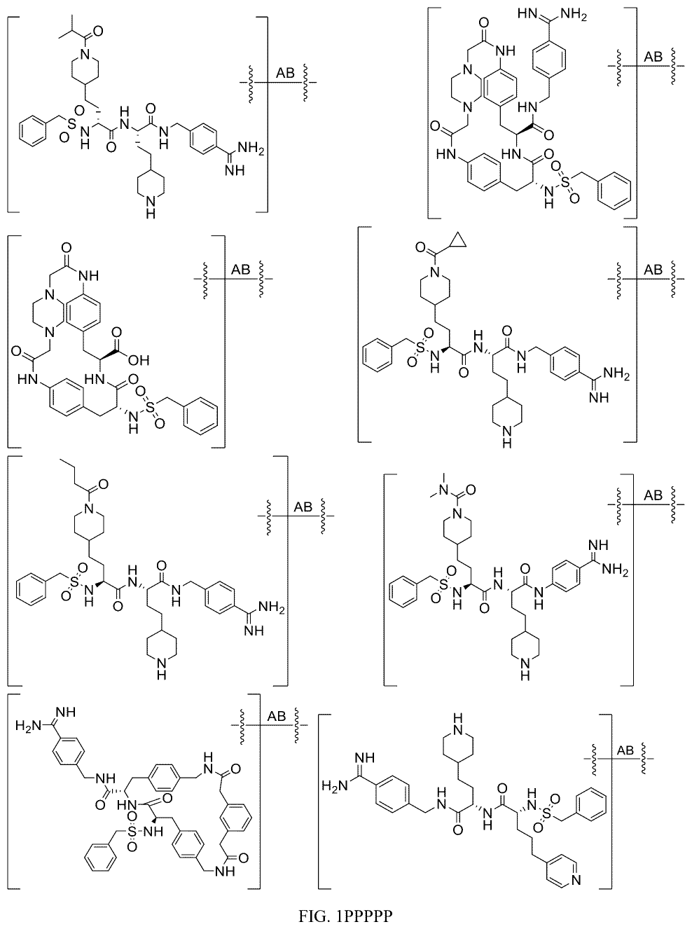

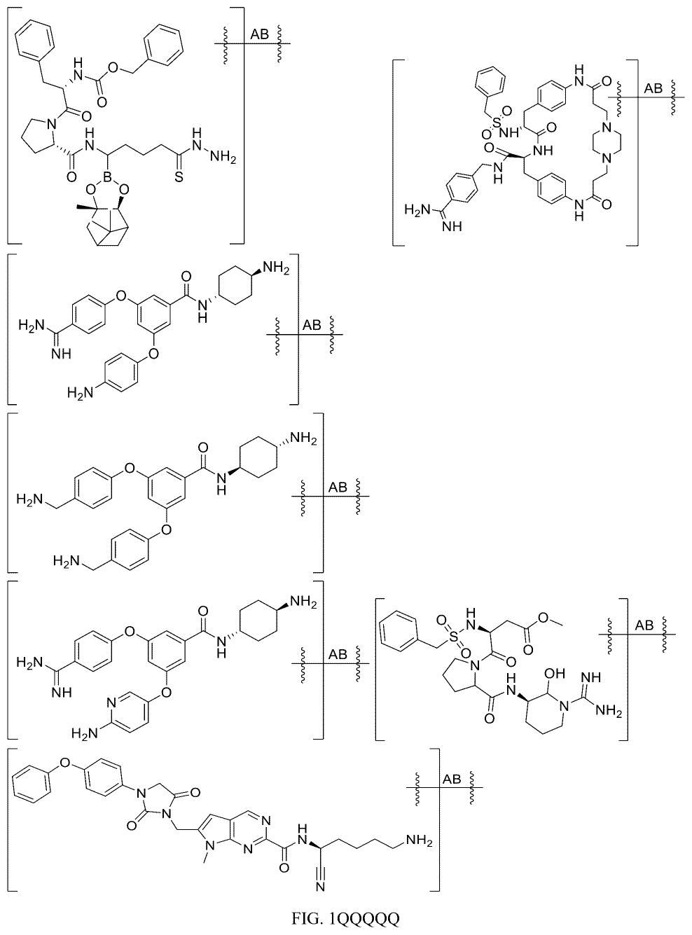

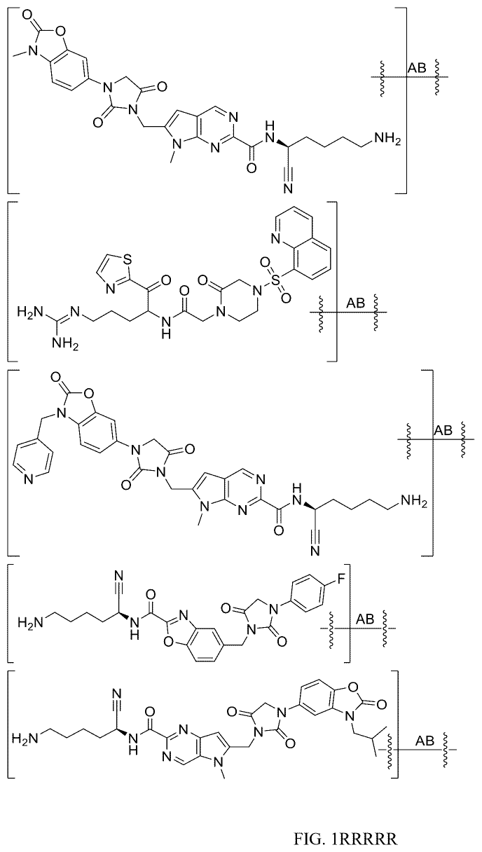

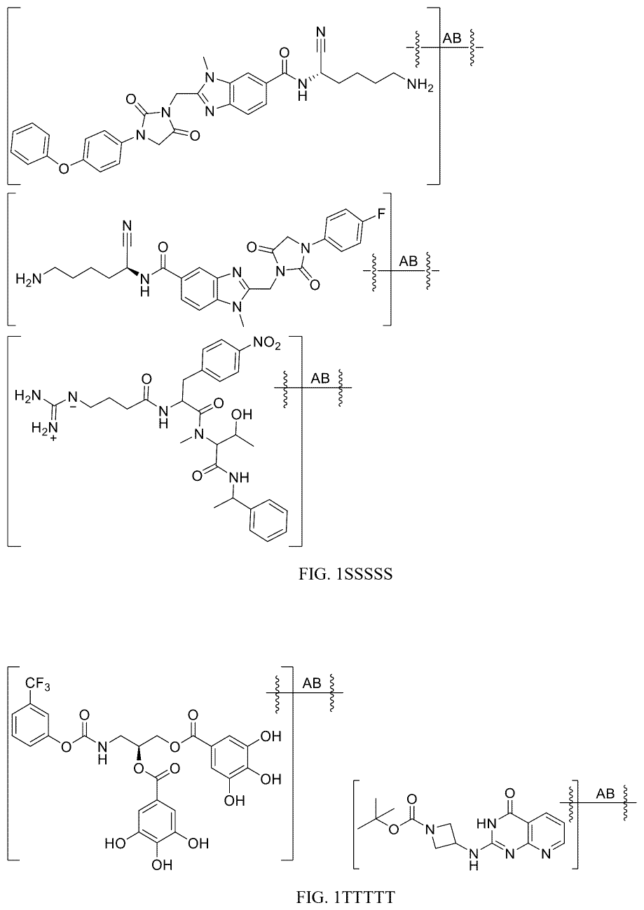

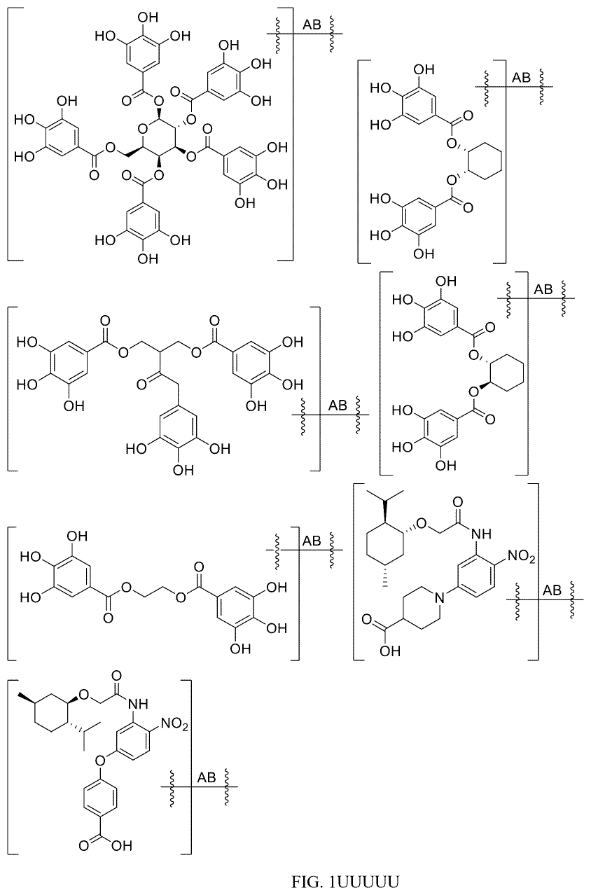

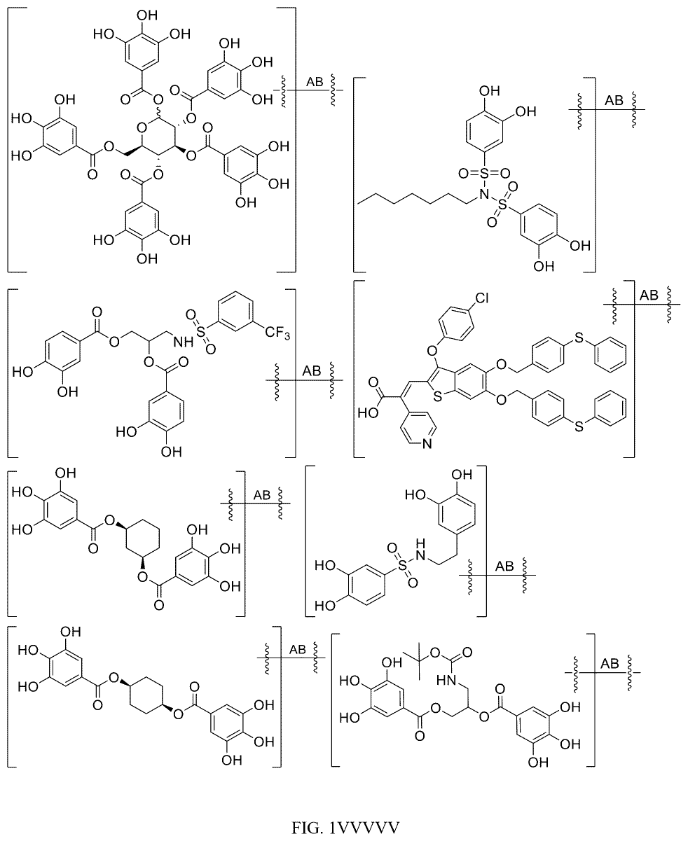

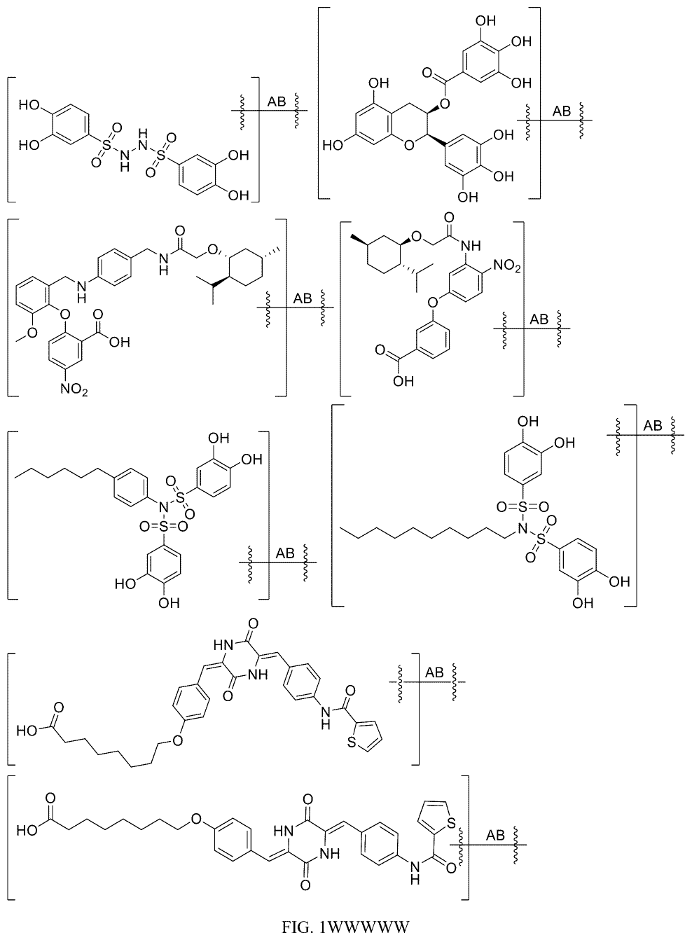

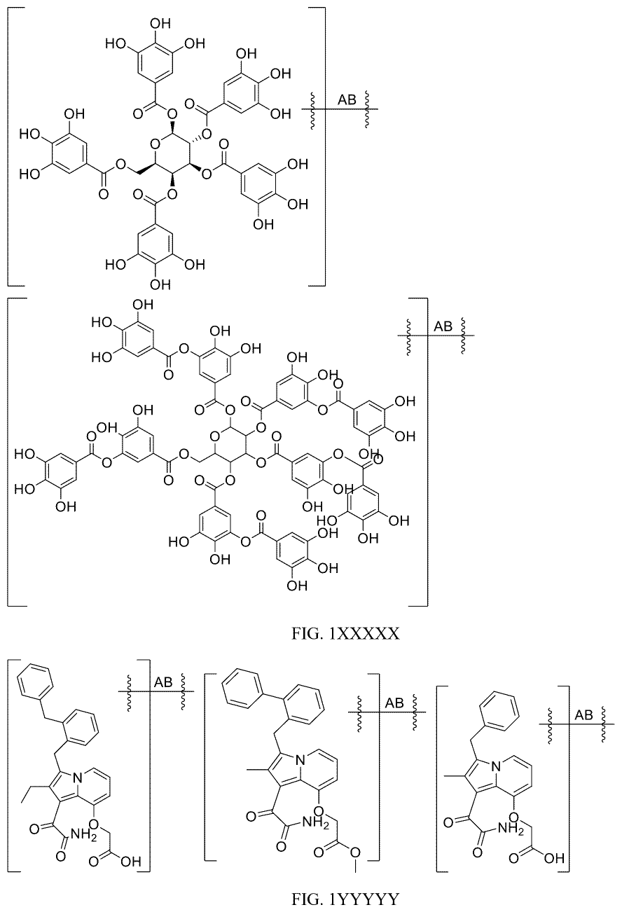

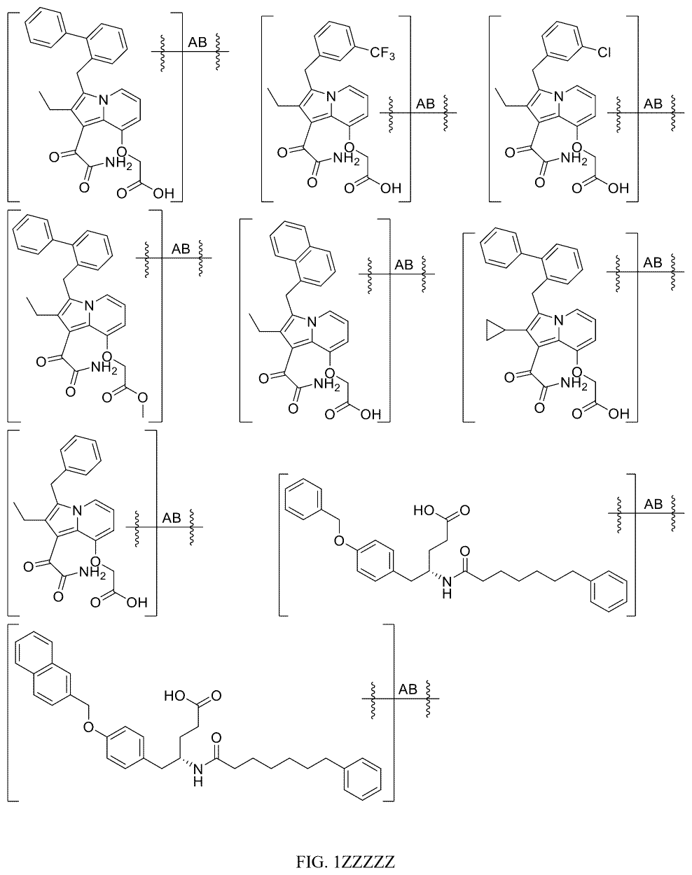

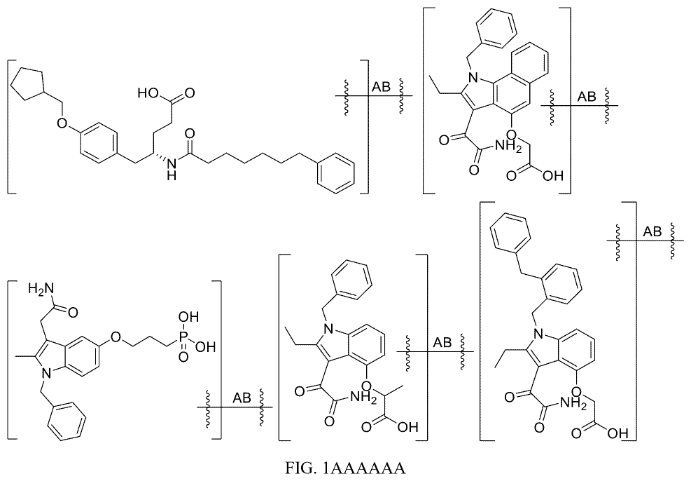

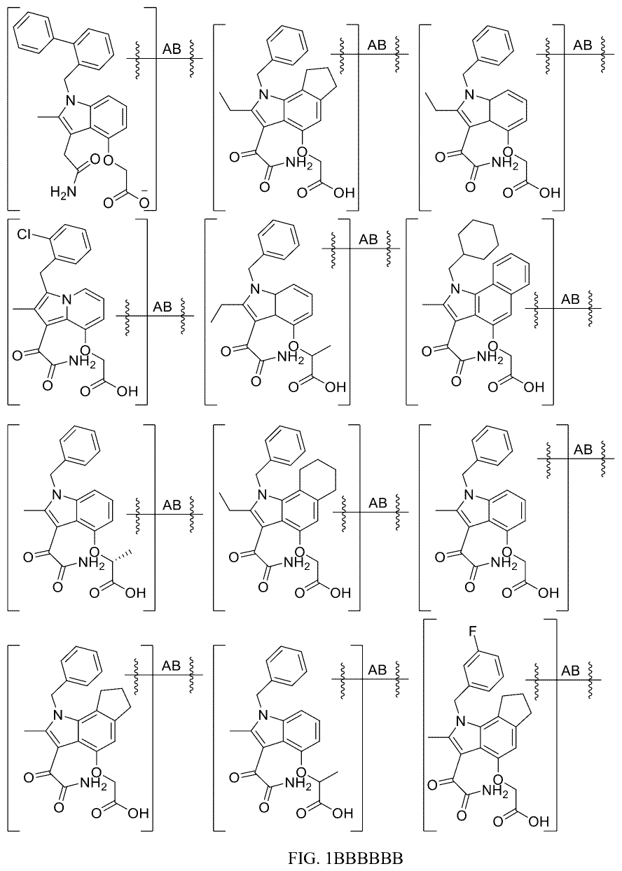

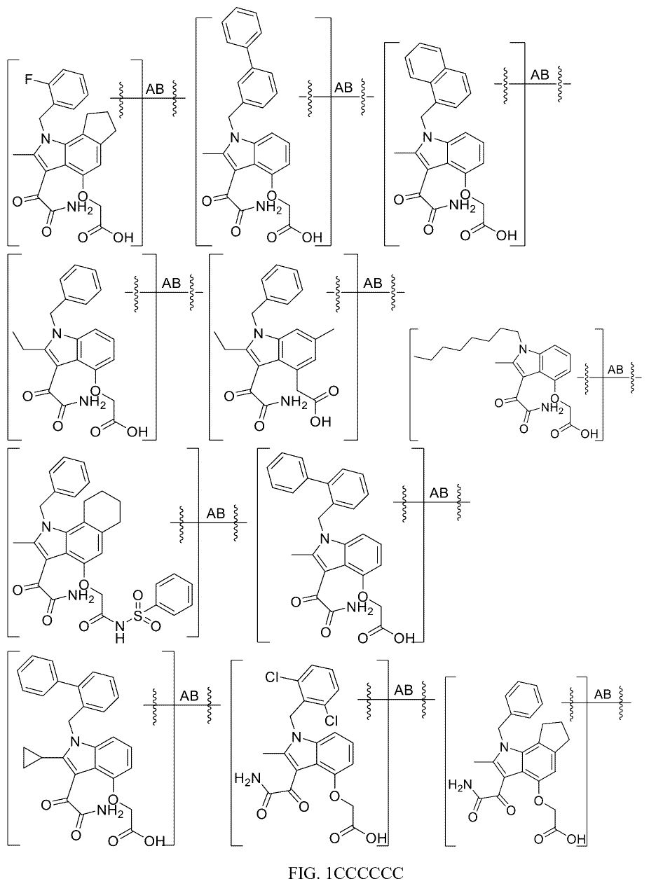

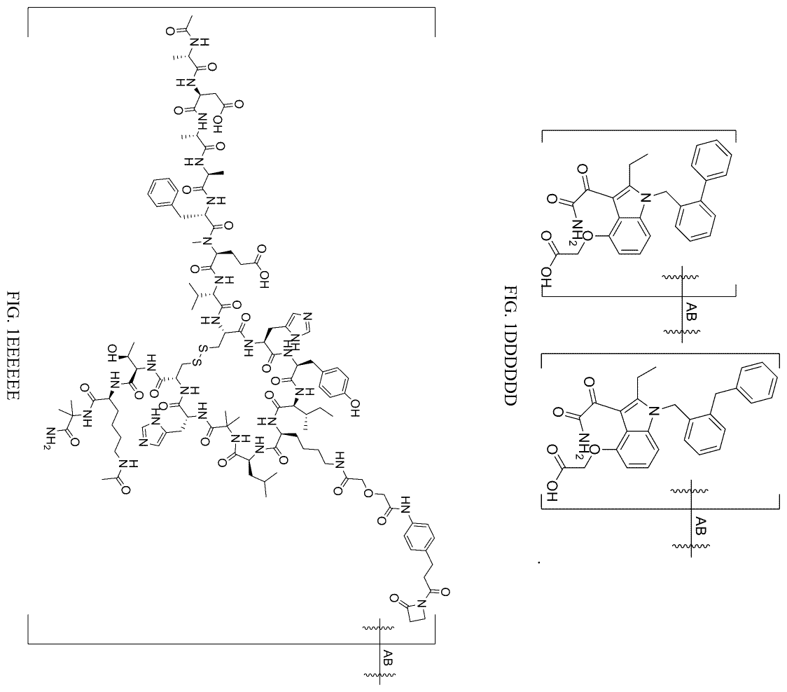

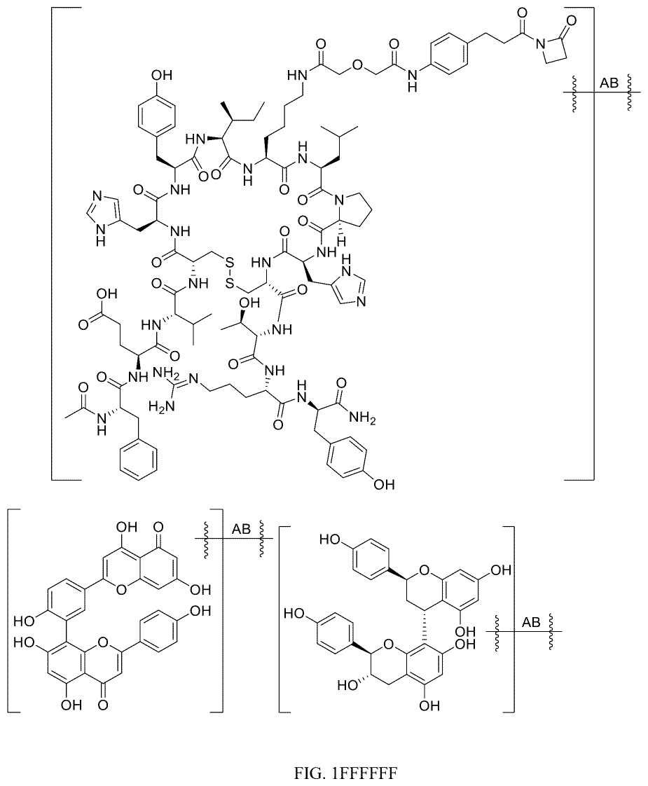

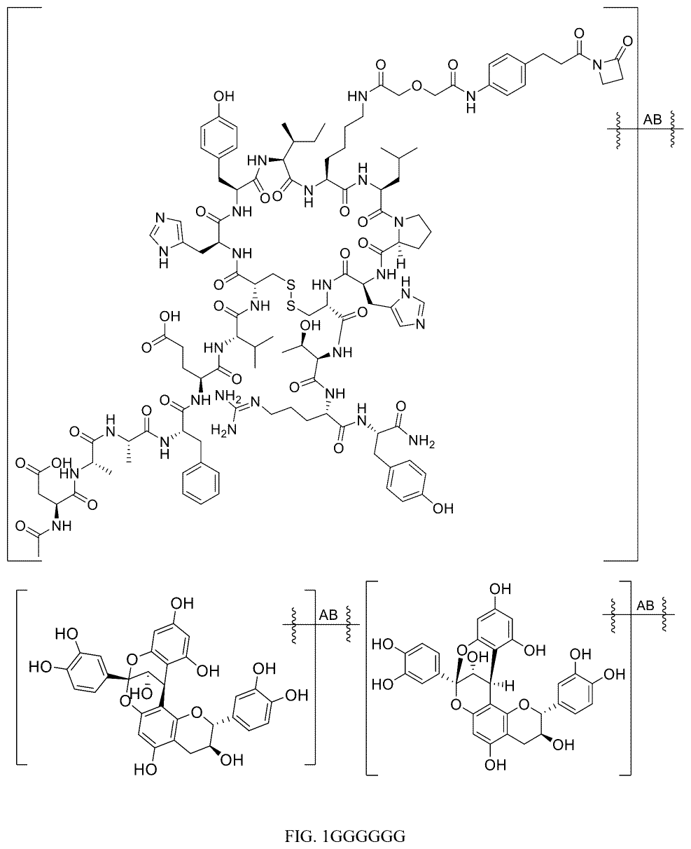

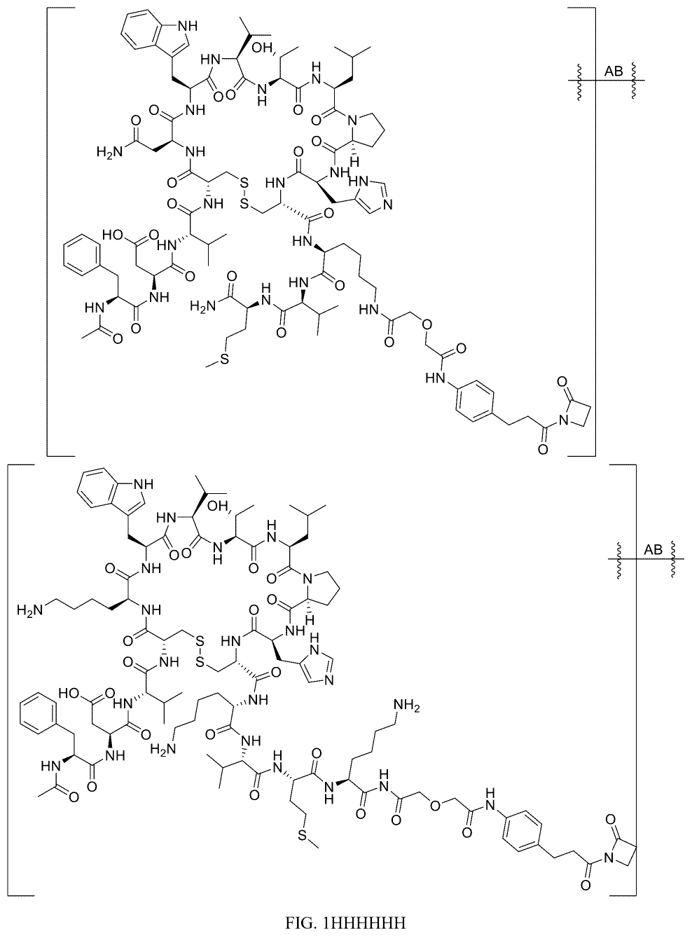

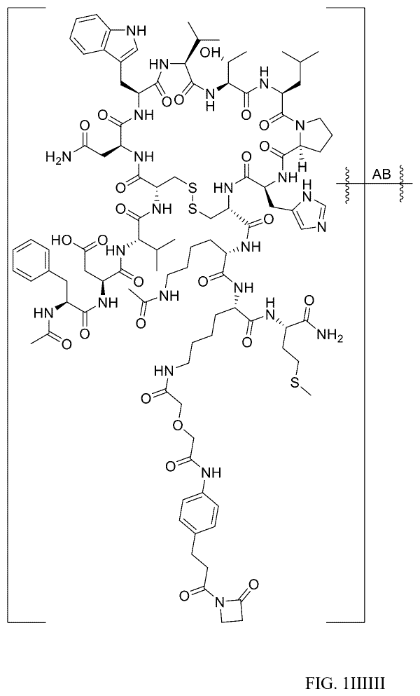

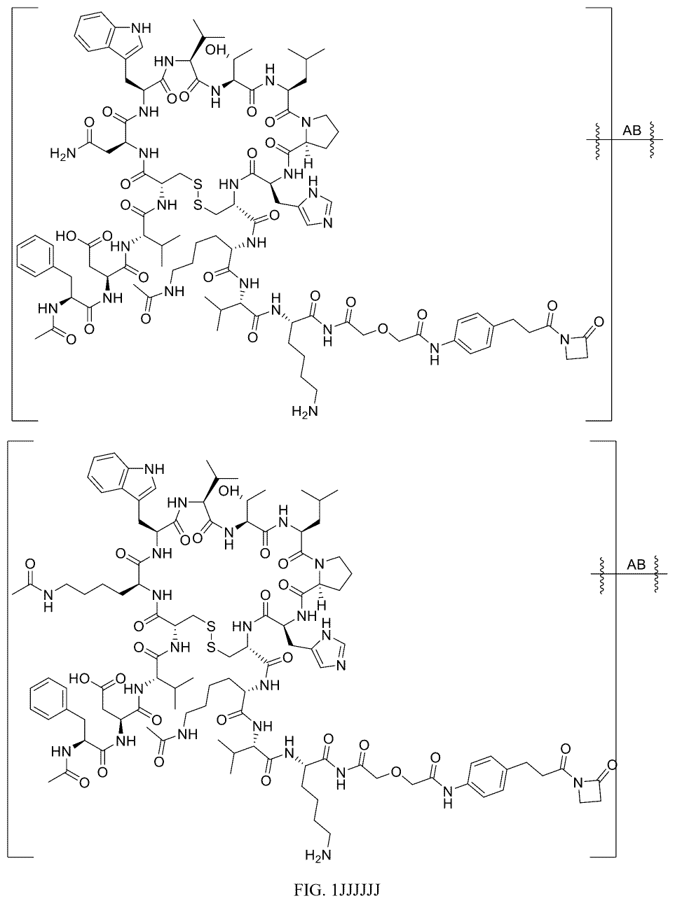

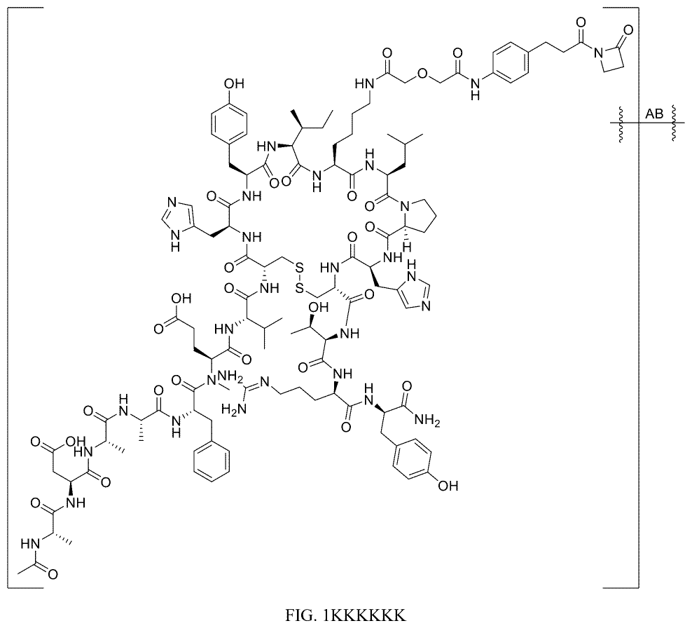

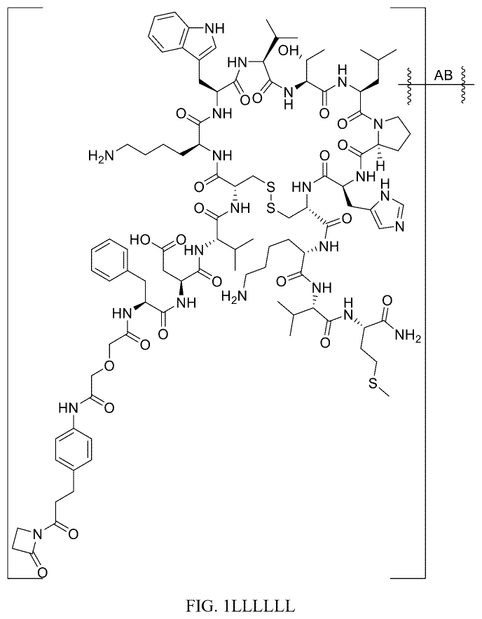

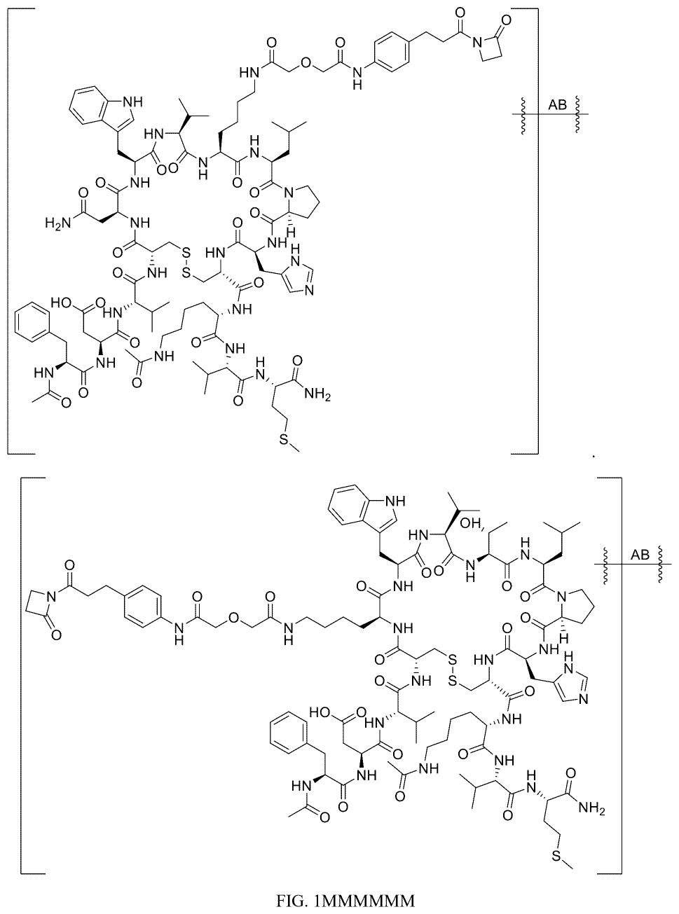

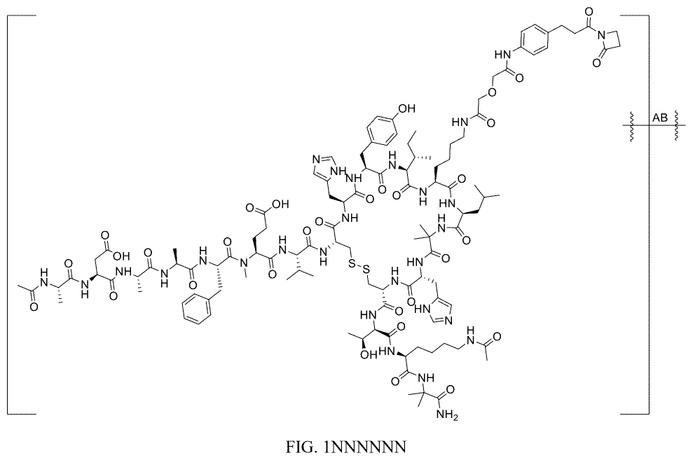

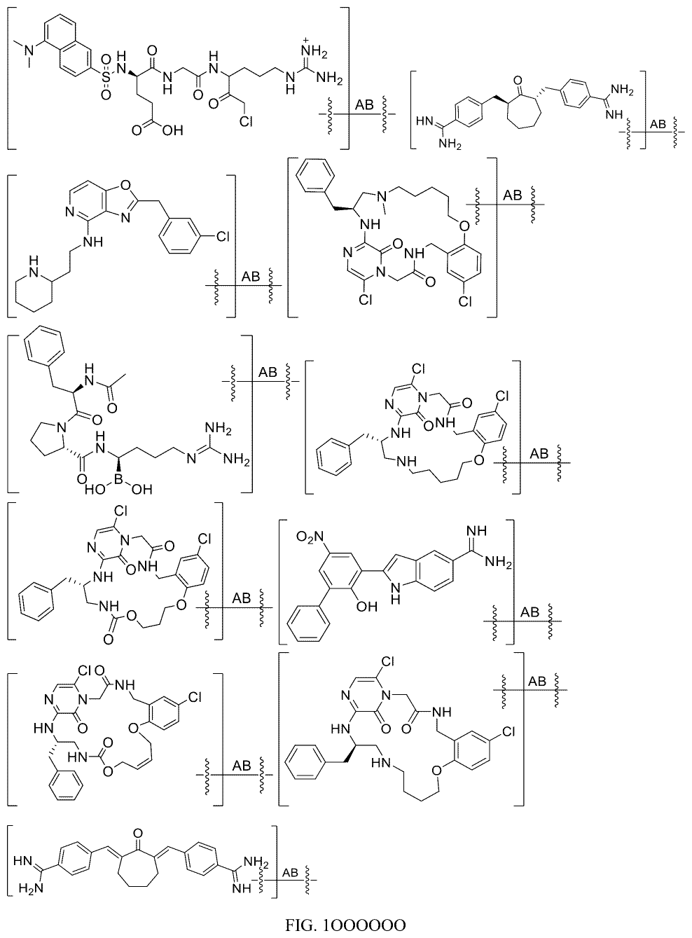

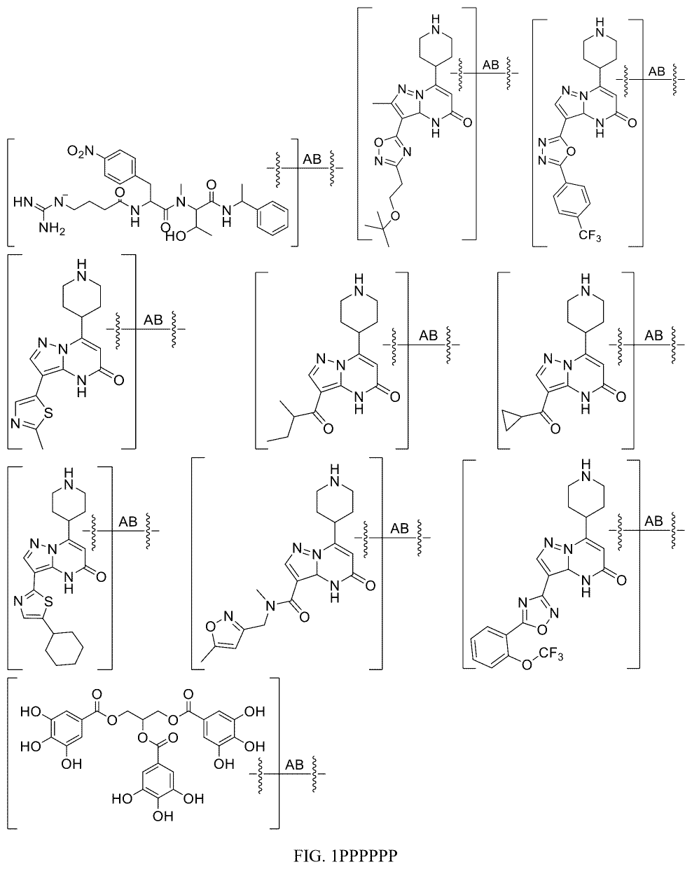

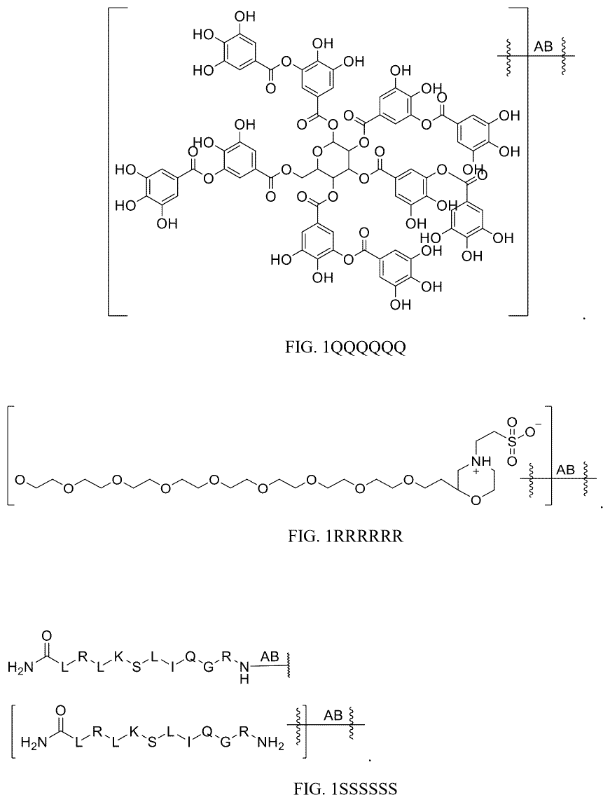

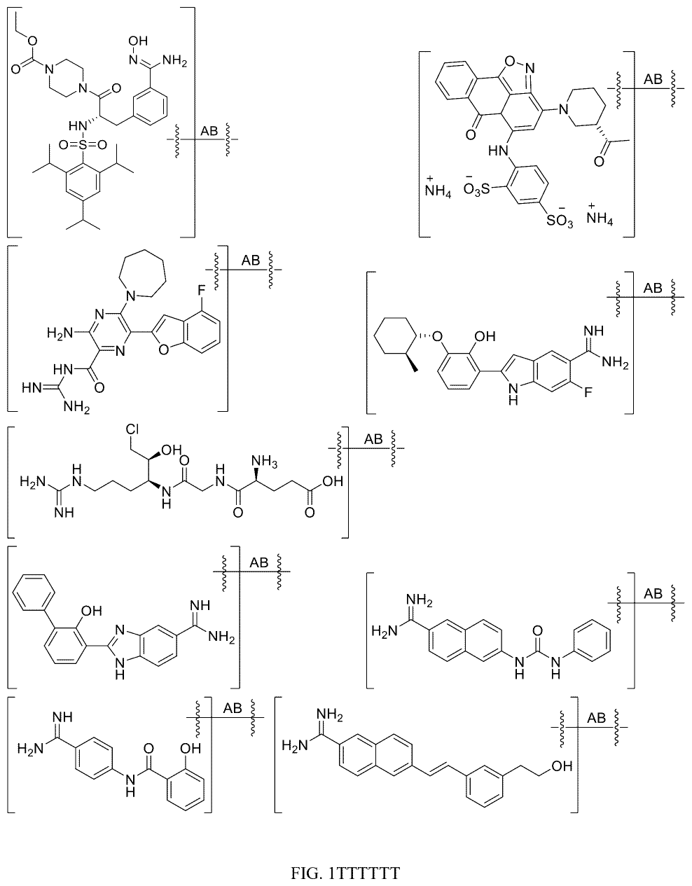

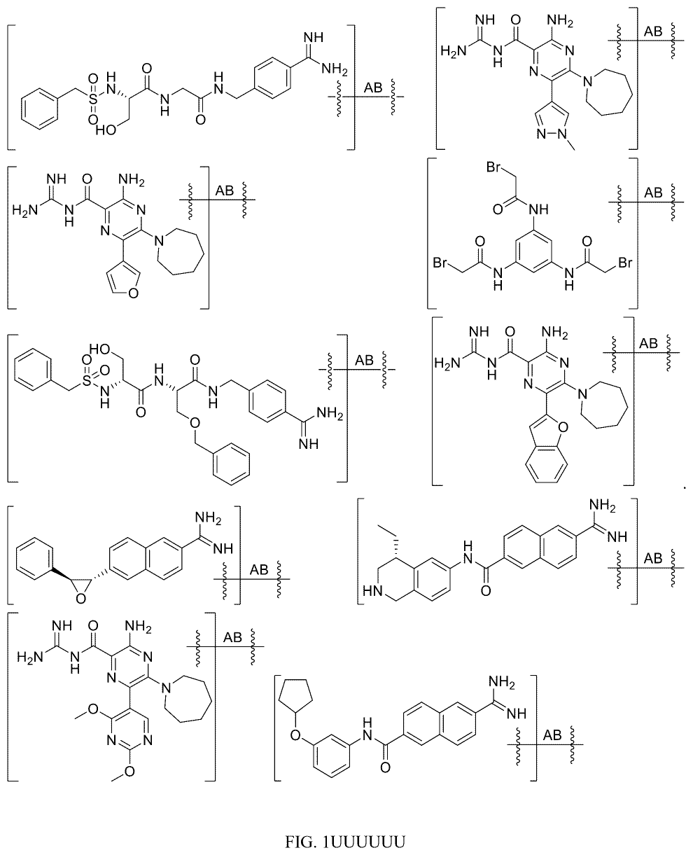

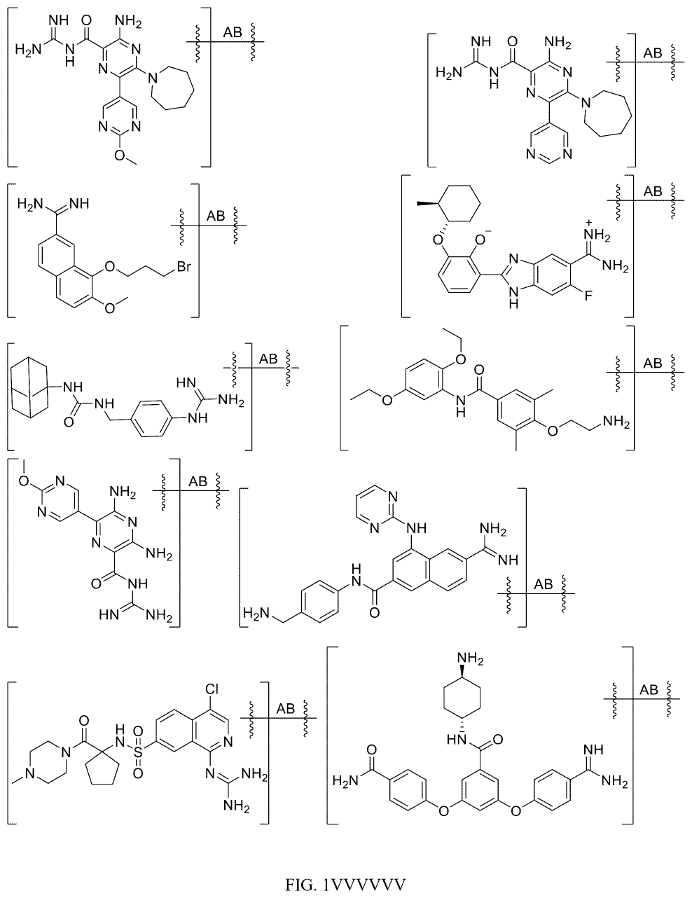

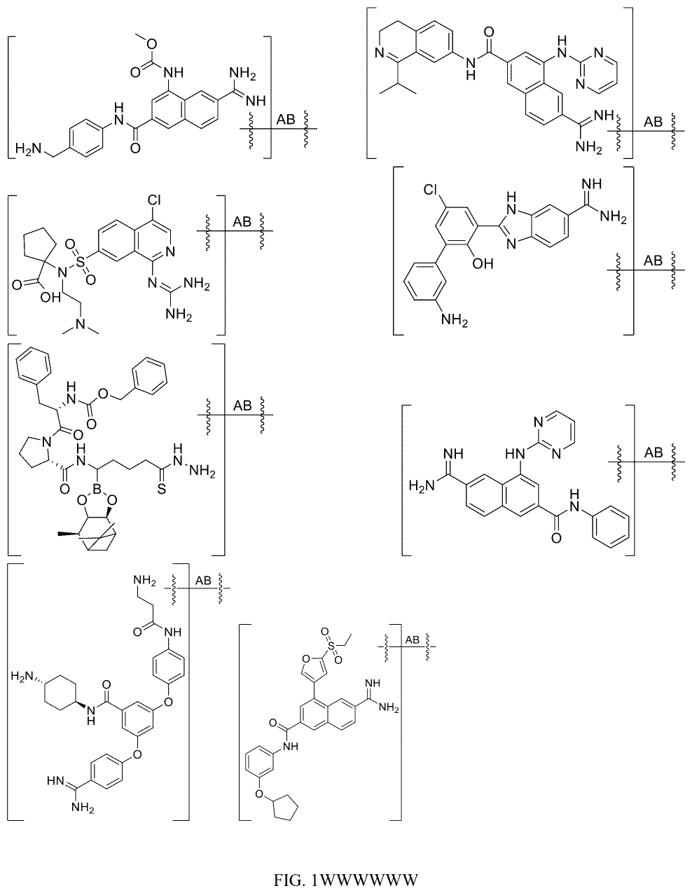

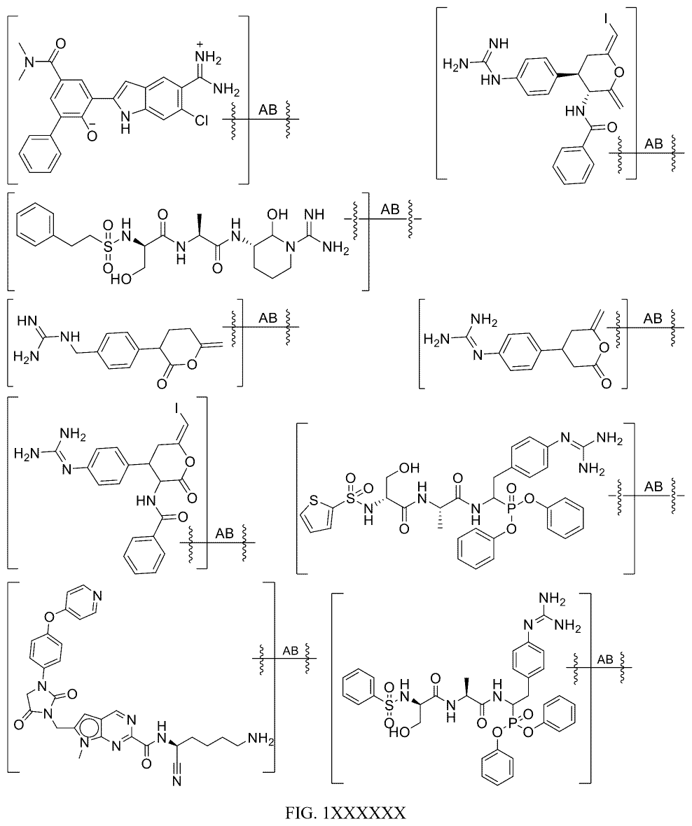

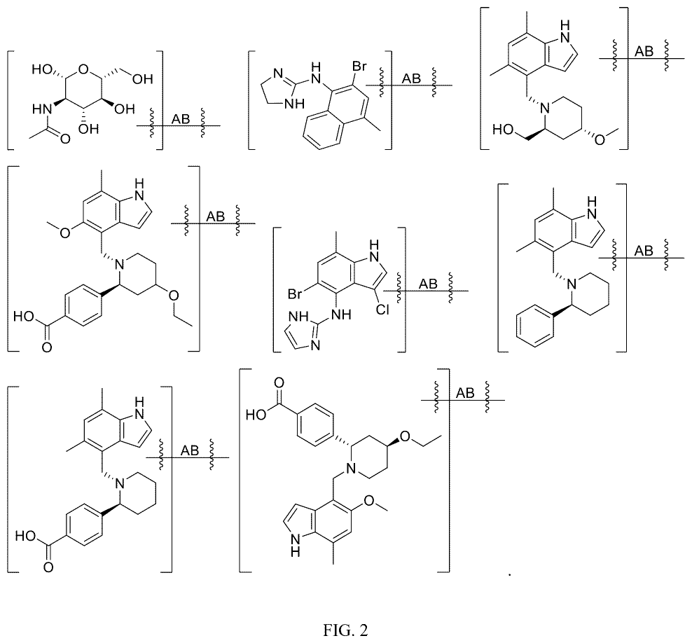

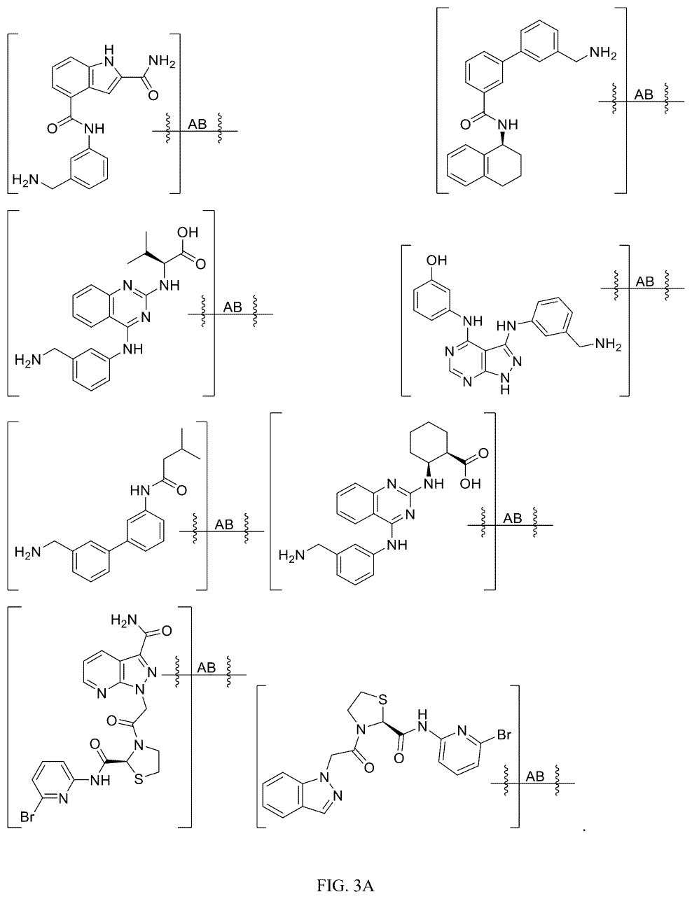

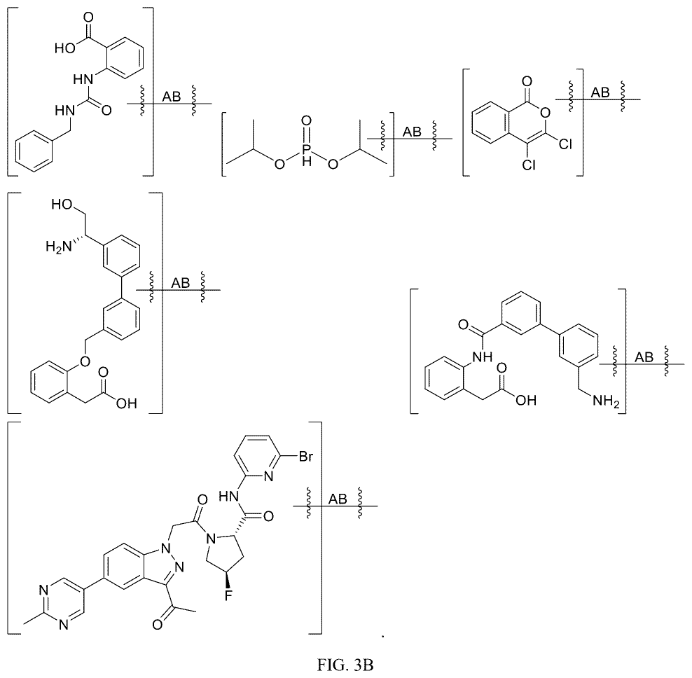

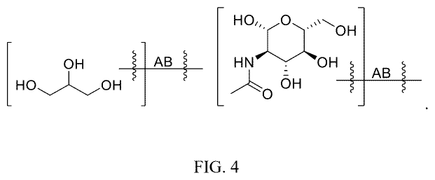

Potential IL-1 direct or indirect inhibitors are described in FIG. 1. Additional IL-1 Targeting Ligands can be found in, for example, U.S. Pat. No. 9,694,015, each of which is incorporated herein by reference. Additional binding ligands include rilanocept or a binding fragment thereof (J Rheumatol. 2012; 39:720-727 (2012); and Canakinumab, or a binding fragment thereof (J Rheumatol. 2004; 31:1103-1111).

In certain embodiments the IL-1 Targeting Ligand is selected from

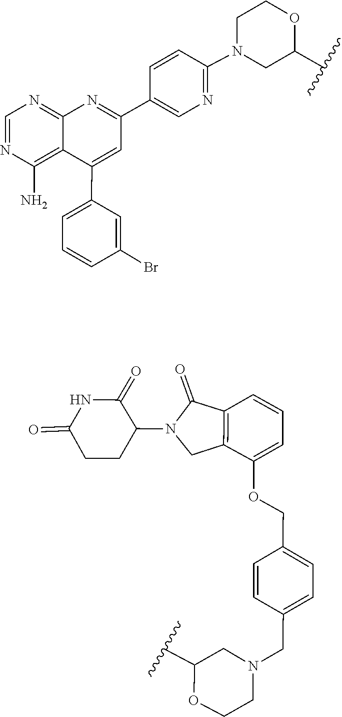 [SMILES: *C1CN(c2ccc(-c3cc(-c4cccc(Br)c4)c4c(N)ncnc4n3)cn2)CCO1 ; *C1CN(Cc2ccc(COc3cccc4c3CN(C3CCC(=O)NC3=O)C4=O)cc2)CCO1]

### IL-2

In some embodiments, the Target Extracellular Protein is human interleukin-2 (IL-2) (UniProtKB—P60568 (IL2_HUMAN)). IL-2 is a potent pro-inflammatory cytokine. IL-2 has been implicated in host versus graft rejection and other autoimmune disorders.

The Protein Data Bank website provides the crystal structure of IL-2 searchable by 1M4C and 1M47 (Arkin, M. R., et al., Proc. Natl. Acad. Sci. USA, 2003, 100: 1603-1608); as well as the crystal structure of IL-2 bound to various compounds searchable by 4NEJ and 4NEM (Brenke, R., et al.); 1QVN (Thanos, C. D., et al., Proc Natl Acad Sci USA, 2006, 103 15422-15427); 1PW6 and 1PY2 (Thanos, C. D., et al., J Am Chem Soc., 2003, 125 15280-15281); 1NBP (Hyde, J., et al., Biochemistry, 2003, 42 6475-6483); and 1M48, 1M49, 1M4A, 1M4B, and 1M4C (Arkin, M. R., et al., Proc Natl Acad Sci USA, 2003, 100 1603-1608). Additionally, Stauber, D. J., et al, provides insight into the crystal structure of the IL-2 signaling complex: paradigm for a heterotrimeric cytokine receptor (Stauber, D. J., et al., PNAS, 2006, 103(8), 2788-2793).

Representative IL-2 Targeting Ligands are provided in FIG. 1. Additional IL-2 Targeting Ligands can be found in, for example, U.S. Pat. Nos. 8,802,721; 9,682,976, 9,708,268; Eur J Med Chem 83: 294-306 (2014), J Med Chem 60: 6249-6272 (2017); Nature 450: 1001-1009 (2007); each of which is incorporated by reference herein.

In certain embodiments the IL-2 Targeting Ligand is selected from

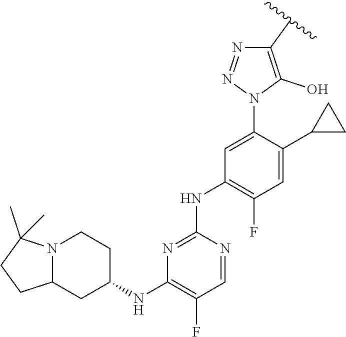 [SMILES: *c1nnn(-c2cc(Nc3ncc(F)c(N[C@H]4CCN5C(CCC5(C)C)C4)n3)c(F)cc2C2CC2)c1O]

### IL-6

In some embodiments, the Target Extracellular Protein is human interleukin-6 (IL-6) (UniProtKB—P05231 (IL6_HUMAN)). IL-6 is a cytokine with a wide variety of biological functions. It is a potent inducer of the acute phase response and plays an essential role in the final differentiation of B-cells into Ig-secreting cells. It is also involved in lymphocyte and monocyte differentiation. It also acts on B-cells, T-cells, hepatocytes, hematopoietic progenitor cells and cells of the CNS, and is required for the generation of T(H)17 cells. IL-6 has been implicated in a number of inflammatory diseases and cancers, including, but not limited to, Castleman's disease, metastatic castration-associated prostate cancer, renal cell carcinoma, large-cell lung carcinoma, ovarian cancer, rheumatoid arthritis, asthma.

The Protein Data Bank website provides the crystal structure of IL-6 searchable by 1P9M (Boulanger, M. J., et al., Science, 2003, 300: 2101-2104); 1ALU (Somers et al., EMBO J., 1997, 16, 989-997); 1IL6 and 2IL6 (Xu, G. Y., et al., J Mol Biol., 1997, 268 468-481) and 1N26 (Varghese et al., Proc Natl Acad Sci USA., 2002, 99 15959-15964); as well as the crystal structure of IL-6 bound to various compounds searchable by 4CNI (Shaw, S., et al., Mabs, 2014, 6: 773); and 4NI7 and 4NI9 (Gelinas et al., J Biol Chem. 2014, 289(12), 8720-8734). Additionally, Gelinas et al., provides insight into the crystal structure of interleukin-6 in complex with a modified nucleic acid ligand (Gelinas, A. D., et al., J Biol Chem. 2014, 289(12), 8720-8734); and Somers et al., provides insight into the crystal structure of interleukin 6: implications for a novel mode of receptor dimerization and signaling.

Potential IL-6 direct or indirect inhibitors are provided in FIG. 1. Additional potential IL-6 direct or indirect inhibitors can be found in, for example, U.S. Pat. No. 8,901,310; U.S. patent Ser. No. 10/189,796; U.S. Pat. No. 9,694,015; each incorporated herein by reference. In another embodiment the TL-6 Extracellular Targeting Ligand is AvimarC326 or a binding fragment thereof which is described in Nat Biotechnol 23, 1556-1561 (2005).

### IFN-γ

In some embodiments, the Target Extracellular Protein is human interferon-y (IFN-7) (UniProtKB—Q14609 (Q14609_HUMAN)). IFN-γ is a immunoregulatory cytokine. IFN-γ has been implicated in a number of autoimmune disorders, including, but not limited to rheumatoid arthritis, multiple sclerosis (MS), corneal transplant rejection, and various autoimmune skin diseases such as psoriasis, alopecia areata, vitiligo, acne vulgaris, and others.

The Protein Data Bank website provides the crystal structure of IFN-γ searchable by 1HIG (Ealick, S. E., et al., Science 252, 1991, 698-702); as well as the crystal structure of IFN-γ bound to various compounds searchable by 6E3K and 6E3L (Mendoza, J. L., et al., Nature, 2019, 567 56-60). Additionally, Randal et al., provides insight into the structure and activity of a monomeric interferon-γ: α-chain receptor signaling complex (Randal, M., et al., Structure, 2001, 9(2), 155-163).

Representative IFN-γ Targeting Ligands are described in FIG. 1. Additional IFN-7 Targeting Ligands can be found in, for example, J Med Chem 57: 4511-20 (2014); which is incorporated by reference herein.

### Tau Protein

In some embodiments, the Target Extracellular Protein is tau protein. The accumulation of tau in the brain causes aggregates that are associated with Alzheimer's and other tauopathies.

Non-limiting examples of Tau Protein targeting ligands include:

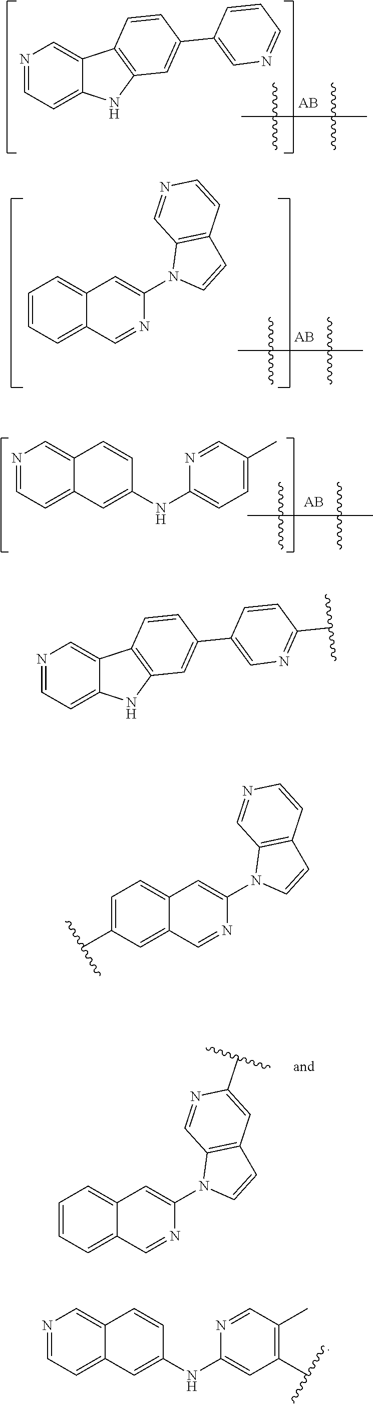

### IL-21

In some embodiments, the Target Extracellular Protein is human interleukin-21 (IL-21) (UniProtKB—Q9HBE4 (IL21_HUMAN)). IL-21 is an immunoregulatory cytokine. IL-21 has been implicated in a number of autoimmune disorders, including Sjögren's syndrome, systemic lupus erythematosus, type 1 diabetes, multiple sclerosis, rheumatoid arthritis, and inflammatory bowel disease.

The Protein Data Bank website provides the crystal structure of IL-21 searchable by 2OQP (Bondensgaard, K., et al., J Biol Chem., 2007, 282 23326-23336); and 4NZD (Hamming et al.); as well as the crystal structure of IL-21 bound to various compounds searchable by 3TGX (Hamming, 0. J., et al., J Biol Chem., 2012, 287(12), 9454-9460).

Representative IL-21 Targeting Ligands are described in FIG. 1. Additional IL-21 Targeting Ligands can be found in, for example, U.S. Pat. No. 9,701,663, which is incorporated herein by reference.

### IL-22

In some embodiments, the Target Extracellular Protein is human interleukin-22 (IL-22) (UniProtKB—Q9GZX6 (IL22 HUMAN)). IL-22 is a member of IL-10 family cytokines that is produced by many different types of lymphocytes including both those of the innate and adaptive immune system. IL-22 has been implicated in a number of autoimmune disorders, including, but not limited to, graft versus host disease (GVHD), psoriasis, rheumatoid arthritis, atopic dermatitis, and asthma.

The Protein Data Bank website provides the crystal structure of IL-22 searchable by 1M4R (Nagem, R. A. P., et al., Structure, 2002, 10 1051-1062); as well as the crystal structure of IL-22 bound to various compounds searchable by 3DGC (Jones, B. C. et al., Structure, 2008, 16 1333-1344).

Representative IL-22 Targeting Ligands are described in FIG. 1. Additional IL-22 Targeting Ligands can be found in, for example, U.S. Pat. No. 9,701,663, which is incorporated herein by reference.

### IL-10

In some embodiments, the Target Extracellular Protein is human interleukin-10 (IL-10) (UniProtKB—P22301 (IL10_HUMAN)). IL-10 is an inflammatory cytokine. IL-10 has been implicated in tumor survival and protection against cytotoxic chemotherapeutic drugs.

The Protein Data Bank website provides the crystal structure of IL-10 searchable by 2TLK (Zdanov, A et al., Protein Sci., 1996, 5 1955-1962); 1ILK (Zdanov, A. et al., Structure, 1995, 3 591-601); 2H24 (Yoon, S. I., et al., J Biol Chem., 2006, 281 35088-35096) and 3LQM (Yoon, S. I., et al., Structure, 2010, 18 638-648). Additionally, Zdanov, A., et al, provides insight into crystal structure of IL-10 (Zdanov A., Current Pharmaceutical design, 2004, 10, 3873-3884).

Representative IL-10 Targeting Ligands are provided in FIG. 1. Additional IL-10 Targeting Ligands can be found, for example, in ACS Chem Biol 11: 2105-11 (2016), which is incorporated herein by reference.

### IL-5

In some embodiments, the Target Extracellular Protein is human interleukin-5 (IL-5) (UniProtKB—P05113 (IL5_HUMAN)). IL-5 is a cytokine that regulates eosinophil maturation, recruitment, and survival. IL-5 has been implicated in a number of allergic disorders, including, but not limited to, asthma, nasal polyposis, atopic dermatitis, eosinophilic esophagitis, hypereosinophilic syndrome, and Churg-Strauss syndrome.

The Protein Data Bank website provides the crystal structure of IL-5 searchable by 1HUL (Milburn, M. V., Nature, 1993, 363, 172-176) and 3VA2 (Kusano et al., Protein Sci., 2012, 21(6) 850-864); as well as the crystal structure of IL-5 bound to various compounds searchable by 1OBX and 1OBZ (Kang, B. S., et al., Structure, 2003, 11, 845).

Representative IL-5 Targeting Ligands are provided in FIG. 1. Additional IL-5 Targeting Ligands can be found, for example, in Bioorg Med Chem 18: 4441-5 (2010); Bioorg Med Chem 18: 4625-9 (2011); Bioorg Med Chem 21: 2543-50 (2013); Eur J Med Chem 59: 31-8 (2013); Bioorg Med Chem 23: 2498-504 (2015); Bioorg Med Chem 20: 5757-62 (2012); each of which is incorporated by reference herein.

### IL8

In some embodiments, the Target Extracellular Protein is human interleukin-8 (IL-8) (UniProtKB—P10145 (IL8_HUMAN)). IL-8 is a chemotactic factor that attracts neutrophils, basophils, and T-cells, but not monocytes. It is also involved in neutrophil activation. It is released from several cell types in response to an inflammatory stimulus. IL-8 has been implicated in the promotion of tumor progression, immune escape, epithelial-mesenchymal transition, and recruitment of myeloid-derived suppressor cells. Studies have demonstrated that high serum IL-8 levels correlate with poor prognosis in many malignant tumors. Preclinical studies have shown that IL-8 blockade may reduce mesenchymal features in tumor cells, making them less resistant to treatment.

The Protein Data Bank website provides the crystal structure of IL-8 searchable by 3IL8 (Baldwin, E. T., et al., Proc Natl Acad Sci USA, 1991, 88, 502-506); and 1IL8 and 2IL8 (Clore, G. M., et al., Biochemistry, 1990, 29, 1689-1696); as well as the crystal structure of IL-8 bound to various compounds searchable by 1ILP and 1ILQ (Skelton, N, J., et al., Structure, 1999, 7, 157-168); and 1ROD (Sticht, H., et al., Eur J Biochem., 1996, 235, 26-35); 4XDX (Ostrov et al.,) and 5WDZ (Beckamp, S., J Biomol NMR, 2017, 69, 111-121).

Representative IL-8 Targeting Ligands are provided in FIG. 1. Additional IL-8 Targeting Ligands can be found in, for example, Bioorg Med Chem Lett 19: 4026-30 (2009), which is incorporated by reference herein.

### Cholinesterase

In some embodiments, the Target Extracellular Protein is human cholinesterase (UniProtKB—P06276 (CHLE_HUMAN)). Cholinesterase contributes to the inactivation of the neurotransmitter acetylcholine. Inhibition of cholinesterase results in increased levels of acetylcholine in the synaptic cleft (the space between two nerve endings). The main use of cholinesterase inhibitors is for the treatment of dementia in patients with Alzheimer's disease. People with Alzheimer's disease have reduced levels of acetylcholine in the brain. Cholinesterase inhibitors have been shown to have an effect on dementia symptoms such as cognition.

The Protein Data Bank website provides the crystal structure of cholinesterase searchable by 1P0I and 1P0Q (Nicolet, Y., et al., J Biol Chem., 2003, 278, 41141-41147); as well as the crystal structure of cholinesterase bound to various compounds searchable by 1P0M and 1P0P (Nicolet, Y., et al., J Biol Chem., 2003, 278, 41141-41147); 2J4C (Frasco, M. F., et al., FEBS J., 2007, 274 1849); 4BDT, 4BDS (Nachon, F., et al., Biochem J, 2013, 453, 393-399); 1GQR and 1GQS (Bar-on, P., et al., Biochemistry, 2002, 41, 3555); 3DJY and 3DKK (Carletti, E., et al., J Am Chem Soc., 2008, 130, 16011-16020); 4AXB, 4B00, 4BOP, and 4BBZ (Wandhammer, M., et al., Chem Biol Interact., 2013, 203, 19); 1DX6 (Greenblatt, H. M., et al., FEBS Lett., 1999, 463 321); 1GPK and 1GPN (Dvir, H., et al., Biochemistry, 2002, 41, 10810); 6CQY (Bester, S. M., et al., Chem Res Toxicol., 2018, 31, 1405-1417); 1XLV and 1XLW (Nachon, F., et al., Biochemistry, 2005, 44, 1154-1162); 2Y1K (Carletti, E., et al., Chem Res Toxicol., 2011, 24, 797); and 2WIG, 2WIJ, 2WIK, 2WIL, and 2WSL (Carletti, E., et al., Biochem J., 2009, 421, 97-106). Additionally, Ahmad et al., provides insight into the isolation, crystal structure determination and cholinesterase inhibitory potential of isotalatizidine hydrate from delphinium denudatum (Ahmad H., et al., Journal Pharmaceutical Biology, 2016, 55(1), 680-686).

Representative cholinesterase Targeting Ligands are provided in FIG. 1. Additional Targeting Ligands can be found in, for example, ACS Med Chem Lett 4: 1178-82 (2013); J Med Chem 49: 3421-5 (2006); Eur J Med Chem 55: 23-31 (2012); J Med Chem 51: 3154-70 (2008); J Med Chem 46: 1-4 (2002); Eur J Med Chem 126: 652-668 (2017); Biochemistry 52: 7486-99 (2013); Bioorg Med Chem 23: 1321-40 (2015); which are each incorporated herein by reference.

### C-C Motif Chemokine Ligand 2 (CCL2)

Grygiel et al., provides insight into the synthesis by native chemical ligation and crystal structure of human CCL2 (Grygiel, T. L., et al., Biopolymers, 2010, 94(3), 350-9).

In some embodiments, the Target Extracellular Protein is human C-C motif chemokine ligand 2 (CCL2) (UniProtKB—P13500 (CCL2_HUMAN)). CCL2 acts as a ligand for C-C chemokine receptor CCR2. CCL2 signals through binding and activation of CCR2 and induces a strong chemotactic response and mobilization of intracellular calcium ions. CCL2 exhibits a chemotactic activity for monocytes and basophils but not neutrophils or eosinophils.

CCL2 has been implicated in the recruitment of monocytes into the arterial wall during the disease process of atherosclerosis.

Representative CCL2 Targeting Ligands are provided in FIG. 1. Additional CCL2 Targeting Ligands can be found in, for example, J Med Chem 56: 7706-14 (2013), which is incorporated herein by reference.

### Carboxypeptidase B2

In some embodiments, the Target Extracellular Protein is human carboxypeptidase B2 (UniProtKB—Q96IY4 (CBPB2_HUMAN)). Carboxypeptidase B2, also known as thrombin activatable fibrinolysis inhibitor (TAFIa), cleaves C-terminal arginine or lysine residues from biologically active peptides such as kinins or anaphylatoxins in the circulation thereby regulating their activities. It down-regulates fibrinolysis by removing C-terminal lysine residues from fibrin that has already been partially degraded by plasmin. Carboxypeptidase B2 has been implicated and targeted to inhibit thrombosis.

The Protein Data Bank website provides the crystal structure of carboxypeptidase B2 (also known as thrombin-activatable fibrinolysis inhibitor (TAFI)) searchable by 3D66 (Marx, P. F., et al., Blood, 2008, 112, 2803-2809); 3DGV (Anand, K., et al., JBC, 2008, 283, 29416-29423); and 1KWM (Barbosa Pereira, P. J., et al., J Mol Biol., 2002, 321, 537-547); as well as the crystal structure of TAFI bound to various compounds searchable by 3D67 (Marx, P. F., et al., Blood, 2008, 112, 2803-2809); 5HVF, 5HVG, 5HVH (Zhou, X., et al., J Thromb Haemost., 2016, 14, 1629-1638); and 3LMS (Sanglas, L., et al., J Thromb Haemost., 2010, 8, 1056-1065). Additionally, Schreuder et al., provides insight into the interaction of TAFI and anabaenopeptin, a highly potent inhibitor of TAFI (Schreuder, H., et al., Sci Rep., 2016, 6, 32958).

Representative carboxypeptidase B2 Targeting Ligands are provided in FIG. 1. Additional carboxypeptidase B2 Targeting Ligands can be found in, for example, Bioorg Med Chem Lett 20: 92-6 (2010), J Med Chem 50: 6095-103 (2007), Bioorg Med Chem Lett 14: 2141-5 (2004), J Med Chem 58: 4839-44 (2015), J Med Chem 55: 7696-705 (2012), J Med Chem 59: 9567-9573 (2016), Bioorg Med Chem Lett 17: 1349-54 (2007), U.S. Pat. Nos. 9,662,310, 8,609,710, 9,688,645, J Med Chem 46: 5294-7 (2003), each of which is incorporated herein by reference.

### Neutrophil Elastase

In some embodiments, the Target Extracellular Protein is human neutrophil elastase (UniProtKB—P08246 (ELNE_HUMAN)). Neutrophil elastase modifies the functions of natural killer cells, monocytes and granulocytes. Inhibits C5a-dependent neutrophil enzyme release and chemotaxis.

Neutrophil elastase has been implicated in a number of disorders, including lung disease, chronic obstructive pulmonary disease, pneumonia, respiratory distress, and acute lung injury (ALI), and cystic fibrosis, as well as chronic kidney disease.

The Protein Data Bank website provides the crystal structure of human neutrophil elastase bound to various compounds searchable by 3Q76 and 3Q77 (Hansen, G., et al., J. Mol. Biol., 2011, 409, 681-691); 5ABW (Von Nussbaum, et al., Bioorg Med Chem Lett., 2015, 25, 4370-4381) 1BOF (Cregge, R. J., et al., J Med Chem., 1998, 41, 2461-2480); 1H1B (Macdonald, S. J. F., et al., J Med Chem., 2002, 45, 3878); 2Z7F (Koizumi, M., et al., J Synchrotron Radiat., 2008, 15 308-311); 5A09, 5AOA, 5AOB, and 5AOC (Von Nussbaum, F., et al., Chem Med Chem., 2015, 10, 1163-1173); 5A8X, 5A8Y and 5A8Z (Von Nussbaum, F., et al., Chem Med Chem., 2016, 11, 199-206); 1HNE (Navia, M. A., et al., Proc Natl Acad Sci USA, 1989, 86, 7-11); 6F5M (Hochscherf, J., et al., Acta Crystallogr F Struct Biol Commun., 2018, 74, 480-489); and 4WVP (Lechtenberg, B. C., et al., ACS Chem Biol., 2015, 10, 945-951).

Representative neutrophil elastase Targeting Ligands are provided in FIG. 1. Additional neutrophil elastase Targeting Ligands can be found in, for example, J Med Chem 53: 241-53 (2010), J Med Chem 38: 739-44 (1995), J Med Chem 37: 2623-6 (1994), J Med Chem 38: 4687-92 (1995), J Med Chem 45: 3878-90 (2002), Bioorg Med Chem Lett 5: 105-109 (1995), Bioorg Med Chem Lett 11: 243-6 (2001), J Med Chem 40: 1906-18 (1997), Bioorg Med Chem Lett 25: 4370-81 (2015), U.S. Pat. Nos. 8,569,314, 9,174,997, 9,290,457, each of which is incorporated herein by reference.

### Factor Xa

In some embodiments, the Target Extracellular Protein is human Factor Xa (UniProtKB—P00742 (FA10_HUMAN)). Factor Xa is a vitamin K-dependent glycoprotein that converts prothrombin to thrombin in the presence of factor Va, calcium and phospholipid during blood clotting.

Factor X has been implicated in the development of deep vein thrombosis and acute pulmonary embolism, and the risk of stroke and embolism in people with nonvalvular atrial fibrillation.

The Protein Data Bank website provides the crystal structure of Factor Xa bound to various compounds searchable by 1G2L and 1G2M (Nar, H., et al., Structure, 2001, 9, 29-38); 2PR3 (Nan huis, C. A., et al., Chem Biol Drug Des., 2007, 69, 444-450); 2UWP (Young, R. J., et al., Bioorg Med Chem Lett., 2007, 17, 2927); 2VVC, 2VVV, 2VVU, 2VWL, 2VWM, 2VWN and 2VWO (Zbinden, K. G., et al., Eur J Med Chem., 2009, 44, 2787); 4Y6D, 4Y71, 4Y7A, 4Y7B, 4zh8, 4ZHA (Convery, M. A. et al.); 4Y76, 4Y79, 2J94 and 2J95 (Chan, C., et al., J Med Chem., 2007, 50 1546-1557); 1FAX (Brandstetter, H., et al., J Biol Chem., 1996, 271, 29988-29992); 2JKH (Salonen, L. M., et al., Angew Chem Int Ed Engl., 2009, 48, 811); 2PHB (Kohrt, J. T., et al., Chem Biol Drug Des., 2007, 70, 100-112); 2W26 (Roehrig, S., et al., J Med Chem., 2005, 48, 5900) 2Y5F, 2Y5G and 2Y5H (Salonen, L. M., et al., Chemistry, 2012, 18, 213); 3Q3K (Yoshikawa, K., et al., Bioorg Med Chem Lett., 2011, 21, 2133-2140); 2BMG (Matter, K., et al., J Med Chem., 2005, 48, 3290); 2BOH, 2BQ6 2BQ7, and 2BQW (Nazare, M., et al., J Med Chem., 2005, 48, 4511); 2CJI (Watson, N. S., et al, Bioorg Med Chem Lett., 2006, 16, 3784); 2J2U, 2J34, 2J38, 2J41 (Senger, S., et al., Bioorg Med Chem Lett., 2006, 16 5731); 3IIT (Yoshikawa, K., et al., Bioorg Med Chem., 2009, 17 8221-8233); 1EZQ, 1FOR and 1FOS (Maignan, S., et al., J Med Chem., 2000, 43, 3226-3232); 1FJS (Adler, M., et al., Biochemistry, 2000, 39, 12534-12542); 1KSN (Guertin, K. R., et al., Bioorg Med Chem Lett., 2002, 12, 1671-1674); 1NFU, 1NFW, 1NFX and 1NFY (Maignan, S., et al., J Med Chem., 2003, 46, 685-690); 2XBV, 2XBW, 2XBX, 2XBY, 2XC0, 2XC4 and 2XC5 (Anselm, L., et al., Bioorg Med Chem Lett., 2010, 20, 5313); 4A7I (Nazare, M., et al., Angew Chem Int Ed Engl., 2012, 51, 905); 4BTI, 4BTT and 4BTU (Meneyrol, L., et al., J Med Chem., 2013, 56, 9441); 3FFG, 3KQB, 3KQC, 3KQD and 3KQE (Quan, M. L., et al., Bioorg Med Chem Lett., 2010, 20, 1373-1377); 2P93, 2P94 and 2P95 (Qiao, J. X., et al., Bioorg Med Chem Lett., 2007, 17, 4419-4427); 1V3X (Haginoya, N., et al., J Med Chem., 2004, 47, 5167-5182); 2P16 (Pinto, D. J. P., et al., J Med Chem., 2007, 50, 5339-5356); 2RAO (Lee, Y. K., et al., J Med Chem., 2008, 51, 282-297); 3SW2 (Shi, Y., et al., Bioorg Med Chem Lett., 2011, 21, 7516-7521); 2VH6 (Young, R. J., et al., Bioorg Med Chem Lett., 2008, 18, 23); 2WYG and 2WYJ (Kleanthous, S., et al., Bioorg Med Chem Lett., 2010, 20, 618); 2Y7X (Watson, N. S., et al., Bioorg Med Chem Lett., 2011, 21, 1588); 2Y7Z, 2Y80, 2Y81 and 2Y82 (Young, R. J., et al., Bioorg Med Chem Lett., 2011, 21, 1582); 3KL6 (Fujimoto, T., et al., J Med Chem., 2010, 53, 3517-3531) 3LIW (Meuller, M. M., et al., Biol. Chem., 2003, 383, 1185); 5KOH (Schweinitz, A., et al., Med Chem., 2006, 2, 349-361); 1XKA and 1XKB (Kamata, K., et al., Proc Natl Acad Sci USA, 1998, 95, 6630-6635); 2EI6 and 2EI7 (Nagata, T., et al., Bioorg Med Chem Lett., 2007, 17, 4683-4688) 2P3T (Ye, B., et al., J Med Chem., 2007, 50, 2967-2980); 1MQ5 and 1MQ6 (Adler, M., et al., Biochemistry, 2002, 41, 15514-15523); 3K9X and 3HPT (Shi, Y., et al., Bioorg Med Chem Lett., 2009, 19, 6882-6889); 3CEN (Corte, J. R., et al., Bioorg Med Chem Lett., 2008, 18, 2845-2849) 2W3I and 2W3K (Van Huis, C. A., et al., Bioorg Med Chem., 2009, 17, 2501); 2H9E (Murakami, M. T., et al., J Mol Biol., 2007, 366, 602-610); 1WU1 and 2D1J (Komoriya, S., et al., Bioorg Med Chem., 2005, 13, 3927-3954); 2G00 (Pinto, D. J. P., et al., Bioorg Med Chem Lett., 2006, 16, 5584-5589); 3M36 and 3M37 (Pruitt, J. R. et al., J Med Chem., 2003, 46, 5298-5315); 3CS7 (Qiao, J. X., et al., Bioorg Med Chem Lett., 2008, 18, 4118-4123); 1Z6E (Quan, M. L., et al., J Med Chem., 2005, 48, 1729-1744); 2FZZ (Pinto, D. J. P., et al., Bioorg Med Chem Lett., 2006, 16, 4141-4147); and 3ENS (Shi, Y., et al., J Med Chem., 2008, 51, 7541-7551).

Representative Factor Xa Targeting Ligands are provided in FIG. 1. Additional Factor Xa Targeting Ligands can be found in, for example, Bioorg Med Chem Lett 20: 5313-9 (2010), Bioorg Med Chem Lett 13: 679-83 (2003), J Med Chem 44: 566-78 (2001), J Med Chem 50: 2967-80 (2007), J Med Chem 38: 1511-22 (1995), Bioorg Med Chem Lett 18: 2845-9 (2008), J Med Chem 53: 6243-74 (2010), Bioorg Med Chem Lett 18: 2845-9 (2008), Bioorg Med Chem 16: 1562-95 (2008), each of which is incorporated herein by reference.

### Factor XI

In some embodiments, the Target Extracellular Protein is human Factor XI UniProtKB—P03951 (FA11_HUMAN). Factor XI triggers the middle phase of the intrinsic pathway of blood coagulation by activating factor IX.

Factor XI has been implicated in the development of deep vein thrombosis and acute pulmonary embolism, and the risk of stroke and embolism in people with nonvalvular atrial fibrillation.

The Protein Data Bank website provides the crystal structure of Factor XI bound to various compounds searchable by 1ZSL, 1ZTJ, 1ZTK, and 1ZTL (Nagafuji, P., et al.,); 1ZOM (Lin, J., et al., J Med Chem., 2006, 49, 7781-7791); 5EOK and 5EOD (Wong, S. S., et al., Blood, 2016, 127, 2915-2923); 1ZHM, 1ZHP and 1ZHR (Jin, L., et al., Acta Crystallogr D Biol Crystallogr., 2005, 61, 1418-1425); 1ZMJ, 1ZLR, 1ZML and 1ZMN (Lazarova, T. I., Bioorg Med Chem Lett., 2006, 16, 5022-5027); 1ZRK, 1ZSJ and 1ZSK (Guo, Z., et al); 4CRA, 4CRB, 4CRC, 4CRD, 4CRE, 4CRF and 4CRG (Fjellstrom, O., et al., PLoS One, 2015, 10, 13705); 3SOR and 3SOS (Fradera, X., et al., Acta Crystallogr Sect F Struct Biol Cryst Commun., 2012, 68, 404-408); 1ZPB, 1ZPC, 2FDA (Deng, H., et. al., Bioorg Med Chem Lett., 2006, 16, 3049-3054); 5WB6 (Wang, C., et al., Bioorg Med Chem Lett., 2017, 27, 4056-4060); 4NA7 and 4NA8 (Quan, M. L., et al., J Med Chem., 2014, 57, 955-969); 4WXI (Corte, J. R., et al., Bioorg Med Chem Lett., 2015, 25, 925-930); 5QTV, 5QTW, 5QTX and 5QTY (Fang, T., et al., Bioorg Med Chem Lett., 2020, 126949-126949); 6COS (Hu, Z., et al., Bioorg Med Chem Lett., 28, 987-992); 5QQP and 5QQO (Clark, C. G., et al., Bioorg Med Chem Lett., 2019, 29, 126604-126604); 5Q0D, 5Q0E, 5Q0F, 5Q0G, and 5Q0H (Corte, J. R., et al., Bioorg Med Chem Lett., 2017, 27, 3833-3839); 5QCK, 5QCL, 5QCM, and 5QCN (Pinto, D. J. P., et al., J Med Chem., 2017, 60, 9703-9723); 5TKS and 5TKU (Corte, J. R., et al., J Med Chem., 2017, 60, 1060-1075); 1XXD and 1XX9 (Jin, L., et al., J Biol Chem., 2005, 280, 4704-4712); 5QTT and 5QTU (Corte, J. R., et al., J Med Chem., 2019, 63, 784-803); 4TY6, 4TY7 (Hangeland, J. J., et al., J Med Chem., 2014, 57, 9915-9932); 4X6M, 4X6N, 4X60, and 4X6P (Pinto, D. J. P., et al., Bioorg Med Chem Lett., 2015, 25, 1635-1642); and 5EXM (Corte, J. R., et al., Bioorg Med Chem., 2016, 24, 2257-2272). Additionally, Al-Horani et al., provides insight into a review of patent literature regarding Factor Xia inhibitors (Al-Horani et al., Expert Opin Ther Pat. 2016; 26(3), 323-345).

Representative Factor XI Targeting Ligands are provided in FIG. 1. Additional Factor XI Targeting Ligands can be found in, for example, U.S. Pat. No. 9,783,530, U.S. patent Ser. No. 10/143,681, U.S. patent Ser. No. 10/214,512, ACS Med Chem Lett 6: 590-5 (2015), J Med Chem 60: 9703-9723 (2017), J Med Chem 60: 9703-9723 (2017), U.S. Pat. No. 9,453,018 (2016), J Med Chem 60: 1060-1075 (2017), J Med Chem 57: 955-69 (2014), each of which is incorporated herein by reference.

In certain embodiments the Factor XI Targeting Ligand is selected from:

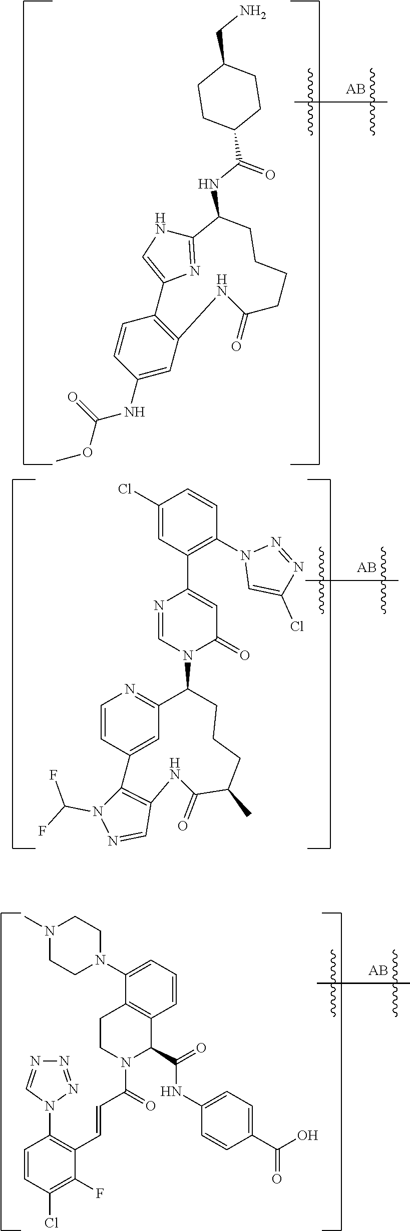

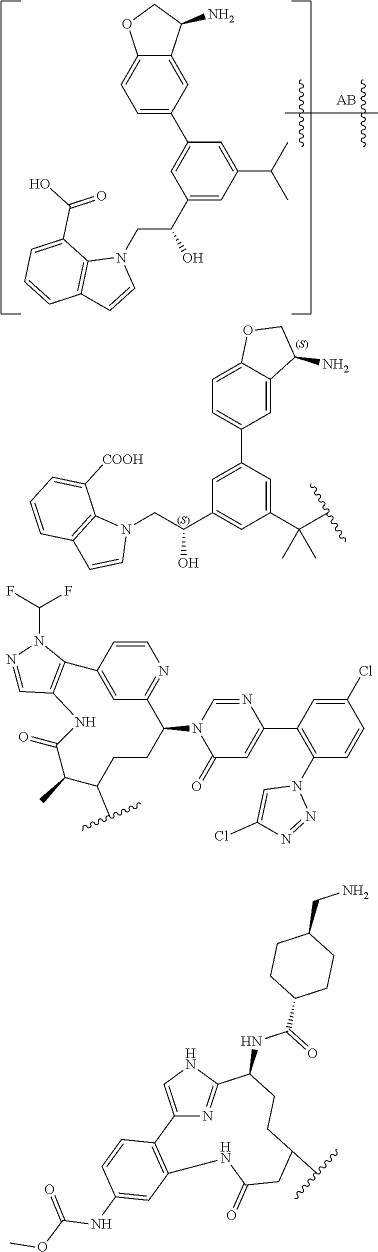

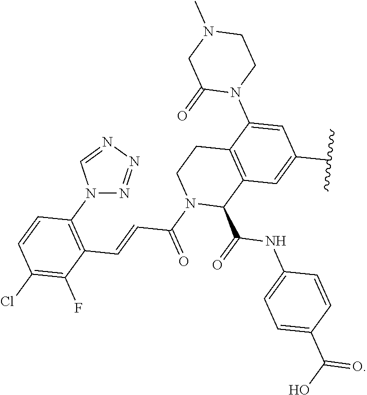 [SMILES: *c1cc2c(c(N3CCN(C)CC3=O)c1)CCN(C(=O)/C=C/c1c(-n3cnnn3)ccc(Cl)c1F)[C@@H]2C(=O)Nc1ccc(C(=O)O)cc1]

In certain embodiments the Factor XI Targeting Ligand is described in J Med Chem 61 (17), 7425-7447 (2018) or J Med Chem (2020) Structure-based design and pre-clinical characterization of selective and orally bioavailable Factor Xia inhibitors: demonstrating the power of an integrated Si protease family approach.

### Factor XII

In some embodiments, the Target Extracellular Protein is human Factor XII (UniProtKB—P00748 (FA12_HUMAN)). Factor XII is a serum glycoprotein that participates in the initiation of blood coagulation, fibrinolysis, and the generation of bradykinin and angiotensin. Prekallikrein is cleaved by factor XII to form kallikrein, which then cleaves factor XII first to alpha-factor XIIa and then trypsin cleaves it to beta-factor XIIa. Alpha-factor XIIa activates factor XI to factor XIa.

Factor XII has been implicated in the development of deep vein thrombosis and acute pulmonary embolism, and the risk of stroke and embolism in people with nonvalvular atrial fibrillation.

The Protein Data Bank website provides the crystal structure of factor XII bound to various compounds searchable by 4XDE and 4XE4 (Pathak, M., et al., J Thromb Haemost., 2015, 13(4) 580-591); 6GT6 and 6QF7 (Pathak, M., et al., Acta Crystallogr D Struct Biol., 2019, 75, 578-591); and 6B74 and 6B77 (Dementiev, A. A., et al., Blood Adv., 2018, 2, 549-558). Additionally, Pathak et al., provides insight into the crystal structure of factor XII (Pathak, M., et al., J Thromb Haemost., 2015, 13(4), 580-591).

Representative Factor XII Targeting Ligands are provided in FIG. 1. Additional Factor XII Targeting Ligands can be found in, for example, J Med Chem 60: 1151-1158 (2017), J Med Chem 48: 2906-15 (2005), J Med Chem 50: 5727-34 (2007), J Med Chem 50: 1876-85 (2007), Chembiochem 18: 387-395 (2017), each of which is incorporated herein by reference.

### Factor XIII

In some embodiments, the Target Extracellular Protein is human Factor XIII UniProtKB—P00488 (F13A_HUMAN)). Factor XIII is activated by thrombin and calcium ion to a transglutaminase that catalyzes the formation of gamma-glutamyl-epsilon-lysine cross-links between fibrin chains, thus stabilizing the fibrin clot. Also cross-link alpha-2-plasmin inhibitor, or fibronectin, to the alpha chains of fibrin.

Factor XIII has been implicated in the development of deep vein thrombosis and acute pulmonary embolism, and the risk of stroke and embolism in people with nonvalvular atrial fibrillation.

The Protein Data Bank website provides the crystal structure of factor XIII searchable by 1FIE (Yee, V. C., et al., Thromb Res., 1995, 78, 389-397); and 1F13 (Weiss, M. S., et al., FEBS Lett., 1998, 423, 291-296); as well as the crystal structure of factor XIII bound to various compounds searchable by 1DE7 (Sadasivan, C., et al., J Biol Chem., 2000, 275, 36942-36948); and 5MHL, 5MHM, 5MHN, and 5MHO (Stieler, M., et al.,). Additionally, Gupta et al., provides insight into the mechanism of coagulation factor XIII activation and regulation from a structure/functional perspective (Gupta, S., et al., Sci Rep., 2016; 6, 30105); and Komaromi et al., provides insight into the novel structural and functional aspect of factor XIII (Komaromi, Z., et al., J Thromb Haemost 2011, 9, 9-20).

Representative Factor XIII Targeting Ligands are provided in FIG. 1. Additional Factor XIII Targeting Ligands can be found in, for example, Eur J Med Chem 98: 49-53 (2015), J Med Chem 55: 1021-46 (2012), J Med Chem 48: 2266-9 (2005), each of which is incorporated herein by reference.

### Prothrombin

In some embodiments, the Target Extracellular Protein is human Prothrombin (UniProtKB—P00734 (THRB_HUMAN)). Thrombin, which cleaves bonds after Arg and Lys, converts fibrinogen to fibrin and activates factors V, VII, VIII, XIII, and, in complex with thrombomodulin, protein C. Functions in blood homeostasis, inflammation and wound healing.

Thrombin is involved in blood clot formation and arterial and venous thrombosis, and thromboembolism associated with atrial fibrillation.

The Protein Data Bank website provides the crystal structure of prothrombin searchable by 3NXP (Chen, Z. et al., Proc Natl Acad Sci USA, 2010, 107, 19278-19283); as well as the crystal structure of prothrombin bound to various compounds searchable by 2HPP and 2HPQ (Arni, R. K., et al., Biochemistry, 1993, 32, 4727-4737); 6BJR, 6C₂W (Chinnaraj, M., et al., Sci Rep., 2018, 8, 2945-2945); 5EDK, 5EDM (Pozzi, N., et al., J Biol Chem., 2016, 291, 6071-6082); 3K65 (Adams, T. E., et al., Biochimie, 2016, 122, 235-242); and 6BJR and 6C₂W (Chinnaraj, M. et al., Sci Rep., 2018, 8, 2945-2945). Additionally, Pozzi et al., provides insight into the mechanism and conformational flexibility for the crystal structure of prothrombin (Pozzi, N. et al., J Biol Chem., 2013, 288(31), 22734-22744); and Zhiwei et al., provides insight into the crystal structure of prothrombin-1 (Zhiwei, C. et al., PNAS, 2010, 107(45), 19278-19283).

Prothrombin is converted to thrombin, as such the Protein Data Bank website provides the crystal structure of thrombin bound to compounds searchable by 1XMN (Carter, W. J. et al., J. Biol. Chem., 2005, 280, 2745-2749); 4CH₂ and 4CH₈ (Lechtenberg, B. C. et al., J Mol Biol., 2014, 426, 881); 3PO1 (Karle, M. et al., Bioorg Med Chem Lett., 2012, 22, 4839-4843); 3DA9 (Nilsson, M. et al., J Med Chem., 2009, 52, 2708-2715); 2H9T and 3BF6 (Lima, L. M. T. R. et al., Biochim Biophys Acta., 2009, 1794, 873-881); 3BEF and 3BEI (Gandhi, P. S. et al., Proc Natl Acad Sci USA, 2008, 105, 1832-1837); 3BV9 (Nieman, M. T. et al., J Thromb Haemost., 2008, 6, 837-845); 2HWL (Pineda, A. O. et al., Biophys Chem., 2007, 125, 556-559); 2AFQ (Johnson, D. J. D. et al., Biochem J., 2005, 392, 21-28); 1SHH (Pineda, A. O. et al., J Biol Chem., 2004, 279, 31842-31853); 1JWT (Levesque, S. et al., Bioorg Med Chem Lett., 2001, 11, 3161-3164); 1G37 (Bachand, B. et al., Bioorg Med Chem Lett., 2001, 11, 287-290); 1EOJ and 1EOL (Slon-Usakiewicz, J. J. et al., Biochemistry, 2000, 39, 2384-2391); 1AWH (Weir, M. P. et al., Biochemistry, 1998, 37, 6645-6657); 1DIT (Krishnan, R. et al., Protein Sci., 1996, 5, 422-433) 1HAO and 1HAP (Padmanabhan, K. et al., Acta Crystallogr D Biol Crystallogr., 1996, 52, 272-282); and 1HBT (Rehse, P. H. et al., Biochemistry, 1995, 34, 11537-11544).

Representative prothrombin Targeting Ligands are provided in FIG. 1. Additional prothrombin Targeting Ligands can be found in, for example, J Med Chem 46: 3612-22 (2003), Bioorg Med Chem Lett 12: 1017-22 (2002), J Med Chem 40: 830-2 (1997), Bioorg Med Chem Lett 15: 2771-5 (2005), J Med Chem 42: 3109-15 (1999), J Med Chem 47: 2995-3008 (2004), Bioorg Med Chem 16: 1562-95 (2008), J Med Chem 42: 3109-15 (1999), each of which is incorporated herein by reference.

### Coagulation Factor VII

In some embodiments, the Target Extracellular Protein is human coagulation Factor VII (UniProtKB—P08709 (FA7_HUMAN)). Factor VII initiates the extrinsic pathway of blood coagulation. It is a serine protease that circulates in the blood in a zymogen form. Factor VII is converted to Factor VIIa by Factor Xa, Factor XIIa, Factor IXa, or thrombin by minor proteolysis. In the presence of tissue factor and calcium ions, Factor VIIa then converts Factor X to Factor Xa by limited proteolysis. Factor VIIa will also convert Factor IX to Factor IXa in the presence of tissue factor and calcium.

Factor VII is involved in blood clot formation and arterial and venous thrombosis, and thromboembolism associated with atrial fibrillation.

The Protein Data Bank website provides the crystal structure of factor VII bound to various compounds searchable by 2F9B (Rai, R., et al., Bioorg Med Chem Lett., 2006, 16, 2270-2273) 5U6J (Wurtz, N. R., et al., Bioorg Med Chem Lett., 2017, 27, 2650-2654); 5L2Y, 5L2Z, and 5L30 (Ladziata, .U., et al., Bioorg Med Chem Lett., 2016, 26, 5051-5057); 5146 (Glunz, P. W., et al., J Med Chem., 2016, 59, 4007-4018); 4YLQ, 4Z6A, and 4ZMA (Sorensen, A. B., et al., J Biol Chem., 2016, 291, 4671-4683); 4YT6 and 4YT7 (Glunz, P. W., et al., Bioorg Med Chem Lett, 2015, 25, 2169-2173); 4NA9 (Quan, M. L., et al., J Med Chem., 2014, 57, 955-969); 4NG9 (hang, X., et al., ACS Med Chem Lett., 2014, 5, 188-192); 4JZD, 4JZE and 4JZF (Bolton, S. A., et al., Bioorg Med Chem Lett., 2013, 23, 5239-5243); 4JYU and 4JYV (Glunz, P. W., et al., Bioorg Med Chem Lett., 2013, 23, 5244-5248); 4ISH (Priestley, E. S., et al., Bioorg Med Chem Lett., 2013, 23, 2432-2435) 4ISI (Zhang, X., et al., Bioorg Med Chem Lett., 2013, 23, 1604-1607); 2ZZU (Shiraishi, T., et al., Chem Pharm Bull (Tokyo), 2010, 58, 38-44); 1WV7 and 1WUN (Kadono, S., et al., Biochem Biophys Res Commun., 2005, 327, 589-596); 2ZWL, 2ZP0, (Kadono, S., et al.); 2EC9 (Krishan, R., et al., Acta Crystallogr D Biol Crystallogr., 2007, 63, 689-697); 2PUQ (Larsen, K. S., et al., Biochem J., 2007, 405, 429-438); 2FLR (Riggs, J. R., et al., Bioorg Med Chem Lett., 2006, 16, 3197-3200); 2C₄F (Kohrt, J. T., et al., Bioorg Med Chem Lett., 2006, 16, 1060); 2AEI (Kohrt, J. T. et al., Bioorg Med Chem Lett., 2005, 15, 4752-4756); 1WTG (Kadono, S., et al., Biochem Biophys Res Commun., 2005, 326, 859-865); 1WSS (Kadono, S., et al., Acta Crystallogr Sect F Struct Biol Cryst Commun., 2005, 61, 169-173); 1W7X and 1W8B (Zbinden, K. G., et al., Bioorg Med Chem Lett., 2005, 15, 5344); 1WQV (Kadono, S., et al., Biochem Biophys Res Commun., 2004, 324, 1227-1233); 1Z6J (Schweitzer, B. A., et al., Bioorg Med Chem Lett., 2005, 15, 3006-3011); 1YGC (Olivero, A. G., et al., J Biol Chem., 2005, 280, 9160-9169); 6R2W (Sorensen, A. B., et al., J Biol Chem., 2019, 295, 517-528); 5PA8, 5PA9, 5PAA, 5PAB, 5PAC, 5PAE, 5PAF, 5PAG, 5PAI, 5PAJ, 5PAK, 5PAM, 5PAN, 5PAO, 5PAQ, 5PAR, 5PAS, 5PAT, 5PAU, 5PABV, 5PAW, 5PAX, 5PAY, 5PBO, 5PB1, 5PB2, 5PB3, 5PB4, 5PB5, and 5PB6 (Mayweg, A. V., et al.,); and 5LOS (Li, Z., et al., Nat Commun., 2017, 8, 185-185). Additionally, Kemball-Cook, et al., provides insight into the crystal structure of active site-inhibited factor VIIa (Kemball-Cook, G., et al., J Struct Biol., 1999, 127(3), 213-23).

Representative Factor VII Targeting Ligands are provided in FIG. 1. Additional Factor VII Targeting Ligands can be found in, for example, U.S. Pat. No. 9,174,974, Bioorg Med Chem Lett 26: 5051-5057 (2016), Bioorg Med Chem Lett 11: 2253-6 (2001), Bioorg Med Chem Lett 15: 3006-11 (2005), Bioorg Med Chem Lett 12: 2883-6 (2002), each of which is incorporated herein by reference.

### Coagulation Factor IX

In some embodiments, the Target Extracellular Protein is human coagulation Factor IX (UniProtKB—P00740 (FA9_HUMAN)). Factor IX Factor IX is a vitamin K-dependent plasma protein that participates in the intrinsic pathway of blood coagulation by converting factor X to its active form in the presence of Ca2+ ions, phospholipids, and factor VIIIa.

Factor IX is involved in blood clot formation and arterial and venous thrombosis, and thromboembolism associated with atrial fibrillation.

The Protein Data Bank website provides the crystal structure of factor IX bound to various compounds searchable by 6MV4 (Vadivel, K., et al., J Thromb Haemost., 2019, 17, 574-584) 4ZAE (Zhang, T., et al., Bioorg Med Chem Lett., 2015, 25, 4945-4949); 4YZU and 4Z0K (Parker, D. L., et al., Bioorg Med Chem Lett., 2015, 25, 2321-2325); 5TNO and 5TNT (Sakurada, I., et al., Bioorg Med Chem Lett., 2017, 27, 2622-2628); 5JB8, 5JB9, 5JBA, 5JBB and 5JBC (Kristensen, L. H., et al., Biochem J., 2016, 473, 2395-2411); 3LC3 (Wang, S., et al., J Med Chem., 2010, 53, 1465-1472); 3LC5 (Wang, S., et al., J Med Chem., 2010, 53, 1473-1482); 3KCG (Johnson, D. J. D., et al., Proc Natl Acad Sci USA, 2010, 107, 645-650); 1NL0 (Huang, M., et al., J Biol Chem., 2004, 279, 14338-14346); 1RFN (Hopfner, K. P., et al., Structure, 1999, 7, 989-996); and 6RFK (Sendall, T. J., et al.,).

Representative Factor IX Targeting Ligands are provided in FIG. 1. Additional Factor IX Targeting Ligands can be found in, for example, U.S. Pat. No. 9,409,908, Bioorg Med Chem Lett 25: 5437-43 (2015), U.S. patent Ser. No. 10/189,819, each of which is incorporated herein by reference.

### Fibroblast Growth Factor 1 (FGF1)

In some embodiments, the Target Extracellular Protein is human fibroblast growth factor 1 (FGF1) (UniProtKB—P05230 (FGF1_HUMAN)). FGF1 plays an important role in the regulation of cell survival, cell division, angiogenesis, cell differentiation and cell migration. FGF1 acts as a ligand for FGFR1 and integrins, and binds to FGFR1 in the presence of heparin leading to FGFR1 dimerization and activation via sequential autophosphorylation on tyrosine residues which act as docking sites for interacting proteins, leading to the activation of several signaling cascades. FGF1 induces the phosphorylation and activation of FGFR1, FRS2, MAPK3/ERK1, MAPK1/ERK2 and AKT1. FGF1 can induce angiogenesis. FGF1 has been implicated in oncogenesis, cancer cell proliferation, resistance to anticancer therapies, and neoangiogenesis.

The Protein Data Bank website provides the crystal structure of FGF1 searchable by 2AFG (Blaber, M., et al., Biochemistry, 1996, 35, 2086-2094); and 1BAR (Zhu, X. et al., Science, 1991, 251, 90-93); as well as the crystal structure of FGF1 bound to various compounds searchable by 1AFC (Zhu, X., et al., Structure, 1993, 1, 27-34); 1AXM and 2AXM (DiGabriele, A. D., et al., Nature, 1998, 393, 812-817); IEVT (Plotnikov, A. N., et al., Cell, 2000, 101, 413-424); 1E00 (Pellegrini, L., et al., Nature, 2000, 407, 1029); and 2ERM (Canales, A., et al., FEBS J, 2006, 273, 4716-4727).

Representative FGF1 Targeting Ligands are provided in FIG. 1. Additional FGF1 Targeting Ligands can be found in, for example, Bioorg Med Chem Lett 18: 344-9 (2008), Chembiochem 6: 1882-90 (2005), J Med Chem 55: 3804-13 (2012), J Med Chem 47: 1683-93 (2004), J Med Chem 53: 1686-99 (2010,) each of which is incorporated herein by reference.

### Fibroblast Growth Factor 2 (FGF2)

In some embodiments, the Target Extracellular Protein is human fibroblast growth factor 2 (FGF2) (UniProtKB—P09038 (FGF2_HUMAN)). FGF2 acts as a ligand for FGFR1, FGFR2, FGFR3 and FGFR4. FGF2 also acts as an integrin ligand which is required for FGF2 signaling, and plays an important role in the regulation of cell survival, cell division, cell differentiation and cell migration. FGF2 also induces angiogenesis. FGF2 has been implicated in oncogenesis, cancer cell proliferation, resistance to anticancer therapies, and neoangiogenesis.

The Protein Data Bank website provides the crystal structure of FGF2 bound to various compounds searchable by 40EE, 4OEF, and 4OEG (Li, Y. C., et al., ACS Chem Biol., 2014, 9, 1712-1717); 1EV2 (Plotnikov, A. N., et al., Cell, 2000, 101, 413-424); and 5X10 (Tsao, Y. H.).

Representative FGF2 Targeting Ligands are provided in FIG. 1. Additional FGF2 Targeting Ligands can be found in, for example, U.S. Pat. No. 8,933,099, Bioorg Med Chem Lett 12: 3287-90 (2002), Chem Biol Drug Des 86: 1323-9 (2015), Bioorg Med Chem Lett 25: 1552-5 (2015), each of which is incorporated herein by reference.

### Fibronectin-1

In some embodiments, the Target Extracellular Protein is human fibronectin 1 (FN1) (UniProtKB—P02751 (FINC_HUMAN)). Fibronectin (FN) polymerization is necessary for collagen matrix deposition and is a key contributor to increased abundance of cardiac myofibroblasts (MFs) after cardiac injury. Interfering with FN polymerization may attenuate MF and fibrosis and improve cardiac function after ischemia/reperfusion (I/R) injury.

The Protein Data Bank website provides the crystal structure of fibronectin-1 bound to various compounds searchable by 3M7P (Graille, M., et al., Structure, 2010, 18, 710-718); 3MQL (Erat, M. C., et al., J Biol Chem., 2010, 285, 33764-33770); and 3EJH (Erat, M. C., et al., Proc Natl Acad Sci USA, 2009, 106, 4195-4200).

Representative FN Targeting Ligands are provided in FIG. 1. Additional FN Targeting Ligands can be found in, for example, Bioorg Med Chem Lett 18: 2499-504 (2008), which is incorporated herein by reference.

### Kallikrein-1 (KLK1)

In some embodiments, the Target Extracellular Protein is human kallikrein-1 (UniProtKB—P06870 (KLK1_HUMAN)). Glandular kallikreins cleave Met-Lys and Arg-Ser bonds in kininogen to release Lys-bradykinin. Kallikrein has been implicated in adverse reactions in hereditary angioedema (HAE).

The Protein Data Bank website provides the crystal structure of KLK1 searchable by 1SPJ (Laxmikanthan, G., et al., Proteins, 2005, 58, 802-814); as well as the crystal structure of KLK1 bound to various compounds searchable by 5F8Z, 5F8T, 5F8X, (Xu, M., et al.,); and 6A80 (Xu, M., et al., FEBS Lett., 2018, 592, 2658-2667). Additionally, Katz et al., provides insight into the crystal structure of kallikrein (Katz, B. A., et al., Protein Sci., 1998, 7(4), 875-85).

Representative kallikrein Targeting Ligands are provided in FIG. 1. Additional kallikrein Targeting Ligands can be found in, for example, U.S. Pat. No. 9,783,530, J Med Chem 38: 2521-3 (1995), U.S. Pat. No. 9,234,000, U.S. patent Ser. No. 10/221,161, U.S. Pat. Nos. 9,687,479, 9,670,157, 9,834,513, J Med Chem 38: 1511-22 (1995), U.S. patent Ser. No. 10/214,512, each of which is incorporated herein by reference.

### Plasma Kallikrein

In some embodiments, the Target Extracellular Protein is human plasma kallikrein (UniProtKB—P03952 (KLKB1_HUMAN)). Plasma kallikrein cleaves Lys-Arg and Arg-Ser bonds. It activates, in a reciprocal reaction, factor XII after its binding to a negatively charged surface. It also releases bradykinin from HMW kininogen and may also play a role in the renin-angiotensin system by converting prorenin into renin. Plasma kallikrein has been implicated in retinal dysfunction, the development of diabetic macular edema and hereditary angioedema (HAE).

The Protein Data Bank website provides the crystal structure of plasma kallikrein bound to various compounds searchable by 5TJX (Li, Z., et al., ACS Med Chem Lett., 2017, 8, 185-190) 6O1G and 6O1S (Patridge, J. R., et al., J Struct Biol., 2019, 206, 170-182); 4OGX and 4OGY (Kenniston, J. A., et al., J Biol Chem., 2014, 289, 23596-23608); and 5F8T, 5F8X, and 5F8Z (Xu, M., et al.,).

Representative plasma kallikrein Targeting Ligands are provided in FIG. 1. Additional plasma kallikrein Targeting Ligands can be found in, for example, J Med Chem 61: 2823-2836 (2018), J Med Chem 55: 1171-80 (2012), U.S. Pat. Nos. 8,598,206, 9,738,655, Bioorg Med Chem Lett 16: 2034-6 (2006), U.S. Pat. No. 9,409,908, U.S. patent Ser. No. 10/144,746, U.S. Pat. No. 9,290,485, each of which is incorporated herein by reference.

### Lipoprotein Lipase

In some embodiments, the Target Extracellular Protein is human lipoprotein lipase (UniProtKB—P06858 (LIPL_HUMAN)). Lipoprotein lipase is a key enzyme in triglyceride metabolism. It catalyzes the hydrolysis of triglycerides from circulating chylomicrons and very low density lipoproteins (VLDL), and thereby plays an important role in lipid clearance from the blood stream, lipid utilization and storage. Lipoprotein lipase mediates margination of triglyceride-rich lipoprotein particles in capillaries. Lipoprotein lipase has been implicated in the development of cardiovascular disease and obesity.

The Protein Data Bank website provides the crystal structure of lipoprotein lipase bound to various compounds searchable by 6E7K (Birrane, G., et al., Proc Natl Acad Sci USA, 2018 116 1723-1732).

Representative lipoprotein lipase Targeting Ligands are provided in FIG. 1. Additional lipoprotein lipase Targeting Ligands can be found in, for example, J Med Chem 47: 400-10 (2004), which is incorporated herein by reference.

### Matrix Metallopeptidase 1 (MMP-1)

In some embodiments, the Target Extracellular Protein is human matrix metallopeptidase 1 (MMP-1) (UniProtKB—P03956 (MMP1_HUMAN)). MMP-1 cleaves collagens of types I, II, and III at one site in the helical domain. It also cleaves collagens of types VII and X. MMP-1 has been implicated in cardiovascular disease.

The Protein Data Bank website provides the crystal structure of MMP-1 searchable by 3SHI (Bertini, I., et al., FEBS Lett., 2012, 586, 557-567); as well as the crystal structure of MMP-1 bound to various compounds searchable by 4AUO (Manka, S. W., et al., Proc Natl Acad Sci USA, 2012, 109, 12461); 3MA2 (Grossman, M., et al., Biochemistry, 2010, 49, 6184-6192); and 2JOT (Iyer, S., et al., J. Biol. Chem., 2007, 282, 364). Additionally, Iyer et al., provides insight into the crystal structure of an active form of MMP-1 (Iyer, S., et al., J Mol Biol., 2006, 362(1), 78-88); and Lovejoy et al., provides insight into the crystal structure of MMP1 and the selectivity of collagenase inhibitors (Lovejoy, B., et al., Nat Struct Mol Biol., 1999, 6, 217-221).

Representative MMP-1 Targeting Ligands are provided in FIG. 1. Additional MMP-1 Targeting Ligands can be found in, for example, Bioorg Med Chem Lett 5: 1415-1420 (1995), Bioorg Med Chem Lett 16: 2632-6 (2006), Bioorg Med Chem Lett 8: 837-42 (1999), Eur J Med Chem 60: 89-100 (2013), J Med Chem 54: 4350-64 (2011), Bioorg Med Chem Lett 8: 3251-6 (1999), J Med Chem 42: 4547-62 (1999), J Med Chem 61: 2166-2210 (2018), J Med Chem 41: 1209-17 (1998), which is incorporated herein by reference.

### Macrophage Migration Inhibitory Factor (MIF)

In some embodiments, the Target Extracellular Protein is human macrophage migration inhibitory factor (MIF) (UniProtKB—P14174 (MIF_HUMAN)). MIF is a pro-inflammatory cytokine involved in the innate immune response to bacterial pathogens. The expression of MIF at sites of inflammation suggests a role as mediator in regulating the function of macrophages in host defense. It counteracts the anti-inflammatory activity of glucocorticoids.

MIF has been implicated in tumor progression; systemic inflammation; atherosclerosis; rheumatoid arthritis; and systemic lupus erythematosus, among others.

The Protein Data Bank website provides the crystal structure of MIF searchable by 1MIF (Sun, H-W. et al., Proc Natl Acad Sci USA, 1996, 93, 5191-5196); as well as the crystal structure of MIF bound to various compounds searchable by 6PEG (Cirillo, P. F. et al.,); 5XEJ (Fukushima, K); 6FVE and 6FVH (Sokolov, A. V., et al., Biochemistry (Mosc), 2018, 83, 701-707); 6CB5, 6CBF, 6CBG, and 6CBH (Trivedi-Parmar, V., et al., Chem Med Chem., 2018, 13, 1092-1097) 6B1C, 6B1K, 6B2C, (Dawson, T. K., et al., ACS Med Chem Lett., 2017, 8, 1287-1291); 4Z15, 4Z1T and 4Z1U (Singh, A. K., et al, J Cell Mol Med., 2017, 21, 142-153); 5HVS and 5HVT (Cisneros, J. A., et al., J Am Chem Soc., 2016, 138, 8630-8638); 4PKK (Pantouris, G., et al.,); 5J7P and 5J7Q (Cisneros, J. A., et al., Bioorg Med Chem Lett., 2016, 26, 2764-2767); 5B40 (Kimura, H., et al., Chem Biol., 2010, 17, 1282-1294); 4PLU, 4TRF, 4POH, and 4P01 (Pantouris, G., et al., Chem Biol., 2015, 22, 1197-1205); 4WR8 and 4WRB (Dziedzic, P., et al., J Am Chem Soc., 2015, 137 2996-3003); 4K9G (Ioannou, K., et al., Int J Oncol., 2014, 45, 1457-1468); 40SF, 3WNR, 3WNS and 3WNT (Spencer, E. S., et al., Eur J Med Chem., 2015, 93, 501-510); 40YQ (Spencer, E. S. et al.,); 3SMB and 3SMC (Crichlow, G. V. et al., Biochemistry, 2012, 51, 7506-7514); 3U18 (Bai, F., et al., J Biol Chem., 2012, 287, 30653-30663); 4F2K (Tyndall, J. D. A., et al., Acta Crystallogr Sect F Struct Biol Cryst Commun., 2012, 68, 999-1002); 3IJG and 3IJJ (Cho, Y., et al., Proc Natl Acad Sci USA, 2010, 107, 11313-11318); 3L5P, 3L5R, 3L5S, 3L5T, 3L5U, and 3L5V (McLean, L. R. et al., Bioorg Med Chem Lett., 2010, 20, 1821-1824); 3JSF, 3JSG and 3JTU (McLean, L. R., et al., Bioorg Med Chem Lett., 2009, 19, 6717); 3HOF (Crawley, L., et al.); 3CE4 and 3DJI (Crichlow G. V., et al., Biochemistry, 2009, 48, 132-139); 3B9S (Winner, M. et al., Cancer Res., 2008, 68, 7253-7257); 200H, 20OW and 200Z (Crichlow, G. V. et al., J Biol Chem., 2007, 282, 23089-23095); 1GCZ and 1GD0 (Orita, M. et al., J Med Chem., 2001, 44, 540-547); and 1CA7, 1CGQ and 1P1G (Lubetsky, J. B. et al., Biochemistry, 1999, 38, 7346-7354). Additionally, Sun et al., provides insight into the crystal structure of MIF (Proc Natl Acad Sci USA., 1996, 28; 93(11), 5191-6).

Representative MIF Targeting Ligands are provided in FIG. 1. Additional MIF Targeting Ligands can be found in, for example, ACS Med Chem Lett 8: 124-127 (2017), J Med Chem 44: 540-7 (2001), J Med Chem 52: 416-24 (2009), J Med Chem 50: 1993-7 (2007), which is incorporated herein by reference.

### Transforming Growth Factor-β2 (TGF-β2)

In some embodiments, the Target Extracellular Protein is human transforming growth factor-β2 (TGF-β2) (UniProtKB—P61812 (TGFB2_HUMAN)). TGF-β2 is a multifunctional protein that regulates various processes such as angiogenesis and heart development. Once activated following release of LAP, TGF-beta-2 acts by binding to TGF-beta receptors (TGFBR1 and TGFBR2), which transduce signal. TGF-β2 expression in the tumor microenvironment has been associated with a poor prognosis, and is implicated in TGF-β2 mediated tumor suppression via T-cell exclusion. TGF-β2 expression has also been implicated in hematological malignancies and fibrosis.

The Protein Data Bank website provides the crystal structure of TGF-β2 searchable by 6I9J (Del Amo-Maestro L. et al., Sci Rep. 2019, 9, 8660-8660); as well as the crystal structure of TGF-32 bound to various compounds searchable by 1M9Z (Boesen, C. C., et al. Structure, 2002, 10, 913-919); 5QIN (Zhang, Y. et al., ACS Med Chem Lett., 2018, 9, 1117-1122); 5E8V, 5E8Y, 5E91 and 5E92 (Tebben, A. J. et al., Acta Crystallogr D Struct Biol., 2016, 72, 658-674); 4P7U (Wangkanont, K. et al., Protein Expr Purif, 2015, 115, 19-25); 4XJJ (Wangkanont et al.); and 1KTZ (Hart, P. J., et al., Nat Struct Biol., 2002, 9, 203-208).

Representative TGF-β2 Targeting Ligands are provided in FIG. 1.

### Thrombospondin-1 (TSP-1)

In some embodiments, the Target Extracellular Protein is human thrombospondin-1 (TSP-1) (UniProtKB—P61812 (TGFB2_HUMAN)). TSP1 acts as an angiogenesis inhibitor by stimulating endothelial cell apoptosis, inhibiting endothelial cell migration and proliferation, and regulating vascular endothelial growth factor bioavailability and activity. TSP1 affects tumor immune response, tumor cell behaviors including adhesion, invasion, migration, apoptosis, and proliferation.

TSP-1 expression has been implicated in a number of diseases, including in promoting certain cancers such as breast cancer, prostate cancer, melanoma, SCLC, osteosarcoma, cutaneous squamous cell carcinoma, oral squamous cell carcinoma, papillary thyroid carcinoma, thyroid cancer, medulloblastoma, and fibrotic disorders such as diabetes, liver fibrosis, and in multiple myeloma.

The Protein Data Bank website provides the crystal structure of TSP-1 searchable by 1LSL (Tan, K. et al., J Cell Biol., 2002, 159, 373-382); 2ES3 (Tan, K., et al., J Biol Chem., 2008, 283, 3932-3941); 1Z78 and 2ERF (Tan, K., et al., Structure, 2006, 14, 33-42); and 3R6B (Klenotic, P. A., et al., Protein Expr Purif., 2011, 80, 253-259); as well as the crystal structure of TSP-1 bound to various compounds searchable by 2OUH and 2OUJ (Tan, K., et al., J Biol Chem., 2008, 283, 3932-3941); and 1ZA4 (Tan, K., et al., Structure, 2006, 14, 33-42).

Representative TSP-1 Targeting Ligands are provided in FIG. 1.

### CD40 Ligand (CD40L)

In some embodiments, the Target Extracellular Protein is human CD40 ligand (CD40L) (UniProtKB—P29965 (CD40L_HUMAN)). CD40L is a cytokine that acts as a ligand to CD40/TNFRSF5. It costimulates T-cell proliferation and cytokine production. Its cross-linking on T-cells generates a costimulatory signal which enhances the production of IL4 and IL10 in conjunction with the TCR/CD3 ligation and CD28 costimulation. CD40L induces the activation of NF-kappa-B, as well as kinases MAPK8 and PAK2 in T-cells. It also induces tyrosine phosphorylation of isoform 3 of CD28. CD40L mediates B-cell proliferation in the absence of co-stimulus as well as IgE production in the presence of IL4, and is involved in immunoglobulin class switching.

The Protein Data Bank website provides the crystal structure of CD40L searchable by 1ALY (Karpusas, M., et al., Structure, 1995, 3, 1031-1039); as well as the crystal structure of CD40L bound to various compounds searchable by 3QD6 (An, H. J., et al., J Biol Chem., 2011, 286, 11226-11235); and 6BRB (Karnell, J. L., et al., Sci Transl Med., 2019, 11(489), 6584).

The expression of CD40L has been implicated in HIV-associated neurocognitive disorders and cardiovascular complications. Representative CD40L Targeting Ligands are provided in FIG. 1.

### Urokinase-Type Plasminogen Activator (UPA)

In some embodiments, the Target Extracellular Protein is human urokinase-type plasminogen activator (UPA) (UniProtKB—P00749 (UROK_HUMAN)). Urokinase-type plasminogen activator (uPA), is a serine protease present in the blood and in the extracellular matrix of many tissues. The primary physiological substrate of this enzyme is plasminogen, which is an inactive form (zymogen) of the serine protease plasmin. Activation of plasmin triggers a proteolytic cascade that, depending on the physiological environment, participates in thrombolysis or extracellular matrix degradation. This cascade had been involved in vascular diseases and cancer progression. Elevated expression levels of urokinase and several other components of the plasminogen activation system are found to be correlated with tumor malignancy.

The Protein Data Bank website provides the crystal structure of UPA bound to various compounds searchable by 5ZA7, 5ZAJ, 5ZA8, 5ZA9, 5ZAE, 5ZAF, 5ZAG, 5ZAH, and 5ZC5 (Buckley, B. J. et al., J Med Chem., 2018, 61, 8299-8320); 5LHP, 5LHQ, 5LHR, and 5LHS (Kromann-Hansen, T. et al., Sci Rep., 2017, 7, 3385-3385); 2VNT (Fish, P. V. et al. J Med Chem., 2007, 50, 2341); 1OWD, 1OWE, 1OWH, 1OWI, 1OWJ, and 1OWK (Wendt, M. D. et al., J Med Chem., 2004, 47, 303-324); 1SQA, 1SQO, and 1SQT (Wendt, M. D., et al., Bioorg Med Chem Lett., 2004, 14, 3063-3068); 1U6Q (Bruncko, M. et al., Bioorg Med Chem Lett., 2005, 15, 93-98) 3OX7, 3OY5 and 3OY6 (Jiang, L. G. et al., J Mol Biol., 2011, 412, 235-250); 4OS1, 4OS2, 4OS4, 4OS5, 4OS6 and 4OS7 (Chen, S. et al., Nat Chem., 2014, 6, 1009-1016); 3IG6 (West, C. W. et al., Bioorg Med Chem Lett., 2009, 19, 5712-5715); 4X0W and 4X1P (Jiang, L. et al., Int J Biochem Cell Biol., 2015, 62, 88-92); 4X1N, 4X1Q, 4X1R and 4X1S (Zhao, B. et al., PLoS One, 2014, 9, e115872-e115872); 5WXO and 5WXP (Jiang, L. et al., Biochim Biophys Acta., 2018, 1862, 2017-2023); 4MNV, 4MNW, 4MNX, and 4MNY (Chen, S., et al., Angew Chem Int Ed Engl., 2014, 53, 1602-1606); 4GLY (Chen, S., et al., J Am Chem Soc., 2013, 135, 6562-6569); 4JK5 and 4JK5 (Chen, S., et al., Chembiochem., 2013, 14, 1316-1322); 3QN7 (Angelini, A. et al., ACS Chem Biol., 2012, 7, 817-821); 2NWN (Zhao, G. et al., J Struct Biol., 2007, 160, 1-10); 6NMB (Wu, G. et al., Blood Adv., 2019, 3, 729-733); 1W0Z, 1W10, 1W11, 1W12, 1W13, and 1W14 (Zeslawska, E. et al., J Mol Biol., 2003, 328, 109); 4DVA (Jiang, L et al., Biochem J., 2013, 449, 161-166) 6A8G 6A8N (Wang, D. et al., J Med Chem., 2019, 62, 2172-2183); 2VIN, 2VIO, 2VIP, 2VIQ, 2VIV, and 2VIW (Frederickson, M. et al., J Med Chem., 2008, 51, 183); 1EJN (Speri, S., et al., Proc Natl Acad Sci USA, 2000, 97, 5113-5118); 3PB1 (Lin, Z. et al., J Biol Chem., 2011, 286, 7027-7032); 3U73 (Xu, X. et al., J Mol Biol., 2012, 416, 629-641); 1C₅W, 1C₅X, 1C₅Y and IC5Z (Katz, B. A., et al., Chem Biol., 2000, 7, 299-312); 5XG4 (Xue, G. et al., Food Funct., 2017, 8, 2437-2443); 5WXF (Jiang, L. et al., Biochim Biophys Acta., 2018, 1862, 2017-2023); 5WXS, 4ZKS, 5WXQ, 5WXT, 5YC6, 5YC7, 5Z1C, (Jiang, L. et al.); 4H42 (Yu, H. Y. et al.,); 6AG3 and 6AG9 (Buckley, B. et al); 3KGP, 3KHV, 3KID, 3M61, 3MHW, and 3MWI (Jiang, L. G. et al.,) 4ZKN, 4ZKO and 4ZKR (Jiang, L. et al.); 208T, 208U, 208W (Zhao, G. et al.,); and 4FU7, 4FU8, 4FU9, 4FUB, 4FUC, 4FUD, 4FUE, 4FUF, 4FUG, 4FUH, 4FUI, and 4FUJ (Kang, Y. N. et al.).

Representative UPA Targeting Ligands are provided in FIG. 1. Additional UPA Targeting Ligands are provided in, for example, J Med Chem 38: 1511-22 (1995), Bioorg Med Chem Lett 11: 2253-6 (2001), Bioorg Med Chem Lett 14: 3063-8 (2004), J Med Chem 52: 3159-65 (2009), CSAR 1: (2012), Bioorg Med Chem 22: 3187-203 (2014), J Med Chem 50: 2341-51 (2007), J Mol Biol 329: 93-120 (2003), Bioorg Med Chem Lett 2:1399-1404 (1992), J Med Chem 35: 4297-305 (1992), J Med Chem 35: 4150-9 (1992), J Med Chem 49: 5785-93 (2006), Bioorg Med Chem 23: 3696-704 (2015), Bioorg Med Chem Lett 10: 983-7 (2000), J Med Chem 49: 5785-93 (2006), each of which is incorporated by reference herein.

### Plasminogen Activator, Tissue Type (TPA)

In some embodiments, the Target Extracellular Protein is human plasminogen activator, tissue type (TPA) (UniProtKB—P00750 (TPA_HUMAN)). TPA converts the abundant, but inactive, zymogen plasminogen to plasmin by hydrolyzing a single Arg-Val bond in plasminogen. By controlling plasmin-mediated proteolysis, it plays an important role in tissue remodeling and degradation, in cell migration and many other physiopathological events. TPA plays a direct role in facilitating neuronal migration. PLA has been shown activated in various cancers including oral malignancy.

The Protein Data Bank website provides the crystal structure of TPA searchable by 1VR1 (Dekker, R. J. et al., J Mol Biol., 1999, 293, 613-627); as well as the crystal structure of TPA bound to various compounds searchable by 1RTF (Lamba, D. et al., J Mol Biol., 1996, 258, 117-135); 1ASH (Renatus, M. et al., J Biol Chem., 1997, 272, 21713-21719); and 1BDA (Renatus, M. et al., EMBO J., 1997, 16, 4797-4805).

Representative TPA Targeting Ligands are provided in FIG. 1. Additional TPA Targeting Ligands are provided in, for example, Bioorg Med Chem Lett 15: 4411-6 (2005), Bioorg Med Chem Lett 13: 2781-4 (2003), Bioorg Med Chem Lett 6: 2913-2918 (1996), J Med Chem 44: 2753-71 (2001), J Med Chem 41: 5445-56 (1999), Bioorg Med Chem Lett 12: 3183-6 (2002), U.S. patent Ser. No. 10/118,930, J Biol Chem 285: 7892-902 (2010), each of which is incorporated by reference herein.

### Plasminogen (PLG)

In some embodiments, the Target Extracellular Protein is human plasminogen (PLG) (UniProtKB—P00747 (PLMN_HUMAN)). PLG dissolves the fibrin of blood clots and acts as a proteolytic factor in a variety of other processes including embryonic development, tissue remodeling, tumor invasion, and inflammation. It activates the urokinase-type plasminogen activator, collagenases and several complement zymogens, such as C1 and C5. Its role in tissue remodeling and tumor invasion may be modulated by CSPG4.

The Protein Data Bank website provides the crystal structure of PLG searchable by 1DDJ (Wang, X. et al., J. Mol. Biol., 2000, 295, 903-914); and 4DUR and 4DUU (Law, R. H. P., et al., Cell Rep., 2012, 1, 185-190).

Representative PLG Targeting Ligands are provided in FIG. 1. Additional PLG Targeting Ligands are provided in, for example, J Med Chem 35: 4297-305 (1992), J Med Chem 38: 1511-22 (1995), J Med Chem 56: 820-31 (2013), U.S. Pat. Nos. 8,598,206, 8,921,319, J Med Chem 55: 1171-80 (2012), Bioorg Med Chem Lett 12: 3183-6 (2002), Bioorg Med Chem 23: 3696-704 (2015), Bioorg Med Chem Lett 13: 723-8 (2003), Bioorg Med Chem Lett 7: 331-336 (1997), each of which is incorporated by reference herein.

### Plasminogen Activator Inhibitor-1 (PAI-1)

In some embodiments, the Target Extracellular Protein is human plasminogen activator inhibitor 1 (PAI-1) (UniProtKB—P05121 (PAI1_HUMAN)). PAI-1 is a serine protease inhibitor, and a primary inhibitor of tissue-type plasminogen activator (PLAT) and urokinase-type plasminogen activator (PLAU). As PLAT inhibitor, it is required for fibrinolysis down-regulation and is responsible for the controlled degradation of blood clot. As PLAU inhibitor, it is involved in the regulation of cell adhesion and spreading, and acts as a regulator of cell migration, independently of its role as protease inhibitor. Overexpression of PAI-1 favors angiogenesis, metastasis, and poor prognosis in tumors, including, but not limited to, oral cancers and breast cancers.

The Protein Data Bank website provides the crystal structure of PAI-1 searchable by 3Q02 and 3Q03 (Jensen, J. K. et al., J Biol Chem., 2011, 286, 29709-29717); 1B3K (Sharp, A. M. et al., Structure, 1999, 7, 111-118); 1C₅G (Tucker, H. M. et al., Nat Struct Biol., 1995, 2, 442-445) 1DVM (Stout, T. J. et al., Biochemistry, 2000, 39, 8460-8469); and 3UT3 (Lin, Z. H. et al.,); as well as the crystal structure of PAI-1 bound to various compounds searchable by 4AQH (Fjellstrom, O. et al., J Biol Chem., 2013, 288, 873); 3R4L (Jankun, J. et al., Int J Mol Med., 2012, 29 61-64); 1A7C (Xue, Y., et al., Structure, 1998, 6, 627-636); 1OC0 (Zhou, A. et al., Nat Struct Biol., 2003, 10, 541); 6I8S (Vousden, K. A. et al., Sci Rep., 2019, 9, 1605-1605); 4G80 and 4G8R (Li, S. H. et al., Proc Natl Acad Sci USA, 2013, 110, E4941-E4949); 6GWQ, 6GWN and 6GWP (Sillen, M. et al., J Thromb Haemost, 2019); and 4IC0 (Hong, Z. B. et al.,).

Representative PAI-1 Targeting Ligands are provided in FIG. 1. Additional PAI-1 Targeting Ligands are provided in, for example, J Biol Chem 285: 7892-902 (2010), U.S. Pat. No. 9,120,744, Bioorg Med Chem Lett 13: 3361-5 (2003), Bioorg Med Chem Lett 12: 1063-6 (2002), Bioorg Med Chem Lett 13: 1705-8 (2003), Bioorg Med Chem Lett 11: 2589-92 (2001), U.S. Pat. No. 9,718,760, each of which is incorporated by reference herein.

### Placenta Growth Factor (PIGF)

In some embodiments, the Target Extracellular Protein is human placental growth factor (PGF) (UniProtKB—P49763 (PLGF_HUMAN)). PGF is growth factor active in angiogenesis and endothelial cell growth, stimulating their proliferation and migration. It binds to the receptor FLT1/VEGFR-1. Isoform PlGF-2 binds NRP1/neuropilin-1 and NRP2/neuropilin-2 in a heparin-dependent manner. PGF also promotes cell tumor growth, and has been implicated in age-related macular degeneration (AMD) and choroidal neovascularization (CNV).

The Protein Data Bank website provides the crystal structure of PIGF searchable by 1FZV (Iyer, S. et al., J Biol Chem., 2001, 276, 12153-12161); as well as the crystal structure of PIGF bound to various compounds searchable by 1RV6 (Christinger, H. W., J Biol Chem., 2004, 279, 10382-10388). Additionally, De Falco provides insight into the discovery and biological activity of placenta growth factor (De Falco, Exp Mol Med., 2012, 44, 1-9).

Representative PGF Targeting Ligands are provided in FIG. 1. Additional PGF Targeting Ligands are provided in, for example, J Med Chem 54: 1256-65 (2011), J Nat Prod 76: 29-35 (2013), each of which is incorporated by reference herein.

### Phospholipase A2, Group IB (PA21B)

In some embodiments, the Target Extracellular Protein is human phospholipase A2, Group IB (PA21B) (UniProtKB—P04054 (PA21B_HUMAN)). PA21B cleaves phospholipids preferentially at the sn-2 position, liberating free fatty acids and lysophospholipids. PA21B has been implicated in a number of diseases, including cardiovascular diseases, atherosclerosis, immune disorders and cancer.

The Protein Data Bank website provides the crystal structure of PA21B searchable by 3FVJ and 3FVI (Pan, Y. H. et al., Biochim. Biophys. Acta., 2010, 1804, 1443-1448).

Representative PA21B Targeting Ligands are provided in FIG. 1. Additional PA21B Targeting Ligands are provided in, for example, J Med Chem 39: 3636-58 (1996), Chembiochem 4: 181-5 (2003), J Med Chem 39: 5159-75 (1997), J Med Chem 51: 4708-14 (2008), each of which is incorporated by reference herein.

### Phospholipase A2, Group IIA (PA2GA)

In some embodiments, the Target Extracellular Protein is human phospholipase A2, Group IIA (PA2GA) (UniProtKB—P04054 (PA21B_HUMAN)). PA2GA catalyzes the calcium-dependent hydrolysis of the 2-acyl groups in 3-sn-phosphoglycerides. It is thought to participate in the regulation of phospholipid metabolism in biomembranes including eicosanoid biosynthesis. Independent of its catalytic activity, it also acts as a ligand for integrins. PA2GA Induces cell proliferation in an integrin-dependent manner. PA2GA has been implicated in a number of diseases, including cardiovascular diseases, atherosclerosis, immune disorders, and cancer.

The Protein Data Bank website provides the crystal structure of PA2GA bound to various compounds searchable by 2ARM and 1SV3 (Singh, N. et al., Proteins, 2006, 64, 89-100); 5G3M and 5G3N (Giordanetto, F., et al. ACS Med Chem Lett., 2016, 7, 884); 1KQU (Jansford, K. A., et al., Chembiochem., 2003, 4,181-185); and 1ZYX (Singh, N. et al.,). Additionally, Singh et al., provides insight into the crystal structure of the complexes of a group IIA phospholipase A2 with two natural anti-inflammatory agents, anisic acid, and atropine reveal a similar mode of binding (Singh, N. et al., Proteins, 2006, 64(1):89-100); and Kitadokoro et al also provides insight into the crystal structure of human secretory phospholipase A2-IIA complex with the potent indolizine inhibitor 120-1032 (Kitadokoro, K. et al., J Biochem., 1998, 123(4), 619-23).

Representative PA2GA Targeting Ligands are provided in FIG. 1. Additional PA2GA Targeting Ligands are provided in, for example, J Med Chem 48: 893-6 (2005), J Med Chem 39: 5159-75 (1997), each of which is incorporated by reference herein.

### Factor B

In some embodiments, the Target Extracellular Protein is human Complement factor B (UniProtKB—P00751 (CFAB_HUMAN)). Complement factor B, which is part of the alternate pathway of the complement system, is cleaved by factor D into 2 fragments: Ba and Bb. Bb, a serine protease, then combines with complement factor 3b to generate the C3 or C5 convertase. It has also been implicated in proliferation and differentiation of preactivated B-lymphocytes, rapid spreading of peripheral blood monocytes, stimulation of lymphocyte blastogenesis and lysis of erythrocytes. Ba inhibits the proliferation of preactivated B-lymphocytes.

The Protein Data Bank website provides the crystal structure of Complement Factor B searchable by 20K5 (Milder, F. J., et al., Nat Struct Mol Bio 2007, 14, 224-228); as well as the crystal structure of Complement factor B bound to various compounds searchable by 6QSW, 6QSX, and 6RAV (Schubart, A., et al., Proc Natl Acad Sci 2019, 116, 7926-7931); 6T8U, 6T8W, and 6T8V (Mainolfi, N., et al, J Med Chem 2020, 63, 5697-5722); and 7JTN (Xu, X., et al., J Immunol 2021, 206, doi:10.4049/jimmunol.2001260).

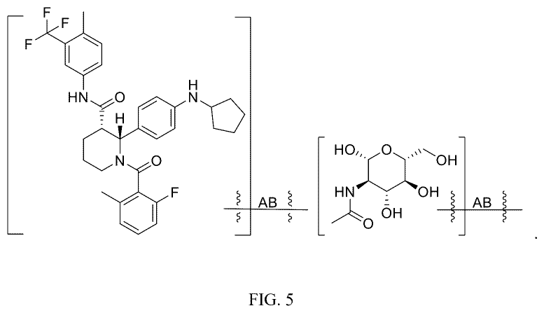

Representative Complement Factor B Targeting Ligands are provided in FIG. 5. Additional Complement Factor B Targeting Ligands are provided in, for example, U.S. Pat. Nos. 9,682,968B2, 9,475,806B2, 9,452,990B2, Proc Natl Acad Sci 116: 7926-7931 (2019), J Med Chem 52: 6042-6052 (2009), and J Med Chem 63: 5697-5722 (2020), each of which is incorporated by reference herein.

In certain embodiments the Extracellular Targeting Ligand is selected from:

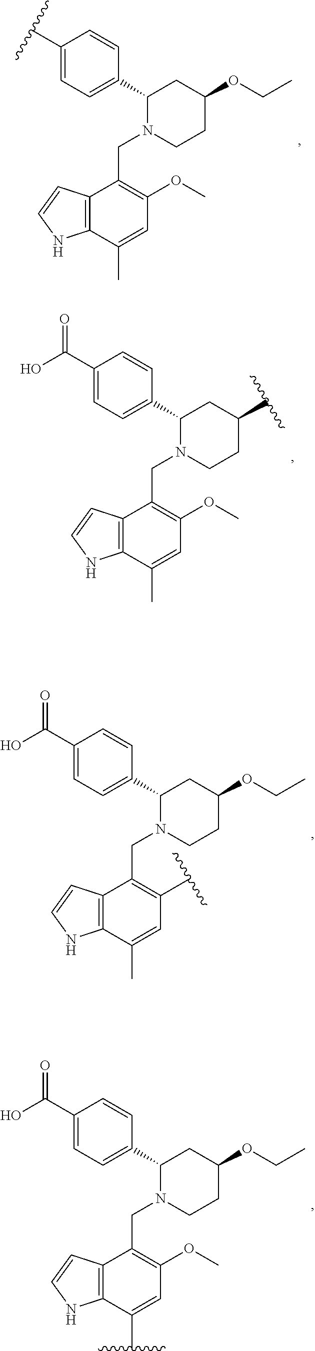 [SMILES: *c1ccc([C@@H]2C[C@@H](OCC)CCN2Cc2c(OC)cc(C)c3[nH]ccc23)cc1 ; *[C@H]1CCN(Cc2c(OC)cc(C)c3[nH]ccc23)[C@H](c2ccc(C(=O)O)cc2)C1 ; *c1cc(C)c2[nH]ccc2c1CN1CC[C@H](OCC)C[C@H]1c1ccc(C(=O)O)cc1 ; *c1cc(OC)c(CN2CC[C@H](OCC)C[C@H]2c2ccc(C(=O)O)cc2)c2cc[nH]c12]

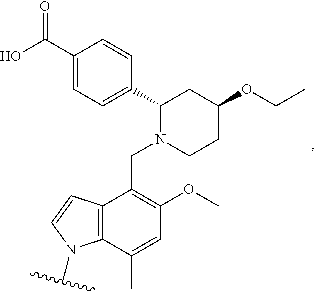 [SMILES: *n1ccc2c(CN3CC[C@H](OCC)C[C@H]3c3ccc(C(=O)O)cc3)c(OC)cc(C)c21]

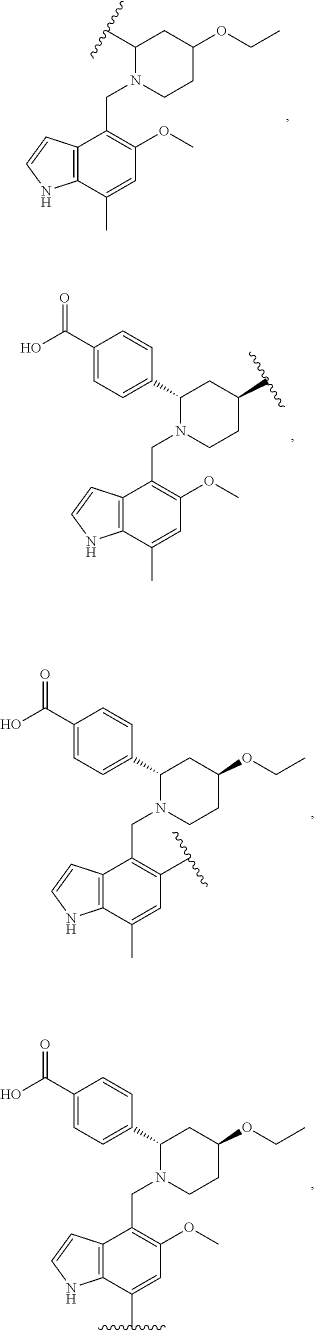 [SMILES: *C1CC(OCC)CCN1Cc1c(OC)cc(C)c2[nH]ccc12 ; *[C@H]1CCN(Cc2c(OC)cc(C)c3[nH]ccc23)[C@H](c2ccc(C(=O)O)cc2)C1 ; *c1cc(C)c2[nH]ccc2c1CN1CC[C@H](OCC)C[C@H]1c1ccc(C(=O)O)cc1 ; *c1cc(OC)c(CN2CC[C@H](OCC)C[C@H]2c2ccc(C(=O)O)cc2)c2cc[nH]c12]

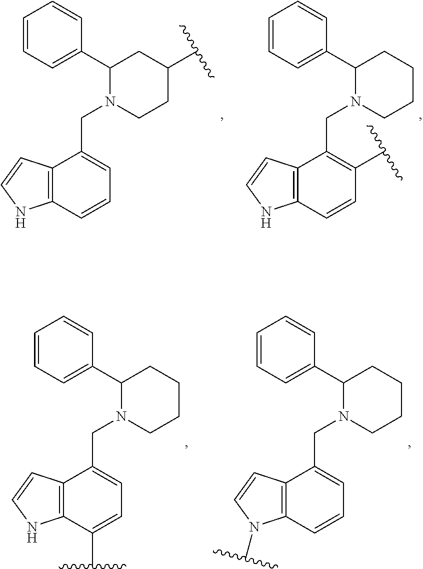 [SMILES: *C1CCN(Cc2cccc3[nH]ccc23)C(c2ccccc2)C1 ; *c1ccc2[nH]ccc2c1CN1CCCCC1c1ccccc1 ; *c1ccc(CN2CCCCC2c2ccccc2)c2cc[nH]c12 ; *n1ccc2c(CN3CCCCC3c3ccccc3)cccc21]

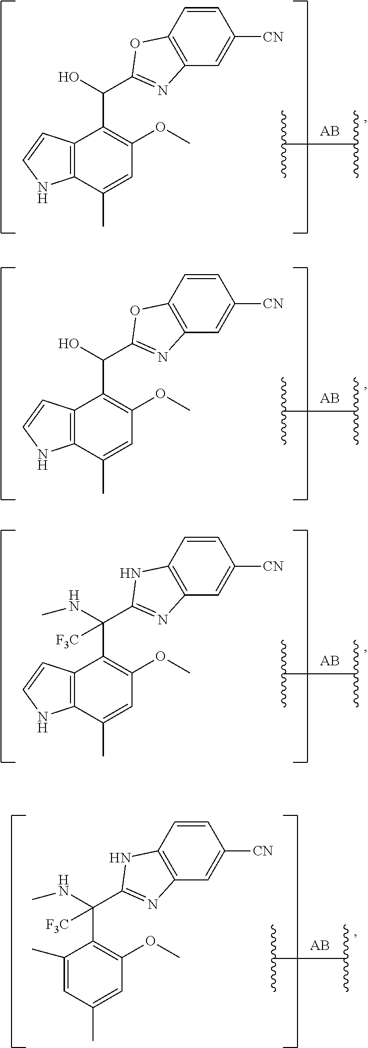

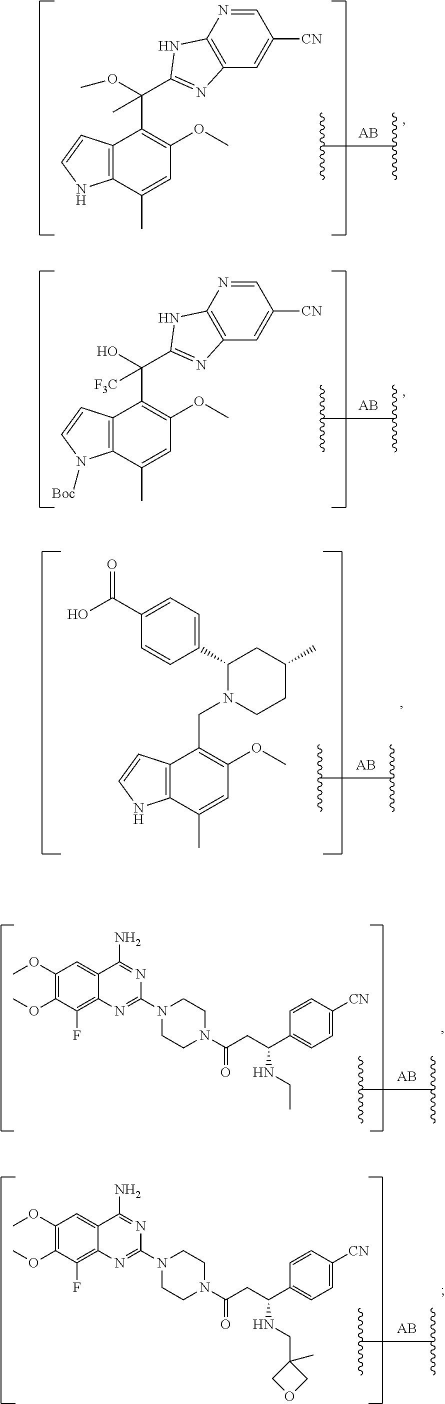

each of which is optionally substituted with 1, 2, 3, or 4 substituents independently selected from R²¹.

In certain embodiments the Factor B Targeting Ligand is selected from a ligand described in: Mainolfi, N. et. al. Discovery of 4-((2S,4S)-4-Ethoxy-1-((5-Methoxy-7-Methyl-1H-Indol-4-Yl)Methyl)Piperidin-2-Yl)Benzoic Acid (LNP023), a Factor B Inhibitor Specifically Designed To Be Applicable to Treating a Diverse Array of Complement Mediated Diseases. J. Med. Chem. 2020, 63 (11), 5697-5722; WO2020/016749; WO2018/005552; WO2013/192345; or WO2015009616.

In certain embodiments the factor B Targeting Ligand-linker is selected from:

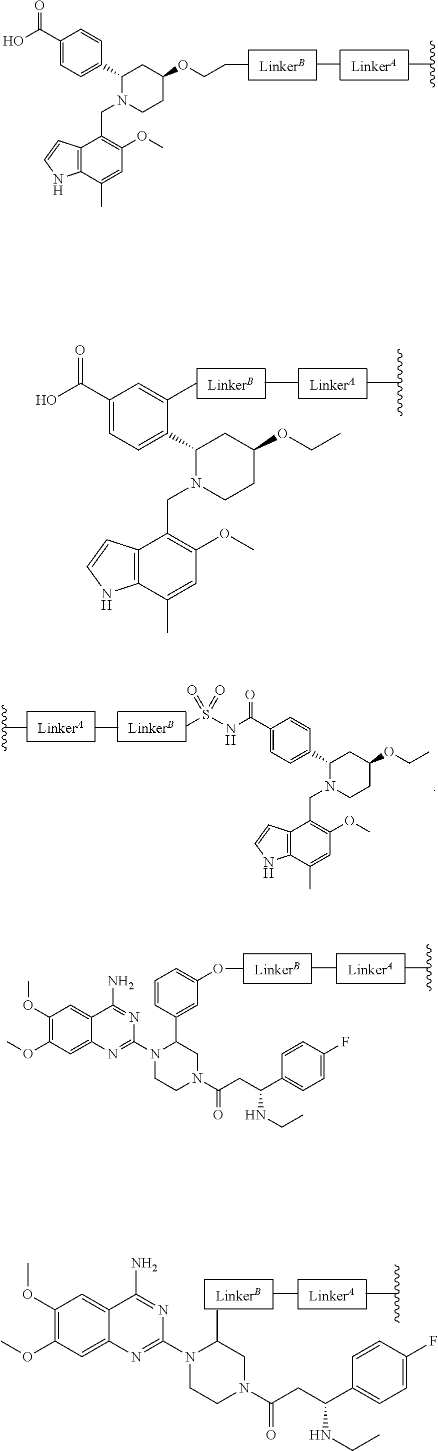

In certain embodiments the compound of the present invention is selected from the following compounds or a bi- or tri-dentate version thereof:

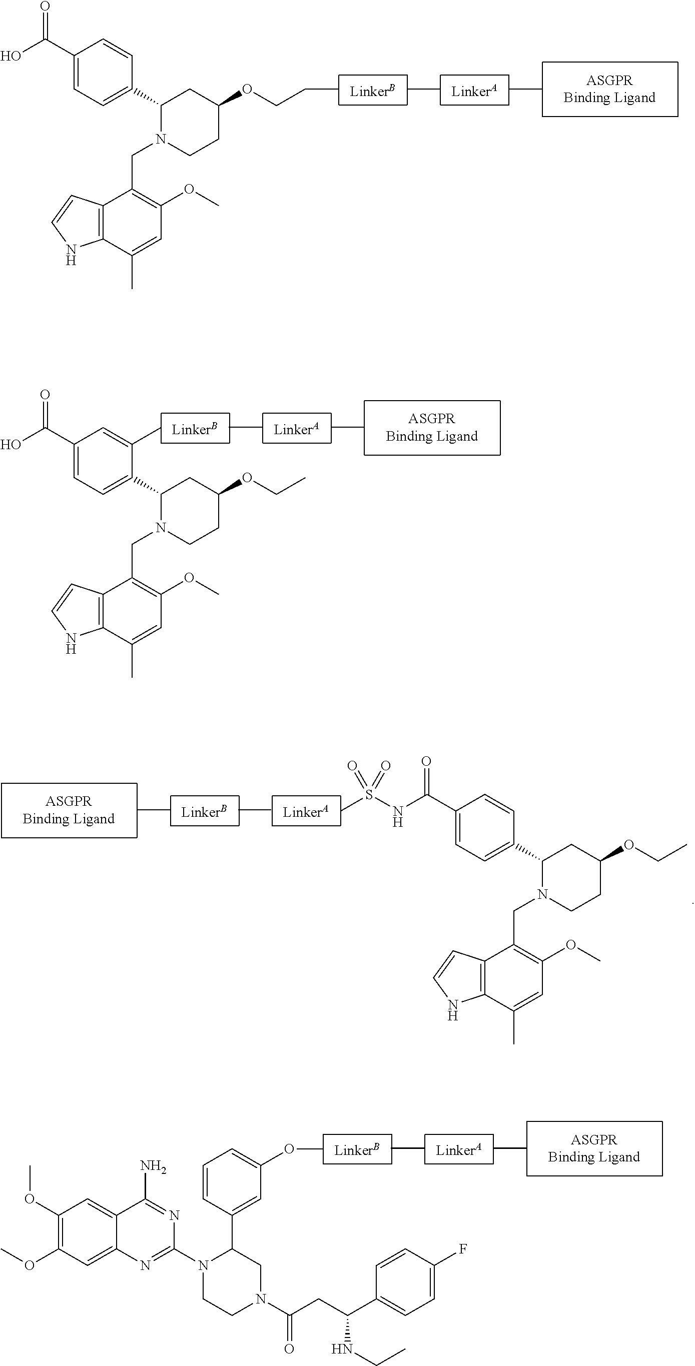

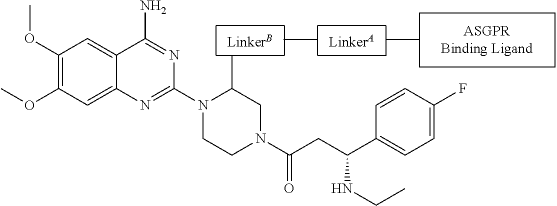

### Factor D

In some embodiments, the Target Extracellular Protein is human Complement factor D (UniProtKB—P00746 (CFAD_HUMAN)). Factor D cleaves factor B when the latter is complexed with factor C3b, activating the C3bbb complex, which then becomes the C3 convertase of the alternate pathway. Its function is homologous to that of C1s in the classical pathway.

The Protein Data Bank website provides the crystal structure of Complement factor D bound to various compounds searchable by 6FTZ, 6FUT, 6FUH, 6FUG, 6FUJ, and 6FUI (Vulpetti, A., et al., ACS Med Chem Lett 2018, 9, 490-495); 5TCA and 5TCC (Yang, C. Y., et al., ACS Med Chem Lett 2016, 7, 1092-1096); 5MT4 (Vulpetti, A., et al., J Med Chem 2017, 60, 1946-1958) 1DFP (Cole, L. B., et al., Acta Crystallogr D Biol Crystallogr 1997, 53, 143-150); 1DIC (Cole, L. B., et al., Acta Crystallogr D Biol Crystallogr 1998, 54, 711-717); 6QMR and 6QMT (Karki, R. G., et al., J Med Chem 2019, 62, 4656-4668).

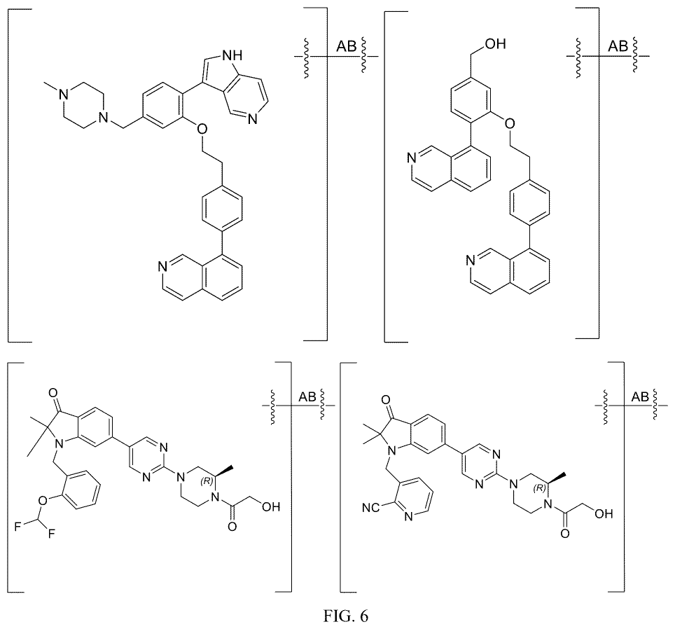

Representative Complement factor D Targeting Ligands are provided in FIG. 6. Additional Complement Factor D Targeting Ligands are provided in, for example, J Med Chem 60: 5717-5735 (2017), Nat Chem Biol 12: 1105-1110 (2016), U.S. Pat. Nos. 9,598,446B2, 9,643,986B2, 9,663,543B2, 9,695,205B2, 9,732,103B2, 9,732,104B2, 9,758,537B2, 9,796,741B2, 9,828,396B2, 10,000,516B2, 10,005,802B2, 10,011,612B2, 10,081,645B2, 10,087,203B2, 10,092,584B2, 10,100,072B2, 10,106,563B2, 10,138,225B2, 10,189,869B2, 10,253,053B2, 10,287,301B2, 10,301,336B2, 10,370,394B2, 10,385,097B2, 10,428,094B2, 10,428,095B2, 10,464,956B2, 10,550,140B2, 10,660,876B2, 10,662,175B2, 10,689,409B2, 10,807,952B2, 10,822,352B2, 9,464,081B2, and Hematological 102: 466-475 (2017), each of which is incorporated by reference herein.

In certain embodiments the Extracellular Targeting Ligand is selected from:

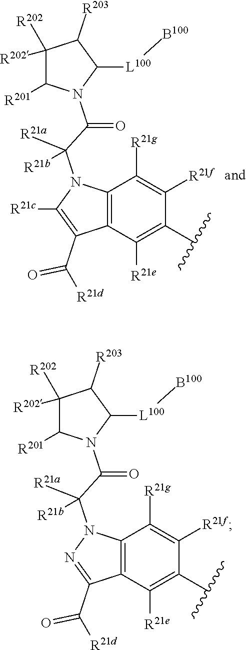

wherein:
R^{21a}, R^{21b}, R^{21c}, R^{21d}, R^{21e}, R^{21f}, and R^{21g} are independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR'S(O)R³, haloalkyl, aryl, heteroaryl, heterocyclyl, —SR³, —C(O)OR³, —C(O)NR⁶NR⁷, —OR³, and heterocycle;
R²⁰¹, R²⁰², R²⁰²′, and R²⁰³ are independently selected from hydrogen, halogen, hydroxyl, nitro, cyano, amino, C₁-C₆alkyl, C₂-C₆alkenyl, C₁-C₆alkoxy, C₂-C₆alkynyl, C₂-C₆alkanoyl, C₁-C₆thioalkyl, hydroxyC₁-C₆alkyl, aminoC₁-C₆alkyl, —C₀-C₄alkylNR⁹R¹⁰, —C(O)OR⁹, —OC(O)R⁹, —NR⁹C(O)R¹⁰, —C(O)NR⁹R¹⁰, —OC(O)NR⁹R¹⁰, —O(heteroaryl), —NR⁹C(O)OR¹⁰, C₁-C₂haloalkyl, —C₀-C₄alkyl(C₃-C₇cycloalkyl) and —O—C₀-C₄alkyl(C₃-C₇cycloalkyl), and C₁-C₂haloalkoxy, where R²⁰⁹ and R²¹⁰ are independently chosen at each occurrence from hydrogen, C₁-C₆alkyl, and (C₃-C₇cycloalkyl)C₀-C₄alkyl;
or R²⁰² and R^{202′} may be taken together to form a 3- to 6-membered spiro ring optionally substituted with 1 or more substituents independently chosen from halogen, hydroxyl, cyano, —COOH, C₁-C₄alkyl (including in particular methyl), C₂-C₄alkenyl, C₂-C₄alkynyl, C₁-C₄alkoxy, C₂-C₄alkanoyl, hydroxyC₁-C₄alkyl, (mono- and di-C₁-C₄alkylamino)C₀-C₄alkyl, —C₀-C₄alkyl(C₃-C₇cycloalkyl), —O—C₀-C₄alkyl(C₃-C₇cycloalkyl), C₁-C₂haloalkyl, and C₁-C₂haloalkoxy.
or R²⁰¹ and R²⁰² may be taken together to form a 3-membered carbocyclic ring, optionally substituted with 1, 2, or 3 substituents selected from R²¹.
or R²⁰¹ and R²⁰² may be taken together to form a 4- to 6-membered carbocyclic ring or a 4- to 6-membered heterocyclic ring containing 1 or 2 heteroatoms independently chosen from N, O, and S, optionally substituted with 1, 2, or 3 substituents selected from R²¹.
R²⁰² and R²⁰³ may be taken together to form a 3- to 6-membered carbocyclic ring or a 3- to 6-membered heterocyclic ring optionally substituted with 1, 2, or 3 substituents selected from R²¹.
L¹⁰⁰ is selected from

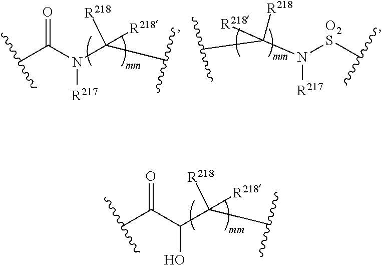

wherein R²¹⁷ is hydrogen or C₁-C₆alkyl and R²¹⁸ and R^{218′} are independently chosen from hydrogen, halogen, hydroxymethyl, and methyl; and mm is 0, 1, 2, or 3;
B¹⁰⁰ is a cycloalkyl, heterocycle group having 1, 2, 3, or 4 heteroatoms independently selected from N, O, and S, a C₂-C₆alkenyl, C₂-C₆alkynyl group, —(C₀-C₄alkyl)(aryl), —(C₀-C₄alkyl)(heteroaryl), or —(C₀-C₄alkyl)(biphenyl), each of which is optionally substituted with 1, 2, 3, or 4 substituents independently selected from R²¹.

In certain embodiments the Extracellular Targeting Ligand is selected from:

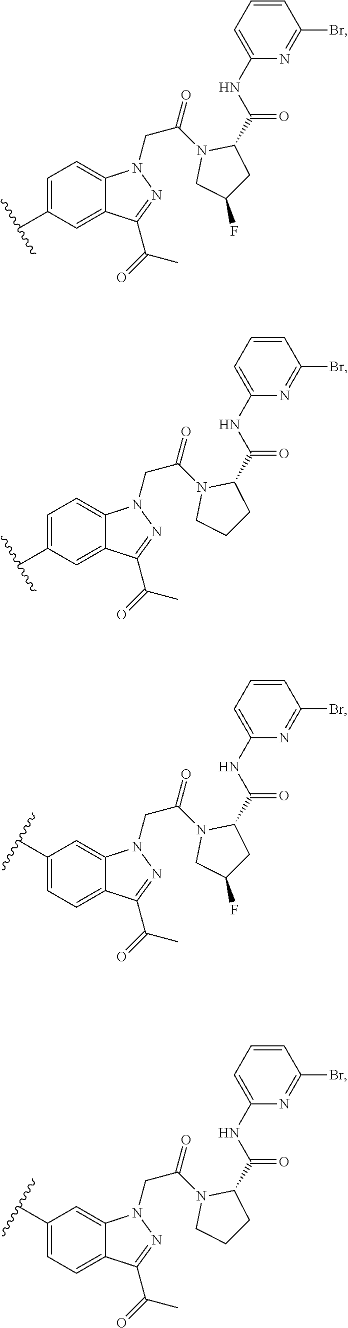 [SMILES: *c1ccc2c(c1)c(C(C)=O)nn2CC(=O)N1C[C@H](F)C[C@H]1C(=O)Nc1cccc(Br)n1 ; *c1ccc2c(c1)c(C(C)=O)nn2CC(=O)N1CCC[C@H]1C(=O)Nc1cccc(Br)n1 ; *c1ccc2c(C(C)=O)nn(CC(=O)N3C[C@H](F)C[C@H]3C(=O)Nc3cccc(Br)n3)c2c1 ; *c1ccc2c(C(C)=O)nn(CC(=O)N3CCC[C@H]3C(=O)Nc3cccc(Br)n3)c2c1]

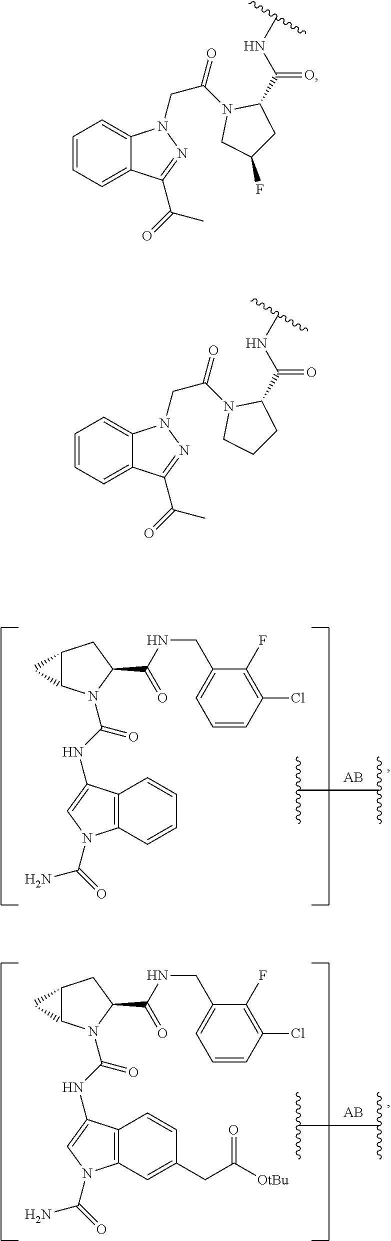

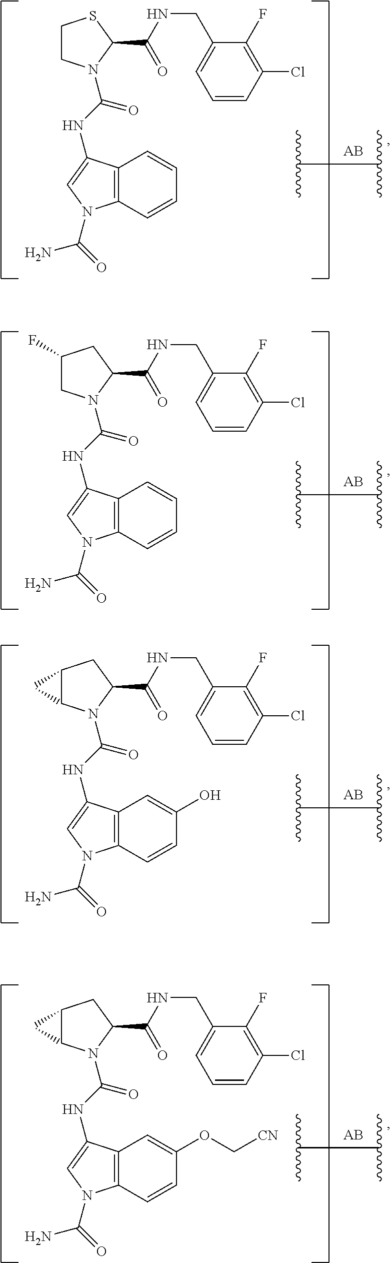

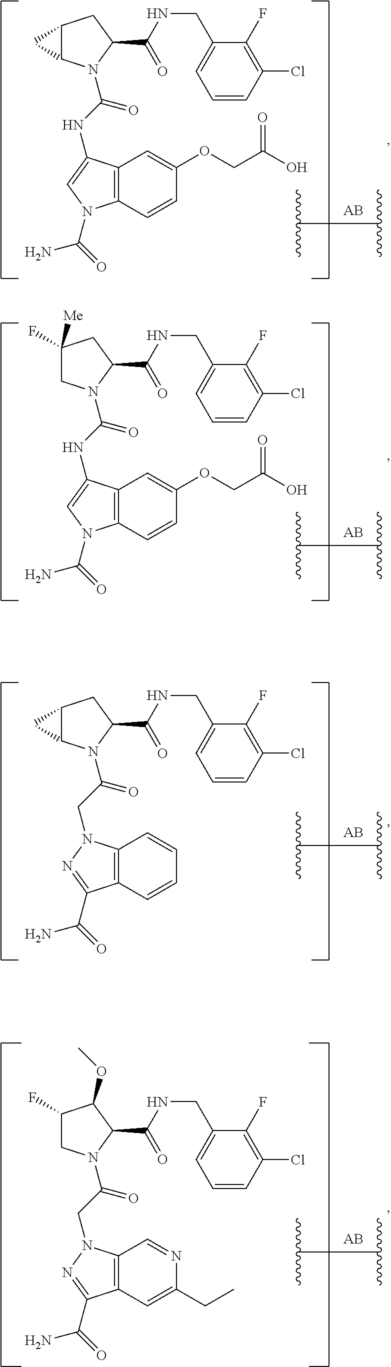

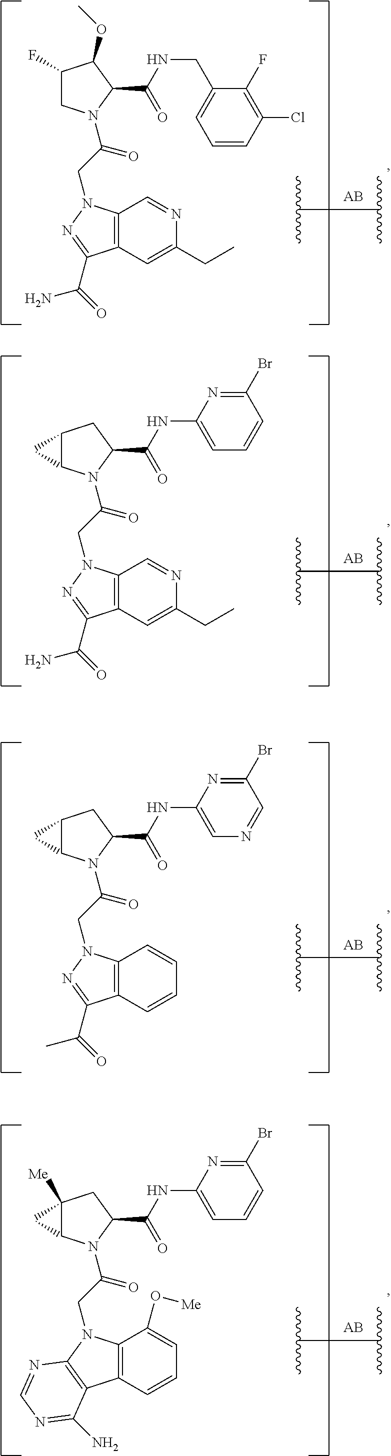

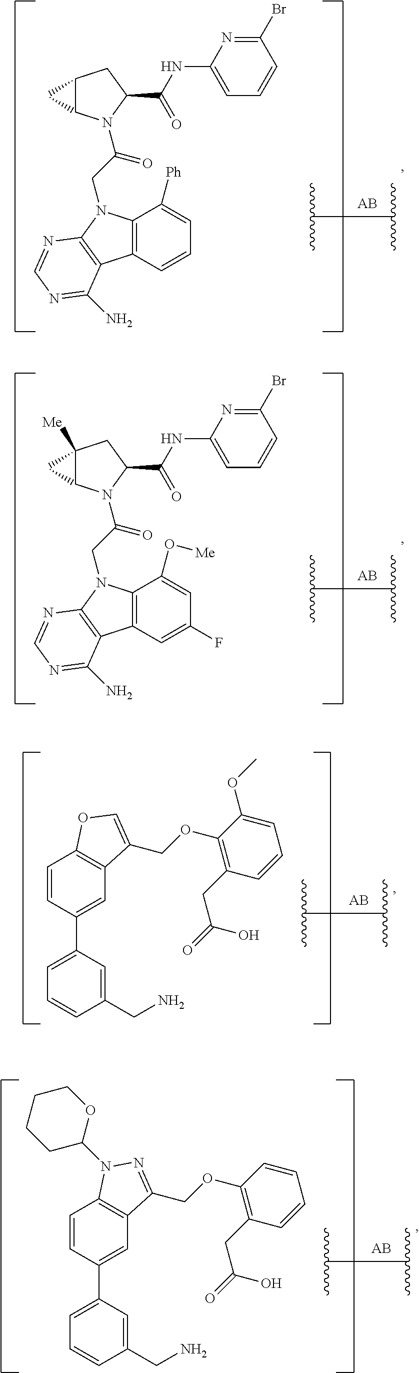

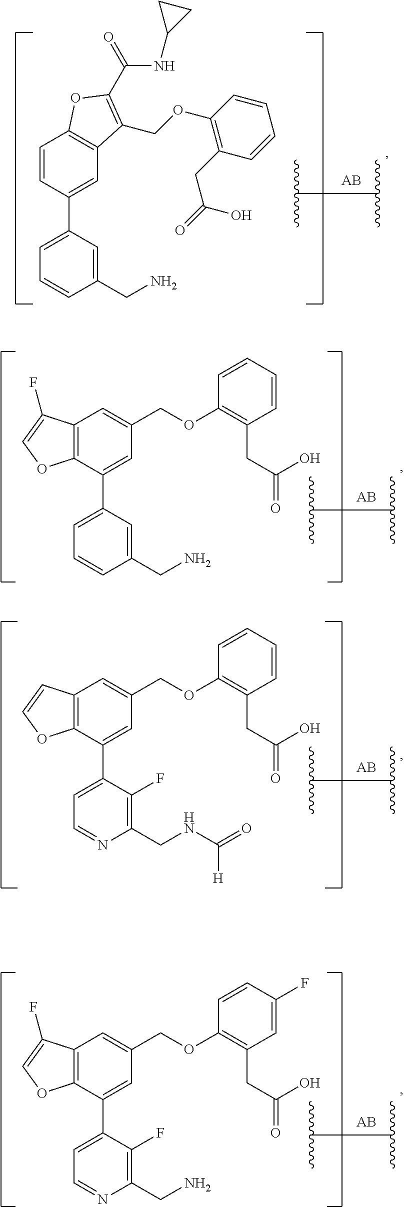

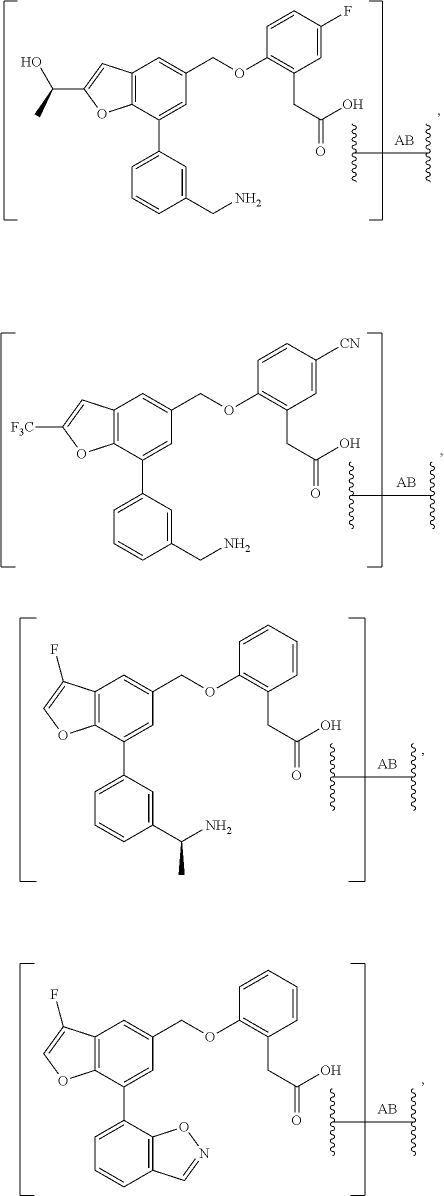

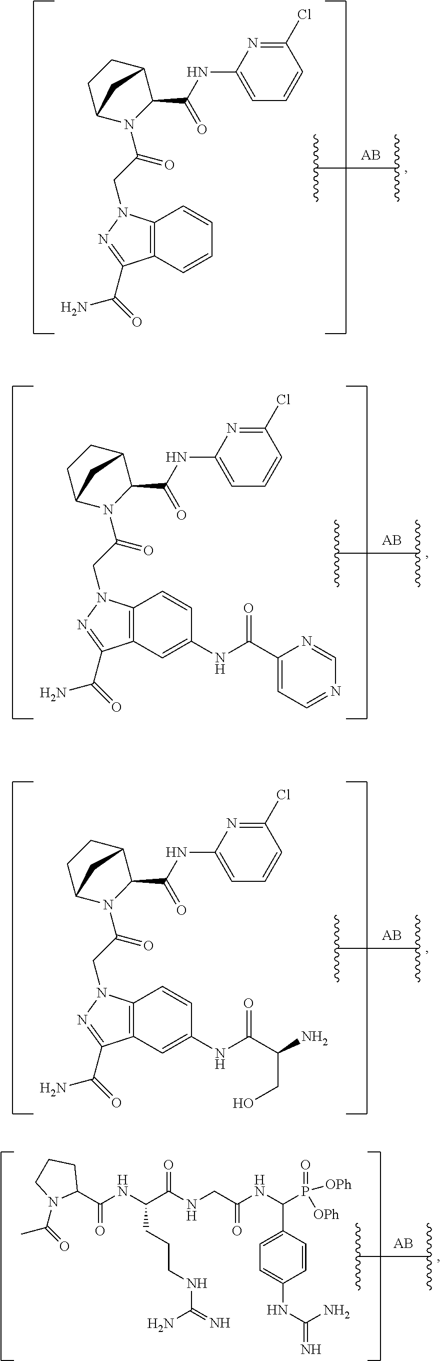

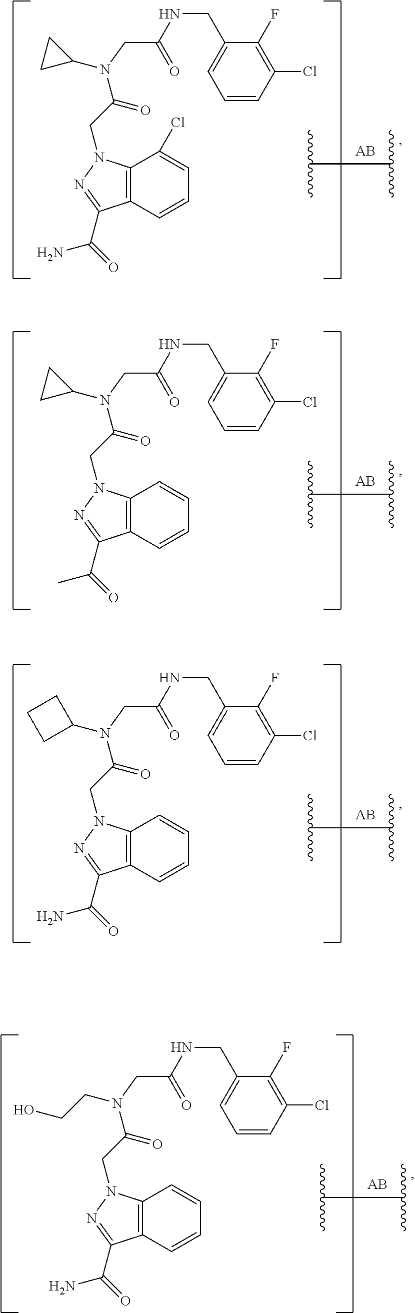

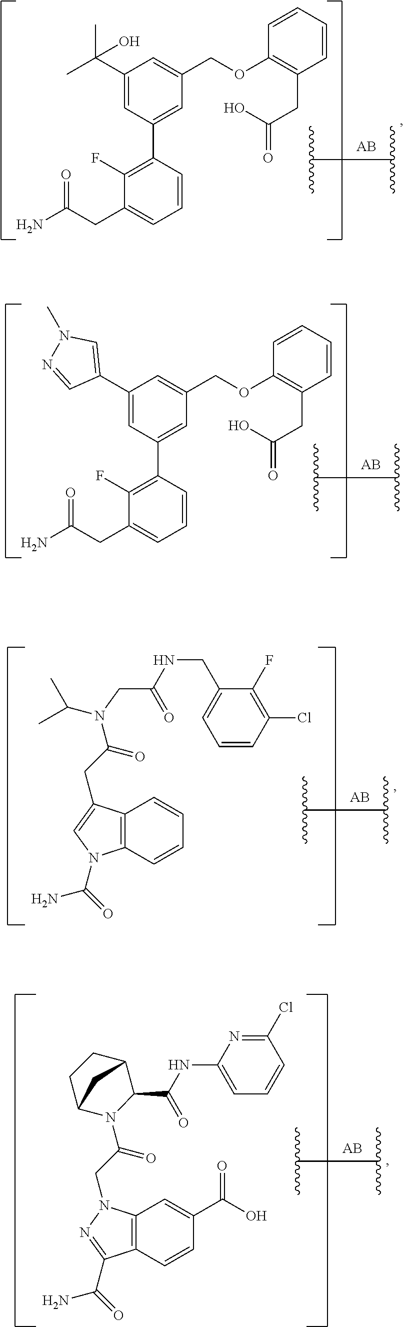

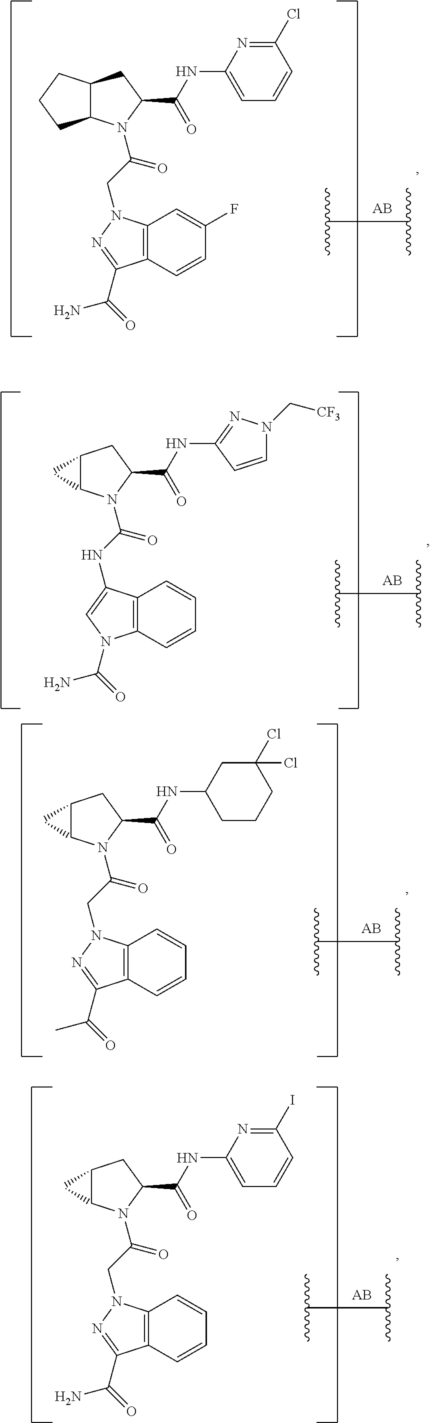

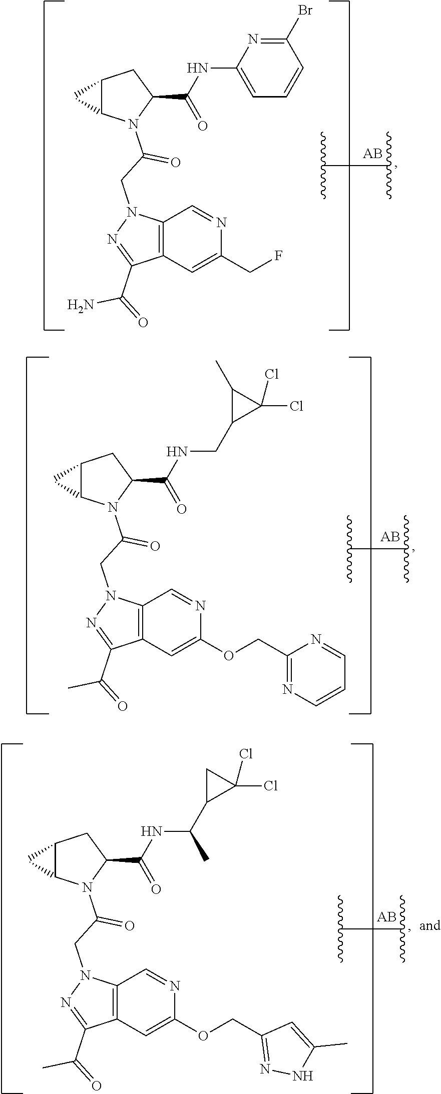

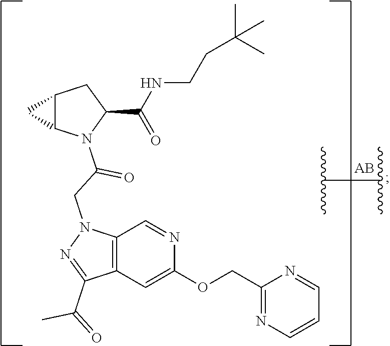

each of which is optionally substituted with 1, 2, 3, or 4 substituents independently selected from R²¹.

In certain embodiments the Factor D Targeting Ligand is selected from a ligand described in U.S. Pat. Nos. 9,796,74; 10,011,612; WO2018/160889; WO2019/195720; WO2019/057946; Karki, R. G. et al. Design, Synthesis, and Preclinical Characterization of Selective Factor D Inhibitors Targeting the Alternative Complement Pathway. J. Med. Chem. 2019, 62 (9), 4656-4668; or Belanger, D. B. et al.; WO2015/009977.

In certain embodiments the complement factor D targeting ligand-linker- is selected from:

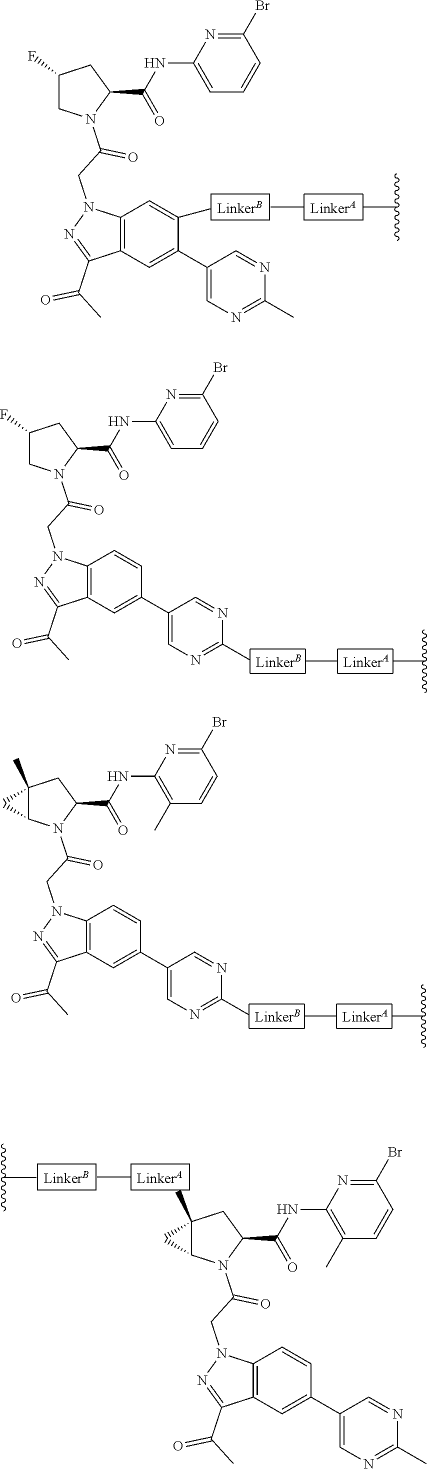

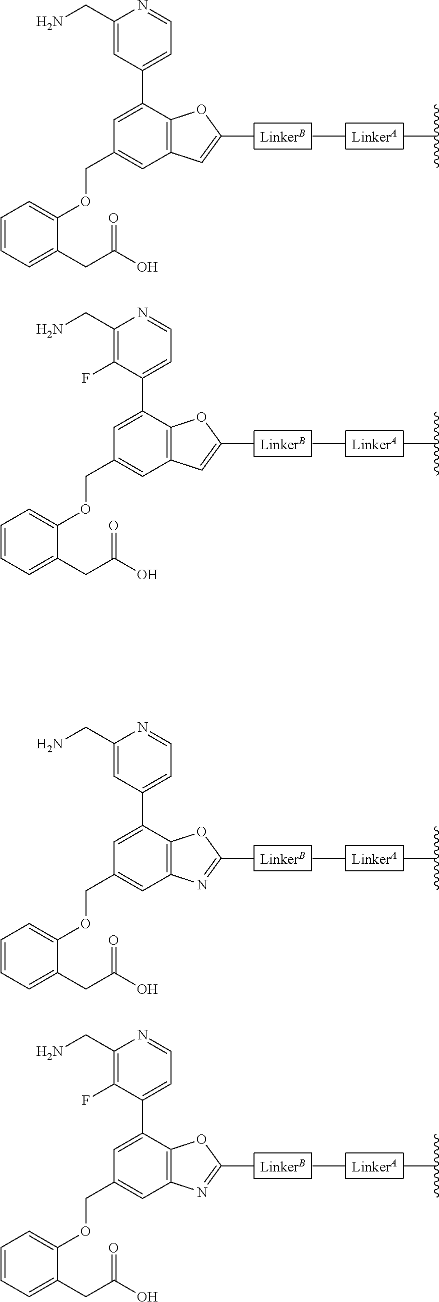

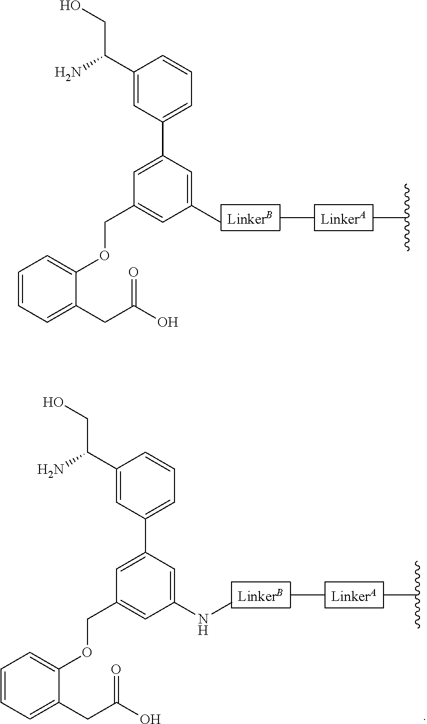

In certain embodiments the compound of the present invention is selected from the following compounds or a bi- or tri-dentate version thereof:

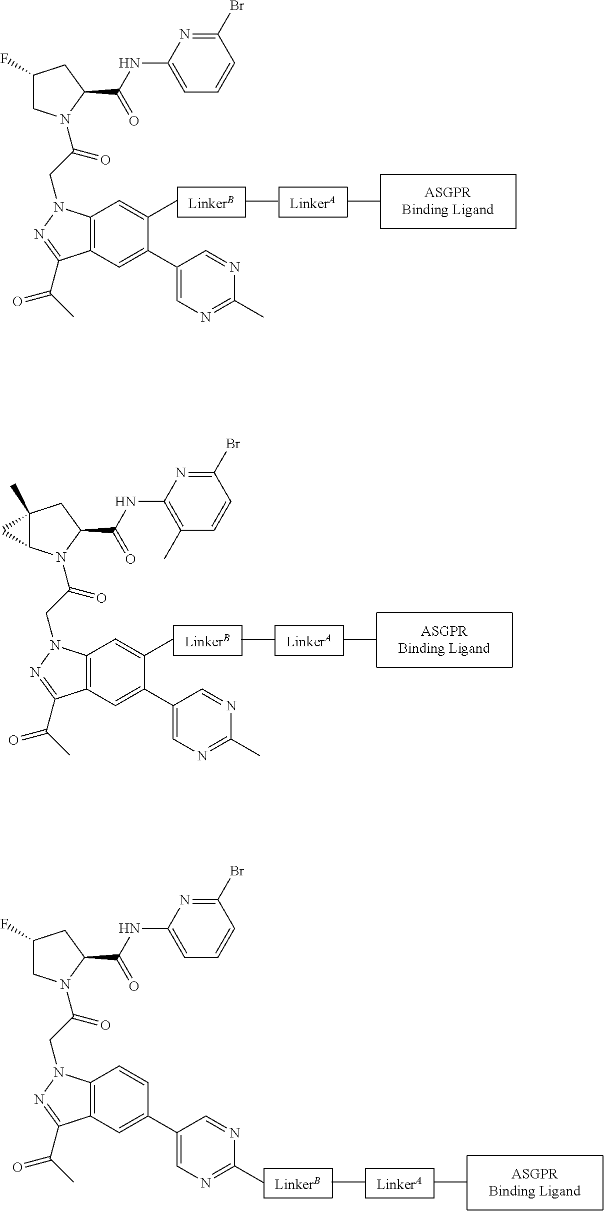

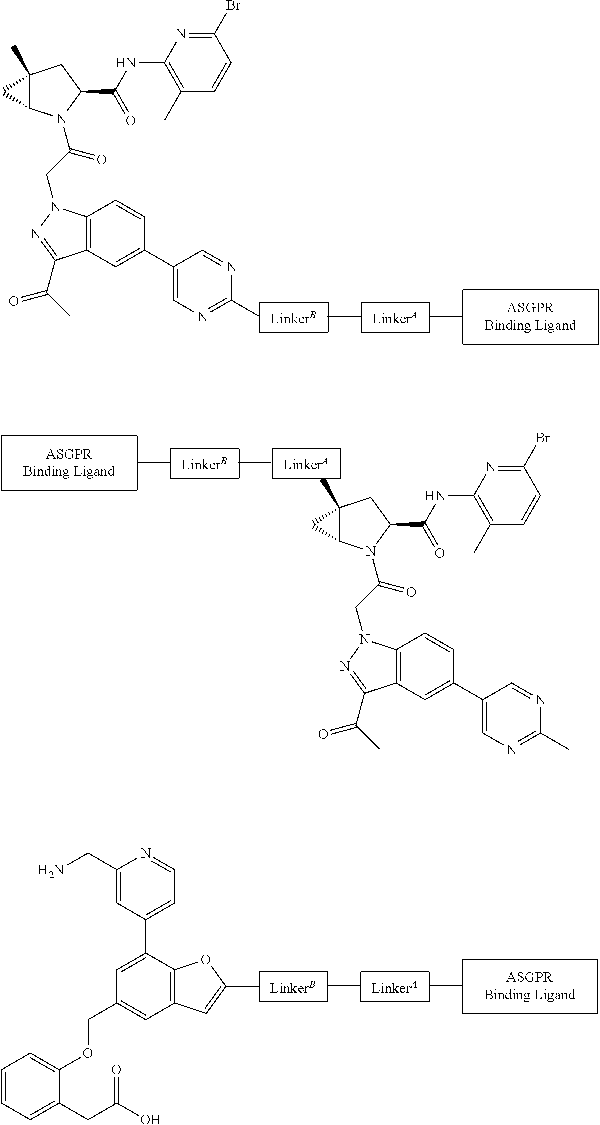

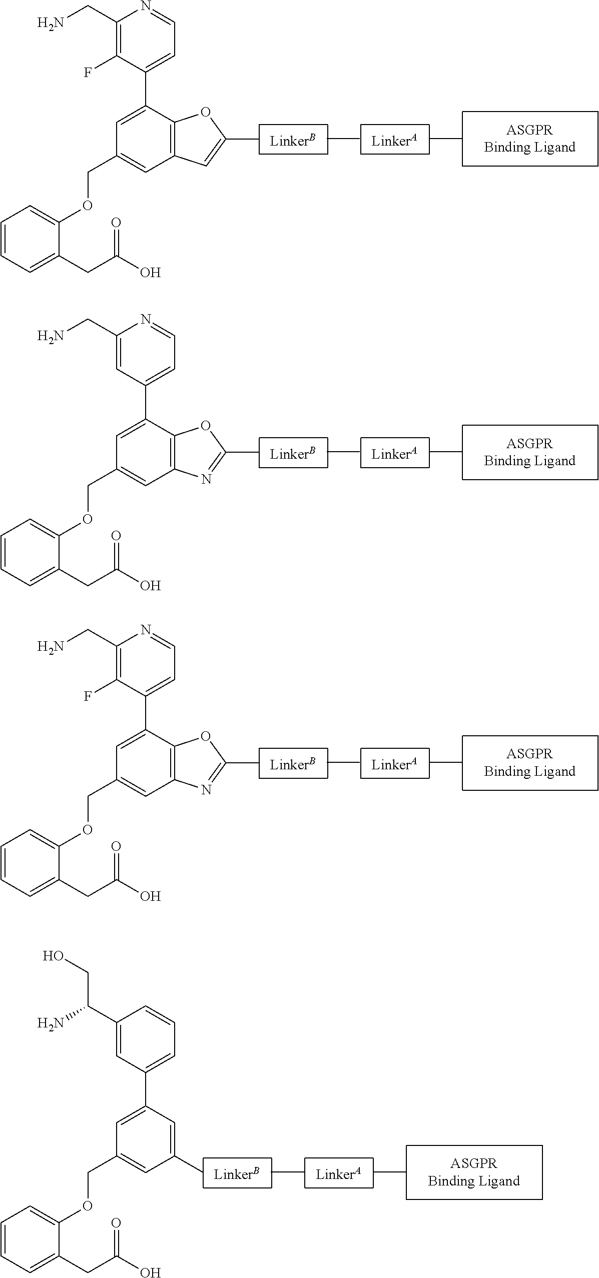

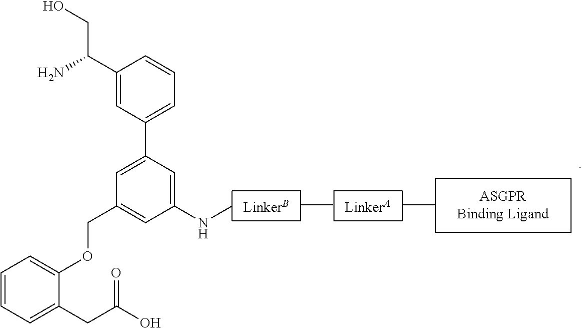

In certain embodiments the compound of the present invention is selected from the following compounds or a bi- or tri-dentate version thereof:

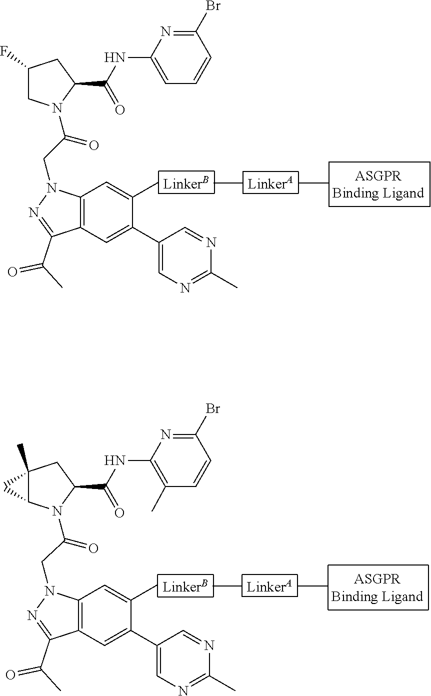

In certain embodiments the Factor D Targeting Ligand is selected from:

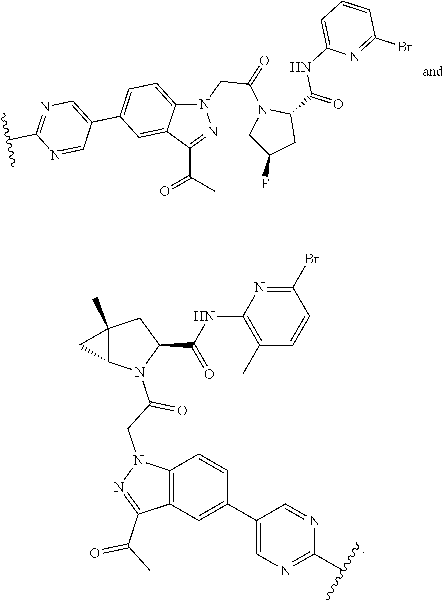

### Factor H

In some embodiments, the Target Extracellular Protein is human complement factor H (UniProtKB—P08603 (CFAH_HUMAN)). Complement factor H is a glycoprotein that plays an essential role in maintaining a well-balanced immune response by modulating complement activation. Acts as a soluble inhibitor of complement, where its binding to self-markers such as glycan structures prevents complement activation and amplification on cell surfaces. Complement factor H accelerates the decay of the complement alternative pathway (AP) C3 convertase C3bBb, thus preventing local formation of more C3b, the central player of the complement amplification loop. As a cofactor of the serine protease factor I, CFH also regulates proteolytic degradation of already-deposited C3b. In addition, it mediates several cellular responses through interaction with specific receptors. For example, CFH interacts with CR3/ITGAM receptor and thereby mediates the adhesion of human neutrophils to different pathogens. In turn, these pathogens are phagocytosed and destroyed.

The Protein Data Bank website provides the crystal structure of highly similar mutants of complement factor H searchable by 3KXV and 3KZJ (Bhattacharjee, A., et al., Mol Immunol 2010, 47, 1686-1691); as well as the crystal structure of wild type complement factor H bound to various compounds searchable by 2UWN (Prosser, B. E., et al., J Exp Med 2007, 204, 2277) 5WTB (Zhang, Y., et al., Biochem J 2017, 474, 1619-1631); 5032 and 5035 (Xue, X., et al., Nat Struct Mol Biol 2017, 24, 643-651); 40NT (Blaum, B. S., et al., Nat Chem Biol 2015, 11, 77-82); and 4ZH1 (Blaum, B. S., et al., Glycobiology 2016, 26, 532-539).

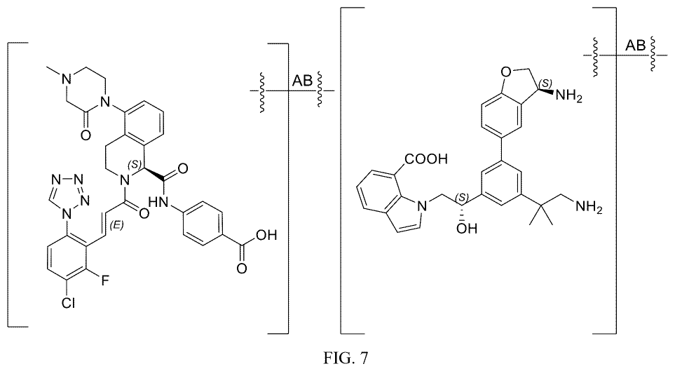

Representative complement factor H Targeting Ligands are provided in FIG. 7. Additional complement factor H Targeting Ligands are provided in, for example, J Immunol 182: 6394-6400 (2009), PLoS Pathogens 4: e1000250 (2008), PLoS Pathogens 6: e1001027 (2010), U.S. patent Ser. No. 10/865,238B1, U.S. Pat. No. 8,962,795B2, US patent application 20160317573A1, and US patent application 20190315842A1, each of which is incorporated by reference herein.

### Complement Component 5 (C5)

In some embodiments, the Target Extracellular Protein is human complement component 5 (C5) (UniProtKB—P01031 (CO5_HUMAN)). Activation of C5 by a C5 convertase initiates the spontaneous assembly of the late complement components, C₅-C₉, into the membrane attack complex. C5b has a transient binding site for C6. The C5b-C6 complex is the foundation upon which the lytic complex is assembled.

The Protein Data Bank website provides the crystal structure of Complement Component 5 searchable by 3CU7 (Fredslund, F., Nat Immunol 2008, 9, 753-760); as well as the crystal structure of Complement Component 5 bound to various compound searchable by 5I5K (Schatz-Jakobsen, J. A., et al, J Immunol 2016, 197, 337-344); 3PVM and 3PRX (Laursen, N. S., et al., EMBO J 2011, 30, 606-616); and 3KLS (Laursen, N. S., et al., Proc Natl Acad Sci 2010, 107, 3681-3686).

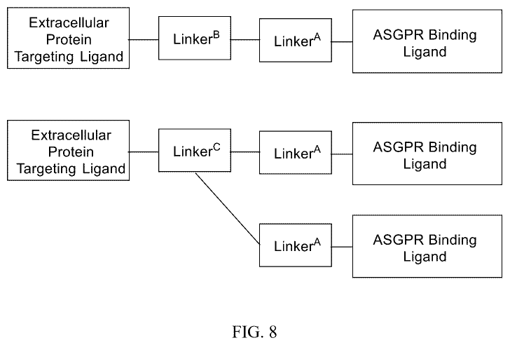

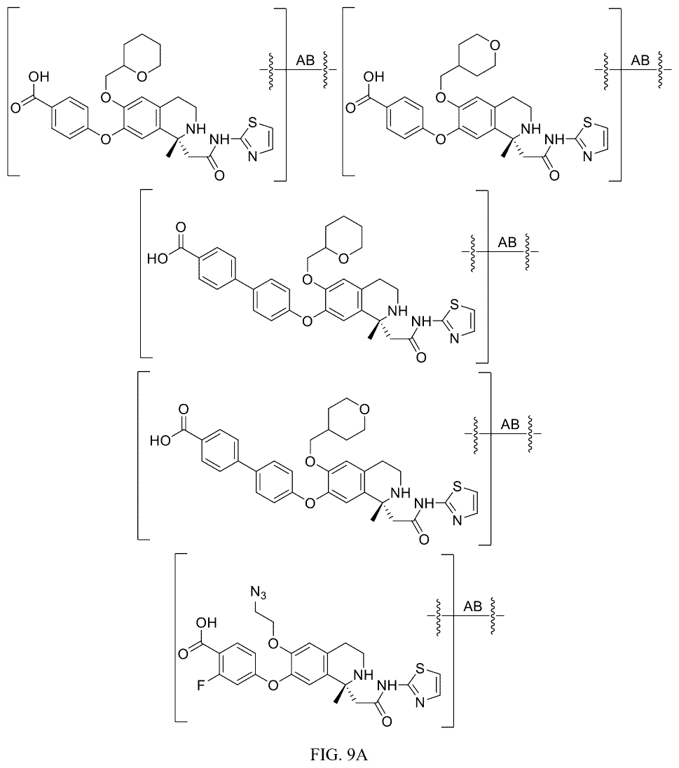

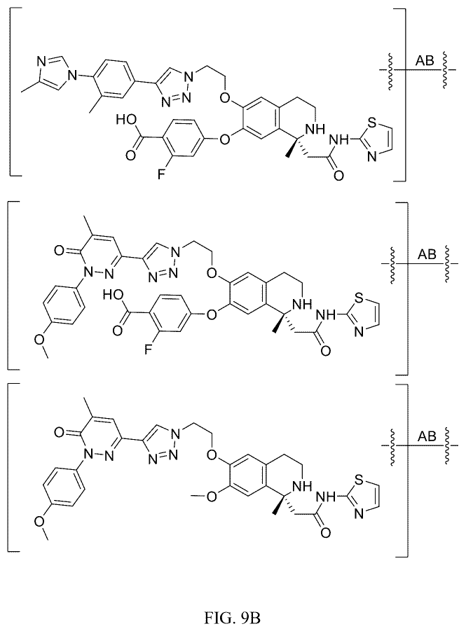

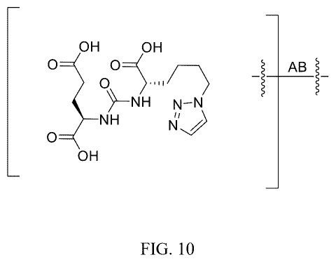

Representative Complement Component 5 Targeting Ligands are provided in FIG. 8. Additional Complement Component 5 Targeting Ligands are provided in, for example, J Immunol 197: 337-344 (2016), Ther Adv Hematol 10: 1-11 (2019), BioDrugs 34: 149-158 (2020), Blood 135: 884-885 (2020), US patent application 20170342139A1, and US patent application 20200095307A1, each of which is incorporated by reference herein.

In certain embodiments the Extracellular Targeting Ligand is selected from:

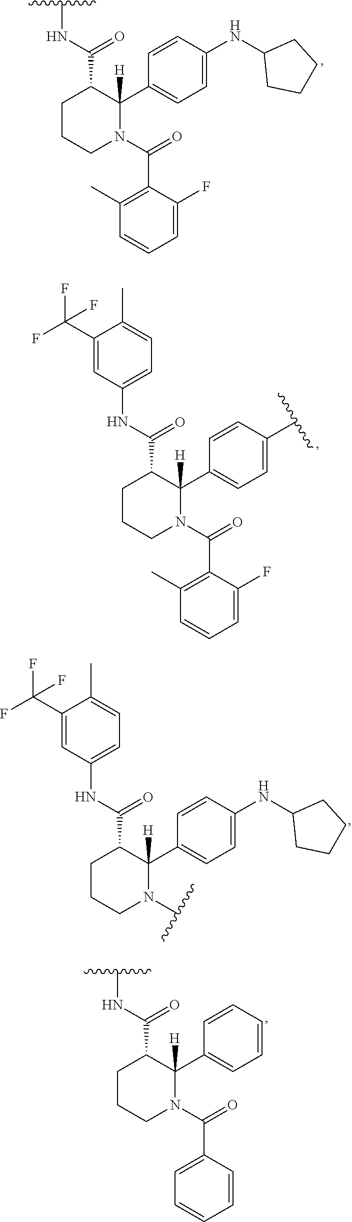

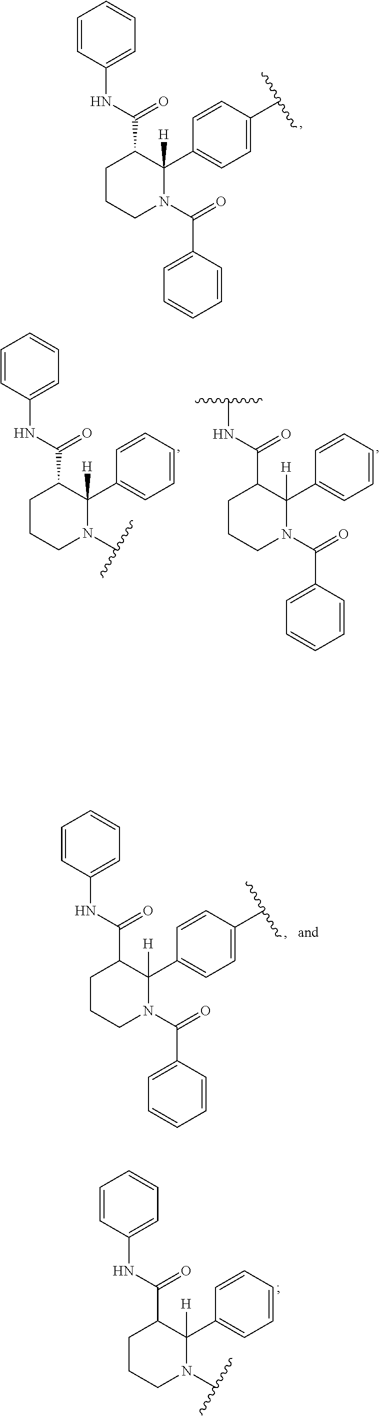 [SMILES: *c1ccc([C@H]2[C@@H](C(=O)Nc3ccccc3)CCCN2C(=O)c2ccccc2)cc1 ; *N1CCC[C@H](C(=O)Nc2ccccc2)[C@@H]1c1ccccc1 ; *NC(=O)C1CCCN(C(=O)c2ccccc2)C1c1ccccc1 ; *c1ccc(C2C(C(=O)Nc3ccccc3)CCCN2C(=O)c2ccccc2)cc1 ; *N1CCCC(C(=O)Nc2ccccc2)C1c1ccccc1]

each of which is optionally substituted with 1, 2, 3, or 4 substituents independently selected from R²¹.

In certain embodiments the complement C5 Targeting Ligand is selected from a ligand described in Jendza, K. et al. A Small-Molecule Inhibitor of C5 Complement Protein. Nat Chem Biol 2019, 15 (7), 666-668; or Zhang, M.; Yang, X.-Y.; Tang, W.; Groeneveld, T. W. L.; He, P.-L.; Zhu, F.-H.; Li, J.; Lu, W.; Blom, A. M.; Zuo, J.-P.; Nan, F.-J. Discovery and Structural Modification of 1-Phenyl-3-(1-Phenylethyl)Urea Derivatives as Inhibitors of Complement. ACS Med. Chem. Lett. 2012, 3 (4), 317-321.

In certain embodiments the C5 Targeting Ligand is selected from:

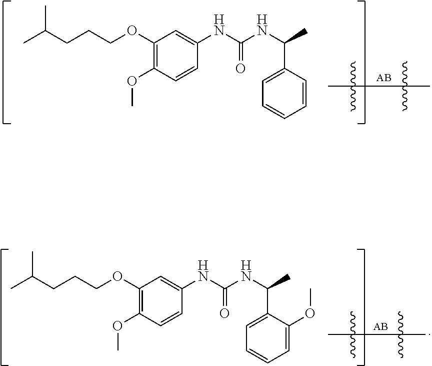

In certain embodiments the C5 Targeting Ligand is selected from:

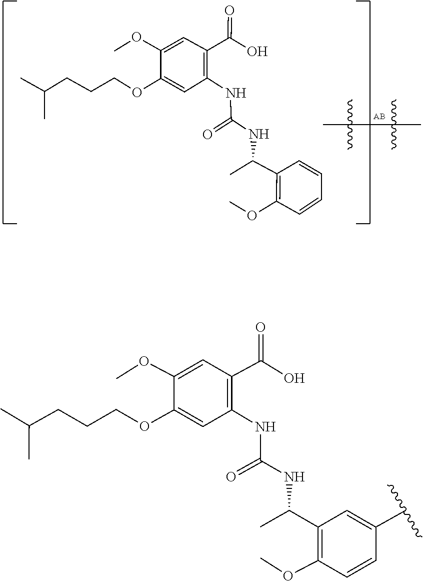

### Complement C1s

In certain embodiments the extracellular targeting ligand is a C1s Targeting Ligand.

In certain embodiments the complement C1s Targeting Ligand is selected from a ligand described in WO2020/198062 or U.S. Pat. No. 6,683,055.

In certain embodiments the compound of the present invention is selected from the following compounds or a bi- or tri-dentate version thereof:

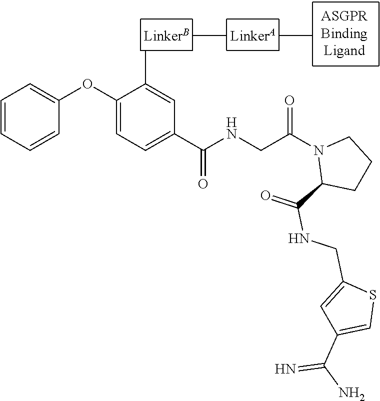

### MASP

In certain embodiments the extracellular targeting ligand is a MASP Targeting Ligand.

In certain embodiments the MASP Targeting Ligand is selected from a ligand described in Héja, D. et al. Monospecific Inhibitors Show That Both Mannan-Binding Lectin-Associated Serine Protease-1 (MASP-1) and -2 Are Essential for Lectin Pathway Activation and Reveal Structural Plasticity of MASP-2. Journal of Biological Chemistry 2012, 287 (24), 20290-20300; Dobó, J.; Kocsis, A.; Gil, P. Be on Target: Strategies of Targeting Alternative and Lectin Pathway Components in Complement-Mediated Diseases. Front. Immunol. 2018, 9, 1851; or WO 2014/144542.

In certain embodiments the MSAP-1 Targeting Ligand is SGMI-1 peptide, linked through the N- or C-terminus.

In certain embodiments the MSAP-1 Targeting Ligand is SGMI-2 peptide, linked through the N- or C-terminus.

In certain embodiments the MSAP-1 Targeting Ligand is TFMI-3 peptide, linked through the N- or C-terminus.

### Factor XIa

In certain embodiments the extracellular targeting ligand is a factor XIa Targeting Ligand.

In certain embodiments the factor XIa Targeting Ligand is selected from a ligand described in: Lorthiois, E. et al. Structure-Based Design and Preclinical Characterization of Selective and Orally Bioavailable Factor XIa Inhibitors: Demonstrating the Power of an Integrated S1 Protease Family Approach. J. Med. Chem. 2020, 63 (15), 8088-8113.

In certain embodiments the factor XIa Targeting Ligand is selected from a ligand described in: Quan, M. L. et al. Factor XIa Inhibitors as New Anticoagulants. J. Med. Chem. 2018, 61 (17) 7425-7447.

In certain embodiments the factor XIa Targeting Ligand is selected from a ligand described in: Yang, W. et al. Discovery of a High Affinity, Orally Bioavailable Macrocyclic FXIa Inhibitor with Antithrombotic Activity in Preclinical Species. J. Med. Chem. 2020, 63 (13), 7226-7242.

In certain embodiments the factor XIa Targeting Ligand-Linker is:

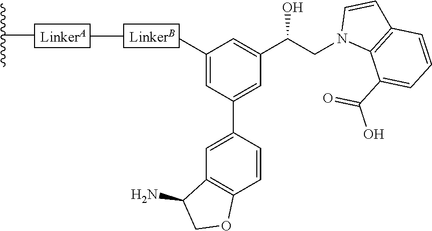

In certain embodiments the compound of the present invention is selected from the following compounds or a bi- or tri-dentate version thereof:

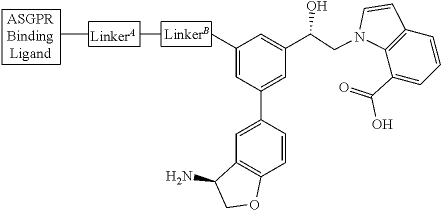

In certain embodiments the factor Xia Targeting Ligand is selected where an anchor bond is placed at any suitable location with or without functionalization.

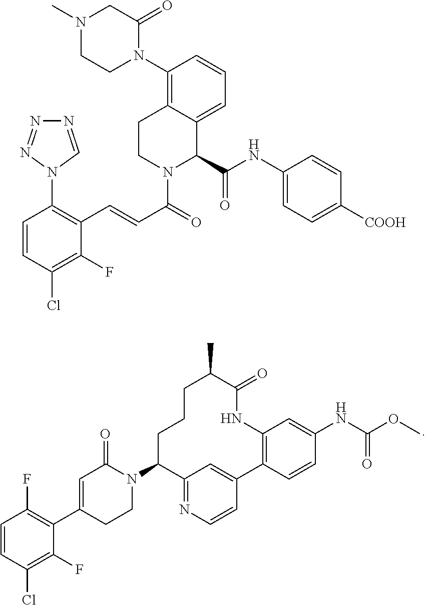 [SMILES: CN1CCN(c2cccc3c2CCN(C(=O)/C=C/c2c(-n4cnnn4)ccc(Cl)c2F)[C@@H]3C(=O)Nc2ccc(C(=O)O)cc2)C(=O)C1 ; COC(=O)Nc1ccc2c(c1)NC(=O)[C@H](C)CCC[C@H](N1CCC(c3c(F)ccc(Cl)c3F)=CC1=O)c1cc-2ccn1]

In certain embodiments the Factor XIa Targeting Ligand is selected from:

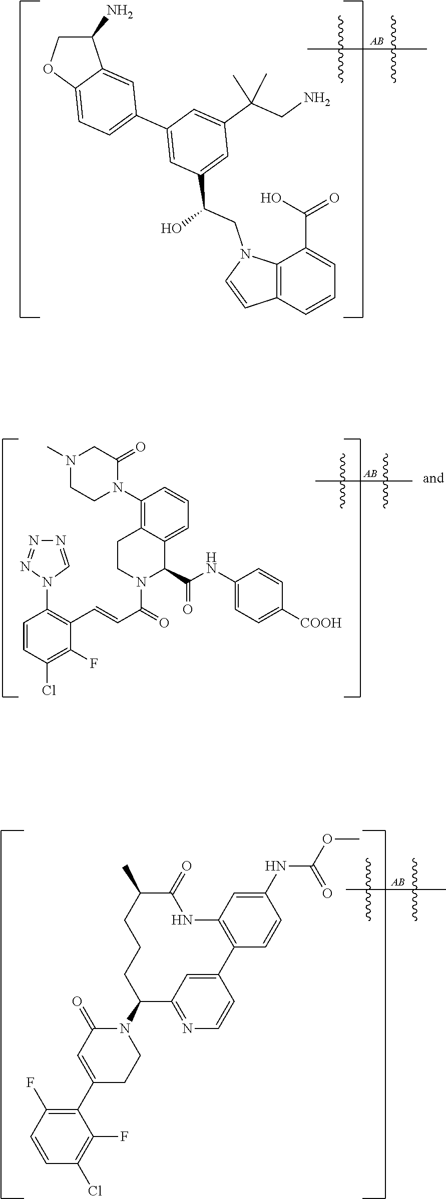

### Immunoglobulin Degradation

Immunoglobulins, for example IgG, can cause, modulate, or amplify diseases in vivo, such as abnormal cellular proliferation such as tumors and cancer, autoimmune disorders, inflammation, and aging-related diseases. For example, immunoglobulins bind to cell surface receptors, often initiating aberrant signaling in multiple diseases such as cancer and inflammation.

The immunoglobulin degraders described herein or their pharmaceutically acceptable salt and/or pharmaceutically acceptable compositions thereof can be used to treat a disorder which is mediated by an immunoglobulin that binds to the Immunoglobulin Targeting Ligand. The described degraders are capable of targeting immunoglobulins that mediate pathological disorders for lysosomal degradation. The selected immunoglobulin may modulate a disorder in a human via a mechanism of action such as modification of a biological pathway, pathogenic signaling, or modulation of a signal cascade or cellular entry. The immunoglobulin is recruited with an Immunoglobulin Targeting Ligand, which is a ligand for the immunoglobulin.

Accordingly, in some embodiments, a method to treat a host with a disorder mediated by an immunoglobulin is provided that includes administering an effective amount of a degrader targeting the immunoglobulin or its pharmaceutically acceptable salt described herein to the host, typically a human, optionally in a pharmaceutically acceptable composition.

The immunoglobulin can be either the normal form of the protein or an aberrant form. For example, the immunoglobulin can be a mutant protein, or a protein, for example, where a partial, or full, gain-of-function or loss-of-function is encoded by nucleotide polymorphisms.

Targeting specific immunoglobulins is accomplished by the present invention through the use of specific Immunoglobulin Targeting Ligands. The target immunoglobulins of the current invention may include, but are not limited to, immunoglobulin G (IgG), immunoglobulin A (IgA), and immunoglobulin E (IgE). These immunoglobulins mediate a range of diseases that can be treated with an effective amount of the disclosed ASGPR-binding Immunoglobulin Degraders described herein.

In certain embodiments, the Immunoglobulin Targeting Ligand comprises an antibody binding moiety described in WO 2019/023501, incorporated by reference herein.

### Immunoglobulin A (IgA)

Aberrant expression of immunoglobulin A (IgA) mediates a range of autoimmune and immune-mediated disorders, including IgA nephropathy (also known as Berger's disease), celiac disease, Crohn's disease, Henoch-Schonlein purpura (HSP) (also known as IgA vasculitis), IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), linear IgA bullous dermatosis, rheumatoid arthritis, ulcerative colitis, and primary glomerulonephritis, among others.

Specific degradation of IgA can be accomplished through the use of an IgA-specific Immunoglobulin Targeting Ligand. In certain embodiments, the Immunoglobulin Targeting Ligand used is an Opt peptide. Variations and derivatives of the IgA-specific Opt peptide suitable for use as IgA-specific Immunoglobulin Targeting Ligands are described in Hatanaka et al. Journal of Biological Chemistry, 287(51) 43126-43136. In certain embodiments, the IgA-specific Immunoglobulin Targeting Ligand is Opt-1. In certain embodiments, the IgA-specific Immunoglobulin Targeting Ligand is Opt-2. In certain embodiments, the IgA-specific Immunoglobulin Targeting Ligand is Opt-3.

In certain embodiments the Immunoglobulin Targeting Ligand is:

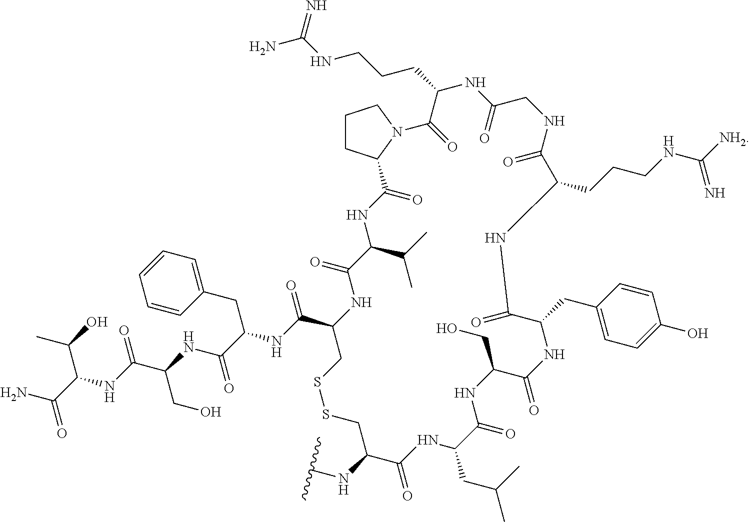 [SMILES: *N[C@H]1CSSC[C@@H](C(=O)N[C@@H](Cc2ccccc2)C(=O)N[C@@H](CO)C(=O)N[C@H](C(N)=O)[C@@H](C)O)NC(=O)[C@H](C(C)C)NC(=O)[C@@H]2CCCN2C(=O)[C@H](CCCNC(=N)N)NC(=O)CNC(=O)[C@H](CCCNC(=N)N)NC(=O)[C@H](Cc2ccc(O)cc2)NC(=O)[C@H](CO)NC(=O)[C@H](CC(C)C)NC1=O]

The Protein Data Bank website provides the crystal structure of IgA, as well as the crystal structure of IgA bound to various compounds searchable by 5E8E (Baglin, T. P., et al., J. Thromb. Haemost., 2016, 14: 137-142), and 2QTJ (Bonner, A., et al., J. Immunol., 2008, 180: 1008-1018). Additionally, Hatanaka T. et al., provides great insight into the specificity and high binding affinity of IgA to OPT-1 peptides (J Biol Chem., 2012, 287(51), 43126-43136.).

In alternative embodiments, the IgA Targeting Ligand is a peptide ligand found, for example, in Heineke et al. 2017, Eur. J. Immunol. 47:1835-1845.

Representative IgA Targeting Ligands are provided in FIG. 1.

Additional representative IgA Targeting Ligands include:

SEQ ID NO: 1
MLKKIE (Jerlstrom et al. Infect. Immun. 1996 July; 64(7):2787-2793;
SEQ ID NO: 2
Opt-1-HMVCLAYRGRPVCFAL (Hatanaka et al. J. Biol. Chem. Vol. 287, No.
51, pp. 43126-43136, Dec. 14, 2012)
SEQ ID NO: 3
Opt-2-HMVCLSYRGRPVCFSL (Hatanaka et al. J. Biol. Chem. Vol. 287, No.
51, pp. 43126-43136, Dec. 14, 2012)
SEQ ID NO: 4
Opt-3-HQVCLSYRGRPVCFST (Hatanaka et al. J. Biol. Chem. Vol. 287, No.
51, pp. 43126-43136, Dec. 14, 2012)
SEQ ID NO: 5
QMRCLSYKGRRVCLWL (U.S. Pat. No. 9593147)
SEQ ID NO: 6
KRLCLQYKGSKVCFRL (U.S. Pat. No. 9593147)
SEQ ID NO: 7
RMRCLTYRGRRVCLEL (U.S. Pat. No. 9593147)
SEQ ID NO: 8
SMRCLQYRGSRVCLTL (U.S. Pat. No. 9593147)
SEQ ID NO: 9
HLRCLRYKGTRVCFSL (U.S. Pat. No. 9593147)
SEQ ID NO: 10
HVRCLSYKGREVCVQL (U.S. Pat. No. 9593147)
SEQ ID NO: 11
PRMCLFIYKGRRVCIPY (U.S. Pat. No. 9593147)
SEQ ID NO: 12
HMRCLHYKGRRVCFLL (U.S. Pat. No. 9593147)
SEQ ID NO: 13
HKRCLHYRGRMVCFLI (U.S. Pat. No. 9593147)
SEQ ID NO: 14
QKRCLKYKGSRVCFFL (U.S. Pat. No. 9593147)
SEQ ID NO: 15
HVRCLRYRGKNVCFLL (U.S. Pat. No. 9593147)
SEQ ID NO: 16
SDVCLRYRGRPVCFQV (U.S. Pat. No. 9593147)
SEQ ID NO: 17
RDVCLRYRGRPVCFQV (U.S. Pat. No. 9593147)
SEQ ID NO: 18
HDVCLRYRGRPVCFQV (U.S. Pat. No. 9593147)
SEQ ID NO: 19
SMVCLRYRGRPVCFQV (U.S. Pat. No. 9593147)
SEQ ID NO: 20
SAVCLRYRGRPVCFQV (U.S. Pat. No. 9593147)
SEQ ID NO: 21
SDVCLNYRGRPVCFQV (U.S. Pat. No. 9593147)
SEQ ID NO: 22
SDVCLHYRGRPVCFQV (U.S. Pat. No. 9593147)
SEQ ID NO: 23
SDVCLAYRGRPVCFQV (U.S. Pat. No. 9593147)
SEQ ID NO: 24
SDVCLRYRGRPVCFAV (U.S. Pat. No. 9593147)
SEQ ID NO: 25
SDVCLRYRGRPVCFQL (U.S. Pat. No. 9593147)
SEQ ID NO: 26
SDVCLRYRGRPVCFQA (U.S. Pat. No. 9593147)
SEQ ID NO: 27
HMVCLSYRGRPVCF (U.S. Pub. No. 20150044701)
SEQ ID NO: 28
HMVCLSYRGRPVCFS (U.S. Pub. No. 20150044701)
SEQ ID NO: 29
HQVCLSYRGQPVCFSL (U.S. Pub. No. 20150044701)
SEQ ID NO: 30
HQVCLSYRGRPTCFSL (U.S. Pub. No. 20150044701)
SEQ ID NO: 31
HQVCLSYRGRPVCYSL (U.S. Pub. No. 20150044701)
SEQ ID NO: 32
HQVCLSYRGQPVCFST (U.S. Pub. No. 20150044701)
SEQ ID NO: 33
HQVCLSYRGRPTCFST (U.S. Pub. No. 20150044701)
SEQ ID NO: 34
HQVCLSYRGQPTCFST (U.S. Pub. No. 20150044701)

In certain embodiments the IgA Targeting Ligand is

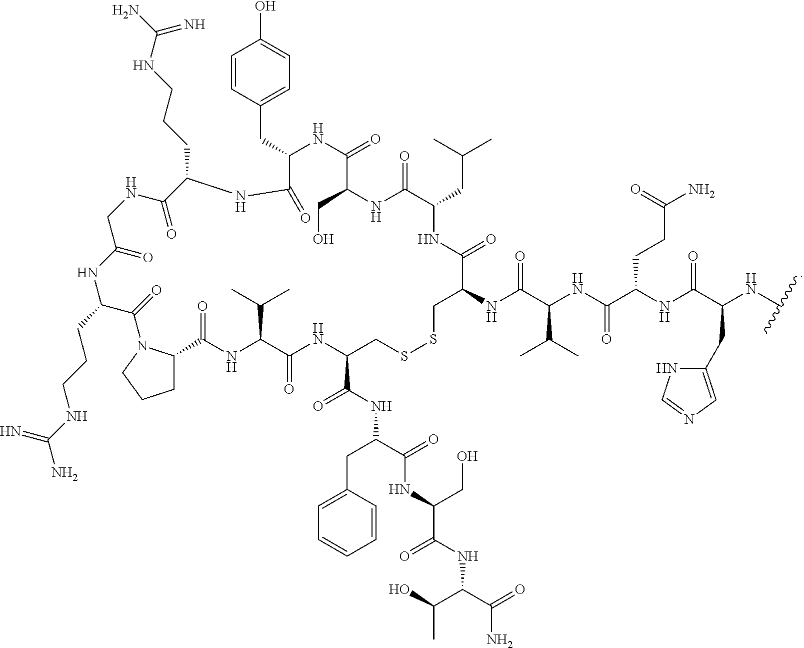

In certain embodiments, the IgA is aberrantly glycosylated IgA, for example galactose deficient IgA. The IgA in the mesangial deposits found in the patients with IgA nephropathy is IgA1 and is aberrantly glycosylated. These IgA1 antibodies have certain O-linked glycans being deficient in galactose. The IgA1 in the circulation of patients with IgAN also carries Gal-deficient O-glycans. In certain embodiments, the Immunoglobulin targeting ligand is an antibody that recognizes galactose deficient IgA. In certain embodiments, the Immunoglobulin Targeting Ligand is an IgA-binding protein described in WO 2015/127438, US2013/0059318, US20120266260, U.S. Pat. Nos. 8,440,191 and 9,655,963; each of which is incorporated by reference herein. In certain embodiments, the Immunoglobulin Targeting Ligand is an IgA-binding lectin described in WO2013172347, incorporated by reference herein.

### Immunoglobulin G (IgG)

Immunoglobulin G (IgG) mediates a range of autoimmune, infectious and metabolic diseases, including systemic fibroinflammatory disease. In addition, overexpression of IgG4 is associated with IgG4-related diseases, which generally include multiple organs, and disorders include type 1 autoimmune pancreatitis, interstitial nephritis, Riedel's thyroiditis, storiform fibrosis, Mikulicz's disease, Küttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementemic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenia gravis, ankylosing spondylitis, primary Sjögren's syndrome, psoriatic arthritis, systemic lupus erythematosus (SLE), sclerosing cholangitis, IgG monoclonal gammopathy, monoclonal gammopathy of undetermined significance (MGUS), melanoma, bullous pemphigoid, Goodpasture disease, encephalitis, thrombotic thrombocytopenic purpura, immune thrombocytopenia, chronic inflammatory polyneuropathy, limbic encephalitis, neuromyotonia, Morvan syndrome, pemphigus foliaceus, pemphigus vulgaris, REM and non-REM parasomnia, and membranous nephropathy, multiple sclerosis, hyperthyroid Grave's disease, epidermolysis bullosa acquisita, pemphigoid gestationis, anti-p200 pemphigoid, and paraneoplastic pemphigus, among others.

Specific degradation of IgG can be accomplished through the use of an IgG-specific Immunoglobulin Targeting Ligand. In certain embodiments, the Immunoglobulin Targeting Ligand binds to the Fc region of IgG. In certain embodiments the IgG-specific Immunoglobulin Targeting Ligand is an Fc-binding peptide. In certain embodiments, the IgG-specific Immunoglobulin Targeting Ligand is Fc-BP2. In certain embodiments, the IgG-specific Immunoglobulin Targeting Ligand is Fc-III.

In certain alternative embodiments any compound drawn herein with stereochemistry drawn in the Targeting Ligand is also described herein without stereochemistry. For example, in certain embodiments:

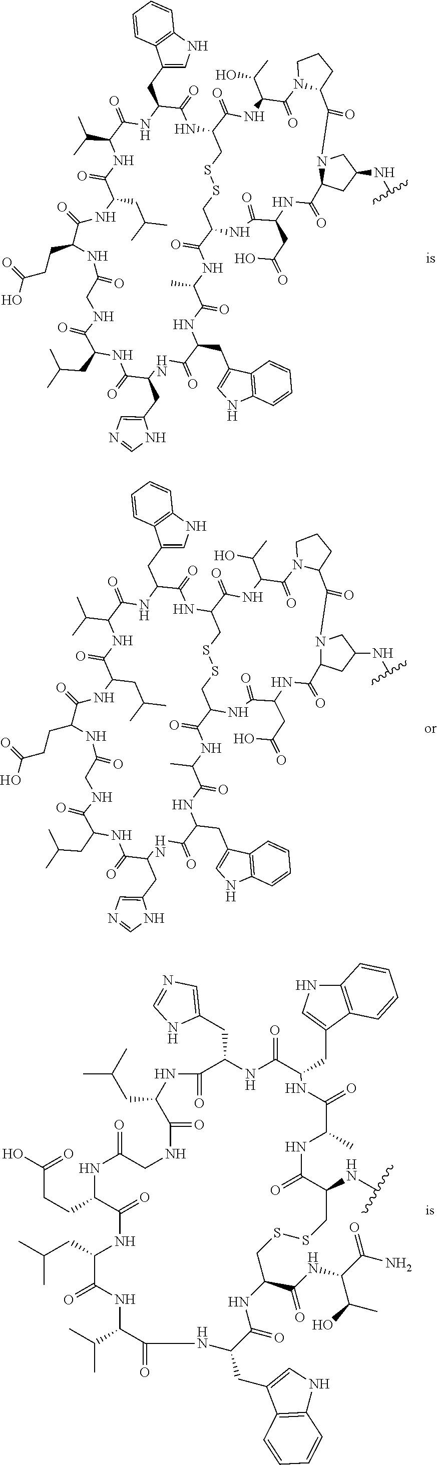

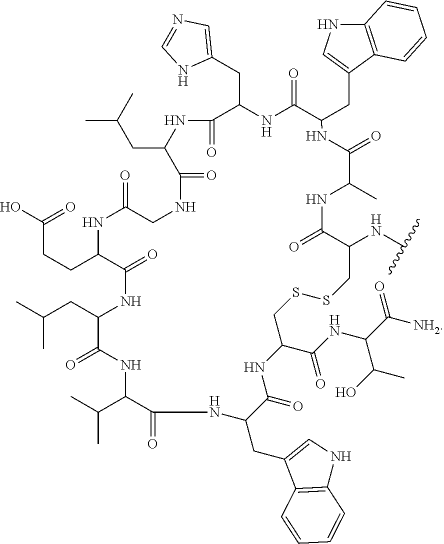 [SMILES: *NC1CSSCC(C(=O)NC(C(N)=O)C(C)O)NC(=O)C(Cc2c[nH]c3ccccc23)NC(=O)C(C(C)C)NC(=O)C(CC(C)C)NC(=O)C(CCC(=O)O)NC(=O)CNC(=O)C(CC(C)C)NC(=O)C(Cc2cnc[nH]2)NC(=O)C(Cc2c[nH]c3ccccc23)NC(=O)C(C)NC1=O]

The Protein Data Bank website provides the crystal structure of IgG searchable by 1H3X (Krapp, S., et al., J. Mol. Biol., 2003, 325: 979); and 5V43 (Lee, C. H., et al., Nat. Immunol., 2017, 18: 889-898); as well as the crystal structure of IgG bound to various compounds searchable by 5YC5 (Kiyoshi M., et al., Sci. Rep., 2018, 8: 3955-3955); 5XJE (Sakae Y., et al., Sci. Rep., 2017, 7: 13780-13780); 5GSQ (Chen, C. L., et al., ACS Chem. Biol., 2017, 12: 1335-1345); and 1HZH (Saphire E. O., et al., Science, 2001, 293: 1155-1159). Additionally, Kiyoshi, M., et al., provides insight into the structural basis for binding of human IgG1 to its high-affinity human receptor FcγRI. (Kiyosi M., et al., Nat Commun., 2015, 6, 6866).

Representative IgG Targeting Ligands are provided in FIG. 1.

Additional representative IgG Targeting Ligands include:

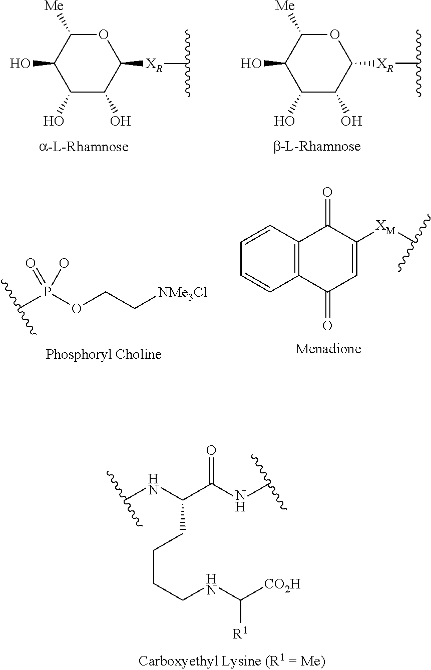

wherein X_{R} is O, S, NH, or N—C₁-C₃ alkyl; and
X_{M} is O, S, NH, or N—C₁-C₃ alkyl.

In other embodiments the IgG Targeting Ligand is selected from:

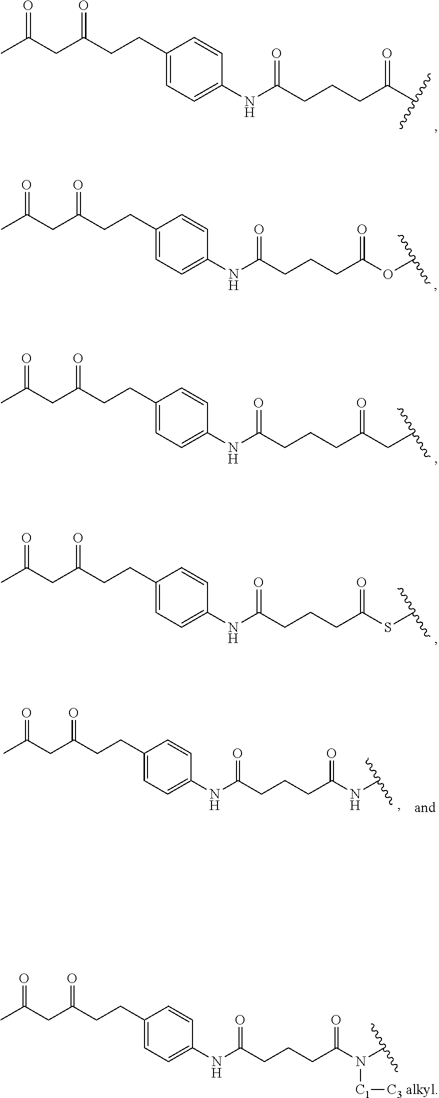

In some embodiments, the IgG Targeting Ligand is a group according to the chemical structure:

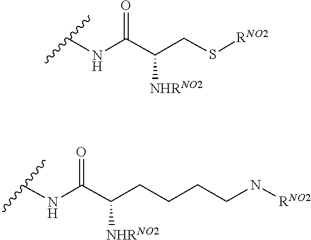

wherein R^{N02} is a dinitrophenyl group optionally linked through CH₂, S(O), S(O)₂, —S(O)₂O, —OS(O)₂, or OS(O)₂O.

In certain embodiments the IgG Targeting Ligand is selected from:

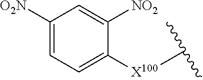

wherein X¹⁰⁰ is selected from 0, CH₂, NH, N—C₁-C₃ alkyl, NC(O)C₁-C₃ alkyl, S(O), S(O)₂, —S(O)₂O, —OS(O)₂, or OS(O)₂O.

In some embodiments, the IgG Targeting Ligand is a 3-indoleacetic acid group according to the chemical structure:

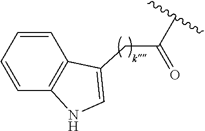

where k″″ is 1-4 (preferably 2-3, most often 3) or a

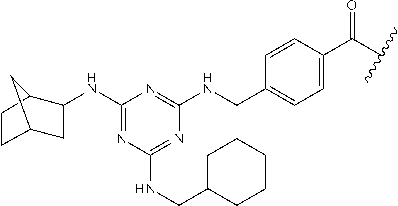 [SMILES: *C(=O)c1ccc(CNc2nc(NCC3CCCCC3)nc(NC3CC4CCC3C4)n2)cc1]

group.

In some embodiments, the IgG Targeting Ligand is a peptide. Nonlimiting examples of IgG Targeting Ligand peptides include:
SEQ ID NO:35 PAM (RTY)₄K2KG (Fassina, et al, J. Mol. Recognit. 1996, 9, 564-569)

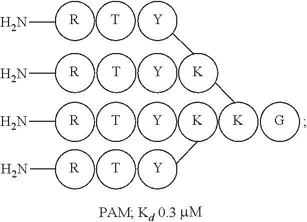

D-PAM, wherein the amino acids of the PAM sequence are all D-amino acids (Verdoliva, et al, J. Immunol. Methods, 2002, 271, 77-88) SEQ ID NO:36 (RTY)₄K2KG D-PAM-Φ, wherein the amino acids of the PAM sequence are all D-amino acids with further modifications wherein the four N-terminal arginines are acetylated with phenylacetic acid (Dinon, et al J. Mol. Recognit. 2011, 24, 1087-1094) SEQ ID NO:37 (RTY)₄K2KG SEQ ID NO:38 TWKTSRISIF (Krook, et al, J. Immunol. Methods 1998, 221, 151-157) SEQ ID NO:39 FGRLVSSIRY (Krook, et al, J. Immunol. Methods 1998, 221, 151-157) SEQ ID NO:40 Fc-III (DCAWHLGELVWCT-NH2) (DeLano et al, Science 2000, 287, 1279-1283)

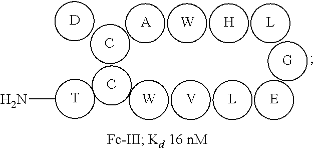

SEQ ID NO: 41
FCBP-Ser DSAWHLGELWST (see WO2014010813)
SEQ ID NO: 42
DCHKRSFWADNCT (see WO2014010813)
SEQ ID NO: 43
DCRTQFRPNQTCT (see WO2014010813)
SEQ ID NO: 44
DCQLCDFWRTRCT (see WO2014010813)
SEQ ID NO: 45
DCFEDFNEQRTCT (see WO2014010813)
SEQ ID NO: 46
DCLAKFLKGKDCT (see WO2014010813)
SEQ ID NO: 47
DCWHRRTHKTFCT (see WO2014010813)
SEQ ID NO: 48
DCRTIQTRSCT (see WO2014010813)
SEQ ID NO: 49
DCIKLAQLHSVCT (see WO2014010813)
SEQ ID NO: 50
DCWRHRNATEWCT (see WO2014010813)
SEQ ID NO: 51
DCQNWIKDVHKCT (see WO2014010813)
SEQ ID NO: 52
DCAWHLGELVWCT (see WO2014010813)
SEQ ID NO: 53
DCAFHLGELVWCT (see WO2014010813)
SEQ ID NO: 54
DCAYHLGELVWCT (see WO2014010813)
SEQ ID NO: 55
FcBP-1 PAWHLGELVWP (Kang, et al, J. Chromatogr.
A 2016, 1466, 105-1 12)

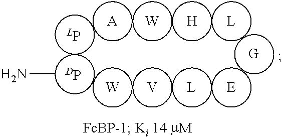

SEQ ID NO: 56
FcBP-2 PDCAWHLGELVWCTP (Dias, et al, J. Am. Chem.
Soc. 2006, 128, 2726-2732)

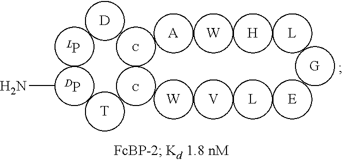

SEQ ID NO: 57
Fc-111-4c CDCAWHLGELVWCTC (Gong, et al, Bioconjug.
Chem. 2016, 27, 1569-1573)

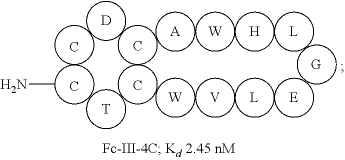

SEQ ID NO: 58
EPIHRSTLTALL (Ehrlich, et al, J. Biochem. Biophys.
Method 2001, 49, 443- 454)
SEQ ID NO: 59
APAR (Camperi, et al, Biotechnol. Lett. 2003, 25,
1545-1548)
SEQ ID NO: 60
FcRM (CFHH)₂KG (Fc Receptor Mimetic, Verdoliva,
et al., ChemBioChem 2005, 6, 1242-1253)

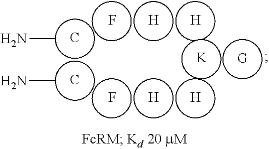

SEQ ID NO: 61
HWRGWV (Yang, et al., J Peptide Res. 2006, 66, 1 1 0-137)
SEQ ID NO: 62
HYFKFD (Yang, et al, J. Chromatogr. A 2009, 1216, 910-918)
SEQ ID NO: 63
HFRRHL (Menegatti, et al, J. Chromatogr. A 2016, 1445, 93-104)
SEQ ID NO: 64
HWCitGWV (Menegatti, et al, J. Chromatogr. A 2016, 1445, 93-104)
SEQ ID NO: 65
HWmetCitGWmetV (U.S. Pat. No. 10,266,566)
SEQ ID NO: 66
D2AAG (Small Synthetic peptide ligand, Lund, et al, J. Chromatogr. A
2012, 1225, 158-167)
SEQ ID NO: 67
DAAG (Small Synthetic peptide ligand, Lund, et al, J. Chromatogr. A
2012, 1225, 158-167);
SEQ ID NO: 68
cyclo[(Nα-Ac)S(A)-RWHYFK-Lact-E] (Menegatti, et al, Anal. Chem.
2013, 85, 9229-9237);
SEQ ID NO: 69
cyclo[(Nα-Ac)-Dap(A)-RWHYFK-Lact-E] (Menegatti, et al, Anal. Chem.
2013, 85, 9229-9237);
SEQ ID NO: 70
cyclo[Link M-WFRHYK] (Menegatti, et al, Biotechnol. Bioeng. 2013,
110, 857-870);
SEQ ID NO: 71
NKFRGKYK (Sugita, et al, Biochem. Eng. J. 2013, 79, 33-40);
SEQ ID NO: 72
NARKFYKG (Sugita, et al, Biochem. Eng. J. 2013, 79, 33-40);
SEQ ID NO: 73
FYWHCLDE (Zhao, et al, Biochem. Eng. J. 2014, 88, 1-11);
SEQ ID NO: 74
FYCHWALE (Zhao, et al, J Chromatogr. A 2014, 1355, 107-114);
SEQ ID NO: 75
FYCHTIDE (Zhao, et al., Z Chromatogr. A 2014, 1359, 100-111);
SEQ ID NO: 76
Dual 1/3 (FYWHCLDE-FYCHTIDE) (Zhao, et al, J. Chromatogr. A 2014,
1369, 64-72);
SEQ ID NO: 77
RRGW (Tsai, et al, Anal. Chem. 2014, 86, 293 1-2938);
SEQ ID NO: 78
KHRFNKD (Yoo and Choi, BioChip J. 2015, 10, 88-94);
SEQ ID NO: 79
CPSTHWK (Sun et al. Polymers 2018, 10, 778);
SEQ ID NO: 80
NVQYFAV (Sun et al. Polymers 2018, 10, 778);
SEQ ID NO: 81
ASHTQKS (Sun et al. Polymers 2018, 10, 778);
SEQ ID NO: 82
QPQMSHM (Sun et al. Polymers 2018, 10, 778);
SEQ ID NO: 83
TNIESLK (Sun et al. Polymers 2018, 10, 778);
SEQ ID NO: 84
NCHKCWN (Sun et al. Polymers 2018, 10, 778);
SEQ ID NO: 85
SHLSKNF (Sun et al. Polymers 2018, 10, 778).

In some embodiments the IgG Targeting Ligand is specific for IgG4.

In some embodiments the IgG4 specific Targeting Ligand is described in Gunnarsson et al. Biomolecular Engineering 2006, 23, 111-117.

In some embodiments the IgG4 specific targeting ligand is selected from

SEQ ID NO: 86
FDLLEHFY
and
SEQ ID NO: 87
DLLHHFDYF.

Additional IgG Targeting Ligands include

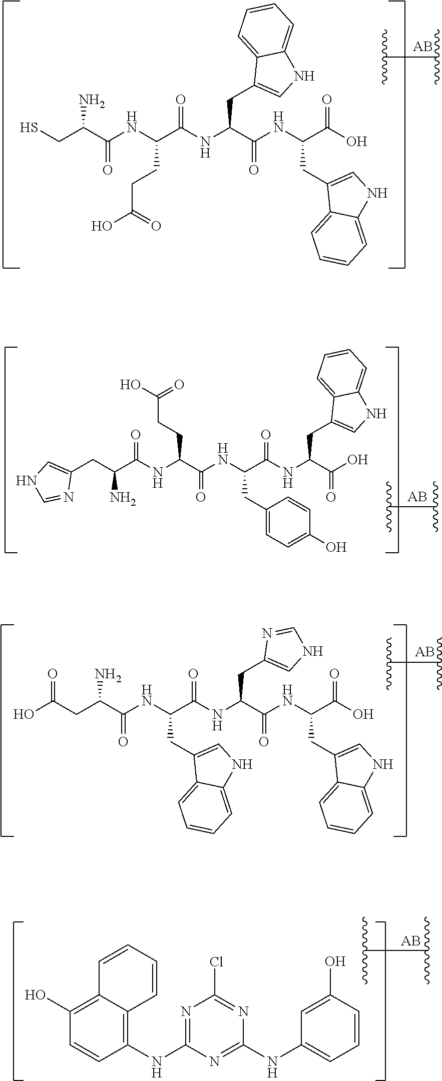

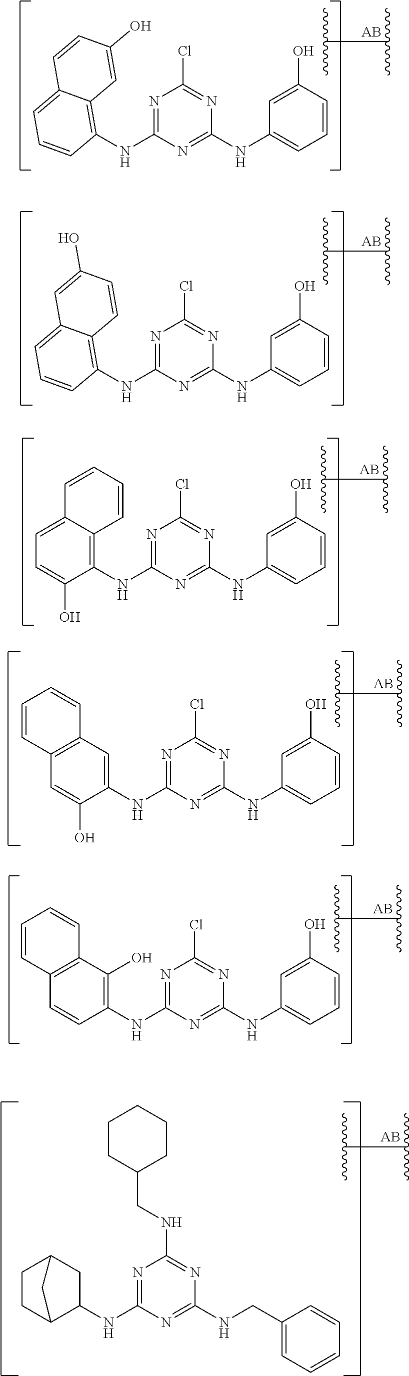

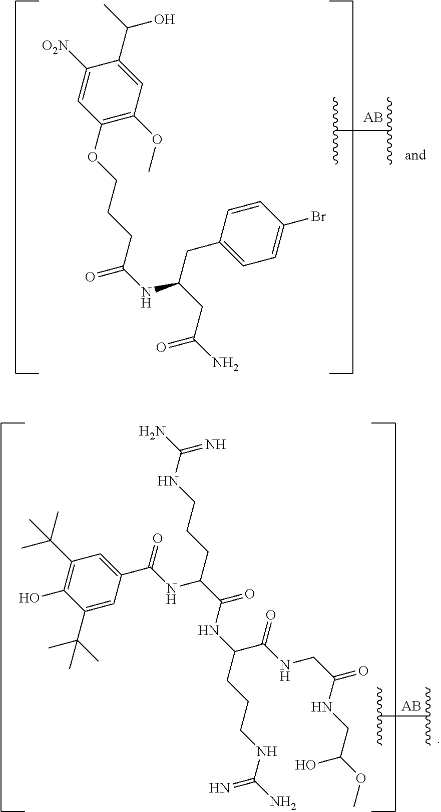

In alternative embodiments a hydroxyl, amine, amide, or carboxylic acid group in an Extracellular Protein Targeting Ligand drawn herein is capped with a protecting group. For example in this embodiment:

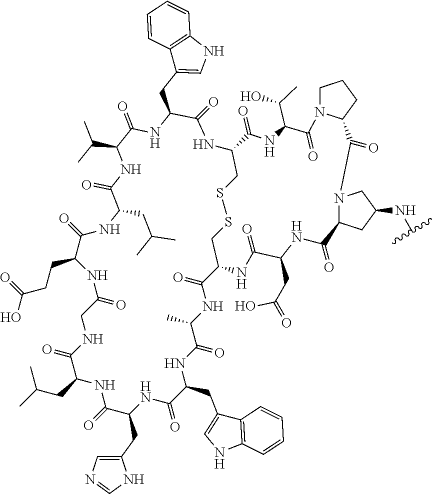 [SMILES: *N[C@H]1C[C@H]2C(=O)N[C@@H](CC(=O)O)C(=O)N[C@H]3CSSC[C@H](NC(=O)[C@H](Cc4c[nH]c5ccccc45)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc4cnc[nH]4)NC(=O)[C@H](Cc4c[nH]c5ccccc45)NC(=O)[C@H](C)NC3=O)C(=O)N[C@@H]([C@@H](C)O)C(=O)N3CCC[C@@H]3C(=O)N2C1]

can be

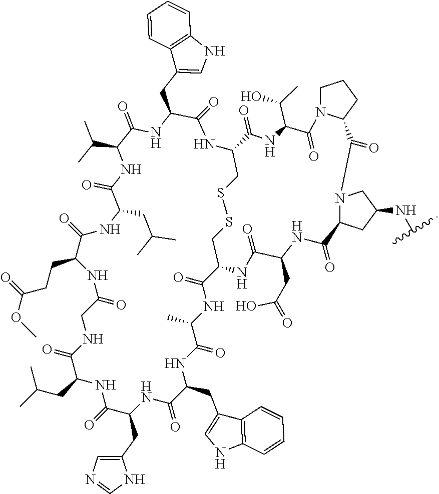 [SMILES: *N[C@H]1C[C@H]2C(=O)N[C@@H](CC(=O)O)C(=O)N[C@H]3CSSC[C@H](NC(=O)[C@H](Cc4c[nH]c5ccccc45)NC(=O)[C@H](C(C)C)NC(=O)[C@H](CC(C)C)NC(=O)[C@H](CCC(=O)OC)NC(=O)CNC(=O)[C@H](CC(C)C)NC(=O)[C@H](Cc4cnc[nH]4)NC(=O)[C@H](Cc4c[nH]c5ccccc45)NC(=O)[C@H](C)NC3=O)C(=O)N[C@@H]([C@@H](C)O)C(=O)N3CCC[C@@H]3C(=O)N2C1]

In alternative embodiments a hydroxyl, amine, amide, or carboxylic acid group in an Extracellular Protein Targeting Ligand drawn herein is used as the attachment point to Linker instead of the drawn attachment point. For example in this embodiment:

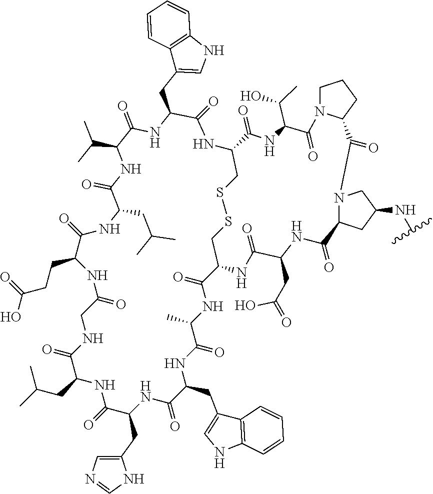 [SMILES: *N[C@H]1C[C@H]2C(=O)N[C@@H](CC(=O)O)C(=O)N[C@H]3CSSC[C@H](NC(=O)[C@H](Cc4c[nH]c5ccccc45)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc4cnc[nH]4)NC(=O)[C@H](Cc4c[nH]c5ccccc45)NC(=O)[C@H](C)NC3=O)C(=O)N[C@@H]([C@@H](C)O)C(=O)N3CCC[C@@H]3C(=O)N2C1]

can be

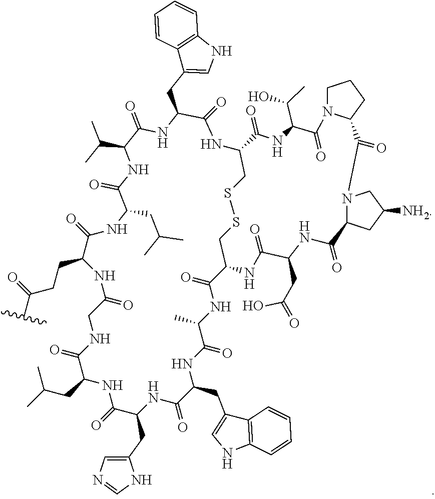 [SMILES: *C(=O)CC[C@@H]1NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC1=O)C(=O)N[C@@H]([C@@H](C)O)C(=O)N1CCC[C@@H]1C(=O)N1C[C@@H](N)C[C@H]1C(=O)N[C@@H](CC(=O)O)C(=O)N2]

### Immunoglobulin E (IgE)

Immunoglobulin E (IgE) is a strong mediator of allergic disease, including but not limited to, atopic asthma, allergic rhinitis, atopic dermatitis, cutaneous contact hypersensitivity, IgE-mediated food allergy, IgE-mediated animal allergies, allergic conjunctivitis, allergic urticaria, anaphylactic shock, nasal polyposis, keratoconjunctivitis, mastocytosis, eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, monoclonal gammopathy of undetermined significance (MGUS), pemphigus vulgaris, mucus membrane pemphigoid, chronic urticaria, autoimmune uveitis, rheumatoid arthritis, autoimmune pancreatitis, and allergic rhinoconjunctivitis among others.

In certain embodiments the Immunoglobulin Targeting Ligand is:

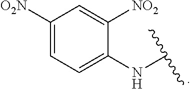 [SMILES: *Nc1ccc([N+](=O)[O-])cc1[N+](=O)[O-]]

In certain embodiments the IgE Targeting Ligand is selected from

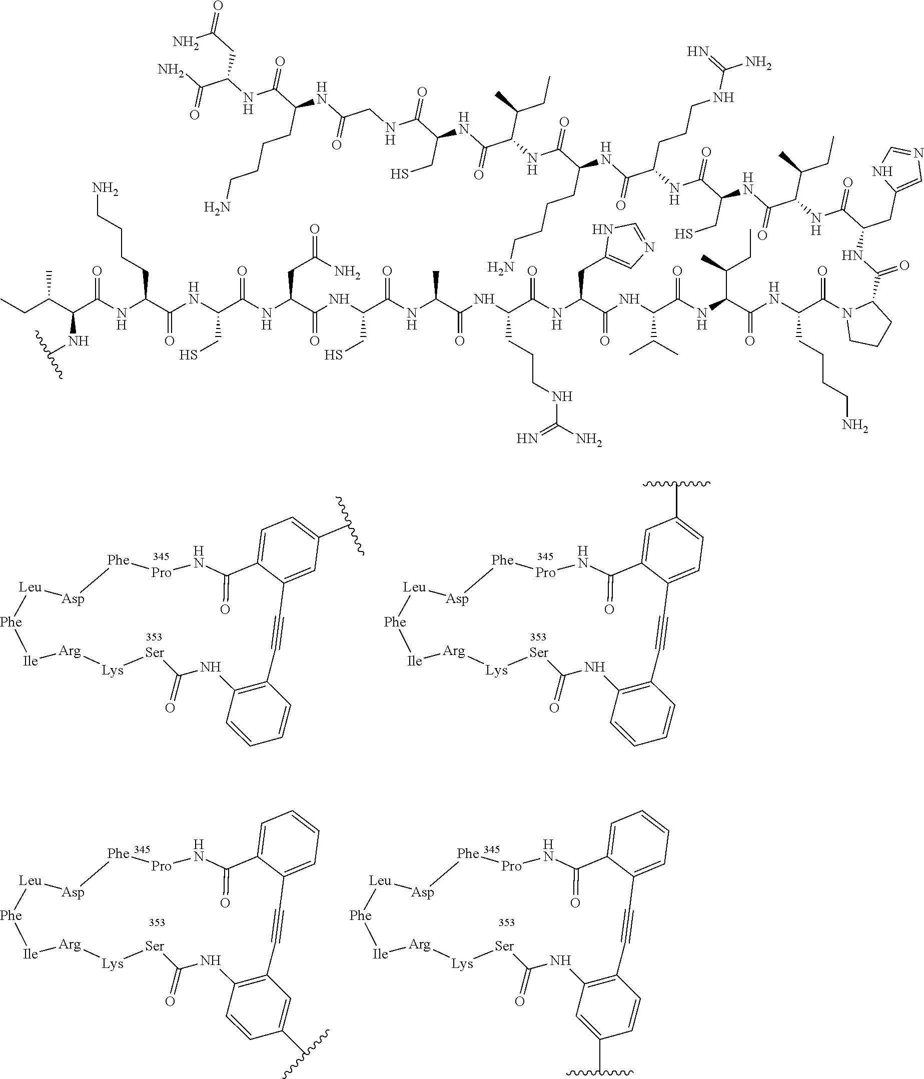

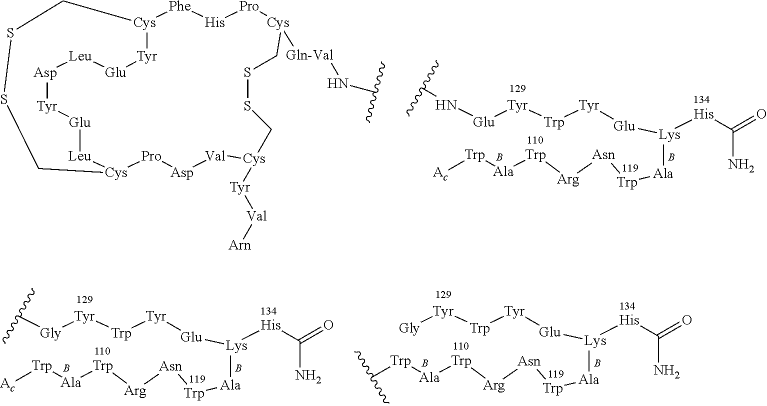

### IgM Autoantibodies

In some embodiments the Target Extracellular Protein is IgM or an anti-MAG IgM autoantibody. Myelin-associated glycoprotein (MAG) is a transmembrane glycoprotein that plays a role in glial-axonal interactions in the nervous system. In some patients, IgM anti-MAG antibodies develop leading to neuropathy. Antibody levels can be as high as four-fold over normal, leading to potential nephropathy. Lowering levels of anti-MAG antibodies is associated with clinical response in polyneuropathy.

Representative targeting ligands that bind to Anti-MAG IgM autoantibodies include

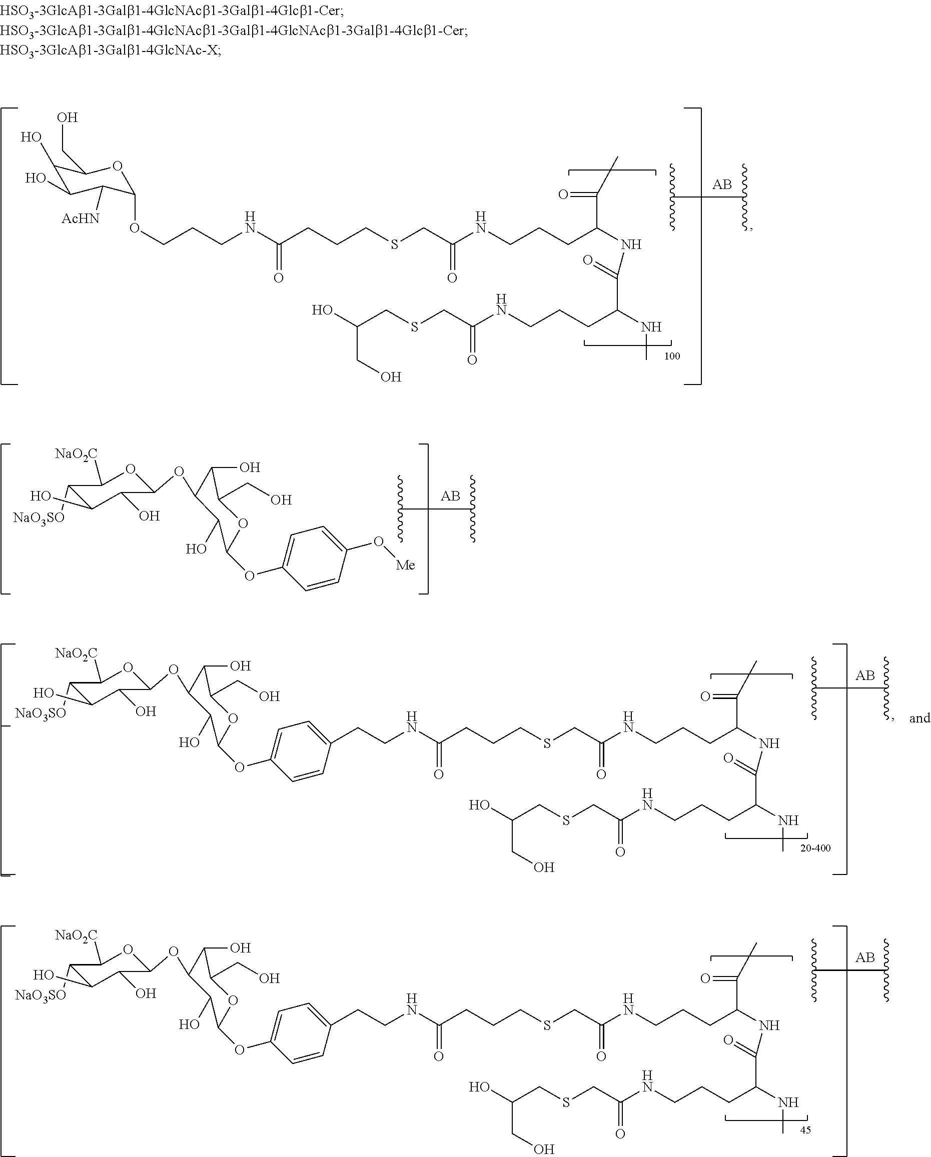

Additional IgM autoantibodies that can be used in the present invention are described in Herrendorff, R. et al. 2017 PNAS Early Edition, doi/10.1073/pnas.1619386114, WO 2018/167230, U.S. Pat. Nos. 9,056,081; 9,994,605; Volshol et al. J. Biol. Chem 1996; Wang et al. 2020; WO 2000/050447; WO 2015/136027; Bunyatov et al. “Synthetic HNK-1 containing glycans provide insight into binding properties of serum antibodies from MAG-neuropathy patients” BioRxiv, 2022; Aliu et al. “Selective inhibition of anti-MAG IgM autoantibody binding to myelin by an antigen-specific glycopolymer” Journal of Neurochemistry 2020; WO 2022/081895; and Simon-Haldi, M. et al. “Identification of a peptide mimic of the L2/HNK-1 carbohydrate epitope” Journal of Neurochemistry 2002, 83, 1380-1388.

In certain embodiments the IgM Targeting Ligand is selected from:

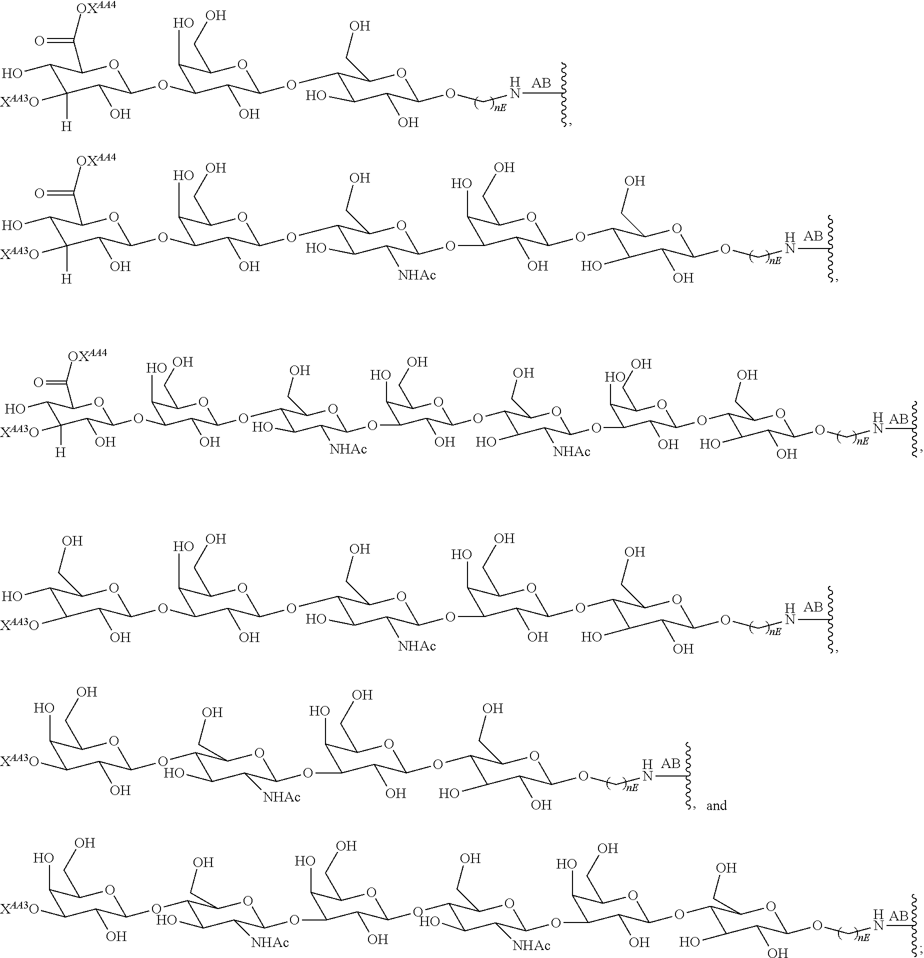

wherein
nE is 1 to 10;
X^{AA3} is selected from H, SO₃, SO₃H, and SO₃Na; and
X^{AA4} is selected from H or Na.

In certain embodiments the IgM Targeting Ligand is selected from:

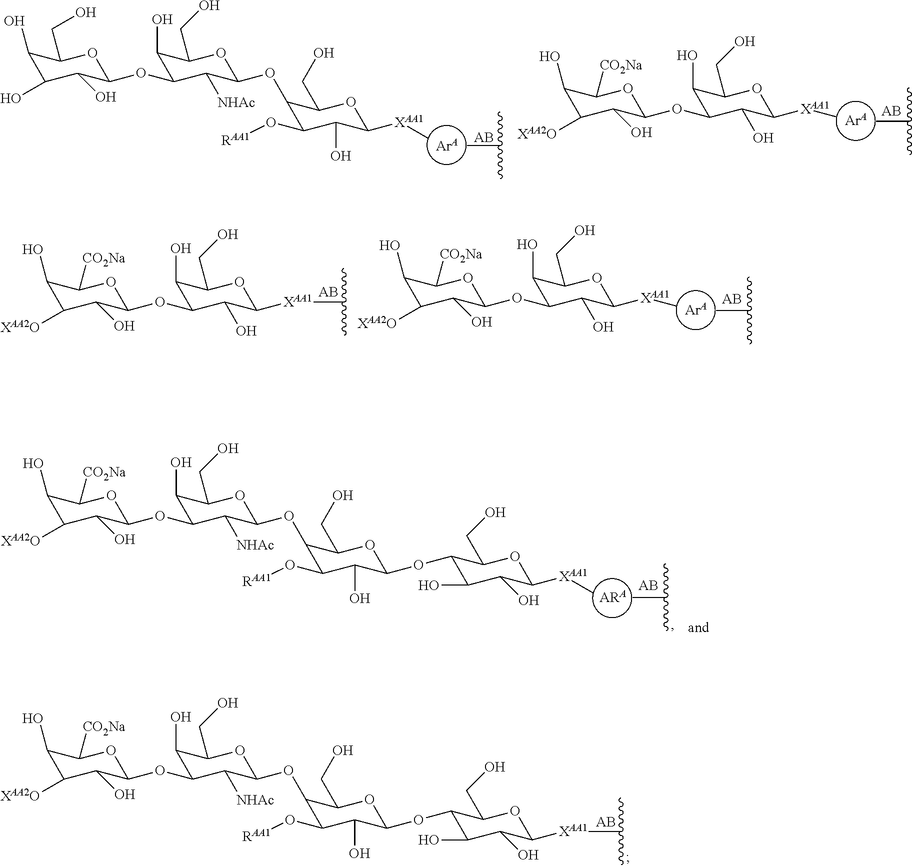

wherein
R^{AA1} is selected from a sialic acid group and an optionally substituted carboxymethyl group;
X^{AA1} is selected from O, S, NR^{AA2}, C(R^{AA2})₂;
X^{AA2} is selected from H and SO₃Na;
R^{AA2} is independently selected from H, C₁-C₄ alkyl, C₁-C₄ alkoxy, benzyl, CH₂CH₂C₆H₅, OCH₂C₆H₅, and OCH₂CH₂C₆H₅;
Ar^{A} is selected from an optionally substituted aryl and an optionally substituted heteroaryl.

In certain embodiments the Targeting Ligand that binds to IgM is a compound of Formula

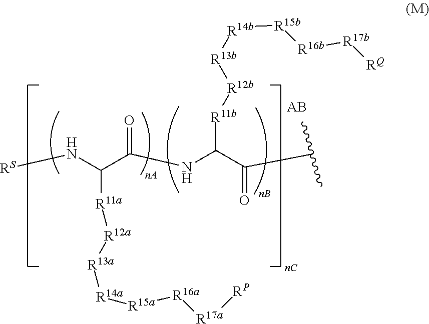

wherein
R^{11a}, R^{11b}, R^{12a}, R^{12b}, R^{13a}, R^{13b}, R^{14a}, R^{14b}, R^{15a}, R^{15b}, R^{16a}, R^{16b}, R^{17a}, and R^{17b} are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, 3, or 4 substituents independently selected from R²¹;
R^{Q} is

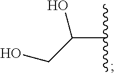 [SMILES: *C(O)CO]

W¹ is selected from H, SO₃⁻, SO₃H, and SO₃Na; and
W² is selected from H or Na;

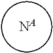

is selected from aryl, heteroaryl, and cycloalkyl;
R^{AA1} is selected from a sialic acid group and an optionally substituted carboxymethyl group;
X^{AA1} is selected from O, S, NR^{AA2}, C(R^{AA2})₂;
X^{AA2} is selected from H and SO₃Na;
R^{AA2} is independently selected from H, C₁-C₄ alkyl, C₁-C₄ alkoxy, benzyl, —CH₂-benzyl, —O-benzyl, and —O—CH₂-benzyl;
R^{S} is hydrogen, alkyl, cycloalkyl, aryl, or heteroaryl.
nA is 10-90% of the polymer and nB is 100%−nA;
nC is 1-500;

In certain embodiments nC is 150-300, 70-150, 40-60, 30-70, 15-30, or 4-5. In certain embodiments, nC is at least about 50, 60, 70, 80, 90, 100, 110, 120, 130, 140, or 150. In certain embodiments, nC is about 75, 80, 85, 90, 95, or 100.

R^{P} is selected from compounds of formula:

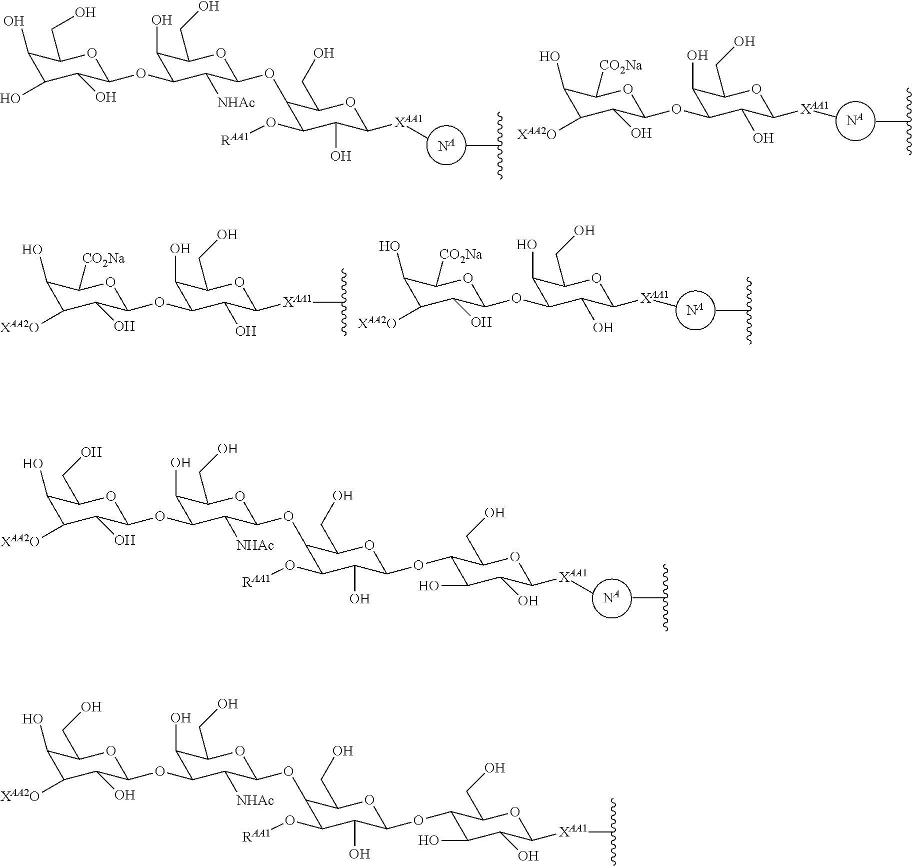

In certain embodiments the IgM Targeting Ligand is selected from

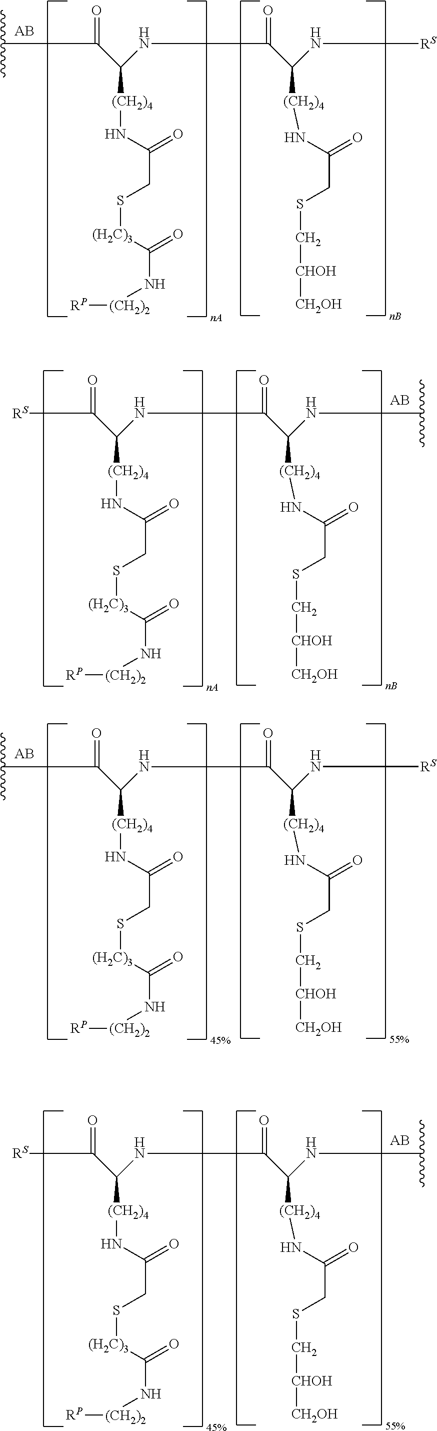

In certain embodiments the IgM Targeting Ligand is selected from

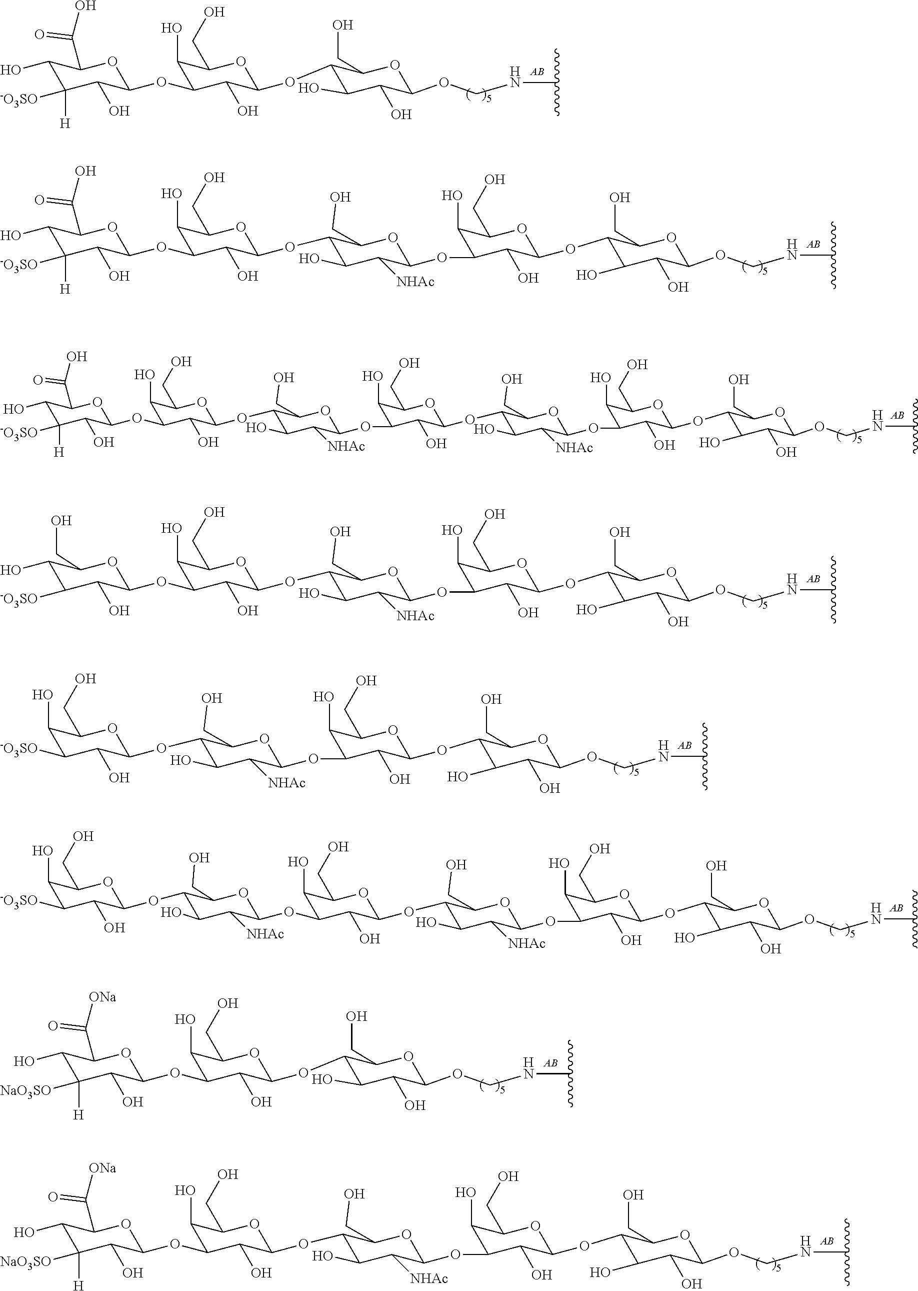

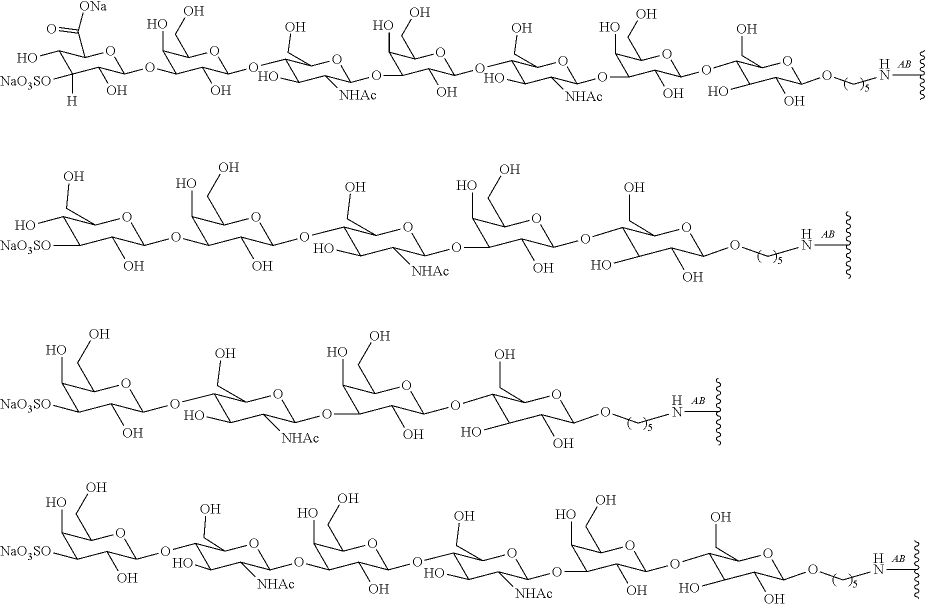

In certain embodiments the IgM Targeting Ligand is selected from

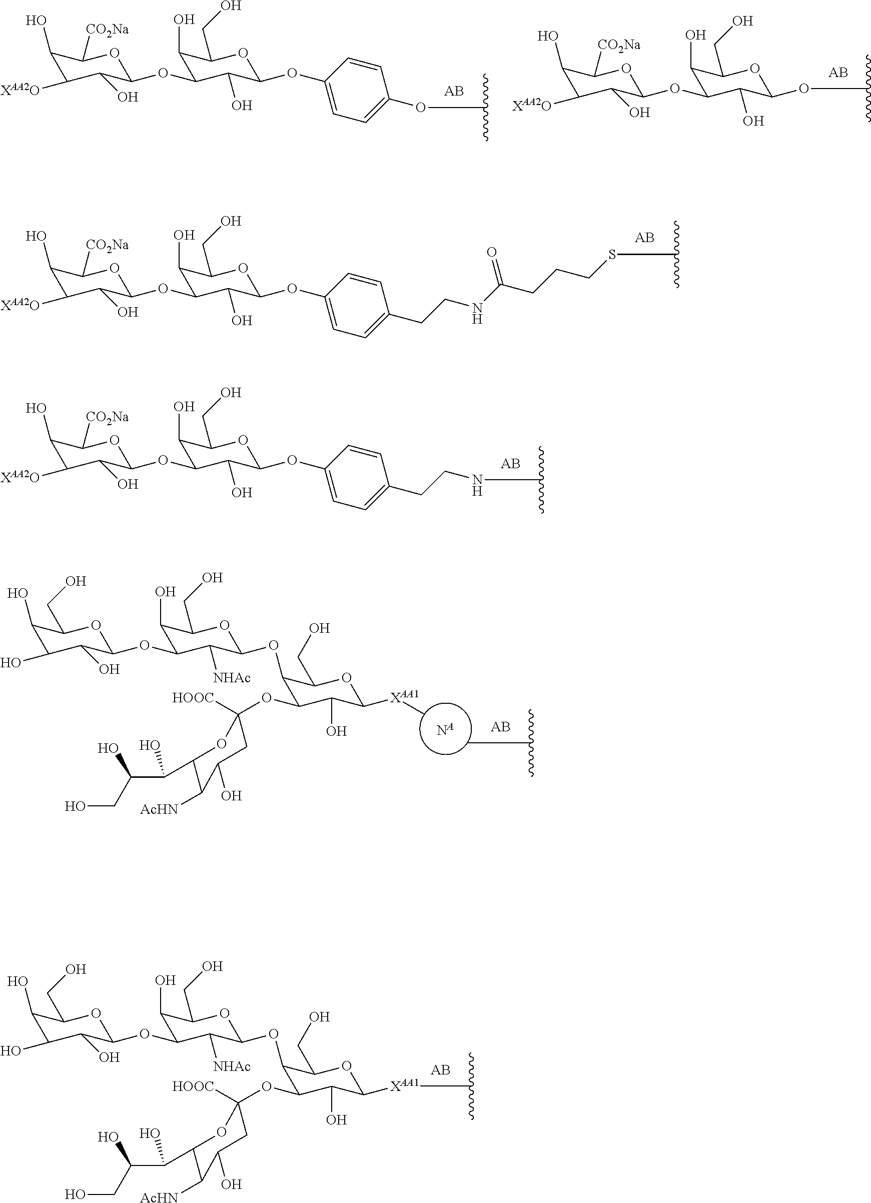

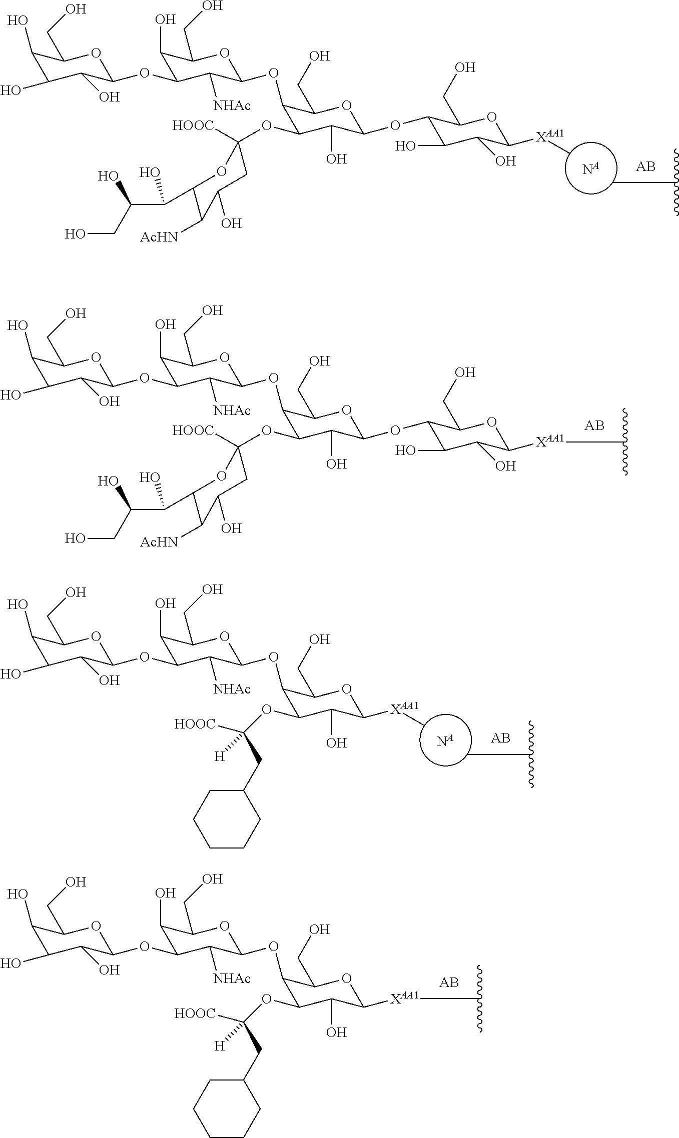

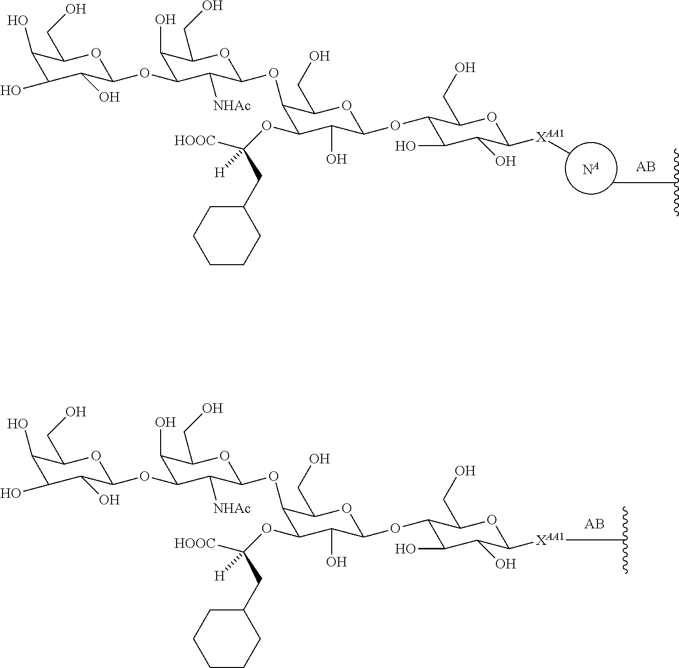

Non-limiting examples of IgM degrading compounds include:

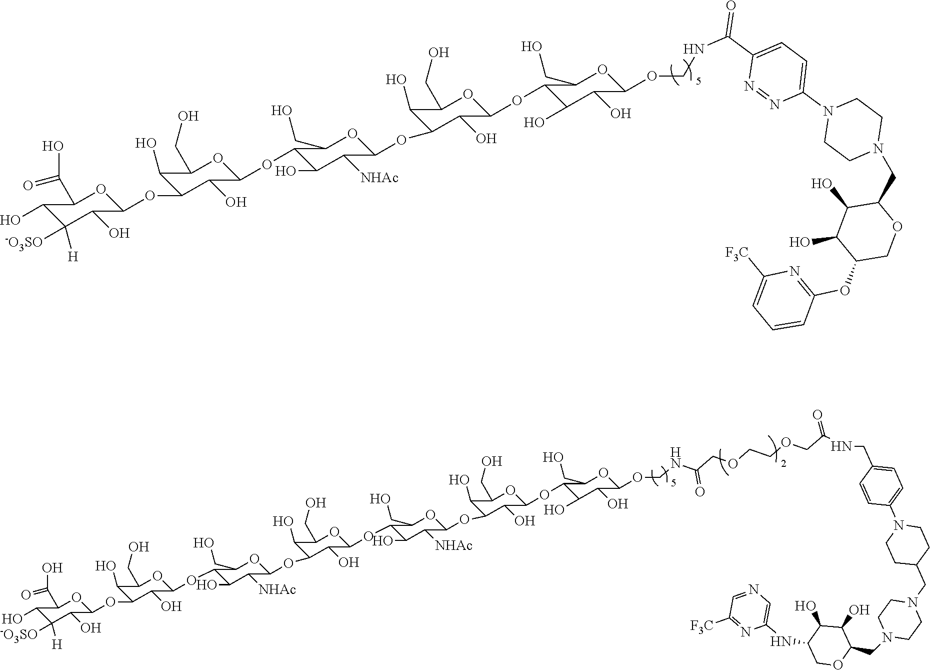

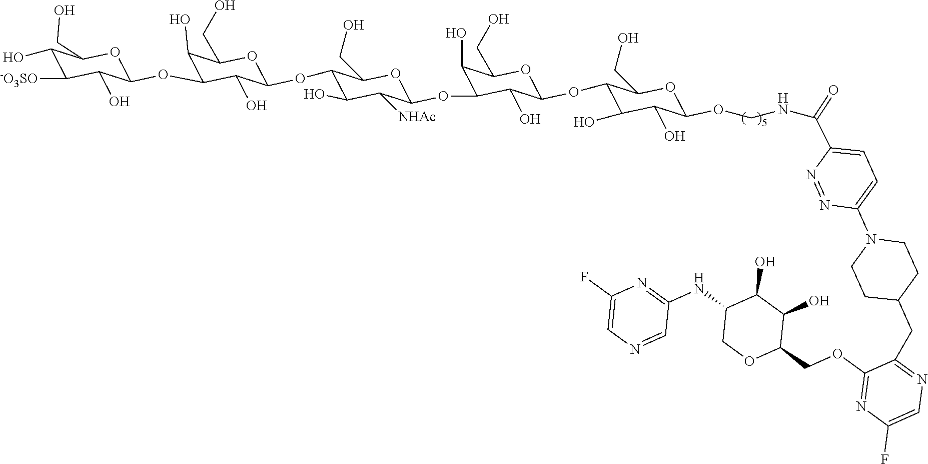

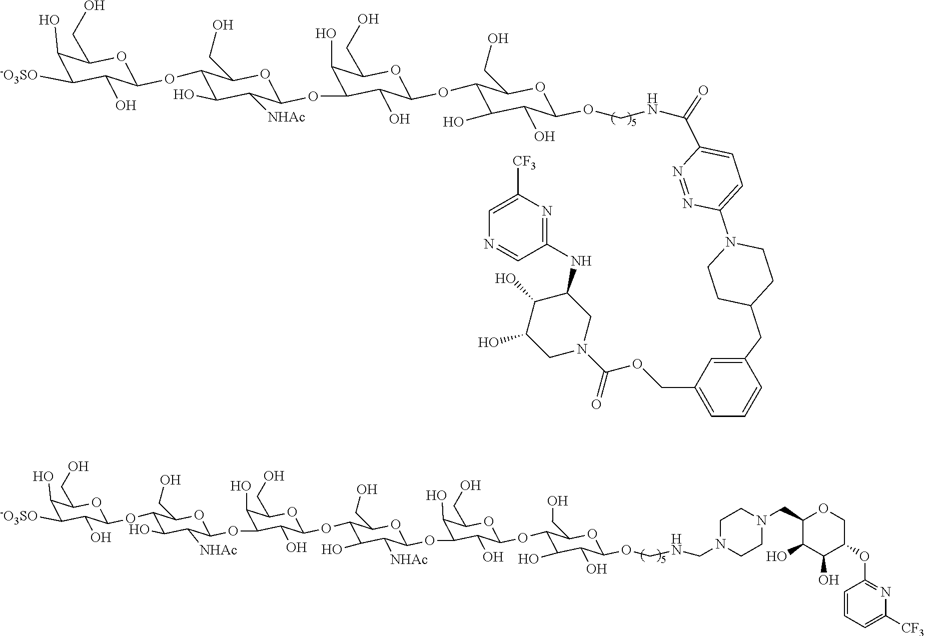

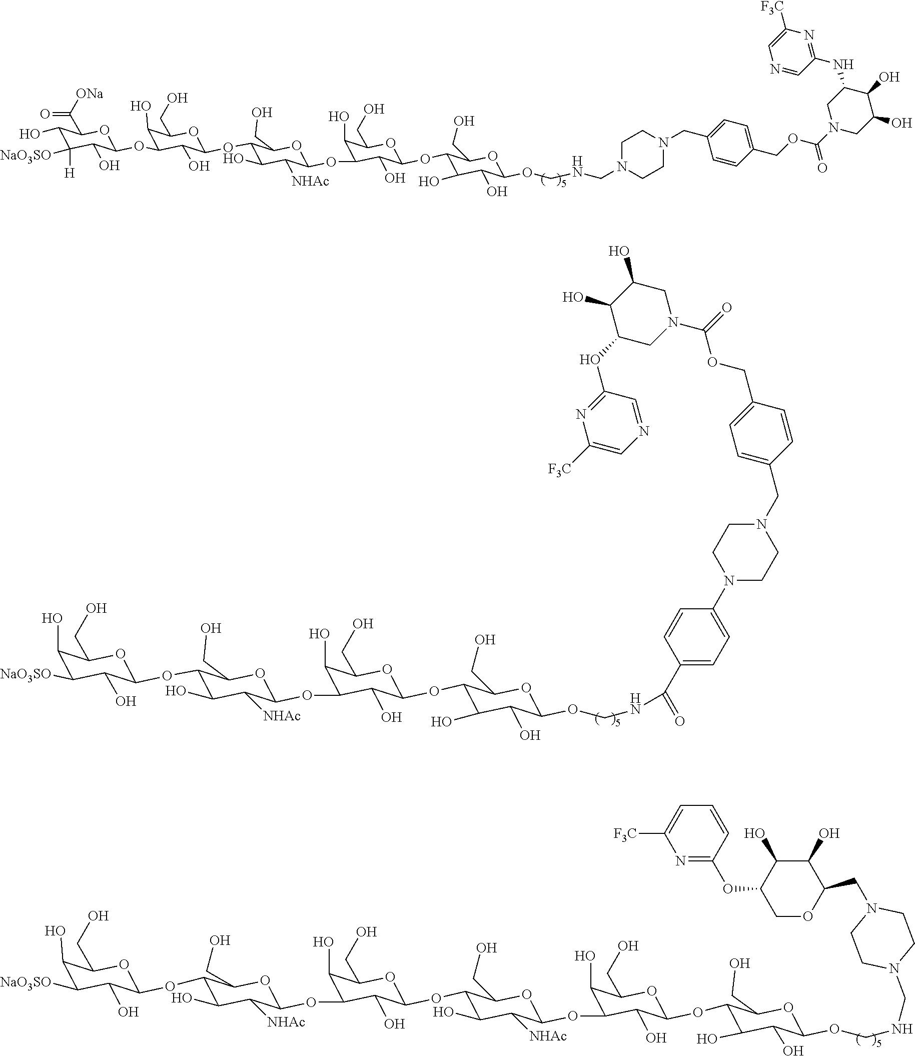

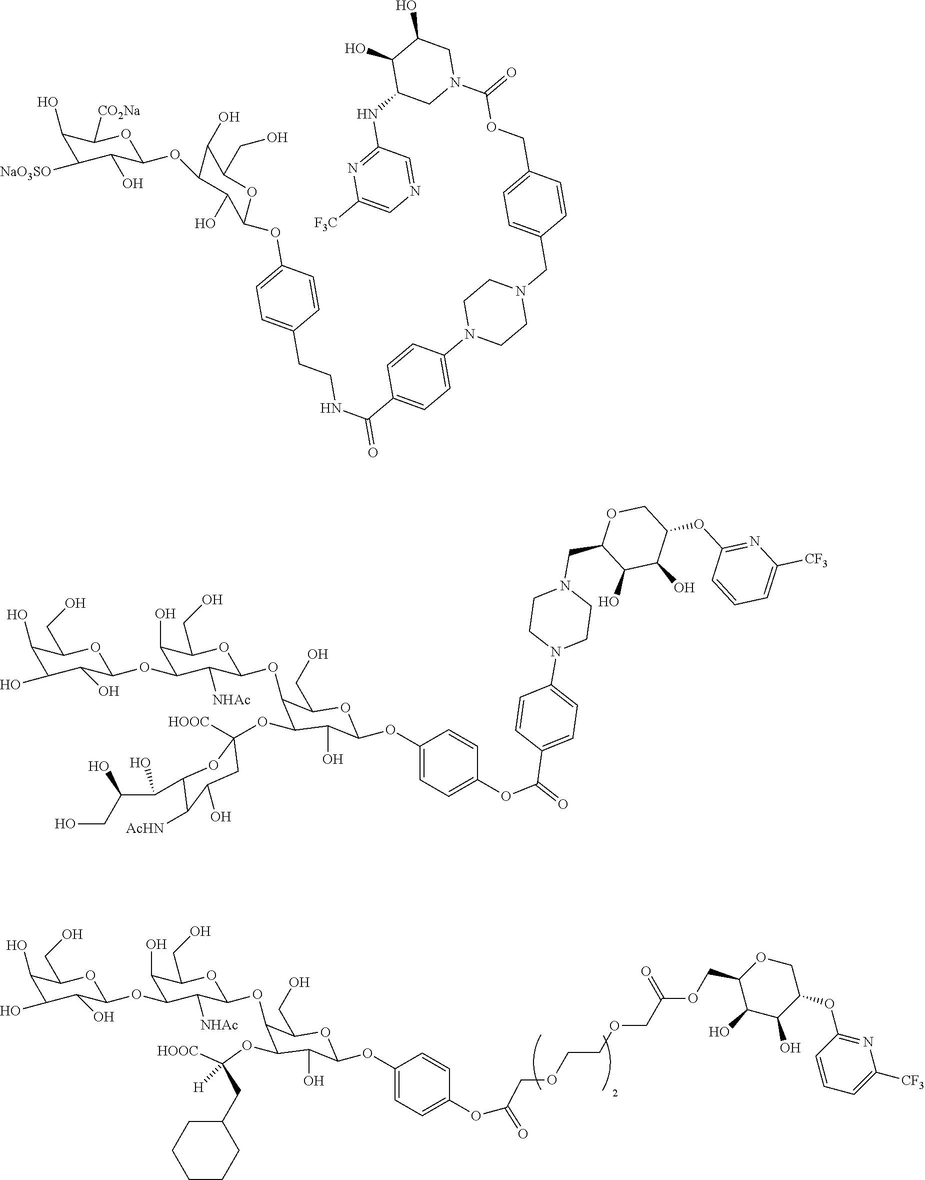

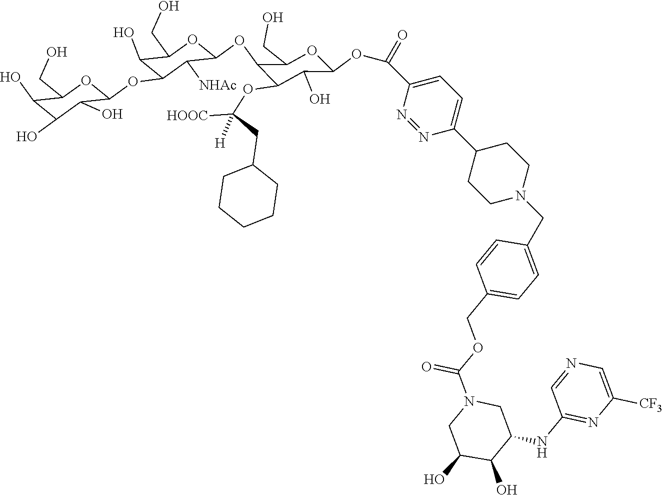 [SMILES: CC(=O)NC1C(OC2C(CO)OC(OC(=O)c3ccc(C4CCN(Cc5ccc(COC(=O)N6C[C@H](Nc7cncc(C(F)(F)F)n7)[C@@H](O)[C@@H](O)C6)cc5)CC4)nn3)C(O)C2O[C@@H](CC2CCCCC2)C(=O)O)OC(CO)C(O)C1OC1OC(CO)C(O)C(O)C1O]

or a pharmaceutically acceptable salt thereof.

Wherein in all the above structures sodium and negative charges can be optionally replaced with hydrogen.

In certain aspects an IgM degrading compound is provided of Formula:

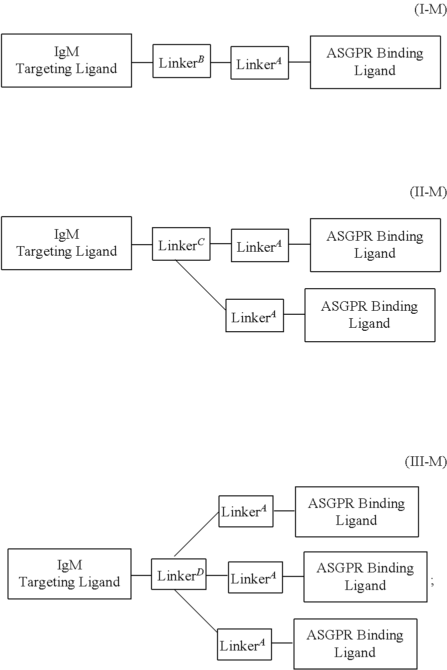

or a pharmaceutically acceptable salt thereof.

In certain embodiments the ASGPR Binding Ligand used in Formula I-M, II-M, or III-M is selected from:

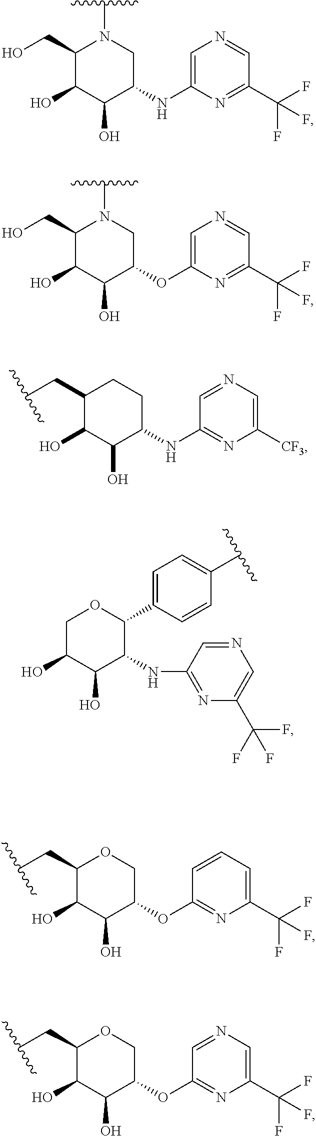 [SMILES: *N1C[C@H](Nc2cncc(C(F)(F)F)n2)[C@@H](O)[C@@H](O)[C@H]1CO ; *N1C[C@H](Oc2cncc(C(F)(F)F)n2)[C@@H](O)[C@@H](O)[C@H]1CO ; *C[C@H]1CC[C@H](Nc2cncc(C(F)(F)F)n2)[C@@H](O)[C@H]1O ; *c1ccc([C@H]2OC[C@H](O)[C@H](O)[C@H]2Nc2cncc(C(F)(F)F)n2)cc1 ; *C[C@H]1OC[C@H](Oc2cccc(C(F)(F)F)n2)[C@@H](O)[C@H]1O ; *C[C@H]1OC[C@H](Oc2cncc(C(F)(F)F)n2)[C@@H](O)[C@H]1O]

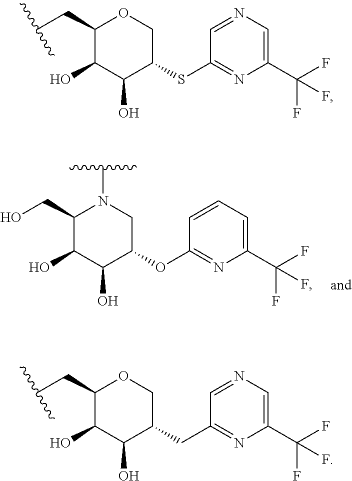 [SMILES: *C[C@H]1OC[C@H](Sc2cncc(C(F)(F)F)n2)[C@@H](O)[C@H]1O ; *N1C[C@H](Oc2cccc(C(F)(F)F)n2)[C@@H](O)[C@@H](O)[C@H]1CO ; *C[C@H]1OC[C@H](Cc2cncc(C(F)(F)F)n2)[C@@H](O)[C@H]1O]

In certain embodiments the ASGPR Binding Ligand used in Formula I-M, II-M, or III-M is selected from:

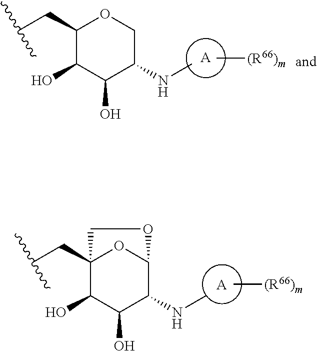

for example an ASGPR Binding Ligand selected from

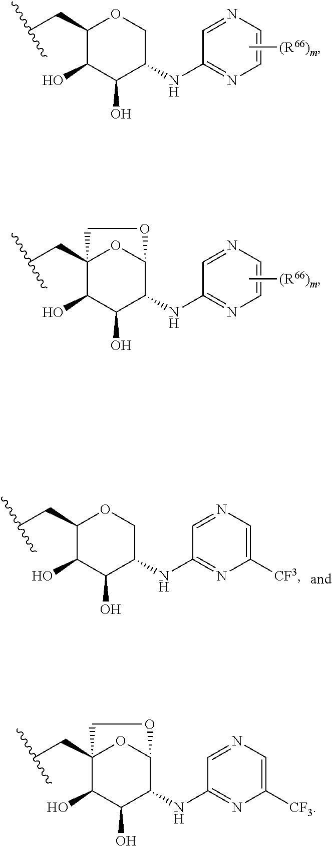

In other embodiments the ASGPR Binding Ligand used in Formula I-M, II-M, or III-M is selected from:

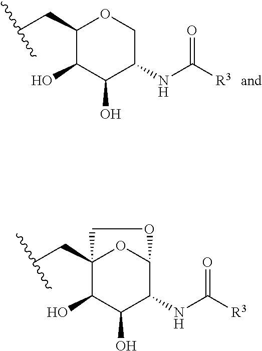

for example an ASGPR Binding Ligand selected from

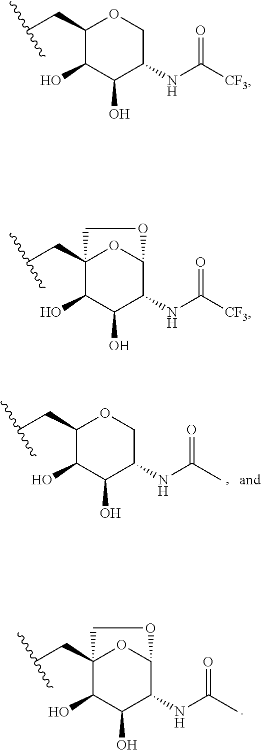 [SMILES: *C[C@H]1OC[C@H](NC(=O)C(F)(F)F)[C@@H](O)[C@H]1O ; *C[C@@]12CO[C@@H](O1)[C@H](NC(=O)C(F)(F)F)[C@@H](O)[C@H]2O ; *C[C@H]1OC[C@H](NC(C)=O)[C@@H](O)[C@H]1O ; *C[C@@]12CO[C@@H](O1)[C@H](NC(C)=O)[C@@H](O)[C@H]2O]

### Phospholipase A₂ Receptor-1 (PLA2R) Autoantibodies

In some embodiments, the Target Extracellular Protein is an autoantibody that binds PLA2R. Phospholipase A2 Receptor-1 (PLA2R) is a major target in autoimmune membranous nephropathy. Membranous nephropathy is one of the leading causes of nephrotic syndrome, with most patients progressing to end-stage renal disease. Current treatment regimes with anti-CD20 antibodies can be ineffective at generating a complete remission. PLA2R is a transmembrane glycoprotein with a cysteine-rich N-terminal extracellular domain. This domain contains the epitope where autoantibodies bind. Reduction of autoantibody levels may provide relief to patients and complete elimination of the autoantibodies could be required to produce a durable remission.

The Protein Data Bank provides the crystal structure of the CTLD7 domain of PLA2R, the region where autoantibodies bind (6JLI; Yu et al. J. Struct. Biol. 207, 295-300). Representative PLA2R autoantibody binding ligands include, but are not limited to,

SEQ ID NO: 88
GIFVIQSESLKKC (Fresquet et al. J. Am. Soc. Nephrol 2015, 26, 302)
SEQ ID NO: 89
SVLTLENCK (Fresquet et al. J. Am. Soc. Nephrol 2015, 26, 302)
SEQ ID NO: 90
SVLTLENC (Brenchley et al. WO2019/081912)
SEQ ID NO: 91
SVLTLDNCK (Brenchley et al. WO2019/081912)
SEQ ID NO: 92
SVLTEENC (Brenchley et al. WO2019/081912)
SEQ ID NO: 93
SVLTEENS (Brenchley et al. WO2019/081912)
SEQ ID NO: 94
SVLTDENC (Brenchley et al. WO2019/081912)
SEQ ID NO: 95
SVLTDENS (Brenchley et al. WO2019/081912)
SEQ ID NO: 96
PIQSESLKK (Brenchley et al. WO2019/081912)
SEQ ID NO: 97
VIDSESLKK (Brenchley et al. WO2019/081912)
SEQ ID NO: 98
PIDSESLKK (Brenchley et al. WO2019/081912)
SEQ ID NO: 99
VIQSESLKK (Brenchley et al. WO2019/081912)
SEQ ID NO: 100
PIESES-PEG-K-PEG-SVLTEENC (Brenchley et al. WO2019/081912)
SEQ ID NO: 101
VIQSES-PEG-K-PEG-SVL TLENC (Brenchley et al. WO2019/081912)
SEQ ID NO: 102
VIQSES-PEG-K-PEG-SVL TEENC (Brenchley et al. WO2019/081912)
SEQ ID NO: 103
PIDDES-PEG-K-PEG-SVLTLENC (Brenchley et al. WO2019/081912)
SEQ ID NO: 104
PIDDES-PEG-KPEG-SVLTEENC (Brenchley et al. WO2019/081912)
SEQ ID NO: 105
VIQSESLKKCKSVLTLENC (Brenchley et al. WO2019/081912)
SEQ ID NO: 106
PIQSESLKKCKSVLTLENC (Brenchley et al. WO2019/081912)
SEQ ID NO: 107
VIESESLKKCKSVLTLENC (Brenchley et al. WO2019/081912)
SEQ ID NO: 108
VIDSESLKKCKSVLTLENC (Brenchley et al. WO2019/081912)
SEQ ID NO: 109
PIESESLKKCKSVLTLENC (Brenchley et al. WO2019/081912)
SEQ ID NO: 110
VIQSESLKKCIQAGKLENC (Brenchley et al. WO2019/081912)
SEQ ID NO: 111
PIQSESLKKCIQAGKLENC (Brenchley et al. WO2019/081912)
SEQ ID NO: 112
VIESESLKKCIQAGKLENC (Brenchley et al. WO2019/081912)
SEQ ID NO: 113
VIDSESLKKCIQAGKLENC (Brenchley et al. WO2019/081912)
SEQ ID NO: 114
PIESESLKKCIQAGKLENC (Brenchley et al. WO2019/081912)
SEQ ID NO: 115
PIQSESLKKCKSVLTLENK (Brenchley et al. WO2019/081912)
SEQ ID NO: 116
VIESESLKKCKSVLTLENK (Brenchley et al. WO2019/081912)
SEQ ID NO: 117
VIDSESLKKCKSVLTLENK (Brenchley et al. WO2019/081912)
SEQ ID NO: 118
PIESESLKKCKSVLTLENK (Brenchley et al. WO2019/081912)
SEQ ID NO: 119
VIQSESLKKCIQAGKLENK (Brenchley et al. WO2019/081912)
SEQ ID NO: 120
PIQSESLKKCIQAGKLENK (Brenchley et al. WO2019/081912)
SEQ ID NO: 121
VIESESLKKCIQAGKLENK (Brenchley et al. WO2019/081912)
SEQ ID NO: 122
VIDSESLKKCIQAGKLENK (Brenchley et al. WO2019/081912)
SEQ ID NO: 123
PIESESLKKCIQAGKLENK (Brenchley et al. WO2019/081912)
SEQ ID NO: 124
PIESESGSVLTLENCK (Brenchley et al. WO2019/081912)
SEQ ID NO: 125
PIESESGGSVLTLENCK (Brenchley et al. WO2019/081912)
SEQ ID NO: 126
PIESESGGGSVLTLENCK (Brenchley et al. WO2019/081912)
SEQ ID NO: 127
PIESESGGGGSVLTLENCK (Brenchley et al. WO2019/081912)
SEQ ID NO: 128
PIESESGGGGGSVLTLENCK (Brenchley et al. WO2019/081912)
SEQ ID NO: 129
VIQSESGSVLTLENCK (Brenchley et al. WO2019/081912)
SEQ ID NO: 130
VIQSESGGSVLTLENCK (Brenchley et al. WO2019/081912)
SEQ ID NO: 131
VIQSESGGGSVLTLENCK (Brenchley et al. WO2019/081912)
SEQ ID NO: 132
VIQSESGGGGSVLTLENCK (Brenchley et al. WO2019/081912)
SEQ ID NO: 133
VIQSESGGGGGSVLTLENCK (Brenchley et al. WO2019/081912)
SEQ ID NO: 134
KGCFVIQSESLKKSIQAGKSVLTLENCK (Brenchley et WO2019/081912)
SEQ ID NO: 135
LKKCIQAGKSVLTLENCKQAN (Brenchley et al. WO2019/081912)
SEQ ID NO: 136
WQDKGIFVIQSESLKKCIQAGK (Brenchley et al. WO2019/081912)
SEQ ID NO: 137
KGIFVIQSESLKKCIQAGKSVLTLENCK (Brenchley et al. WO2019/081912)
SEQ ID NO: 138
GIFVIQSESLKKC (Brenchley et al. WO2015/185949)
SEQ ID NO: 139
WSVLTLENCK (Brenchley et al. WO2015/185949)
SEQ ID NO: 140
WQDKGIFVIQSESLKKCIQAGKSVLTLENCK (Brenchley et al.
WO2015/185949)
SEQ ID NO: 141
YDWIPSSAW (Glee et al., the journal of immunology, 1999,
163: 826-833)
SEQ ID NO: 142
AGAIWQRDW
SEQ ID NO: 143
AGAIWQKDW
SEQ ID NO: 144
VIQSESLK
SEQ ID NO: 145
PIQSESLK
SEQ ID NO: 146
PIESESLK
SEQ ID NO: 147
SVLTEENCK

In certain embodiments a compound is provided of Formula

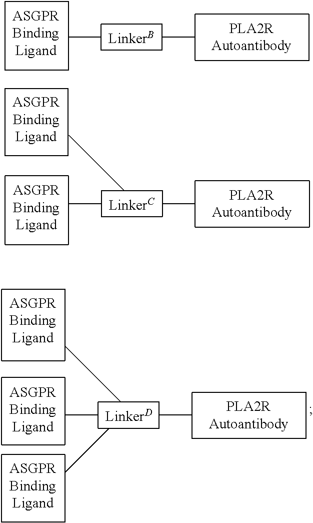

or a pharmaceutically acceptable salt thereof;
wherein
PLA2R Autoantibody is any PLA2R Autoantibody described in WO2019/081912.

In certain embodiments PLA2R Autoantibody is of Formula:

SEQ ID NO: 148:
S-V-L-T-XH1-E-N-XH2;
SEQ ID NO: 149:
XH3-I-XH4-XH5-E-XH6;
SEQ ID NO: 150:
XH1-E-N-XH2-K;
SEQ ID NO: 151:
S-V-L-T-XH1-E-N-C-K;
SEQ ID NO: 152:
XH3-I-XH4-XH5-E-XH6-L-K;

or a peptide of SEQ ID No: 148, 149, 150, 151, or 152 linked via a Linker-B group, in certain embodiments the linked sequences are SEQ ID: 148 and SEQ ID NO: 149 or SEQ ID NO: 148 and SEQ ID NO: 152;
wherein XH1, XH2, XH3, XH4, XH5, and XH6 are independently any natural amino acid or other amino acid described herein;
and wherein the sequence is linked to a Linker described herein at a terminal amine or carboxylic acid.

### Complement C3

In some embodiments the Target Extracellular Protein is complement C3. Complement C3 is one of the major proteins involved in the complement response, a significant factor in both innate and adaptive immunity. Elevated C3 is associated with Paroxysmal nocturnal hemoglobinuria (PNH), immune complex membranoproliferative glomerulonephritis (IC-MPGN), C3 glomerulopathy (C3G), geographic (GA), age-related macular degeneration (AMD), periodontitis, amyotrophic lateral sclerosis (ALS), hematopoietic stem cell transplantation-associated thrombotic microangiopathy (HSCT-TMA), cold agglutinin disease (CAD) and host attack in gene therapies. Reduction of C3 levels may ameliorate some of the symptoms or complications that arise from these inflammatory diseases.

The Protein Data Bank website provides the crystal structure of complement C3, searchable by 2A73 (Janssen, B. J. Nature, 2005, 505-511). Complement C3 bound to a nanobody inhibitor can be found with PDB accession code 6EHG (Jensen, R. K. et al. J Biol Chem, 2018, 293, 6269-6281). Nonlimiting examples of complement C3 binding ligands include

SEQ ID NO: 153
D-Tyr-Ile-[Cys-Val-1MeTrp-Gln-Asp-Trp-Sar-Ala-His-Arg-Cys]-meIle
(Zhang, Y. et al. 2015, Immunobiology, 220, 993-998)
SEQ ID NO: 154
ICVVQDWGHHRCTAGMANLTSHASAI, (Sahu, A. et al. The Journal of
Immunology, 1996, 157, 884-891).
SEQ ID NO: 155
ICVVQDWGHHRCT, (Sahu, A. et al. The Journal of Immunology, 1996,
157, 884-891).
SEQ ID NO: 156
CVVQDWGHHAC (Sahu, A. et al. The Journal of Immunology, 1996, 157,
884-891).
SEQ ID NO: 157
Ac-ICVVQDWGHHRCT-NH2, (Sahu, The Journal of Immunology, 2000,
165, 2491-2499);
SEQ ID NO: 158
CVVQDWGHHRCT-NH₂, (Sahu, The Journal of Immunology, 2000, 165,
2491-2499);
SEQ ID NO: 159
CVVQDWGHHRC-NH₂, (Sahu, The Journal of Immunology, 2000, 165,
2491-2499);
SEQ ID NO: 160
Ac-ICVVGDWGHHRCT-NH2, (Sahu, The Journal of Immunology, 2000,
165, 2491-2499);
SEQ ID NO: 161
Ac-I*CVVQPWGHHRC*T-NH2, (Sahu, The Journal of Immunology,
2000, 165, 2491-2499);
SEQ ID NO: 162
Biotin-KYSSI*CVVQDWGHHRC*T-NH2, (Sahu, The Journal of
Immunology, 2000, 165, 2491-2499);
SEQ ID NO: 163
Ac-I*CVVQDWGHHRC*TAGHMANLTSHASAK-Biotin, (Sahu, The
Journal of Immunology, 2000, 165, 2491-2499);
SEQ ID NO: 164
Ac-ICV(1mW)QDWGAHRCT, (Risitano et al. Blood, 2014, 123, 2094)
SEQ ID NO: 165
yICV(1mW)QDW-Sar-AHRC-mI, (Risitano et al. Blood, 2014, 123, 2094)
SEQ ID NO: 166
PEG-yICV(1mW)QDW-Sar-AHRC-mI (Risitano et al. Blood, 2014, 123,
2094)

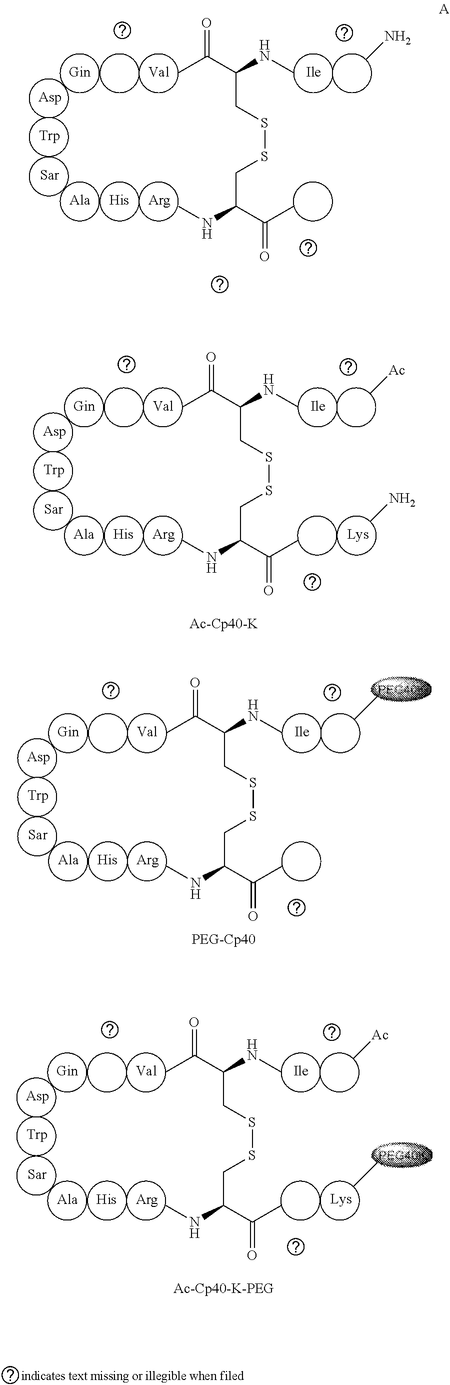

SEQ ID NO: 167
Ac-Ile-[Cys-Val-Trp-Gln-Asp-Trp-Gly-Ala-His-Arg-Cys]-Thr-NH₂ (Qu,
H. et al. Immunobiology (2012) http://dx.doi.org/10.1016/j.imbio.2012.06.003)
SEQ ID NO: 168
Ac-Ile-[Cys-Val-Trp(Me)-Gln-Asp-Trp-Sar-Ala-His-Arg-Cys]-Ile-NH₂
(Qu, H. et al. Immunobiology (2012) http://dx.doi.org/10.1016/j.imbio.2012.06.003)
SEQ ID NO: 169
Ac-Ile-[Cys-Val-Trp(Me)-Gln-Asp-Trp-Sar-Ala-His-Arg-Cys]-mIle-NH₂
(Qu, H. et al. Immunobiology (2012) http://dx.doi.org/10.1016/j.imbio.2012.06.003)
SEQ ID NO: 170
Ac-Ile-[Cys-Val-Trp(Me)-Gln-Asp-Trp-Gly-Ala-His-Arg-Cys]-Thr-NH₂)
(Qu, H. et al. Molecular Immunology, 2011, 48, 481)
SEQ ID NO: 171
Ac-Xaa1-[Cys2-Val3-Xaa4-Gln5-Asp6-Trp7-Gly8-Xaa9-Xaa10-Xaa11-
Cys12]-Thr13-NH₂ (Mallik et al. J. Med. Chem., 2005, 48, 274-286)
SEQ ID NO: 172
Ac-I[CVVQDWGHHRC]T-NH₂ (Mallik et al. J. Med. Chem., 2005, 48,
274-286)
SEQ ID NO: 173
Ac-I[CVVQDWGAHRC]T-NH₂ (Mallik et al. J. Med. Chem., 2005, 48,
274-286)
SEQ ID NO: 174
Ac-I[CVTQDWGHHRC]T-NH₂ (Mallik et al. J. Med. Chem., 2005, 48,
274-286)
SEQ ID NO: 175
Ac-I[CVSQDWGHHRC]T-NH₂ (Mallik et al. J. Med. Chem., 2005, 48,
274-286)
SEQ ID NO: 176
Ac-I[CVHQDWGHHRC]T-NH₂, (Mallik et al. J. Med. Chem., 2005, 48,
274-286)
SEQ ID NO: 177
Ac-I[CVFQDWGHHRC]T-NH₂, (Mallik et al. J. Med. Chem., 2005, 48,
274-286)
SEQ ID NO: 178
Ac-I[CVYQDWGAHRC]T-NH₂, (Mallik et al. J. Med. Chem., 2005, 48,
274-286)
SEQ ID NO: 179
Ac-I[CVWQDWGWHRC]T-NH₂, (Mallik et al. J. Med. Chem., 2005, 48,
274-286)
SEQ ID NO: 180
Ac-I[CVWQDWGHHRC]T-NH₂, (Mallik et al. J. Med. Chem., 2005, 48,
274-286)
SEQ ID NO: 181
Ac-I[CVWQDWGAHRC]T, (Mallik et al. J. Med. Chem., 2005, 48, 274-
286)
SEQ ID NO: 182
Ac-I[CVWQDWGAHRC]T-NH₂, (Mallik et al. J. Med. Chem., 2005, 48,
274-286)
SEQ ID NO: 183
Ac-I[CVWQDWGAdHRC]T, (Mallik et al. J. Med. Chem., 2005, 48, 274-
286)
SEQ ID NO: 184
Ac-I[CVWQDWGdAHRC]T, (Mallik et al. J. Med. Chem., 2005, 48, 274-
286)
SEQ ID NO: 185
Ac-dI[CVWQDWGAHRC]T, (Mallik et al. J. Med. Chem., 2005, 48, 274-
286)
SEQ ID NO: 186
Ac-I[CVWQDWGAHRC]dT, (Mallik et al. J. Med. Chem., 2005, 48, 274-
286)
SEQ ID NO: 187
Ac-I[CVWQDWGAHRC]T-NH₂, (Lopez de Victoria, A. et al. Chem Biol
Drug Des 2011, 77, 431-440)
SEQ ID NO: 188
W[CVWQDWGTNRC]W-NH₂, (Lopez de Victoria, A. et al. Chem Biol
Drug Des 2011, 77, 431-440)
SEQ ID NO: 189
Ac-D[CVWQDWGTNKC]W-NH₂, (Lopez de Victoria, A. et al. Chem Biol
Drug Des 2011, 77, 431-440)
SEQ ID NO: 190
Q[CVWQDWGQNQC]W-NH₂, (Lopez de Victoria, A. et al. Chem Biol
Drug Des 2011, 77, 431-440)
SEQ ID NO: 191
Ac-I[CVWQDWGAHRC]W-NH₂, (Lopez de Victoria, A. et al. Chem Biol
Drug Des 2011, 77, 431-440)
SEQ ID NO: 192
Ac-W[CVWQDWGAHRC]T-NH₂, (Lopez de Victoria, A. et al. Chem Biol
Drug Des 2011, 77, 431-440)
SEQ ID NO: 193
Ac-W[CVWQDWGAHRC]W-NH₂, (Lopez de Victoria, A. et al. Chem
Biol Drug Des 2011, 77, 431-440)
SEQ ID NO: 194
Ac-Ile-[Ala-Val-Trp-Gln-Asp-Trp-Gly-Ala-His-Arg-Hcy]-Thr-NH₂,
(Knerr, P. et al. ACS Chem. Biol., 2011, 6, 753-760)
SEQ ID NO: 195
Ac-Ile-[Cys-Val-Trp-Gln-Asp-Trp-Gly-Ala-His-Arg-Cys]-(NMeIle)-NH₂,
(Knerr, P. et al. ACS Chem. Biol., 2011, 6, 753-760)
SEQ ID NO: 196
Ac-Ile-[Ala-Val-Trp-Gln-Asp-Trp-Gly-Ala-His-Arg-Hcy]-(NMeIle)-NH₂,
(Knerr, P. et al. ACS Chem. Biol., 2011, 6, 753-760)
SEQ ID NO: 197
Ac-ICV(5fW)QDWGAHRCT-NH2, (Katragadda et al. J. Med. Chem.
2006, 49, 4616-4622).
SEQ ID NO: 198
Ac-ICV(5MeW)QDWGAHRCT-NH2, (Katragadda et al. J. Med. Chem.
2006, 49, 4616-4622).
SEQ ID NO: 199
Ac-ICV(2Nal)QDWGAHRCT-NH2, (Katragadda et al. J. Med. Chem.
2006, 49, 4616-4622).
SEQ ID NO: 200
Ac-ICVWQD(5fW)GAHRCT-NH2, (Katragadda et al. J. Med. Chem.
2006, 49, 4616-4622).
SEQ ID NO: 201
Ac-ICVWQD(5MeW)GAHRCT-NH2, (Katragadda et al. J. Med. Chem.
2006, 49, 4616-4622).
SEQ ID NO: 202
Ac-ICVWQD(1MeW)GAHRCT-NH2, (Katragadda et al. J. Med. Chem.
2006, 49, 4616-4622).
SEQ ID NO: 203
Ac-ICVYQDWGAHRCT-CONH2, (WO 2021/007, 111)
SEQ ID NO: 204
Ac-ICVWQDWGAHRCT-COOH, (WO 2021/007, 111)
SEQ ID NO: 205
Ac-ICVWQDWGAHRCT-CONH2, (WO 2021/007, 111)
SEQ ID NO: 206
Ac-ICVWQDWGAHRCdT-COOH, (WO 2021/007, 111)
SEQ ID NO: 207
Ac-ICV(2-Nal)QDWGAHRCT-CONH2, (WO 2021/007, 111)
SEQ ID NO: 208
Ac-ICV(2-Nal)QDWGAHRCT-COOH, (WO 2021/007, 111)
SEQ ID NO: 209
Ac-ICV(1-Nal)QDWGAHRCT-COOH, (WO 2021/007, 111)
SEQ ID NO: 210
Ac-ICV(2-lal)QDWGAHRCT-CONH2, (WO 2021/007, 111)
SEQ ID NO: 211
Ac-ICV(2-lal)QDWGAHRCT-COOH, (WO 2021/007, 111)
SEQ ID NO: 212
Ac-ICVDhtQDWGAHRCT-COOH, (WO 2021/007, 111)
SEQ ID NO: 213
Ac-ICV(Boa)QDWGAHRCT-COOH, (WO 2021/007, 111)
SEQ ID NO: 214
Ac-ICV(Bpa)QDWGAHRCT-CONH2, (WO 2021/007, 111)
SEQ ID NO: 215
Ac-ICV(Bta)QDWGAHRCT-COOH, (WO 2021/007, 111)
SEQ ID NO: 216
Ac-ICV(Bta)QDWGAHRCT-CONH2, (WO 2021/007, 111)
SEQ ID NO: 217
Ac-ICVWQDWG(2-Abu)HRCT-CONH2, (WO 2021/007, 111)
SEQ ID NO: 218
H-GICVWQDWGAHRCTAN-COOH, (WO 2021/007, 111)
SEQ ID NO: 219
Ac-ICV(5fW)QDWGAHRCT-CONH2, (WO 2021/007, 111)
SEQ ID NO: 220
Ac-ICV(5-methvl-W)QDWGAHRCT CONH2, (WO 2021/007, 111)
SEQ ID NO: 221
Ac-ICV(1-methvl-W)QDWGAHRCT-CONH2, (WO 2021/007, 111)
SEQ ID NO: 222
Ac-ICVWQD(5fW)GAHRCT-CONH2, (WO 2021/007, 111)
SEQ ID NO: 223
Ac-ICV(5fW)QD(5fW)GAHRCT-CONH2, (WO 2021/007, 111)
SEQ ID NO: 224
Ac-ICV(5-methyl-W)QD(5fW)GAHRCT-CONH2, (WO 2021/007, 111)
SEQ ID NO: 225
Ac-ICV(1-methyl-W)QD(5fW)GAHRCT-CONH2, (WO 2021/007, 111)
SEQ ID NO: 226
H-GICV(6fW)QD(6fW)GAHRCTN-COOH, (WO 2021/007, 111)
SEQ ID NO: 227
Ac-ICV(1-formvl-W)QDWGAHRCT-CONH2, (WO 2021/007, 111)
SEQ ID NO: 228
Ac-ICV(1-methyoxy-W)QDWGAHRCT-CONH2, (WO 2021/007, 111)
SEQ ID NO: 229
H-GICV(5fW)QD(5fW)GAHRCTN-COOH, (WO 2021/007, 111)

In certain embodiments the complement C3 targeting ligand is

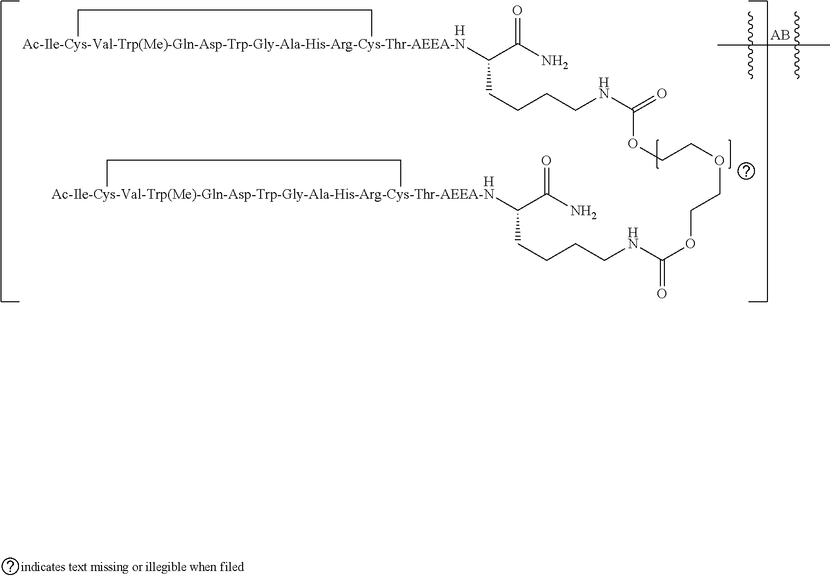

In certain embodiments the complement C3 Targeting Ligand is selected from:

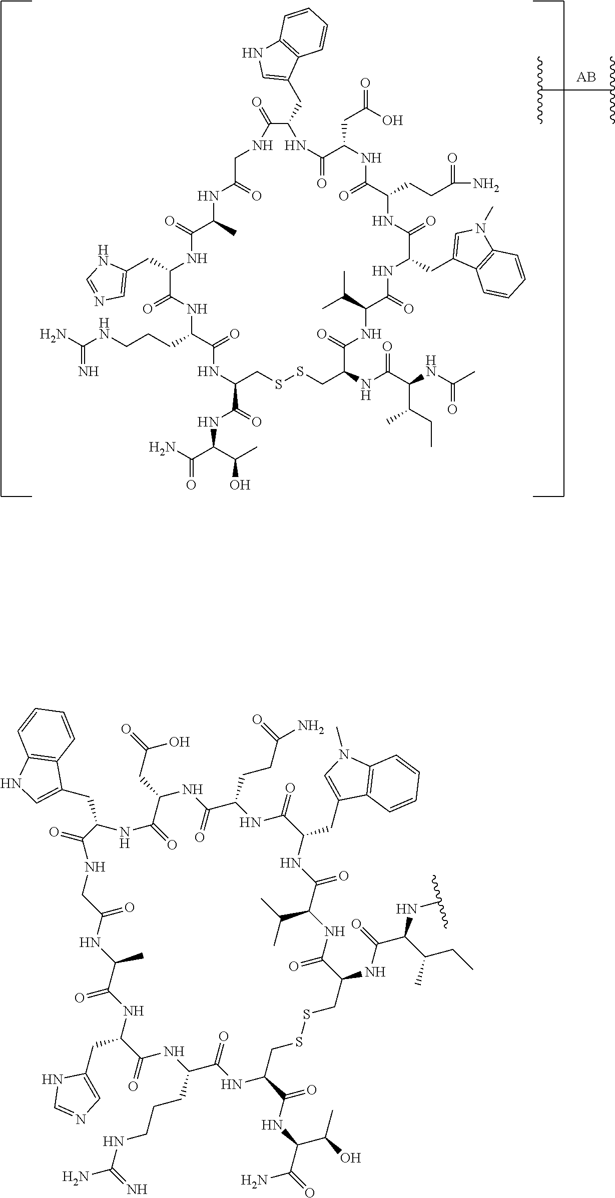

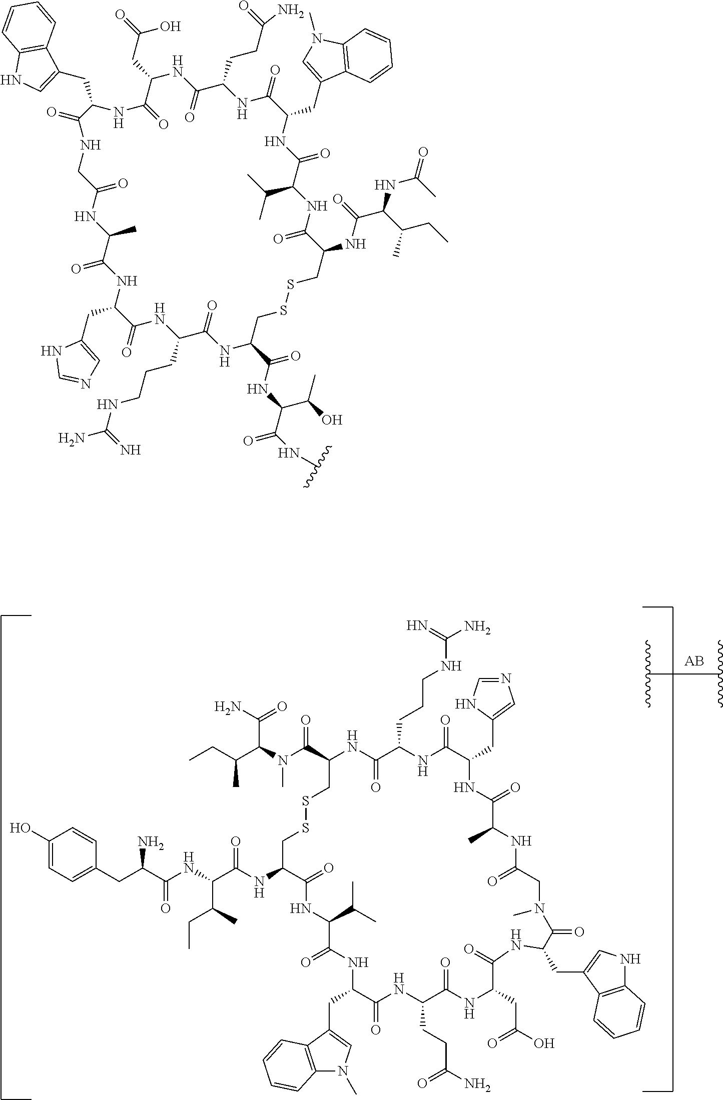

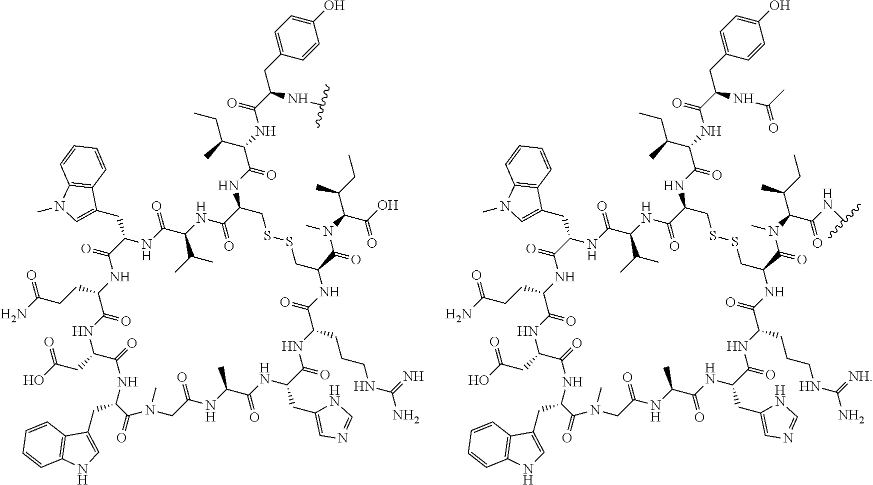

### Complement C1q

In some embodiments, the Target Extracellular Protein is Complement C1q. The complement system is part of the innate immune system and clears apoptotic cells and pathogens. Activation of this pathway begins with binding the C1 complex to an immunoglobulin that has bound to an antigen. The C1 complex consists of C1q and a tetramer of proteases (C1r and C1s). C1q mediates the binding of complement to IgG or IgM. Following the binding event, the proteases are activated, and they cleave C4 which sets off the remainder of the pathway that ends in opsonization. Overactivity of this pathway can lead to a number of inflammatory pathologies including allograft rejection, neuromyelitis optica, generalized myasthenia gravis, and cold agglutinin disease. Degradation of C1q may reduce the symptoms associated with these inflammatory diseases.

The Protein Data Bank website provides the crystal structure of Complement C1q searchable by 2JG9 (Paidassi, H. et al., J. Immunol, 2008, 180, 2329-2338), 1PK6 (Gaboriaud, C., J. Biol. Chem, 2003, (278) 46974-46982), 5HZF (Moreau, C. et al., Front. Immunol, 2016, (7) 79) 2WNV and 2WNU (Garlatti, V. et. al., J. Immunol. 2010, (185), 808). Also provided on the PDB website is the structure of complement C1q with a ligand bound, searchable by 6Z67 (Laursen, N. et al. Front. Immunol., 2020, (11), 1504)

Nonlimiting examples of complement C1q binding ligands include

SEQ ID NO: 230
Ac-Ala-Glu-Ala-Lys-Ala-Lys-Ala-CONH2 (WO 88/07054)
SEQ ID NO: 231
IALILEPICCQERAA (Sharp, J. A. et al. PLOS ONE 10(7), e0132446)
SEQ ID NO: 232
IALILEPICCQERAA-dPEG24 (Sharp, J. A. et al. PLOS ONE 10(7),
e0132446)
SEQ ID NO: 233
dPEG24-IALILEPICCQERAA (Sharp, J. A. et al. PLOS ONE 10(7),
e0132446)
SEQ ID NO: 234
RALILEPICCQERAA (Sharp, J. A. et al. PLOS ONE 10(7), e0132446)
SEQ ID NO: 235
IRLILEPICCQERAA (Sharp, J. A. et al. PLOS ONE 10(7), e0132446)
SEQ ID NO: 236
IARILEPICCQERAA (Sharp, J. A. et al. PLOS ONE 10(7), e0132446)
SEQ ID NO: 237
IALIREPICCQERAA (Sharp, J. A. et al. PLOS ONE 10(7), e0132446)
SEQ ID NO: 238
IALILEPICCRERAA (Sharp, J. A. et al. PLOS ONE 10(7), e0132446)
SEQ ID NO: 239
IALILEPICCORRAA (Sharp, J. A. et al. PLOS ONE 10(7), e0132446)
SEQ ID NO: 240
IELILEPICCQERAA (Sharp, J. A. et al. PLOS ONE 10(7), e0132446)
SEQ ID NO: 241
IAEILEPICCQERAA (Sharp, J. A. et al. PLOS ONE 10(7), e0132446)
SEQ ID NO: 242
IALILEPICCQEEAA (Sharp, J. A. et al. PLOS ONE 10(7), e0132446)
SEQ ID NO: 243
IALILEPICCQEREA (Sharp, J. A. et al. PLOS ONE 10(7), e0132446)
SEQ ID NO: 244
IALILEEICCQERAA (Sharp, J. A. et al. PLOS ONE 10(7), e0132446)
SEQ ID NO: 245
IALILEPECCQERAA (Sharp, J. A. et al. PLOS ONE 10(7), e0132446)
SEQ ID NO: 246
PAICQRATATLGTVGSNTSGTTAIEACILL (Sharp, J. A. et al. Frontiers
in Immunology (2014) 5, 406)
SEQ ID NO: 247
CEGPFGPRHDLTFCW (Roos, A. et al. The Journal of Immunology,
2001, 167, 7052)
SEQ ID NO: 248
XbEGPFGPRHDLTFCW (Roos, A. et al. The Journal of Immunology,
2001, 167, 7052)
SEQ ID NO: 249
QYYPFSX (Messmer B.T. et al. Molecular Immunology, 2000, 37, 343)
SEQ ID NO: 250
NPFNLAR (Messmer B.T. et al. Molecular Immunology, 2000, 37, 343)
SEQ ID NO: 251
QLQDMTSSPFWL (Messmer B.T. et al. Molecular Immunology, 2000,
37, 343)
SEQ ID NO: 252
NPFVIGRWHPPH (Messmer B.T. et al. Molecular Immunology, 2000, 37,
343)
SEQ ID NO: 253
SLAKFLNPFLYR (Messmer B.T. et al. Molecular Immunology, 2000, 37,
343)
SEQ ID NO: 254
ASTPRFEPFQLD (Messmer B.T. et al. Molecular Immunology, 2000, 37,
343)
SEQ ID NO: 255
SLHSQPYSPFML (Messmer B.T. et al. Molecular Immunology, 2000, 37,
343)
SEQ ID NO: 256
NILSSWSSPFVF (Messmer B.T. et al. Molecular Immunology, 2000, 37,
343)
SEQ ID NO: 257
NLPSSWTNPFYL (Messmer B.T. et al. Molecular Immunology, 2000, 37,
343)
SEQ ID NO: 258
SPFMLHP (Messmer B.T. et al. Molecular Immunology, 2000, 37, 343)
SEQ ID NO: 259
PSPFMLT (Messmer B.T. et al. Molecular Immunology, 2000, 37, 343)
SEQ ID NO: 260
IGPFHLH (Messmer B.T. et al. Molecular Immunology, 2000, 37, 343)
SEQ ID NO: 261
TNPFMLN (Messmer B.T. et al. Molecular Immunology, 2000, 37, 343)
SEQ ID NO: 262
NTTFLYP (Messmer B.T. et al. Molecular Immunology, 2000, 37, 343)
SEQ ID NO: 263
SHYTQYL (Messmer B.T. et al. Molecular Immunology, 2000, 37, 343)
SEQ ID NO: 264
NHHPNYW (Messmer B.T. et al. Molecular Immunology, 2000, 37, 343)
SEQ ID NO: 265
VHYPLSW (Messmer B.T. et al. Molecular Immunology, 2000, 37, 343)
SEQ ID NO: 266
HHLKYSDTSPPI (Messmer B.T. et al. Molecular Immunology, 2000, 37,
343)
SEQ ID NO: 267
SHMHERWDTSPPI (Messmer B.T. et al. Molecular Immunology, 2000,
37, 343)
SEQ ID NO: 268
SHMHERWDTSYQ (Messmer B.T. et al. Molecular Immunology, 2000,
37, 343)
SEQ ID NO: 269
SHIHSNAAWRIT (Messmer B.T. et al. Molecular Immunology, 2000, 37,
343)
SEQ ID NO: 270
WHYPHWQ (Messmer B.T. et al. Molecular Immunology, 2000, 37, 343)
SEQ ID NO: 271
SHYLYTQ (Messmer B.T. et al. Molecular Immunology, 2000, 37, 343)
SEQ ID NO: 272
AHYSFTQ (Messmer B.T. et al. Molecular Immunology, 2000, 37, 343)
SEQ ID NO: 273
THYPTFY (Messmer B.T. et al. Molecular Immunology, 2000, 37, 343)
SEQ ID NO: 274
EHNTSFW (Messmer B.T. et al. Molecular Immunology, 2000, 37, 343)
SEQ ID NO: 275
NHYKLTW (Messmer B.T. et al. Molecular Immunology, 2000, 37, 343)
SEQ ID NO: 276
NHSPYFQ (Messmer B.T. et al. Molecular Immunology, 2000, 37, 343)
SEQ ID NO: 277
SHYQHYQ (Messmer B.T. et al. Molecular Immunology, 2000, 37, 343)
SEQ ID NO: 278
PAICQRATATLGTVGSNTSGTTEIEACILL (Gronemus, J.Q. et al.
Molecular immunology, 2010, 48, 305)
SEQ ID NO: 279
WLGLGGGYGW (Lauvrak V., Biol. Chem. 1997, 378, 1509)
SEQ ID NO: 280
FYGPFFLNDSLRGIW (Lauvrak V., Biol. Chem. 1997, 378, 1509)
SEQ ID NO: 281
LRFLNPFSLDGSGFW (Lauvrak V., Biol. Chem. 1997, 378, 1509)
SEQ ID NO: 282
HSPFCLGVLECFGLV (Lauvrak V., Biol. Chem. 1997, 378, 1509)
SEQ ID NO: 283
TCGAFYLYHDPFICG (Lauvrak V., Biol. Chem. 1997, 378, 1509)
SEQ ID NO: 284
MQHCLASHELYLPWC (Lauvrak V., Biol. Chem. 1997, 378, 1509)
SEQ ID NO: 285
FFVFGSGDAFAFSDM (Lauvrak V., Biol. Chem. 1997, 378, 1509)
SEQ ID NO: 286
PCVIIDTGSSRWCYL (Lauvrak V., Biol. Chem. 1997, 378, 1509)
SEQ ID NO: 287
HSPFCLGVLECFGLV (Lauvrak V., Biol. Chem. 1997, 378, 1509)
SEQ ID NO: 288
HAAFEPRGDVRHTLL (Lauvrak V., Biol. Chem. 1997, 378, 1509)
SEQ ID NO: 289
CRWDGSWGEVRC (Lauvrak V., Biol. Chem. 1997, 378, 1509)
SEQ ID NO: 290
CYWVGTWGEAVC (Lauvrak V., Biol. Chem. 1997, 378, 1509)
SEQ ID NO: 291
RWFPCPNKEGCCSISV (Lauvrak V., Biol. Chem. 1997, 378, 1509)
SEQ ID NO: 292
RSTYCNKNKDSCHIPE (Lauvrak V., Biol. Chem. 1997, 378, 1509)
SEQ ID NO: 293
QPPQCIKDGGFVICRV (Lauvrak V., Biol. Chem. 1997, 378, 1509)
SEQ ID NO: 294
KGKKCKPEEHPCNEPM (Lauvrak V., Biol. Chem. 1997, 378, 1509)
SEQ ID NO: 295
NKMTCSDDGKLCWEHL (Lauvrak V., Biol. Chem. 1997, 378, 1509)
SEQ ID NO: 296
PLGRPCPTCPLAPS (Lauvrak V., Biol. Chem. 1997, 378, 1509)
SEQ ID NO: 297
QRMRPCPSCPLAPW (Lauvrak V., Biol. Chem. 1997, 378, 1509)
SEQ ID NO: 298
WPSRPCPSCPEVPP (Lauvrak V., Biol. Chem. 1997, 378, 1509)
SEQ ID NO: 299
SCTKDCPTCPLVPV (Lauvrak V., Biol. Chem. 1997, 378, 1509)
C1qNb75 Nanobody (Laursen, N. S. et al. Frontiers in Immunology, 2020, 11, 1504)
SEQ ID NO: 300
IALILEPICCQERAA (U.S. Pat. No. 8,906,845)
SEQ ID NO: 301
PAICQRATATLGTVGSNTSGTTEIEACILL (U.S. Pat. No. 8,906,845)
SEQ ID NO: 302
PAIAQRATATLGTVGSNTSGTTEIEACILL (U.S. Pat. No. 8,906,845)
SEQ ID NO: 303
PAICQRATATLGTVGSNTSGTTEIEAAILL (U.S. Pat. No. 8,906,845)
SEQ ID NO: 304
PAICQRATATLGTVGSNTSGTTAIEACILL (U.S. Pat. No. 8,906,845)
SEQ ID NO: 305
PAICQRATATLGTVGSNTSGTTEIAACILL (U.S. Pat. No. 8,906,845)
SEQ ID NO: 306
PAICQRAEIEACILL (U.S. Pat. No. 8,906,845)
SEQ ID NO: 307
PAICQRAEIEACILL (U.S. Pat. No. 8,906,845)
SEQ ID NO: 308
PAIAQRAEIEAAILL (U.S. Pat. No. 8,906,845)
SEQ ID NO: 309
IALILEPICCQERAA (U.S. Pat. No. 8,906,845)
SEQ ID NO: 310
PAICQRATATLGTNTSGTTEIEACILL (U.S. Pat. No. 8,906,845)
SEQ ID NO: 311
PAICQRATATLSGTTEIEACILL (U.S. Pat. No. 8,906,845)
SEQ ID NO: 312
PAICQRATATTEIEACILL (U.S. Pat. No. 8,906,845)
SEQ ID NO: 313
PAICQRAEIEACILL (U.S. Pat. No. 8,906,845)
SEQ ID NO: 314
AICQRATATLGTVGSNTSGTTEIEACILL (U.S. Pat. No. 8,906,845)
SEQ ID NO: 315
ICQRATATLGTVGSNTSGTTEIEACILL (U.S. Pat. No. 8,906,845)
SEQ ID NO: 316
CQRATATLGTVGSNTSGTTEIEACILL (U.S. Pat. No. 8,906,845)
SEQ ID NO: 317
PAICQRATATLGTVGSNTSGTTEIEACIL (U.S. Pat. No. 8,906,845)
SEQ ID NO: 318
PAICQRATATLGTVGSNTSGTTEIEACI (U.S. Pat. No. 8,906,845)
SEQ ID NO: 319
PAICQRATATLGTVGSNTSGTTEIEAC (U.S. Pat. No. 8,906,845)
SEQ ID NO: 320
Ac-IALILEPICCQERAA (U.S. Pat. No. 8,906,845)
SEQ ID NO: 321
Ac-PAICQRATATLGTVGSNTSGTTEIEACILL (U.S. Pat. No. 8,906,845)
SEQ ID NO: 322
Ac-PAIAQRATATLGTVGSNTSGTTEIEACILL (U.S. Pat. No. 8,906,845)
SEQ ID NO: 323
Ac-PAICQRATATLGTVGSNTSGTTEIEAAILL (U.S. Pat. No. 8,906,845)
SEQ ID NO: 324
Ac-PAICQRATATLGTVGSNTSGTTAIEACILL (U.S. Pat. No. 8,906,845)
SEQ ID NO: 325
Ac-PAICQRATATLGTVGSNTSGTTEIAACILL (U.S. Pat. No. 8,906,845)
SEQ ID NO: 326
Ac-PAICQRAEIEACILL (U.S. Pat. No. 8,906,845)
SEQ ID NO: 327
Ac-PAICQRAEIEACILL (U.S. Pat. No. 8,906,845)
SEQ ID NO: 328
Ac-PAIAQRAEIEAAILL (U.S. Pat. No. 8,906,845)
SEQ ID NO: 329
Ac-IALILEPICCQERAA (U.S. Pat. No. 8,906,845)
SEQ ID NO: 330
Ac-PAICQRATATLGTNTSGTTEIEACILL (U.S. Pat. No. 8,906,845)
SEQ ID NO: 331
Ac-PAICQRATATLSGTTEIEACILL (U.S. Pat. No. 8,906,845)
SEQ ID NO: 332
Ac-PAICQRATATTEIEACILL (U.S. Pat. No. 8,906,845)
SEQ ID NO: 333
Ac-PAICQRAEIEACILL (U.S. Pat. No. 8,906,845)
SEQ ID NO: 334
Ac-AICQRATATLGTVGSNTSGTTEIEACILL (U.S. Pat. No. 8,906,845)
SEQ ID NO: 335
Ac-ICQRATATLGTVGSNTSGTTEIEACILL (U.S. Pat. No. 8,906,845)
SEQ ID NO: 336
Ac-CQRATATLGTVGSNTSGTTEIEACILL (U.S. Pat. No. 8,906,845)
SEQ ID NO: 337
Ac-PAICQRATATLGTVGSNTSGTTEIEACIL (U.S. Pat. No. 8,906,845)
SEQ ID NO: 338
Ac-PAICQRATATLGTVGSNTSGTTEIEACI (U.S. Pat. No. 8,906,845)
SEQ ID NO: 339
Ac-PAICQRATATLGTVGSNTSGTTEIEAC (U.S. Pat. No. 8,906,845)

In certain embodiments the Linker is bound through the C-terminus of the amino acid sequence for example

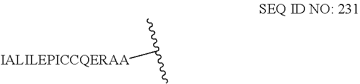

In certain embodiments the Linker is bound to the N-terminus for example

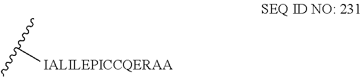

### IL-17

In some embodiments, the Target Extracellular Protein is human interleukin-17 (IL-17) (UniProtKB—Q16552 (IL17_HUMAN)). Interleukin-17 is a 35 kDa homodimeric glycoprotein and is an important cytokine for the inflammatory response. IL-17 is secreted by a distinct class of Helper T cells (known as Th17 cells) which mediates tissue inflammation. A characteristic effect of IL-17 production is the expansion of neutrophils, and in healthy tissue it is responsible for neutrophil homeostasis. IL-17 has been implicated as a major factor in psoriasis as well as other autoimmune diseases. Other diseases where IL-17 therapies may be of benefit include but are not limited to asthma, rheumatoid arthritis, psoriatic arthritis, Crohn's disease, and inflammatory bowel disease. Inflammation caused by IL-17 has been shown to hamper recovery post-stroke.

The Protein Data Bank website provides the crystal structure of IL-17, searchable by 4NUX (Zhang, B. et al. (2014) Acta Crystallogr D Biol Crystallogr 70: 1476-1483), 4HSA (Liu, S. et al. (2013) Nat Commun 4: 1888-1888), 4QHU (unpublished), 6WIR (Lieu, R. et al. (2020) PLoS One 15: e0232311-e0232311), 5VB9 (Ting, J. P. et al. (2018) PLoS One 13: e0190850-e0190850), 4NUX (Zhang, et al. (2014) Acta Crystallogr D Biol Crystallogr 70: 1476-1483), 3JVF (Ely, L. K. et al. (2009) Nat Immunol 10: 1245-1251), 5N9B (unpublished), 2VXS (Gerhardt, S. et al. (2009) J Mol Biol 394: 905).

Non-limiting examples of IL-17 Targeting Ligands can be found in, for example, WO2012101263A1, WO2020163554A1, WO2021055376A1, WO2020146194A1, WO2020127685A1, US20150005319, WO2014066726A2, WO2019223718A1, WO2020135872A1, WO2020146194A1, WO2021027721A1, WO2021027724, WO2021027729A1, WO2021067191A1, CN104069102A, CN105601617B, CN108299256B, Liu et al. “Binding site elucidation and structure guided design of macrocyclic IL-17A antagonists” 2016, Scientific Reports, 6:30859., Liu et al. “Inhibiting complex IL-17AA and IL-17RA interactions with a linear peptide” 2016, Scientific Reports 6:26071. Wang, W. et al. “Artificial macrocycles as IL-17A/IL-17RA antagonists”. Med. Chem. Comm. 2018, 9, 22. Liu, C. et al. “The flavonoid cyanidin blocks binding of the cytokine interleukin-17A to the IL-17RA subunit to alleviate inflammation in vivo” Science Signaling 10, eaaf8823 (2017).

Additional binding ligands include

SEQ ID NO: 340
IVVTAPADLWDWIRA (Liu et al. 2016, Scientific Reports 6: 26071)
SEQ ID NO: 341
ITVTMPADLWDWIRA (Liu et al. 2016, Scientific Reports 6: 26071)
SEQ ID NO: 342
IVVTIPADLWDWIRA (Liu et al. 2016, Scientific Reports 6: 26071)
SEQ ID NO: 343
IVVTLPADLWDWIRA (Liu et al. 2016, Scientific Reports 6: 26071)
SEQ ID NO: 344
IVVTVPADLWDWIRA (Liu et al. 2016, Scientific Reports 6: 26071)
SEQ ID NO: 345
IVVTMPADLWDWIMA (Liu et al. 2016, Scientific Reports 6: 26071)
SEQ ID NO: 346
IVVTMPADLWDWINA (Liu et al. 2016, Scientific Reports 6: 26071)
SEQ ID NO: 347
IVVTMPADLWDWIQA (Liu et al. 2016, Scientific Reports 6: 26071)
SEQ ID NO: 348
IHVTIPADLWDWINK (Liu et al. 2016, Scientific Reports 6: 26071)
SEQ ID NO: 349
IHVTIPADLWDWIN (Liu et al. 2016, Scientific Reports 6: 26071)

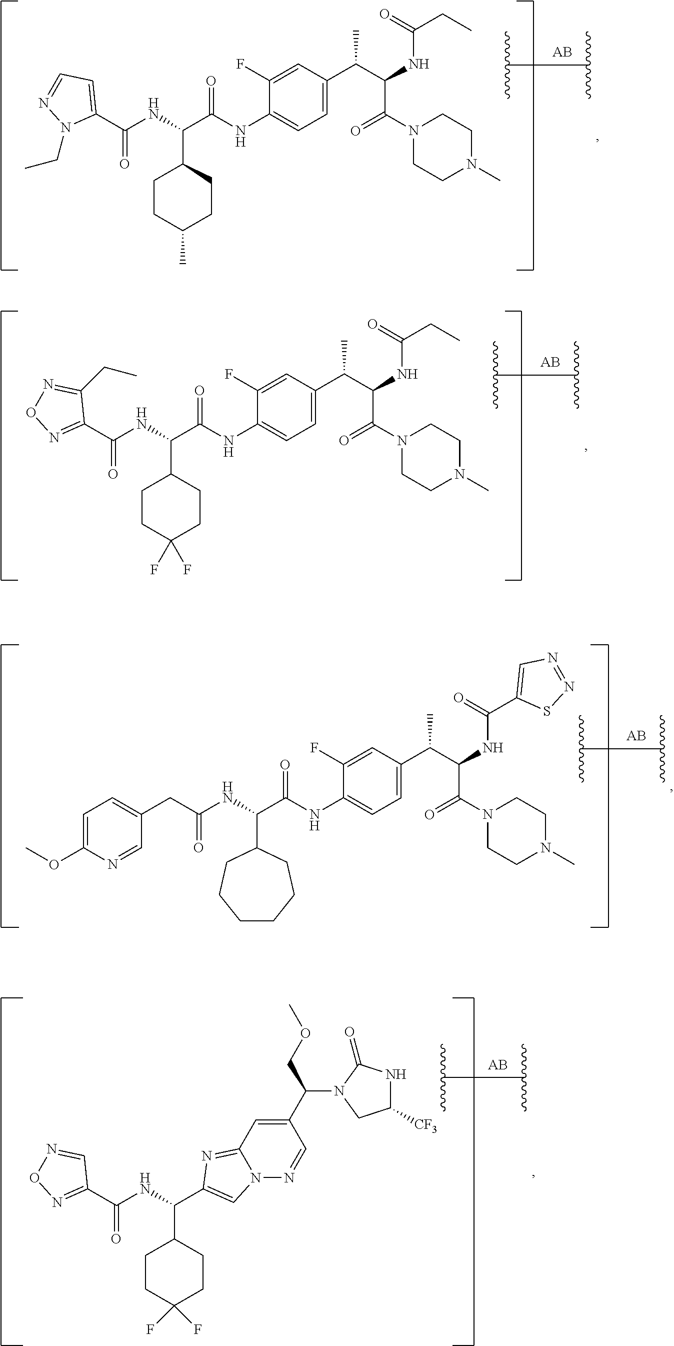

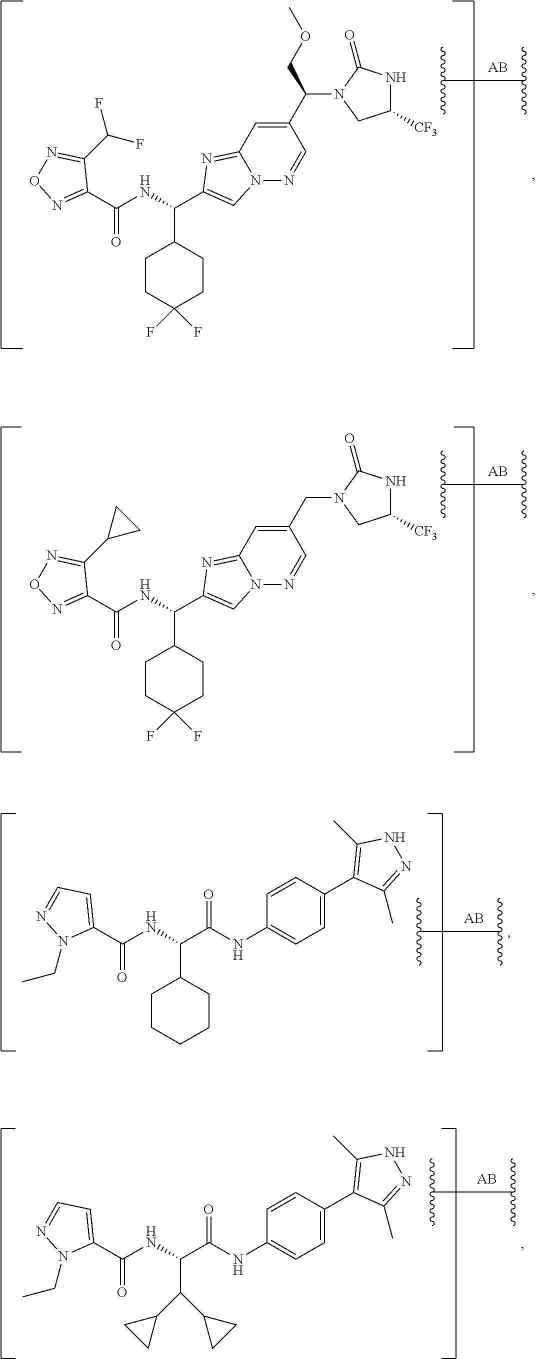

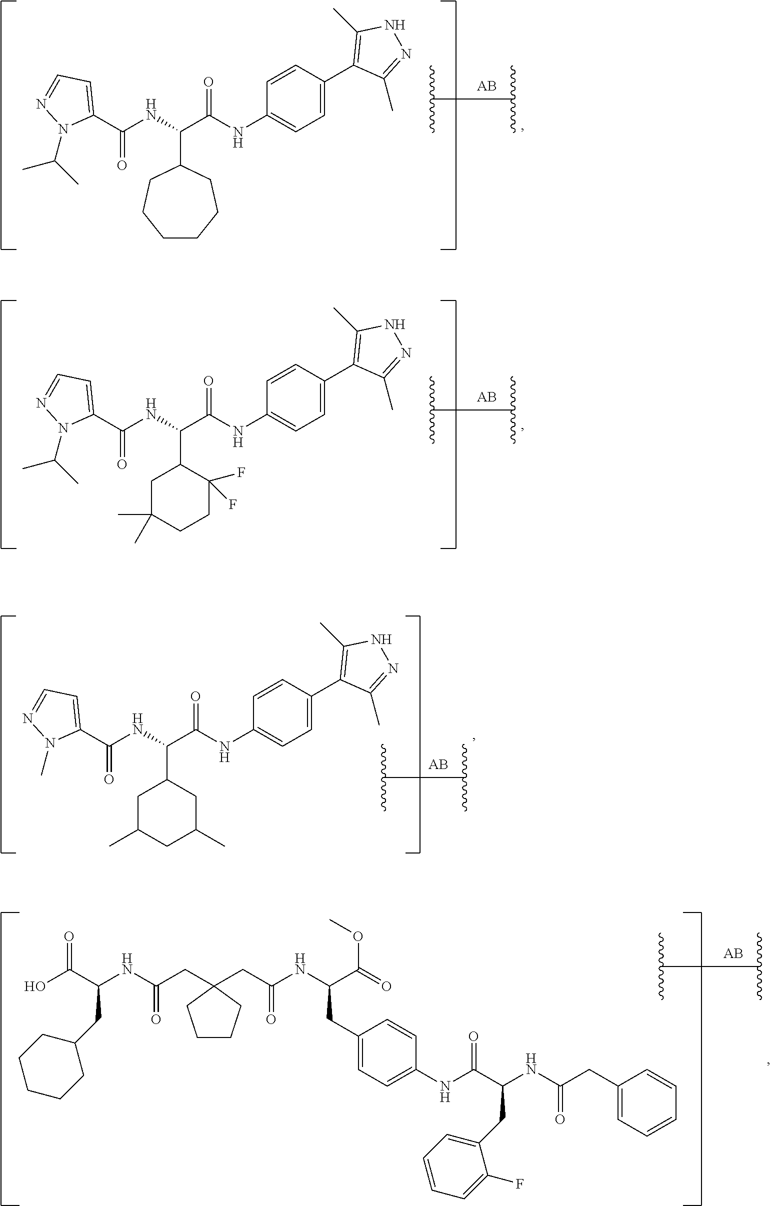

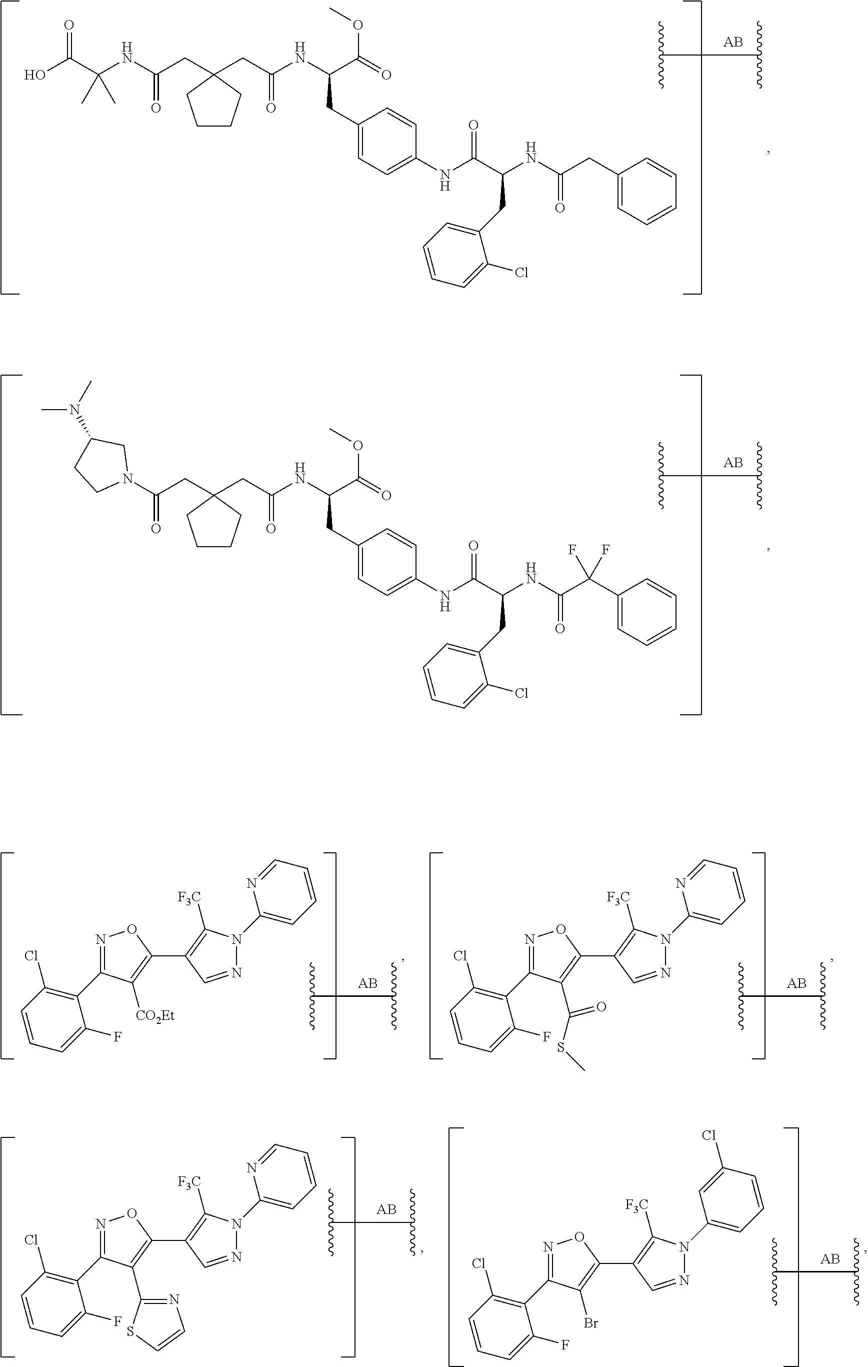

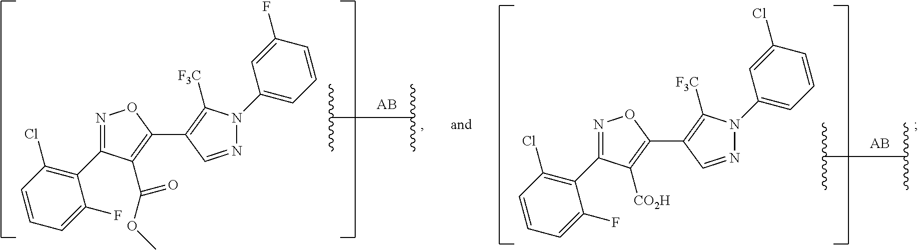

each of which is optionally substituted with 1, 2, 3, or 4 substituents independently selected from R²¹.

In certain embodiments a compound is provided of Formula

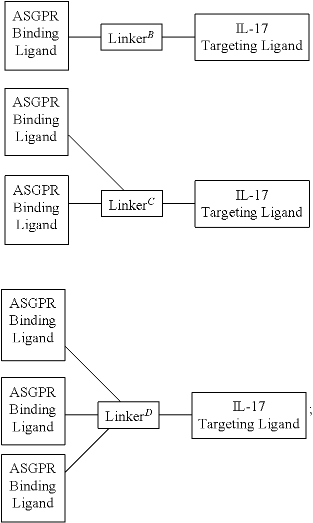

or a pharmaceutically acceptable salt thereof;
wherein
IL-17 Targeting Ligand is any IL-17 ligand described in WO2020/146,194; WO2020/163,554; WO2020/127,685; and WO2021/055,376; each of which is incorporated by reference.

In certain embodiments IL-17 Targeting Ligand is of Formula:

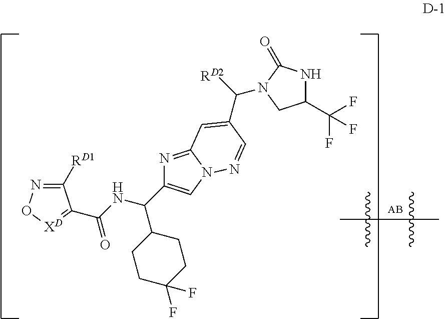

wherein,
X^{D} is CH or N;
R^{D1} is —CH₃, —CH₂F, —CHF₂, —CF₃, —CH₂CH₃, —CH₂CF₃, —CH(CH₃)₂, CH₂CHF₂, CH₂CH₂F, —CF(CH₃)₂, CF₂CH₃, —OCH₃,

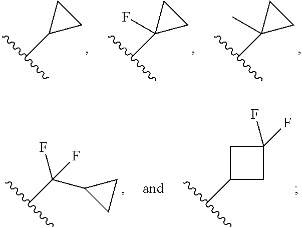

R^{D2} is —H or —CH₂OCH₃.

In certain embodiments IL-17 Targeting Ligand is of Formula:

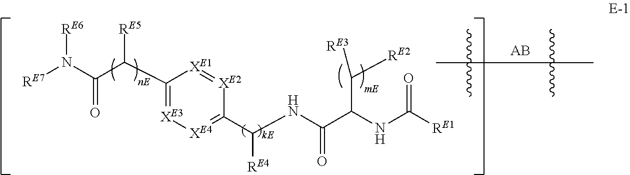

wherein,
R^{E1} is alkyl, substituted alkyl, aryl, substituted aryl, heteroaryl, substituted heteroaryl, arylalkyl, substituted arylalkyl, heteroarylalkyl, substituted heteroarylalkyl —OR^{E8} or —NR^{E9}R^{E10} or an F pocket substituent;
R^{E2} is alkyl, substituted alkyl, heterocycle, substituted heterocycle, aryl, substituted aryl, fused cycloalkylaryl, substituted fused cycloalkylaryl, heteroaryl, substituted heteroaryl or a D pocket substituent;
each R^{E3} is independently hydrogen, (C₁-C₇) alkyl, (C₁-C₇) substituted alkyl or —OR^{E32};
m^{E} is 0, 1 or 2;
each R^{E4} is independently hydrogen, (C₁-C₇) alkyl, (C₁-C₇) substituted alkyl, cycloalkyl substituted cycloalkyl, heterocycle or substituted heterocycle;
k^{E} is 0 or 1;
X^{E1}, X^{E2}, X^{E3} and X^{E4} are independently —N— or —CR^{E11}— provided that no more than two of X^{E1}, X^{E2}, X^{E3} and X^{E4} are nitrogen;
each R^{E5} is independently hydrogen, (C₁-C₇) alkyl, (C₁-C₇) substituted alkyl, heterocycle, substituted heterocycle, cycloalkyl, substituted cycloalkyl, heterocyclealkyl, substituted heterocyclealkyl, —NR^{E12}R^{E13}—NR^{E14}C(O)R^{E15}, —NHSO₂R^{E31}, OH or a B pocket substituent;
R^{E6} is hydrogen or alkyl;
R^{E7} is heterocycle, substituted heterocycle, —(CHR^{E16})ₒR^{E17} or —(CHR^{E18})ₚR^{E19} or R^{E6} and R^{E7} taken together with the nitrogen atom to which they are attached form piperazine, substituted piperazine, heterocycle or substituted heterocycle,

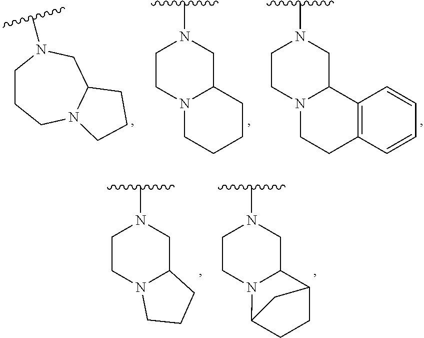

or an A pocket substituent;
R^{E8} is (C₁-C₇) alkyl, (C₁-C₇) substituted alkyl, aryl, substituted aryl, heteroaryl or substituted heteroaryl;
each R^{E11} is independently hydrogen, alkyl, substituted alkyl, —OR^{E20}, —NR^{E21}R^{E22}, halo, —CN, —CO₂R^{E23}, —CONR^{E24}R^{E25}, or —SR^{E26};
n^{E} is 1, 2 or 3;
o^{E} is 1, 2 or 3;
p^{E} is 1, 2 or 3;
each R^{E16} is independently hydrogen, (C₁-C₇) alkyl or (C₁-C₇) substituted ALKYL
R^{E17} is

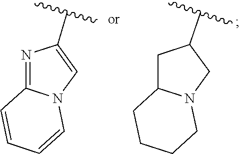 [SMILES: *c1cn2ccccc2n1 ; *C1CC2CCCCN2C1]

each R^{E18} is independently hydrogen, (C₁-C₇) alkyl, or (C₁-C₇) substituted alkyl;
R^{E19} is —NR^{E27}R^{E28};
R^{E27} and R^{E28} together with the nitrogen atom to which they are attached form a heterocycle or substituted heterocycle ring or

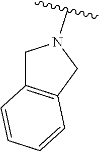 [SMILES: *N1Cc2ccccc2C1]

R^{E9}, R^{E10}, R^{E12}, R^{E13}, R^{E14}, R^{E15}, R^{E18}, R^{E19}, R^{E20}, R^{E21}, R^{E22}, R^{E23}, R^{E24}, R^{E25}, R^{E26}, R^{E30}, R^{E31}, and R^{E32} are independently selected at each instance from hydrogen, alkyl, substituted alkyl, heterocycle, substituted heterocycle, aryl, substituted aryl, heteroaryl, substituted heteroaryl, or alternatively, independently, R^{E9} and R^{E10}, R^{E21} and R^{E22} and R^{E24} and R^{E25} together with atom to which they are attached form a cycloalkyl, substituted cycloalkyl, cycloheteroalkyl or substituted cycloheteroalkyl ring;
R^{E28} is hydrogen or alkyl;
A-pocket substituent is selected from the group consisting of

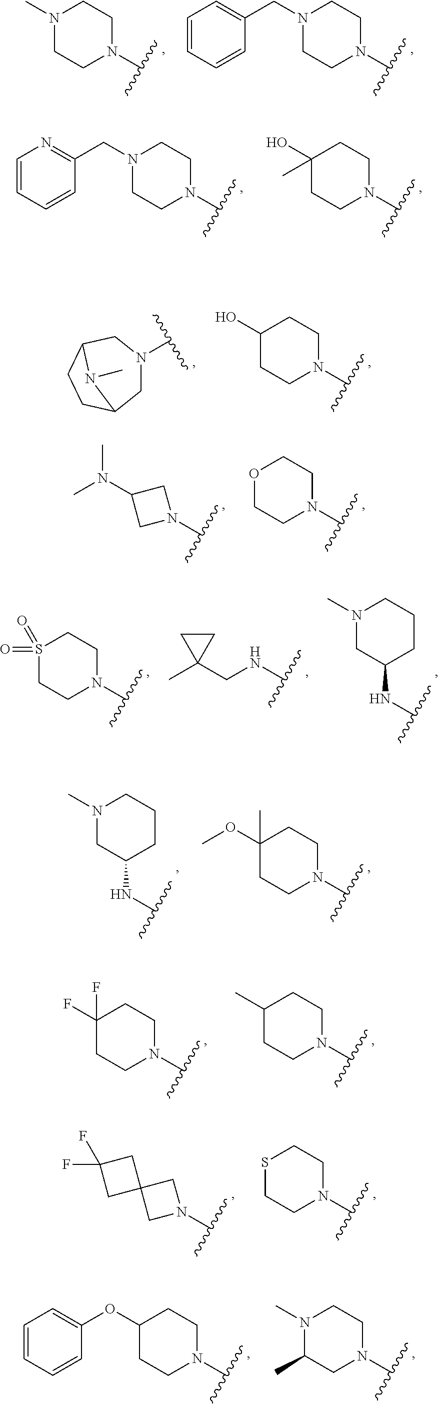

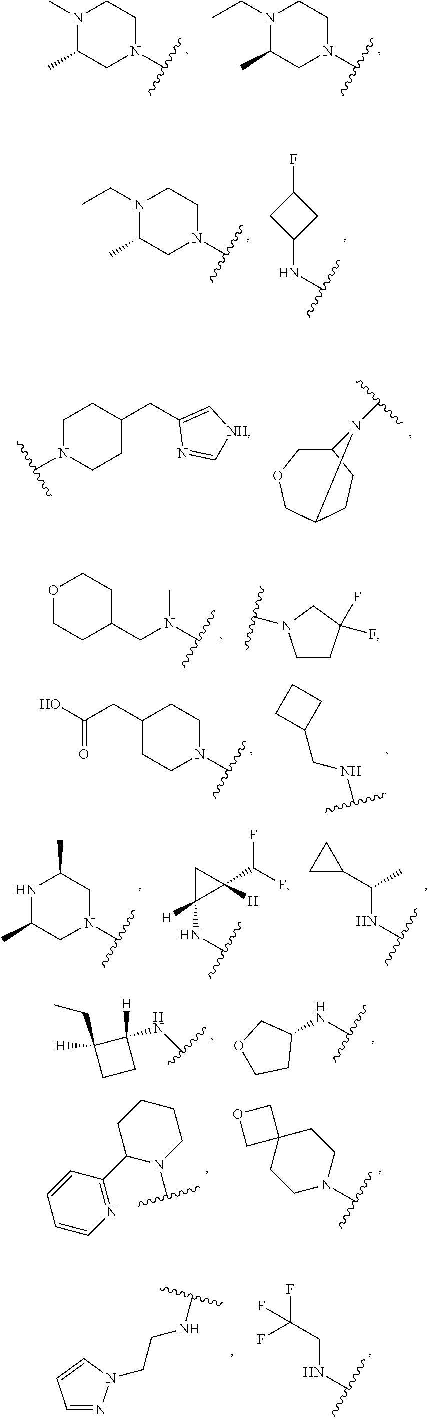

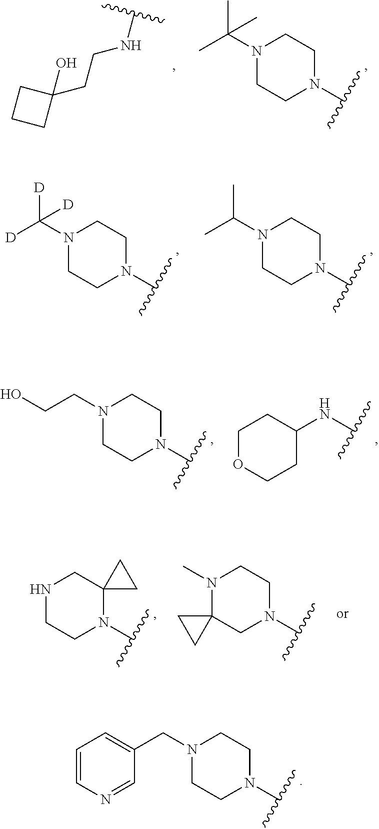

B-pocket substituent is selected from the group consisting of

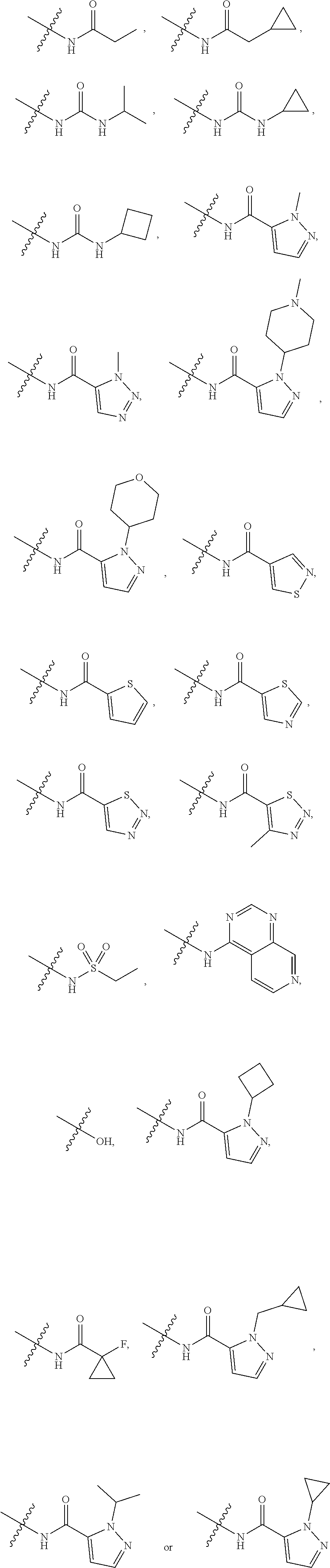

D-pocket substituent is selected from the group consisting of

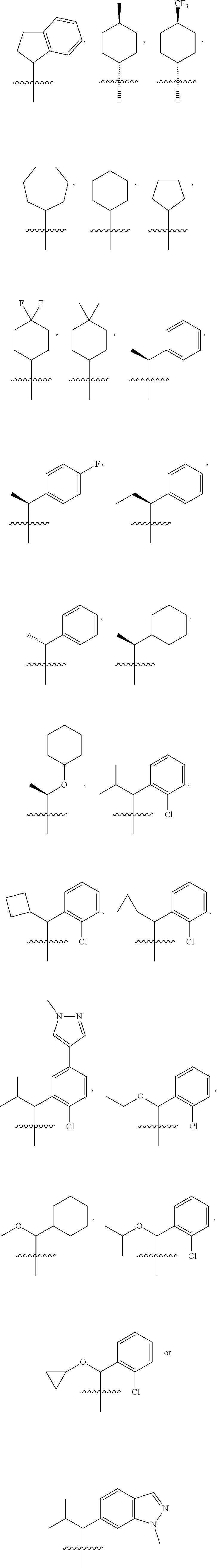

F-pocket substituent is selected from the group consisting of

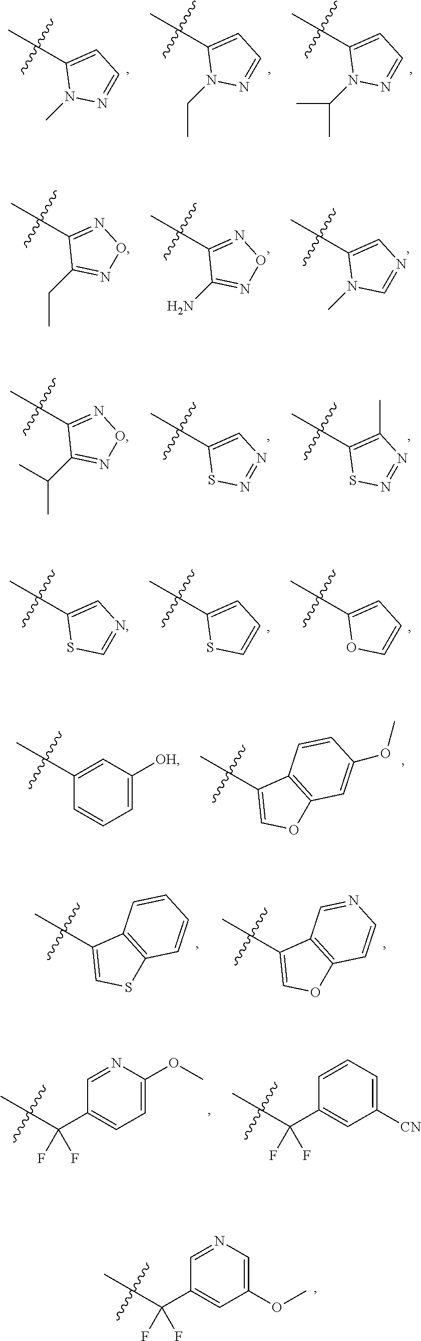

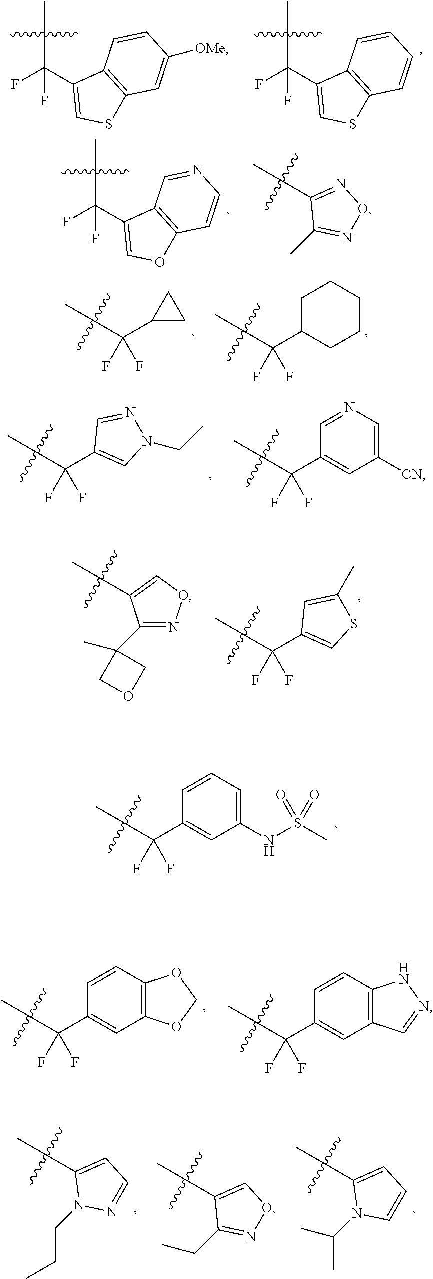

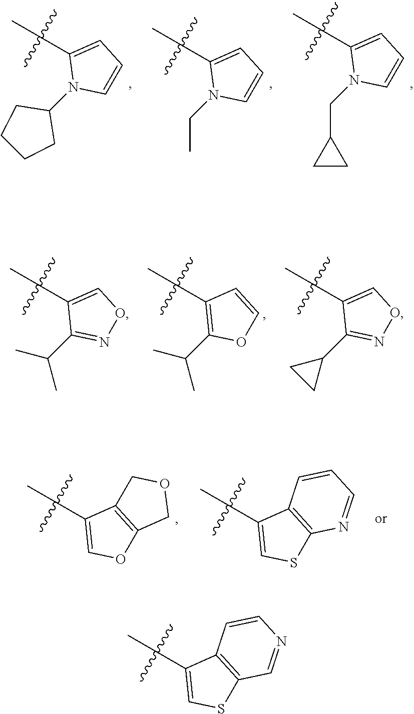

wherein each optional substituent for the above Formula is independently selected from halogen, —OR^{F12}, —SR^{F12}—N(R^{F12})₂, —C(O)R^{F12}, —C(O)N(R^{F12})₂, N(R^{F12})C(O)R^{F12}, —C(O)OR^{F12}, —OC(O)R^{F12}, —S(O)R^{F12}, —S(O)₂R^{F12}, —NO₂, ═O, ═S, ═N(R^{F12}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F12}, —N(R^{F12})₂, —C(O)R^{F12}, —C(O)N(R^{F12})₂, —N(R^{F12})C(O)R^{F12}, —C(O)OR^{F12}—OC(O)R^{F12}, —NO, ═O, ═N(R^{F11}) and —CN.

In certain embodiments the I-17 Targeting Ligand is of Formula:

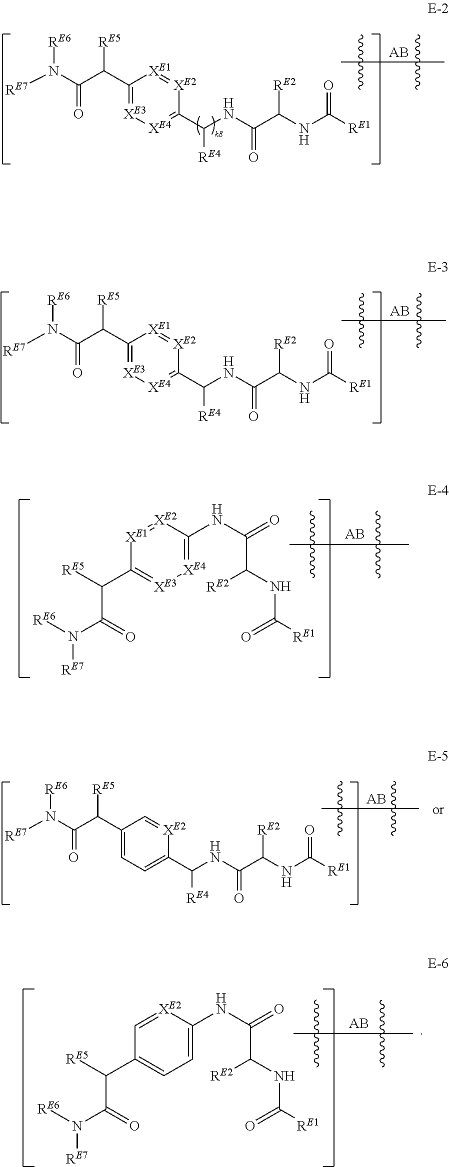

In certain embodiments IL-17 Targeting Ligand is of Formula:

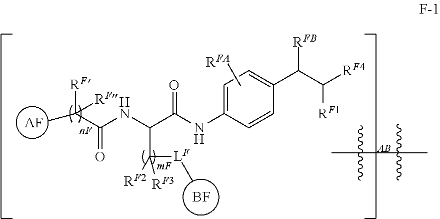

wherein,

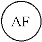

is selected from an optionally substituted C₃₋₁₂ carbocycle and optionally substituted 3- to 12-membered heterocycle wherein substituents on Ring AF are independently selected at each occurrence from:
halogen, —OR^{F11}, —SR^{F11}, —N(R^{F11})₂, —C(O)R^{F11}, —C(O)N(R^{F11})₂, N(R^{F11})C(O)R^{F11}, —N(R^{F11})S(O)₂R^{F11}, —C(O)OR^{F11}, OC(O)R^{F11}, S(O)R^{F11}, S(O)₂R^{F11}, —NO₂, ═O, ═S, ═N(R^{F11}), —CN; and
C₁₋₁₀ alkyl, C₂₋₁₀ alkenyl, C₂₋₁₀ alkynyl, each of which is optionally substituted with one or more substituents independently selected from halogen, —OR^{F11}, —SR^{F11}, —N(R^{F11})₂, —C(O)R^{F11}, —C(O)N(R^{F11})₂, —N(R^{F11})C(O)R^{F11}, —C(O)OR^{F11}, OC(O)R^{F11}, S(O)R^{F11}, S(O)₂R^{F11}, —NO₂, ═O, ═S, ═N(R^{F11}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F11}, —N(R^{F11})₂, —C(O)R^{F11}, —C(O)N(R^{F11})₂, —N(R^{F11})C(O)R^{F11}, —C(O)OR¹¹, —OC(O)R¹¹, —NO₂, ═O═N(R¹¹ and —CN·′ and
C₃₋₁₂ carbocycle and 3- to 12-membered heterocycle, each of which is optionally substituted with one or more substituents independently selected from: halogen, —OR^{F11}, SR^{F11}, —N(R^{F11})₂, —C(O)R^{F11}, —C(O)N(R^{F11})₂, N(R^{F11})C(O)R^{F11}, —C(O)OR^{F11}, —OC(O)R^{F11}, —NO₂, —CN, C₁₋₆ alkyl and C₁₋₆ haloalkyl;

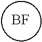

is selected from an optionally substituted C₃₋₁₀ carbocycle and optionally substituted 3- to 12-membered heterocycle each substituent on Ring B are independently selected at each occurrence from:
halogen, —OR^{F12}, —SR^{F12}, —N(R^{F12})₂, —C(O)R^{F12}, —C(O)N(R^{F12})₂, —N(R^{F12})C(O)R^{F12}, —C(O)OR^{F12}, —OC(O)R^{F12}, —S(O)R^{F12}, —S(O)₂R^{F12}, —NO₂, ═O, ═S, ═N(R^{F12}), —CN; and
C₁₋₁₀ alkyl, C₂₋₁₀ alkenyl, C₂₋₁₀ alkynyl, each of which is optionally substituted with one or more substituents independently selected from halogen, —OR^{F12}, —SR^{F12}, —N(R^{F12})₂, —C(O)R^{F12}, —C(O)N(R^{F12})₂, N(R^{F12})C(O)R^{F12}, —C(O)OR^{F12}—OC(O)R^{F12}, —S(O)R^{F12}, —S(O)₂R^{F12}—NO₂, ═O, ═S, ═N(R^{F12}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F12}, —N(R^{F12})₂, —C(O)R^{F12}, —C(O)N(R^{F12})₂, —N(R^{F12})C(O)R^{F12}, —C(O)OR^{F12}, —OC(O)R^{F12}, —NO, ═O, ═N(R^{F11}) and —CN;
R^{F4} is selected from —C(O)N(R^{F23})(R^{F24}) and C(O)heterocycle, wherein heterocycle is optionally substituted with 1, 2, 3, or 4 substituents selected from halogen, —OR^{F13}, SR^{F13}, —N(R^{F13})₂, —C(O)R^{F13}, —C(O)N(R^{F13})₂, —N(R^{F13})C(O)R^{F13}, —C(O)OR^{F13}, —OC(O)R^{F13}, —S(O)R^{F13}, —S(O)₂R^{F13}, —NO₂, ═O, ═S, ═N(R^{F13})—CN; and
C₁₋₁₀ alkyl, C₂₋₁₀ alkenyl, C₂₋₁₀ alkynyl, each of which is optionally substituted with one or more substituents independently selected from halogen, —OR^{F13}, —SR^{F13}, —N(R^{F13})₂, —C(O)R^{F13}, —C(O)N(R^{F13})₂, N(R^{F13})C(O)R^{F13}, —C(O)OR^{F13}, —OC(O)R^{F13}, —S(O)R^{F13}, —S(O)₂R^{F13}, —NO₂, ═O, ═S, ═N(R^{F13}), —CN, C₃₋₁₀ carbocycle and 3- to 10-membered heterocycle, wherein the C₃₋₁₀ carbocycle and 3- to 10-membered heterocycle are each optionally substituted with one or more substituents selected from: halogen, —OR^{F13}, —N(R^{F13})₂, —C(O)R^{F13}, —C(O)N(R^{F13})₂, —N(R^{F13})C(O)R^{F13}, —C(O)OR^{F13}, —OC(O)R^{F13}, —NO₂, ═O, ═N(R^{F13}), and —CN;
L^{F} is bond or selected from —O— and —NH—;
R^{FA} is selected from hydrogen, halogen, —OR^{F14}, —N(R^{F14})₂, —C(O)R^{F14}, —C(O)N(R^{F14})₂, N(R^{F14})C(O)R^{F14}, —C(O)OR^{F14}, —OC(O)R^{F14}, —NO₂, —CN, and C₁₋₆ alkyl, wherein C₁₋₆ alkyl is optionally substituted with one or more substituents selected from: halogen, OR^{F14}, —N(R^{F14})₂, —C(O)R^{F14}, NO₂, ═O, and —CN;
R^{FB} is selected from hydrogen, halogen, —OR^{F15}, —N(R^{F15})₂, —C(O)R^{F15}, —C(O)N(R^{F15})₂, N(R^{F15})C(O)R^{F15}, —C(O)OR^{F15}, —OC(O)R^{F15}, —NO2, —CN, and C₁₋₆ alkyl, wherein C₁₋₆ alkyl is optionally substituted with one or more substituents selected from: halogen, OR^{F15}, —N(R^{F15})₂, —C(O)R^{F15}, NO₂, ═O, and —CN, wherein at least one of R^{A} or R^{B} is not hydrogen;
R^{F}′ and R^{F}″ are independently selected from: hydrogen, halogen, —OR^{F16}, and C₁₋₆ alkyl; wherein the C₁₋₆ alkyl is optionally substituted with one or more substituents selected from: halogen, —OR^{F16}, —N(R^{F16})₂, —C(O)R^{F16}, —NO₂, ═O, and —CN;
R^{F1} is selected from —OR^{F21}, —N(R^{F21})(R^{F22}), —N(R^{F21})C(O)R^{F22}, —N(R^{F21})C(O)OR^{F22}, —N(R^{F21})C(O)N(R^{F21})(R^{F22}), —N(R^{F21})S(═O)₂N(R^{F21})(R^{F22}) and —N(R^{F21})S(═O)₂(R^{F22});
each R^{F2} and R^{F3} are independently selected from: hydrogen, halogen, —OR^{F17}, C₁₋₆ alkyl, and C₃₋₆ cycloalkyl; wherein the C₁₋₆ alkyl and C₃₋₆ cycloalkyl are optionally substituted with one or more substituents selected from: halogen, —OR^{F17}, —N(R^{F17})₂, C(O)R^{F17}, —NO₂, ═O, and —CN; or
R^{F2} and R^{F3} bound to the same carbon come together to form a C₃₋₆ cycloalkyl optionally substituted with one or more substituents selected from halogen, —OR^{F17}, —N(R^{F17})₂, C(O)R^{F17}, —NO₂, ═O, and —CN;
R^{F21} is independently selected at each occurrence from hydrogen and C₁-C₆ alkyl optionally substituted by one or more substituents independently selected from halogen, —OR^{F17}, —N(R^{F17})₂, —C(O)R^{F17}, —NO₂, ═O, and —CN;
R^{F22} is selected from: C₁₋₁₀ alkyl, C₂₋₁₀ alkenyl, C₂₋₁₀ alkynyl, each of which is optionally substituted with one or more substituents independently selected from halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, ═O, ═S, ═N(R^{F18}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, N(R^{F18})C(O)R^{F18}, —C(O)OR^{F18}, —OC(O)R^{F18}, —NO₂, ═O, ═N(R^{F18}), and —CN; and C₃₋₁₂ carbocycle and 3- to 12-membered heterocycle, each of which is optionally substituted with one or more substituents independently selected from:
halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}—NO₂, ═O, ═S, ═N(R^{F18}), —CN; and C₁₋₁₀ alkyl, C₂₋₁₀ alkenyl, C₂₋₁₀ alkynyl, each of which is optionally substituted with one or more substituents independently selected from halogen, —OR^{F18}, —SR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —S(O)R^{F18}, —S(O)₂R^{F18}, —NO₂, ═O, ═S, ═N(R^{F18}), —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, —N(R^{F18})C(O)R^{F18}, —C(O)OR^{F18}, —OC(O)R^{F18}, —NO═O, ═N(R^{F18}), and —CN; and
C₃₋₁₀ carbocycle and 3- to 10-membered heterocycle are each optionally substituted with one or more substituents selected from: halogen, —OR^{F18}, —N(R^{F18})₂, —C(O)R^{F18}, —C(O)N(R^{F18})₂, N(R^{F18})C(O)R^{F18}, —C(O)OR^{F18}, —OC(O)R^{F18}, —NO₂, ═O, ═N(R^{F18}), and —CN;
R^{F23} is selected from:
C₁₋₆ alkyl optionally substituted with one or more substituents independently selected from halogen, —OR^{F19}, —SR^{F19}—N(R^{F19})₂, —NO₂, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OR^{F19}, —N(R^{F19})₂, ═O, C₁-C₆ alkyl, C₁-C₆ haloalkyl, and —CN; and
C₃₋₁₂ carbocycle and 3- to 10-membered heterocycle, each of which is optionally substituted with one or more substituents independently selected from halogen, —OR^{F19}, —N(R^{F19})₂, ═O, C₁-C₆ alkyl, C₁-C₆ haloalkyl, and —CN;
R^{F24} is selected from hydrogen and C₁₋₆ alkyl optionally substituted with one or more substituents independently selected from halogen, —OR^{F19}, SR^{F19}, —N(R^{F19})₂, —NO₂, —CN, C₃₋₆ carbocycle and 3- to 6-membered heterocycle;
R^{F11}, R^{F12}, R^{F13}, R^{F14}, R^{F15}, R^{F16}, R^{F17}, R^{F18}, and R^{F19} are independently selected at each occurrence from
hydrogen; and
C₁₋₆ alkyl optionally substituted with one or more substituents independently selected from halogen, —OH, —O—C₁-C₆ alkyl, —O—C₁-C₆haloalkyl-NH2, —NO2, ═O, —CN, C₃₋₁₀ carbocycle and 3- to 10-membered heterocycle; wherein the C₃₋₁₀ carbocycle and 3- to 10-membered heterocycle are each optionally substituted with one or more substituents selected from: halogen, —OH, —O—C₁-C₆ alkyl, —O—C₁-C₆ haloalkyl —NH₂, —NO₂, ═O, and —CN; and
C₃₋₁₂ carbocycle and 3- to 12-membered heterocycle, each of which is optionally substituted with one or more substituents independently selected from:
halogen, —OH, —O—C₁-C₆ alkyl, —O—C₁-C₆haloalkyl —NH₂, —NO₂, ═O, —CN; and
C₁₋₆ alkyl optionally substituted with one or more substituents independently selected from halogen, —OH, —O—C₁-C₆ alkyl, —O—C₁-C₆ haloalkyl —NH₂, —NO₂, ═O, and —CN;
n^{F} is selected from 0 and 1; and
m^{F} is selected from 0, 1, and 2.

In certain embodiments IL-17 Targeting Ligand is of Formula:

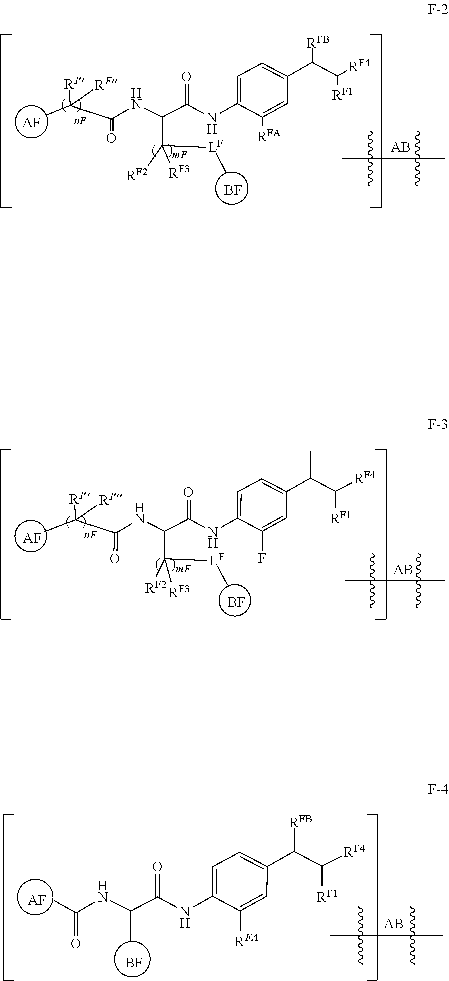

In certain embodiments IL-17 Targeting Ligand is of Formula:

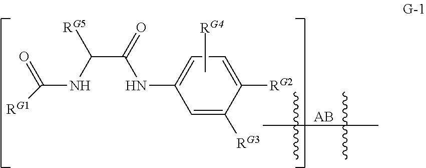

wherein,
RG1 is selected from the group consisting of 5- or 6-membered heteroaryl, 9- or 10-membered bicyclic heteroaryl, phenyl, (C₁-C₆)alkoxy, (C₃-C₇)cycloalkoxy, (C₁-C₆)alkyl, phenyl-(C₁-C₄)alkyl, (C3-C₇)cycloalkyl, 4-6-membered heterocycloalkyl and —NR^{GC}R^{GD}, wherein said 5- or 6-membered heteroaryl, 9- or 10-membered bicyclic heteroaryl, phenyl, (C₁-C₆)alkoxy, (C₃-C₇)cycloalkoxy, (C₁-C₆)alkyl, phenyl-(C₁-C₄)alkyl, (C₃-C₇)cycloalkyl and 4-6-membered heterocycloalkyl is optionally substituted with one or more substituents independently selected from R^{GA};
R^{GA} represents deuterium, halogen, hydroxy, —NR^{GC}R^{GD}, (C₁-C₆)alkyl, (C₁-C₆)alkylcarbonyl, (C₃-C₇)cycloalkyl, phenyl, 5- or 6-membered heteroaryl or, 4-6-membered heterocycloalkyl, wherein said (C₁-C₆)alkyl, (C₁-C₆)alkylcarbonyl, (C₃-C₇)cycloalkyl, phenyl, 5- or 6-membered heteroaryl or 4-6-membered heterocycloalkyl is optionally substituted with one or more substituents independently selected from deuterium, halogen, hydroxy, cyano, (C₁-C₄)alkyl, (C₃-C₇)cycloalkyl, (C₁-C₄)alkoxy, —SO₂—(C₁-C₄)alkyl and —NR^{GC}R^{GD};
R^{G2} is selected from the group consisting of 5- or 6-membered heteroaryl, wherein said 5- or 6-membered heteroaryl is optionally substituted with one or more substituents independently selected from R^{GB}, wherein said 5- or 6-membered heteroaryl may optionally contain —CO— as a ring member and wherein when said 5 membered heteroaryl contains nitrogen as a ring atom said nitrogen may optionally be substituted with a substituent selected from R^{G8};
R^{GB} represents deuterium, halogen, cyano, hydroxy, —NR^{GC}R^{GD}, (C₁-C₆)alkyl, (C₁-C₆)alkoxy, (C₁-C₆)alkyl-CO—O—(CH₂)ₙ— or (C3-C₇)cycloalkyl, wherein n is 1-4, and wherein said (C₁-C₆)alkyl, (C₁-C₆)alkoxy or (C₃-C₇)cycloalkyl is optionally substituted with one or more substituents independently selected from deuterium, halogen, cyano, hydroxy, —NR^{GC}R^{GD} and (C₁-C₄)alkoxy;
R^{GC} and R^{GD} each independently are selected from the group consisting of hydrogen and (C₁-C₆)alkyl, or R^{GC} and R^{GD} together form pyrrolidinyl or piperidinyl, wherein said (C₁-C₆)alkyl, pyrrolidinyl or piperidinyl is optionally substituted with one or more substituents independently selected from halogen, cyano and hydroxy;
R^{G8} is selected from the group consisting of -L^{G}-PO(OH)₂ and —CHR^{GG}O—(CO-A-NR^{GH}))_{0 or 1)}—CO-A-NR^{GH}R^{GI};
L^{G} is selected from the group consisting of a bond or —CHR^{GG}O—;
wherein each —CO-A-NR^{GH}— independently represents an amino acid residue wherein the amino acid residue is selected from the natural amino acids either in D or L-form or as mixtures of the D and L form, and wherein said amino acid residue may be substituted on the a-amino group with a substituent R^{GH};
R^{GG}, R^{GH}, and R^{GI} are independently selected from hydrogen and (C₁-C₆) alkyl;
R^{G3} is selected from the group consisting of hydrogen, deuterium, hydroxy and halogen;
R^{G4} is selected from the group consisting of hydrogen, deuterium and halogen;
R^{G5} is selected from the group consisting of —CHR^{G6}R^{G7}, (C₃-C₁₀)cycloalkyl and GG, wherein said (C₃-C₁₀)cycloalkyl and GG are optionally substituted with one or more substituents independently selected from deuterium, halogen, cyano, hydroxy, (C₁-C₄)alkyl and halo(C₁-C₄)alkyl;
GG represents

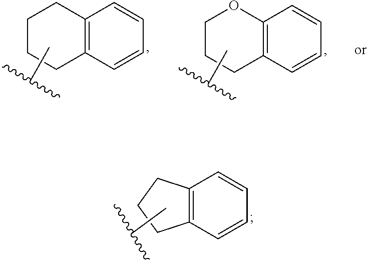

and
R^{G6} and R^{G7} each independently represents hydrogen, phenyl, (C₁-C₆)alkyl, or (C₃-C₇)cycloalkyl, wherein said phenyl, (C₁-C₆)alkyl or (C₃-C₇)cycloalkyl is optionally substituted with one or more substituents independently selected from halogen, cyano, hydroxy and (C₁-C₄)alkyl.

In certain embodiments IL-17 Targeting Ligand is of Formula:

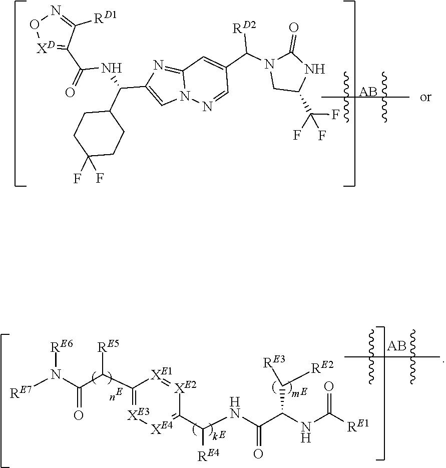

### Interleukin-6 (IL-6)

In some embodiments, the Target Extracellular Protein is human interleukin-6 (IL-6) (UniProtKB—P05231 (IL6_HUMAN)). IL-6 is a cytokine with a wide variety of biological functions. It is a potent inducer of the acute phase response and plays an essential role in the final differentiation of B-cells into Ig-secreting cells. It is also involved in lymphocyte and monocyte differentiation. It also acts on B-cells, T-cells, hepatocytes, hematopoietic progenitor cells and cells of the CNS, and is required for the generation of T(H)17 cells. IL-6 has been implicated in a number of inflammatory diseases and cancers, including, but not limited to, Castleman's disease, metastatic castration-associated prostate cancer, renal cell carcinoma, large-cell lung carcinoma, ovarian cancer, rheumatoid arthritis, asthma.

The Protein Data Bank website provides the crystal structure of IL-6 searchable by 1P9M (Boulanger, M. J., et al., Science, 2003, 300: 2101-2104); 1ALU (Somers et al., EMBO J., 1997, 16, 989-997); 1IL6 and 2IL6 (Xu, G. Y., et al., J Mol Biol., 1997, 268 468-481) and 1N26 (Varghese et al., Proc Natl Acad Sci USA., 2002, 99 15959-15964); as well as the crystal structure of IL-6 bound to various compounds searchable by 4CNI (Shaw, S., et al., Mabs, 2014, 6: 773); and 4NI7 and 4NI9 (Gelinas et al., J Biol Chem. 2014, 289(12), 8720-8734). Additionally, Gelinas et al., provides insight into the crystal structure of interleukin-6 in complex with a modified nucleic acid ligand (Gelinas, A. D., et al., J Biol Chem. 2014, 289(12), 8720-8734); and Somers et al., provides insight into the crystal structure of interleukin 6: implications for a novel mode of receptor dimerization and signaling.

Non-limiting examples of IL-6 direct or indirect inhibitors are provided in FIG. 1. Additional IL-6 direct or indirect inhibitors can be found in, for example, U.S. Pat. No. 8,901,310; U.S. patent Ser. No. 10/189,796; U.S. Pat. No. 9,694,015; each incorporated herein by reference. In another embodiment the IL-6 Extracellular Targeting Ligand is AvimarC326 or a binding fragment thereof which is described in Nat Biotechnol 23, 1556-1561 (2005).

In some embodiments, the Target Extracellular Protein is Interleukin-6. Interleukin-6 (IL-6) is a cytokine that is a crucial component of the acute phase immune response. IL-6 ligand binds to IL-6 receptor, and the heterodimer then associates with IL6ST and gp130, stimulating a response. During infection certain molecules from pathogens bind to toll-like receptors, which activate macrophages to produce IL-6. In addition to stimulating differentiation of B cells and neutrophils, IL-6 mediates the fever response.

IL-6 has been implicated in many inflammatory diseases, including multiple sclerodid, neuromyelitis optica spectrum disorder, diabetes, atherosclerosis, depression, Alzheimer's disease, systemic lupus erythromatosus, multiple myeloma, prostate cancer, Behcet's disease, rheumatoid arthritis, systemic juvenile idiopathic arthritis, and Castleman's disease.

IL-6 signaling is also important to the musculoskeletal system. In bone, it interacts with VEGF stimulating angiogenesis. In muscle cells, IL-6 is produced in large amounts during exercise. In contrast to its role in stimulating the immune system, during exercise IL-6 is anti-inflammatory.

The Protein Data Bank website provides the crystal structure of Interleukin-6, searchable by 1ALU (Somers, W. S. et al. 1.9 A crystal structure of interleukin 6: implications for a novel mode of receptor dimerization and signaling. (1997) EMBO J. 16: 989-997), 1IL6 (Xu, G. Y. et al. Solution structure of recombinant human interleukin-6 (1997) J Mol Biol 268: 468-481), and the structure of IL-6 bound in the active hexameric complex searchable by 1P9M (Boulanger, M. J. et al. Hexameric Structure and Assembly of the Interleukin-6/IL-6-alpha-Receptor/gp130 Complex. (2003) Science 300: 2101-2104)

Non-limiting examples of IL-6 Targeting Ligands can be found in, for example, USP 10633423, USP 10669314, US 2004/0092720, and Ranganath, S. et al. Discovery and Characterization of a Potent Interleukin-6 Binding Peptide with Neutralizing Activity In Vivo.

PLOS ONE 10(11): e0141330.
SEQ ID NO: 350
QSDChaDCIHRLLEAF(4-F)LDPNLTEEQRWEKIGlaKINDECE
(Ranganath, S. et al. PLOS ONE 10(11): e0141330)
SEQ ID NO: 351
QSDChaDCIHRLLEAF(4-F)
LDPNLTEEQRWERIGlaK(PEG30L)INDECE
(Ranganath, S. et al. PLOS ONE 10(11): e0141330)
SEQ ID NO: 352
QSDChaDCIHRLLEAF(4-F)
LDPNLTEEQRWERIGlaK(PEG20Br)INDECE
(Ranganath, S. et al. PLOS ONE 10(11): e0141330)
SEQ ID NO: 353
QSDChaDCIHRLLEAF(4-F)
LDPNLTEEQRWERIGlaK(PEG40Br)INDECE
(Ranganath, S. et al. PLOS ONE 10(11): e0141330)
SEQ ID NO: 354
FDhLDCIHRLLEAFLDPNLTEQQRWEKIDKINDECE
(Ranganath, S. et al. PLOS ONE 10(11): e0141330)
SEQ ID NO: 355
QSDChaDCIHRLLEAF(4-F)LDPNLTEEQRWERIGlaKINDECE
(Ranganath, S. et al. PLOS ONE 10(11): e0141330)
SEQ ID NO: 356
SWQSDChaDCIHRLLEAFLDK-AcNLTEEQRWERIDKINDECE
(Ranganath, S. et al. PLOS ONE 10(11): e0141330)
SEQ ID NO: 357
SWQSDChaDCIHRLLEAFLDK-PEG40BrNLTEEQRWERIDKINDECE
(Ranganath, S. et al. PLOS ONE 10(11): e0141330)

In certain embodiments the IL-6 Targeting ligand is SEQ ID NO:343, bound to the linker through the PEGylated lysine residue.

SEQ ID NO: 358
EEX₃X₄AWX-EIHX₁₁LPNLX₁₆X₁₇X₁₈QX₂₀X₂₁AFIX₂₅X₂₆LX₂₈X₂₉
(U.S. Pat. No. 10,633,423)

wherein, independently from each other,
X₃ is selected from A, F, H, K, Q, R, S, W and Y;
X₄ is selected from A, D, E, F, H, I, K, L, M, N, Q, R, S, T, V and Y;
X₇ is selected from F, H, I, K, L, M, N, R, S, T, V, W and Y;
X₁₁ is selected from A, I, K, L, M, N, R, S, T and V;
X₁₆ is selected from N and T;
X₁₇ is selected from A, I, T and V;
X₁₈ is selected from D, E, G, H, K, N, Q, R, S and T;
X₂₀ is selected from I, L, M, R, T and V;
X₂₁ is selected from A, S, T and V;
X₂₅ is selected from I, M, Q, S, T, V and W;
X₂₆ is selected from K and S,
X₂₈ is selected from F, L, M and Y; and
X₂₉ is selected from D and R;

SEQ ID NO: 359
EEX₃X₄AWX-EIHX₁₁LPNLX₁₆X₁₇X₁₈QX₂₀X₂₁AFIX₂₅X₂₆LX₂₈X₂₉
(U.S. Pat. No. 10,669,314)

wherein, independently from each other,
X₃ is selected from A, F, H, K, Q, R, S, W and Y;
X₄ is selected from A, D, E, F, H, I, K, L, M, N, Q, R, S, T, V and Y;
X₇ is selected from F, H, I, K, L, M, N, R, S, T, V, W and Y;
X₁₁ is selected from A, I, K, L, M, N, R, S, T and V;
X₁₆ is selected from N and T;
X₁₇ is selected from A, I, T and V;
X₁₈ is selected from D, E, G, H, K, N, Q, R, S and T;
X₂₀ is selected from I, L, M, R, T and V;
X₂₁ is selected from A, S, T and V;
X₂₅ is selected from I, M, Q, S, T, V and W;
X₂₆ is selected from K and S;
X₂₈ is selected from F, L, M and Y; and
X₂₉ is selected from D and R;

In certain embodiments the targeting ligand for treating an IL-6 mediated disease binds to gp130. Non-limiting examples of gp130 Targeting Ligands can be found in, for example, Ahn, S-H. et al. In vitro and in vivo pharmacokinetic characterization of LMT-28 as a novel small molecular interleukin-6 inhibitor 2020 Asian-Australas J Anim Sci. 33:670-677, Aqel, S. I. Novel small molecule IL-6 inhibitor suppresses autoreactive Th17 development and promotes Treg development. (2019) Clinical and Experimental Immunology, 196:215-225, Hong, S.-S. et al. A Novel Small-Molecule Inhibitor Targeting the IL-6 Receptor beta Subunit, Glycoprotein 130. 2015 J Immunol 195:237-245

In certain embodiments the gp130 binding Targeting Ligand is selected from

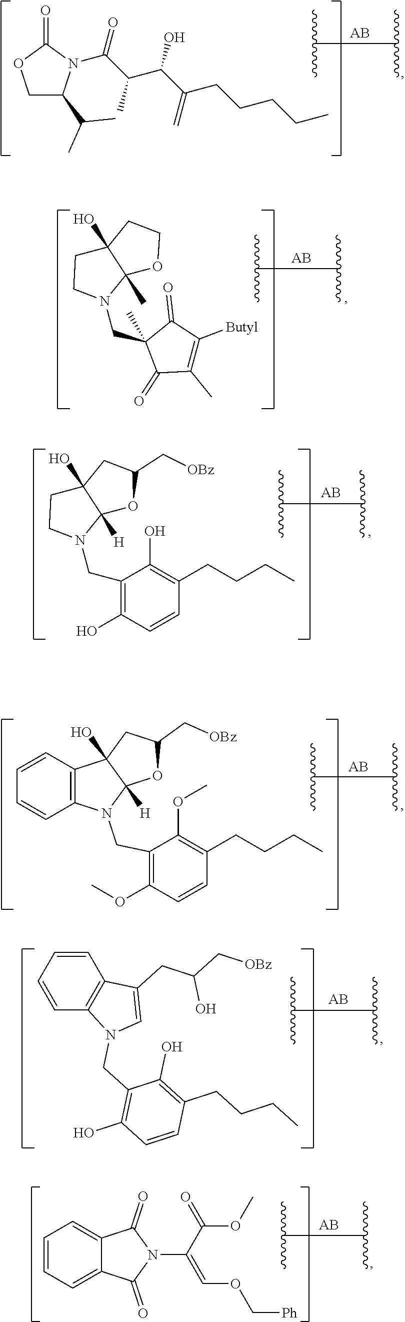

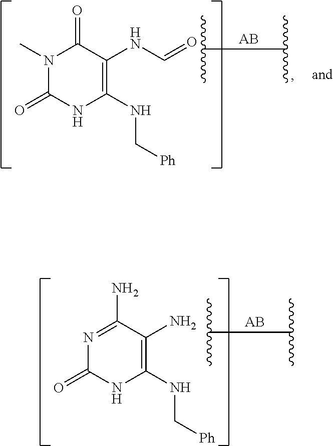

### Immunoglobulin A1 (IgA1)

Immunoglobulin A is a class of antibodies which is commonly found in secretions, but is also present in serum. IgA contains four heavy chains and four light chains, in a dimeric form. IgA exists in two isotypes, IgA1 and IgA2. IgA1 contains more repeats in the hinge region and is the predominant form found in serum. While production of IgA maintains strong mucosal immunity and defending against pathogens, it can become toxic. IgA nephropathy, also known as Berger's disease, is the pathological buildup of IgA antibodies which reduces kidney function. The etiology of the disease remains unclear, however it has been suggested that the glycosylation pattern on the hinge region plays a role. As proper kidney function is important for overall health, IgA nephropathy is associated with systemic diseases such as liver failure, cancer, celiac disease, systemic lupus erythematosus, rheumatoid arthritis, heart failure, reactive arthritis, and ankylosing spondylitis.

The Protein Data Bank website provides the crystal structure of IgA1, and representative example include PDB accession codes 1IGA (Boehm, M. K. 1999, J. Mol. Bio. 286 1421-1447) 2ESG (Almogren, A. 2006 J. Mol. Biol. 356, 413-431), 6XJA, 7JGJ, (Eisenmesser, E. Z. 2020, Nat. Commun, 11, 6063-6063), and 3CHN (Bonner, A. 2009, Mucosal Immunol., 2, 74-84).

Direct or indirect IgA1 binding molecules include jacalin and

SEQ ID NO: 360
YYALSDAKEEEPRYKALRGENQDLREKERKYQDKIKKLEEKEKN
LEKKS.

Anti-β1AR Autoantibody Targeting Ligand In certain aspects a Extracellular Protein Degrading Compound of the present invention degrades anti-β1AR autoantibodies and can be used to treat a disorder mediated by anti-β1AR autoantibodies such as heart failure, for example cardiomyopathy or dilated cardiomyopathy. In certain embodiments the anti-β1AR autoantibody Targeting Ligand is SEQ. ID 361 DEARRCYNDPKCSDFVQ. In certain embodiments, the anti-β1AR autoantibody targeting ligand has about 98%, 95%, 93%, 90%, 88%, 85%, 83%, or 80% sequence homology with SEQ. ID 361.

In other embodiments the anti-β1AR autoantibody Targeting Ligand is SEQ. ID 362 5′-ggttggtgtggttgg-3′. In certain embodiments, the anti-β1AR autoantibody targeting ligand has about 93%, 86%, or 80% sequence homology with SEQ. ID 362. In other embodiments an aptamer described in Werner et al. “The aptamer BC 007 for treatment of dilated cardiomyopathy: evaluation in Doberman Pinschers of efficacy and outcomes” ESC Heart Failure, 2020, 7, 844-855 is used as an anti-β1AR autoantibody Targeting Ligand. In certain aspects an aptamer such as SEQ. ID 362 may provide lower immunogenicity than a protein-based targeting ligand.

### Proprotein Convertase Subtilisin/Kexin Type 9 (PCSK-9)

In some embodiments, the Target Extracellular Protein is human proprotein convertase subtilisin/kexin type 9 (PCSK-9) (UniProtKB—Q8NBP7 (PCSK9_HUMAN)). PCSK-9 is a crucial player in the regulation of plasma cholesterol homeostasis. PCSK-9 binds to low-density lipid receptor family members: low density lipoprotein receptor (LDLR), very low-density lipoprotein receptor (VLDLR), apolipoprotein E receptor (LRP1/APOER) and apolipoprotein receptor 2 (LRP8/APOER2), and promotes their degradation in intracellular acidic compartments. It acts via a non-proteolytic mechanism to enhance the degradation of the hepatic LDLR through a clathrin LDLRAP1/ARH-mediated pathway, and may prevent the recycling of LDLR from endosomes to the cell surface or direct it to lysosomes for degradation. PCSK-9 has been implicated in high blood cholesterol and the development of cardiovascular disease.

The Protein Data Bank website provides the crystal structure of PCSK-9 searchable by 2P4E (Cunningham, D., et al., Nat Struct Mol Biol., 2007, 14 413-419); as well as the crystal structure of PCSK-9 bound to various compounds searchable by 3BPS (Kwon, H. J., et al., Proc Natl Acad Sci USA, 2008, 105 1820-1825); 6U26, 6U2N, 6U2P, 6U36, 6U38, and 6U3X (Petrilli, W. L., et al., Cell Chem Biol., 2019, 27 32-40.e3); 50CA (Gustafsen, C., et al., Nat Commun., 2017, 8 503-503); 4NE9 (Schroeder, C. I., et al., Chem Biol., 2014, 21 284-294); 40V6 (Mitchell, T., et al., J Pharmacol Exp Ther., 2014, 350 412-424); and 4NMX (Zhang, Y., et al., J Biol Chem., 2014, 289 942-955). Additionally, Piper et al., provides insight into the crystal structure of PCSK9 (Piper, D. E., et al., Structure, 2007, 15(5), 545-52).

Representative PCSK-9 Targeting Ligands are provided in FIG. 1. In some embodiments, the PCSK-9 Targeting Ligand is the peptide TVFTSWEEYLDWV (SEQ ID NO:363) (J. Bio. Chem. 2014 January; 289(2):942-955, incorporated herein by reference). Additional PCSK-9 Targeting Ligands are provided in, for example, U.S. Pat. No. 9,227,956, J Biol Chem 289: 942-55 (2014), each of which is incorporated by reference herein.

In certain embodiments the PCSK-9 ligand is any PCSK-9 ligand described in WO2021/156792 which is incorporated by reference.

In certain embodiments a compound is provided of Formula

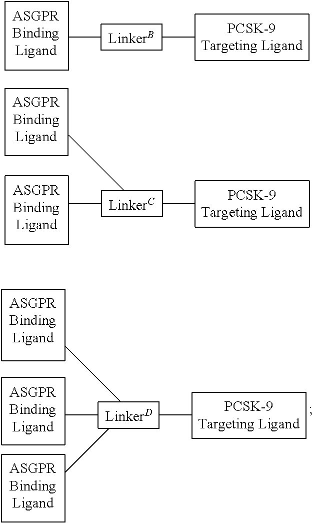

or a pharmaceutically acceptable salt thereof;
wherein
ASGPR Ligand is an ASGPR Ligand described herein;
PCSK-9 Targeting Ligand is any PCSK-9 ligand described in WO2021/156792.

Non-limiting examples of PCSK-9 Targeting Ligands that can be used in any of the formulas of the present invention include:

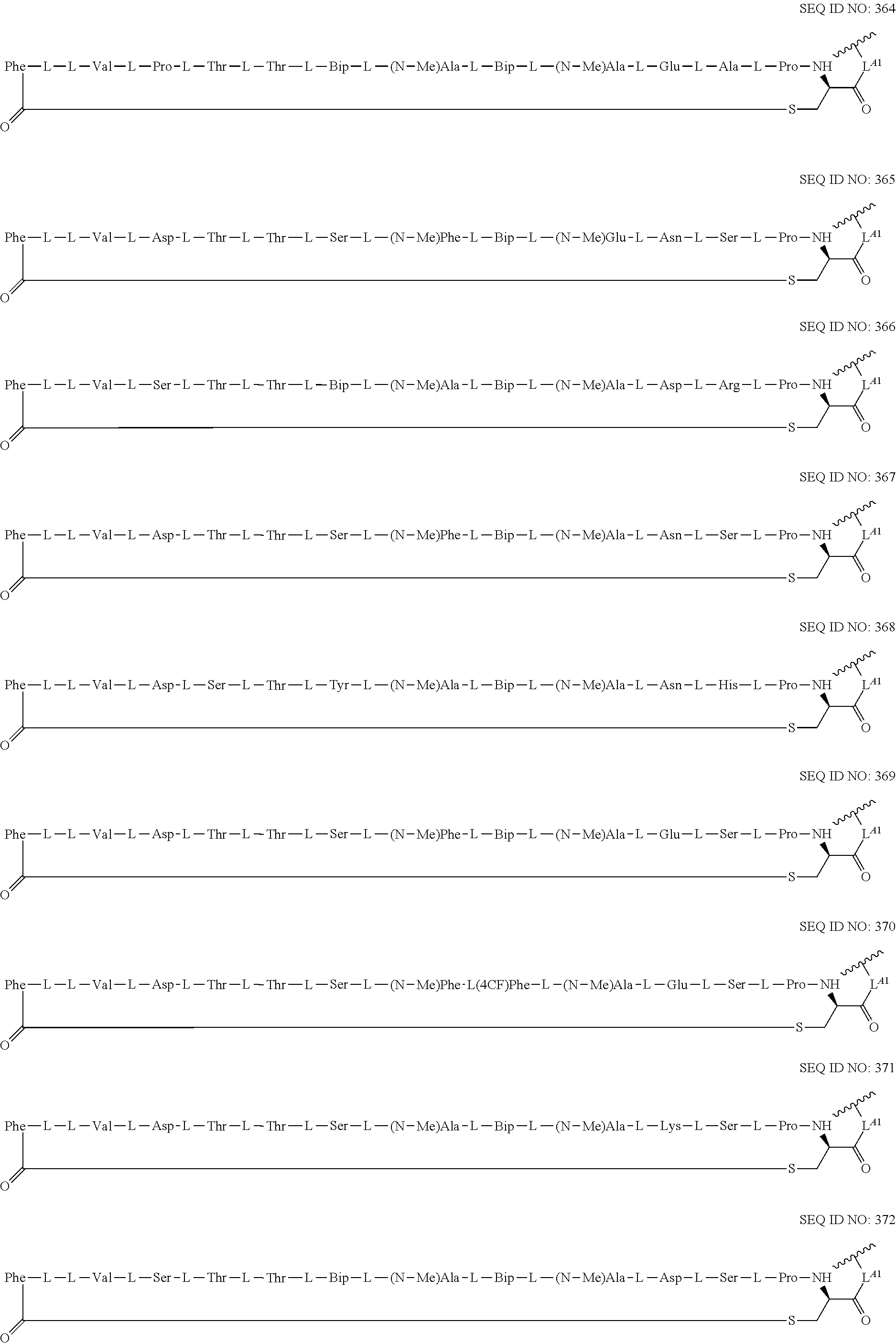

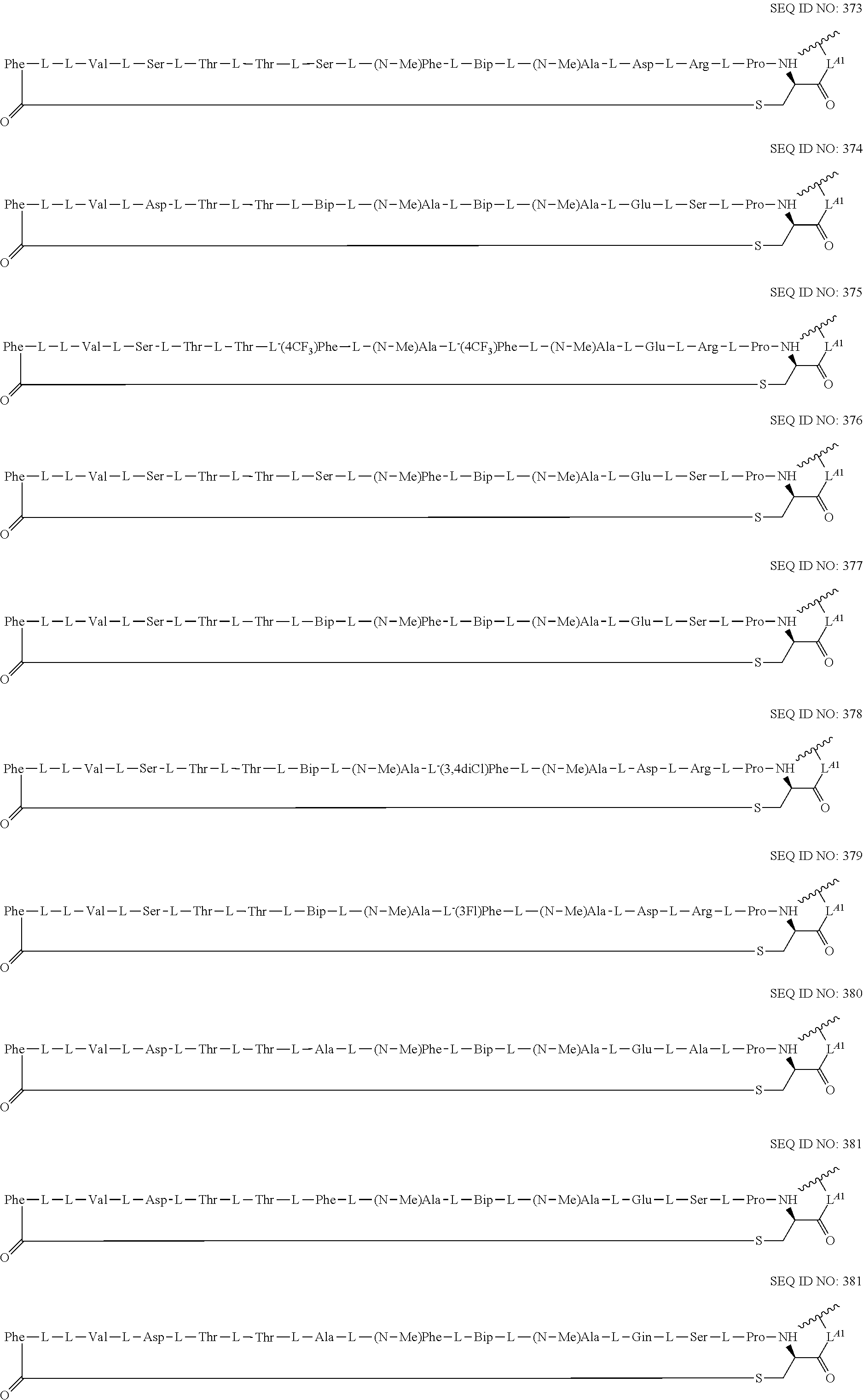

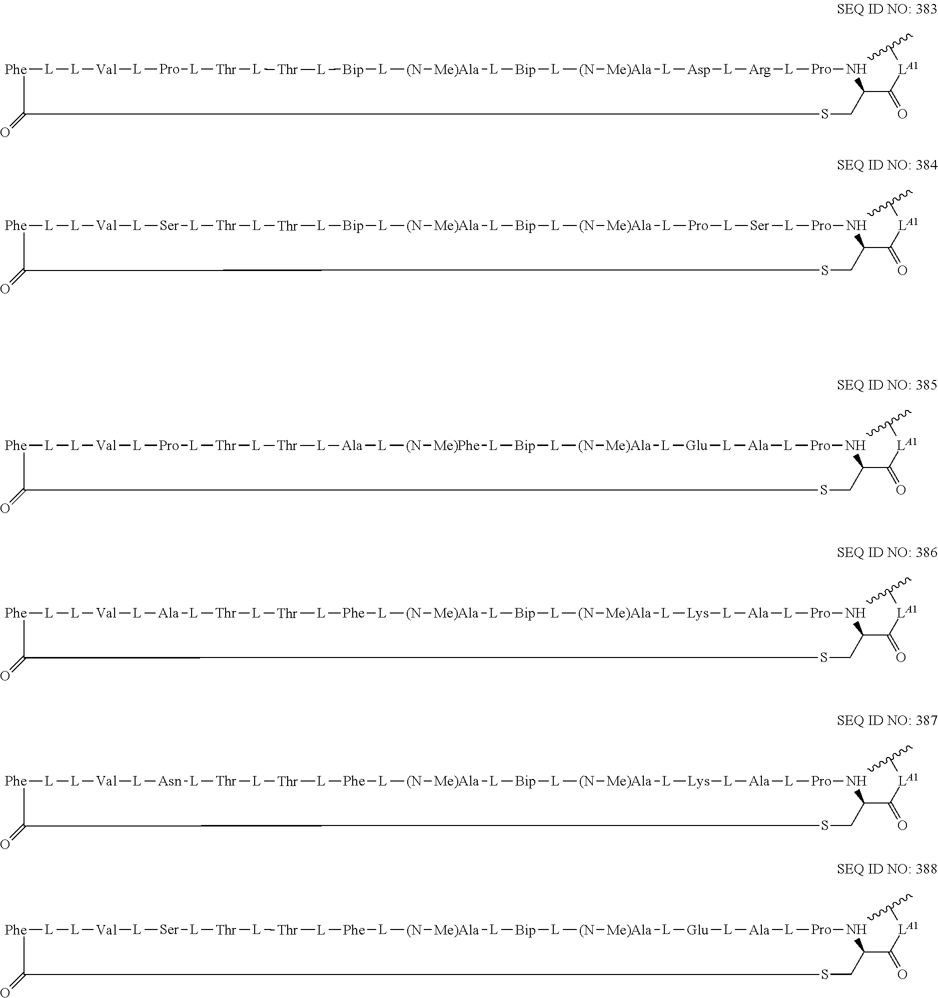

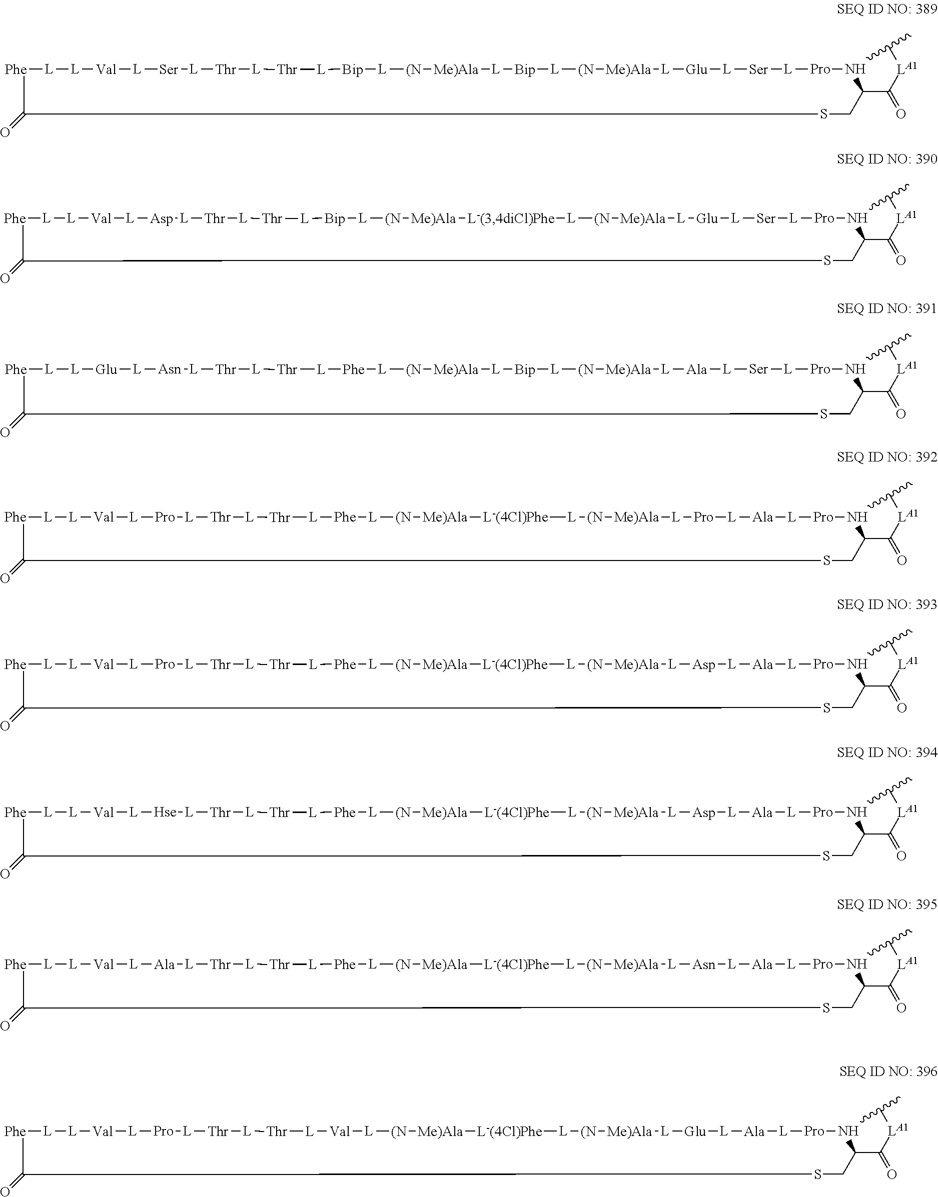

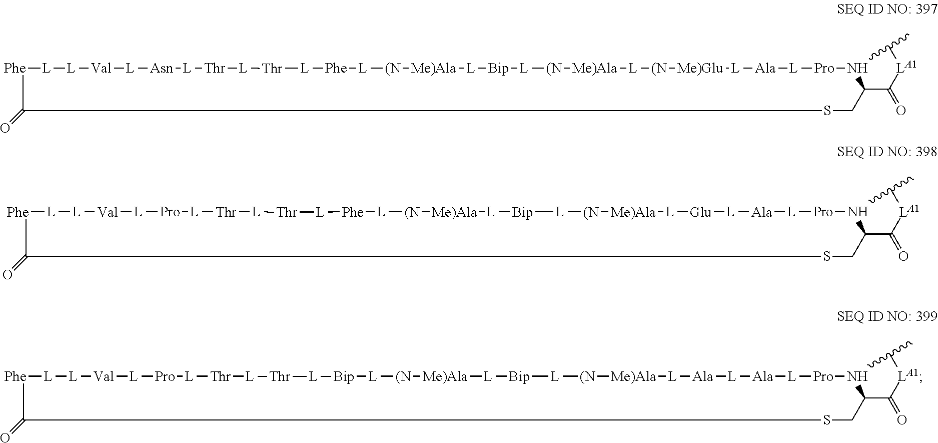

wherein L^{A1} is bond, NR⁸, or O.

In certain embodiments the PCSK9 Targeting Ligand is a compound of Formula:

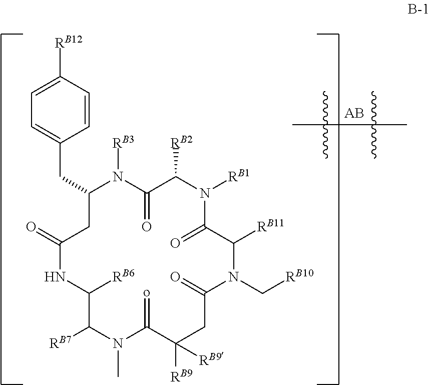

wherein,
R^{B1} is H;
R^{B2} is (C₁-C₆)alkoxy, -L^{B1}-, or (C₁-C₆)alkyl, substituted with —C(═O)OH;
R^{B3} is H or (C₁-C₆)alkyl;
R^{B6} is H, (C₁-C₆)alkyl or L^{B1};
R^{B7} is H, (C₁-C₆)alkyl or L^{B1};
or R^{B6} and R^{B7} together with the carbon atoms to which they are attached form a (C₃-C₇)cycloalkyl;
R^{B9} is H or (C₁-C₆)alkyl, optionally substituted with one or more R^{B27};
R^{B9′} is H or (C₁-C₆)alkyl;
R^{B10} is (C₆-C₁o)aryl substituted with OR^{B13} and optionally substituted with one or more R^{B14};
R¹¹ is (C₁-C₆)alkyl or L^{B1};
R^{B12} is halogen, (C₁-C₆)alkyl, (C₁-C₆)alkoxy, (C₁-C₆)haloalkyl, (C₁-C₆)haloalkoxy, —OH, or CN;
R^{B13} is (C₆-C₁o)aryl substituted with R^{B16};
each R^{B14} is independently at each occurrence halogen, (C₁-C₆)alkyl, (C₁-C₆)alkoxy, (C₁-C₆)haloalkyl, (C₁-C₆)haloalkoxy, oxo, —OH, or CN;
R^{B16} is 5- to 7-membered heteroaryl comprising 1-3 heteroatoms selected from N, O, and S, optionally substituted with one or more R^{B26};
   each R^{B26} is independently at each occurrence (C₁-C₆)alkyl optionally substituted with one or more R^{B29};
each R^{B27} is independently at each occurrence (C6-C₁₀)aryl;
each R^{B29} is independently at each occurrence —NR^{B31}R^{B32} or 4- to 7-membered heterocyclyl comprising 1-3 heteroatoms selected from N, O, and S;
each R^{B31} is independently selected from Hand (C₁-C₆)alkyl;
each R^{B32} is independently selected from Hand (C₁-C₆)alkyl; L^{B1} is —(CH₂)ₚNH—*, where the * of L^{B1} indicates the point of attachment to Linker (L_{A}), and where at least one of R^{B11}, R^{B6} or R^{B7} is -L^{B1}-; and
n is 1.

In certain embodiments the PCSK9 Targeting Ligand is a compound of Formula:

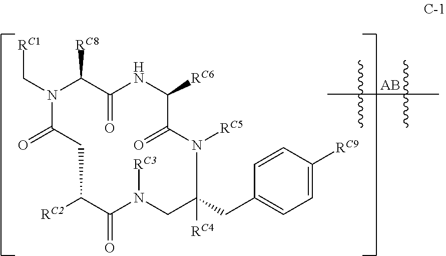

wherein,
R^{C1} is (C₆-C₁₀)aryl substituted with —OR^{C10} and one or more R^{C11};
R^{C2} is H, (C₁-C₆)alkyl, -L^{C1} or (C₃-C₉)carbocyclyl, wherein the alkyl is substituted with one R^{C18}, and the carbocyclyl is substituted with one or more R^{C19};
R^{C3} is H or (C₁-C₆)alkyl;
R^{C4} is H or (C₁-C₆)alkyl; or
R^{C3} and R^{C4} together with the atoms to which they are attached form a 5- to 7-membered heterocyclyl ring comprising 1-3 heteroatoms selected from N, O, and S;
R^{C5} is H or (C₁-C₆)alkyl;
R^{C6} is (C₁-C₆)alkyl, or -L^{C1}, wherein the alkyl is optionally substituted with one or more substituents each independently selected from —OH or (C₁-C₆)alkoxy;
R^{C8} is H, (C₁-C₆)alkyl, or -L^{C1},
R^{C9} is halogen;
R^{C10} is (C₆-C₁o)aryl substituted with one R^{C22};
each R^{C11} is independently at each occurrence halogen, (C₁-C₆)alkyl, (C₁-C₆)alkoxy, (C₁-C₆)haloalkyl, (C₁-C₆)haloalkoxy, —OH, or CN;
R^{C18} is (C₆-C₁o)aryl;
each R^{C19} is independently at each occurrence halogen, (C₁-C₆)alkyl, (C₁-C₆)alkoxy, (C₁-C₆)haloalkyl, (C₁-C₆)haloalkoxy, —OH, or CN;
R^{C22} is 5- or 6-membered heteroaryl comprising 1-3 heteroatoms selected from N, O, and S, substituted with one or more R^{C23};
each R^{C23} is independently at each occurrence (C₁-C₆)alkyl, optionally substituted with —NR^{C24}R^{C25} or a 4- to 7-membered heterocyclyl comprising 1-3 heteroatoms selected from N, O, and S;
R^{C24} is H, (C₁-C₆)alkyl;
R^{C25} is H, (C₁-C₆)alkyl,
L^{C1} is —(CH₂)ₚNH—*, where the * of L^{C1} indicates the point of attachment to Linker (L_{A}), and where at least one of R^{C2}, R^{C6} or R^{C8} is -L^{C1}; and
p is 1, 2, 3, 4, 5, or 6.

In certain embodiments the PCSK-9 Targeting Ligands that can be used in any of the formulas of the present invention include:

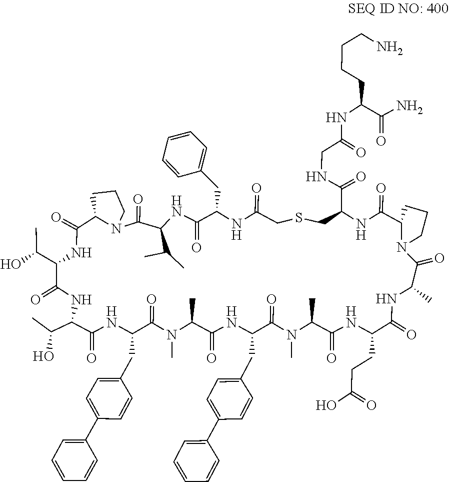 [SMILES: CC(C)[C@@H]1NC(=O)[C@H](Cc2ccccc2)NC(=O)CSC[C@@H](C(=O)NCC(=O)N[C@@H](CCCCN)C(N)=O)NC(=O)[C@@H]2CCCN2C(=O)[C@H](C)NC(=O)[C@H](CCC(=O)O)NC(=O)[C@H](C)N(C)C(=O)[C@H](Cc2ccc(-c3ccccc3)cc2)NC(=O)[C@H](C)N(C)C(=O)[C@H](Cc2ccc(-c3ccccc3)cc2)NC(=O)[C@H]([C@@H](C)O)NC(=O)[C@H]([C@@H](C)O)NC(=O)[C@@H]2CCCN2C1=O]

In certain embodiments the PCSK-9 Targeting Ligands that can be used in any of the formulas of the present invention include:

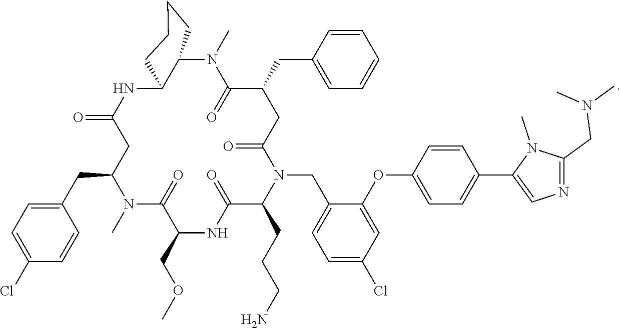

In certain embodiments the PCSK9 Targeting Ligand is selected from:

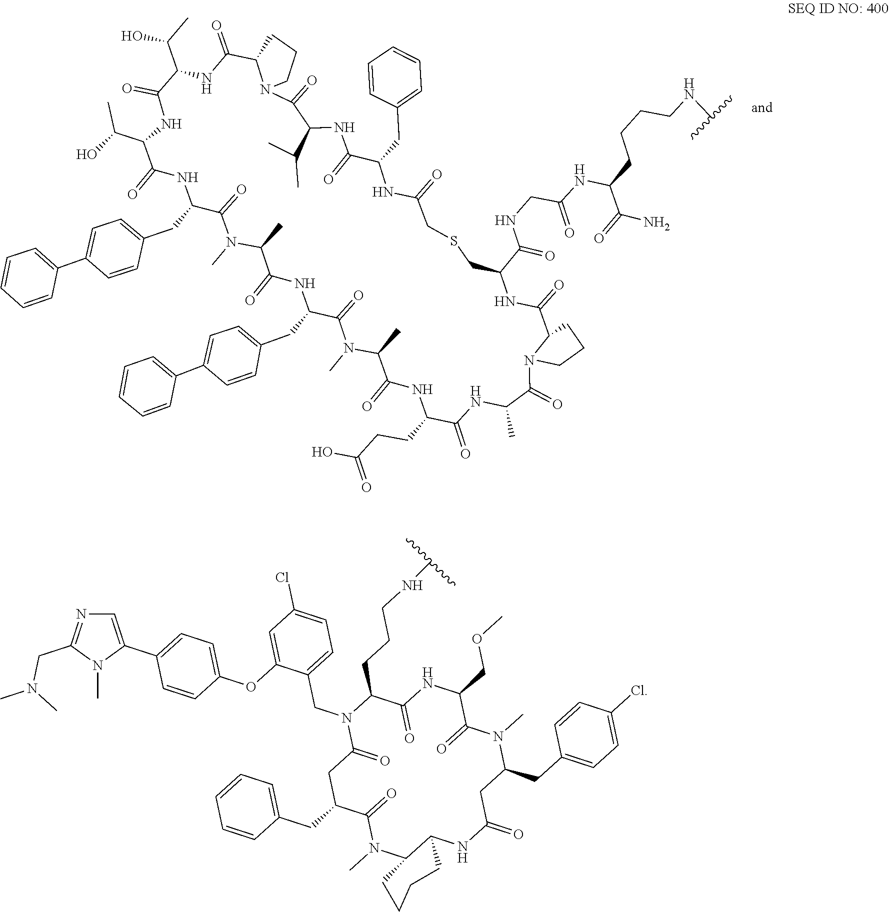

In certain embodiments, the PCSK9 ligand is a heparin mimetic, such as a PI-88 based derivative. In certain embodiments, the PCSK9 binding ligand is:

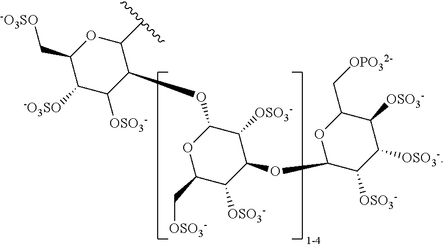

In certain embodiments, the PCSK9 ligand is

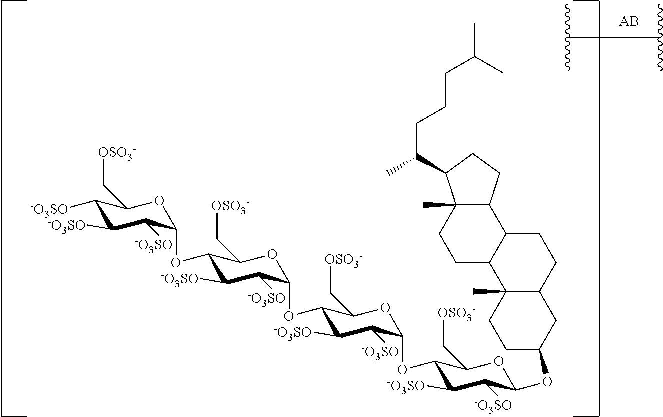

### FHR3

The human complement factor H-related protein 3 (FHR-3) belongs to the complement factor H (FH)-family. Factor H (FH), a major negative regulator of alternative complement pathway activation, belongs to a family that also includes five other related family members thought to have arisen from nonallelic homologous recombination and interlocus gene conversion including: complement factor H-related protein 1 (FHR1), complement factor H-related protein 2 (FHR2), complement factor H-related protein 3 (FHR3), complement factor H-related protein 4 with isoforms 4A and 4B (FHR4A and FHR4B) and complement factor H-related protein 5 (FHR5).

FHR3, unlike factor H, lacks the complement regulatory domains essential for complement inactivation and also competes with factor H, resulting in complement over-activation. Thus, the present invention provides compounds for use in modulating the concentration of complement factor H-proteins, specifically FHR3, to remove factor H's competitor and thereby restore factor H-mediated regulation to treat disorders caused by excessive complement activation.

Due to the central role that factor H plays in the regulation of complement, there are many clinical implications arising from aberrant FH activity. Loss of function mutation in factor H increase susceptibility to the renal diseases, atypical hemolytic uremic syndrome (aHUS) and dense deposit disease (ODD), whilst polymorphic variation of complement factor H has been strongly associated with important human diseases, including age-related macular degeneration (AMO) and meningococcal sepsis (Clin Exp Immunol 151(2):210-230; Immunobiology 217(11):1034-1046).

In certain embodiment, the invention provides the use in the treatment of a FHR3 mediated disease or disorder.

In certain embodiments, the FHR3 mediated disease or disorder is a complement-related diseases, disorders of complement dysregulation, autoimmune diseases, kidney disease, retinal degenerative diseases, Rheumatic Diseases, associated degenerative diseases, autoimmune renal disease, dense deposit disease (ODD), and systemic autoimmune diseases.

In certain embodiments, nonlimiting examples of FHR3 mediated diseases or disorders include nephropathy, age-related macular degeneration, atypical hemolytic uremic syndrome (aHUS), autoimmune form of hemolytic uremic syndrome, hepatocellular carcinoma (HCC), C3 glomerulopathy, paroxysmal nocturnal hemoglobinuria, Polymyalgia rheumatica, rheumatoid arthritis, meningococcal sepsis, and SLE (Systemic lupus erythematosus).

In certain embodiments, the present invention provides compounds that utilize receptor mediated endocytosis to eliminate or decrease level of complement factor H-related protein 3 (FHR3) from the plasma.

In certain embodiments, the FHR3 Targeting Ligand is selected from:

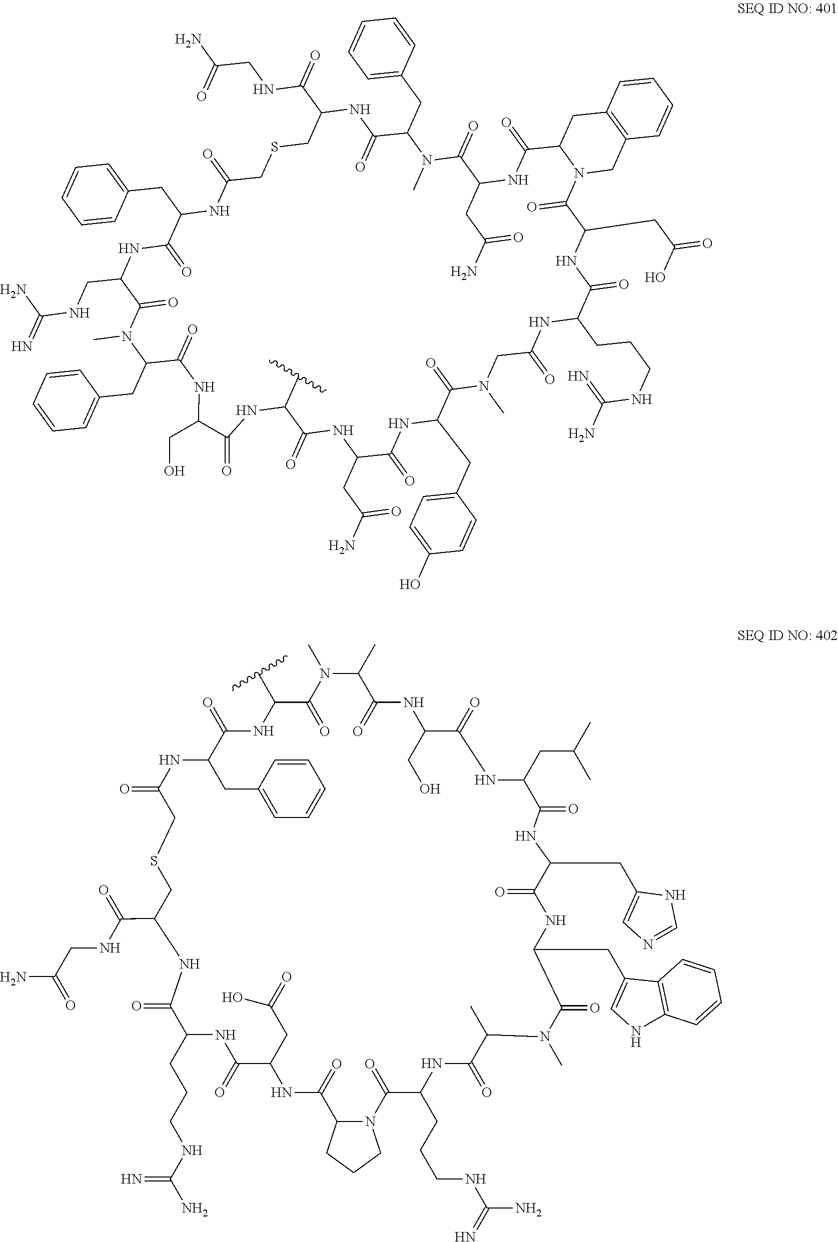 [SMILES: *C1NC(=O)C(CO)NC(=O)C(Cc2ccccc2)N(C)C(=O)C(CNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C(=O)C(CC(=O)O)NC(=O)C(CCCNC(=N)N)NC(=O)CN(C)C(=O)C(Cc2ccc(O)cc2)NC(=O)C(CC(N)=O)NC1=O ; *C1NC(=O)C(Cc2ccccc2)NC(=O)CSCC(C(=O)NCC(N)=O)NC(=O)C(CCCNC(=N)N)NC(=O)C(CC(=O)O)NC(=O)C2CCCN2C(=O)C(CCCNC(=N)N)NC(=O)C(C)N(C)C(=O)C(Cc2c[nH]c3ccccc23)NC(=O)C(Cc2cnc[nH]2)NC(=O)C(CC(C)C)NC(=O)C(CO)NC(=O)C(C)N(C)C1=O]

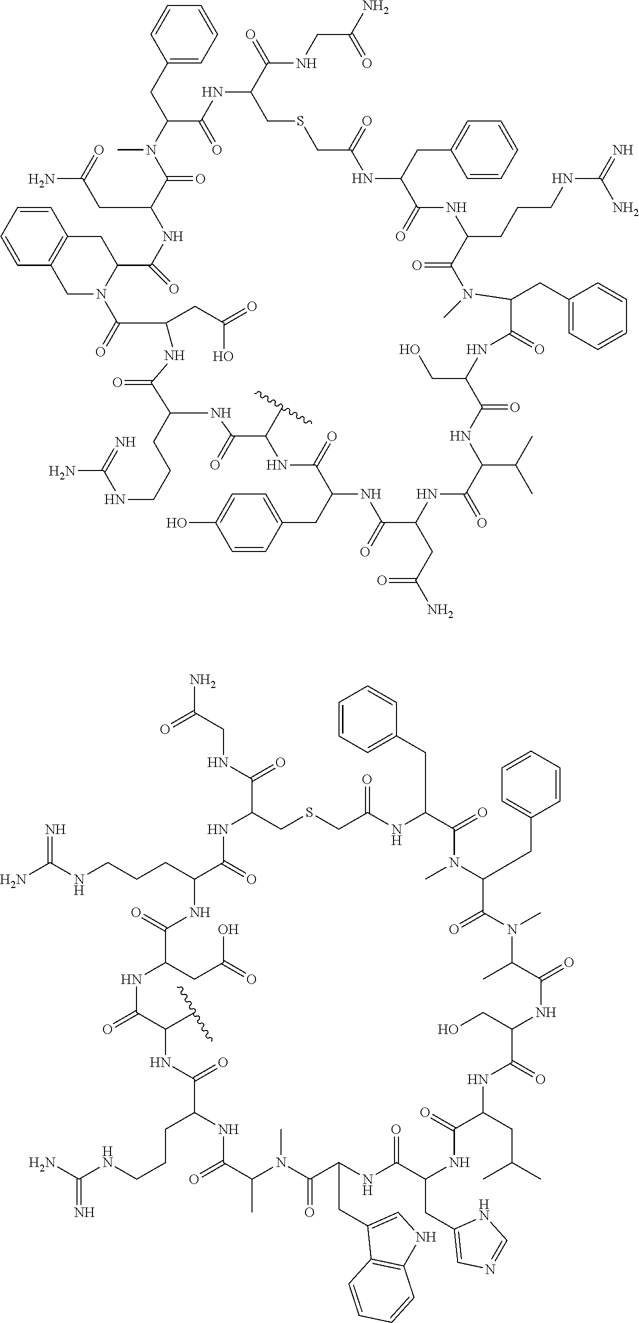 [SMILES: *C1NC(=O)C(Cc2ccc(O)cc2)NC(=O)C(CC(N)=O)NC(=O)C(C(C)C)NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C(=O)C(CC(=O)O)NC(=O)C(CCCNC(=N)N)NC1=O ; *C1NC(=O)C(CCCNC(=N)N)NC(=O)C(C)N(C)C(=O)C(Cc2c[nH]c3ccccc23)NC(=O)C(Cc2cnc[nH]2)NC(=O)C(CC(C)C)NC(=O)C(CO)NC(=O)C(C)N(C)C(=O)C(Cc2ccccc2)N(C)C(=O)C(Cc2ccccc2)NC(=O)CSCC(C(=O)NCC(N)=O)NC(=O)C(CCCNC(=N)N)NC(=O)C(CC(=O)O)NC1=O]

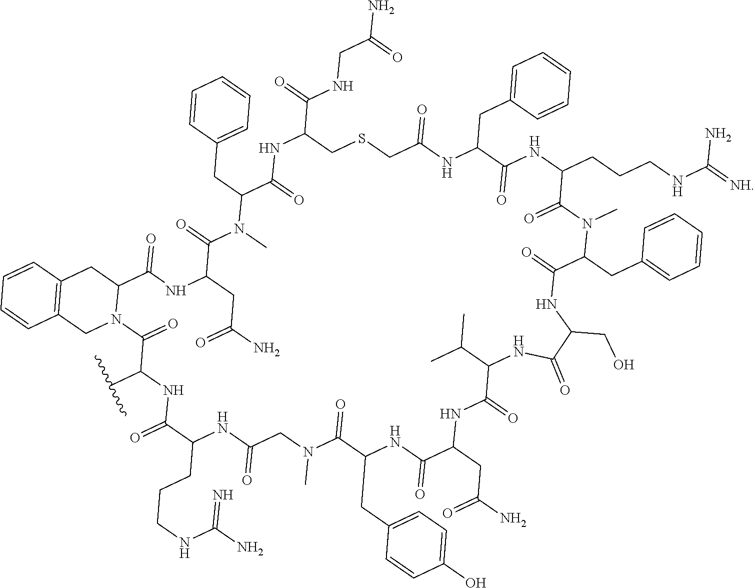 [SMILES: *C1NC(=O)C(CCCNC(=N)N)NC(=O)CN(C)C(=O)C(Cc2ccc(O)cc2)NC(=O)C(CC(N)=O)NC(=O)C(C(C)C)NC(=O)C(CO)NC(=O)C(Cc2ccccc2)N(C)C(=O)C(CCCNC(=N)N)NC(=O)C(Cc2ccccc2)NC(=O)CSCC(C(=O)NCC(N)=O)NC(=O)C(Cc2ccccc2)N(C)C(=O)C(CC(N)=O)NC(=O)C2Cc3ccccc3CN2C1=O]

In certain embodiments, the FHR3 compound is selected from:

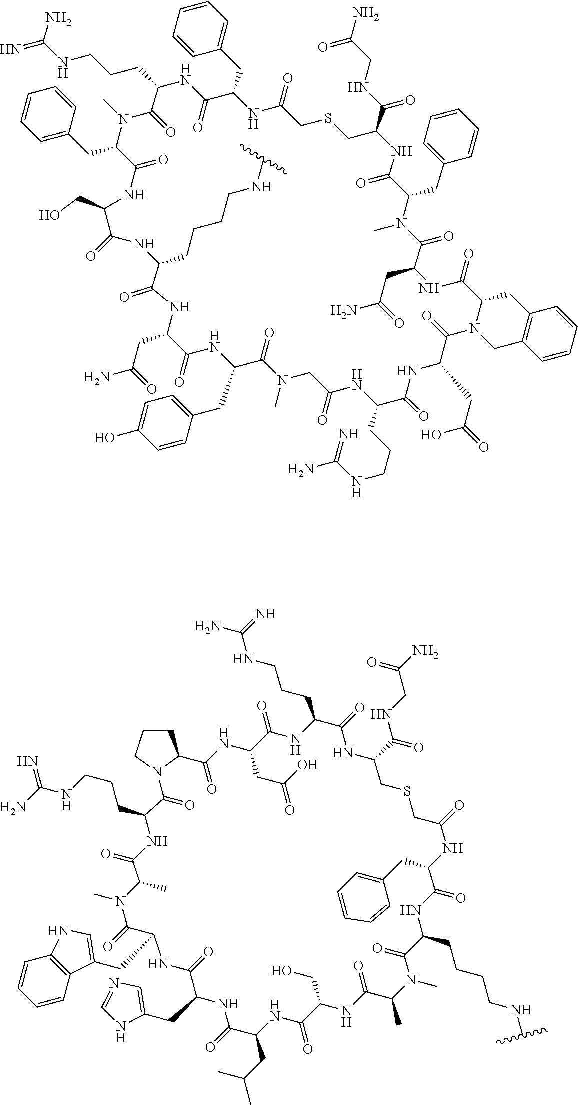

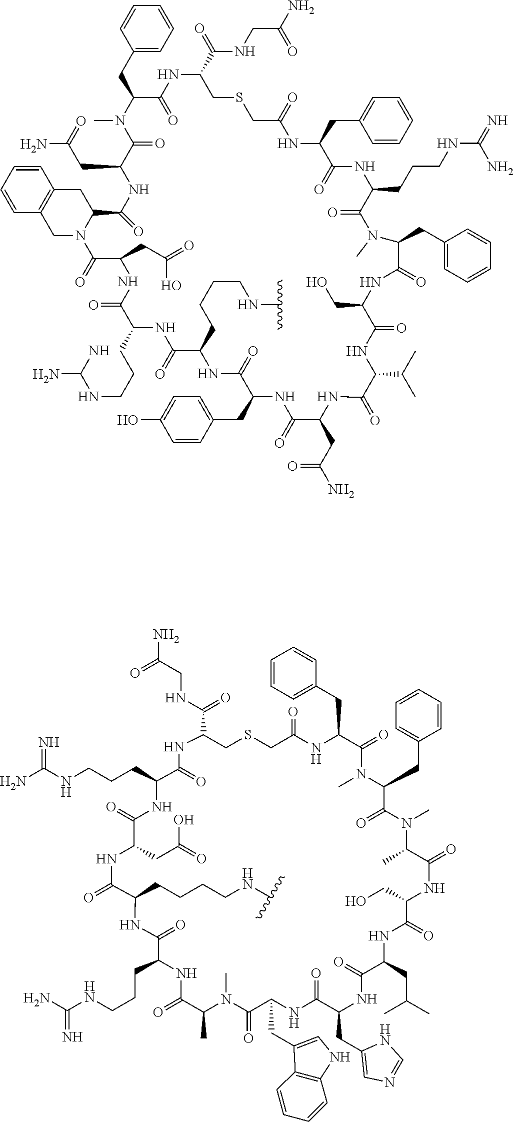

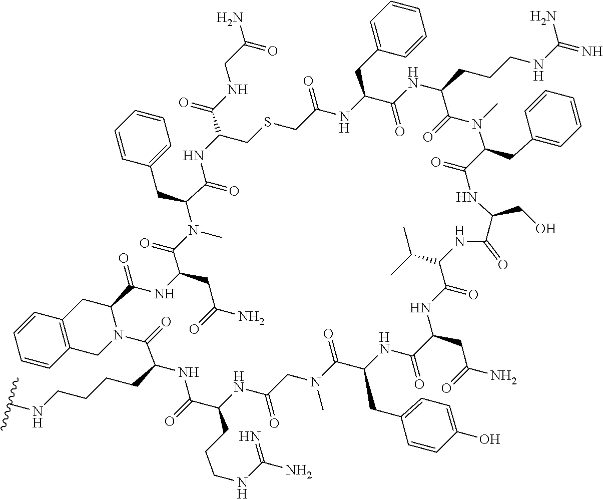

### Vascular Epithelial Growth Factor (VEGF)

In some embodiments, the Target Extracellular Protein is human vascular epithelial growth factor (VEGF) (UniProtKB—P15692 (VEGFA_HUMAN)). VEGF is a growth factor active in angiogenesis, vasculogenesis, and endothelial cell growth. VEGF induces endothelial cell proliferation, promotes cell migration, inhibits apoptosis and induces permeabilization of blood vessels. VEGF has been implicated in the vascularization and angiogenesis of tumors.

The Protein Data Bank website provides the crystal structure of VEGF searchable by 3QTK (Mandal, K., et al., Angew Chem Int Ed Engl., 2011, 50 8029-8033); and 4KZN (Shen et al.); as well as the crystal structure of VEGF bound to various compounds searchable by 504E (Lobner, E., et al., MAbs, 2017, 9 1088-1104); 4QAF (Giese, T., et al.,); 5DN2 (Tsai, Y. C. I., et al., FEBS, 2017, J 283 1921-1934); 4GLS (Mandal, K., et al., Proc Natl Acad Sci USA, 2012, 109 14779-14784); and 1KMX (Stauffer, M. E. et al., J Biomol NMR, 2002, 23 57-61). Additionally, Mueller, Y. A., et al, provides insight into the Crystal structure and functional mapping of the kinase domain receptor binding site of VEGF (Mueller, Y. A., et al., Proc Natl Acad Sci USA., 1997 Jul. 8; 94(14): 7192-7197).

Representative VEGF Targeting Ligands are provided in FIG. 1. Additional VEGF Targeting Ligands include, but are not limited to, (all cited referenced incorporated herein by reference) the peptide SEQ ID NO: 403 VEPNCDIHVMWEWECFERL-NH₂(Biochemistry 1998, 37, 17754-177764). Additional VEGF Targeting Ligands are provided in, for example, J Med Chem 57: 3011-29 (2014), U.S. Pat. Nos. 9,884,843, 9,446,026, J Med Chem 53: 1686-99 (2010), J Med Chem 48: 8229-36 (2005), J Nat Prod 76: 29-35 (2013), each of which is incorporated herein by reference.

### Transforming Growth Factor-β1 (TGF-β1)

In some embodiments, the Target Extracellular Protein is human transforming growth factor-01 (TGF-β1) (UniProtKB—P01137 (TGFB1_HUMAN)). TGF-β1 is a multifunctional protein that regulates the growth and differentiation of various cell types and is involved in various processes, such as normal development, immune function, microglia function and responses to neurodegeneration. TGF-β1 can promote either T-helper 17 cells (Th17) or regulatory T-cells (Treg) lineage differentiation in a concentration-dependent manner. TGF-β1 expression in the tumor microenvironment has been associated with a poor prognosis, and is implicated in TGF-β1 mediated tumor suppression via T-cell exclusion. TGF-β1 expression has also been implicated in hematological malignancies and fibrosis.

The Protein Data Bank website provides the crystal structure of TGF-β1 searchable by 5E8S, 5E8T, and 5E8U (Tebben, A. J., et al., Acta Crystallogr D Struct Biol., 2016, 72 658-674) 2L5S (Zuniga, J. E., et al, J Mol Biol., 2011, 412 601-618); and 2PJY (Groppe, J., et al., Mol Cell, 2008, 29 157-168); as well as the crystal structure of TGF-β1 bound to various compounds searchable by 5QIK, 5QIL and 5QIM, (Zhang, Y., et al., ACS Med Chem Lett., 2018, 9 1117-1122); 6B8Y (Harikrishnan, L. S., et al., Bioorg Med Chem., 2018, 26 1026-1034); 5E8W, 5E8X, 5E8Z, and 5E90 (Tebben, A. J., et al., Acta Crystallogr D Struct Biol., 2016, 72 658-674); 3TZM (Ogunjimi, A. A. et al., Cell Signal, 2012, 24 476-483); 2X70 (Roth, G. J., et al., J Med Chem., 2010, 53 7287); 3KCF (Guckian, K., et al., Bioorg Med Chem Lett., 2010, 20 326-329); 3FAA (Bonafoux, D., et al., Bioorg Med Chem Lett., 2009, 19 912-916); 1VJY (Gellibert, F, J., et al., J Med Chem., 2004 47 4494-4506); and 1PY5 (Sawyer, J. S., et al., Bioorg Med Chem Lett., 2004, 14 3581-3584). Additionally, Hinck et al., provides insight into the structural studies of the TGF-βs and their receptors and further insight into evolution of the TGF-0 superfamily (Hinck, A., FEBS, 2012, 586(14), 1860-1870).

Representative TGF-β1 Targeting Ligands are provided in FIG. 1. In some embodiments, the TGF-β1 Targeting Ligand is the peptide SEQ ID NO: 404 KRFK peptide (J. Biol. Chem. Vol. 274 (No. 19) pp. 13586-13593 (1999)(incorporated herein by reference). Additional TGF-β1 Targeting Ligands are provided in, for example, Bioorg Med Chem Lett 21: 5642-5 (2011), which is incorporated herein by reference.

### TNF-Alpha (TNF-α)

In some embodiments, the Target Extracellular Protein is human TNF-α (UniProtKB—P01375 (TNFA_HUMAN)). TNF-α is a pro-inflammatory cytokine active in the bodily immune response and serious inflammatory diseases. TNF-α has been implicated in a number of disorders, including but not limited to rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.

The Protein Data Bank website provides the crystal structure of TNF-α searchable by 6RMJ (Valentinis, B., et al., Int. J. Mol. Sci., 2019, 20); 5UUI (Carrington et al., Biophys J., 2017, 113 371-380); 6OOY, 6OOZ and 6OPO (O'Connell, J., et al., Nat. Commun., 2019, 10 5795-5795); and 5TSW (Cha, S. S., J Biol Chem., 1998, 273 2153-2160); as well as the crystal structure of TNF-α bound to various compounds searchable by 5YOY (Ono et al., Protein Sci., 2018, 27 1038-1046); 2AZ5 (He., M. M., et al., Science, 2005, 310: 1022-1025); 5WUX (Lee, J. U., Int J Mol Sci., 2017, 18); 5MU8 (Blevitt et al., J Med Chem., 2017, 60 3511-3517); 4Y60 (Feldman J. L., et al., Biochemistry, 2015, 54 3037-3050); 3WD5 (Hu, S., et al., J Biol Chem, 2013, 288 27059-27067); and 4G3Y (Liang, S. Y., J Biol Chem., 2013, 288 13799-13807).

Representative TNF-α Targeting Ligands are provided in FIG. 1. Additional TNF-α Targeting Ligands can be found in, for example, U.S. Pat. No. 8,541,572; J Chem Inf Model. 2017 May 22; 57(5): 1101-1111; each of which is incorporated by reference herein.

In certain embodiments the TNF-alpha Targeting Ligand is selected from:

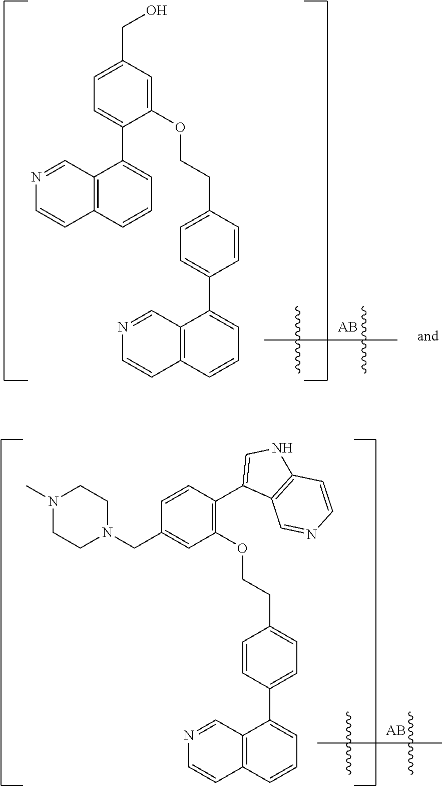

In certain embodiments the TNF-alpha Targeting Ligand is selected from:

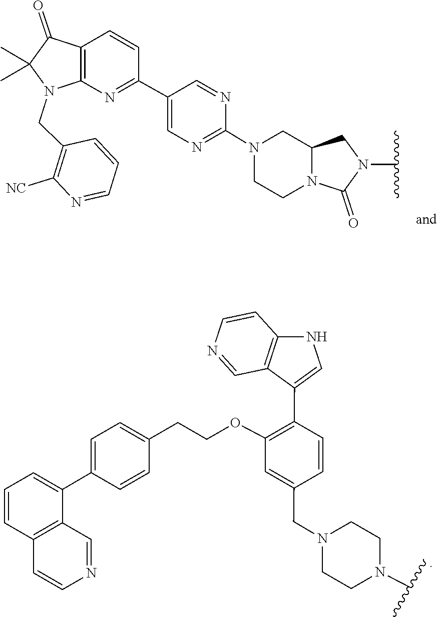 [SMILES: *N1C[C@@H]2CN(c3ncc(-c4ccc5c(n4)N(Cc4cccnc4C#N)C(C)(C)C5=O)cn3)CCN2C1=O ; *N1CCN(Cc2ccc(-c3c[nH]c4ccncc34)c(OCCc3ccc(-c4cccc5ccncc45)cc3)c2)CC1]

In alternative embodiments, the TNF-alpha Targeting Ligand comprises the polypeptide STPTRYS (SEQ ID NO: 405) (Guangdong Yixue 2008, 29(1):55-57). In certain embodiments, the TNF-alpha Targeting Ligand comprises the polypeptide CALWHWWHC SEQ ID NO:121) or C(T/S)WLHWWAC (SEQ ID NO:406) (Diyi Daxue Xuebao 2002, 22(7):597-599). In certain embodiments, the TNF-alpha Targeting Ligand comprises any Tbab protein described in Zhu, et al., 2016, Protein Sci. 25:2066-2075. In certain embodiments, the TNF-alpha Targeting Ligand comprises the polypeptide (L/M)HEL(Y/F)(L/M)X(W/Y/F) (SEQ ID NO:407), as described in Zhang, et al., 2003, Biochem. Biophys. Res. Commun. 310:1181-1187. In certain embodiments, the TNF-alpha Targeting Ligand comprises one of the polypeptides: DHPT-9: D-DDDEK QLKER WYKRW LEYLD EFKKN (SEQ ID NO:408) DHPT-91: D-TEEEK QLKEW WYKHW QEYLE EFKKN (SEQ ID NO:409) (Yang, et al., 2019, FEBS Lett. 593:1292-1302). In certain embodiments, the TNF-alpha Targeting Ligand comprises TNFR1 or TNFR2 (Yang & Yang, 2013, Fenxi Huaxue/Chinese J. Anal. Chem. 41:664-669). In certain embodiments, the TNF-alpha Targeting Ligand comprises anticachexin C1 and/or C2 (Lian, et al., 2013, J. Am. Chem. Soc. 135:11990-11995).

In certain embodiments, the TNF-alpha Targeting Ligand comprises adalimumab, infliximab, etanercept, golimumab, and/or certolizumab. In certain embodiments, the TNF-alpha Targeting Ligand comprises the 29.2 kDa scFv identified in Safarpour, et al., 2018, Iran. J. Pharm. Res. 17:743-752. In certain embodiments, the TNF-alpha Targeting Ligand comprises GACPPCLWQVLCGGSGSGSG (SEQ ID NO:410) (which can be, in a non-limiting example, tris-bromomethyl mesitylene core sulfur linked; Luzi, et al., 2015, Protein Eng. Des. Sel. 28:45-52). In certain embodiments, the TNF-alpha Targeting Ligand comprises any affibodies (˜60 amino acids) identified in Löfdahl, et al., 2009, N. Biotechnol. 26:251-259. In certain embodiments, the TNF-alpha Targeting Ligand comprises any affibodies identified in Kronqvist, et al., 2008, Protein Eng. Des. Sel. 21:247-255. In certain embodiments, the TNF-alpha Targeting Ligand comprises any affibodies identified in Jonsson, et al., 2009, Biotechnol. Appl. Biochem. 54:93-103. In certain embodiments, the TNF-alpha Targeting Ligand comprises the bispecific albumin/TNF binding polypeptide identified in Nilvebrant, et al., 2011, PLoS One 6. In certain embodiments, the TNF-alpha Targeting Ligand comprises the ubiquitin-based artificial binding protein identified in Hoffmann, et al., 2012, PLoS One 7:2-11. In certain embodiments, the TNF-alpha Targeting Ligand comprises HIHDDLLRYYGW linear (SEQ ID NO:411) or tetra branched peptide (SEQ ID NO:412) identified in Brunetti, et al., 2014, Molecules 19:7255-7268.

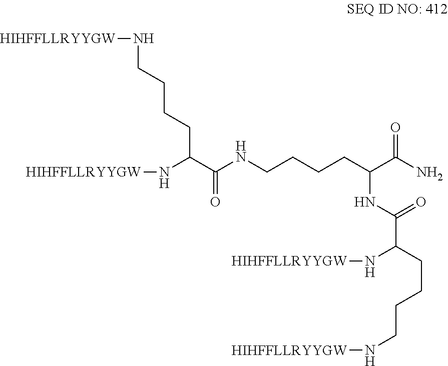

In certain embodiments, the TNF-alpha Targeting Ligand comprises any TNF-binding peptides (P51 and P52) identified in Alizadeh, et al., 2017, Eur. J. Pharm. Sci. 96:490-498.

In certain embodiments, the TNF-alpha Targeting Ligand comprises the scFv antibody identified in Alizadeh, et al., 2015, Adv. Pharm. Bull. 5:661-666. In certain embodiments, the TNF-alpha Targeting Ligand comprises any TNF binding peptide recited in WO 2006/053568 (such as but not limited to KRWSRYF (SEQ ID NO:413), which may in certain embodiments be polyvalent), which is incorporated herein in its entirety by reference. In certain embodiments, the TNF-alpha Targeting Ligand comprises any TNF binding peptide recited in WO 2015/055597 (such as but not limited to HIHDDLLRYYGW (SEQ ID NO:414), which may in certain embodiments be polyvalent), which is incorporated herein in its entirety by reference. In certain embodiments, the TNF-alpha Targeting Ligand comprises YCWSQYLCY (SEQ ID NO:415) as identified in Arthritis & Rheumatism 2007, 56(4):1164-74. In certain embodiments, the TNF-alpha Targeting Ligand comprises DFLPHYKNTSLGHRP (SEQ ID NO:416) as identified in Chirinos-Rojas, et al., 1998, J. Immunol. 161:5621-5626. In certain embodiments, the TNF-alpha Targeting Ligand comprises YCLYQSWCY (SEQ ID NO:417). In certain embodiments, the TNF-alpha Targeting Ligand is its reduced form (i.e., with an internal disulfide bond). In certain embodiments, the TNF-alpha Targeting Ligand is its oxidized form (i.e., without an internal disulfide bond).

In certain embodiments, the TNF binder comprises a compound of formula

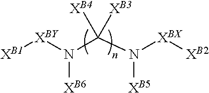

wherein:
X^{B1} and X^{B2} are independently selected from the group consisting of

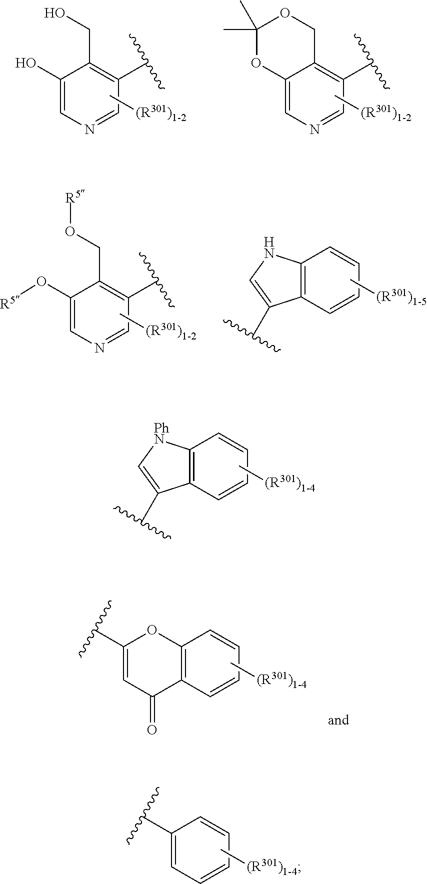

R^{5″} is independently hydrogen or optionally substituted C₁-C₄ alkyl;
X^{B3} and X^{B4} are independently hydrogen or optionally substituted C₁-C₄ alkyl
X^{BX} and X^{BY} are independently carbonyl or CH₂;
n is 2, 3, or 4;
X^{B5} and X^{B6} are independently hydrogen or optionally substituted C₁-C₄ alkyl, or
R³ and R⁴ can combine to form a heterocyclyl ring.

In certain embodiments, the TNF-alpha Targeting Ligand comprises any compound disclosed in U.S. Pat. No. 10,266,532, which is incorporated herein in its entirety by reference. In certain embodiments, the TNF-alpha Targeting Ligand comprises any compound disclosed in U.S. Pat. No. 9,879,016, which is incorporated herein in its entirety by reference. In certain embodiments, the TNF-alpha Targeting Ligand comprises any compound disclosed in WO 2008/142623, which is incorporated herein in its entirety by reference. In certain embodiments, the TNF-alpha Targeting Ligand is

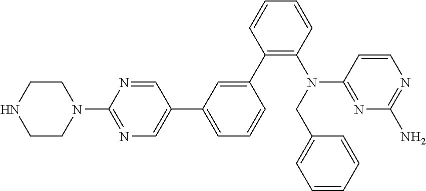 [SMILES: Nc1nccc(N(Cc2ccccc2)c2ccccc2-c2cccc(-c3cnc(N4CCNCC4)nc3)c2)n1]

In certain embodiments, the TNF-alpha Targeting Ligand is of the formula:

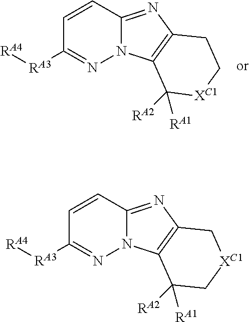

wherein
X^{C1} is CH₂ or O;
R^{A1} is

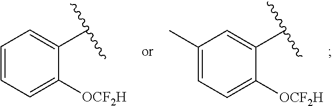

R^{A2} is selected from the group consisting of

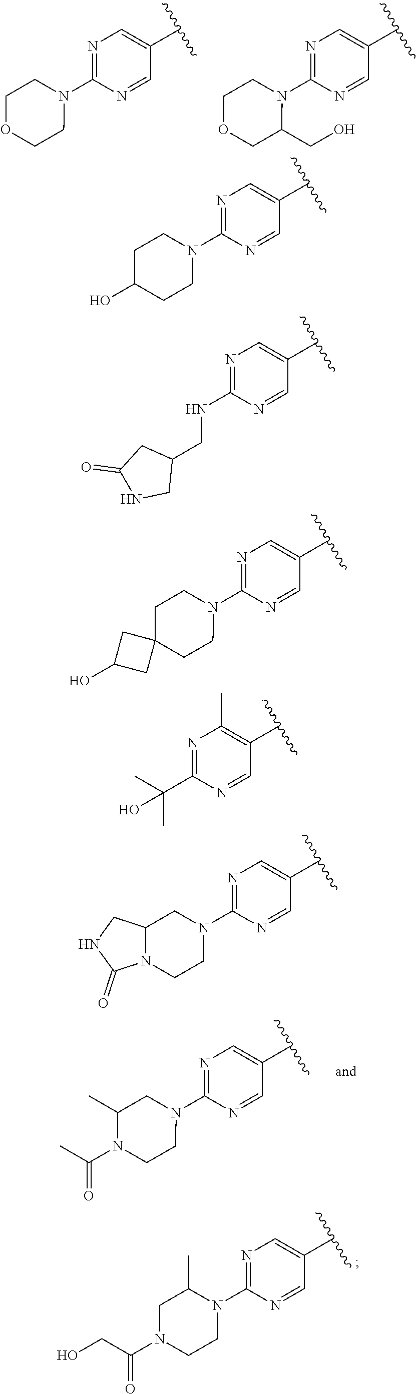

R^{A3} is selected from the group consisting of

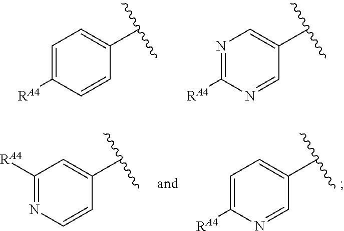

and
R^{A4} is selected from the group consisting of

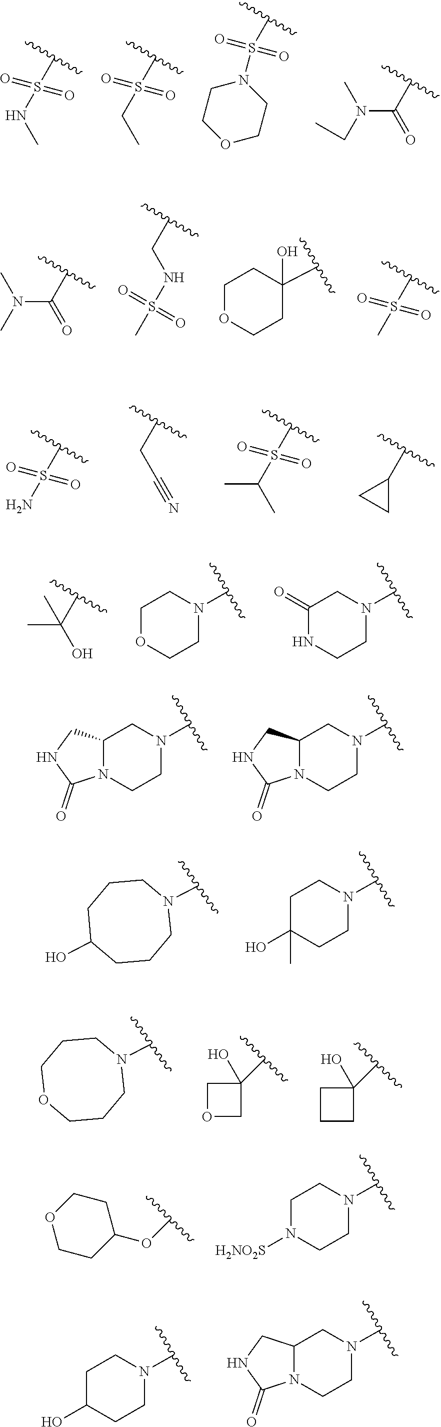

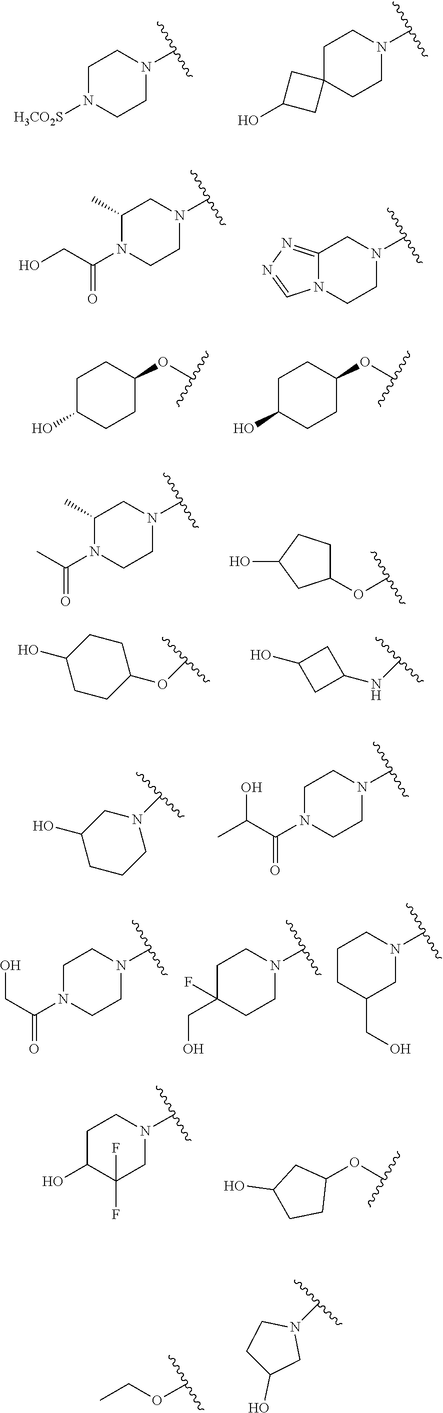

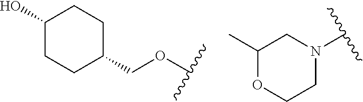

In certain embodiments, the TNF-alpha Targeting Ligand is selected from the group consisting of 2-(5-(1-(2-methoxyphenyl)-2,3-dihydro-1H-benzo[d]pyrrolo[1,2-a]imidazol-7-yl)pyrimidin-2-yl)propan-2-ol; 4-(3-fluorophenyl)-7-(2-morpholinopyrimidin-5-yl)-3,4-dihydro-1H-benzo[4,5]imidazo[2,1-c][1,4]oxazine; (R)-1-phenyl-7-(2-((tetrahydro-2H-pyran-4-yl)oxy)pyridin-4-yl)-2,3-dihydro-1H-benzo[d]pyrrolo[1,2-a]imidazole; (S)-2-(2-morpholinopyrimidin-5-yl)-9-phenyl-8,9-dihydro-6H-pyrido[3′,2′:4,5]imidazo[2,1-c][1,4]oxazine; 2-(5-(8-methyl-8-phenyl-7,8-dihydro-6H-pyrrolo[2′,1′:2,3]imidazo[4,5-b]pyridin-2-yl)pyrimidin-2-yl)propan-2-ol; 2-(5-(1-(tetrahydro-2H-pyran-4-yl)-2,3-dihydro-1H-benzo[d]pyrrolo[1,2-a]imidazol-7-yl)pyrimidin-2-yl)propan-2-ol; (S)-7-(2-((1R,6S)-3,10-diazabicyclo[4.3.1]decan-10-yl)pyrimidin-5-yl)-4-phenyl-3,4-dihydro-1H-benzo[4,5]imidazo[2,1-c][1,4]oxazine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-amine; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one; 7-(5-(8-(2-methoxyphenyl)-7,8-dihydro-6H-cyclopenta[4,5]imidazo[1,2-b]pyridazin-2-yl)pyrimidin-2-yl)hexahydroimidazo[1,5-a]pyrazine-3(2H)-one; 1-(5-(8-phenyl-7,8-dihydro-6H-cyclopenta[4,5]imidazo[1,2-b]pyridazin-2-yl)pyrimidin-2-yl)piperidin-4-ol; 2-(5-(4-(2-methoxyphenyl)-3,4-dihydro-1H-pyran[3′,4′:4,5]imidazo[1,2-a]pyridin-7-yl)pyrimidin-2-yl)propan-2-ol; (S)-7-(5-((R)-8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; (R)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one; 2-(5-(8-(pyridin-2-yl)-7,8-dihydro-6H-pyrrolo[2′,1′:2,3]imidazo[4,5-b]pyridin-2-yl)pyrimidin-2-yl)propan-2-ol; 2-(5-(1-(pyridin-2-yl)-2,3-dihydro-1H-benzo[d]pyrrolo[1,2-a]imidazol-7-yl)pyrimidin-2-yl)propan-2-ol; (S)-7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one; (R)-7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one; 7-(5-(9-(2-methoxyphenyl) 6,7,8,9-tetrahydrobenzo[4,5]imidazo[1,2-a]pyridin-2-yl)pyrimidin-2-yl)-7-azaspiro[3.5]nonan-2-ol; 4-(5-(9-(2-methoxyphenyl)-6,7,8,9-tetrahydrobenzo[4,5]imidazo[1,2-a]pyridin-2-yl)pyrimidin-2-yl)-1,4-oxazepane; 7-(5-(9-(2-methoxyphenyl)-6,7,8,9-tetrahydrobenzo[4,5]imidazo[1,2-a]pyridin-2-yl)pyrimidin-2-yl)hexahydroimidazo[1,5-a]pyrazin-3(2H)-one; 1-(5-(9-(2-methoxyphenyl)-6,7,8,9-tetrahydrobenzo[4,5]imidazo[1,2-a]pyridin-2-yl)pyrimidin-2-yl)-4-methylpiperidin-4-ol; (4-fluoro-1-(5-(9-(2-methoxyphenyl) 6,7,8,9-tetrahydrobenzo[4,5]imidazo[1,2-a]pyridin-2-yl)pyrimidin-2-yl)piperidin-4-yl)methanol; (4-fluoro-1-(5-(9-(2-methoxyphenyl)-6,7,8,9-tetrahydrobenzo[4,5]imidazo[1,2-a]pyridin-2-yl)pyrimidin-2-yl)piperidin-4-yl)methanol; 1-(5-(9-(2-methoxyphenyl)-6,7,8,9-tetrahydrobenzo[4,5]imidazo[1,2-a]pyridin-2-yl)pyrimidin-2-yl)azepan-4-ol; 1-(5-(9-(2-methoxyphenyl)-6,7,8,9-tetrahydrobenzo[4,5]imidazo[1,2-a]pyridin-2-yl)pyrimidin-2-yl)piperidin-4-ol; 1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; 1-(5-(9-(3-fluorophenyl)-8,9-dihydro-6H-pyrido[3′,2′:4,5]imidazo[2,1-c][1,4]oxazin-2-yl)pyrimidin-2-yl)piperidin-4-ol; 7-(5-(8-methyl-8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; 7-(5-(9-(3-fluorophenyl)-8,9-dihydro-6H-pyrido[3′,2′:4,5]imidazo[2,1-c][1,4]oxazin-2-yl)pyrimidin-2-yl)hexahydroimidazo[1,5-a]pyrazin-3(2H)-one; 7-(5-(8-cyclohexyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; 7-(5-((R)-8-cyclohexyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; 7-(5-((S)-4-(2-chlorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one; 7-(5-(4-(3-chlorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one; 7-(5-(4-(2-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one; 4-(5-(1-(2-methoxyphenyl)-2,3-dihydro-1H-cyclopenta[4,5]imidazo[1,2-a]pyridin-7-yl)pyrimidin-2-yl)morpholine; (R)-7-(5-((R)-9-phenyl-8,9-dihydro-6H-pyrano[3′,4′:4,5]imidazo[1,2-b]pyridazin-2-yl)pyrimidin-2-yl)hexahydroimidazo[1,5-a]pyrazin-3(2H)-one; (R)-7-(5-((R)-4-phenyl-3,4-dihydro-1H-pyrano[3′,4′:4,5]imidazo[1,2-a]pyridin-7-yl)pyrimidin-2-yl)hexahydroimidazo[1,5-a]pyrazin-3(2H)-one; 1-(5-(9-(2-methoxyphenyl) 6,7,8,9-tetrahydroimidazo[1,2-a:5,4-b′]dipyridin-2-yl)pyrimidin-2-yl)piperidin-4-ol; 7-(5-(8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; 1-(5-(8-(2-methoxyphenyl)-7,8-dihydro-6H-pyrrolo[2′,1′:2,3]imidazo[4,5-b]pyridin-2-yl)pyrimidin-2-yl)piperidin-4-ol; (S)-1-(5-(9-(2-methoxyphenyl)-8,9-dihydro-6H-pyrido[3′,2′:4,5]imidazo[2,1-c][1,4]oxazin-2-yl)pyrimidin-2-yl)piperidin-4-ol; 7-(5-((S)-9-phenyl-8,9-dihydro-6H-pyrido[3′,2′:4,5]imidazo[2,1-c][1,4]oxazin-2-yl)pyrimidin-2-yl)hexahydroimidazo[1,5-a]pyrazin-1(5H)-one; (S)-1-(5-(9-phenyl-8,9-dihydro-6H-pyrido[3′,2′:4,5]imidazo[2,1-c][1,4]oxazin-2-yl)pyrimidin-2-yl)piperidin-4-ol; (S) 4-(5-(9-phenyl-8,9-dihydro-6H-pyrido[3′,2′:4,5]imidazo[2,1-c][1,4]oxazin-2-yl)pyrimidin-2-yl)piperazin-2-one; (S)-2-(5-(9-phenyl-8,9-dihydro-6H-pyrido[3′,2′:4,5]imidazo[2,1-c][1,4]oxazin-2-yl)pyrimidin-2-yl)propan-2-ol; (S)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one; (R)-3-(5-(1-phenyl-2,3-dihydro-1H-benzo[d]pyrrolo[1,2-a]imidazol-7-yl)pyrimidin-2-yl)oxetan-3-ol; (R)-1-(5-(1-phenyl-2,3-dihydro-1H-benzo[d]pyrrolo[1,2-a]imidazol-7-yl)pyrimidin-2-yl)cyclobutanol; (R)-4-(5-(1-phenyl-2,3-dihydro-1H-benzo[d]pyrrolo[1,2-a]imidazol-7-yl)pyrimidin-2-yl)tetrahydro-2H-pyran-4-ol; (R)-7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one; 2-(5-(4-(2,6-dichlorophenyl)-3,4-dihydro-1H-benzo[4,5]imidazo[2,1-c][1,4]oxazin-7-yl)pyrimidin-2-yl)propan-2-ol; (S)-2-(5-(4-(2-methoxyphenyl)-3,4-dihydro-1H-benzo[4,5]imidazo[2,1-c][1,4]oxazin-7-yl)pyrimidin-2-yl)propan-2-ol; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one; 7-(5-(4-(2-chlorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one; 7-(5-(1,2′,3,3′-tetrahydrospiro[benzo[4,5]imidazo[2,1-c][1,4]oxazine-4,1′-inden]-7-yl)pyrimidin-2-yl)hexahydroimidazo[1,5-a]pyrazin-3(2H)-one; (S)-2-hydroxy-1-(4-(5-(4-phenyl-3,4-dihydro-1H-benzo[4,5]imidazo[2,1-c][1,4]oxazin-7-yl)pyrimidin-2-yl)piperazin-1-yl)ethanone; 7-(5-((S) 4-phenyl-3,4-dihydro-1H-benzo[4,5]imidazo[2,1-c][1,4]oxazin-7-yl)pyrimidin-2-yl)-7-azaspiro[3.5]nonan-1-ol; (R)-1-(5-(8-(3-fluorophenyl)-7,8-dihydro-6H-pyrrolo[2′,1′:2,3]imidazo[4,5-b]pyridin-2-yl)pyrimidin-2-yl)piperidin-4-ol; (R)-1-(5-(8-phenyl-7,8-dihydro-6H-pyrrolo[2′,1′:2,3]imidazo[4,5-b]pyridin-2-yl)pyrimidin-2-yl)piperidin-4-ol; (R) 4-(5-(8-phenyl-7,8-dihydro-6H-pyrrolo[2′,1′:2,3]imidazo[4,5-b]pyridin-2-yl)pyrimidin-2-yl)morpholine; (S)-2-(5-(1-(2,5-dimethylphenyl)-2,3-dihydro-1H-benzo[d]pyrrolo[1,2-a]imidazol-7-yl)pyrimidin-2-yl)propan-2-ol; (R)-2-(5-(1-(2,5-dimethylphenyl)-2,3-dihydro-1H-benzo[d]pyrrolo[1,2-a]imidazol-7-yl)pyrimidin-2-yl)propan-2-ol; (S)-4-(3-fluorophenyl)-7-(2-morpholinopyrimidin-5-yl)-3,4-dihydro-1H-benzo[4,5]imidazo[2,1-c][1,4]oxazine; 7-(5-(8-(2,5-dimethylphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one compound with ethane (1:1); 7-(5-(8-(3-fluorophenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; (R)-1-(5-(1-phenyl-2,3-dihydro-1H-benzo[d]pyrrolo[1,2-a]imidazol-7-yl)pyrimidin-2-yl)piperidin-4-ol; (R)-4-(5-(1-phenyl-2,3-dihydro-1H-benzo[d]pyrrolo[1,2-a]imidazol-7-yl)pyrimidin-2-yl)thiomorpholine 1,1-dioxide; -(5-(8-phenyl-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; 7-(5-((R)-1-phenyl-2,3-dihydro-1H-benzo[d]pyrrolo[1,2-a]imidazol-7-yl)pyrimidin-2-yl)hexahydroimidazo[1,5-a]pyrazin-3(2H)-one; 3,3-difluoro-1-(5-((R)-1-phenyl-2,3-dihydro-1H-benzo[d]pyrrolo[1,2-a]imidazol-7-yl)pyrimidin-2-yl)piperidin-4-ol; (S)-2-(5-(4-(2-(difluoromethoxy)phenyl)-3,4-dihydro-1H-benzo[4,5]imidazo[2,1-c][1,4]oxazin-7-yl)pyrimidin-2-yl)propan-2-ol; (S)-2-(5-(9-(2-methoxyphenyl)-6,7,8,9-tetrahydrobenzo[4,5]imidazo[1,2-a]pyridin-2-yl)pyrimidin-2-yl)propan-2-ol; (R)-7-(5-(1-phenyl-2,3-dihydro-1H-benzo[d]pyrrolo[1,2-a]imidazol-7-yl)pyrimidin-2-yl) 7-azaspiro[3.5]nonan-2-ol; 1-(5-(8-(2,5-dimethylphenyl)-7,8-dihydro-6H-pyrrolo[2′,1′:2,3]imidazo[4,5-b]pyridin-2-yl)pyrimidin-2-yl)piperidin-4-ol; 4-(5-(10-(2-methoxyphenyl)-7,8,9,10-tetrahydro-6H-cyclohepta[4,5]imidazo[1,2-a]pyridin-2-yl)pyrimidin-2-yl)morpholine; (R)-4-(5-(1-phenyl-2,3-dihydro-1H-benzo[d]pyrrolo[1,2-a]imidazol-7-yl)pyrimidin-2-yl)morpholine; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one; 4-(5-(8-(2,5-dimethylphenyl)-7,8-dihydro-6H-pyrrolo[2′,1′:2,3]imidazo[4,5-b]pyridin-2-yl)pyrimidin-2-yl)piperazin-2-one; 2-(5-(1-(2,5-dimethylphenyl)-2,3-dihydro-1H-benzo[d]pyrrolo[1,2-a]imidazol-7-yl)pyrimidin-2-yl)propan-2-ol; 3,3-difluoro-1-(5-((S)-4-phenyl-3,4-dihydro-1H-benzo[4,5]imidazo[2,1-c][1,4]oxazin-7-yl)pyrimidin-2-yl)piperidin-4-ol; 7-(5-(4-(3-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one; (R)-2-(5-(1-(2-methoxyphenyl)-2,3-dihydro-1H-cyclopenta[4,5]imidazo[1,2-a]pyridin-7-yl)pyrimidin-2-yl)propan-2-ol; 7-(4-(isopropylsulfonyl)phenyl)-1-phenyl-2,3-dihydro-1H-benzo[d]pyrrolo[1,2-a]imidazole; 2-(5-(9-(2-methoxyphenyl)-6,7,8,9-tetrahydrobenzo[4,5]imidazo[1,2-a]pyridin-2-yl)pyrimidin-2-yl)propan-2-ol; 4-(5-(8-(2,5-dimethylphenyl)-7,8-dihydro-6H-pyrrolo[2′,1′:2,3]imidazo[4,5-b]pyridin-2-yl)pyrimidin-2-yl)morpholine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 4-(5-(9-(2-methoxyphenyl)-6,7,8,9-tetrahydrobenzo[4,5]imidazo[1,2-a]pyridin-2-yl)pyrimidin-2-yl)morpholine; (S)-1-(5-(4-phenyl-3,4-dihydro-1H-benzo[4,5]imidazo[2,1-c][1,4]oxazin-7-yl)pyrimidin-2-yl)piperidin-4-ol; 1-(5-(9-phenyl-6,7,8,9-tetrahydroimidazo[1,2-a:5,4-b′]dipyridin-2-yl)pyrimidin-2-yl)piperidin-4-ol; (R)-2-(5-(1-phenyl-2,3-dihydro-1H-benzo[d]pyrrolo[1,2-a]imidazol-7-yl)pyrimidin-2-yl)propan-2-ol; 4-(5-(8-(3-fluorophenyl)-7,8-dihydro-6H-pyrrolo[2′,1′:2,3]imidazo[4,5-b]pyridin-2-yl)pyrimidin-2-yl)morpholine; 2-(5-(10-(2-methoxyphenyl)-7,8,9,10-tetrahydro-6H-cyclohepta[4,5]imidazo[1,2-a]pyridin-2-yl)pyrimidin-2-yl)propan-2-ol; N-methyl-4-(1-phenyl-2,3-dihydro-1H-benzo[d]pyrrolo[1,2-a]imidazol-7-yl)benzenesulfonamide; 1-(5-(8-(3-fluorophenyl)-7,8-dihydro-6H-pyrrolo[2′,1′:2,3]imidazo[4,5-b]pyridin-2-yl)pyrimidin-2-yl)piperidin-4-ol; 7-(4-(ethylsulfonyl)phenyl)-1-phenyl-2,3-dihydro-1H-benzo[d]pyrrolo[1,2-a]imidazole; (S)-7-(2-morpholinopyrimidin-5-yl)-4-phenyl-3,4-dihydro-1H-benzo[4,5]imidazo[2,1-c][1,4]oxazine; 4-(5-(8-phenyl-7,8-dihydro-6H-pyrrolo[2′,1′:2,3]imidazo[4,5-b]pyridin-2-yl)pyrimidin-2-yl)morpholine; 4-(5-(8-phenyl-7,8-dihydro-6H-pyrrolo[2′,1′:2,3]imidazo[4,5-b]pyridin-2-yl)pyrimidin-2-yl)morpholine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 1-(5-(8-phenyl-7,8-dihydro-6H-pyrrolo[2′,1′:2,3]imidazo[4,5-b]pyridin-2-yl)pyrimidin-2-yl)piperidin-4-ol; (S)-7-(2-(1,4-oxazepan-4-yl)pyrimidin-5-yl)-4-phenyl-3,4-dihydro-1H-benzo[4,5]imidazo[2,1-c][1,4]oxazine; 4-(5-(1-phenyl-2,3-dihydro-1H-benzo[d]pyrrolo[1,2-a]imidazol-7-yl)pyrimidin-2-yl)morpholine; 3,3-difluoro-1-(5-(4-(3-fluorophenyl)-3,4-dihydro-1H-benzo[4,5]imidazo[2,1-c][1,4]oxazin-7-yl)pyrimidin-2-yl)piperidin-4-ol; (1-(5-((S)-4-phenyl-3,4-dihydro-1H-benzo[4,5]imidazo[2,1-c][1,4]oxazin-7-yl)pyrimidin-2-yl)piperidin-3-yl)methanol; 1-(5-((S)-4-phenyl-3,4-dihydro-1H-benzo[4,5]imidazo[2,1-c][1,4]oxazin-7-yl)pyrimidin-2-yl)azepan-4-ol; (S)-4-(5-(4-phenyl-3,4-dihydro-1H-benzo[4,5]imidazo[2,1-c][1,4]oxazin-7-yl)pyrimidin-2-yl)piperazine-1-sulfonamide; N-(4-(1-phenyl-2,3-dihydro-1H-benzo[d]pyrrolo[1,2-a]imidazol-7-yl)benzyl)methanesulfonamide; 2-(5-(1-(2-methoxyphenyl)-2,3-dihydro-1H-cyclopenta[4,5]imidazo[1,2-a]pyridin-7-yl)pyrimidin-2-yl)propan-2-ol; 7-(4-(methylsulfonyl)phenyl)-1-phenyl-2,3-dihydro-1H-benzo[d]pyrrolo[1,2-a]imidazole; 7-(2-(1,4-oxazepan-4-yl)pyrimidin-5-yl)-4-(3-fluorophenyl)-3,4-dihydro-1H-benzo[4,5]imidazo[2,1-c][1,4]oxazine; 4-(1-phenyl-2,3-dihydro-1H-benzo[d]pyrrolo[1,2-a]imidazol-7-yl)benzenesulfonamide; (4-(5-((S)-4-phenyl-3,4-dihydro-1H-benzo[4,5]imidazo[2,1-c][1,4]oxazin-7-yl)pyrimidin-2-yl)morpholin-2-yl)methanol; (S)-4-(5-(4-phenyl-3,4-dihydro-1H-benzo[4,5]imidazo[2,1-c][1,4]oxazin-7-yl)pyrimidin-2-yl)thiomorpholine 1,1-dioxide; 4-(5-(9-phenyl-6,7,8,9-tetrahydroimidazo[1,2-a:5,4-b′]dipyridin-2-yl)pyrimidin-2-yl)morpholine; (R) 4-(5-(1-phenyl-1,2,3,4-tetrahydrobenzo[4,5]imidazo[1,2-a]pyridin-8-yl)pyrimidin-2-yl)morpholine; 2-(5-(1-(3-fluorophenyl)-2,3-dihydro-1H-benzo[d]pyrrolo[1,2-a]imidazol-7-yl)pyrimidin-2-yl)propan-2-ol; (S)-(4-fluoro-1-(5-(4-phenyl-3,4-dihydro-1H-benzo[4,5]imidazo[2,1-c][1,4]oxazin-7-yl)pyrimidin-2-yl)piperidin-4-yl)methanol; 4-(5-(9-(3-chlorophenyl)-6,7,8,9-tetrahydrobenzo[4,5]imidazo[1,2-a]pyridin-2-yl)pyrimidin-2-yl)morpholine; (R)-2-(5-(1-phenyl-1,2,3,4-tetrahydrobenzo[4,5]imidazo[1,2-a]pyridin-8-yl)pyrimidin-2-yl)propan-2-ol; (S)-7-(2-(1′-methyl-[4,4′-bipiperidin]-1-yl)pyrimidin-5-yl)-4-phenyl-3,4-dihydro-1H-benzo[4,5]imidazo[2,1-c][1,4]oxazine; 2-(5-(9-phenyl-6,7,8,9-tetrahydroimidazo[1,2-a:5,4-b′]dipyridin-2-yl)pyrimidin-2-yl)propan-2-ol; 7-(2-Morpholinopyrimidin-5-yl)-4-phenyl-3,4-dihydro-1H-benzo[4,5]imidazo[2,1-c][1,4]oxazine; (4S)-7-(2-(2-methylmorpholino)pyrimidin-5-yl)-4-phenyl-3,4-dihydro-1H-benzo[4,5]imidazo[2,1-c][1,4]oxazine; 7-(5-(4-(3-fluorophenyl)-3,4-dihydro-1H-benzo[4,5]imidazo[2,1-c][1,4]oxazin-7-yl)pyrimidin-2-yl)-7-azaspiro[3.5]nonan-2-ol; 4-(5-(1-phenyl-1,2,3,4-tetrahydrobenzo[4,5]imidazo[1,2-a]pyridin-8-yl)pyrimidin-2-yl)morpholine; Ethyl 2-[[5-[9-(2-methoxyphenyl)-6,7,8,9-tetrahydropyrido[1,2-a]benzimidazol-2-yl]pyrimidin-2-yl]amino]acetate; (S)-7-(2-(5,6-dihydro-[1,2,4]triazolo[4,3-a]pyrazin-7(8H)-yl)pyrimidin-5-yl)-4-phenyl-3,4-dihydro-1H-benzo[4,5]imidazo[2,1-c][1,4]oxazine; 2-(5-(8-(3-fluorophenyl) 7,8-dihydro-6H-pyrrolo[2′,1′:2,3]imidazo[4,5-b]pyridin-2-yl)pyrimidin-2-yl)propan-2-ol; (S)-7-(2-(4-(methylsulfonyl)piperazin-1-yl)pyrimidin-5-yl)-4-phenyl-3,4-dihydro-1H-benzo[4,5]imidazo[2,1-c][1,4]oxazine; 2-(5-(8-phenyl-7,8-dihydro-6H-pyrrolo[2′,1′:2,3]imidazo[4,5-b]pyridin-2-yl)pyrimidin-2-yl)propan-2-ol; 2-(4-(1-phenyl-2,3-dihydro-1H-benzo[d]pyrrolo[1,2-a]imidazol-7-yl)phenyl)acetonitrile; 4-(5-(8-phenyl-7,8-dihydro-6H-pyrrolo[2′,1′:2,3]imidazo[4,5-b]pyridin-2-yl)pyrimidin-2-yl)piperazin-2-one; 7-(2-cyclopropylpyrimidin-5-yl)-1-phenyl-2,3-dihydro-1H-benzo[d]pyrrolo[1,2-a]imidazole; (S)-4-(5-(4-phenyl-3,4-dihydro-1H-benzo[4,5]imidazo[2,1-c][1,4]oxazin-7-yl)pyrimidin-2-yl)piperazin-2-one; 2-((5-(9-(2-methoxyphenyl)-6,7,8,9-tetrahydrobenzo[4,5]imidazo[1,2-a]pyridin-2-yl)pyridin-2-yl)oxy)acetic acid; 7-(6-(ethylsulfonyl)pyridin-3-yl)-1-phenyl-2,3-dihydro-1H-benzo[d]pyrrolo[1,2-a]imidazole; 4-(5-(8-(3-fluorophenyl)-7,8-dihydro-6H-pyrrolo[2′,1′:2,3]imidazo[4,5-b]pyridin-2-yl)pyrimidin-2-yl)piperazin-2-one; 10-(3-fluorophenyl)-2-(2-morpholinopyrimidin-5-yl)-7,8,9,10-tetrahydro-6H-cyclohepta[4,5]imidazo[1,2-a]pyridin-10-ol; 4-(5-(9-phenyl-6,7,8,9-tetrahydroimidazo[1,2-a:5,4-b′]dipyridin-2-yl)pyrimidin-2-yl)piperazin-2-one; (S)-6-(5-(4-phenyl-3,4-dihydro-1H-benzo[4,5]imidazo[2,1-c][1,4]oxazin-7-yl)pyrimidin-2-yl)-6-azaspiro[3.4]octan-2-ol; N,N-dimethyl-4-(1-phenyl-2,3-dihydro-1H-benzo[d]pyrrolo[1,2-a]imidazol-7-yl)benzamide; N-ethyl-N-methyl-4-(1-phenyl-2,3-dihydro-1H-benzo[d]pyrrolo[1,2-a]imidazol-7-yl)benzamide; 7-(6-morpholinopyridin-3-yl)-4-phenyl-3,4-dihydro-1H-benzo[4,5]imidazo[2,1-c][1,4]oxazine; 2-(5-(1-phenyl-1,2,3,4-tetrahydrobenzo[4,5]imidazo[1,2-a]pyridin-8-yl)pyrimidin-2-yl)propan-2-ol; 2-(5-(1-Phenyl-2,3-dihydro-1H-benzo[d]pyrrolo[1,2-a]imidazol-7-yl)pyrimidin-2-yl)propan-2-ol; (S)-4-(2-hydroxyethyl)-1-(5-(4-phenyl-3,4-dihydro-1H-benzo[4,5]imidazo[2,1-c][1,4]oxazin-7-yl)pyrimidin-2-yl)piperidin-4-ol; (S)-7-(2-(2-oxa-7-azaspiro[3.5]nonan-7-yl)pyrimidin-5-yl)-4-phenyl-3,4-dihydro-1H-benzo[4,5]imidazo[2,1-c][1,4]oxazine; 2-(1-(5-((S)-4-phenyl-3,4-dihydro-1H-benzo[4,5]imidazo[2,1-c][1,4]oxazin-7-yl)pyrimidin-2-yl)piperidin-3-yl)acetic acid; 7-(5-methyl-6-morpholinopyridin-3-yl)-4-phenyl-3,4-dihydro-1H-benzo[4,5]imidazo[2,1-c][1,4]oxazine; 7-(5-(2-methyl-1H-imidazol-1-yl)pyrazin-2-yl)-4-phenyl-3,4-dihydro-1H-benzo[4,5]imidazo[2,1-c][1,4]oxazine; 2-(5-(1-cyclohexyl-2,3-dihydro-1H-benzo[d]pyrrolo[1,2-a]imidazol-7-yl)pyrimidin-2-yl)propan-2-ol; 2-(5-(4-phenyl-3,4-dihydro-1H-benzo[4,5]imidazo[2,1-c][1,4]oxazin-7-yl)pyrimidin-2-yl)propan-2-ol; (S)-(4-(methylsulfonyl) 1-(5-(4-phenyl-3,4-dihydro-1H-benzo[4,5]imidazo[2,1-c][1,4]oxazin-7-yl)pyrimidin-2-yl)piperidin-4-yl)methanol; 1-(1-(5-((S)-4-phenyl-3,4-dihydro-1H-benzo[4,5]imidazo[2,1-c][1,4]oxazin-7-yl)pyrimidin-2-yl)piperidin-4-yl)ethanol; (S)-4-(5-(4-phenyl-3,4-dihydro-1H-benzo[4,5]imidazo[2,1-c][1,4]oxazin-7-yl)pyrimidin-2-yl)-1,4-diazepan-2-one; 2-(4-(1-phenyl-2,3-dihydro-1H-benzo[d]pyrrolo[1,2-a]imidazol-7-yl)phenoxy)acetonitrile; (S)—N-(1-(5-(4-phenyl-3,4-dihydro-1H-benzo[4,5]imidazo[2,1-c][1,4]oxazin-7-yl)pyrimidin-2-yl)piperidin-4-yl)methanesulfonamide; (S)-3-(1-(5-(4-phenyl-3,4-dihydro-1H-benzo[4,5]imidazo[2,1-c][1,4]oxazin-7-yl)pyrimidin-2-yl)piperidin-4-yl)propanoic acid; 4-phenyl-7-(6-(trifluoromethyl)pyridin-3-yl)-3,4-dihydro-1H-benzo[4,5]imidazo[2,1-c][1,4]oxazine; 4-(5-(1-phenyl-1,2,3,4-tetrahydrobenzo[4,5]imidazo[1,2-a]pyridin-8-yl)pyrimidin-2-yl)piperazin-2-one; 7-(5-fluoro-6-methoxypyridin-3-yl)-4-phenyl-3,4-dihydro-1H-benzo[4,5]imidazo[2,1-c][1,4]oxazine; N,N-dimethyl-5-(1-phenyl-2,3-dihydro-1H-benzo[d]pyrrolo[1,2-a]imidazol-7-yl)pyridin-2-amine; 7-(2-methylpyridin-4-yl)-1-phenyl-2,3-dihydro-1H-benzo[d]pyrrolo[1,2-a]imidazole; (4S)-4-phenyl-7-(2-(2-(trifluoromethyl)morpholino)pyrimidin-5-yl)-3,4-dihydro-1H-benzo[4,5]imidazo[2,1-c][1,4]oxazine; 7-(5-((S)-4-phenyl-3,4-dihydro-1H-benzo[4,5]imidazo[2,1-c][1,4]oxazin-7-yl)pyrimidin-2-yl)-2,7-diazaspiro[4.4]nonan-1-one; N-cyclopentyl-5-(4-phenyl-3,4-dihydro-1H-benzo[4,5]imidazo[2,1-c][1,4]oxazin-7-yl)pyrimidin-2-amine; 7-(2-(1H-pyrazol-1-yl)pyrimidin-5-yl)-4-phenyl-3,4-dihydro-1H-benzo[4,5]imidazo[2,1-c][1,4]oxazine; (4S)-7-(2-(2,6-dimethylmorpholino)pyrimidin-5-yl)-4-phenyl-3,4-dihydro-1H-benzo[4,5]imidazo[2,1-c][1,4]oxazine; 7-(6-methylpyridin-3-yl)-1-phenyl-2,3-dihydro-1H-benzo[d]pyrrolo[1,2-a]imidazole; 7-(5-ethoxypyridin-3-yl)-1-phenyl-2,3-dihydro-1H-benzo[d]pyrrolo[1,2-a]imidazole; 2-(2-morpholinopyrimidin-5-yl)-9-(m-tolyl) 6,7,8,9-tetrahydrobenzo[4,5]imidazo[1,2-a]pyridin-9-ol; 7-(6-(methylthio)pyridin-3-yl)-1-phenyl-2,3-dihydro-1H-benzo[d]pyrrolo[1,2-a]imidazole; ethyl 2-((5-(10-(2-methoxyphenyl) 7,8,9,10-tetrahydro-6H-cyclohepta[4,5]imidazo[1,2-a]pyridin-2-yl)pyrimidin-2-yl)amino)acetate; (S)-3-(4-(5-(4-phenyl-3,4-dihydro-1H-benzo[4,5]imidazo[2,1-c][1,4]oxazin-7-yl)pyrimidin-2-yl)piperazin-1-yl)propan-1-ol; 9-(3-fluorophenyl)-2-(2-morpholinopyrimidin-5-yl)-6,7,8,9-tetrahydrobenzo[4,5]imidazo[1,2-a]pyridin-9-ol; 2-(2-morpholinopyrimidin-5-yl)-9-phenyl-6,7,8,9-tetrahydrobenzo[4,5]imidazo[1,2-a]pyridin-9-ol; 4-(2,5-difluorophenyl)-7-(2-morpholinopyrimidin-5-yl)-3,4-dihydro-1H-benzo[4,5]imidazo[2,1-c][1,4]oxazine; 1-phenyl-7-(6-(piperazin-1-yl)pyridin-3-yl)-2,3-dihydro-1H-benzo[d]pyrrolo[1,2-a]imidazole; 9-(2-Methoxyphenyl)-2-(2-morpholinopyrimidin-5-yl)-6,7,8,9-tetrahydrobenzo[4,5]imidazo[1,2-a]pyridin-9-ol; (S)-7-(2-(2-oxa-6-azaspiro[3.4]octan-6-yl)pyrimidin-5-yl)-4-phenyl-3,4-dihydro-1H-benzo[4,5]imidazo[2,1-c][1,4]oxazine; 7-(5-(1H-imidazol-1-yl)pyrazin-2-yl)-4-phenyl-3,4-dihydro-1H-benzo[4,5]imidazo[2,1-c][1,4]oxazine; 7-(furo[3,2-b]pyridin-6-yl)-4-phenyl-3,4-dihydro-1H-benzo[4,5]imidazo[2,1-c][1,4]oxazine; 10-(3-chlorophenyl)-2-(2-morpholinopyrimidin-5-yl)-7,8,9,10-tetrahydro-6H-cyclohepta[4,5]imidazo[1,2-a]pyridin-10-ol; N-ethyl-4-(1-phenyl-2,3-dihydro-1H-benzo[d]pyrrolo[1,2-a]imidazol-7-yl)benzamide; 2-(3-(1-phenyl-2,3-dihydro-1H-benzo[d]pyrrolo[1,2-a]imidazol-7-yl)phenoxy)acetonitrile; 2-(2-morpholinopyrimidin-5-yl)-10-(m-tolyl)-7,8,9,10-tetrahydro-6H-cyclohepta[4,5]imidazo[1,2-a]pyridin-10-ol; 2-((5-(10-(2-methoxyphenyl)-7,8,9,10-tetrahydro-6H-cyclohepta[4,5]imidazo[1,2-a]pyridin-2-yl)pyrimidin-2-yl)amino)acetic acid; N-cyclopropyl-4-(1-phenyl-2,3-dihydro-1H-benzo[d]pyrrolo[1,2-a]imidazol-7-yl)benzamide; 4-(1-phenyl-2,3-dihydro-1H-benzo[d]pyrrolo[1,2-a]imidazol-7-yl)benzamide; 1-(4-(5-(4-phenyl-3,4-dihydro-1H-benzo[4,5]imidazo[2,1-c][1,4]oxazin-7-yl)pyridin-2-yl)piperazin-1-yl)ethanone; 7-(6-(4-methylpiperazin-1-yl)pyridin-3-yl)-1-phenyl-2,3-dihydro-1H-benzo[d]pyrrolo[1,2-a]imidazole; 7-(benzo[d][1,3]dioxol-5-yl)-1-phenyl-2,3-dihydro-1H-benzo[d]pyrrolo[1,2-a]imidazole; 9-(3-fluoro-2-methylphenyl)-2-(2-morpholinopyrimidin-5-yl)-6,7,8,9-tetrahydrobenzo[4,5]imidazo[1,2-a]pyridin-9-ol; 8-Phenyl-2-(4-(pyrimidin-2-yl)piperazin-1-yl) 7,8-dihydro-6H-pyrrolo[2′,1′:2,3]imidazo[4,5-b]pyridine; 9-(4-fluorophenyl)-2-(2-morpholinopyrimidin-5-yl)-6,7,8,9-tetrahydrobenzo[4,5]imidazo[1,2-a]pyridin-9-ol; 7-(5-((S)-4-phenyl-3,4-dihydro-1H-benzo[4,5]imidazo[2,1-c][1,4]oxazin-7-yl)pyrimidin-2-yl)tetrahydro-1H-oxazolo[3,4-a]pyrazin-3(5H)-one; 2-((5-(9-(2-methoxyphenyl)-6,7,8,9-tetrahydrobenzo[4,5]imidazo[1,2-a]pyridin-2-yl)pyrimidin-2-yl)amino)acetic acid; 10-(4-fluorophenyl)-2-(2-morpholinopyrimidin-5-yl)-7,8,9,10-tetrahydro-6H-cyclohepta[4,5]imidazo[1,2-a]pyridin-10-ol; 9-(3-chlorophenyl)-2-(2-morpholinopyrimidin-5-yl)-6,7,8,9-tetrahydrobenzo[4,5]imidazo[1,2-a]pyridin-9-ol; N-cyclopropyl-5-(4-phenyl-3,4-dihydro-1H-benzo[4,5]imidazo[2,1-c][1,4]oxazin-7-yl)pyrimidin-2-amine; 9-(3-chloro-5-fluorophenyl)-2-(2-morpholinopyrimidin-5-yl)-6,7,8,9-tetrahydrobenzo[4,5]imidazo[1,2-a]pyridin-9-ol; N,N-dimethyl-5-(4-phenyl-3,4-dihydro-1H-benzo[4,5]imidazo[2,1-c][1,4]oxazin-7-yl)pyrimidin-2-amine; 1-(5-(1-phenyl-1,2,3,4-tetrahydrobenzo[4,5]imidazo[1,2-a]pyridin-8-yl)pyrimidin-2-yl)piperidine-4-carboxylic acid; 7-(6-isopropoxypyridin-3-yl)-4-phenyl-3,4-dihydro-1H-benzo[4,5]imidazo[2,1-c][1,4]oxazine; 7-(6-isopropoxypyridin-3-yl)-4-phenyl-3,4-dihydro-1H-benzo[4,5]imidazo[2,1-c][1,4]oxazine; 4-(5-(6-phenyl-7,8-dihydro-6H-pyrrolo[1′,2′:1,2]imidazo[4,5-c]pyridin-3-yl)pyrimidin-2-yl)morpholine; 1-phenyl-7-(1-(pyridin-3-ylmethyl)-1H-pyrazol-4-yl)-2,3-dihydro-1H-benzo[d]pyrrolo[1,2-a]imidazole; 5-(4-phenyl-3,4-dihydro-1H-benzo[4,5]imidazo[2,1-c][1,4]oxazin-7-yl)picolinonitrile; 7-(4-methyl-3,4-dihydro-2H-pyrido[3,2-b][1,4]oxazin-7-yl)-4-phenyl-3,4-dihydro-1H-benzo[4,5]imidazo[2,1-c][1,4]oxazine; 2-(2-morpholinopyrimidin-5-yl)-9-(p-tolyl)-6,7,8,9-tetrahydrobenzo[4,5]imidazo[1,2-a]pyridin-9-ol; 7-(6-(methylsulfonyl)pyridin-3-yl)-4-phenyl-3,4-dihydro-1H-benzo[4,5]imidazo[2,1-c][1,4]oxazine; N-(2-methoxyethyl)-4-(1-phenyl-2,3-dihydro-1H-benzo[d]pyrrolo[1,2-a]imidazol-7-yl)benzamide; N-methyl-4-(1-phenyl-2,3-dihydro-1H-benzo[d]pyrrolo[1,2-a]imidazol-7-yl)benzamide; 4-phenyl-7-(6-(2,2,2-trifluoroethoxy)pyridin-3-yl)-3,4-dihydro-1H-benzo[4,5]imidazo[2,1-c][1,4]oxazine; 1-(5-(4-phenyl-3,4-dihydro-1H-benzo[4,5]imidazo[2,1-c][1,4]oxazin-7-yl)pyrimidin-2-yl)piperidine-4-carboxylic acid; 7-(5-methyl-6-(4-methylpiperazin-1-yl)pyridin-3-yl)-4-phenyl-3,4-dihydro-1H-benzo[4,5]imidazo[2,1-c][1,4]oxazine; 5-(9-(2-Methoxyphenyl)-6,7,8,9-tetrahydrobenzo[4,5]imidazo[1,2-a]pyridin-2-yl)thiophene-2-carboxylic acid; 7-(6-(4-methylpiperazin-1-yl)pyridin-3-yl)-4-phenyl-3,4-dihydro-1H-benzo[4,5]imidazo[2,1-c][1,4]oxazine; (S)-7-(2-(1′-methyl-[4,4′-bipiperidin]-1-yl)pyrimidin-5-yl)-4-phenyl-3,4-dihydro-1H-benzo[4,5]imidazo[2,1-c][1,4]oxazine; 7-(2-methoxypyrimidin-5-yl)-4-phenyl-3,4-dihydro-1H-benzo[4,5]imidazo[2,1-c][1,4]oxazine; 10-(3-chloro-5-fluorophenyl)-2-(2-morpholinopyrimidin-5-yl)-7,8,9,10-tetrahydro-6H-cyclohepta[4,5]imidazo[1,2-a]pyridin-10-ol; 3-(2-hydroxyethyl)-1-(5-((S)-4-phenyl-3,4-dihydro-1H-benzo[4,5]imidazo[2,1-c][1,4]oxazin-7-yl)pyrimidin-2-yl)pyrrolidin-3-ol; 4-(5-(9-(2-Methoxyphenyl)-6,7-dihydrobenzo[4,5]imidazo[1,2-a]pyridin-2-yl)pyrimidin-2-yl)morpholine; 10-(4-methoxyphenyl)-2-(2-morpholinopyrimidin-5-yl)-7,8,9,10-tetrahydro-6H-cyclohepta[4,5]imidazo[1,2-a]pyridin-10-ol; 7-(5-(1H-pyrazol-1-yl)pyrazin-2-yl)-4-phenyl-3,4-dihydro-1H-benzo[4,5]imidazo[2,1-c][1,4]oxazine; 1-phenyl-7-(pyridin-3-yl)-2,3-dihydro-1H-benzo[d]pyrrolo[1,2-a]imidazole; 5-(1-phenyl-2,3-dihydro-1H-benzo[d]pyrrolo[1,2-a]imidazol-7-yl)pyrimidin-2-amine; 2-(2-morpholinopyrimidin-5-yl)-10-(p-tolyl)-7,8,9,10-tetrahydro-6H-cyclohepta[4,5]imidazo[1,2-a]pyridin-10-ol; 5-(4-phenyl-3,4-dihydro-1H-benzo[4,5]imidazo[2,1-c][1,4]oxazin-7-yl)pyrimidine-2-carbonitrile; (R)-2-(5-(9-(2-methoxyphenyl)-6,7,8,9-tetrahydrobenzo[4,5]imidazo[1,2-a]pyridin-2-yl)pyrimidin-2-yl)propan-2-ol; (S)-7-(2-((R)-3-(methylsulfonyl)pyrrolidin-1-yl)pyrimidin-5-yl)-4-phenyl-3,4-dihydro-1H-benzo[4,5]imidazo[2,1-c][1,4]oxazine; 7-([1,2,5]oxadiazolo[3,4-b]pyridin-6-yl)-4-phenyl-3,4-dihydro-1H-benzo[4,5]imidazo[2,1-c][1,4]oxazine; 2-(5-(6-phenyl-7,8-dihydro-6H-pyrrolo[1,2′:1,2]imidazo[4,5-c]pyridin-3-yl)pyrimidin-2-yl)propan-2-ol; 7-(2-methylpyrimidin-5-yl)-4-phenyl-3,4-dihydro-1H-benzo[4,5]imidazo[2,1-c][1,4]oxazine; 1-phenyl-7-(pyrimidin-5-yl)-2,3-dihydro-1H-benzo[d]pyrrolo[1,2-a]imidazole; 7-(6-methoxy-5-methylpyridin-3-yl)-4-phenyl-3,4-dihydro-1H-benzo[4,5]imidazo[2,1-c][1,4]oxazine; 10-(4-chlorophenyl)-2-(2-morpholinopyrimidin-5-yl)-7,8,9,10-tetrahydro-6H-cyclohepta[4,5]imidazo[1,2-a]pyridin-10-ol; 2-(3-(1-phenyl-2,3-dihydro-1H-benzo[d]pyrrolo[1,2-a]imidazol-7-yl)phenyl)acetonitrile; N-(3-(1-phenyl-2,3-dihydro-1H-benzo[d]pyrrolo[1,2-a]imidazol-7-yl)benzyl)methanesulfonamide; 7-(6-methylpyridin-3-yl)-4-phenyl-3,4-dihydro-1H-benzo[4,5]imidazo[2,1-c][1,4]oxazine; (S)-2-(5-(1-phenyl-1,2,3,4-tetrahydrobenzo[4,5]imidazo[1,2-a]pyridin-8-yl)pyrimidin-2-yl)propan-2-ol; (S)-7-(2-(4-(1-methylpiperidin-4-yl)piperazin-1-yl)pyrimidin-5-yl)-4-phenyl-3,4-dihydro-1H-benzo[4,5]imidazo[2,1-c][1,4]oxazine; 4-phenyl-7-(6-(piperazin-1-yl)pyridin-3-yl)-3,4-dihydro-1H-benzo[4,5]imidazo[2,1-c][1,4]oxazine; (4S)-7-(2-(3-(methylsulfonyl)pyrrolidin-1-yl)pyrimidin-5-yl)-4-phenyl-3,4-dihydro-1H-benzo[4,5]imidazo[2,1-c][1,4]oxazine; (S)-8-(5-(4-phenyl-3,4-dihydro-1H-benzo[4,5]imidazo[2,1-c][1,4]oxazin-7-yl)pyrimidin-2-yl)-1,3,8-triazaspiro[4.5]decan-4-one; 7-(6-isopropoxy-5-methylpyridin-3-yl)-4-phenyl-3,4-dihydro-1H-benzo[4,5]imidazo[2,1-c][1,4]oxazine; 7-(5-methylpyridin-3-yl)-1-phenyl-2,3-dihydro-H-benzo[d]pyrrolo[1,2-a]imidazole; N-methyl-3-(1-phenyl-2,3-dihydro-1H-benzo[d]pyrrolo[1,2-a]imidazol-7-yl)benzamide; 4-(1-phenyl-2,3-dihydro-1H-benzo[d]pyrrolo[1,2-a]imidazol-7-yl)-N-((tetrahydrofuran-2-yl)methyl)benzamide; (S)-4-(5-(1-phenyl-2,3-dihydro-1H-benzo[d]pyrrolo[1,2-a]imidazol-7-yl)pyrimidin-2-yl)morpholine; N-(5-(4-phenyl-3,4-dihydro-1H-benzo[4,5]imidazo[2,1-c][1,4]oxazin-7-yl)pyridin-2-yl)acetamide; N-ethyl-N-methyl-3-(1-phenyl-2,3-dihydro-1H-benzo[d]pyrrolo[1,2-a]imidazol-7-yl)benzamide; 5-(1-phenyl-2,3-dihydro-1H-benzo[d]pyrrolo[1,2-a]imidazol-7-yl)pyridin-2-amine; 7-(3-methyl-3H-imidazo[4,5-b]pyridin-6-yl)-4-phenyl-3,4-dihydro-1H-benzo[4,5]imidazo[2,1-c][1,4]oxazine; 10-(3,5-dimethoxyphenyl)-2-(2-morpholinopyrimidin-5-yl)-7,8,9,10-tetrahydro-6H-cyclohepta[4,5]imidazo[1,2-a]pyridin-10-ol; N,N-dimethyl-3-((5-(4-phenyl-3,4-dihydro-1H-benzo[4,5]imidazo[2,1-c][1,4]oxazin-7-yl)pyridin-2-yl)oxy)propan-1-amine; (3R,4R)-1-(5-((S) 4-phenyl-3,4-dihydro-1H-benzo[4,5]imidazo[2,1-c][1,4]oxazin-7-yl)pyrimidin-2-yl)pyrrolidine-3,4-diol; N-(2-(dimethylamino)ethyl)-3-(1-phenyl-2,3-dihydro-1H-benzo[d]pyrrolo[1,2-a]imidazol-7-yl)benzamide; 10-(3-methoxyphenyl)-2-(2-morpholinopyrimidin-5-yl)-7,8,9,10-tetrahydro-6H-cyclohepta[4,5]imidazo[1,2-a]pyridin-10-ol; 7-(1,5-dimethyl-1H-pyrazol-4-yl)-1-phenyl-2,3-dihydro-1H-benzo[d]pyrrolo[1,2-a]imidazole; N,N-dimethyl-3-(1-phenyl-2,3-dihydro-1H-benzo[d]pyrrolo[1,2-a]imidazol-7-yl)benzamide; 7-(3-(methylsulfonyl)phenyl)-1-phenyl-2,3-dihydro-1H-benzo[d]pyrrolo[1,2-a]imidazole; 4-phenyl-7-(pyrido[2,3-b]pyrazin-7-yl)-3,4-dihydro-1H-benzo[4,5]imidazo[2,1-c][1,4]oxazine; 1-methyl-5-(1-phenyl-2,3-dihydro-1H-benzo[d]pyrrolo[1,2-a]imidazol-7-yl)pyridin-2(1H)-one; (3S,4S)-1-(5-((S)-4-phenyl-3,4-dihydro-1H-benzo[4,5]imidazo[2,1-c][1,4]oxazin-7-yl)pyrimidin-2-yl)pyrrolidine-3,4-diol; 4-phenyl-7-(pyrimidin-5-yl)-3,4-dihydro-1H-benzo[4,5]imidazo[2,1-c][1,4]oxazine; (S)-2-(5-(1-(2-Methoxyphenyl)-2,3-dihydro-1H-cyclopenta[4,5]imidazo[1,2-a]pyridin-7-yl)pyrimidin-2-yl)propan-2-ol; (S)-2-(5-(1-phenyl-2,3-dihydro-1H-benzo[d]pyrrolo[1,2-a]imidazol-7-yl)pyrimidin-2-yl)propan-2-ol; 2-(2-(2-(2-hydroxypropan-2-yl)pyrimidin-5-yl)-6,7,8,9-tetrahydrobenzo[4,5]imidazo[1,2-a]pyridin-9-yl)phenol; 4-(5-(6-phenyl-6,7,8,9-tetrahydroimidazo[1,2-a:4,5-b′]dipyridin-3-yl)pyrimidin-2-yl)piperazin-2-one; (S)-7-(2-(3-morpholinoazetidin-1-yl)pyrimidin-5-yl)-4-phenyl-3,4-dihydro-1H-benzo[4,5]imidazo[2,1-c][1,4]oxazine; 7-(5-(methylsulfonyl)pyridin-3-yl)-1-phenyl-2,3-dihydro-1H-benzo[d]pyrrolo[1,2-a]imidazole; (R)-7-(2-Morpholinopyrimidin-5-yl)-4-phenyl-3,4-dihydro-1H-benzo[4,5]imidazo[2,1-c][1,4]oxazine; 7-(2-methylpyridin-3-yl)-1-phenyl-2,3-dihydro-1H-benzo[d]pyrrolo[1,2-a]imidazole; 1-(5-(1-phenyl-1,2,3,4-tetrahydrobenzo[4,5]imidazo[1,2-a]pyridin-8-yl)pyrimidin-2-yl)azetidine-3-carboxylic acid; 7-(1-methyl-1H-pyrrol-3-yl)-1-phenyl-2,3-dihydro-1H-benzo[d]pyrrolo[1,2-a]imidazole; or N-(2-morpholinoethyl)-5-(4-phenyl-3,4-dihydro-1H-benzo[4,5]imidazo[2,1-c][1,4]oxazin-7-yl)pyridin-2-amine. In certain embodiments, the compound is selected from the group consisting of: 7-(5-((R)-1-Phenyl-2,3-dihydro-1H-benzo[d]pyrrolo[1,2-a]imidazol-7-yl)pyrimidin-2-yl)hexahydroimidazo[1,5-a]pyrazin-3(2H)-one; 3,3-difluoro-1-(5-((R)-1-phenyl-2,3-dihydro-1H-benzo[d]pyrrolo[1,2-a]imidazol-7-yl)pyrimidin-2-yl)piperidin-4-ol; (S)-2-(5-(4-(2-(Difluoromethoxy)phenyl)-3,4-dihydro-1H-benzo[4,5]imidazo[2,1-c][1,4]oxazin-7-yl)pyrimidin-2-yl)propan-2-ol; (S)-2-(5-(9-(2-methoxyphenyl)-6,7,8,9-tetrahydrobenzo[4,5]imidazo[1,2-a]pyridin-2-yl)pyrimidin-2-yl)propan-2-ol; (R)-7-(5-(1-phenyl-2,3-dihydro-1H-benzo[d]pyrrolo[1,2-a]imidazol-7-yl)pyrimidin-2-yl)-7-azaspiro[3.5]nonan-2-ol; 1-(5-(8-(2,5-dimethylphenyl)-7,8-dihydro-6H-pyrrolo[2′,1′:2,3]imidazo[4,5-b]pyridin-2-yl)pyrimidin-2-yl)piperidin-4-ol; 4-(5-(10-(2-methoxyphenyl)-7,8,9,10-tetrahydro-6H-cyclohepta[4,5]imidazo[1,2-a]pyridin-2-yl)pyrimidin-2-yl)morpholine; (R)-4-(5-(1-phenyl-2,3-dihydro-1H-benzo[d]pyrrolo[1,2-a]imidazol-7-yl)pyrimidin-2-yl)morpholine; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one; 7-(5-((S)-4-phenyl-3,4-dihydro-1H-benzo[4,5]imidazo[2,1-c][1,4]oxazin-7-yl)pyrimidin-2-yl)hexahydroimidazo[1,5-a]pyrazin-3(2H)-one; 4-(5-(8-(2,5-dimethylphenyl)-7,8-dihydro-6H-pyrrolo[2′,1′:2,3]imidazo[4,5-b]pyridin-2-yl)pyrimidin-2-yl)piperazin-2-one; 2-(5-(1-(2,5-dimethylphenyl)-2,3-dihydro-1H-benzo[d]pyrrolo[1,2-a]imidazol-7-yl)pyrimidin-2-yl)propan-2-ol; 3,3-difluoro-1-(5-((S)-4-phenyl-3,4-dihydro-1H-benzo[4,5]imidazo[2,1-c][1,4]oxazin-7-yl)pyrimidin-2-yl)piperidin-4-ol; 7-(5-(4-(3-fluorophenyl)-3,4-dihydro-1H-benzo[4,5]imidazo[2,1-c][1,4]oxazin-7-yl)pyrimidin-2-yl)hexahydroimidazo[1,5-a]pyrazin-3(2H)-one; (R)-2-(5-(1-(2-methoxyphenyl)-2,3-dihydro-1H-cyclopenta[4,5]imidazo[1,2-a]pyridin-7-yl)pyrimidin-2-yl)propan-2-ol; 7-(4-(isopropylsulfonyl)phenyl)-1-phenyl-2,3-dihydro-1H-benzo[d]pyrrolo[1,2-a]imidazole; 2-(5-(9-(2-Methoxyphenyl)-6,7,8,9-tetrahydrobenzo[4,5]imidazo[1,2-a]pyridin-2-yl)pyrimidin-2-yl)propan-2-ol; 4-(5-(8-(2,5-dimethylphenyl)-7,8-dihydro-6H-pyrrolo[2′,1′:2,3]imidazo[4,5-b]pyridin-2-yl)pyrimidin-2-yl)morpholine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 4-(5-(9-(2-methoxyphenyl)-6,7,8,9-tetrahydrobenzo[4,5]imidazo[1,2-a]pyridin-2-yl)pyrimidin-2-yl)morpholine; 1-(5-(9-phenyl-6,7,8,9-tetrahydroimidazo[1,2-a:5,4-b′]dipyridin-2-yl)pyrimidin-2-yl)piperidin-4-ol; (R)-2-(5-(1-Phenyl-2,3-dihydro-1H-benzo[d]pyrrolo[1,2-a]imidazol-7-yl)pyrimidin-2-yl)propan-2-ol; 2-(5-(10-(2-methoxyphenyl)-7,8,9,10-tetrahydro-6H-cyclohepta[4,5]imidazo[1,2-a]pyridin-2-yl)pyrimidin-2-yl)propan-2-ol; N-methyl-4-(1-phenyl-2,3-dihydro-1H-benzo[d]pyrrolo[1,2-a]imidazol-7-yl)benzenesulfonamide; (S)-7-(2-morpholinopyrimidin-5-yl)-4-phenyl-3,4-dihydro-1H-benzo[4,5]imidazo[2,1-c][1,4]oxazine; (S)-7-(5-(4-phenyl-3,4-dihydro-1H-benzo[4,5]imidazo[2,1-c][1,4]oxazin-7-yl)pyrimidin-2-yl)-7-azaspiro[3.5]nonan-2-ol; 2-(5-(1-(2-methoxyphenyl)-2,3-dihydro-1H-cyclopenta[4,5]imidazo[1,2-a]pyridin-7-yl)pyrimidin-2-yl)propan-2-ol; (R)-2-(5-(1-phenyl-1,2,3,4-tetrahydrobenzo[4,5]imidazo[1,2-a]pyridin-8-yl)pyrimidin-2-yl)propan-2-ol; ethyl 2-[[5-[9-(2-methoxyphenyl)-6,7,8,9-tetrahydropyrido[1,2-a]benzimidazol-2-yl]pyrimidin-2-yl]amino]acetate.r 2-((5-(9-(2-methoxyphenyl)-6,7,8,9-tetrahydrobenzo[4,5]imidazo[1,2-a]pyridin-2-yl)pyridin-2-yl)oxy)acetic acid. In certain embodiments, the compound is selected from the group consisting of: (8aR)-7-(5-(6′,8′-dihydro-2H-spiro[benzofuran-3,9′-pyrido[3′,2′:4,5]imidazo[2,1-c][1,4]oxazin]-2′-yl)pyrimidin-2-yl)hexahydroimidazo[1,5-a]pyrazin-3(2H)-one; 3-((5-(6′,8′-dihydro-2H-spiro[benzofuran-3,9′-pyrido[3′,2′:4,5]imidazo[2,1-c][1,4]oxazin]-2′-yl)pyrimidin-2-yl)amino)cyclobutanol; 5-(6′,8′-dihydro-2H-spiro[benzofuran-3,9′-pyrido[3′,2′:4,5]imidazo[2,1-c][1,4]oxazin]-2′-yl)-N-(tetrahydrofuran-3-yl)pyrimidin-2-amine; 1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-3-ol; 2′-(2-(4-methylpiperazin-1-yl)pyrimidin-5-yl)-6′,8′-dihydro-2H-spiro[benzofuran-3,9′-pyrido[3′,2′:4,5]imidazo[2,1-c][1,4]oxazine]; 2′-(2-morpholinopyrimidin-5-yl)-6′,8′-dihydro-2H-spiro[benzofuran-3,9′-pyrido[3′,2′:4,5]imidazo[2,1-c][1,4]oxazine]; 1-(5-(1,2′,3,3′-tetrahydrospiro[benzo[4,5]imidazo[2,1-c][1,4]oxazine-4,1′-inden]-7-yl)pyrimidin-2-yl)piperidin-4-ol; (1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-3-yl)methanol; (8aS)-7-(5-(2,3,6′,8′-tetrahydrospiro[indene-1,9′-pyrido[3′,2′:4,5]imidazo[2,1-c][1,4]oxazin]-2′-yl)pyrimidin-2-yl)hexahydroimidazo[1,5-a]pyrazin-3(2H)-one; 2′-(2-(1,4-oxazepan-4-yl)pyrimidin-5-yl)-2,3,6′,8′-tetrahydrospiro[indene-1,9′-pyrido[3′,2′:4,5]imidazo[2,1-c][1,4]oxazine]; 3,3-difluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-ol; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)propan-1-one; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)ethanone; (4-fluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-yl)methanol; 2′-(2-morpholinopyrimidin-5-yl)-2,3,6′,8′-tetrahydrospiro[indene-1,9′-pyrido[3′,2′:4,5]imidazo[2,1-c][1,4]oxazine]; 1-(5-(2,3,6′,8′-tetrahydrospiro[indene-1,9′-pyrido[3′,2′:4,5]imidazo[2,1-c][1,4]oxazin]-2′-yl)pyrimidin-2-yl)azepan-4-ol; (S)-2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol; (R)-2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol; (8aR)-7-(5-(6′,8′-dihydro-2H-spiro[benzofuran-3,9′-pyrido[3′,2′:4,5]imidazo[2,1-c][1,4]oxazin]-2′-yl)pyrimidin-2-yl)hexahydroimidazo[1,5-a]pyrazin-3(2H)-one; 3-((5-(6′,8′-dihydro-2H-spiro[benzofuran-3,9′-pyrido[3′,2′:4,5]imidazo[2,1-c][1,4]oxazin]-2′-yl)pyrimidin-2-yl)amino)cyclobutanol; 5-(6′,8′-dihydro-2H-spiro[benzofuran-3,9′-pyrido[3′,2′:4,5]imidazo[2,1-c][1,4]oxazin]-2′-yl)-N-(tetrahydrofuran-3-yl)pyrimidin-2-amine; 1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-3-ol; 2′-(2-(4-methylpiperazin-1-yl)pyrimidin-5-yl)-6′,8′-dihydro-2H-spiro[benzofuran-3,9′-pyrido[3′,2′:4,5]imidazo[2,1-c][1,4]oxazine]; 2′-(2-morpholinopyrimidin-5-yl)-6′,8′-dihydro-2H-spiro[benzofuran-3,9′-pyrido[3′,2′:4,5]imidazo[2,1-c][1,4]oxazine]; 1-(5-(1,2′,3,3′-tetrahydrospiro[benzo[4,5]imidazo[2,1-c][1,4]oxazine-4,1′-inden]-7-yl)pyrimidin-2-yl)piperidin-4-ol; (1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-3-yl)methanol; (8aR) 7-(5-(2,3,6′,8′-tetrahydrospiro[indene-1,9′-pyrido[3′,2′:4,5]imidazo[2,1-c][1,4]oxazin]-2′-yl)pyrimidin-2-yl)hexahydroimidazo[1,5-a]pyrazin-3(2H)-one; 2′-(2-(1,4-oxazepan-4-yl)pyrimidin-5-yl)-2,3,6′,8′-tetrahydrospiro[indene-1,9′-pyrido[3′,2′:4,5]imidazo[2,1-c][1,4]oxazine]; 3,3-difluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-ol; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)propan-1-one; 2-hydroxy-1-(4-(5-(2,3,6′,8′-tetrahydrospiro[indene-1,9′-pyrido[3′,2′:4,5]imidazo[2,1-c][1,4]oxazin]-2′-yl)pyrimidin-2-yl)piperazin-1-yl)ethanone; (4-fluoro-1-(5-(2,3,6′,8′-tetrahydrospiro[indene-1,9′-pyrido[3′,2′:4,5]imidazo[2,1-c][1,4]oxazin]-2′-yl)pyrimidin-2-yl)piperidin-4-yl)methanol; 2′-(2-morpholinopyrimidin-5-yl)-2,3,6′,8′-tetrahydrospiro[indene-1,9′-pyrido[3′,2′:4,5]imidazo[2,1-c][1,4]oxazine]; 1-(5-(2,3,6′,8′-tetrahydrospiro[indene-1,9′-pyrido[3′,2′:4,5]imidazo[2,1-c][1,4]oxazin]-2′-yl)pyrimidin-2-yl)azepan-4-ol; (R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2(1H)-one; (1r,4r) 4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol; (1s,4s)-4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol; 3-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclopentanol; 2′-(2-morpholinopyrimidin-5-yl)-6′,7′-dihydrospiro[cyclohexane-1,9′-pyrano[4′,3′:4,5]imidazo[1,2-b]pyridazine]; 2′-(2-morpholinopyrimidin-5-yl)-6′,7′-dihydrospiro[chroman-4,9′-pyrano[4′,3′:4,5]imidazo[1,2-b]pyridazine]; 2′-(2-(piperazin-1-yl)pyrimidin-5-yl)-6′H,8′H-spiro[chromane-4,9′-pyrido[3′,2′:4,5]imidazo[2,1-c][1,4]oxazine]; 2′-(2-methoxypyrimidin-5-yl)-6′,8′-dihydrospiro[chroman-4,9′-pyrido[3,2′:4,5]imidazo[2,1-c][1,4]oxazine]; 2′-(2-ethoxypyrimidin-5-yl)-6′,8-dihydrospiro[chroman-4,9′-pyrido[3,2′:4,5]imidazo[2,1-c][1,4]oxazine]; 2′-(2-(methylsulfonyl)pyrimidin-5-yl)-6′,8′-dihydrospiro[chroman-4,9′-pyrido[3′,2′:4,5]imidazo[2,1-c][1,4]oxazine]; 2′-(2-(1,4-diazepan-1-yl)pyrimidin-5-yl)-6′,8′-dihydrospiro[chroman-4,9′-pyrido[3′,2′:4,5]imidazo[2,1-c][1,4]oxazine]; 5-(6′,8′-dihydrospiro[chroman-4,9′-pyrido[3′,2′:4,5]imidazo[2,1-c][1,4]oxazin]-2′-yl)-N-isopropylpyrimidin-2-amine; 2′-(2-morpholinopyrimidin-5-yl)-6′,8-dihydrospiro[chroman-4,9′-pyrido[3′,2′:4,5]imidazo[2,1-c][1,4]oxazine]; 2-(5-(6′,8′-dihydrospiro[chroman-4,9′-pyrido[3′,2′:4,5]imidazo[2,1-c][1,4]oxazin]-2′-yl)pyrimidin-2-yl)propan-2-ol; 2′-(2-((tetrahydro-2H-pyran-4-yl)oxy)pyridin-4-yl)-6′,8′-dihydrospiro[chroman-4,9′-pyrido[3′,2′:4,5]imidazo[2,1-c][1,4]oxazine]; 5-(6′H,8′H-Spiro[chromane-4,9′-pyrido[3′,2′:4,5]imidazo[2,1-c][1,4]oxazin]-2′-yl)-N-(tetrahydrofuran-3-yl)pyrimidin-2-amine; (8aR)-7-(5-(6′,8′-dihydrospiro[chroman-4,9′-pyrido[3′,2′:4,5]imidazo[2,1-c][1,4]oxazin]-2′-yl)pyrimidin-2-yl)hexahydroimidazo[1,5-a]pyrazin-3(2H)-one; 1-(5-(6′,8′-dihydrospiro[chroman-4,9′-pyrido[3′,2′:4,5]imidazo[2,1-c][1,4]oxazin]-2′-yl)pyrimidin-2-yl)piperidin-4-ol; 1-(5-(6′,8′-dihydrospiro[chroman-4,9′-pyrido[3′,2′:4,5]imidazo[2,1-c][1,4]oxazin]-2′-yl)pyrimidin-2-yl)piperidin-3-ol; 1-(5-(6′,8′-dihydrospiro[chroman-4,9′-pyrido[3′,2′:4,5]imidazo[2,1-c][1,4]oxazin]-2′-yl)pyrimidin-2-yl)azetidin-3-ol; 1-(5-(6′,8′-dihydrospiro[chroman-4,9′-pyrido[3′,2′:4,5]imidazo[2,1-c][1,4]oxazin]-2′-yl)pyrimidin-2-yl)pyrrolidin-3-ol; 3-((5-(6′,8′-dihydrospiro[chroman-4,9′-pyrido[3′,2′:4,5]imidazo[2,1-c][1,4]oxazin]-2′-yl)pyrimidin-2-yl)amino)cyclobutanol; 1-(5-(6′,8′-dihydrospiro[chroman-4,9′-pyrido[3′,2′:4,5]imidazo[2,1-c][1,4]oxazin]-2′-yl)pyrimidin-2-yl)-3-methylazetidin-3-ol; 2′-(2-(4-methylpiperazin-1-yl)pyrimidin-5-yl)-6′,8′-dihydrospiro[chroman-4,9′-pyrido[3′,2′:4,5]imidazo[2,1-c][1,4]oxazine]; 2′-(5,5-Dimethyl-2,5-dihydro-1H-pyrrol-3-yl) 2H,6′H,8′H-spiro[benzofuran-3,9′-pyrido[3′,2′:4,5]imidazo[2,1-c][1,4]oxazine]; 2′-(2,5-dihydro-1H-pyrrol-3-yl)-6′,8′-dihydro-2H-spiro[benzofuran-3,9′-pyrido[3′,2′:4,5]imidazo[2,1-c][1,4]oxazine]; 2′-(2-(piperazin-1-yl)pyrimidin-5-yl)-2H,6′H,8′H-spiro[benzofuran-3,9′-pyrido[3′,2′:4,5]imidazo[2,1-c][1,4]oxazine]; 2′-(2-((tetrahydro-2H-pyran-4-yl)oxy)pyridin-4-yl) 6′,8′-dihydro-2H-spiro[benzofuran-3,9′-pyrido[3′,2′:4,5]imidazo[2,1-c][1,4]oxazine]; 2′-(2-methoxypyrimidin-5-yl)-6′,8′-dihydro-2H-spiro[benzofuran-3,9′-pyrido[3′,2′:4,5]imidazo[2,1-c][1,4]oxazine]; 2-(tert-butoxy)-1-((2S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)ethanone; 2-(tert-butoxy)-1-((3S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)ethanone; 2-(tert-butoxy)-1-((3R)-4-(5-(6′,8′-dihydro-2H-spiro[benzofuran-3, 9′-pyrido[3′,2′:4,5]imidazo[2,1-c][1,4]oxazin]-2′-yl)pyrimidin-2-yl)-3-methylpiperazin-1-yl)ethanone; 2-(tert-butoxy)-1-((2R)-4-(5-(6′,8′-dihydro-2H-spiro[benzofuran-3, 9′-pyrido[3′,2′:4,5]imidazo[2,1-c][1,4]oxazin]-2′-yl)pyrimidin-2-yl)-2-methylpiperazin-1-yl)ethanone; 1-((2S)-4-(5-(2H,6′H,8′H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethan-1-one 1-((3S)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)-2-hydroxyethanone; 1-((3R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-3-methylpiperazin-1-yl)-2-hydroxyethanone; 1-((2R)-4-(5-(6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 2-(5-(2,3-dihydro-6′H,8′H-spiro[indene-1,9′-pyrido[3′,2′:4,5]imidazo[2,1-c][1,4]oxazin]-2′-yl)pyrimidin-2-yl)propan-2-ol; 2′-(2-(2-hydroxypropan-2-yl)pyrimidin-5-yl) 6′,8′-dihydrospiro[indene-1,9′-pyrido[3′,2′:4,5]imidazo[2,1-c][1,4]oxazin]-3(2H)-one; 2′-(2-(2-hydroxypropan-2-yl)pyrimidin-5-yl)-2,3-dihydro-6′H,8′H-spiro[indene-1,9′-pyrido[3′,2′:4,5]imidazo[2,1-c][1,4]oxazin]-6′-ol; 2′-(1-(pyrimidin-4-yl)-1,2,3,6-tetrahydropyridin-4-yl)-2H,6′H,8′H-spiro[benzofuran-3,9′-pyrido[3′,2′:4,5]imidazo[2,1-c][1,4]oxazine]; 1-(4-(6′,8′-dihydro-2H-spiro[benzofuran-3,9′-pyrido[3′,2′:4,5]imidazo[2,1-c][1,4]oxazin]-2′-yl)-5,6-dihydropyridin-1(2H)-yl)-3-methoxy-3-methylbutan-1-one; (S)-1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-4-ol; (R)-7-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)hexahydroimidazo[1,5-a]pyrazin-3(2H)-one; (1S,4r)-4-((4-((S)-6′,8′-Dihydro-2H-spiro[benzofuran-3, 9′-pyrido[3′,2′:4,5]imidazo[2,1-c][1,4]oxazin]-2′-yl)pyridin-2-yl)oxy)cyclohexanol; 1-((S)-4-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-((R)-4-(5-((S)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-((R)-4-(5-((R)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-((S)-4-(5-((R)-6′,8′-dihydro-2H-spiro[benzofuran-3,9′-pyrido[3′,2′:4,5]imidazo[2,1-c][1,4]oxazin]-2′-yl)pyrimidin-2-yl)-2-methylpiperazin-1-yl)-2-hydroxyethanone; 1-(5-(6′,8′-dihydrospiro[isochroman-4,9′-pyrido[3′,2′:4,5]imidazo[2,1-c][1,4]oxazin]-2′-yl)pyrimidin-2-yl)piperidin-4-ol; (8aR)-7-(5-(6′,8′-dihydrospiro[isochroman-4,9′-pyrido[3′,2′:4,5]imidazo[2,1-c][1,4]oxazin]-2′-yl)pyrimidin-2-yl)hexahydroimidazo[1,5-a]pyrazin-3(2H)-one; 2′-(2-morpholinopyrimidin-5-yl)-6′,8′-dihydrospiro[isochroman-4,9′-pyrido[3′,2′:4,5]imidazo[2,1-c][1,4]oxazine]; 2-(5-(2,3-dihydro-6′H,8′H-spiro[indene-1,9′-pyrido[3′,2′:4,5]imidazo[2,1-c][1,4]oxazin]-2′-yl)pyrimidin-2-yl)propan-2-amine; (S)-2-(5-(6′,8′-dihydro-2H-spiro[benzofuran-3,9′-pyrido[3′,2′:4,5]imidazo[2,1-c][1,4]oxazin]-2′-yl)pyrimidin-2-yl)propan-2-amine; or 2′-(2-(2-hydroxypropan-2-yl)pyrimidin-5-yl)-2,3,6′,8′-tetrahydrospiro[indene-1,9′-pyrido[3′,2′:4,5]imidazo[2,1-c][1,4]oxazin]-3-ol. In certain embodiments, the compound is selected from the group consisting of: ((R)-1-(5-((R)-8-phenyl-7,8-dihydro-6H-pyrrolo[2′,1′:2,3]imidazo[4,5-b]pyridin-2-yl)pyrimidin-2-yl)pyrrolidin-2-yl)methanol; ((S)-1-(5-((R)-8-phenyl-7,8-dihydro-6H-pyrrolo[2′,1′:2,3]imidazo[4,5-b]pyridin-2-yl)pyrimidin-2-yl)pyrrolidin-2-yl)methanol; (R)-1-(2-(methylsulfonyl)ethyl)-4-(8-phenyl-7,8-dihydro-6H-pyrrolo[2′,1′:2,3]imidazo[4,5-b]pyridin-2-yl)pyridin-2(1H)-one; (R)-1-(2-hydroxy-2-methylpropyl)-4-(8-phenyl-7,8-dihydro-6H-pyrrolo[2′,1′:2,3]imidazo[4,5-b]pyridin-2-yl)pyridin-2(1H)-one; 1-(5-((R)-8-phenyl-7,8-dihydro-6H-pyrrolo[2′,1′:2,3]imidazo[4,5-b]pyridin-2-yl)pyrimidin-2-yl)ethanol; 2-cyclopropyl-1-(5-((R)-8-phenyl-7,8-dihydro-6H-pyrrolo[2′,1′:2,3]imidazo[4,5-b]pyridin-2-yl)pyrimidin-2-yl)ethanol; (5-((R)-8-phenyl-7,8-dihydro-6H-pyrrolo[2′,1′:2,3]imidazo[4,5-b]pyridin-2-yl)pyrimidin-2-yl)(tetrahydro-2H-pyran-4-yl)methanol; 1-(5-((R)-8-phenyl-7,8-dihydro-6H-pyrrolo[2′,1′:2,3]imidazo[4,5-b]pyridin-2-yl)pyrimidin-2-yl)-1-(tetrahydro-2H-pyran-4-yl)ethanol; 1-cyclopropyl-2-(5-((R)-8-phenyl-7,8-dihydro-6H-pyrrolo[2′,1′:2,3]imidazo[4,5-b]pyridin-2-yl)pyrimidin-2-yl)propan-2-ol; 1-((R)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; 1-((S)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; (1R,3R)-3-((4-((R)-8-phenyl-7,8-dihydro-6H-pyrido[3,2-b]pyrrolizin-2-yl)pyridin-2-yl)oxy)cyclopentanecarbonitrile; (R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2(1H)-one; 2-(5-(8-(pyridin-2-yl)-7,8-dihydro-6H-pyrrolo[2′,1′:2,3]imidazo[4,5-b]pyridin-2-yl)pyrimidin-2-yl)propan-2-ol; 1-((S)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; 2-hydroxy-1-((R)-2-methyl-4-(5-((R)-8-phenyl-7,8-dihydro-6H-pyrrolo[2′,1′:2,3]imidazo[4,5-b]pyridin-2-yl)pyrimidin-2-yl)piperazin-1-yl)ethanone; (R)-7-(5-((R)-8-(3-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; 1-((R)-4-(5-((R)-8-(3-methoxyphenyl)-7,8-dihydro-6H-pyrrolo[2′,1′:2,3]imidazo[4,5-b]pyridin-2-yl)pyrimidin-2-yl)-2-methylpiperazin-1-yl)ethanone; (R)-7-(5-((S)-8-(3-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; (R)-7-(5-((R)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; (S)-7-(5-((R)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; (R)-1-(5-(8-(2-methoxyphenyl)-7,8-dihydro-6H-pyrrolo[2′,1′:2,3]imidazo[4,5-b]pyridin-2-yl)pyrimidin-2-yl)piperidin-4-ol; 2-hydroxy-1-((R)-4-(5-((R)-8-(2-methoxyphenyl)-7,8-dihydro-6H-pyrrolo[2′,1′:2,3]imidazo[4,5-b]pyridin-2-yl)pyrimidin-2-yl)-2-methylpiperazin-1-yl)ethanone; (R)-7-(5-((S)-8-(2-methoxyphenyl)-7,8-dihydro-6H-pyrrolo[2′,1′:2,3]imidazo[4,5-b]pyridin-2-yl)pyrimidin-2-yl)hexahydroimidazo[1,5-a]pyrazin-3(2H)-one; 1-((R)-4-(5-((R)-8-(3-(Hydroxymethyl)phenyl)-7,8-dihydro-6H-pyrrolo[2′,1′:2,3]imidazo[4,5-b]pyridin-2-yl)pyrimidin-2-yl)-2-methylpiperazin-1-yl)ethanone; 1-((R)-4-(5-((R)-8-(3-(Hydroxymethyl)phenyl)-7,8-dihydro-6H-pyrrolo[2′,1′:2,3]imidazo[4,5-b]pyridin-2-yl)pyrimidin-2-yl)-2-methylpiperazin-1-yl) ethanone; (S)-1-(5-((R)-8-phenyl-7,8-dihydro-6H-pyrrolo[2′,1′:2,3]imidazo[4,5-b]pyridin-2-yl)pyrimidin-2-yl)piperidin-4-yl 2-amino-3-methylbutanoate; (R)-1-(5-(8-phenyl-7,8-dihydro-6H-pyrrolo[2′,1′:2,3]imidazo[4,5-b]pyridin-2-yl)pyrimidin-2-yl)piperidin-4-yl dihydrogen phosphate hydrochloride; (R)-8-phenyl-2-(2-((tetrahydro-2H-pyran-4-yl)oxy)pyridin-4-yl)-7,8-dihydro-6H-pyrrolo[2′,1′:2,3]imidazo[4,5-b]pyridine; 3-((4-((R)-8-phenyl-7,8-dihydro-6H-pyrrolo[2′,1:2,3]imidazo[4,5-b]pyridin-2-yl)pyridin-2-yl)oxy)cyclopentanol; (R)-4-((4-(8-phenyl-7,8-dihydro-6H-pyrrolo[2′,1:2,3]imidazo[4,5-b]pyridin-2-yl)pyridin-2-yl)oxy)cyclohexanol; (R)-2-(2-(oxetan-3-yloxy)pyridin-4-yl)-8-phenyl-7,8-dihydro-6H-pyrrolo[2′,1′:2,3]imidazo[4,5-b]pyridine; 3-((4-((R)-8-phenyl-7,8-dihydro-6H-pyrrolo[2′,1:2,3]imidazo[4,5-b]pyridin-2-yl)pyridin-2-yl)oxy)cyclohexanol; (R)-2-(2-(oxetan-3-ylmethoxy)pyridin-4-yl)-8-phenyl-7,8-dihydro-6H-pyrrolo[2′,1′:2,3]imidazo[4,5-b]pyridine; (R)-2-(2-(((R)-1-methylpyrrolidin-3-yl)oxy)pyridin-4-yl)-8-phenyl-7,8-dihydro-6H-pyrrolo[2′,1′:2,3]imidazo[4,5-b]pyridine; (R)-2-((4-(8-phenyl-7,8-dihydro-6H-pyrrolo[2′,1:2,3]imidazo[4,5-b]pyridin-2-yl)pyridin-2-yl)oxy)ethanol; (R)-2-(2-(((S)-1-methylpyrrolidin-3-yl)oxy)pyridin-4-yl)-8-phenyl-7,8-dihydro-6H-pyrrolo[2′,1′:2,3]imidazo[4,5-b]pyridine; (1S,4s)-4-((4-((R)-8-phenyl-7,8-dihydro-6H-pyrrolo[2′,1′:2,3]imidazo[4,5-b]pyridin-2-yl)pyridin-2-yl)oxy)cyclohexanol; (8R)-8-phenyl-2-(2-((tetrahydro-2H-pyran-3-yl)oxy)pyridin-4-yl)-7,8-dihydro-6H-pyrrolo[2′,1′:2,3]imidazo[4,5-b]pyridine; (8R)-8-phenyl-2-(2-((tetrahydrofuran-3-yl)oxy)pyridin-4-yl)-7,8-dihydro-6H-pyrrolo[2′,1′:2,3]imidazo[4,5-b]pyridine; (R)-2-(2-(cyclopentyloxy)pyridin-4-yl)-8-phenyl-7,8-dihydro-6H-pyrrolo[2′,1′:2,3]imidazo[4,5-b]pyridine; (R)-2-(2-(cyclohexyloxy)pyridin-4-yl)-8-phenyl-7,8-dihydro-6H-pyrrolo[2′,1′:2,3]imidazo[4,5-b]pyridine; (R)-methyl 4-((4-(8-phenyl-7,8-dihydro-6H-pyrrolo[2′,1′:2,3]imidazo[4,5-b]pyridin-2-yl)pyridin-2-yl)oxy)cyclohexanecarboxylate; methyl 3-((4-((R)-8-phenyl-7,8-dihydro-6H-pyrrolo[2′,1′:2,3]imidazo[4,5-b]pyridin-2-yl)pyridin-2-yl)oxy)cyclopentanecarboxylate; (R)-2-(2-butoxypyridin-4-yl)-8-phenyl-7,8-dihydro-6H-pyrido[3,2-b]pyrrolizine; (1R,3R)-3-((4-((R)-8-phenyl-7,8-dihydro-6H-pyrido[3,2-b]pyrrolizin-2-yl)pyridin-2-yl)oxy)cyclopentanecarbonitrile; (R)-8-phenyl-2-(2-((S)-pyrrolidin-3-yloxy)pyridin-4-yl)-7,8-dihydro-6H-pyrrolo[2′,1′:2,3]imidazo[4,5-b]pyridine; (8R)-8-phenyl-2-(2-(piperidin-3-yloxy)pyridin-4-yl) 7,8-dihydro-6H-pyrrolo[2′,1′:2,3]imidazo[4,5-b]pyridine; (R)-8-phenyl-2-(2-((R)-pyrrolidin-3-yloxy)pyridin-4-yl)-7,8-dihydro-6H-pyrrolo[2′,1′:2,3]imidazo[4,5-b]pyridine; (8R)-2-(2-(6-azaspiro[3.4]octan-1-yloxy)pyridin-4-yl)-8-phenyl-7,8-dihydro-6H-pyrrolo[2′,1′:2,3]imidazo[4,5-b]pyridine; (R)-2-(2-(6-azaspiro[3.4]octan-2-yloxy)pyridin-4-yl)-8-phenyl-7,8-dihydro-6H-pyrrolo[2′,1′:2,3]imidazo[4,5-b]pyridine; (8R)-2-(2-(6-azaspiro[3.5]nonan-1-yloxy)pyridin-4-yl)-8-phenyl-7,8-dihydro-6H-pyrrolo[2′,1′:2,3]imidazo[4,5-b]pyridine; 1-(5-(6′,8′-dihydro-2H-spiro[benzofuran-3,9′-pyrido[3′,2′:4,5]imidazo[2,1-c][1,4]oxazin]-2′-yl)pyrimidin-2-yl)piperidin-4-ol; (R)-1-((4,4-difluorocyclohexyl)methyl)-4-(8-phenyl-7,8-dihydro-6H-pyrrolo[2′,1′:2,3]imidazo[4,5-b]pyridin-2-yl)pyridin-2(1H)-one; (R)-1-(2-methoxyethyl)-4-(8-phenyl-7,8-dihydro-6H-pyrrolo[2′,1′:2,3]imidazo[4,5-b]pyridin-2-yl)pyridin-2(1H)-one; (R)-4-(8-phenyl-7,8-dihydro-6H-pyrrolo[2′,1′:2,3]imidazo[4,5-b]pyridin-2-yl)-1-((tetrahydro-2H-pyran-4-yl)methyl)pyridin-2(1H)-one; 4-((R)-8-phenyl-7,8-dihydro-6H-pyrrolo[2′,1′:2,3]imidazo[4,5-b]pyridin-2-yl)-1-(tetrahydrofuran-3-yl)pyridin-2(1H)-one; (R)-4-(8-phenyl-7,8-dihydro-6H-pyrrolo[2′,1′:2,3]imidazo[4,5-b]pyridin-2-yl)-1-(tetrahydro-2H-pyran-4-yl)pyridin-2(1H)-one; (R)-8-phenyl-2-(1-(tetrahydro-2H-pyran-4-yl)-1H-pyrazol-4-yl)-7,8-dihydro-6H-pyrrolo[2′,1′:2,3]imidazo[4,5-b]pyridine; 4-((4-(6′,8′-dihydro-2H-spiro[benzofuran-3,9′-pyrido[3′,2′:4,5]imidazo[2,1-c][1,4]oxazin]-2′-yl)pyridin-2-yl)oxy)cyclohexanol; (R)-1-(5-(3-fluoro-8-phenyl-7,8-dihydro-6H-pyrrolo[2′,1′:2,3]imidazo[4,5-b]pyridin-2-yl)pyrimidin-2-yl)piperidin-4-ol; 2-(2-morpholinopyrimidin-5-yl)-9-phenyl-7,9-dihydro-6H-pyran[4′,3′:4,5]imidazo[1,2-b]pyridazine; trans-4-((4-(4-(2-methoxyphenyl)-3,4-dihydro-2H-pyran[2′,3′:4,5]imidazo[1,2-a]pyridin-7-yl)pyridin-2-yl)oxy)cyclohexanol; (8aS)-7-(5-(9-phenyl-8,9-dihydro-6H-pyrido[3′,2′:4,5]imidazo[2,1-c][1,4]oxazin-2-yl)pyrimidin-2-yl)hexahydroimidazo[1,5-a]pyrazin-3(2H)-one; 3,3-difluoro-1-(5-(9-phenyl-8,9-dihydro-6H-pyrido[3′,2′:4,5]imidazo[2,1-c][1,4]oxazin-2-yl)pyrimidin-2-yl)piperidin-4-ol; 1-(5-(9-phenyl-8,9-dihydro-6H-pyrido[3′,2′:4,5]imidazo[2,1-c][1,4]oxazin-2-yl)pyrimidin-2-yl)piperidin-4-ol; 2-(2-(1,4-oxazepan-4-yl)pyrimidin-5-yl)-9-phenyl-8,9-dihydro-6H-pyrido[3′,2′:4,5]imidazo[2,1-c][1,4]oxazine; (1-(5-(9-phenyl-8,9-dihydro-6H-pyrido[3′,2′:4,5]imidazo[2,1-c][1,4]oxazin-2-yl)pyrimidin-2-yl)piperidin-3-yl)methanol; (4-fluoro-1-(5-(9-phenyl-8,9-dihydro-6H-pyrido[3′,2′:4,5]imidazo[2,1-c][1,4]oxazin-2-yl)pyrimidin-2-yl)piperidin-4-yl)methanol; 2-hydroxy-1-(4-(5-(9-phenyl-8,9-dihydro-6H-pyrido[3′,2′:4,5]imidazo[2,1-c][1,4]oxazin-2-yl)pyrimidin-2-yl)piperazin-1-yl)propan-1-one; 2-hydroxy-1-(4-(5-(9-phenyl-8,9-dihydro-6H-pyrido[3′,2′:4,5]imidazo[2,1-c][1,4]oxazin-2-yl)pyrimidin-2-yl)piperazin-1-yl)ethanone; (R)-8-phenyl-2-(2-((tetrahydro-2H-pyran-4-yl)methoxy)pyridin-4-yl)-7,8-dihydro-6H-pyrrolo[2′,1′:2,3]imidazo[4,5-b]pyridine; (R)-2-(2-(2-methoxyethoxy)pyridin-4-yl)-8-phenyl-7,8-dihydro-6H-pyrrolo[2′,1′:2,3]imidazo[4,5-b]pyridine; (R)-2-methyl-1-((4-(8-phenyl-7,8-dihydro-6H-pyrrolo[2′,1′:2,3]imidazo[4,5-b]pyridin-2-yl)pyridin-2-yl)oxy)propan-2-ol; (R)-8-phenyl-2-(1,2,3,6-tetrahydropyridin-4-yl)-7,8-dihydro-6H-pyrrolo[2′,1′:2,3]imidazo[4,5-b]pyridine; (1S,4s)-4-(((4-((R)-8-phenyl-7,8-dihydro-6H-pyrrolo[2′,1′:2,3]imidazo[4,5-b]pyridin-2-yl)pyridin-2-yl)oxy)methyl)cyclohexanol; (1R,4r)-4-(((4-((R)-8-phenyl-7,8-dihydro-6H-pyrrolo[1′:22′,1:2,3]imidazo[4,5-b]pyridin-2-yl)pyridin-2-yl)oxy)methyl)cyclohexanol; ((1R,4r) 4-(((4-((R)-8-phenyl-7,8-dihydro-6H-pyrrolo[2′,1′:2,3]imidazo[4,5-b]pyridin-2-yl)pyridin-2-yl)oxy)methyl)cyclohexyl)methanol; (R)-8-Phenyl-2-(1-(pyrimidin-4-yl)-1,2,3,6-tetrahydropyridin-4-yl)-7,8-dihydro-6H-pyrrolo[2′,1′:2,3]imidazo[4,5-b]pyridine; methyl 2-(4-((R)-8-phenyl-7,8-dihydro-6H-pyrrolo[2′,1′:2,3]imidazo[4,5-b]pyridin-2-yl)cyclohex-3-en-1-yl)acetate; 1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; (S)-1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; (R)-1-(5-(8-methyl-8-phenyl-7,8-dihydro-6H-pyrrolo[2′,1′:2,3]imidazo[4,5-b]pyridin-2-yl)pyrimidin-2-yl)piperidin-4-ol; (S)-2-(5-(8-methyl-8-phenyl-7,8-dihydro-6H-pyrrolo[2′,1′:2,3]imidazo[4,5-b]pyridin-2-yl)pyrimidin-2-yl)propan-2-ol; (R)-2-(5-(8-methyl-8-phenyl-7,8-dihydro-6H-pyrrolo[2′,1′:2,3]imidazo[4,5-b]pyridin-2-yl)pyrimidin-2-yl)propan-2-ol; (R)-2-(5-(8-phenyl-7,8-dihydro-6H-pyrrolo[2′,1′:2,3]imidazo[4,5-b]pyridin-2-yl)pyrimidin-2-yl)propan-2-amine; 2-(2-(4,4-difluoropiperidin-1-yl)pyrimidin-5-yl)-9-phenyl-8,9-dihydro-6H-pyrido-[3′,2′:4,5]imidazo[2,1-c][1,4]oxazine; or (1R,4R)-4-((4-((R)-8-phenyl-7,8-dihydro-6H-pyrrolo[2′,1′:2,3]imidazo[4,5-b]pyridin-2-yl)pyridin-2-yl)oxy)cyclohexanol. In certain embodiments, the compound is selected from the group consisting of: 2-(5-(9-phenyl-6,7,8,9-tetrahydro-6,8-methanoimidazo[1,2-a:5,4-b′]dipyridin-2-yl)pyrimidin-2-yl)propan-2-ol; 9-phenyl-2-(2-((tetrahydro-2H-pyran-4-yl)oxy)pyridin-4-yl)-6,7,8,9-tetrahydro-6,8-methanoimidazo[1,2-a:5,4-b′]dipyridine; 4-(5-(9-phenyl-6,7,8,9-tetrahydro-6,8-methanoimidazo[1,2-a:5,4-b′]dipyridin-2-yl)pyrimidin-2-yl)morpholine; 1-(5-(9-phenyl-6,7,8,9-tetrahydro-6,8-methanoimidazo[1,2-a:5,4-b′]dipyridin-2-yl)pyrimidin-2-yl)piperidin-4-ol; 2-(5-((6R,8S,9S)-9-phenyl-6,7,8,9-tetrahydro-6,8-epoxyimidazo[1,2-a:5,4-b′]dipyridin-2-yl)pyrimidin-2-yl)propan-2-ol; 1-(5-((6R,8S,9S)-9-phenyl-6,7,8,9-tetrahydro-6,8-epoxyimidazo[1,2-a:5,4-b′]dipyridin-2-yl)pyrimidin-2-yl)piperidin-4-ol; 2-(5-(9-phenyl-6,7,8,9-tetrahydro-6,8-methanoimidazo[1,2-a:5,4-b′]dipyridin-2-yl)pyrimidin-2-yl)propan-2-ol; 9-phenyl-2-(2-((tetrahydro-2H-pyran-4-yl)oxy)pyridin-4-yl)-6,7,8,9-tetrahydro-6,8-methanoimidazo[1,2-a:5,4-b′]dipyridine; 1-(5-(9-phenyl-6,7,8,9-tetrahydro-6,8-methanoimidazo[1,2-a:5,4-b′]dipyridin-2-yl)pyrimidin-2-yl)piperidin-4-ol; 4-(5-(9-phenyl-6,7,8,9-tetrahydro-6,8-methanoimidazo[1,2-a:5,4-b′]dipyridin-2-yl)pyrimidin-2-yl)morpholine; 2-(5-((6R,8S,9S)-9-phenyl-6,7,8,9-tetrahydro-6,8-epoxyimidazo[1,2-a:5,4-b′]dipyridin-2-yl)pyrimidin-2-yl)propan-2-ol; or 1-(5-((6R,8S,9S)-9-phenyl-6,7,8,9-tetrahydro-6,8-epoxyimidazo[1,2-a:5,4-b′]dipyridin-2-yl)pyrimidin-2-yl)piperidin-4-ol. The disclosures of U.S. Pat. Nos. 10,266,532 B2 and 9,856,253 B2, and U.S. Patent Applications No. US20160304517A1 and US2018/0179198 A1, are incorporated herein in their entireties by reference.

In certain embodiments, the TNF-alpha Targeting Ligand is selected from the group consisting of: 3-(2-(Difluoromethoxy)phenyl)-6-(2-morpholinopyrimidin-5-yl)-2,3-dihydropyrazolo[1,2-a]indazol-9(1H)-one; (R)-3-(2-(difluoromethoxy)phenyl)-6-(2-morpholinopyrimidin-5-yl)-2,3-dihydropyrazolo[1,2-a]indazol-9(1H)-one; (S)-3-(2-(difluoromethoxy)phenyl)-6-(2-morpholinopyrimidin-5-yl)-2,3-dihydropyrazolo[1,2-a]indazol-9(1H)-one; 1-(5-(3-(2-(difluoromethoxy)phenyl)-9-oxo-1,2,3,9-tetrahydropyrazolo[1,2-a]indazol-6-yl)pyrimidin-2-yl)piperidine-4-carboxylic acid; (S)-3-(2-(difluoromethoxy)phenyl) 6-(2-((R)-2-(methoxymethyl)pyrrolidin-1-yl)pyrimidin-5-yl)-2,3-dihydropyrazolo[1,2-a]indazol-9(1H)-one; (R)-3-(2-(difluoromethoxy)phenyl)-6-(2-((R)-2-(methoxymethyl)pyrrolidin-1-yl)pyrimidin-5-yl)-2,3-dihydropyrazolo[1,2-a]indazol-9(1H)-one; (R)-3-(2-(difluoromethoxy)phenyl)-6-(2-(((R)-tetrahydrofuran-3-yl)amino)pyrimidin-5-yl)-2,3-dihydropyrazolo[1,2-a]indazol-9(1H)-one; (S)-3-(2-(difluoromethoxy)phenyl)-6-(2-(((R)-tetrahydrofuran-3-yl)amino)pyrimidin-5-yl)-2,3-dihydropyrazolo[1,2-a]indazol-9(1H)-one; (R) 3-(2-(difluoromethoxy)phenyl)-6-(2-((R)-2-(hydroxymethyl)morpholino)pyrimidin-5-yl)-2,3-dihydropyrazolo[1,2-a]indazol-9(1H)-one; (S)-3-(2-(difluoromethoxy)phenyl)-6-(2-((R)-2-(hydroxymethyl)morpholino)pyrimidin-5-yl)-2,3-dihydropyrazolo[1,2-a]indazol-9(1H)-one; (S) 3-(2-(difluoromethoxy)phenyl)-6-(2-((S)-2-(hydroxymethyl)morpholino)pyrimidin-5-yl)-2,3-dihydropyrazolo[1,2-a]indazol-9(1H)-one; (R)-3-(2-(difluoromethoxy)phenyl)-6-(2-((S)-2-(hydroxymethyl)morpholino)pyrimidin-5-yl)-2,3-dihydropyrazolo[1,2-a]indazol-9(1H)-one; (R) 3-(2-(Difluoromethoxy)phenyl)-6-(2-(2-hydroxypropan-2-yl)pyrimidin-5-yl)-2,3-dihydropyrazolo[1,2-a]indazol-9(1H)-one; (S)-3-(2-(difluoromethoxy)phenyl)-6-(2-(2-hydroxypropan-2-yl)pyrimidin-5-yl)-2,3-dihydropyrazolo[1,2-a]indazol-9(1H)-one; 3-(2-(difluoromethoxy)phenyl)-6-(2-(4-hydroxypiperidin-1-yl)pyrimidin-5-yl)-2,3-dihydropyrazolo[1,2-a]indazol-9(1H)-one; (R)-3-(2-(difluoromethoxy)phenyl)-6-(2-((((S)-5-oxopyrrolidin-3-yl)methyl)amino)pyrimidin-5-yl)-2,3-dihydropyrazolo[1,2-a]indazol-9(1H)-one; (S)-3-(2-(difluoromethoxy)phenyl)-6-(2-((((S)-5-oxopyrrolidin-3-yl)methyl)amino)pyrimidin-5-yl)-2,3-dihydropyrazolo[1,2-a]indazol-9(1H)-one; (R)-3-(2-(difluoromethoxy)phenyl)-6-(2-((((R)-5-oxopyrrolidin-3-yl)methyl)amino)pyrimidin-5-yl)-2,3-dihydropyrazolo[1,2-a]indazol-9(1H)-one; (S)-3-(2-(difluoromethoxy)phenyl)-6-(2-((((R)-5-oxopyrrolidin-3-yl)methyl)amino)pyrimidin-5-yl)-2,3-dihydropyrazolo[1,2-a]indazol-9(1H)-one; (R)-3-(2-(difluoromethoxy)phenyl)-6-(2-(2-hydroxy-7-azaspiro[3.5]nonan-7-yl)pyrimidin-5-yl)-2,3-dihydropyrazolo[1,2-a]indazol-9(1H)-one; (S)-3-(2-(difluoromethoxy)phenyl)-6-(2-(2-hydroxy-7-azaspiro[3.5]nonan-7-yl)pyrimidin-5-yl)-2,3-dihydropyrazolo[1,2-a]indazol-9(1H)-one; (S)-3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one; (S)-3-(2-(difluoromethoxy)phenyl)-6-(2-((R)-3-oxohexahydroimidazo[1,5-a]pyrazin-7(1H)-yl)pyrimidin-5-yl)-2,3-dihydropyrazolo[1,2-a]indazol-9(1H)-one; (R)-3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one; (R)-3-(2-(difluoromethoxy)phenyl)-6-(2-((R)-3-oxohexahydroimidazo[1,5-a]pyrazin-7(1H)-yl)pyrimidin-5-yl)-2,3-dihydropyrazolo[1,2-a]indazol-9(1H)-one; (R)-3-(2-(Difluoromethoxy)phenyl)-6-(2-((3-methoxypropyl)amino)pyrimidin-5-yl)-2,3-dihydropyrazolo[1,2-a]indazol-9(1H)-one; (S)-3-(2-(Difluoromethoxy)phenyl)-6-(2-((3-methoxypropyl)amino)pyrimidin-5-yl)-2,3-dihydropyrazolo[1,2-a]indazol-9(1H)-one; (S)-6-(2-(4-Acetylpiperazin-1-yl)pyrimidin-5-yl)-3-(2-(difluoromethoxy)phenyl)-2,3-dihydropyrazolo[1,2-a]indazol-9(1H)-one; (R)-6-(2-(4-Acetylpiperazin-1-yl)pyrimidin-5-yl)-3-(2-(difluoromethoxy)phenyl)-2,3-dihydropyrazolo[1,2-a]indazol-9(1H)-one; 6-(2-(difluoromethoxy)phenyl)-3-(2-morpholinopyrimidin-5-yl)-8,9-dihydro-6H-pyridazino[1,2-a]indazol-11(7H)-one; 6-(2-(difluoromethoxy)phenyl)-3-(2-(4-hydroxypiperidin-1-yl)pyrimidin-5-yl)-8,9-dihydro-6H-pyridazino[1,2-a]indazol-11(7H)-one; 6-(2-(difluoromethoxy)phenyl)-3-(2-(1,1-dioxidothiomorpholino)pyrimidin-5-yl)-8,9-dihydro-6H-pyridazino[1,2-a]indazol-11(7H)-one; 3-(2-methoxyphenyl)-6-(2-morpholinopyrimidin-5-yl)-2,3-dihydropyrazolo[1,2-a]indazol-9(1H)-one; (R)-6-(2-(2-hydroxypropan-2-yl)pyrimidin-5-yl)-3-(2-methoxyphenyl)-2,3-dihydropyrazolo[1,2-a]indazol-9(1H)-one; (S)-6-(2-(2-hydroxypropan-2-yl)pyrimidin-5-yl)-3-(2-methoxyphenyl)-2,3-dihydropyrazolo[1,2-a]indazol-9(1H)-one; 2-methyl-6-(6-(2-morpholinopyrimidin-5-yl)-9-oxo-1,2,3,9-tetrahydropyrazolo[1,2-a]indazol-3-yl)benzonitrile; 6-(2-morpholinopyrimidin-5-yl)-3-phenyl-2,3-dihydropyrazolo[1,2-a]indazol-9(1H)-one; 4-methoxy-3-(6-(2-morpholinopyrimidin-5-yl)-9-oxo-1,2,3,9-tetrahydropyrazolo[1,2-a]indazol-3-yl)benzonitrile; 2-methoxy-3-(6-(2-morpholinopyrimidin-5-yl)-9-oxo-1,2,3,9-tetrahydropyrazolo[1,2-a]indazol-3-yl)benzonitrile; 3-(1-isopropyl-1H-pyrazol-5-yl)-6-(2-morpholinopyrimidin-5-yl)-2,3-dihydropyrazolo[1,2-a]indazol-9(1H)-one; 2-methyl-6-(6-(2-morpholinopyrimidin-5-yl)-9-oxo-1,2,3,9-tetrahydropyrazolo[1,2-a]indazol-3-yl)benzamide; rac-(1R,9bR)-1-(2-(difluoromethoxy)phenyl)-8-(2-morpholinopyrimidin-5-yl)-2,3-dihydro-1H-pyrrolo[2,1-a]isoindol-5(9bH)-one; rac-(1R,9bS)-1-(2-(difluoromethoxy)phenyl)-8-(2-morpholinopyrimidin-5-yl)-2,3-dihydro-1H-pyrrolo[2,1-a]isoindol-5(9bH)-one; rac-(1R,10bR) 1-(2-(difluoromethoxy)phenyl)-9-(2-morpholinopyrimidin-5-yl)-3,4-dihydro-1H-[1,4]oxazino[3,4-a]isoindol-6(10bH)-one; (1S,9bS)-1-(2-(difluoromethoxy)phenyl)-8-(2-morpholinopyrimidin-5-yl)-2,3-dihydro-1H-pyrrolo[2,1-a]isoindol-5(9bH)-one; (1R,9bR)-1-(2-(difluoromethoxy)phenyl)-8-(2-morpholinopyrimidin-5-yl)-2,3-dihydro-1H-pyrrolo[2,1-a]isoindol-5(9bH)-one; (1S,9bS)-1-(2-(difluoromethoxy)phenyl)-8-(2-(2-hydroxypropan-2-yl)pyrimidin-5-yl)-2,3-dihydro-1H-pyrrolo[2,1-a]isoindol-5(9bH)-one; (1R,9bR)-1-(2-(difluoromethoxy)phenyl)-8-(2-(2-hydroxypropan-2-yl)pyrimidin-5-yl)-2,3-dihydro-1H-pyrrolo[2,1-a]isoindol-5(9bH)-one; (1S,9bS)-1-(2-(difluoromethoxy)phenyl)-8-(2-((R)-2-(methoxymethyl)pyrrolidin-1-yl)pyrimidin-5-yl)-2,3-dihydro-1H-pyrrolo[2,1-a]isoindol-5(9bH)-one; (1R,9bR)-1-(2-(difluoromethoxy)phenyl)-8-(2-((R)-2-(methoxymethyl)pyrrolidin-1-yl)pyrimidin-5-yl)-2,3-dihydro-1H-pyrrolo[2,1-a]isoindol-5(9bH)-one; (1R,9bR)-1-(2-(difluoromethoxy)phenyl)-8-(2-((R)-3-oxohexahydroimidazo[1,5-a]pyrazin-7(1H)-yl)pyrimidin-5-yl)-2,3-dihydro-1H-pyrrolo[2,1-a]isoindol-5(9bH)-one; (1R,9bR)-1-(2-(difluoromethoxy)phenyl)-8-(2-((S)-3-oxohexahydroimidazo[1,5-a]pyrazin-7(1H)-yl)pyrimidin-5-yl)-2,3-dihydro-1H-pyrrolo[2,1-a]isoindol-5(9bH)-one; (1R)-1-(2-(difluoromethoxy)phenyl)-8-(2-(2-hydroxypropan-2-yl)-4-methylpyrimidin-5-yl)-2,3-dihydro-1H-pyrrolo[2,1-a]isoindol-5(9bH)-one; (R)-6-(2-((R)-4-acetyl-2-methylpiperazin-1-yl)pyrimidin-5-yl)-3-(2-(difluoromethoxy)phenyl)-2,3-dihydropyrazolo[1,2-a]indazol-9(1H)-one; (S)-3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one; (R)-3-(2-(difluoromethoxy)-5-methylphenyl)-6-(2-(2-hydroxypropan-2-yl)pyrimidin-5-yl)-2,3-dihydropyrazolo[1,2-a]indazol-9(1H)-one; (R)-6-(2-((R)-4-acetyl-3-methylpiperazin-1-yl)pyrimidin-5-yl)-3-(2-(difluoromethoxy)phenyl)-2,3-dihydropyrazolo[1,2-a]indazol-9(1H)-one; (R)-3-(2-(difluoromethoxy)-5-methylphenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one; (R)-3-(2-(difluoromethoxy)phenyl)-6-(2-((R)-4-(2-hydroxyacetyl)-3-methylpiperazin-1-yl)pyrimidin-5-yl)-2,3-dihydropyrazolo[1,2-a]indazol-9(1H)-one; (R)-3-(2-(difluoromethoxy)-5-methylphenyl) 6-(2-(2-hydroxypropan-2-yl)pyrimidin-5-yl)-2,3-dihydropyrazolo[1,2-a]indazol-9(1H)-one; (S) 3-(2-(difluoromethoxy)phenyl)-6-(2-((S)-3-oxohexahydroimidazo[1,5-a]pyrazin-7(1H)-yl)pyrimidin-5-yl)-2,3-dihydropyrazolo[1,2-a]indazol-9(1H)-one; (S)-3-(2-(difluoromethoxy)phenyl)-6-(2-(2-hydroxypropan-2-yl)pyrimidin-5-yl)-2,3-dihydropyrazolo[1,2-a]indazol-9(1H)-one; (R)-3-(2-(difluoromethoxy)phenyl)-6-(2-((R)-3-hydroxy-4-(2-hydroxyacetyl)piperazin-1-yl)pyrimidin-5-yl)-2,3-dihydro-1H,9H-pyrazolo[1,2-a]indazol-9-one; (S)-3-(2-(difluoromethoxy)-5-methylphenyl)-7-fluoro-6-(2-(2-hydroxypropan-2-yl)pyrimidin-5-yl)-2,3-dihydropyrazolo[1,2-a]indazol-9(1H)-one; (R)-3-(2-(difluoromethoxy) 5-methylphenyl)-7-fluoro-6-(2-(2-hydroxypropan-2-yl)pyrimidin-5-yl)-2,3-dihydropyrazolo[1,2-a]indazol-9(1H)-one; (R)-3-(2-(difluoromethoxy)phenyl)-7-fluoro-6-(2-(2-hydroxypropan-2-yl)pyrimidin-5-yl)-2,3-dihydropyrazolo[1,2-a]indazol-9(1H)-one; (S)-3-(2-(difluoromethoxy)phenyl)-7-fluoro-6-(2-(2-hydroxypropan-2-yl)pyrimidin-5-yl)-2,3-dihydropyrazolo[1,2-a]indazol-9(1H)-one; 3-(5-(hydroxymethyl)-2-methoxyphenyl)-6-(2-morpholinopyrimidin-5-yl)-2,3-dihydropyrazolo[1,2-a]indazol-9(1H)-one; (R)-3-(2-(difluoromethoxy)-5-methylphenyl)-7-fluoro-6-(2-(2-hydroxypropan-2-yl)pyrimidin-5-yl)-2,3-dihydro-1H,9H-pyrazolo[1,2-a]indazol-9-one; (S)-3-(2-(difluoromethoxy)-5-methylphenyl)-7-fluoro-6-(2-(2-hydroxypropan-2-yl)pyrimidin-5-yl)-2,3-dihydro-1H,9H-pyrazolo[1,2-a]indazol-9-one; (S)-3-(2-(difluoromethoxy)phenyl)-6-(2-(2-hydroxypropan-2-yl)pyrimidin-5-yl)-2,3-dihydro-1H,9H-pyrazolo[1,2-a]indazol-9-one; (R)-3-(2-(difluoromethoxy)-5-methylphenyl)-6-(2-((S)-3-oxohexahydroimidazo[1,5-a]pyrazin-7(1H)-yl)pyrimidin-5-yl)-2,3-dihydro-1H,9H-pyrazolo[1,2-a]indazol-9-one; (R)-3-(2-(difluoromethoxy)-5-methylphenyl)-6-(2-(2-hydroxypropan-2-yl)pyrimidin-5-yl)-2,3-dihydro-1H,9H-pyrazolo[1,2-a]indazol-9-one; (R)-6-(2-((R)-4-acetyl-3-methylpiperazin-1-yl)pyrimidin-5-yl)-3-(2-(difluoromethoxy)phenyl)-2,3-dihydropyrazolo[1,2-a]indazol-9(1H)-one; (R)-3-(2-(difluoromethoxy)phenyl)-6-(2-((R)-3-hydroxy-4-(2-hydroxyacetyl)piperazin-1-yl)pyrimidin-5-yl)-2,3-dihydropyrazolo[1,2-a]indazol-9(1H)-one; 9b-(2-methoxyphenyl)-8-(2-morpholinopyrimidin-5-yl)-2,3-dihydro-1H-pyrrolo[2,1-a]isoindol-5(9bH)-one; or 3-(5-(hydroxymethyl)-2-methoxyphenyl)-6-(2-morpholinopyrimidin-5-yl)-2,3-dihydropyrazolo[1,2-a]indazol-9(1H)-one.

### Prostate Specific Membrane Antigen (PSMA)

In some embodiments, the Target Extracellular Protein is human prostate specific membrane antigen (UniProtKB—Q04609 (FOLH1_HUMAN)), also known as Glutamate carboxypeptidase II (GCPII), N-acetyl-L-aspartyl-L-glutamate peptidase I (NAALADase I), and NAAG peptidase. PSMA is an enzyme that catalyzes the reaction of N-Acetyl aspartylglutamate to glutamate and N-acetylaspartate. PSMA inhibitors have been shown to decrease the levels of glutamate in the nervous system, protecting from neural degeneration in models of stroke, amyotrophic lateral sclerosis (ALS) and neuropathic pain. In certain embodiments, the Extracellular Protein Targeting Ligand binds to PSMA.

Nonlimiting examples of PSMA ligands include

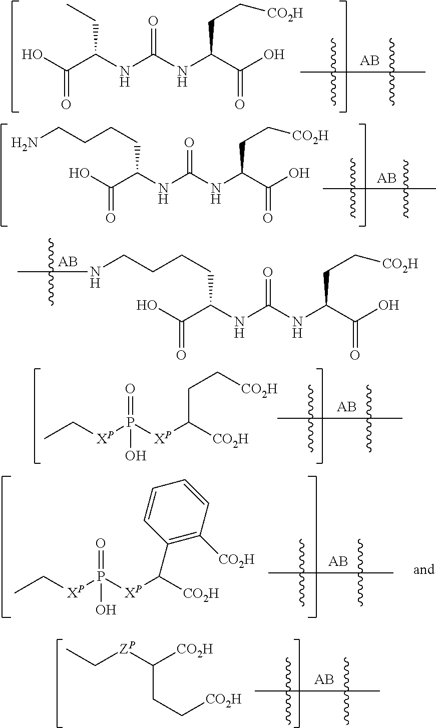

wherein
X^{P} and Y^{P} are each independently CH₂, O, NH, or S; and
Z^{P} is O, CH₂, NR⁶, S(O), S(O)₂, S(O)₂O, OS(O)₂, or OS(O)₂O.

### Envelope Glycoprotein GP120

In certain embodiments, the gp120 binding ligand is compound disclosed in WO 2001/062255 or WO 2003/072028. In certain embodiments, the gp120 binding ligand is a

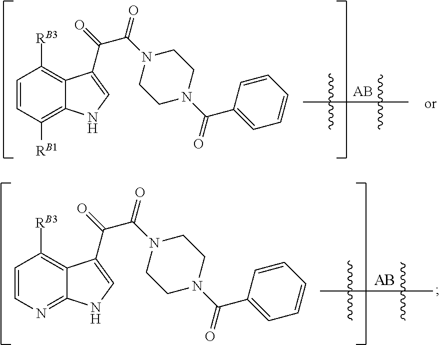

wherein
R^{B1} is selected from hydrogen, C₁-C alkyl, —O—C₁-C₆alkyl, aryl, and heteroaryl;
in certain embodiments, R^{B1} is phenyl, naphthyl, pyridyl (2-, 3- or 4-pyridyl group), thiazolyl (2-, 4- or 5-thiazole), isothiazolyl, oxazolyl (2-, 4- or 5-oxazole), isoxazolyl, furanyl (2- or 3-furan) or thiophenyl (2- or 3-thiophene);
R^{B2} is hydrogen or C₁-C₃alkyl; and
R^{B3} is selected from hydrogen, C₁-C₆alkyl, —O—, —NR⁶—, —S(O)—, —S(O)₂—, —S(O)₂O—, and —OS(O)₂O—.

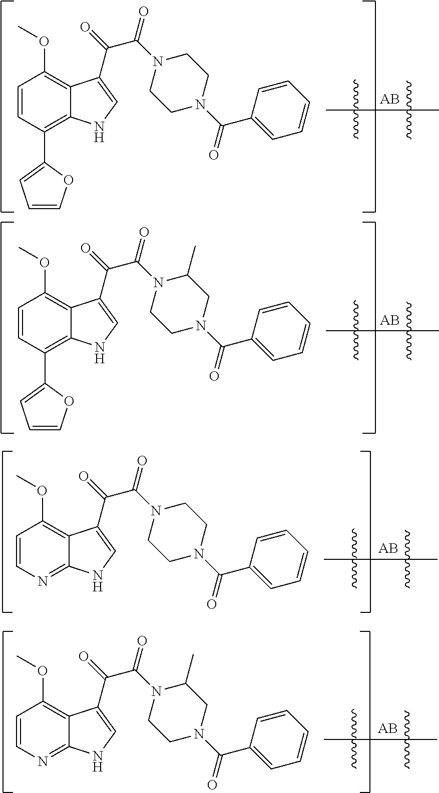

In certain embodiments, the Target Protein is selected from Serum amyloid P component, amyloid precursor protein, C reactive protein (CRP), an N-methyl-D-aspartate (NMD A) receptor, a-synuclein, IAPP, transthyretin, and combinations thereof. In some embodiments, the Target Protein is selected from a calcitonin gene-related peptide (CGRP), a CGRP receptor, an N-methyl-D-aspartate (NMD A) receptor, myeloperoxidase (MPO), IAPP, transthyretin, extracellular tau, beta-amyloid, amyloid precursor protein, prion protein, and a-synuclein. In some embodiments, the Extracellular Protein Targeting Ligand binds to extracellular tau, beta-amyloid, amyloid precursor protein, prion protein, a-synuclein, or a combination thereof.

In certain embodiments, the Extracellular Protein Targeting Ligand comprises one of the following amino acid sequences that binds extracellular tau:
VY-WIW: SVWIWYE (SEQ ID NO:418), (Seidler, P. M. et al, Journal of Biological Chemistry, 2019, 29:16451-16464); or
IN-M4: DVWIINKKLK (SEQ ID NO:419), (Seidler, P. M. et al, Journal of Biological Chemistry, 2019, 29:16451-16464), wherein SEQ ID NOs 418 and 419 can be attached to the Linker through the C or N terminus.

In certain embodiments, the Extracellular Protein Targeting Ligand comprises one of the following amino acid sequences that targets amyloid beta:
NCAM1 (N): MLRTKDLIWTLFFLGTAVS-NH₂ (SEQ ID NO:420), (Henning-Knechtel, A. et al, Cell Reports Physical Science, 2020, 26:100014);
N-Pr: MLRTKDLIWTLFFLGTAVSKKRPKP-NH₂ (SEQ ID NO:421), (Henning-Knechtel, A. et al, Cell Reports Physical Science, 2020, 26:100014); or
N-Ab: MLRTKDLIWTLFFLGTAVSKKLVFF-NFb (SEQ ID NO:422), (Henning-Knechtel, A. et al, Cell Reports Physical Science, 2020, 26:100014), wherein SEQ ID NOs 420-422 can be attached to the Linker through the C or N terminus.

In certain embodiments, the amino end of any of SEQ ID NOs: 418-422 binds to the Linker group or the LPR1 binding motif In other embodiments, the carboxylic acid end of any of SEQ ID NOs: 418-422 binds to the Linker. In certain embodiments, the carboxylic acid terminus of any of SEQ ID NOs: 418-422 is a carboxamide group and the amine terminus is covalently linked to the Linker.

In certain embodiments, the Target Protein Binding Ligand binds to the N-methyl-D-aspartate (NMD A) receptor. Non limiting examples of a Target Protein Binding Ligand that bind to the N-methyl-D-aspartate (NMD A) receptor include

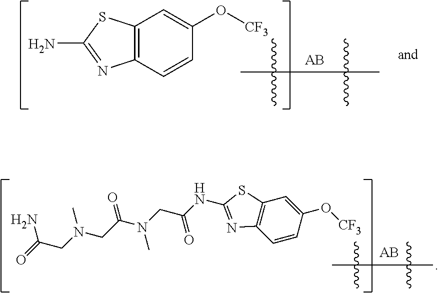

In certain embodiments, the Target Protein Binding Ligand binds prions. Non limiting examples of a Target Protein Binding Ligand that binds a prion include

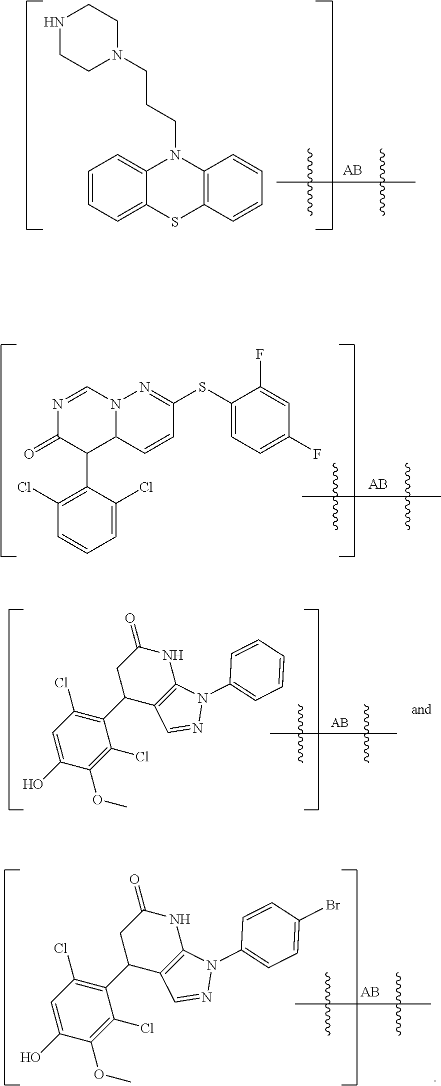

In certain embodiments, the Target Protein Binding Ligand binds prions. Non limiting examples of a Target Protein Binding Ligand that binds a prion include

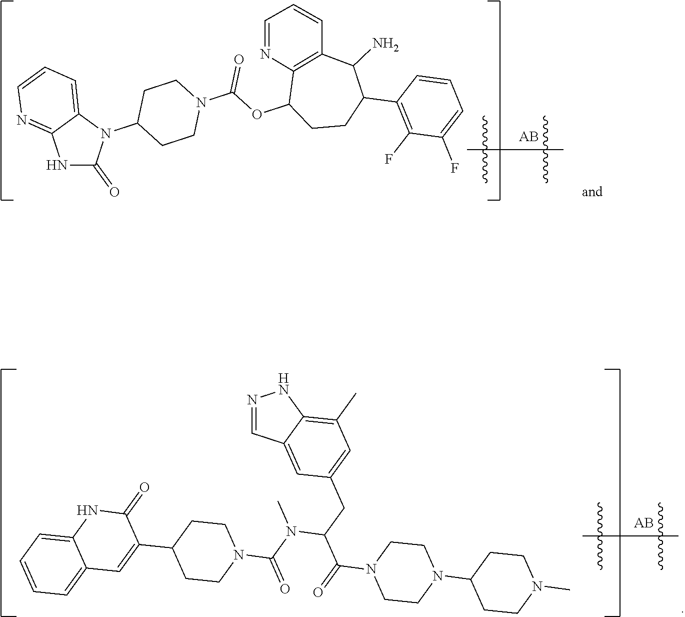

### Anti-β1AR Antibodies

In certain embodiments, the Target Protein Binding Ligand binds autoantibodies to the β1 adrenergic receptor (anti-β1AR antibodies). Non limiting examples of ligands that bind anti-β1AR antibodies include the peptide DEARRCYNDPKCSDFVQ (SEQ ID NO:423), a peptide that is at least 94% homologous to SEQ ID NO:423, a peptide that is at least 88% homologous to SEQ ID NO:423, a peptide that is at least 82% homologous to SEQ ID NO:423, and a peptide that is at least 76% homologous to SEQ ID NO:423.

### CD16a (FcγRIII)

In certain embodiments, the Target Protein Binding Ligand binds CD16a. In some embodiments, CD16a is a high-affinity form, e.g., 158V. In some embodiments, CD16a is a low-affinity form, e.g., 158F. Nonlimiting examples of ligands that bind CD16a include

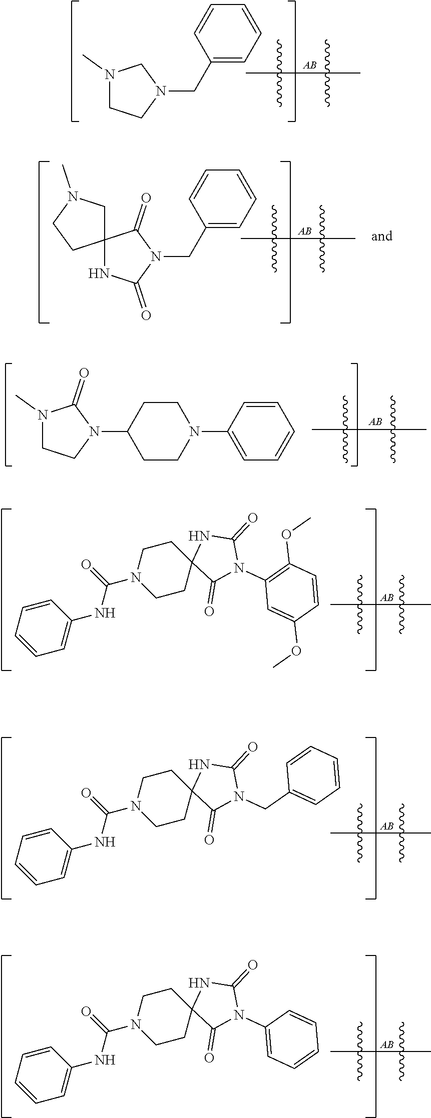

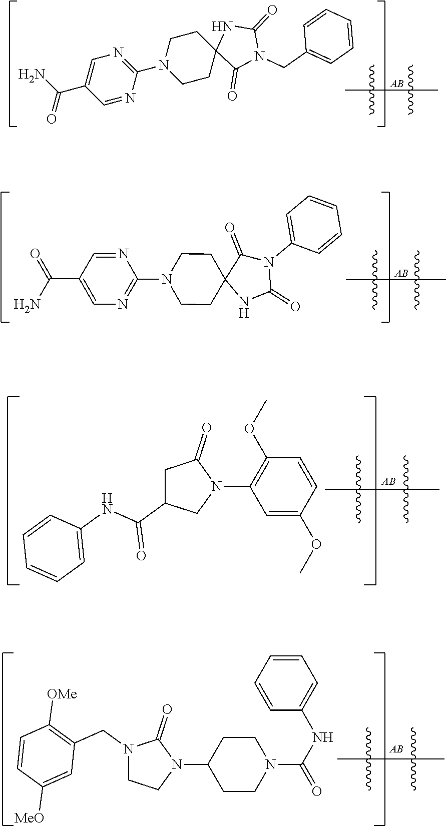

### Toll-Like Receptors

Toll-like receptors (TLRs) are receptors that recognize pathogens via pathogen-associated molecular patterns. These receptors are a major component of immune response, and activation of TLRs results in proinflammatory signaling. Decreasing TLR activity may be beneficial in the treatment of diseases such as rheumatoid arthritis and systemic lupus erythematosus.

In certain embodiments, the Extracellular Protein Targeting Ligand binds TLRs. In certain embodiments the TLR ligand is Rintatolimod, SMP-105, IPH-3102, CBLB502, MGN-1706, IMO-2055, ANA773, OM-174, ISS1018, Agatolimod, 852A, Imiquimod or Cadi-05. In certain embodiments the TLR ligand is Imiquimod. In certain embodiments, the TLR ligand is a compound described in Bioorg. Med. Chem. Lett. (2010) 6384; Wu, et al. Proc Natl Acad Sci USA, 2007; 104(10):3990-5; Gorden, et al., The Journal of Immunology, 2005; 174(3):1259-68; and Reece, et al., or Proc Natl Acad Sci USA, 2005; 102(42):15190-4.

In certain embodiments, the TLR ligand is selected from

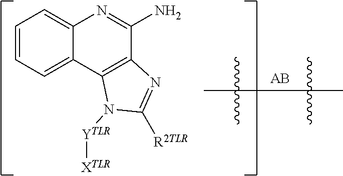

### Wherein

R^{2TLR} is C₁-C₄alkyl, —C₁-C₄—NR⁶—C₁-C₄—, —C₁-C₄—O—C₁-C₄—, and —C₁-C₄—S—C₁-C₄—;
Y^{TLR} is selected from

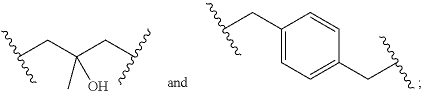

and
X^{TLR} is selected from OH, SH, NH₂, or NH(C₁-C₃alkyl).

In certain embodiments, the TLR ligand is selected from

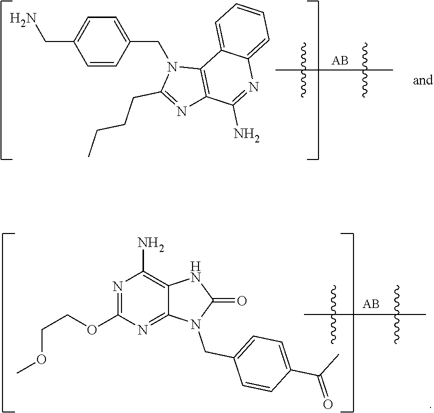

### Soluble FLT-1 (VEGFR-1)

Soluble fms-like tyrosine kinase 1 (sFLT-1), an antiangiogenic protein implicated in the pathogenesis of preeclampsia. This receptor binds VEGFA, VEGFB, and placental growth factor and controls angiogenesis in healthy and diseased tissues. sFLT-1 is upregulated in women with preeclampsia and high levels of sFLT-1 is a cause of maternal hypertension of proteinuria. During placentation, a critical process is remodeling of certain arteries to support the pregnancy. One such artery is the spiral artery (SpA). SpA remodeling involves apoptosis of certain mother cells followed by the production of specialized fetal trophoblast cells in combination with uterine natural killer (uNK) cells surrounding the SpAs. In preeclampsia pregnancies, SpA remodeling is poor and the reduced vasodilation during preeclampsia affects placental perfusion.

In certain embodiments, the Extracellular Protein Targeting Ligand binds sFLT-1. In certain embodiments, the sFLT-1 binding ligand is a fragment or homolog of VEGFA, VEGFB, or placental growth factor (Barleon, B. et al “Mapping the Sites for Ligand Binding and Receptor Dimerization at the Extracellular Domain of the Vascular Endothelial Growth Factor Receptor FLT-1” Protein Chemistry and Structure 1997, 272(16), 10382-10388).

Soluble Endoglin (sEng)

sEng (soluble Eng), caused by Eng shedding from endothelial cell surface, has antiangiogenic effects in pregnant women, which can lead to preeclampsia. It has been hypothesized that this occurs via binding to circulating TGF-β1, a protein involved in homeostasis of angiogenic processes. sEng is used as a biomarker of preeclampsia during pregnancy, in particular during the second trimester. In certain embodiments, the Extracellular Protein Targeting Ligand binds sEng.

In certain embodiments, the Extracellular Protein Targeting Ligand binds a protein target selected from the group consisting of 1-40-β-amyloid, 5′-nucleotidase, activated F9, F10, activin receptor-like kinase 1, alpha-fetoprotein, amyloid, angiopoietin 2, angiopoietin 3, anthrax toxin, AOC3, AOC3 (VAP-1), Bacillus anthracis anthrax, BAFF, beta amyloid, c-Met, C1s, C242 antigen, C5, CA-125, calcitonin, calcitonin gene-related peptide, calcitonin gene-related peptide alpha, Canis lupus familiaris IL31, carbonic anhydrase 9 (CA-IX), CEA, CEA-related antigen, CEACAM5, CFD, CGRP, clumping factor A, coagulation factor III, complement C5a, CSF1, MCSF, CSF2, dabigatran, E. coli shiga toxin type-1, E. coli shiga toxin type-2, EGFL7, EGFR, endotoxin, episialin, FGF 23, fibrin II, beta chain, fibronectin extra domain-B, folate hydrolase, GDF-8, gelatinase B, GMCSF, growth differentiation factor 8, hemagglutinin, hemagglutinin HA, HGF, HIV-1, HNGF, Hsp90, human beta-amyloid, human scatter factor receptor kinase, human TNF, IFN-J, IFN-U, IgE, IgE Fc region, IGF1, IGF2, IGHE, IL 17A, IL 17A and IL 17F, IL 20, IL-1, IL-12, IL-23, IL-13, IL-17, IL-la, IL-22, IL-4, IL-5, IL-6, IL17A and IL17F, IL1A, IL2, IL23, IL23A, IL31RA, IL6, IL6R, IL9, ILGF2, Influenza A hemagglutinin, influenza A virus hemagglutinin, influenza A virus hemagglutinin HA, interferon gamma, interferon gamma-induced protein, interleukin 1 alpha, interleukin 13, interleukin 17 alpha, interleukin 17 alpha, TNF, interleukin 17A, kallikrein, LOXL2, LRRC15, LTA, MASP-2, MCP-1, MIF, MST1R (aka RON), MUC1, myostatin, NACP, NCA-90 (granulocyte antigen), neural apoptosis-regulated proteinase 1, NGF, NOGO-A, Notch 1, NRP1, oxLDL, PCSK9, PD-L1, phosphatidylserine, RANKL, RGMA, root plate-specific spondin 3, RTN4, sclerostin, SDC1, serum amyloid A protein, serum amyloid P component, SOST, Staphylococcus aureus alpha toxin, tau protein, TFPI, TGF beta 1, TGF beta 2, TGF-ü, TNF-I̧, TROP-2, TSLP, VEGF-A, VEGF-A and Ang-2, VEGFA, and VWF.

In certain embodiments, the Extracellular Protein Targeting Ligand is an antibody ligand selected from Abagovomab, Abrezekimab, Adalimumab, Aducanumab, Afasevikumab, Afelimomab, Alirocumab, Altumomab, Altumomab pentetate, Andecaliximab, Anrukinzumab, Arcitumomab, Ascrinvacumab, Atezolizumab, Atidortoxumab, Atinumab, Avelumab, Bapineuzumab, Bavituximab, Belimumab, Bermekimab, Besilesomab, Bevacizumab, Biciromab, Bimekizumab, Birtamimab, Blosozumab, Bococizumab, Brazikumab, Briakinumab, Brodalumab, Brolucizumab, Brontictuzumab, Burosumab, Cabiralizumab, Canakinumab, Cantuzumab, Cantuzumab ravtansine, Caplacizumab, Carlumab, Cergutuzumab, Cergutuzumab amunaleukin, Certolizumab, Certolizumab pegol, Cibisatamab, Clazakizumab, Clivatuzumab, Clivatuzumab tetraxetan, Concizumab, Crenezumab, Dectrekumab, Denosumab, Dezamizumab, Diridavumab, Domagrozumab, Dorlimomab, Dorlimomab aritox, Durvalumab, Dusigitumab, Eculizumab, Edobacomab, Efungumab, Eldelumab, Elezanumab, Elsilimomab, Emactuzumab, Emapalumab, Emicizumab, Enokizumab, Epitumomab, Epitumomab cituxetan, Eptinezumab, Erenumab, Evinacumab, Evolocumab, Faricimab, Fasinumab, Fezakinumab, Ficlatuzumab, Firivumab, Fletikumab, Fontolizumab, Fremanezumab, Fresolimumab, Frovocimab, Frunevetmab, Fulranumab, Galcanezumab, Gantenerumab, Gatipotuzumab, Gedivumab, Gevokizumab, Gimsilumab, Girentuximab, Golimumab, Gosuranemab, Guselkumab, Idarucizumab, Igovomab, Imalumab, Indatuximab, Indatuximab ravtansine, Infliximab, Istiratumab, Ixekizumab, Labetuzumab, Lacnotuzumab, Lampalizumab, Lanadelumab, Landogrozumab, Lebrikizumab, Lemalesomab, Lendalizumab, Lenzilumab, Lerdelimumab, Lesofavumab, Ligelizumab, Lodelcizumab, Lokivetmab, Lutikizumab, Marstacimab, Mepolizumab, Metelimumab, Mirikizumab, Nacolomab, Nacolomab tafenatox, Namilumab, Narnatumab, Navivumab, Naxitamab, Nebacumab, Nemolizumab, NEOD, Nerelimomab, Nesvacumab, Netakimab, Nofetumomab, Nofetumomab merpentan, Obiltoxaximab, Oleclumab, Olendalizumab, Olokizumab, Omalizumab, OMS, Onartuzumab, Oregovomab, Orticumab, Otilimab, Ozanezumab, Ozoralizumab, Parsatuzumab, Pascolizumab, Pasotuxizumab, Pateclizumab, Pemtumomab, Perakizumab, Pexelizumab, Placulumab, Ponezumab, Prasinezumab, Pritoxaximab, Quilizumab, Radretumab, Ralpancizumab, Ranevetmab, Ranibizumab, Ravulizumab, Raxibacumab, REGN-EB, Remtolumab, Reslizumab, Rilotumumab, Risankizumab, Romilkimab, Romosozumab, Rontalizumab, Rosmantuzumab, Sacituzumab, Sacituzumab govitecan, Samrotamab, Samrotamab vedotin, Sarilumab, Secukinumab, Setoxaximab, Setrusumab, Sifalimumab, Siltuximab, Simtuzumab, Sirukumab, Sofituzumab, Sofituzumab, Sofituzumab vedotin, Solanezumab, Sontuzumab, Stamulumab, Sulesomab, Sutimlimab, Suvizumab, Suvratoxumab, Tabalumab, Tacatuzumab, Tacatuzumab tetraxetan, Talizumab, Tanezumab, Tefibazumab, Telimomab, Telimomab aritox, Tesidolumab, Tezepelumab, Tibulizumab, Tildrakizumab, Timolumab, Tisotumab, Tisotumab vedotin, Tralokinumab, Trevogrumab, Urtoxazumab, Ustekinumab, Vanucizumab, Vapaliximab, Varisacumab, Vepalimomab, Vesencumab, Vobarilizumab, Vunakizumab, and Xentuzumab.

In certain embodiments, the Extracellular Protein Targeting Ligand binds a protein target selected from the group consisting of TNFa, HER2, EGFR, HER3, VEGFR, CD20, CD 19, CD22, anb3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPa, CD38, CD73, TGF-b, Bombesin R, CAIX, CD13, CD44v6, Emmprin, Endoglin, EpCAM, FAP-a, Folate R, GRP78, IGF-1R, Matriptase, Mesothelin, sMET/HGFR, MT1-MMP, MT6-MMP, PSCA, PSMA, Tn antigen, and uPAR, TSHRa, Myelin oligodendrocyte glycoprotein (MOG), AChR-a1, noncollagen domain 1 of the a3 chain of type IV collagen (a3NCl), ADAMTS13, Desmoglein-1/3, or GPIb/IX, GPIIb/IIIa, GPIa/IIa, NMDA receptor, glutamic acid decarboxylase (GAD), amphiphysin and gangliosides GM1, GD3, GQ1B, LILRB1, LILRB2, VEGF-R, CXCR4, CXCL12, CSF-1, CD47, aggregated light chain or aggregated transthyretin.

In certain embodiments, the Extracellular Protein Targeting Ligand binds an antibody that binds to TSHRa, MOG, AChR-al, noncollagen domain 1 of the a3 chain of type IV collagen (a3NCl), ADAMTS13, Desmoglein-1/3, or GPIb/IX, GPIIb/IIIa, GPIa/IIa, NMDA receptor, glutamic acid decarboxylase (GAD), amphiphysin, or gangliosides GM1, GD3 or GQ1B.

In certain embodiments, the Extracellular Protein Targeting Ligand binds SIRPa, CCR2, CSF-1R, LILRB1, LILRB2, VEGF-R, or CXCR4. In other embodiments, the target protein associated with TAMs comprises CCL2, CXCL12, CSF-1 or CD47.

In certain embodiments, the Extracellular Protein Targeting Ligand binds a protein that is upregulated in cancer or involved in cancer progression. In some embodiments, the target protein upregulated in cancer or involved in cancer progression comprises HER2, EGFR, HER3, VEGFR CD20, CD19, CD22, anb3 integrin, CEA, CXCR4, MUC1, LCAM1, EphA2, PD-1, PD-L1, TIGIT, TIM3, CTLA4, VISTA, Notch receptors, EGF, c-MET, CCL2, CCR2, Frizzled receptors, Wnt, LRP5/6, CSF-1R, SIRPa, CD38, CD73, or TGF-b.

In certain embodiments, the Extracellular Protein Targeting Ligand binds an autoantibody of an autoimmune disease. In some embodiments, the target protein is an autoantigen in an autoimmune disease. In some embodiments, the autoimmune disease is selected from Graves' Disease, Myelin oligodendrocyte glycoprotein antibody-associated disease (MOGAD), Myasthenia Gravis, Anti-GBM Disease, Immune Thrombotic Thrombocytopenic Purpura, Acquired Pemphigus Vulgaris, Immune Thrombocytopenia, autoimmune encephalitis, Guillain-Barre Syndrome, and Membranous Nephropathy.

In certain embodiments, the Extracellular Protein Targeting Ligand is a ligand that binds an autoantibody which itself binds disintegrin and metalloproteinase with a thrombospondin type 1 motif, member 13 (ADAMTS13), steroidogenic cytochrome P450 enzyme 21-hydroxylase, N-methyl-d-aspartate-(NMDA)-receptor, erythrocytes, anti-smooth muscle antibodies (ASMAs), actin, platelet, signal recognition particle (SRP), 3-hydroxy-3-methyl-glutaryl-coenzyme A reductase (HMGCR), myosin, sperm, amylase alpha2, type XVII collagen (col 17), kallikrein 13, type VII collagen (col7), myeloperoxidase (MPO), type IV collagen, proteinase 3 (PR3), thyrotropin receptor (TSHR), thyroglobulin, thyroid peroxidase (TPO), thyroglobulin, thyroid peroxidase (TPO), platelets, myeloperoxidase (MPO), muscle nicotinic acetylcholine receptors, muscle-specific kinase (MuSK), low-density lipoprotein receptor protein 4 (LRP4), myosin, beta1 adrenergic receptor, adenine-nucleotide translocase, aquaporin-4, myelin oligodendrocyte glycoprotein (MOG), heat shock protein 90 (HSP90), heat shock protein A5 (HSPA5), desmoglein-3, parietal cells, mitochondria, phospholipase A2 receptor (PLA2R), thrombospondin type 1 domain-containing 7A (THSD7A), cyclic citrullinated proteins, RNA binding proteins (Ros), La, double-stranded DNA (dsDNA), angiotensin II type 1 receptor (AT1R), endothelin-1 type A receptor (ETAR), insulin, glutamic acid decarboxylase or protein tyrosine phosphatase.

In some embodiments, the autoantibody in the autoimmune disease is an antibody binding to TSHRa, Myelin oligodendrocyte protein (MOG), AChR-a1, noncollagen domain 1 of the a3 chain of type IV collagen (a3NCl), ADAMTS13, Desmoglein-1/3, GPIb/IX, GPIIb/IIIa, GPIa/IIa, NMDA receptor, glutamic acid decarboxylase (GAD), amphiphysin, or gangliosides GM1, GD3 or GQIB.

In some embodiments, the Extracellular Protein Targeting Ligand binds a protein that is upregulated or expressed in a neurodegenerative disease. In some embodiments, the target protein upregulated or expressed in a neurodegenerative disease is alpha-synuclein, amyloid beta or complement cascade component.

In some embodiments, the Extracellular Protein Targeting Ligand binds a protein that is upregulated in amyloidosis. In some embodiments, the amyloidosis can be systemic amyloidosis. In other embodiments, the amyloidosis can be localized amyloidosis. In some embodiments, the protein upregulated in systemic amyloidosis can be transthyretin.

In some embodiments, the Extracellular Protein Targeting Ligand binds an immune checkpoint protein. In some embodiments, the target protein comprises a cancer antigen. In certain embodiments, the cancer antigen comprises HER2, EGFR, CDCP1, CD38, IGF-1R, MMP14, and TROP2.

In some embodiments, the Extracellular Protein Targeting Ligand binds an immunomodulatory protein. In certain embodiments, the immunomodulatory protein comprises PD-L1, PD-1, CTLA-4, B7-H3, B7-H4, LAG3, NKG2D, TIM-3, VISTA, CD39, CD73 (NT5E), A2AR, SIGLEC7, and SIGLEC15. In some embodiments, the target protein comprises a B cell antigen. In some exemplary embodiments, the B cell antigen comprises CD 19 and CD20.

In certain embodiments, the Extracellular Protein Targeting Ligand binds a soluble target protein. In some embodiments, the soluble target protein comprises an inflammatory cytokine, a growth factor (GF), a toxic enzyme, a target associated with metabolic diseases, a neuronal aggregate, or an autoantibody. In certain embodiments, the inflammatory cytokine comprises lymphotoxin, interleukin-1 (IL-1), IL-2, IL-5, IL-6, IL-12, IL-13, IL-17, IL-18, IL-23, tumor necrosis factor alpha (TNF-α), interferon gamma (IFNy), and granulocyte-macrophage colony stimulating factor (GM-CSF). In certain embodiments, the growth factor comprises EGF, FGF, NGF, PDGF, VEGF, IGF, GMCSF, GCSF, TGF, RANK-L, erythropieitn, TPO, BMP, HGF, GDF, neurotrophins, MSF, SGF, GDF, and an isoform thereof. In certain embodiments, the toxic enzyme comprises a protein arginine deiminase 1 (PAD1), PAD2, PAD3, PAD4, and PAD6, leucocidin, hemolysin, coagulase, treptokinase, hyaluronidase. In certain embodiments, the toxic enzyme comprises PAD2 or PAD4. In some embodiments, the neuronal aggregate comprises Ab, TTR, a-synuclein, TAO, and prion. In certain embodiments, the autoantibody comprises IgA, IgE, IgG, IgM and IgD.

In certain embodiments, the Extracellular Protein Targeting Ligand binds a growth factor, a cytokine, a chemokine, a hormone, a neurotransmitter, a capsid, a soluble receptor, an extracellular secreted protein, an antibody, a lipoprotein, an exosome, a virus, a cell, or a plasma membrane protein, wherein the bifunctional compounds of the invention can then be used to direct the extracellular target molecule to lysosomes for degradation. Examples of such target molecules which can be directed for degradation using the bifunctional compound of the invention include, but are not limited to, LDL (ApoB), Lp(a), ApoCIII, ANGPTL3, ANGPTL4, ANGPTL8, Factor 11, GDF15, LPL, PCSK9, IL1 J, IL17, Complement Factor B, Complement Factor D, MPO, IgE, IL7, IL12A, IL23, TNFA, CXCR4, MAPT, FHR3, TIMP1, Apelin, BMP6, BMP9/GDF2, CSF-1, EPO, IL5, MFGE8, TSLP, TSP, C5, CXCL10, FGF23, IGF1, IL10, IL13, IL2, IL6, VEGFA, NKG2D, ZNFR3, ADA2, suPAR, TGF-β1, IL4 receptor, sToll receptor, histamine, Tau, proglanulin, Alpha-synuclein, toxins, venoms, HBV soluble antigen, viral antigens, prion protein, scFV, AAV and anti-AAV antibodies. In certain embodiments, the extracellular target molecules which can be directed for degradation using the bifunctional compound of the invention are PCSK9 and FHR3.

In certain embodiments, the Extracellular Protein Targeting Ligand binds a protein selected from proprotein convertase subtilisin/kexin type 9 (PCSK9), tumor necrosis factor receptor 1 (TNFR1), interleukin-1 receptor (IL1R), low density lipoproteins, very low density lipoproteins, chylomicrons, apolipoprotein B (ApoB), lipoprotein(a) (Lp(a)), apolipoprotein C3 (ApoCIII), angiopoietin-like 3 (ANGPTL3), angiopoietin-like 4 (ANGPTL4), angiopoietin-like 8 (ANGPTL8), Factor 11, growth differentiation factor 15 (GDF15), lipoprotein lipase (LPL), interleukin 1-beta (IL 113), interleukin 17 (IL 17), complement Factor B, complement Factor D, myeloperoxidase (MPO), immunoglobulin A (IgA), immunoglobulin E (IgE), programmed cell death protein 1 (PD-1), programmed death-ligand 1 (PD-L1), interleukin 7 (IL7), interleukin 12A (IL12A), interleukin 23 (IL23), tumor necrosis factor A (TNFA), microtubule associated protein tau (MAPT), complement factor H-related protein 3 (FHR3), tissue inhibitor of metalloproteinases 1 (TIMP1), Apelin, bone morphogenetic protein 6 (BMP6), bone morphogenetic protein 9/growth differentiation factor 2 (BMP9/GDF2), colony stimulating factor 1 receptor (CSF-1), erythropoietin (EPO), interleukin 5 (IL5), milk fat globule-EGF Factor 8 protein (MFGE8), thymic stromal lymphopoietin (TSLP), thrombospondin (TSP), complement component 5 (C5), C-X-C motif chemokine ligand 10 (CXCL10), fibroblast growth factor 23 (FGF23), insulin-like growth factor 1 (IGF1), interleukin 10 (IL 10), interleukin 13 (IL 13), interleukin 2 (IL2), interleukin 6 (IL6), vascular endothelial growth factor A (VEGF-A), adenosine deaminase 2 (ADA2), soluble urokinase-type plasminogen activator receptor (suPAR), transforming growth factor beta 1 (TGF-pi), progranulin, alpha-synuclein, a toxin, a venom, an HBV soluble antigen, a viral antigen, a prion protein, a scFv, an AAV, and an anti-AAV antibody.

### Embodiments of the Linker

In non-limiting embodiments, Linker^{A} and Linker^{B} are independently selected from:

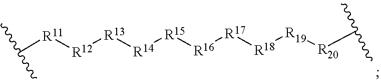

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR'S(O)R³, haloalkyl, aryl, heteroaryl, and heterocycle;
and the remaining variables are as defined herein.

In certain embodiments Linker^{A} is bond and Linker^{B} is

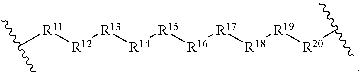

In certain embodiments Linker^{B} is bond and Linker^{A} is

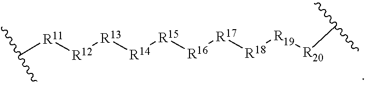

In certain embodiments, R¹¹, R¹², R¹³, R¹⁴, R¹⁵, R¹⁶, R¹⁷, R¹⁸, R¹⁹, and R²⁰ are bond. In certain embodiments, R¹², R¹³, R¹⁴, R¹⁵, R¹⁶, R¹⁷, R¹⁸, R¹⁹, and R²⁰ are bond. In certain embodiments, R¹³, R¹⁴, R¹⁵, R¹⁶, R¹⁷, R¹⁸, R¹⁹, and R²⁰ are bond. In certain embodiments, R¹⁴, R¹⁵, R¹⁶, R¹⁷, R¹⁸, R¹⁹, and R²⁰ are bond. In certain embodiments, R¹⁵, R¹⁶, R¹⁷, R¹⁸, R¹⁹, and R²⁰ are bond. In certain embodiments, R¹⁶, R¹⁷, R¹⁸, R¹⁹, and R²⁰ are bond. In certain embodiments, R¹⁷, R¹⁸, R¹⁹, and R²⁰ are bond. In certain embodiments, R¹⁸, R¹⁹, and R²⁰ are bond.

In certain embodiments, nine of R¹¹, R¹², R¹³, R¹⁴, R¹⁵, R¹⁶, R¹⁷, R¹⁸, R¹⁹, and R²⁰ are bond. In certain embodiments, eight of R¹¹, R¹², R¹³, R¹⁴, R¹⁵, R¹⁶, R¹⁷, R¹⁸, R¹⁹, and R²⁰ are bond. In certain embodiments, seven of R¹¹, R¹², R¹³, R¹⁴, R¹⁵, R¹⁶, R¹⁷, R¹⁸, R¹⁹, and R²⁰ are bond. In certain embodiments, six of R¹¹, R¹², R¹³, R¹⁴, R¹⁵, R¹⁶, R¹⁷, R¹⁸, R¹⁹, and R²⁰ are bond. In certain embodiments, five of R¹¹, R¹², R¹³, R¹⁴, R¹⁵, R¹⁶, R¹⁷, R¹⁸, R¹⁹, and R²⁰ are bond. In certain embodiments, four of R¹¹, R¹², R¹³, R¹⁴, R¹⁵, R¹⁶, R¹⁷, R¹⁸, R¹⁹, and R²⁰ are bond. In certain embodiments, three of R¹¹, R¹², R¹³, R¹⁴, R¹⁵, R¹⁶, R¹⁷, R¹⁸, R¹⁹, and R²⁰ are bond. In certain embodiments, two of R¹¹, R¹², R¹³, R¹⁴, R¹⁵, R¹⁶, R¹⁷, R¹⁸, R¹⁹, and R²⁰ are bond. In certain embodiments, one of R¹¹, R¹², R¹³, R¹⁴, R¹⁵, R¹⁶, R¹⁷, R¹⁸, R¹⁹, and R²⁰ is bond.

In certain embodiments, Linker^{A} is

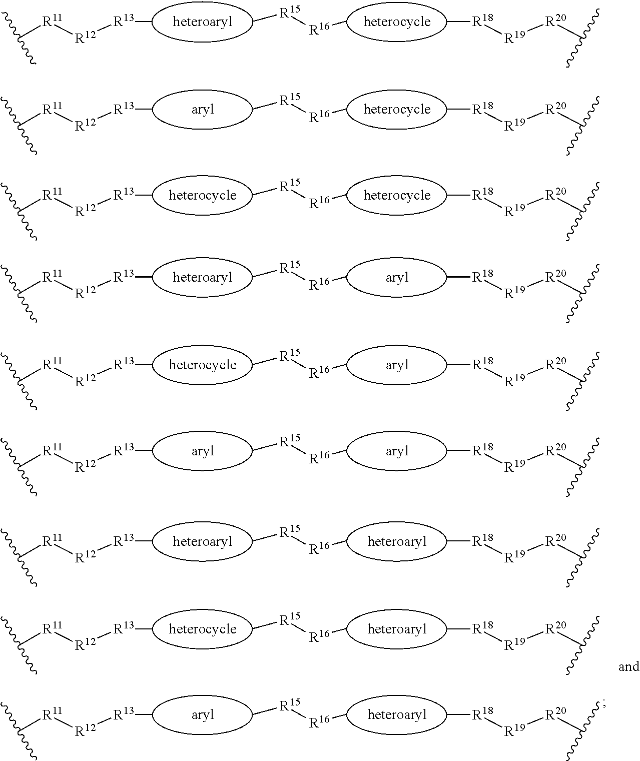

wherein each heteroaryl, heterocycle, and aryl can optionally be substituted with 1, 2, 3, or 4 of any combination of halogen, alkyl, haloalkyl, aryl, heteroaryl, heterocycle, or cycloalkyl.

In certain embodiments, R¹¹, R¹², R¹³, R¹⁵, R¹⁶, R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.

In certain embodiments, Linker^{A} is selected from

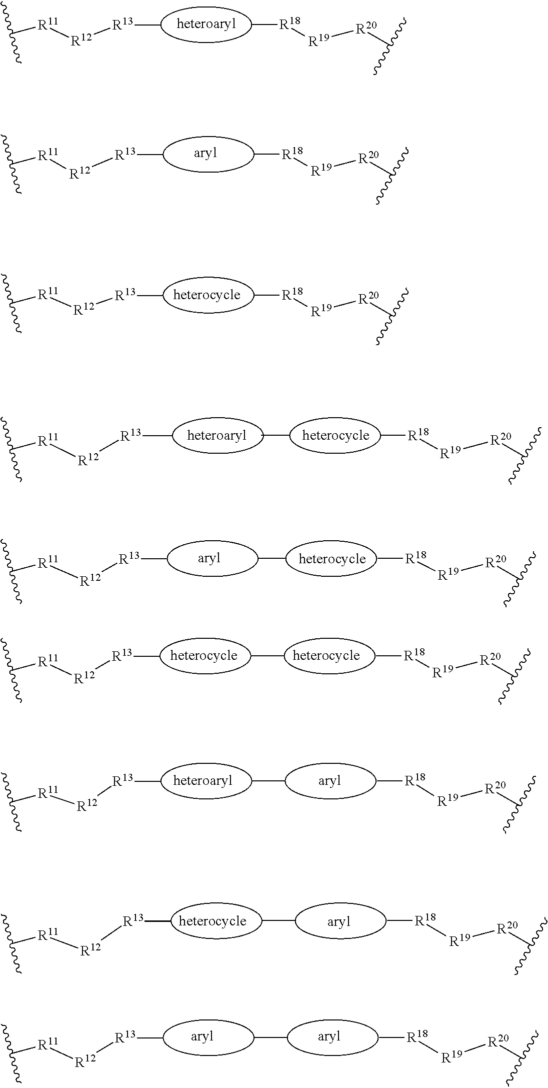

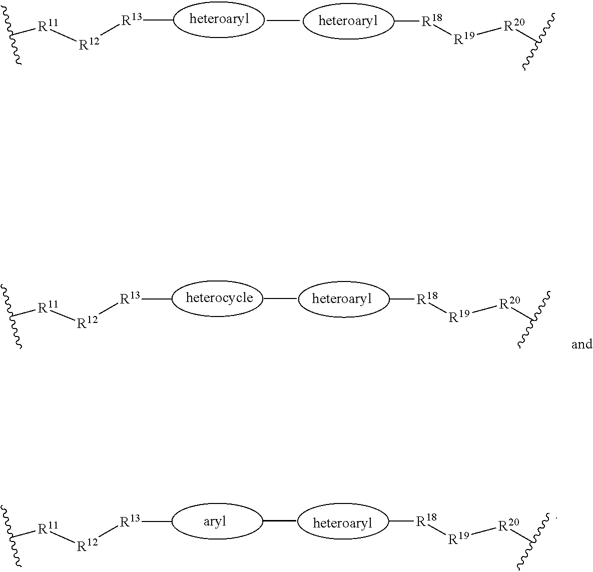

In certain embodiments, R¹¹, R¹², R¹³, R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.

In certain embodiments, R¹¹ is selected from the group consisting of bond, CH₂, —O—, —C(O)NR⁶— and —C(O)O—. In certain embodiments, R²⁰ is selected from the group consisting of bond, CH₂, —O—, —C(O)NR⁶— and —C(O)O—.

In certain embodiments, Linker^{A} is selected from

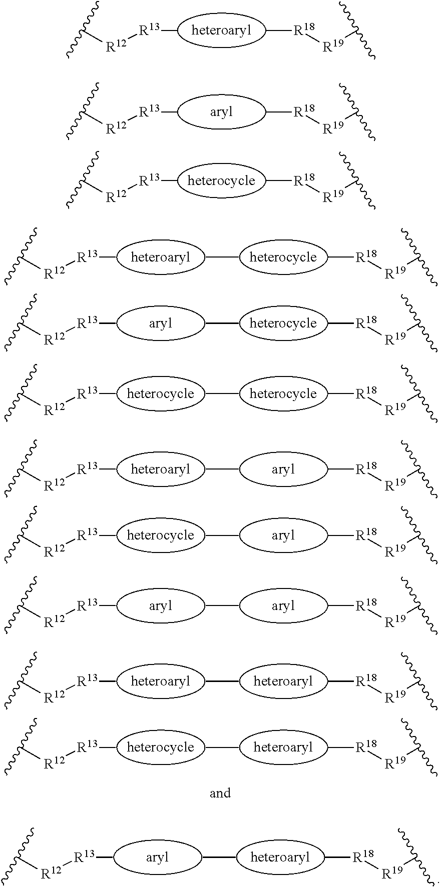

In certain embodiments, R¹² is selected from the group consisting of bond, CH₂, —O—, —C(O)NR⁶— and —C(O)O—. In certain embodiments, R¹⁹ is selected from the group consisting of bond, CH₂, —O—, —C(O)NR⁶— and —C(O)O—.

In certain embodiments, Linker^{A} is selected from

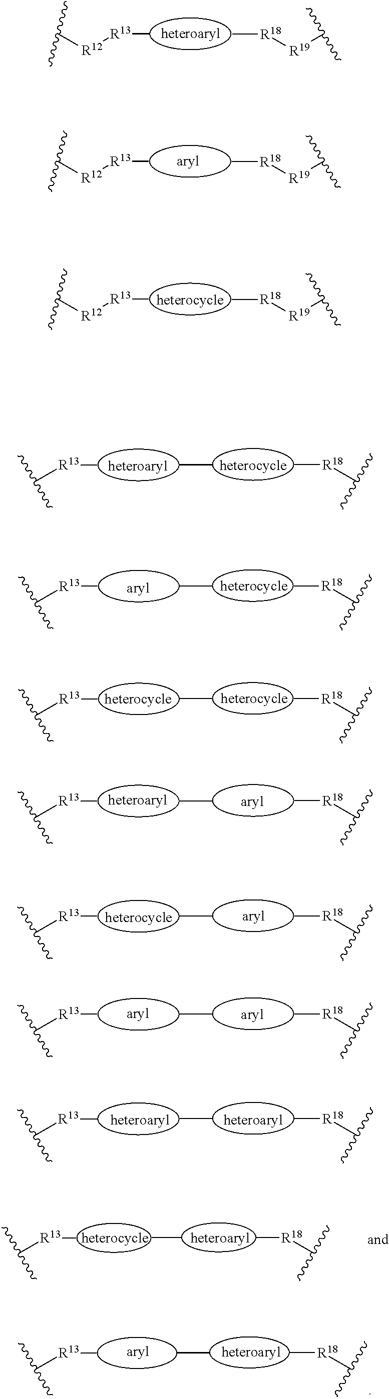

In certain embodiments, R¹³ is selected from the group consisting of bond, CH₂, —O—, —C(O)NR⁶— and —C(O)O—. In certain embodiments, R¹⁸ is selected from the group consisting of bond, CH₂, —O—, —C(O)NR⁶— and —C(O)O—.

In certain embodiments, aryl is phenyl.

In certain embodiments, heteroaryl is selected from

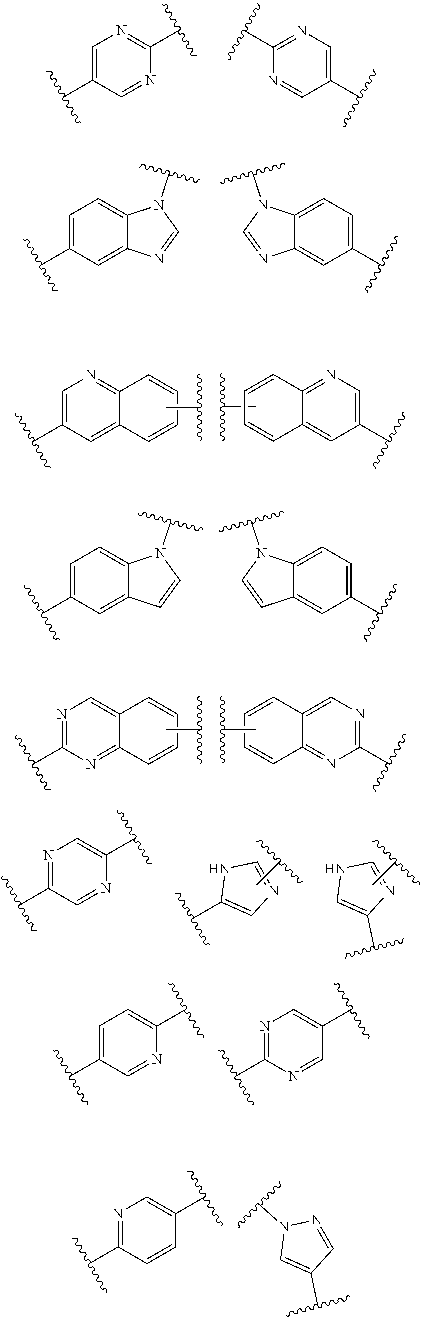

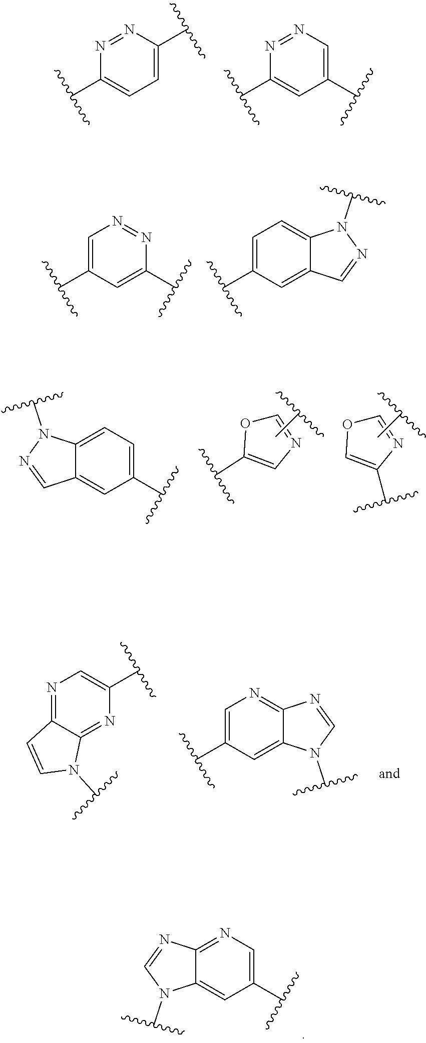

In certain embodiments, heteroaryl is selected from

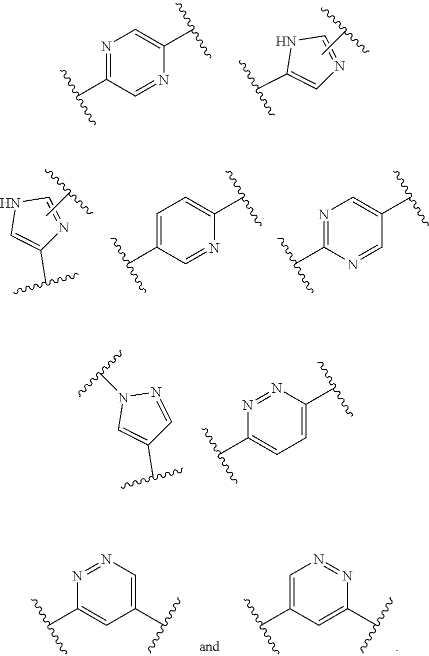

In certain embodiments, heterocycle is selected from R

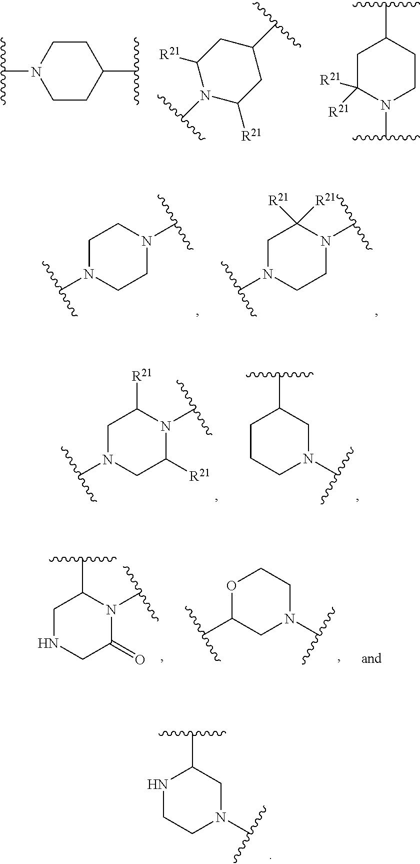

In certain embodiments, Linker^{A} is selected from

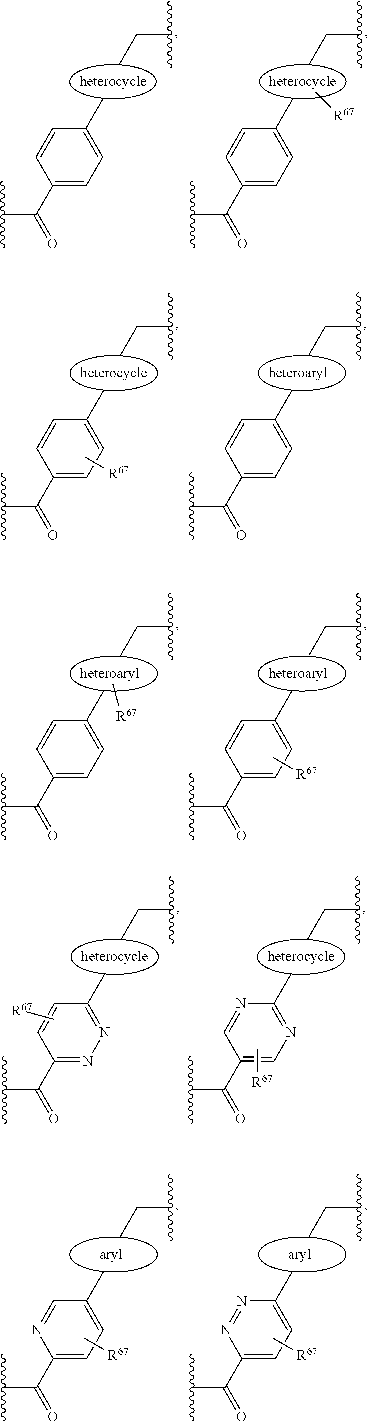

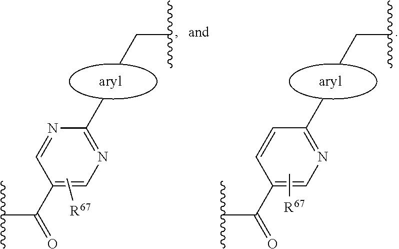

In certain embodiments, Linker^{A} is selected from

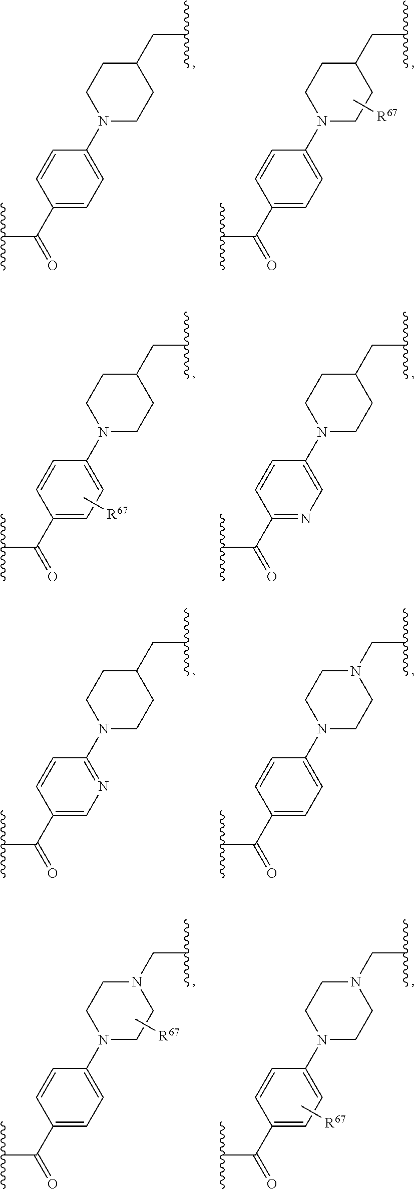

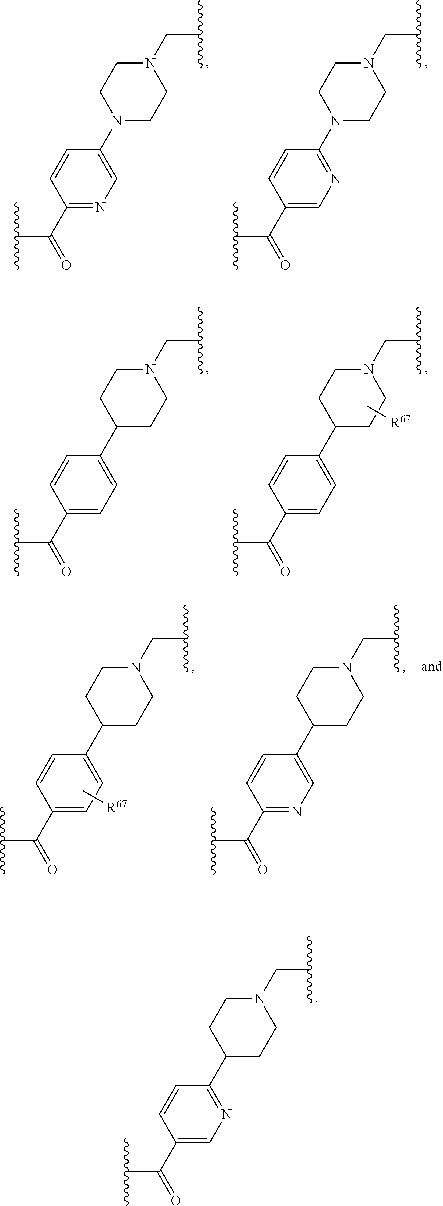

In certain embodiments, Linker^{B} is:

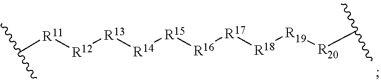

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, alkenyl, alkynyl, haloalkyl, alkoxy, aryl, heterocycle, heteroaryl, —CH₂CH₂—[O—(CH₂)₂]ₙ—O—, and —CH₂CH₂—[O—(CH₂)₂]ₙ—NR⁶—; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.

In certain embodiments of Linker^{B}, R¹¹, R¹², R¹³, R¹⁴, R¹⁵, R¹⁶, and R¹⁷ are bond. In certain embodiments of Linker^{B}, five of R¹¹, R¹², R¹³, R¹⁴, R¹⁵, R¹⁶, R¹⁷, R¹⁸, and R¹⁹ are bond. In certain embodiments of Linker^{B}, four of R¹¹, R¹², R¹³, R¹⁴, R¹⁵, R¹⁶, R¹⁷, R¹⁸, and R¹⁹ are bond. In certain embodiments of Linker^{B}, three of R¹¹, R¹², R¹³, R¹⁴, R¹⁵, R¹⁶, R¹⁷, R¹⁸, and R¹⁹ are bond. In certain embodiments of Linker^{B}, R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.

### Additional Embodiments of Linker^{A}

1. In certain embodiments of Linker^{A}, R¹¹ is bond.
2. In certain embodiments of Linker^{A}, R¹¹ is alkyl.
3. In certain embodiments of Linker^{A}, R¹¹ is —C(O)—.
4. In certain embodiments of Linker^{A}, R¹¹ is —C(O)O—.
5. In certain embodiments of Linker^{A}, R¹¹ is —C(O)NR⁶—.
6. In certain embodiments of Linker^{A}, R¹¹ is —NR⁶C(O)—.
7. In certain embodiments of Linker^{A}, R¹¹ is —NR⁶—.
8. In certain embodiments of Linker^{A}, R¹¹ is —O—.
9. In certain embodiments of Linker^{A}, R¹¹ is —C(R²¹R²¹)—.
10. The Linker^{A} of embodiment 12, wherein both R²¹ groups are hydrogen.
11. In certain embodiments of Linker^{A}, R¹¹ is —CH₂CH₂—[O—(CH₂)₂]ₙ—O—.
12. In certain embodiments of Linker^{A}, R¹¹ is aryl.
13. The Linker^{A} of embodiment 12, wherein R¹¹ is phenyl.
14. The Linker^{A} of embodiment 12, wherein R¹¹ is naphthyl.
15. In certain embodiments of Linker^{A}, R¹¹ is heterocycle.
16. The Linker^{A} of embodiment 15, wherein R¹¹ is piperidinyl.
17. The Linker^{A} of embodiment 16, wherein R¹¹ is

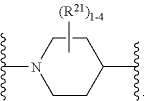

18. The Linker^{A} of embodiment 16, wherein R¹¹ is

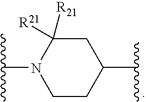

19. The Linker^{A} of embodiment 15, wherein R¹¹ is piperizinyl.
20. The Linker^{A} of embodiment 19, wherein R¹¹ is

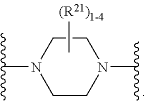

21. The Linker^{A} of embodiment 15, wherein R¹¹ is pyrrolidinyl.
22. The Linker^{A} of embodiment 21, wherein R¹¹ is

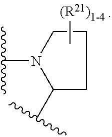

23. The Linker^{A} of embodiment 21, wherein R¹¹ is

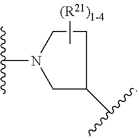

24. In certain embodiments of Linker^{A}, R¹¹ is heteroaryl.
25. The Linker^{A} of embodiment 24, wherein R¹¹ is pyridinyl.
26. The Linker^{A} of embodiment 25, wherein R¹¹ is

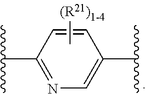

27. The Linker^{A} of embodiment 25, wherein R¹¹ is

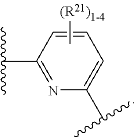

28. The Linker^{A} of embodiment 25, wherein R¹¹ is

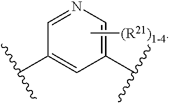

29. The Linker^{A} of embodiment 24, wherein R¹¹ is pyrimidinyl.
30. The Linker^{A} of embodiment 29, wherein R¹¹ is

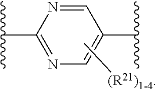

31. The Linker^{A} of embodiment 29, wherein R¹¹ is

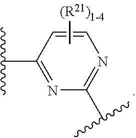

32. The Linker^{A} of embodiment 24, wherein R¹¹ is pyrazinyl.
33. The Linker^{A} of embodiment 32, wherein R¹¹ is

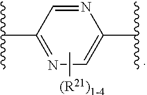

34. The Linker^{A} of embodiment 32, wherein R¹¹ is

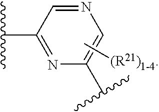

35. The Linker^{A} of embodiment 24, wherein R¹¹ is pyridizinyl.
36. The Linker^{A} of embodiment 35, wherein R¹¹ is

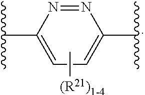

37. The Linker^{A} of embodiment 35, wherein R¹¹ is

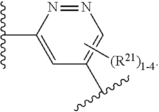

38. In certain embodiments of Linker^{A}, R¹¹ is alkynyl.
39. In certain embodiments of Linker^{A}, R¹¹ is alkoxy.
40. The Linker^{A} of any one of embodiments 1-39, wherein R¹² is bond.
41. The Linker^{A} of any one of embodiments 1-39, wherein R¹² is alkyl.
42. The Linker^{A} of any one of embodiments 1-39, wherein R¹² is —C(O)—.
43. The Linker^{A} of any one of embodiments 1-39, wherein R¹² is —C(O)O—.
44. The Linker^{A} of any one of embodiments 1-39, wherein R¹² is —C(O)NR⁶—.
45. The Linker^{A} of any one of embodiments 1-39, wherein R¹² is —NR⁶C(O)—.
46. The Linker^{A} of any one of embodiments 1-39, wherein R¹² is —C(R²¹R²¹)—.
47. The Linker^{A} of embodiment 46, wherein both R²¹ groups are hydrogen.
48. The Linker^{A} of any one of embodiments 1-39, wherein R¹² is —CH₂CH₂—[O—(CH₂)₂]ₙ—O—.
49. The Linker^{A} of any one of embodiments 1-39, wherein R¹² is aryl.
50. The Linker^{A} of embodiment 39, wherein R¹² is phenyl.
51. The Linker^{A} of embodiment 39, wherein R¹² is naphthyl.
52. The Linker^{A} of any one of embodiments 1-39, wherein R¹² is heterocycle.
53. The Linker^{A} of embodiment 52, wherein R¹² is piperidinyl.
54. The Linker^{A} of embodiment 53, wherein R¹² is

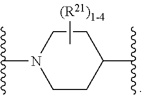

55. The Linker^{A} of embodiment 53, wherein R¹² is

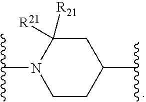

56. The Linker^{A} of embodiment 52, wherein R¹² is piperizinyl.
57. The Linker^{A} of embodiment 56, wherein R¹² is

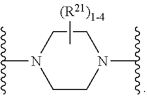

58. The Linker^{A} of embodiment 52, wherein R¹² is pyrrolidinyl.
59. The Linker^{A} of embodiment 58, wherein R¹² is

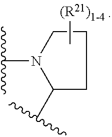

60. The Linker^{A} of embodiment 59, wherein R¹² is

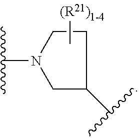

61. The Linker^{A} of any one of embodiments 1-39, wherein R¹² is heteroaryl.
62. The Linker^{A} of embodiment 61, wherein R¹² is pyridinyl.
63. The Linker^{A} of embodiment 62, wherein R¹² is

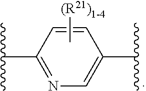

64. The Linker^{A} of embodiment 62, wherein R¹² is

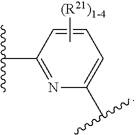

65. The Linker^{A} of embodiment 62, wherein R¹² is

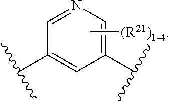

66. The Linker^{A} of embodiment 61, wherein R¹² is pyrimidinyl.
67. The Linker^{A} of embodiment 66, wherein R¹² is

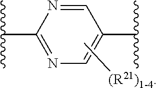

68. The Linker^{A} of embodiment 66, wherein R¹² is

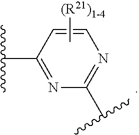

69. The Linker^{A} of embodiment 61, wherein R¹² is pyrazinyl.
70. The Linker^{A} of embodiment 69, wherein R¹² is

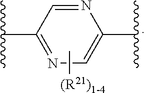

71. The Linker^{A} of embodiment 68, wherein R¹² is

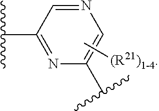

72. The Linker^{A} of embodiment 61, wherein R¹² is pyridizinyl.
73. The Linker^{A} of embodiment 72, wherein R¹² is

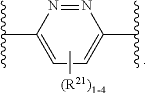

74. The Linker^{A} of embodiment 72, wherein R¹² is

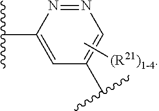

75. The Linker^{A} of any one of embodiments 1-39, wherein R¹² is alkynyl.
76. The Linker^{A} of any one of embodiments 1-39, wherein R¹² is alkoxy.
77. The Linker^{A} of any one of embodiments 1-76, wherein R¹³ is bond.
78. The Linker^{A} of any one of embodiments 1-76, wherein R¹³ is alkyl.
79. The Linker^{A} of any one of embodiments 1-76, wherein R¹³ is —C(O)—.
80. The Linker^{A} of any one of embodiments 1-76, wherein R¹³ is —C(O)O—.
81. The Linker^{A} of any one of embodiments 1-76, wherein R¹³ is —C(O)NR⁶—.
82. The Linker^{A} of any one of embodiments 1-76, wherein R¹³ is —NR⁶C(O)—.
83. The Linker^{A} of any one of embodiments 1-76, wherein R¹³ is —NR⁶—.
84. The Linker^{A} of any one of embodiments 1-76, wherein R¹³ is —O—.
85. The Linker^{A} of any one of embodiments 1-76, wherein R¹³ is —C(R²¹R²¹)—.
86. The Linker^{A} of embodiment 85, wherein both R²¹ groups are hydrogen.
87. The Linker^{A} of any one of embodiments 1-76, wherein R¹³ is —CH₂CH₂—[O—(CH₂)₂]ₙ—O—.
88. The Linker^{A} of any one of embodiments 1-76, wherein R¹³ is aryl.
89. The Linker^{A} of embodiment 88, wherein R¹³ is phenyl.
90. The Linker^{A} of embodiment 88, wherein R¹³ is naphthyl.
91. The Linker^{A} of any one of embodiments 1-76, wherein R¹³ is heterocycle.
92. The Linker^{A} of embodiment 91, wherein R¹³ is piperidinyl.
93. The Linker^{A} of embodiment 92, wherein R¹³ is

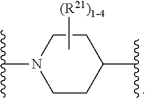

94. The Linker^{A} of embodiment 92, wherein R¹³ is

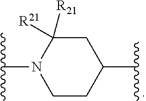

95. The Linker^{A} of embodiment 91, wherein R¹³ is piperizinyl.
96. The Linker^{A} of embodiment 95, wherein R¹³ is

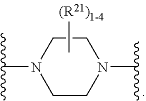

97. The Linker^{A} of embodiment 91, wherein R¹³ is pyrrolidinyl.
98. The Linker^{A} of embodiment 97, wherein R¹³ is

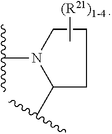

99. The Linker^{A} of embodiment 97, wherein R¹³ is

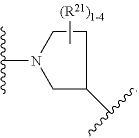

100. The Linker^{A} of any one of embodiments 1-76, wherein R¹³ is heteroaryl.
101. The Linker^{A} of embodiment 100, wherein R¹³ is pyridinyl.
102. The Linker^{A} of embodiment 101, wherein R¹³ is

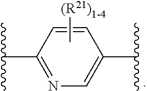

103. The Linker^{A} of embodiment 101, wherein R¹³ is

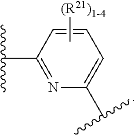

104. The Linker^{A} of embodiment 101, wherein R¹³ is

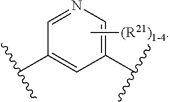

105. The Linker^{A} of embodiment 100, wherein R¹³ is pyrimidinyl.
106. The Linker^{A} of embodiment 105, wherein R¹³ is

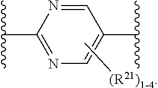

107. The Linker^{A} of embodiment 105, wherein R¹³ is

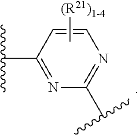

108. The Linker^{A} of embodiment 100, wherein R¹³ is pyrazinyl.
109. The Linker^{A} of embodiment 108, wherein R¹³ is

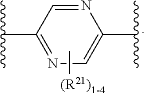

110. The Linker^{A} of embodiment 108, wherein R¹³ is

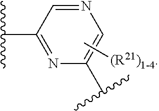

111. The Linker^{A} of embodiment 100, wherein R¹³ is pyridizinyl.
112. The Linker^{A} of embodiment 111, wherein R³ is

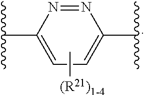

113. The Linker^{A} of embodiment 111, wherein R¹³ is

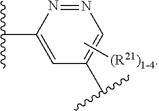

114. The Linker^{A} of any one of embodiments 1-76, wherein R¹³ is alkynyl.
115. The Linker^{A} of any one of embodiments 1-76, wherein R¹³ is alkoxy.
116. The Linker^{A} of any one of embodiments 1-115, wherein R¹⁴ is bond.
117. The Linker^{A} of any one of embodiments 1-115, wherein R¹⁴ is alkyl.
118. The Linker^{A} of any one of embodiments 1-115, wherein R¹⁴ is —C(O)—.
119. The Linker^{A} of any one of embodiments 1-115, wherein R¹⁴ is —C(O)O—.
120. The Linker^{A} of any one of embodiments 1-115, wherein R¹⁴ is —C(O)NR⁶—.
121. The Linker^{A} of any one of embodiments 1-115, wherein R¹⁴ is —NR⁶C(O)—.
122. The Linker^{A} of any one of embodiments 1-115, wherein R¹⁴ is —S(O)—.
123. The Linker^{A} of any one of embodiments 1-115, wherein R¹⁴ is —S(O)₂—.
124. The Linker^{A} of any one of embodiments 1-115, wherein R¹⁴ is —C(R²¹R²¹)—.
125. The Linker^{A} of embodiment 124, wherein both R²¹ groups are hydrogen.
126. The Linker^{A} of any one of embodiments 1-115, wherein R¹⁴ is —CH₂CH₂—[O—(CH₂)₂]ₙ—O—.
127. The Linker^{A} of any one of embodiments 1-115, wherein R¹⁴ is aryl.
128. The Linker^{A} of embodiment 127, wherein R¹⁴ is phenyl.
129. The Linker^{A} of embodiment 127, wherein R¹⁴ is naphthyl.
130. The Linker^{A} of any one of embodiments 1-115, wherein R¹⁴ is heterocycle.
131. The Linker^{A} of embodiment 130, wherein R¹⁴ is piperidinyl.
132. The Linker^{A} of embodiment 131, wherein R¹⁴ is

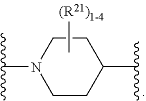

133. The Linker^{A} of embodiment 131, wherein R¹⁴ is

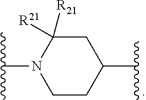

134. The Linker^{A} of embodiment 130, wherein R¹⁴ is piperizinyl.
135. The Linker^{A} of embodiment 134, wherein R¹⁴ is

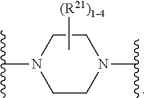

136. The Linker^{A} of embodiment 130, wherein R¹⁴ is pyrrolidinyl.
137. The Linker^{A} of embodiment 136, wherein R¹⁴ is

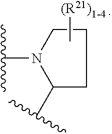

138. The Linker^{A} of embodiment 136, wherein R¹⁴ is

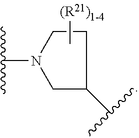

139. The Linker^{A} of any one of embodiments 1-115, wherein R¹⁴ is heteroaryl.
140. The Linker^{A} of embodiment 139, wherein R¹⁴ is pyridinyl.
141. The Linker^{A} of embodiment 140, wherein R¹⁴ is

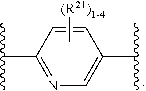

142. The Linker^{A} of embodiment 140, wherein R¹⁴ is

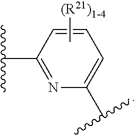

143. The Linker^{A} of embodiment 140, wherein R¹⁴ is

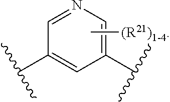

144. The Linker^{A} of embodiment 139, wherein R¹⁴ is pyrimidinyl.
145. The Linker^{A} of embodiment 144, wherein R¹⁴ is

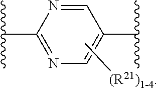

146. The Linker^{A} of embodiment 144, wherein R¹⁴ is

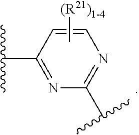

147. The Linker^{A} of embodiment 139, wherein R¹⁴ is pyrazinyl.
148. The Linker^{A} of embodiment 147, wherein R¹⁴ is

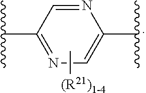

149. The Linker^{A} of embodiment 147, wherein R¹⁴ is

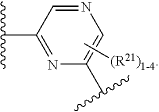

150. The Linker^{A} of embodiment 139, wherein R¹⁴ is pyridizinyl.
151. The Linker^{A} of embodiment 150, wherein R¹⁴ is

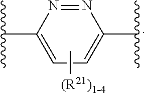

152. The Linker^{A} of embodiment 150, wherein R¹⁴ is

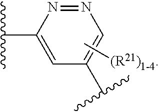

153. The Linker^{A} of any one of embodiments 1-115, wherein R⁴ is alkynyl.
154. The Linker^{A} of any one of embodiments 1-115, wherein R¹⁴ is alkoxy.
155. The Linker^{A} of any one of embodiments 1-154, wherein R¹⁵ is bond.
156. The Linker^{A} of any one of embodiments 1-154, wherein R¹⁵ is alkyl.
157. The Linker^{A} of any one of embodiments 1-154, wherein R¹⁵ is —C(O)—.
158. The Linker^{A} of any one of embodiments 1-154, wherein R¹⁵ is —C(O)O—.
159. The Linker^{A} of any one of embodiments 1-154, wherein R¹⁵ is —C(O)NR⁶—.
160. The Linker^{A} of any one of embodiments 1-154, wherein R¹⁵ is —NR⁶C(O)—.
161. The Linker^{A} of any one of embodiments 1-154, wherein R¹⁴ is —NR⁶—.
162. The Linker^{A} of any one of embodiments 1-154, wherein R¹⁴ is —O—.
163. The Linker^{A} of any one of embodiments 1-154, wherein R¹⁴ is —C(R²¹R²¹)—.
164. The Linker^{A} of embodiment 163, wherein both R²¹ groups are hydrogen.
165. The Linker^{A} of any one of embodiments 1-154, wherein R¹⁵ is —CH₂CH₂—[O—(CH₂)₂]ₙ—O—.
166. The Linker^{A} of any one of embodiments 1-154, wherein R¹⁵ is aryl.
167. The Linker^{A} of embodiment 166, wherein R¹⁵ is phenyl.
168. The Linker^{A} of embodiment 166, wherein R¹⁵ is naphthyl.
169. The Linker^{A} of any one of embodiments 1-154, wherein R¹⁵ is heterocycle.
170. The Linker^{A} of embodiment 169, wherein R¹⁵ is piperidinyl.
171. The Linker^{A} of embodiment 170, wherein R⁵ is

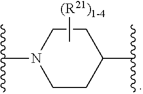

172. The Linker^{A} of embodiment 170, wherein R¹⁵ is

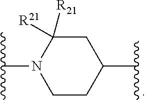

173. The Linker^{A} of embodiment 169, wherein R¹⁵ is piperizinyl.
174. The Linker^{A} of embodiment 173, wherein R¹⁵ is

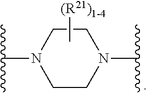

175. The Linker^{A} of embodiment 169, wherein R¹⁵ is pyrrolidinyl.
176. The Linker^{A} of embodiment 175, wherein R¹⁵ is

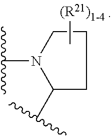

177. The Linker^{A} of embodiment 175, wherein R¹⁵ is

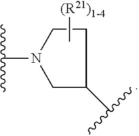

178. The Linker^{A} of any one of embodiments 1-154, wherein R¹⁵ is heteroaryl.
179. The Linker^{A} of embodiment 178, wherein R¹⁵ is pyridinyl.
180. The Linker^{A} of embodiment 179, wherein R¹⁵ is

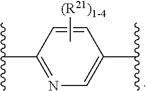

181. The Linker^{A} of embodiment 179, wherein R¹⁵ is

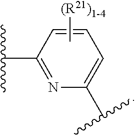

182. The Linker^{A} of embodiment 179, wherein R¹⁵ is

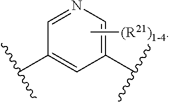

183. The Linker^{A} of embodiment 178, wherein R¹⁵ is pyrimidinyl.
184. The Linker^{A} of embodiment 183, wherein R¹⁵ is

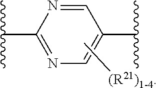

185. The Linker^{A} of embodiment 183, wherein R¹⁵ is

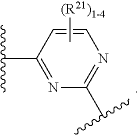

186. The Linker^{A} of embodiment 178, wherein R¹⁵ is pyrazinyl.
187. The Linker^{A} of embodiment 186, wherein R¹⁵ is

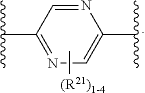

188. The Linker^{A} of embodiment 186, wherein R¹⁵ is

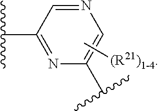

189. The Linker^{A} of embodiment 178, wherein R¹⁵ is pyridizinyl.
190. The Linker^{A} of embodiment 189, wherein R¹⁵ is

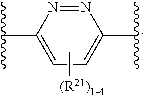

191. The Linker^{A} of embodiment 189, wherein R¹⁵ is

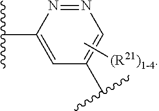

192. The Linker^{A} of any one of embodiments 1-154, wherein R¹⁵ is alkynyl.
193. The Linker^{A} of any one of embodiments 1-154, wherein R¹⁵ is alkoxy.
194. The Linker^{A} of any one of embodiments 1-193, wherein R¹⁶ is bond.
195. The Linker^{A} of any one of embodiments 1-193, wherein R¹⁶ is alkyl.
196. The Linker^{A} of any one of embodiments 1-193, wherein R¹⁶ is —C(O)—.
197. The Linker^{A} of any one of embodiments 1-193, wherein R¹⁶ is —C(O)O—.
198. The Linker^{A} of any one of embodiments 1-193, wherein R¹⁶ is —C(O)NR⁶—.
199. The Linker^{A} of any one of embodiments 1-193, wherein R¹⁶ is —NR⁶C(O)—.
200. The Linker^{A} of any one of embodiments 1-193, wherein R¹⁶ is —S(O)—.
201. The Linker^{A} of any one of embodiments 1-193, wherein R¹⁶ is —S(O)₂—.
202. The Linker^{A} of any one of embodiments 1-193, wherein R¹⁶ is —C(R²¹R²¹)—.
203. The Linker^{A} of embodiment 202, wherein both R²¹ groups are hydrogen.
204. The Linker^{A} of any one of embodiments 1-193, wherein R¹⁶ is —CH₂CH₂—[O—(CH₂)₂]ₙ—O—.
205. The Linker^{A} of any one of embodiments 1-193, wherein R¹⁶ is aryl.
206. The Linker^{A} of embodiment 205, wherein R¹⁶ is phenyl.
207. The Linker^{A} of embodiment 205, wherein R¹⁶ is naphthyl.
208. The Linker^{A} of any one of embodiments 1-193, wherein R¹⁶ is heterocycle.
209. The Linker^{A} of embodiment 208, wherein R¹⁶ is piperidinyl.
210. The Linker^{A} of embodiment 209, wherein R¹⁶ is

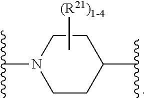

211. The Linker^{A} of embodiment 209, wherein R¹⁶ is

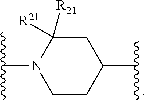

212. The Linker^{A} of embodiment 208, wherein R¹⁶ is piperizinyl.
213. The Linker^{A} of embodiment 212, wherein R¹⁶ is

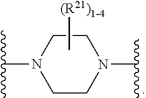

214. The Linker^{A} of embodiment 208, wherein R¹⁶ is pyrrolidinyl.
215. The Linker^{A} of embodiment 214, wherein R¹⁶ is

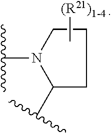

216. The Linker^{A} of embodiment 214, wherein R¹⁶ is

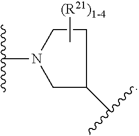

217. The Linker^{A} of any one of embodiments 1-193, wherein R¹⁶ is heteroaryl.
218. The Linker^{A} of embodiment 217, wherein R¹⁶ is pyridinyl.
219. The Linker^{A} of embodiment 218, wherein R¹⁶ is

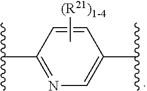

220. The Linker^{A} of embodiment 218, wherein R¹⁶ is

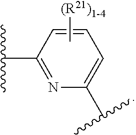

221. The Linker^{A} of embodiment 218, wherein R¹⁶ is

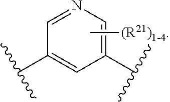

222. The Linker^{A} of embodiment 217, wherein R¹⁶ is pyrimidinyl.
223. The Linker^{A} of embodiment 222, wherein R¹⁶ is

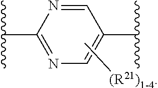

224. The Linker^{A} of embodiment 222, wherein R¹⁶ is

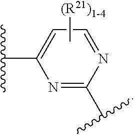

225. The Linker^{A} of embodiment 217, wherein R¹⁶ is pyrazinyl.
226. The Linker^{A} of embodiment 225, wherein R¹⁶ is

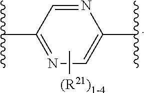

227. The Linker^{A} of embodiment 225, wherein R¹⁶ is

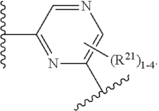

228. The Linker^{A} of embodiment 217, wherein R¹⁶ is pyridizinyl.
229. The Linker^{A} of embodiment 228, wherein R¹⁶ is

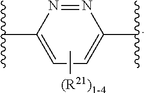

230. The Linker^{A} of embodiment 228, wherein R¹⁶ is

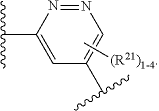

231. The Linker^{A} of any one of embodiments 1-193, wherein R¹⁶ is alkynyl.
232. The Linker^{A} of any one of embodiments 1-193, wherein R¹⁶ is alkoxy.
233. The Linker^{A} of any one of embodiments 1-232, wherein R¹⁷ is bond.
234. The Linker^{A} of any one of embodiments 1-232, wherein R¹⁷ is alkyl.
235. The Linker^{A} of any one of embodiments 1-232, wherein R¹⁷ is —C(O)—.
236. The Linker^{A} of any one of embodiments 1-232, wherein R¹⁷ is —C(O)O—.
237. The Linker^{A} of any one of embodiments 1-232, wherein R¹⁷ is —C(O)NR⁶—.
238. The Linker^{A} of any one of embodiments 1-232, wherein R¹⁷ is —NR⁶C(O)—.
239. The Linker^{A} of any one of embodiments 1-232, wherein R¹⁷ is —NR⁶C.
240. The Linker^{A} of any one of embodiments 1-232, wherein R¹⁷ is —O—.
241. The Linker^{A} of any one of embodiments 1-232, wherein R¹⁷ is —C(R²¹R²¹)—.
242. The Linker^{A} of embodiment 241, wherein both R²¹ groups are hydrogen.
243. The Linker^{A} of any one of embodiments 1-232, wherein R¹⁷ is —CH₂CH₂—[O—(CH₂)₂]ₙ—O—.
244. The Linker^{A} of any one of embodiments 1-232, wherein R¹⁷ is aryl.
245. The Linker^{A} of embodiment 244, wherein R¹⁷ is phenyl.
246. The Linker^{A} of embodiment 244, wherein R¹⁷ is naphthyl.
247. The Linker^{A} of any one of embodiments 1-232, wherein R¹⁷ is heterocycle.
248. The Linker^{A} of embodiment 247, wherein R¹⁷ is piperidinyl.
249. The Linker^{A} of embodiment 248, wherein R¹⁷ is

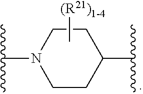

250. The Linker^{A} of embodiment 248, wherein R¹⁷ is

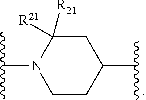

251. The Linker^{A} of embodiment 247, wherein R¹⁷ is piperizinyl.
252. The Linker^{A} of embodiment 251, wherein R¹⁷ is

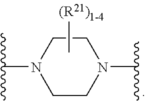

253. The Linker^{A} of embodiment 247, wherein R¹⁷ is pyrrolidinyl.
254. The Linker^{A} of embodiment 253, wherein R¹⁷ is

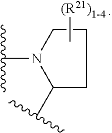

255. The Linker^{A} of embodiment 253, wherein R¹⁷ is

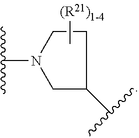

256. The Linker^{A} of any one of embodiments 1-232, wherein R¹⁷ is heteroaryl.
257. The Linker^{A} of embodiment 256, wherein R¹⁷ is pyridinyl.
258. The Linker^{A} of embodiment 257, wherein R¹⁷ is

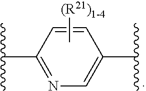

259. The Linker^{A} of embodiment 257, wherein R¹⁷ is

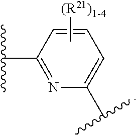

260. The Linker^{A} of embodiment 257, wherein R¹⁷ is

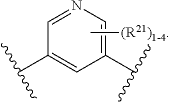

261. The Linker^{A} of embodiment 256, wherein R¹⁷ is pyrimidinyl.
262. The Linker^{A} of embodiment 261, wherein R¹⁷ is

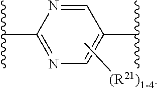

263. The Linker^{A} of embodiment 261, wherein R¹⁷ is

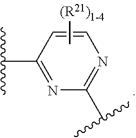

264. The Linker^{A} of embodiment 256, wherein R¹⁷ is pyrazinyl.
265. The Linker^{A} of embodiment 264, wherein R¹⁷ is

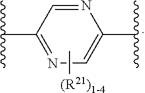

266. The Linker^{A} of embodiment 264, wherein R¹⁷ is

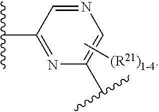

267. The Linker^{A} of embodiment 256, wherein R¹⁷ is pyridizinyl.
268. The Linker^{A} of embodiment 267, wherein R¹⁷ is

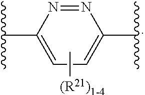

269. The Linker^{A} of embodiment 267, wherein R¹⁷ is

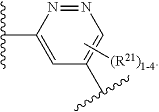

270. The Linker^{A} of any one of embodiments 1-232, wherein R¹⁷ is alkynyl.
271. The Linker^{A} of any one of embodiments 1-232, wherein R¹⁷ is alkoxy.
272. The Linker^{A} of any one of embodiments 1-271, wherein R¹⁸ is bond.
273. The Linker^{A} of any one of embodiments 1-271, wherein R¹⁸ is alkyl.
274. The Linker^{A} of any one of embodiments 1-271, wherein R¹⁸ is —C(O)—.
275. The Linker^{A} of any one of embodiments 1-271, wherein R¹⁸ is —C(O)O—.
276. The Linker^{A} of any one of embodiments 1-271, wherein R¹⁸ is —C(O)NR⁶—.
277. The Linker^{A} of any one of embodiments 1-271, wherein R¹⁸ is —NR⁶C(O)—.
278. The Linker^{A} of any one of embodiments 1-271, wherein R¹⁸ is —S(O)—.
279. The Linker^{A} of any one of embodiments 1-271, wherein R¹⁸ is —S(O)₂—.
280. The Linker^{A} of any one of embodiments 1-271, wherein R¹⁸ is —C(R²¹R²¹)—.
281. The Linker^{A} of embodiment 280, wherein both R²¹ groups are hydrogen.
282. The Linker^{A} of any one of embodiments 1-271, wherein R¹⁸ is —CH₂CH₂—[O—(CH₂)₂]ₙ—O—.
283. The Linker^{A} of any one of embodiments 1-271, wherein R¹⁸ is aryl.
284. The Linker^{A} of embodiment 283, wherein R¹⁸ is phenyl.
285. The Linker^{A} of embodiment 283, wherein R¹⁸ is naphthyl.
286. The Linker^{A} of any one of embodiments 1-271, wherein R⁸ is heterocycle.
287. The Linker^{A} of embodiment 286, wherein R¹⁸ is piperidinyl.
288. The Linker^{A} of embodiment 287, wherein R¹⁸ is

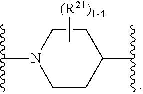

289. The Linker^{A} of embodiment 287, wherein R¹⁸ is

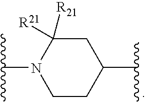

290. The Linker^{A} of embodiment 286, wherein R¹⁸ is piperizinyl.
291. The Linker^{A} of embodiment 290, wherein R¹⁸ is

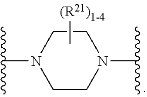

292. The Linker^{A} of embodiment 286, wherein R¹⁸ is pyrrolidinyl.
293. The Linker^{A} of embodiment 292, wherein R¹⁸ is

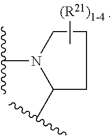

294. The Linker^{A} of embodiment 292, wherein R¹⁸ is

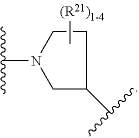

295. The Linker^{A} of any one of embodiments 1-271, wherein R¹⁸ is heteroaryl.
296. The Linker^{A} of embodiment 295, wherein R¹⁸ is pyridinyl.
297. The Linker^{A} of embodiment 296, wherein R¹⁸ is

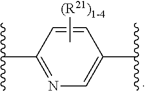

298. The Linker^{A} of embodiment 296, wherein R¹⁸ is

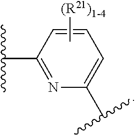

299. The Linker^{A} of embodiment 296, wherein R¹⁸ is

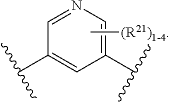

300. The Linker^{A} of embodiment 295, wherein R¹⁸ is pyrimidinyl.
301. The Linker^{A} of embodiment 300, wherein R¹⁸ is

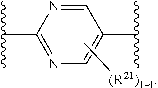

302. The Linker^{A} of embodiment 300, wherein R¹⁸ is

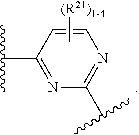

303. The Linker^{A} of embodiment 295, wherein R¹⁸ is pyrazinyl.
304. The Linker^{A} of embodiment 303, wherein R¹⁸ is

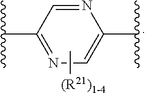

305. The Linker^{A} of embodiment 303, wherein R¹⁸ is

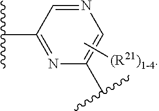

306. The Linker^{A} of embodiment 295, wherein R¹⁸ is pyridizinyl.
307. The Linker^{A} of embodiment 306, wherein R¹⁸ is

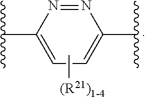

308. The Linker^{A} of embodiment 306, wherein R¹⁸ is

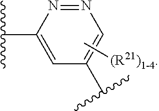

309. The Linker^{A} of any one of embodiments 1-271, wherein R¹⁸ is alkynyl.
310. The Linker^{A} of any one of embodiments 1-271, wherein R¹⁸ is alkoxy.
311. The Linker^{A} of any one of embodiments 1-310, wherein R¹⁹ is bond.
312. The Linker^{A} of any one of embodiments 1-310, wherein R¹⁹ is alkyl.
313. The Linker^{A} of any one of embodiments 1-310, wherein R¹⁹ is —C(O)—.
314. The Linker^{A} of any one of embodiments 1-310, wherein R¹⁹ is —C(O)O—.
315. The Linker^{A} of any one of embodiments 1-310, wherein R¹⁹ is —C(O)NR⁶—.
316. The Linker^{A} of any one of embodiments 1-310, wherein R¹⁹ is —NR⁶C(O)—.
317. The Linker^{A} of any one of embodiments 1-310, wherein R¹⁹ is —NR⁶—.
318. The Linker^{A} of any one of embodiments 1-310, wherein R¹⁹ is —O—.
319. The Linker^{A} of any one of embodiments 1-310, wherein R¹⁹ is —C(R²¹R²¹)—.
320. The Linker^{A} of embodiment 319, wherein both R²¹ groups are hydrogen.
321. The Linker^{A} of any one of embodiments 1-310, wherein R¹⁹ is —CH₂CH₂—[O—(CH₂)₂]ₙ—O—.
322. The Linker^{A} of any one of embodiments 1-310, wherein R¹⁹ is aryl.
323. The Linker^{A} of embodiment 322, wherein R¹⁹ is phenyl.
324. The Linker^{A} of embodiment 322, wherein R¹⁹ is naphthyl.
325. The Linker^{A} of any one of embodiments 1-310, wherein R¹⁹ is heterocycle.
326. The Linker^{A} of embodiment 325, wherein R¹⁹ is piperidinyl.
327. The Linker^{A} of embodiment 326, wherein R¹⁹ is

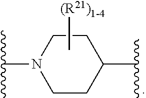

328. The Linker^{A} of embodiment 326, wherein R¹⁹ is

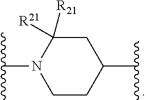

329. The Linker^{A} of embodiment 325, wherein R¹⁹ is piperizinyl.
330. The Linker^{A} of embodiment 329, wherein R¹⁹ is

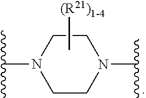

331. The Linker^{A} of embodiment 325, wherein R¹⁹ is pyrrolidinyl.
332. The Linker^{A} of embodiment 331, wherein R¹⁹ is

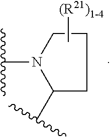

333. The Linker^{A} of embodiment 331, wherein R¹⁹ is

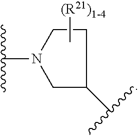

334. The Linker^{A} of any one of embodiments 1-310, wherein R¹⁹ is heteroaryl.
335. The Linker^{A} of embodiment 334, wherein R¹⁹ is pyridinyl.
336. The Linker^{A} of embodiment 335, wherein R¹⁹ is

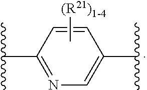

337. The Linker^{A} of embodiment 335, wherein R¹⁹ is

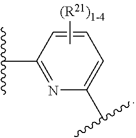

338. The Linker^{A} of embodiment 335, wherein R¹⁹ is

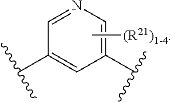

339. The Linker^{A} of embodiment 334, wherein R¹⁹ is pyrimidinyl.
340. The Linker^{A} of embodiment 339, wherein R¹⁹ is

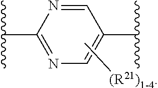

341. The Linker^{A} of embodiment 339, wherein R¹⁹ is

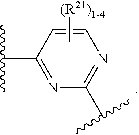

342. The Linker^{A} of embodiment 334, wherein R¹⁹ is pyrazinyl.
343. The Linker^{A} of embodiment 342, wherein R¹⁹ is

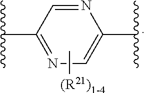

344. The Linker^{A} of embodiment 342, wherein R¹⁹ is

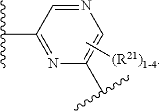

345. The Linker^{A} of embodiment 334, wherein R¹⁹ is pyridizinyl.
346. The Linker^{A} of embodiment 345, wherein R¹⁹ is

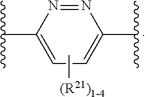

347. The Linker^{A} of embodiment 345, wherein R¹⁹ is

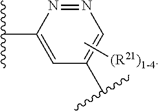

348. The Linker^{A} of any one of embodiments 1-310, wherein R¹⁹ is alkynyl.
349. The Linker^{A} of any one of embodiments 1-310, wherein R¹⁹ is alkoxy.
350. The Linker^{A} of any one of embodiments 1-350, wherein R²⁰ is bond.
351. The Linker^{A} of any one of embodiments 1-350, wherein R²⁰ is alkyl.
352. The Linker^{A} of any one of embodiments 1-350, wherein R²⁰ is —C(O)—.
353. The Linker^{A} of any one of embodiments 1-350, wherein R²⁰ is —C(O)O—.
354. The Linker^{A} of any one of embodiments 1-350, wherein R²⁰ is —C(O)NR⁶—.
355. The Linker^{A} of any one of embodiments 1-350, wherein R²⁰ is —NR⁶C(O)—.
356. The Linker^{A} of any one of embodiments 1-350, wherein R²⁰ is —S(O)—.
357. The Linker^{A} of any one of embodiments 1-350, wherein R²⁰ is —S(O)₂—.
358. The Linker^{A} of any one of embodiments 1-350, wherein R²⁰ is —C(R²¹R²¹)—.
359. The Linker^{A} of embodiment 359, wherein both R²¹ groups are hydrogen.
360. The Linker^{A} of any one of embodiments 1-350, wherein R²⁰ is —CH₂CH₂—[O—(CH₂)₂]ₙ—O—.
361. The Linker^{A} of any one of embodiments 1-350, wherein R²⁰ is aryl.
362. The Linker^{A} of embodiment 362, wherein R²⁰ is phenyl.
363. The Linker^{A} of embodiment 362, wherein R²⁰ is naphthyl.
364. The Linker^{A} of any one of embodiments 1-350, wherein R²⁰ is heterocycle.
365. The Linker^{A} of embodiment 365, wherein R²⁰ is piperidinyl.
366. The Linker^{A} of embodiment 366, wherein R²⁰ is

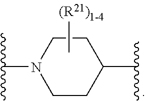

367. The Linker^{A} of embodiment 366, wherein R²⁰ is

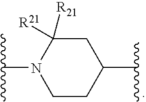

368. The Linker^{A} of embodiment 365, wherein R²⁰ is piperizinyl.
369. The Linker^{A} of embodiment 369, wherein R²⁰ is

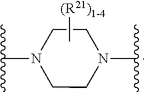

370. The Linker^{A} of embodiment 365, wherein R²⁰ is pyrrolidinyl.
371. The Linker^{A} of embodiment 371, wherein R²⁰ is

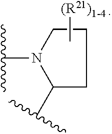

372. The Linker^{A} of embodiment 371, wherein R²⁰ is

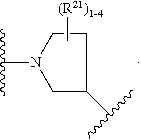

373. The Linker^{A} of any one of embodiments 1-350, wherein R²⁰ is heteroaryl.
374. The Linker^{A} of embodiment 374, wherein R²⁰ is pyridinyl.
375. The Linker^{A} of embodiment 375, wherein R²⁰ is

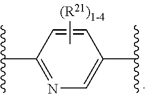

376. The Linker^{A} of embodiment 375, wherein R²⁰ is

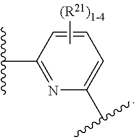

377. The Linker^{A} of embodiment 375, wherein R²⁰ is

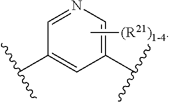

378. The Linker^{A} of embodiment 374, wherein R²⁰ is pyrimidinyl.
379. The Linker^{A} of embodiment 379, wherein R²⁰ is

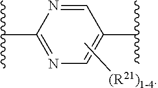

380. The Linker^{A} of embodiment 379, wherein R²⁰ is

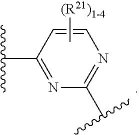

381. The Linker^{A} of embodiment 374, wherein R²⁰ is pyrazinyl.
382. The Linker^{A} of embodiment 382, wherein R²⁰ is

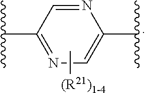

383. The Linker^{A} of embodiment 382, wherein R²⁰ is

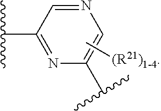

384. The Linker^{A} of embodiment 374, wherein R²⁰ is pyridizinyl.
385. The Linker^{A} of embodiment 385, wherein R²⁰ is

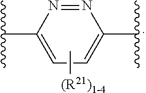

386. The Linker^{A} of embodiment 385, wherein R²⁰ is

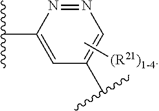

387. The Linker of any one of embodiments 1-350, wherein R²⁰ is alkynyl.
388. The Linker^{A} of any one of embodiments 1-350, wherein R²⁰ is alkoxy.

In certain embodiments, a divalent residue of an amino acid is selected from

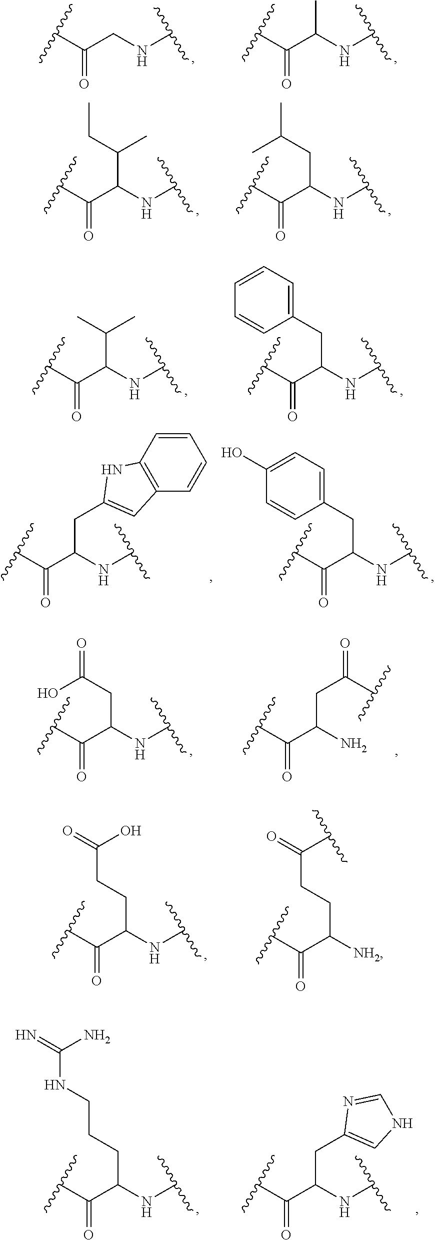

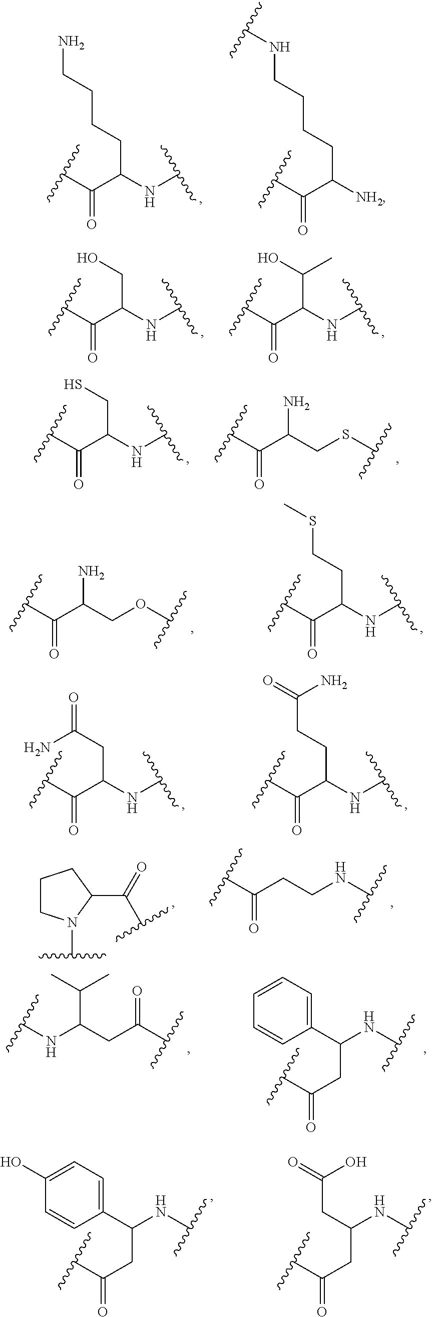

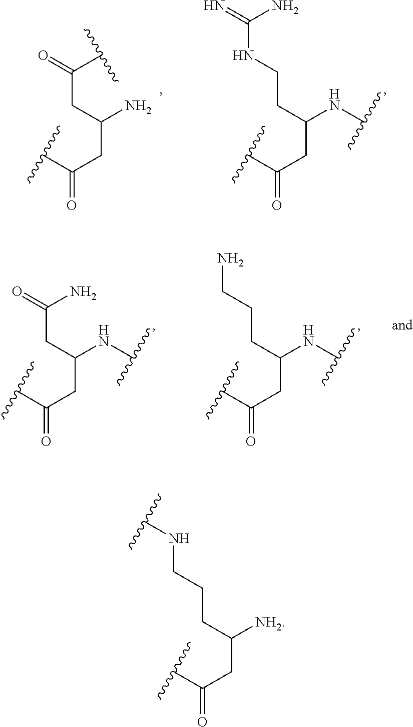 [SMILES: *C(=O)CC(N)CC(*)=O ; *NC(CCNC(=N)N)CC(*)=O ; *NC(CC(*)=O)CC(N)=O ; *NC(CCCN)CC(*)=O ; *NCCCC(N)CC(*)=O]

wherein the amino acid can be oriented in either direction and wherein the amino acid can be in the L- or D-form.

In certain embodiments, a divalent residue of a dicarboxylic acid is generated from a nucleophilic addition reaction:

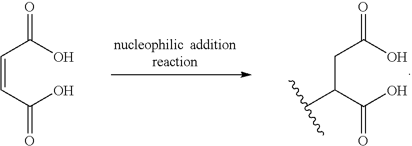

Non-limiting embodiments of a divalent residue of a dicarboxylic acid generated from a nucleophilic addition reaction include:

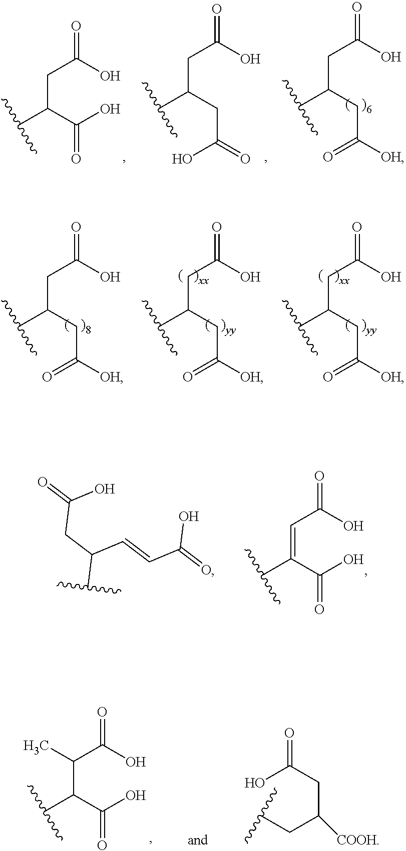

As used in the embodiments herein, xx is independently selected from 0, 1, 2, 3, 4, 5, 6, 7, 8, 9, 10, 11, 12, 13, 14, 15, 16, 17, 18, 19, 20, 21, 22, 23, 24, and 25.

As used in the embodiments herein, yy is independently selected from 0, 1, 2, 3, 4, 5, 6, 7, 8, 9, 10, 11, 12, 13, 14, 15, 16, 17, 18, 19, 20, 21, 22, 23, 24, and 25.

In certain embodiments, a divalent residue of a dicarboxylic acid is generated from a condensation reaction:

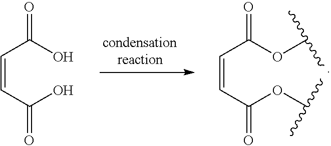

Non-limiting embodiments of a divalent residue of a dicarboxylic acid generated from a condensation include:

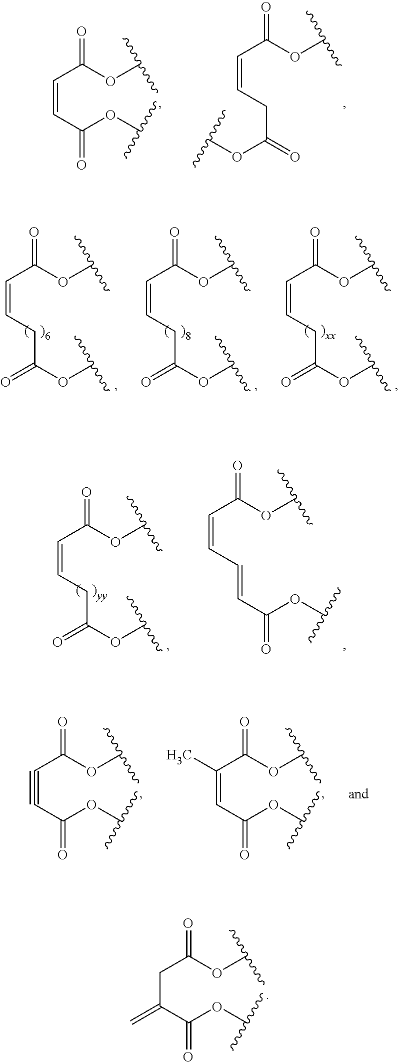

Non-limiting embodiments of a divalent residue of a saturated dicarboxylic acid include:

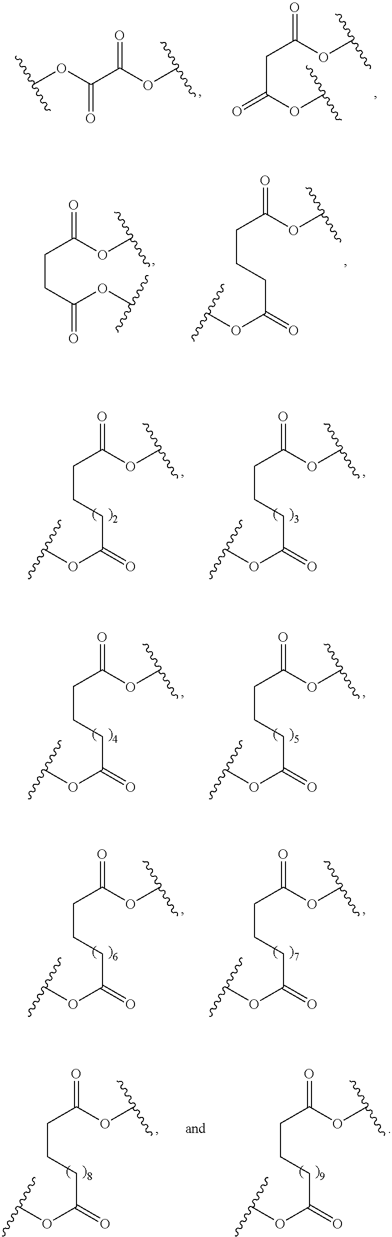

Non-limiting embodiments of a divalent residue of a saturated dicarboxylic acid include:

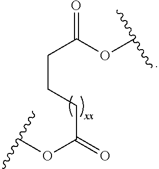

Non-limiting embodiments of a divalent residue of a saturated monocarboxylic acid is selected from butyric acid (—OC(O)(CH₂)₂CH₂—), caproic acid (—OC(O)(CH₂)₄CH₂—), caprylic acid (—OC(O)(CH₂)₅CH₂—), capric acid (—OC(O)(CH₂)₈CH₂—), lauric acid (—OC(O)(CH₂)₁₀CH₂—), myristic acid (—OC(O)(CH₂)₁₂CH₂—), pentadecanoic acid (—OC(O)(CH₂)₁₃CH₂—), palmitic acid (—OC(O)(CH₂)₁₄CH₂—), stearic acid (—OC(O)(CH₂)₁₆CH₂—), behenic acid (—OC(O)(CH₂)₂₀CH₂—), and lignoceric acid (—OC(O)(CH₂)₂₂CH₂—);

Non-limiting embodiments of a divalent residue of a fatty acid include residues selected from linoleic acid, palmitoleic acid, vaccenic acid, paullinic acid, oleic acid, elaidic acid, gondoic acid, gadoleic acid, nervonic acid, myristoleic acid, and erucic acid:

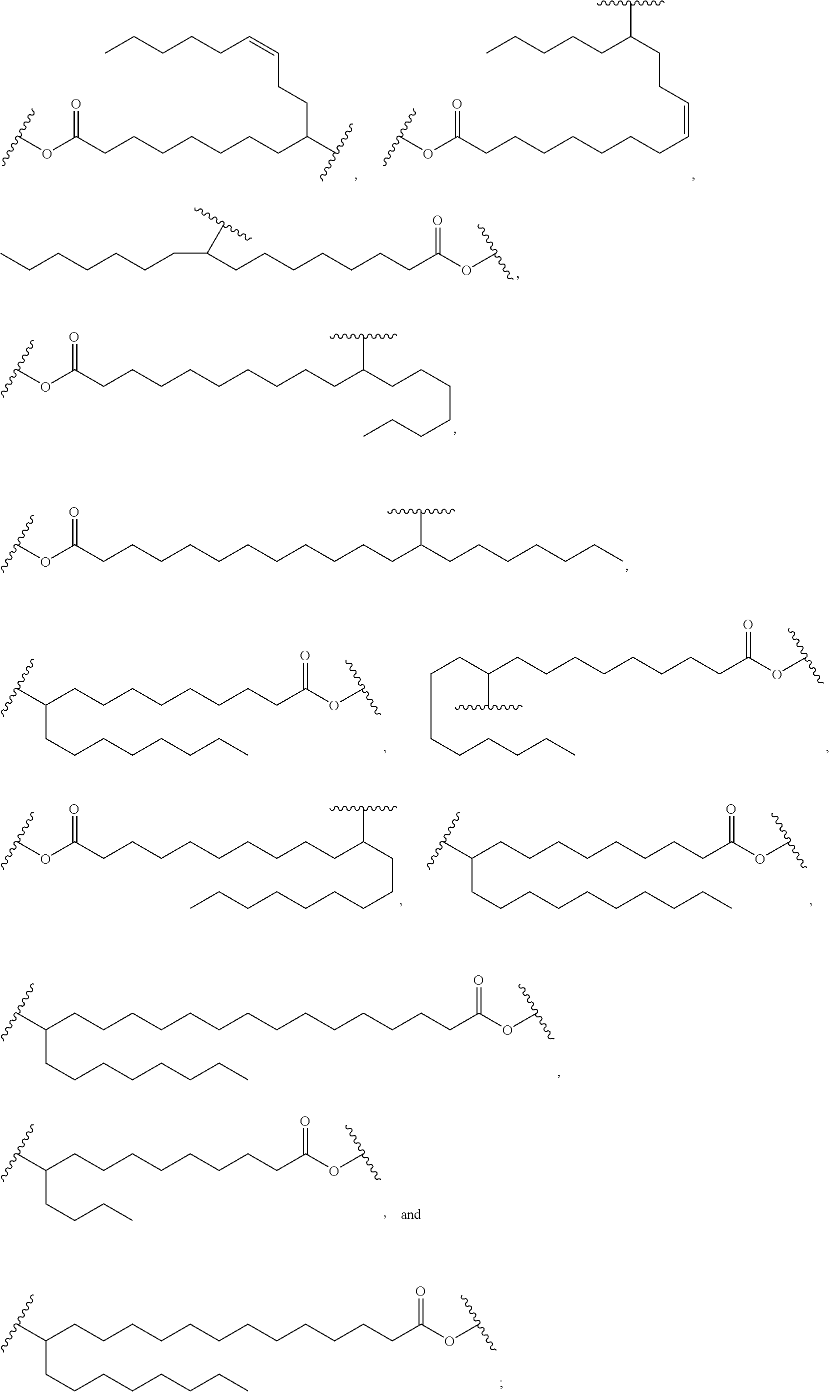

Non-limiting embodiments of a divalent residue of a fatty acid is selected from linoleic acid (—C(O)(CH₂)₇(CH)₂CH₂(CH)₂(CH₂)₄CH₂—), docosahexaenoic acid (—C(O)(CH₂)₂(CHCHCH₂)₆CH₂—), eicosapentaenoic acid (—C(O)(CH₂)₃(CHCHCH₂)₅CH₂—), alpha-linolenic acid (—C(O)(CH₂)₇(CHCHCH₂)₃CH₂—) stearidonic acid (—C(O)(CH₂)₄(CHCHCH₂)₄CH₂—), y-linolenic acid (—C(O)(CH₂)₄(CHCHCH₂)₃(CH₂)₃CH₂—), arachidonic acid (—C(O)(CH₂)₃, (CHCHCH₂)₄(CH₂)₄CH₂—), docosatetraenoic acid (—C(O)(CH₂)₅(CHCHCH₂)₄(CH₂)₄CH₂—), palmitoleic acid (—C(O)(CH₂)₇CHCH(CH₂)₅CH₂—), vaccenic acid (—C(O)(CH₂)₉CHCH(CH₂)₅CH₂—), paullinic acid (—C(O)(CH₂)₁₁CHCH(CH₂)₅CH₂—), oleic acid (—C(O)(CH₂)₇CHCH(CH₂)₇CH₂—), elaidic acid (—C(O)(CH₂)₇CHCH(CH₂)₇CH₂—), gondoic acid (—C(O)(CH₂)₉CHCH(CH₂)₇CH₂—), gadoleic acid (—C(O)(CH₂)₇CHCH(CH₂)₉CH₂—), nervonic acid (—C(O)(CH₂)₁₃CHCH(CH₂)₇CH₂—), mead acid (—C(O)(CH₂)₃(CHCHCH₂)₃(CH₂)₆CH₂—), myristoleic acid (—C(O)(CH₂)₇CHCH(CH₂)₃CH₂—), and erucic acid (—C(O)(CH₂)₁₁CHCH(CH₂)₇CH₂—).

In certain embodiments Linker^{C} is selected from:

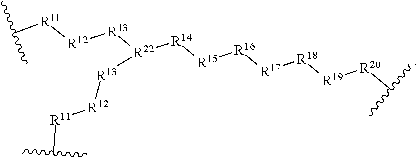

wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹;
and the remaining variables are as defined herein.

In certain embodiments Linker^{D} is selected from:

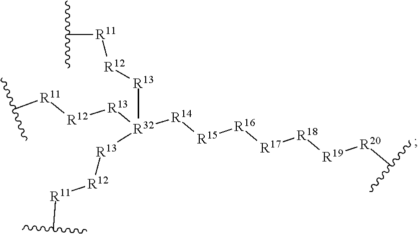

wherein:
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹;
X⁻ is an anionic group, for example Br⁻ or Cl⁻; and
all other variables are as defined herein.

In certain embodiments Linker^{A} is selected from:

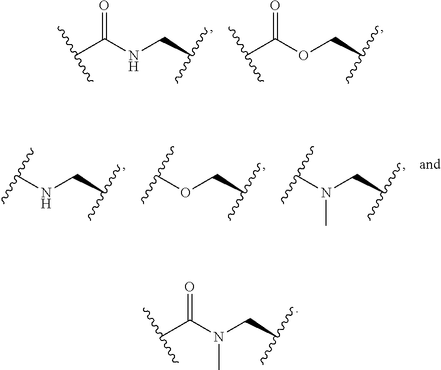

In certain embodiments Linker^{A} is selected from:

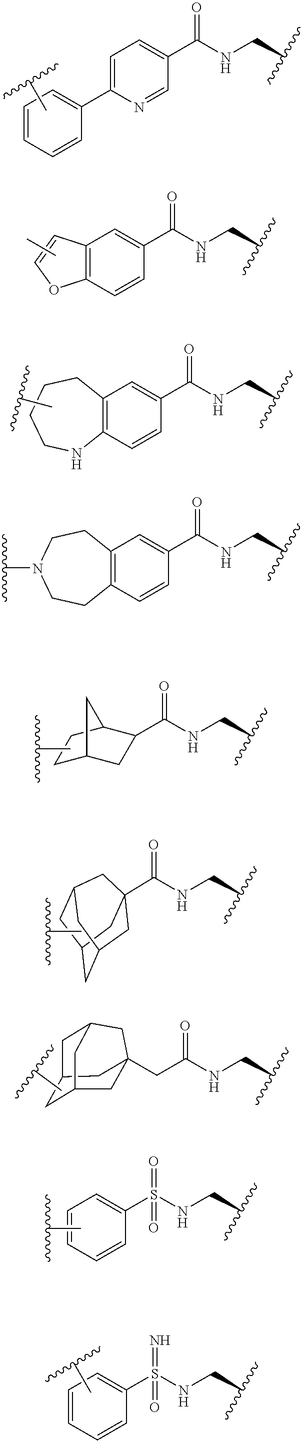

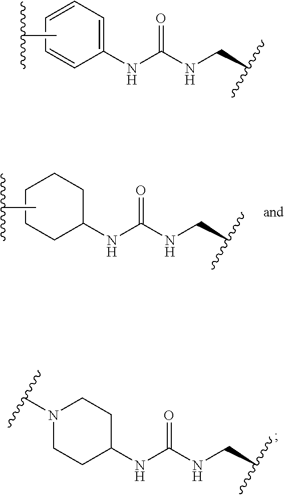

each of which is optionally substituted with 1, 2, 3, or 4 optional substituents as defined herein.

In certain embodiments Linker^{A} is selected from:

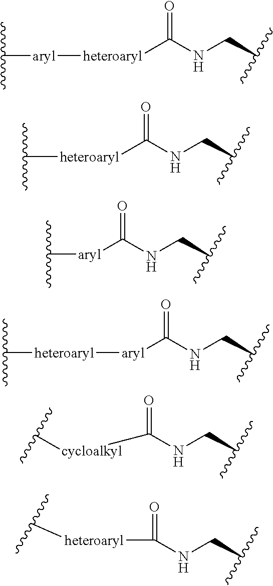

each of which is optionally substituted with 1, 2, 3, or 4 optional substituents as defined herein.

In certain embodiments Linker^{A} is selected from:

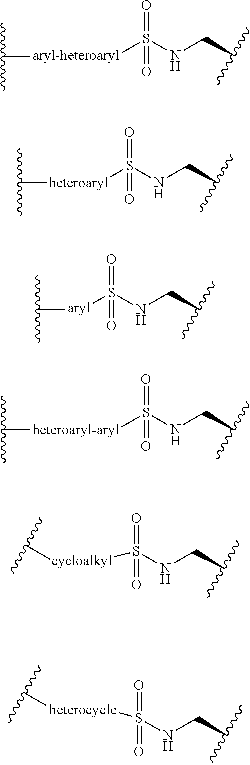

each of which is optionally substituted with 1, 2, 3, or 4 optional substituents as defined herein.

In certain embodiments Linker^{A} is selected from:

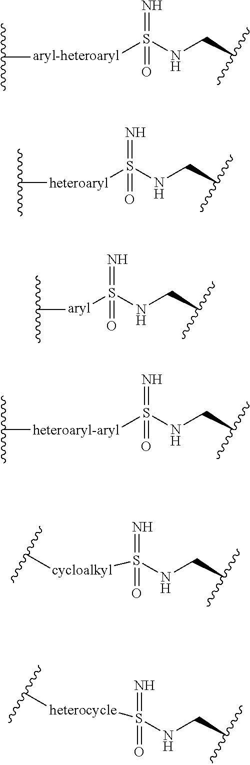

each of which is optionally substituted with 1, 2, 3, or 4 optional substituents as defined herein.

In certain embodiments Linker^{A} is selected from:

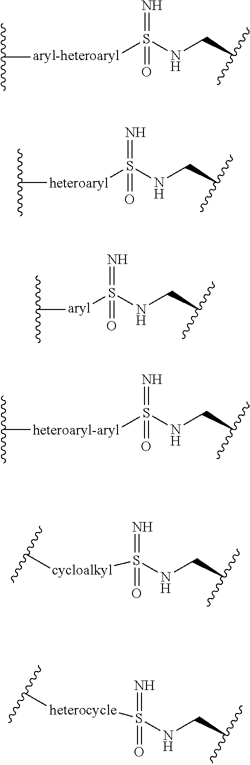

each of which is optionally substituted with 1, 2, 3, or 4 optional substituents as defined herein.

In certain embodiments Linker^{A} is selected from:

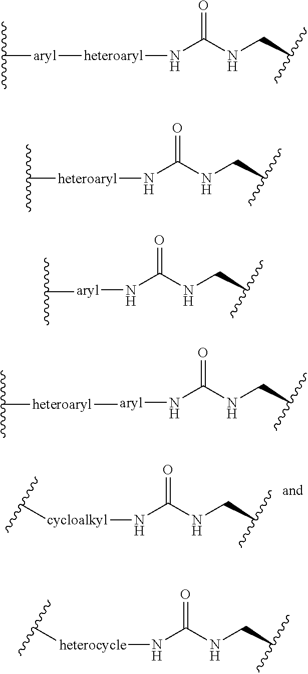

each of which is optionally substituted with 1, 2, 3, or 4 optional substituents as defined herein.

In certain embodiments Linker^{A} is selected from:

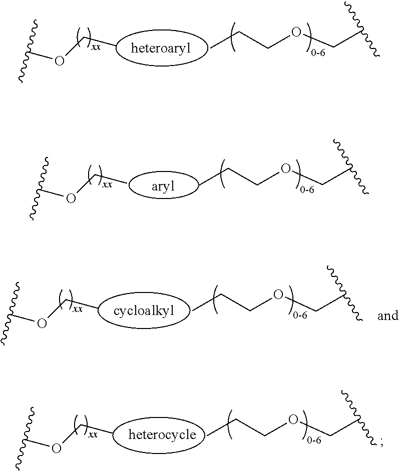

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

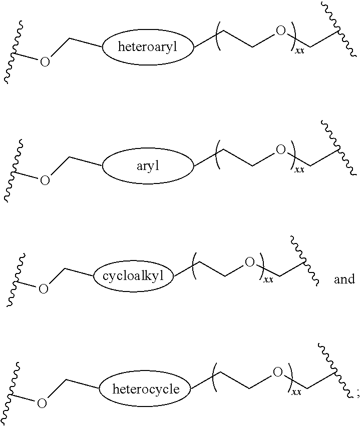

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selelcted from:

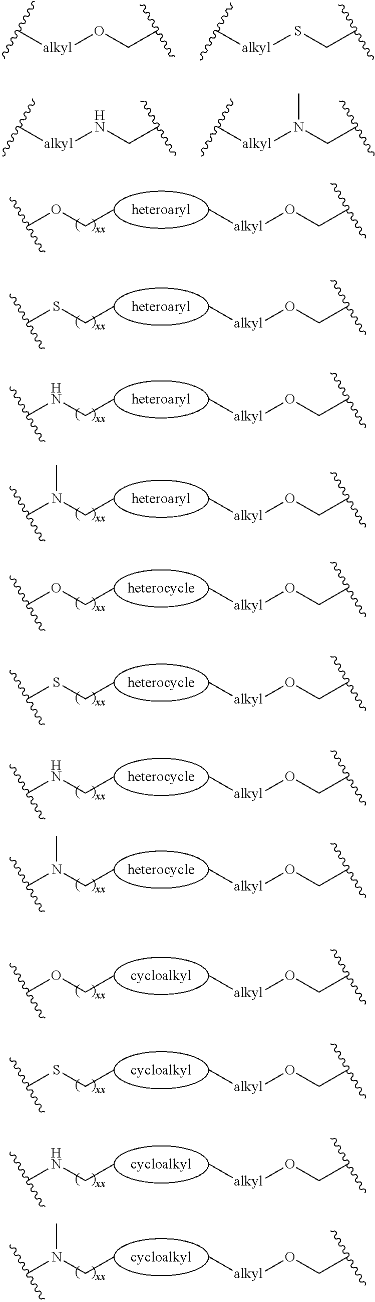

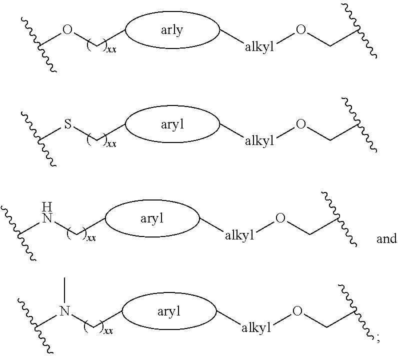

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

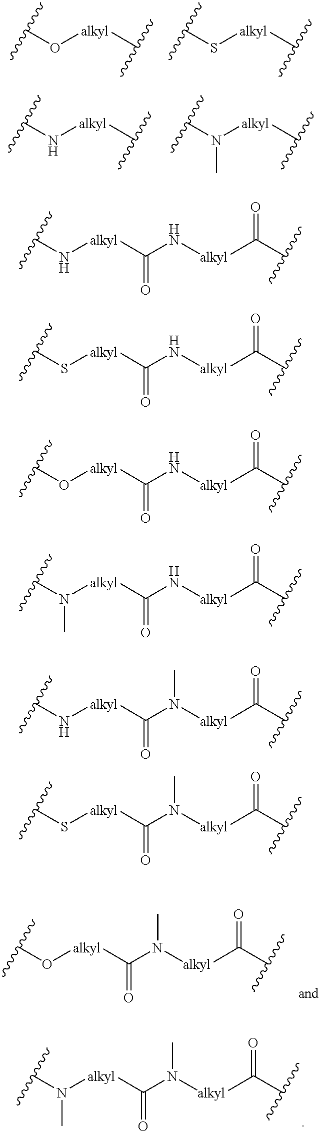

In certain embodiments Linker^{B} is selected from:

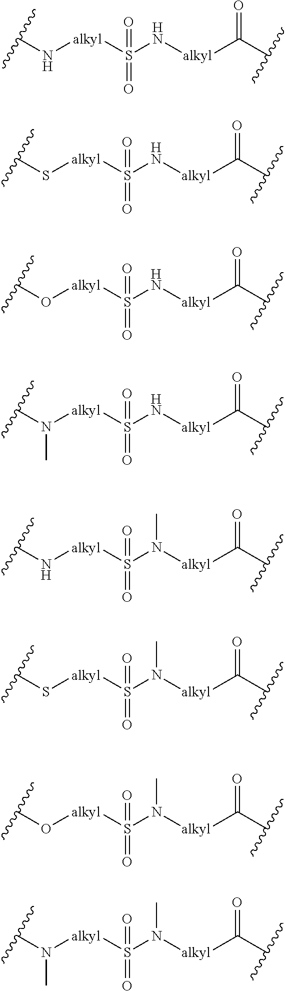

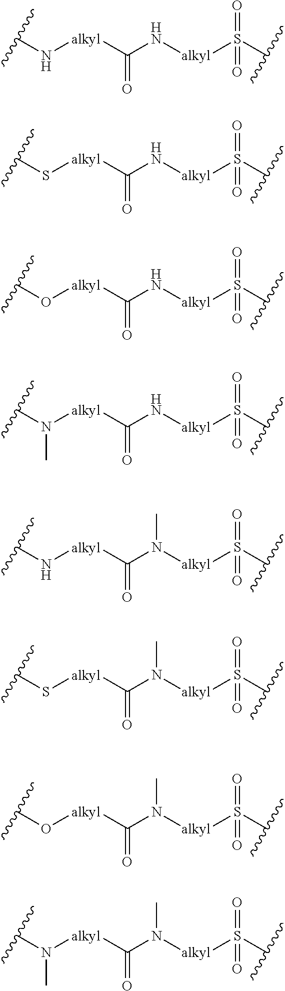

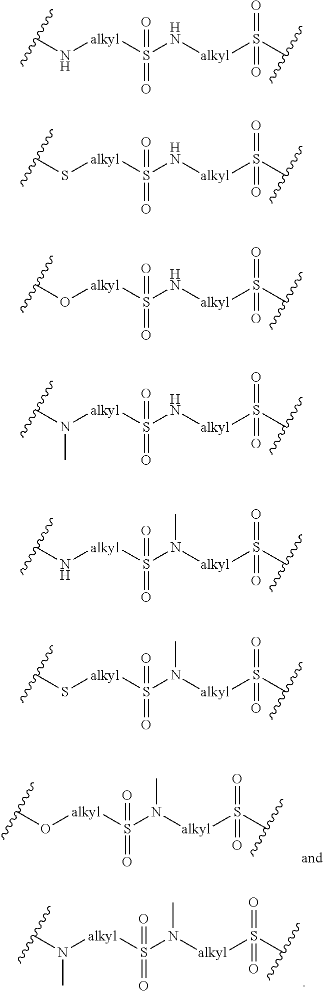

In certain embodiments Linker^{B}, Linker^{C}, or Linker^{D} is selected from:

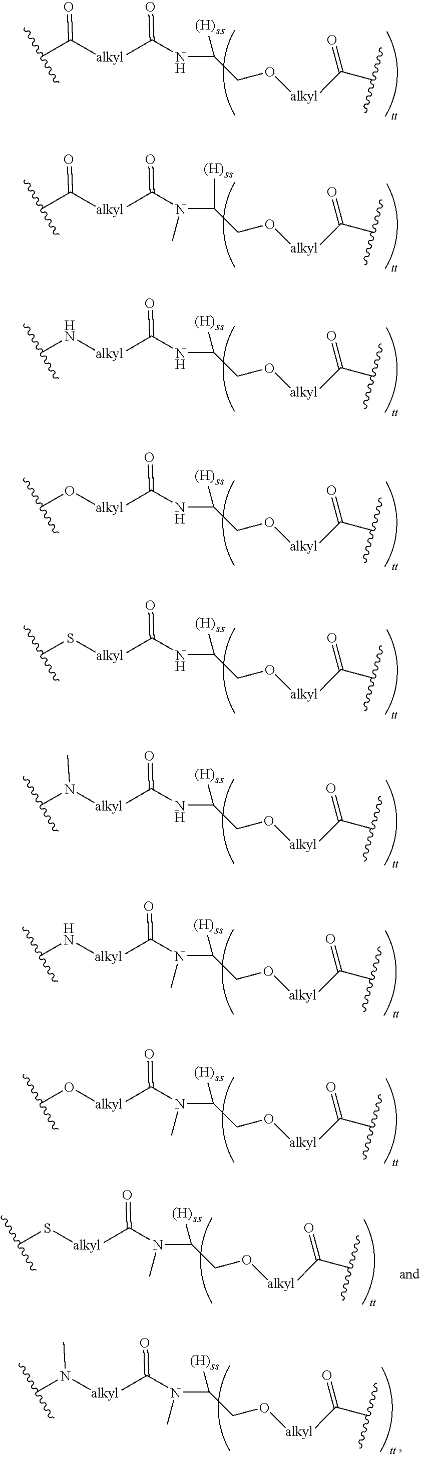

wherein tt is independently selected from 1, 2, or 3 and ss is 3 minus tt.

In certain embodiments Linker^{B}, Linker^{C}, or Linker^{D} is selected from:

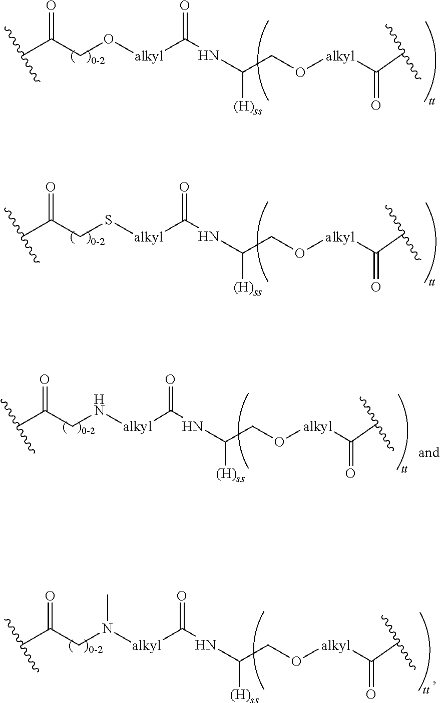

wherein tt and ss are as defined herein.

In certain embodiments Linker^{B}, Linker^{C}, or Linker^{D} is selected from:

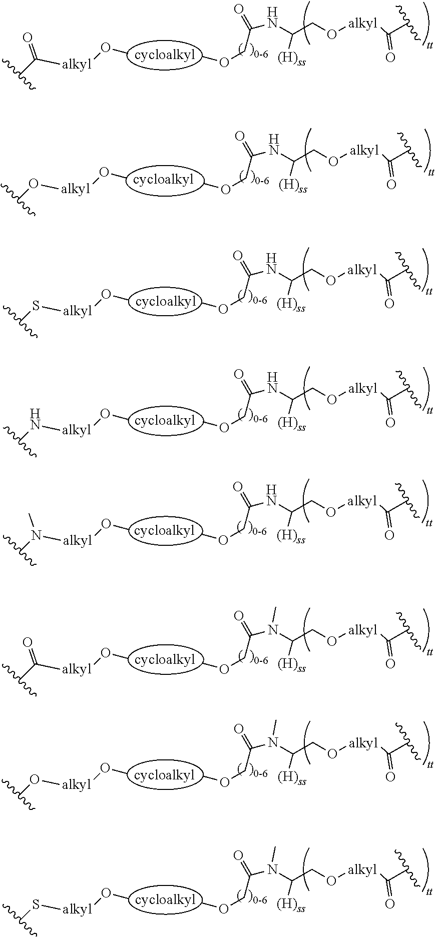

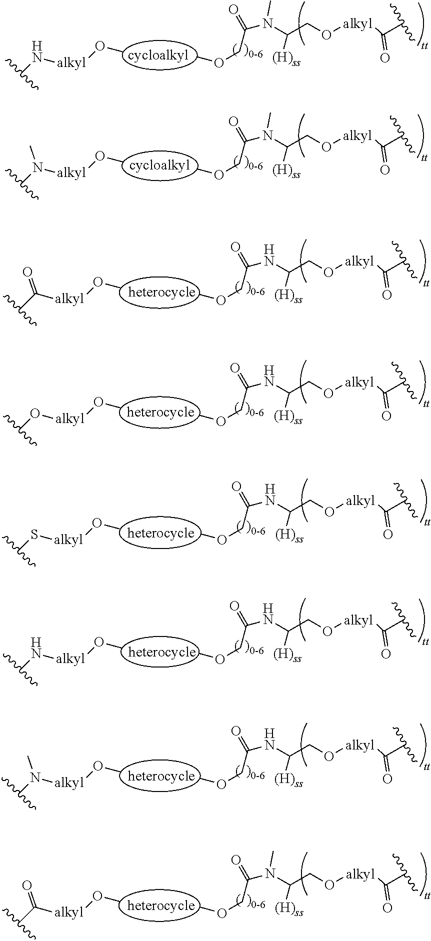

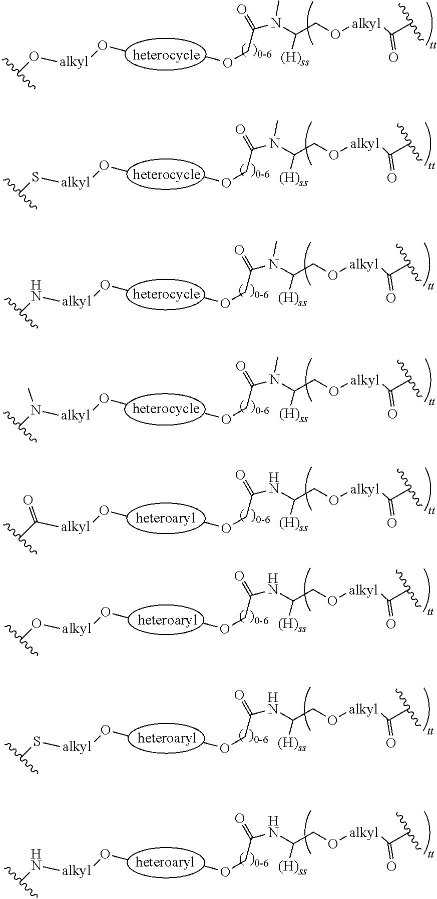

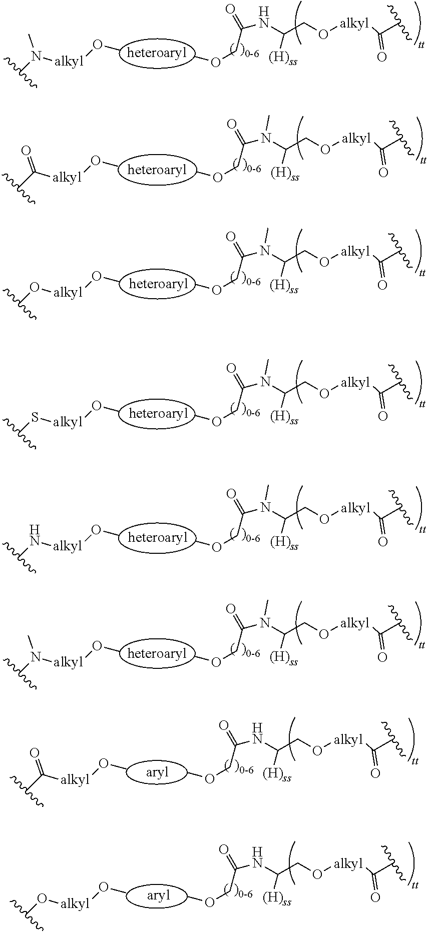

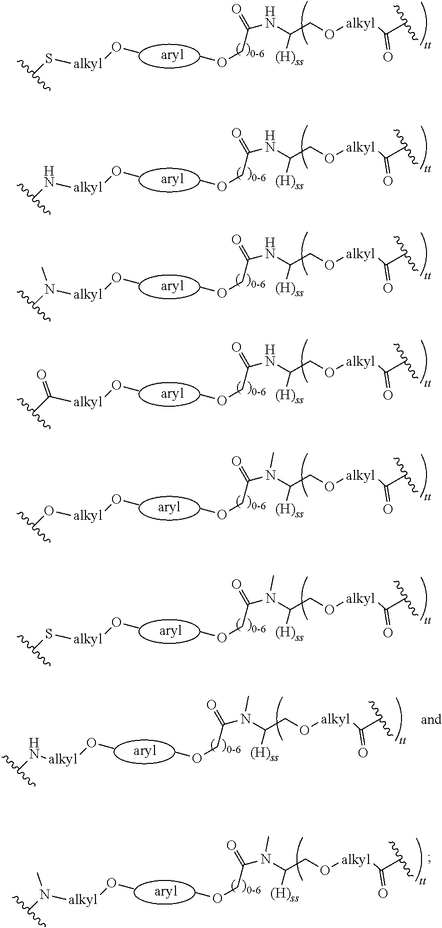

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

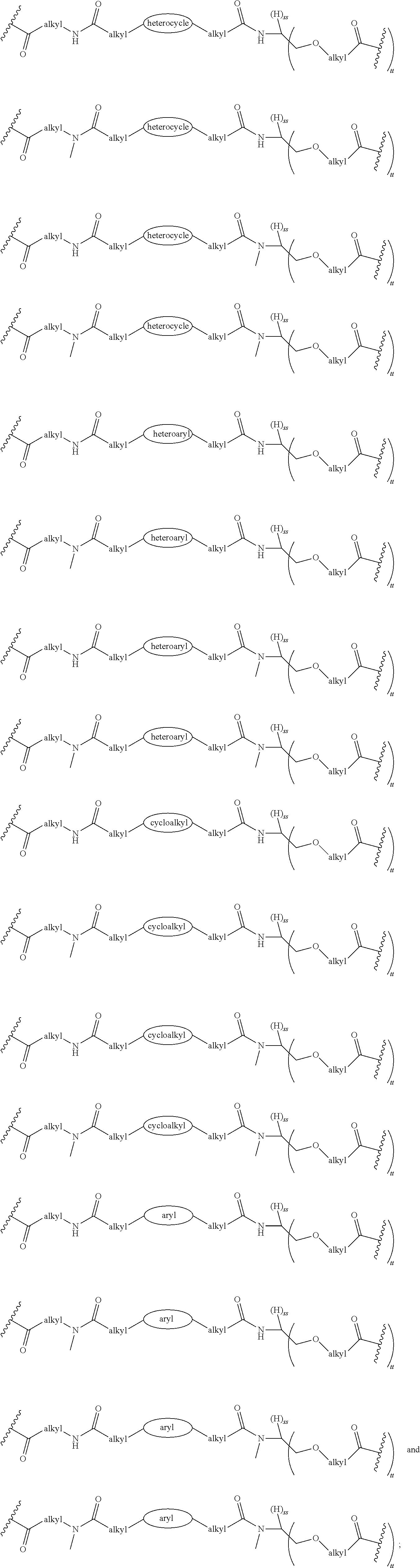

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{B}, Linker^{C}, or Linker^{D} is selected from:

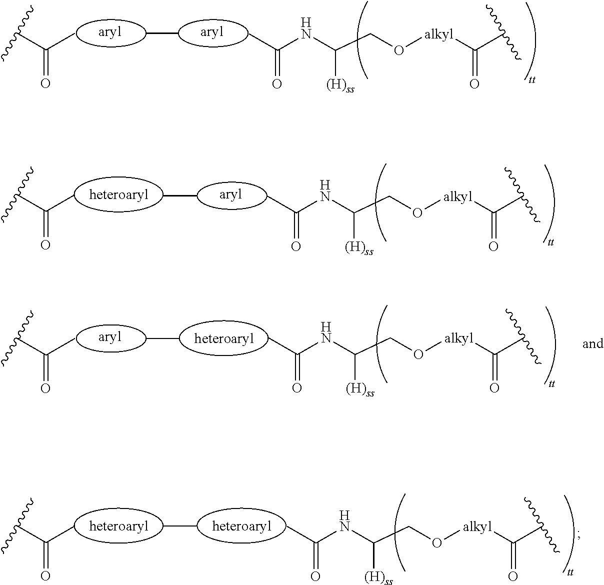

wherein each heteroaryl and aryl can optionally be substituted with 1, 2, 3, or 4 of any combination of halogen, alkyl, haloalkyl, aryl, heteroaryl, heterocycle, or cycloalkyl, as allowed by valence; and tt and ss are as defined herein.

In certain embodiments Linker^{A} is selected from:

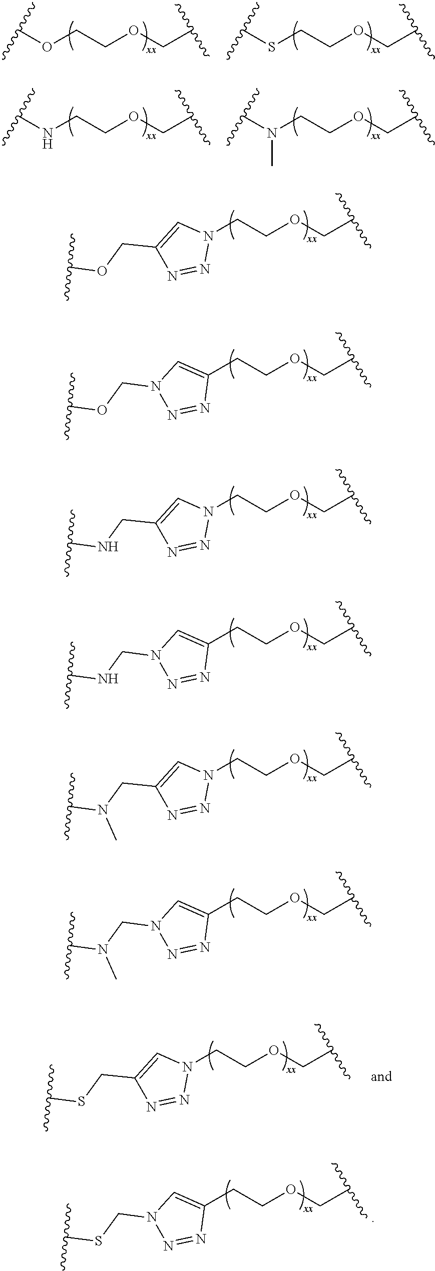

In certain embodiments Linker^{A} is selected from:

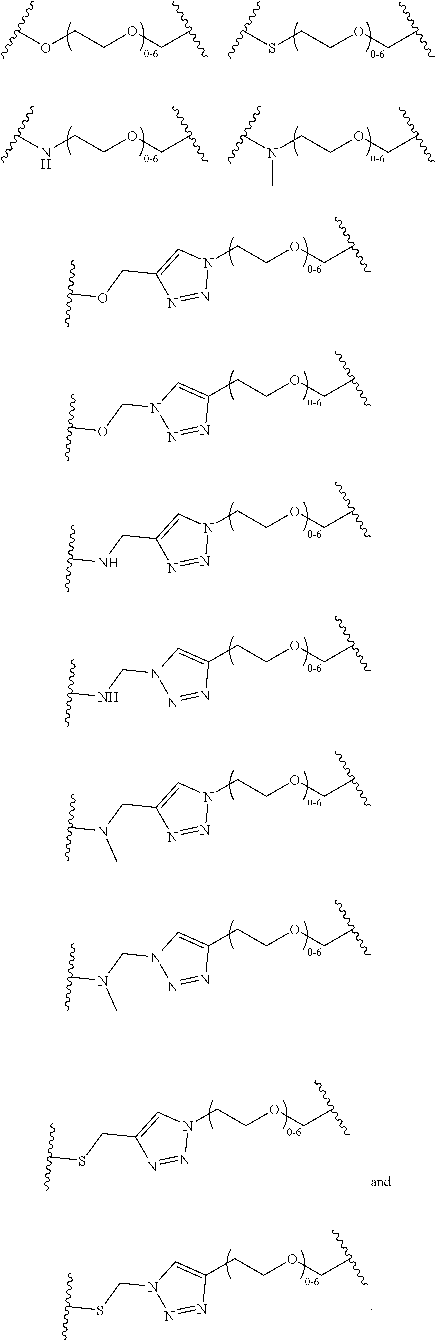

In certain embodiments Linker^{A} is selected from:

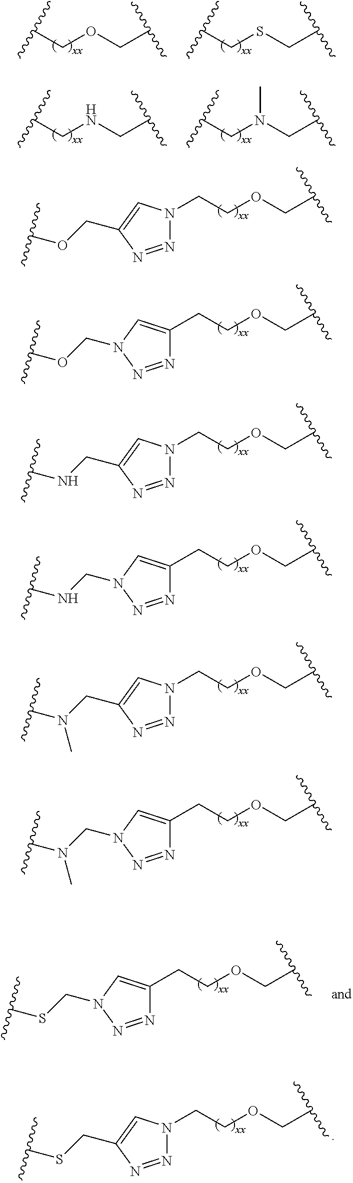

In certain embodiments Linker^{A} is selected from:

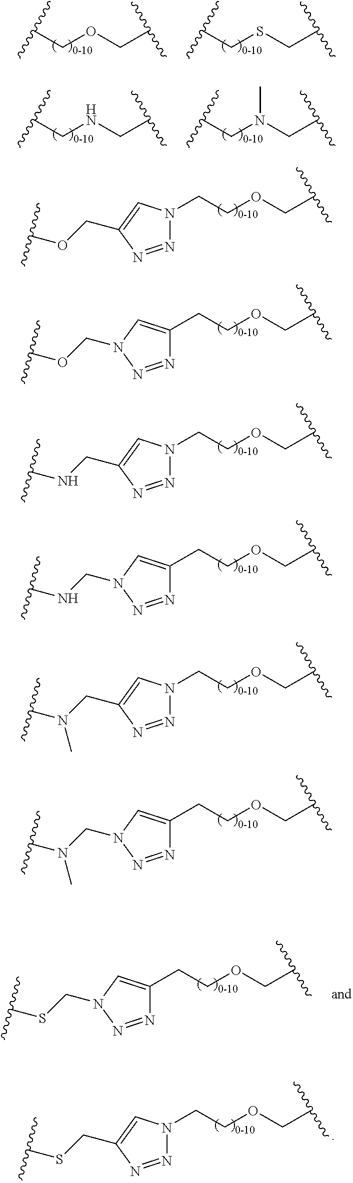

In certain embodiments Linker^{B} is selected from:

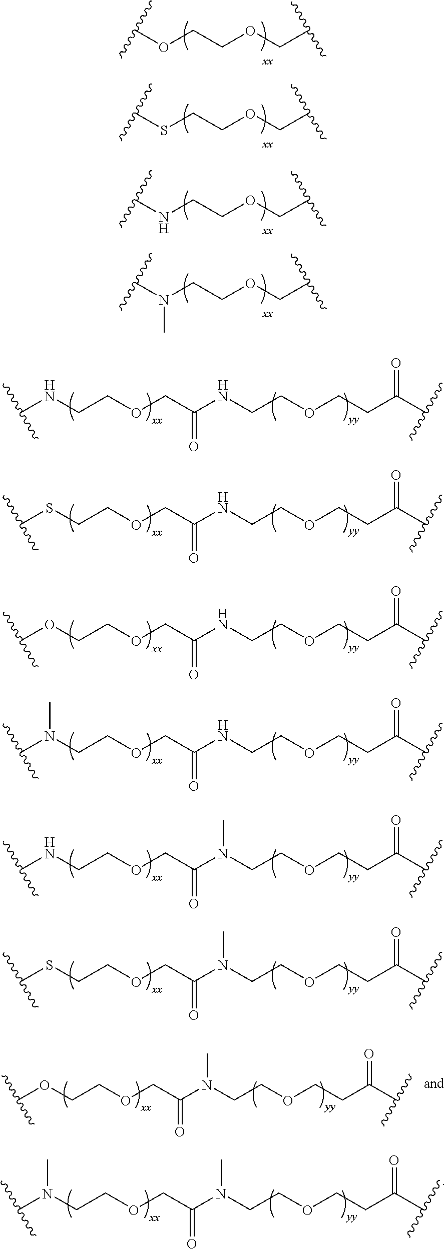

In certain embodiments Linker^{B} is selected from:

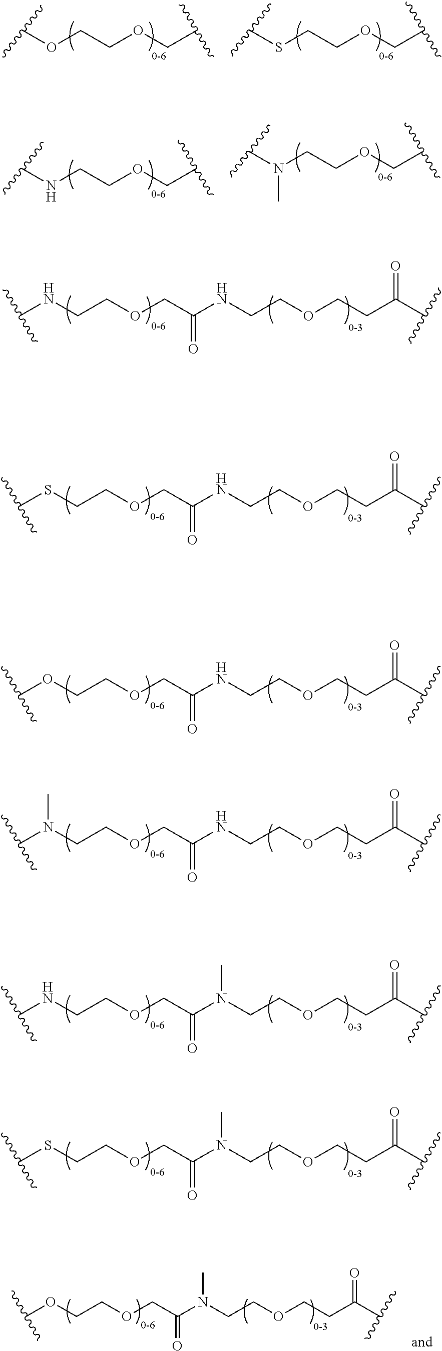

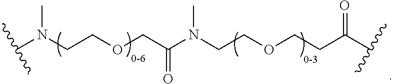

In certain embodiments Linker^{B} is selected from:

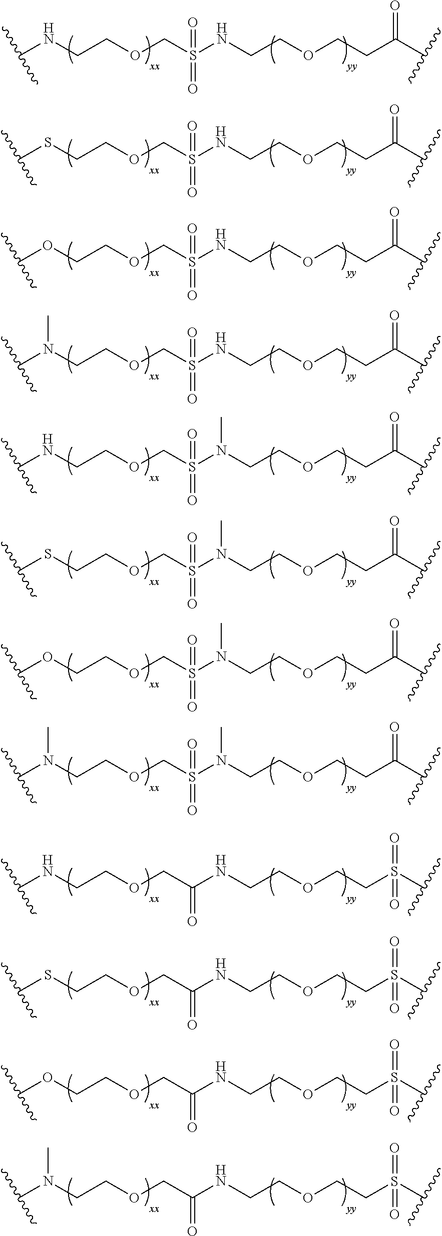

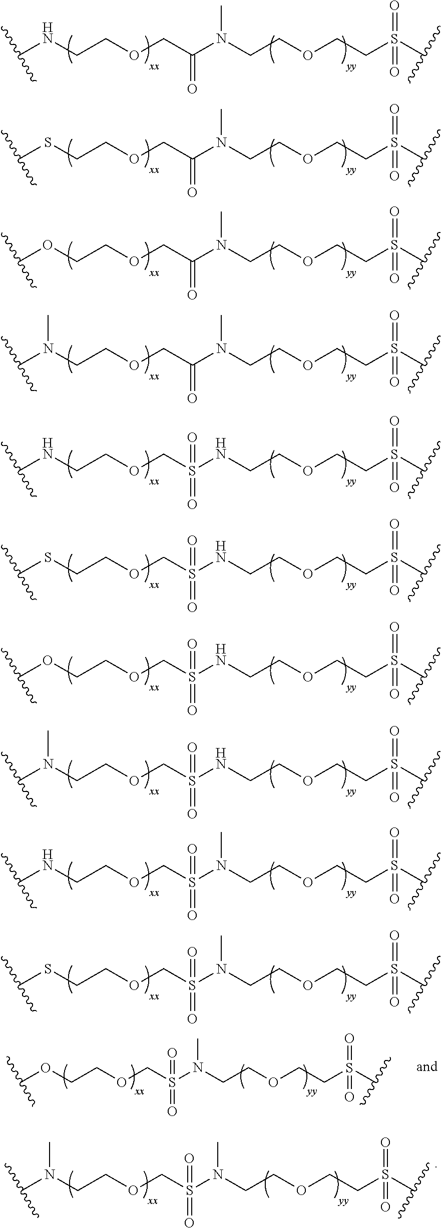

In certain embodiments Linker^{B} is selected from:

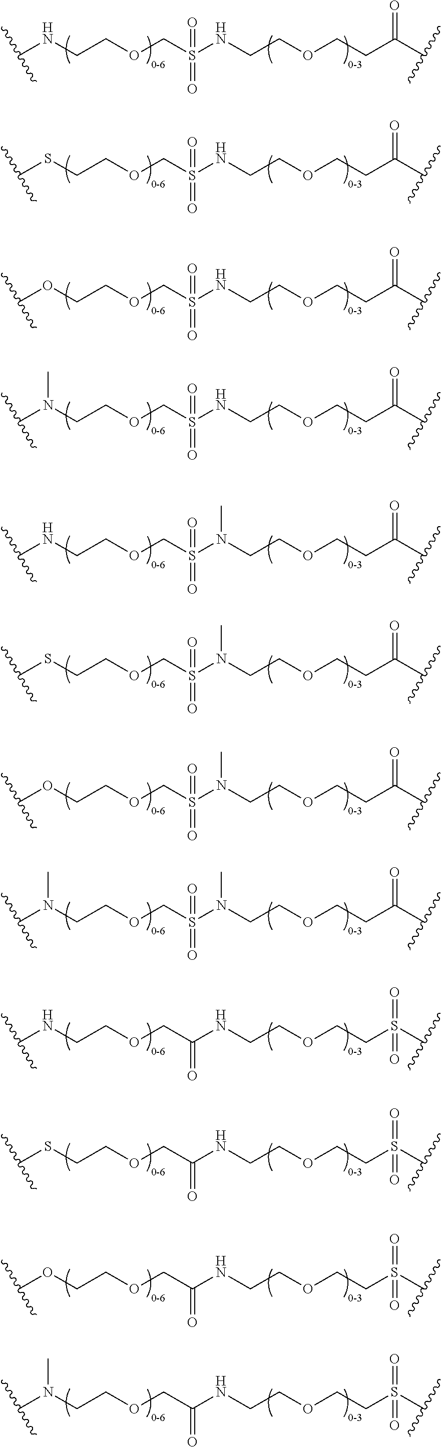

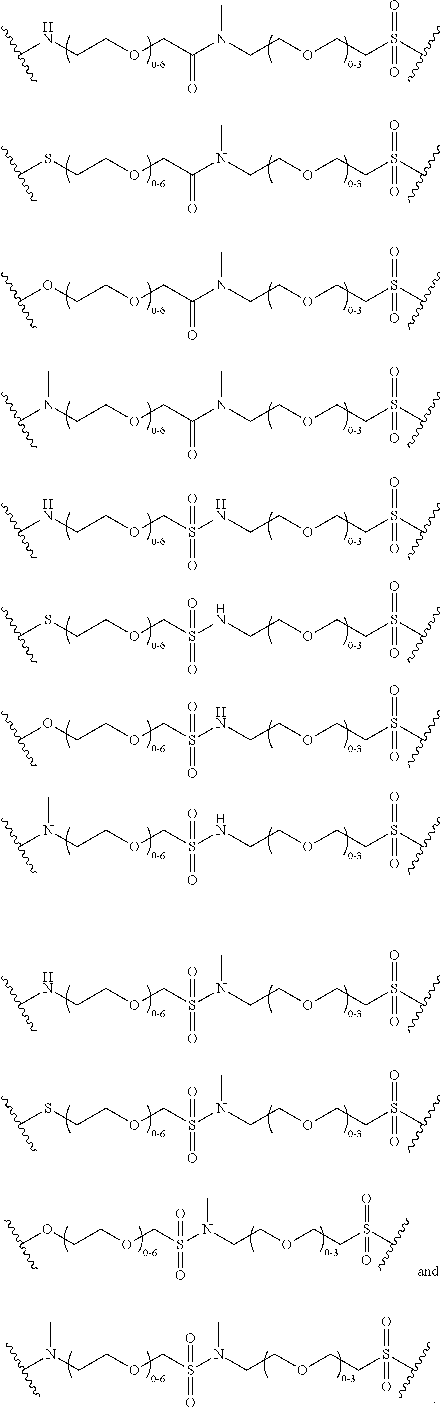

In certain embodiments Linker^{C} is selected from:

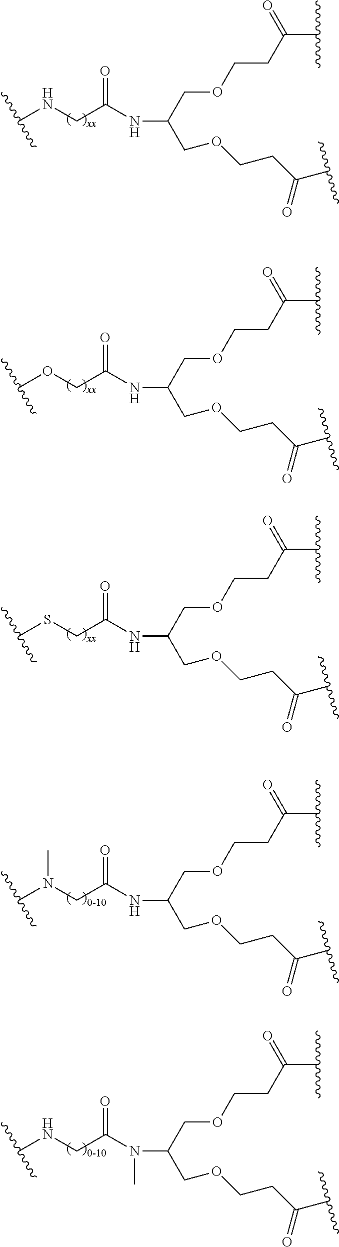

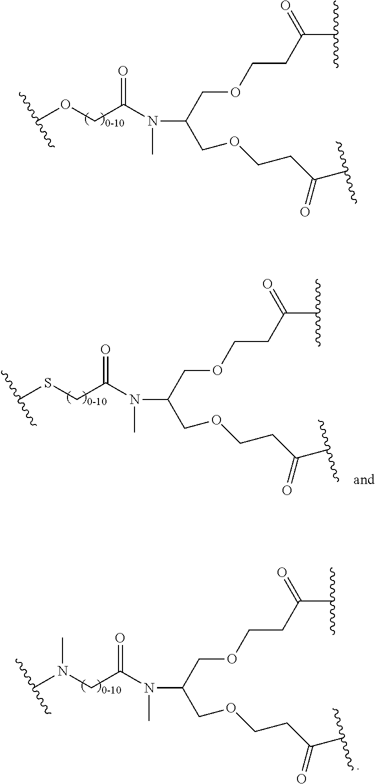

In certain embodiments Linker^{C} is selected from:

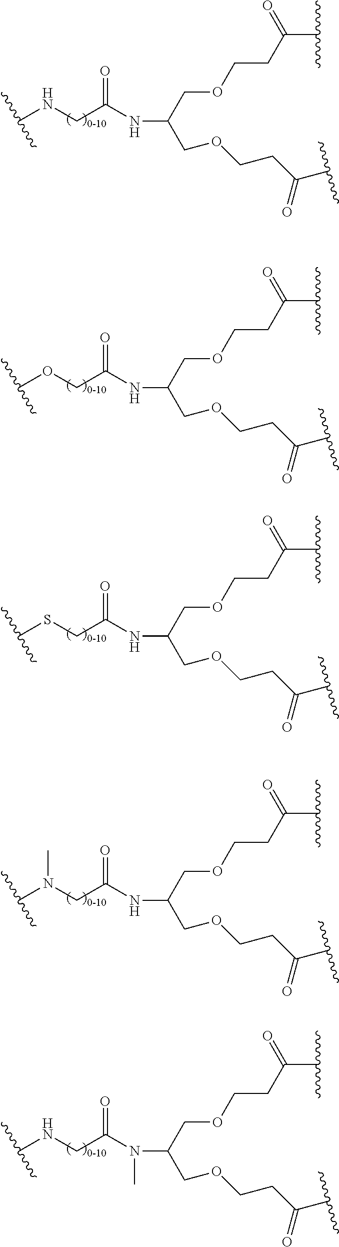

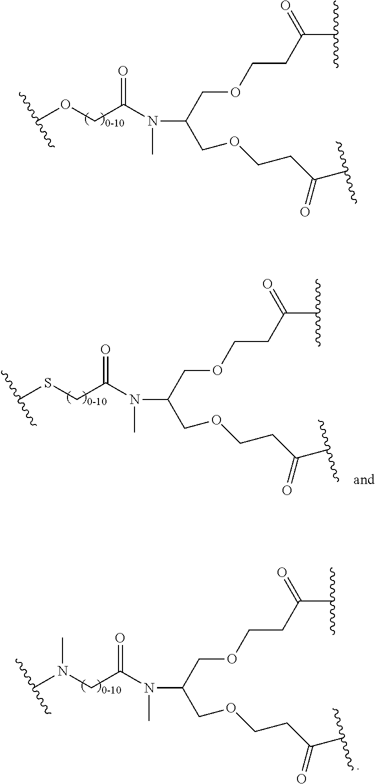

In certain embodiments Linker^{C} is selected from:

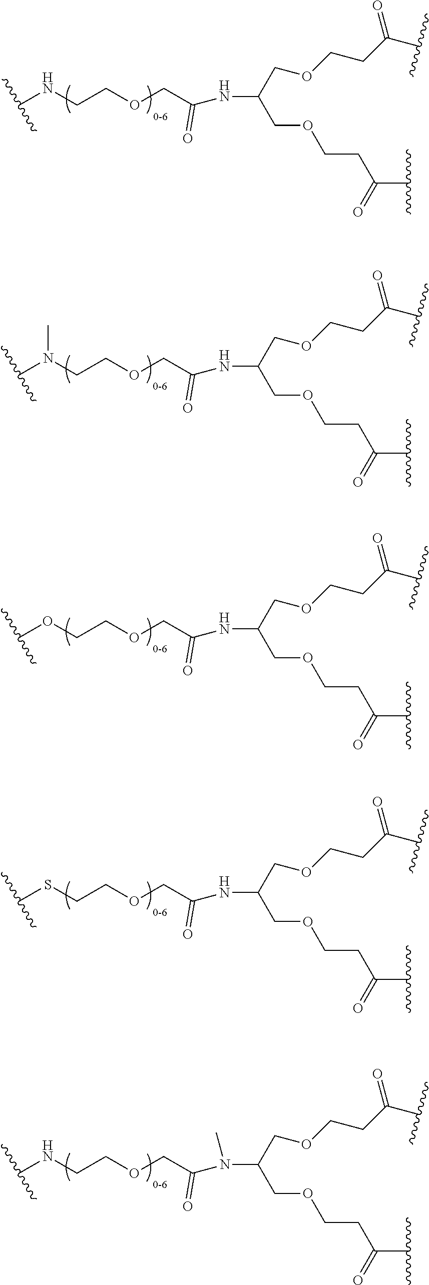

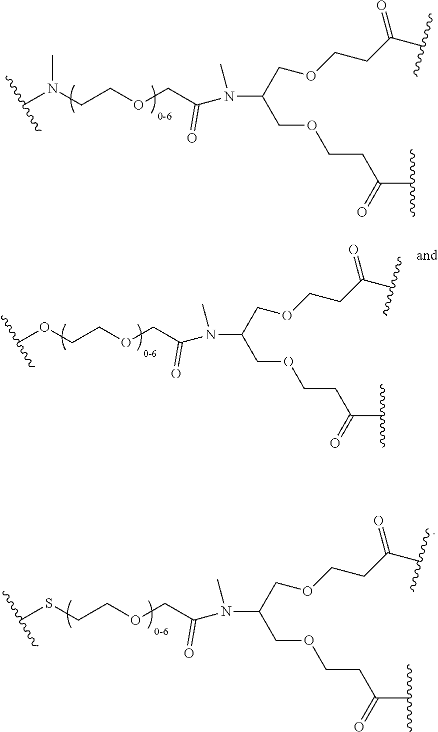

In certain embodiments Linker^{C} is selected from:

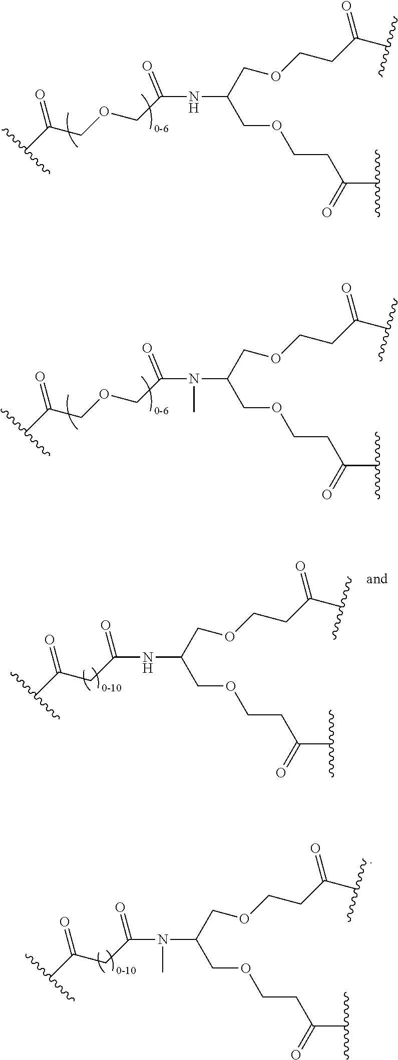

In certain embodiments Linker^{C} is selected from:

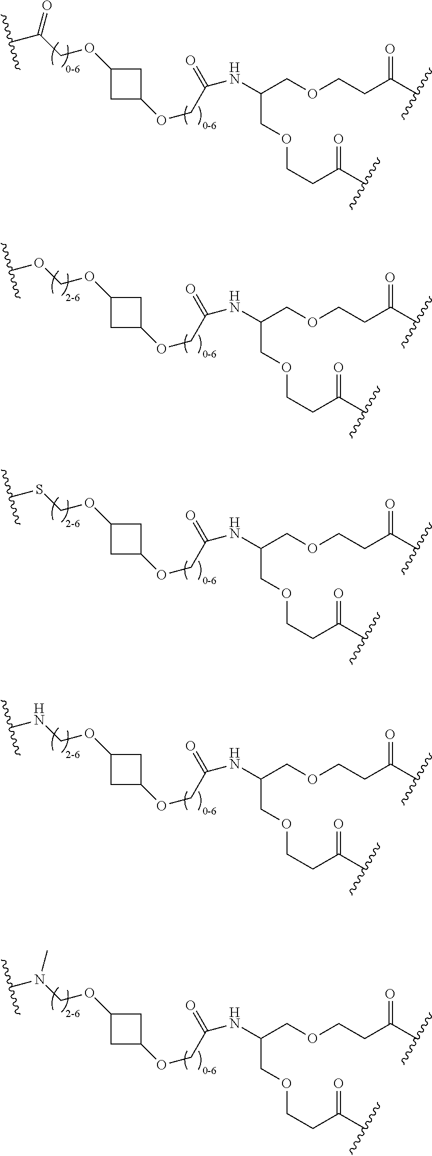

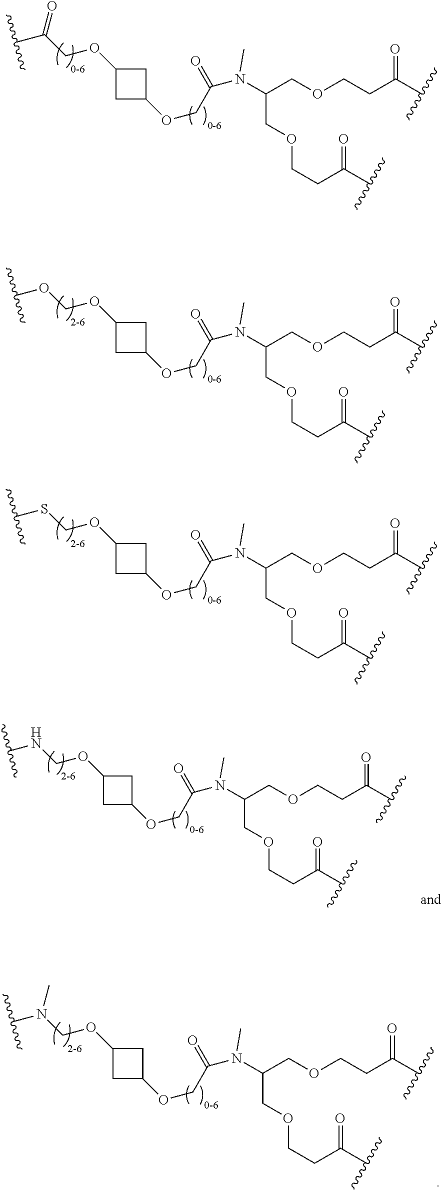

In certain embodiments Linker^{C} is selected from:

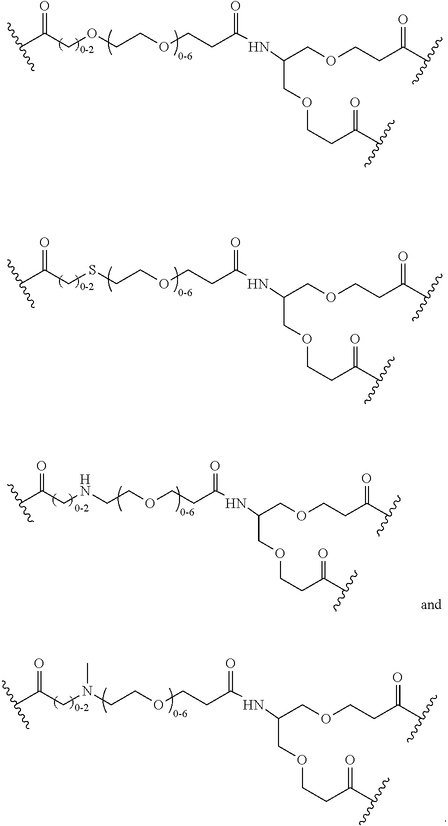

In certain embodiments Linker^{C} is selected from:

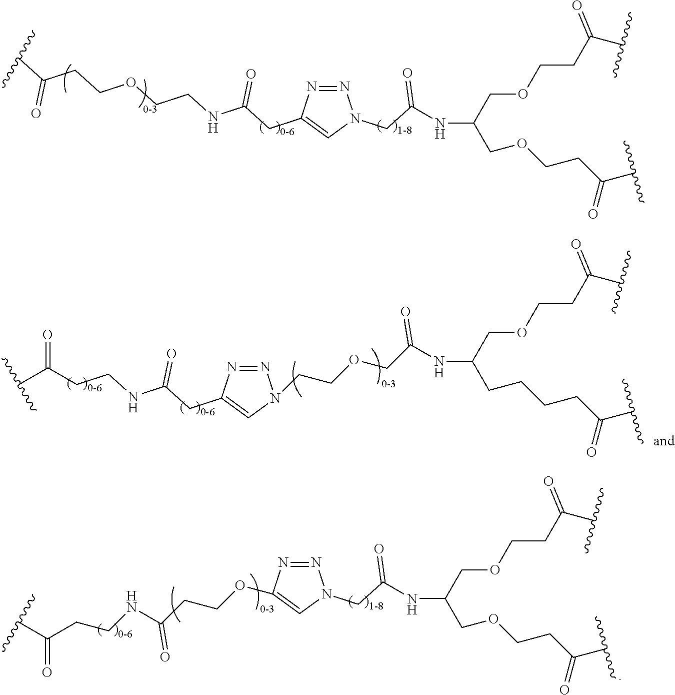

In certain embodiments Linker^{C} is selected from:

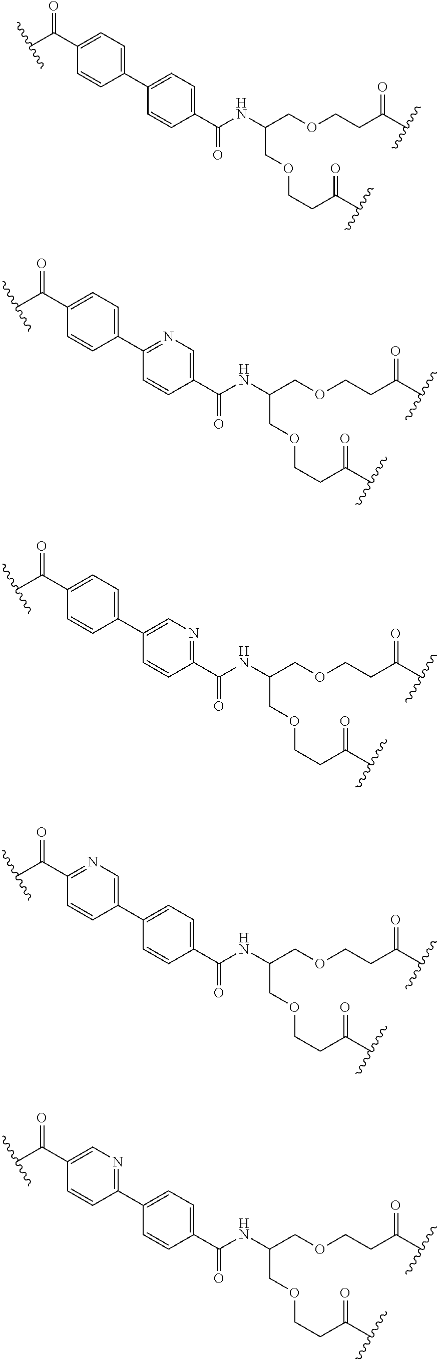 [SMILES: *C(=O)CCOCC(COCCC(*)=O)NC(=O)c1ccc(-c2ccc(C(*)=O)cc2)cc1 ; *C(=O)CCOCC(COCCC(*)=O)NC(=O)c1ccc(-c2ccc(C(*)=O)cc2)nc1 ; *C(=O)CCOCC(COCCC(*)=O)NC(=O)c1ccc(-c2ccc(C(*)=O)cc2)cn1 ; *C(=O)CCOCC(COCCC(*)=O)NC(=O)c1ccc(-c2ccc(C(*)=O)nc2)cc1 ; *C(=O)CCOCC(COCCC(*)=O)NC(=O)c1ccc(-c2ccc(C(*)=O)cn2)cc1]

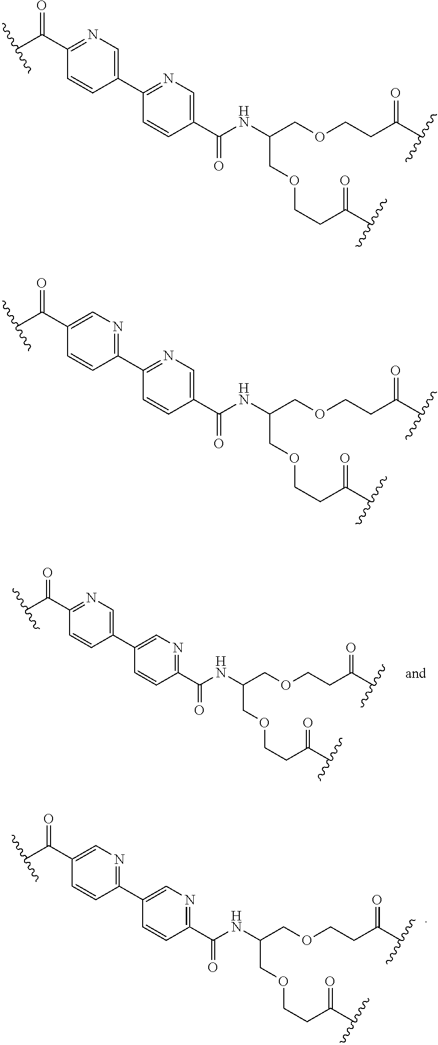 [SMILES: *C(=O)CCOCC(COCCC(*)=O)NC(=O)c1ccc(-c2ccc(C(*)=O)nc2)nc1 ; *C(=O)CCOCC(COCCC(*)=O)NC(=O)c1ccc(-c2ccc(C(*)=O)cn2)nc1 ; *C(=O)CCOCC(COCCC(*)=O)NC(=O)c1ccc(-c2ccc(C(*)=O)nc2)cn1 ; *C(=O)CCOCC(COCCC(*)=O)NC(=O)c1ccc(-c2ccc(C(*)=O)cn2)cn1]

In certain embodiments Linker^{C} is selected from:

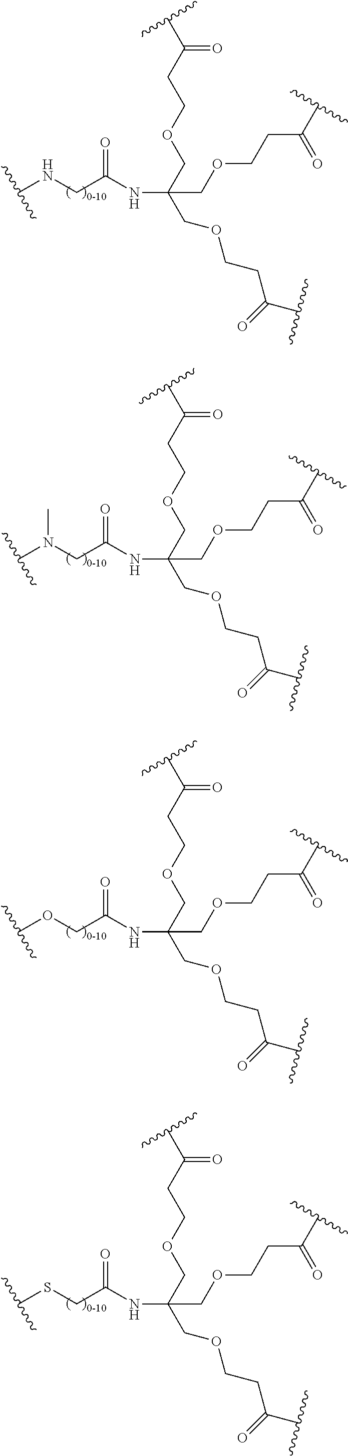

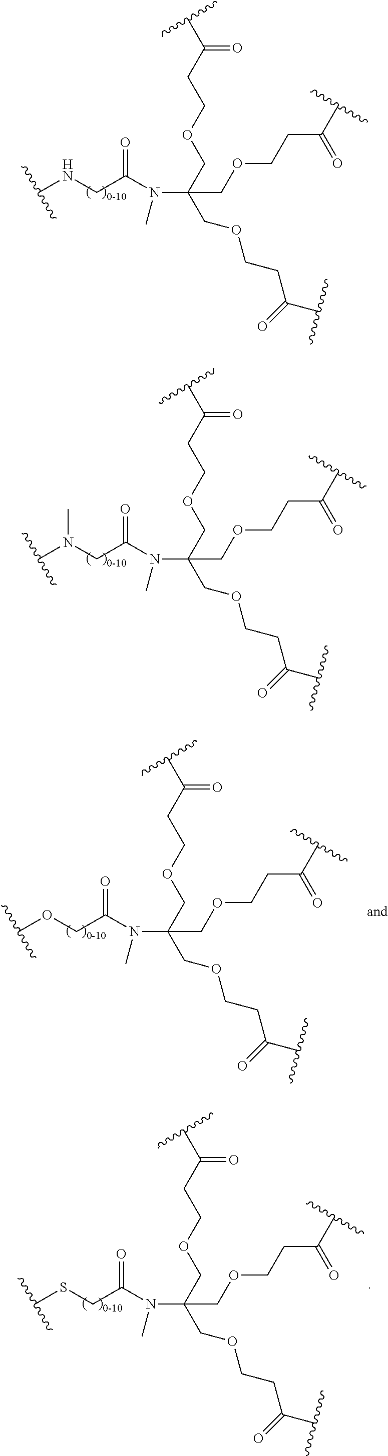

In certain embodiments Linker^{D} is selected from:

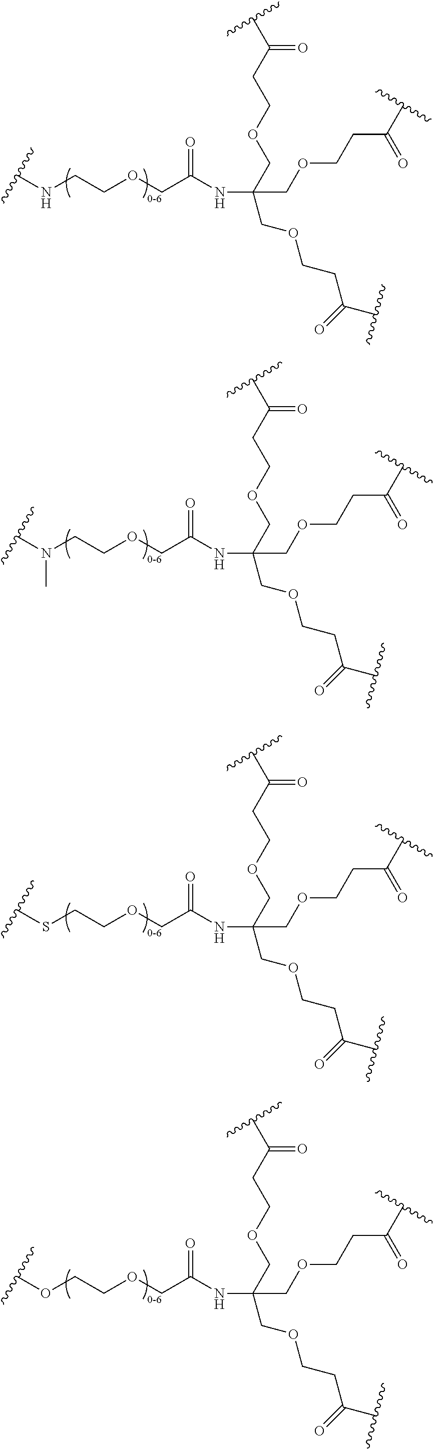

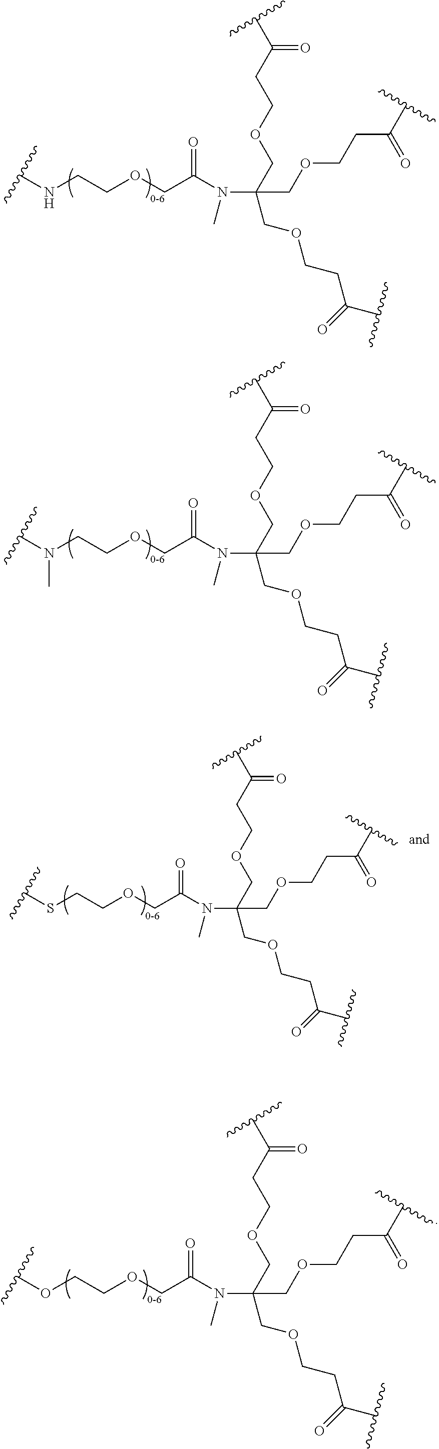

In certain embodiments Linker^{D} is selected from:

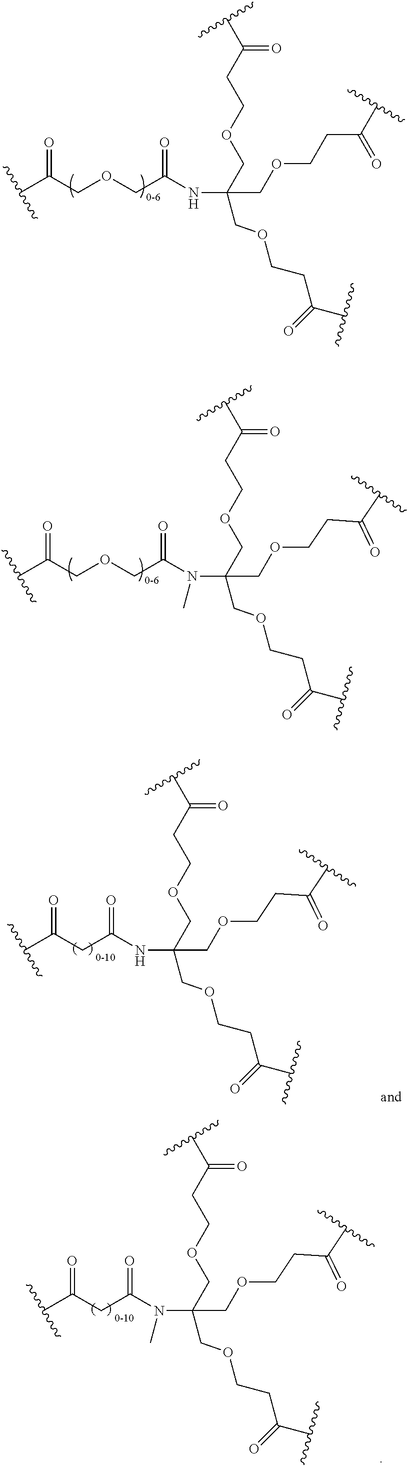

In certain embodiments Linker^{D} is selected from:

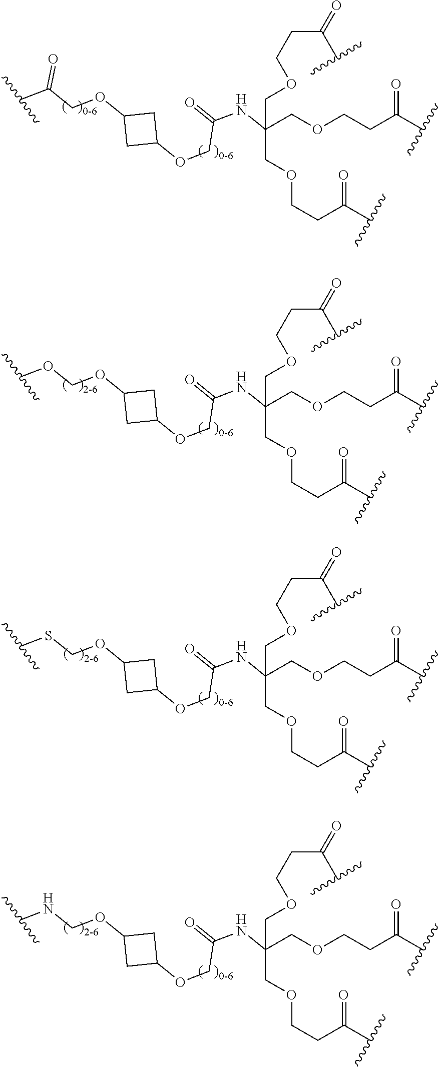

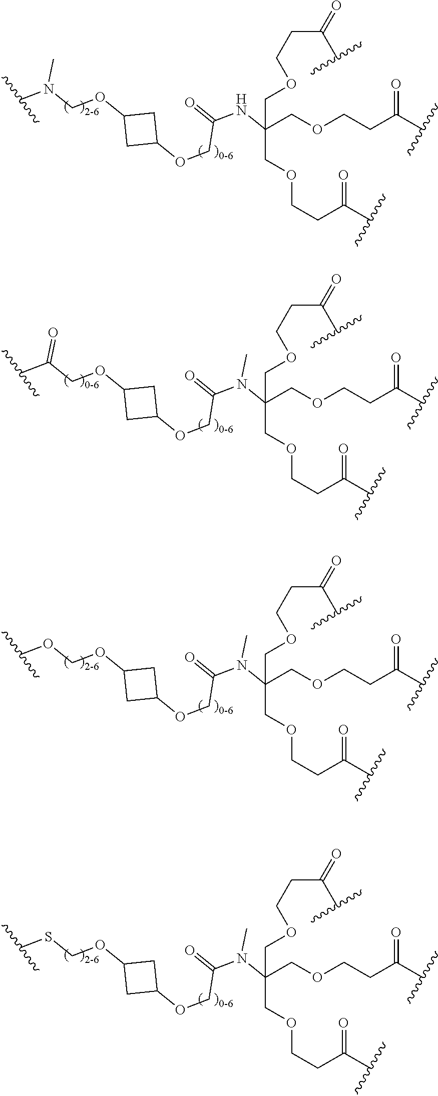

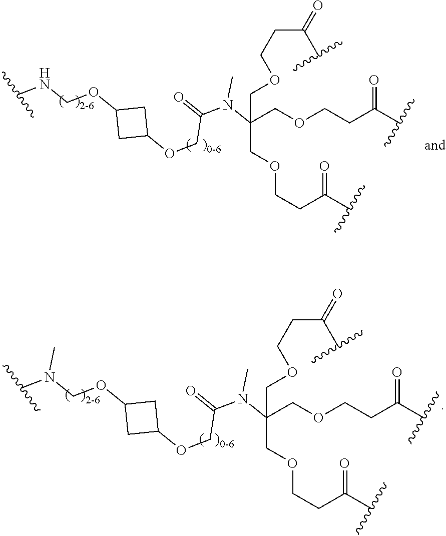

In certain embodiments Linker^{D} is selected from:

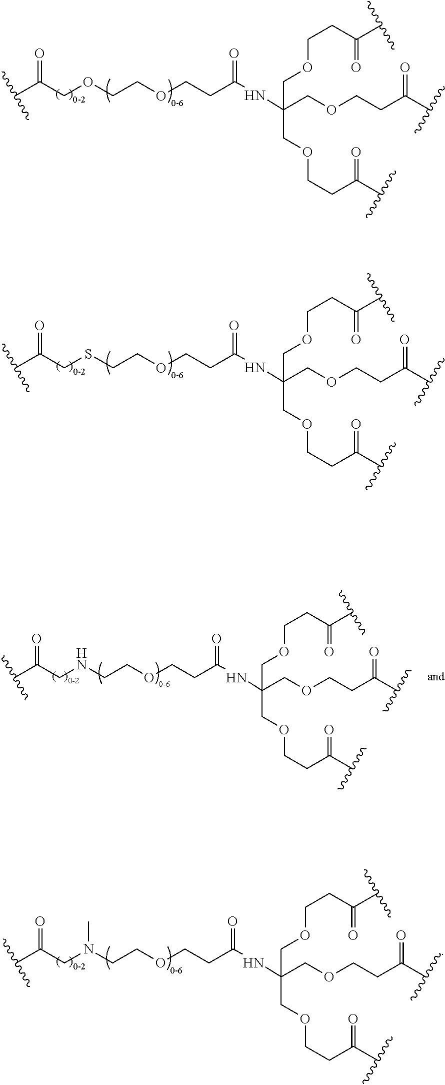

In certain embodiments Linker^{D} is selected from:

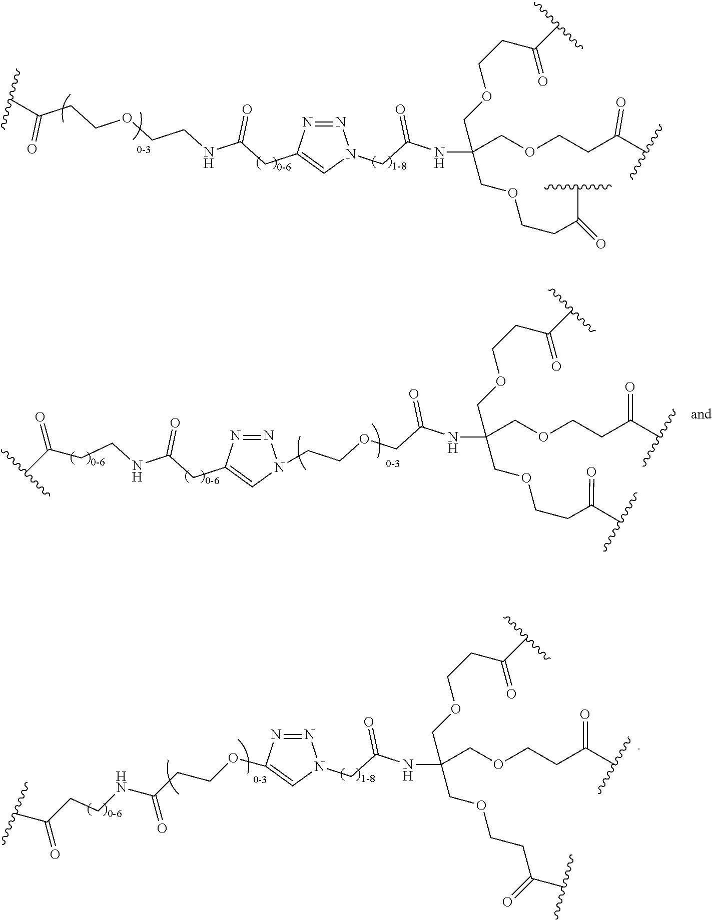

In certain embodiments Linker^{D} is selected from:

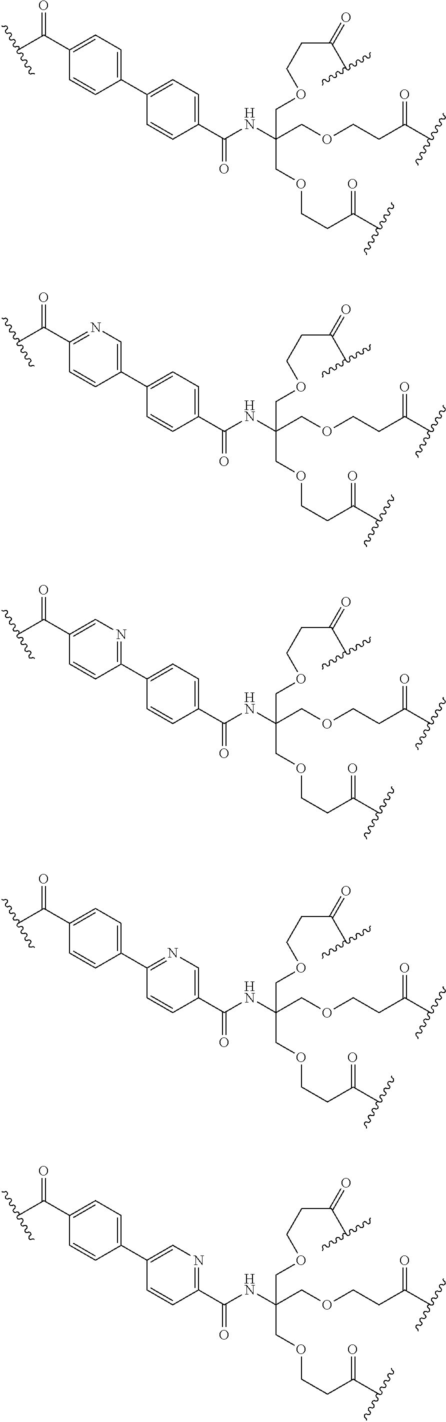 [SMILES: *C(=O)CCOCC(COCCC(*)=O)(COCCC(*)=O)NC(=O)c1ccc(-c2ccc(C(*)=O)cc2)cc1 ; *C(=O)CCOCC(COCCC(*)=O)(COCCC(*)=O)NC(=O)c1ccc(-c2ccc(C(*)=O)nc2)cc1 ; *C(=O)CCOCC(COCCC(*)=O)(COCCC(*)=O)NC(=O)c1ccc(-c2ccc(C(*)=O)cn2)cc1 ; *C(=O)CCOCC(COCCC(*)=O)(COCCC(*)=O)NC(=O)c1ccc(-c2ccc(C(*)=O)cc2)nc1 ; *C(=O)CCOCC(COCCC(*)=O)(COCCC(*)=O)NC(=O)c1ccc(-c2ccc(C(*)=O)cc2)cn1]

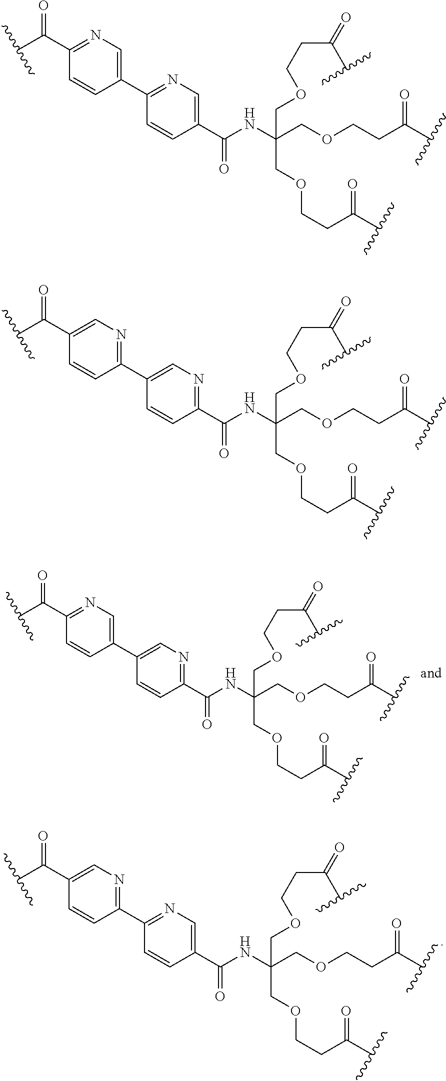 [SMILES: *C(=O)CCOCC(COCCC(*)=O)(COCCC(*)=O)NC(=O)c1ccc(-c2ccc(C(*)=O)nc2)nc1 ; *C(=O)CCOCC(COCCC(*)=O)(COCCC(*)=O)NC(=O)c1ccc(-c2ccc(C(*)=O)cn2)cn1 ; *C(=O)CCOCC(COCCC(*)=O)(COCCC(*)=O)NC(=O)c1ccc(-c2ccc(C(*)=O)nc2)cn1 ; *C(=O)CCOCC(COCCC(*)=O)(COCCC(*)=O)NC(=O)c1ccc(-c2ccc(C(*)=O)cn2)nc1]

In certain embodiments, the Linker^{A} is selected from

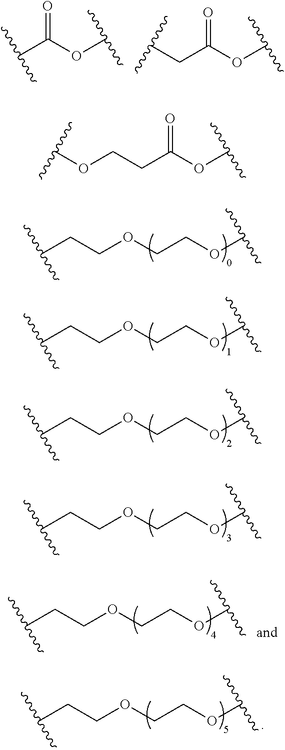

In certain embodiments, the Linker^{A} is selected from

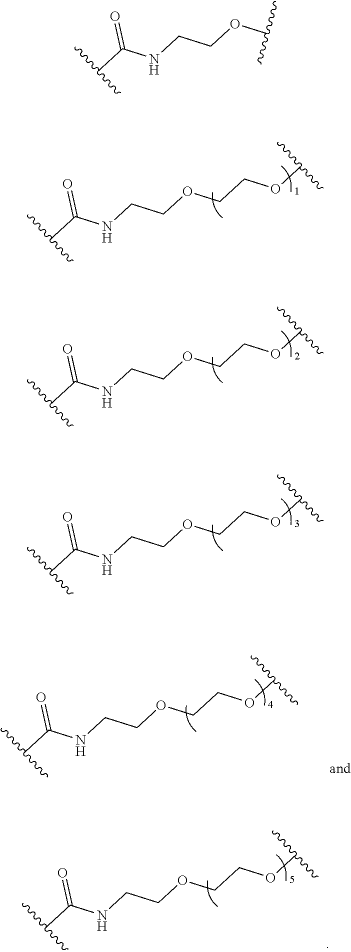

In certain embodiments, the Linker^{A} is selected from

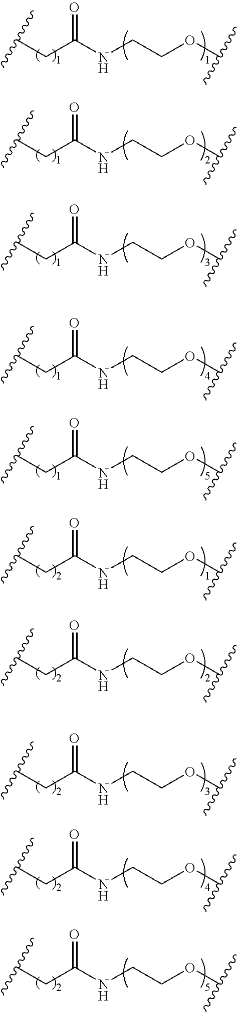

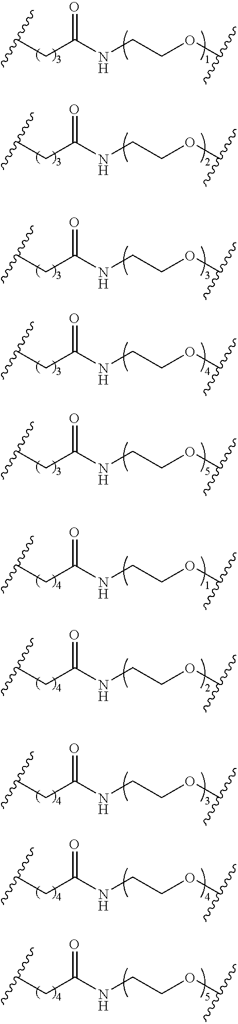

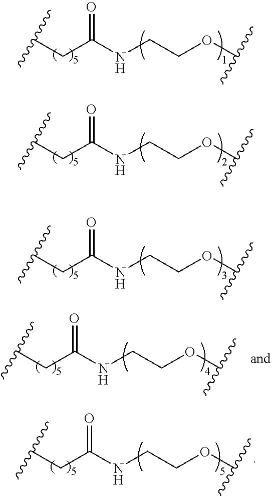

In certain embodiments, the Linker^{A} is selected from

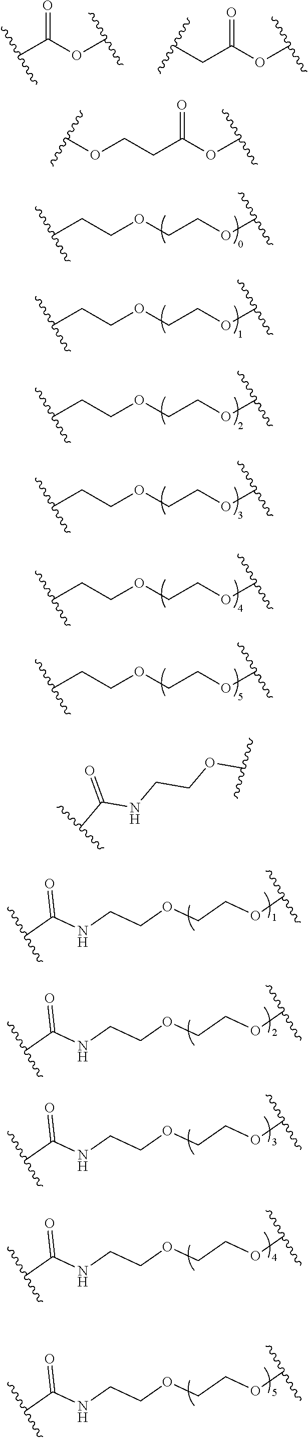

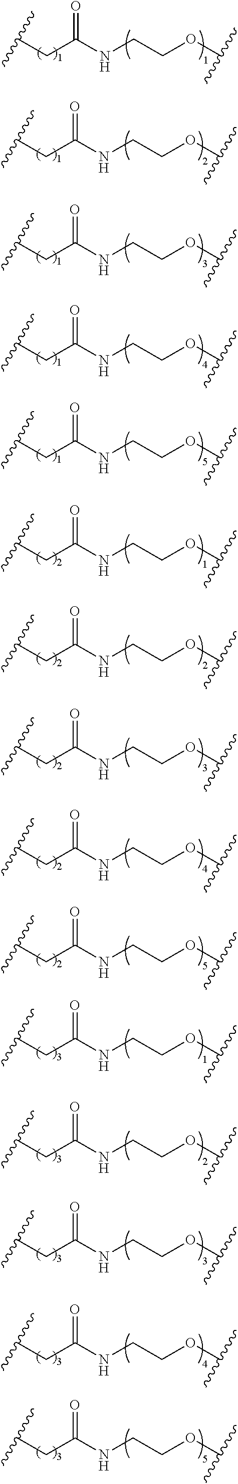

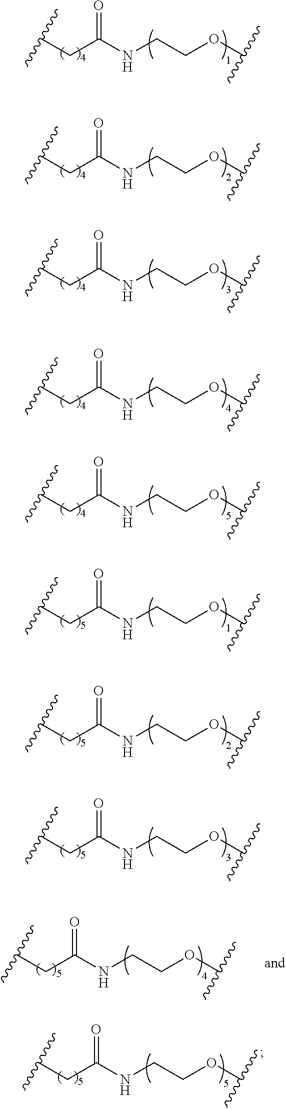

wherein each is optionally substituted with 1, 2, 3, or 4 substituents substituent selected from R²¹.

In certain embodiments Linker^{A} is selected from:

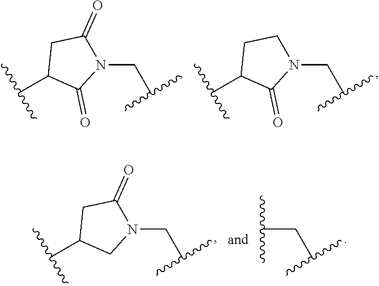

In certain embodiments Linker^{A} is selected from:

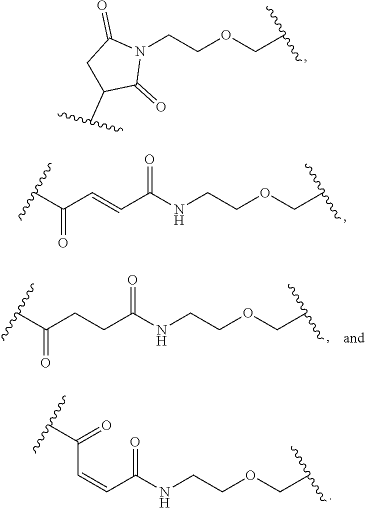 [SMILES: *COCCN1C(=O)CC(*)C1=O ; *COCCNC(=O)/C=C/C(*)=O ; *COCCNC(=O)CCC(*)=O ; *COCCNC(=O)/C=C\C(*)=O]

In certain embodiments Linker^{A} is selected from:

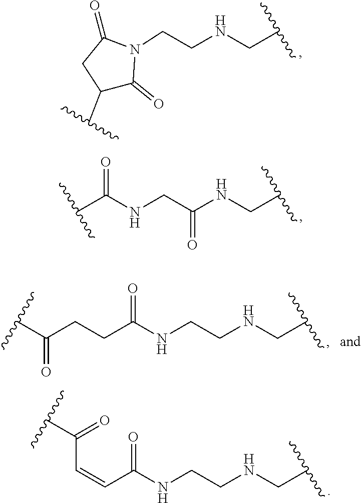

In certain embodiments Linker^{A} is selected from:

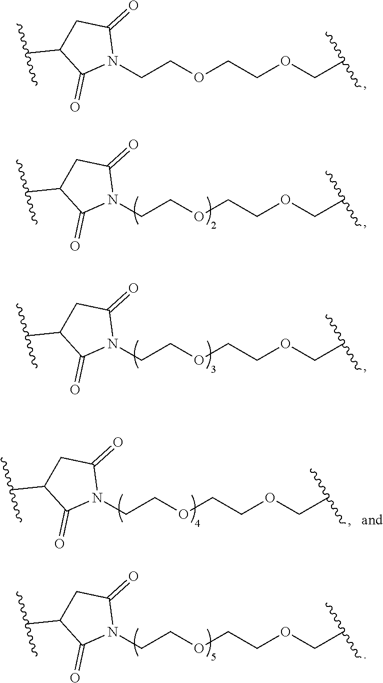

In certain embodiments Linker^{A} is selected from:

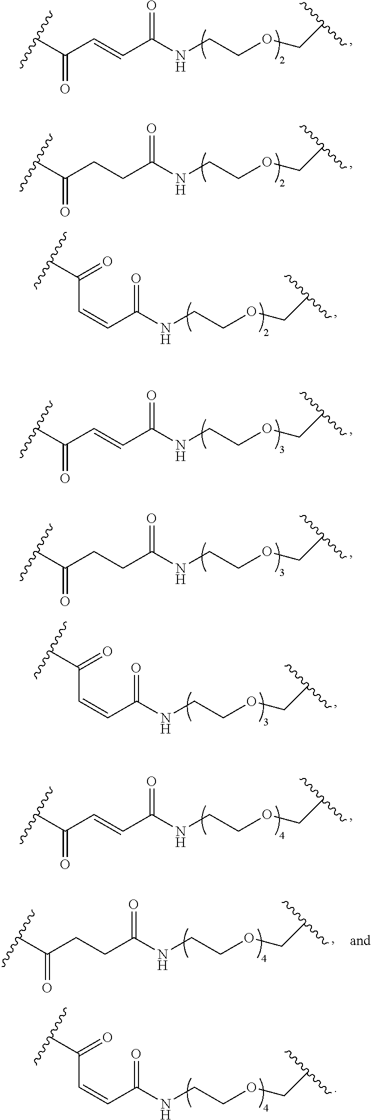

In certain embodiments Linker^{A} is selected from:

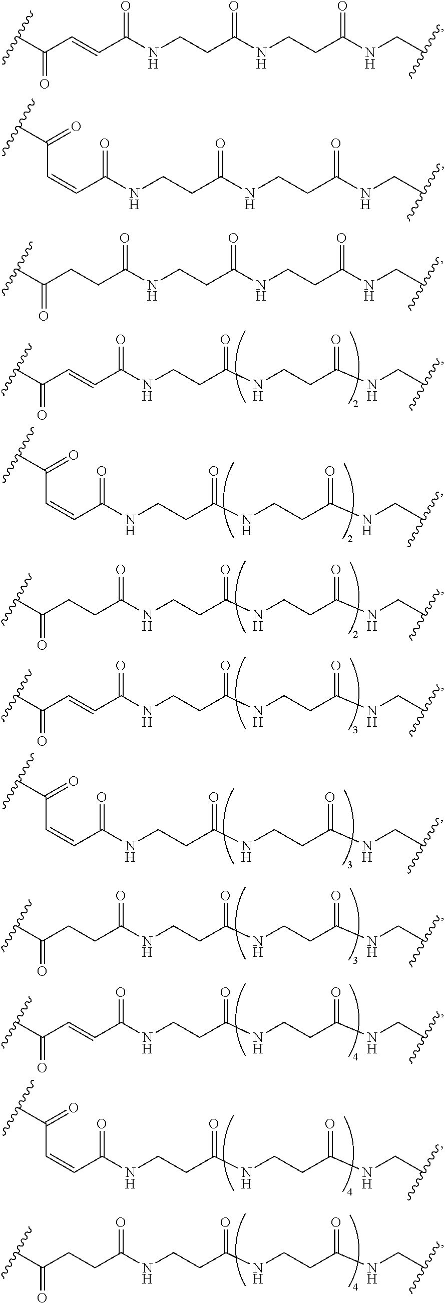

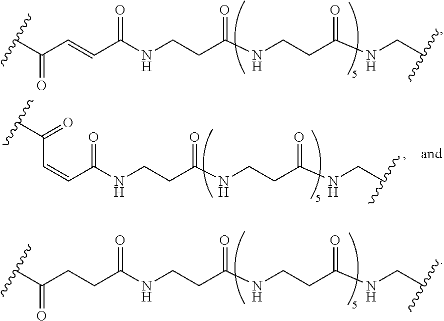

In certain embodiments Linker^{A} is selected from:

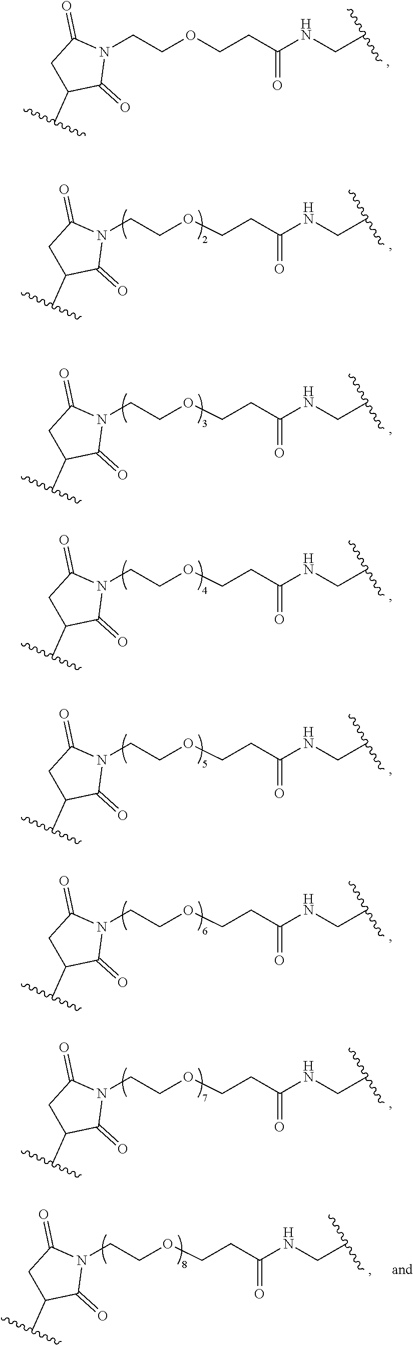

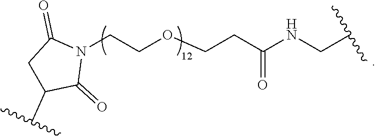

In certain embodiments Linker^{A} is selected from:

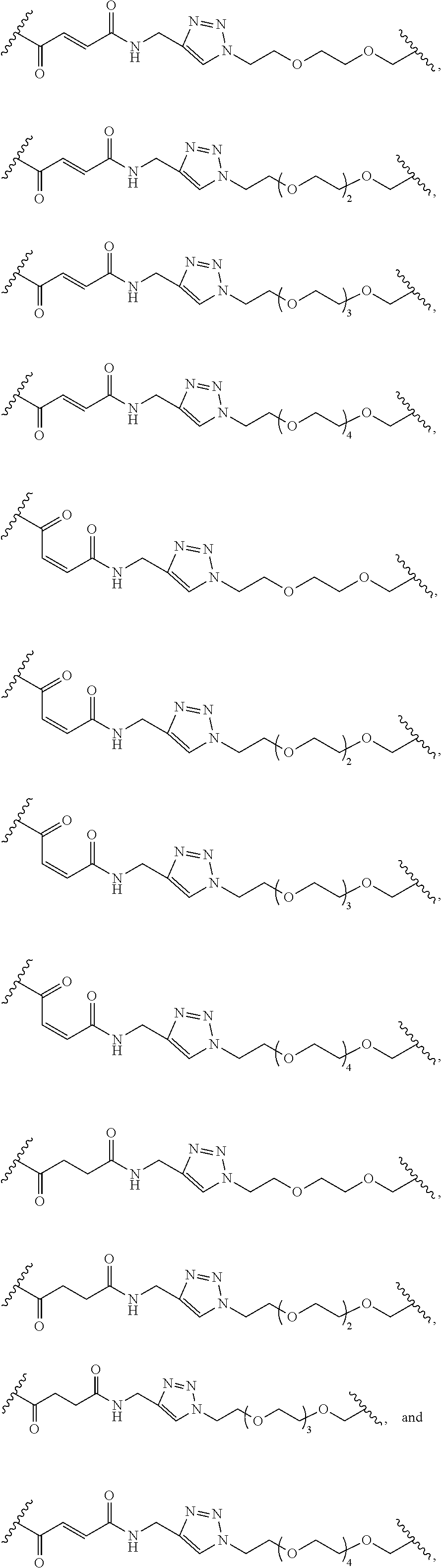

In certain embodiments Linker^{A} is selected from:

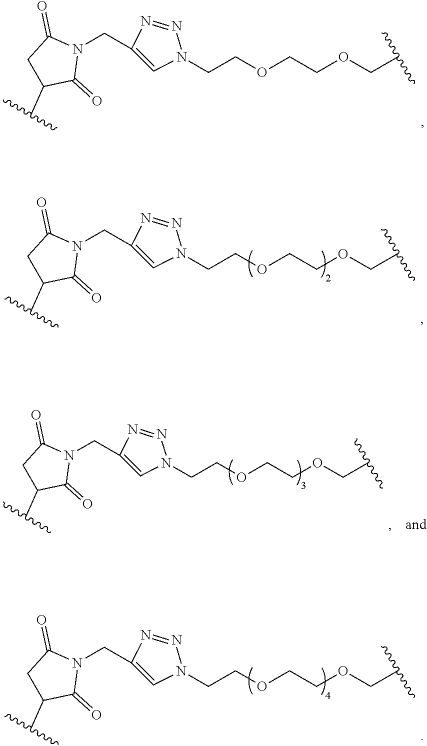

In certain embodiments Linker is selected from:

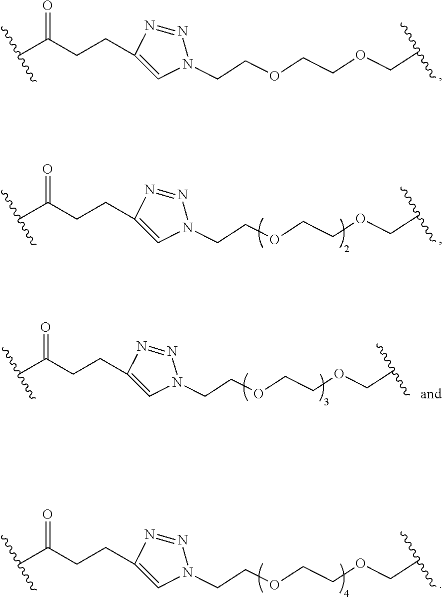

In certain embodiments Linker^{A} is selected from:

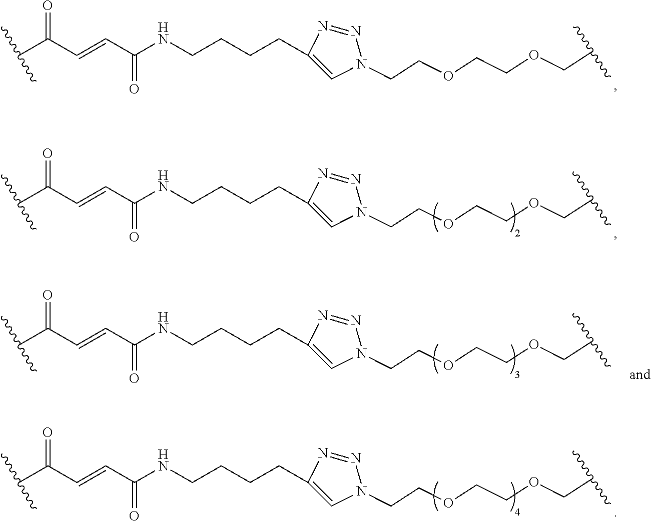

In certain embodiments Linker^{A} is selected from:

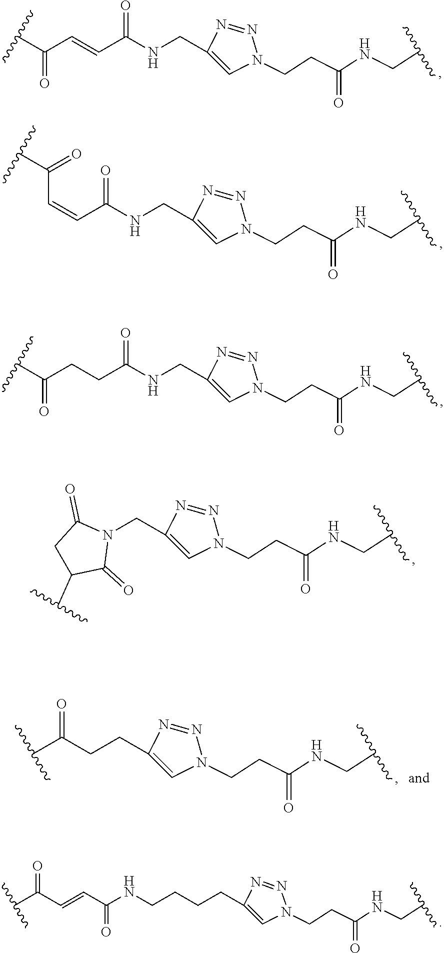 [SMILES: *CNC(=O)CCn1cc(CNC(=O)/C=C/C(*)=O)nn1 ; *CNC(=O)CCn1cc(CNC(=O)/C=C\C(*)=O)nn1 ; *CNC(=O)CCn1cc(CNC(=O)CCC(*)=O)nn1 ; *CNC(=O)CCn1cc(CN2C(=O)CC(*)C2=O)nn1 ; *CNC(=O)CCn1cc(CCC(*)=O)nn1 ; *CNC(=O)CCn1cc(CCCCNC(=O)/C=C/C(*)=O)nn1]

In certain embodiments Linker^{A} is selected from:

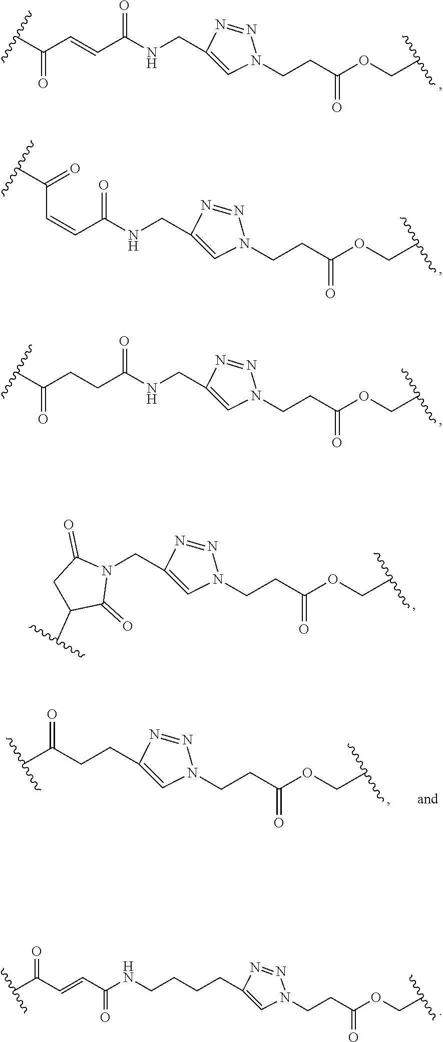 [SMILES: *COC(=O)CCn1cc(CNC(=O)/C=C/C(*)=O)nn1 ; *COC(=O)CCn1cc(CNC(=O)/C=C\C(*)=O)nn1 ; *COC(=O)CCn1cc(CNC(=O)CCC(*)=O)nn1 ; *COC(=O)CCn1cc(CN2C(=O)CC(*)C2=O)nn1 ; *COC(=O)CCn1cc(CCC(*)=O)nn1 ; *COC(=O)CCn1cc(CCCCNC(=O)/C=C/C(*)=O)nn1]

In certain embodiments, the Linker^{B} is selected from

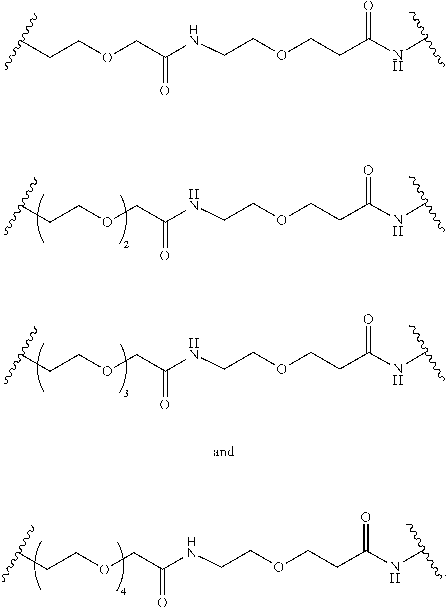

In certain embodiments, the Linker^{B} is selected from

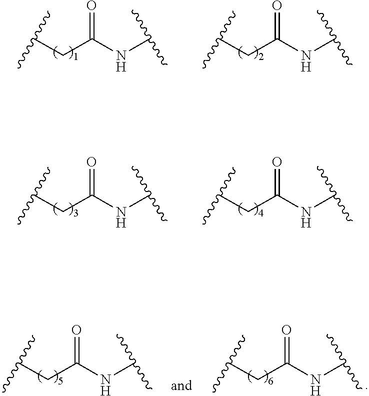

In certain embodiments, the Linker^{B} is selected from

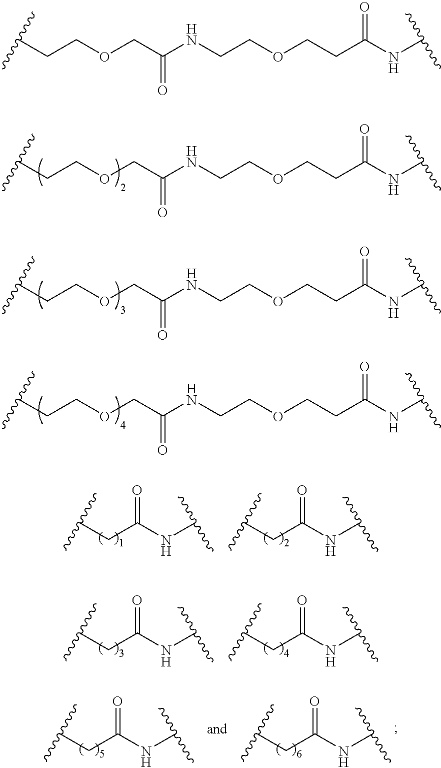

wherein each is optionally substituted with 1, 2, 3, or 4 substituents substituent selected from R²¹.

In certain embodiments Linker^{B} is selected from:

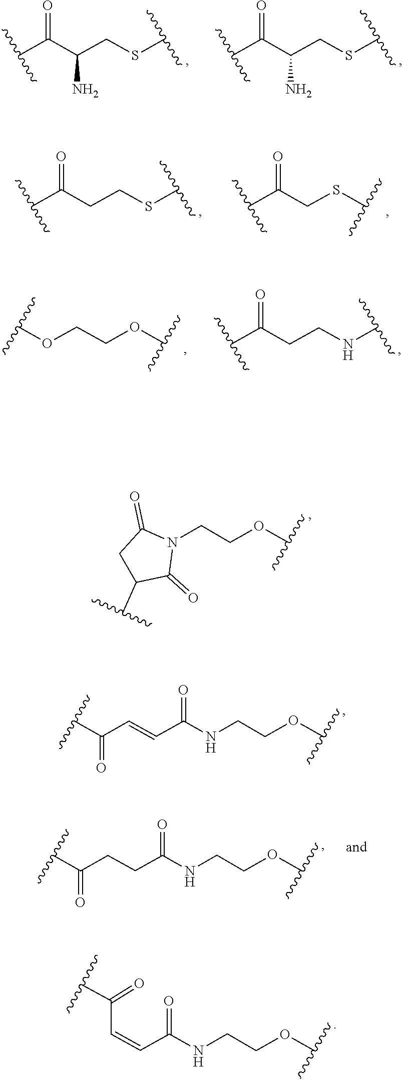

In certain embodiments Linker^{B} is selected from:

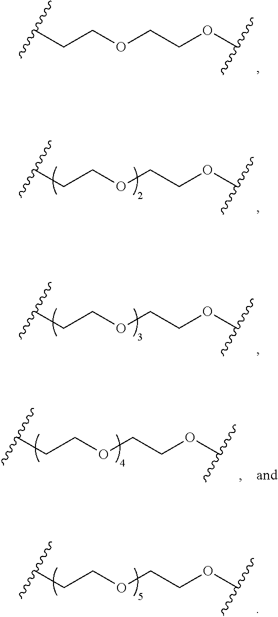

In certain embodiments Linker^{B} is selected from:

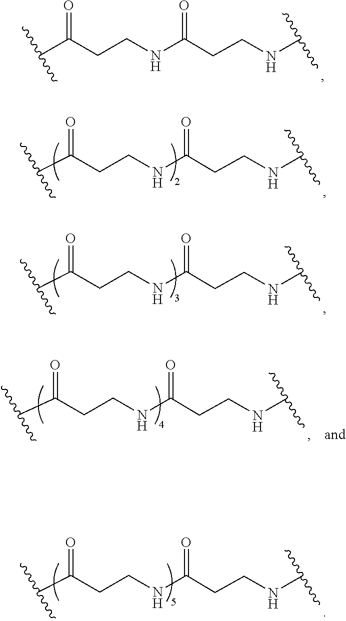

In certain embodiments Linker^{B} is selected from:

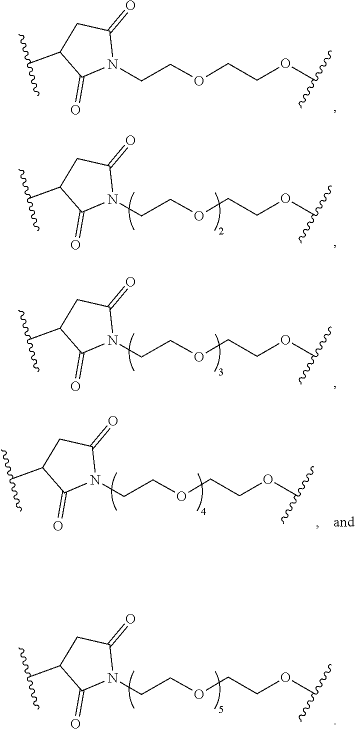

In certain embodiments Linker^{B} is selected from:

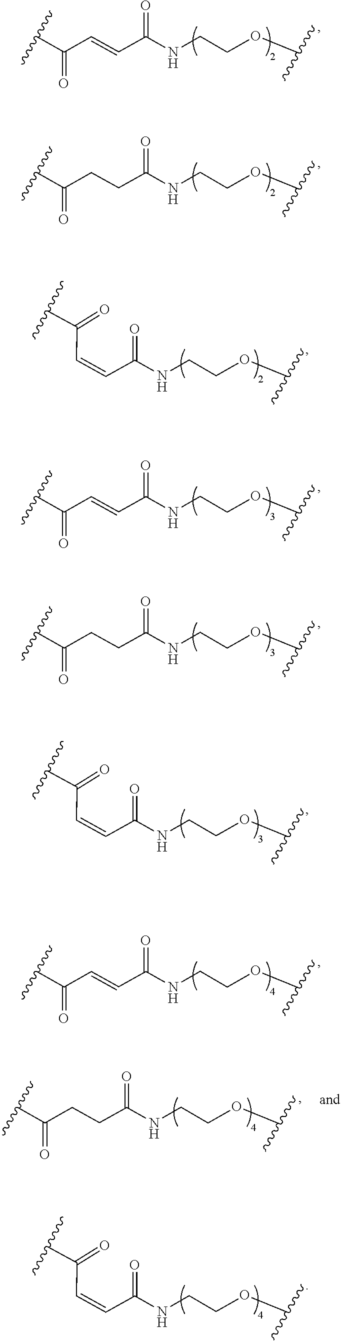

In certain embodiments Linker^{B} is selected from:

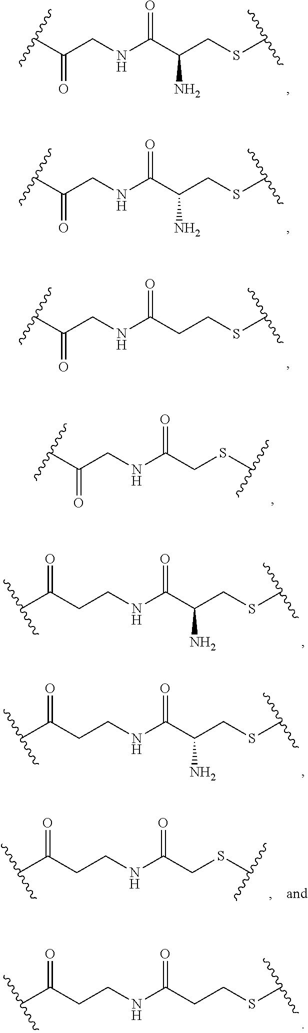

In certain embodiments Linker^{B} is selected from:

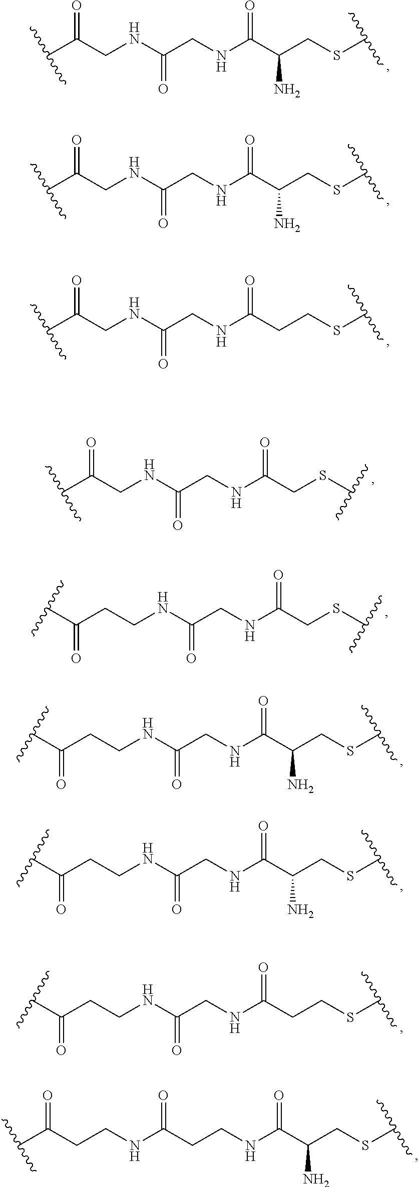

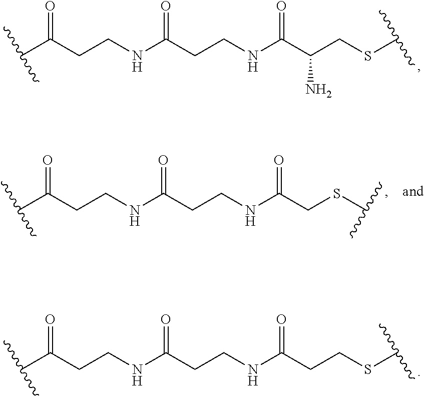 [SMILES: *SC[C@H](N)C(=O)NCCC(=O)NCCC(*)=O ; *SCC(=O)NCCC(=O)NCCC(*)=O ; *SCCC(=O)NCCC(=O)NCCC(*)=O]

In certain embodiments Linker^{B} is selected from:

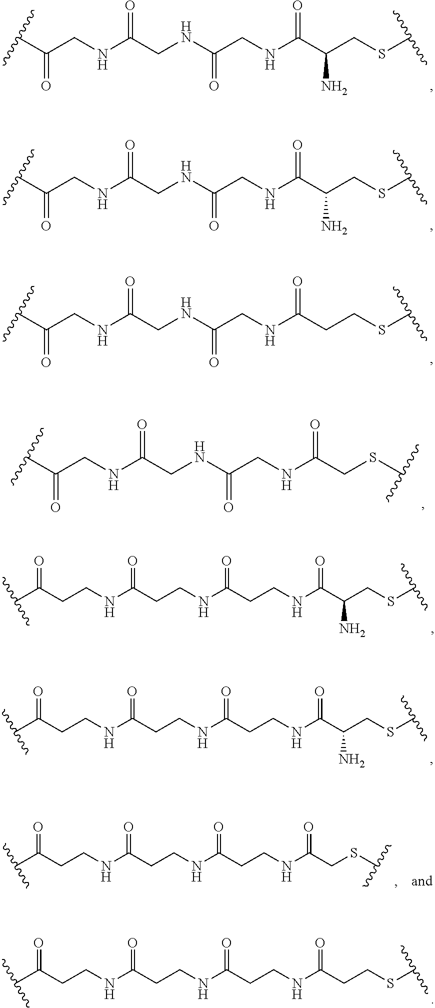

In certain embodiments Linker^{B}-Linker^{A} is selected from:

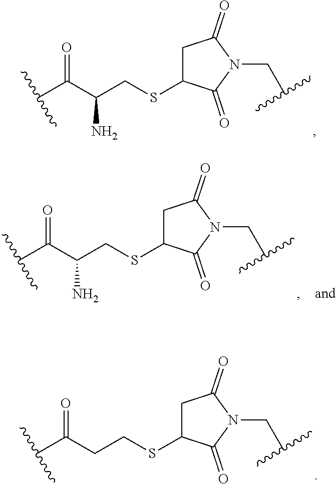 [SMILES: *CN1C(=O)CC(SC[C@@H](N)C(*)=O)C1=O ; *CN1C(=O)CC(SC[C@H](N)C(*)=O)C1=O ; *CN1C(=O)CC(SCCC(*)=O)C1=O]

In certain embodiments Linker^{B}-Linker^{A} is selected from:

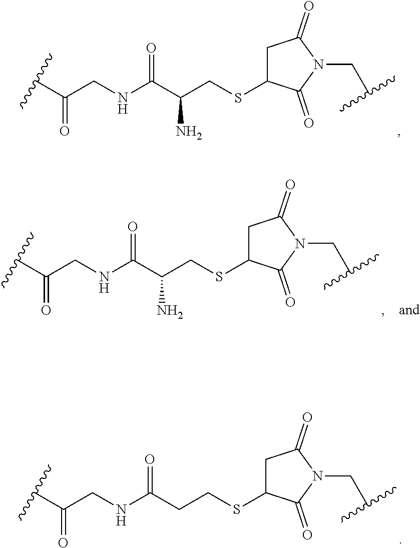 [SMILES: *CN1C(=O)CC(SC[C@@H](N)C(=O)NCC(*)=O)C1=O ; *CN1C(=O)CC(SC[C@H](N)C(=O)NCC(*)=O)C1=O ; *CN1C(=O)CC(SCCC(=O)NCC(*)=O)C1=O]

In certain embodiments, the Linker^{C} is selected from

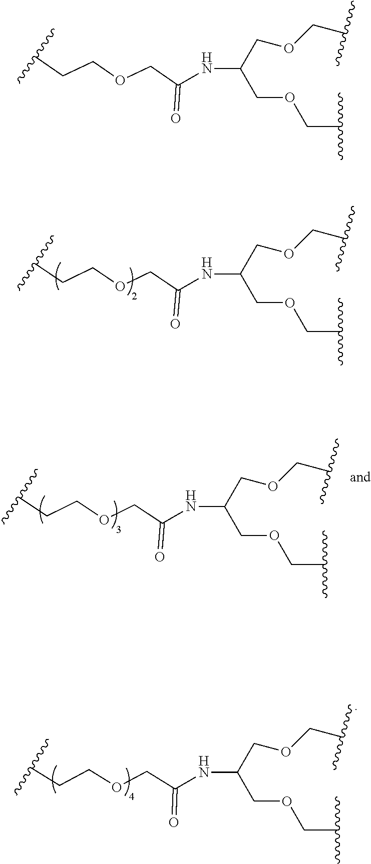

In certain embodiments, the Linker^{C} is selected from

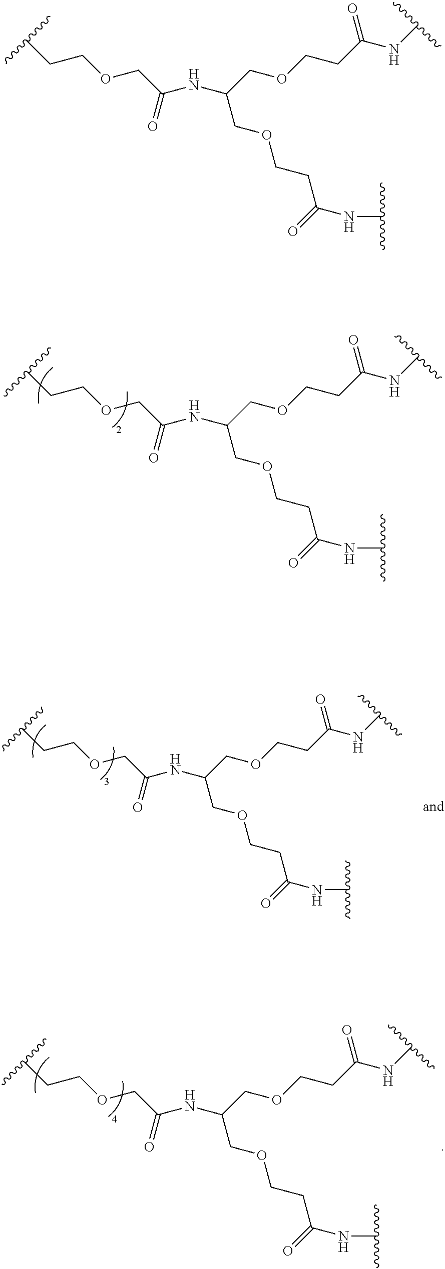

In certain embodiments, the Linker^{C} is selected from

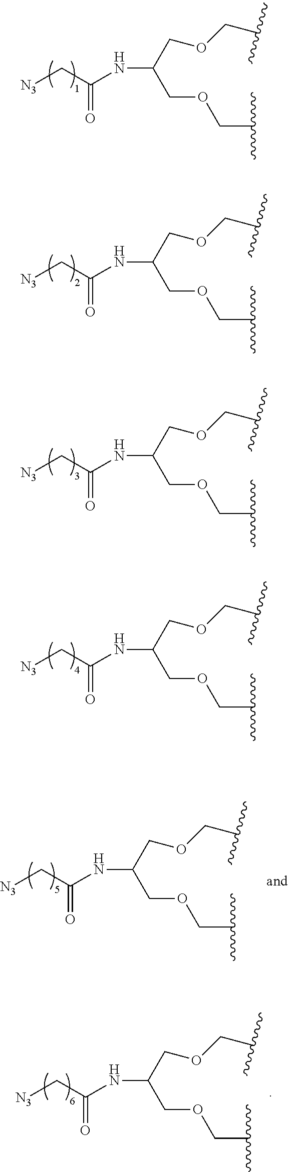

In certain embodiments, the Linker^{C} is selected from

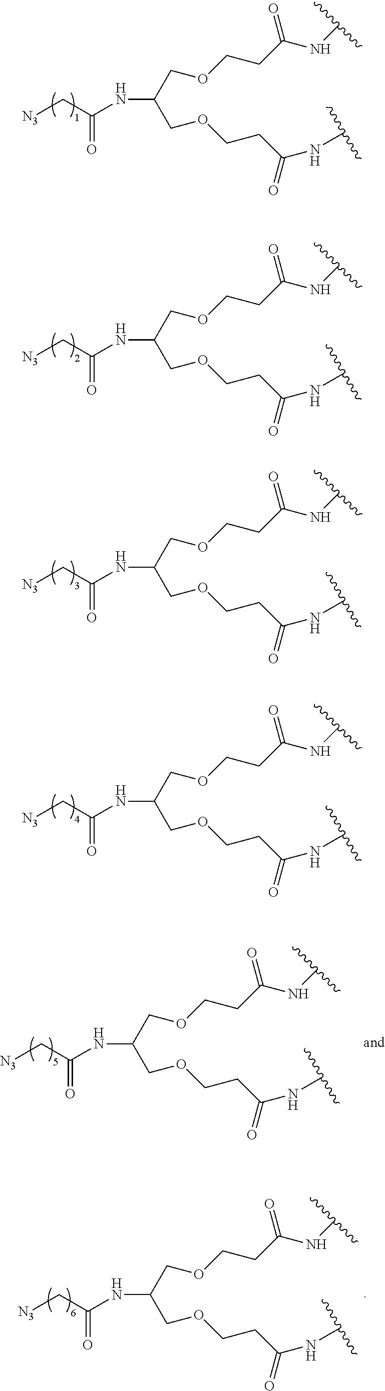

In certain embodiments, the Linker^{C} is selected from

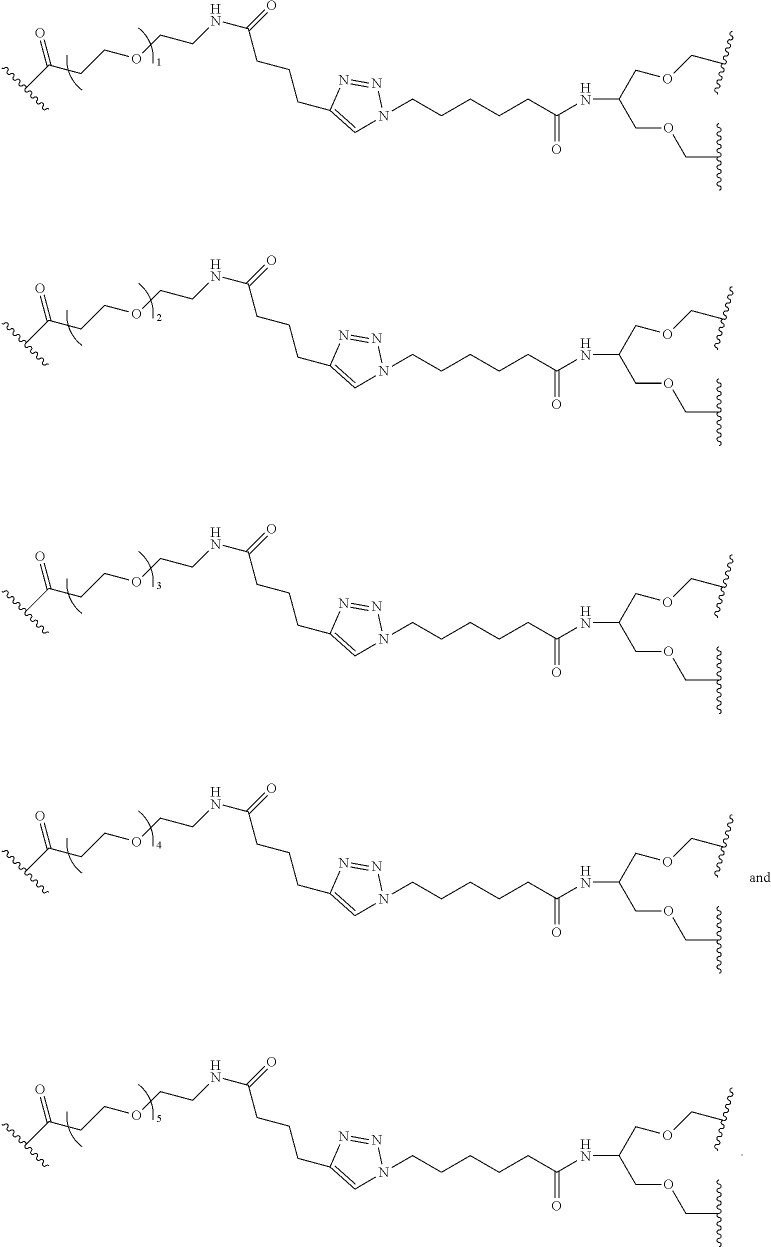

In certain embodiments, the Linker^{C} is selected from

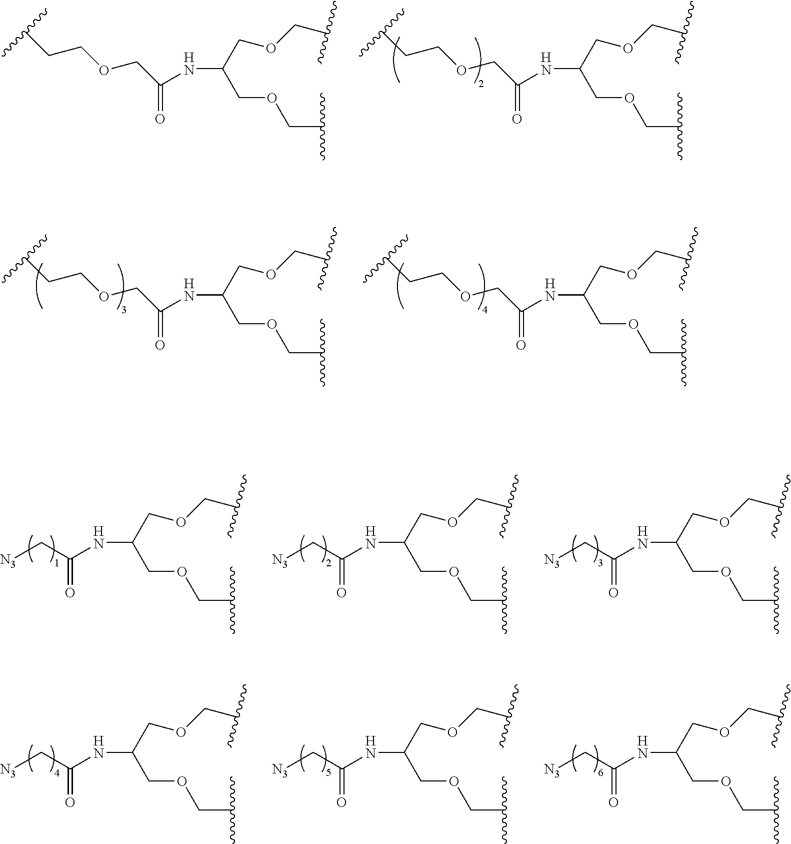

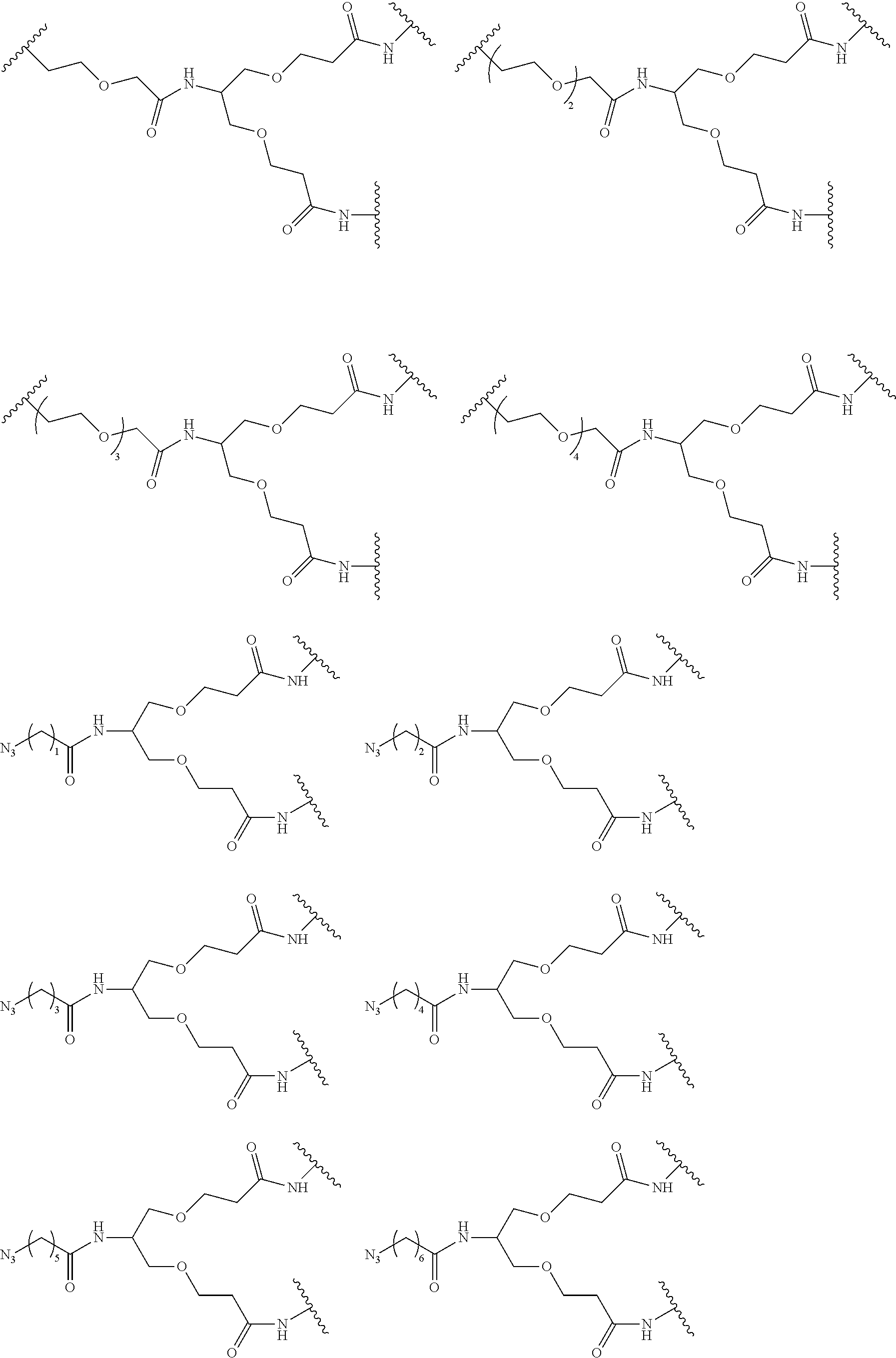

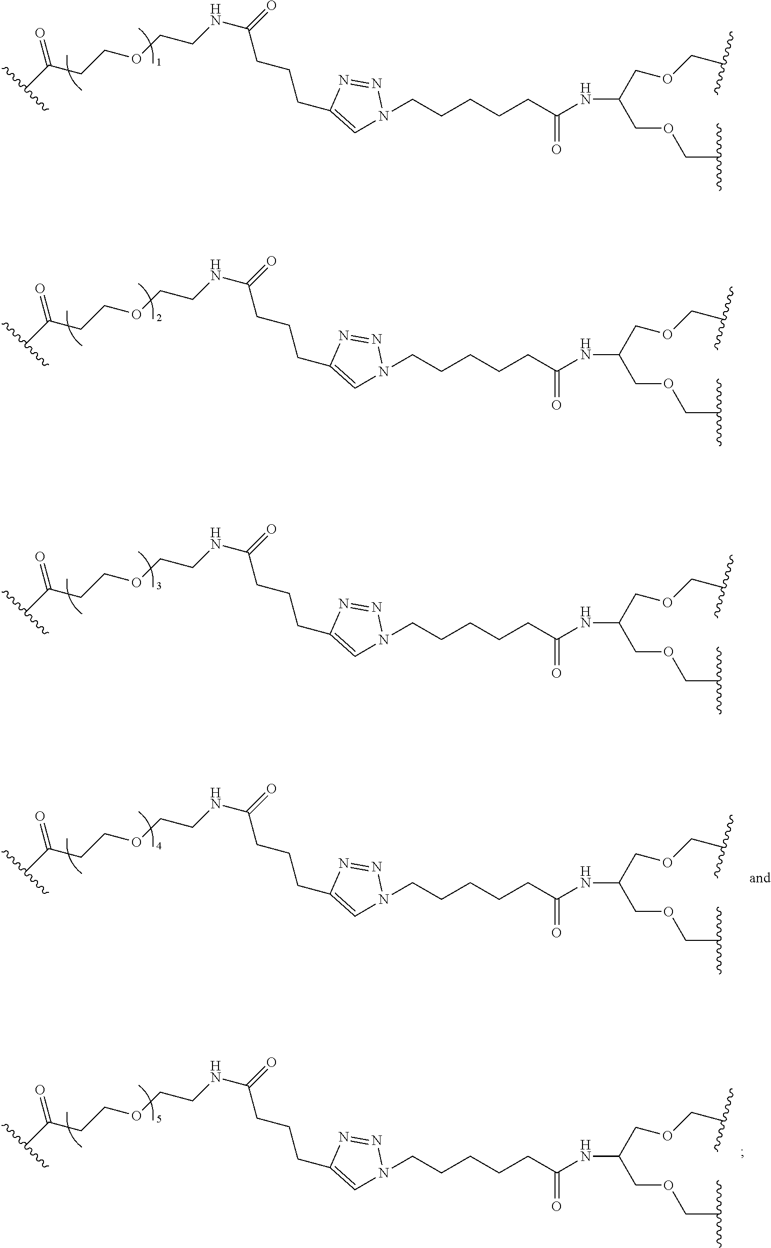

wherein each is optionally substituted with 1, 2, 3, or 4 substituents substituent selected from R²¹.

In certain embodiments Linker^{C} is selected from:

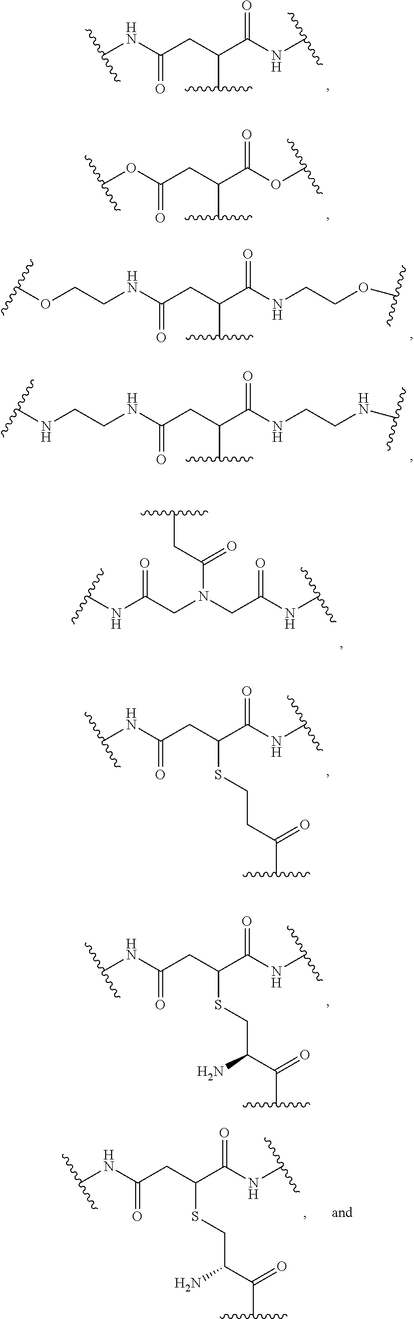

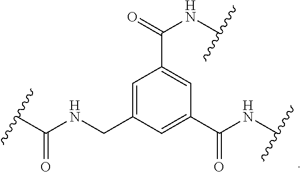 [SMILES: *NC(=O)c1cc(CNC(*)=O)cc(C(=O)N*)c1]

In certain embodiments Linker^{C} is selected from:

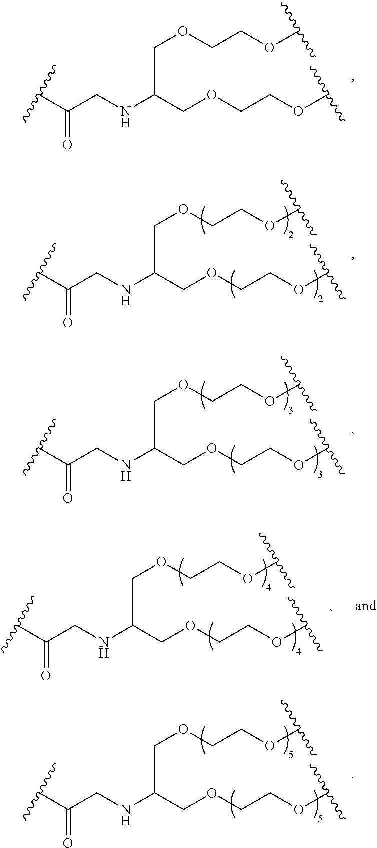

In certain embodiments Linker^{C} is selected from:

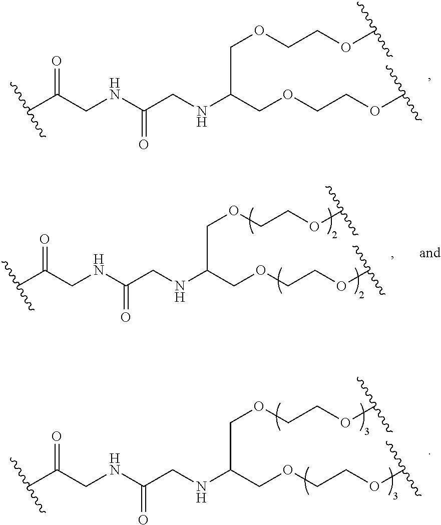

In certain embodiments Linker^{C} is selected from:

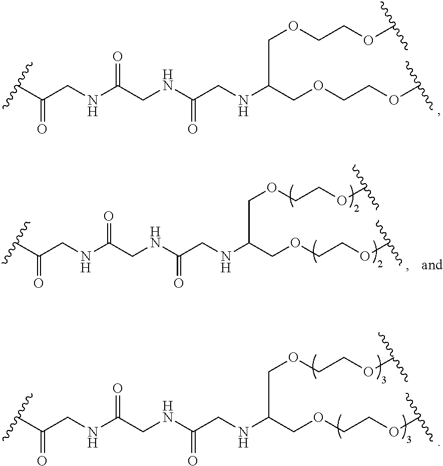

In certain embodiments Linker^{C} is selected from:

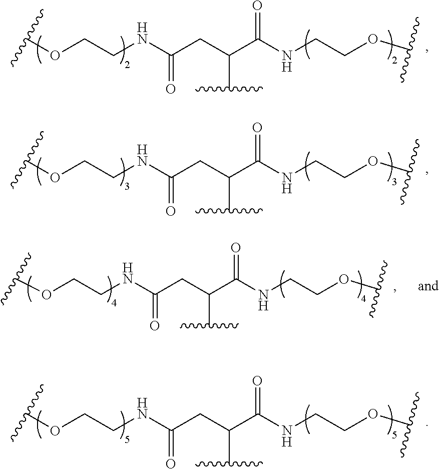

In certain embodiments Linker^{C} is selected from:

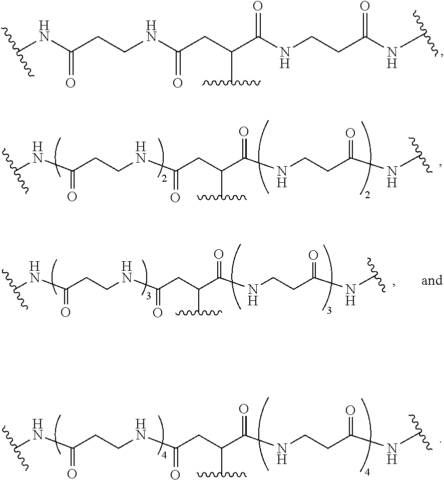

In certain embodiments Linker^{C} is selected from:

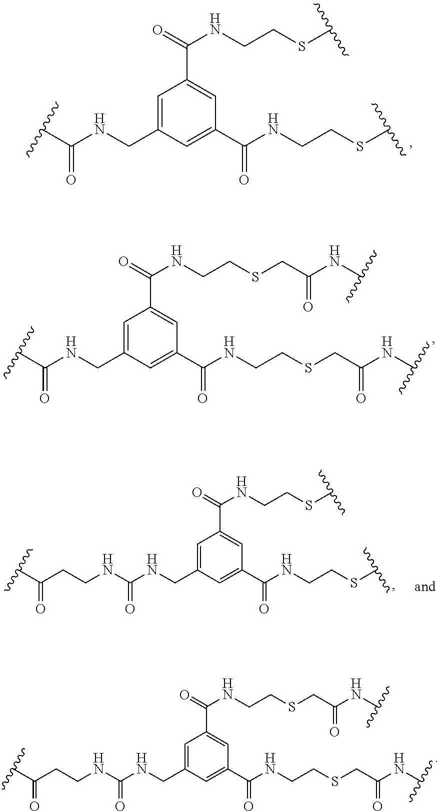 [SMILES: *SCCNC(=O)c1cc(CNC(*)=O)cc(C(=O)NCCS*)c1 ; *NC(=O)CSCCNC(=O)c1cc(CNC(*)=O)cc(C(=O)NCCSCC(=O)N*)c1 ; *SCCNC(=O)c1cc(CNC(=O)NCCC(*)=O)cc(C(=O)NCCS*)c1 ; *NC(=O)CSCCNC(=O)c1cc(CNC(=O)NCCC(*)=O)cc(C(=O)NCCSCC(=O)N*)c1]

In certain embodiments Linker^{C}-(Linker^{A})₂ is selected from:

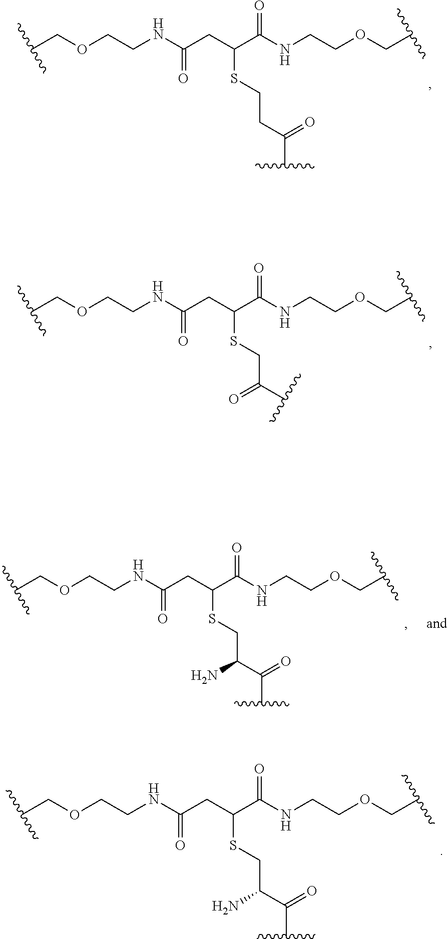 [SMILES: *COCCNC(=O)CC(SCCC(*)=O)C(=O)NCCOC* ; *COCCNC(=O)CC(SCC(*)=O)C(=O)NCCOC* ; *COCCNC(=O)CC(SC[C@H](N)C(*)=O)C(=O)NCCOC* ; *COCCNC(=O)CC(SC[C@@H](N)C(*)=O)C(=O)NCCOC*]

In certain embodiments Linker^{C}-(Linker^{A})₂ is selected from:

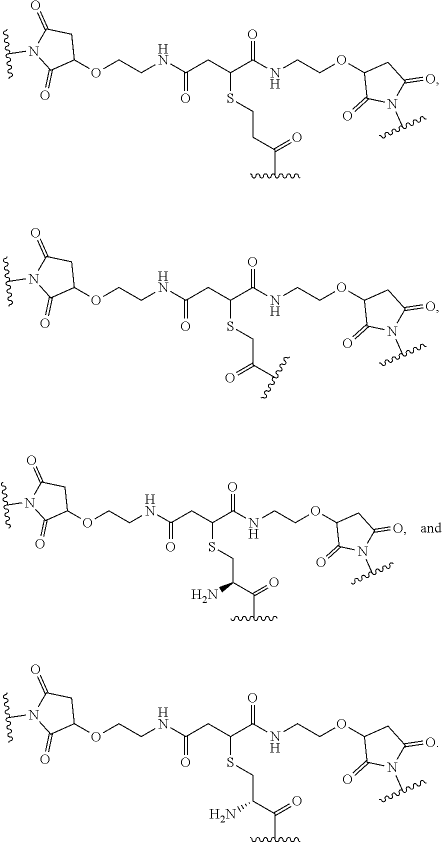 [SMILES: *C(=O)CCSC(CC(=O)NCCOC1CC(=O)N(*)C1=O)C(=O)NCCOC1CC(=O)N(*)C1=O ; *C(=O)CSC(CC(=O)NCCOC1CC(=O)N(*)C1=O)C(=O)NCCOC1CC(=O)N(*)C1=O ; *C(=O)[C@@H](N)CSC(CC(=O)NCCOC1CC(=O)N(*)C1=O)C(=O)NCCOC1CC(=O)N(*)C1=O ; *C(=O)[C@H](N)CSC(CC(=O)NCCOC1CC(=O)N(*)C1=O)C(=O)NCCOC1CC(=O)N(*)C1=O]

In certain embodiments Linker^{C}-(Linker^{A})₂ is selected from:

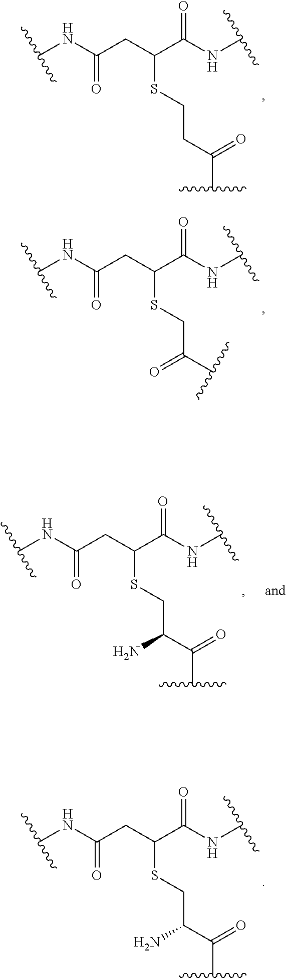

In certain embodiments Linker^{C}-(Linker^{A})₂ is selected from:

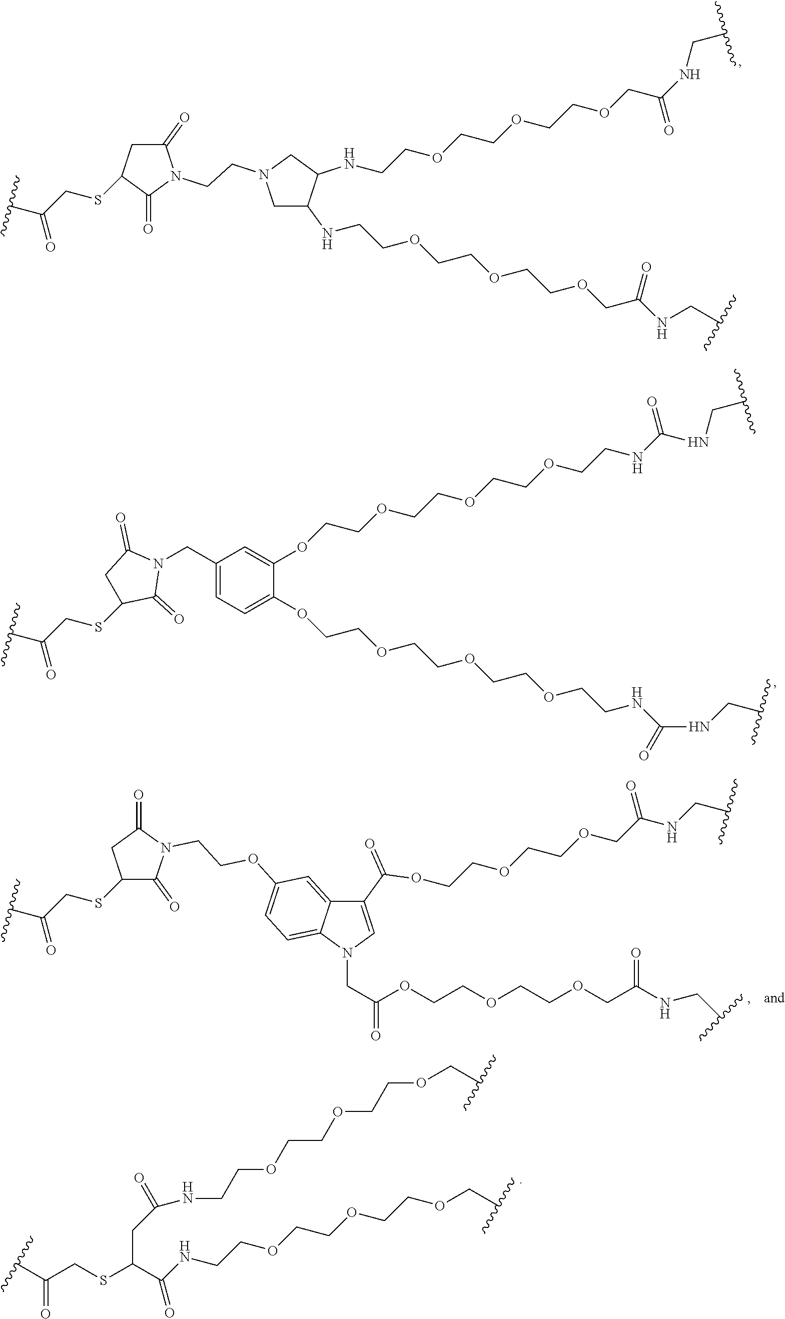

In certain embodiments, the Linker^{D} is selected from

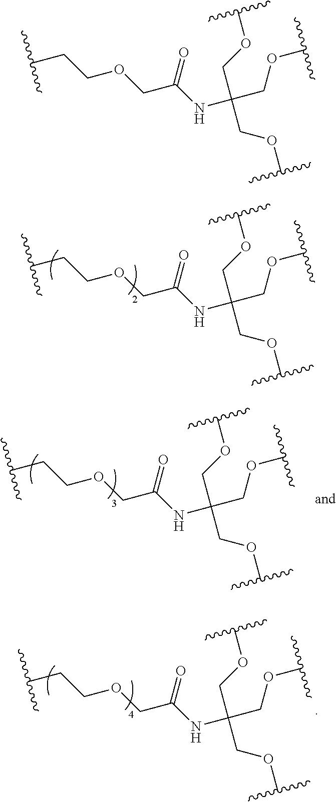

In certain embodiments, the Linker^{D} is selected from

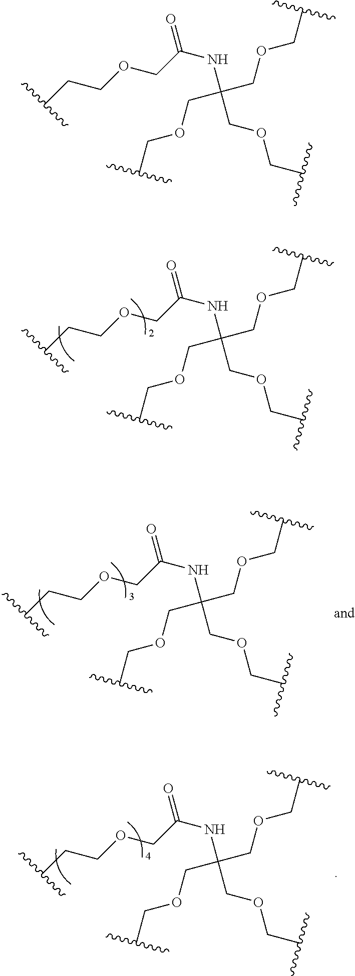

In certain embodiments, the Linker^{D} is selected from

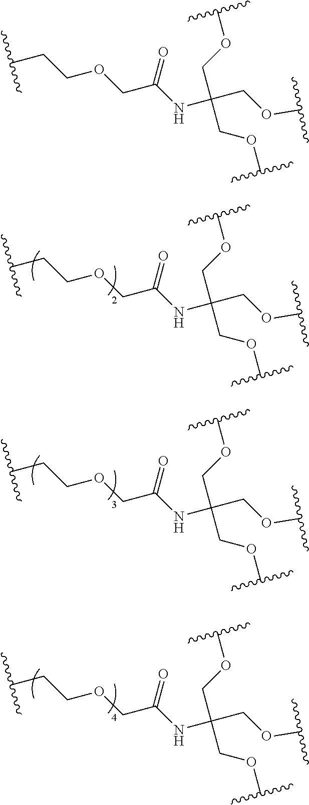

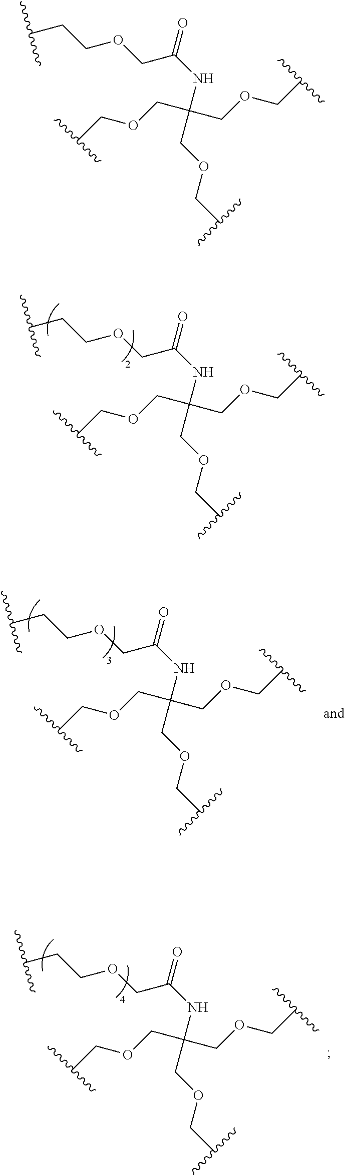

wherein each is optionally substituted with 1, 2, 3, or 4 substituents are selected from R²¹.

In certain embodiments, Linker^{B}-(Linker^{A}) is selected from

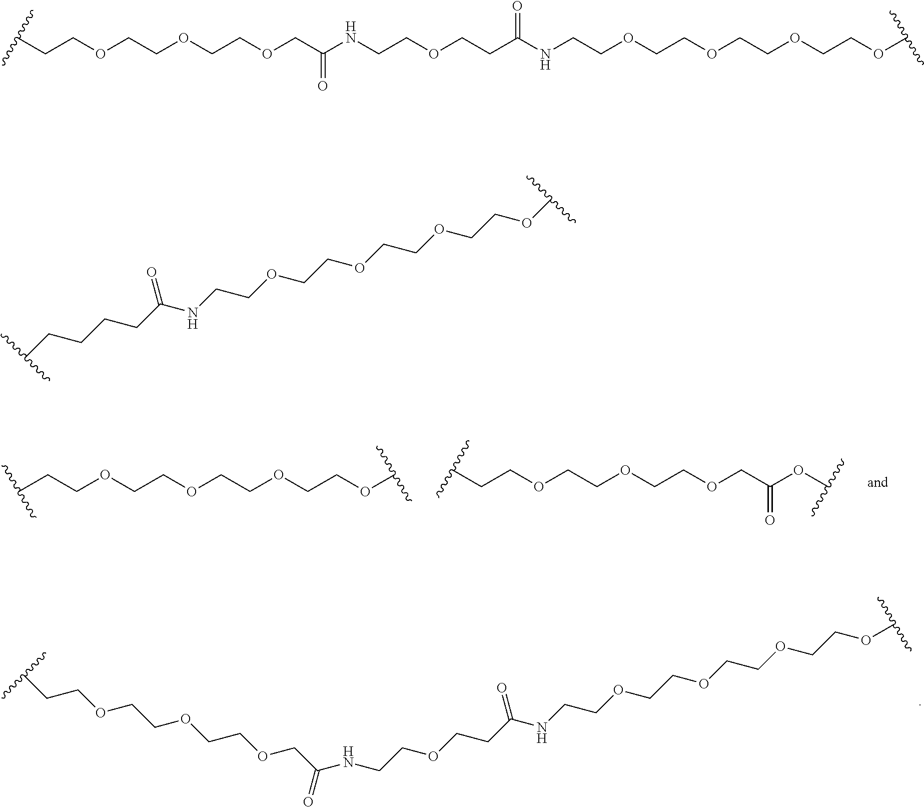 [SMILES: *CCOCCOCCOCC(=O)NCCOCCC(=O)NCCOCCOCCOCCO* ; *CCCCC(=O)NCCOCCOCCOCCO* ; *CCOCCOCCOCCO* ; *CCOCCOCCOCC(=O)O* ; *CCOCCOCCOCC(=O)NCCOCCC(=O)NCCOCCOCCOCCO*]

In certain embodiments, Linker^{C}-(Linker^{A}) is selected from

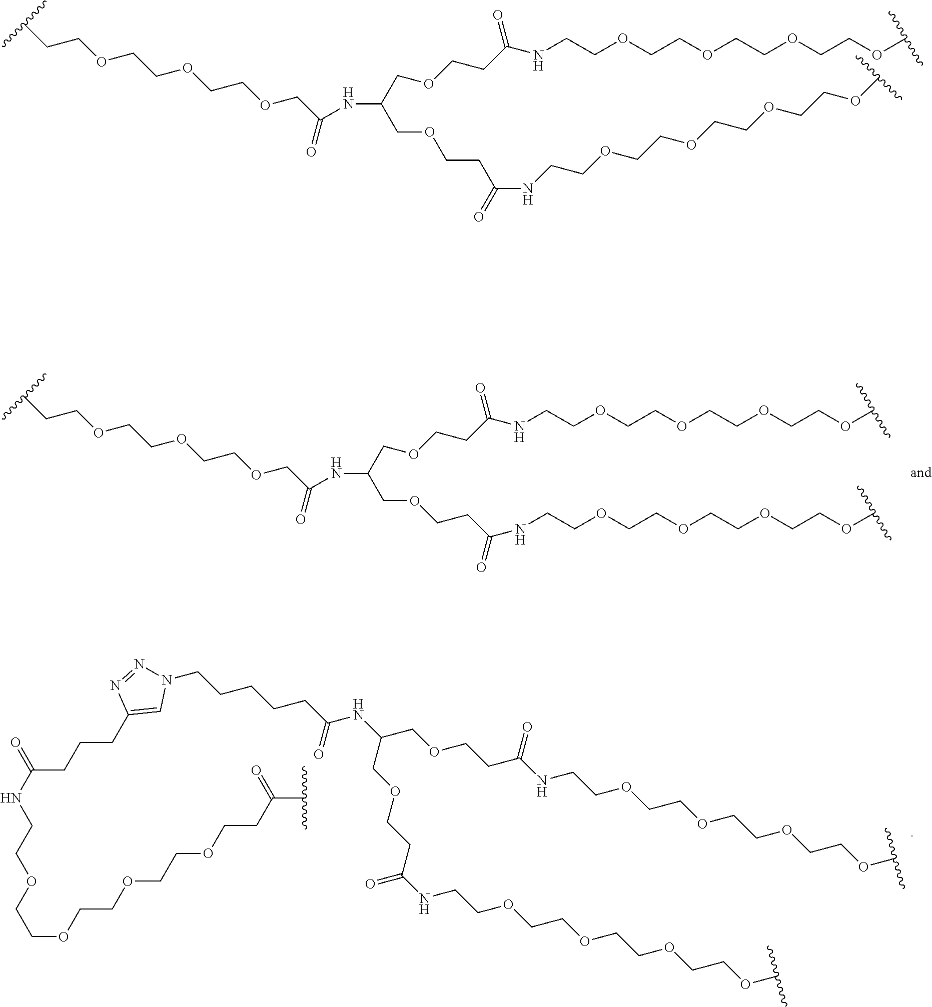 [SMILES: *CCOCCOCCOCC(=O)NC(COCCC(=O)NCCOCCOCCOCCO*)COCCC(=O)NCCOCCOCCOCCO* ; *CCOCCOCCOCC(=O)NC(COCCC(=O)NCCOCCOCCOCCO*)COCCC(=O)NCCOCCOCCOCCO* ; *OCCOCCOCCOCCNC(=O)CCOCC(COCCC(=O)NCCOCCOCCOCCO*)NC(=O)CCCCCn1cc(CCCC(=O)NCCOCCOCCOCCOCCC(*)=O)nn1]

In certain embodiments, Linker^{D}-(Linker^{A}) is selected from

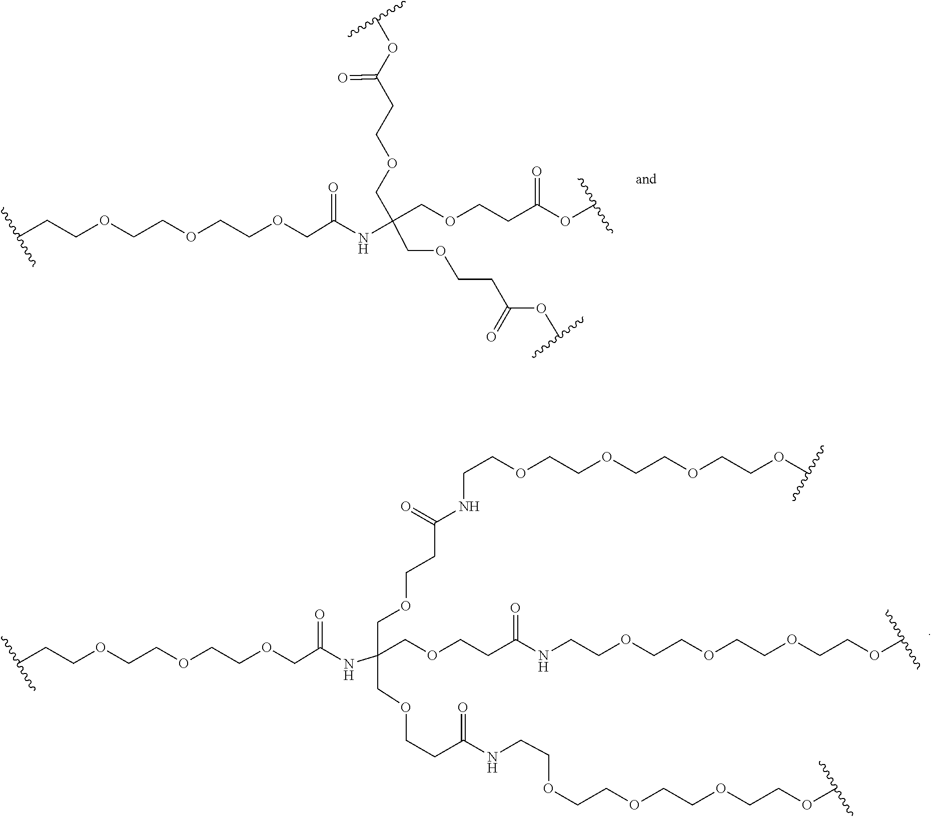 [SMILES: *CCOCCOCCOCC(=O)NC(COCCC(=O)O*)(COCCC(=O)O*)COCCC(=O)O* ; *CCOCCOCCOCC(=O)NC(COCCC(=O)NCCOCCOCCOCCO*)(COCCC(=O)NCCOCCOCCOCCO*)COCCC(=O)NCCOCCOCCOCCO*]

In certain embodiments Linker^{B} is selected from:

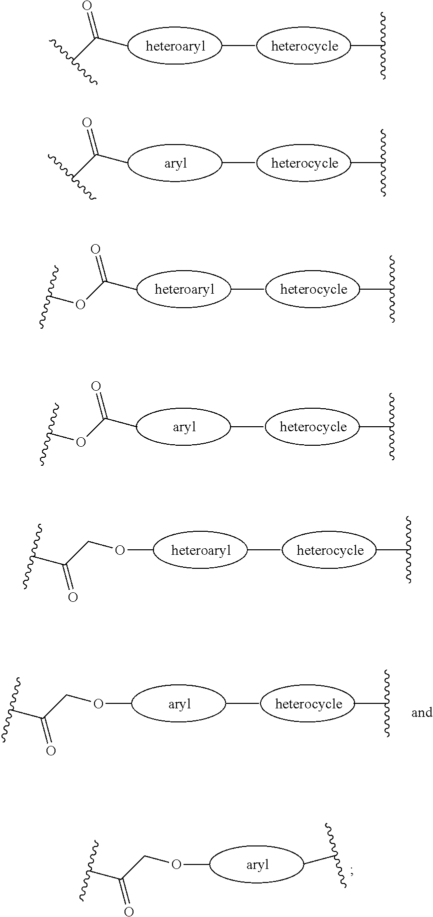

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

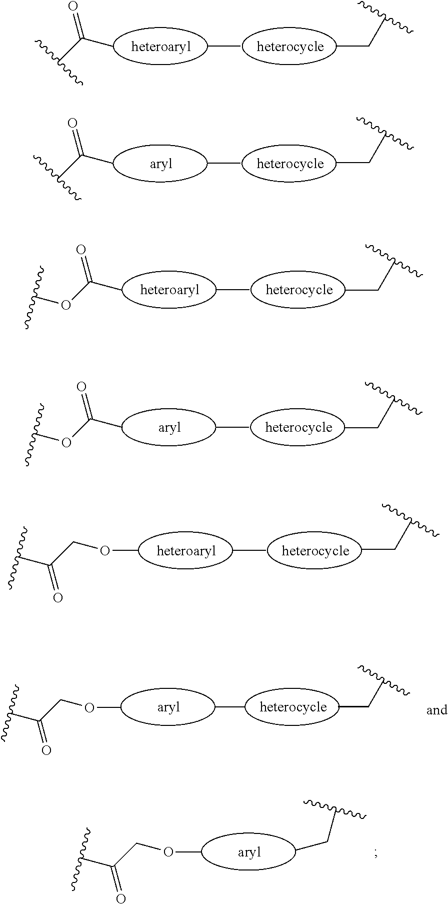

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B} is selected from:

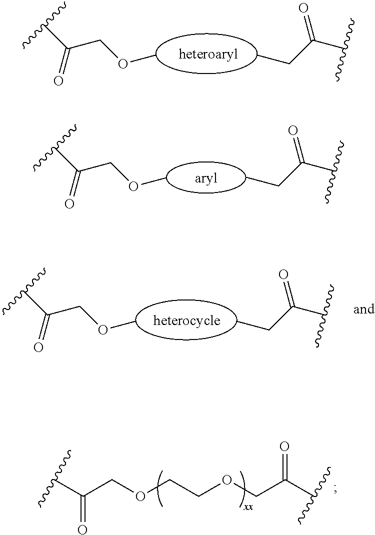

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{B}, Linker^{C}, or Linker^{D} is selected from:

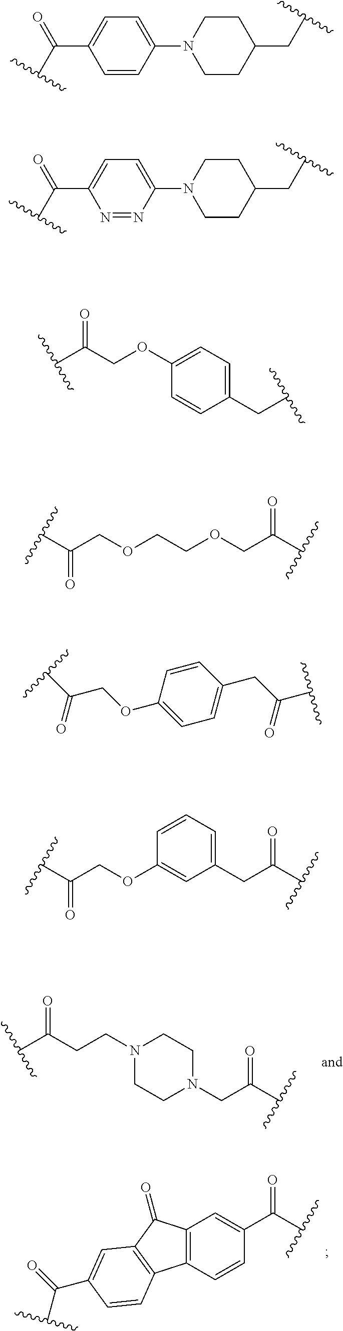

wherein each heteroaryl, heterocycle, cycloalkyl, and aryl can optionally be substituted with 1, 2, 3, or 4 of any combination of halogen, alkyl, haloalkyl, aryl, heteroaryl, heterocycle, or cycloalkyl, as allowed by valence.

In certain embodiments Linker^{A} is selected from:

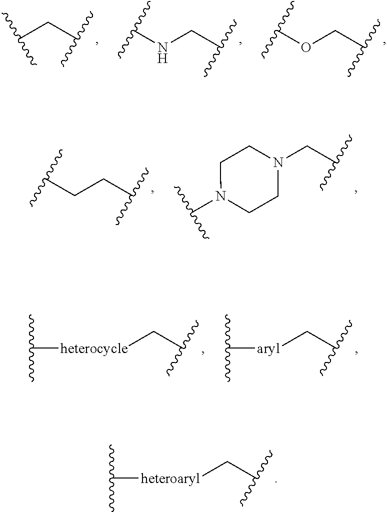

In certain embodiments Linker^{A} is selected from:

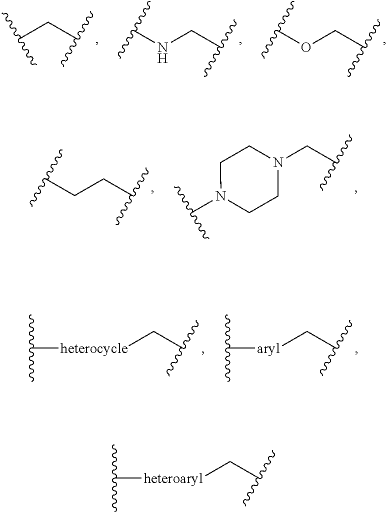

each of which is substituted with 1 or 2 optional substituents.

In certain embodiments Linker^{A} is bond.

In certain embodiments the left side of Linker^{A} is attached to the ASGPR Binding Ligand and the right side is attached to Linker^{B}, Linker^{C}, or Linker^{D}.

In certain embodiments the right side of Linker^{A} is attached to the ASGPR Binding Ligand and the right side is attached to Linker^{B}, Linker^{C}, or Linker^{D}.

In certain embodiments Linker^{B} is selected from:

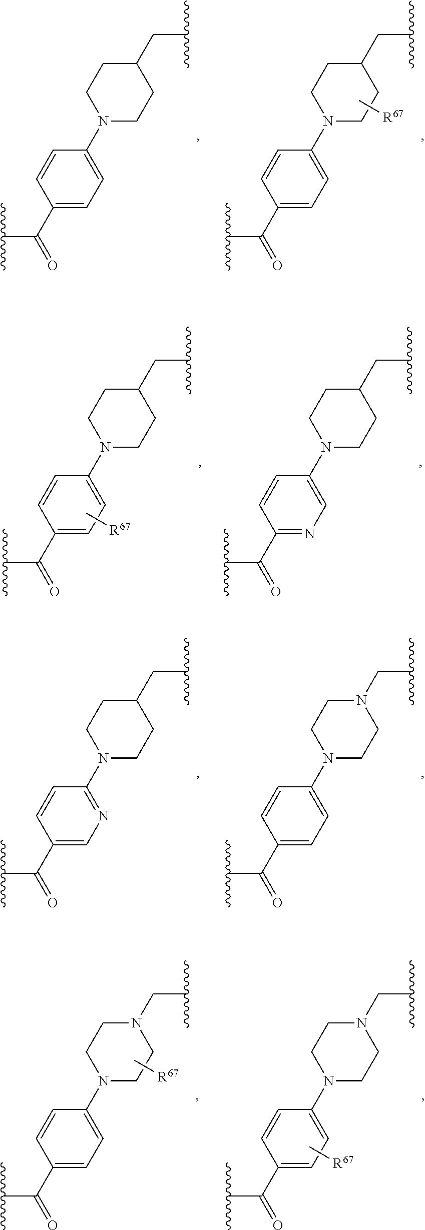

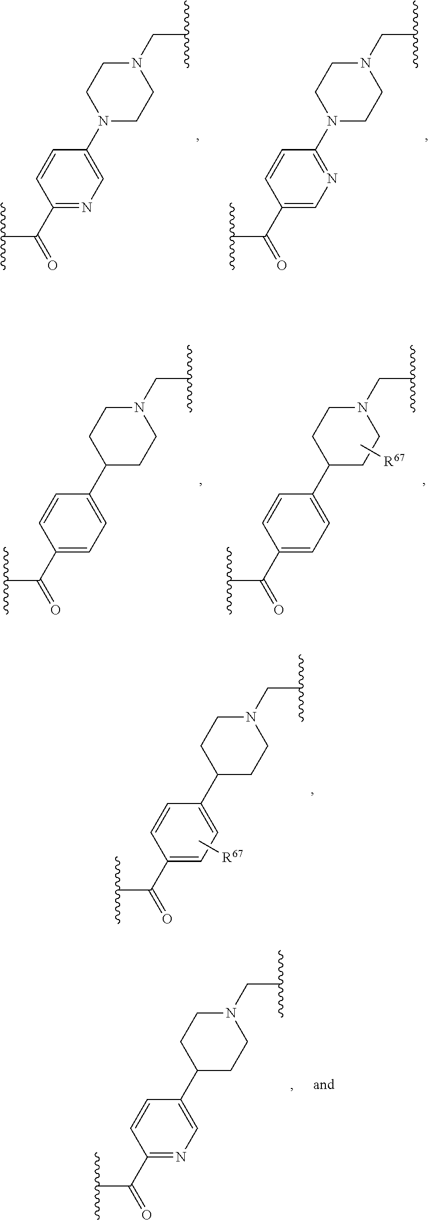

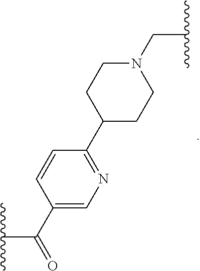 [SMILES: *CN1CCC(c2ccc(C(*)=O)cn2)CC1]

In certain embodiments Linker^{B} is selected from:

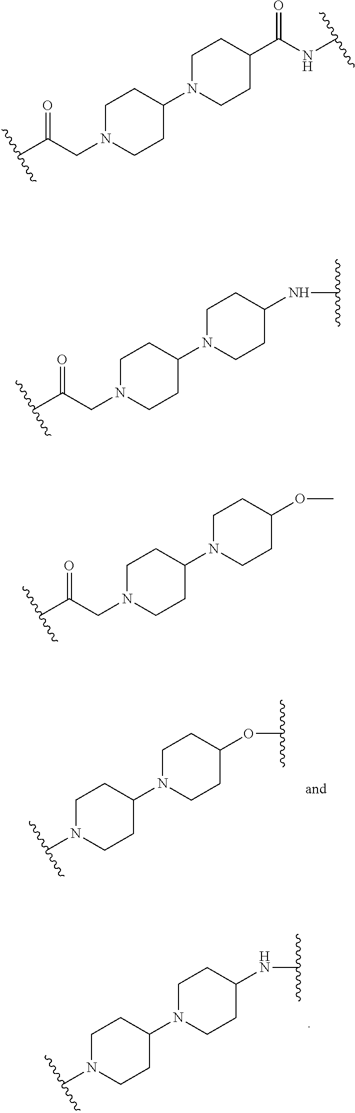 [SMILES: *NC(=O)C1CCN(C2CCN(CC(*)=O)CC2)CC1 ; *NC1CCN(C2CCN(CC(*)=O)CC2)CC1 ; *C(=O)CN1CCC(N2CCC(OC)CC2)CC1 ; *OC1CCN(C2CCN(*)CC2)CC1 ; *NC1CCN(C2CCN(*)CC2)CC1]

In certain embodiments Linker^{B} is selected from:

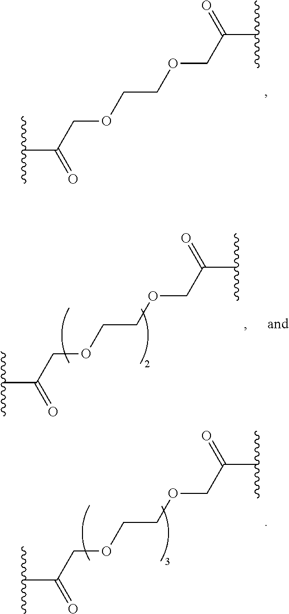

In certain embodiments the left side of Linker^{B} is attached to the Extracellular Targeting Ligand and the right side is attached to Linker^{A}

In certain embodiments the right side of Linker^{B} is attached to the Extracellular Targeting Ligand and the left side is attached to Linker^{A}.

In certain embodiments Linker^{B} is bond.

In alternative embodiments a linker is provided as described above wherein a

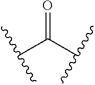 [SMILES: *C(*)=O]

is replaced with a

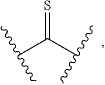 [SMILES: *C(*)=S]

for example where Linker^{B} is drawn as

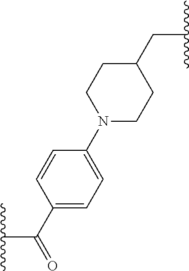 [SMILES: *CC1CCN(c2ccc(C(*)=O)cc2)CC1]

it is s

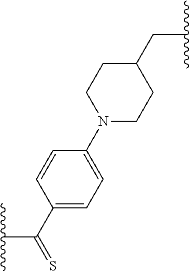 [SMILES: *CC1CCN(c2ccc(C(*)=S)cc2)CC1]

in this embodiment.

In alternative embodiments a linker is provided as described above wherein a

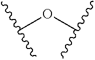 [SMILES: *O*]

is replaced with a

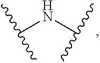 [SMILES: *N*]

for example where Linker^{B} is drawn as

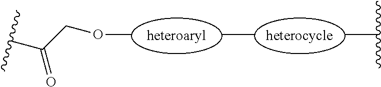

it

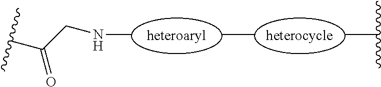

in this embodiment.

In alternative embodiments a linker is provided as described above wherein a

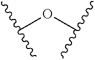 [SMILES: *O*]

is replaced with a

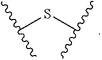 [SMILES: *S*]

In certain embodiments the ASGPR Binding Ligand is selected from:

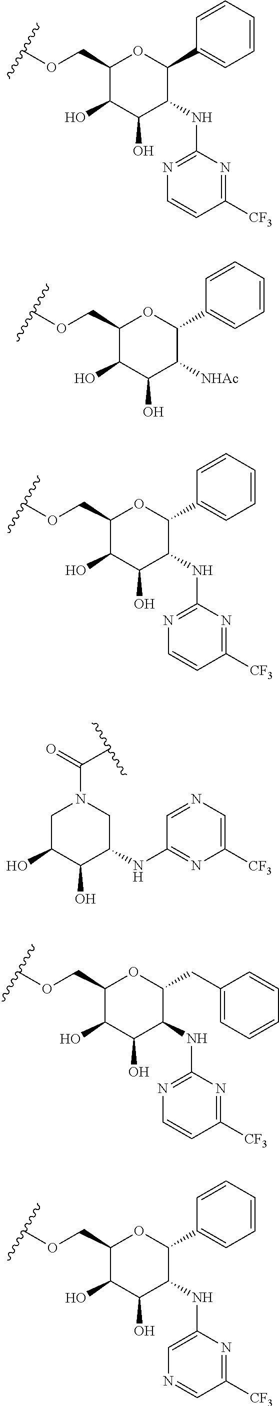

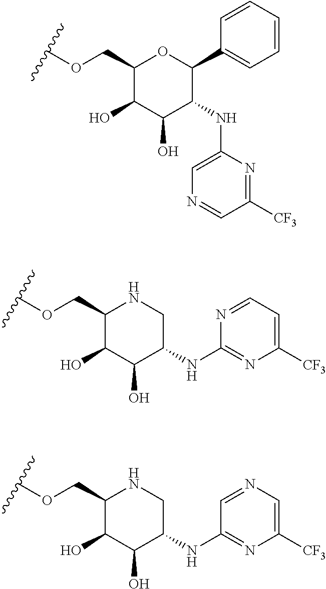 [SMILES: *OC[C@H]1O[C@@H](c2ccccc2)[C@H](Nc2cncc(C(F)(F)F)n2)[C@@H](O)[C@H]1O ; *OC[C@H]1NC[C@H](Nc2nccc(C(F)(F)F)n2)[C@@H](O)[C@H]1O ; *OC[C@H]1NC[C@H](Nc2cncc(C(F)(F)F)n2)[C@@H](O)[C@H]1O]

### Embodiments of A

In certain embodiments A is a 6-membered heteroaryl.

In certain embodiments A is a 5-membered heteroaryl.

In certain embodiments A is a bicyclic heteroaryl.

In certain embodiments

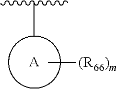

is selected from:

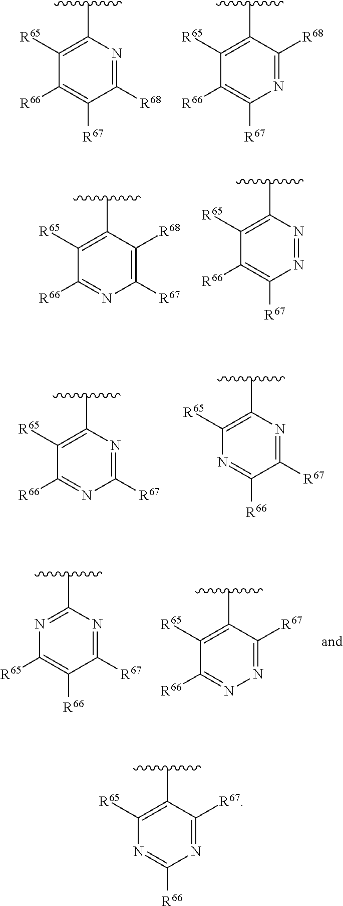

In certain embodiments

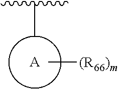

is selected from:

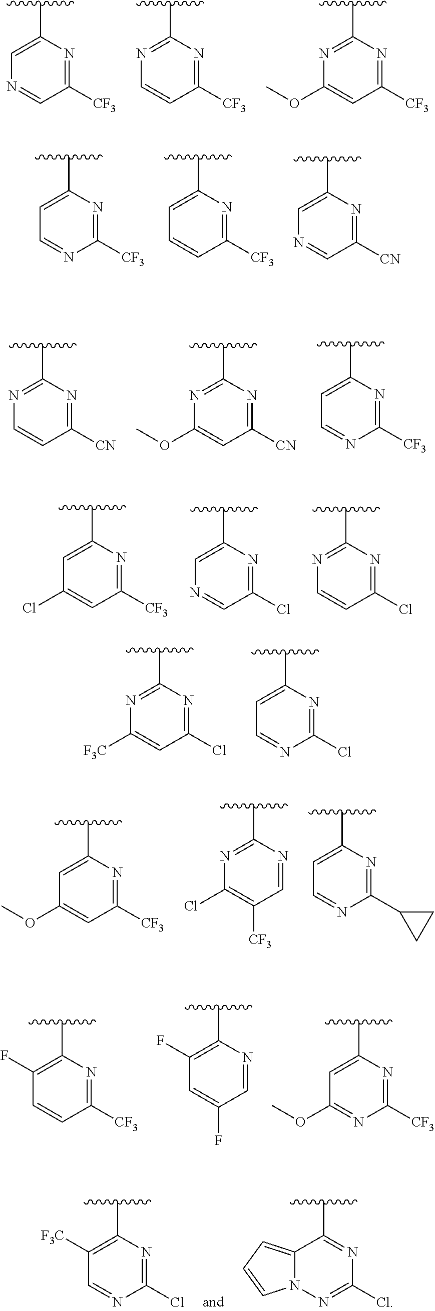

In certain embodiments

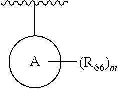

is

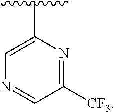 [SMILES: *c1cncc(C(F)(F)F)n1]

In certain embodiments

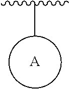

is

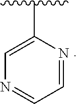 [SMILES: *c1cnccn1]

In certain embodiments m is 0.

In certain embodiments m is 1.

In certain embodiments m is 2.

In certain embodiments m is 3.

### Embodiments of B

In certain embodiments B is aryl.

In certain embodiments B is heterocycle.

In certain embodiments B is cycloalkyl.

In certain embodiments B is heteroaryl.

In certain embodiments B is a 6-membered heteroaryl.

In certain embodiments B is a 5-membered heteroaryl.

In certain embodiments B is a bicyclic heteroaryl.

In certain embodiments

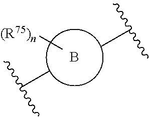

is selected from

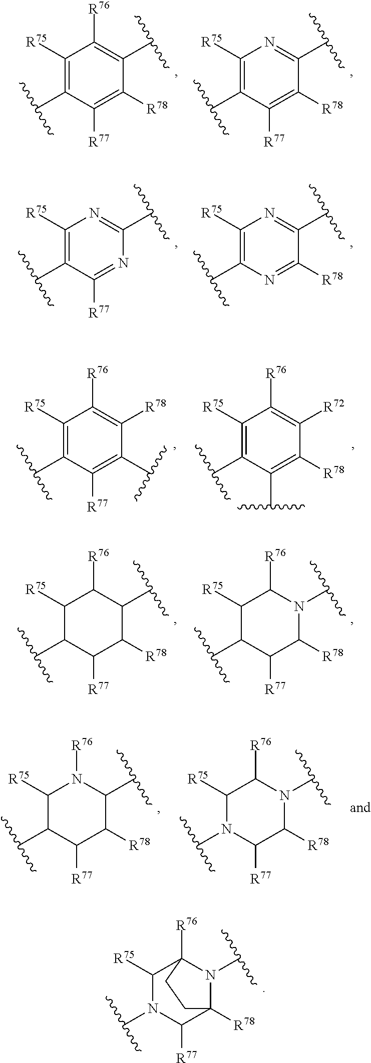

In certain embodiments

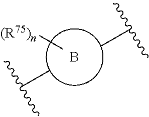

is selected from

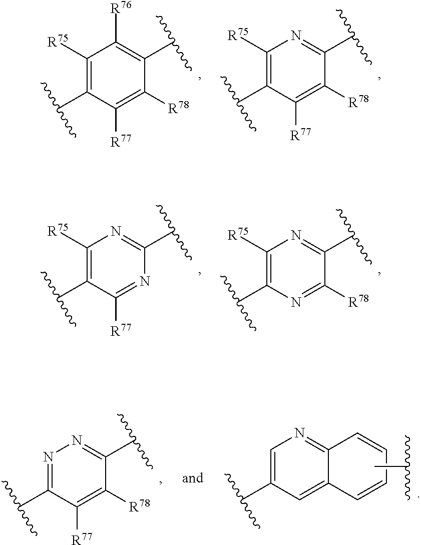

In certain embodiments

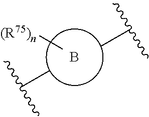

is

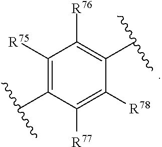

In certain embodiments

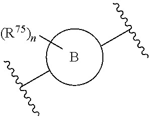

is

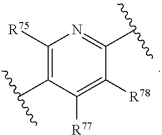

In certain embodiments

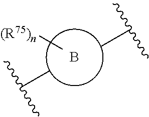

is

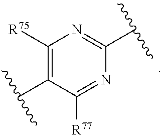

In certain embodiments

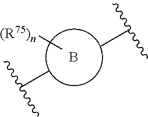

is

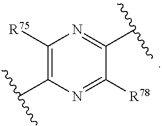

In certain embodiments

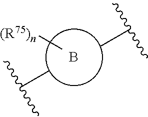

is

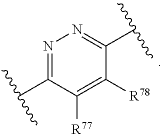

In certain embodiments

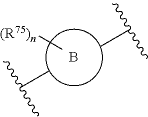

is

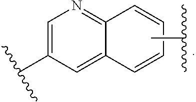

In certain embodiments

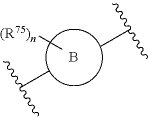

is selected from

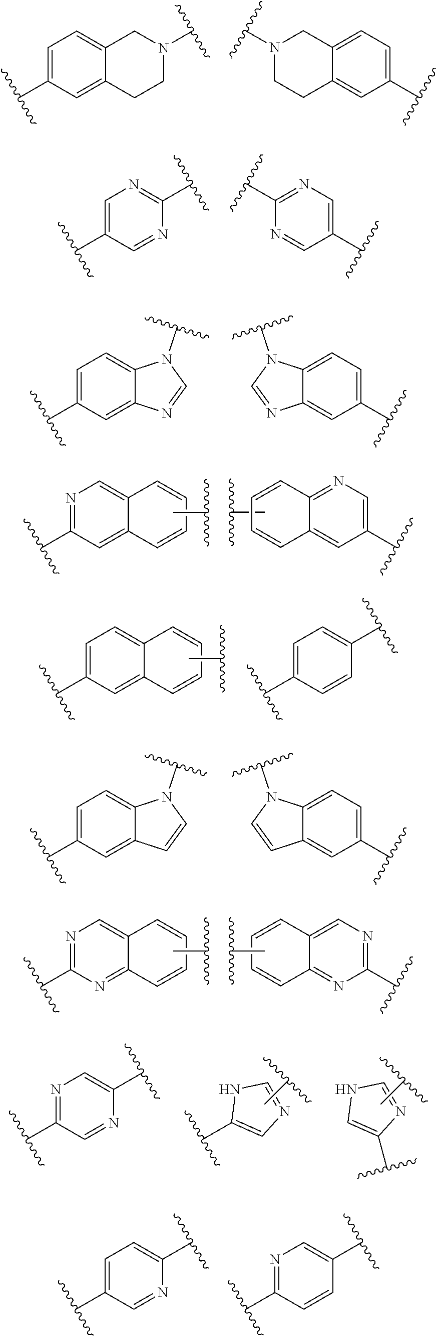

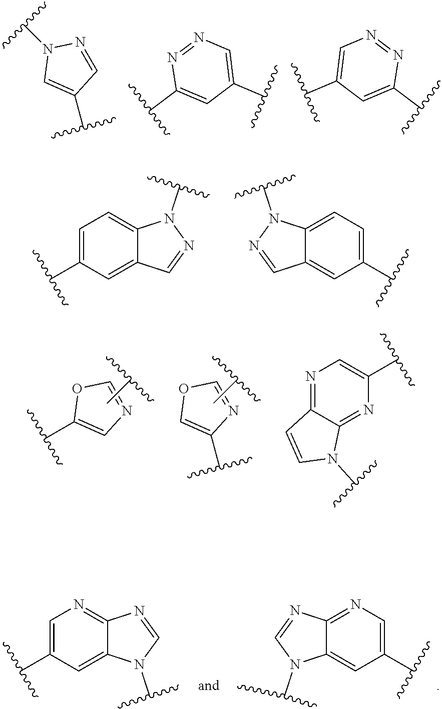

In certain embodiments

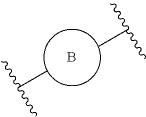

is selected from

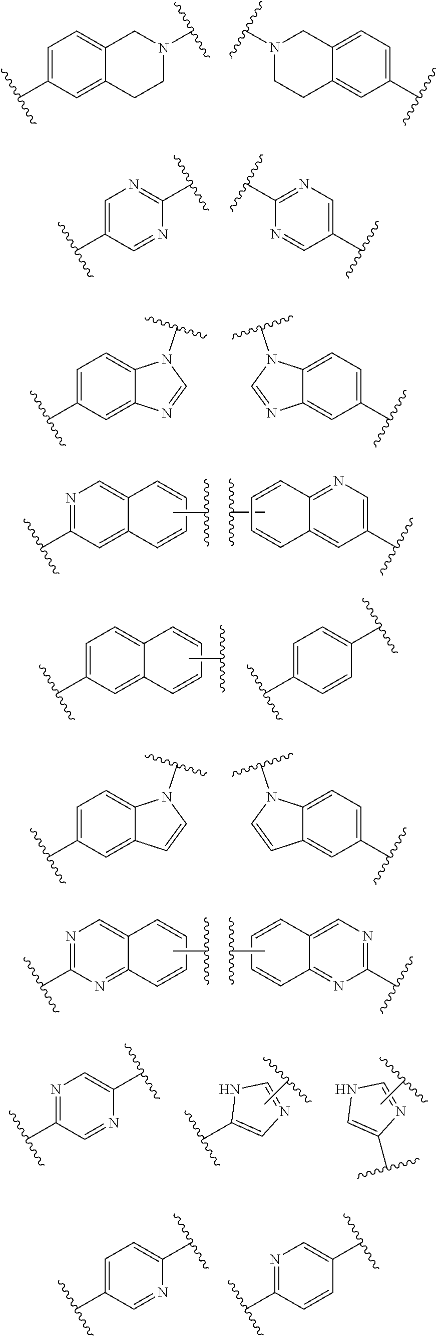

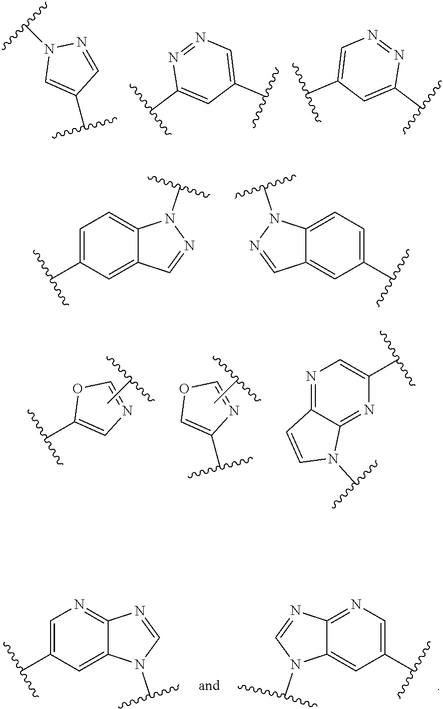

In certain embodiments n is 0.

In certain embodiments n is 1.

In certain embodiments n is 2.

In certain embodiments n is 3.

### Embodiments of C

In certain embodiments C is aryl.

In certain embodiments C is heterocycle.

In certain embodiments C is cycloalkyl.

In certain embodiments C is heteroaryl.

In certain embodiments

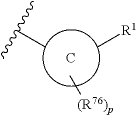

is selected from:

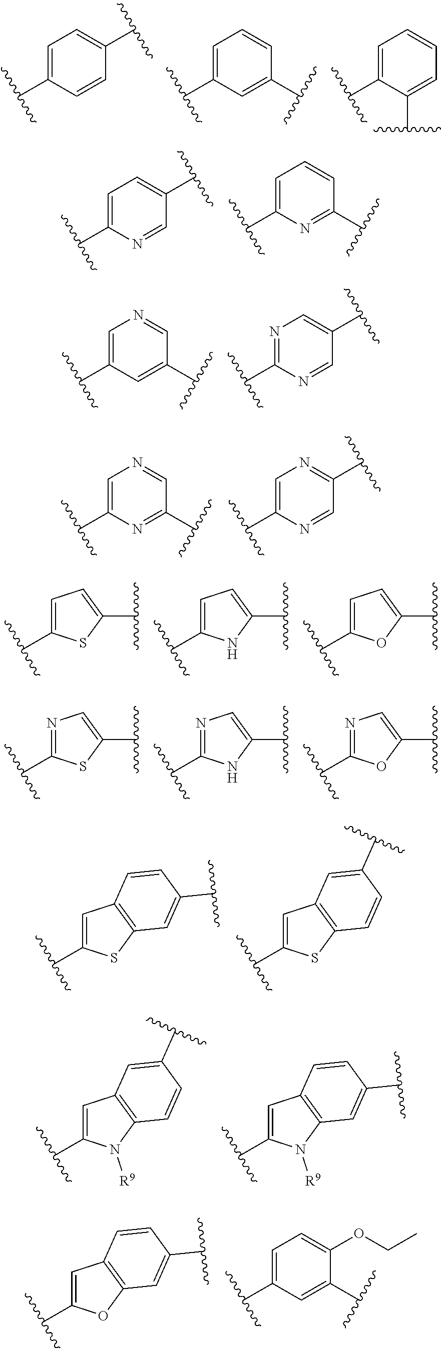

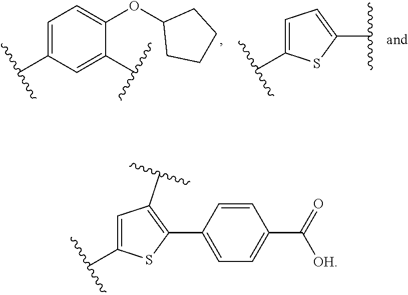 [SMILES: *c1ccc(OC2CCCC2)c(*)c1 ; *c1ccc(*)s1 ; *c1cc(*)c(-c2ccc(C(=O)O)cc2)s1]

In certain embodiments

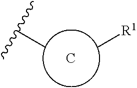

is selected from:

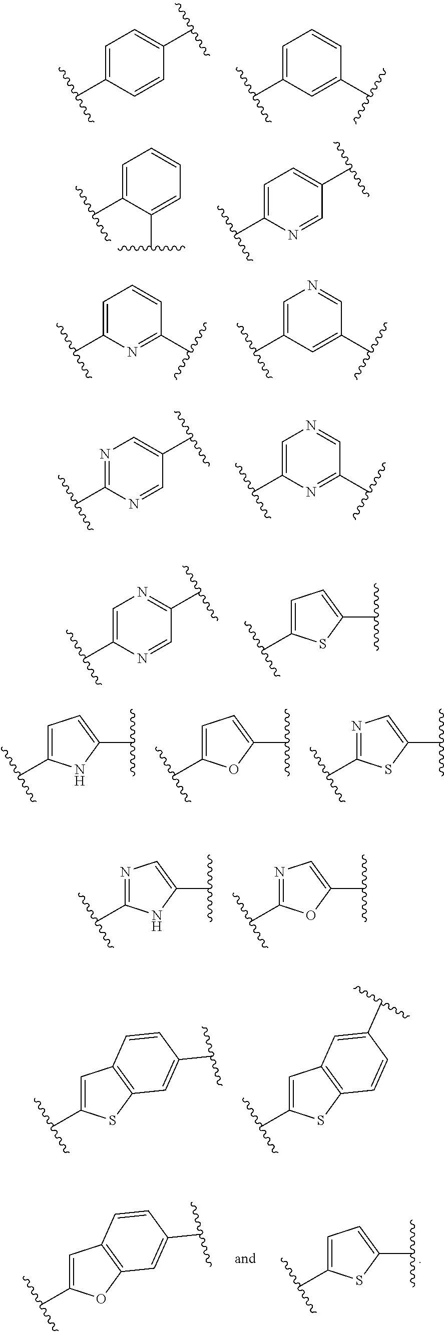

Wherein for embodiments of A, B, and C the configuration drawn left to right and the configuration drawn right to left are both contemplated by the embodiments described herein.

For example, if the ASGPR Binding Ligand is a compound of Formula

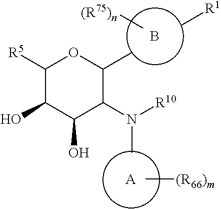

### and B is

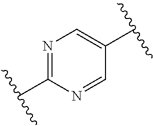 [SMILES: *c1cnc(*)nc1]

then both

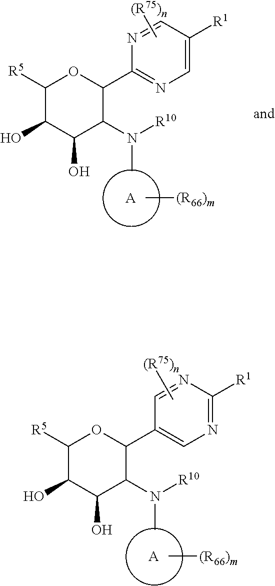

are described and when

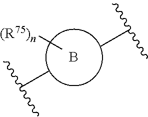

is

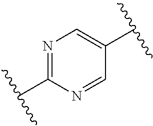 [SMILES: *c1cnc(*)nc1]

then both

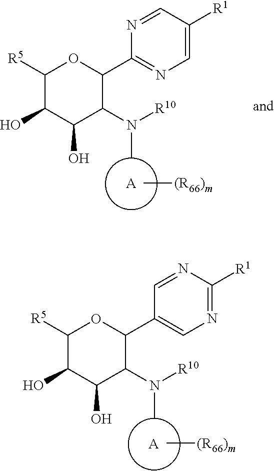

are described.

In certain embodiments p is 0.

In certain embodiments p is 1.

In certain embodiments p is 2.

In certain embodiments p is 3.

### Embodiments of R¹

In certain embodiments R¹ is hydrogen.

In certain embodiments R¹ is

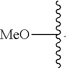 [SMILES: *OC]

In certain embodiments R¹ is

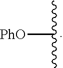 [SMILES: *Oc1ccccc1]

In certain embodiments R¹ is

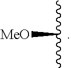

In certain embodiments R¹ is

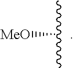

In certain embodiments R¹ is

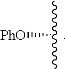

In certain embodiments R¹ is

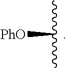

In certain embodiments R¹ is C₀-C₆alkyl-cyano optionally substituted with 1, 2, 3, or 4 substituents.

In certain embodiments R¹ is alkyl optionally substituted with 1, 2, 3, or 4 substituents.

In certain embodiments R¹ is alkenyl optionally substituted with 1, 2, 3, or 4 substituents.

In certain embodiments R¹ is alkynyl optionally substituted with 1, 2, 3, or 4 substituents.

In certain embodiments R¹ is haloalkyl optionally substituted with 1, 2, 3, or 4 substituents.

In certain embodiments R¹ is F.

In certain embodiments R¹ is Cl.

In certain embodiments R¹ is Br.

In certain embodiments R¹ is aryl optionally substituted with 1, 2, 3, or 4 substituents.

In certain embodiments R¹ is arylalkyl optionally substituted with 1, 2, 3, or 4 substituents.

In certain embodiments R¹ is heteroaryl optionally substituted with 1, 2, 3, or 4 substituents.

In certain embodiments R¹ is heteroarylalkyl optionally substituted with 1, 2, 3, or 4 substituents.

In certain embodiments R¹ is heterocycle optionally substituted with 1, 2, 3, or 4 substituents.

In certain embodiments R¹ is heterocycloalkyl optionally substituted with 1, 2, 3, or 4 substituents.

In certain embodiments R¹ is haloalkoxy optionally substituted with 1, 2, 3, or 4 substituents.

In certain embodiments R¹ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

### Embodiments of R^{11B}

In certain embodiments R^{11B} is hydrogen.

In certain embodiments R^{11B} is

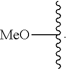 [SMILES: *OC]

In certain embodiments R^{11B} is

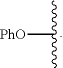 [SMILES: *Oc1ccccc1]

In certain embodiments R^{11B} is

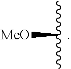

In certain embodiments R^{11B} is

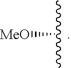

In certain embodiments R^{11B} is

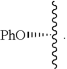

In certain embodiments R^{11B} is

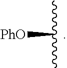

In certain embodiments R^{11B} is C₀-C₆alkyl-cyano optionally substituted with 1, 2, 3, or 4 substituents.

In certain embodiments R^{11B} is alkyl optionally substituted with 1, 2, 3, or 4 substituents.

In certain embodiments R^{11B} is alkenyl optionally substituted with 1, 2, 3, or 4 substituents.

In certain embodiments R^{11B} is alkynyl optionally substituted with 1, 2, 3, or 4 substituents.

In certain embodiments R^{11B} is haloalkyl optionally substituted with 1, 2, 3, or 4 substituents.

In certain embodiments R^{11B} is F.

In certain embodiments R^{11B} is Cl.

In certain embodiments R^{11B} is Br.

In certain embodiments R^{11B} is aryl optionally substituted with 1, 2, 3, or 4 substituents.

In certain embodiments R^{11B} is arylalkyl optionally substituted with 1, 2, 3, or 4 substituents.

In certain embodiments R^{11B} is heteroaryl optionally substituted with 1, 2, 3, or 4 substituents.

In certain embodiments R^{11B} is heteroarylalkyl optionally substituted with 1, 2, 3, or 4 substituents.

In certain embodiments R^{11B} is heterocycle optionally substituted with 1, 2, 3, or 4 substituents.

In certain embodiments R^{11B} is heterocycloalkyl optionally substituted with 1, 2, 3, or 4 substituents.

In certain embodiments R^{11B} is haloalkoxy optionally substituted with 1, 2, 3, or 4 substituents.

In certain embodiments R^{11B} is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R′)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

### Embodiments of R⁵

In certain embodiments R⁵ is hydrogen.

In certain embodiments R⁵ is

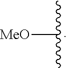 [SMILES: *OC]

In certain embodiments R⁵ is

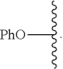 [SMILES: *Oc1ccccc1]

In certain embodiments R⁵ is

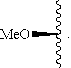

In certain embodiments R⁵ is

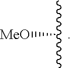

In certain embodiments R⁵ is

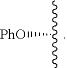

In certain embodiments R⁵ is

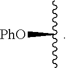

In certain embodiments R⁵ is C₀-C₆alkyl-cyano optionally substituted with 1, 2, 3, or 4 substituents.

In certain embodiments R⁵ is alkyl optionally substituted with 1, 2, 3, or 4 substituents.

In certain embodiments R⁵ is alkenyl optionally substituted with 1, 2, 3, or 4 substituents.

In certain embodiments R⁵ is alkynyl optionally substituted with 1, 2, 3, or 4 substituents.

In certain embodiments R⁵ is haloalkyl optionally substituted with 1, 2, 3, or 4 substituents.

In certain embodiments R⁵ is F.

In certain embodiments R⁵ is Cl.

In certain embodiments R⁵ is Br.

In certain embodiments R⁵ is aryl optionally substituted with 1, 2, 3, or 4 substituents.

In certain embodiments R⁵ is arylalkyl optionally substituted with 1, 2, 3, or 4 substituents.

In certain embodiments R⁵ is heteroaryl optionally substituted with 1, 2, 3, or 4 substituents.

In certain embodiments R⁵ is heteroarylalkyl optionally substituted with 1, 2, 3, or 4 substituents.

In certain embodiments R⁵ is heterocycle optionally substituted with 1, 2, 3, or 4 substituents.

In certain embodiments R⁵ is heterocycloalkyl optionally substituted with 1, 2, 3, or 4 substituents.

In certain embodiments R⁵ is haloalkoxy optionally substituted with 1, 2, 3, or 4 substituents.

In certain embodiments R⁵ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R⁸)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

### Embodiments of R⁵⁵

In certain embodiments R⁵⁵ is hydrogen.

In certain embodiments R⁵⁵ is

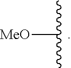 [SMILES: *OC]

In certain embodiments R⁵⁵ is

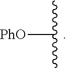 [SMILES: *Oc1ccccc1]

In certain embodiments R⁵⁵ is

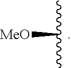

In certain embodiments R⁵⁵ is

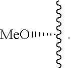

In certain embodiments R⁵⁵ is

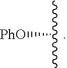

In certain embodiments R⁵⁵ is

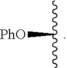

In certain embodiments R⁵⁵ is C₀-C₆alkyl-cyano optionally substituted with 1, 2, 3, or 4 substituents.

In certain embodiments R⁵⁵ is alkyl optionally substituted with 1, 2, 3, or 4 substituents.

In certain embodiments R⁵⁵ is alkenyl optionally substituted with 1, 2, 3, or 4 substituents.

In certain embodiments R⁵⁵ is alkynyl optionally substituted with 1, 2, 3, or 4 substituents.

In certain embodiments R⁵⁵ is haloalkyl optionally substituted with 1, 2, 3, or 4 substituents.

In certain embodiments R⁵⁵ is F.

In certain embodiments R⁵⁵ is Cl.

In certain embodiments R⁵⁵ is Br.

In certain embodiments R⁵⁵ is aryl optionally substituted with 1, 2, 3, or 4 substituents.

In certain embodiments R⁵⁵ is arylalkyl optionally substituted with 1, 2, 3, or 4 substituents.

In certain embodiments R⁵⁵ is heteroaryl optionally substituted with 1, 2, 3, or 4 substituents.

In certain embodiments R⁵⁵ is heteroarylalkyl optionally substituted with 1, 2, 3, or 4 substituents.

In certain embodiments R⁵⁵ is heterocycle optionally substituted with 1, 2, 3, or 4 substituents.

In certain embodiments R⁵⁵ is heterocycloalkyl optionally substituted with 1, 2, 3, or 4 substituents.

In certain embodiments R⁵⁵ is haloalkoxy optionally substituted with 1, 2, 3, or 4 substituents.

In certain embodiments R⁵⁵ is —O-alkenyl, —O-alkynyl, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-N(R⁸)—C(O)R³, C₀-C₆alkyl-N(R⁸)—S(O)R³, C₀-C₆alkyl-N(R′)—C(S)R³, C₀-C₆alkyl-N(R⁸)—S(O)₂R³, C₀-C₆alkyl-O—C(O)R³, C₀-C₆alkyl-O—S(O)R³, C₀-C₆alkyl-O—C(S)R³, —N═S(O)(R³)₂, C₀-C₆alkylN₃, or C₀-C₆alkyl-O—S(O)₂R³, each of which is optionally substituted with 1, 2, 3, or 4 substituents.

### Embodiments of R¹⁰

In certain embodiments R¹⁰ is hydrogen.

In certain embodiments R¹⁰ is alkyl.

In certain embodiments R¹⁰ is haloalkyl.

In certain embodiments R¹⁰ is C(O)R³.

### Embodiments of ASGPR Binder

1. In certain embodiments a compound of Formula I, Formula II or Formula III or an ASGPR Binding Ligand of the present invention is provided wherein:
A is a heteroaryl; and
R⁶⁵, R⁶⁶, R⁶⁷, and R⁶⁸ are independently selected from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰.
2. The ASGPR binding ligand or extracellular protein degrading compound of embodiment 1, wherein

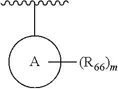

is

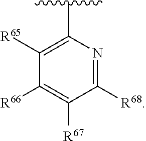

3. The ASGPR binding ligand or extracellular protein degrading compound of embodiment 1, wherein

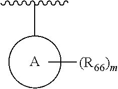

is

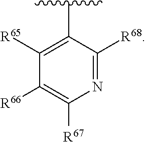

4. The ASGPR binding ligand or extracellular protein degrading compound of embodiment 1, wherein

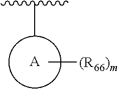

is

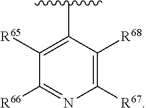

5. The ASGPR binding ligand or extracellular protein degrading compound of embodiment 1, wherein

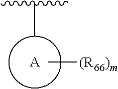

is

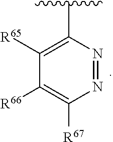

6. The ASGPR binding ligand or extracellular protein degrading compound of embodiment 1, wherein

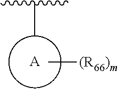

is

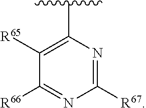

7. The ASGPR binding ligand or extracellular protein degrading compound of embodiment 1, wherein

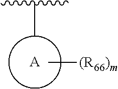

is

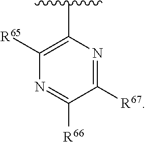

8. The ASGPR binding ligand or extracellular protein degrading compound of embodiment 1, wherein

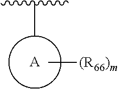

is

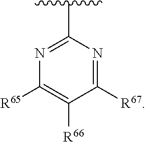

9. The ASGPR binding ligand or extracellular protein degrading compound of embodiment 1, wherein

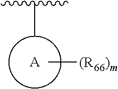

is

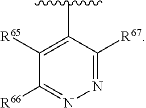

10. The ASGPR binding ligand or extracellular protein degrading compound of embodiment 1, wherein

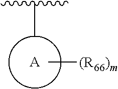

is

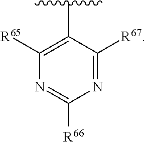

11. The ASGPR binding ligand or extracellular protein degrading compound of any one of embodiments 1-10, wherein R⁶⁵ is not substituted with an optional substituent.
12. The ASGPR binding ligand or extracellular protein degrading compound of any one of embodiments 1-10, wherein R⁶⁵ is substituted with 1 optional substituent.
13. The ASGPR binding ligand or extracellular protein degrading compound of any one of embodiments 1-10, wherein R⁶⁵ is substituted with 2 optional substituents.
14. The ASGPR binding ligand or extracellular protein degrading compound of any one of embodiments 1-10, wherein R⁶⁵ is substituted with 3 optional substituents.
15. The ASGPR binding ligand or extracellular protein degrading compound of any one of embodiments 1-10, wherein R⁶⁵ is hydrogen.
16. The ASGPR binding ligand or extracellular protein degrading compound of any one of embodiments 1-10, wherein R⁶⁵ is —CF₃.
17. The ASGPR binding ligand or extracellular protein degrading compound of any one of embodiments 1-10, wherein R⁶⁵ is cyano.
18. The ASGPR binding ligand or extracellular protein degrading compound of any one of embodiments 1-10, wherein R⁶⁵ is F.
19. The ASGPR binding ligand or extracellular protein degrading compound of any one of embodiments 1-10, wherein R⁶⁵ is Cl.
20. The ASGPR binding ligand or extracellular protein degrading compound of any one of embodiments 1-10, wherein R⁶⁵ is Br.
21. The ASGPR binding ligand or extracellular protein degrading compound of any one of embodiments 1-10, wherein R⁶⁵ is haloalkyl.
22. The ASGPR binding ligand or extracellular protein degrading compound of any one of embodiments 1-10, wherein R⁶⁵ is heterocycle.
23. The ASGPR binding ligand or extracellular protein degrading compound of any one of embodiments 1-10, wherein R⁶⁵ is haloalkoxy.
24. The ASGPR binding ligand or extracellular protein degrading compound of any one of embodiments 1-10, wherein R⁶⁵ is C₀-C₆alkyl-OR⁶.
25. The ASGPR binding ligand or extracellular protein degrading compound of any one of embodiments 1-10, wherein R⁶⁵ is C₀-C₆alkyl-SR⁶.
26. The ASGPR binding ligand or extracellular protein degrading compound of any one of embodiments 1-10, wherein R⁶⁵ is C₀-C₆alkyl-NR⁶R⁷.
27. The ASGPR binding ligand or extracellular protein degrading compound of any one of embodiments 1-10, wherein R⁶⁵ is C₀-C₆alkyl-C(O)R³.
28. The ASGPR binding ligand or extracellular protein degrading compound of any one of embodiments 1-10, wherein R⁶⁵ is C₀-C₆alkyl-S(O)R³.
29. The ASGPR binding ligand or extracellular protein degrading compound of any one of embodiments 1-10, wherein R⁶⁵ is C₀-C₆alkyl-C(S)R³.
30. The ASGPR binding ligand or extracellular protein degrading compound of any one of embodiments 1-10, wherein R⁶⁵ is C₀-C₆alkyl-S(O)₂R³.
31. The ASGPR binding ligand or extracellular protein degrading compound of any one of embodiments 1-10, wherein R⁶⁵ is C₀-C₆alkylN₃.
32. The ASGPR binding ligand or extracellular protein degrading compound of any one of embodiments 1-10, wherein R⁶⁵ is C₀-C₆alkyl-cyano.
33. The ASGPR binding ligand or extracellular protein degrading compound of any one of embodiments 24-32, wherein C₀-C₆alkyl is C₀-alkyl (i.e. bond).
34. The ASGPR binding ligand or extracellular protein degrading compound of any one of embodiments 24-32, wherein C₀-C₆alkyl is C₁-alkyl.
35. The ASGPR binding ligand or extracellular protein degrading compound of any one of embodiments 24-32, wherein C₀-C₆alkyl is C₂-alkyl.
36. The ASGPR binding ligand or extracellular protein degrading compound of any one of embodiments 1-10, wherein R⁶⁵ is —N═S(O)(R³)₂.
37. The ASGPR binding ligand or extracellular protein degrading compound of any one of embodiments 1-10, wherein R⁶⁵ is heterocycloalkyl.
38. The ASGPR binding ligand or extracellular protein degrading compound of any one of embodiments 1-10, wherein R⁶⁵ is aryl.
39. The ASGPR binding ligand or extracellular protein degrading compound of any one of embodiments 1-38, wherein R⁶⁶ is not substituted with an optional substituent.
40. The ASGPR binding ligand or extracellular protein degrading compound of any one of embodiments 1-38, wherein R⁶⁶ is substituted with 1 optional substituent.
41. The ASGPR binding ligand or extracellular protein degrading compound of any one of embodiments 1-38, wherein R⁶⁶ is substituted with 2 optional substituents.
42. The ASGPR binding ligand or extracellular protein degrading compound of any one of embodiments 1-38, wherein R⁶⁶ is substituted with 3 optional substituents.
43. The ASGPR binding ligand or extracellular protein degrading compound of any one of embodiments 1-38, wherein R⁶⁶ is hydrogen.
44. The ASGPR binding ligand or extracellular protein degrading compound of any one of embodiments 1-38, wherein R⁶⁶ is —CF₃.
45. The ASGPR binding ligand or extracellular protein degrading compound of any one of embodiments 1-38, wherein R⁶⁶ is cyano.
46. The ASGPR binding ligand or extracellular protein degrading compound of any one of embodiments 1-38, wherein R⁶⁶ is F.
47. The ASGPR binding ligand or extracellular protein degrading compound of any one of embodiments 1-38, wherein R⁶⁶ is Cl.
48. The ASGPR binding ligand or extracellular protein degrading compound of any one of embodiments 1-38, wherein R⁶⁶ is Br.
49. The ASGPR binding ligand or extracellular protein degrading compound of any one of embodiments 1-38, wherein R⁶⁶ is haloalkyl.
50. The ASGPR binding ligand or extracellular protein degrading compound of any one of embodiments 1-38, wherein R⁶⁶ is heterocycle.
51. The ASGPR binding ligand or extracellular protein degrading compound of any one of embodiments 1-38, wherein R⁶⁶ is haloalkoxy.
52. The ASGPR binding ligand or extracellular protein degrading compound of any one of embodiments 1-38, wherein R⁶⁶ is C₀-C₆alkyl-OR⁶.
53. The ASGPR binding ligand or extracellular protein degrading compound of any one of embodiments 1-38, wherein R⁶⁶ is C₀-C₆alkyl-SR⁶.
54. The ASGPR binding ligand or extracellular protein degrading compound of any one of embodiments 1-38, wherein R⁶⁶ is C₀-C₆alkyl-NR⁶R⁷.
55. The ASGPR binding ligand or extracellular protein degrading compound of any one of embodiments 1-38, wherein R⁶⁶ is C₀-C₆alkyl-C(O)R³.
56. The ASGPR binding ligand or extracellular protein degrading compound of any one of embodiments 1-38, wherein R⁶⁶ is C₀-C₆alkyl-S(O)R³.
57. The ASGPR binding ligand or extracellular protein degrading compound of any one of embodiments 1-38, wherein R⁶⁶ is C₀-C₆alkyl-C(S)R³.
58. The ASGPR binding ligand or extracellular protein degrading compound of any one of embodiments 1-38, wherein R⁶⁶ is C₀-C₆alkyl-S(O)₂R³.
59. The ASGPR binding ligand or extracellular protein degrading compound of any one of embodiments 1-38, wherein R⁶⁶ is C₀-C₆alkylN₃.
60. The ASGPR binding ligand or extracellular protein degrading compound of any one of embodiments 1-38, wherein R⁶⁶ is C₀-C₆alkyl-cyano.
61. The ASGPR binding ligand or extracellular protein degrading compound of any one of embodiments 52-60, wherein C₀-C₆alkyl is C₀-alkyl (i.e. bond).
62. The ASGPR binding ligand or extracellular protein degrading compound of any one of embodiments 52-60, wherein C₀-C₆alkyl is C₁-alkyl.
63. The ASGPR binding ligand or extracellular protein degrading compound of any one of embodiments 52-60, wherein C₀-C₆alkyl is C₂-alkyl.
64. The ASGPR binding ligand or extracellular protein degrading compound of any one of embodiments 1-38, wherein R⁶⁶ is —N═S(O)(R³)₂.
65. The ASGPR binding ligand or extracellular protein degrading compound of any one of embodiments 1-38, wherein R⁶⁶ is heterocycloalkyl.
66. The ASGPR binding ligand or extracellular protein degrading compound of any one of embodiments 1-38, wherein R⁶⁶ is aryl.
67. The ASGPR binding ligand or extracellular protein degrading compound of any one of embodiments 1-66, wherein R⁶⁷ is not substituted with an optional substituent.
68. The ASGPR binding ligand or extracellular protein degrading compound of any one of embodiments 1-66, wherein R⁶⁷ is substituted with 1 optional substituent.
69. The ASGPR binding ligand or extracellular protein degrading compound of any one of embodiments 1-66, wherein R⁶⁷ is substituted with 2 optional substituents.
70. The ASGPR binding ligand or extracellular protein degrading compound of any one of embodiments 1-66, wherein R⁶⁷ is substituted with 3 optional substituents.
71. The ASGPR binding ligand or extracellular protein degrading compound of any one of embodiments 1-66, wherein R⁶⁷ is hydrogen.
72. The ASGPR binding ligand or extracellular protein degrading compound of any one of embodiments 1-66, wherein R⁶⁷ is —CF₃.
73. The ASGPR binding ligand or extracellular protein degrading compound of any one of embodiments 1-66, wherein R⁶⁷ is cyano.
74. The ASGPR binding ligand or extracellular protein degrading compound of any one of embodiments 1-66, wherein R⁶⁷ is F.
75. The ASGPR binding ligand or extracellular protein degrading compound of any one of embodiments 1-66, wherein R⁶⁷ is Cl.
76. The ASGPR binding ligand or extracellular protein degrading compound of any one of embodiments 1-66, wherein R⁶⁷ is Br.
77. The ASGPR binding ligand or extracellular protein degrading compound of any one of embodiments 1-66, wherein R⁶⁷ is haloalkyl.
78. The ASGPR binding ligand or extracellular protein degrading compound of any one of embodiments 1-66, wherein R⁶⁷ is heterocycle.
79. The ASGPR binding ligand or extracellular protein degrading compound of any one of embodiments 1-66, wherein R⁶⁷ is haloalkoxy.
80. The ASGPR binding ligand or extracellular protein degrading compound of any one of embodiments 1-66, wherein R⁶⁷ is C₀-C₆alkyl-OR⁶.
81. The ASGPR binding ligand or extracellular protein degrading compound of any one of embodiments 1-66, wherein R⁶⁷ is C₀-C₆alkyl-SR⁶.
82. The ASGPR binding ligand or extracellular protein degrading compound of any one of embodiments 1-66, wherein R⁶⁷ is C₀-C₆alkyl-NR⁶R⁷.
83. The ASGPR binding ligand or extracellular protein degrading compound of any one of embodiments 1-66, wherein R⁶⁷ is C₀-C₆alkyl-C(O)R³.
84. The ASGPR binding ligand or extracellular protein degrading compound of any one of embodiments 1-66, wherein R⁶⁷ is C₀-C₆alkyl-S(O)R³.
85. The ASGPR binding ligand or extracellular protein degrading compound of any one of embodiments 1-66, wherein R⁶⁷ is C₀-C₆alkyl-C(S)R³.
86. The ASGPR binding ligand or extracellular protein degrading compound of any one of embodiments 1-66, wherein R⁶⁷ is C₀-C₆alkyl-S(O)₂R³.
87. The ASGPR binding ligand or extracellular protein degrading compound of any one of embodiments 1-66, wherein R⁶⁷ is C₀-C₆alkylN₃.
88. The ASGPR binding ligand or extracellular protein degrading compound of any one of embodiments 1-66, wherein R⁶⁷ is C₀-C₆alkyl-cyano.
89. The ASGPR binding ligand or extracellular protein degrading compound of any one of embodiments 80-88, wherein C₀-C₆alkyl is C₀-alkyl (i.e. bond).
90. The ASGPR binding ligand or extracellular protein degrading compound of any one of embodiments 80-88, wherein C₀-C₆alkyl is C₁-alkyl.
91. The ASGPR binding ligand or extracellular protein degrading compound of any one of embodiments 80-88, wherein C₀-C₆alkyl is C₂-alkyl.
92. The ASGPR binding ligand or extracellular protein degrading compound of any one of embodiments 1-66, wherein R⁶⁷ is —N═S(O)(R³)₂.
93. The ASGPR binding ligand or extracellular protein degrading compound of any one of embodiments 1-66, wherein R⁶⁷ is heterocycloalkyl.
94. The ASGPR binding ligand or extracellular protein degrading compound of any one of embodiments 1-66, wherein R⁶⁷ is aryl.
95. The ASGPR binding ligand or extracellular protein degrading compound of embodiment 1, wherein

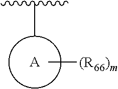

is

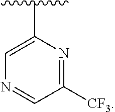 [SMILES: *c1cncc(C(F)(F)F)n1]

96. The ASGPR binding ligand or extracellular protein degrading compound of embodiment 1, wherein

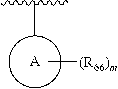

is

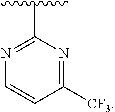 [SMILES: *c1nccc(C(F)(F)F)n1]

97. The ASGPR binding ligand or extracellular protein degrading compound of embodiment 1, wherein

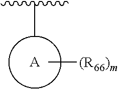

is

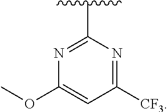 [SMILES: *c1nc(OC)cc(C(F)(F)F)n1]

98. The ASGPR binding ligand or extracellular protein degrading compound of embodiment 1, wherein

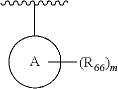

is

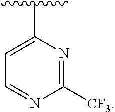 [SMILES: *c1ccnc(C(F)(F)F)n1]

99. The ASGPR binding ligand or extracellular protein degrading compound of embodiment 1, wherein

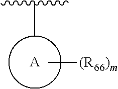

is

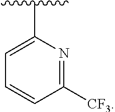 [SMILES: *c1cccc(C(F)(F)F)n1]

100. The ASGPR binding ligand or extracellular protein degrading compound of embodiment 1, wherein

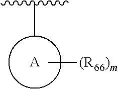

is

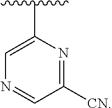 [SMILES: *c1cncc(C#N)n1]

101. The ASGPR binding ligand or extracellular protein degrading compound of embodiment 1, wherein

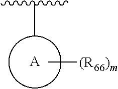

is

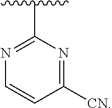 [SMILES: *c1nccc(C#N)n1]

102. The ASGPR binding ligand or extracellular protein degrading compound of embodiment 1, wherein

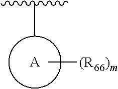

is

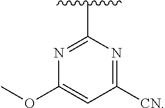 [SMILES: *c1nc(C#N)cc(OC)n1]

103. The ASGPR binding ligand or extracellular protein degrading compound of embodiment 1, wherein

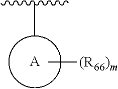

is

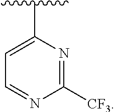 [SMILES: *c1ccnc(C(F)(F)F)n1]

104. The ASGPR binding ligand or extracellular protein degrading compound of embodiment 1, wherein

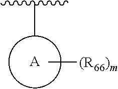

is

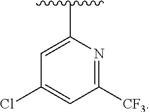 [SMILES: *c1cc(Cl)cc(C(F)(F)F)n1]

105. The ASGPR binding ligand or extracellular protein degrading compound of embodiment 1, wherein

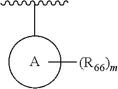

is

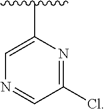 [SMILES: *c1cncc(Cl)n1]

106. The ASGPR binding ligand or extracellular protein degrading compound of embodiment 1, wherein

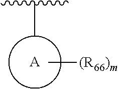

is

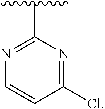 [SMILES: *c1nccc(Cl)n1]

107. The ASGPR binding ligand or extracellular protein degrading compound of embodiment 1, wherein

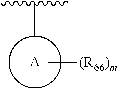

is

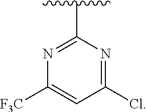 [SMILES: *c1nc(Cl)cc(C(F)(F)F)n1]

108. The ASGPR binding ligand or extracellular protein degrading compound of embodiment 1, wherein

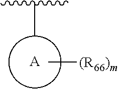

is

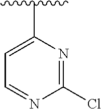 [SMILES: *c1ccnc(Cl)n1]

109. The ASGPR binding ligand or extracellular protein degrading compound of embodiment 1, wherein

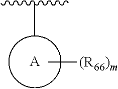

is

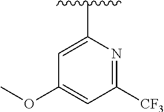 [SMILES: *c1cc(OC)cc(C(F)(F)F)n1]

110. The ASGPR binding ligand or extracellular protein degrading compound of embodiment 1, wherein

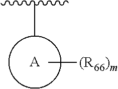

is

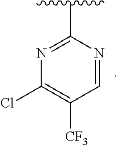 [SMILES: *c1ncc(C(F)(F)F)c(Cl)n1]

111. The ASGPR binding ligand or extracellular protein degrading compound of embodiment 1, wherein

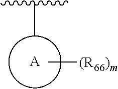

is

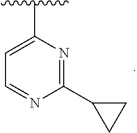 [SMILES: *c1ccnc(C2CC2)n1]

112. The ASGPR binding ligand or extracellular protein degrading compound of embodiment 1, wherein

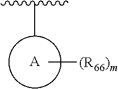

is

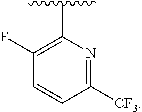 [SMILES: *c1nc(C(F)(F)F)ccc1F]

113. The ASGPR binding ligand or extracellular protein degrading compound of embodiment 1, wherein

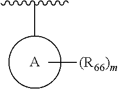

is

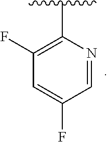 [SMILES: *c1ncc(F)cc1F]

114. The ASGPR binding ligand or extracellular protein degrading compound of embodiment 1, wherein

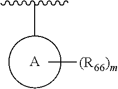

is

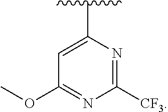 [SMILES: *c1cc(OC)nc(C(F)(F)F)n1]

115. The ASGPR binding ligand or extracellular protein degrading compound of embodiment 1, wherein

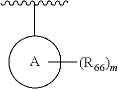

is

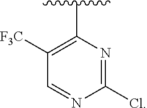 [SMILES: *c1nc(Cl)ncc1C(F)(F)F]

116. The ASGPR binding ligand or extracellular protein degrading compound of embodiment 1, wherein

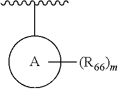

is

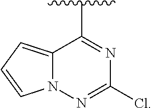 [SMILES: *c1nc(Cl)nn2cccc12]

117. The ASGPR binding ligand or extracellular protein degrading compound of any one of embodiments 1-116, wherein R¹⁰ is hydrogen.
118. The ASGPR binding ligand or extracellular protein degrading compound of any one of embodiments 1-116, wherein R¹⁰ is alkyl.
119. The ASGPR binding ligand or extracellular protein degrading compound of any one of embodiments 1-116, wherein R¹⁰ is methyl.
120. The ASGPR binding ligand or extracellular protein degrading compound of any one of embodiments 1-116, wherein R¹⁰ is deuterium.
121. The ASGPR binding ligand or extracellular protein degrading compound of any one of embodiments 1-116, wherein R¹⁰ is C(O)R³.
122. The ASGPR binding ligand or extracellular protein degrading compound of any one of embodiments 1-116, wherein R¹⁰ is arylalkyl.
123. The ASGPR binding ligand or extracellular protein degrading compound of any one of embodiments 1-122, wherein R⁷⁵, R⁷⁶, R⁷⁷, and R⁷⁸ are independently selected at each occurrence from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R⁹⁹.
124. The ASGPR binding ligand or extracellular protein degrading compound of any one of embodiments 1-123, wherein B is aryl.
125. The ASGPR binding ligand or extracellular protein degrading compound of any one of embodiments 1-123, wherein B is heterocycle.
126. The ASGPR binding ligand or extracellular protein degrading compound of any one of embodiments 1-123, wherein B is cycloalkyl.
127. The ASGPR binding ligand or extracellular protein degrading compound of any one of embodiments 1-123, wherein B is heteroaryl.
128. The ASGPR binding ligand or extracellular protein degrading compound of any one of embodiments 1-123, wherein B has one optional substituent.
129. The ASGPR binding ligand or extracellular protein degrading compound of any one of embodiments 1-123, wherein B has two optional substituents.
130. The ASGPR binding ligand or extracellular protein degrading compound of any one of embodiments 1-123, wherein B has three optional substituents.
131. The ASGPR binding ligand or extracellular protein degrading compound of any one of embodiments 1-123, wherein B has four optional substituents.
132. The ASGPR binding ligand or extracellular protein degrading compound of any one of embodiments 1-123, wherein

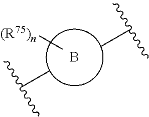

is

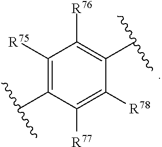

133. The ASGPR binding ligand or extracellular protein degrading compound of any one of embodiments 1-123, wherein

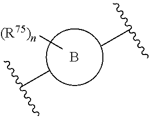

is

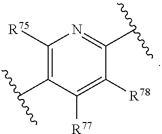

134. The ASGPR binding ligand or extracellular protein degrading compound of any one of embodiments 1-123, wherein

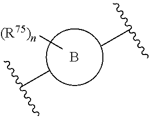

is

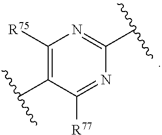

135. The ASGPR binding ligand or extracellular protein degrading compound of any one of embodiments 1-123, wherein

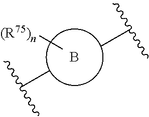

is

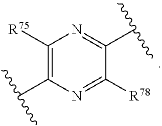

136. The ASGPR binding ligand or extracellular protein degrading compound of any one of embodiments 1-123, wherein

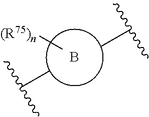

is

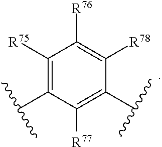

137. The ASGPR binding ligand or extracellular protein degrading compound of any one of embodiments 1-123, wherein

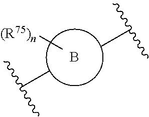

is

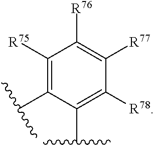

138. The ASGPR binding ligand or extracellular protein degrading compound of any one of embodiments 1-123, wherein

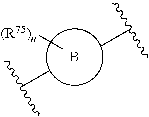

is

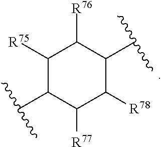

139. The ASGPR binding ligand or extracellular protein degrading compound of any one of embodiments 1-123, wherein

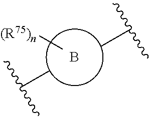

is

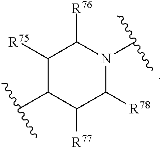

140. The ASGPR binding ligand or extracellular protein degrading compound of any one of embodiments 1-123, wherein

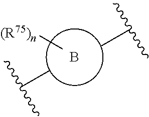

is

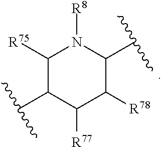

141. The ASGPR binding ligand or extracellular protein degrading compound of any one of embodiments 1-123, wherein

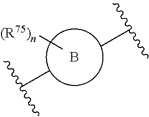

is

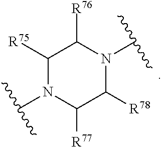

142. The ASGPR binding ligand or extracellular protein degrading compound of any one of embodiments 1-123, wherein

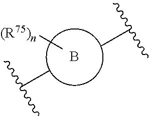

is

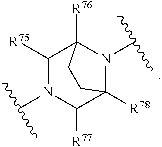

143. The ASGPR binding ligand or extracellular protein degrading compound of any one of embodiments 1-142, wherein R⁷⁵ is hydrogen.
144. The ASGPR binding ligand or extracellular protein degrading compound of any one of embodiments 1-142, wherein R⁷⁵ is —CF₃.
145. The ASGPR binding ligand or extracellular protein degrading compound of any one of embodiments 1-142, wherein R⁷⁵ is cyano.
146. The ASGPR binding ligand or extracellular protein degrading compound of any one of embodiments 1-142, wherein R⁷⁵ is F.
147. The ASGPR binding ligand or extracellular protein degrading compound of any one of embodiments 1-142, wherein R⁷⁵ is Cl.
148. The ASGPR binding ligand or extracellular protein degrading compound of any one of embodiments 1-142, wherein R⁷⁵ is Br.
149. The ASGPR binding ligand or extracellular protein degrading compound of any one of embodiments 1-142, wherein R⁷⁵ is haloalkyl.
150. The ASGPR binding ligand or extracellular protein degrading compound of any one of embodiments 1-142, wherein R⁷⁵ is heterocycle.
151. The ASGPR binding ligand or extracellular protein degrading compound of any one of embodiments 1-142, wherein R⁷⁵ is haloalkoxy.
152. The ASGPR binding ligand or extracellular protein degrading compound of any one of embodiments 1-142, wherein R⁷⁵ is C₀-C₆alkyl-OR⁶.
153. The ASGPR binding ligand or extracellular protein degrading compound of any one of embodiments 1-142, wherein R⁷⁵ is C₀-C₆alkyl-SR⁶.
154. The ASGPR binding ligand or extracellular protein degrading compound of any one of embodiments 1-142, wherein R⁷⁵ is C₀-C₆alkyl-NR⁶R⁷.
155. The ASGPR binding ligand or extracellular protein degrading compound of any one of embodiments 1-142, wherein R⁷⁵ is C₀-C₆alkyl-C(O)R³.
156. The ASGPR binding ligand or extracellular protein degrading compound of any one of embodiments 1-142, wherein R⁷⁵ is C₀-C₆alkyl-S(O)R³.
157. The ASGPR binding ligand or extracellular protein degrading compound of any one of embodiments 1-142, wherein R⁷⁵ is C₀-C₆alkyl-C(S)R³.
158. The ASGPR binding ligand or extracellular protein degrading compound of any one of embodiments 1-142, wherein R⁷⁵ is C₀-C₆alkyl-S(O)₂R³.
159. The ASGPR binding ligand or extracellular protein degrading compound of any one of embodiments 1-142, wherein R⁷⁵ is C₀-C₆alkylN₃.
160. The ASGPR binding ligand or extracellular protein degrading compound of any one of embodiments 1-142, wherein R⁷⁵ is C₀-C₆alkyl-cyano.
161. The ASGPR binding ligand or extracellular protein degrading compound of any one of embodiments 152-160, wherein C₀-C₆alkyl is C₀-alkyl (i.e. bond).
162. The ASGPR binding ligand or extracellular protein degrading compound of any one of embodiments 152-160, wherein C₀-C₆alkyl is C₁-alkyl.
163. The ASGPR binding ligand or extracellular protein degrading compound of any one of embodiments 152-160, wherein C₀-C₆alkyl is C₂-alkyl.
164. The ASGPR binding ligand or extracellular protein degrading compound of any one of embodiments 1-142, wherein R⁷⁵ is —N═S(O)(R³)₂.
165. The ASGPR binding ligand or extracellular protein degrading compound of any one of embodiments 1-142, wherein R⁷⁵ is heterocycloalkyl.
166. The ASGPR binding ligand or extracellular protein degrading compound of any one of embodiments 1-142, wherein R⁷⁵ is aryl.
167. The ASGPR binding ligand or extracellular protein degrading compound of any one of embodiments 152-166, wherein R⁷⁵ is not substituted with an optional substituent.
168. The ASGPR binding ligand or extracellular protein degrading compound of any one of embodiments 152-166, wherein R⁷⁵ is substituted with 1 optional substituent.
169. The ASGPR binding ligand or extracellular protein degrading compound of any one of embodiments 152-166, wherein R⁷⁵ is substituted with 2 optional substituents.
170. The ASGPR binding ligand or extracellular protein degrading compound of any one of embodiments 152-166, wherein R⁷⁵ is substituted with 3 optional substituents.
171. The ASGPR binding ligand or extracellular protein degrading compound of any one of embodiments 1-170, wherein R⁷⁶ is hydrogen.
172. The ASGPR binding ligand or extracellular protein degrading compound of any one of embodiments 1-170, wherein R⁷⁶ is —CF₃.
173. The ASGPR binding ligand or extracellular protein degrading compound of any one of embodiments 1-170, wherein R⁷⁶ is cyano.
174. The ASGPR binding ligand or extracellular protein degrading compound of any one of embodiments 1-170, wherein R⁷⁶ is F.
175. The ASGPR binding ligand or extracellular protein degrading compound of any one of embodiments 1-170, wherein R⁷⁶ is Cl.
176. The ASGPR binding ligand or extracellular protein degrading compound of any one of embodiments 1-170, wherein R⁷⁶ is Br.
177. The ASGPR binding ligand or extracellular protein degrading compound of any one of embodiments 1-170, wherein R⁷⁶ is haloalkyl.
178. The ASGPR binding ligand or extracellular protein degrading compound of any one of embodiments 1-170, wherein R⁷⁶ is heterocycle.
179. The ASGPR binding ligand or extracellular protein degrading compound of any one of embodiments 1-170, wherein R⁷⁶ is haloalkoxy.
180. The ASGPR binding ligand or extracellular protein degrading compound of any one of embodiments 1-170, wherein R⁷⁶ is C₀-C₆alkyl-OR⁶.
181. The ASGPR binding ligand or extracellular protein degrading compound of any one of embodiments 1-170, wherein R⁷⁶ is C₀-C₆alkyl-SR⁶.
182. The ASGPR binding ligand or extracellular protein degrading compound of any one of embodiments 1-170, wherein R⁷⁶ is C₀-C₆alkyl-NR⁶R⁷.
183. The ASGPR binding ligand or extracellular protein degrading compound of any one of embodiments 1-170, wherein R⁷⁶ is C₀-C₆alkyl-C(O)R³.
184. The ASGPR binding ligand or extracellular protein degrading compound of any one of embodiments 1-170, wherein R⁷⁶ is C₀-C₆alkyl-S(O)R³.
185. The ASGPR binding ligand or extracellular protein degrading compound of any one of embodiments 1-170, wherein R⁷⁶ is C₀-C₆alkyl-C(S)R³.
186. The ASGPR binding ligand or extracellular protein degrading compound of any one of embodiments 1-170, wherein R⁷⁶ is C₀-C₆alkyl-S(O)₂R³.
187. The ASGPR binding ligand or extracellular protein degrading compound of any one of embodiments 1-170, wherein R⁷⁶ is C₀-C₆alkylN₃.
188. The ASGPR binding ligand or extracellular protein degrading compound of any one of embodiments 1-170, wherein R⁷⁶ is C₀-C₆alkyl-cyano.
189. The ASGPR binding ligand or extracellular protein degrading compound of any one of embodiments 180-188, wherein C₀-C₆alkyl is C₀-alkyl (i.e. bond).
190. The ASGPR binding ligand or extracellular protein degrading compound of any one of embodiments 180-188, wherein C₀-C₆alkyl is C₁-alkyl.
191. The ASGPR binding ligand or extracellular protein degrading compound of any one of embodiments 180-188, wherein C₀-C₆alkyl is C₂-alkyl.
192. The ASGPR binding ligand or extracellular protein degrading compound of any one of embodiments 1-170, wherein R⁷⁶ is —N═S(O)(R³)₂.
193. The ASGPR binding ligand or extracellular protein degrading compound of any one of embodiments 1-170, wherein R⁷⁶ is heterocycloalkyl.
194. The ASGPR binding ligand or extracellular protein degrading compound of any one of embodiments 1-170, wherein R⁷⁶ is aryl.
195. The ASGPR binding ligand or extracellular protein degrading compound of any one of embodiments 1-194, wherein R⁷⁷ is hydrogen.
196. The ASGPR binding ligand or extracellular protein degrading compound of any one of embodiments 1-194, wherein R⁷⁷ is —CF₃.
197. The ASGPR binding ligand or extracellular protein degrading compound of any one of embodiments 1-194, wherein R⁷⁷ is cyano.
198. The ASGPR binding ligand or extracellular protein degrading compound of any one of embodiments 1-194, wherein R⁷⁷ is F.
199. The ASGPR binding ligand or extracellular protein degrading compound of any one of embodiments 1-194, wherein R⁷⁷ is Cl.
200. The ASGPR binding ligand or extracellular protein degrading compound of any one of embodiments 1-194, wherein R⁷⁷ is Br.
201. The ASGPR binding ligand or extracellular protein degrading compound of any one of embodiments 1-194, wherein R⁷⁷ is haloalkyl.
202. The ASGPR binding ligand or extracellular protein degrading compound of any one of embodiments 1-194, wherein R⁷⁷ is heterocycle.
203. The ASGPR binding ligand or extracellular protein degrading compound of any one of embodiments 1-194, wherein R⁷⁷ is haloalkoxy.
204. The ASGPR binding ligand or extracellular protein degrading compound of any one of embodiments 1-194, wherein R⁷⁷ is C₀-C₆alkyl-OR⁶.
205. The ASGPR binding ligand or extracellular protein degrading compound of any one of embodiments 1-194, wherein R⁷⁷ is C₀-C₆alkyl-SR⁶.
206. The ASGPR binding ligand or extracellular protein degrading compound of any one of embodiments 1-194, wherein R⁷⁷ is C₀-C₆alkyl-NR⁶R⁷.
207. The ASGPR binding ligand or extracellular protein degrading compound of any one of embodiments 1-194, wherein R⁷⁷ is C₀-C₆alkyl-C(O)R³.
208. The ASGPR binding ligand or extracellular protein degrading compound of any one of embodiments 1-194, wherein R⁷⁷ is C₀-C₆alkyl-S(O)R³.
209. The ASGPR binding ligand or extracellular protein degrading compound of any one of embodiments 1-194, wherein R⁷⁷ is C₀-C₆alkyl-C(S)R³.
210. The ASGPR binding ligand or extracellular protein degrading compound of any one of embodiments 1-194, wherein R⁷⁷ is C₀-C₆alkyl-S(O)₂R³.
211. The ASGPR binding ligand or extracellular protein degrading compound of any one of embodiments 1-194, wherein R⁷⁷ is C₀-C₆alkylN₃.
212. The ASGPR binding ligand or extracellular protein degrading compound of any one of embodiments 1-194, wherein R⁷⁷ is C₀-C₆alkyl-cyano.
213. The ASGPR binding ligand or extracellular protein degrading compound of any one of embodiments 204-212, wherein C₀-C₆alkyl is C₀-alkyl (i.e. bond).
214. The ASGPR binding ligand or extracellular protein degrading compound of any one of embodiments 204-212, wherein C₀-C₆alkyl is C₁-alkyl.
215. The ASGPR binding ligand or extracellular protein degrading compound of any one of embodiments 204-212, wherein C₀-C₆alkyl is C₂-alkyl.
216. The ASGPR binding ligand or extracellular protein degrading compound of any one of embodiments 1-194, wherein R⁷⁷ is —N═S(O)(R³)₂.
217. The ASGPR binding ligand or extracellular protein degrading compound of any one of embodiments 1-194, wherein R⁷⁷ is heterocycloalkyl.
218. The ASGPR binding ligand or extracellular protein degrading compound of any one of embodiments 1-194, wherein R⁷⁷ is aryl.
219. The ASGPR binding ligand or extracellular protein degrading compound of any one of embodiments 1-218, wherein R⁷⁸ is hydrogen.
220. The ASGPR binding ligand or extracellular protein degrading compound of any one of embodiments 1-218, wherein R⁷⁸ is —CF₃.
221. The ASGPR binding ligand or extracellular protein degrading compound of any one of embodiments 1-218, wherein R⁷⁸ is cyano.
222. The ASGPR binding ligand or extracellular protein degrading compound of any one of embodiments 1-218, wherein R⁷⁸ is F.
223. The ASGPR binding ligand or extracellular protein degrading compound of any one of embodiments 1-218, wherein R⁷⁸ is Cl.
224. The ASGPR binding ligand or extracellular protein degrading compound of any one of embodiments 1-218, wherein R⁷⁸ is Br.
225. The ASGPR binding ligand or extracellular protein degrading compound of any one of embodiments 1-218, wherein R⁷⁸ is haloalkyl.
226. The ASGPR binding ligand or extracellular protein degrading compound of any one of embodiments 1-218, wherein R⁷⁸ is heterocycle.
227. The ASGPR binding ligand or extracellular protein degrading compound of any one of embodiments 1-218, wherein R⁷⁸ is haloalkoxy.
228. The ASGPR binding ligand or extracellular protein degrading compound of any one of embodiments 1-218, wherein R⁷⁸ is C₀-C₆alkyl-OR⁶.
229. The ASGPR binding ligand or extracellular protein degrading compound of any one of embodiments 1-218, wherein R⁷⁸ is C₀-C₆alkyl-SR⁶.
230. The ASGPR binding ligand or extracellular protein degrading compound of any one of embodiments 1-218, wherein R⁷⁸ is C₀-C₆alkyl-NR⁶R⁷.
231. The ASGPR binding ligand or extracellular protein degrading compound of any one of embodiments 1-218, wherein R⁷⁸ is C₀-C₆alkyl-C(O)R³.
232. The ASGPR binding ligand or extracellular protein degrading compound of any one of embodiments 1-218, wherein R⁷⁸ is C₀-C₆alkyl-S(O)R³.
233. The ASGPR binding ligand or extracellular protein degrading compound of any one of embodiments 1-218, wherein R⁷⁸ is C₀-C₆alkyl-C(S)R³.
234. The ASGPR binding ligand or extracellular protein degrading compound of any one of embodiments 1-218, wherein R⁷⁸ is C₀-C₆alkyl-S(O)₂R³.
235. The ASGPR binding ligand or extracellular protein degrading compound of any one of embodiments 1-218, wherein R⁷⁸ is C₀-C₆alkylN₃.
236. The ASGPR binding ligand or extracellular protein degrading compound of any one of embodiments 1-218, wherein R⁷⁸ is C₀-C₆alkyl-cyano.
237. The ASGPR binding ligand or extracellular protein degrading compound of any one of embodiments 228-236, wherein C₀-C₆alkyl is C₀-alkyl (i.e. bond).
238. The ASGPR binding ligand or extracellular protein degrading compound of any one of embodiments 228-236, wherein C₀-C₆alkyl is C₁-alkyl.
239. The ASGPR binding ligand or extracellular protein degrading compound of any one of embodiments 228-236, wherein C₀-C₆alkyl is C₂-alkyl.
240. The ASGPR binding ligand or extracellular protein degrading compound of any one of embodiments 1-218, wherein R⁷⁸ is —N═S(O)(R³)₂.
241. The ASGPR binding ligand or extracellular protein degrading compound of any one of embodiments 1-218, wherein R⁷⁸ is heterocycloalkyl.
242. The ASGPR binding ligand or extracellular protein degrading compound of any one of embodiments 1-218, wherein R⁷⁸ is aryl.
243. The ASGPR binding ligand or extracellular protein degrading compound of any one of embodiments 1-242, wherein C is aryl.
244. The ASGPR binding ligand or extracellular protein degrading compound of any one of embodiments 1-242, wherein C is heteroaryl.
245. The ASGPR binding ligand or extracellular protein degrading compound of any one of embodiments 1-242, wherein C is cycloalkyl.
246. The ASGPR binding ligand or extracellular protein degrading compound of any one of embodiments 1-242, wherein C is bicycle.
247. The ASGPR binding ligand or extracellular protein degrading compound of any one of embodiments 1-242, wherein C is heterocycle.
248. The ASGPR binding ligand or extracellular protein degrading compound of any one of embodiments 1-248, wherein C is substituted by one optional substituent.
249. The ASGPR binding ligand or extracellular protein degrading compound of any one of embodiments 1-248, wherein C is substituted by two optional substituents.
250. The ASGPR binding ligand or extracellular protein degrading compound of any one of embodiments 1-248, wherein C is substituted by three optional substituents.
251. The ASGPR binding ligand or extracellular protein degrading compound of any one of embodiments 1-250, wherein C is

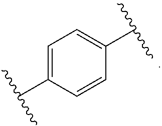 [SMILES: *c1ccc(*)cc1]

252. The ASGPR binding ligand or extracellular protein degrading compound of any one of embodiments 1-250, wherein C is

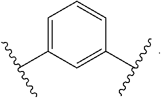 [SMILES: *c1cccc(*)c1]

253. The ASGPR binding ligand or extracellular protein degrading compound of any one of embodiments 1-250, wherein C is

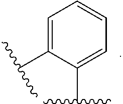 [SMILES: *c1ccccc1*]

254. The ASGPR binding ligand or extracellular protein degrading compound of any one of embodiments 1-250, wherein C is

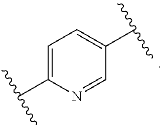 [SMILES: *c1ccc(*)nc1]

255. The ASGPR binding ligand or extracellular protein degrading compound of any one of embodiments 1-250, wherein C is

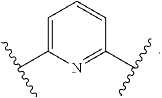 [SMILES: *c1cccc(*)n1]

256. The ASGPR binding ligand or extracellular protein degrading compound of any one of embodiments 1-250, wherein C is

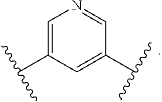 [SMILES: *c1cncc(*)c1]

257. The ASGPR binding ligand or extracellular protein degrading compound of any one of embodiments 1-250, wherein C is

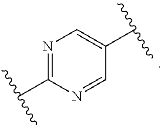 [SMILES: *c1cnc(*)nc1]

258. The ASGPR binding ligand or extracellular protein degrading compound of any one of embodiments 1-250, wherein C is

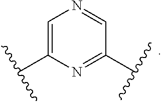 [SMILES: *c1cncc(*)n1]

259. The ASGPR binding ligand or extracellular protein degrading compound of any one of embodiments 1-250, wherein C is

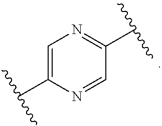 [SMILES: *c1cnc(*)cn1]

260. The ASGPR binding ligand or extracellular protein degrading compound of any one of embodiments 1-250, wherein C is

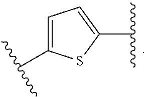 [SMILES: *c1ccc(*)s1]

261. The ASGPR binding ligand or extracellular protein degrading compound of any one of embodiments 1-250, wherein C is

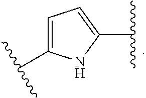 [SMILES: *c1ccc(*)[nH]1]

262. The ASGPR binding ligand or extracellular protein degrading compound of any one of embodiments 1-250, wherein C is

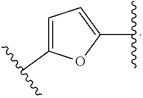 [SMILES: *c1ccc(*)o1]

263. The ASGPR binding ligand or extracellular protein degrading compound of any of embodiments 1-250, wherein C is

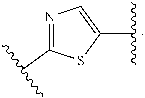 [SMILES: *c1cnc(*)s1]

264. The ASGPR binding ligand or extracellular protein degrading compound of any one of embodiments 1-250, wherein C is

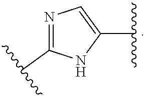 [SMILES: *c1cnc(*)[nH]1]

265. The ASGPR binding ligand or extracellular protein degrading compound of any one of embodiments 1-250, wherein C is

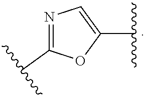 [SMILES: *c1cnc(*)o1]

266. The ASGPR binding ligand or extracellular protein degrading compound of any one of embodiments 1-250, wherein C is

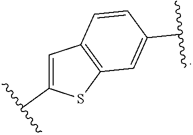 [SMILES: *c1ccc2cc(*)sc2c1]

267. The ASGPR binding ligand or extracellular protein degrading compound of any one of embodiments 1-250, wherein C is

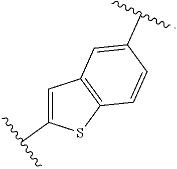 [SMILES: *c1ccc2sc(*)cc2c1]

268. The ASGPR binding ligand or extracellular protein degrading compound of any one of embodiments 1-250, wherein C is

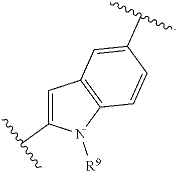

269. The ASGPR binding ligand or extracellular protein degrading compound of any one of embodiments 1-250, wherein C is

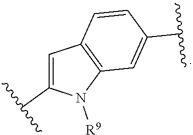

270. The ASGPR binding ligand or extracellular protein degrading compound of any one of embodiments 1-250, wherein C is

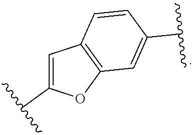 [SMILES: *c1ccc2cc(*)oc2c1]

271. The ASGPR binding ligand or extracellular protein degrading compound of any one of embodiments 1-250, wherein C is

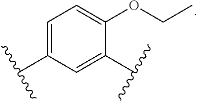 [SMILES: *c1ccc(OCC)c(*)c1]

272. The ASGPR binding ligand or extracellular protein degrading compound of any one of embodiments 1-250, wherein C is

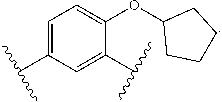 [SMILES: *c1ccc(OC2CCCC2)c(*)c1]

273. The ASGPR binding ligand or extracellular protein degrading compound of any one of embodiments 1-250, wherein C is

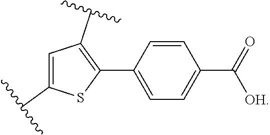 [SMILES: *c1cc(*)c(-c2ccc(C(=O)O)cc2)s1]

### Embodiments of ASGPR Binding Ligand

In certain aspects of the invention, an ASGPR Binding Ligand selected from the following formulas can be used independently from a heterobifunctional molecule:

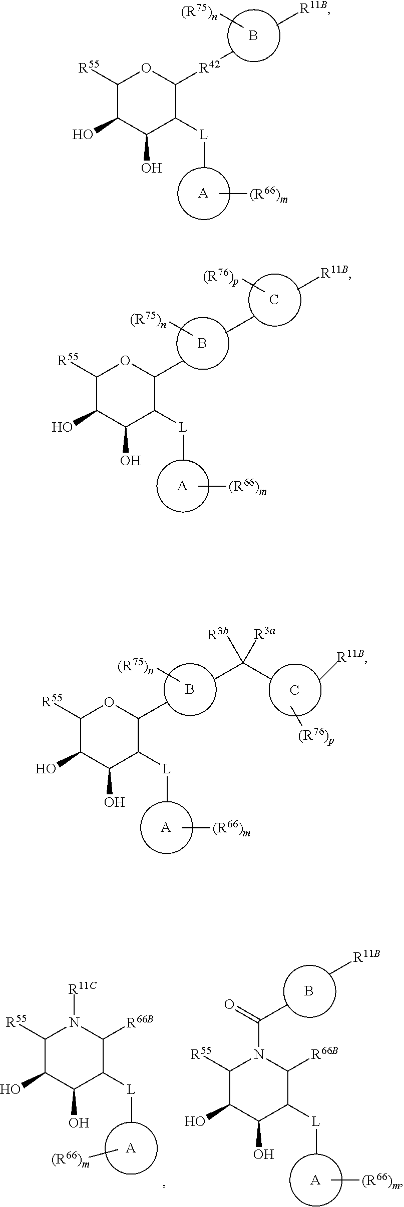

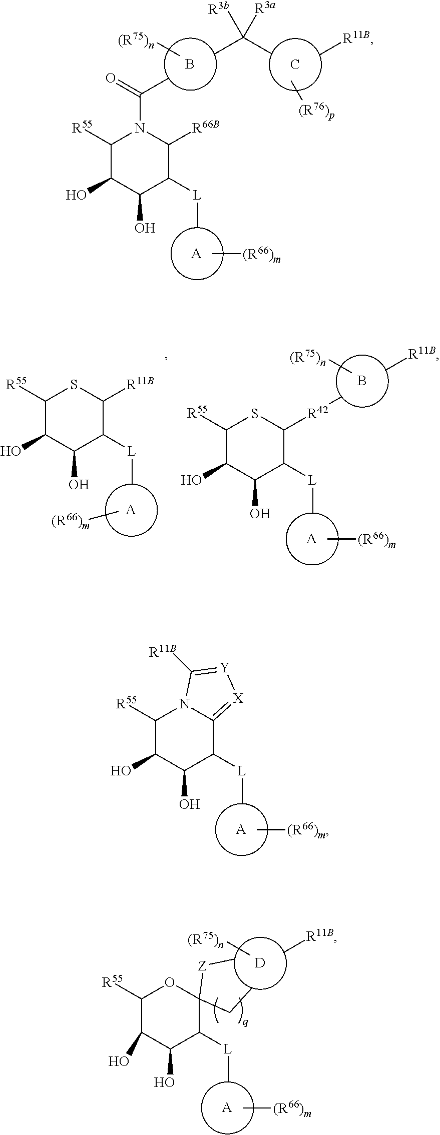

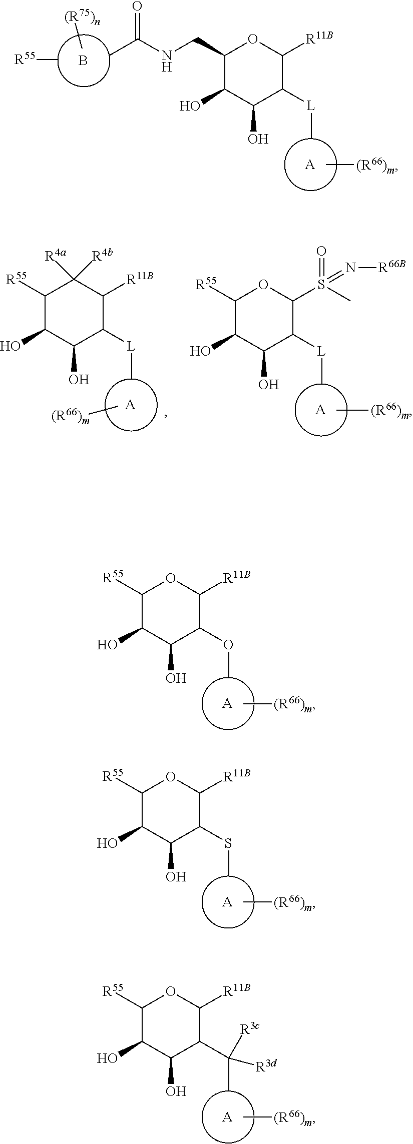

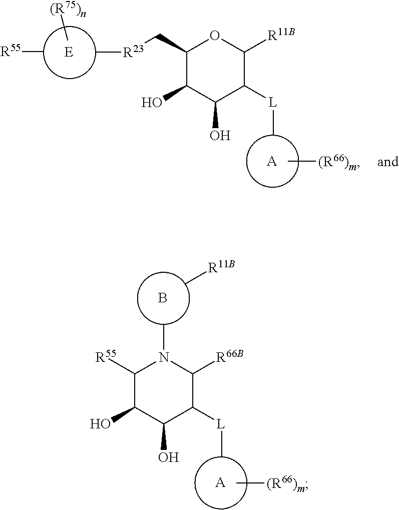

or a salt thereof,
wherein:
R^{11B}, R⁵⁵, and R^{66B} are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkylN₃, and R¹¹⁰ each of which is optionally substituted with 1, 2, 3, or 4 substituents independently selected at each occurrence from R⁹⁹;
R^{11C} is selected from hydrogen, alkyl, alkenyl, alkynyl, haloalkyl, —C(O)R³, —S(O)R³, —C(S)R³, —S(O)₂R³, and R¹¹⁰;
and wherein in typical embodiments where R^{11B} is attached to a nitrogen then R^{11B} is selected from hydrogen, alkyl, alkenyl, alkynyl, haloalkyl, aryl, arylalkyl, heteroaryl, heteroarylalkyl, heterocycle, heterocycloalkyl, C₀-C₆alkyl-C(O)R³, C₀-C₆alkyl-S(O)R³, C₀-C₆alkyl-C(S)R³, and C₀-C₆alkyl-S(O)₂R³;
R¹¹⁰ is:

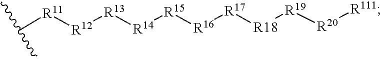

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R¹¹¹ is independently selected from the group consisting of hydrogen, alkyl, cycloakyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-O—P(O)₂R³, C₀-C₆alkyl-O—P(O)₃—R³, C₀-C₆alkyl-O—P(O)(OR³)₂, and C₀-C₆alkylN₃, each of which is optionally substituted with 1, 2, 3, or 4 substituents independently selected at each occurrence from R¹⁰³;
R¹⁰³ is selected at each instance from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅ wherein the optional substituent is selected such that a stable compound results;
and wherein all other variables are as defined herein.

In certain embodiments one of R^{11B}, R^{11C}, R⁵⁵, and R^{66B} is R¹¹⁰. In other embodiments none of R^{11B}, R^{11C}, R⁵⁵, and R^{66B} is R¹¹⁰.

In certain aspects of the invention, an ASGPR binding ligand selected from the following formulas can be used independently from a heterobifunctional molecule:

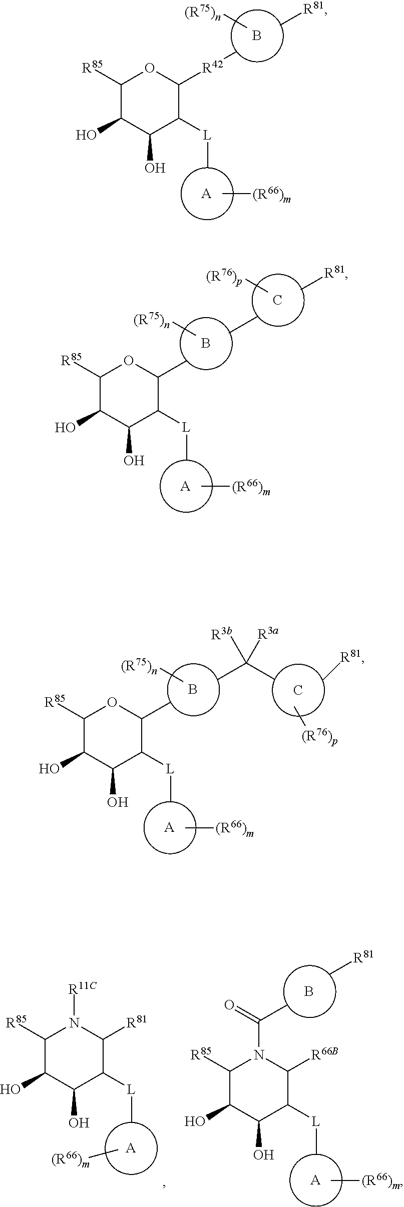

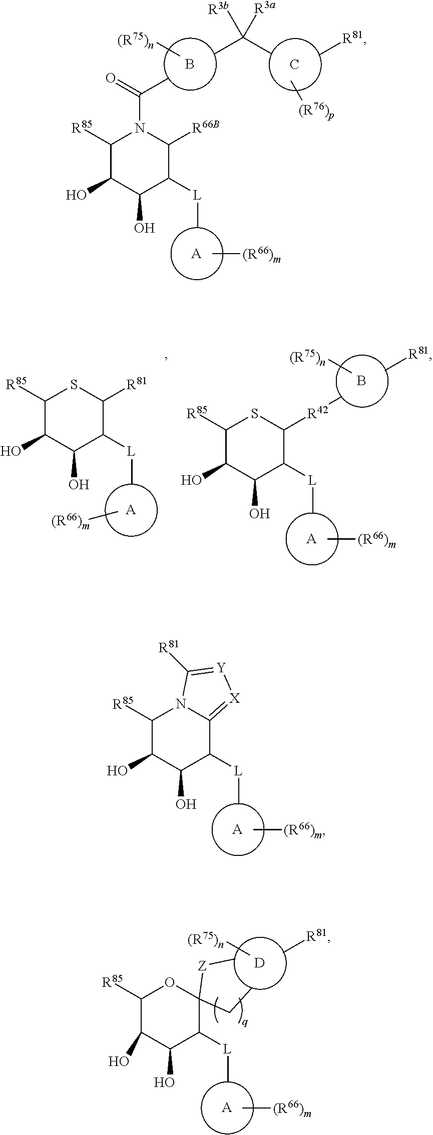

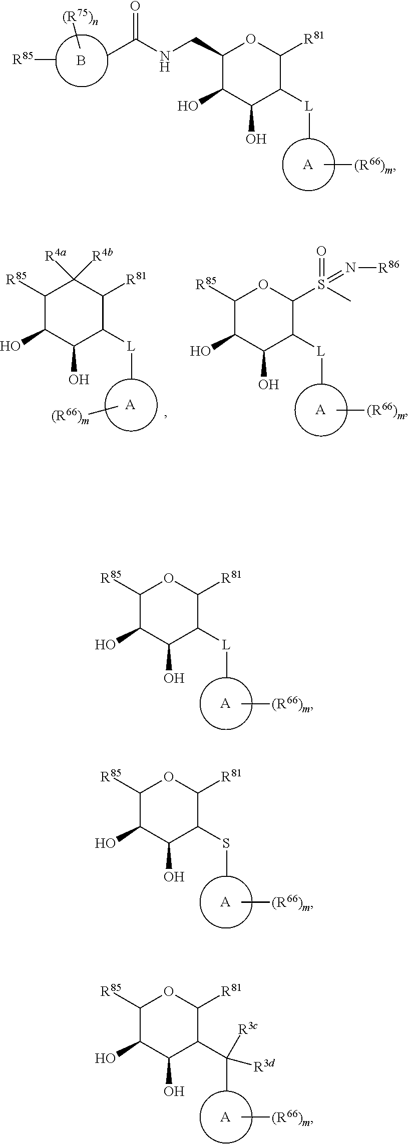

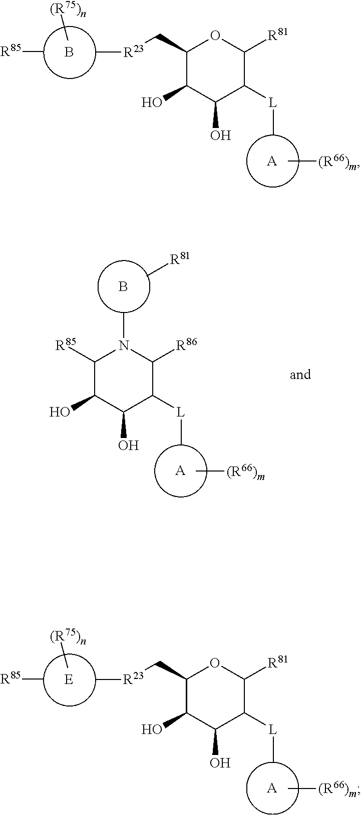

or a salt thereof;
wherein:
R⁸¹, R⁸⁵, and R⁸⁶ are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkylN₃, each of which is optionally substituted with 1, 2, 3, or 4 substituents independently selected at each occurrence from R⁹⁹; and
all other variables are as described herein.

### Additional Embodiments

In certain aspects an extracellular protein degrading compound of Formula I, Formula II, or Formula III is provided:

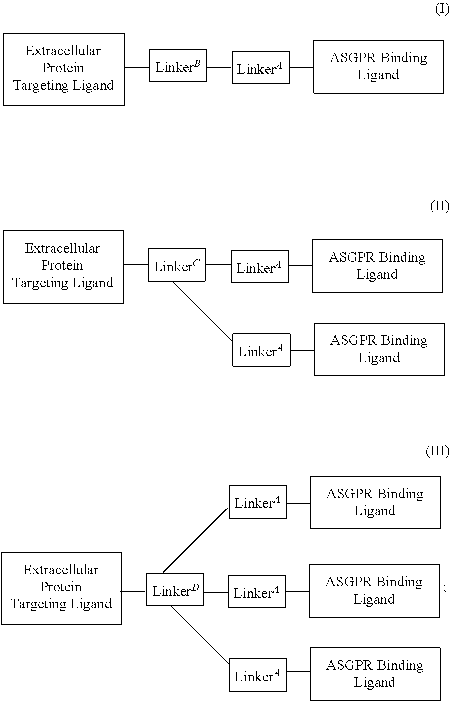

or a pharmaceutically acceptable salt thereof;
wherein the ASGPR Binding Ligand is selected from:

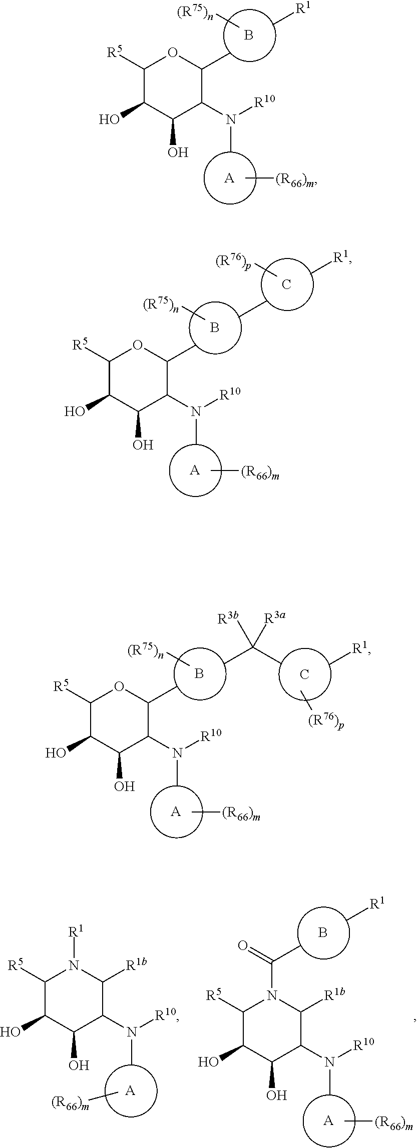

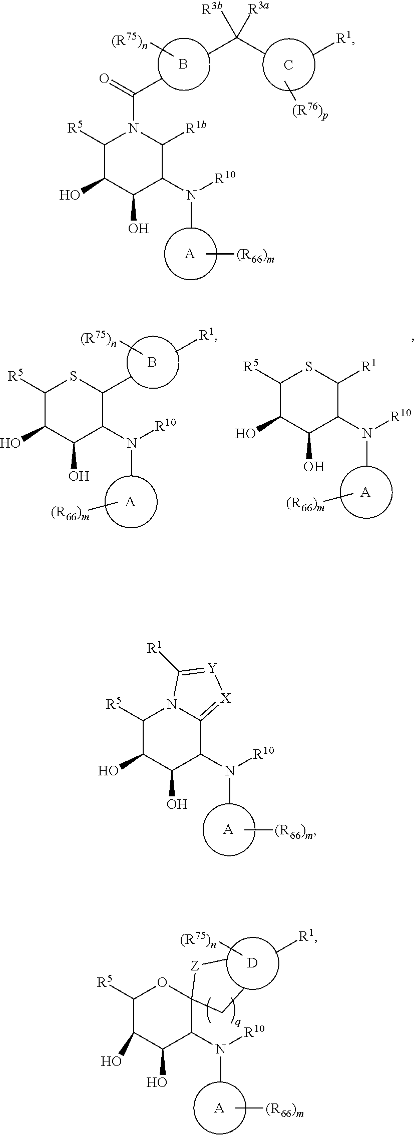

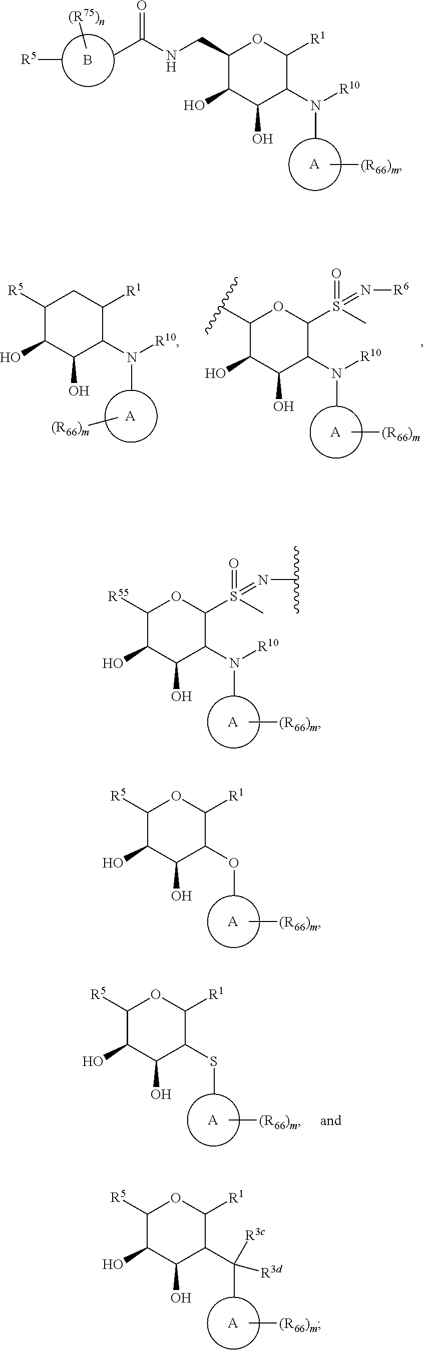

R¹, R^{1b}, and R⁵ are independently selected from hydrogen, C₀-C₆alkyl-cyano, alkyl, alkenyl, alkynyl, haloalkyl, F, Cl, Br, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, and C₀-C₆alkylN₃, each of which is optionally substituted with 1, 2, 3, or 4 substituents independently selected at each occurrence from R⁹⁹;
wherein one of R¹, R^{1b}, and R⁵ is replaced with a bond to Linker^{A};
R³, R^{3a}, R^{3b}, R^{3c}, and R^{3d} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
R⁶⁵, R⁶⁶, and R⁶⁷ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, 3, or 4 substituents independently selected at each occurrence from R¹⁰⁰;
R⁶⁸, R⁶⁹, and R⁷⁰ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R¹⁰¹;
R⁷⁵, R⁷⁶, R⁷⁷, R⁷⁸, and R⁷⁹ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R¹⁰²;
n, m, and p are independently 1, 2, 3, or 4, as allowed by valence;
q is 1, 2, or 3;
A is heteroaryl;
B is aryl, heterocycle, cycloalkyl, or heteroaryl;
C is aryl, heterocycle, cycloalkyl, bicycle, or heteroaryl;
D is aryl or heteroaryl;
X is CH, CR⁷⁵, or N;
Y is CH, CR⁷⁵, or N;
Z is selected from —O—, —NR⁶—, —S—, —S(O)—, —S(O)₂—, and —CR^{3a}R^{3b}—;
R⁹⁹, R¹⁰⁰, R¹⁰¹, R¹⁰² and R¹⁰³ are independently selected at each instance from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅ wherein the optional substituent is selected such that a stable compound results;
Linker^{A} is a bond or a moiety that covalently links Linker^{B}, Linker^{C}, or Linker^{D} to the ASGPR Binding Ligand;
Linker^{B} is a bond or a moiety that covalently links Linker^{A} to an Extracellular Protein Targeting Ligand;
Linker^{C} is a chemical group that links each Linker^{A} to the Extracellular Protein Targeting Ligand;
Linker^{D} is a chemical group that links each Linker^{A} to the Extracellular Protein Targeting Ligand; and
Extracellular Protein Targeting Ligand is a Ligand that binds to an extracellular protein.

In certain embodiments the ASPGR Binding Ligand is a compound selected from:

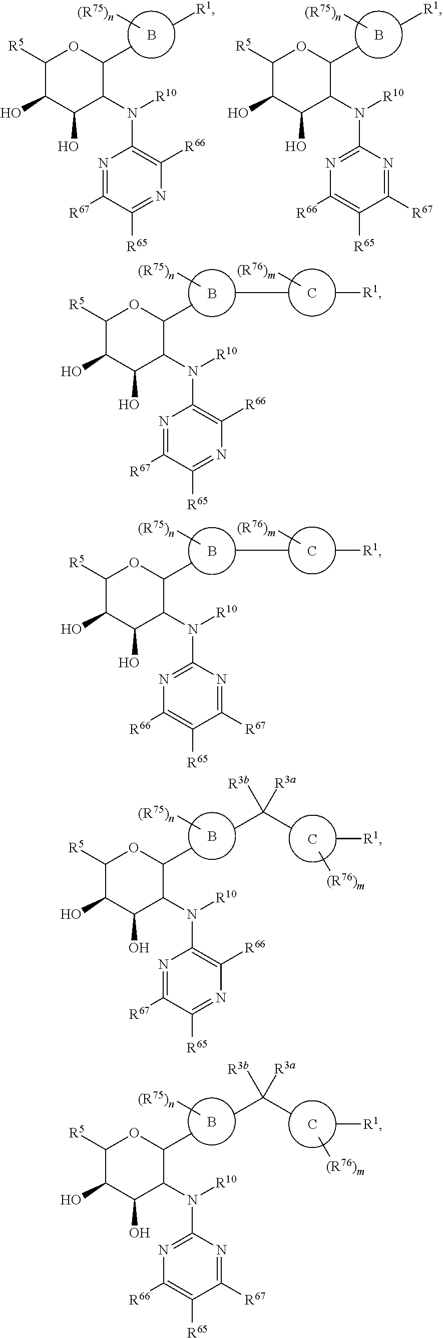

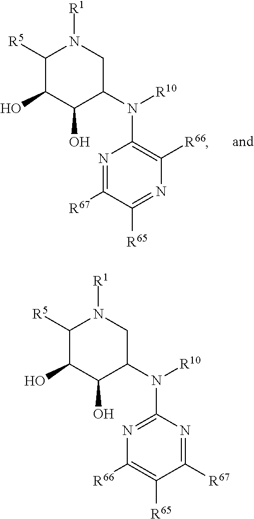

or a pharmaceutically acceptable salt thereof.

In certain embodiments the ASPGR Binding Ligand is a compound selected from:

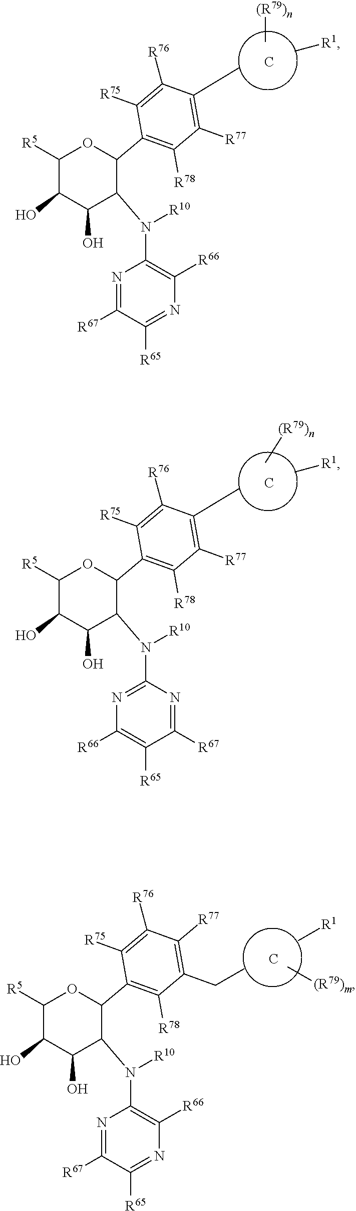

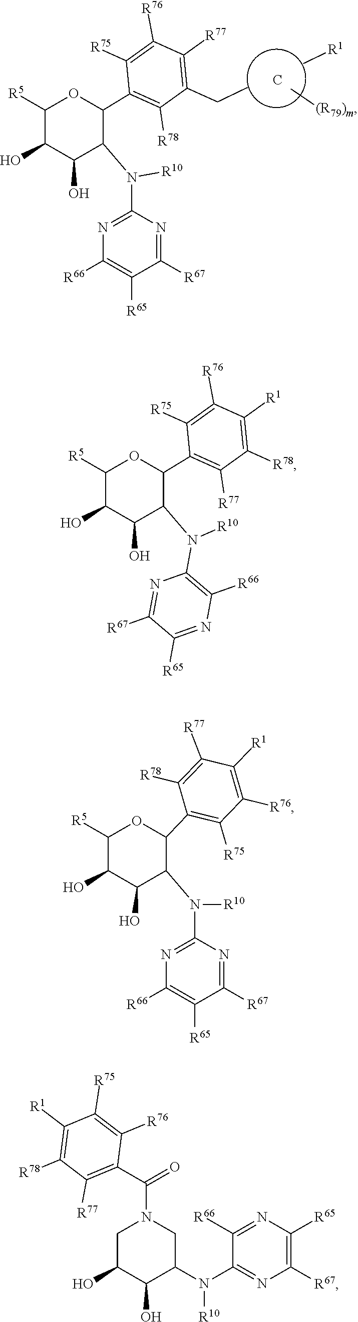

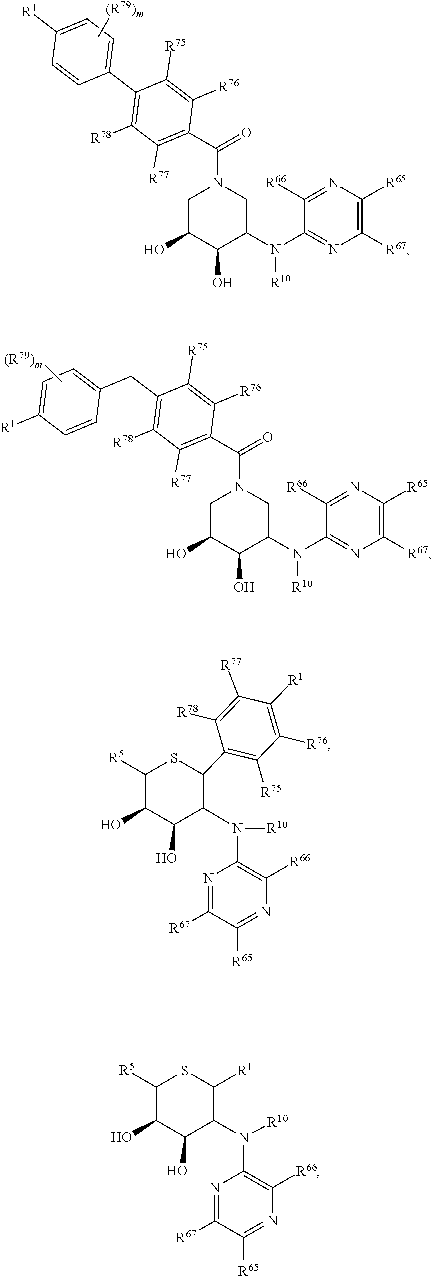

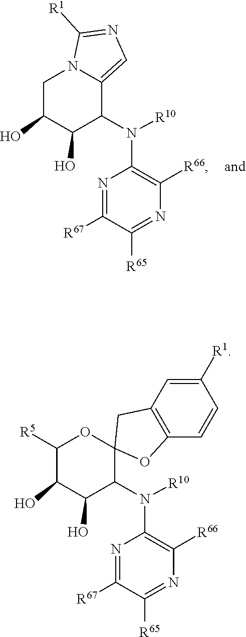

In certain embodiments ASGPR Binding Ligand is a compound selected from:

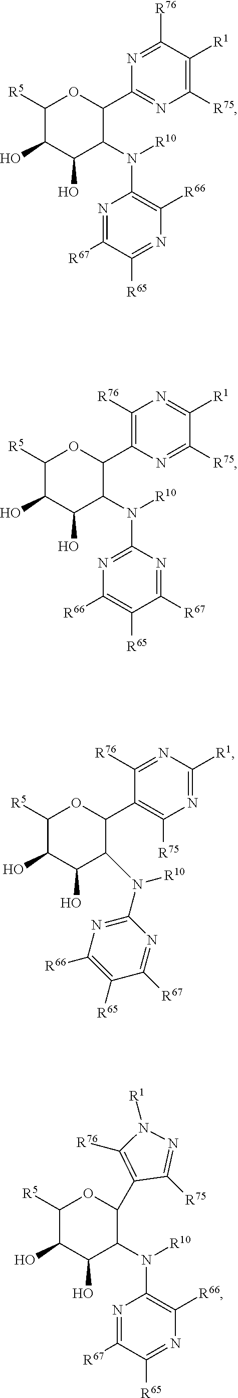

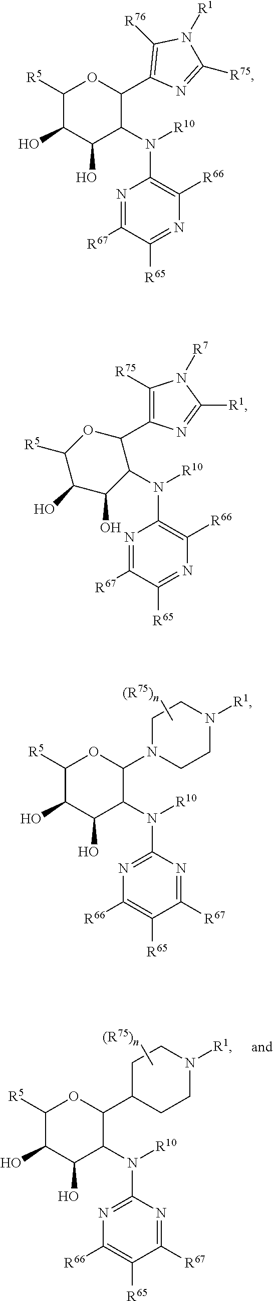

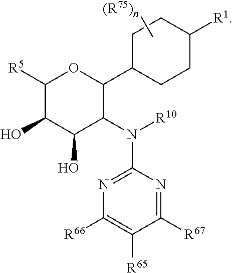

In certain embodiments the ASGPR Binding Ligand is a compound selected from:

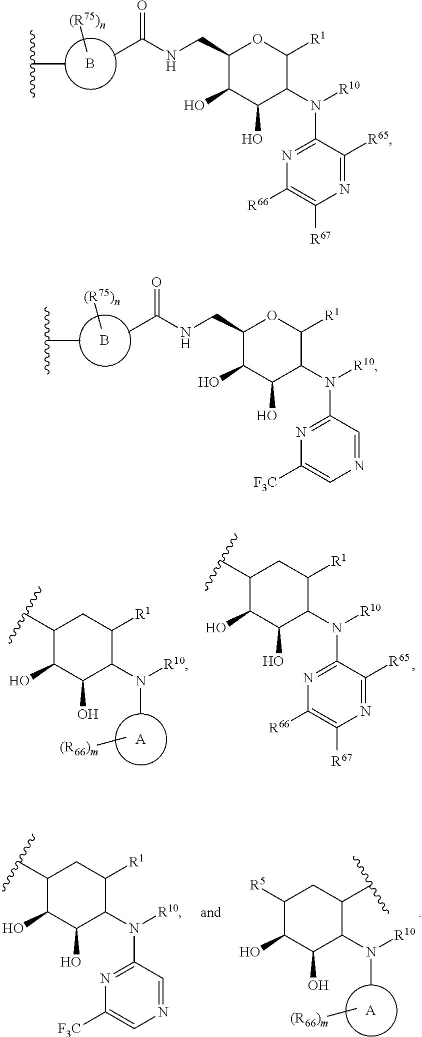

In certain embodiments the ASGPR Binding Ligand is a compound selected from:

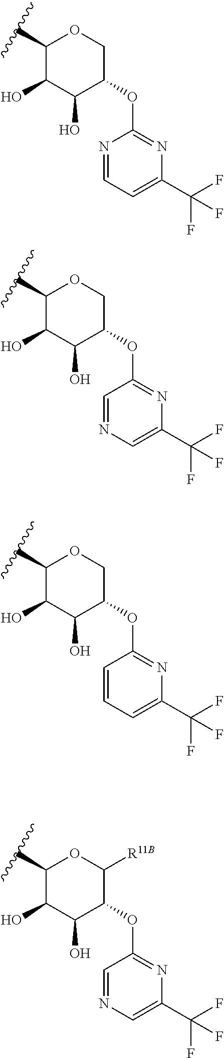

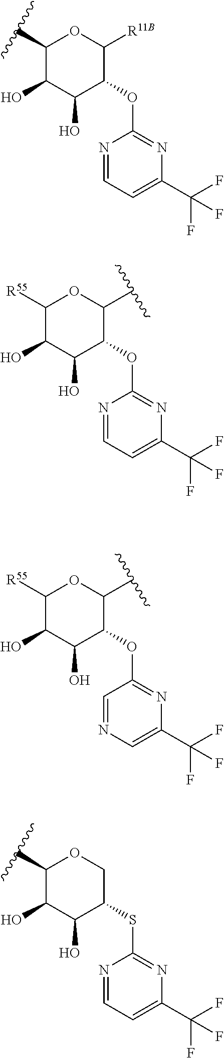

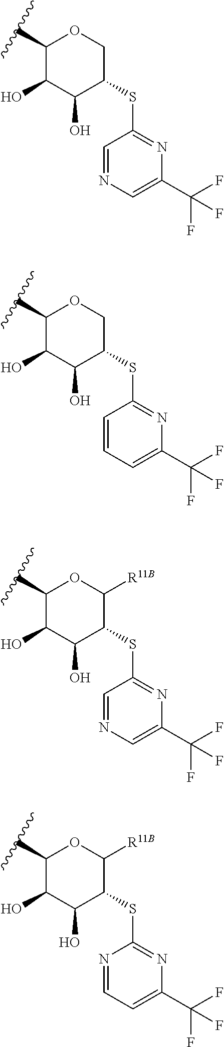

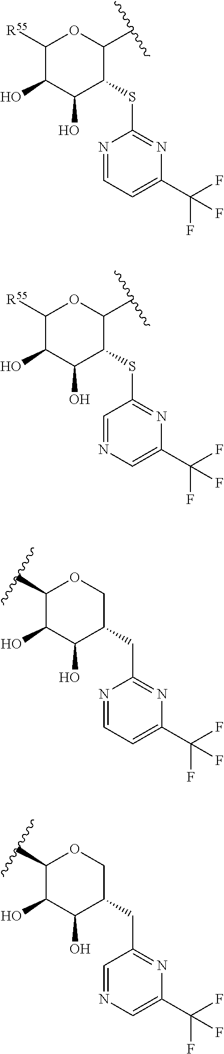

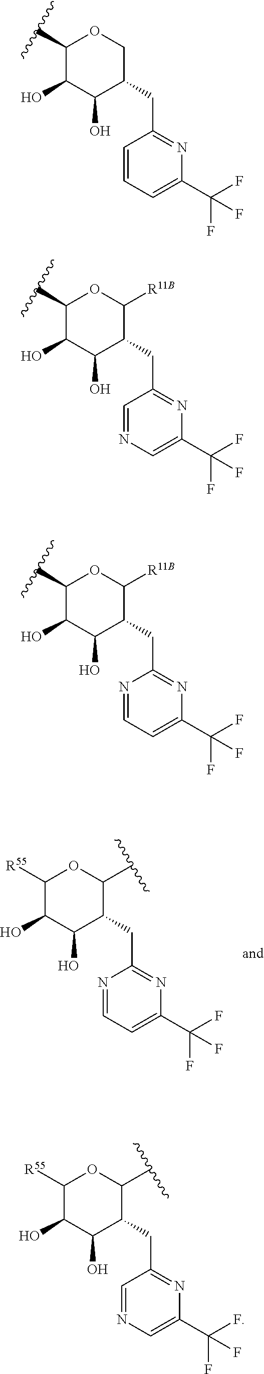

In certain embodiments the ASGPR Binding Ligand is a compound selected from:

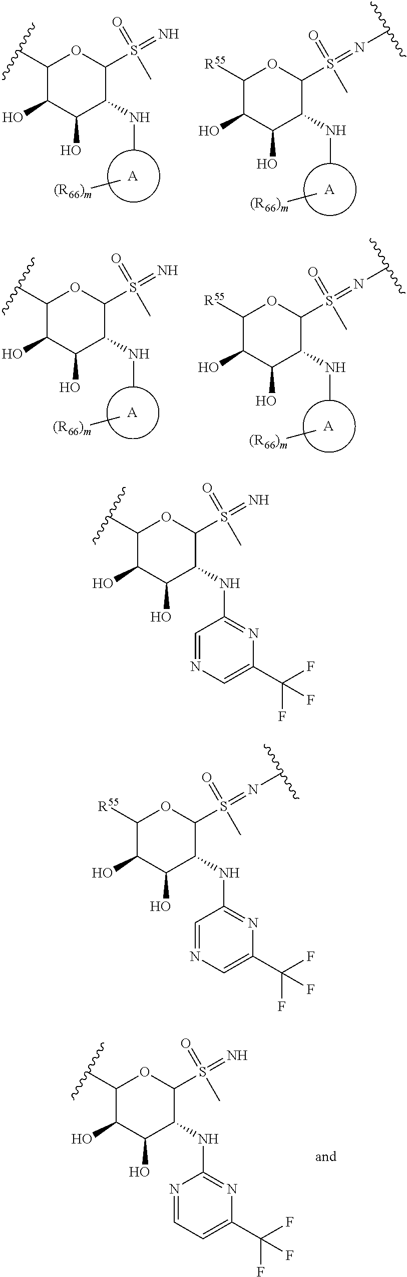

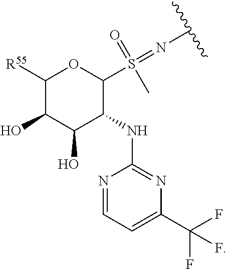

In some aspects of the invention, the Extracellular Protein Targeting Ligand targets an immunoglobulin, for example IgG, IgA or IgE.

In certain aspects of the invention, an ASGPR ligand selected from the following formulas can be used independently from a heterobifunctional molecule:

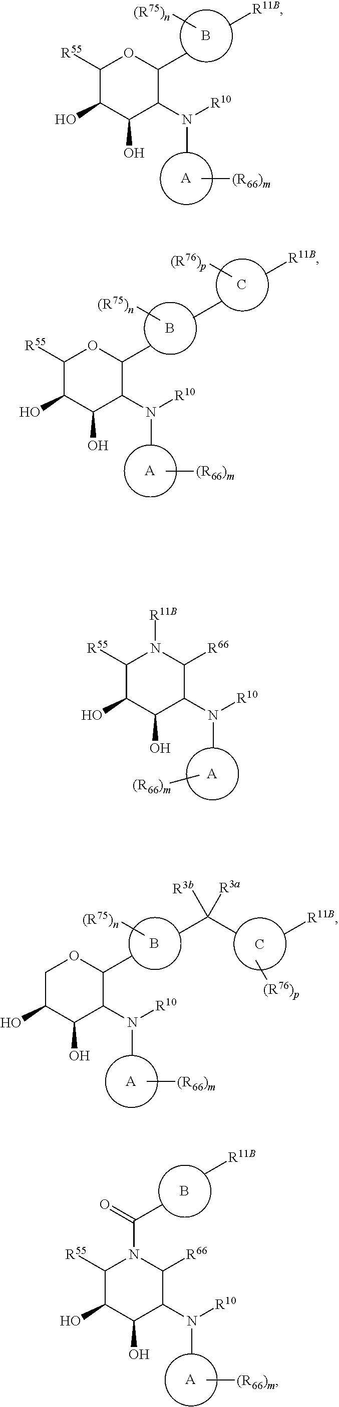

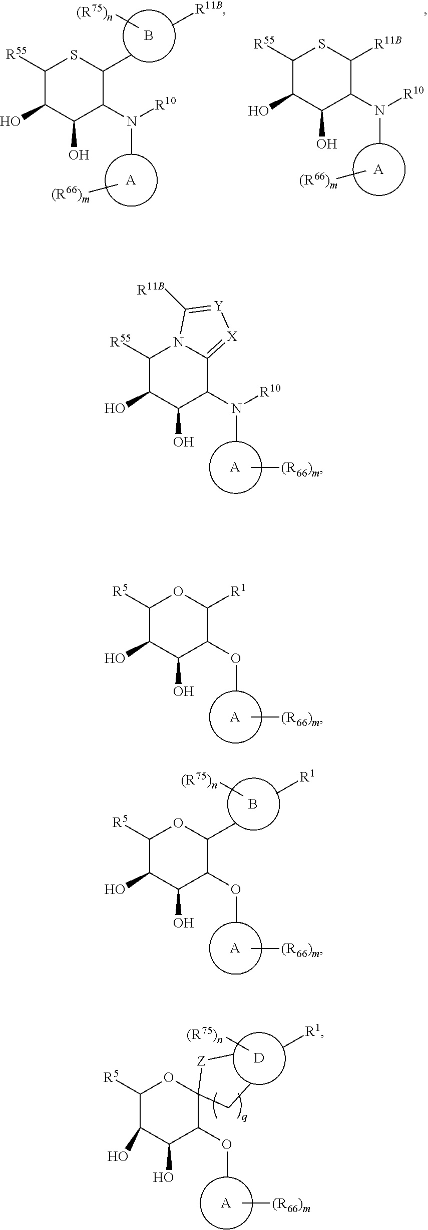

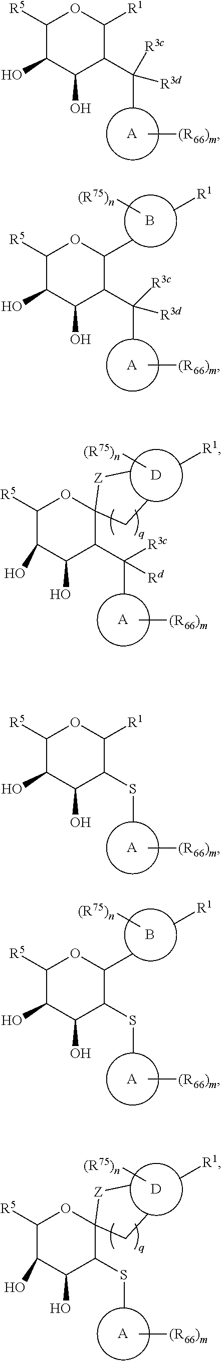

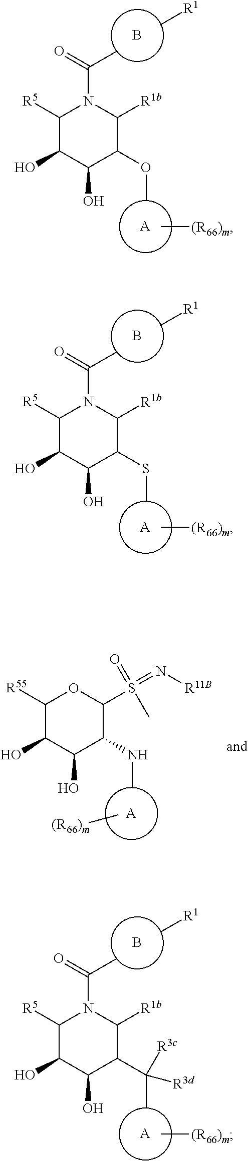

or a ASGPR ligand selected from the following formulas

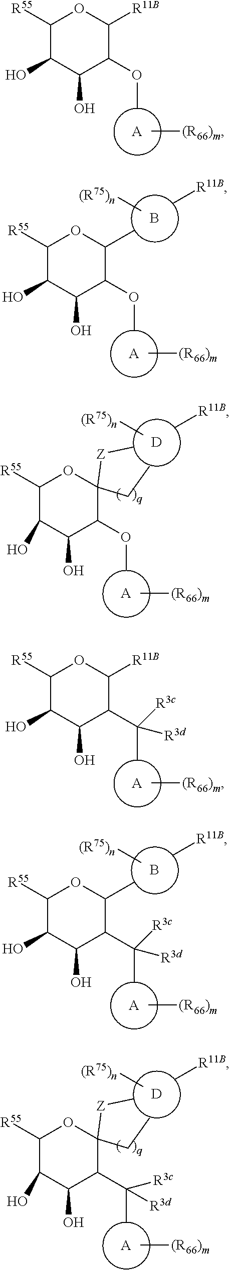

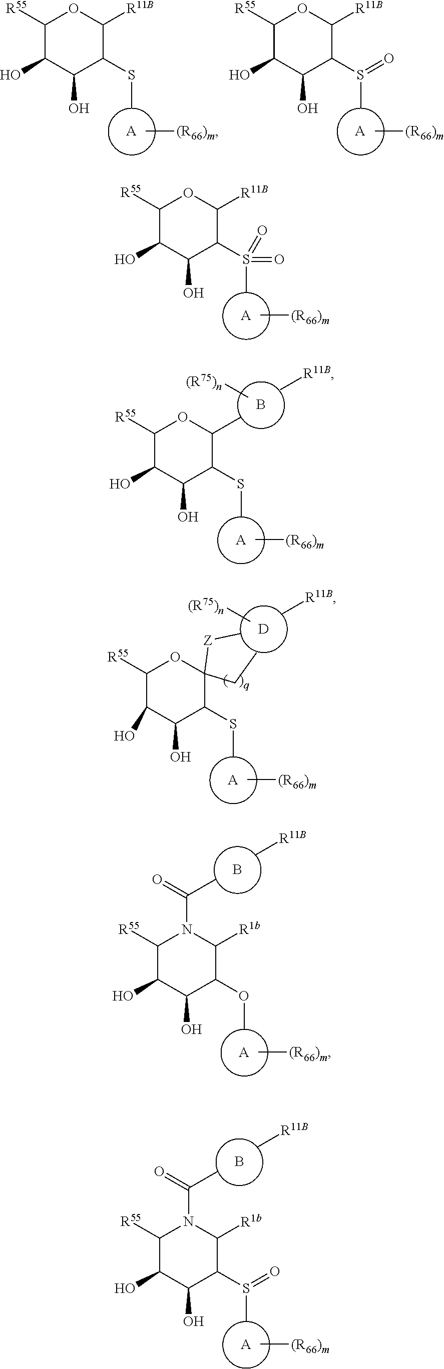

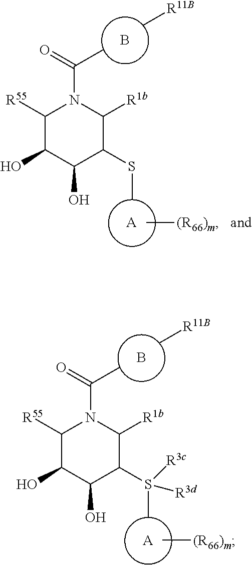

or a salt thereof;
wherein:
R^{11B}, R⁵⁵, and R^{66B} are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkylN₃, and R¹¹⁰ each of which is optionally substituted with 1, 2, 3, or 4 substituents independently selected at each occurrence from R⁹⁹;
R¹¹⁰ is:

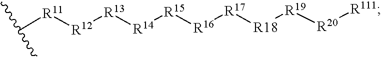

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R¹¹¹ is independently selected from the group consisting of hydrogen, alkyl, cycloakyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-O—P(O)₂R³, C₀-C₆alkyl-O—P(O)₃—R³, C₀-C₆alkyl-O—P(O)(OR³)₂, and C₀-C₆alkylN₃, each of which is optionally substituted with 1, 2, 3, or 4 substituents independently selected at each occurrence from R¹⁰³;
and wherein all other variables are as defined herein.

In certain embodiments one of R^{11B}, R⁵⁵, and R^{66B} is R¹¹⁰. In other embodiments none of R^{11B}, R⁵⁵, and R^{66B} are R¹¹⁰.

In certain embodiments, R^{11B} is R¹¹¹.

In certain embodiments, R⁵⁵ is R¹¹¹.

In certain embodiments, R^{66B} is R¹¹¹.

In certain embodiments, R¹¹¹ is selected from the group consisting of C₀-C₆alkyl-NR⁶R⁷, C₀-C₆alkyl-C(O)OCH₃, C₀-C₆alkyl-C(O)OH, C₀-C₆alkyl-C(O)H, C₀-C₆alkyl-OR⁶, C₀-C₆alkyl-OH, C₀-C₆alkyl-O(O)CH₂CH₃, haloalkyl, F, Cl, Br, I, and C₀-C₆alkylN₃.

In certain embodiments, R¹¹¹ is —NH₂.

In certain embodiments, R¹¹¹ is —NHR⁷.

In certain embodiments, R¹¹¹ is —C(O)H.

In certain embodiments, R¹¹¹ is —C(O)OH.

In certain embodiments, R¹¹¹ is —C(O)OCH₃.

In certain embodiments, R¹¹¹ is —N₃.

In certain embodiments, R¹¹¹ is —(CH₂)₂, —N₃.

In certain embodiments, R¹¹¹ is alkynyl.

In certain embodiments, R¹¹¹ is aryl.

In certain embodiments, R¹¹¹ is heteroaryl.

In certain embodiments, R¹¹¹ is heterocycle.

In certain embodiments, R¹¹¹ is aryl substituted with 1, 2, 3, or 4 substituents selected from R¹⁰³.

In certain embodiments, R¹¹¹ is heteroaryl substituted with 1, 2, 3, or 4 substituents selected from R¹⁰³.

In certain embodiments, R¹¹¹ is heterocycle substituted with 1, 2, 3, or 4 substituents selected from R¹⁰³.

In certain embodiments, R¹¹¹ is haloalkoxy.

In alternative embodiments, R¹¹¹ is a sulfonyl group. Nonlimiting examples of sulfonyl groups include mesyl, tosyl, brosyl, nosyl and triflyl group.

In certain embodiments, R¹¹¹ is a protecting group. Nonlimiting examples of protecting groups include acetyl (Ac), benzoyl (Bz), benzyl (Bn), methoxyethoxymethyl ether (MEM), dimethoxytrityl, [bis-(4-methoxyphenyl)phenylmethyl](DMT), methoxymethyl ether (MOM), methoxytrityl [(4-methoxyphenyl)diphenylmethyl](MMT), p-methoxybenzyl ether (PMB), p-methoxyphenyl ether (PMP), methylthiomethyl ether, pivaloyl (Piv), tert-butyl ethers (tBu), tetrahydropyranyl (THP), tetrahydrofuranyl (THF), trityl (triphenylmethyl, Tr), silyl ether (including but not limited to trimethylsilyl (TMS), tert-butyldimethylsilyl (TBDMS or TBS), tri-iso-propylsilyloxymethyl (TOM), and triisopropylsilyl (TIPS) ethers), methyl ethers, ethoxyethyl ethers (EE), carbobenzyloxy (Cbz), p-methoxybenzyloxycarbonyl (Moz or MeOZ), tert-butyloxycarbonyl (BOC) group, 9-fluorenylmethyloxycarbonyl (Fmoc), 3,4-dimethoxybenzyl (DMPM), tosyl (Ts), Troc (trichloroethyl chloroformate), and nosyl (Ns).

In certain aspects of the invention, an ASGPR binding ligand selected from the following formulas can be used independently from a heterobifunctional molecule:

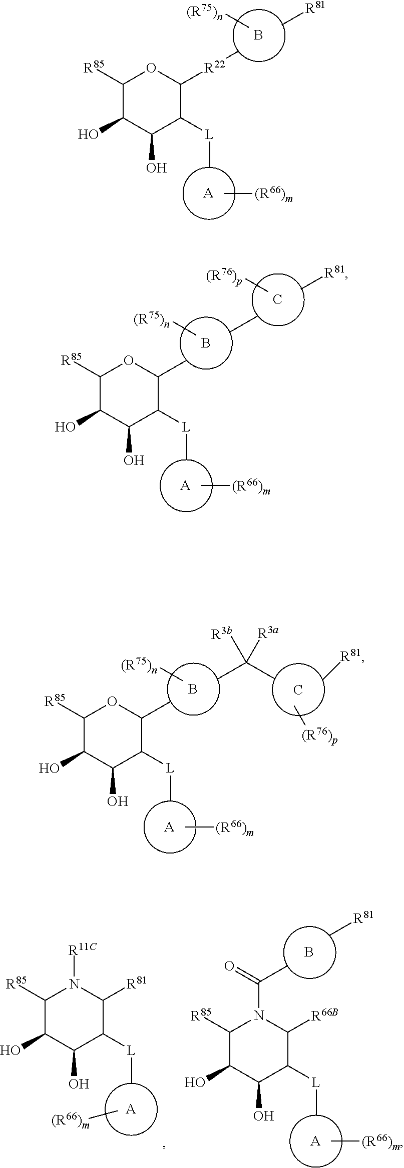

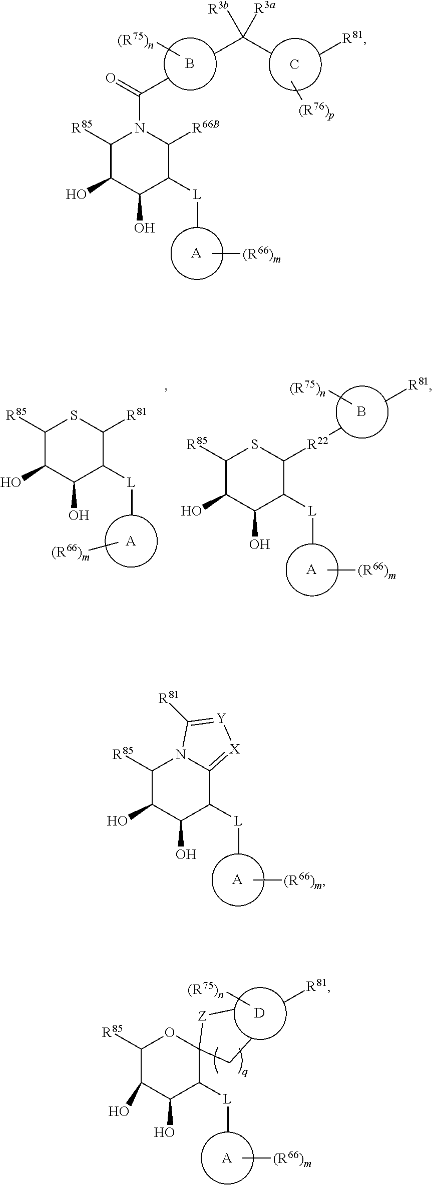

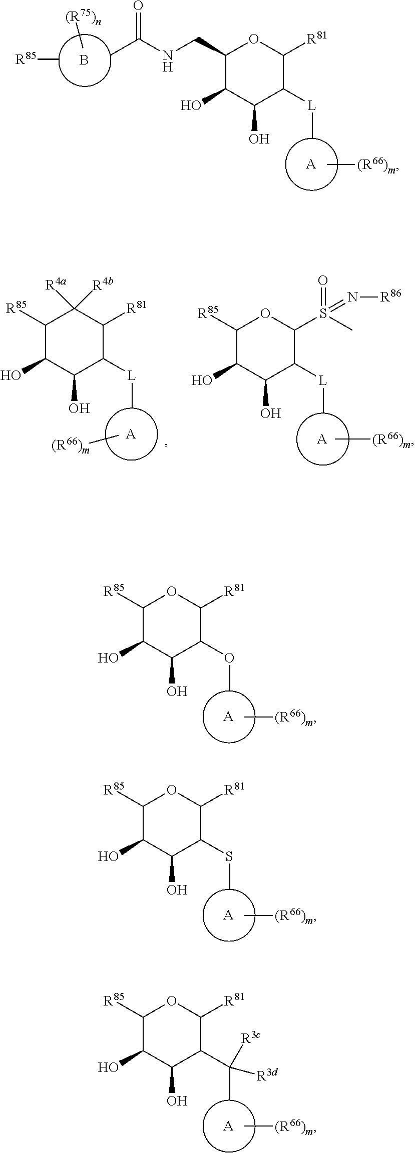

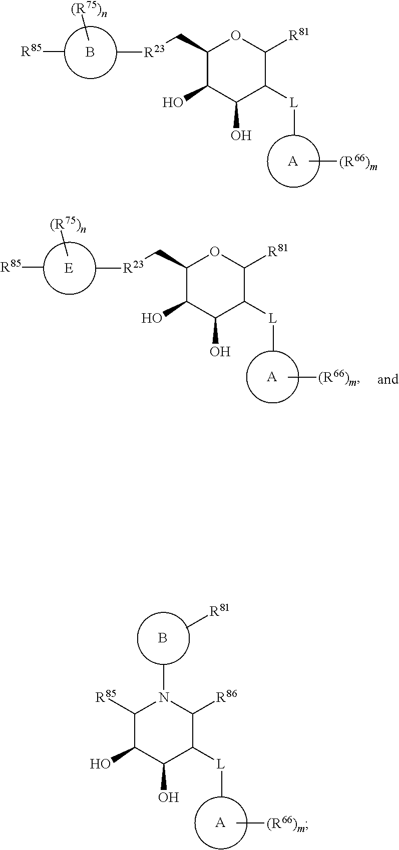

or a salt thereof;
wherein:
R⁸¹, R⁸⁵, and R⁸⁶ are independently selected from hydrogen, C₀-C₆alkyl-cyano, alkyl, alkenyl, C₀-C₆alkyl-alkynyl, haloalkyl, F, Cl, Br, I, C₀-C₆alkyl·aryl, C₀-C₆alkyl·heteroaryl, C₀-C₆alkyl·heteroarylalkyl, C₀-C₆alkyl·heterocycle, C₀-C₆alkyl·heterocycloalkyl, C₀-C₆alkyl·haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkylN₃, each of which is optionally substituted with 1, 2, 3, or 4 substituents independently selected at each occurrence from R⁹⁹; and
all other variables are as described herein.
   1. In certain embodiments, R¹¹ is hydrogen.
   2. In certain embodiments, R⁸¹ is C₀-C₆alkyl-alkynyl.
   3. In certain embodiments, R¹¹ is aryl.
   4. In certain embodiments, R¹¹ is phenyl.
   5. In certain embodiments, R¹¹ is naphthyl.
   6. In certain embodiments, R¹¹ is heteroaryl.
   7. In certain embodiments, R¹¹ is heteroaryl.
   8. In certain embodiments, R⁸¹ is C₀-C₆alkyl·heteroaryl.
   9. In certain embodiments, R⁸¹ is C₀-C₆alkyl·triazole.
   10. In certain embodiments, R¹¹ is pyrazine.
   11. In certain embodiments, R¹¹ is pyridazine.
   12. In certain embodiments, R¹¹ is pyrimidine.
   13. In certain embodiments, R¹¹ is quinoline.
   14. In certain embodiments, R¹¹ is haloalkyl.
   15. In certain embodiments, R¹¹ is bromo.
   16. In certain embodiments, R¹¹ is chloro.
   17. In certain embodiments, R⁸¹ is C₀-C₆alkylN₃.
   18. In certain embodiments, R⁸¹ is C₀-C₆alkyl-OR⁶.
   19. In certain embodiments, R⁸¹ is C₀-C₆alkyl-OH.
   20. In certain embodiments, R⁸¹ is C₀-C₆alkyl-Oalkyl.
   21. In certain embodiments, R⁸¹ is C₀-C₆alkyl-Oalkenyl.
   22. In certain embodiments, R81 is —OAllyl.
   23. In certain embodiments, R⁸¹ is C₀-C₆alkyl-SR⁶.
   24. In certain embodiments, R⁸¹ is C₀-C₆alkyl-NR⁶R⁷.
   25. In certain embodiments, R⁸¹ is C₀-C₆alkyl-NHC(O)OtBu.
   26. In certain embodiments, R⁸¹ is C₀-C₆alkyl-NHC(O)OBn.
   27. In certain embodiments, R⁸¹ is C₀-C₆alkyl-C(O)R³.
   28. In certain embodiments, R⁸¹ is C₀-C₆alkyl-C(O)OH.
   29. In certain embodiments, R⁸¹ is C₀-C₆alkyl-S(O)R³.
   30. In certain embodiments, R⁸¹ is C₀-C₆alkyl-C(S)R³.
   31. In certain embodiments, R¹¹ is C₀-C₆alkyl-S(O)₂R³.
   32. Any one of embodiments 1-31, wherein R⁸¹ is optionally substituted.
   33. Embodiment 32, wherein R⁸¹ has one substituent selected from R⁹⁹.
   34. Embodiment 32, wherein R⁸¹ has two substituents independently selected at each instance from R⁹⁹.
   35. Embodiment 32, wherein R⁸¹ has three substituents independently selected at each instance selected from R⁹⁹.
   36. Embodiment 32, wherein R⁸¹ has four substituents independently selected at each instance selected from R⁹⁹.
1. An ASGPR-binding extracellular protein degrader compound of the formula:

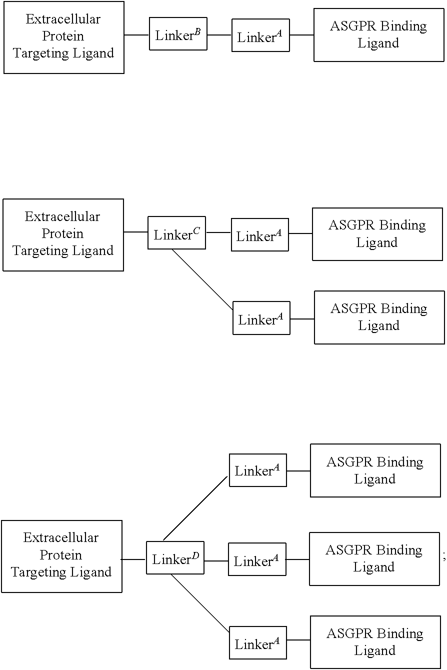

or a pharmaceutically acceptable salt thereof;
   wherein the ASGPR Binding Ligand is selected from:

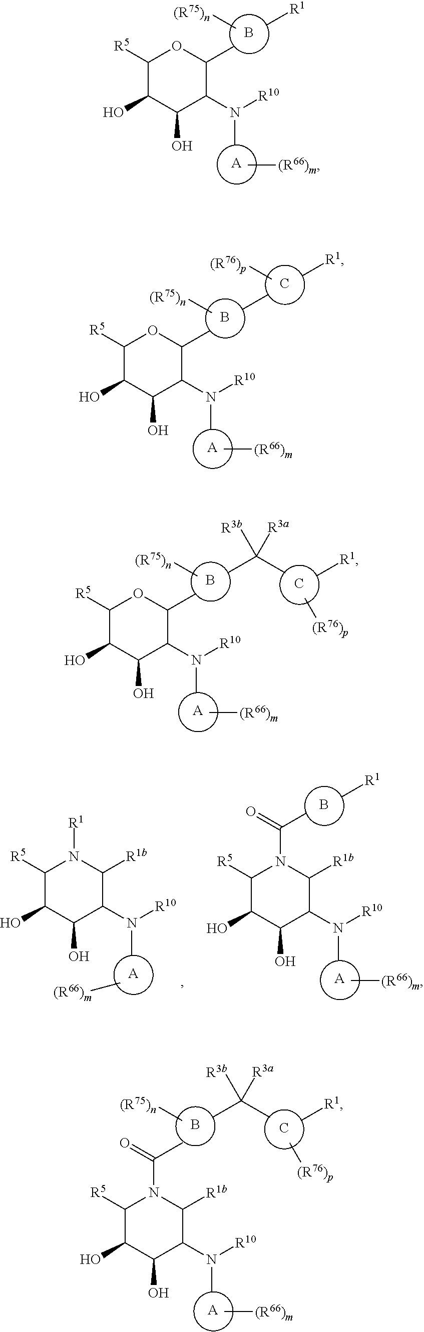

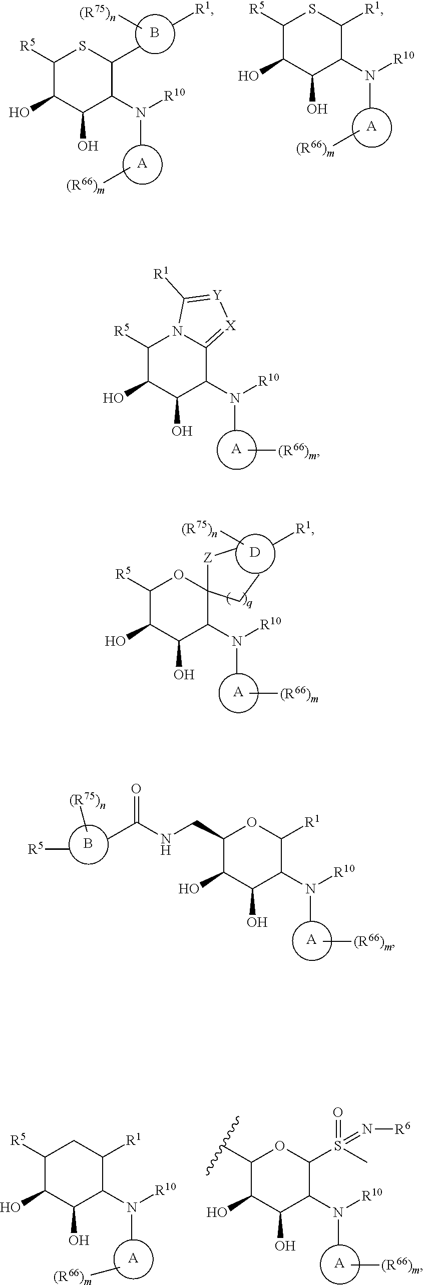

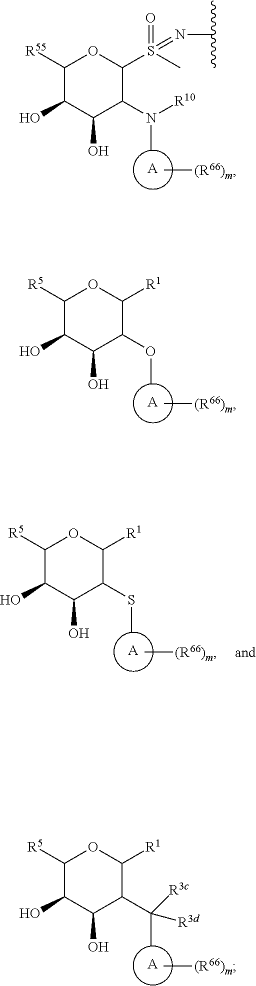

R¹ and R⁵ are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, and C₀-C₆alkylN₃, each of which is optionally substituted with 1, 2, 3, or 4 substituents independently selected at each occurrence from R⁹⁹;
R³, R^{3a}, and R^{3b} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
R⁶⁵, R⁶⁶, and R⁶⁷ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, 3, or 4 substituents independently selected at each occurrence from R⁹⁹;
R⁶⁸, R⁶⁹, and R⁷⁰ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R⁹⁹;
R⁷⁵, R⁷⁶, R⁷⁷, R⁷⁸, and R⁷⁹ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R⁹⁹;
n, m, and p are independently 1, 2, 3, or 4, as allowed by valence;
q is 1, 2, or 3;
A is heteroaryl;
B is aryl, heterocycle, cycloalkyl, or heteroaryl;
C is aryl, heterocycle, cycloalkyl, bicycle, or heteroaryl;
D is aryl or heteroaryl;
X is CH, CR⁷⁵, or N;
Y is CH, CR⁷⁵, or N;
Z is selected from —O—, —NR⁶—, —S—, —S(O)—, —S(O)₂—, and —CR^{3a}R^{3b}—; and
R⁹⁹, R¹⁰⁰, R¹⁰¹, R¹⁰² and R¹⁰³ are independently selected at each instance from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅ wherein the optional substituent is selected such that a stable compound results;
Linker^{A} is a bond or a moiety that covalently links Linker^{B}, Linker^{C}, or Linker^{D} to the ASGPR Binding Ligand;
Linker^{B} is a bond or a moiety that covalently links Linker^{A} to an Extracellular Protein Targeting Ligand;
Linker^{C} is a chemical group that links each Linker^{A} to the Extracellular Protein Targeting Ligand;
Linker^{D} is a chemical group that links each Linker^{A} to the Extracellular Protein Targeting Ligand; and
Extracellular Protein Targeting Ligand is a Ligand that binds to an extracellular protein.
2. The compound of embodiment 1, wherein the ASPGR Binding Ligand is a compound selected from:

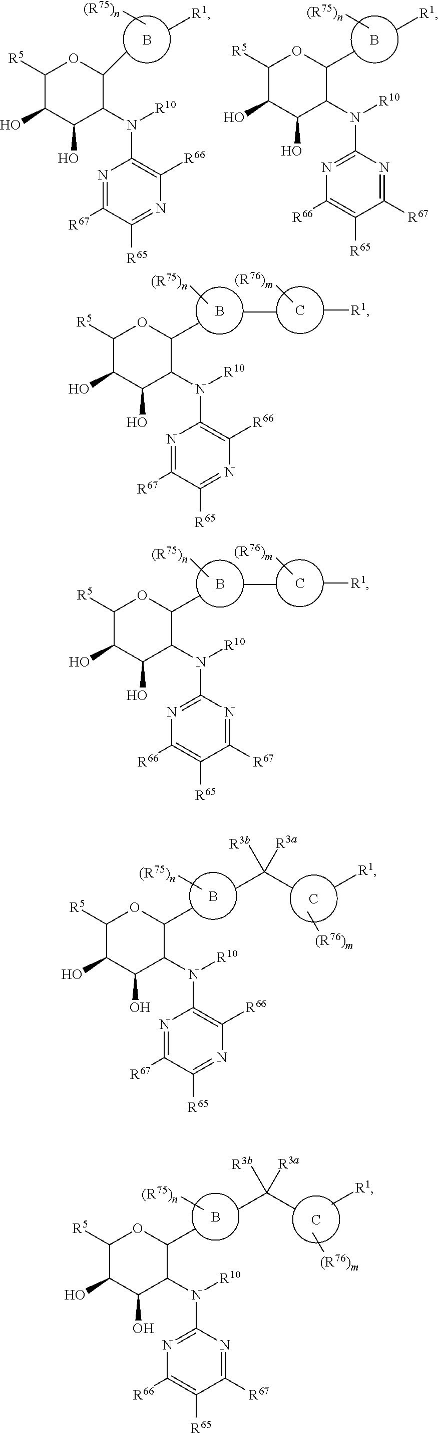

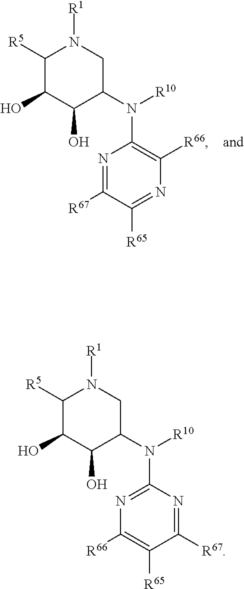

3. The compound of embodiment 1, wherein the ASPGR Binding Ligand is a compound selected from:

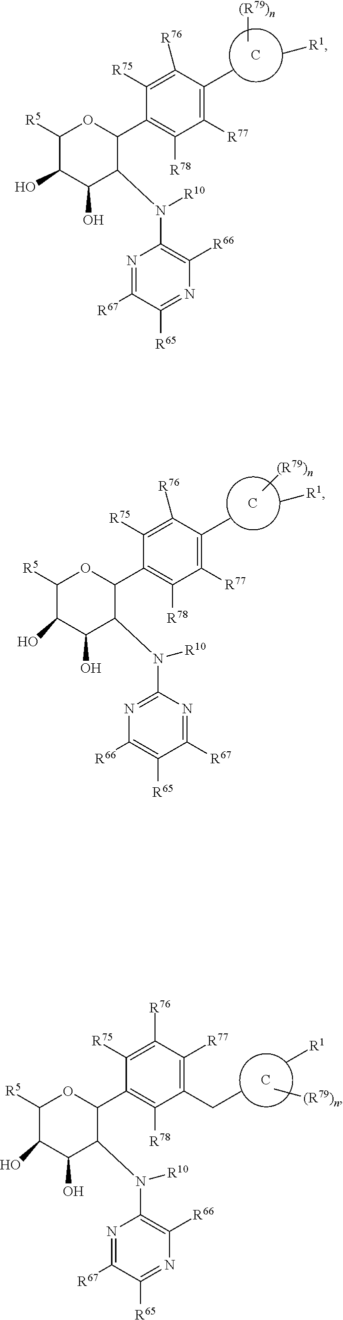

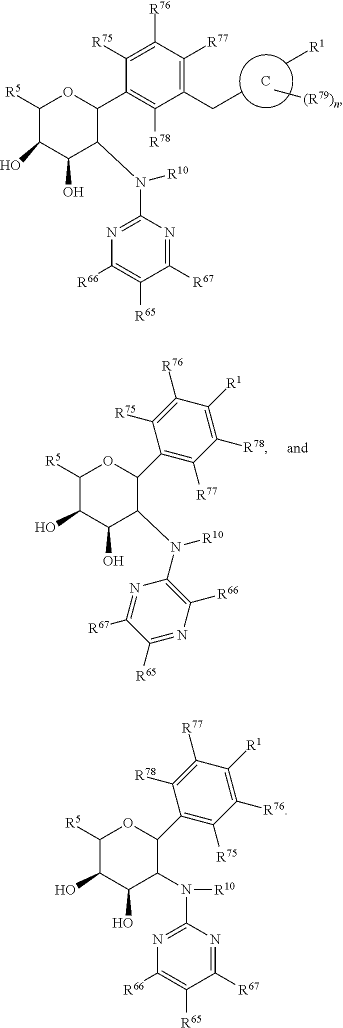

4. The compound of embodiment 1, wherein the ASPGR Binding Ligand is a compound selected from:

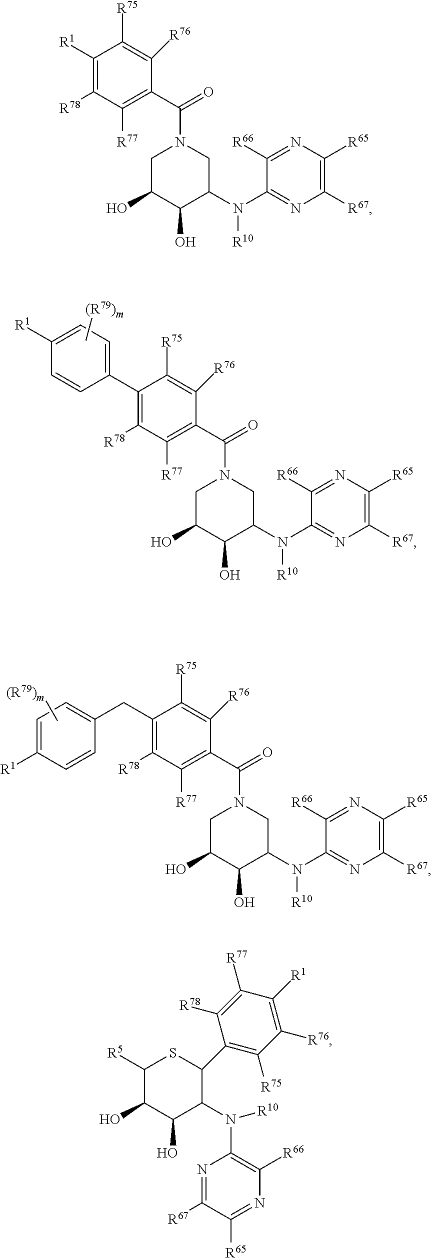

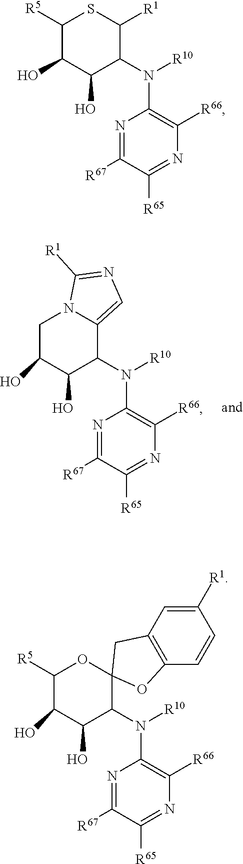

5. The compound of embodiment 1, wherein the ASGPR Binding Ligand is selected from:

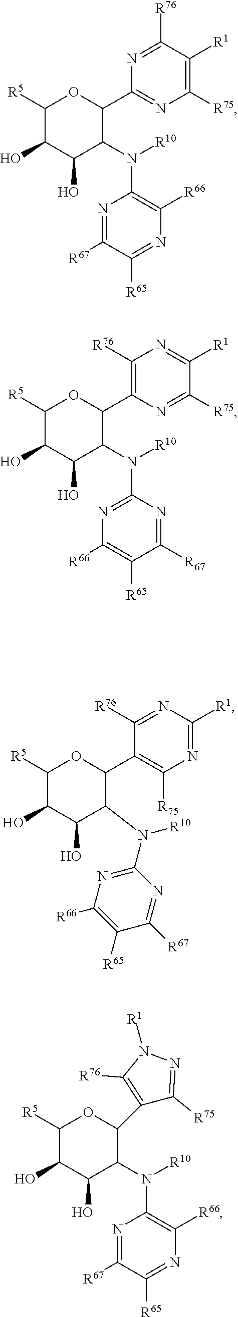

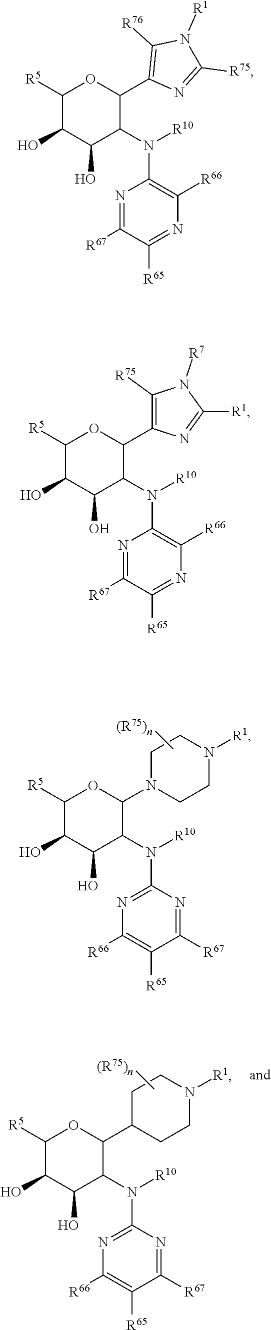

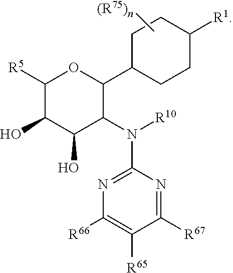

6. The compound of any one of embodiments 1-5, wherein the Extracellular Protein Targeting Ligand binds IgG.
7. The compound of embodiment 6, wherein the IgG targeting Extracellular Protein Targeting Ligand is Fc-BP-2.
8. The compound of embodiment 6, wherein the IgG targeting Extracellular Protein Targeting Ligand is Fc-III.
9. A method of treating a disorder mediated by an Extracellular Protein, comprising administering an effective amount of compound of any one of embodiments 1-8 that includes an Extracellular Targeting Protein Ligand which binds to the Extracellular Protein, or a pharmaceutically acceptable salt or pharmaceutical composition thereof, to a patient in need thereof.
10. The method of embodiment 9, wherein the Extracellular Protein is IgG and the disorder is selected from antiphospholipid Ab syndrome, Behcet syndrome, Hashimoto thyroiditis, MGUS, necrobiotic xanthogranuloma, rheumatoid arthritis, cancer, for example multiple myeloma or peripheral multiple myeloma, paraproteinemia, chronic urticaria, scleroderma, scleromyxedema, thrombocytopenia for example heparin-induced thrombocytopenia, cryoglobulinema, granulomatosis with polyanglititis, for example ANCA associated vasculitis, idiopathic thrombocytopenic purpura, thrombocytopenia, IgG4-RD, paroxysmal nocturnal hemoglobinuria (PNH), warm autoimmune hemolytic anemia, rhabdomyolysis, lupus nephritis, acute disseminated encephalomyelitis, Guillaine-Barre syndrome, chronic inflammatory demyelinating polyneuropathy, Miller Fisher syndrome, neuromyelitis optica spectrum disorder, opsoclonus-myoclonus syndrome, pediatric autoimmune neuropsychiatric disorder associated with streptococcal infection (PANDAS), peripheral neuropathy, transverse myelitis, fibrosis, IPF/fibrosis, and transplantation rejection.
11. The method of embodiment 9, wherein the Extracellular Protein is IgG4 and the disorder is selected from type 1 autoimmune pancreatitis, interstitial nephritis, Riedel's thyroiditis, storiform fibrosis, Mikulicz's disease, Küttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementemic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenia gravis, ankylosing spondylitis, primary Sjögren's syndrome, psoriatic arthritis, systemic lupus erythematosus (SLE), sclerosing cholangitis, IgG monoclonal gammopathy, monoclonal gammopathy of undetermined significance (MGUS), melanoma, bullous pemphigoid, Goodpasture disease, encephalitis, thrombotic thrombocytopenic purpura, chronic inflammatory polyneuropathy, limbic encephalitis, neuromyotonia, Morvan syndrome, pemphigus foliaceus, pemphigus vulgaris, REM and non-REM parasomnia, and membranous nephropathy, multiple sclerosis, hyperthyroid Grave's disease, epidermolysis bullosa acquisita, pemphigoid gestationis, anti-p200 pemphigoid, and paraneoplastic pemphigus.
12. The method of embodiment 9, wherein the Extracellular Protein is IgA and the disorder is selected from including IgA nephropathy (also known as Berger's disease), celiac disease, Crohn's disease, Henoch-Schonlein purpura (HSP) (also known as IgA vasculitis), IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), linear IgA bullous dermatosis, rheumatoid arthritis, ulcerative colitis, and primary glomerulonephritis.
13. A compound of formula:

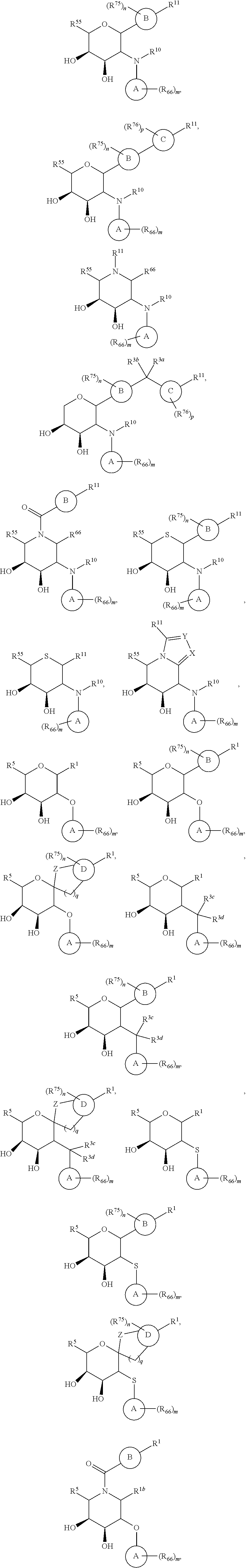

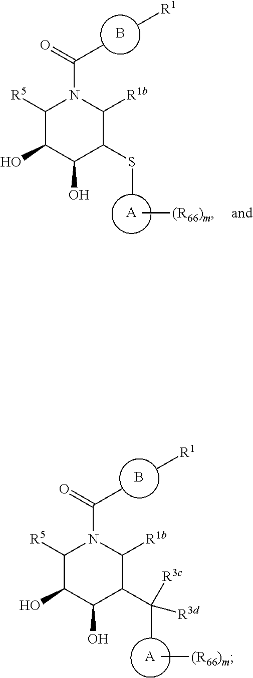

or a salt thereof;
wherein:
R¹¹, R⁵⁵, and R⁶⁶ are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, and C₀-C₆alkylN₃, each of which is optionally substituted with 1, 2, 3, or 4 substituents independently selected at each occurrence from R⁹⁹;
R³, R^{3a}, R^{3b}, R^{3c}, and R^{3d} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
R⁶⁵, R⁶⁶, and R⁶⁷ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, 3, or 4 substituents independently selected at each occurrence from R¹⁰⁰;
R⁶⁸, R⁶⁹, and R⁷⁰ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R¹⁰¹;
R⁷⁵, R⁷⁶, R⁷⁷, R⁷⁸, and R⁷⁹ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R¹⁰²;
n, m, and p are independently 1, 2, 3, or 4, as allowed by valence;
q is 1, 2, or 3;
A is heteroaryl;
B is aryl, heterocycle, cycloalkyl, or heteroaryl;
C is aryl, heterocycle, cycloalkyl, bicycle, or heteroaryl;
D is aryl or heteroaryl;
X is CH, CR⁷⁵, or N;
Y is CH, CR⁷⁵, or N;
Z is selected from —O—, —NR⁶—, —S—, —S(O)—, —S(O)₂—, and —CR^{3a}R^{3b}—; and
R⁹⁹, R¹⁰⁰, R¹⁰¹, and R¹⁰² are independently selected at each instance from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅ wherein the optional substituent is selected such that a stable compound results.
14. The compound of embodiment 13, wherein the compound is of formula:

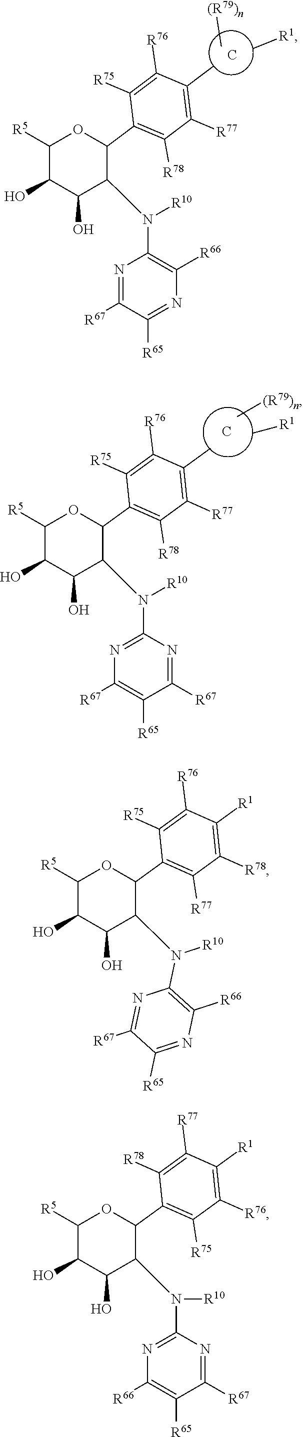

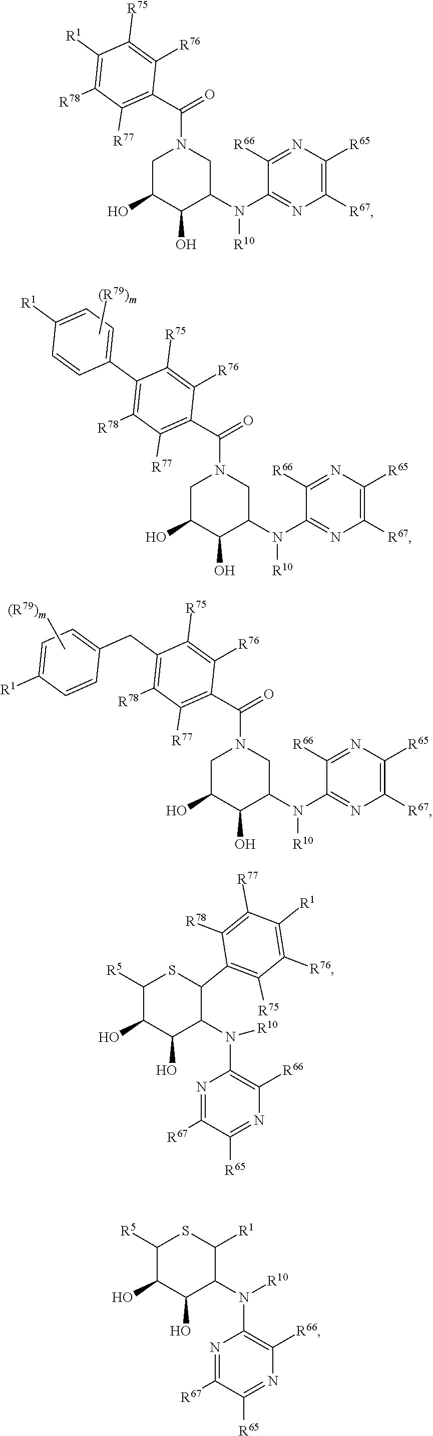

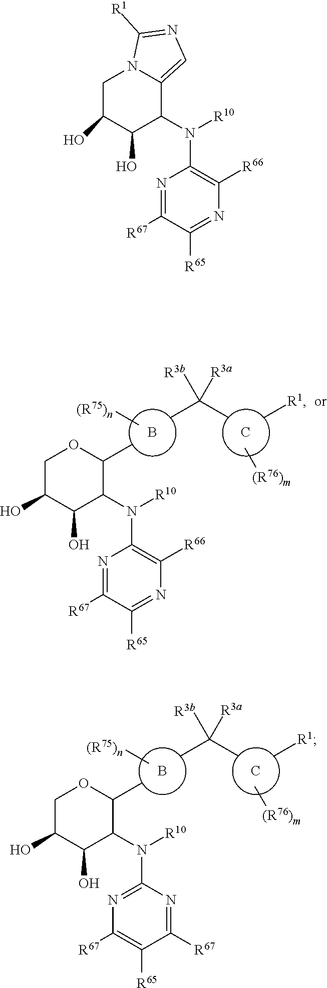

or a pharmaceutically acceptable salt thereof.
15. The compound of embodiment 13, wherein the compound is of formula:

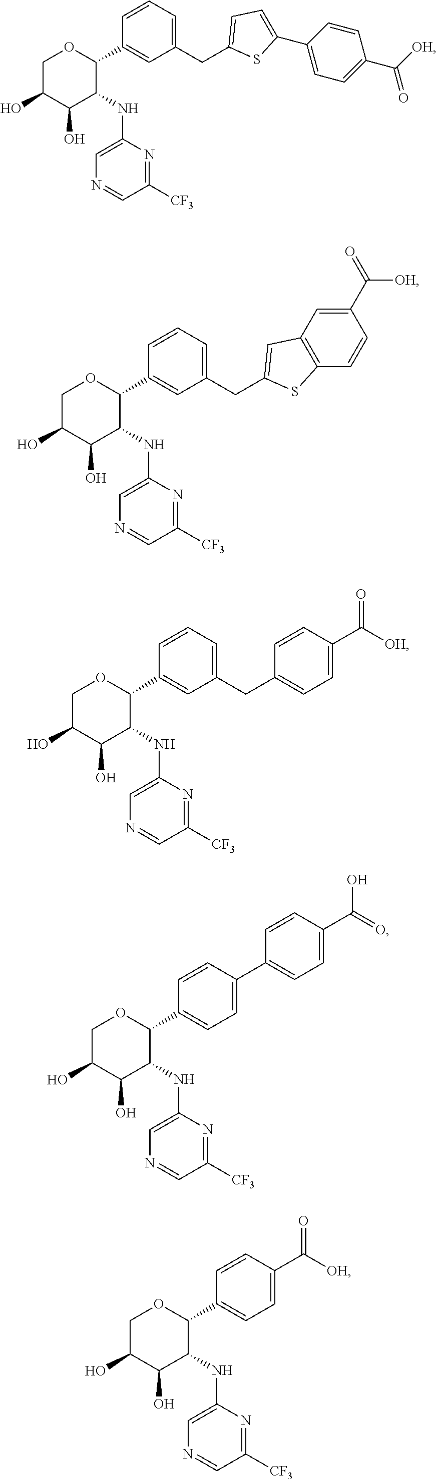 [SMILES: O=C(O)c1ccc(-c2ccc(Cc3cccc([C@H]4OC[C@H](O)[C@H](O)[C@H]4Nc4cncc(C(F)(F)F)n4)c3)s2)cc1 ; O=C(O)c1ccc2sc(Cc3cccc([C@H]4OC[C@H](O)[C@H](O)[C@H]4Nc4cncc(C(F)(F)F)n4)c3)cc2c1 ; O=C(O)c1ccc(Cc2cccc([C@H]3OC[C@H](O)[C@H](O)[C@H]3Nc3cncc(C(F)(F)F)n3)c2)cc1 ; O=C(O)c1ccc(-c2ccc([C@H]3OC[C@H](O)[C@H](O)[C@H]3Nc3cncc(C(F)(F)F)n3)cc2)cc1 ; O=C(O)c1ccc([C@H]2OC[C@H](O)[C@H](O)[C@H]2Nc2cncc(C(F)(F)F)n2)cc1]

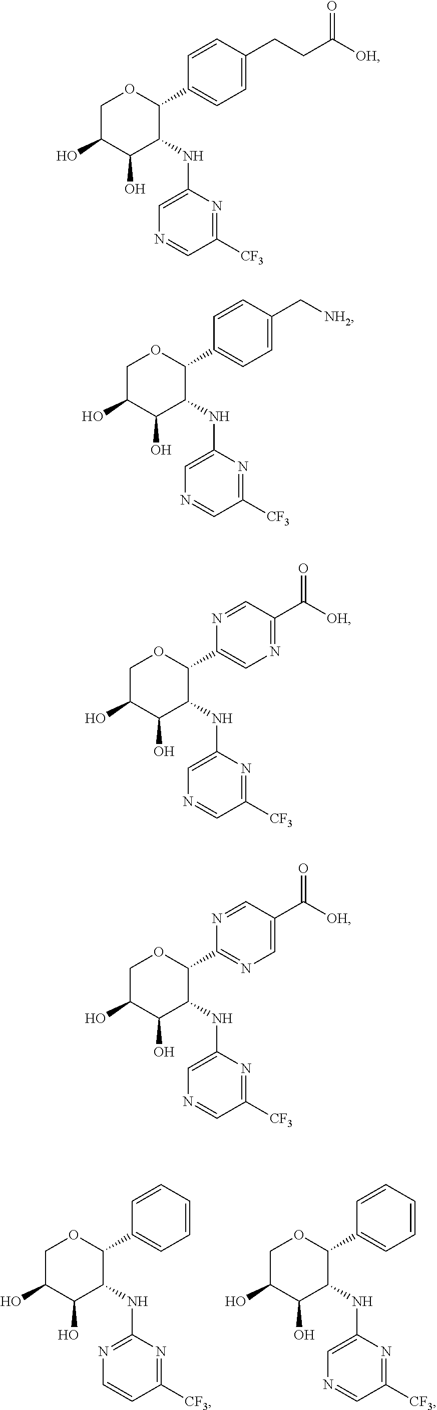

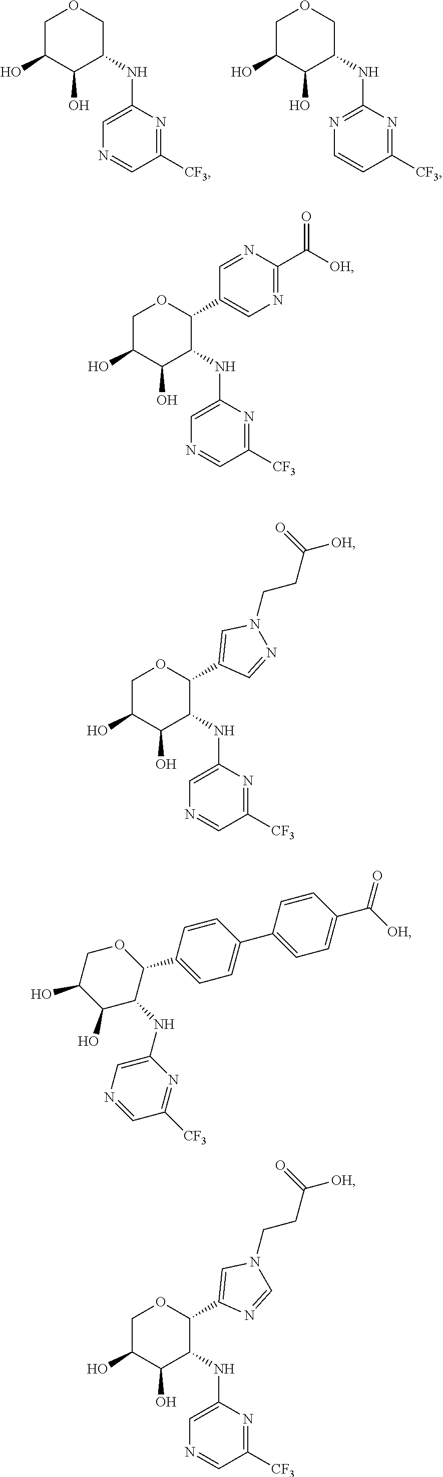 [SMILES: O[C@@H]1[C@@H](Nc2cncc(C(F)(F)F)n2)COC[C@@H]1O ; O[C@@H]1[C@@H](Nc2nccc(C(F)(F)F)n2)COC[C@@H]1O ; O=C(O)c1ncc([C@H]2OC[C@H](O)[C@H](O)[C@H]2Nc2cncc(C(F)(F)F)n2)cn1 ; O=C(O)CCn1cc([C@H]2OC[C@H](O)[C@H](O)[C@H]2Nc2cncc(C(F)(F)F)n2)cn1 ; O=C(O)c1ccc(-c2ccc([C@H]3OC[C@H](O)[C@H](O)[C@H]3Nc3cncc(C(F)(F)F)n3)cc2)cc1 ; O=C(O)CCn1cnc([C@H]2OC[C@H](O)[C@H](O)[C@H]2Nc2cncc(C(F)(F)F)n2)c1]

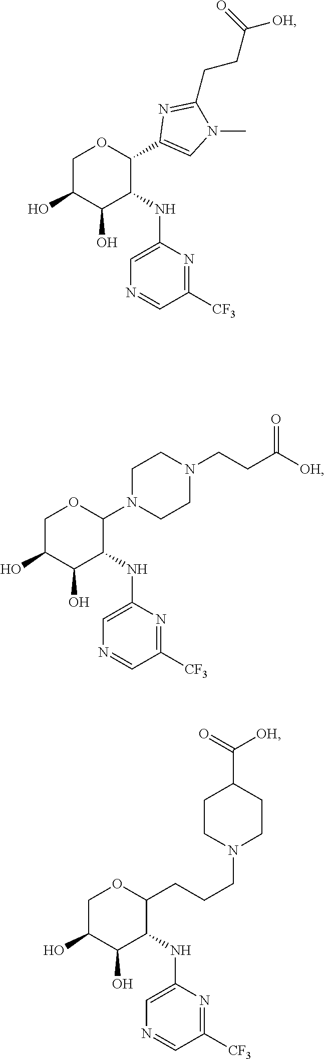 [SMILES: Cn1cc([C@H]2OC[C@H](O)[C@H](O)[C@H]2Nc2cncc(C(F)(F)F)n2)nc1CCC(=O)O ; O=C(O)CCN1CCN(C2OC[C@H](O)[C@H](O)[C@H]2Nc2cncc(C(F)(F)F)n2)CC1 ; O=C(O)C1CCN(CCCC2OC[C@H](O)[C@H](O)[C@H]2Nc2cncc(C(F)(F)F)n2)CC1]

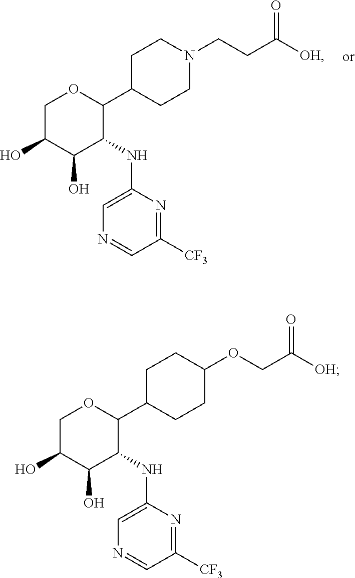 [SMILES: O=C(O)CCN1CCC(C2OC[C@H](O)[C@H](O)[C@H]2Nc2cncc(C(F)(F)F)n2)CC1 ; O=C(O)COC1CCC(C2OC[C@H](O)[C@H](O)[C@H]2Nc2cncc(C(F)(F)F)n2)CC1]

or a pharmaceutically acceptable salt thereof.
16. The compound of embodiment 13, wherein the compound is of formula:

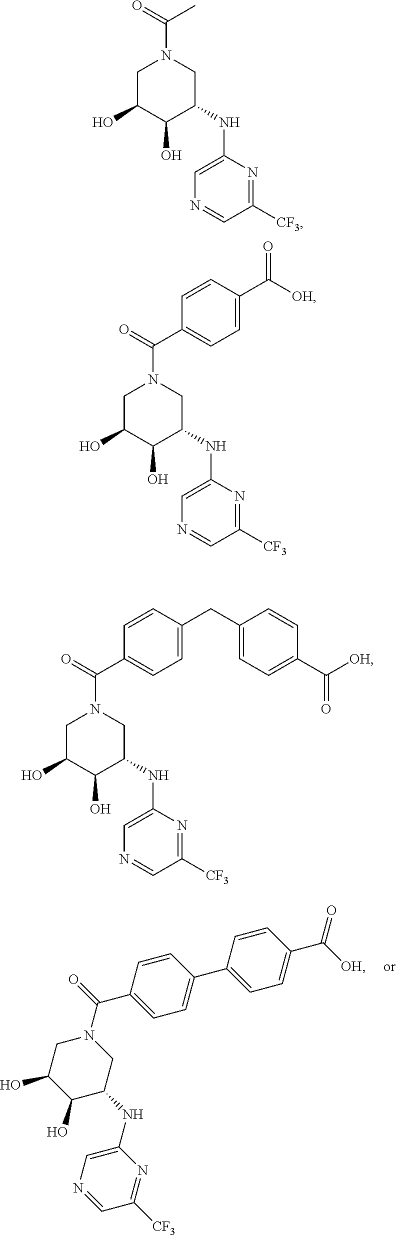

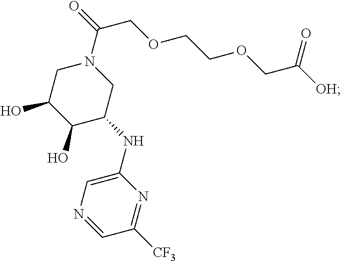 [SMILES: O=C(O)COCCOCC(=O)N1C[C@H](Nc2cncc(C(F)(F)F)n2)[C@@H](O)[C@@H](O)C1]

or a pharmaceutically acceptable salt thereof.
17. The compound of embodiment 13, wherein the compound is of formula:

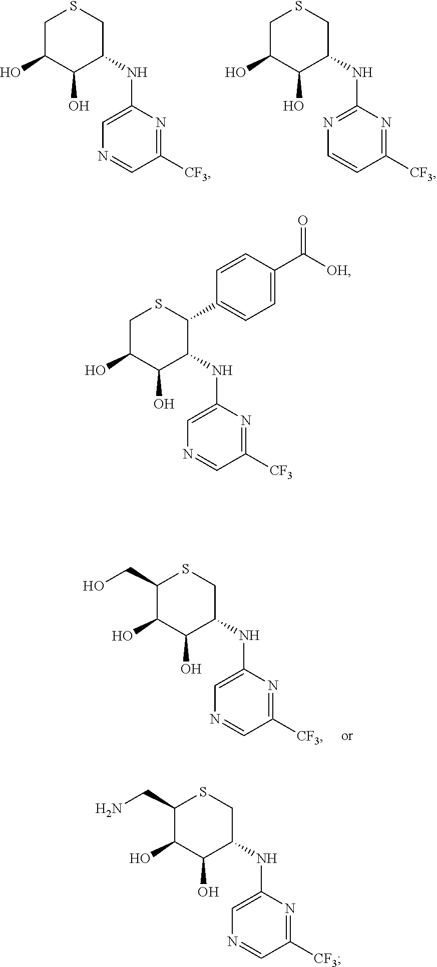 [SMILES: O[C@@H]1[C@@H](Nc2cncc(C(F)(F)F)n2)CSC[C@@H]1O ; O[C@@H]1[C@@H](Nc2nccc(C(F)(F)F)n2)CSC[C@@H]1O ; O=C(O)c1ccc([C@H]2SC[C@H](O)[C@H](O)[C@H]2Nc2cncc(C(F)(F)F)n2)cc1 ; OC[C@H]1SC[C@H](Nc2cncc(C(F)(F)F)n2)[C@@H](O)[C@H]1O ; NC[C@H]1SC[C@H](Nc2cncc(C(F)(F)F)n2)[C@@H](O)[C@H]1O]

or a pharmaceutically acceptable salt thereof.
18. The compound of embodiment 13 of the formula:

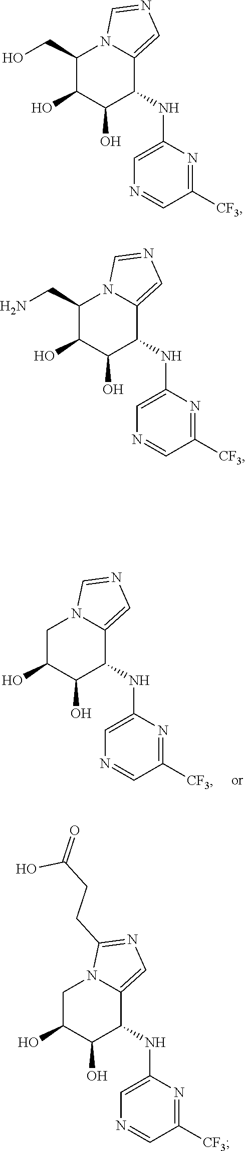 [SMILES: OC[C@@H]1[C@H](O)[C@H](O)[C@@H](Nc2cncc(C(F)(F)F)n2)c2cncn21 ; NC[C@@H]1[C@H](O)[C@H](O)[C@@H](Nc2cncc(C(F)(F)F)n2)c2cncn21 ; O[C@H]1[C@@H](O)Cn2cncc2[C@@H]1Nc1cncc(C(F)(F)F)n1 ; O=C(O)CCc1ncc2n1C[C@H](O)[C@H](O)[C@H]2Nc1cncc(C(F)(F)F)n1]

or a pharmaceutically acceptable salt thereof
19. A compound of the formula:

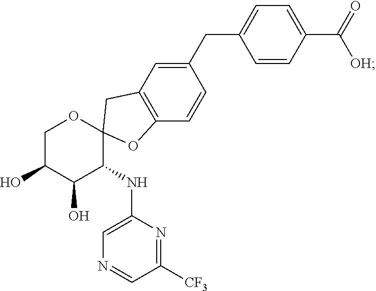 [SMILES: O=C(O)c1ccc(Cc2ccc3c(c2)CC2(OC[C@H](O)[C@H](O)[C@H]2Nc2cncc(C(F)(F)F)n2)O3)cc1]

or a pharmaceutically acceptable salt thereof.
1. An ASGPR-binding extracellular protein degrader compound of the formula.

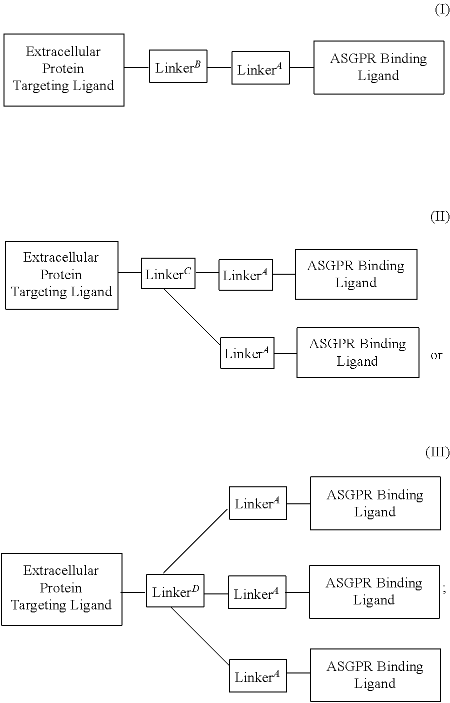

or a pharmaceutically acceptable salt thereof;
wherein the ASGPR Binding Ligand is selected from:

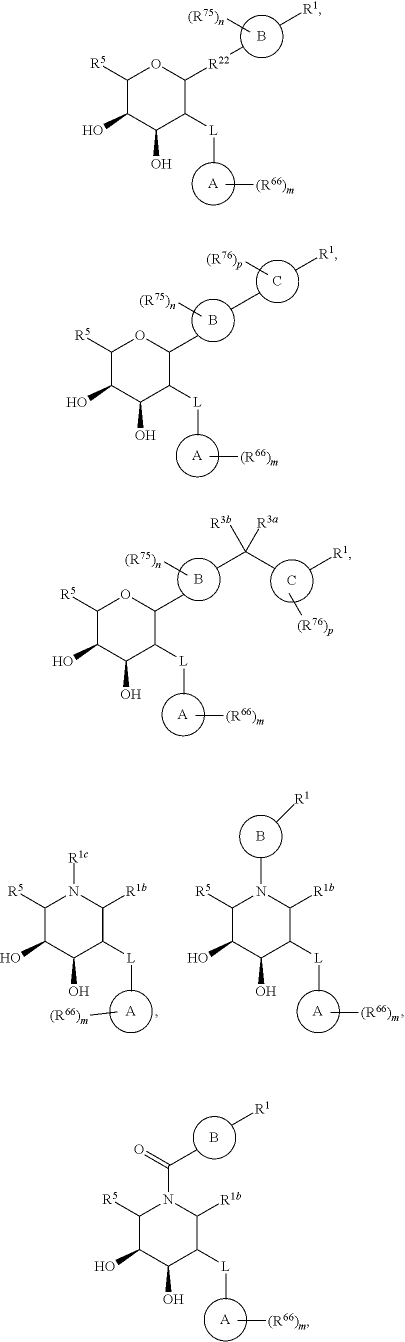

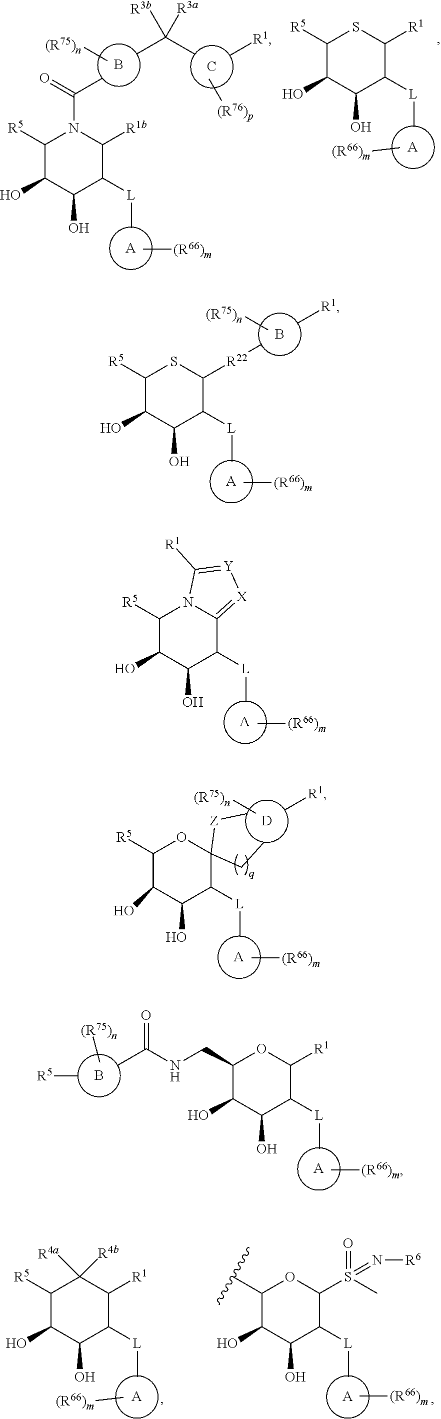

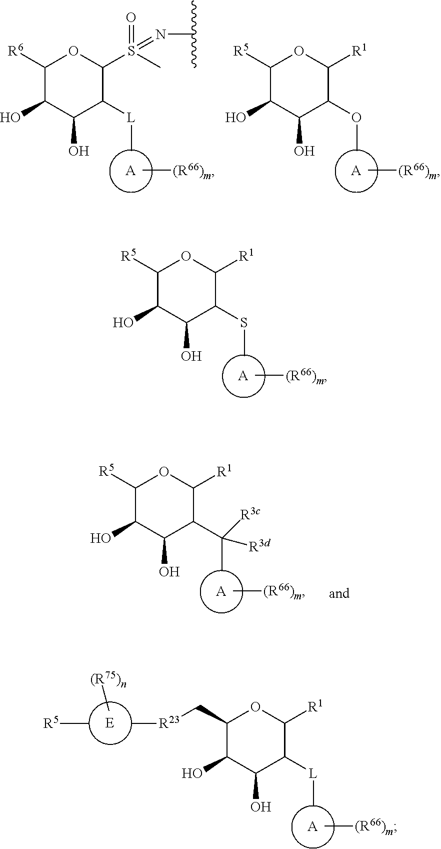

R¹, R^{1b}, and R⁵ are independently selected from hydrogen, C₀-C₆alkyl-cyano, alkyl, alkenyl, alkynyl, haloalkyl, F, Cl, Br, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, and C₀-C₆alkylN₃, each of which except hydrogen, F, Cl, and Br is optionally substituted with 1, 2, 3, or 4 substituents independently selected at each occurrence from R⁹⁹;
R^{1c} is selected from hydrogen, alkyl, alkenyl, alkynyl, haloalkyl, —C(O)R³, —S(O)R³, —C(S)R³, and —S(O)₂R³;
wherein one of Ri R^{b}, R¹⁰, and R⁵ is replaced with a bond to Linker^{A};
L is selected from,

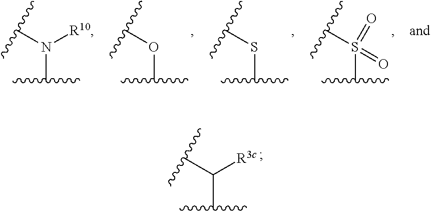

R³, R^{3a}, R^{3b}, R^{3c}, and R^{3d} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R^{4a} is selected from hydrogen, alkyl, haloalkyl, and halogen;
R^{4b} is selected from hydrogen, alkyl, haloalkyl, halogen, C₀-C₆alkyl-OR⁶, C₀-C₆alkyl-SR⁶, and C₀-C₆alkyl-NR⁶R⁷;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
R²² is selected from bond, C₁-C₄ alkyl, C₁-C₄ haloalkyl, C₂-C₄ alkenyl, and C₂-C₄ alkynyl;
R²³ is selected from bond,

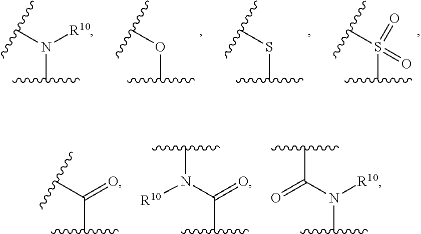

C₁-C₄ alkyl, C₁-C₄ haloalkyl, C₂-C₄ alkenyl, and C₂-C₄ alkynyl;
R⁶⁶ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, 3, or 4 substituents independently selected at each occurrence from R¹⁰⁰;
R⁷⁵ and R⁷⁶ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R¹⁰²;
n, m, and p are independently 0, 1, 2, 3, or 4, as allowed by valence;
q is 1, 2, or 3;
\textcircled{A} O is heteroaryl;
\textcircled{B} is aryl, heterocycle, cycloalkyl, or heteroaryl;
\textcircled{C} is aryl, heterocycle, cycloalkyl, bicycle, or heteroaryl;
\textcircled{D} is aryl or heteroaryl;
\textcircled{E} is a bicycle or spirocycle; non-limiting examples of \textcircled{E} include

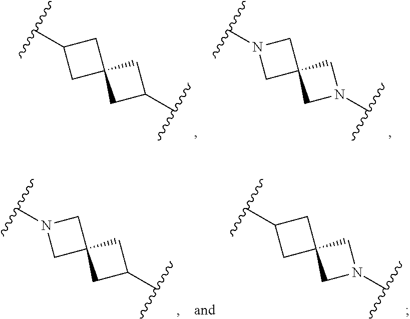

X is CH, CR⁷⁵, or N;
Y is CH, CR⁷⁵, or N;
Z is selected from —O—, —NR⁶—, —S—, —S(O)—, —S(O)₂—, and —CR^{3a}R^{3b}—;
R⁹⁹, R¹⁰⁰, and R¹⁰² are independently selected at each instance from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅ wherein the optional substituent is selected such that a stable compound results;
Linker^{A} is a bond or a moiety that covalently links Linker^{B}, Linker^{C}, or Linker^{D} to the ASGPR Binding Ligand;
Linker^{B} is a bond or a moiety that covalently links Linker^{A} to an Extracellular Protein Targeting Ligand;
Linker^{C} is a chemical group that links each Linker^{A} to the Extracellular Protein Targeting Ligand;
Linker^{D} is a chemical group that links each Linker^{A} to the Extracellular Protein Targeting Ligand; and
Extracellular Protein Targeting Ligand is a Ligand that binds to an extracellular protein.
2. The compound of embodiment 1, wherein the ASPGR Binding Ligand is selected from:

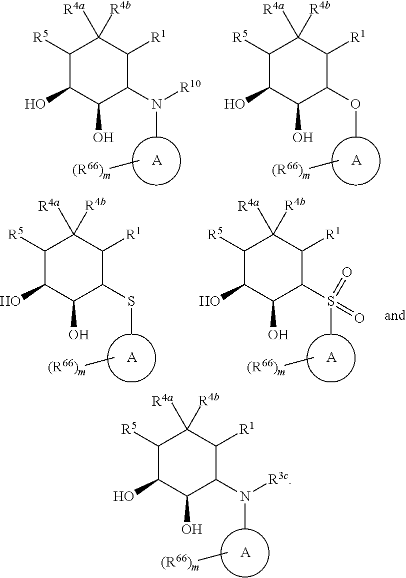

3. The compound of embodiment 1 or embodiment 2, wherein R^{4a} is hydrogen.
4. The compound of embodiment 1 or embodiment 2, wherein R^{4a} is F.
5. The compound of any one of embodiments 1-4, wherein R^{4b} is hydrogen.
6. The compound of any one of embodiments 1-4, wherein R^{4b} is F.
7. The compound of embodiment 1, wherein the ASPGR Binding Ligand is selected from:

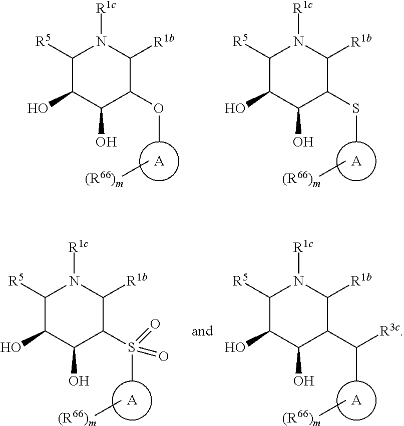

8. The compound of embodiment 1 or embodiment 7, wherein R^{1c} is a bond to Linker^{A}.
9. The compound of embodiment 1 or embodiment 7, wherein R^{1c} is hydrogen.
10. The compound of embodiment 1, wherein the ASPGR Binding Ligand is selected from:

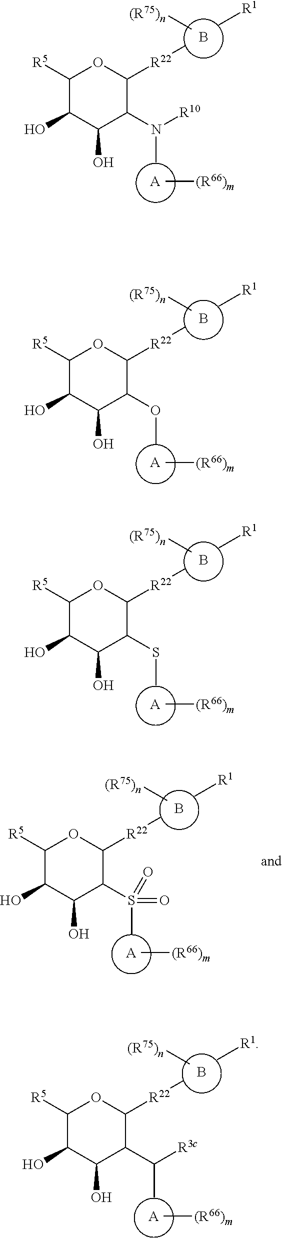

11. The compound of embodiment 1 or embodiment 10, wherein R²² is bond.
12. The compound of embodiment 1 or embodiment 10, wherein R²² is C₁-C₄ alkyl.
13. The compound of embodiment 1 or embodiment 10, wherein R²² is C₁-C₄ haloalkyl.
14. The compound of any one of embodiments 10-13, wherein \textcircled{B} is phenyl.
15. The compound of embodiment 1, wherein the ASPGR Binding Ligand is selected from:

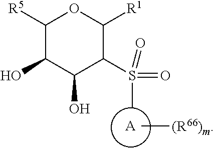

16. The compound of any one of embodiments 1-15, wherein

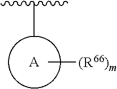

is selected from

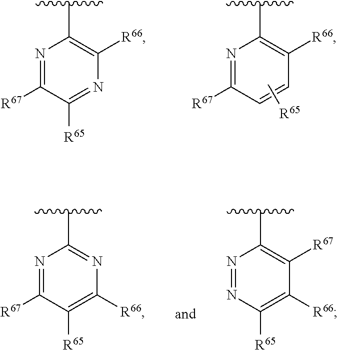

wherein R⁶⁵ and R⁶⁷ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, 3, or 4 substituents independently selected at each occurrence from R¹⁰⁰.
17. The compound of embodiment 16, wherein R⁶⁵ is hydrogen.
18. The compound of embodiment 16, wherein R⁶⁵ is halogen.
19. The compound of embodiment 16, wherein R⁶⁵ is F or Cl.
20. The compound of embodiment 16, wherein R⁶⁵ is C₁-C₄alkyl.
21. The compound of embodiment 16, wherein R⁶⁵ is C₁-C₄haloalkyl.
22. The compound of embodiment 16, wherein R⁶⁵ is CF₃.
23. The compound of any one of embodiments 16-22, wherein R⁶⁶ is hydrogen.
24. The compound of any one of embodiments 16-22, wherein R⁶⁶ is halogen.
25. The compound of any one of embodiments 16-22, wherein R⁶⁶ is F or Cl.
26. The compound of any one of embodiments 16-22, wherein R⁶⁶ is C₁-C₄alkyl.
27. The compound of any one of embodiments 16-22, wherein R⁶⁶ is C₁-C₄haloalkyl.
28. The compound of any one of embodiments 16-22, wherein R⁶⁶ is CF₃.
29. The compound of any one of embodiments 16-28, wherein R⁶⁷ is hydrogen.
30. The compound of any one of embodiments 16-28, wherein R⁶⁷ is halogen.
31. The compound of any one of embodiments 16-28, wherein R⁶⁷ is F or Cl.
32. The compound of any one of embodiments 16-28, wherein R⁶⁷ is C₁-C₄alkyl.
33. The compound of any one of embodiments 16-28, wherein R⁶⁷ is C₁-C₄haloalkyl.
34. The compound of any one of embodiments 16-28, wherein R⁶⁷ is CF₃.
35. The compound of any one of embodiments 1-15, wherein

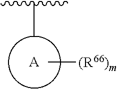

is

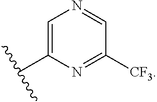 [SMILES: *c1cncc(C(F)(F)F)n1]

36. The compound of any one of embodiments 1-15, wherein

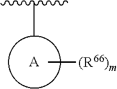

is

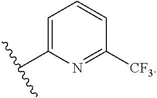 [SMILES: *c1cccc(C(F)(F)F)n1]

37. The compound of embodiment 1, wherein the ASPGR Binding Ligand is selected from:

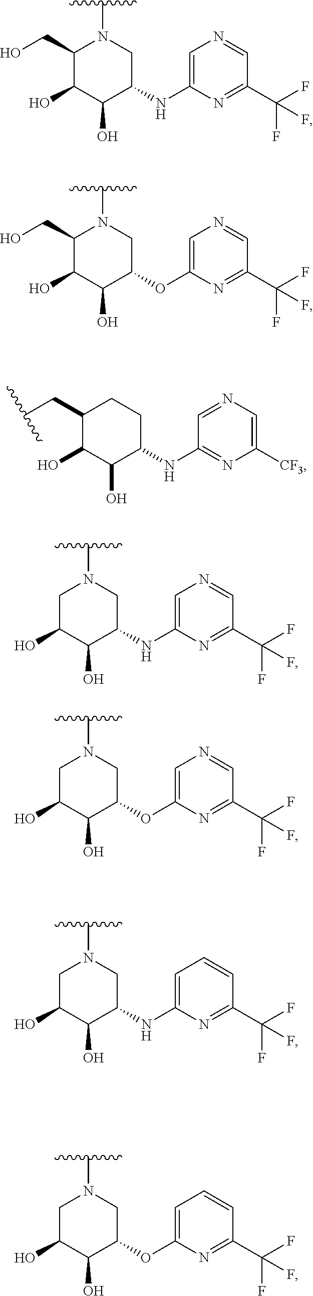

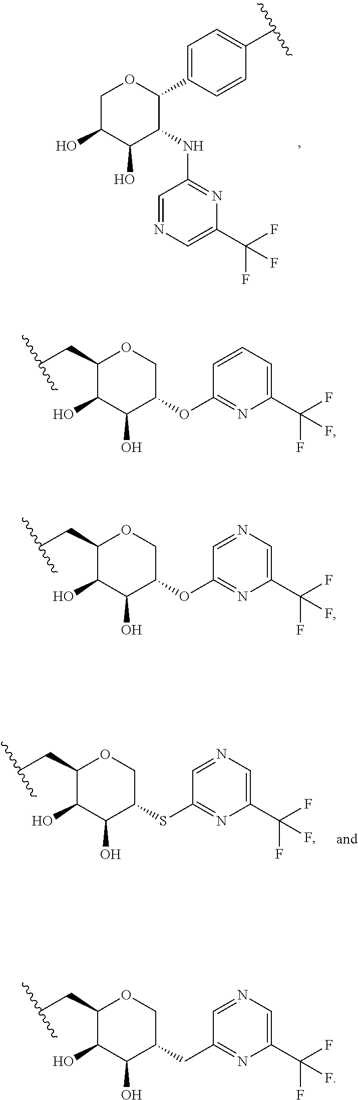 [SMILES: *c1ccc([C@H]2OC[C@H](O)[C@H](O)[C@H]2Nc2cncc(C(F)(F)F)n2)cc1 ; *C[C@H]1OC[C@H](Oc2cccc(C(F)(F)F)n2)[C@@H](O)[C@H]1O ; *C[C@H]1OC[C@H](Oc2cncc(C(F)(F)F)n2)[C@@H](O)[C@H]1O ; *C[C@H]1OC[C@H](Sc2cncc(C(F)(F)F)n2)[C@@H](O)[C@H]1O ; *C[C@H]1OC[C@H](Cc2cncc(C(F)(F)F)n2)[C@@H](O)[C@H]1O]

38. The compound of embodiment 1, wherein the ASPGR Binding Ligand is a compound selected from:

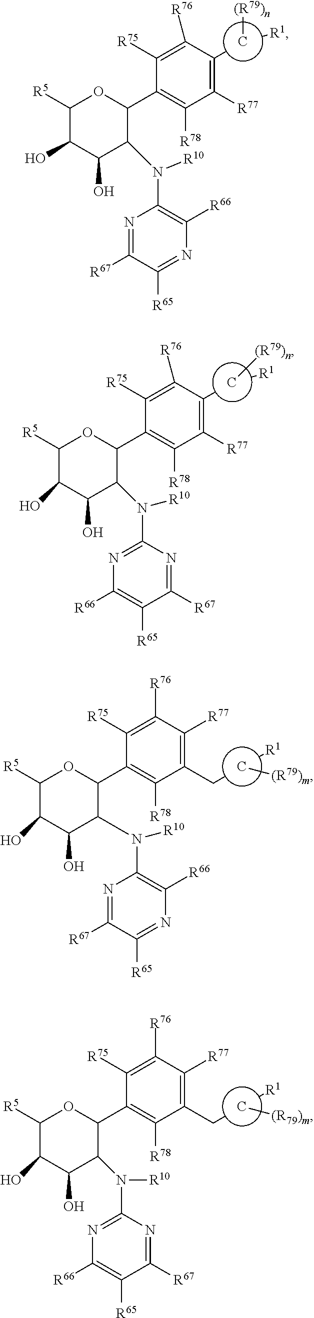

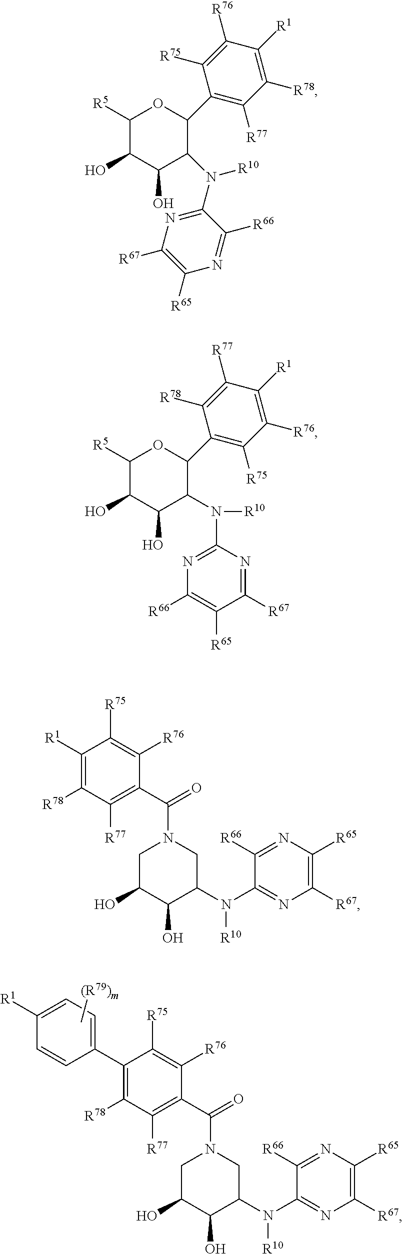

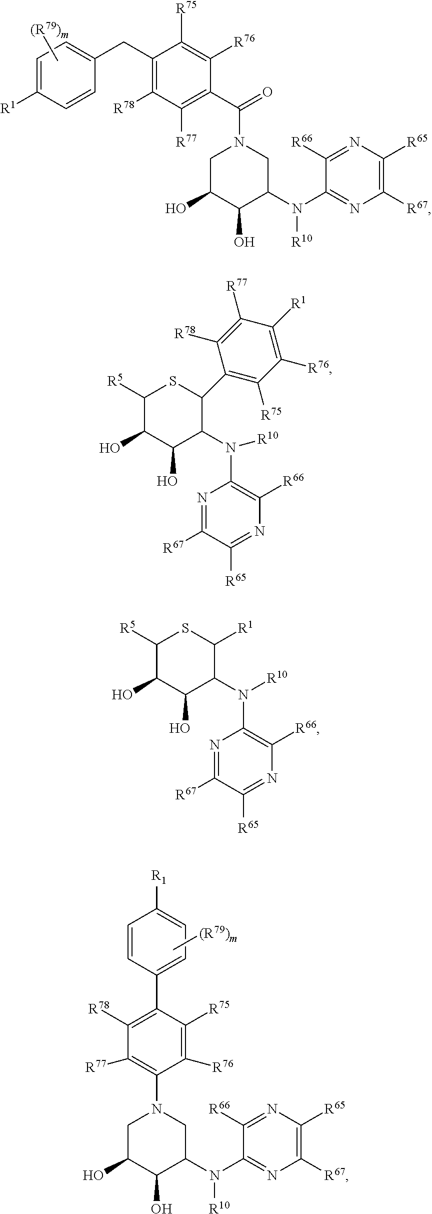

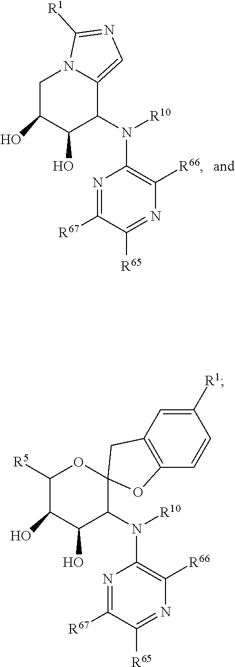

wherein R^{6′} and R⁶⁷ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, 3, or 4 substituents independently selected at each occurrence from R¹⁰⁰; and
R⁷⁷, R⁷⁸, and R⁷⁹ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, 3, or 4 substituents independently selected at each occurrence from R¹⁰².
39. The compound of embodiment 1, wherein the compound is of Formula

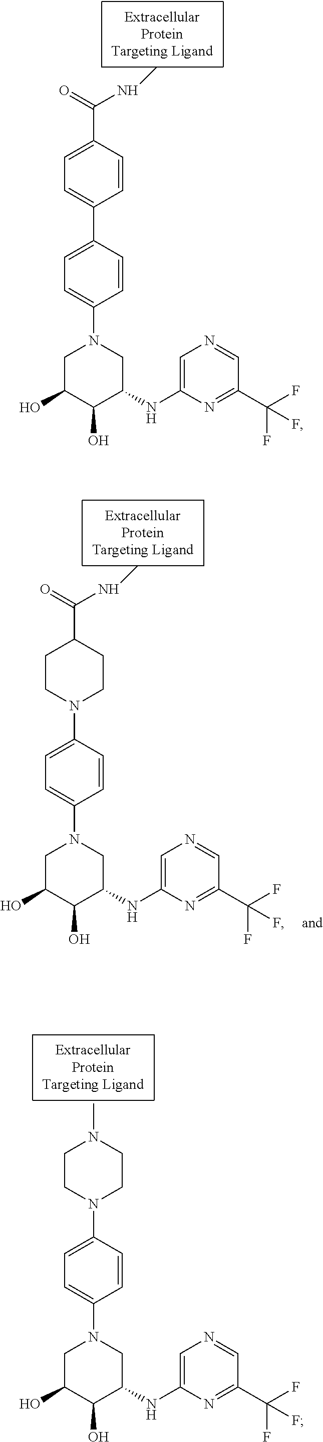 [SMILES: CNC(=O)c1ccc(-c2ccc(N3C[C@H](Nc4cncc(C(F)(F)F)n4)[C@@H](O)[C@@H](O)C3)cc2)cc1 ; CNC(=O)C1CCN(c2ccc(N3C[C@H](Nc4cncc(C(F)(F)F)n4)[C@@H](O)[C@@H](O)C3)cc2)CC1 ; CN1CCN(c2ccc(N3C[C@H](Nc4cncc(C(F)(F)F)n4)[C@@H](O)[C@@H](O)C3)cc2)CC1]

or a pharmaceutically acceptable salt thereof.
40. The compound of embodiment 1, wherein the compound is of Formula:

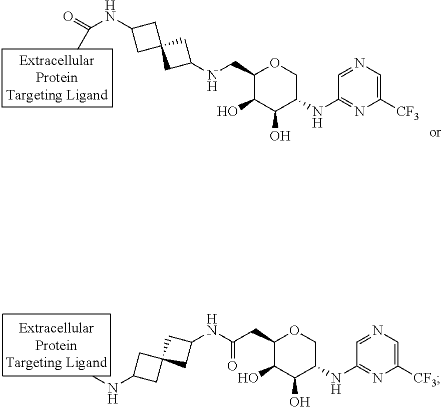

or a pharmaceutically acceptable salt thereof.
41. The compound of any one of embodiments 1-40, wherein the Extracellular Protein Targeting Ligand binds IgG.
42. The compound of embodiment 41, wherein the IgG targeting Extracellular Protein Targeting Ligand is Fc-BP-2.
43. The compound of embodiment 41, wherein the IgG targeting Extracellular Protein Targeting Ligand is Fc-III.
44. The compound of any one of embodiments 1-40, wherein the Extracellular Protein Targeting Ligand binds IgM.
45. The compound of any one of embodiments 1-40, wherein the Extracellular Protein Targeting Ligand targets IgE.
46. The compound of any one of embodiments 1-40, wherein the Extracellular Protein Targeting Ligand targets TNF-α.
47. The compound of any one of embodiments 1-40, wherein the Extracellular Protein Targeting Ligand targets IL-1b.
48. The compound of any one of embodiments 1-40, wherein the Extracellular Protein Targeting Ligand targets IL-2.
49. The compound of any one of embodiments 1-40, wherein the Extracellular Protein Targeting Ligand targets IL-6.
50. The compound of any one of embodiments 1-40, wherein the Extracellular Protein Targeting Ligand targets IFN-7.
51. The compound of any one of embodiments 1-40, wherein the Extracellular Protein Targeting Ligand targets VEGF.
52. The compound of any one of embodiments 1-40, wherein the Extracellular Protein Targeting Ligand targets TGF-b1.
53. The compound of any one of embodiments 1-40, wherein the Extracellular Protein Targeting Ligand targets PCSK-9.
54. The compound of any one of embodiments 1-53 wherein Linker^{B} is:

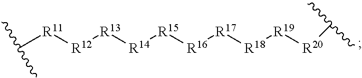

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, 3, or 4 substituents independently selected from R²¹;
n is independently selected at each instance from 0, 1, 2, 3, 4, 5, 6, 7, 8, 9, or 10; and
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR'S(O)R³, haloalkyl, aryl, heteroaryl, heterocyclyl, —SR³, —C(O)OR³, —C(O)NR⁶NR⁷, —OR³,

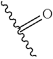 [SMILES: *=O]

and heterocycle.
55. The compound of embodiment 54, wherein R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.
56. The compound of embodiment 54 or 55, wherein 1, 2, 3, or 4 of R¹¹, R¹², R¹³, R¹⁴, R¹, R¹⁶, R¹⁷, R¹⁸, and R¹⁹ is bond.
57. The compound of embodiment 54 or 55, wherein 1, 2, 3, or 4 of R¹¹, R¹², R¹³, R¹⁴, R¹, R¹⁶, R¹⁷, R¹⁸, and R¹⁹ is an amino acid.
58. The compound of embodiment 54, wherein Linker^{B} is selected from:

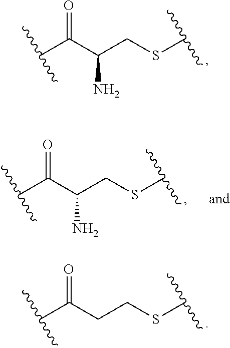

59. The compound of embodiment 54, wherein Linker^{B} is selected from:

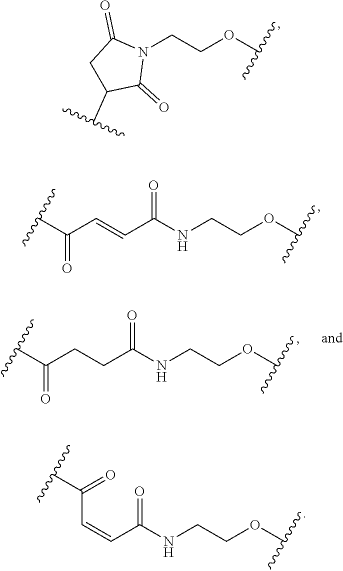 [SMILES: *OCCN1C(=O)CC(*)C1=O ; *OCCNC(=O)/C=C/C(*)=O ; *OCCNC(=O)CCC(*)=O ; *OCCNC(=O)/C=C\C(*)=O]

60. The compound of any one of embodiments 1-59, wherein the compound is of Formula:

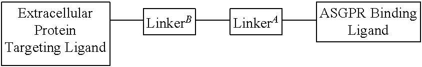

or a pharmaceutically acceptable salt thereof.
61. The compound of any one of embodiments 1-53, wherein Linker^{C} is selected from:

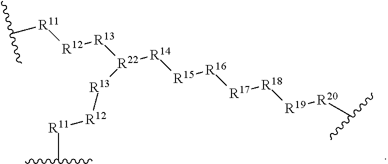

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR'S(O)R³, haloalkyl, aryl, heteroaryl, heterocyclyl, —SR³, —C(O)OR³, —C(O)NR⁶NR⁷, —OR³,

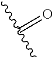 [SMILES: *=O]

and heterocycle; and
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, 3, or 4 substituents independently selected from R²¹.
62. The compound of embodiment 61, wherein R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.
63. The compound of embodiment 61 or 62, wherein Linker^{C} is selected from:

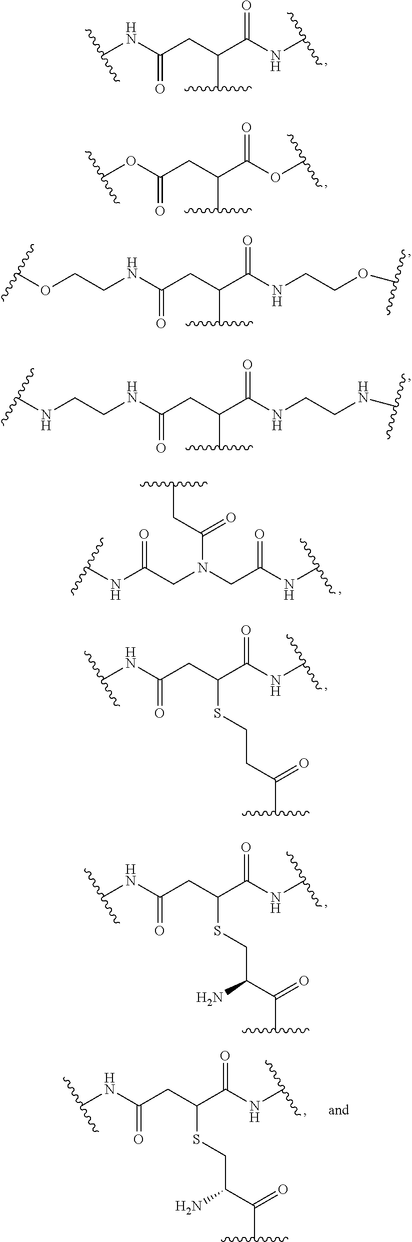

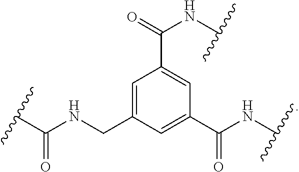 [SMILES: *NC(=O)c1cc(CNC(*)=O)cc(C(=O)N*)c1]

64. The compound of any one of embodiments 61-63, wherein the compound is of Formula:

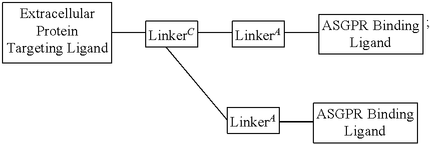

or a pharmaceutically acceptable salt thereof.
65. The compound of any one of embodiments 1-53, wherein Linker^{D} is selected from:

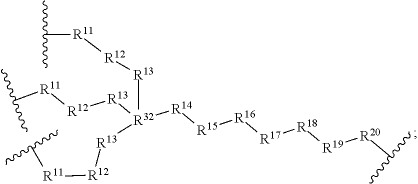

wherein:
R³² is independently at each occurrence selected from the group consisting of alkyl, N⁺X⁻, —C—, alkenyl, haloalkyl, aryl, heterocycle, and heteroaryl, each of which is optionally substituted with 1, 2, 3, or 4 substituents independently selected from R²¹; and X⁻ is an anionic group.
66. A pharmaceutical composition comprising a compound of any one of embodiments 1 to 65 and a pharmaceutically acceptable carrier.
67. A method of treating a disorder mediated by an Extracellular Protein comprising administering an effective amount of a compound of any one of embodiments 1 to 65 that includes an Extracellular Protein Targeting Ligand that binds to the Extracellular Protein, or a pharmaceutically acceptable salt thereof, to a patient in need thereof.
68. The method of embodiment 67, wherein the extracellular protein is IgA and the disorder is selected from IgA nephropathy (Berger's disease), celiac disease, Crohn's disease, Henoch-Sconiein purpura (HSP), liner IgA bullous dermatosis, IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, α-chain disease, IgA monoclonal gammopathy, monoclonal gammopathy of undetermined significance (MGUS), and linear IgA bullous dermatosis.
69. The method of embodiment 67, wherein the extracellular protein is IgG and the disorder is selected from type 1 autoimmune pancreatitis, interstitial nephritis, Riedel's thyroiditis, storiform fibrosis, Mikulicz's disease, Küttner's tumor, inflammatory pseudotumors, mediastinal fibrosis, retroperitoneal fibrosis (Ormond's disease), aortitis, periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, thyroid eye disease, chronic inflammatory demyelinating polyneuropathy, warm autoimmune hemolytic anemia, ankylosing spondylitis, primary Sjögren's syndrome, psoriatic arthritis, and systemic lupus erythematosus (SLE), sclerosing cholangitis, and IgG monoclonal gammopathy, monoclonal gammopathy of undetermined significance (MGUS).
70. The method of embodiment 67, wherein the extracellular protein is IgE and the disorder is selected from atopic asthma, allergic rhinitis, atopic dermatitis, IgE-mediated food allergy, IgE-mediated animal allergies, allergic conjunctivitis, allergic urticaria, anaphylactic shock, nasal polyposis, keratoconjunctivitis, mastocytosis, and eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, and monoclonal gammopathy of undetermined significance (MGUS).
71. The method of embodiment 67, wherein the disorder is dementia or Alzheimer's disease.
72. The method of embodiment 67, wherein the extracellular protein is TNF-α and the disorder is selected from rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.
73. The method of embodiment 67, wherein the extracellular protein is IL-2 and the disorder is selected from host versus graft rejection in transplants and autoimmune disorders.
74. The method of embodiment 67, wherein the extracellular protein is IL-6 and the disorder is selected from Castleman's disease, metastatic castration-associated prostate cancer, renal cell carcinoma, large-cell lung carcinoma, ovarian cancer, rheumatoid arthritis, and asthma.
75. The method of embodiment 67, wherein the extracellular protein is IFN-γ and the disorder is selected from rheumatoid arthritis, multiple sclerosis (MS), corneal transplant rejection, and various autoimmune skin diseases such as psoriasis, alopecia areata, vitiligo, and acne vulgaris.
76. The method of embodiment 67, wherein the disorder is a cancer.
77. A compound of formula:

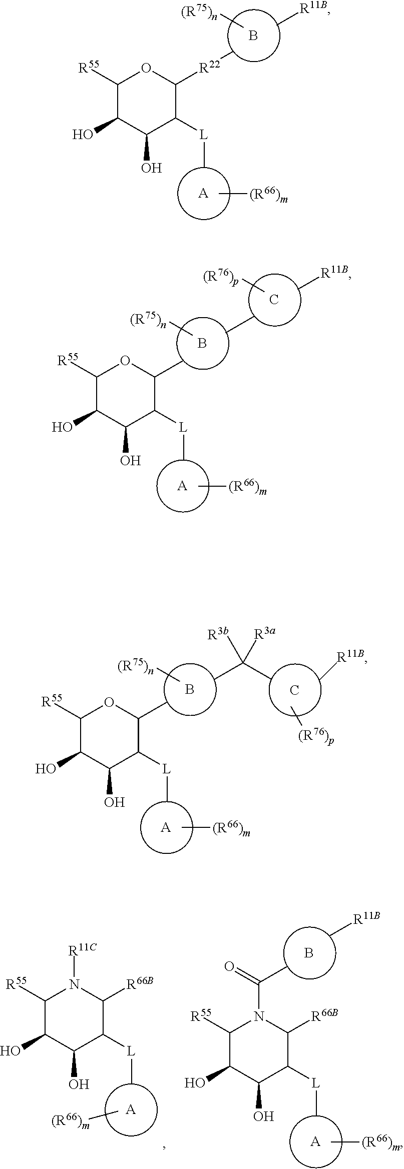

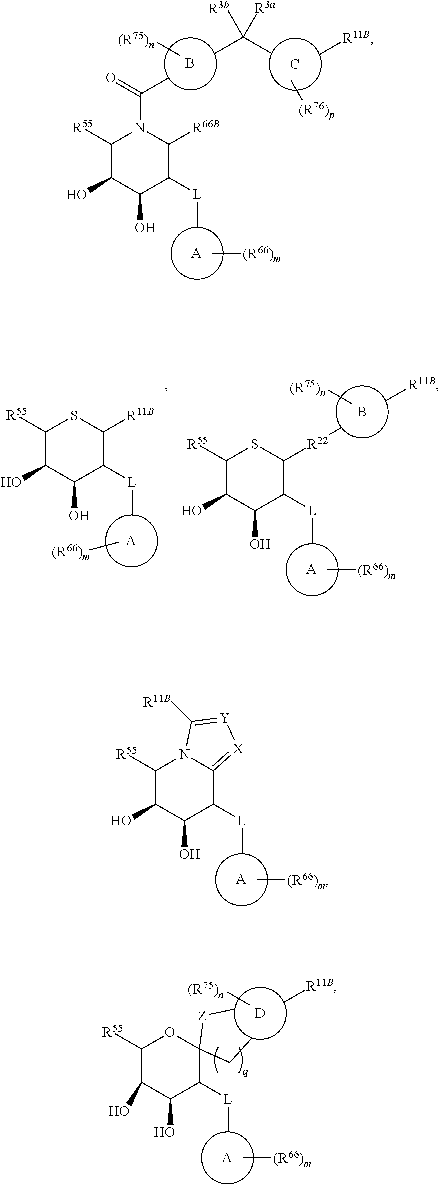

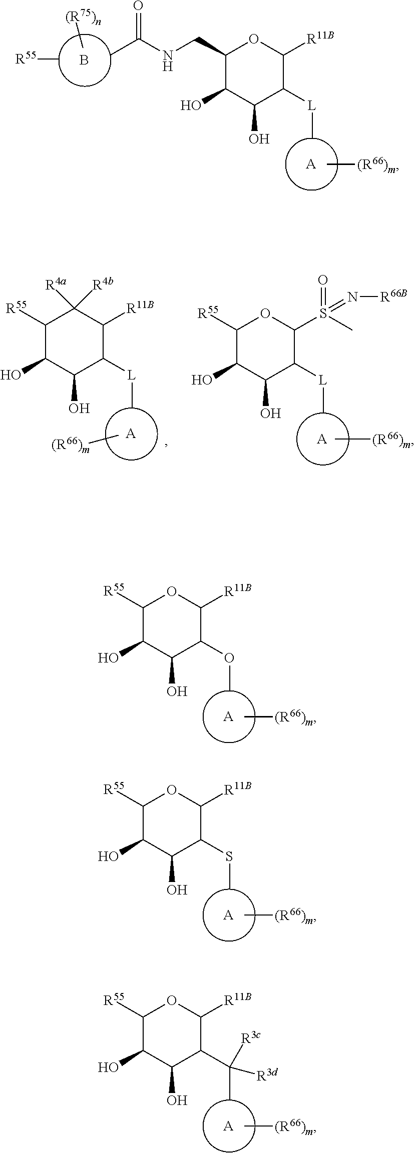

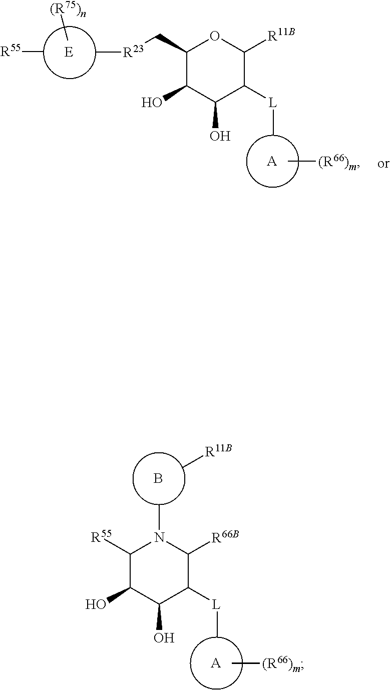

or a salt thereof;
wherein:
R^{11B}, R⁵⁵, and R^{66B} are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkylN₃, and R¹¹⁰ each of which is optionally substituted with 1, 2, 3, or 4 substituents independently selected at each occurrence from R⁹⁹;
R^{11C} is selected from hydrogen, alkyl, alkenyl, alkynyl, haloalkyl, —C(O)R³, —S(O)R³, —C(S)R³, —S(O)₂R³, and R¹¹⁰;
and wherein in typical embodiments where R^{11B} is attached to a nitrogen then R^{11B} is selected from hydrogen, alkyl, alkenyl, alkynyl, haloalkyl, aryl, arylalkyl, heteroaryl, heteroarylalkyl, heterocycle, heterocycloalkyl, C₀-C₆alkyl-C(O)R³, C₀-C₆alkyl-S(O)R³, C₀-C₆alkyl-C(S)R³, and C₀-C₆alkyl-S(O)₂R³;
R¹¹⁰ is:

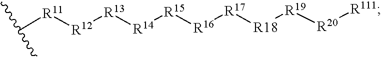

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, 3, or 4 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R¹¹¹ is independently selected from the group consisting of hydrogen, alkyl, cycloakyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkyl-O—P(O)₂R³, C₀-C₆alkyl-O—P(O)₃—R³, C₀-C₆alkyl-O—P(O)(OR³)₂, and C₀-C₆alkylN₃, each of which is optionally substituted with 1, 2, 3, or 4 substituents independently selected at each occurrence from R¹⁰³;
R¹⁰³ is selected at each instance from alkyl (including C₁-C₄alkyl), alkenyl (including C₂-C₄alkenyl), alkynyl (including C₂-C₄alkynyl), haloalkyl (including C₁-C₄haloalkyl), —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅ wherein the optional substituent is selected such that a stable compound results;
and wherein all other variables are as defined in embodiment 1.
78. The compound of embodiment 77, wherein the compound is selected from:

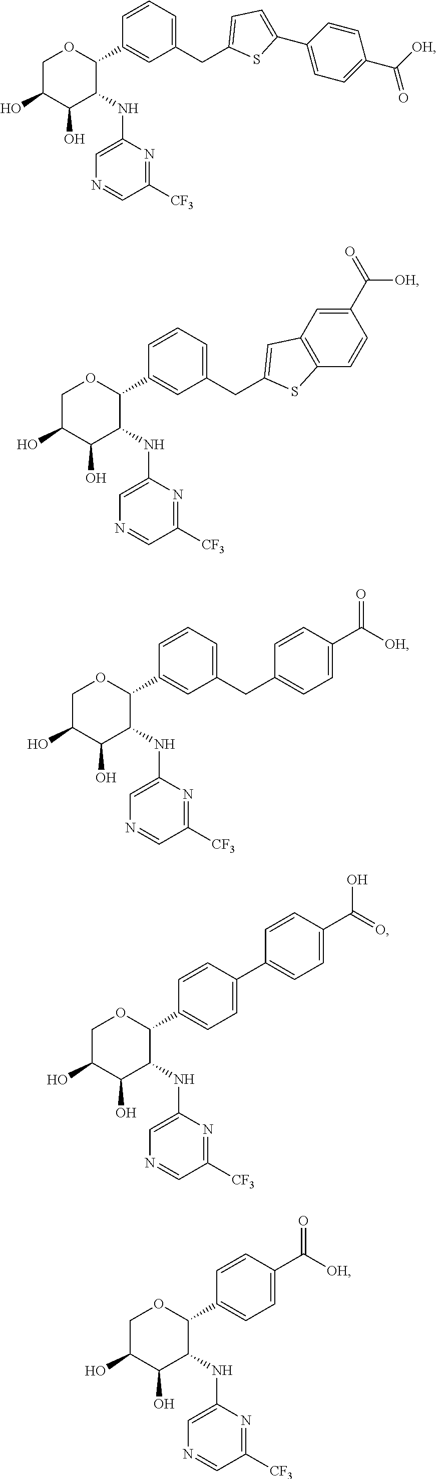 [SMILES: O=C(O)c1ccc(-c2ccc(Cc3cccc([C@H]4OC[C@H](O)[C@H](O)[C@H]4Nc4cncc(C(F)(F)F)n4)c3)s2)cc1 ; O=C(O)c1ccc2sc(Cc3cccc([C@H]4OC[C@H](O)[C@H](O)[C@H]4Nc4cncc(C(F)(F)F)n4)c3)cc2c1 ; O=C(O)c1ccc(Cc2cccc([C@H]3OC[C@H](O)[C@H](O)[C@H]3Nc3cncc(C(F)(F)F)n3)c2)cc1 ; O=C(O)c1ccc(-c2ccc([C@H]3OC[C@H](O)[C@H](O)[C@H]3Nc3cncc(C(F)(F)F)n3)cc2)cc1 ; O=C(O)c1ccc([C@H]2OC[C@H](O)[C@H](O)[C@H]2Nc2cncc(C(F)(F)F)n2)cc1]

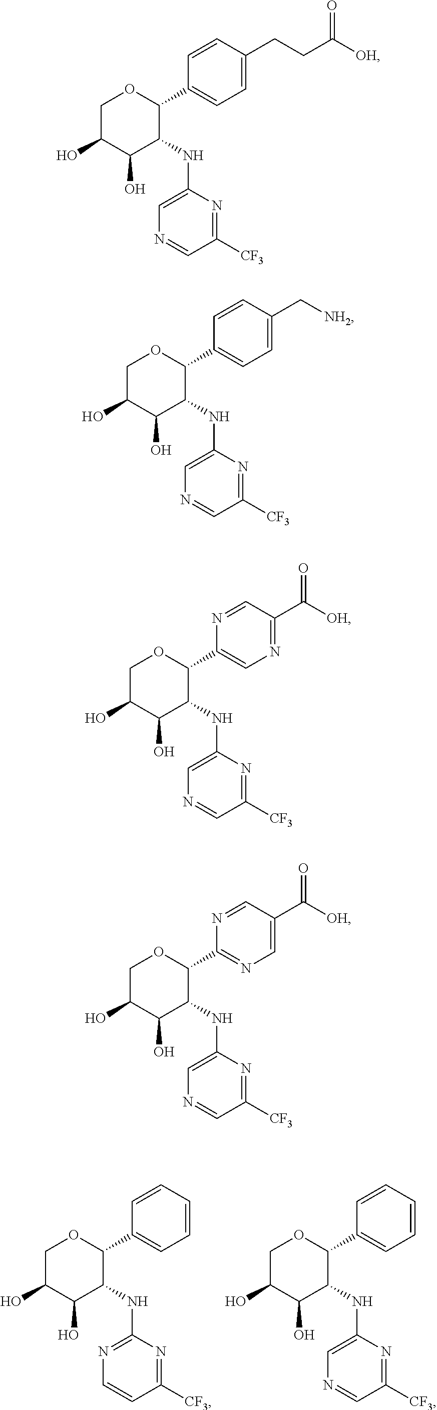

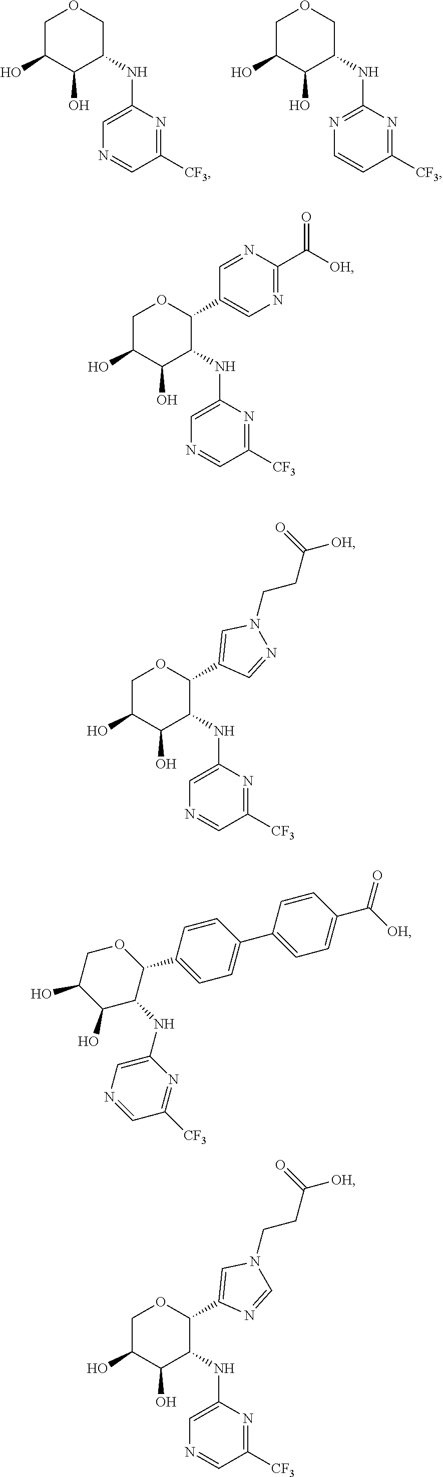 [SMILES: O[C@@H]1[C@@H](Nc2cncc(C(F)(F)F)n2)COC[C@@H]1O ; O[C@@H]1[C@@H](Nc2nccc(C(F)(F)F)n2)COC[C@@H]1O ; O=C(O)c1ncc([C@H]2OC[C@H](O)[C@H](O)[C@H]2Nc2cncc(C(F)(F)F)n2)cn1 ; O=C(O)CCn1cc([C@H]2OC[C@H](O)[C@H](O)[C@H]2Nc2cncc(C(F)(F)F)n2)cn1 ; O=C(O)c1ccc(-c2ccc([C@H]3OC[C@H](O)[C@H](O)[C@H]3Nc3cncc(C(F)(F)F)n3)cc2)cc1 ; O=C(O)CCn1cnc([C@H]2OC[C@H](O)[C@H](O)[C@H]2Nc2cncc(C(F)(F)F)n2)c1]

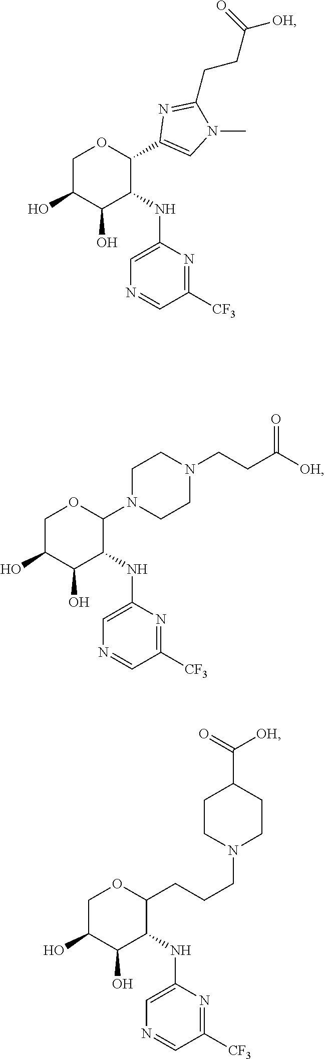 [SMILES: Cn1cc([C@H]2OC[C@H](O)[C@H](O)[C@H]2Nc2cncc(C(F)(F)F)n2)nc1CCC(=O)O ; O=C(O)CCN1CCN(C2OC[C@H](O)[C@H](O)[C@H]2Nc2cncc(C(F)(F)F)n2)CC1 ; O=C(O)C1CCN(CCCC2OC[C@H](O)[C@H](O)[C@H]2Nc2cncc(C(F)(F)F)n2)CC1]

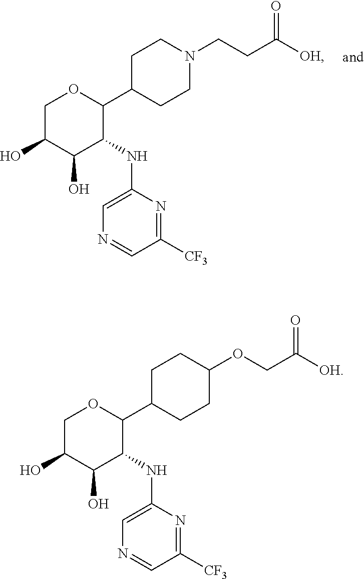 [SMILES: O=C(O)CCN1CCC(C2OC[C@H](O)[C@H](O)[C@H]2Nc2cncc(C(F)(F)F)n2)CC1 ; O=C(O)COC1CCC(C2OC[C@H](O)[C@H](O)[C@H]2Nc2cncc(C(F)(F)F)n2)CC1]

79. The compound of embodiment 77, wherein the compound is selected from:

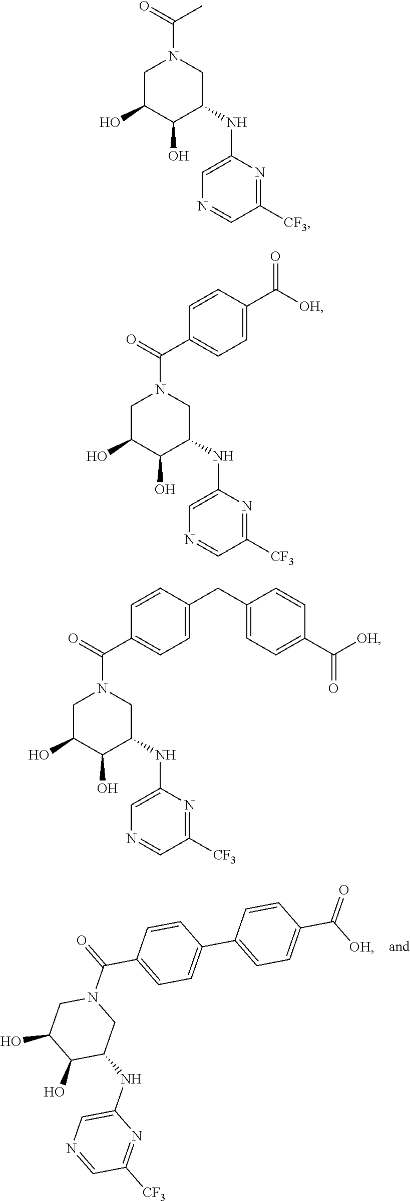

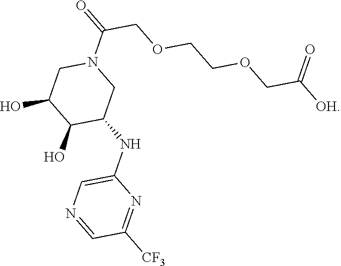 [SMILES: O=C(O)COCCOCC(=O)N1C[C@H](Nc2cncc(C(F)(F)F)n2)[C@@H](O)[C@@H](O)C1]

80. The compound of embodiment 77, wherein the compound is selected from:

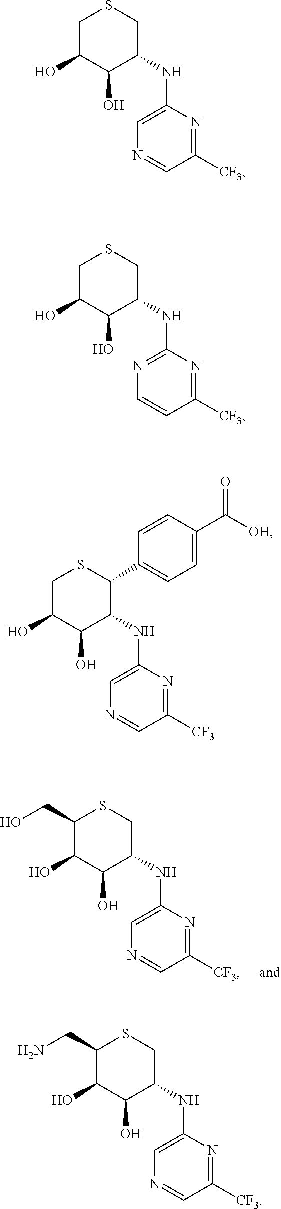 [SMILES: O[C@@H]1[C@@H](Nc2cncc(C(F)(F)F)n2)CSC[C@@H]1O ; O[C@@H]1[C@@H](Nc2nccc(C(F)(F)F)n2)CSC[C@@H]1O ; O=C(O)c1ccc([C@H]2SC[C@H](O)[C@H](O)[C@H]2Nc2cncc(C(F)(F)F)n2)cc1 ; OC[C@H]1SC[C@H](Nc2cncc(C(F)(F)F)n2)[C@@H](O)[C@H]1O ; NC[C@H]1SC[C@H](Nc2cncc(C(F)(F)F)n2)[C@@H](O)[C@H]1O]

81. The compound of embodiment 77, wherein the compound is selected from:

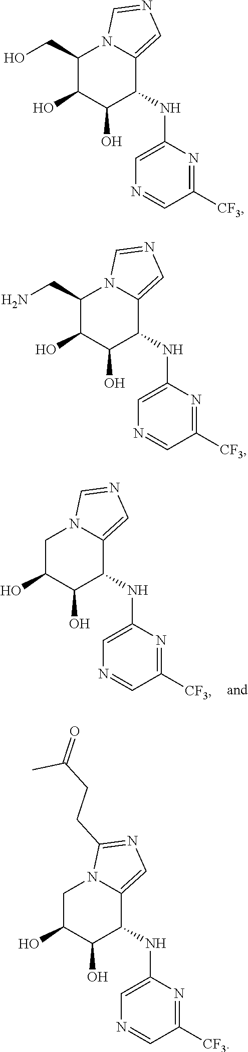

82. The compound of embodiment 77, wherein the compound is selected from:

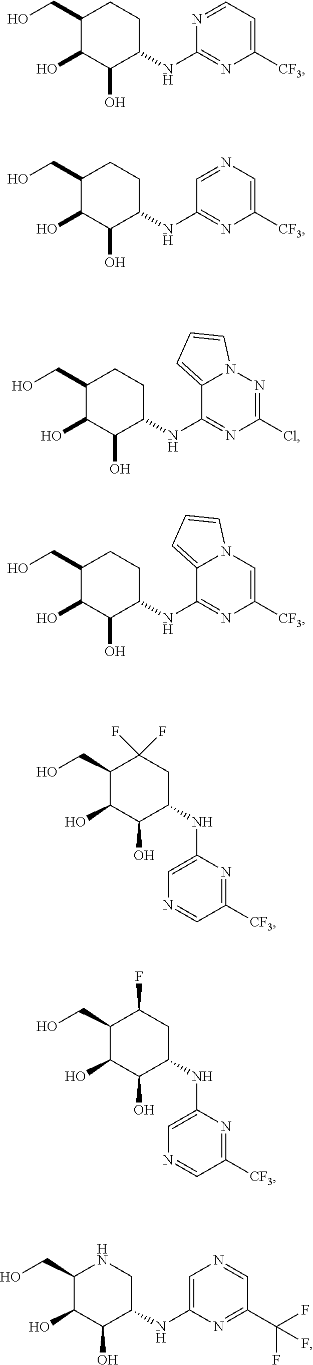

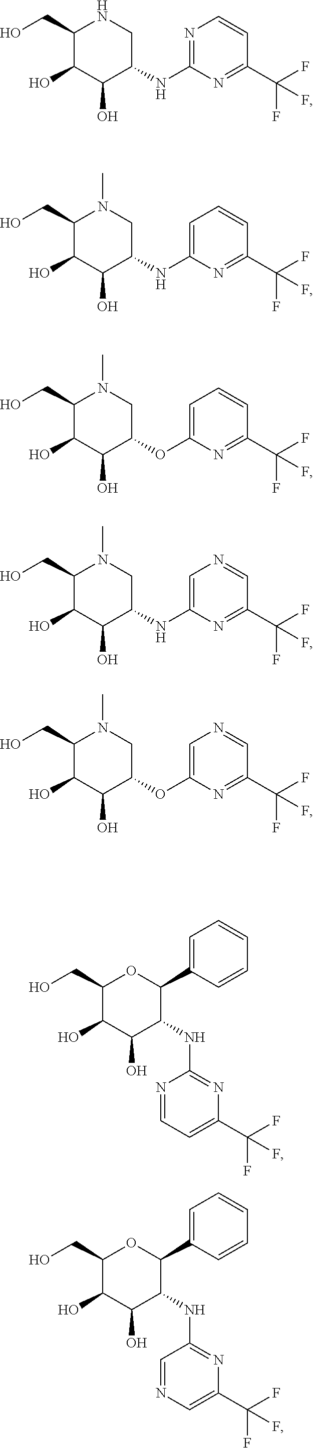

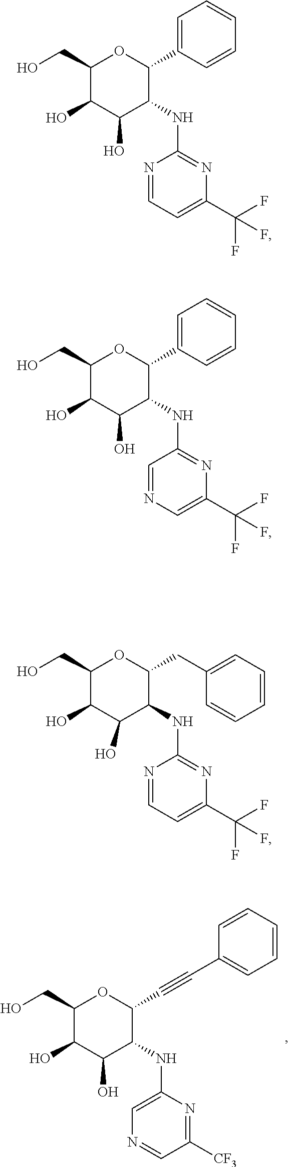 [SMILES: OC[C@H]1O[C@H](c2ccccc2)[C@H](Nc2nccc(C(F)(F)F)n2)[C@@H](O)[C@H]1O ; OC[C@H]1O[C@H](c2ccccc2)[C@H](Nc2cncc(C(F)(F)F)n2)[C@@H](O)[C@H]1O ; OC[C@H]1O[C@H](Cc2ccccc2)[C@@H](Nc2nccc(C(F)(F)F)n2)[C@@H](O)[C@H]1O ; OC[C@H]1O[C@H](C#Cc2ccccc2)[C@H](Nc2cncc(C(F)(F)F)n2)[C@@H](O)[C@H]1O]

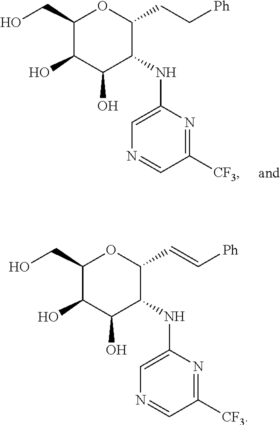 [SMILES: OC[C@H]1O[C@H](CCc2ccccc2)[C@H](Nc2cncc(C(F)(F)F)n2)[C@@H](O)[C@H]1O ; OC[C@H]1O[C@H](/C=C/c2ccccc2)[C@H](Nc2cncc(C(F)(F)F)n2)[C@@H](O)[C@H]1O]

1. An ASGPR-binding extracellular protein degrader compound of the formula:

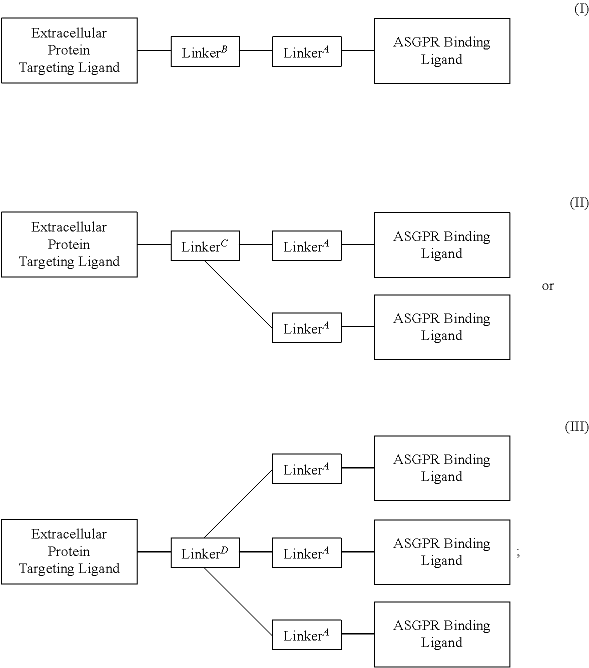

or a pharmaceutically acceptable salt thereof;
wherein the ASGPR Binding Ligand is selected from:

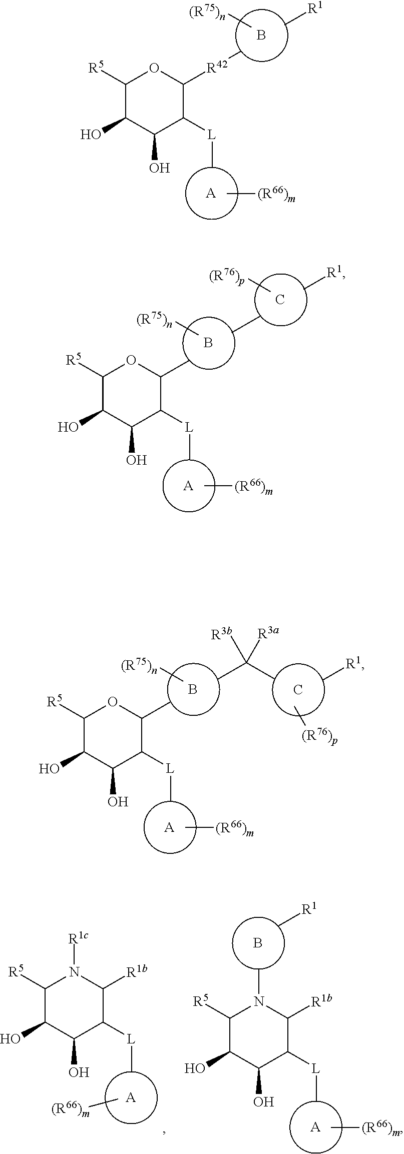

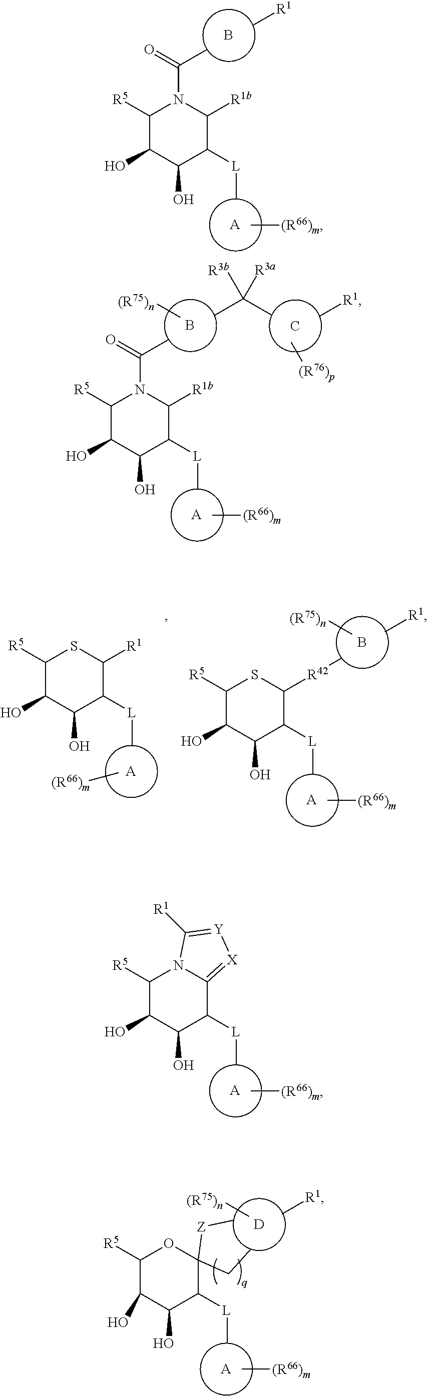

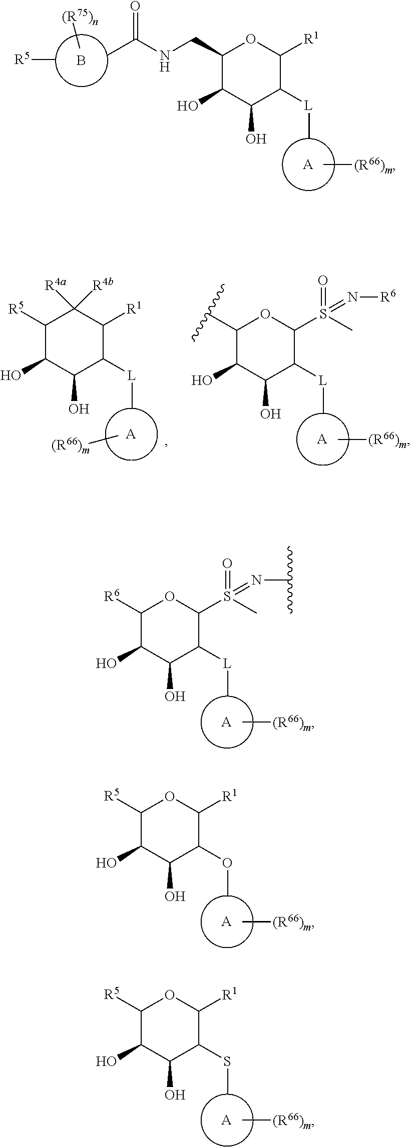

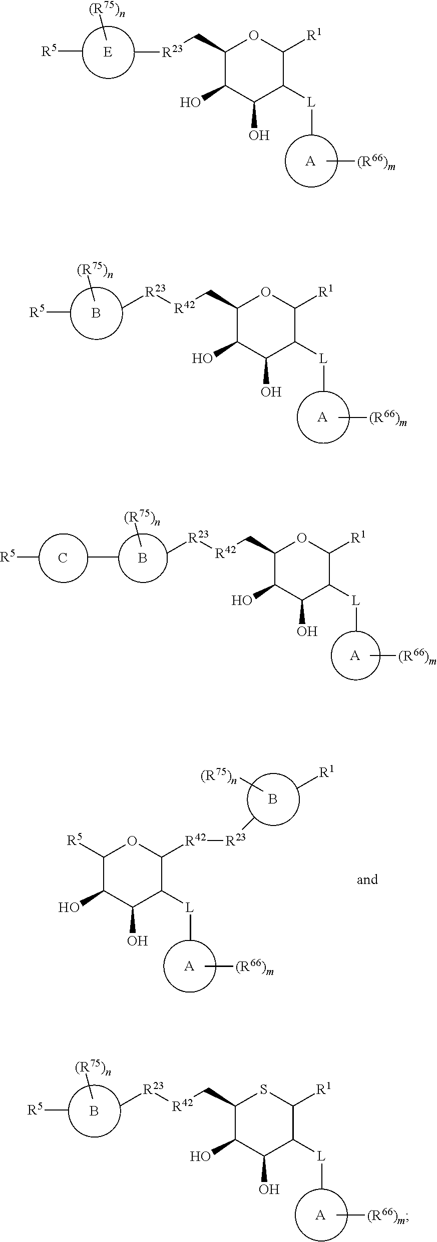

R¹, R^{1b}, and R⁵ are independently selected from hydrogen, C₀-C₆alkyl-cyano, alkyl, alkenyl, alkynyl, haloalkyl, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, and C₀-C₆alkylN₃, each of which except hydrogen, F, Cl, and Br is optionally substituted with 1, 2, or 3 substituents independently selected at each occurrence from R⁹⁹;
R^{1c} is selected from hydrogen, alkyl, alkenyl, alkynyl, haloalkyl, —C(O)R³, —S(O)R³, —C(S)R³, and —S(O)₂R³;
wherein one of R¹, R^{1b}, R^{1c}, and R⁵ is replaced with a bond to Linker^{A};
L is selected from

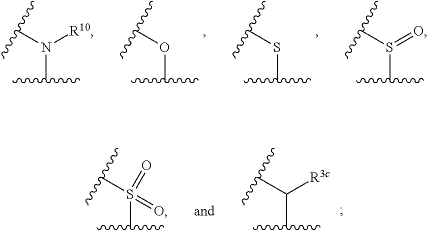

R³, R^{3a}, R^{3b}, R^{3c}, and R^{3d} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R^{4a} is selected from hydrogen, alkyl, haloalkyl, and halogen;
R^{4b} is selected from hydrogen, alkyl, haloalkyl, halogen, C₀-C₆alkyl-OR⁶, C₀-C₆alkyl-SR⁶, and C₀-C₆alkyl-NR⁶R⁷;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
R⁴² is selected from bond, C₁-C₄ alkyl, C₁-C₄ haloalkyl, C₂-C₄ alkenyl, and C₂-C₄ alkynyl;
R²³ is selected from bond,

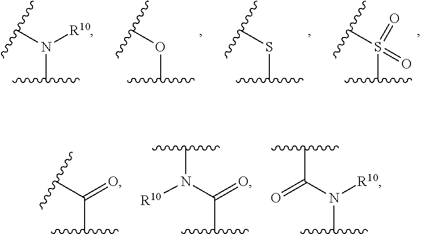

C₁-C₄ alkyl, C₁-C₄ haloalkyl, C₂-C₄ alkenyl, and C₂-C₄ alkynyl;
R⁶⁶ is independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰;
R⁷⁵ and R⁷⁶ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, or 3 substituents independently selected at each occurrence from R¹⁰²;
m and p are independently 0, 1, 2, or 3, as allowed by valence;
n is independently selected at each instance from 0, 1, 2, or 3;
q is 1, 2, or 3;
\textcircled{A} is heteroaryl;
\textcircled{B} is aryl, heterocycle, cycloalkyl, or heteroaryl;
\textcircled{C} is aryl, heterocycle, cycloalkyl, bicycle, or heteroaryl;
\textcircled{D} is aryl or heteroaryl;
\textcircled{E} is a bicycle or spirocycle;
X is CH, CR⁷⁵, or N;
Y is CH, CR⁷⁵, or N;
Z is selected from —O—, —NR⁶—, —S—, —S(O)—, —S(O)₂—, and —CR^{3a}R^{3b}—;
R⁹⁹, R¹⁰⁰, and R¹⁰² are independently selected at each instance from alkyl, alkenyl, alkynyl, haloalkyl, —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and -SFs;
Linker^{A} and Linker^{B} are:

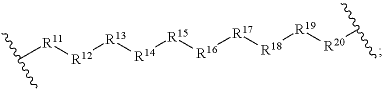

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, or 3 substituents independently selected from R²¹;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, heterocyclyl, —SR³, —C(O)OR³, —OR³,

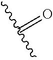 [SMILES: *=O]

and heterocycle;
Linker^{C} is:

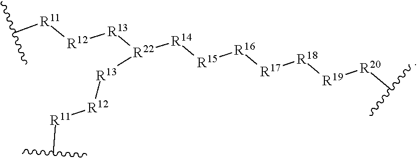

wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, or 3 substituents independently selected from R²¹;
Linker^{D} is selected from:

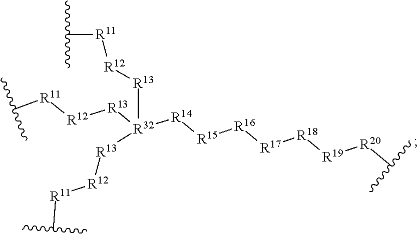

wherein:
R³² is independently at each occurrence selected from the group consisting of alkyl, N⁺X⁻, —C—, alkenyl, haloalkyl, aryl, heterocycle, and heteroaryl, each of which is optionally substituted with 1, 2, or 3 substituents independently selected from R²¹;
X⁻ is an anionic group Br⁻ or Cl⁻; and
Extracellular Protein Targeting Ligand is a Ligand that binds to an extracellular protein.
2. The compound of embodiment 1, wherein L is selected from

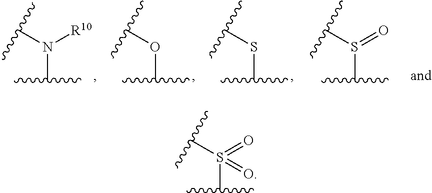

3. The compound of embodiment 1 or 2, wherein L is selected from

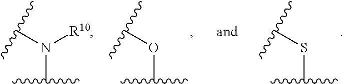

4. The compound of embodiment 1, wherein the ASPGR Binding Ligand is selected from:

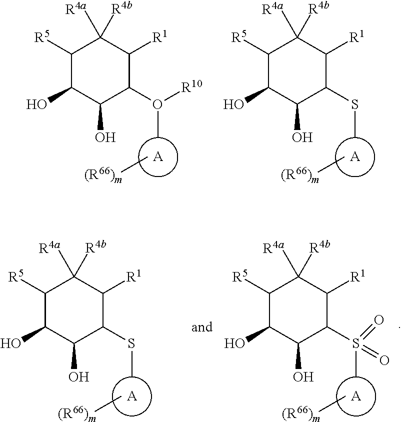

5. The compound of embodiment 4, wherein R^{4a} is hydrogen.
6. The compound of embodiment 4, wherein R^{4a} is F.
7. The compound of any one of embodiments 4-6, wherein R^{4b} is hydrogen.
8. The compound of any one of embodiments 4-6, wherein R^{4b} is F.
9. The compound of embodiment 1, wherein the ASPGR Binding Ligand is selected from:

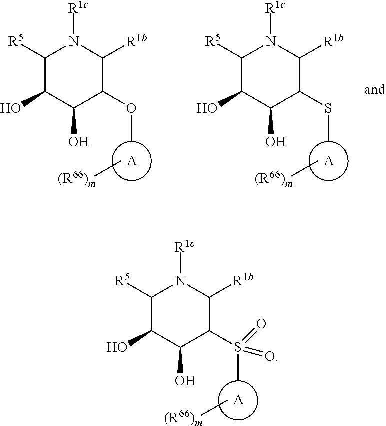

10. The compound of embodiment 1 or embodiment 9, wherein R^{1c} is a bond to Linker^{A}.
11. The compound of embodiment 1 or embodiment 9, wherein R^{1c} is hydrogen.
12. The compound of embodiment 1, wherein the ASPGR Binding Ligand is selected from:

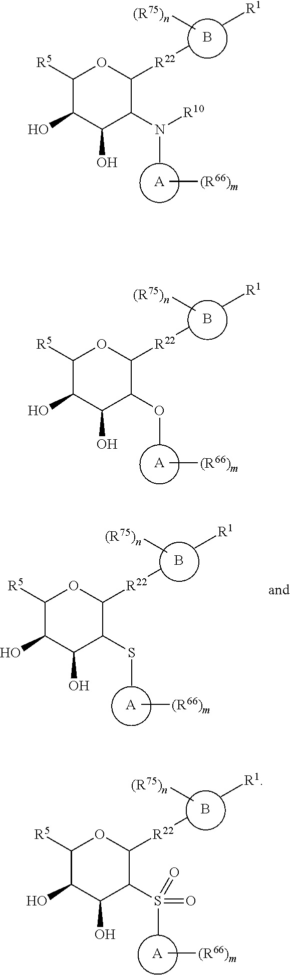

13. The compound of embodiment 1 or embodiment 12, wherein R⁴² is bond.
14. The compound of embodiment 1 or embodiment 12, wherein R⁴² is C₁-C₄ alkyl.
15. The compound of embodiment 1 or embodiment 12, wherein R⁴² is C₁-C₄ haloalkyl.
16. The compound of any one of embodiments 12-15, wherein \textcircled{B} is phenyl.
17. The compound of embodiment 1, wherein the ASPGR Binding Ligand is:

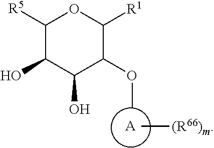

18. The compound of embodiment 1, wherein the ASGPR Binding Ligand is selected from:

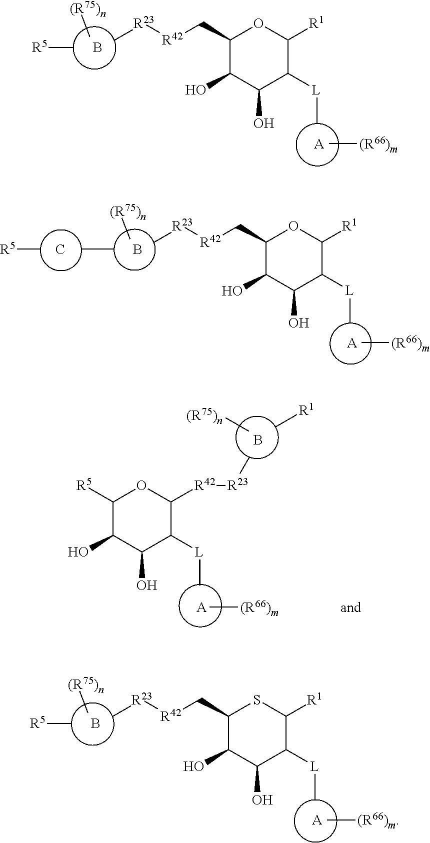

19. The compound of embodiment 18, wherein \textcircled{B} is heteroaryl.
20. The compound of embodiment 19, wherein \textcircled{B} is selected from

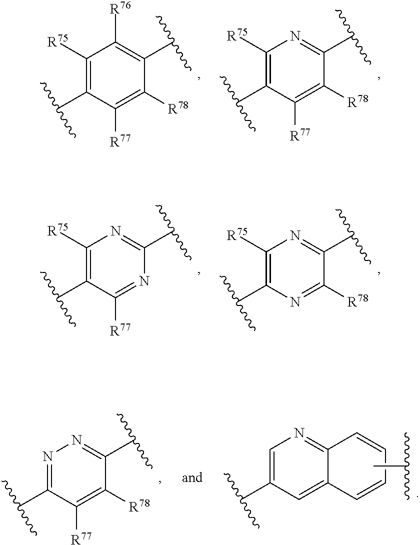

wherein:
R⁷⁷ and R⁷⁸ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, or 3 substituents independently selected at each occurrence from R¹⁰².
21. The compound of any one of embodiments 18-20, wherein R⁴² is C₁-C₄ alkyl.
22. The compound of any one of embodiments 18-21, wherein R²³ is —O—.
23. The compound of any one of embodiments 18-21, wherein R²³ is —NR¹⁰—.
24. The compound of any one of embodiments 1-23, wherein

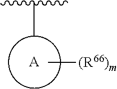

is selected from:

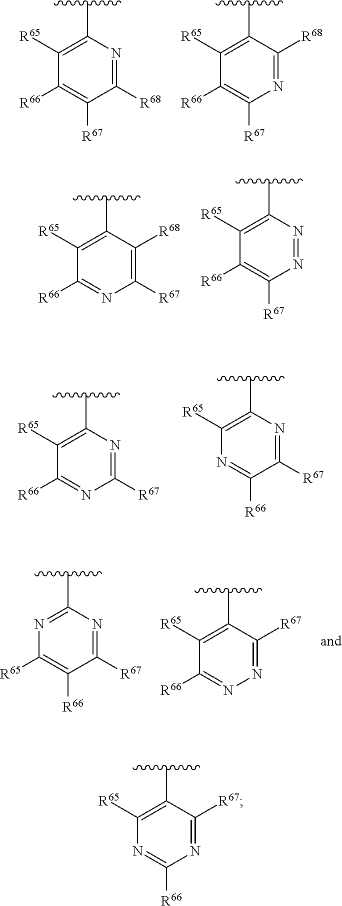

R⁶⁵, R⁶⁷, and R⁶⁸ are independently selected from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰.
25. The compound of embodiment 24, wherein R⁶⁵ is hydrogen.
26. The compound of embodiment 24, wherein R⁶⁵ is halogen.
27. The compound of embodiment 26, wherein R⁶⁵ is F or Cl.
28. The compound of embodiment 24, wherein R⁶⁵ is C₁-C₄alkyl.
29. The compound of embodiment 24, wherein R⁶⁵ is C₁-C₄haloalkyl.
30. The compound of embodiment 29, wherein R⁶⁵ is CF₃.
31. The compound of any one of embodiments 1-30, wherein R⁶⁶ is hydrogen.
32. The compound of any one of embodiments 1-30, wherein R⁶⁶ is halogen.
33. The compound of any one of embodiments 1-30, wherein R⁶⁶ is F or Cl.
34. The compound of any one of embodiments 1-30, wherein R⁶⁶ is C₁-C₄alkyl.
35. The compound of any one of embodiments 1-30, wherein R⁶⁶ is C₁-C₄haloalkyl.
36. The compound of any one of embodiments 1-30, wherein R⁶⁶ is CF₃.
37. The compound of any one of embodiments 24-36, wherein R⁶⁷ is hydrogen.
38. The compound of any one of embodiments 24-36, wherein R⁶⁷ is halogen.
39. The compound of any one of embodiments 24-36, wherein R⁶⁷ is F or Cl.
40. The compound of any one of embodiments 24-36, wherein R⁶⁷ is C₁-C₄alkyl.
41. The compound of any one of embodiments 24-36, wherein R⁶⁷ is C₁-C₄haloalkyl.
42. The compound of any one of embodiments 24-36, wherein R⁶⁷ is CF₃
43. The compound of any one of embodiments 1-23, wherein

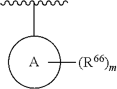

is selected from:

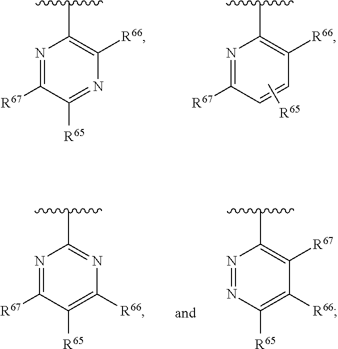

44. The compound of any one of embodiments 1-23, wherein

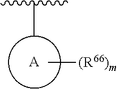

is

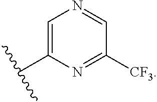 [SMILES: *c1cncc(C(F)(F)F)n1]

45. The compound of any one of embodiments 1-23, wherein

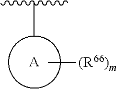

is

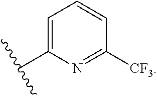 [SMILES: *c1cccc(C(F)(F)F)n1]

46. The compound of any one of embodiments 1-23, wherein

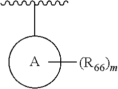

is selected from:

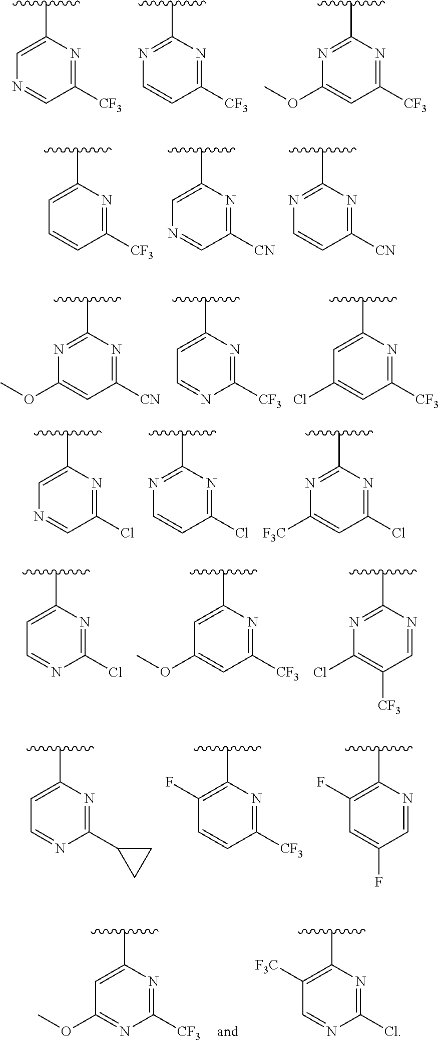

47. The compound of embodiment 1, wherein the ASGPR Binding Ligand is selected from:

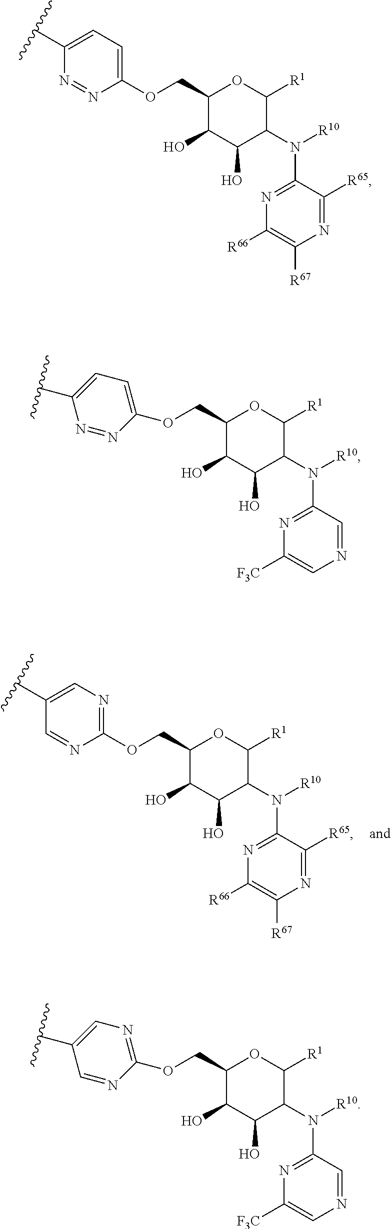

48. The compound of embodiment 1, wherein the ASPGR Binding Ligand is selected from:

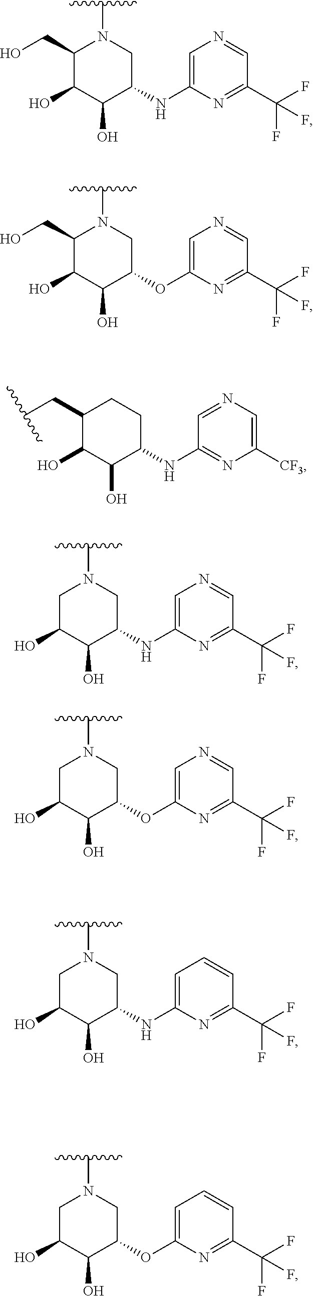

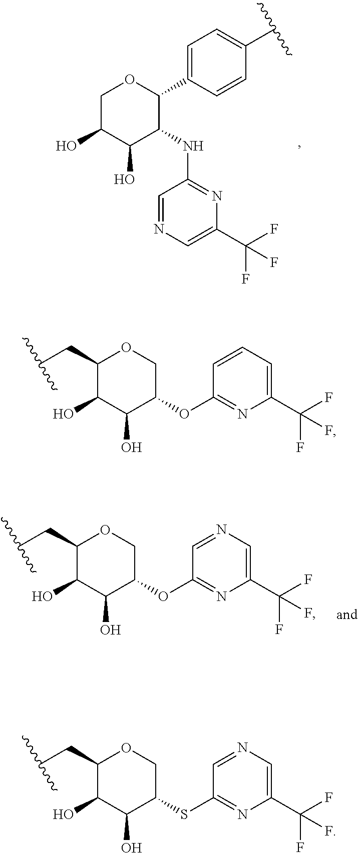 [SMILES: *c1ccc([C@H]2OC[C@H](O)[C@H](O)[C@H]2Nc2cncc(C(F)(F)F)n2)cc1 ; *C[C@H]1OC[C@H](Oc2cccc(C(F)(F)F)n2)[C@@H](O)[C@H]1O ; *C[C@H]1OC[C@H](Oc2cncc(C(F)(F)F)n2)[C@@H](O)[C@H]1O ; *C[C@H]1OC[C@H](Sc2cncc(C(F)(F)F)n2)[C@@H](O)[C@H]1O]

49. The compound of embodiment 1, wherein the ASPGR Binding Ligand is a compound selected from:

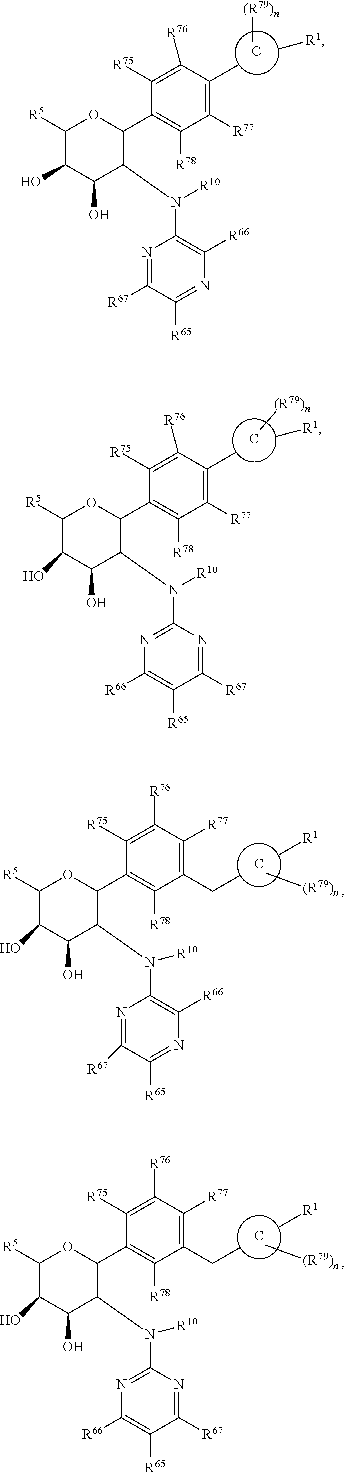

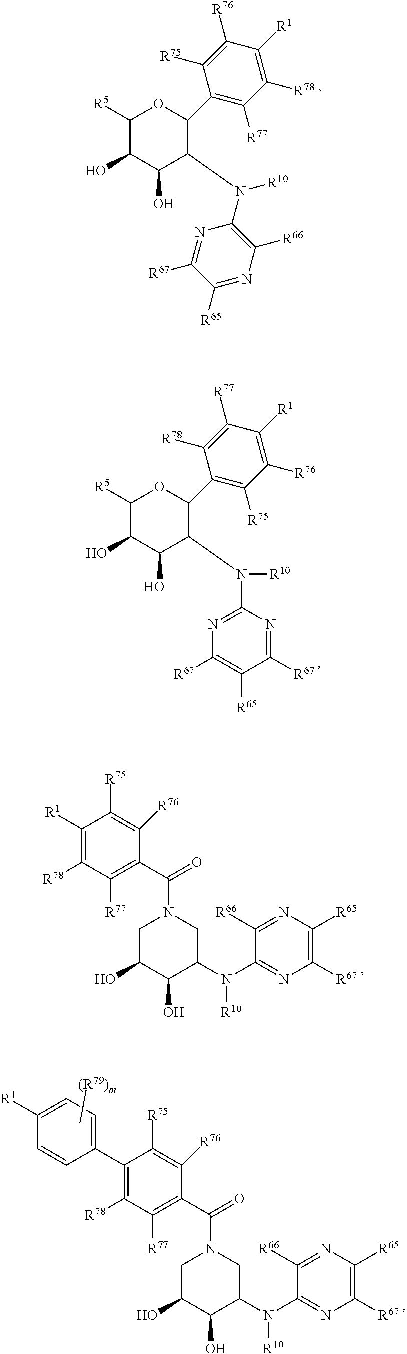

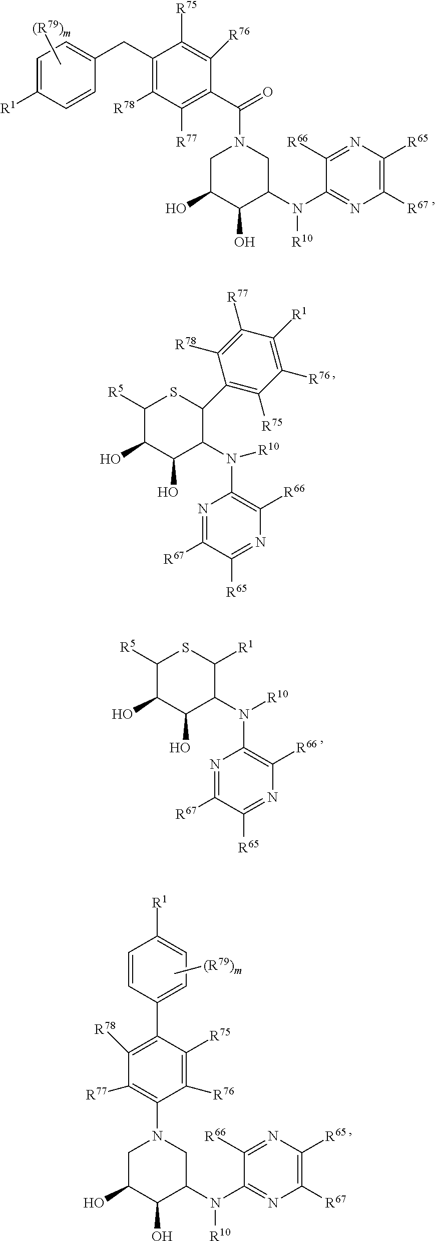

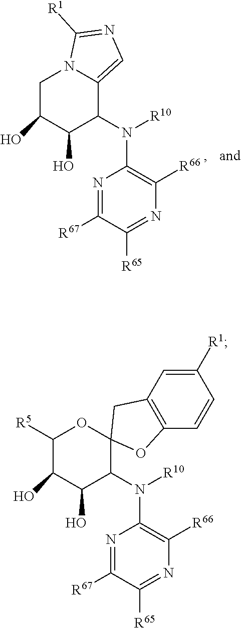

wherein R⁶⁵ and R⁶⁷ are independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰; and
R⁷⁷, R⁷⁸, and R⁷⁹ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, or 3 substituents independently selected at each occurrence from R¹⁰².
50. The compound of embodiment 1, wherein the compound is of Formula

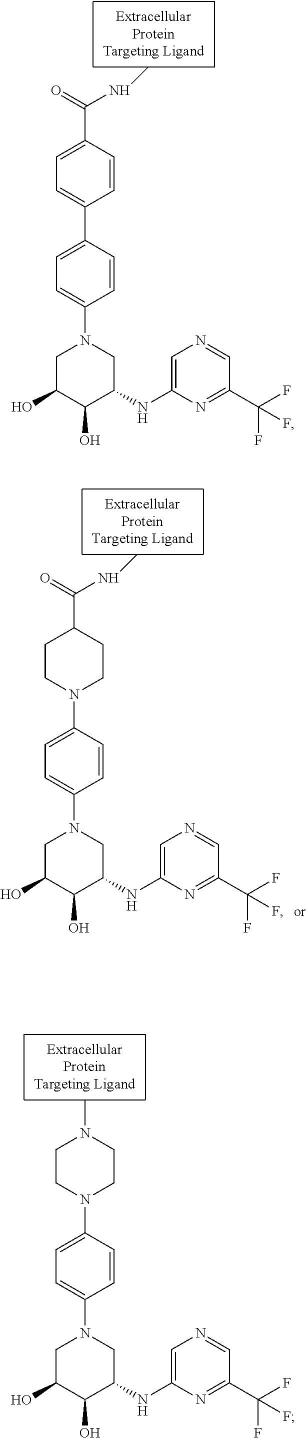 [SMILES: CNC(=O)c1ccc(-c2ccc(N3C[C@H](Nc4cncc(C(F)(F)F)n4)[C@@H](O)[C@@H](O)C3)cc2)cc1 ; CNC(=O)C1CCN(c2ccc(N3C[C@H](Nc4cncc(C(F)(F)F)n4)[C@@H](O)[C@@H](O)C3)cc2)CC1 ; CN1CCN(c2ccc(N3C[C@H](Nc4cncc(C(F)(F)F)n4)[C@@H](O)[C@@H](O)C3)cc2)CC1]

or a pharmaceutically acceptable salt thereof.
51. The compound of embodiment 1, wherein the compound is of Formula:

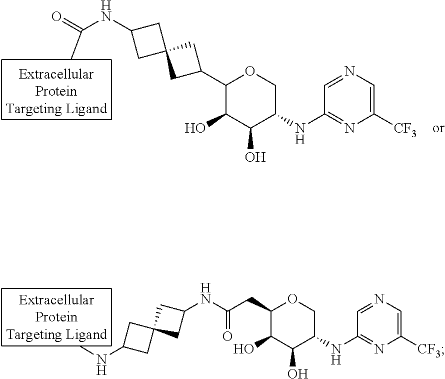

or a pharmaceutically acceptable salt thereof.
52. The compound of any one of embodiments 1-51, wherein the Extracellular Protein Targeting Ligand is a means for binding the targeted extracellular protein that creates or exacerbates a disease.
53. The compound of any one of embodiments 1-52, wherein the Extracellular Protein Targeting Ligand binds IgG.
54. The compound of embodiment 53, wherein the IgG targeting Extracellular Protein Targeting Ligand is Fc-BP-2.
55. The compound of embodiment 53, wherein the IgG targeting Extracellular Protein Targeting Ligand is Fc-III.
56. The compound of any one of embodiments 1-52, wherein the Extracellular Protein Targeting Ligand binds IgM.
57. The compound of any one of embodiments 1-52, wherein the Extracellular Protein Targeting Ligand targets IgE.
58. The compound of any one of embodiments 1-52, wherein the Extracellular Protein Targeting Ligand targets IgA.
59. The compound of any one of embodiments 1-52, wherein the Extracellular Protein Targeting Ligand targets TNF-α.
60. The compound of any one of embodiments 1-52, wherein the Extracellular Protein Targeting Ligand targets IL-1b.
61. The compound of any one of embodiments 1-52, wherein the Extracellular Protein Targeting Ligand targets IL-2.
62. The compound of any one of embodiments 1-52, wherein the Extracellular Protein Targeting Ligand targets TL-6.
63. The compound of any one of embodiments 1-52, wherein the Extracellular Protein Targeting Ligand targets IFN-γ.
64. The compound of any one of embodiments 1-52, wherein the Extracellular Protein Targeting Ligand targets VEGF.
65. The compound of any one of embodiments 1-52, wherein the Extracellular Protein Targeting Ligand targets TGF-b1.
66. The compound of any one of embodiments 1-52, wherein the Extracellular Protein Targeting Ligand targets PCSK-9.
67. The compound of any one of embodiments 1-52, wherein the Extracellular Protein Targeting Ligand targets IL-17.
68. The compound of embodiment 67, wherein the IL-17 Targeting Ligand is of the formula:

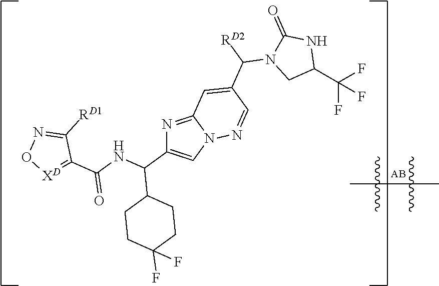

wherein,
X^{D} is CH or N;
R^{D1} is —CH₃, —CH₂F, —CHF₂, —CF₃, —CH₂CH₃, —CH₂CF₃, —CH(CH₃)₂, CH₂CHF₂, CH₂CH₂F, —CF(CH₃)₂, CF₂CH₃, —OCH₃,

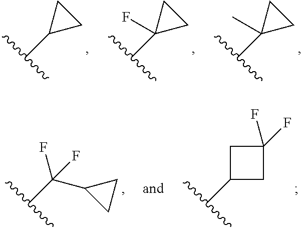

R^{D2} is —H or —CH₂OCH₃;
AB is the attachment point to Linker^{B}, Linker^{C}, or Linker^{D}.
69. The compound of any one of embodiments 1-52, wherein the Extracellular Protein Targeting Ligand targets GP120.
70. The compound of any one of embodiments 1-52, wherein the Extracellular Protein Targeting Ligand targets a protein selected from Serum amyloid P component, amyloid precursor protein, C reactive protein (CRP), an N-methyl-D-aspartate (NMDA) receptor, a-synuclein, IAPP, and transthyretin.
71. The compound of any one of embodiments 1-52, wherein the Extracellular Protein Targeting Ligand targets autoantibodies to the R 1 adrenergic receptor.
72. The compound of any one of embodiments 1-71, wherein Linker^{A} is:

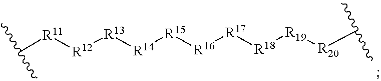

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, each of which is optionally substituted with 1 substituent independently selected from R²¹.
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.
73. The compound of any one of embodiments 1-72, wherein R¹⁶, R¹⁷, R¹⁸, R¹⁹, and R²⁰ are bond.
74. The compound of any one of embodiments 1-73, wherein R¹⁵ is bond.
75. The compound of any one of embodiments 1-74, wherein R¹⁴ is bond.
76. The compound of any one of embodiments 1-75, wherein R¹³ is bond
77. The compound of any one of embodiments 1-76, wherein R¹² is bond
78. The compound of any one of embodiments 1-77, wherein R¹¹ is bond
79. The compound of any one of embodiments 1-72, wherein nine of R¹¹, R¹², R¹³, R¹⁴, R¹⁵, R¹⁶, R¹⁷, R¹⁸, R¹⁹, and R²⁰ are bond.
80. The compound of any one of embodiments 1-72, wherein eight of R¹¹, R¹², R¹³, R¹⁴, R¹⁵, R¹⁶, R¹⁷, R¹⁸, R¹⁹, and R²⁰ are bond.
81. The compound of any one of embodiments 1-72, wherein seven of R¹¹, R¹², R¹³, R¹⁴, R¹⁵, R¹⁶, R¹⁷, R¹⁸, R¹⁹, and R²⁰ are bond.
82. The compound of any one of embodiments 1-72, wherein six of R¹¹, R¹², R¹³, R¹⁴, R¹⁵, R¹⁶, R¹⁷, R¹⁸, R¹⁹, and R²⁰ are bond.
83. The compound of any one of embodiments 1-72, wherein five of R¹¹, R¹², R¹³, R¹⁴, R¹⁵, R¹⁶, R¹⁷, R¹⁸, R¹⁹, and R²⁰ are bond.
84. The compound of any one of embodiments 1-72, wherein four of R¹¹, R¹², R¹³, R¹⁴, R¹⁵, R¹⁶, R¹⁷, R¹⁸, R¹⁹, and R²⁰ are bond.
85. The compound of any one of embodiments 1-72, wherein three of R¹¹, R¹², R¹³, R¹⁴, R¹⁵, R¹⁶, R¹⁷, R¹⁸, R¹⁹, and R²⁰ are bond.
86. The compound of any one of embodiments 1-72, wherein two of R¹¹, R¹², R¹³, R¹⁴, R¹⁵, R¹⁶, R¹⁷, R¹⁸, R¹⁹, and R²⁰ are bond.
87. The compound of any one of embodiments 1-72, wherein one of R¹¹, R¹², R¹³, R¹⁴, R¹⁵, R¹⁶, R¹⁷, R¹⁸, R¹⁹, and R²⁰ is bond.
88. The compound of any one of embodiments 1-72, wherein Linker^{A} is

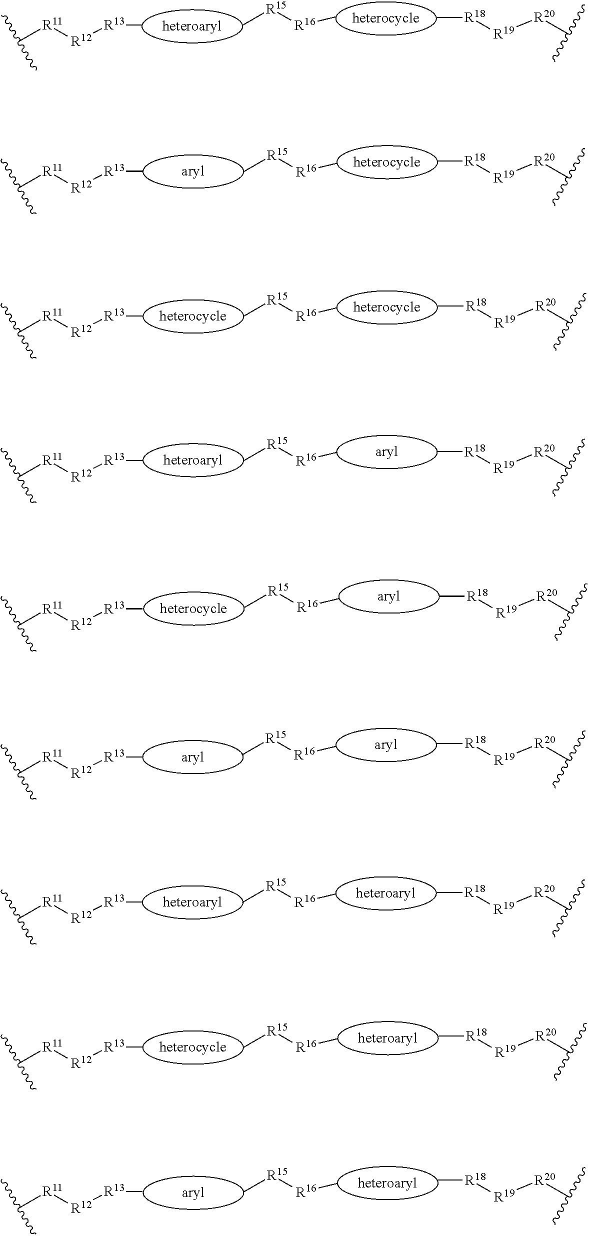

wherein each heteroaryl, heterocycle, and aryl can optionally be substituted with 1, 2, or 3 of any combination of halogen, alkyl, haloalkyl, aryl, heteroaryl, heterocycle, or cycloalkyl.
89. The compound of embodiment 88, wherein R¹¹, R¹², R¹³, R, R¹⁶, R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.
90. The compound of any one of embodiments 1-72, wherein Linker^{A} is selected from:

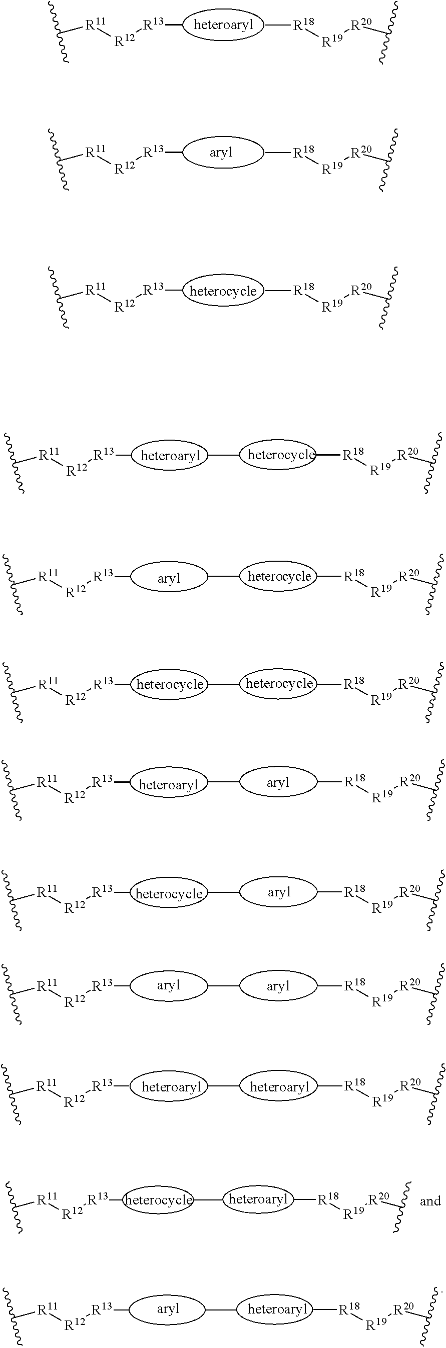

91. The compound of embodiment 90, wherein R¹¹, R¹², R¹³, R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)—, alkenyl, alkynyl, haloalkyl, alkoxy, aryl, heterocycle, heteroaryl, and —CH₂CH₂—[O—(CH₂)₂]ₙ—O—.
92. The compound of any one of embodiments 88-91, wherein R¹¹ is bond.
93. The compound of any one of embodiments 88-91, wherein R¹¹ is CH₂.
94. The compound of any one of embodiments 88-91, wherein R¹¹ is —O—.
95. The compound of any one of embodiments 88-91, wherein R¹¹ is —C(O)NR⁶—.
96. The compound of any one of embodiments 88-91, wherein R¹¹ is —C(O)O—.
97. The compound of any one of embodiments 88-96, wherein R²⁰ is bond.
98. The compound of any one of embodiments 88-96, wherein R²⁰ is CH₂.
99. The compound of any one of embodiments 88-96, wherein R²⁰ is —O—.
100. The compound of any one of embodiments 88-96, wherein R²⁰ is —C(O)NR⁶—.
101. The compound of any one of embodiments 88-96, wherein R²⁰ is —C(O)O—.
102. The compound of any one of embodiments 1-72, wherein Linker^{A} is selected from:

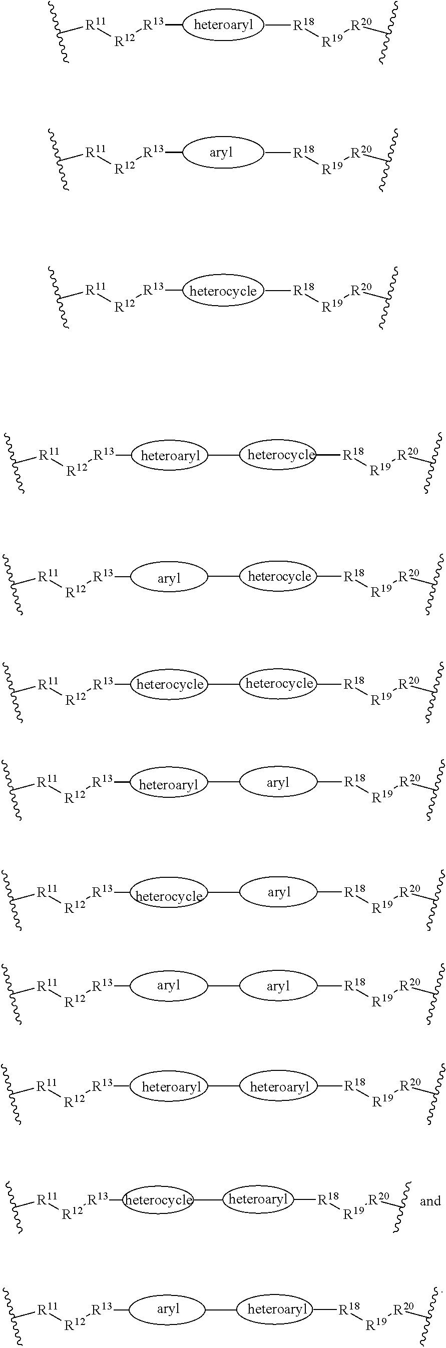

103. The compound of any one of embodiments 88-102, wherein R¹² is bond.
104. The compound of any one of embodiments 88-102, wherein R¹² is CH₂.
105. The compound of any one of embodiments 88-102, wherein R¹² is —O—.
106. The compound of any one of embodiments 88-102, wherein R¹² is —C(O)NR⁶—.
107. The compound of any one of embodiments 88-102, wherein R¹² is —C(O)O—.
108. The compound of any one of embodiments 88-107, wherein R¹⁹ is bond.
109. The compound of any one of embodiments 88-107, wherein R¹⁹ is CH₂.
110. The compound of any one of embodiments 88-107, wherein R¹⁹ is —O—.
111. The compound of any one of embodiments 88-107, wherein R¹⁹ is —C(O)NR⁶—.
112. The compound of any one of embodiments 88-107, wherein R¹⁹ is —C(O)O—.
113. The compound of any one of embodiments 1-72 wherein Linker^{A} is selected from:

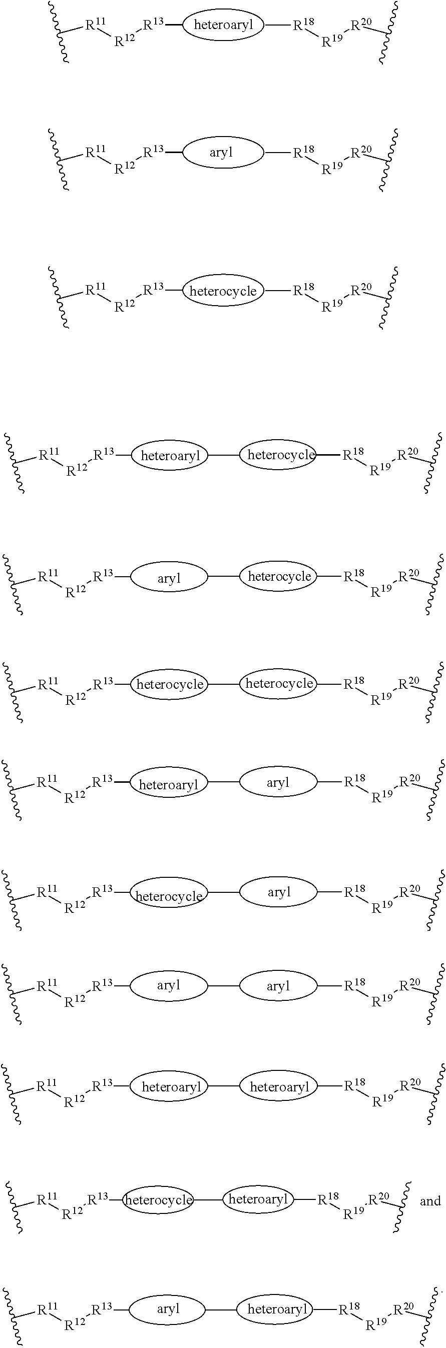

114. The compound of any one of embodiments 88-113, wherein R¹³ is bond.
115. The compound of any one of embodiments 88-113, wherein R¹³ is CH₂.
116. The compound of any one of embodiments 88-113, wherein R¹³ is —O—.
117. The compound of any one of embodiments 88-113, wherein R¹³ is —C(O)NR⁶—.
118. The compound of any one of embodiments 88-113, wherein R¹³ is —C(O)O—.
119. The compound of any one of embodiments 88-118, wherein R¹⁸ is bond.
120. The compound of any one of embodiments 88-118, wherein R¹⁸ is CH₂.
121. The compound of any one of embodiments 88-118, wherein R¹⁸ is —O—.
122. The compound of any one of embodiments 88-118, wherein R¹⁸ is —C(O)NR⁶—.
123. The compound of any one of embodiments 88-118, wherein R¹⁸ is —C(O)O—.
124. The compound of any one of embodiments 1-123, wherein aryl is phenyl.
125. The compound of any one of embodiments 72-124, wherein heteroaryl is selected from

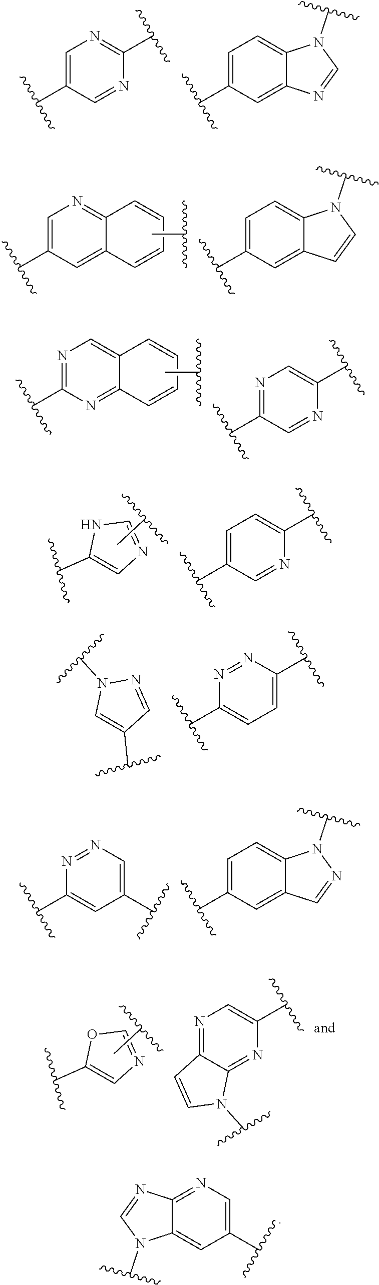

126. The compound of embodiment 125, wherein heteroaryl is selected from

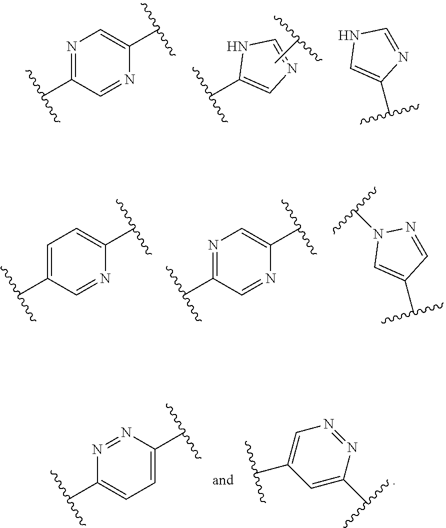

127. The compound of any one of embodiments 72-126, wherein heterocycle is selected from

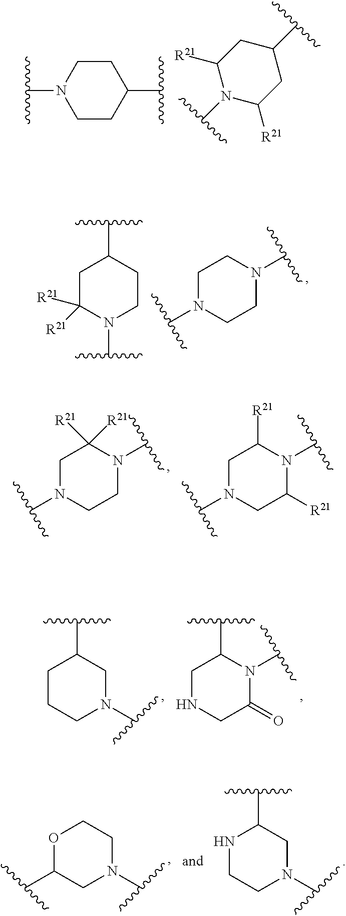

128. The compound of any one of embodiments 1-72, wherein Linker^{A} is selected from

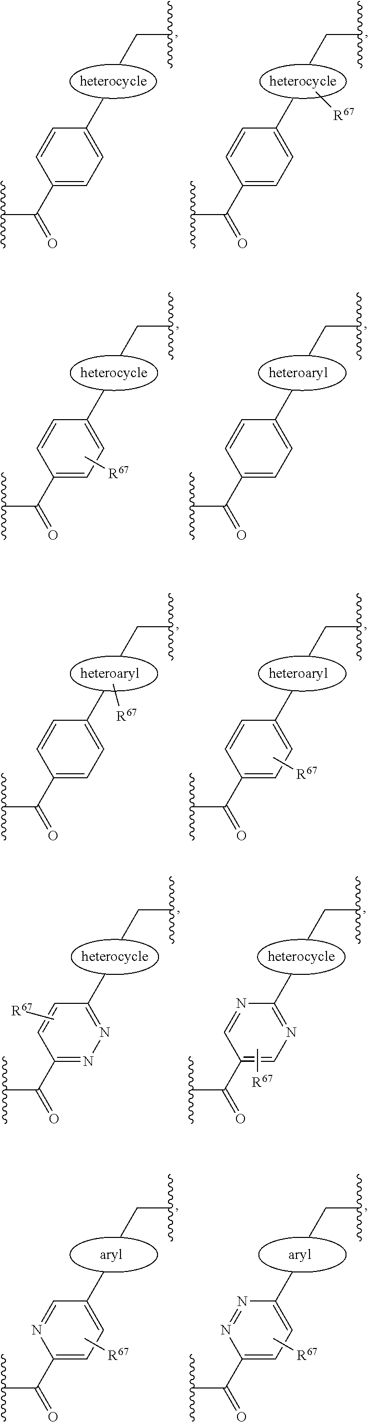

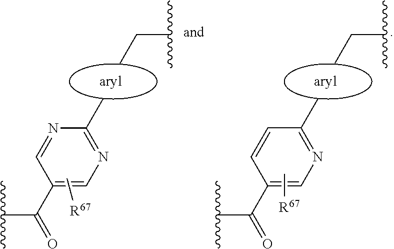

129. The compound of any one of embodiments 1-72, wherein Linker^{A} is selected from

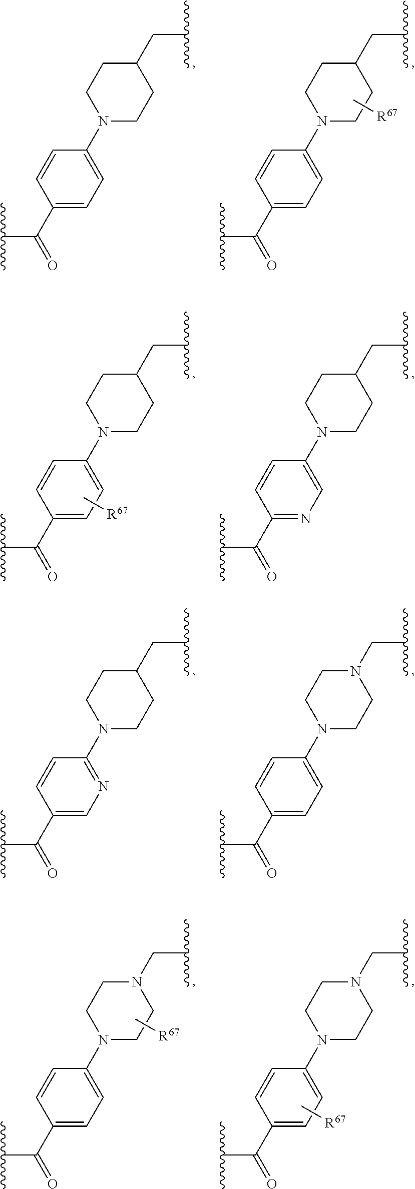

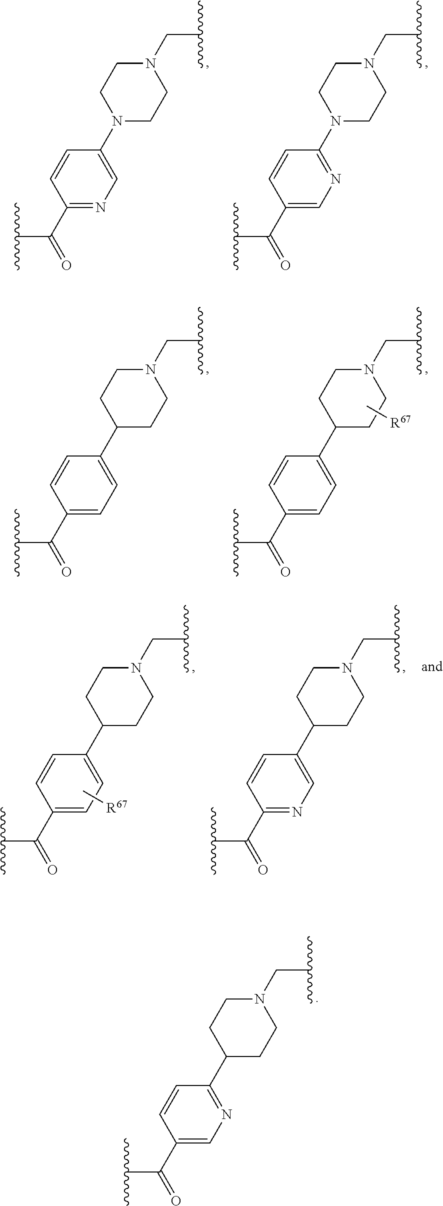

130. The compound of any one of embodiments 1-129, wherein Linker^{B} is:

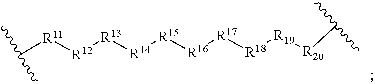

wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, alkenyl, alkynyl, haloalkyl, alkoxy, aryl, heterocycle, and heteroaryl; each of which is optionally substituted with 1, 2, or 3 substituents independently selected from R²¹;
n is independently selected at each instance from 0, 1, 2, 3, 4, 5, 6, 7, 8, 9, or 10;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.
131. The compound of embodiment 130, wherein R¹¹, R¹², R¹³, R¹⁴, R¹⁵, R¹⁶, and R¹⁷ in Linker^{B} are bond.
132. The compound of embodiment 130 or 131, wherein five of R¹¹, R¹², R¹³, R¹⁴, R¹⁵, R¹⁶, R¹⁷, R¹⁸, and R¹⁹ in Linker^{B} are bond.
133. The compound of any one of embodiments 130-132, wherein four of R¹¹, R¹², R¹³, R¹⁴, R¹⁵, R¹⁶, R¹⁷, R¹⁸, and R¹⁹ in Linker^{B} are bond.
134. The compound of any one of embodiments 130-133, wherein R¹⁸, R¹⁹, and R²⁰ are independently selected from bond, alkyl, —C(O)—, —C(O)O—, —OC(O)—, —C(O)NR⁶—, —NR⁶C(O)—, —O—, —S—, —NR⁶—, —C(R²¹R²¹)—, alkenyl, alkynyl, haloalkyl, alkoxy, aryl, heterocycle, and heteroaryl.
135. The compound of any one of embodiments 1-134, wherein the compound is of Formula:

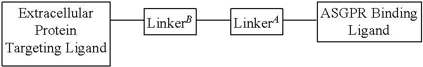

or a pharmaceutically acceptable salt thereof.
136. The compound of embodiment 1, wherein the compound is has the structure of a compound in Table 3.
137. A pharmaceutical composition comprising a compound of any one of embodiments 1 to 136 and a pharmaceutically acceptable carrier.
138. A method of treating a disorder mediated by an Extracellular Protein comprising administering an effective amount of a compound of any one of embodiments 1 to 136 that includes an Extracellular Protein Targeting Ligand that binds to the Extracellular Protein, or a pharmaceutically acceptable salt thereof, to a patient in need thereof.
139. The method of embodiment 138, wherein the extracellular protein is IgA and the disorder is selected from IgA nephropathy (Berger's disease), celiac disease, Crohn's disease, Henoch-Sconiein purpura (HSP), liner IgA bullous dermatosis, IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, a-chain disease, IgA monoclonal gammopathy, monoclonal gammopathy of undetermined significance (MGUS), and linear IgA bullous dermatosis.
140. The method of embodiment 138, wherein the extracellular protein is IgG and the disorder is selected from type 1 autoimmune pancreatitis, interstitial nephritis, Riedel's thyroiditis, storiform fibrosis, Mikulicz's disease, Küttner's tumor, inflammatory pseudotumors, mediastinal fibrosis, retroperitoneal fibrosis (Ormond's disease), aortitis, periaortitis, proximal biliary strictures, idiopathic hypocomplementic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenic gravis, thyroid eye disease, chronic inflammatory demyelinating polyneuropathy, warm autoimmune hemolytic anemia, ankylosing spondylitis, primary Sjögren's syndrome, psoriatic arthritis, and systemic lupus erythematosus (SLE), sclerosing cholangitis, and IgG monoclonal gammopathy, monoclonal gammopathy of undetermined significance (MGUS).
141. The method of embodiment 138, wherein the extracellular protein is IgE and the disorder is selected from atopic asthma, allergic rhinitis, atopic dermatitis, IgE-mediated food allergy, IgE-mediated animal allergies, allergic conjunctivitis, allergic urticaria, anaphylactic shock, nasal polyposis, keratoconjunctivitis, mastocytosis, and eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, and monoclonal gammopathy of undetermined significance (MGUS).
142. The method of embodiment 138, wherein the disorder is dementia or Alzheimer's disease.
143. The method of embodiment 138, wherein the extracellular protein is TNF-α and the disorder is selected from rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.
144. The method of embodiment 138, wherein the extracellular protein is TL-2 and the disorder is selected from host versus graft rejection in transplants and autoimmune disorders.
145. The method of embodiment 138, wherein the extracellular protein is IL-6 and the disorder is selected from Castleman's disease, metastatic castration-associated prostate cancer, renal cell carcinoma, large-cell lung carcinoma, ovarian cancer, rheumatoid arthritis, and asthma.
146. The method of embodiment 138, wherein the extracellular protein is IFN-γ and the disorder is selected from rheumatoid arthritis, multiple sclerosis (MS), corneal transplant rejection, and autoimmune skin diseases.
147. The method of embodiment 138, wherein the disorder is a cancer.
148. A compound selected from:

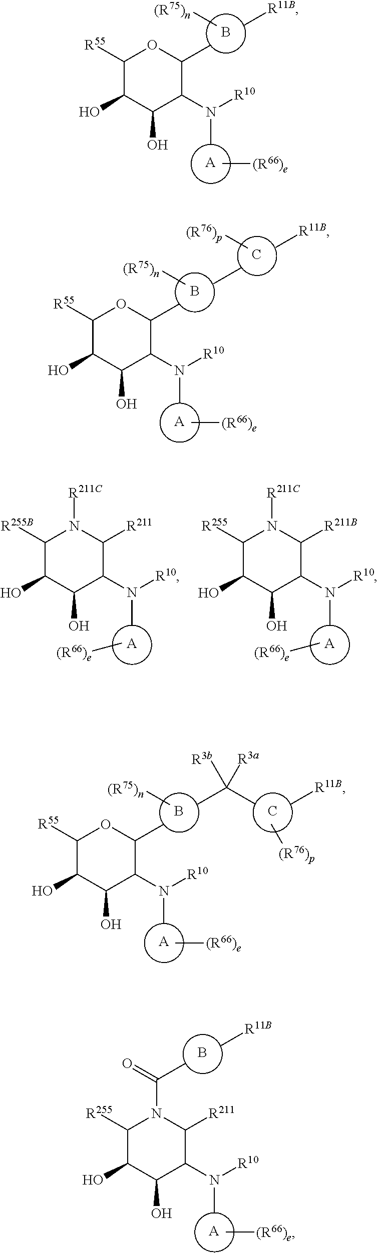

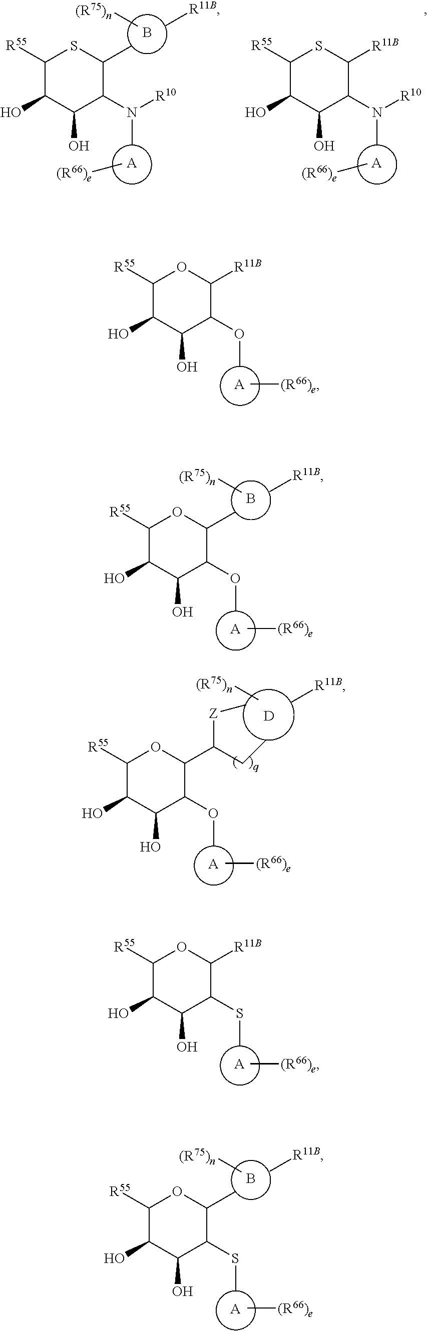

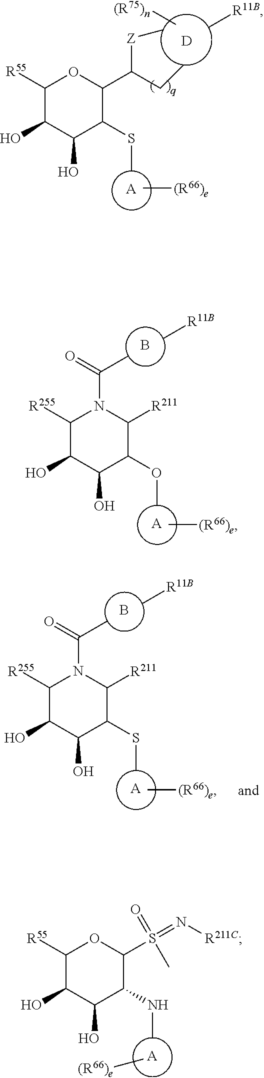

or a salt thereof;
wherein:
R^{11B} and R⁵⁵ are independently selected from hydrogen, C₀-C₆alkyl-cyano, alkyl, alkenyl, alkynyl, haloalkyl, F, Cl, Br, I, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₀-C₆alkylN₃, and R¹¹⁰ each of which is optionally substituted with 1, 2, or 3 substituents independently selected at each occurrence from R⁹⁹;
R³, R^{3a}, R^{3b}, R^{3c}, and R^{3d} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
e is 0, 1, 2, or 3;
q is 1, 2, or 3
\textcircled{A} is heteroaryl;
\textcircled{B} is aryl, heterocycle, cycloalkyl, or heteroaryl;
\textcircled{C} is aryl, heterocycle, cycloalkyl, bicycle, or heteroaryl;
\textcircled{D} is aryl or heteroaryl;
Z is selected from —O—, —NR⁶—, —S—, —S(O)—, —S(O)₂—, and —CR^{3a}R^{3b}—;
R⁶⁶ is independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰;
R⁷⁵ and R⁷⁶ are independently selected at each instance from hydrogen, alkyl, alkenyl, alkynyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, C₀-C₆alkylN₃, heteroaryl, and aryl, each of which is optionally substituted with 1, 2, or 3 substituents independently selected at each occurrence from R¹⁰²;
m and p are independently 0, 1, 2, or 3, as allowed by valence;
R¹¹⁰ is:

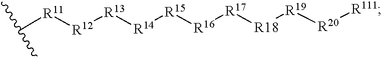

wherein:
R¹¹, R¹², R¹³, R¹⁴, R¹⁵, R¹⁶, R¹, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid; each of which is optionally substituted with 1, 2, or 3 substituents independently selected from R²¹;
n is independently selected at each instance from 0, 1, 2, 3, or 4;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle;
R²⁵⁵, R²¹¹, and R^{211C} are independently selected from hydrogen, C₁-C₆alkyl-cyano, alkyl, alkenyl, alkynyl, haloalkyl, aryl, arylalkyl, heteroaryl, heteroarylalkyl, heterocycle, heterocycloalkyl, haloalkoxy, C₁-C₆alkyl-OR⁶, C₁-C₆alkyl-SR⁶, C₁-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₁-C₆alkylN₃, and R¹¹⁰ each of which is optionally substituted with 1, 2, or 3 substituents independently selected at each occurrence from R⁹⁹; and
R^{255B} and R^{211B} are independently selected from C₁-C₆alkyl-cyano, alkyl, alkenyl, alkynyl, haloalkyl, aryl, arylalkyl, heteroaryl, heteroarylalkyl, heterocycle, heterocycloalkyl, haloalkoxy, C₁-C₆alkyl-OR⁶, C₁-C₆alkyl-SR⁶, C₁-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, C₁-C₆alkylN₃, and R¹¹⁰ each of which is optionally substituted with 1, 2, or 3 substituents independently selected at each occurrence from R⁹⁹.
149. The compound of embodiment 148, wherein one of R^{11B}, R⁵⁵, and R^{66B} is R¹¹⁰.
150. The compound of embodiment 148, wherein none of R^{11B}, R⁵⁵, and R^{66B} are R¹¹⁰.
151. The compound of embodiment 148, wherein one of R²⁵⁵, R²¹¹, and R^{211C} is R¹¹⁰.
152. The compound of embodiment 148, wherein none of R²⁵⁵, R²¹¹, and R^{211C} are R¹¹⁰.
153. The compound of embodiment 148, wherein one of R^{255B} and R^{211B} is R¹¹⁰.
154. The compound of embodiment 148, wherein none of R^{255B} and R^{211B} are R¹¹⁰.
155. The compound of embodiment 148, wherein the compound is selected from:

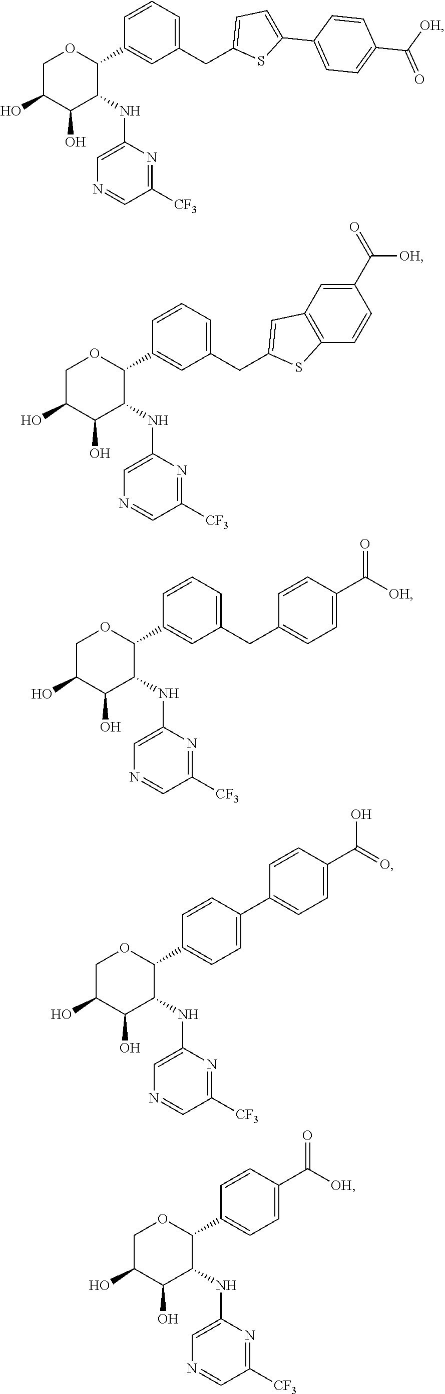 [SMILES: O=C(O)c1ccc(-c2ccc(Cc3cccc([C@H]4OC[C@H](O)[C@H](O)[C@H]4Nc4cncc(C(F)(F)F)n4)c3)s2)cc1 ; O=C(O)c1ccc2sc(Cc3cccc([C@H]4OC[C@H](O)[C@H](O)[C@H]4Nc4cncc(C(F)(F)F)n4)c3)cc2c1 ; O=C(O)c1ccc(Cc2cccc([C@H]3OC[C@H](O)[C@H](O)[C@H]3Nc3cncc(C(F)(F)F)n3)c2)cc1 ; O=C(O)c1ccc(-c2ccc([C@H]3OC[C@H](O)[C@H](O)[C@H]3Nc3cncc(C(F)(F)F)n3)cc2)cc1 ; O=C(O)c1ccc([C@H]2OC[C@H](O)[C@H](O)[C@H]2Nc2cncc(C(F)(F)F)n2)cc1]

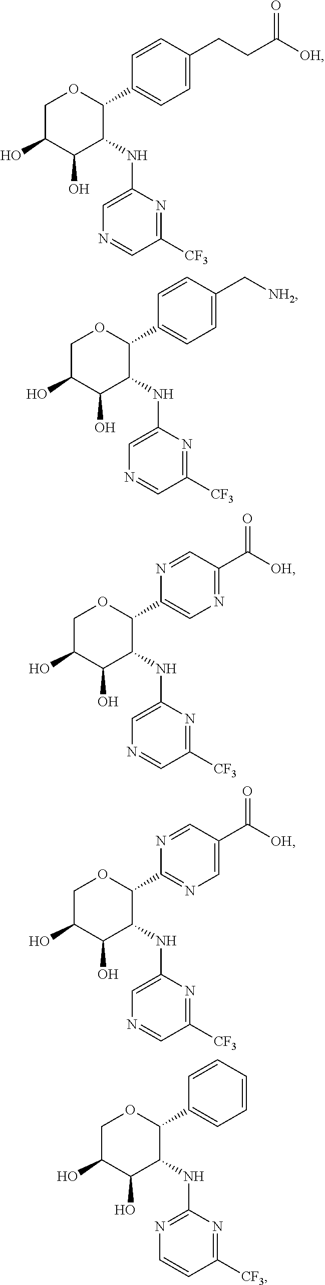

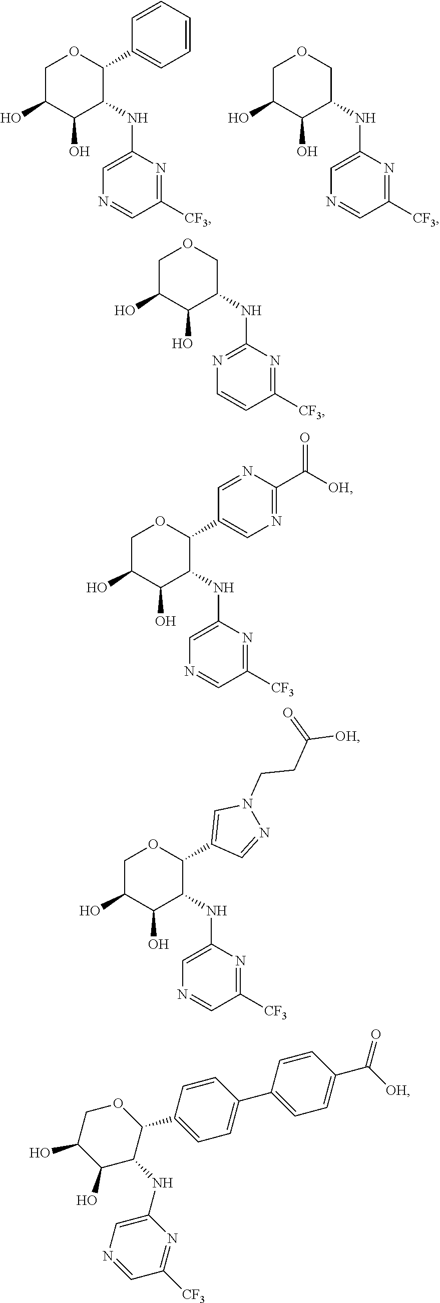

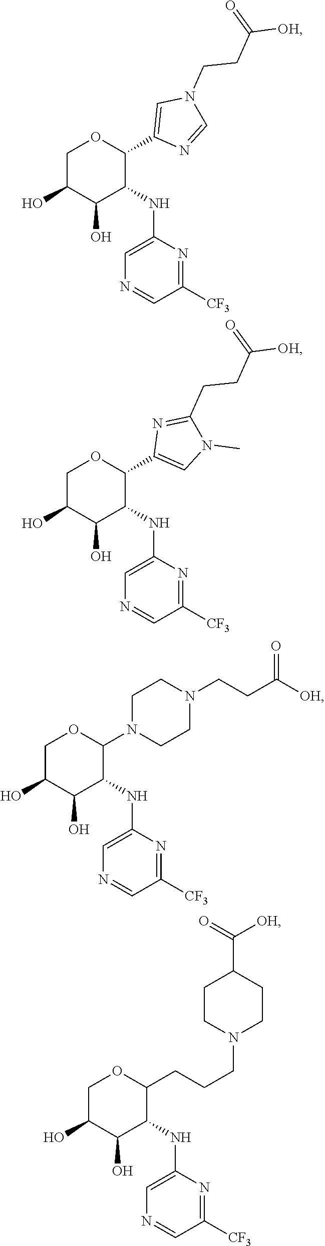

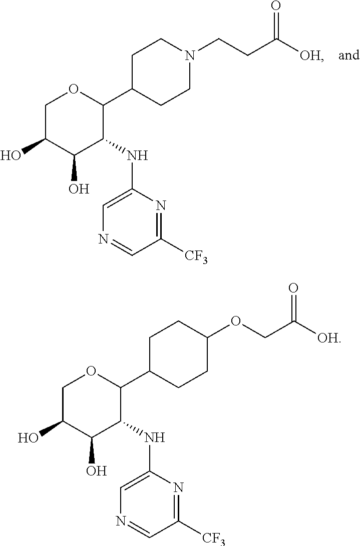 [SMILES: O=C(O)CCN1CCC(C2OC[C@H](O)[C@H](O)[C@H]2Nc2cncc(C(F)(F)F)n2)CC1 ; O=C(O)COC1CCC(C2OC[C@H](O)[C@H](O)[C@H]2Nc2cncc(C(F)(F)F)n2)CC1]

156. The compound of embodiment 148, wherein the compound is selected from:

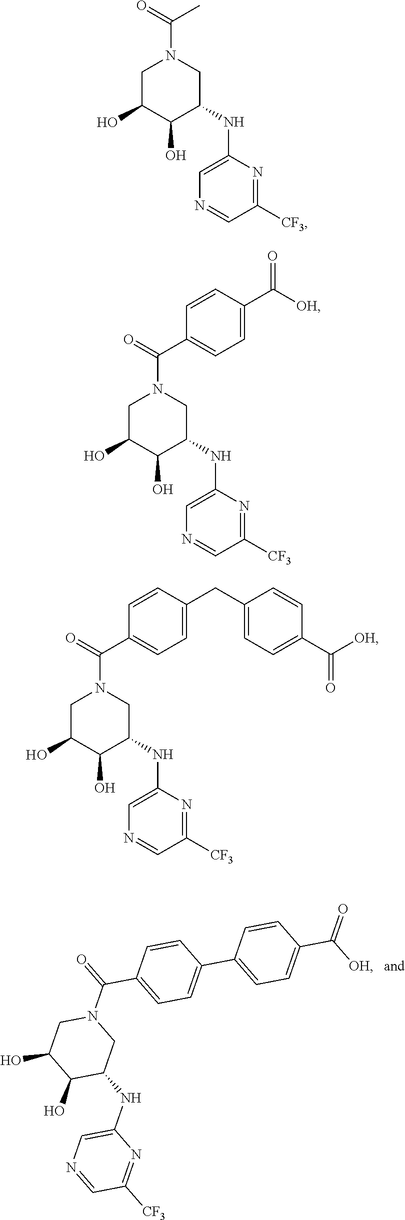

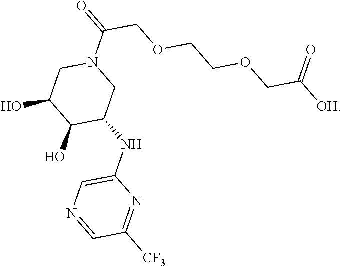 [SMILES: O=C(O)COCCOCC(=O)N1C[C@H](Nc2cncc(C(F)(F)F)n2)[C@@H](O)[C@@H](O)C1]

157. The compound of embodiment 148, wherein the compound is selected from:

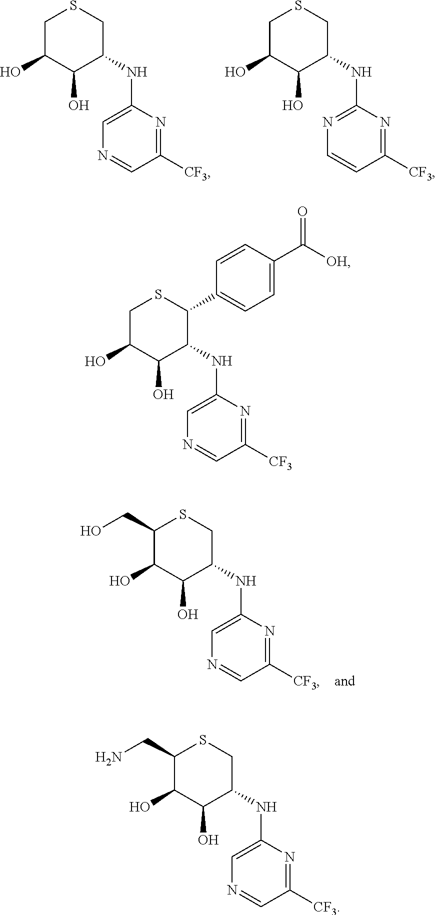 [SMILES: O[C@@H]1[C@@H](Nc2cncc(C(F)(F)F)n2)CSC[C@@H]1O ; O[C@@H]1[C@@H](Nc2nccc(C(F)(F)F)n2)CSC[C@@H]1O ; O=C(O)c1ccc([C@H]2SC[C@H](O)[C@H](O)[C@H]2Nc2cncc(C(F)(F)F)n2)cc1 ; OC[C@H]1SC[C@H](Nc2cncc(C(F)(F)F)n2)[C@@H](O)[C@H]1O ; NC[C@H]1SC[C@H](Nc2cncc(C(F)(F)F)n2)[C@@H](O)[C@H]1O]

158. The compound of embodiment 148, wherein the compound is selected from:

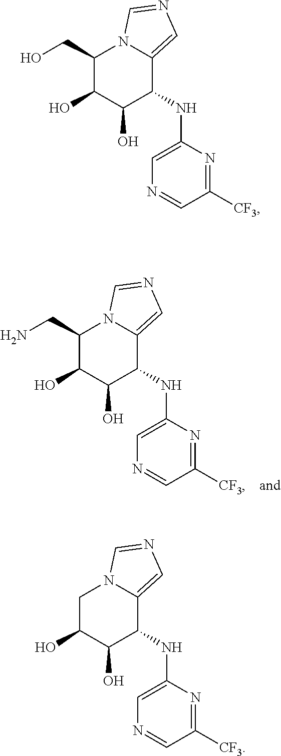 [SMILES: OC[C@@H]1[C@H](O)[C@H](O)[C@@H](Nc2cncc(C(F)(F)F)n2)c2cncn21 ; NC[C@@H]1[C@H](O)[C@H](O)[C@@H](Nc2cncc(C(F)(F)F)n2)c2cncn21 ; O[C@H]1[C@@H](O)Cn2cncc2[C@@H]1Nc1cncc(C(F)(F)F)n1]

159. The compound of embodiment 148, wherein the compound is selected from:

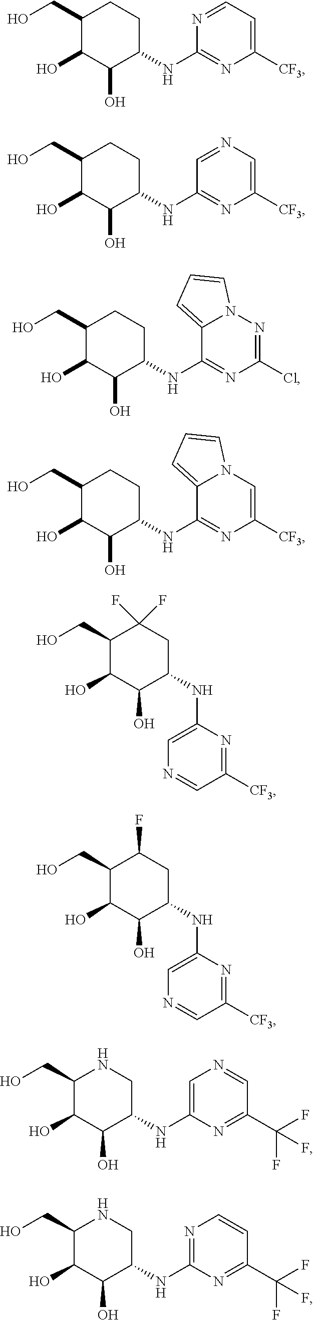

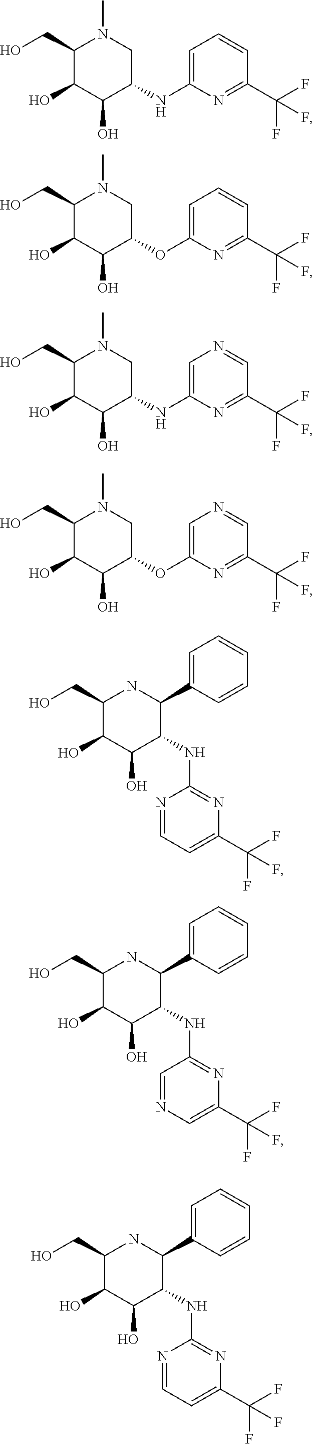

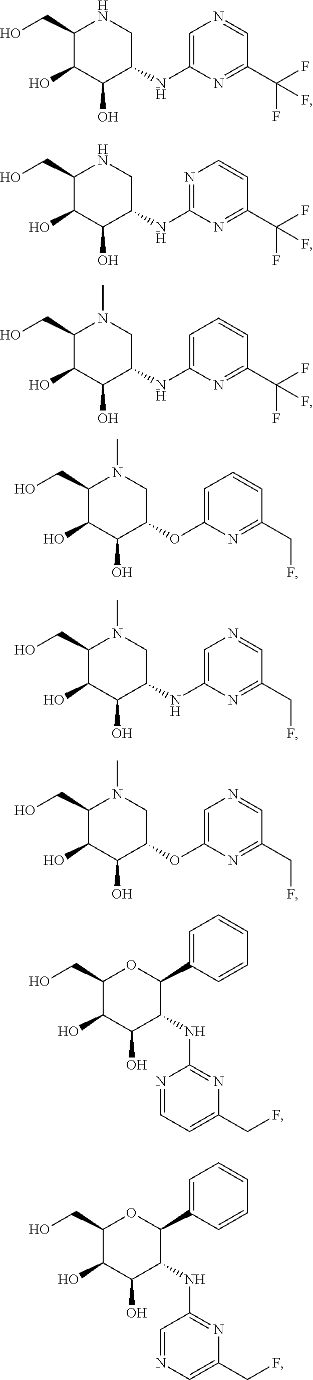

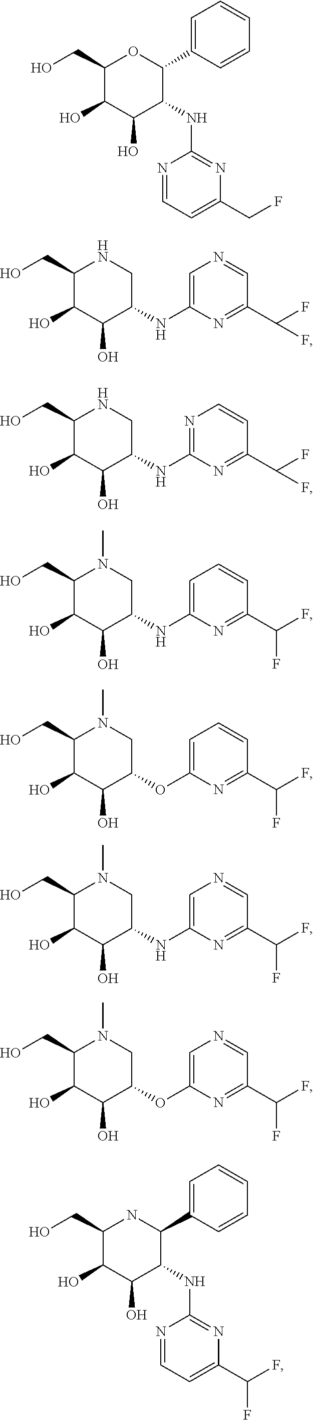

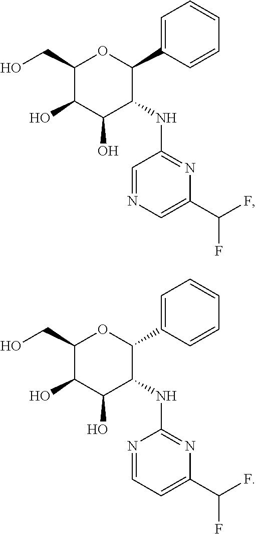

### III. Pharmaceutical Compositions and Dosage Forms for the Extracellular Protein Degraders of the Present Invention

An extracellular protein degrader of the present invention or a pharmaceutically acceptable salt, solvate or prodrug thereof as disclosed herein can be administered as a neat chemical, but is more typically administered as a pharmaceutical composition that includes an effective amount for a host, typically a human, in need of such treatment to treat a disorder mediated by the Target Extracellular Protein as described herein or otherwise well-known for that Target Extracellular Protein.

In certain embodiments, the present invention provides pharmaceutical compositions comprising an extracellular protein degrader of the present invention or a pharmaceutically acceptable composition, salt, isotopic analog such as a deuterated derivative, or prodrug thereof, and a pharmaceutically acceptable excipient. In certain embodiments, the extracellular protein degrader is present in an effective amount, e.g., a therapeutically effective amount or a prophylactically effective amount.

The ASGPR-binding extracellular protein degraders of the present invention can be administered in any manner that allows the degrader to bind to the immunoglobulin, typically in the blood stream, and carry it to the ASGPR-bearing hepatocyte cells on the liver for endocytosis and degradation. As such, examples of methods to deliver the degraders of the present invention include, but are not limited to, oral, intravenous, sublingual, subcutaneous, parenteral, buccal, rectal, intra-aortal, intracranial, subdermal or transnasal, or by other means, in dosage unit formulations containing one or more conventional pharmaceutically acceptable carriers, as appropriate.

In certain embodiments the extracellular protein degrader of the present invention is administered intravenously. Typically, the extracellular protein degrader will be formulated in a liquid dosage form for intravenous injection, such as a buffered solution. Non-limiting examples of solutions for intravenous injection include phosphate buffered solution and saline buffered solution. In certain embodiments the solution is buffered with multiple salts.

In certain embodiments the extracellular protein degrader of the present invention is administered orally. Typically, the extracellular protein degrader will be formulated in a solid dosage form for oral administration or as a gel containing capsule. Non-limiting examples of solid dosage forms include capsules, tablets, and powders.

In certain embodiments the extracellular protein degrader of the present invention is administered subcutaneously. Typically, the extracellular protein degrader will be formulated in a liquid dosage form for subcutaneous injection, such as a buffered solution. Non-limiting examples of solutions for subcutaneous injection include phosphate buffered solution and saline buffered solution. In certain embodiments the solution is buffered with multiple salts.

Therefore, the disclosure provides pharmaceutical compositions comprising an effective amount of extracellular protein degrader or its pharmaceutically acceptable salt together with at least one pharmaceutically acceptable carrier for any appropriate use thereof. The pharmaceutical composition may contain an extracellular protein degrader or salt as the only active agent, or, in an alternative embodiment, the extracellular protein degrader and at least one additional active agent.

In certain embodiments the term pharmaceutically acceptable salt refers to a salt of the described extracellular protein degrader which is, within the scope of sound medical judgment, suitable for administration to a host such as a human without undue toxicity, irritation, allergic response, and the like, commensurate with a reasonable benefit/risk ratio, and effective for its intended use. Thus, the term “pharmaceutically acceptable salt” refers to the relatively non-toxic, inorganic and organic acid addition salts of the presently disclosed extracellular protein degraders. These salts can be prepared during the final isolation and purification of the extracellular protein degraders or by separately reacting the purified extracellular protein degrader in its free form with a suitable organic or inorganic acid and then isolating the salt thus formed. Basic extracellular protein degraders are capable of forming a wide variety of different salts with various inorganic and organic acids. Acid addition salts of the basic extracellular protein degraders are prepared by contacting the free base form with a sufficient amount of the desired acid to produce the salt in the conventional manner. The free base form can be regenerated by contacting the salt form with a base and isolating the free base in the conventional manner. The free base forms may differ from their respective salt forms in certain physical properties such as solubility in polar solvents. Pharmaceutically acceptable base addition salts may be formed with metals or amines, such as alkali and alkaline earth metal hydroxides, or of organic amines. Examples of metals used as cations, include, but are not limited to, sodium, potassium, magnesium, calcium, and the like. Examples of suitable amines include, but are not limited to, N,N′-dibenzylethylenediamine, chloroprocaine, choline, diethanolamine, ethylenediamine, N-methylglucamine, and procaine. The base addition salts of acidic extracellular protein degraders are prepared by contacting the free acid form with a sufficient amount of the desired base to produce the salt in the conventional manner. The free acid form can be regenerated by contacting the salt form with an acid and isolating the free acid in a conventional manner. The free acid forms may differ from their respective salt forms somewhat in certain physical properties such as solubility in polar solvents.

Salts can be prepared from inorganic acids sulfate, pyrosulfate, bisulfate, sulfite, bisulfite, nitrate, phosphate, monohydrogenphosphate, dihydrogenphosphate, metaphosphate, pyrophosphate, chloride, bromide, iodide, nitric, phosphoric, sulfuric, hydrobromic, hydriodic, phosphorus, and the like. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate, laurylsulphonate and isethionate salts, and the like. Salts can also be prepared from organic acids, such as aliphatic mono- and dicarboxylic acids, phenyl-substituted alkanoic acids, hydroxy alkanoic acids, alkanedioic acids, aromatic acids, aliphatic and aromatic sulfonic acids, etc. and the like. Representative salts include acetate, propionate, caprylate, isobutyrate, oxalate, malonate, succinate, suberate, sebacate, fumarate, maleate, mandelate, benzoate, chlorobenzoate, methylbenzoate, dinitrobenzoate, phthalate, benzenesulfonate, toluenesulfonate, phenylacetate, citrate, lactate, maleate, tartrate, methanesulfonate, and the like. Pharmaceutically acceptable salts can include cations based on the alkali and alkaline earth metals, such as sodium, lithium, potassium, calcium, magnesium and the like, as well as non-toxic ammonium, quaternary ammonium, and amine cations including, but not limited to, ammonium, tetramethylammonium, tetraethylammonium, methylamine, dimethylamine, trimethylamine, triethylamine, ethylamine, and the like. Also contemplated are the salts of amino acids such as arginate, gluconate, galacturonate, and the like. See, for example, Berge et al., J. Pharm. Sci., 1977, 66, 1-19, which is incorporated herein by reference.

Pharmaceutically acceptable excipients include inert diluents, dispersing and/or granulating agents, surface active agents and/or emulsifiers, disintegrating agents, binding agents, preservatives, buffering agents, lubricating agents, and/or oils. Additional acceptable excipients include cocoa butter and suppository waxes, coloring agents, coating agents, sweetening, flavoring, perfuming agents, etc., and combinations thereof.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and combinations thereof.

Exemplary granulating and/or dispersing agents include potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, etc., and combinations thereof.

Exemplary surface active agents and/or emulsifiers include natural emulsifiers (e.g. acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g. bentonite [aluminum silicate] and Veegum [magnesium aluminum silicate]), long chain amino acid derivatives, high molecular weight alcohols (e.g. stearyl alcohol, cetyl alcohol, oleyl alcohol, triacetin monostearate, ethylene glycol distearate, glyceryl monostearate, and propylene glycol monostearate, polyvinyl alcohol), carbomers (e.g. carboxy polymethylene, polyacrylic acid, acrylic acid polymer, and carboxyvinyl polymer), carrageenan, cellulosic derivatives (e.g. carboxymethylcellulose sodium, powdered cellulose, hydroxymethyl cellulose, hydroxypropyl cellulose, hydroxypropyl methylcellulose, methylcellulose), sorbitan fatty acid esters (e.g. polyoxyethylene sorbitan monolaurate [Tween 20], polyoxyethylene sorbitan [Tween 60], polyoxyethylene sorbitan monooleate [Tween 80], sorbitan monopalmitate [Span 40], sorbitan monostearate [Span 60], sorbitan tristearate [Span 65], glyceryl monooleate, sorbitan monooleate [Span 80]), polyoxyethylene esters (e.g. polyoxyethylene monostearate [Myrj 45], polyoxyethylene hydrogenated castor oil, polyethoxylated castor oil, polyoxymethylene stearate, and Solutol), sucrose fatty acid esters, polyethylene glycol fatty acid esters (e.g., Cremophor), polyoxyethylene ethers, (e.g. polyoxyethylene lauryl ether [Brij 30]), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic F 68, Poloxamer 188, cetrimonium bromide, cetylpyridinium chloride, benzalkonium chloride, docusate sodium, etc. and/or combinations thereof.

Exemplary binding agents include starch (e.g. cornstarch and starch paste), gelatin, sugars (e.g. sucrose, glucose, dextrose, dextrin, molasses, lactose, lactitol, mannitol, etc.), natural and synthetic gums (e.g. acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum 32 silicate (Veegum), and larch arabogalactan), alginates, polyethylene oxide, polyethylene glycol, inorganic calcium salts, silicic acid, polymethacrylates, waxes, water, alcohol, etc., and/or combinations thereof.

Exemplary preservatives include antioxidants, chelating agents, antimicrobial preservatives, antifungal preservatives, alcohol preservatives, acidic preservatives, etc., and/or combinations thereof.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Any dosage form can be used that achieves the desired results. In certain embodiments the pharmaceutical composition is in a dosage form that contains from about 0.1 mg to about 1500 mg, from about 10 mg to about 1000 mg, from about 100 mg to about 800 mg, or from about 200 mg to about 600 mg of the active extracellular protein degrader and optionally from about 0.1 mg to about 1500 mg, from about 10 mg to about 1000 mg, from about 100 mg to about 800 mg, or from about 200 mg to about 600 mg of an additional active agent in a unit dosage form. Examples are dosage forms with at least about 0.1, 1, 5, 10, 25, 50, 100, 200, 250, 300, 400, 500, 600, 700, 750, 800, 850, 900, 950, or 1,000 mg of active extracellular protein degrader, or its salt. In certain embodiments the dosage form has at most about 0.1, 1, 5, 10, 25, 50, 100, 200, 250, 300, 400, 500, 600, 700, 750, 800, 850, 900, 950, or 1,000 mg of active extracellular protein degrader, or its salt.

In certain embodiments the dose ranges from about 0.01-100 mg/kg of patient bodyweight, for example about 0.01 mg/kg, about 0.05 mg/kg, about 0.1 mg/kg, about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg, about 90 mg/kg, about 95 mg/kg, or about 100 mg/kg.

In some embodiments, extracellular protein degraders disclosed herein or used as described are administered once a day (QD), twice a day (BID), or three times a day (TID). In some embodiments, extracellular protein degraders disclosed herein or used as described are administered at least once a day for at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 35 days, at least 45 days, at least 60 days, at least 75 days, at least 90 days, at least 120 days, at least 150 days, at least 180 days, or longer.

In certain embodiments the extracellular protein degrader of the present invention is administered once a day, twice a day, three times a day, or four times a day.

The pharmaceutical composition may be formulated as any pharmaceutically useful form, e.g., a pill, capsule, tablet, an injection or infusion solution, a syrup, an inhalation formulation, a suppository, a buccal or sublingual formulation, a parenteral formulation, or in a medical device.

Some dosage forms, such as tablets and capsules, can be subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert, or it can possess pharmaceutical benefits of its own. The amount of carrier employed in conjunction with the extracellular protein degrader is sufficient to provide a practical quantity of material for administration per unit dose of the extracellular protein degrader. If provided as in a liquid, it can be a solution or a suspension.

Representative carriers include phosphate buffered saline, water, solvent(s), diluents, pH modifying agents, preservatives, antioxidants, suspending agents, wetting agent, viscosity agents, tonicity agents, stabilizing agents, and combinations thereof. In some embodiments, the carrier is an aqueous carrier. Examples of aqueous carries include, but are not limited to, an aqueous solution or suspension, such as saline, plasma, bone marrow aspirate, buffers, such as Hank's Buffered Salt Solution (HBSS), HEPES (4-(2-hydroxyethyl)-1-piperazineethanesulfonic acid), Ringers buffer, ProVisc®, diluted ProVisc®, Provisc® diluted with PBS, Krebs buffer, Dulbecco's PBS, normal PBS, sodium hyaluronate solution (HA, 5 mg/mL in PBS), citrate buffer, simulated body fluids, plasma platelet concentrate and tissue culture medium or an aqueous solution or suspension comprising an organic solvent. Acceptable solutions include, for example, water, Ringer's solution and isotonic sodium chloride solutions. The formulation may also be a sterile solution, suspension, or emulsion in a non-toxic diluent or solvent such as 1,3-butanediol.

Viscosity agents may be added to the pharmaceutical composition to increase the viscosity of the composition as desired. Examples of useful viscosity agents include, but are not limited to, hyaluronic acid, sodium hyaluronate, carbomers, polyacrylic acid, cellulosic derivatives, polycarbophil, polyvinylpyrrolidone, gelatin, dextrin, polysaccharides, polyacrylamide, polyvinyl alcohol (including partially hydrolyzed polyvinyl acetate), polyvinyl acetate, derivatives thereof and mixtures thereof.

Solutions, suspensions, or emulsions for administration may be buffered with an effective amount necessary to maintain a pH suitable for the selected administration. Suitable buffers are well known by those skilled in the art. Some examples of useful buffers are acetate, borate, carbonate, citrate, and phosphate buffers. Solutions, suspensions, or emulsions for topical, for example, ocular administration may also contain one or more tonicity agents to adjust the isotonic range of the formulation. Suitable tonicity agents are well known in the art. Some examples include glycerin, mannitol, sorbitol, sodium chloride, and other electrolytes.

Classes of carriers include, but are not limited to binders, buffering agents, coloring agents, diluents, disintegrants, emulsifiers, flavorants, glidants, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin, talc, and vegetable oils. Optional active agents may be included in a pharmaceutical composition, which do not substantially interfere with the activity of the extracellular protein degrader of the present invention.

The pharmaceutical compositions/combinations can be formulated for oral administration. These compositions can contain any amount of active extracellular protein degrader that achieves the desired result, for example between 0.1 and 99 weight % (wt. %) of the extracellular protein degrader and usually at least about 5 wt. % of the extracellular protein degrader. Some embodiments contain from about 25 wt. % to about 50 wt. % or from about 5 wt. % to about 75 wt. % of the extracellular protein degrader. Enteric coated oral tablets may also be used to enhance bioavailability of the extracellular protein degraders for an oral route of administration.

Formulations suitable for rectal administration are typically presented as unit dose suppositories. These may be prepared by admixing the active extracellular protein degrader with one or more conventional solid carriers, for example, cocoa butter, and then shaping the resulting mixture.

Extracellular protein degraders of the present invention and pharmaceutically acceptable composition, salts, isotopic analogs, or prodrugs thereof, may be formulated in dosage unit form for ease of administration and uniformity of dosage. It will be understood, however, that the total daily usage of the compositions comprising an extracellular protein degrader as described herein will be decided by the attending physician within the scope of sound medical judgment. The specific therapeutically effective dose level for any particular subject or organism will depend upon a variety of factors including the disease, disorder, or condition being treated and the severity of the disorder; the activity of the specific extracellular protein degrader employed; the specific composition employed; the age, body weight, general health, sex and diet of the subject; the time of administration, route of administration, and rate of excretion of the specific extracellular protein degrader employed; the duration of the treatment; drugs used in combination or coincidental with the specific extracellular protein degrader employed; and like factors well known in the medical arts.

The extracellular protein degraders and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), the condition of the subject (e.g., whether the subject is able to tolerate oral administration).

The exact amount of an extracellular protein degrader required to achieve an effective amount will vary from subject to subject, depending, for example, on species, age, and general condition of a subject, severity of the side effects or disorder, identity of the particular extracellular protein degrader (s), mode of administration, and the like. The desired dosage can be delivered using any frequency determined to be useful by the health care provider, including three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

It will be also appreciated that an extracellular protein degrader or composition, as described herein, can be administered in combination with one or more additional therapeutically active agents. The extracellular protein degraders or compositions can be administered in combination with additional therapeutically active agents that improve their bioavailability, reduce and/or modify their metabolism, inhibit their excretion, and/or modify their distribution within the body. It will also be appreciated that the therapy employed may achieve a desired effect for the same disorder (for example, an extracellular protein degrader can be administered in combination with an anti-inflammatory agent, anti-cancer agent, immunosuppressant, etc.), and/or it may achieve different effects (e.g., control of adverse side-effects, e.g., emesis controlled by an antiemetic).

The extracellular protein degrader or composition can be administered concurrently with, prior to, or subsequent to, one or more additional therapeutically active agents. In general, each agent will be administered at a dose and/or on a time schedule determined for that agent. It will further be appreciated that the additional therapeutically active agent used in this combination can be administered together in a single composition or administered separately in different compositions. The particular combination to employ in a regimen will take into account compatibility of the inventive extracellular protein degrader with the additional therapeutically active agent and/or the desired therapeutic effect to be achieved. In general, it is expected that additional therapeutically active agents used in combination be used at levels that do not exceed the levels at which they are used individually. In some embodiments, the levels used in combination will be lower than those used individually.

Exemplary additional therapeutically active agents include, but are not limited to, small organic molecules such as drug compounds (e.g., compounds approved by the Food and Drugs Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins and cells. In certain embodiments, the additional therapeutically active agent is an anti-cancer agent, e.g., radiation therapy and/or one or more chemotherapeutic agents.

In certain aspects, a treatment regimen is provided comprising the administration of an extracellular protein degrader of the present invention or a pharmaceutically acceptable composition, salt, isotopic analog (such as a deuterated derivative), or prodrug thereof in combination or in alternation with at least one additional therapeutic agent. The combinations and/or alternations can be administered for beneficial, additive, or synergistic effect in the treatment of Target Extracellular Protein-mediated disorders.

### IV. Methods of Treatment

The Targeted Extracellular Proteins of the current invention may include, but are not limited to, immunoglobulins, cytokines, chemokines, growth factors, coagulation factors, extracellular matrix proteins and proteins involved in formation and/or degradation of the extracellular matrix, esterases, lipases, peptidases, convertases, among others. These proteins mediate a range of diseases that can be treated with an effective amount of the disclosed ASGPR-binding Extracellular Protein Degraders described herein.

### Immunoglobulin Mediated Disorders

In certain aspects, a treatment is provided comprising administering an effective amount of an immunoglobulin degrader of the present invention or a pharmaceutically acceptable composition, salt, isotopic analog, or prodrug thereof to a patient with an immunoglobulin-mediated disease.

### Immunoglobulin G IgG

Immunoglobulin G (IgG) is the main type of antibody found in all body fluids (for example, blood and extracellular fluid) and protects against bacterial and viral infections. It represents approximately 75% of serum antibodies in humans and is thus the most common type of antibody found in circulation. IgG antibodies are generated following class switching and maturation of the antibody response, thus they participate predominantly in the secondary immune response. (Vidarsson, Gestur; et al., “IgG subclasses and allotypes: from structure to effector functions”. Frontiers in Immunology, 2014, 5: 520).

IgG can be divided into 4 distinct subclasses (IgG1, IgG2, IgG3, & IgG4). Within each of these two species, the IgG subclasses are 95% identical at the amino acid level. The relatively minor differences have important functional differences. The evolution of IgG subclass switches is regulated by interaction with T cells and follows a 1-way direction (IgG3→IgG1→IgG2→IgG4). (Valenzuela et al., “The Biology of IgG Subclasses and Their Clinical Relevance to Transplantation”, Transplantation, 2018 January; 102(1S Suppl 1):S7-S13) The differences are mainly in the size and configuration of the hinge region, glycosylation sites, and structures, as well as a few key amino acid changes that impact the ability to interact with complement and Fc receptors. These changes, particularly the size of the hinge region, have an impact on the flexibility of the antibody at the hinge.

IgG1 and IgG3 are monomeric (2 heavy chains & 2 light chains) and bivalent (2 variable regions). IgG2 has a distinct disulfide bond pattern which allows for two monomeric IgG2 antibodies to form a dimeric (and tetravalent) structure through unique inter-molecule disulfide bonds. IgG4 has an even more unique structure (again dictated by the heavy chain intrachain disulfide bond). The intrachain disulfide bonds (there are two) can be reduced, which generates a monovalent structure. In addition, the monovalent structures can reform the disulfide bonds, but may not be the same IgG4 monovalent chain; meaning the resulting IgG4 will be a bivalent monomer but will have two different variable regions.

By binding many kinds of pathogens such as viruses, bacteria, and fungi, IgG helps protect the body from infection. However, aberrant IgG activity is associated with a variety of disorders, in these disorders the selective targeting of IgG can be particularly beneficial when the present invention is used in the treatment of a disease known to be caused primarily by IgG, such as thyroid eye disease, myasthenia gravis, chronic inflammatory demyelinating polyneuropathy, warm autoimmune hemolytic anemia, and type-1 autoimmune pancreatitis.

In certain aspects the treatment of a disorder mediated by IgG is provided comprising administering an effective amount of an IgG degrader or a pharmaceutically acceptable salt thereof to the patient. In certain embodiments the IgG disorder is selected from antiphospholipid Ab syndrome, Behcet syndrome, Hashimoto thyroiditis, MGUS, necrobiotic xanthogranuloma, rheumatoid arthritis, cancer, for example multiple myeloma or peripheral multiple myeloma, paraproteinemia, chronic urticaria, scleroderma, scleromyxedema, thrombocytopenia for example heparin-induced thrombocytopenia, cryoglobulinema, granulomatosis with polyanglititis, for example ANCA associated vasculitis, idiopathic thrombocytopenic purpura, thrombocytopenia, IgG4-RD, paroxysmal nocturnal hemoglobinuria (PNH), warm autoimmune hemolytic anemia, rhabdomyolysis, lupus nephritis, acute disseminated encephalomyelitis, Guillaine-Barre syndrome, chronic inflammatory demyelinating polyneuropathy, Miller Fisher syndrome, neuromyelitis optica spectrum disorder, opsoclonus-myoclonus syndrome, pediatric autoimmune neuropsychiatric disorder associated with streptococcal infection (PANDAS), peripheral neuropathy, transverse myelitis, fibrosis, IPF/fibrosis, and transplantation rejection.

In certain embodiments the disease is mediated by IgG.

Immunoglobulin G (IgG) mediates a range of autoimmune, infectious and metabolic diseases, including systemic fibroinflammatory disease. In addition, overexpression of IgG4 is associated with IgG4-related diseases, which generally include multiple organs, and disorders include type 1 autoimmune pancreatitis, interstitial nephritis, Riedel's thyroiditis, storiform fibrosis, Mikulicz's disease, Küttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementemic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenia gravis, ankylosing spondylitis, primary Sjögren's syndrome, psoriatic arthritis, systemic lupus erythematosus (SLE), sclerosing cholangitis, IgG monoclonal gammopathy, monoclonal gammopathy of undetermined significance (MGUS), melanoma, bullous pemphigoid, Goodpasture disease, encephalitis, thrombotic thrombocytopenic purpura, chronic inflammatory polyneuropathy, limbic encephalitis, neuromyotonia, Morvan syndrome, pemphigus foliaceus, pemphigus vulgaris, REM and non-REM parasomnia, and membranous nephropathy, multiple sclerosis, hyperthyroid Grave's disease, epidermolysis bullosa acquisita, pemphigoid gestationis, anti-p200 pemphigoid, and paraneoplastic pemphigus, among others.

In certain embodiments the disease is mediated by IgA Aberrant expression of immunoglobulin A (IgA) mediates a range of autoimmune and immune-mediated disorders, including IgA nephropathy (also known as Berger's disease), celiac disease, Crohn's disease, Henoch-Schonlein purpura (HSP) (also known as IgA vasculitis), IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, a-chain disease, IgA monoclonal gammopathy, monoclonal gammopathy of undetermined significance (MGUS), linear IgA bullous dermatosis, rheumatoid arthritis, ulcerative colitis, and primary glomerulonephritis, among others.

### Immunoglobulin E IgG

In certain embodiments the disease is mediated by IgE.

Immunoglobulin E (IgE) is a strong mediator of allergic disease, including but not limited to, atopic asthma, allergic rhinitis, atopic dermatitis, cutaneous contact hypersensitivity, IgE-mediated food allergy, IgE-mediated animal allergies, allergic conjunctivitis, allergic urticaria, anaphylactic shock, nasal polyposis, keratoconjunctivitis, mastocytosis, eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, monoclonal gammopathy of undetermined significance (MGUS), pemphigus vulgaris, mucus membrane pemphigoid, chronic urticaria, autoimmune uveitis, rheumatoid arthritis, autoimmune pancreatitis, and allergic rhinoconjunctivitis among others.

### Additional Immunoglobulin Disorders

In certain embodiments the disease is mediated by multiple immunoglobulins.

Non-limiting examples of immunoglobulin mediated diseases include: systemic fibroinflammatory disease, type 1 autoimmune pancreatitis, interstitial nephritis, Riedel's thyroiditis, storiform fibrosis, Mikulicz's disease, Küttner's tumor, inflammatory pseudotumors (in various sites of the body), mediastinal fibrosis, retroperitoneal fibrosis (Ormond's disease), aortitis and periaortitis, proximal biliary strictures, idiopathic hypocomplementemic tubulointerstitial nephritis, multifocal fibrosclerosis, pachymeningitis, pancreatic enlargement, tumefactive lesions, pericarditis, rheumatoid arthritis (RA), inflammatory bowel disease, multiple sclerosis, myasthenia gravis, ankylosing spondylitis, primary Sjögren's syndrome, psoriatic arthritis, systemic lupus erythematosus (SLE), sclerosing cholangitis, IgG monoclonal gammopathy, monoclonal gammopathy of undetermined significance (MGUS), melanoma, bullous pemphigoid, Goodpasture disease, encephalitis, thrombotic thrombocytopenic purpura, chronic inflammatory polyneuropathy, limbic encephalitis, neuromyotonia, Morvan syndrome, pemphigus foliaceus, pemphigus vulgaris, REM and non-REM parasomnia, and membranous nephropathy, multiple sclerosis, hyperthyroid Grave's disease, epidermolysis bullosa acquisita, pemphigoid gestationis, anti-p200 pemphigoid, paraneoplastic pemphigus, IgA nephropathy (also known as Berger's disease), celiac disease, Crohn's disease, Henoch-Schonlein purpura (HSP) (also known as IgA vasculitis), IgA pemphigus, dermatitis herpetiformis, inflammatory bowel disease (IBD), Sjögren's syndrome, ankylosing spondylitis, alcoholic liver cirrhosis, acquired immunodeficiency syndrome, IgA multiple myeloma, a-chain disease, IgA monoclonal gammopathy, monoclonal gammopathy of undetermined significance (MGUS), linear IgA bullous dermatosis, rheumatoid arthritis, ulcerative colitis, primary glomerulonephritis, atopic asthma, allergic rhinitis, atopic dermatitis, cutaneous contact hypersensitivity, IgE-mediated food allergy, IgE-mediated animal allergies, allergic conjunctivitis, allergic urticaria, anaphylactic shock, nasal polyposis, keratoconjunctivitis, mastocytosis, eosinophilic gastrointestinal disease, bullous pemphigoid, chemotherapy induced hypersensitivity reaction, seasonal allergic rhinitis, interstitial cystitis, eosinophilic esophagitis, angioedema, acute interstitial nephritis, atopic eczema, eosinophilic bronchitis, chronic obstructive pulmonary disease, gastroenteritis, hyper-IgE syndrome (Job's Syndrome), IgE monoclonal gammopathy, monoclonal gammopathy of undetermined significance (MGUS), pemphigus vulgaris, mucus membrane pemphigoid, chronic urticaria, autoimmune uveitis, rheumatoid arthritis, autoimmune pancreatitis, and allergic rhinoconjunctivitis among others.

### Additional Disorders

Immunoglobulins are also associated with various complex protein signaling cascades, for example the complement cascade, and thus their degradation can treat diseases that are mediated by these protein signaling cascades. Additional examples of disorders that can be treated by compounds of the present invention include autoimmune, other immune dysfunctions, complement mediated disorders, abnormal cellular proliferation, cancer, tumors, hematology-related disorders, renal disorders and liver disorders. In certain embodiments the disorder is mediated by an extracellular protein other than an immunoglobulin and a compound of the present invention that degrades that extracellular protein is administered to a patient in need thereof.

In certain embodiments, the degrader or its salt or composition as described herein is used in the treatment of an autoimmune disorder. In some aspects, the extracellular protein is an Ig, such as IgA or IgG. IgG degradation can treat for example, thyroid eye disease, myasthenia gravis, chronic inflammatory demyelinating polyneuropathy, and warm autoimmune hemolytic anemia.

Non-limiting examples of autoimmune disorders include: lupus, allograft rejection, autoimmune thyroid diseases (such as Graves' disease and Hashimoto's thyroiditis), autoimmune uveoretinitis, giant cell arteritis, inflammatory bowel diseases (including Crohn's disease, ulcerative colitis, regional enteritis, granulomatous enteritis, distal ileitis, regional ileitis, and terminal ileitis), diabetes, multiple sclerosis, pernicious anemia, psoriasis, rheumatoid arthritis, sarcoidosis, and scleroderma.

In an embodiment, the degrader or its salt or composition as described herein is used in the treatment of lupus. Non-limiting examples of lupus include lupus erythematosus, cutaneous lupus, discoid lupus erythematosus, chilblain lupus erythematosus, or lupus erythematosus-lichen planus overlap syndrome. Lupus erythematosus is a general category of disease that includes both systemic and cutaneous disorders. The systemic form of the disease can have cutaneous as well as systemic manifestations. However, there are also forms of the disease that are only cutaneous without systemic involvement. For example, SLE is an inflammatory disorder of unknown etiology that occurs predominantly in women, and is characterized by articular symptoms, butterfly erythema, recurrent pleurisy, pericarditis, generalized adenopathy, splenomegaly, as well as CNS involvement and progressive renal failure. The sera of most patients (over 98%) contain antinuclear antibodies, including anti-DNA antibodies. High titers of anti-DNA antibodies are essentially specific for SLE. Conventional treatment for this disease has been the administration of corticosteroids or immunosuppressants.

There are three forms of cutaneous lupus: chronic cutaneous lupus (also known as discoid lupus erythematosus or DLE), subacute cutaneous lupus, and acute cutaneous lupus. DLE is a disfiguring chronic disorder primarily affecting the skin with sharply circumscribed macules and plaques that display erythema, follicular plugging, scales, telangiectasia and atrophy. The condition is often precipitated by sun exposure, and the early lesions are erythematous, round scaling papules that are 5 to 10 mm in diameter and display follicular plugging. DLE lesions appear most commonly on the cheeks, nose, scalp, and ears, but they may also be generalized over the upper portion of the trunk, extensor surfaces of the extremities, and on the mucous membranes of the mouth. If left untreated, the central lesion atrophies and leaves a scar. Unlike SLE, antibodies against double-stranded DNA (e.g., DNA-binding test) are almost invariably absent in DLE.

Multiple Sclerosis is an autoimmune demyelinating disorder that is believed to be T lymphocyte dependent. MS generally exhibits a relapsing-remitting course or a chronic progressive course. The etiology of MS is unknown, however, viral infections, genetic predisposition, environment, and autoimmunity all appear to contribute to the disorder. Lesions in MS patients contain infiltrates of predominantly T lymphocyte mediated microglial cells and infiltrating macrophages. CD4+ T lymphocytes are the predominant cell type present at these lesions. The hallmark of the MS lesion is plaque, an area of demyelination sharply demarcated from the usual white matter seen in MRI scans. Histological appearance of MS plaques varies with different stages of the disease. In active lesions, the blood-brain barrier is damaged, thereby permitting extravasation of serum proteins into extracellular spaces. Inflammatory cells can be seen in perivascular cuffs and throughout white matter. CD4+ T-cells, especially Th1, accumulate around postcapillary venules at the edge of the plaque and are also scattered in the white matter. In active lesions, up-regulation of adhesion molecules and markers of lymphocyte and monocyte activation, such as IL2-R and CD26 have also been observed. Demyelination in active lesions is not accompanied by destruction of oligodendrocytes. In contrast, during chronic phases of the disease, lesions are characterized by a loss of oligodendrocytes and hence, the presence of myelin oligodendrocyte glycoprotein (MOG) antibodies in the blood.

Diabetes can refer to either type 1 or type 2 diabetes. In some embodiments the degrader or its salt or composition as described herein is provided at an effective dose to treat a patient with type 1 diabetes. In certain aspects the degrader or its salt or composition as described herein is provided at an effective dose to treat a patient with type 2 diabetes.

Type 1 diabetes is an autoimmune disease. An autoimmune disease results when the body's system for fighting infection (the immune system) turns against a part of the body. Type 1 diabetes often occurs when the pancreas is damaged by the immune system. The damaged pancreas then produces little or no insulin.

As examples, the degrader or its salt or composition as described herein is useful for treating or preventing a disorder selected from autoimmune oophoritis, endometriosis, autoimmune orchitis, Ord's thyroiditis, autoimmune enteropathy, coeliac disease, Hashimoto's encephalopathy, antiphospholipid syndrome (APLS) (Hughes syndrome), aplastic anemia, autoimmune lymphoproliferative syndrome (Canale-Smith syndrome), autoimmune neutropenia, Evans syndrome, pernicious anemia, pure red cell aplasia, thrombocytopenia, adipose dolorosa (Dercum's disease), adult onset Still's disease, ankylosing spondylitis, CREST syndrome, drug-induced lupus, eosinophilic fasciitis (Shulman's syndrome), Felty syndrome, IgG4-related disease, mixed connective tissue disease (MCTD), palindromic rheumatism (Hench-Rosenberg syndrome), Parry-Romberg syndrome, Parsonage-Turner syndrome, relapsing polychondritis (Meyenburg-Altherr-Uehlinger syndrome), retroperitonial fibrosis, rheumatic fever, Schnitzler syndrome, fibromyalgia, neuromyotonia (Isaac's disease), paraneoplastic degeneration, autoimmune inner ear disease, Meniere's disease, interstitial cystitis, autoimmune pancreatitis, zika virus-related disorders, chikungunya virus-related disorders, subacute bacterial endocarditis (SBE), IgA nephropathy, IgA vasculitis, polymyalgia rheumatic, rheumatoid vasculitis, alopecia areata, autoimmune progesterone dermatitis, dermatitis herpetiformis, erythema nodosum, gestational pemphigoid, hidradenitis suppurativa, lichen sclerosus, linear IgA disease (LAD), morphea, myositis, pityriasis lichenoides et varioliformis acuta, vitiligo post-myocardial infarction syndrome (Dressler's syndrome), post-pericardiotomy syndrome, autoimmune retinopathy, Cogan syndrome, Graves opthalmopathy, ligneous conjunctivitis, Mooren's ulcer, opsoclonus myoclonus syndrome, optic neuritis, retinocochleocerebral vasculopathy (Susac's syndrome), sympathetic opthalmia, Tolosa-Hunt syndrome, interstitial lung disease, antisynthetase syndrome, Addison's disease, autoimmune polyendocrine syndrome (APS) type I, autoimmune polyendocrine syndrome (APS) type II, autoimmune polyendocrine syndrome (APS) type III, disseminated sclerosis (multiple sclerosis, pattern II), rapidly progressing glomerulonephritis (RPGN), juvenile rheumatoid arthritis, enthesitis-related arthritis, reactive arthritis (Reiter's syndrome), autoimmune hepatitis or lupoid hepatitis, primary biliary cirrhosis (PBS), primary sclerosing cholangitis, microscopic colitis, latent lupus (undifferentiated connective tissue disease (UCTD)), acute disseminated encephalomyelitis (ADEM), acute motor axonal neuropathy, anti-n-methyl-D-aspartate receptor encephalitis, Balo concentric sclerosis (Schilders disease), Bickerstaff's encephalitis, chronic inflammatory demyelinating polyneuropathy, idiopathic inflammatory demyelinating disease, Lambert-Eaton mysathenic syndrome, Oshtoran syndrome, pediatric autoimmune neuropsychiatric disorder associated with streptococcus (PANDAS), progressive inflammatory neuropathy, restless leg syndrome, stiff person syndrome, Sydenhem syndrome, transverse myelitis, lupus vasculitis, leukocytoclastic vasculitis, Microscopic Polyangiitis, polymyositis or ischemic-reperfusion injury of the eye.

In some aspects, the disorder treated by the degrader or its salt or composition as described herein is selected from fatty liver and conditions stemming from fatty liver, such as nonalcoholic steatohepatitis (NASH), liver inflammation, cirrhosis and liver failure.

In other embodiments, the degrader or its salt or composition as described herein is used to modulate an immune response prior to or during surgery or other medical procedure. Non-limiting examples are the use in connection with acute or chronic graft versus host disease, which is a common complication as a result of allogeneic tissue transplant, and can also occur as a result of a blood transfusion.

In certain embodiments, the present invention provides a method of treating or preventing dermatomyositis by administering to a subject in need thereof an effective amount of the degrader or its salt or composition as described herein.

In certain embodiments, the present invention provides a method of treating or preventing amyotrophic lateral sclerosis by administering to a subject in need thereof an effective amount of the degrader or its salt or composition as described herein.

In certain embodiments, the present invention provides a method of treating or preventing abdominal aortic aneurysm, hemodialysis complications, hemolytic anemia, or hemodialysis by administering to a subject in need thereof an effective amount of the degrader or its salt or composition as described herein.

In certain embodiments, a method is provided for the treatment or prevention of cytokine or inflammatory reactions in response to the administration of pharmaceutical or biotherapeutic (e.g. CAR T-cell therapy or monoclonal antibody therapy) in a host by administering an effective amount of the degrader or its salt or composition as described herein. Various types of cytokine or inflammatory reactions may occur in response to a number of factors, such as the administrations of biotherapeutics. In certain aspects, the cytokine or inflammatory reaction is cytokine release syndrome. In certain embodiments, the cytokine or inflammatory reaction is tumor lysis syndrome (which also leads to cytokine release). Symptoms of cytokine release syndrome range from fever, headache, and skin rashes to bronchospasm, hypotension and even cardiac arrest. Severe cytokine release syndrome is described as cytokine storm, and can be fatal.

In another embodiment, the disorder is episcleritis, idiopathic episcleritis, anterior episcleritis, or posterior episcleritis. In certain embodiments, the disorder is idiopathic anterior uveitis, HLA-B27 related uveitis, herpetic keratouveitis, Posner Schlossman syndrome, Fuch's heterochromic iridocyclitis, or cytomegalovirus anterior uveitis.

In another embodiment, the present invention provides a method of treating or preventing a C3 glomurenopathy by administering to a subject in need thereof an effective amount of the degrader or its salt or composition as described herein. In certain embodiments, the disorder is selected from dense deposit disease (DDD) and C3 glomerulonephritis (C3GN).

In yet another embodiment, the present invention provides a method of treating or preventing a IC-MPGN by administering to a subject in need thereof an effective amount of the degrader or its salt or composition as described herein.

In a further embodiment, the present invention provides a method of treating or preventing a paroxysmal nocturnal hemoglobinuria (PNH) by administering to a subject in need thereof an effective amount of the degrader or its salt or composition as described herein.

In another embodiment, the present invention provides a method of treating or preventing age-related macular degeneration (AMD) by administering to a subject in need thereof an effective amount of the degrader or its salt or composition as described herein.

In certain embodiments, the present invention provides a method of treating or preventing rheumatoid arthritis by administering to a subject in need thereof an effective amount of the degrader or its salt or composition as described herein.

In certain embodiments, the present invention provides a method of treating or preventing multiple sclerosis by administering to a subject in need thereof an effective amount of the degrader or its salt or composition as described herein.

In certain embodiments, the present invention provides a method of treating or preventing myasthenia gravis by administering to a subject in need thereof an effective amount of the degrader or its salt or composition as described herein.

In certain embodiments, the present invention provides a method of treating or preventing atypical hemolytic uremic syndrome (aHUS) by administering to a subject in need thereof an effective amount of the degrader or its salt or composition as described herein.

In certain embodiments, the present invention provides a method of treating or preventing neuromyelitis optica (NMO) by administering to a subject in need thereof an effective amount of the degrader or its salt or composition as described herein.

In yet other embodiments, the present invention provides a method of treating or preventing a disorder as described below by administering to a subject in need thereof an effective amount of the degrader or its salt or composition as described herein, including: for example: vitritis, sarcoidosis, syphilis, tuberculosis, or Lyme disease; retinal vasculitis, Eales disease, tuberculosis, syphilis, or toxoplasmosis; neuroretinitis, viral retinitis, or acute retinal necrosis; varicella zoster virus, herpes simplex virus, cytomegalovirus, Epstein-Barr virus, lichen planus, or Dengue-associated disease (e.g., hemorraghic Dengue Fever); Masquerade syndrome, contact dermatitis, trauma induced inflammation, UVB induced inflammation, eczema, granuloma annulare, or acne.

In additional embodiments, the disorder is selected from: acute myocardial infarction, aneurysm, cardiopulmonary bypass, dilated cardiomyopathy, complement activation during cardiopulmonary bypass operations, coronary artery disease, restenosis following stent placement, or percutaneous transluminal coronary angioplasty (PTCA); antibody-mediated transplant rejection, anaphylactic shock, anaphylaxis, allogenic transplant, humoral and vascular transplant rejection, graft dysfunction, graft-versus-host disease, Graves' disease, adverse drug reactions, or chronic graft vasculopathy; allergic bronchopulmonary aspergillosis, allergic neuritis, drug allergy, radiation-induced lung injury, eosinophilic pneumonia, radiographic contrast media allergy, bronchiolitis obliterans, or interstitial pneumonia; parkinsonism-dementia complex, sporadic frontotemporal dementia, frontotemporal dementia with Parkinsonism linked to chromosome 17, frontotemporal lobar degeneration, tangle only dementia, cerebral amyloid angiopathy, cerebrovascular disorder, certain forms of frontotemporal dementia, chronic traumatic encephalopathy (CTE), PD with dementia (PDD), argyrophilic grain dementia, dementia pugilistica, dementia with Lewy Bodies (DLB), or multi-infarct dementia; Creutzfeldt-Jakob disease, Huntington's disease, multifocal motor neuropathy (MMN), prion protein cerebral amyloid angiopathy, polymyositis, postencephalitic parkinsonism, subacute sclerosing panencephalitis, non-Guamanian motor neuron disease with neurofibrillary tangles, neural regeneration, or diffuse neurofibrillary tangles with calcification.

In further embodiments, the disorder is selected from: atopic dermatitis, dermatitis, dermatomyositis bullous pemphigoid, scleroderma, sclerodermatomyositis, psoriatic arthritis, pemphigus vulgaris, Discoid lupus erythematosus, cutaneous lupus, chilblain lupus erythematosus, or lupus erythematosus-lichen planus overlap syndrome; cryoglobulinemic vasculitis, mesenteric/enteric vascular disorder, peripheral vascular disorder, antineutrophil cytoplasm antibody (ANCA)-associated vasculitis (AAV), TL-2 induced vascular leakage syndrome, or immune complex vasculitis; angioedema, low platelets (HELLP) syndrome, sickle cell disease, platelet refractoriness, red cell casts, or typical or infectious hemolytic uremic syndrome (tHUS); hematuria, hemorrhagic shock, drug-induced thrombocytopenia, autoimmune hemolytic anemia (AIHA), azotemia, blood vessel and/or lymph vessel inflammation, rotational atherectomy, or delayed hemolytic transfusion reaction; British type amyloid angiopathy, Buerger's disease, bullous pemphigoid, C1q nephropathy, cancer, or catastrophic antiphospholipid syndrome.

In other embodiments, the disorder is selected from: wet (exudative) AMD, dry (non-exudative) AMD, chorioretinal degeneration, choroidal neovascularization (CNV), choroiditis, loss of RPE function, loss of vision (including loss of visual acuity or visual field), loss of vision from AMD, retinal damage in response to light exposure, retinal degeneration, retinal detachment, retinal dysfunction, retinal neovascularization (RNV), retinopathy of prematurity, pathological myopia, or RPE degeneration; pseudophakic bullous keratopathy, symptomatic macular degeneration related disorder, optic nerve degeneration, photoreceptor degeneration, cone degeneration, loss of photoreceptor cells, pars planitis, scleritis, proliferative vitreoretinopathy, or formation of ocular drusen; chronic urticaria, Churg-Strauss syndrome, cold agglutinin disease (CAD), corticobasal degeneration (CBD), cryoglobulinemia, cyclitis, damage of the Bruch's membrane, Degos disease, diabetic angiopathy, elevated liver enzymes, endotoxemia, epidermolysis bullosa, or epidermolysis bullosa acquisita; essential mixed cryoglobulinemia, excessive blood urea nitrogen-BUN, focal segmental glomerulosclerosis, Gerstmann-Straussler-Scheinker disease, giant cell arteritis, gout, Hallervorden-Spatz disease, Hashimoto's thyroiditis, Henoch-Schonlein purpura nephritis, or abnormal urinary sediments; hepatitis, hepatitis A, hepatitis B, hepatitis C or human immunodeficiency virus (HIV), a viral infection more generally, for example selected from Flaviviridae, Retroviruses, Coronaviridae, Poxviridae, Adenoviridae, Herpesviridae, Caliciviridae, Reoviridae, Picornaviridae, Togaviridae, Orthomyxoviridae, Rhabdoviridae, or Hepadnaviridae; Neisseria meningitidis, shiga toxin E. coli-related hemolytic uremic syndrome (STEC-HUS), hemolytic uremic syndrome (HUS); Streptococcus, or poststreptococcal glomerulonephritis.

In further embodiments, the disorder is selected from: hyperlipidemia, hypertension, hypoalbuminemia, hypobolemic shock, hypocomplementemic urticarial vasculitis syndrome, hypophosphastasis, hypovolemic shock, idiopathic pneumonia syndrome, or idiopathic pulmonary fibrosis; inclusion body myositis, intestinal ischemia, iridocyclitis, iritis, juvenile chronic arthritis, Kawasaki's disease (arteritis), or lipiduria; membranoproliferative glomerulonephritis (MPGN) I, microscopic polyangiitis, mixed cryoglobulinemia, molybdenum cofactor deficiency (MoCD) type A, pancreatitis, panniculitis, Pick's disease, polyarteritis nodosa (PAN), progressive subcortical gliosis, proteinuria, reduced glomerular filtration rate (GFR), or renovascular disorder; multiple organ failure, multiple system atrophy (MSA), myotonic dystrophy, Niemann-Pick disease type C, chronic demyelinating diseases, or progressive supranuclear palsy; spinal cord injury, spinal muscular atrophy, spondyloarthropathies, Reiter's syndrome, spontaneous fetal loss, recurrent fetal loss, pre-eclampsia, synucleinopathy, Takayasu's arteritis, post-partum thryoiditis, thyroiditis, Type I cryoglobulinemia, Type II mixed cryoglobulinemia, Type III mixed cryoglobulinemia, ulcerative colitis, uremia, urticaria, venous gas embolus (VGE), or Wegener's granulomatosis; von Hippel-Lindau disease, histoplasmosis of the eye, hard drusen, soft drusen, pigment clumping, or photoreceptor and/or retinal pigmented epithelia (RPE) loss.

Examples of eye disorders that may be treated according to the compositions and methods disclosed herein include amoebic keratitis, fungal keratitis, bacterial keratitis, viral keratitis, onchorcercal keratitis, bacterial keratoconjunctivitis, viral keratoconjunctivitis, corneal dystrophic diseases, Fuchs' endothelial dystrophy, Sjögren's syndrome, Stevens-Johnson syndrome, autoimmune dry eye diseases, environmental dry eye diseases, corneal neovascularization diseases, post-corneal transplant rejection prophylaxis and treatment, autoimmune uveitis, infectious uveitis, posterior uveitis (including toxoplasmosis), pan-uveitis, an inflammatory disease of the vitreous or retina, endophthalmitis prophylaxis and treatment, macular edema, macular degeneration, age related macular degeneration, proliferative and non-proliferative diabetic retinopathy, hypertensive retinopathy, an autoimmune disease of the retina, primary and metastatic intraocular melanoma, other intraocular metastatic tumors, open angle glaucoma, closed angle glaucoma, pigmentary glaucoma and combinations thereof.

In other embodiments, the disorder is selected from glaucoma, diabetic retinopathy, blistering cutaneous diseases (including bullous pemphigoid, pemphigus, and epidermolysis bullosa), ocular cicatrical pemphigoid, uveitis, adult macular degeneration, diabetic retinopa retinitis pigmentosa, macular edema, diabetic macular edema, Behcet's uveitis, multifocal choroiditis, Vogt-Koyangi-Harada syndrome, intermediate uveitis, birdshot retino-chorioditis, sympathetic ophthalmia, ocular dicatricial pemphigoid, ocular pemphigus, nonartertic ischemic optic neuropathy, postoperative inflammation, and retinal vein occlusion, or central retinal vein occlusion (CVRO).

Disorders that may be treated or prevented by the degrader or its salt or composition as described herein also include, but are not limited to: hereditary angioedema, capillary leak syndrome, hemolytic uremic syndrome (HUS), neurological disorders, Guillain Barre Syndrome, diseases of the central nervous system and other neurodegenerative conditions, glomerulonephritis (including membrane proliferative glomerulonephritis), SLE nephritis, proliferative nephritis, liver fibrosis, tissue regeneration and neural regeneration, or Barraquer-Simons Syndrome; inflammatory effects of sepsis, systemic inflammatory response syndrome (SIRS), disorders of inappropriate or undesirable complement activation, interleukin-2 induced toxicity during IL-2 therapy, inflammatory disorders, inflammation of autoimmune diseases, system lupus erythematosus (SLE), lupus nephritides, arthritis, immune complex disorders and autoimmune diseases, systemic lupus, or lupus erythematosus; ischemia/reperfusion injury (I/R injury), myocardial infarction, myocarditis, post-ischemic reperfusion conditions, balloon angioplasty, atherosclerosis, post-pump syndrome in cardiopulmonary bypass or renal bypass, renal ischemia, mesenteric artery reperfusion after aortic reconstruction, antiphospholipid syndrome, autoimmune heart disease, ischemia-reperfusion injuries, obesity, or diabetes; Alzheimer's dementia, stroke, schizophrenia, traumatic brain injury, trauma, Parkinson's disease, epilepsy, transplant rejection, prevention of fetal loss, biomaterial reactions (e.g. in hemodialysis, implants), hyperacute allograft rejection, xenograft rejection, transplantation, psoriasis, burn injury, thermal injury including burns or frostbite, or crush injury; asthma, allergy, acute respiratory distress syndrome (ARDS), cystic fibrosis, adult respiratory distress syndrome, dyspnea, hemoptysis, chronic obstructive pulmonary disease (COPD), emphysema, pulmonary embolisms and infarcts, pneumonia, fibrogenic dust diseases, inert dusts and minerals (e.g., silicon, coal dust, beryllium, and asbestos), pulmonary fibrosis, organic dust diseases, chemical injury (due to irritant gases and chemicals, e.g., chlorine, phosgene, sulfur dioxide, hydrogen sulfide, nitrogen dioxide, ammonia, and hydrochloric acid), smoke injury, thermal injury (e.g., burn, freeze), bronchoconstriction, hypersensitivity pneumonitis, parasitic diseases, Goodpasture's Syndrome (anti-glomerular basement membrane nephritis), pulmonary vasculitis, Pauci-immune vasculitis, or immune complex-associated inflammation.

In another embodiment, a method for the treatment of sickle cell in a host is provided that includes the administration of an effective amount of the degrader or its salt or composition as described herein. In certain embodiments, a method for the treatment of immunothrombocytopenic purpura (ITP), thrombotic thrombocytopenic purpura (TTP), or idiopathic thrombocytopenic purpura (ITP) in a host is provided that includes the administration of an effective amount of the degrader or its salt or composition as described herein. In certain embodiments, a method for the treatment of ANCA-vasculitis in a host is provided that includes the administration of an effective amount of the degrader or its salt or composition as described herein. In certain embodiments, a method for the treatment of IgA nephropathy in a host is provided that includes the administration of an effective amount of the degrader or its salt or composition as described herein. In certain embodiments, a method for the treatment of rapidly progressing glomerulonephritis (RPGN), in a host is provided that includes the administration of an effective amount of the degrader or its salt or composition as described herein. In certain embodiments, a method for the treatment of lupus nephritis, in a host is provided that includes the administration of an effective amount of the degrader or its salt or composition as described herein. In certain embodiments, a method for the treatment of hemorraghic dengue fever, in a host is provided that includes the administration of an effective amount of the degrader or its salt or composition as described herein.

In other aspects, an effective amount of the degrader or its salt or composition as described herein is used to treat an abnormal proliferation disorder such as a tumor or cancer.

Non-limiting examples of cancers that can be treated according to the present invention include, but are not limited to, acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarcinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL)—also known as acute lymphoblastic leukemia or acute lymphoid leukemia (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt's lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CVL), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva).

In another embodiment, the disorder is myelodysplastic syndrome (MDS).

In certain embodiments, the cancer is a hematopoietic cancer. In certain embodiments, the hematopoietic cancer is a lymphoma. In certain embodiments, the hematopoietic cancer is a leukemia. In certain embodiments, the leukemia is acute myelocytic leukemia (AML).

In certain embodiments, the proliferative disorder is a myeloproliferative neoplasm. In certain embodiments, the myeloproliferative neoplasm (MPN) is primary myelofibrosis (PMF).

In certain embodiments, the cancer is a solid tumor. A solid tumor, as used herein, refers to an abnormal mass of tissue that usually does not contain cysts or liquid areas. Different types of solid tumors are named for the type of cells that form them. Examples of classes of solid tumors include, but are not limited to, sarcomas, carcinomas, and lymphomas, as described above herein. Additional examples of solid tumors include, but are not limited to, squamous cell carcinoma, colon cancer, breast cancer, prostate cancer, lung cancer, liver cancer, pancreatic cancer, and melanoma.

Abnormal cellular proliferation, notably hyperproliferation, can occur as a result of a wide variety of factors, including genetic mutation, infection, exposure to toxins, autoimmune disorders, and benign or malignant tumor induction.

There are a number of skin disorders associated with cellular hyperproliferation. Psoriasis, for example, is a benign disease of human skin generally characterized by plaques covered by thickened scales. The disease is caused by increased proliferation of epidermal cells of unknown cause. Chronic eczema is also associated with significant hyperproliferation of the epidermis.

Other diseases caused by hyperproliferation of skin cells include atopic dermatitis, lichen planus, warts, pemphigus vulgaris, actinic keratosis, basal cell carcinoma and squamous cell carcinoma.

Other hyperproliferative cell disorders include blood vessel proliferation disorders, fibrotic disorders, autoimmune disorders, graft-versus-host rejection, tumors and cancers.

Blood vessel proliferative disorders include angiogenic and vasculogenic disorders. Proliferation of smooth muscle cells in the course of development of plaques in vascular tissue cause, for example, restenosis, retinopathies and atherosclerosis. Both cell migration and cell proliferation play a role in the formation of atherosclerotic lesions.

Fibrotic disorders are often due to the abnormal formation of an extracellular matrix. Examples of fibrotic disorders include hepatic cirrhosis and mesangial proliferative cell disorders. Hepatic cirrhosis is characterized by the increase in extracellular matrix constituents resulting in the formation of a hepatic scar. Hepatic cirrhosis can cause diseases such as cirrhosis of the liver. An increased extracellular matrix resulting in a hepatic scar can also be caused by viral infection such as hepatitis. Lipocytes appear to play a major role in hepatic cirrhosis.

Mesangial disorders are brought about by abnormal proliferation of mesangial cells. Mesangial hyperproliferative cell disorders include various human renal diseases, such as glomerulonephritis, diabetic nephropathy, malignant nephrosclerosis, thrombotic microangiopathy syndromes, transplant rejection, and glomerulopathies.

Another disease with a proliferative component is rheumatoid arthritis. Rheumatoid arthritis is generally considered an autoimmune disease that is thought to be associated with activity of autoreactive T cells, and to be caused by autoantibodies produced against collagen and IgE.

Other disorders that can include an abnormal cellular proliferative component include Bechet's syndrome, acute respiratory distress syndrome (ARDS), ischemic heart disease, post-dialysis syndrome, leukemia, acquired immune deficiency syndrome, vasculitis, lipid histiocytosis, septic shock and inflammation in general.

In certain embodiments, the condition is associated with an immune response.

Cutaneous contact hypersensitivity and asthma are just two examples of immune responses that can be associated with significant morbidity. Others include atopic dermatitis, eczema, Sjögren's Syndrome, including keratoconjunctivitis sicca secondary to Sjögren's Syndrome, alopecia areata, allergic responses due to arthropod bite reactions, Crohn's disease, aphthous ulcer, iritis, conjunctivitis, keratoconjunctivitis, ulcerative colitis, cutaneous lupus erythematosus, scleroderma, vaginitis, proctitis, and drug eruptions. These conditions may result in any one or more of the following symptoms or signs: itching, swelling, redness, blisters, crusting, ulceration, pain, scaling, cracking, hair loss, scarring, or oozing of fluid involving the skin, eye, or mucosal membranes.

In atopic dermatitis, and eczema in general, immunologically mediated leukocyte infiltration (particularly infiltration of mononuclear cells, lymphocytes, neutrophils, and eosinophils) into the skin importantly contributes to the pathogenesis of these diseases. Chronic eczema also is associated with significant hyperproliferation of the epidermis. Immunologically mediated leukocyte infiltration also occurs at sites other than the skin, such as in the airways in asthma and in the tear producing gland of the eye in keratoconjunctivitis sicca.

In other non-limiting embodiments, degraders of the present invention are used as topical agents in treating contact dermatitis, atopic dermatitis, eczematous dermatitis, psoriasis, Sjogren's Syndrome, including keratoconjunctivitis sicca secondary to Sjogren's Syndrome, alopecia areata, allergic responses due to arthropod bite reactions, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, and drug eruptions. The novel method may also be useful in reducing the infiltration of skin by malignant leukocytes in diseases such as mycosis fungoides. These compounds can also be used to treat an aqueous-deficient dry eye state (such as immune mediated keratoconjunctivitis) in a patient suffering therefrom, by administering the compound topically to the eye.

Exemplary cancers which may be treated by the present disclosed compounds either alone or in combination with at least one additional anti-cancer agent include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using the disclosed compounds according to the present invention include, for example, acute granulocytic leukemia, acute lymphocytic leukemia (ALL), acute myelogenous leukemia (AML), adenocarcinoma, adenosarcoma, adrenal cancer, adrenocortical carcinoma, anal cancer, anaplastic astrocytoma, angiosarcoma, appendix cancer, astrocytoma, Basal cell carcinoma, B-Cell lymphoma, bile duct cancer, bladder cancer, bone cancer, bone marrow cancer, bowel cancer, brain cancer, brain stem glioma, breast cancer, triple (estrogen, progesterone and HER-2) negative breast cancer, double negative breast cancer (two of estrogen, progesterone and HER-2 are negative), single negative (one of estrogen, progesterone and HER-2 is negative), estrogen-receptor positive, HER2-negative breast cancer, estrogen receptor-negative breast cancer, estrogen receptor positive breast cancer, metastatic breast cancer, luminal A breast cancer, luminal B breast cancer, Her2-negative breast cancer, HER2-positive or negative breast cancer, progesterone receptor-negative breast cancer, progesterone receptor-positive breast cancer, recurrent breast cancer, carcinoid tumors, cervical cancer, cholangiocarcinoma, chondrosarcoma, chronic lymphocytic leukemia (CLL), chronic myelogenous leukemia (CML), colon cancer, colorectal cancer, craniopharyngioma, cutaneous lymphoma, cutaneous melanoma, diffuse astrocytoma, ductal carcinoma in situ (DCIS), endometrial cancer, ependymoma, epithelioid sarcoma, esophageal cancer, ewing sarcoma, extrahepatic bile duct cancer, eye cancer, fallopian tube cancer, fibrosarcoma, gallbladder cancer, gastric cancer, gastrointestinal cancer, gastrointestinal carcinoid cancer, gastrointestinal stromal tumors (GIST), germ cell tumor glioblastoma multiforme (GBM), glioma, hairy cell leukemia, head and neck cancer, hemangioendothelioma, Hodgkin lymphoma, hypopharyngeal cancer, infiltrating ductal carcinoma (IDC), infiltrating lobular carcinoma (ILC), inflammatory breast cancer (IBC), intestinal Cancer, intrahepatic bile duct cancer, invasive/infiltrating breast cancer, Islet cell cancer, jaw cancer, Kaposi sarcoma, kidney cancer, laryngeal cancer, leiomyosarcoma, leptomeningeal metastases, leukemia, lip cancer, liposarcoma, liver cancer, lobular carcinoma in situ, low-grade astrocytoma, lung cancer, lymph node cancer, lymphoma, male breast cancer, medullary carcinoma, medulloblastoma, melanoma, meningioma, Merkel cell carcinoma, mesenchymal chondrosarcoma, mesenchymous, mesothelioma metastatic breast cancer, metastatic melanoma metastatic squamous neck cancer, mixed gliomas, monodermal teratoma, mouth cancer mucinous carcinoma, mucosal melanoma, multiple myeloma, Mycosis Fungoides, myelodysplastic syndrome, nasal cavity cancer, nasopharyngeal cancer, neck cancer, neuroblastoma, neuroendocrine tumors (NETs), non-Hodgkin's lymphoma, non-small cell lung cancer (NSCLC), oat cell cancer, ocular cancer, ocular melanoma, oligodendroglioma, oral cancer, oral cavity cancer, oropharyngeal cancer, osteogenic sarcoma, osteosarcoma, ovarian cancer, ovarian epithelial cancer ovarian germ cell tumor, ovarian primary peritoneal carcinoma, ovarian sex cord stromal tumor, Paget's disease, pancreatic cancer, papillary carcinoma, paranasal sinus cancer, parathyroid cancer, pelvic cancer, penile cancer, peripheral nerve cancer, peritoneal cancer, pharyngeal cancer, pheochromocytoma, pilocytic astrocytoma, pineal region tumor, pineoblastoma, pituitary gland cancer, primary central nervous system (CNS) lymphoma, prostate cancer, rectal cancer, renal cell carcinoma, renal pelvis cancer, rhabdomyosarcoma, salivary gland cancer, soft tissue sarcoma, bone sarcoma, sarcoma, sinus cancer, skin cancer, small cell lung cancer (SCLC), small intestine cancer, spinal cancer, spinal column cancer, spinal cord cancer, squamous cell carcinoma, stomach cancer, synovial sarcoma, T-cell lymphoma, testicular cancer, throat cancer, thymoma/thymic carcinoma, thyroid cancer, tongue cancer, tonsil cancer, transitional cell cancer, tubal cancer, tubular carcinoma, undiagnosed cancer, ureteral cancer, urethral cancer, uterine adenocarcinoma, uterine cancer, uterine sarcoma, vaginal cancer, vulvar cancer, T-cell lineage acute lymphoblastic leukemia (T-ALL), T-cell lineage lymphoblastic lymphoma (T-LL), peripheral T-cell lymphoma, Adult T-cell leukemia, Pre-B ALL, Pre-B lymphomas, large B-cell lymphoma, Burkitt's lymphoma, B-cell ALL, Philadelphia chromosome positive ALL, Philadelphia chromosome positive CML, juvenile myelomonocytic leukemia (JMML), acute promyelocytic leukemia (a subtype of AML), large granular lymphocytic leukemia, Adult T-cell chronic leukemia, diffuse large B cell lymphoma, follicular lymphoma; Mucosa-Associated Lymphatic Tissue lymphoma (MALT), small cell lymphocytic lymphoma, mediastinal large B cell lymphoma, nodal marginal zone B cell lymphoma (NMZL); splenic marginal zone lymphoma (SMZL); intravascular large B-cell lymphoma; primary effusion lymphoma; or lymphomatoid granulomatosis; B-cell prolymphocytic leukemia; splenic lymphoma/leukemia, unclassifiable, splenic diffuse red pulp small B-cell lymphoma; lymphoplasmacytic lymphoma; heavy chain diseases, for example, Alpha heavy chain disease, Gamma heavy chain disease, Mu heavy chain disease, plasma cell myeloma, solitary plasmacytoma of bone; extraosseous plasmacytoma; primary cutaneous follicle center lymphoma, T cell/histocyte rich large B-cell lymphoma, DLBCL associated with chronic inflammation; Epstein-Barr virus (EBV)+ DLBCL of the elderly; primary mediastinal (thymic) large B-cell lymphoma, primary cutaneous DLBCL, leg type, ALK+ large B-cell lymphoma, plasmablastic lymphoma; large B-cell lymphoma arising in HHV8-associated multicentric, Castleman disease; B-cell lymphoma, unclassifiable, with features intermediate between diffuse large B-cell lymphoma, or B-cell lymphoma, unclassifiable, with features intermediate between diffuse large B-cell lymphoma and classical Hodgkin lymphoma.

Nonlimiting general examples of disorders mediated by extracellular proteins also include, but are not limited to: AMD, macular edema, DME, diabetic retinopathy, mCNV; neurodegenerative disorders, metastatic colorectal cancer, non-squamous non-small-cell lung carcinoma, GMB, metastatic renal cell carcinoma, cervical cancer, AA amyloidosis, amyloid light chain (AL) amyloidosis, ankylosing spondylitis, antiphospholipid Ab syndrome, asthma, progression of parasite Schistosoma mansoni infection (IL-13), ATTR amyloidosis, Behcet syndrome, sepsis, inflammation, rheumatoid arthritis, atherosclerosis, ischemia/reperfusion injury; MGUS, Necrobiotic xanthogranuloma, JIA, psoriatic arthritis, plaque psoriasis, Crohn's disease, ulcerative colitis, Hidradenitis suppurativa uveitis; GvH disease; Castleman's disease, liver fibrosis, Still's Disease; cutaneous skin diseases including atopic dermatitis, transplant rejection, multiple myeloma, osteosclerotic multiple myeloma with peripheral neuropathy; pancreatic tumors; paraproteinemia (NR), prostate, gastric cancer; glioblastoma multiforme; acute coronary syndrome; hyperlipidemia (Rare/Broad), chronic urticaria, scleroderma, scleromyxedema, hereditary angioedema, clotting disorders, heparin-induced thrombocytopenia; Acquired Von Willebrand disease (AVWD), antiphospholipid antibody syndrome (APS or APLS); cryoglobulinemia; granulomatosis with polyangiitis (Wegener's)—sub-type of ANCA-associated vasculitis; idiopathic (Immune); thrombocytopenic purpura; IgG4-RD; Non-IgM MGUS; X-linked hypophosphatemia; Multiple System Atrophy (MSA), Parkinson's disease, Cachexia, Sarcopenia, Sporadic inclusion body myositis, muscular dystrophy, COPD; rhabdomyolysis; dialysis-related amyloidosis; focal segmental glomerulosclerosis (FSGS); IgA nephropathy (IgAN) and Henoch Schonlein Purpura (HSP); acute disseminated encephalomyelitis (ADEM); acute inflammatory demyelinating polyneuropathy (AIDP); Guillaine-Barre Syndrome; Alzheimer' disease & FTD; chronic inflammatory demyelinating polyneuropathy (CIDP); Creutzfeldt-Jakob disease (CJD); Huntington's disease; Miller Fisher Syndrome; Neuromyelitis optica spectrum disorder (NMOSD); Opsoclonus-myoclonus syndrome; PANDAS syndrome (pediatric autoimmune neuropsychiatric disorders associated with Streptococcal infections); Transverse myelitis; Emphysema, respiratory failure; Anthrax; Botulism; Sepsis; Staph. aureus toxic shock syndrome; Tetanus; Transplantation; Acromegaly; Cushing's disease; prion disease; secondary membranous nephropathy; and vasculitis.

### Disorders Mediated by Other Extracellular Proteins

In certain embodiments the extracellular protein degrading compound of the present invention degrades a protein other than immunoglobulin to treat a disorder described above. Non-limiting examples of disorders and Extracellular Proteins include:

### Cytokines/Chemokines

1) TNF-α mediates a number of disorders, including but not limited to rheumatoid arthritis, inflammatory bowel disease, graft-vs-host disease, ankylosing spondylitis, psoriasis, hidradenitis suppurativa, refractory asthma, systemic lupis erthyematosus, diabetes, and the induction of cachexia.
2) IL-2 mediates host versus graft rejection in transplants and autoimmune disorders, including, but not limited to, multiple sclerosis, idiopathic arthritis, iritis, anterior uveitis, IL-2 induced hypotension, psoriasis, and other autoimmune disorders
3) IL-1 mediates a number of auto-inflammatory and autoimmune disorders, including, but not limited to, Blau syndrome, cryopyrin-associated periodic syndromes, familial Mediterranean fever, Majeed syndrome; mevalonate kinase deficiency syndrome, pyogenic arthritis-pyoderma gangrenosum-acne syndrome, tumor necrosis factor receptor-associated periodic syndrome, Behçet's Disease, Sjögren's Syndrome, gout and chondrocalcinosis, periodic fever, aphthous stomatitis, pharyngitis, and cervical adenitis (or PFAPA) syndrome, rheumatoid arthritis, Type 2 diabetes mellitus, acute pericarditis, Chronic interstitial lung diseases (ILDs), and Still's disease amongst others.
4) IFN-γ mediates a wide range of autoimmune disorders, including, but not limited to rheumatoid arthritis, multiple sclerosis (MS), corneal transplant rejection, and various autoimmune skin diseases such as psoriasis, alopecia areata, vitiligo, acne vulgaris, and others.
5) IL-21 mediates a number of autoimmune disorders, including Sjögren's syndrome, systemic lupus erythematosus, type 1 diabetes, multiple sclerosis, rheumatoid arthritis, and inflammatory bowel disease.
6) IL-22 mediates a number of autoimmune disorders, including, but not limited to, graft versus host disease (GVHD), psoriasis, rheumatoid arthritis, atopic dermatitis, and asthma.
7) IL-10 has been implicated in tumor survival and protection against cytotoxic chemotherapeutic drugs.
8) IL-5 has been implicated in a number of allergic disorders, including, but not limited to, asthma, nasal polyposis, atopic dermatitis, eosinophilic esophagitis, hypereosinophilic syndrome, and Churg-Strauss syndrome.
9) IL-6 has been implicated in a number of inflammatory diseases and cancers, including, but not limited to, Castleman's disease, metastatic castration-associated prostate cancer, renal cell carcinoma, large-cell lung carcinoma, ovarian cancer, rheumatoid arthritis, asthma.
10) IL-8 has been implicated in the promotion of tumor progression, immune escape, epithelial-mesenchymal transition, and recruitment of myeloid-derived suppressor cells. Studies have demonstrated that high serum IL-8 levels correlate with poor prognosis in many malignant tumors. Preclinical studies have shown that IL-8 blockade may reduce mesenchymal features in tumor cells, making them less resistant to treatment.
11) C-C motif chemokine ligand 2 (CCL2) has been implicated in the recruitment of monocytes into the arterial wall during the disease process of atherosclerosis.
12) Macrophage Migration Inhibitory Factor (MIF) is a mediator of tumor progression; systemic inflammation; atherosclerosis; rheumatoid arthritis; and systemic lupus erythematosus, among others.

### Growth Factors

1) Fibroblast Growth Factor 1 (FGF1) can induce angiogenesis. FGF1 has been implicated in oncogenesis, cancer cell proliferation, resistance to anticancer therapies, and neoangiogenesis.
2) Fibroblast Growth Factor 2 (FGF2) has been implicated in oncogenesis, cancer cell proliferation, resistance to anticancer therapies, and neoangiogenesis.
3) Vascular Epithelial Growth Factor (VEGF-A) has been implicated in the vascularization and angiogenesis of tumors.
4) Transforming Growth Factor-β1(TGF-β1) expression in the tumor microenvironment has been associated with a poor prognosis, and is implicated in TGF-β1 mediated tumor suppression via T-cell exclusion. TGF-β1 expression has also been implicated in hematological malignancies and fibrosis.
5) Transforming Growth Factor-β2 (TGF-β2) expression in the tumor microenvironment has been associated with a poor prognosis, and is implicated in TGF-β2 mediated tumor suppression via T-cell exclusion. TGF-β2 expression has also been implicated in hematological malignancies and fibrosis.
6) Placental Growth Factor (PGF) promotes cell tumor growth, and has been implicated in age-related macular degeneration (AMD) and choroidal neovascularization (CNV).

### Esterase

1) Cholinesterase has been implicated in cognitive disorders such as dementia and Alzheimer's disease.

### Coagulation Factors

1) Carboxypeptidase B2 has been implicated and targeted to inhibit thrombosis.
2) Coagulation Factor Xa is a mediator in the development of deep vein thrombosis and acute pulmonary embolism, and the risk of stroke and embolism in people with nonvalvular atrial fibrillation.
3) Coagulation Factor XI is a mediator in the development of deep vein thrombosis and acute pulmonary embolism, and the risk of stroke and embolism in people with nonvalvular atrial fibrillation.
4) Coagulation Factor XII has been implicated in the development of deep vein thrombosis and acute pulmonary embolism, and the risk of stroke and embolism in people with nonvalvular atrial fibrillation.
5) Coagulation Factor XIII has been implicated in the development of deep vein thrombosis and acute pulmonary embolism, and the risk of stroke and embolism in people with nonvalvular atrial fibrillation.
6) Prothrombin is involved in blood clot formation and arterial and venous thrombosis, and thromboembolism associated with atrial fibrillation.
7) Coagulation Factor VII is involved in blood clot formation and arterial and venous thrombosis, and thromboembolism associated with atrial fibrillation.
8) Coagulation Factor IX is involved in blood clot formation and arterial and venous thrombosis, and thromboembolism associated with atrial fibrillation.

### Extracellular Matrix Proteins

1) Neutrophil Elastase—Neutrophil elastase has been implicated in a number of disorders, including lung disease, chronic obstructive pulmonary disease, pneumonia, respiratory distress, and acute lung injury (ALI), and cystic fibrosis, as well as chronic kidney disease.
2) Fibronectin-1—Interfering with FN polymerization may attenuate myofibroblasts and fibrosis and improve cardiac function after ischemia/reperfusion (I/R) injury.
3) Thrombospondin-1—TSP-1 has been implicated in a number of diseases, including in promoting certain cancers such as breast cancer, prostate cancer, melanoma, SCLC, osteosarcoma, cutaneous squamous cell carcinoma, oral squamous cell carcinoma, papillary thyroid carcinoma, thyroid cancer, medulloblastoma, and fibrotic disorders such as diabetes, liver fibrosis, and in multiple myeloma.
4) Urokinase-type Plasminogen Activator (UPA)—UPA has been implicated in vascular diseases and cancer progression. Elevated expression levels of urokinase and several other components of the plasminogen activation system are found to be correlated with tumor malignancy.
5) Plasminogen Activator, Tissue Type (TPA)—PLA has been shown activated in various cancers including oral malignancy.
6) Plasminogen (PLG)—PLG has been implicated in tumor invasion and inflammation.
7) Plasminogen Activator Inhibitor-1 (PAI-1)—PAI-1 has been implicated in angiogenesis, metastasis, and poor prognosis in tumors, including, but not limited to, oral cancers and breast cancers.

### Peptidase

1) Kallikrein-1—Kallikrein has been implicated in adverse reactions in hereditary angioedema (HAE).
2) Plasma Kallikrein—Plasma kallikrein has been implicated in retinal dysfunction, the development of diabetic macular edema and hereditary angioedema (HAE).
3) Matrix Metallopeptidase-1—MMP-1 has been implicated in cardiovascular disease, development of fibrosis, and growth of certain cancers such as bladder cancer.
4) Phospholipase A2, Group IIA (PA2GA)—PA2GA has been implicated in a number of diseases, including cardiovascular diseases, atherosclerosis, immune disorders, and cancer.

### Lipase

1) Lipoprotein Lipase—Lipoprotein lipase has been implicated in the development of cardiovascular disease and obesity.
2) Phospholipase A2, Group IB (PA21B)—PA21B has been implicated in a number of diseases, including cardiovascular diseases, atherosclerosis, immune disorders and cancer.

### Convertase

1) Proprotein Convertase Subtilisin/Kexin Type 9 (PCSK-9)—PCSK-9 has been implicated in high blood cholesterol and the development of cardiovascular disease.

Certain targeted extracellular proteins include but are not limited to: SAA (serum amyloid A), amyloid light chains, antibodies to Klebsiella dipeptidase protein; Ig antibodies to anionic phospholipids and beta-2-glycoprotein-I; IL-13; MIF; Transthyretin (misfolded), IgG autoantibodies to thyroid peroxidase, thyroglobulin and TSH receptors; TNF-α; Protein arginine deiminase (PAD, PAD4); antibodies to citrullinated protein antibody (ACPA); anti-DNA antibodies; IL-17; Lysyl Oxidase 2 (LOXL2); IL-18; Blys; B cell activating factor (BAFF); CD40 (soluble); CXCL12; soluble PSMA; matrix metalloproteinase IX (MMP-9); hormone-sensitive lipase; lipoprotein-associated phospholipase A2; Factor Xa; DPP4; thrombin; PCSK9; ApoB-100; Complement component C3b; PKK (pre-kallikrein); Factor XI; PF4; Anti-vWF antibodies; anticardiolipin antibodies and lupus anticoagulant; FGF23 (fibroblast growth factor 23); Plasminogen activator inhibitor type 1 (PAI-1); Myeloperoxidase (MPO) extracellular; Myostatin; Beta2-m; suPAR (soluble urokinase plasminogen activator receptor); anti-ganglioside IgG; amyloid beta; Tau; CJD-associate prion; anti-ganglioside IgG; HTT; anti-ganglioside IgG; synuclein; elastase; PABA (protective antigen of Bacillus anthracis); edema factor; Botulinum toxin; C. difficile toxin B; hemolysin; tetanus toxin; IL-2; growth hormone and ACTH.

### V. Combination Treatment

In certain aspects, a treatment regimen is provided comprising the administration of an effective amount of an extracellular protein degrader of the present invention or a pharmaceutically acceptable composition, salt, isotopic analog, or prodrug thereof in combination or in alternation with at least one additional therapeutic agent for the treatment of a disorder mediated by the Target Extracellular Protein, for example an immunoglobulin. The combinations and/or alternations disclosed herein can be administered for beneficial, additive, or synergistic effect in the treatment of extracellular protein mediated disorders.

In certain embodiments, a treatment regimen is provided for the treatment of IgA nephropathy comprising the administration of an immunoglobulin degrader of the present invention or a pharmaceutically acceptable composition, salt, isotopic analog, or prodrug thereof in combination or in alternation with at least one additional therapeutic agent. In certain embodiments, at least one of the additional therapeutic agents is an ACE inhibitor. In certain embodiments, at least one of the additional therapeutic agents is an AT1R antagonist. In certain embodiments, at least one of the additional therapeutic agents is an angiotensin receptor blocker. In certain embodiments, at least one of the additional therapeutic agents is an omega-3 fatty acid. In certain embodiments, at least one of the additional therapeutic agents is a corticosteroid. In certain embodiments, at least one of the additional therapeutic agents is a statin. In certain embodiments, at least one of the additional therapeutic agents is a diuretic. In certain embodiments, at least one of the additional therapeutic agents is an immunosuppressant. In certain embodiments, at least one of the additional therapeutic agents is selected from AVB-S6-500, iptacopan, atacicept, rituximab, BION-1301, mycophenolic acid, mycophenolate mofetil, allopurinol, blisibimod, bortezomib, paricalcitol, tacrolimus, aliskiren, enalapril, cemdisiran, irbesartan, rapamycin, calcitriol, and ravulizumab.

In certain embodiments, a treatment regimen is provided for the treatment of celiac disease comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a steroid. In certain embodiments, at least one of the additional therapeutic agents is a copper supplement. In certain embodiments, at least one of the additional therapeutic agents is a zinc supplement. In certain embodiments, at least one of the additional therapeutic agents is an iron supplement. In certain embodiments, at least one of the additional therapeutic agents is a folate supplement. In certain embodiments, at least one of the additional therapeutic agents is a vitamin B12 supplement. In certain embodiments, at least one of the additional therapeutic agents is a vitamin D supplement. In certain embodiments, at least one of the additional therapeutic agents is a vitamin K supplement. In certain embodiments, at least one of the additional therapeutic agents is selected from azathioprine, budesonide, and dapsone.

In certain embodiments, a treatment regimen is provided for the treatment of IgA vasculitis comprising the administration of an immunoglobulin degrader of the present invention or a pharmaceutically acceptable composition, salt, isotopic analog, or prodrug thereof in combination or in alternation with at least one additional therapeutic agent. In certain embodiments, at least one of the additional therapeutic agents is a steroid. In certain embodiments, at least one of the additional therapeutic agents is an anti-inflammatory.

In certain embodiments, a treatment regimen is provided for the treatment of IgA pemphigus comprising the administration of an immunoglobulin degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is a retinoid. In certain embodiments, at least one of the additional therapeutic agents is selected from dapsone, colchicine, mycophenolate mofetil, and adalimumab.

In certain embodiments, a treatment regimen is provided for the treatment of dermatitis herpetiformis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a steroid. In certain embodiments, at least one of the additional therapeutic agents is an immunosuppressant. In certain embodiments, at least one of the additional therapeutic agents is a tetracycline. In certain embodiments, at least one of the additional therapeutic agents is selected from dapsone, sulfasalazine, sulphapyridine, sulfamethoxypyridazine, cyclosporin A, azathioprine, colchicine, heparin, nicotinamide, mycophenolate, and rituximab.

In certain embodiments, a treatment regimen is provided for the treatment of inflammatory bowel disease comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is an aminosalicylate. In certain embodiments, at least one of the additional therapeutic agents is an immunosuppressant. In certain embodiments, at least one of the additional therapeutic agents is selected from mesalamine, balsalazide, olsalazine, azathioprine, mercaptopurine, methotrexate, infliximab, adalimumab, golimumab, certolizumab, vedolizumab, and ustekinumab.

In certain embodiments, a treatment regimen is provided for the treatment of Sjögren's syndrome comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a non-steroidal anti-inflammatory drug. In certain embodiments, at least one of the additional therapeutic agents is an immunosuppressant. In certain embodiments, at least one of the additional therapeutic agents is selected from cyclosporine, lifitegrast, pilocarpine, cevimeline, hydroxychloroquine, and methotrexate.

In certain embodiments, a treatment regimen is provided for the treatment of ankylosing spondylitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a non-steroidal anti-inflammatory drug. In certain embodiments, at least one of the additional therapeutic agents is a TNF blocker.

In certain embodiments, at least one of the additional therapeutic agents is an IL-17 inhibitor. In certain embodiments, at least one of the additional therapeutic agents is selected from naproxen, indomethacin, adalimumab, certolizumab pegol, etanercept, golimumab, infliximab, secukinumab, ixekizumab, and tofacitinib.

In certain embodiments, a treatment regimen is provided for the treatment of alcoholic liver cirrhosis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a glucocorticosteroid. In certain embodiments, at least one of the additional therapeutic agents is an antioxidant. In certain embodiments, at least one of the additional therapeutic agents is selected from pentoxifylline and infliximab.

In certain embodiments, a treatment regimen is provided for the treatment of acquired immunodeficiency syndrome comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a non-nucleoside reverse transcriptase inhibitor. In certain embodiments, at least one of the additional therapeutic agents is a nucleoside or nucleotide reverse transcriptase inhibitor. In certain embodiments, at least one of the additional therapeutic agents is a protease inhibitor. In certain embodiments, at least one of the additional therapeutic agents is an integrase inhibitor. In certain embodiments, at least one of the additional therapeutic agents is an entry or fusion inhibitor. In certain embodiments, at least one of the additional therapeutic agents is selected from efavirenz, rilpivirine, doravirine, abacavir, tenofovir, emtricitabine, lamivudine, zidovudine, atazanavir, darunavir, lopinavir, ritonavir, bictegravir, raltegravir, dolutegravir, enfuvirtide, and maraviroc.

In certain embodiments, a treatment regimen is provided for the treatment of IgA multiple myeloma comprising the administration of an immunoglobulin degrader of the present invention or a pharmaceutically acceptable composition, salt, isotopic analog, or prodrug thereof in combination or in alternation with at least one additional therapeutic agent. In certain embodiments, at least one of the additional therapeutic agents is an HDAC inhibitor. In certain embodiments, at least one of the additional therapeutic agents is an immunomodulatory drug. In certain embodiments, at least one of the additional therapeutic agents is a steroid. In certain embodiments, at least one of the additional therapeutic agents is an antibody drug. In certain embodiments, at least one of the additional therapeutic agents is a BCL2 inhibitor. In certain embodiments, at least one of the additional therapeutic agents is a proteasome inhibitor. In certain embodiments, at least one of the additional therapeutic agents is a selective inhibitor of nuclear export. In certain embodiments, at least one of the additional therapeutic agents is selected from prednisone, cyclophosphamide, melphalan, vincristine, doxorubicin, dexamethasone, thalidomide, bortezomib, lenalidomide, carfilzomib, pomalidomide, daratumumab, Panobinostat, elotuzumab, ixazomib, isatuximab, venetoclax, marizomib, oprozomib, and Selinexor.

In certain embodiments, a treatment regimen is provided for the treatment of IgA heavy chain disease comprising the administration of an immunoglobulin degrader of the present invention or a pharmaceutically acceptable composition, salt, isotopic analog, or prodrug thereof in combination or in alternation with at least one additional therapeutic agent. In certain embodiments, at least one of the additional therapeutic agents is an antibiotic. In certain embodiments, at least one of the additional therapeutic agents is a chemotherapeutic drug. In certain embodiments, at least one of the additional therapeutic agents is a steroid. In certain embodiments, at least one of the additional therapeutic agents is selected from doxorubicin, cyclophosphamide, vincristine, prednisone, teniposide, bleomycin, vinblastine, procarbazine, and prednisolone.

In certain embodiments, a treatment regimen is provided for the treatment of linear IgA bullous dermatosis comprising the administration of an immunoglobulin degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is a tetracycline. In certain embodiments, at least one of the additional therapeutic agents is an immunoglobulin. In certain embodiments, at least one of the additional therapeutic agents is selected from erythromycin, sulphapyridine, colchicine, and mycophenolate mofetil.

In certain embodiments, a treatment regimen is provided for the treatment of monoclonal gammopathy of undetermined significance (MGUS) comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is selected from alendronate, risedronate, ibandronate, and zoledronic acid.

In certain embodiments, a treatment regimen is provided for the treatment of systemic fibroinflammatory disease comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a glucocorticoid. In certain embodiments, at least one of the additional therapeutic agents is a calcineurin inhibitor. In certain embodiments, at least one of the additional therapeutic agents is selected from prednisolone, methotrexate, azathioprine, mycophenolate, mycophenolate mofetil, 6-mecaptopurine, cyclophosphamide, and rituximab.

In certain embodiments, a treatment regimen is provided for the treatment of Riedel's thyroiditis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a glucocorticoid. In certain embodiments, at least one of the additional therapeutic agents is an immunosuppressant. In certain embodiments, at least one of the additional therapeutic agents is selected from tamoxifen, prednisone, and mycophenolate mofetil.

In certain embodiments, a treatment regimen is provided for the treatment of inflammatory pseudotumors comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is a cyclooxygenase 2 inhibitor. In certain embodiments, at least one of the additional therapeutic agents is an anti-inflammatory. In certain embodiments, at least one of the additional therapeutic agents is thalidomide.

In certain embodiments, a treatment regimen is provided for the treatment of mediastinal fibrosis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is rituximab.

In certain embodiments, a treatment regimen is provided for the treatment of retroperitoneal fibrosis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is an immunosuppressant. In certain embodiments, at least one of the additional therapeutic agents is selected from mycophenolate mofetil, methotrexate, azathioprine, cyclophosphamide, tamoxifen, prednisone, and cyclosporin A.

In certain embodiments, a treatment regimen is provided for the treatment of aortitis or periaortitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is glucocorticoid. In certain embodiments, at least one of the additional therapeutic agents is an immunosuppressant. In certain embodiments, at least one of the additional therapeutic agents is selected from prednisone, methotrexate, tocilizumab, cyclophosphamide, azathioprine, and cyclosporin A.

In certain embodiments, a treatment regimen is provided for the treatment of proximal biliary strictures comprising the administration of an extracellular protein degrader of the present invention or a pharmaceutically acceptable composition, salt, isotopic analog, or prodrug thereof in combination or in alternation with at least one additional therapeutic agent.

In certain embodiments, a treatment regimen is provided for the treatment of gastroenteritis comprising the administration of an extracellular protein degrader of the present invention or a pharmaceutically acceptable composition, salt, isotopic analog, or prodrug thereof in combination or in alternation with at least one additional therapeutic agent.

In certain embodiments, a treatment regimen is provided for the treatment of IgA monoclonal gammopathy comprising the administration of an immunoglobulin degrader of the present invention or a pharmaceutically acceptable composition, salt, isotopic analog, or prodrug thereof in combination or in alternation with at least one additional therapeutic agent.

In certain embodiments, a treatment regimen is provided for the treatment of IgG monoclonal gammopathy comprising the administration of an immunoglobulin degrader of the present invention or a pharmaceutically acceptable composition, salt, isotopic analog, or prodrug thereof in combination or in alternation with at least one additional therapeutic agent.

In certain embodiments, a treatment regimen is provided for the treatment of IgE monoclonal gammopathy comprising the administration of an immunoglobulin degrader of the present invention or a pharmaceutically acceptable composition, salt, isotopic analog, or prodrug thereof in combination or in alternation with at least one additional therapeutic agent.

In certain embodiments, a treatment regimen is provided for the treatment of idiopathic hypocomplementemic tubulointerstitial nephritis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a steroid. In certain embodiments, at least one of the additional therapeutic agents is selected from prednisone, rituximab, and mycophenolic acid.

In certain embodiments, a treatment regimen is provided for the treatment of multifocal fibrosclerosis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an immunosuppressant. In certain embodiments, at least one of the additional therapeutic agents is selected from cyclosporine and prednisolone.

In certain embodiments, a treatment regimen is provided for the treatment of pachymeningitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is selected from prednisone, azathioprine, cyclophosphamide, methotrexate, and rituximab.

In certain embodiments, a treatment regimen is provided for the treatment of pancreatic enlargement comprising the administration of an extracellular protein degrader of the present invention or a pharmaceutically acceptable composition, salt, isotopic analog, or prodrug thereof in combination or in alternation with at least one additional therapeutic agent.

In certain embodiments, a treatment regimen is provided for the treatment of tumefactive lesions comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is an immunosuppressant. In certain embodiments, at least one of the additional therapeutic agents is selected from rituximab and cyclophosphamide.

In certain embodiments, a treatment regimen is provided for the treatment of rheumatoid arthritis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is a non-steroidal anti-inflammatory. In certain embodiments, at least one of the additional therapeutic agents is a disease-modifying antirheumatic drug. In certain embodiments, at least one of the additional therapeutic agents is a biologic drug. In certain embodiments, at least one of the additional therapeutic agents is a Janus-associated kinase inhibitor. In certain embodiments, at least one of the additional therapeutic agents is an opioid. In certain embodiments, at least one of the additional therapeutic agents is selected from ibuprofen, naproxen sodium, prednisone, methotrexate, leflunomide, hydroxychloroquine, sulfasalazine, abatacept, adalimumab, anakinra, baricitinib, certolizumab, etanercept, golimumab, infliximab, rituximab, sarilumab, tocilizumab, tofacitinib, minocycline, celecoxib, nabumetone, piroxicam, diclofenac, diflunisal, indomethacin, ketoprofen, etodolac, fenoprofen, flurbiprofen, ketorolac, meclofenamate, mefenamic acid, meloxicam, oxaprozin, sulindac, salsalate, tolmetin, codeine, fentanyl, hydrocodone, hydromorphone, morphine, meperidine, oxycodone, tramadol, betamethasone, dexamethasone, cortisone, hydrocortisone, methylprednisolone, and prednisolone.

In certain embodiments, a treatment regimen is provided for the treatment of multiple sclerosis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is an interferon beta drug. In certain embodiments, at least one of the additional therapeutic agents is a biologic drug. In certain embodiments, at least one of the additional therapeutic agents is a muscle relaxant. In certain embodiments, at least one of the additional therapeutic agents is selected from prednisone, methylprednisolone, ocrelizumab, glatiramer acetate, interferon beta-la, interferon beta-1b, fingolimod, dimethyl fumarate, diroximel fumarate, teriflunomide, siponimod, cladribine, ocrelizumab, natalizumab, alemtuzumab, baclofen, tizanidine, cyclobenzaprine, amantadine, modafinil, methylphenidate, and dalfampridine.

In certain embodiments, a treatment regimen is provided for the treatment of myasthenic gravis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is a cholinesterase inhibitor. In certain embodiments, at least one of the additional therapeutic agents is an immunosuppressant. In certain embodiments, at least one of the additional therapeutic agents is a monoclonal antibody. In certain embodiments, at least one of the additional therapeutic agents is selected from pyridostigmine, neostigmine, azathioprine, mycophenolate mofetil, cyclosporine, methotrexate, tacrolimus, rituximab, and eculizumab.

In certain embodiments, a treatment regimen is provided for the treatment of psoriatic arthritis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a non-steroidal anti-inflammatory.

In certain embodiments, at least one of the additional therapeutic agents is a disease-modifying antirheumatic drug. In certain embodiments, at least one of the additional therapeutic agents is an immunosuppressant. In certain embodiments, at least one of the additional therapeutic agents is a biologic drug. In certain embodiments, at least one of the additional therapeutic agents is selected from apremilast, ibuprofen, naproxen sodium, methotrexate, leflunomide, sulfasalazine, azathioprine, cyclosporine, abatacept, adalimumab, certolizumab, etanercept, golimumab, infliximab, ixekizumab, secukinumab, tofacitinib, and ustekinumab.

In certain embodiments, a treatment regimen is provided for the treatment of systemic lupus erythematosus (SLE) comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a non-steroidal anti-inflammatory. In certain embodiments, at least one of the additional therapeutic agents is an antimalarial drug. In certain embodiments, at least one of the additional therapeutic agents is a corticosteroid. In certain embodiments, at least one of the additional therapeutic agents is an immunosuppressant. In certain embodiments, at least one of the additional therapeutic agents is a biologic drug. In certain embodiments, at least one of the additional therapeutic agents is selected from voclosporin, abatacept, anifrolumab, naproxen sodium, ibuprofen, hydroxychloroquine, methylprednisolone, azathioprine, mycophenolate, methotrexate, cyclosporine, leflunomide, belimumab, and rituximab.

In certain embodiments, a treatment regimen is provided for the treatment of sclerosing cholangitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a bile acid sequestrant. In certain embodiments, at least one of the additional therapeutic agents is an antibiotic. In certain embodiments, at least one of the additional therapeutic agents is an antihistamine. In certain embodiments, at least one of the additional therapeutic agents is an opioid antagonist. In certain embodiments, at least one of the additional therapeutic agents is selected from ursodeoxycholic acid, rifampin, naltrexone, cholestyramine, colestipol, and colesevelam.

In certain embodiments, a treatment regimen is provided for the treatment of atopic asthma comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a bronchodilator. In certain embodiments, at least one of the additional therapeutic agents is a corticosteroid. In certain embodiments, at least one of the additional therapeutic agents is a leukotriene modifier. In certain embodiments, at least one of the additional therapeutic agents is a beta agonist. In certain embodiments, at least one of the additional therapeutic agents is selected from omalizumab, fluticasone, budesonide, mometasone, ciclesonide, montelukast, zafirlukast, zileuton, salmeterol, and formoterol.

In certain embodiments, a treatment regimen is provided for the treatment of cutaneous contact hypersensitivity comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a corticosteroid.

In certain embodiments, a treatment regimen is provided for the treatment of allergic conjunctivitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a steroid. In certain embodiments, at least one of the additional therapeutic agents is cromolyn.

In certain embodiments, a treatment regimen is provided for the treatment of allergic urticaria comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a histamine blocker. In certain embodiments, at least one of the additional therapeutic agents is an antidepressant. In certain embodiments, at least one of the additional therapeutic agents is a monoclonal antibody. In certain embodiments, at least one of the additional therapeutic agents is an immunosuppressant. In certain embodiments, at least one of the additional therapeutic agents is a corticosteroid. In certain embodiments, at least one of the additional therapeutic agents is selected from tacrolimus, cyclosporine, omalizumab, zafirlukast, montelukast, doxepin, prednisone, cimetidine, famotidine, loratadine, fexofenadine, cetirizine, and desloratadine.

In certain embodiments, a treatment regimen is provided for the treatment of anaphylactic shock comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a beta-agonist. In certain embodiments, at least one of the additional therapeutic agents is selected from epinephrine, cortisone, and albuterol.

In certain embodiments, a treatment regimen is provided for the treatment of nasal polyposis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a corticosteroid. In certain embodiments, at least one of the additional therapeutic agents is selected from dupilumab, prednisone, fluticasone, budesonide, mometasone, triamcinolone, beclomethasone, and ciclesonide.

In certain embodiments, a treatment regimen is provided for the treatment of keratoconjunctivitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a mast cell stabilizer. In certain embodiments, at least one of the additional therapeutic agents is a non-steroidal anti-inflammatory. In certain embodiments, at least one of the additional therapeutic agents is a corticosteroid. In certain embodiments, at least one of the additional therapeutic agents is an antiviral. In certain embodiments, at least one of the additional therapeutic agents is selected from cidofovir, ganciclovir, cromolyn sodium, nedocromil, sodium, lodoxamide, cyclosporine A, tacrolimus, lifitegrast, doxycycline, and vitamin A.

In certain embodiments, a treatment regimen is provided for the treatment of mastocytosis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a leukotriene modifier. In certain embodiments, at least one of the additional therapeutic agents is a mast cell stabilizer. In certain embodiments, at least one of the additional therapeutic agents is a corticosteroid. In certain embodiments, at least one of the additional therapeutic agents is a chemotherapeutic drug.

In certain embodiments, a treatment regimen is provided for the treatment of eosinophilic gastrointestinal disease comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a proton pump inhibitor. In certain embodiments, at least one of the additional therapeutic agents is a corticosteroid. In certain embodiments, at least one of the additional therapeutic agents is an immunomodulator. In certain embodiments, at least one of the additional therapeutic agents is a biologic drug. In certain embodiments, at least one of the additional therapeutic agents is a leukotriene D4 receptor antagonist. In certain embodiments, at least one of the additional therapeutic agents is selected from montelukast, cromolyn sodium, omeprazole, lansoprazole, dexlansoprazole, esomeprazole, pantoprazole, rabeprazole, ilaprazole, fluticasone propionate, budesonide, prednisone, azathioprine, 6-mercaptopurine, timapiprant, mepolizumab, dectrekumab, and cendakimab.

In certain embodiments, a treatment regimen is provided for the treatment of bullous pemphigoid comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is an immunosuppressant. In certain embodiments, at least one of the additional therapeutic agents is selected from azathioprine, mycophenolate mofetil, rituximab, methotrexate, and tetracycline.

In certain embodiments, a treatment regimen is provided for the treatment of chemotherapy induced hypersensitivity reaction comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a histamine 1 antagonist. In certain embodiments, at least one of the additional therapeutic agents is a histamine 2 antagonist. In certain embodiments, at least one of the additional therapeutic agents is a corticosteroid. In certain embodiments, at least one of the additional therapeutic agents is an anticonvulsant.

In certain embodiments, a treatment regimen is provided for the treatment of seasonal allergic rhinitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is a decongestant. In certain embodiments, at least one of the additional therapeutic agents is a corticosteroid. In certain embodiments, at least one of the additional therapeutic agents is a leukotriene modifier. In certain embodiments, at least one of the additional therapeutic agents is selected from fexofenadine, diphenhydramine, desloratadine, loratadine, levocetirizine, cetirizine, oxymetazoline, pseudoephedrine, phenylephrine, cromolyn sodium, montelukast, and ipratropium.

In certain embodiments, a treatment regimen is provided for the treatment of interstitial cystitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a non-steroidal anti-inflammatory. In certain embodiments, at least one of the additional therapeutic agents is a tricyclic antidepressant. In certain embodiments, at least one of the additional therapeutic agents is an antihistamine. In certain embodiments, at least one of the additional therapeutic agents is selected from pentosan polysulfate sodium, loratadine, amitriptyline, imipramine, ibuprofen, naproxen sodium, dimethyl sulfoxide, and heparin.

In certain embodiments, a treatment regimen is provided for the treatment of eosinophilic esophagitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a proton pump inhibitor. In certain embodiments, at least one of the additional therapeutic agents is a corticosteroid. In certain embodiments, at least one of the additional therapeutic agents is an immunomodulator. In certain embodiments, at least one of the additional therapeutic agents is a biologic drug. In certain embodiments, at least one of the additional therapeutic agents is a leukotriene D4 receptor antagonist. In certain embodiments, at least one of the additional therapeutic agents is selected from montelukast, cromolyn sodium, omeprazole, lansoprazole, dexlansoprazole, esomeprazole, pantoprazole, rabeprazole, ilaprazole, fluticasone propionate, budesonide, prednisone, azathioprine, 6-mercaptopurine, timapiprant, mepolizumab, dectrekumab, and cendakimab.

In certain embodiments, a treatment regimen is provided for the treatment of angioedema comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is an antihistamine. In certain embodiments, at least one of the additional therapeutic agents is an immunosuppressant. In certain embodiments, at least one of the additional therapeutic agents is a corticosteroid.

In certain embodiments, a treatment regimen is provided for the treatment of acute interstitial nephritis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is selected from cyclophosphamide, cyclosporine, and mycophenolate mofetil.

In certain embodiments, a treatment regimen is provided for the treatment of atopic eczema comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is a calcineurin inhibitor. In certain embodiments, at least one of the additional therapeutic agents is a biologic drug. In certain embodiments, at least one of the additional therapeutic agents is selected from dupilumab, prednisone, tacrolimus, and pimecrolimus.

In certain embodiments, a treatment regimen is provided for the treatment of eosinophilic bronchitis comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is a leukotriene receptor antagonist. In certain embodiments, at least one of the additional therapeutic agents is selected from budesonide, fluticasone, and montelukast.

In certain embodiments, a treatment regimen is provided for the treatment of chronic obstructive pulmonary disease comprising the administration of an extracellular protein degrader of the present invention or a pharmaceutically acceptable composition, salt, isotopic analog, or prodrug thereof in combination or in alternation with at least one additional therapeutic agent. In certain embodiments, at least one of the additional therapeutic agents is a bronchodilator. In certain embodiments, at least one of the additional therapeutic agents is a corticosteroid. In certain embodiments, at least one of the additional therapeutic agents is a phosphodiesterase-4 inhibitor. In certain embodiments, at least one of the additional therapeutic agents is an antibiotic. In certain embodiments, at least one of the additional therapeutic agents is selected from theophylline, azithromycin, roflumilast, fluticasone, budesonide, albuterol, ipratropium, levalbuterol, aclidinium, arformoterol, formoterol, indacaterol, tiotropium, salmeterol, and umeclidinium.

In certain embodiments, a treatment regimen is provided for the treatment of hyper-IgE syndrome (Job's syndrome) comprising the administration of an immunoglobulin degrader of the present invention or a pharmaceutically acceptable composition, salt, isotopic analog, or prodrug thereof in combination or in alternation with at least one additional therapeutic agent. In certain embodiments, at least one of the additional therapeutic agents is a corticosteroid. In certain embodiments, at least one of the additional therapeutic agents is an antibiotic.

### VI. Biological Activity

### Example 1. Binding Affinity of ASGPR Ligands Measured Using Surface Plasmon Resonance (SPR)

The dissociation constants (Kd) of compounds described herein to the ASGP receptor were measured by surface plasmon resonance using a Biacore 8K instrument (GE Healthcare) at 25° C.

Biotinylated ASGPR (20 μg/ml) was immobilized on SA sensor chips (GE Healthcare) at flow rate of 5 μl/min for 90 sec reaching an immobilization level ranging from 500-3000 resonance units (RU). The running buffer was 50 mM HEPES, pH7.5, 150 mM NaCl, 50 mM CaCl₂), 0.01% P20, 3% DMSO. The concentration of compounds vary from 2 mM to 5 nM depending on Kd values. The compounds are diluted 2 or 3-fold with 8 concentration points. Solutions containing serially diluted compounds are injected at a flow rate of 40 L/min for 150-300 sec followed by a 90-300 sec dissociation phase for each concentration. The surface was regenerated using 50 mM HEPES, pH 7.5, 150 mM NaCl, 5 mM EDTA, 0.01% P20, 3% DMSO.

Data was processed using the analysis software in Biacore 8K to perform background subtraction, double referencing, and solvent correction. Values of affinity expressed as the dissociation constants (Kd) were determined by fitting the steady state binding responses (RUs) as a function of the concentration ([Compound]) using the following equation: RU=RUmax/(KD+[Compound]), where RU max is the calculated maximal response. Where appropriate double-referenced data were fit to using a 1:1 binding model for kinetic analysis. Obtained data is summarized in Table 1 below.

TABLE 1A
ASGPR Binding of Select Compounds

| Com- | | |
|---|---|---|
| pound | | ASGPR |
| # | Structure | K_{D} (μM) |
| A1 | 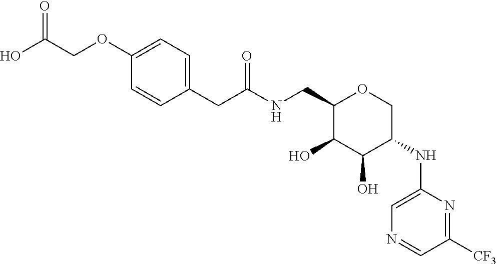 [SMILES: O=C(O)COc1ccc(CC(=O)NC[C@H]2OC[C@H](Nc3cncc(C(F)(F)F)n3)[C@@H](O)[C@H]2O)cc1] | +++ |
| A2 | 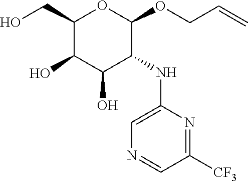 [SMILES: C=CCO[C@@H]1O[C@H](CO)[C@H](O)[C@H](O)[C@H]1Nc1cncc(C(F)(F)F)n1] | |
| A3 | 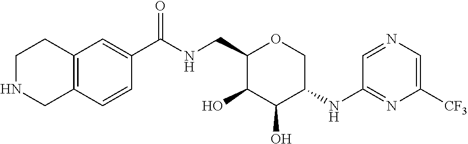 [SMILES: O=C(NC[C@H]1OC[C@H](Nc2cncc(C(F)(F)F)n2)[C@@H](O)[C@H]1O)c1ccc2c(c1)CCNC2] | +++ |
| A4 | 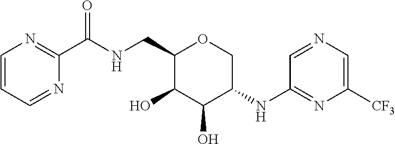 [SMILES: O=C(NC[C@H]1OC[C@H](Nc2cncc(C(F)(F)F)n2)[C@@H](O)[C@H]1O)c1ncccn1] | +++ |
| A5 | 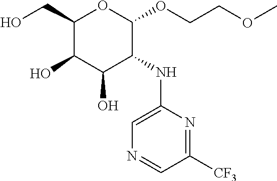 [SMILES: COCCO[C@H]1O[C@H](CO)[C@H](O)[C@H](O)[C@H]1Nc1cncc(C(F)(F)F)n1] | +++ |
| A6 | 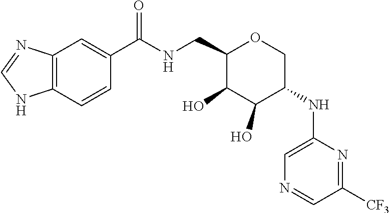 [SMILES: O=C(NC[C@H]1OC[C@H](Nc2cncc(C(F)(F)F)n2)[C@@H](O)[C@H]1O)c1ccc2[nH]cnc2c1] | +++ |
| A7 | 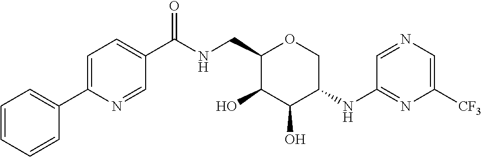 [SMILES: O=C(NC[C@H]1OC[C@H](Nc2cncc(C(F)(F)F)n2)[C@@H](O)[C@H]1O)c1ccc(-c2ccccc2)nc1] | +++ |
| A8 | 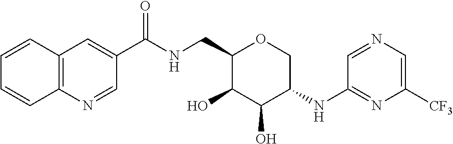 [SMILES: O=C(NC[C@H]1OC[C@H](Nc2cncc(C(F)(F)F)n2)[C@@H](O)[C@H]1O)c1cnc2ccccc2c1] | +++ |
| A9 | 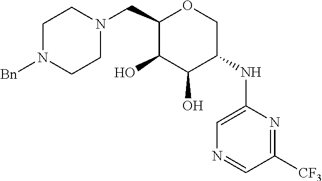 [SMILES: O[C@@H]1[C@H](O)[C@@H](Nc2cncc(C(F)(F)F)n2)CO[C@@H]1CN1CCN(Cc2ccccc2)CC1] | +++ |
| A10 | 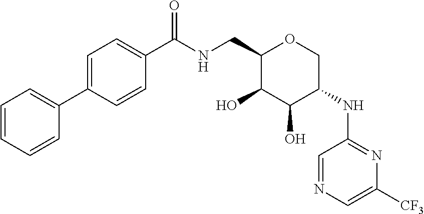 [SMILES: O=C(NC[C@H]1OC[C@H](Nc2cncc(C(F)(F)F)n2)[C@@H](O)[C@H]1O)c1ccc(-c2ccccc2)cc1] | +++ |
| A11 | 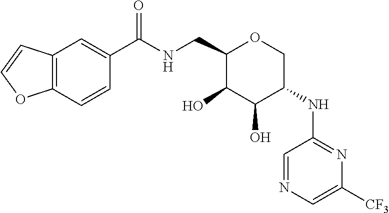 [SMILES: O=C(NC[C@H]1OC[C@H](Nc2cncc(C(F)(F)F)n2)[C@@H](O)[C@H]1O)c1ccc2occc2c1] | +++ |
| A12 | 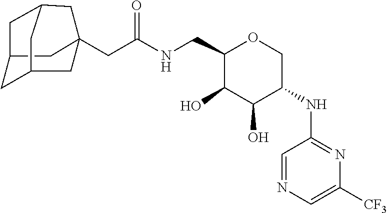 [SMILES: O=C(CC12CC3CC(CC(C3)C1)C2)NC[C@H]1OC[C@H](Nc2cncc(C(F)(F)F)n2)[C@@H](O)[C@H]1O] | +++ |
| A13 | 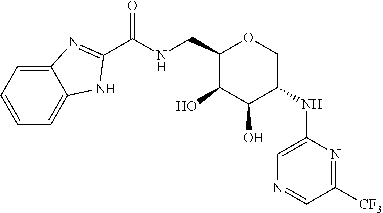 [SMILES: O=C(NC[C@H]1OC[C@H](Nc2cncc(C(F)(F)F)n2)[C@@H](O)[C@H]1O)c1nc2ccccc2[nH]1] | +++ |
| A14 | 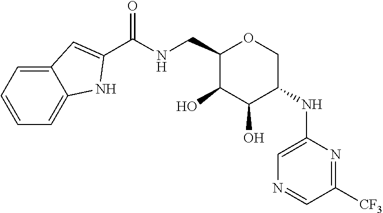 [SMILES: O=C(NC[C@H]1OC[C@H](Nc2cncc(C(F)(F)F)n2)[C@@H](O)[C@H]1O)c1cc2ccccc2[nH]1] | +++ |
| A15 | 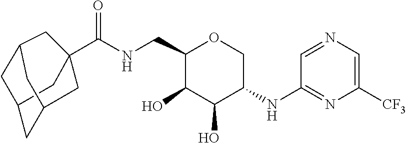 [SMILES: O=C(NC[C@H]1OC[C@H](Nc2cncc(C(F)(F)F)n2)[C@@H](O)[C@H]1O)C12CC3CC(CC(C3)C1)C2] | +++ |
| A16 | 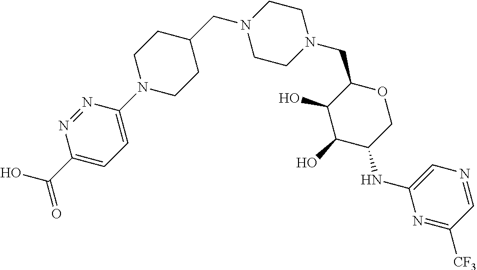 [SMILES: O=C(O)c1ccc(N2CCC(CN3CCN(C[C@H]4OC[C@H](Nc5cncc(C(F)(F)F)n5)[C@@H](O)[C@H]4O)CC3)CC2)nn1] | +++ |
| A17 | 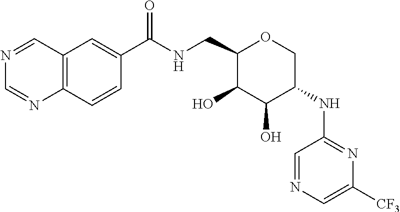 [SMILES: O=C(NC[C@H]1OC[C@H](Nc2cncc(C(F)(F)F)n2)[C@@H](O)[C@H]1O)c1ccc2ncncc2c1] | +++ |
| A18 | 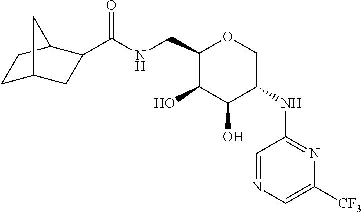 [SMILES: O=C(NC[C@H]1OC[C@H](Nc2cncc(C(F)(F)F)n2)[C@@H](O)[C@H]1O)C1CC2CCC1C2] | +++ |
| A19 | 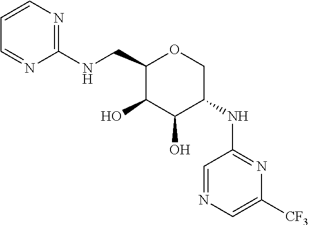 [SMILES: O[C@@H]1[C@H](O)[C@@H](Nc2cncc(C(F)(F)F)n2)CO[C@@H]1CNc1ncccn1] | +++ |
| A20 | 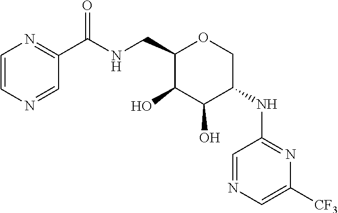 [SMILES: O=C(NC[C@H]1OC[C@H](Nc2cncc(C(F)(F)F)n2)[C@@H](O)[C@H]1O)c1cnccn1] | +++ |
| A21 | 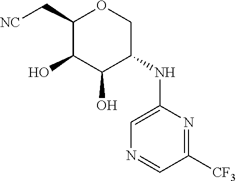 [SMILES: N#CC[C@H]1OC[C@H](Nc2cncc(C(F)(F)F)n2)[C@@H](O)[C@H]1O] | +++ |
| A22 | 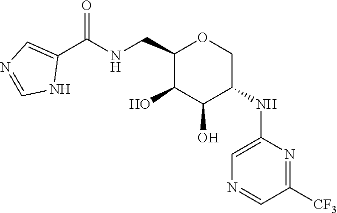 [SMILES: O=C(NC[C@H]1OC[C@H](Nc2cncc(C(F)(F)F)n2)[C@@H](O)[C@H]1O)c1cnc[nH]1] | +++ |
| A23 | 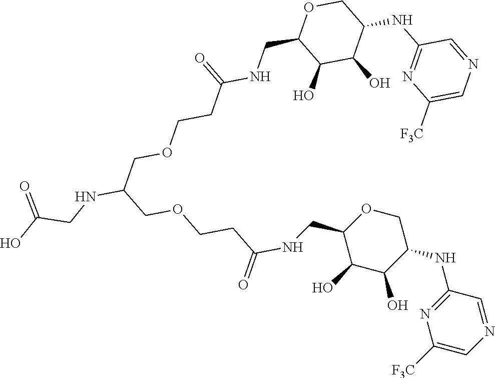 [SMILES: O=C(O)CNC(COCCC(=O)NC[C@H]1OC[C@H](Nc2cncc(C(F)(F)F)n2)[C@@H](O)[C@H]1O)COCCC(=O)NC[C@H]1OC[C@H](Nc2cncc(C(F)(F)F)n2)[C@@H](O)[C@H]1O] | >20 |
| A24 | 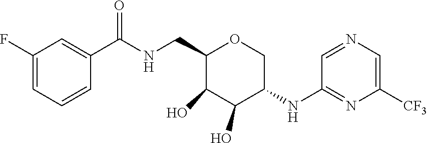 [SMILES: O=C(NC[C@H]1OC[C@H](Nc2cncc(C(F)(F)F)n2)[C@@H](O)[C@H]1O)c1cccc(F)c1] | +++ |
| A25 | 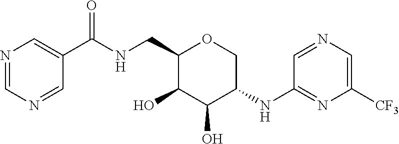 [SMILES: O=C(NC[C@H]1OC[C@H](Nc2cncc(C(F)(F)F)n2)[C@@H](O)[C@H]1O)c1cncnc1] | +++ |
| A26 | 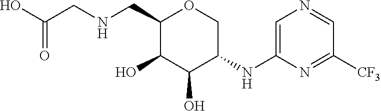 [SMILES: O=C(O)CNC[C@H]1OC[C@H](Nc2cncc(C(F)(F)F)n2)[C@@H](O)[C@H]1O] | +++ |
| A27 | 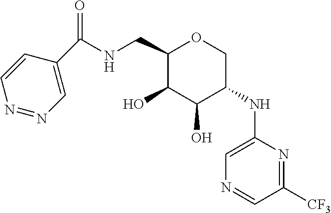 [SMILES: O=C(NC[C@H]1OC[C@H](Nc2cncc(C(F)(F)F)n2)[C@@H](O)[C@H]1O)c1ccnnc1] | +++ |
| A28 | 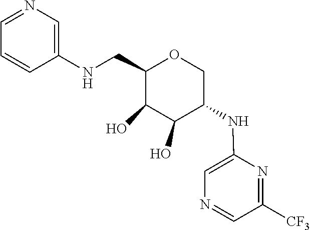 [SMILES: O[C@@H]1[C@H](O)[C@@H](Nc2cncc(C(F)(F)F)n2)CO[C@@H]1CNc1cccnc1] | +++ |
| A29 | 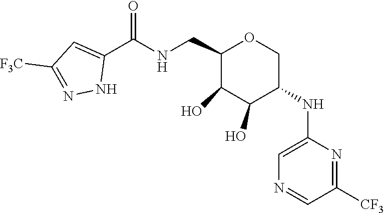 [SMILES: O=C(NC[C@H]1OC[C@H](Nc2cncc(C(F)(F)F)n2)[C@@H](O)[C@H]1O)c1cc(C(F)(F)F)n[nH]1] | +++ |
| A30 | 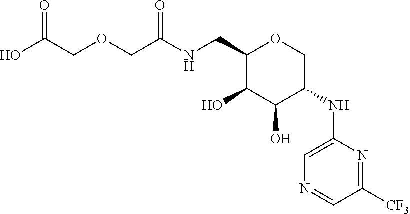 [SMILES: O=C(O)COCC(=O)NC[C@H]1OC[C@H](Nc2cncc(C(F)(F)F)n2)[C@@H](O)[C@H]1O] | +++ |
| A31 | 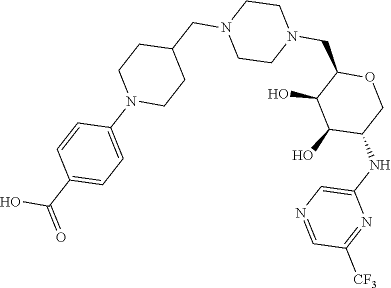 [SMILES: O=C(O)c1ccc(N2CCC(CN3CCN(C[C@H]4OC[C@H](Nc5cncc(C(F)(F)F)n5)[C@@H](O)[C@H]4O)CC3)CC2)cc1] | +++ |
| A32 | 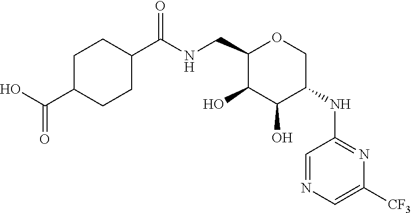 [SMILES: O=C(O)C1CCC(C(=O)NC[C@H]2OC[C@H](Nc3cncc(C(F)(F)F)n3)[C@@H](O)[C@H]2O)CC1] | +++ |
| A33 | 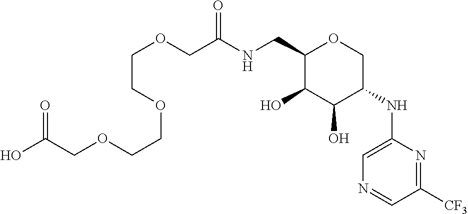 [SMILES: O=C(O)COCCOCCOCC(=O)NC[C@H]1OC[C@H](Nc2cncc(C(F)(F)F)n2)[C@@H](O)[C@H]1O] | +++ |
| A34 | 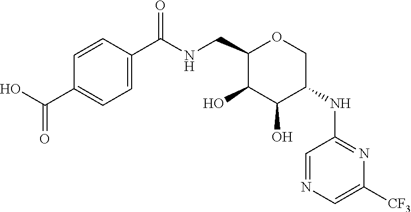 [SMILES: O=C(O)c1ccc(C(=O)NC[C@H]2OC[C@H](Nc3cncc(C(F)(F)F)n3)[C@@H](O)[C@H]2O)cc1] | +++ |
| A35 | 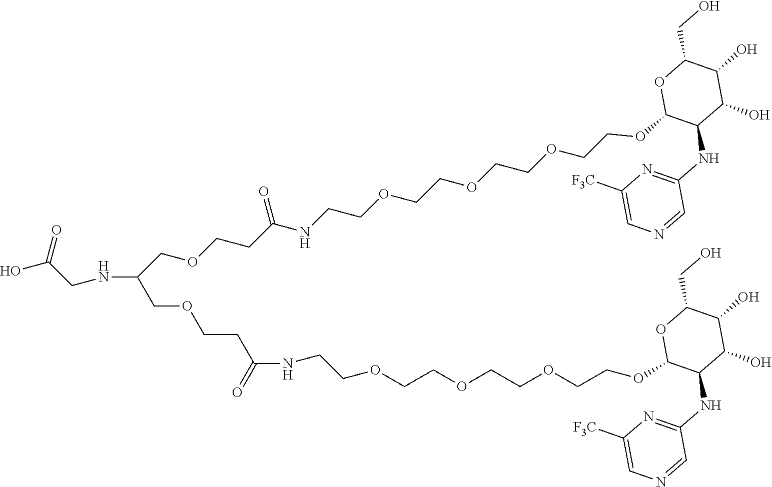 [SMILES: O=C(O)CNC(COCCC(=O)NCCOCCOCCOCCO[C@@H]1O[C@H](CO)[C@H](O)[C@H](O)[C@H]1Nc1cncc(C(F)(F)F)n1)COCCC(=O)NCCOCCOCCOCCO[C@@H]1O[C@H](CO)[C@H](O)[C@H](O)[C@H]1Nc1cncc(C(F)(F)F)n1] | +++ |
| A36 | 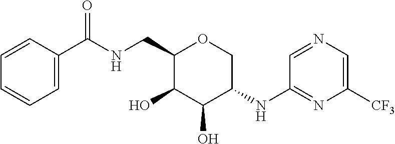 [SMILES: O=C(NC[C@H]1OC[C@H](Nc2cncc(C(F)(F)F)n2)[C@@H](O)[C@H]1O)c1ccccc1] | +++ |
| A37 | 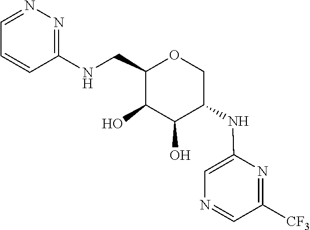 [SMILES: O[C@@H]1[C@H](O)[C@@H](Nc2cncc(C(F)(F)F)n2)CO[C@@H]1CNc1cccnn1] | +++ |
| A38 | 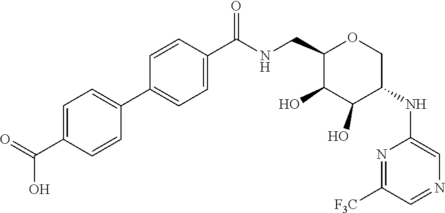 [SMILES: O=C(O)c1ccc(-c2ccc(C(=O)NC[C@H]3OC[C@H](Nc4cncc(C(F)(F)F)n4)[C@@H](O)[C@H]3O)cc2)cc1] | +++ |
| A39 | 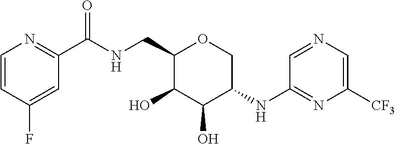 [SMILES: O=C(NC[C@H]1OC[C@H](Nc2cncc(C(F)(F)F)n2)[C@@H](O)[C@H]1O)c1cc(F)ccn1] | +++ |
| A40 | 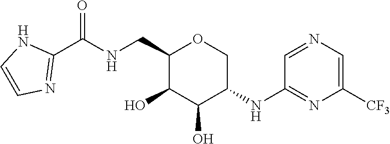 [SMILES: O=C(NC[C@H]1OC[C@H](Nc2cncc(C(F)(F)F)n2)[C@@H](O)[C@H]1O)c1ncc[nH]1] | +++ |
| A41 | 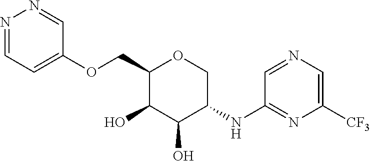 [SMILES: O[C@@H]1[C@H](O)[C@@H](Nc2cncc(C(F)(F)F)n2)CO[C@@H]1COc1ccnnc1] | +++ |
| A42 | 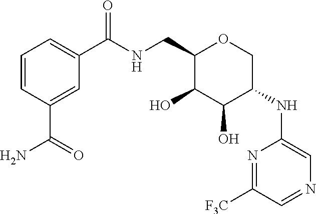 [SMILES: NC(=O)c1cccc(C(=O)NC[C@H]2OC[C@H](Nc3cncc(C(F)(F)F)n3)[C@@H](O)[C@H]2O)c1] | +++ |
| A43 | 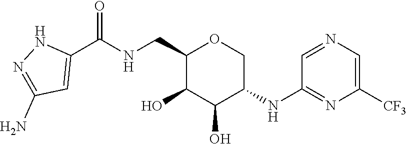 [SMILES: Nc1cc(C(=O)NC[C@H]2OC[C@H](Nc3cncc(C(F)(F)F)n3)[C@@H](O)[C@H]2O)[nH]n1] | +++ |
| A44 | 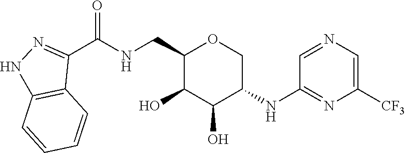 [SMILES: O=C(NC[C@H]1OC[C@H](Nc2cncc(C(F)(F)F)n2)[C@@H](O)[C@H]1O)c1n[nH]c2ccccc12] | +++ |
| A45 | 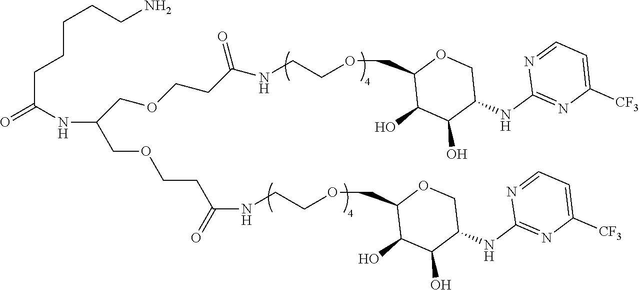 | +++ |
| A46 | 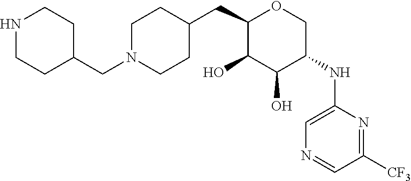 [SMILES: O[C@@H]1[C@H](O)[C@@H](Nc2cncc(C(F)(F)F)n2)CO[C@@H]1CC1CCN(CC2CCNCC2)CC1] | +++ |
| A47 | 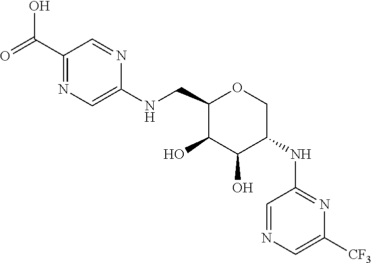 [SMILES: O=C(O)c1cnc(NC[C@H]2OC[C@H](Nc3cncc(C(F)(F)F)n3)[C@@H](O)[C@H]2O)cn1] | |
| A48 | 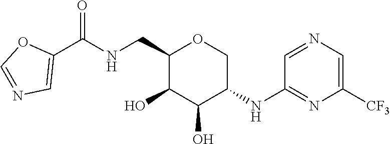 [SMILES: O=C(NC[C@H]1OC[C@H](Nc2cncc(C(F)(F)F)n2)[C@@H](O)[C@H]1O)c1cnco1] | +++ |
| A49 | 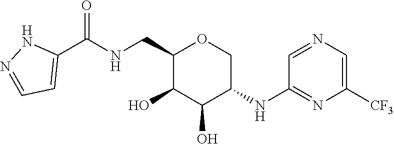 [SMILES: O=C(NC[C@H]1OC[C@H](Nc2cncc(C(F)(F)F)n2)[C@@H](O)[C@H]1O)c1ccn[nH]1] | +++ |
| A50 | 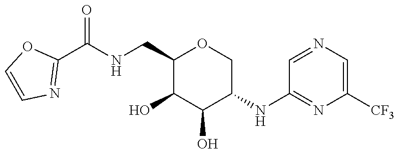 [SMILES: O=C(NC[C@H]1OC[C@H](Nc2cncc(C(F)(F)F)n2)[C@@H](O)[C@H]1O)c1ncco1] | +++ |
| A52 | 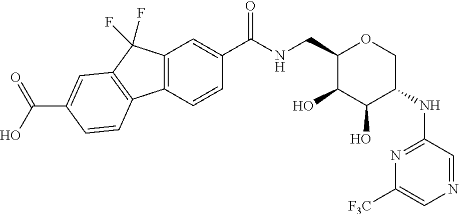 [SMILES: O=C(O)c1ccc2c(c1)C(F)(F)c1cc(C(=O)NC[C@H]3OC[C@H](Nc4cncc(C(F)(F)F)n4)[C@@H](O)[C@H]3O)ccc1-2] | +++ |
| A53 | 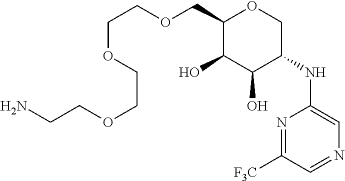 [SMILES: NCCOCCOCCOC[C@H]1OC[C@H](Nc2cncc(C(F)(F)F)n2)[C@@H](O)[C@H]1O] | +++ |
| A54 | 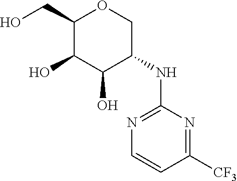 [SMILES: OC[C@H]1OC[C@H](Nc2nccc(C(F)(F)F)n2)[C@@H](O)[C@H]1O] | ++ |
| A55 | 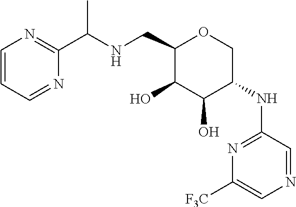 [SMILES: CC(NC[C@H]1OC[C@H](Nc2cncc(C(F)(F)F)n2)[C@@H](O)[C@H]1O)c1ncccn1] | +++ |
| A56 | 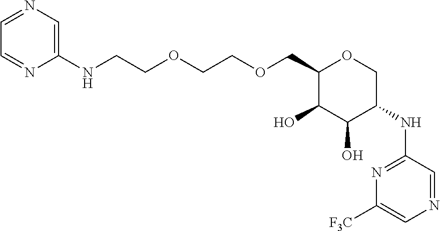 [SMILES: O[C@@H]1[C@H](O)[C@@H](Nc2cncc(C(F)(F)F)n2)CO[C@@H]1COCCOCCNc1cnccn1] | +++ |
| A57 | 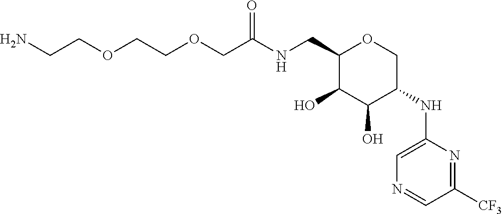 [SMILES: NCCOCCOCC(=O)NC[C@H]1OC[C@H](Nc2cncc(C(F)(F)F)n2)[C@@H](O)[C@H]1O] | +++ |
| A58 | 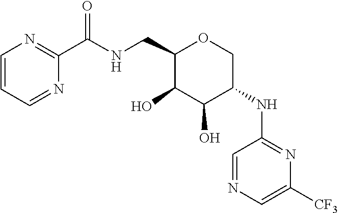 [SMILES: O=C(NC[C@H]1OC[C@H](Nc2cncc(C(F)(F)F)n2)[C@@H](O)[C@H]1O)c1ncccn1] | +++ |
| A59 | 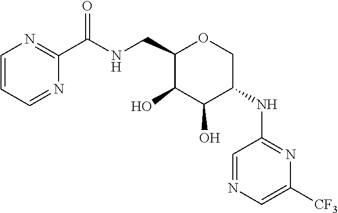 [SMILES: O=C(NC[C@H]1OC[C@H](Nc2cncc(C(F)(F)F)n2)[C@@H](O)[C@H]1O)c1ncccn1] | +++ |
| A60 | 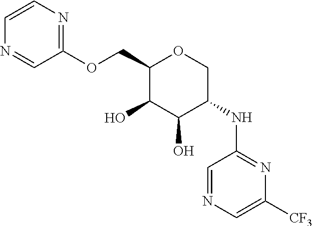 [SMILES: O[C@@H]1[C@H](O)[C@@H](Nc2cncc(C(F)(F)F)n2)CO[C@@H]1COc1cnccn1] | +++ |
| A61 | 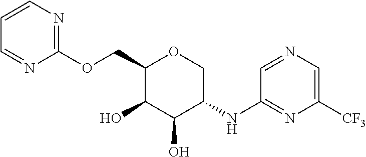 [SMILES: O[C@@H]1[C@H](O)[C@@H](Nc2cncc(C(F)(F)F)n2)CO[C@@H]1COc1ncccn1] | +++ |
| A62 | 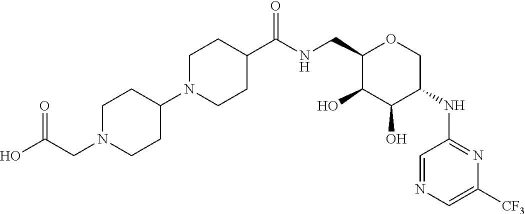 [SMILES: O=C(O)CN1CCC(N2CCC(C(=O)NC[C@H]3OC[C@H](Nc4cncc(C(F)(F)F)n4)[C@@H](O)[C@H]3O)CC2)CC1] | +++ |
| A63 | 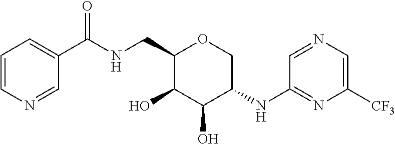 [SMILES: O=C(NC[C@H]1OC[C@H](Nc2cncc(C(F)(F)F)n2)[C@@H](O)[C@H]1O)c1cccnc1] | +++ |
| A64 | 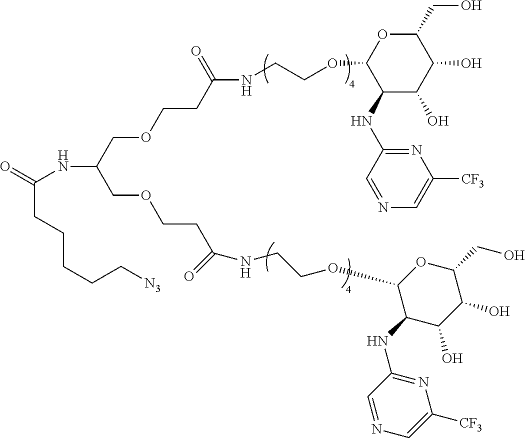 | +++ |
| A65 | 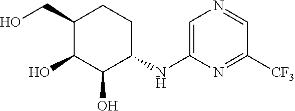 [SMILES: OC[C@H]1CC[C@H](Nc2cncc(C(F)(F)F)n2)[C@@H](O)[C@H]1O] | +++ |
| A66 | 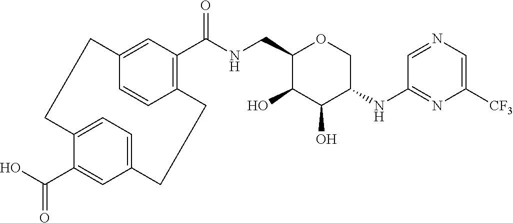 [SMILES: O=C(O)c1cc2ccc1CCc1ccc(c(C(=O)NC[C@H]3OC[C@H](Nc4cncc(C(F)(F)F)n4)[C@@H](O)[C@H]3O)c1)CC2] | +++ |
| A67 | 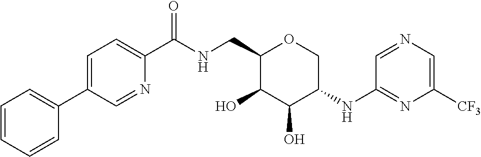 [SMILES: O=C(NC[C@H]1OC[C@H](Nc2cncc(C(F)(F)F)n2)[C@@H](O)[C@H]1O)c1ccc(-c2ccccc2)cn1] | +++ |
| A68 | 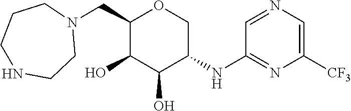 [SMILES: O[C@@H]1[C@H](O)[C@@H](Nc2cncc(C(F)(F)F)n2)CO[C@@H]1CN1CCCNCC1] | +++ |
| A69 | 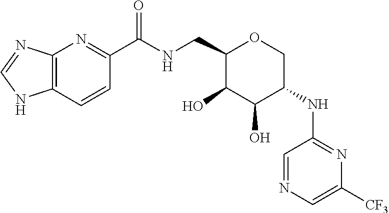 [SMILES: O=C(NC[C@H]1OC[C@H](Nc2cncc(C(F)(F)F)n2)[C@@H](O)[C@H]1O)c1ccc2[nH]cnc2n1] | +++ |
| A70 | 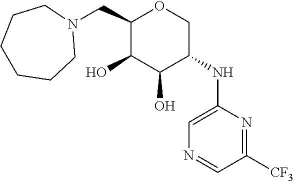 [SMILES: O[C@@H]1[C@H](O)[C@@H](Nc2cncc(C(F)(F)F)n2)CO[C@@H]1CN1CCCCCC1] | +++ |
| A71 | 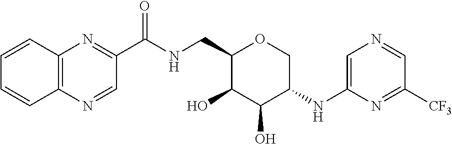 [SMILES: O=C(NC[C@H]1OC[C@H](Nc2cncc(C(F)(F)F)n2)[C@@H](O)[C@H]1O)c1cnc2ccccc2n1] | +++ |
| A72 | 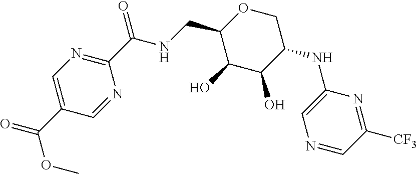 [SMILES: COC(=O)c1cnc(C(=O)NC[C@H]2OC[C@H](Nc3cncc(C(F)(F)F)n3)[C@@H](O)[C@H]2O)nc1] | +++ |
| A73 | 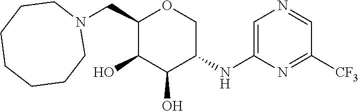 [SMILES: O[C@@H]1[C@H](O)[C@@H](Nc2cncc(C(F)(F)F)n2)CO[C@@H]1CN1CCCCCCC1] | +++ |
| A74 | 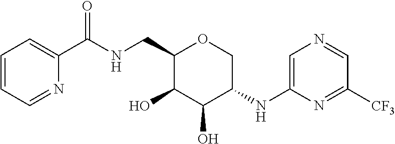 [SMILES: O=C(NC[C@H]1OC[C@H](Nc2cncc(C(F)(F)F)n2)[C@@H](O)[C@H]1O)c1ccccn1] | +++ |
| A75 | 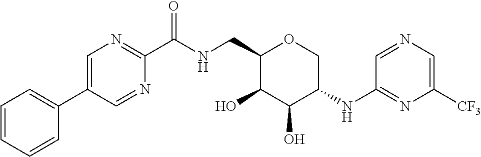 [SMILES: O=C(NC[C@H]1OC[C@H](Nc2cncc(C(F)(F)F)n2)[C@@H](O)[C@H]1O)c1ncc(-c2ccccc2)cn1] | +++ |
| A76 | 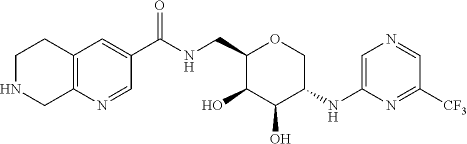 [SMILES: O=C(NC[C@H]1OC[C@H](Nc2cncc(C(F)(F)F)n2)[C@@H](O)[C@H]1O)c1cnc2c(c1)CCNC2] | +++ |
| A77 | 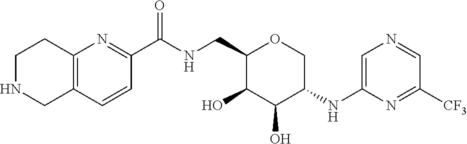 [SMILES: O=C(NC[C@H]1OC[C@H](Nc2cncc(C(F)(F)F)n2)[C@@H](O)[C@H]1O)c1ccc2c(n1)CCNC2] | +++ |
| A78 | 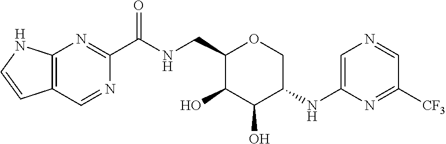 [SMILES: O=C(NC[C@H]1OC[C@H](Nc2cncc(C(F)(F)F)n2)[C@@H](O)[C@H]1O)c1ncc2cc[nH]c2n1] | +++ |
| A79 | 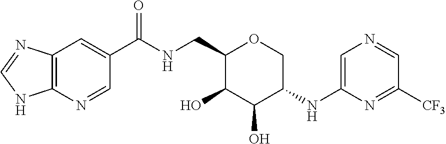 [SMILES: O=C(NC[C@H]1OC[C@H](Nc2cncc(C(F)(F)F)n2)[C@@H](O)[C@H]1O)c1cnc2[nH]cnc2c1] | +++ |
| A80 | 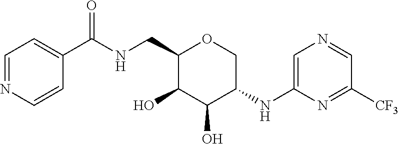 [SMILES: O=C(NC[C@H]1OC[C@H](Nc2cncc(C(F)(F)F)n2)[C@@H](O)[C@H]1O)c1ccncc1] | +++ |
| A81 | 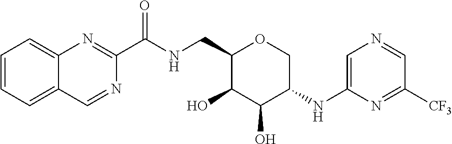 [SMILES: O=C(NC[C@H]1OC[C@H](Nc2cncc(C(F)(F)F)n2)[C@@H](O)[C@H]1O)c1ncc2ccccc2n1] | +++ |
| A82 | 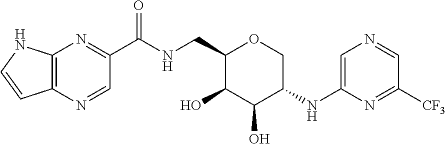 [SMILES: O=C(NC[C@H]1OC[C@H](Nc2cncc(C(F)(F)F)n2)[C@@H](O)[C@H]1O)c1cnc2cc[nH]c2n1] | +++ |
| A83 | 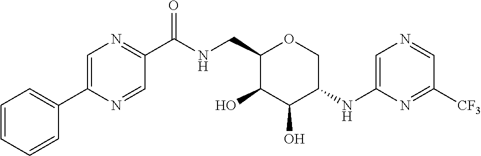 [SMILES: O=C(NC[C@H]1OC[C@H](Nc2cncc(C(F)(F)F)n2)[C@@H](O)[C@H]1O)c1cnc(-c2ccccc2)cn1] | +++ |
| A84 | 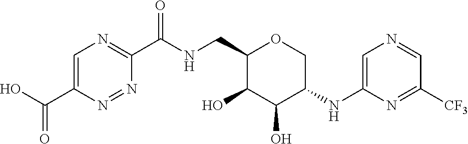 [SMILES: O=C(O)c1cnc(C(=O)NC[C@H]2OC[C@H](Nc3cncc(C(F)(F)F)n3)[C@@H](O)[C@H]2O)nn1] | +++ |
| A85 | 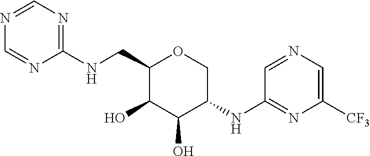 [SMILES: O[C@@H]1[C@H](O)[C@@H](Nc2cncc(C(F)(F)F)n2)CO[C@@H]1CNc1ncncn1] | +++ |
| A86 | 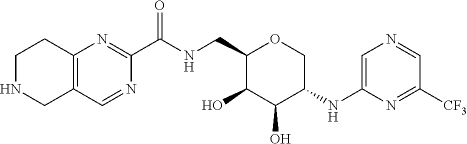 [SMILES: O=C(NC[C@H]1OC[C@H](Nc2cncc(C(F)(F)F)n2)[C@@H](O)[C@H]1O)c1ncc2c(n1)CCNC2] | +++ |
| A87 | 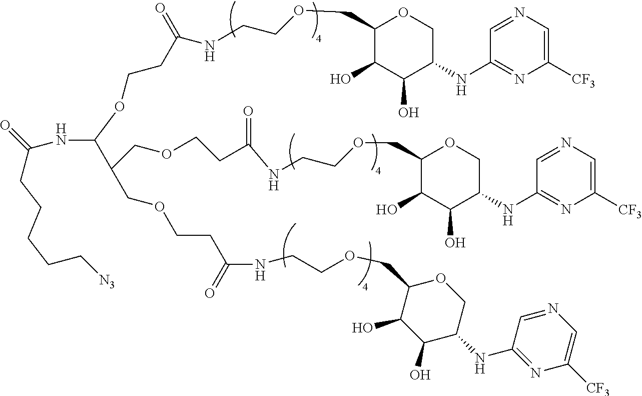 | +++ |
| A88 | 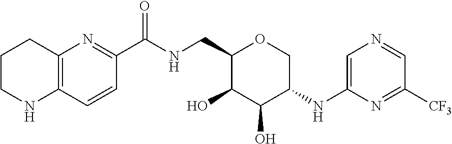 [SMILES: O=C(NC[C@H]1OC[C@H](Nc2cncc(C(F)(F)F)n2)[C@@H](O)[C@H]1O)c1ccc2c(n1)CCCN2] | +++ |
| A89 | 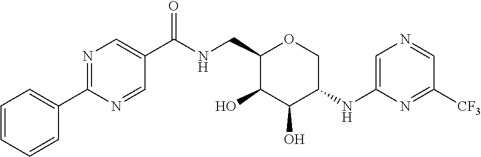 [SMILES: O=C(NC[C@H]1OC[C@H](Nc2cncc(C(F)(F)F)n2)[C@@H](O)[C@H]1O)c1cnc(-c2ccccc2)nc1] | +++ |
| A90 | 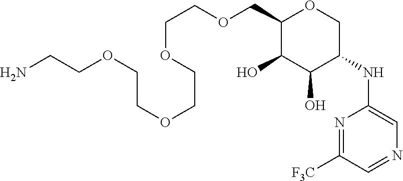 [SMILES: NCCOCCOCCOCCOC[C@H]1OC[C@H](Nc2cncc(C(F)(F)F)n2)[C@@H](O)[C@H]1O] | |
| A91 | 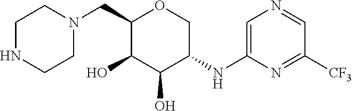 [SMILES: O[C@@H]1[C@H](O)[C@@H](Nc2cncc(C(F)(F)F)n2)CO[C@@H]1CN1CCNCC1] | +++ |
| A92 | 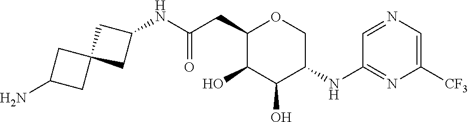 | +++ |
| A93 | 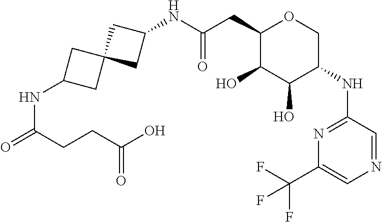 | +++ |
| A94 | 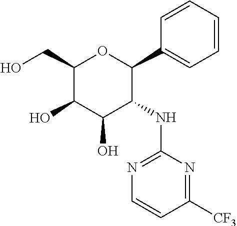 [SMILES: OC[C@H]1O[C@@H](c2ccccc2)[C@H](Nc2nccc(C(F)(F)F)n2)[C@@H](O)[C@H]1O] | +++ |
| A95 | 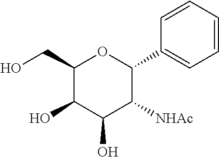 [SMILES: CC(=O)N[C@@H]1[C@@H](O)[C@@H](O)[C@@H](CO)O[C@@H]1c1ccccc1] | +++ |
| A96 | 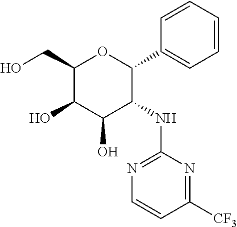 [SMILES: OC[C@H]1O[C@H](c2ccccc2)[C@H](Nc2nccc(C(F)(F)F)n2)[C@@H](O)[C@H]1O] | +++ |
| A97 | 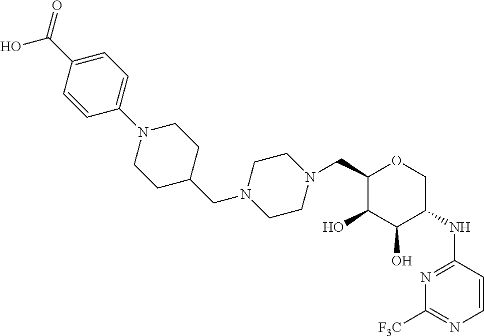 [SMILES: O=C(O)c1ccc(N2CCC(CN3CCN(C[C@H]4OC[C@H](Nc5ccnc(C(F)(F)F)n5)[C@@H](O)[C@H]4O)CC3)CC2)cc1] | +++ |
| A98 | 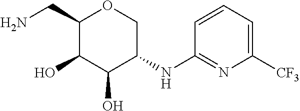 [SMILES: NC[C@H]1OC[C@H](Nc2cccc(C(F)(F)F)n2)[C@@H](O)[C@H]1O] | +++ |
| A99 | 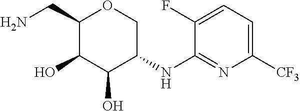 [SMILES: NC[C@H]1OC[C@H](Nc2nc(C(F)(F)F)ccc2F)[C@@H](O)[C@H]1O] | +++ |
| A100 | 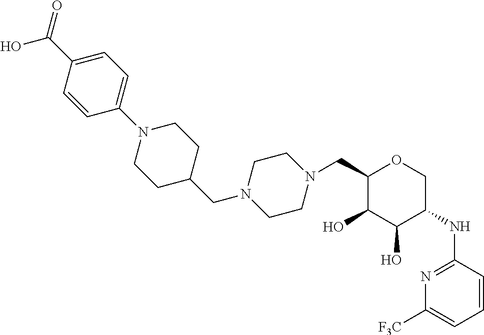 [SMILES: O=C(O)c1ccc(N2CCC(CN3CCN(C[C@H]4OC[C@H](Nc5cccc(C(F)(F)F)n5)[C@@H](O)[C@H]4O)CC3)CC2)cc1] | +++ |
| A101 | 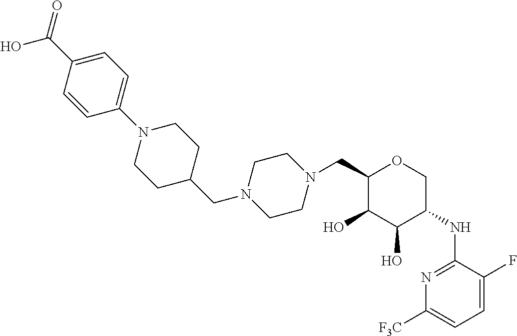 [SMILES: O=C(O)c1ccc(N2CCC(CN3CCN(C[C@H]4OC[C@H](Nc5nc(C(F)(F)F)ccc5F)[C@@H](O)[C@H]4O)CC3)CC2)cc1] | +++ |
| A102 | 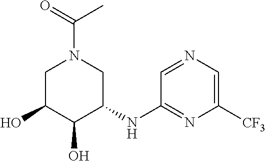 [SMILES: CC(=O)N1C[C@H](Nc2cncc(C(F)(F)F)n2)[C@@H](O)[C@@H](O)C1] | +++ |
| A103 | 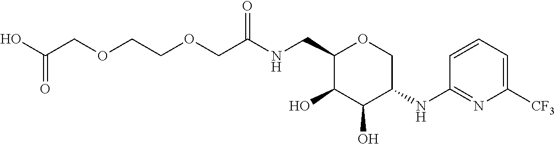 [SMILES: O=C(O)COCCOCC(=O)NC[C@H]1OC[C@H](Nc2cccc(C(F)(F)F)n2)[C@@H](O)[C@H]1O] | +++ |
| A104 | 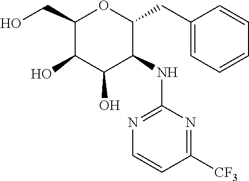 [SMILES: OC[C@H]1O[C@H](Cc2ccccc2)[C@@H](Nc2nccc(C(F)(F)F)n2)[C@@H](O)[C@H]1O] | ++ |
| A105 | 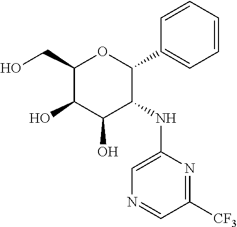 [SMILES: OC[C@H]1O[C@H](c2ccccc2)[C@H](Nc2cncc(C(F)(F)F)n2)[C@@H](O)[C@H]1O] | +++ |
| A106 | 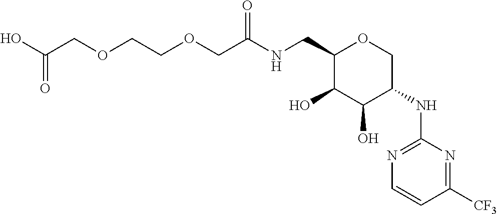 [SMILES: O=C(O)COCCOCC(=O)NC[C@H]1OC[C@H](Nc2nccc(C(F)(F)F)n2)[C@@H](O)[C@H]1O] | +++ |
| A107 | 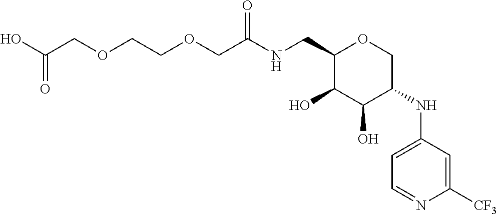 [SMILES: O=C(O)COCCOCC(=O)NC[C@H]1OC[C@H](Nc2ccnc(C(F)(F)F)c2)[C@@H](O)[C@H]1O] | +++ |
| A108 | 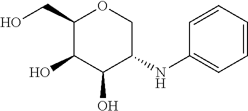 [SMILES: OC[C@H]1OC[C@H](Nc2ccccc2)[C@@H](O)[C@H]1O] | +++ |
| A109 | 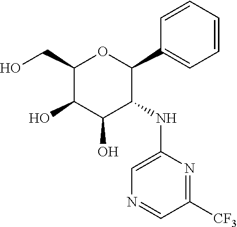 [SMILES: OC[C@H]1O[C@@H](c2ccccc2)[C@H](Nc2cncc(C(F)(F)F)n2)[C@@H](O)[C@H]1O] | ++ |
| A110 | 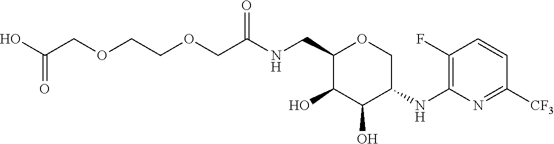 [SMILES: O=C(O)COCCOCC(=O)NC[C@H]1OC[C@H](Nc2nc(C(F)(F)F)ccc2F)[C@@H](O)[C@H]1O] | +++ |
| A111 | 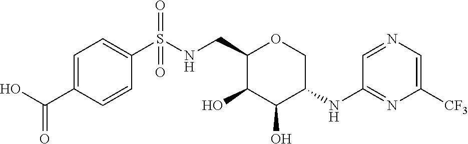 [SMILES: O=C(O)c1ccc(S(=O)(=O)NC[C@H]2OC[C@H](Nc3cncc(C(F)(F)F)n3)[C@@H](O)[C@H]2O)cc1] | +++ |
| A112 | 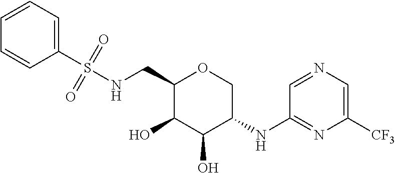 [SMILES: O=S(=O)(NC[C@H]1OC[C@H](Nc2cncc(C(F)(F)F)n2)[C@@H](O)[C@H]1O)c1ccccc1] | +++ |
| A113 | 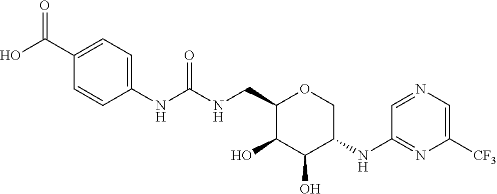 [SMILES: O=C(NC[C@H]1OC[C@H](Nc2cncc(C(F)(F)F)n2)[C@@H](O)[C@H]1O)Nc1ccc(C(=O)O)cc1] | +++ |
| A114 | 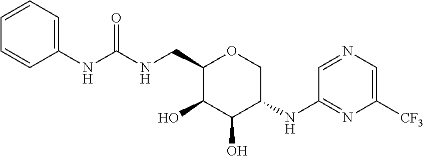 [SMILES: O=C(NC[C@H]1OC[C@H](Nc2cncc(C(F)(F)F)n2)[C@@H](O)[C@H]1O)Nc1ccccc1] | +++ |
| A115 | 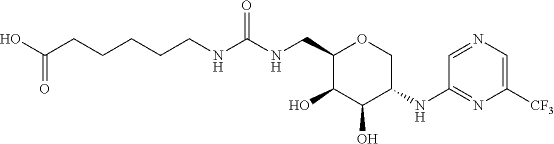 [SMILES: O=C(O)CCCCCNC(=O)NC[C@H]1OC[C@H](Nc2cncc(C(F)(F)F)n2)[C@@H](O)[C@H]1O] | +++ |
| A116 | 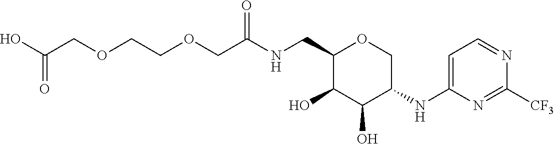 [SMILES: O=C(O)COCCOCC(=O)NC[C@H]1OC[C@H](Nc2ccnc(C(F)(F)F)n2)[C@@H](O)[C@H]1O] | +++ |
| A117 | 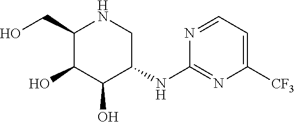 [SMILES: OC[C@H]1NC[C@H](Nc2nccc(C(F)(F)F)n2)[C@@H](O)[C@H]1O] | +++ |
| A118 | 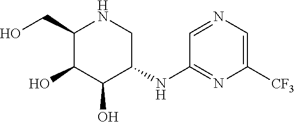 [SMILES: OC[C@H]1NC[C@H](Nc2cncc(C(F)(F)F)n2)[C@@H](O)[C@H]1O] | +++ |
| A119 | 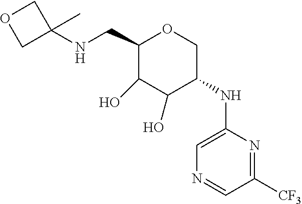 [SMILES: CC1(NC[C@H]2OC[C@H](Nc3cncc(C(F)(F)F)n3)C(O)C2O)COC1] | |
| A120 | 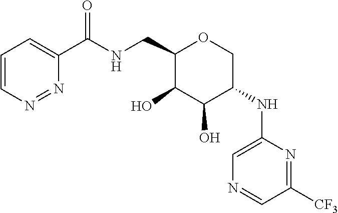 [SMILES: O=C(NC[C@H]1OC[C@H](Nc2cncc(C(F)(F)F)n2)[C@@H](O)[C@H]1O)c1cccnn1] | |
| A121 | 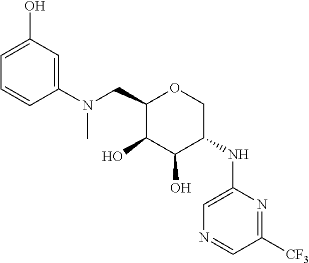 [SMILES: CN(C[C@H]1OC[C@H](Nc2cncc(C(F)(F)F)n2)[C@@H](O)[C@H]1O)c1cccc(O)c1] | |
| A122 | 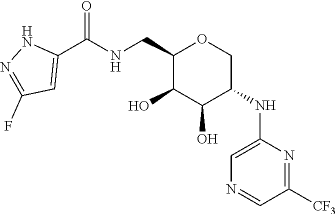 [SMILES: O=C(NC[C@H]1OC[C@H](Nc2cncc(C(F)(F)F)n2)[C@@H](O)[C@H]1O)c1cc(F)n[nH]1] | |
| A123 | 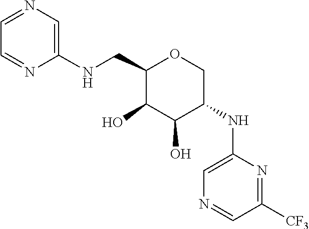 [SMILES: O[C@@H]1[C@H](O)[C@@H](Nc2cncc(C(F)(F)F)n2)CO[C@@H]1CNc1cnccn1] | |
| A124 | 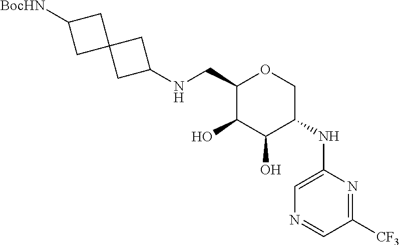 [SMILES: CC(C)(C)OC(=O)NC1CC2(CC(NC[C@H]3OC[C@H](Nc4cncc(C(F)(F)F)n4)[C@@H](O)[C@H]3O)C2)C1] | |
| A125 | 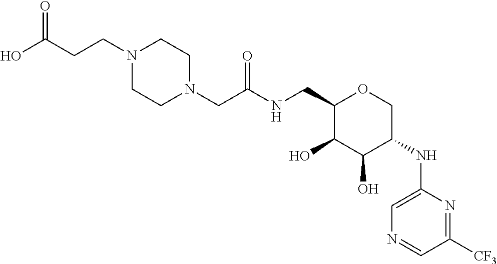 [SMILES: O=C(O)CCN1CCN(CC(=O)NC[C@H]2OC[C@H](Nc3cncc(C(F)(F)F)n3)[C@@H](O)[C@H]2O)CC1] | |
| A126 | 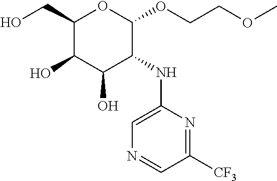 [SMILES: COCCO[C@H]1O[C@H](CO)[C@H](O)[C@H](O)[C@H]1Nc1cncc(C(F)(F)F)n1] | |
| A127 | 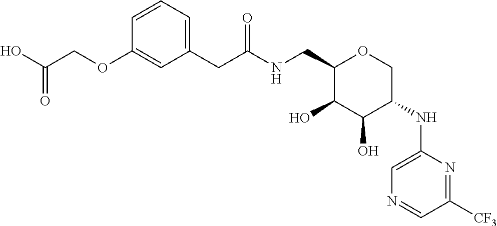 [SMILES: O=C(O)COc1cccc(CC(=O)NC[C@H]2OC[C@H](Nc3cncc(C(F)(F)F)n3)[C@@H](O)[C@H]2O)c1] | |
| A128 | 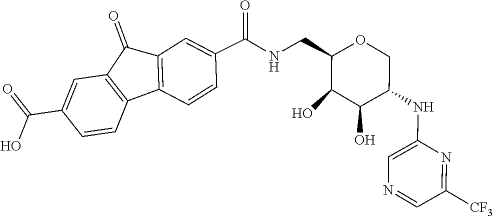 [SMILES: O=C(O)c1ccc2c(c1)C(=O)c1cc(C(=O)NC[C@H]3OC[C@H](Nc4cncc(C(F)(F)F)n4)[C@@H](O)[C@H]3O)ccc1-2] | |
| A129 | 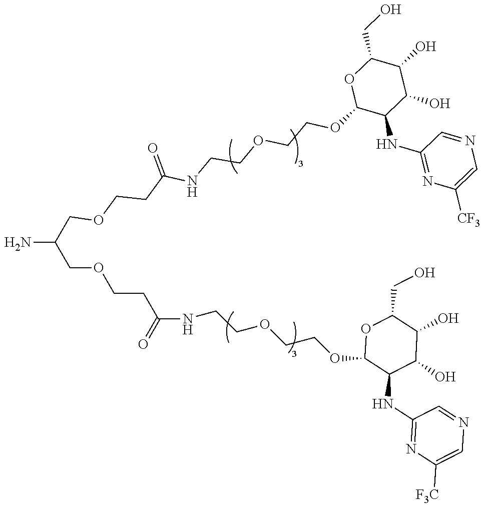 | |
| A130 | 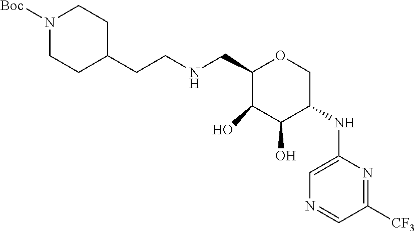 [SMILES: CC(C)(C)OC(=O)N1CCC(CCNC[C@H]2OC[C@H](Nc3cncc(C(F)(F)F)n3)[C@@H](O)[C@H]2O)CC1] | |
| A131 | 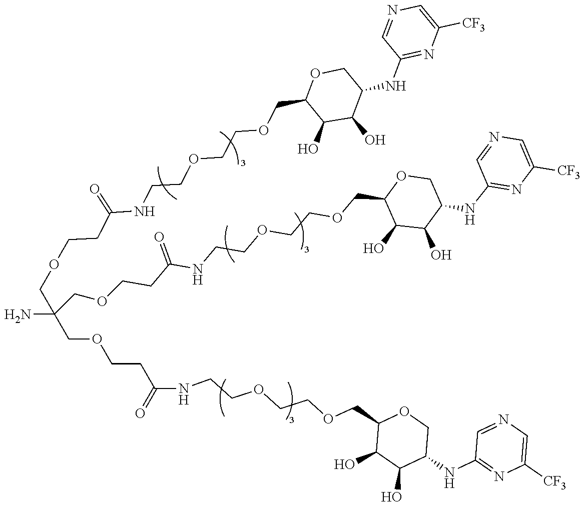 | |
| A132 | 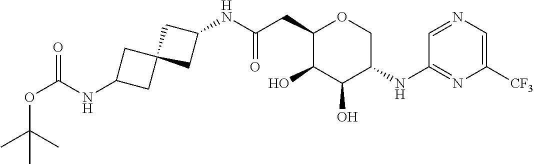 | |
| A133 | 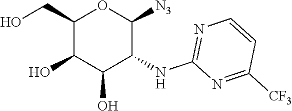 [SMILES: [N-]=[N+]=N[C@@H]1O[C@H](CO)[C@H](O)[C@H](O)[C@H]1Nc1nccc(C(F)(F)F)n1] | |
| A134 | 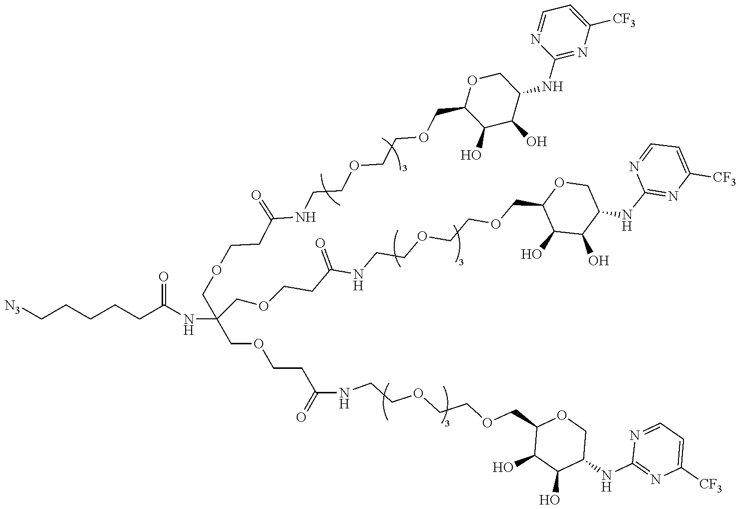 | |
| A135 | 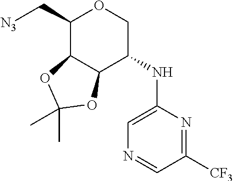 [SMILES: CC1(C)O[C@@H]2[C@H](O1)[C@@H](Nc1cncc(C(F)(F)F)n1)CO[C@@H]2CN=[N+]=[N-]] | |
| A136 | 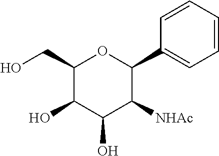 [SMILES: CC(=O)N[C@H]1[C@@H](O)[C@@H](O)[C@@H](CO)O[C@H]1c1ccccc1] | |
| A137 | 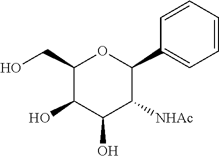 [SMILES: CC(=O)N[C@@H]1[C@@H](O)[C@@H](O)[C@@H](CO)O[C@H]1c1ccccc1] | |
| A138 | 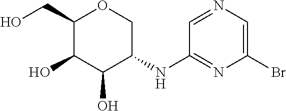 [SMILES: OC[C@H]1OC[C@H](Nc2cncc(Br)n2)[C@@H](O)[C@H]1O] | +++ |

In the table above, K_{d} values that are >=100 μM=+, <100 μM and >10 μM=++ and <10 μM=+++

TABLE 1B

| | | ASGPR Kd |
|---|---|---|
| Compound # | Structure | (μM) |
| A139 | 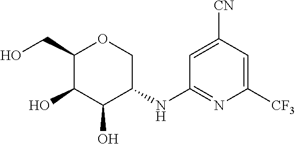 [SMILES: N#Cc1cc(N[C@H]2CO[C@H](CO)[C@H](O)[C@@H]2O)nc(C(F)(F)F)c1] | +++ |
| A140 | 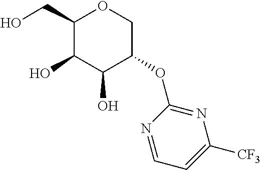 [SMILES: OC[C@H]1OC[C@H](Oc2nccc(C(F)(F)F)n2)[C@@H](O)[C@H]1O] | +++ |
| A141 | 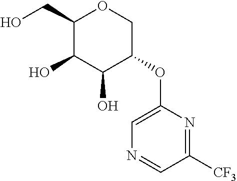 [SMILES: OC[C@H]1OC[C@H](Oc2cncc(C(F)(F)F)n2)[C@@H](O)[C@H]1O] | +++ |
| A142 | 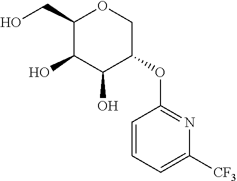 [SMILES: OC[C@H]1OC[C@H](Oc2cccc(C(F)(F)F)n2)[C@@H](O)[C@H]1O] | +++ |
| A143 | 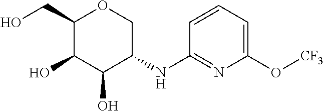 | +++ |
| A144 | 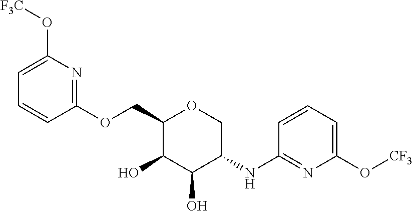 [SMILES: O[C@@H]1[C@H](O)[C@@H](Nc2cccc(OC(F)(F)F)n2)CO[C@@H]1COc1cccc(OC(F)(F)F)n1] | +++ |
| A145 | 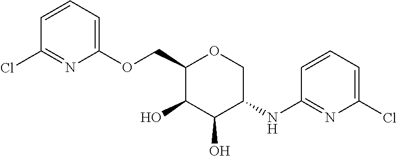 [SMILES: O[C@@H]1[C@H](O)[C@@H](Nc2cccc(Cl)n2)CO[C@@H]1COc1cccc(Cl)n1] | ++ |
| A146 | 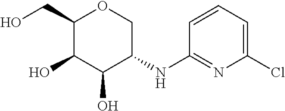 [SMILES: OC[C@H]1OC[C@H](Nc2cccc(Cl)n2)[C@@H](O)[C@H]1O] | +++ |
| A147 | 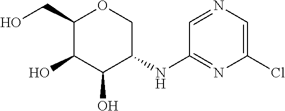 [SMILES: OC[C@H]1OC[C@H](Nc2cncc(Cl)n2)[C@@H](O)[C@H]1O] | +++ |
| A148 | 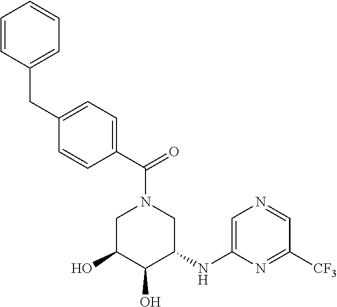 [SMILES: O=C(c1ccc(Cc2ccccc2)cc1)N1C[C@H](Nc2cncc(C(F)(F)F)n2)[C@@H](O)[C@@H](O)C1] | +++ |
| A149 | 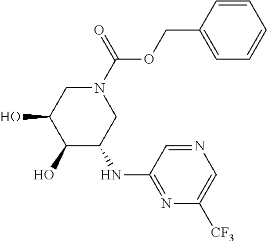 [SMILES: O=C(OCc1ccccc1)N1C[C@H](Nc2cncc(C(F)(F)F)n2)[C@@H](O)[C@@H](O)C1] | +++ |
| A150 | 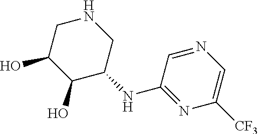 [SMILES: O[C@@H]1[C@@H](Nc2cncc(C(F)(F)F)n2)CNC[C@@H]1O] | +++ |
| A151 | 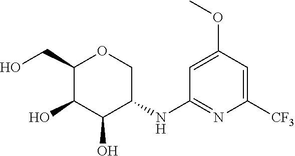 [SMILES: COc1cc(N[C@H]2CO[C@H](CO)[C@H](O)[C@@H]2O)nc(C(F)(F)F)c1] | +++ |
| A152 | 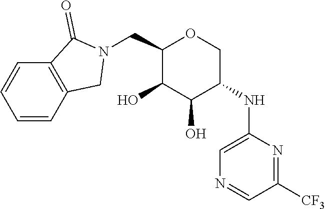 [SMILES: O=C1c2ccccc2CN1C[C@H]1OC[C@H](Nc2cncc(C(F)(F)F)n2)[C@@H](O)[C@H]1O] | +++ |
| A153 | 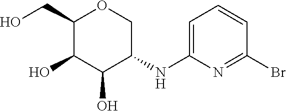 [SMILES: OC[C@H]1OC[C@H](Nc2cccc(Br)n2)[C@@H](O)[C@H]1O] | +++ |
| A154 | 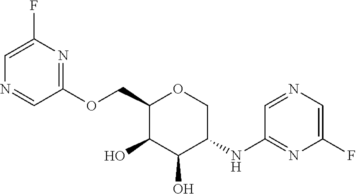 [SMILES: O[C@@H]1[C@H](O)[C@@H](Nc2cncc(F)n2)CO[C@@H]1COc1cncc(F)n1] | +++ |
| A155 | 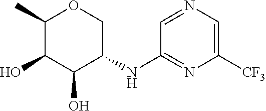 [SMILES: C[C@H]1OC[C@H](Nc2cncc(C(F)(F)F)n2)[C@@H](O)[C@H]1O] | +++ |
| A156 | 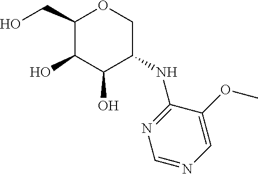 [SMILES: COc1cncnc1N[C@H]1CO[C@H](CO)[C@H](O)[C@@H]1O] | ++ |
| A157 | 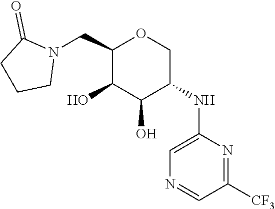 [SMILES: O=C1CCCN1C[C@H]1OC[C@H](Nc2cncc(C(F)(F)F)n2)[C@@H](O)[C@H]1O] | +++ |
| A158 | 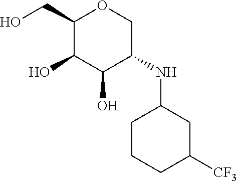 [SMILES: OC[C@H]1OC[C@H](NC2CCCC(C(F)(F)F)C2)[C@@H](O)[C@H]1O] | >30 |
| A159 | 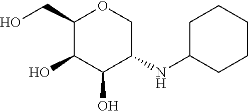 [SMILES: OC[C@H]1OC[C@H](NC2CCCCC2)[C@@H](O)[C@H]1O] | >30 |
| A160 | 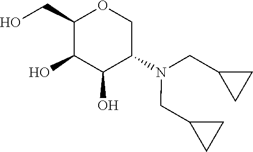 [SMILES: OC[C@H]1OC[C@H](N(CC2CC2)CC2CC2)[C@@H](O)[C@H]1O] | >30 |
| A161 | 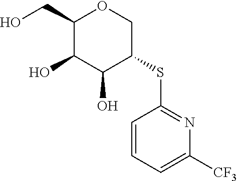 [SMILES: OC[C@H]1OC[C@H](Sc2cccc(C(F)(F)F)n2)[C@@H](O)[C@H]1O] | +++ |
| A162 | 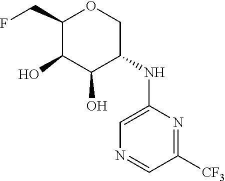 [SMILES: O[C@@H]1[C@H](O)[C@@H](Nc2cncc(C(F)(F)F)n2)CO[C@@H]1CF] | +++ |
| A163 | 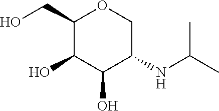 | >30 |
| A164 | 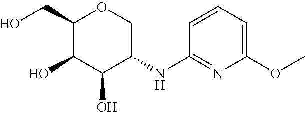 | +++ |
| A165 | 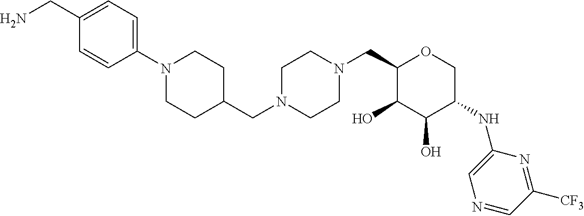 [SMILES: NCc1ccc(N2CCC(CN3CCN(C[C@H]4OC[C@H](Nc5cncc(C(F)(F)F)n5)[C@@H](O)[C@H]4O)CC3)CC2)cc1] | +++ |
| A166 | 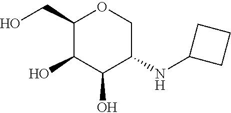 | >30 |
| A167 | 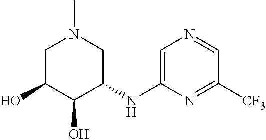 | |
| A168 | 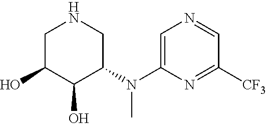 | |
| A169 | 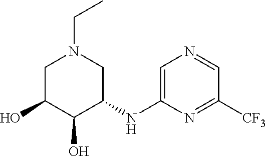 | |
| A170 | 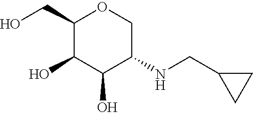 | |
| A171 | 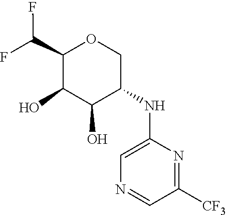 [SMILES: O[C@@H]1[C@H](O)[C@@H](Nc2cncc(C(F)(F)F)n2)CO[C@@H]1C(F)F] | |
| A172 | 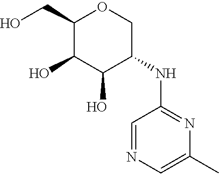 [SMILES: Cc1cncc(N[C@H]2CO[C@H](CO)[C@H](O)[C@@H]2O)n1] | |
| A173 | 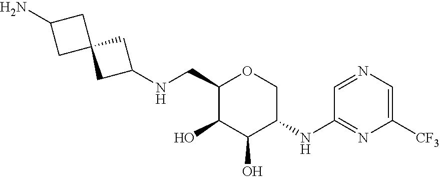 | |
| A174 | 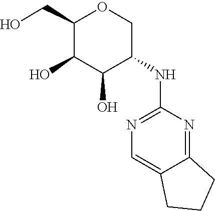 [SMILES: OC[C@H]1OC[C@H](Nc2ncc3c(n2)CCC3)[C@@H](O)[C@H]1O] | |
| A175 | 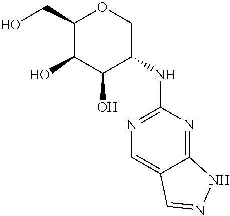 [SMILES: OC[C@H]1OC[C@H](Nc2ncc3cn[nH]c3n2)[C@@H](O)[C@H]1O] | |
| A176 | 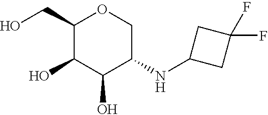 | |
| A177 | 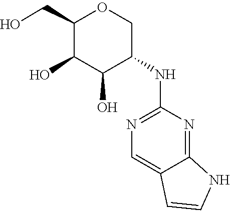 [SMILES: OC[C@H]1OC[C@H](Nc2ncc3cc[nH]c3n2)[C@@H](O)[C@H]1O] | |
| A178 | 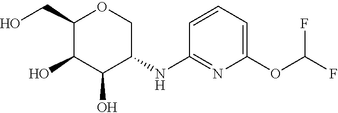 | |

In the table above, K_{d} values that are >=100 μM=+, <100 μM and >10 μM=++ and K 10 μM=+++

TABLE 1C
ASGPR Binding of Select Compounds

| Compound | | ASGPR |
|---|---|---|
| ID | Structure | K_{D} (μM) |
| A179 | 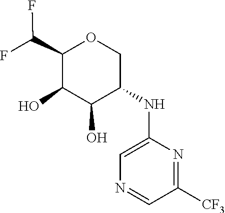 [SMILES: O[C@@H]1[C@H](O)[C@@H](Nc2cncc(C(F)(F)F)n2)CO[C@@H]1C(F)F] | +++ |
| A180 | 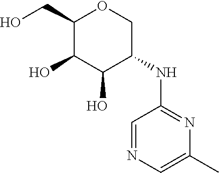 [SMILES: Cc1cncc(N[C@H]2CO[C@H](CO)[C@H](O)[C@@H]2O)n1] | +++ |
| A181 | 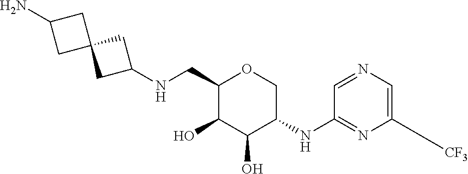 | |
| A182 | 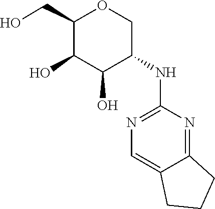 [SMILES: OC[C@H]1OC[C@H](Nc2ncc3c(n2)CCC3)[C@@H](O)[C@H]1O] | >30 |
| A183 | 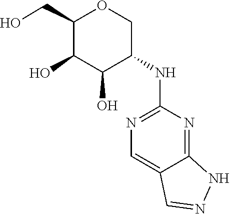 [SMILES: OC[C@H]1OC[C@H](Nc2ncc3cn[nH]c3n2)[C@@H](O)[C@H]1O] | >30 |
| A184 | 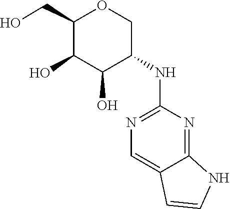 [SMILES: OC[C@H]1OC[C@H](Nc2ncc3cc[nH]c3n2)[C@@H](O)[C@H]1O] | >30 |
| A185 | 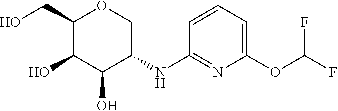 [SMILES: OC[C@H]1OC[C@H](Nc2cccc(OC(F)F)n2)[C@@H](O)[C@H]1O] | +++ |
| A186 | 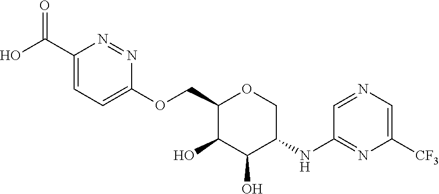 [SMILES: O=C(O)c1ccc(OC[C@H]2OC[C@H](Nc3cncc(C(F)(F)F)n3)[C@@H](O)[C@H]2O)nn1] | +++ |
| A187 | 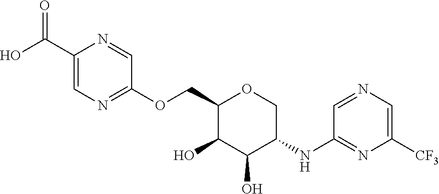 [SMILES: O=C(O)c1cnc(OC[C@H]2OC[C@H](Nc3cncc(C(F)(F)F)n3)[C@@H](O)[C@H]2O)cn1] | +++ |
| A188 | 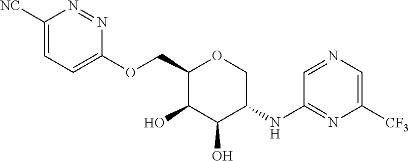 [SMILES: N#Cc1ccc(OC[C@H]2OC[C@H](Nc3cncc(C(F)(F)F)n3)[C@@H](O)[C@H]2O)nn1] | +++ |
| A189 | 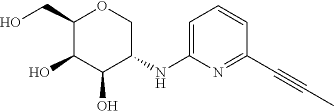 [SMILES: CC#Cc1cccc(N[C@H]2CO[C@H](CO)[C@H](O)[C@@H]2O)n1] | >30 |
| A190 | 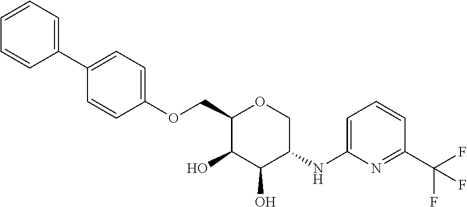 [SMILES: O[C@@H]1[C@H](O)[C@@H](Nc2cccc(C(F)(F)F)n2)CO[C@@H]1COc1ccc(-c2ccccc2)cc1] | +++ |
| A191 | 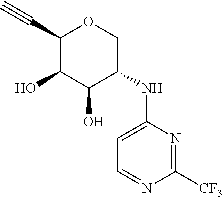 [SMILES: C#C[C@H]1OC[C@H](Nc2ccnc(C(F)(F)F)n2)[C@@H](O)[C@H]1O] | +++ |
| A192 | 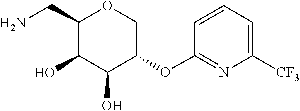 [SMILES: NC[C@H]1OC[C@H](Oc2cccc(C(F)(F)F)n2)[C@@H](O)[C@H]1O] | +++ |
| A193 | 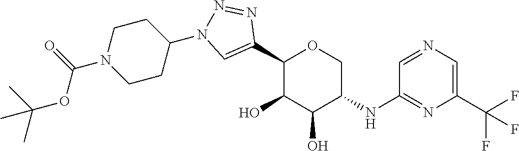 [SMILES: CC(C)(C)OC(=O)N1CCC(n2cc([C@H]3OC[C@H](Nc4cncc(C(F)(F)F)n4)[C@@H](O)[C@H]3O)nn2)CC1] | +++ |
| A194 | 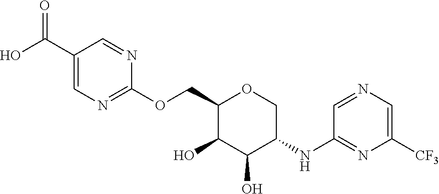 [SMILES: O=C(O)c1cnc(OC[C@H]2OC[C@H](Nc3cncc(C(F)(F)F)n3)[C@@H](O)[C@H]2O)nc1] | +++ |
| A195 | 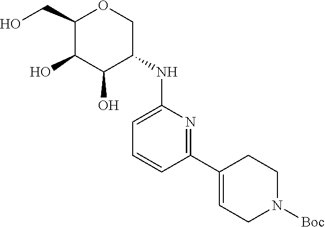 [SMILES: CC(C)(C)OC(=O)N1CC=C(c2cccc(N[C@H]3CO[C@H](CO)[C@H](O)[C@@H]3O)n2)CC1] | +++ |
| A196 | 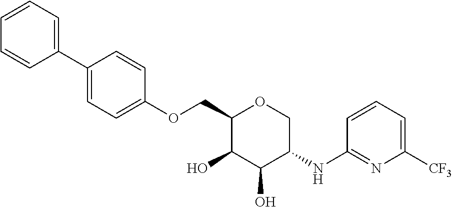 [SMILES: O[C@@H]1[C@H](O)[C@@H](Nc2cccc(C(F)(F)F)n2)CO[C@@H]1COc1ccc(-c2ccccc2)cc1] | +++ |
| A197 | 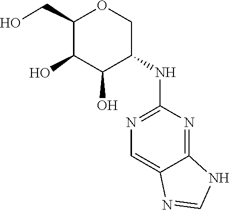 [SMILES: OC[C@H]1OC[C@H](Nc2ncc3nc[nH]c3n2)[C@@H](O)[C@H]1O] | >30 |
| A198 | 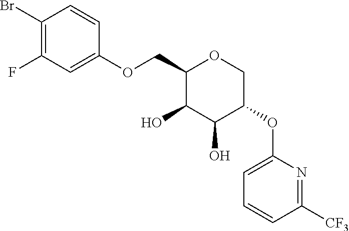 [SMILES: O[C@@H]1[C@H](O)[C@@H](Oc2cccc(C(F)(F)F)n2)CO[C@@H]1COc1ccc(Br)c(F)c1] | +++ |
| A199 | 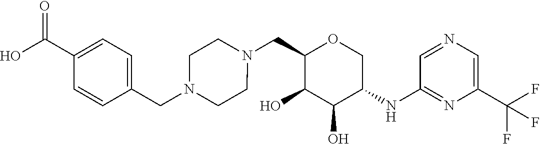 [SMILES: O=C(O)c1ccc(CN2CCN(C[C@H]3OC[C@H](Nc4cncc(C(F)(F)F)n4)[C@@H](O)[C@H]3O)CC2)cc1] | +++ |
| A200 | 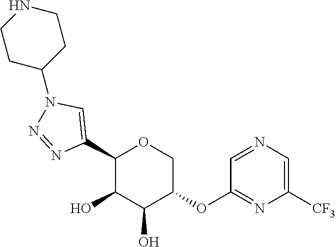 [SMILES: O[C@@H]1[C@H](O)[C@@H](Oc2cncc(C(F)(F)F)n2)CO[C@@H]1c1cn(C2CCNCC2)nn1] | +++ |
| A201 | 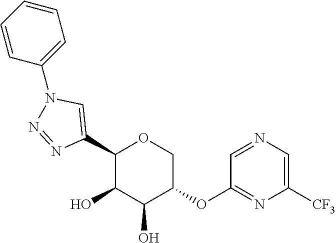 [SMILES: O[C@@H]1[C@H](O)[C@@H](Oc2cncc(C(F)(F)F)n2)CO[C@@H]1c1cn(-c2ccccc2)nn1] | +++ |
| A202 | 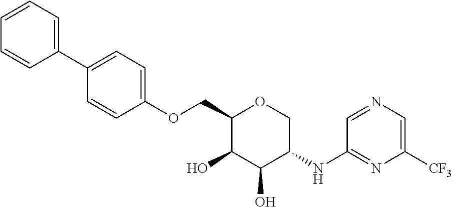 [SMILES: O[C@@H]1[C@H](O)[C@@H](Nc2cncc(C(F)(F)F)n2)CO[C@@H]1COc1ccc(-c2ccccc2)cc1] | +++ |
| A203 | 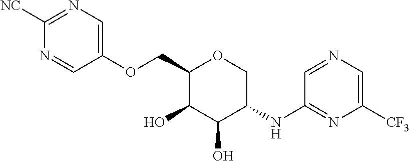 [SMILES: N#Cc1ncc(OC[C@H]2OC[C@H](Nc3cncc(C(F)(F)F)n3)[C@@H](O)[C@H]2O)cn1] | +++ |
| A204 | 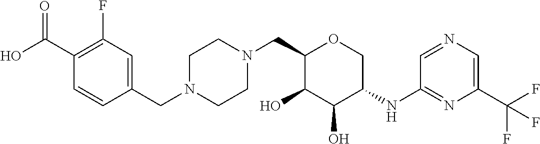 [SMILES: O=C(O)c1ccc(CN2CCN(C[C@H]3OC[C@H](Nc4cncc(C(F)(F)F)n4)[C@@H](O)[C@H]3O)CC2)cc1F] | +++ |
| A205 | 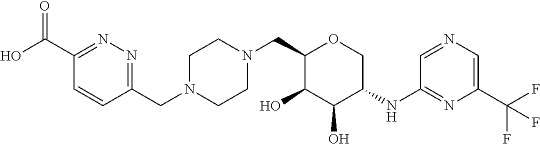 [SMILES: O=C(O)c1ccc(CN2CCN(C[C@H]3OC[C@H](Nc4cncc(C(F)(F)F)n4)[C@@H](O)[C@H]3O)CC2)nn1] | +++ |
| A206 | 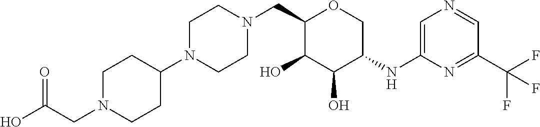 [SMILES: O=C(O)CN1CCC(N2CCN(C[C@H]3OC[C@H](Nc4cncc(C(F)(F)F)n4)[C@@H](O)[C@H]3O)CC2)CC1] | +++ |
| A207 | 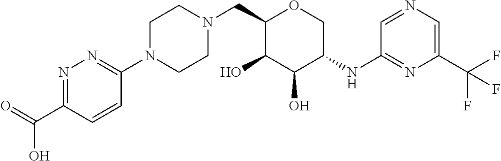 [SMILES: O=C(O)c1ccc(N2CCN(C[C@H]3OC[C@H](Nc4cncc(C(F)(F)F)n4)[C@@H](O)[C@H]3O)CC2)nn1] | +++ |
| A208 | 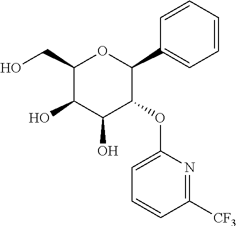 [SMILES: OC[C@H]1O[C@@H](c2ccccc2)[C@H](Oc2cccc(C(F)(F)F)n2)[C@@H](O)[C@H]1O] | >30 |
| A209 | 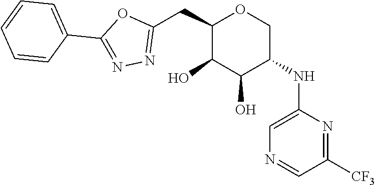 [SMILES: O[C@@H]1[C@H](O)[C@@H](Nc2cncc(C(F)(F)F)n2)CO[C@@H]1Cc1nnc(-c2ccccc2)o1] | +++ |
| A210 | 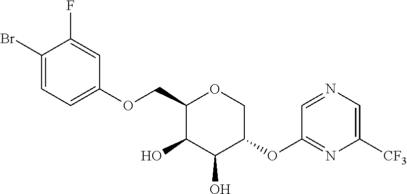 [SMILES: O[C@@H]1[C@H](O)[C@@H](Oc2cncc(C(F)(F)F)n2)CO[C@@H]1COc1ccc(Br)c(F)c1] | +++ |
| A211 | 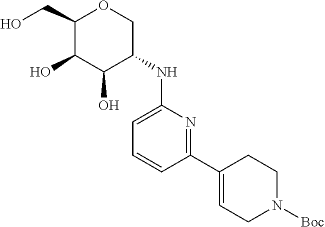 [SMILES: CC(C)(C)OC(=O)N1CC=C(c2cccc(N[C@H]3CO[C@H](CO)[C@H](O)[C@@H]3O)n2)CC1] | >30 |
| A212 | 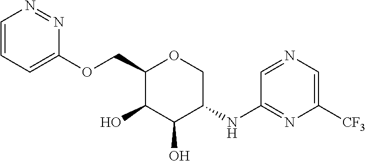 [SMILES: O[C@@H]1[C@H](O)[C@@H](Nc2cncc(C(F)(F)F)n2)CO[C@@H]1COc1cccnn1] | +++ |
| A213 | 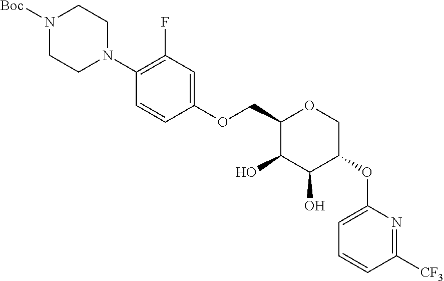 [SMILES: CC(C)(C)OC(=O)N1CCN(c2ccc(OC[C@H]3OC[C@H](Oc4cccc(C(F)(F)F)n4)[C@@H](O)[C@H]3O)cc2F)CC1] | +++ |
| A214 | 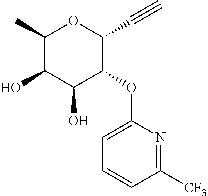 [SMILES: C#C[C@H]1O[C@H](C)[C@H](O)[C@H](O)[C@H]1Oc1cccc(C(F)(F)F)n1] | +++ |
| A215 | 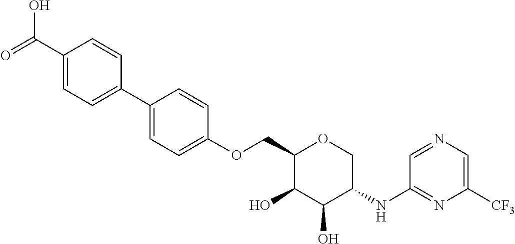 [SMILES: O=C(O)c1ccc(-c2ccc(OC[C@H]3OC[C@H](Nc4cncc(C(F)(F)F)n4)[C@@H](O)[C@H]3O)cc2)cc1] | +++ |
| A216 | 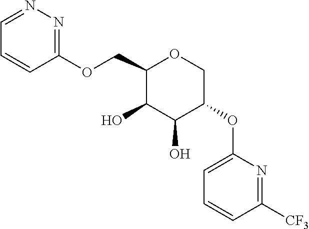 [SMILES: O[C@@H]1[C@H](O)[C@@H](Oc2cccc(C(F)(F)F)n2)CO[C@@H]1COc1cccnn1] | +++ |
| A217 | 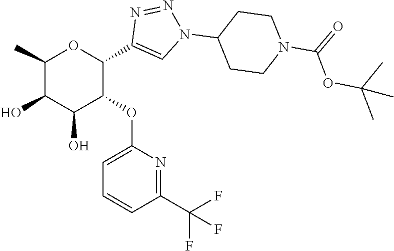 [SMILES: C[C@H]1O[C@H](c2cn(C3CCN(C(=O)OC(C)(C)C)CC3)nn2)[C@H](Oc2cccc(C(F)(F)F)n2)[C@@H](O)[C@H]1O] | +++ |
| A218 | 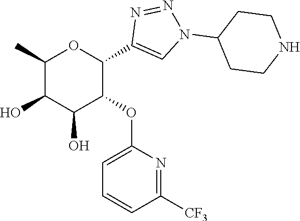 [SMILES: C[C@H]1O[C@H](c2cn(C3CCNCC3)nn2)[C@H](Oc2cccc(C(F)(F)F)n2)[C@@H](O)[C@H]1O] | +++ |
| A219 | 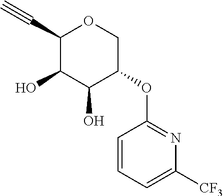 [SMILES: C#C[C@H]1OC[C@H](Oc2cccc(C(F)(F)F)n2)[C@@H](O)[C@H]1O] | +++ |
| A220 | 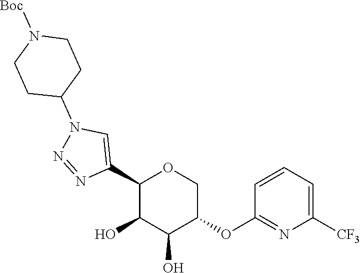 [SMILES: CC(C)(C)OC(=O)N1CCC(n2cc([C@H]3OC[C@H](Oc4cccc(C(F)(F)F)n4)[C@@H](O)[C@H]3O)nn2)CC1] | +++ |
| A221 | 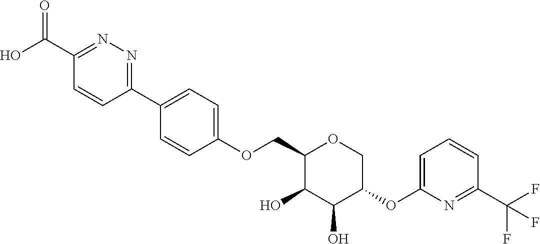 [SMILES: O=C(O)c1ccc(-c2ccc(OC[C@H]3OC[C@H](Oc4cccc(C(F)(F)F)n4)[C@@H](O)[C@H]3O)cc2)nn1] | +++ |
| A222 | 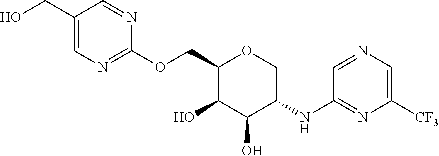 [SMILES: OCc1cnc(OC[C@H]2OC[C@H](Nc3cncc(C(F)(F)F)n3)[C@@H](O)[C@H]2O)nc1] | +++ |
| A223 | 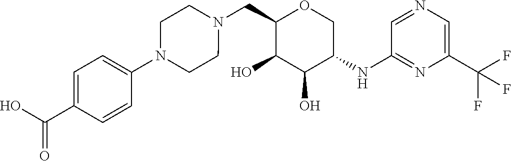 [SMILES: O=C(O)c1ccc(N2CCN(C[C@H]3OC[C@H](Nc4cncc(C(F)(F)F)n4)[C@@H](O)[C@H]3O)CC2)cc1] | +++ |
| A224 | 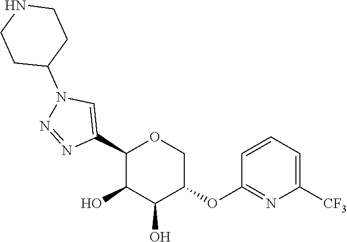 [SMILES: O[C@@H]1[C@H](O)[C@@H](Oc2cccc(C(F)(F)F)n2)CO[C@@H]1c1cn(C2CCNCC2)nn1] | +++ |
| A225 | 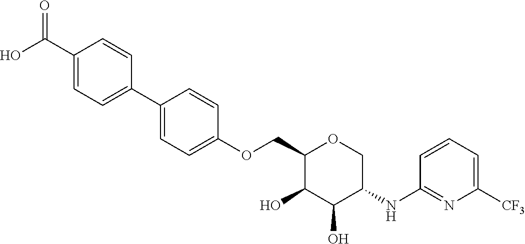 [SMILES: O=C(O)c1ccc(-c2ccc(OC[C@H]3OC[C@H](Nc4cccc(C(F)(F)F)n4)[C@@H](O)[C@H]3O)cc2)cc1] | +++ |
| A226 | 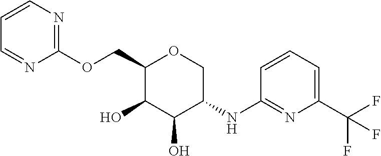 [SMILES: O[C@@H]1[C@H](O)[C@@H](Nc2cccc(C(F)(F)F)n2)CO[C@@H]1COc1ncccn1] | +++ |
| A227 | 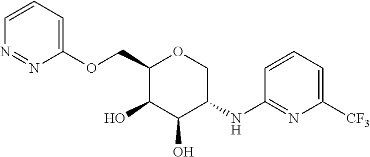 [SMILES: O[C@@H]1[C@H](O)[C@@H](Nc2cccc(C(F)(F)F)n2)CO[C@@H]1COc1cccnn1] | +++ |
| A228 | 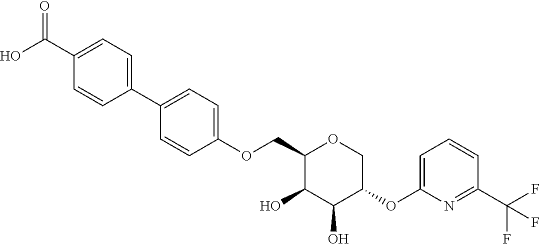 [SMILES: O=C(O)c1ccc(-c2ccc(OC[C@H]3OC[C@H](Oc4cccc(C(F)(F)F)n4)[C@@H](O)[C@H]3O)cc2)cc1] | +++ |
| A229 | 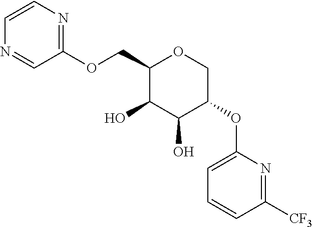 [SMILES: O[C@@H]1[C@H](O)[C@@H](Oc2cccc(C(F)(F)F)n2)CO[C@@H]1COc1cnccn1] | +++ |
| A230 | 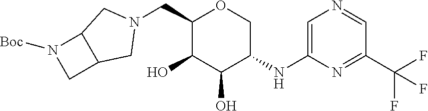 [SMILES: CC(C)(C)OC(=O)N1CC2CN(C[C@H]3OC[C@H](Nc4cncc(C(F)(F)F)n4)[C@@H](O)[C@H]3O)CC21] | +++ |
| A231 | 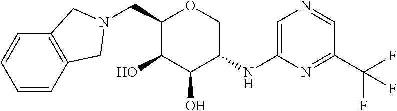 [SMILES: O[C@@H]1[C@H](O)[C@@H](Nc2cncc(C(F)(F)F)n2)CO[C@@H]1CN1Cc2ccccc2C1] | +++ |
| A232 | 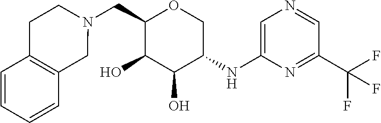 [SMILES: O[C@@H]1[C@H](O)[C@@H](Nc2cncc(C(F)(F)F)n2)CO[C@@H]1CN1CCc2ccccc2C1] | +++ |
| A233 | 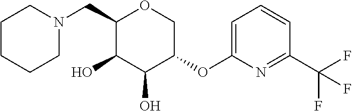 [SMILES: O[C@@H]1[C@H](O)[C@@H](Oc2cccc(C(F)(F)F)n2)CO[C@@H]1CN1CCCCC1] | +++ |
| A234 | 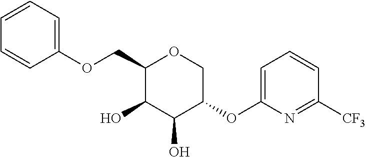 [SMILES: O[C@@H]1[C@H](O)[C@@H](Oc2cccc(C(F)(F)F)n2)CO[C@@H]1COc1ccccc1] | +++ |
| A235 | 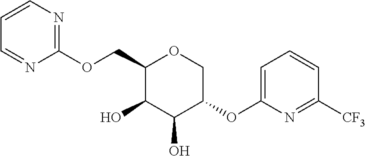 [SMILES: O[C@@H]1[C@H](O)[C@@H](Oc2cccc(C(F)(F)F)n2)CO[C@@H]1COc1ncccn1] | +++ |
| A236 | 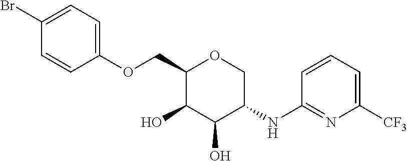 [SMILES: O[C@@H]1[C@H](O)[C@@H](Nc2cccc(C(F)(F)F)n2)CO[C@@H]1COc1ccc(Br)cc1] | +++ |
| A237 | 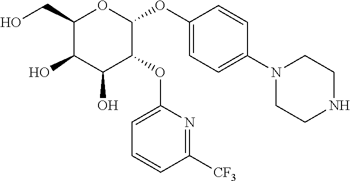 [SMILES: OC[C@H]1O[C@H](Oc2ccc(N3CCNCC3)cc2)[C@H](Oc2cccc(C(F)(F)F)n2)[C@@H](O)[C@H]1O] | +++ |
| A238 | 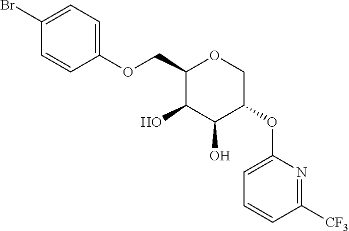 [SMILES: O[C@@H]1[C@H](O)[C@@H](Oc2cccc(C(F)(F)F)n2)CO[C@@H]1COc1ccc(Br)cc1] | +++ |
| A239 | 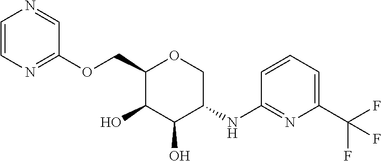 [SMILES: O[C@@H]1[C@H](O)[C@@H](Nc2cccc(C(F)(F)F)n2)CO[C@@H]1COc1cnccn1] | +++ |
| A240 | 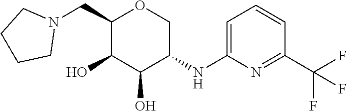 [SMILES: O[C@@H]1[C@H](O)[C@@H](Nc2cccc(C(F)(F)F)n2)CO[C@@H]1CN1CCCC1] | +++ |
| A241 | 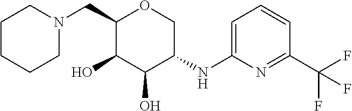 [SMILES: O[C@@H]1[C@H](O)[C@@H](Nc2cccc(C(F)(F)F)n2)CO[C@@H]1CN1CCCCC1] | +++ |
| A242 | 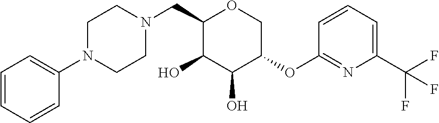 [SMILES: O[C@@H]1[C@H](O)[C@@H](Oc2cccc(C(F)(F)F)n2)CO[C@@H]1CN1CCN(c2ccccc2)CC1] | +++ |
| A243 | 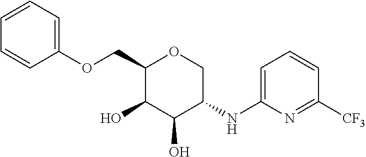 [SMILES: O[C@@H]1[C@H](O)[C@@H](Nc2cccc(C(F)(F)F)n2)CO[C@@H]1COc1ccccc1] | +++ |
| A244 | 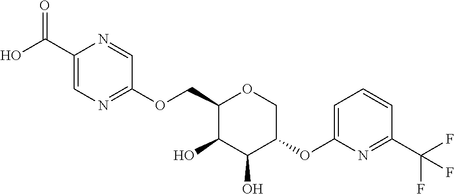 [SMILES: O=C(O)c1cnc(OC[C@H]2OC[C@H](Oc3cccc(C(F)(F)F)n3)[C@@H](O)[C@H]2O)cn1] | +++ |
| A245 | 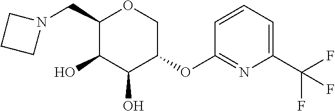 [SMILES: O[C@@H]1[C@H](O)[C@@H](Oc2cccc(C(F)(F)F)n2)CO[C@@H]1CN1CCC1] | +++ |
| A246 | 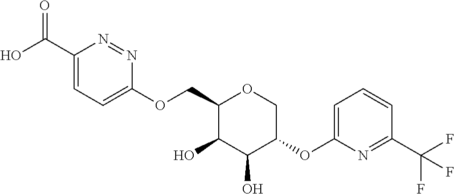 [SMILES: O=C(O)c1ccc(OC[C@H]2OC[C@H](Oc3cccc(C(F)(F)F)n3)[C@@H](O)[C@H]2O)nn1] | +++ |
| A247 | 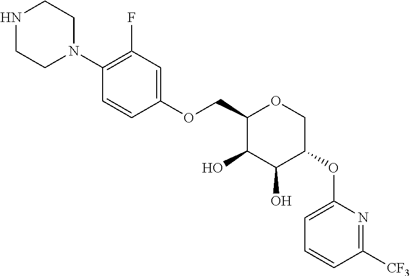 [SMILES: O[C@@H]1[C@H](O)[C@@H](Oc2cccc(C(F)(F)F)n2)CO[C@@H]1COc1ccc(N2CCNCC2)c(F)c1] | +++ |
| A248 | 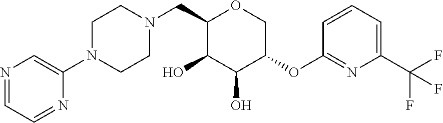 [SMILES: O[C@@H]1[C@H](O)[C@@H](Oc2cccc(C(F)(F)F)n2)CO[C@@H]1CN1CCN(c2cnccn2)CC1] | +++ |
| A249 | 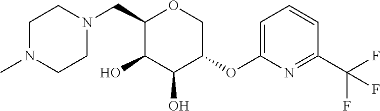 [SMILES: CN1CCN(C[C@H]2OC[C@H](Oc3cccc(C(F)(F)F)n3)[C@@H](O)[C@H]2O)CC1] | +++ |
| A250 | 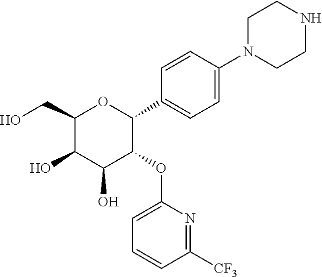 [SMILES: OC[C@H]1O[C@H](c2ccc(N3CCNCC3)cc2)[C@H](Oc2cccc(C(F)(F)F)n2)[C@@H](O)[C@H]1O] | +++ |
| A251 | 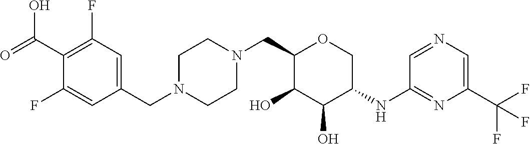 [SMILES: O=C(O)c1c(F)cc(CN2CCN(C[C@H]3OC[C@H](Nc4cncc(C(F)(F)F)n4)[C@@H](O)[C@H]3O)CC2)cc1F] | +++ |
| A252 | 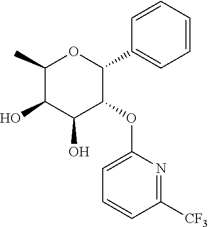 [SMILES: C[C@H]1O[C@H](c2ccccc2)[C@H](Oc2cccc(C(F)(F)F)n2)[C@@H](O)[C@H]1O] | +++ |
| A253 | 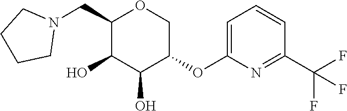 [SMILES: O[C@@H]1[C@H](O)[C@@H](Oc2cccc(C(F)(F)F)n2)CO[C@@H]1CN1CCCC1] | |
| A254 | 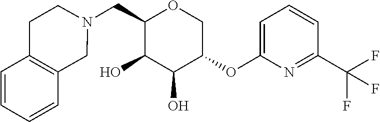 [SMILES: O[C@@H]1[C@H](O)[C@@H](Oc2cccc(C(F)(F)F)n2)CO[C@@H]1CN1CCc2ccccc2C1] | +++ |
| A255 | 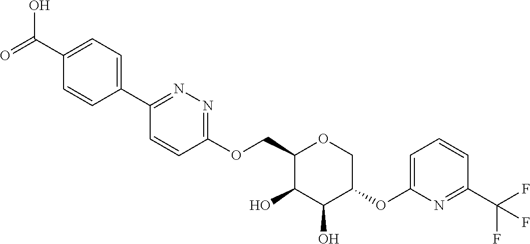 [SMILES: O=C(O)c1ccc(-c2ccc(OC[C@H]3OC[C@H](Oc4cccc(C(F)(F)F)n4)[C@@H](O)[C@H]3O)nn2)cc1] | +++ |
| A256 | 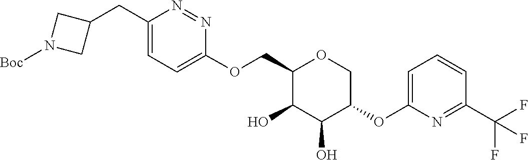 [SMILES: CC(C)(C)OC(=O)N1CC(Cc2ccc(OC[C@H]3OC[C@H](Oc4cccc(C(F)(F)F)n4)[C@@H](O)[C@H]3O)nn2)C1] | +++ |
| A257 | 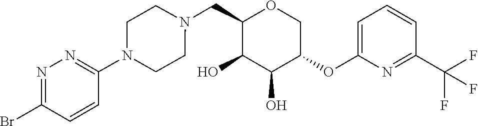 [SMILES: O[C@@H]1[C@H](O)[C@@H](Oc2cccc(C(F)(F)F)n2)CO[C@@H]1CN1CCN(c2ccc(Br)nn2)CC1] | +++ |
| A258 | 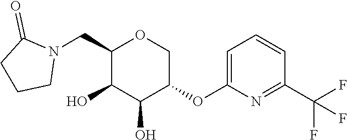 [SMILES: O=C1CCCN1C[C@H]1OC[C@H](Oc2cccc(C(F)(F)F)n2)[C@@H](O)[C@H]1O] | +++ |
| A259 | 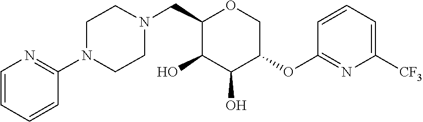 [SMILES: O[C@@H]1[C@H](O)[C@@H](Oc2cccc(C(F)(F)F)n2)CO[C@@H]1CN1CCN(c2ccccn2)CC1] | +++ |
| A260 | 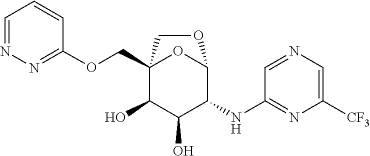 [SMILES: O[C@@H]1[C@@H](Nc2cncc(C(F)(F)F)n2)[C@H]2OC[C@](COc3cccnn3)(O2)[C@@H]1O] | +++ |
| A261 | 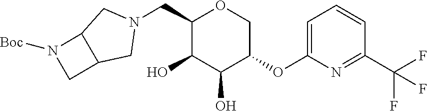 [SMILES: CC(C)(C)OC(=O)N1CC2CN(C[C@H]3OC[C@H](Oc4cccc(C(F)(F)F)n4)[C@@H](O)[C@H]3O)CC21] | +++ |
| A262 | 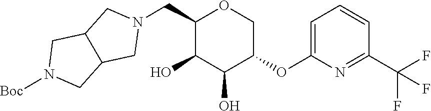 [SMILES: CC(C)(C)OC(=O)N1CC2CN(C[C@H]3OC[C@H](Oc4cccc(C(F)(F)F)n4)[C@@H](O)[C@H]3O)CC2C1] | +++ |
| A263 | 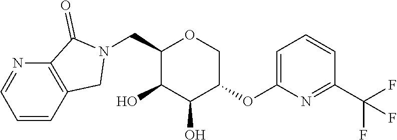 [SMILES: O=C1c2ncccc2CN1C[C@H]1OC[C@H](Oc2cccc(C(F)(F)F)n2)[C@@H](O)[C@H]1O] | +++ |
| A264 | 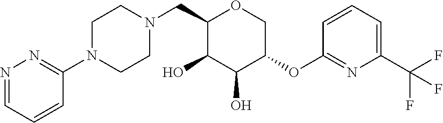 [SMILES: O[C@@H]1[C@H](O)[C@@H](Oc2cccc(C(F)(F)F)n2)CO[C@@H]1CN1CCN(c2cccnn2)CC1] | +++ |
| A265 | 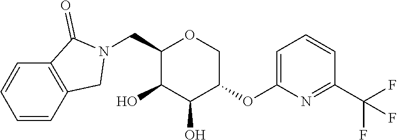 [SMILES: O=C1c2ccccc2CN1C[C@H]1OC[C@H](Oc2cccc(C(F)(F)F)n2)[C@@H](O)[C@H]1O] | +++ |
| A267 | 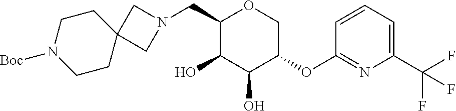 [SMILES: CC(C)(C)OC(=O)N1CCC2(CC1)CN(C[C@H]1OC[C@H](Oc3cccc(C(F)(F)F)n3)[C@@H](O)[C@H]1O)C2] | +++ |
| A268 | 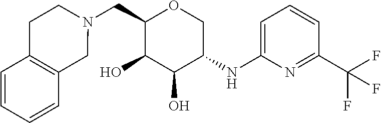 [SMILES: O[C@@H]1[C@H](O)[C@@H](Nc2cccc(C(F)(F)F)n2)CO[C@@H]1CN1CCc2ccccc2C1] | +++ |
| A269 | 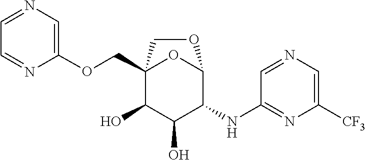 [SMILES: O[C@@H]1[C@@H](Nc2cncc(C(F)(F)F)n2)[C@H]2OC[C@](COc3cnccn3)(O2)[C@@H]1O] | +++ |
| A270 | 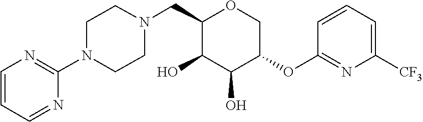 [SMILES: O[C@@H]1[C@H](O)[C@@H](Oc2cccc(C(F)(F)F)n2)CO[C@@H]1CN1CCN(c2ncccn2)CC1] | +++ |
| A271 | 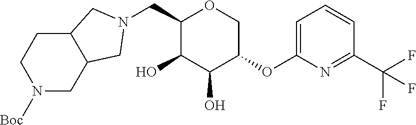 [SMILES: CC(C)(C)OC(=O)N1CCC2CN(C[C@H]3OC[C@H](Oc4cccc(C(F)(F)F)n4)[C@@H](O)[C@H]3O)CC2C1] | +++ |
| A272 | 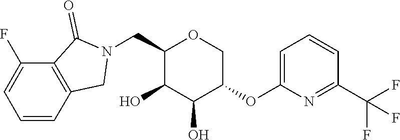 [SMILES: O=C1c2c(F)cccc2CN1C[C@H]1OC[C@H](Oc2cccc(C(F)(F)F)n2)[C@@H](O)[C@H]1O] | +++ |
| A273 | 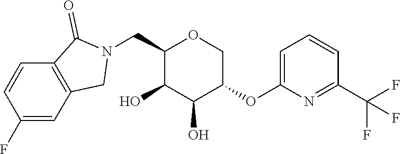 [SMILES: O=C1c2ccc(F)cc2CN1C[C@H]1OC[C@H](Oc2cccc(C(F)(F)F)n2)[C@@H](O)[C@H]1O] | +++ |
| A274 | 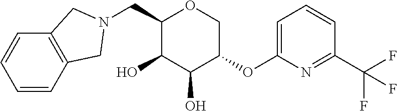 [SMILES: O[C@@H]1[C@H](O)[C@@H](Oc2cccc(C(F)(F)F)n2)CO[C@@H]1CN1Cc2ccccc2C1] | +++ |
| A275 | 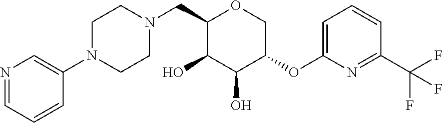 [SMILES: O[C@@H]1[C@H](O)[C@@H](Oc2cccc(C(F)(F)F)n2)CO[C@@H]1CN1CCN(c2cccnc2)CC1] | +++ |
| A276 | 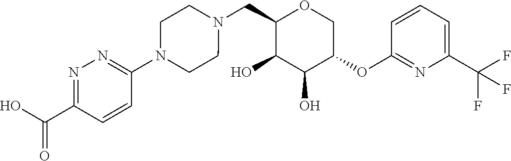 [SMILES: O=C(O)c1ccc(N2CCN(C[C@H]3OC[C@H](Oc4cccc(C(F)(F)F)n4)[C@@H](O)[C@H]3O)CC2)nn1] | +++ |
| A277 | 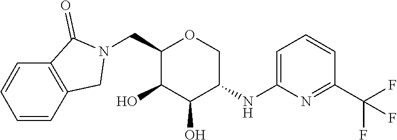 [SMILES: O=C1c2ccccc2CN1C[C@H]1OC[C@H](Nc2cccc(C(F)(F)F)n2)[C@@H](O)[C@H]1O] | +++ |
| A279 | 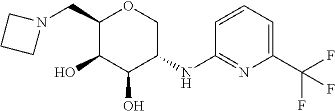 [SMILES: O[C@@H]1[C@H](O)[C@@H](Nc2cccc(C(F)(F)F)n2)CO[C@@H]1CN1CCC1] | +++ |
| A280 | 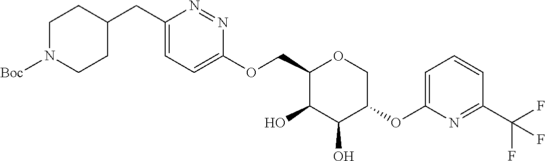 [SMILES: CC(C)(C)OC(=O)N1CCC(Cc2ccc(OC[C@H]3OC[C@H](Oc4cccc(C(F)(F)F)n4)[C@@H](O)[C@H]3O)nn2)CC1] | +++ |
| A281 | 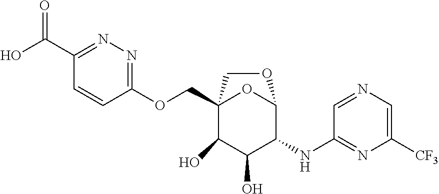 [SMILES: O=C(O)c1ccc(OC[C@@]23CO[C@@H](O2)[C@H](Nc2cncc(C(F)(F)F)n2)[C@@H](O)[C@H]3O)nn1] | +++ |
| A284 | 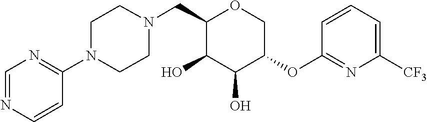 [SMILES: O[C@@H]1[C@H](O)[C@@H](Oc2cccc(C(F)(F)F)n2)CO[C@@H]1CN1CCN(c2ccncn2)CC1] | +++ |
| A285 | 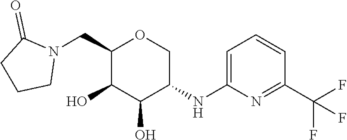 [SMILES: O=C1CCCN1C[C@H]1OC[C@H](Nc2cccc(C(F)(F)F)n2)[C@@H](O)[C@H]1O] | +++ |
| A286 | 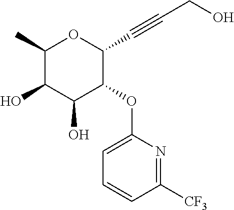 [SMILES: C[C@H]1O[C@H](C#CCO)[C@H](Oc2cccc(C(F)(F)F)n2)[C@@H](O)[C@H]1O] | +++ |
| A287 | 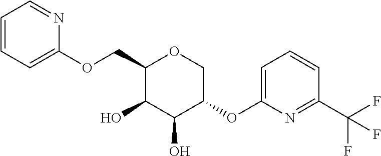 [SMILES: O[C@@H]1[C@H](O)[C@@H](Oc2cccc(C(F)(F)F)n2)CO[C@@H]1COc1ccccn1] | +++ |
| A288 | 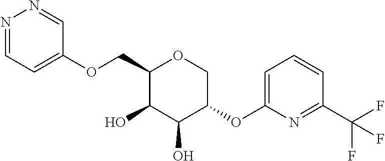 [SMILES: O[C@@H]1[C@H](O)[C@@H](Oc2cccc(C(F)(F)F)n2)CO[C@@H]1COc1ccnnc1] | +++ |
| A289 | 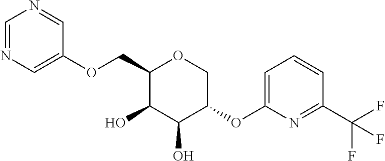 [SMILES: O[C@@H]1[C@H](O)[C@@H](Oc2cccc(C(F)(F)F)n2)CO[C@@H]1COc1cncnc1] | +++ |
| A290 | 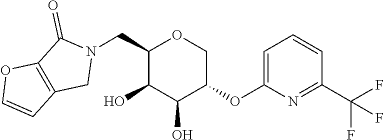 [SMILES: O=C1c2occc2CN1C[C@H]1OC[C@H](Oc2cccc(C(F)(F)F)n2)[C@@H](O)[C@H]1O] | +++ |
| A291 | 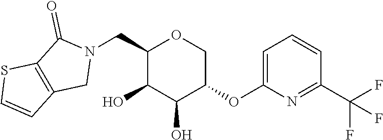 [SMILES: O=C1c2sccc2CN1C[C@H]1OC[C@H](Oc2cccc(C(F)(F)F)n2)[C@@H](O)[C@H]1O] | +++ |
| A292 | 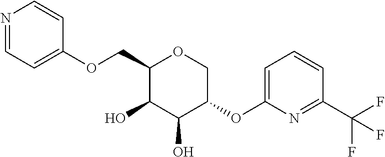 [SMILES: O[C@@H]1[C@H](O)[C@@H](Oc2cccc(C(F)(F)F)n2)CO[C@@H]1COc1ccncc1] | +++ |
| A293 | 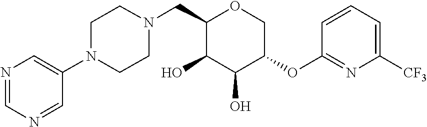 [SMILES: O[C@@H]1[C@H](O)[C@@H](Oc2cccc(C(F)(F)F)n2)CO[C@@H]1CN1CCN(c2cncnc2)CC1] | +++ |
| A294 | 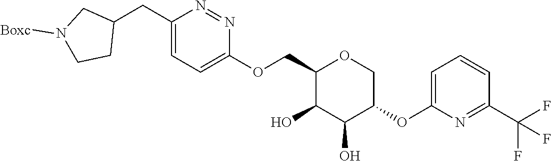 | +++ |
| A295 | 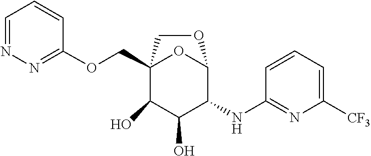 [SMILES: O[C@@H]1[C@@H](Nc2cccc(C(F)(F)F)n2)[C@H]2OC[C@](COc3cccnn3)(O2)[C@@H]1O] | +++ |
| A296 | 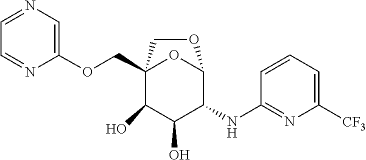 [SMILES: O[C@@H]1[C@@H](Nc2cccc(C(F)(F)F)n2)[C@H]2OC[C@](COc3cnccn3)(O2)[C@@H]1O] | +++ |
| A297 | 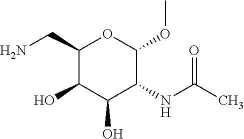 [SMILES: CO[C@H]1O[C@H](CN)[C@H](O)[C@H](O)[C@H]1NC(C)=O] | |
| A298 | 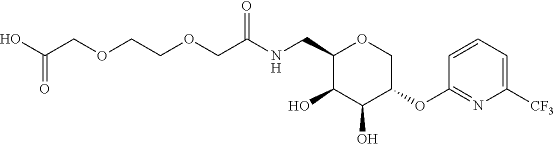 [SMILES: O=C(O)COCCOCC(=O)NC[C@H]1OC[C@H](Oc2cccc(C(F)(F)F)n2)[C@@H](O)[C@H]1O] | +++ |
| A299 | 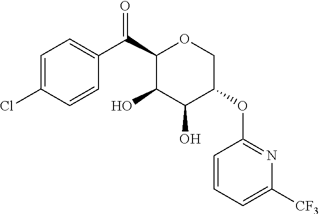 [SMILES: O=C(c1ccc(Cl)cc1)[C@H]1OC[C@H](Oc2cccc(C(F)(F)F)n2)[C@@H](O)[C@H]1O] | +++ |
| A300 | 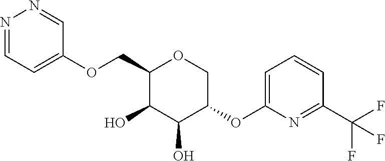 [SMILES: O[C@@H]1[C@H](O)[C@@H](Oc2cccc(C(F)(F)F)n2)CO[C@@H]1COc1ccnnc1] | +++ |
| A301 | 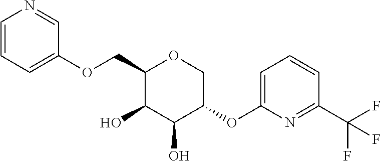 [SMILES: O[C@@H]1[C@H](O)[C@@H](Oc2cccc(C(F)(F)F)n2)CO[C@@H]1COc1cccnc1] | +++ |
| A302 | 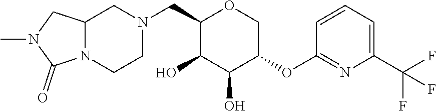 [SMILES: CN1CC2CN(C[C@H]3OC[C@H](Oc4cccc(C(F)(F)F)n4)[C@@H](O)[C@H]3O)CCN2C1=O] | +++ |
| A303 | 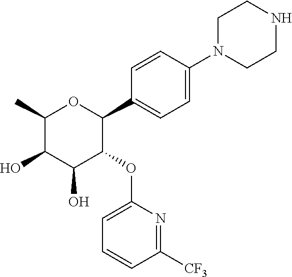 [SMILES: C[C@H]1O[C@@H](c2ccc(N3CCNCC3)cc2)[C@H](Oc2cccc(C(F)(F)F)n2)[C@@H](O)[C@H]1O] | +++ |
| A304 | 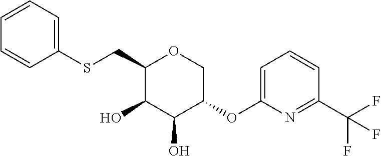 [SMILES: O[C@@H]1[C@H](O)[C@@H](Oc2cccc(C(F)(F)F)n2)CO[C@@H]1CSc1ccccc1] | +++ |
| A305 | 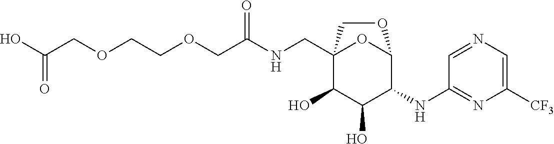 [SMILES: O=C(O)COCCOCC(=O)NC[C@@]12CO[C@@H](O1)[C@H](Nc1cncc(C(F)(F)F)n1)[C@@H](O)[C@H]2O] | +++ |
| A306 | 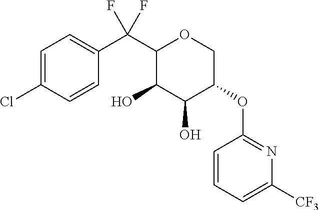 [SMILES: O[C@@H]1[C@@H](Oc2cccc(C(F)(F)F)n2)COC(C(F)(F)c2ccc(Cl)cc2)[C@@H]1O] | +++ |
| A307 | 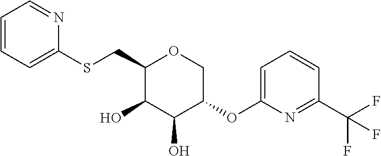 [SMILES: O[C@@H]1[C@H](O)[C@@H](Oc2cccc(C(F)(F)F)n2)CO[C@@H]1CSc1ccccn1] | +++ |
| A308 | 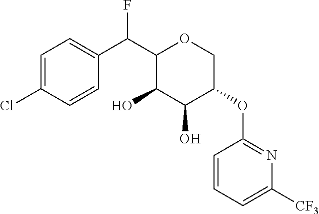 [SMILES: O[C@@H]1[C@@H](Oc2cccc(C(F)(F)F)n2)COC(C(F)c2ccc(Cl)cc2)[C@@H]1O] | +++ |
| A309 | 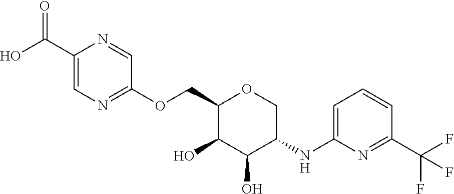 [SMILES: O=C(O)c1cnc(OC[C@H]2OC[C@H](Nc3cccc(C(F)(F)F)n3)[C@@H](O)[C@H]2O)cn1] | +++ |
| A310 | 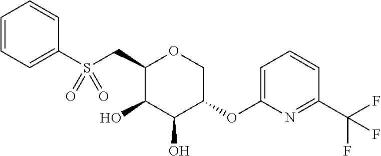 [SMILES: O=S(=O)(C[C@H]1OC[C@H](Oc2cccc(C(F)(F)F)n2)[C@@H](O)[C@H]1O)c1ccccc1] | +++ |
| A311 | 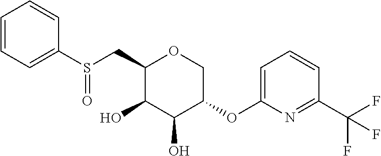 [SMILES: O=S(C[C@H]1OC[C@H](Oc2cccc(C(F)(F)F)n2)[C@@H](O)[C@H]1O)c1ccccc1] | +++ |
| A312 | 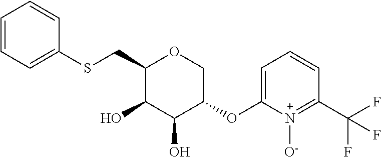 [SMILES: [O-][n+]1c(O[C@H]2CO[C@H](CSc3ccccc3)[C@H](O)[C@@H]2O)cccc1C(F)(F)F] | +++ |
| A313 | 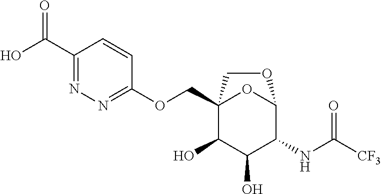 [SMILES: CC[C@]1(COc2ccc(C(=O)O)nn2)O[C@H](O)[C@H](NC(=O)C(F)(F)F)[C@@H](O)[C@H]1O] | +++ |
| A314 | 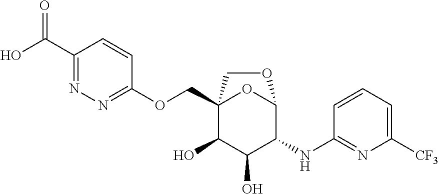 [SMILES: CC[C@]1(COc2ccc(C(=O)O)nn2)O[C@H](O)[C@H](Nc2cccc(C(F)(F)F)n2)[C@@H](O)[C@H]1O] | +++ |
| A315 | 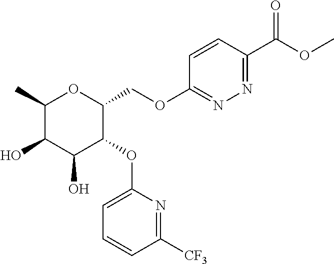 | +++ |
| A316 | 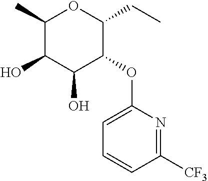 [SMILES: CC[C@H]1O[C@H](C)[C@H](O)[C@H](O)[C@H]1Oc1cccc(C(F)(F)F)n1] | +++ |
| A317 | 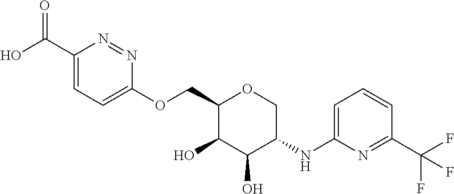 [SMILES: O=C(O)c1ccc(OC[C@H]2OC[C@H](Nc3cccc(C(F)(F)F)n3)[C@@H](O)[C@H]2O)nn1] | +++ |
| A318 | 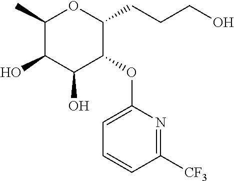 [SMILES: C[C@H]1O[C@H](CCCO)[C@H](Oc2cccc(C(F)(F)F)n2)[C@@H](O)[C@H]1O] | +++ |
| A319 | 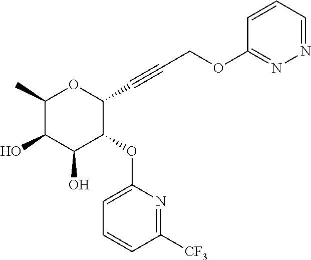 [SMILES: C[C@H]1O[C@H](C#CCOc2cccnn2)[C@H](Oc2cccc(C(F)(F)F)n2)[C@@H](O)[C@H]1O] | +++ |
| A321 | 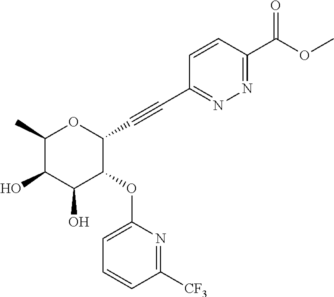 [SMILES: COC(=O)c1ccc(C#C[C@H]2O[C@H](C)[C@H](O)[C@H](O)[C@H]2Oc2cccc(C(F)(F)F)n2)nn1] | +++ |
| A322 | 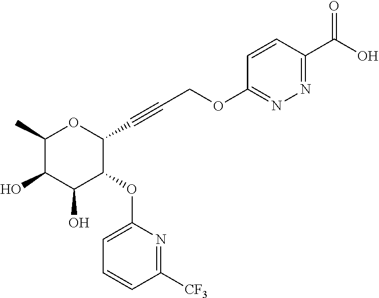 [SMILES: C[C@H]1O[C@H](C#CCOc2ccc(C(=O)O)nn2)[C@H](Oc2cccc(C(F)(F)F)n2)[C@@H](O)[C@H]1O] | +++ |
| A323 | 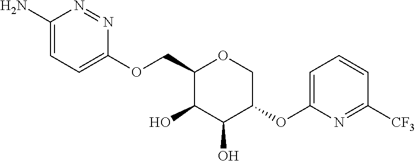 [SMILES: Nc1ccc(OC[C@H]2OC[C@H](Oc3cccc(C(F)(F)F)n3)[C@@H](O)[C@H]2O)nn1] | +++ |
| A325 | 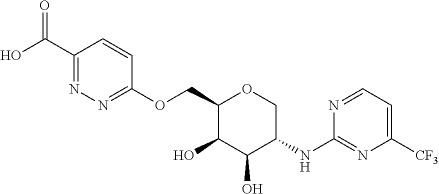 [SMILES: O=C(O)c1ccc(OC[C@H]2OC[C@H](Nc3nccc(C(F)(F)F)n3)[C@@H](O)[C@H]2O)nn1] | +++ |
| A326 | 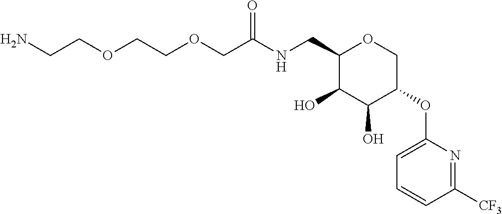 [SMILES: NCCOCCOCC(=O)NC[C@H]1OC[C@H](Oc2cccc(C(F)(F)F)n2)[C@@H](O)[C@H]1O] | +++ |
| A329 | 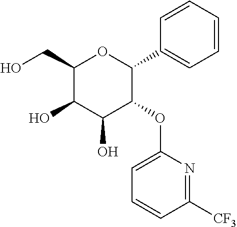 [SMILES: OC[C@H]1O[C@H](c2ccccc2)[C@H](Oc2cccc(C(F)(F)F)n2)[C@@H](O)[C@H]1O] | +++ |
| A330 | 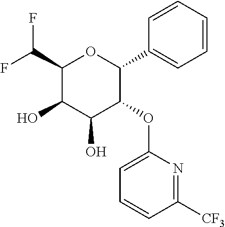 [SMILES: O[C@@H]1[C@H](O)[C@@H](Oc2cccc(C(F)(F)F)n2)[C@@H](c2ccccc2)O[C@@H]1C(F)F] | +++ |
| A331 | 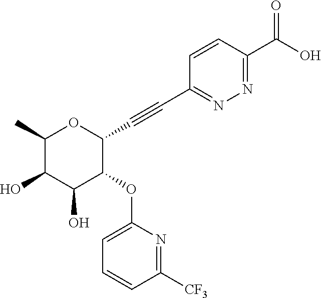 [SMILES: C[C@H]1O[C@H](C#Cc2ccc(C(=O)O)nn2)[C@H](Oc2cccc(C(F)(F)F)n2)[C@@H](O)[C@H]1O] | +++ |
| A332 | 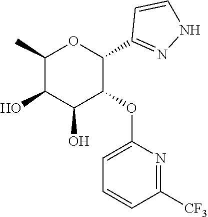 [SMILES: C[C@H]1O[C@H](c2cc[nH]n2)[C@H](Oc2cccc(C(F)(F)F)n2)[C@@H](O)[C@H]1O] | +++ |
| A333 | 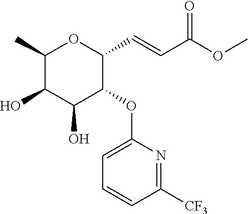 [SMILES: COC(=O)/C=C/[C@H]1O[C@H](C)[C@H](O)[C@H](O)[C@H]1Oc1cccc(C(F)(F)F)n1] | +++ |
| A334 | 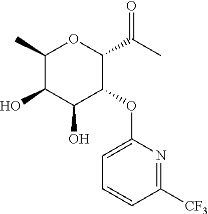 [SMILES: CC(=O)[C@H]1O[C@H](C)[C@H](O)[C@H](O)[C@H]1Oc1cccc(C(F)(F)F)n1] | +++ |
| A335 | 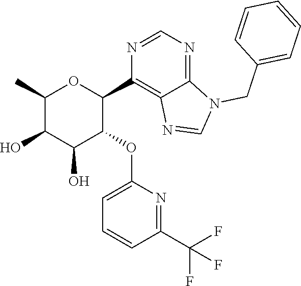 [SMILES: C[C@H]1O[C@@H](c2ncnc3c2ncn3Cc2ccccc2)[C@H](Oc2cccc(C(F)(F)F)n2)[C@@H](O)[C@H]1O] | +++ |
| A336 | 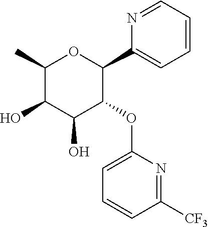 [SMILES: C[C@H]1O[C@@H](c2ccccn2)[C@H](Oc2cccc(C(F)(F)F)n2)[C@@H](O)[C@H]1O] | +++ |
| A337 | 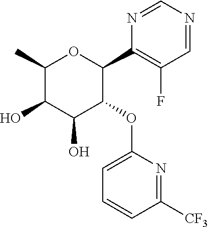 [SMILES: C[C@H]1O[C@@H](c2ncncc2F)[C@H](Oc2cccc(C(F)(F)F)n2)[C@@H](O)[C@H]1O] | +++ |
| A338 | 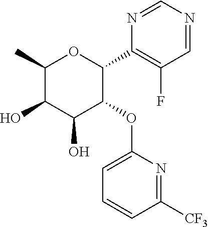 [SMILES: C[C@H]1O[C@H](c2ncncc2F)[C@H](Oc2cccc(C(F)(F)F)n2)[C@@H](O)[C@H]1O] | +++ |
| A339 | 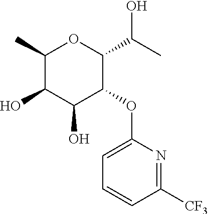 [SMILES: CC(O)[C@H]1O[C@H](C)[C@H](O)[C@H](O)[C@H]1Oc1cccc(C(F)(F)F)n1] | +++ |
| A340 | 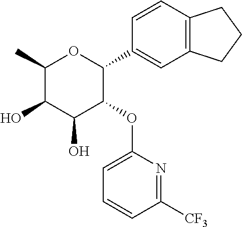 [SMILES: C[C@H]1O[C@H](c2ccc3c(c2)CCC3)[C@H](Oc2cccc(C(F)(F)F)n2)[C@@H](O)[C@H]1O] | +++ |
| A341 | 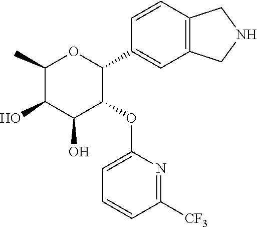 [SMILES: C[C@H]1O[C@H](c2ccc3c(c2)CNC3)[C@H](Oc2cccc(C(F)(F)F)n2)[C@@H](O)[C@H]1O] | +++ |
| A342 | 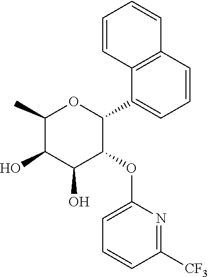 [SMILES: C[C@H]1O[C@H](c2cccc3ccccc23)[C@H](Oc2cccc(C(F)(F)F)n2)[C@@H](O)[C@H]1O] | +++ |
| A343 | 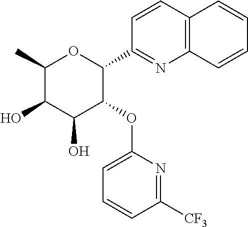 [SMILES: C[C@H]1O[C@H](c2ccc3ccccc3n2)[C@H](Oc2cccc(C(F)(F)F)n2)[C@@H](O)[C@H]1O] | +++ |
| A344 | 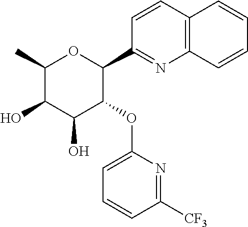 [SMILES: C[C@H]1O[C@@H](c2ccc3ccccc3n2)[C@H](Oc2cccc(C(F)(F)F)n2)[C@@H](O)[C@H]1O] | +++ |
| A345 | 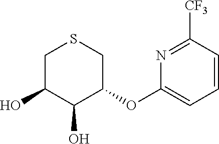 [SMILES: O[C@@H]1[C@@H](Oc2cccc(C(F)(F)F)n2)CSC[C@@H]1O] | +++ |

In the table above, K_{d} values that are >=100 μM=+, <100 μM and >10 μM=++ and <10 μM=+++

TABLE 1D
ASGPR Binding of Select Compounds

| | | ASGPR |
|---|---|---|
| Compound ID | Structure | K_{D} (μM) |
| A346 | 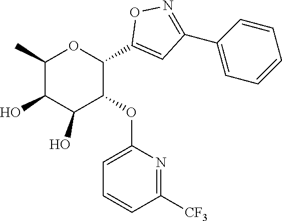 [SMILES: C[C@H]1O[C@H](c2cc(-c3ccccc3)no2)[C@H](Oc2cccc(C(F)(F)F)n2)[C@@H](O)[C@H]1O] | +++ |
| A347 | 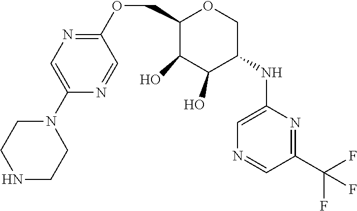 [SMILES: O[C@@H]1[C@H](O)[C@@H](Nc2cncc(C(F)(F)F)n2)CO[C@@H]1COc1cnc(N2CCNCC2)cn1] | |
| A348 | 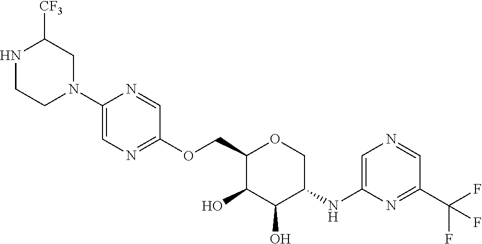 [SMILES: O[C@@H]1[C@H](O)[C@@H](Nc2cncc(C(F)(F)F)n2)CO[C@@H]1COc1cnc(N2CCNC(C(F)(F)F)C2)cn1] | |
| A349 | 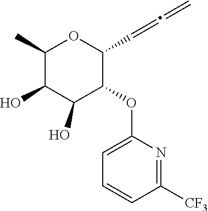 [SMILES: C=C=C[C@H]1O[C@H](C)[C@H](O)[C@H](O)[C@H]1Oc1cccc(C(F)(F)F)n1] | |
| A350 | 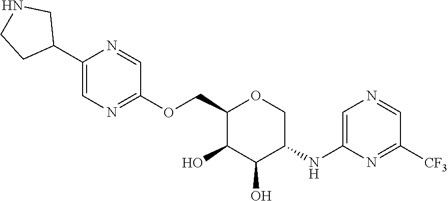 [SMILES: O[C@@H]1[C@H](O)[C@@H](Nc2cncc(C(F)(F)F)n2)CO[C@@H]1COc1cnc(C2CCNC2)cn1] | |
| A351 | 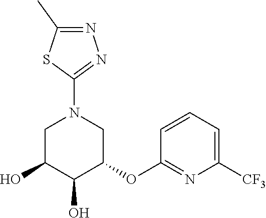 [SMILES: Cc1nnc(N2C[C@H](Oc3cccc(C(F)(F)F)n3)[C@@H](O)[C@@H](O)C2)s1] | +++ |
| A352 | 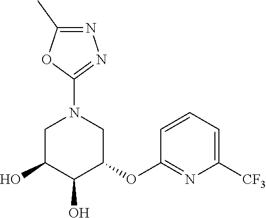 [SMILES: Cc1nnc(N2C[C@H](Oc3cccc(C(F)(F)F)n3)[C@@H](O)[C@@H](O)C2)o1] | +++ |
| A353 | 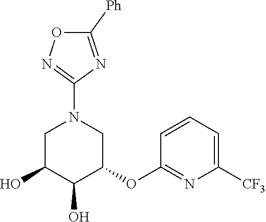 [SMILES: O[C@@H]1[C@@H](Oc2cccc(C(F)(F)F)n2)CN(c2noc(-c3ccccc3)n2)C[C@@H]1O] | +++ |
| A354 | 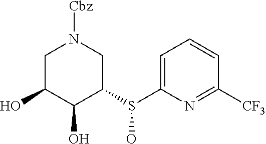 | ++ |
| A355 | 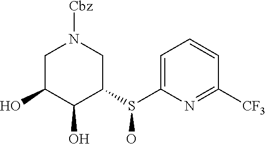 | >30 |
| A356 | 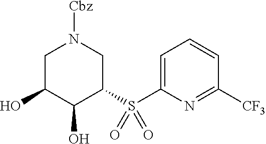 [SMILES: O=C(OCc1ccccc1)N1C[C@H](O)[C@H](O)[C@@H](S(=O)(=O)c2cccc(C(F)(F)F)n2)C1] | >30 |
| A357 | 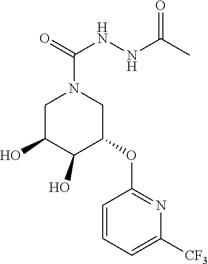 [SMILES: CC(=O)NNC(=O)N1C[C@H](Oc2cccc(C(F)(F)F)n2)[C@@H](O)[C@@H](O)C1] | +++ |
| A358 | 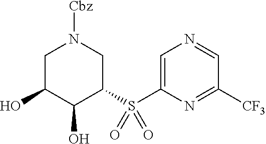 [SMILES: O=C(OCc1ccccc1)N1C[C@H](O)[C@H](O)[C@@H](S(=O)(=O)c2cncc(C(F)(F)F)n2)C1] | >30 |
| A359 | 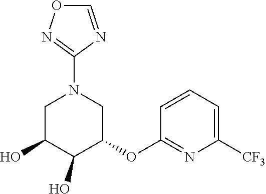 [SMILES: O[C@@H]1[C@@H](Oc2cccc(C(F)(F)F)n2)CN(c2ncon2)C[C@@H]1O] | +++ |
| A360 | 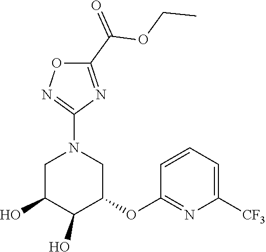 [SMILES: CCOC(=O)c1nc(N2C[C@H](Oc3cccc(C(F)(F)F)n3)[C@@H](O)[C@@H](O)C2)no1] | +++ |
| A361 | 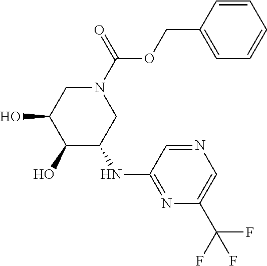 [SMILES: O=C(OCc1ccccc1)N1C[C@H](Nc2cncc(C(F)(F)F)n2)[C@@H](O)[C@@H](O)C1] | +++ |
| A362 | 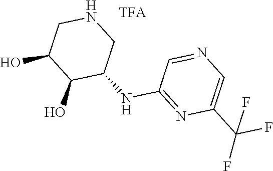 [SMILES: O[C@@H]1[C@@H](Nc2cncc(C(F)(F)F)n2)CNC[C@@H]1O] | +++ |
| A363 | 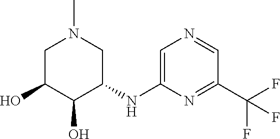 [SMILES: CN1C[C@H](Nc2cncc(C(F)(F)F)n2)[C@@H](O)[C@@H](O)C1] | +++ |
| A364 | 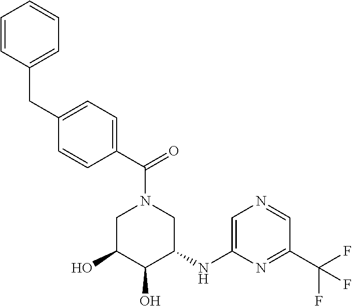 [SMILES: O=C(c1ccc(Cc2ccccc2)cc1)N1C[C@H](Nc2cncc(C(F)(F)F)n2)[C@@H](O)[C@@H](O)C1] | +++ |
| A365 | 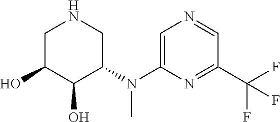 [SMILES: CN(c1cncc(C(F)(F)F)n1)[C@H]1CNC[C@H](O)[C@@H]1O] | +++ |
| A366 | 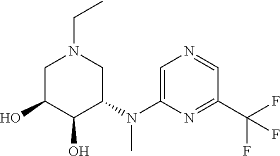 [SMILES: CCN1C[C@H](O)[C@H](O)[C@@H](N(C)c2cncc(C(F)(F)F)n2)C1] | +++ |
| A367 | 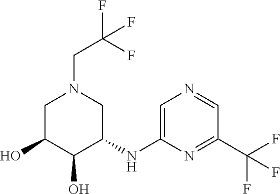 [SMILES: O[C@@H]1[C@@H](Nc2cncc(C(F)(F)F)n2)CN(CC(F)(F)F)C[C@@H]1O] | +++ |
| A368 | 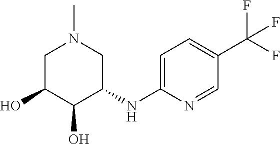 [SMILES: CN1C[C@H](Nc2ccc(C(F)(F)F)cn2)[C@@H](O)[C@@H](O)C1] | >30 |
| A369 | 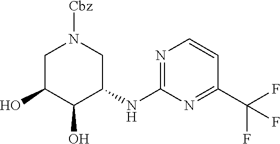 [SMILES: O=C(OCc1ccccc1)N1C[C@H](Nc2nccc(C(F)(F)F)n2)[C@@H](O)[C@@H](O)C1] | +++ |
| A370 | 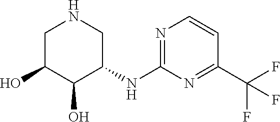 [SMILES: O[C@@H]1[C@@H](Nc2nccc(C(F)(F)F)n2)CNC[C@@H]1O] | ++ |
| A371 | 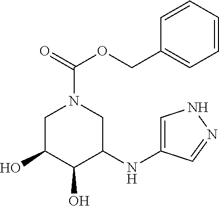 [SMILES: O=C(OCc1ccccc1)N1CC(Nc2cn[nH]c2)[C@@H](O)[C@@H](O)C1] | >30 |
| A372 | 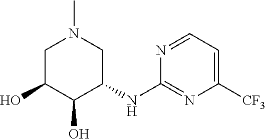 [SMILES: CN1C[C@H](Nc2nccc(C(F)(F)F)n2)[C@@H](O)[C@@H](O)C1] | +++ |
| A373 | 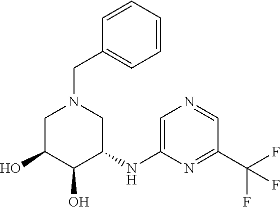 [SMILES: O[C@@H]1[C@@H](Nc2cncc(C(F)(F)F)n2)CN(Cc2ccccc2)C[C@@H]1O] | +++ |
| A374 | 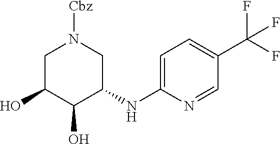 [SMILES: O=C(OCc1ccccc1)N1C[C@H](Nc2ccc(C(F)(F)F)cn2)[C@@H](O)[C@@H](O)C1] | >30 |
| A375 | 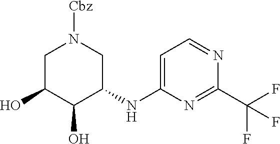 [SMILES: O=C(OCc1ccccc1)N1C[C@H](Nc2ccnc(C(F)(F)F)n2)[C@@H](O)[C@@H](O)C1] | +++ |
| A381 | 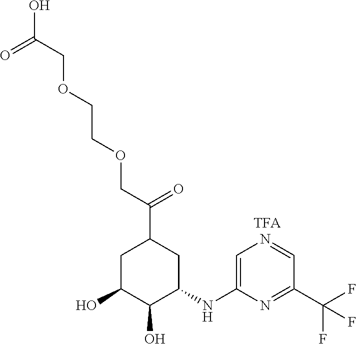 [SMILES: O=C(O)COCCOCC(=O)C1C[C@H](Nc2cncc(C(F)(F)F)n2)[C@@H](O)[C@@H](O)C1] | +++ |
| A380 | 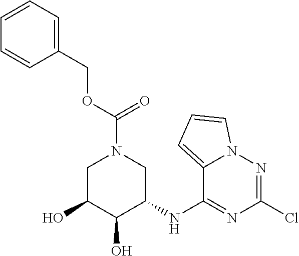 [SMILES: O=C(OCc1ccccc1)N1C[C@H](Nc2nc(Cl)nn3cccc23)[C@@H](O)[C@@H](O)C1] | +++ |
| A379 | 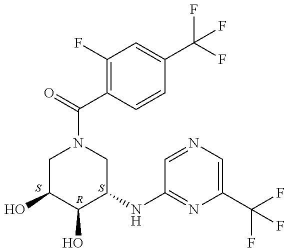 | +++ |
| A378 | 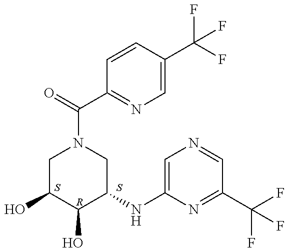 | +++ |
| A377 | 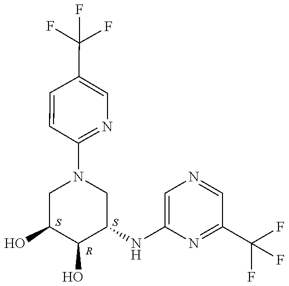 | +++ |
| A376 | 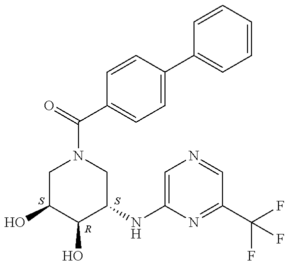 | +++ |
| A382 | 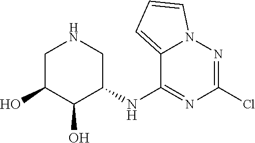 [SMILES: O[C@@H]1[C@@H](Nc2nc(Cl)nn3cccc23)CNC[C@@H]1O] | ++ |
| A383 | 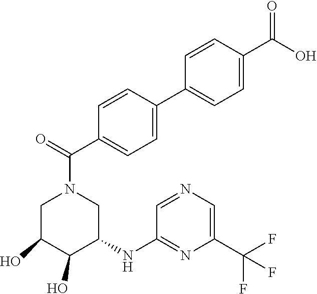 [SMILES: O=C(O)c1ccc(-c2ccc(C(=O)N3C[C@H](Nc4cncc(C(F)(F)F)n4)[C@@H](O)[C@@H](O)C3)cc2)cc1] | +++ |
| A384 | 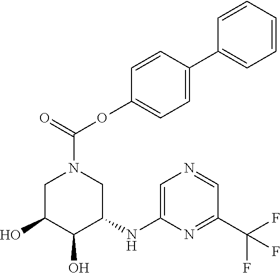 [SMILES: O=C(Oc1ccc(-c2ccccc2)cc1)N1C[C@H](Nc2cncc(C(F)(F)F)n2)[C@@H](O)[C@@H](O)C1] | +++ |
| A385 | 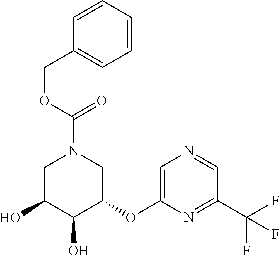 [SMILES: O=C(OCc1ccccc1)N1C[C@H](Oc2cncc(C(F)(F)F)n2)[C@@H](O)[C@@H](O)C1] | +++ |
| A386 | 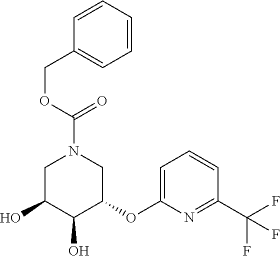 [SMILES: O=C(OCc1ccccc1)N1C[C@H](Oc2cccc(C(F)(F)F)n2)[C@@H](O)[C@@H](O)C1] | +++ |
| A387 | 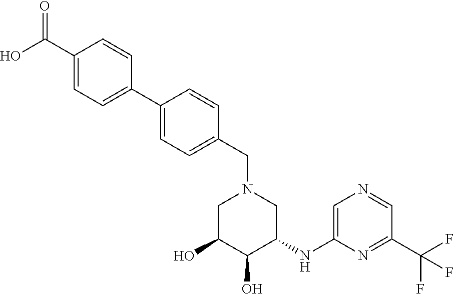 [SMILES: O=C(O)c1ccc(-c2ccc(CN3C[C@H](Nc4cncc(C(F)(F)F)n4)[C@@H](O)[C@@H](O)C3)cc2)cc1] | +++ |
| A388 | 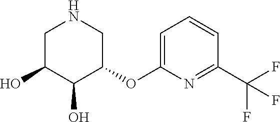 [SMILES: O[C@@H]1[C@@H](Oc2cccc(C(F)(F)F)n2)CNC[C@@H]1O] | +++ |
| A389 | 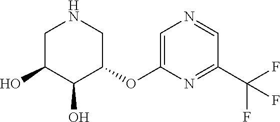 [SMILES: O[C@@H]1[C@@H](Oc2cncc(C(F)(F)F)n2)CNC[C@@H]1O] | +++ |
| A390 | 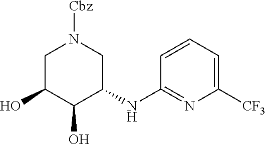 [SMILES: O=C(OCc1ccccc1)N1C[C@H](Nc2cccc(C(F)(F)F)n2)[C@@H](O)[C@@H](O)C1] | +++ |
| A391 | 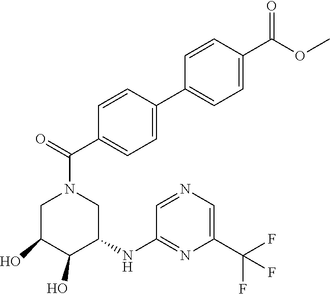 [SMILES: COC(=O)c1ccc(-c2ccc(C(=O)N3C[C@H](Nc4cncc(C(F)(F)F)n4)[C@@H](O)[C@@H](O)C3)cc2)cc1] | +++ |
| A392 | 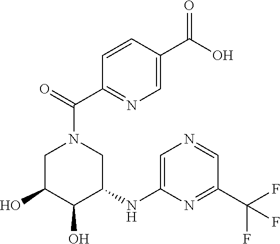 [SMILES: O=C(O)c1ccc(C(=O)N2C[C@H](Nc3cncc(C(F)(F)F)n3)[C@@H](O)[C@@H](O)C2)nc1] | +++ |
| A393 | 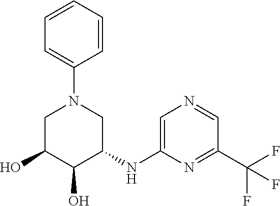 [SMILES: O[C@@H]1[C@@H](Nc2cncc(C(F)(F)F)n2)CN(c2ccccc2)C[C@@H]1O] | +++ |
| A394 | 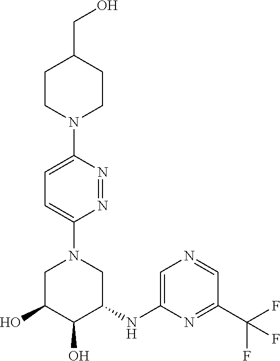 [SMILES: OCC1CCN(c2ccc(N3C[C@H](Nc4cncc(C(F)(F)F)n4)[C@@H](O)[C@@H](O)C3)nn2)CC1] | +++ |
| A395 | 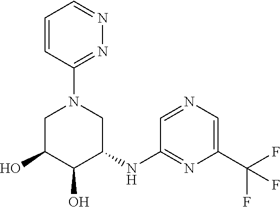 [SMILES: O[C@@H]1[C@@H](Nc2cncc(C(F)(F)F)n2)CN(c2cccnn2)C[C@@H]1O] | +++ |
| A396 | 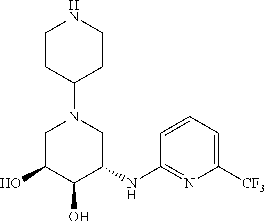 [SMILES: O[C@@H]1[C@@H](Nc2cccc(C(F)(F)F)n2)CN(C2CCNCC2)C[C@@H]1O] | +++ |
| A397 | 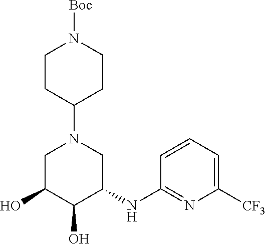 [SMILES: CC(C)(C)OC(=O)N1CCC(N2C[C@H](Nc3cccc(C(F)(F)F)n3)[C@@H](O)[C@@H](O)C2)CC1] | +++ |
| A398 | 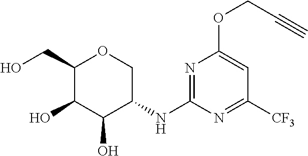 [SMILES: C#CCOc1cc(C(F)(F)F)nc(N[C@H]2CO[C@H](CO)[C@H](O)[C@@H]2O)n1] | |
| A399 | 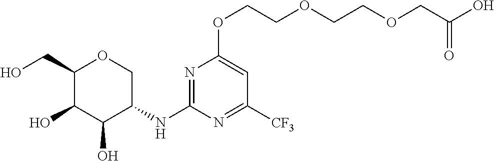 [SMILES: O=C(O)COCCOCCOc1cc(C(F)(F)F)nc(N[C@H]2CO[C@H](CO)[C@H](O)[C@@H]2O)n1] | |
| A400 | 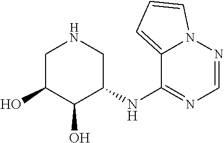 [SMILES: O[C@@H]1[C@@H](Nc2ncnn3cccc23)CNC[C@@H]1O] | >30 |
| A401 | 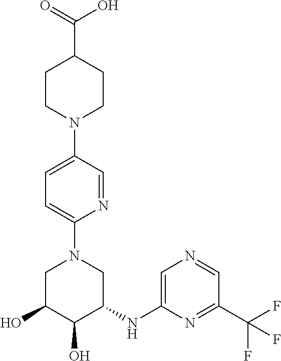 [SMILES: O=C(O)C1CCN(c2ccc(N3C[C@H](Nc4cncc(C(F)(F)F)n4)[C@@H](O)[C@@H](O)C3)nc2)CC1] | +++ |
| A402 | 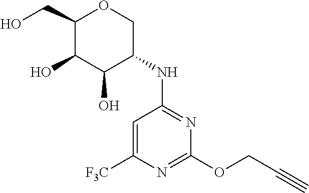 [SMILES: C#CCOc1nc(N[C@H]2CO[C@H](CO)[C@H](O)[C@@H]2O)cc(C(F)(F)F)n1] | ++ |
| A403 | 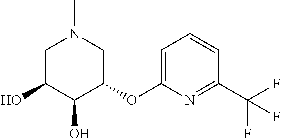 [SMILES: CN1C[C@H](Oc2cccc(C(F)(F)F)n2)[C@@H](O)[C@@H](O)C1] | ++ |
| A404 | 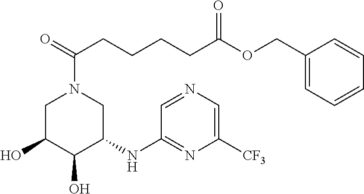 [SMILES: O=C(CCCCC(=O)N1C[C@H](Nc2cncc(C(F)(F)F)n2)[C@@H](O)[C@@H](O)C1)OCc1ccccc1] | +++ |
| A405 | 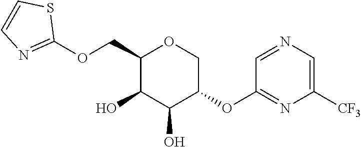 [SMILES: O[C@@H]1[C@H](O)[C@@H](Oc2cncc(C(F)(F)F)n2)CO[C@@H]1COc1nccs1] | |
| A406 | 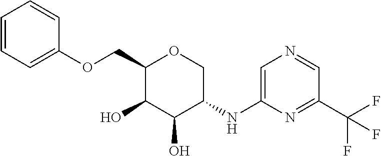 [SMILES: O[C@@H]1[C@H](O)[C@@H](Nc2cncc(C(F)(F)F)n2)CO[C@@H]1COc1ccccc1] | |
| A407 | 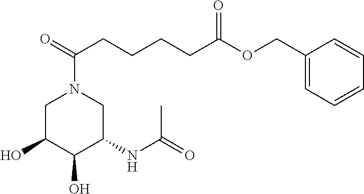 [SMILES: CC(=O)N[C@H]1CN(C(=O)CCCCC(=O)OCc2ccccc2)C[C@H](O)[C@@H]1O] | ++ |
| A408 | 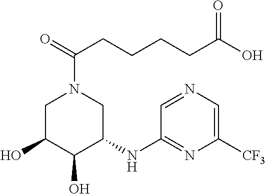 [SMILES: O=C(O)CCCCC(=O)N1C[C@H](Nc2cncc(C(F)(F)F)n2)[C@@H](O)[C@@H](O)C1] | +++ |
| A409 | 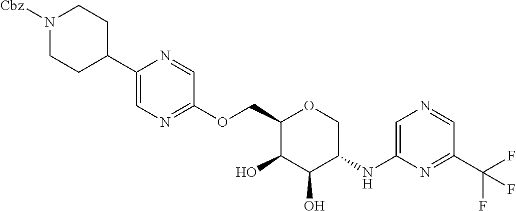 [SMILES: O=C(OCc1ccccc1)N1CCC(c2cnc(OC[C@H]3OC[C@H](Nc4cncc(C(F)(F)F)n4)[C@@H](O)[C@H]3O)cn2)CC1] | +++ |
| A410 | 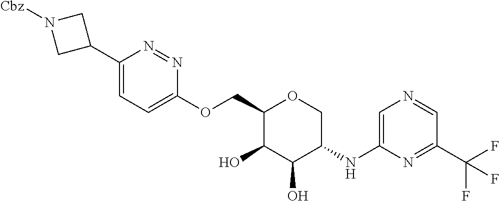 [SMILES: O=C(OCc1ccccc1)N1CC(c2ccc(OC[C@H]3OC[C@H](Nc4cncc(C(F)(F)F)n4)[C@@H](O)[C@H]3O)nn2)C1] | +++ |
| A412 | 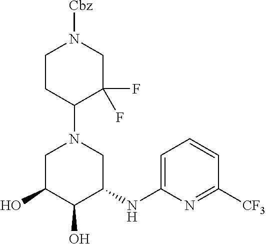 [SMILES: O=C(OCc1ccccc1)N1CCC(N2C[C@H](Nc3cccc(C(F)(F)F)n3)[C@@H](O)[C@@H](O)C2)C(F)(F)C1] | +++ |
| A415 | 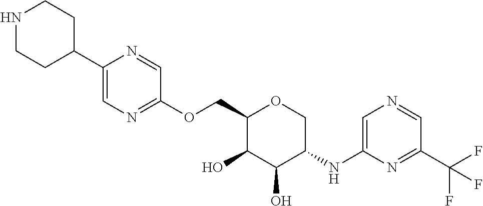 [SMILES: O[C@@H]1[C@H](O)[C@@H](Nc2cncc(C(F)(F)F)n2)CO[C@@H]1COc1cnc(C2CCNCC2)cn1] | +++ |
| A416 | 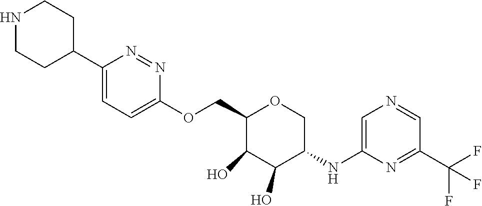 [SMILES: O[C@@H]1[C@H](O)[C@@H](Nc2cncc(C(F)(F)F)n2)CO[C@@H]1COc1ccc(C2CCNCC2)nn1] | +++ |
| A417 | 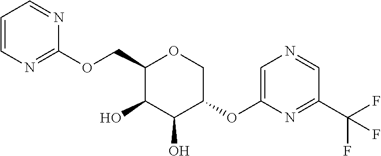 [SMILES: O[C@@H]1[C@H](O)[C@@H](Oc2cncc(C(F)(F)F)n2)CO[C@@H]1COc1ncccn1] | +++ |
| A418 | 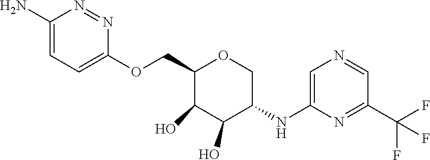 [SMILES: Nc1ccc(OC[C@H]2OC[C@H](Nc3cncc(C(F)(F)F)n3)[C@@H](O)[C@H]2O)nn1] | +++ |
| A419 | 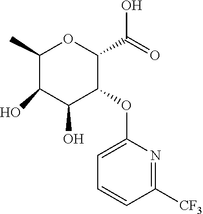 [SMILES: C[C@H]1O[C@H](C(=O)O)[C@H](Oc2cccc(C(F)(F)F)n2)[C@@H](O)[C@H]1O] | |
| A420 | 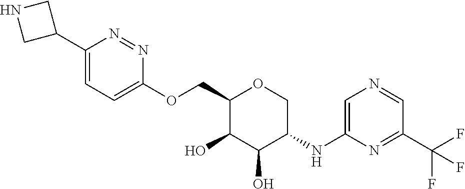 [SMILES: O[C@@H]1[C@H](O)[C@@H](Nc2cncc(C(F)(F)F)n2)CO[C@@H]1COc1ccc(C2CNC2)nn1] | +++ |
| A421 | 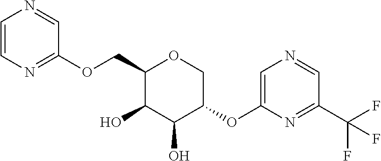 [SMILES: O[C@@H]1[C@H](O)[C@@H](Oc2cncc(C(F)(F)F)n2)CO[C@@H]1COc1cnccn1] | +++ |
| A424 | 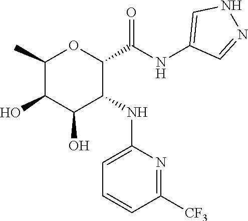 [SMILES: C[C@H]1O[C@H](C(=O)Nc2cn[nH]c2)[C@H](Nc2cccc(C(F)(F)F)n2)[C@@H](O)[C@H]1O] | +++ |
| A422 | 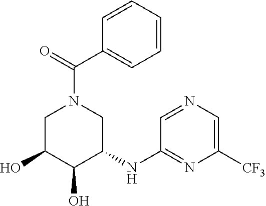 [SMILES: O=C(c1ccccc1)N1C[C@H](Nc2cncc(C(F)(F)F)n2)[C@@H](O)[C@@H](O)C1] | +++ |
| A423 | 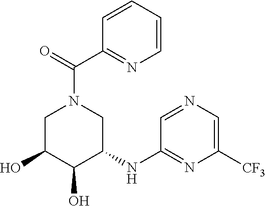 [SMILES: O=C(c1ccccn1)N1C[C@H](Nc2cncc(C(F)(F)F)n2)[C@@H](O)[C@@H](O)C1] | +++ |
| A425 | 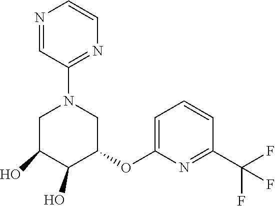 [SMILES: O[C@@H]1[C@@H](Oc2cccc(C(F)(F)F)n2)CN(c2cnccn2)C[C@@H]1O] | +++ |
| A426 | 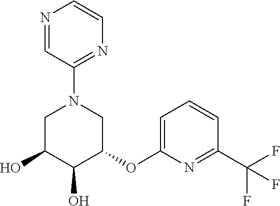 [SMILES: O[C@@H]1[C@@H](Oc2cccc(C(F)(F)F)n2)CN(c2cnccn2)C[C@@H]1O] | +++ |
| A427 | 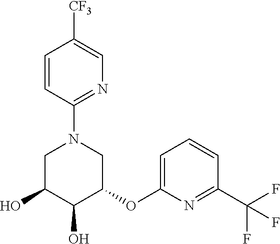 [SMILES: O[C@@H]1[C@@H](Oc2cccc(C(F)(F)F)n2)CN(c2ccc(C(F)(F)F)cn2)C[C@@H]1O] | +++ |
| A248 | 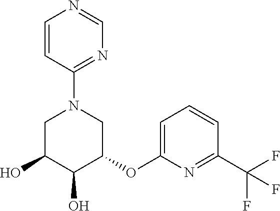 [SMILES: O[C@@H]1[C@@H](Oc2cccc(C(F)(F)F)n2)CN(c2ccncn2)C[C@@H]1O] | +++ |
| A429 | 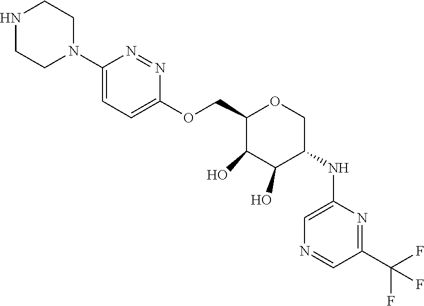 [SMILES: O[C@@H]1[C@H](O)[C@@H](Nc2cncc(C(F)(F)F)n2)CO[C@@H]1COc1ccc(N2CCNCC2)nn1] | |
| A430 | 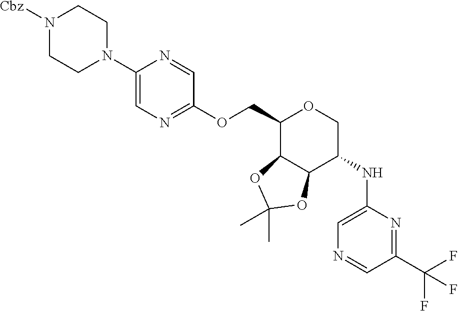 [SMILES: CC1(C)O[C@@H]2[C@H](O1)[C@@H](Nc1cncc(C(F)(F)F)n1)CO[C@@H]2COc1cnc(N2CCN(C(=O)OCc3ccccc3)CC2)cn1] | |

In the table above, K_{d} values that are >=100 μM=+, <100 μM and >10 μM=++ and <10 μM=+++

TABLE 1E
ASGPR Binding of Select Compounds

| Compound | | ASGPR |
|---|---|---|
| ID | Structure | K_{D} (μM) |
| A431 | 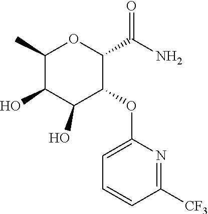 [SMILES: C[C@H]1O[C@H](C(N)=O)[C@H](Oc2cccc(C(F)(F)F)n2)[C@@H](O)[C@H]1O] | +++ |
| A432 | 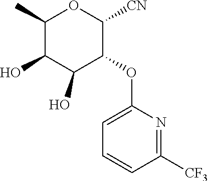 [SMILES: C[C@H]1O[C@H](C#N)[C@H](Oc2cccc(C(F)(F)F)n2)[C@@H](O)[C@H]1O] | +++ |
| A433 | 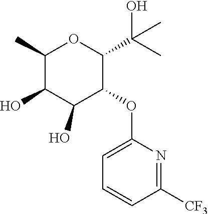 [SMILES: C[C@H]1O[C@H](C(C)(C)O)[C@H](Oc2cccc(C(F)(F)F)n2)[C@@H](O)[C@H]1O] | +++ |
| A434 | 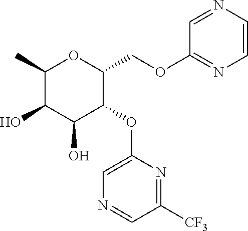 [SMILES: C[C@H]1O[C@H](COc2cnccn2)[C@H](Oc2cncc(C(F)(F)F)n2)[C@@H](O)[C@H]1O] | +++ |
| A435 | 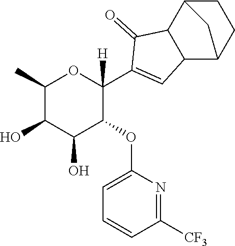 [SMILES: C[C@H]1O[C@H](C2=CC3C4CCC(C4)C3C2=O)[C@H](Oc2cccc(C(F)(F)F)n2)[C@@H](O)[C@H]1O] | +++ |
| A435A | 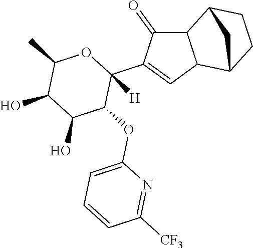 [SMILES: C[C@H]1O[C@H](C2=CC3C(C2=O)[C@@H]2CC[C@H]3C2)[C@H](Oc2cccc(C(F)(F)F)n2)[C@@H](O)[C@H]1O] | +++ |
| A435B | 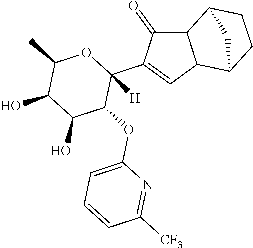 [SMILES: C[C@H]1O[C@H](C2=CC3C(C2=O)[C@H]2CC[C@@H]3C2)[C@H](Oc2cccc(C(F)(F)F)n2)[C@@H](O)[C@H]1O] | +++ |
| A436 | 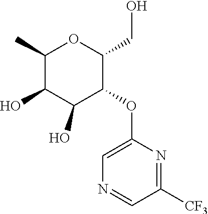 [SMILES: C[C@H]1O[C@H](CO)[C@H](Oc2cncc(C(F)(F)F)n2)[C@@H](O)[C@H]1O] | >30 |
| A437 | 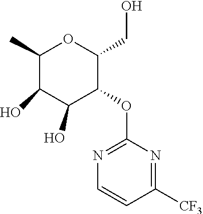 [SMILES: C[C@H]1O[C@H](CO)[C@H](Oc2nccc(C(F)(F)F)n2)[C@@H](O)[C@H]1O] | ++ |
| A438 | 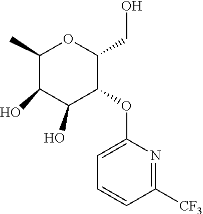 [SMILES: C[C@H]1O[C@H](CO)[C@H](Oc2cccc(C(F)(F)F)n2)[C@@H](O)[C@H]1O] | +++ |
| A439 | 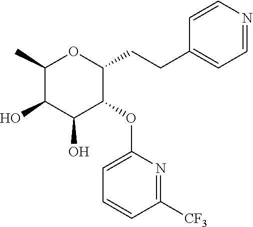 [SMILES: C[C@H]1O[C@H](CCc2ccncc2)[C@H](Oc2cccc(C(F)(F)F)n2)[C@@H](O)[C@H]1O] | +++ |
| A440 | 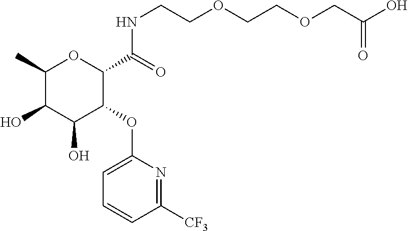 [SMILES: C[C@H]1O[C@H](C(=O)NCCOCCOCC(=O)O)[C@H](Oc2cccc(C(F)(F)F)n2)[C@@H](O)[C@H]1O] | >30 |
| A441 | 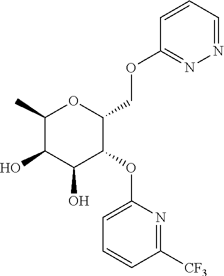 [SMILES: C[C@H]1O[C@H](COc2cccnn2)[C@H](Oc2cccc(C(F)(F)F)n2)[C@@H](O)[C@H]1O] | +++ |
| A442 | 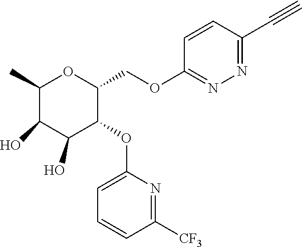 [SMILES: C#Cc1ccc(OC[C@H]2O[C@H](C)[C@H](O)[C@H](O)[C@H]2Oc2cccc(C(F)(F)F)n2)nn1] | +++ |
| A443 | 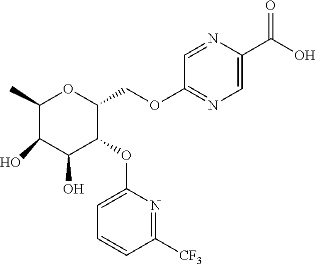 [SMILES: C[C@H]1O[C@H](COc2cnc(C(=O)O)cn2)[C@H](Oc2cccc(C(F)(F)F)n2)[C@@H](O)[C@H]1O] | +++ |
| A444 | 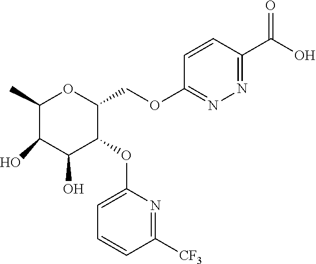 [SMILES: C[C@H]1O[C@H](COc2ccc(C(=O)O)nn2)[C@H](Oc2cccc(C(F)(F)F)n2)[C@@H](O)[C@H]1O] | +++ |
| A445 | 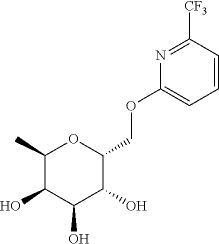 [SMILES: C[C@H]1O[C@H](COc2cccc(C(F)(F)F)n2)[C@H](O)[C@@H](O)[C@H]1O] | ++ |
| A446 | 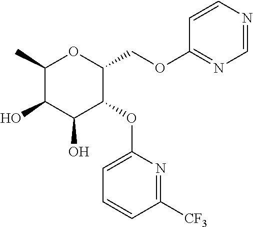 [SMILES: C[C@H]1O[C@H](COc2ccncn2)[C@H](Oc2cccc(C(F)(F)F)n2)[C@@H](O)[C@H]1O] | +++ |
| A447 | 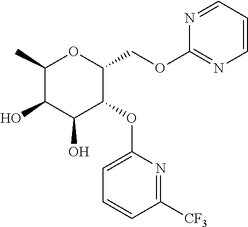 [SMILES: C[C@H]1O[C@H](COc2ncccn2)[C@H](Oc2cccc(C(F)(F)F)n2)[C@@H](O)[C@H]1O] | +++ |
| A448 | 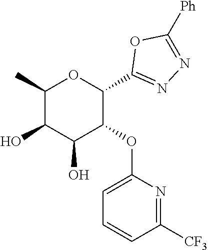 [SMILES: C[C@H]1O[C@H](c2nnc(-c3ccccc3)o2)[C@H](Oc2cccc(C(F)(F)F)n2)[C@@H](O)[C@H]1O] | +++ |
| A449 | 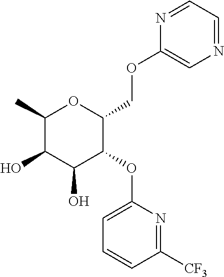 [SMILES: C[C@H]1O[C@H](COc2cnccn2)[C@H](Oc2cccc(C(F)(F)F)n2)[C@@H](O)[C@H]1O] | +++ |
| A450 | 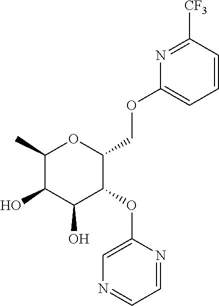 [SMILES: C[C@H]1O[C@H](COc2cccc(C(F)(F)F)n2)[C@H](Oc2cnccn2)[C@@H](O)[C@H]1O] | +++ |
| A451 | 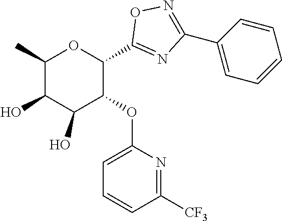 [SMILES: C[C@H]1O[C@H](c2nc(-c3ccccc3)no2)[C@H](Oc2cccc(C(F)(F)F)n2)[C@@H](O)[C@H]1O] | +++ |
| A452 | 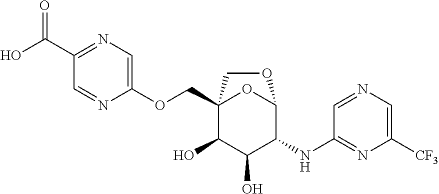 [SMILES: O=C(O)c1cnc(OC[C@@]23CO[C@@H](O2)[C@H](Nc2cncc(C(F)(F)F)n2)[C@@H](O)[C@H]3O)cn1] | +++ |
| A453 | 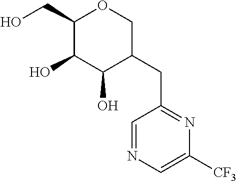 [SMILES: OC[C@H]1OCC(Cc2cncc(C(F)(F)F)n2)[C@@H](O)[C@H]1O] | |
| A454 | 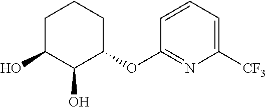 [SMILES: O[C@@H]1[C@@H](Oc2cccc(C(F)(F)F)n2)CCC[C@@H]1O] | |

In the table above, K_{d} values that are >=100 μM=+, 100 μM and >10 μM=++ and <10 μM=+++

### Example 2 TNF Cellular Assays

### TNF FP Assay

TNF (20 nM) and test compound in 47 mM HEPES pH 6.5, 47 mM NaCl, 0.9 mM EDTA, 0.007% Triton X-100 were incubated at 25° C. for 2 hours. Fluorescence data was read with an Envision and plotted versus Compound concentration and fit to a single-site binding model.

### TNF Ternary Complex Assay

His-tagged TNF (300 nM), biotinylated ASGPR (25 nM) and test compound in 50 mM Tris, pH 7.5, 150 mM NaCl, 0.01% Tween-20 are incubated at 25° C. for 4 hours. Streptavidin-Tb cryptate (PerkinElmer 610SATLA) and anti-His-d2 (Perkin Elmer 61HISDLA) are then added and incubated for 2 hour or overnight. Fluorescence data is read with an Envision and the ratio of the 655 nm/615 nm signals plotted versus test compound concentration.

### TNF Degradation by Test Compound in HepG2 Cells

Test compound and human TNF were pre-incubated in phosphate-buffered saline, pH 7.4 (ThermoFisher 10010023) for 18 hours to allow formation of binary complexes. Compound/TNF complexes or TNF alone were incubated for 2 hours on HepG2 cells grown in Dulbecco's Modified Eagle Medium for 16-24 hours to near confluence. Cells were washed with Dulbecco's Modified Eagle Medium, 10% fetal bovine serum. Cells were then incubated with cell media without reagents for 0-3 hours. Cells were washed and lysed for Western blot analysis using phosphate-buffered saline containing RIPA (Sigma R⁰²⁷⁸) and protease inhibitors (ThermoScientific 1861279). Samples were run on 4-20% o gradient SDS-PAGE gel (Biorad 5678094) and transferred to PVDF and blocked for 1 hour with LI-COR blocking solution. TNF and actin were detected with anti-IgG and anti-actin antibodies, respectively, and 800cw-conjugated secondary antibody and read with a LI-COR. Test compound and human TNF were pre-incubated for 18 h to allow formation of binary complexes. Test compound TNF complexes or TNF alone were incubated for 2 h on HepG2 cells. Cells were washed, incubated with cell media without reagents for 0-24 h and subsequently washed and lysed for Western blot analysis. TNF and actin were detected with anti-IgG and anti-actin antibodies, respectively, and 800cw-conjugated secondary antibody.

TABLE 2
ASGPR Binding and TNF Degradation Activity of Select Compounds

| | | ASGPR | TNF |
|---|---|---|---|
| | | SPR | FP |
| | | Bind- | As- |
| | | ing | say |
| Cmpd | | K_{D} | IC₅₀ |
| # | Structure | (nM) | (nM) |
| 1 | 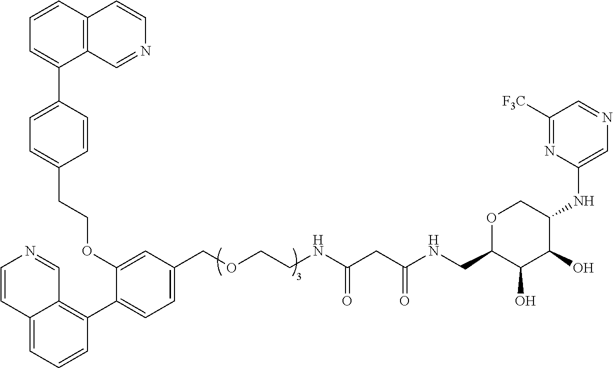 | **** | *** |
| 2 | 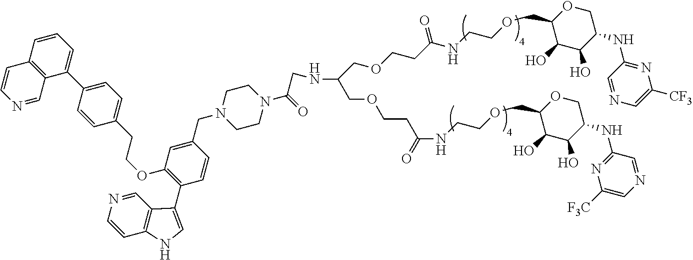 | **** | * |
| 3 | 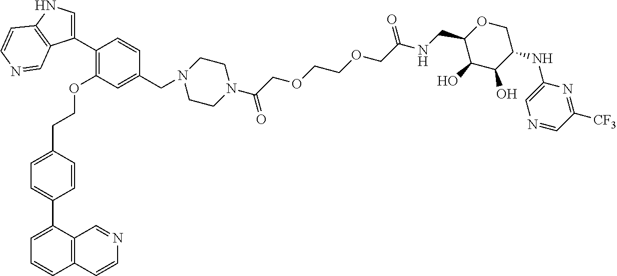 [SMILES: O=C(COCCOCC(=O)N1CCN(Cc2ccc(-c3c[nH]c4ccncc34)c(OCCc3ccc(-c4cccc5ccncc45)cc3)c2)CC1)NC[C@H]1OC[C@H](Nc2cncc(C(F)(F)F)n2)[C@@H](O)[C@H]1O] | **** | **** |
| 4 | 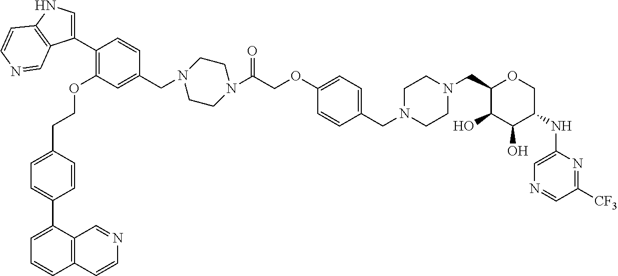 [SMILES: O=C(COc1ccc(CN2CCN(C[C@H]3OC[C@H](Nc4cncc(C(F)(F)F)n4)[C@@H](O)[C@H]3O)CC2)cc1)N1CCN(Cc2ccc(-c3c[nH]c4ccncc34)c(OCCc3ccc(-c4cccc5ccncc45)cc3)c2)CC1] | **** | >112 |
| 5 | 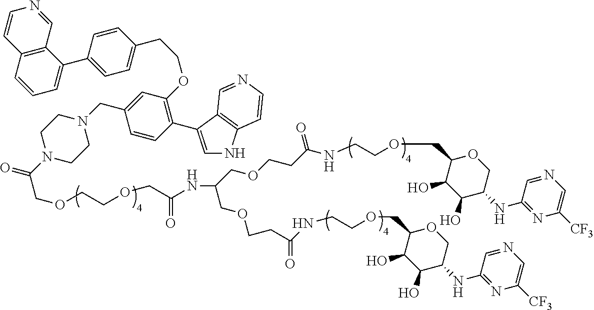 | >25 | >136 |
| 6 | 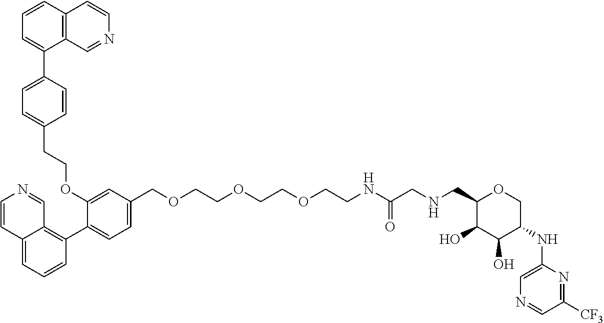 [SMILES: O=C(CNC[C@H]1OC[C@H](Nc2cncc(C(F)(F)F)n2)[C@@H](O)[C@H]1O)NCCOCCOCCOCc1ccc(-c2cccc3ccncc23)c(OCCc2ccc(-c3cccc4ccncc34)cc2)c1] | | *** |
| 7 | 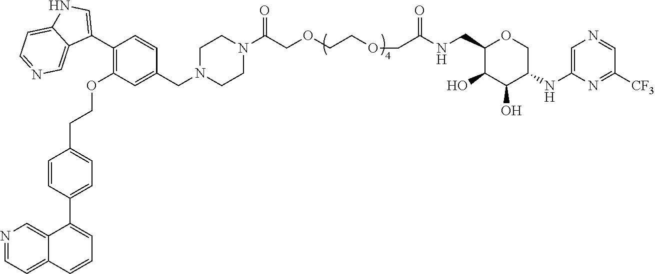 | **** | **** |
| 8 | 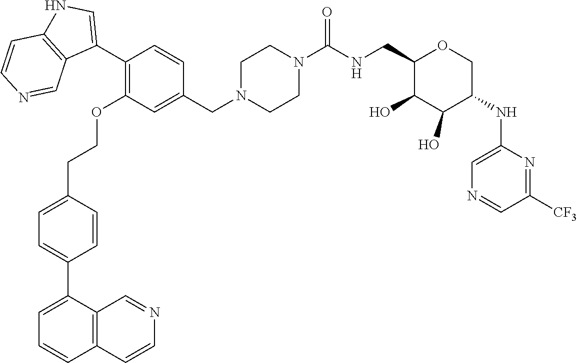 [SMILES: O=C(NC[C@H]1OC[C@H](Nc2cncc(C(F)(F)F)n2)[C@@H](O)[C@H]1O)N1CCN(Cc2ccc(-c3c[nH]c4ccncc34)c(OCCc3ccc(-c4cccc5ccncc45)cc3)c2)CC1] | **** | * |
| 9 | 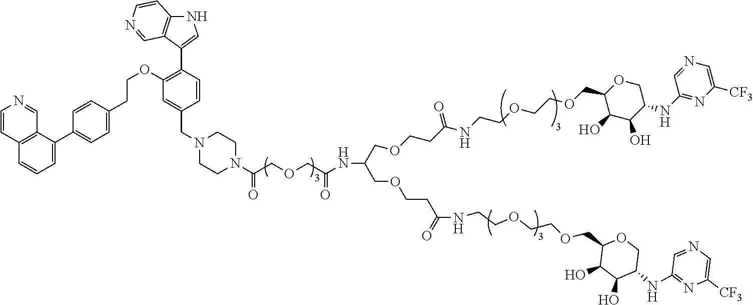 | **** | * |
| 10 | 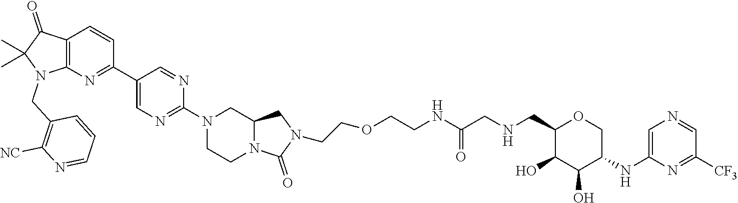 [SMILES: CC1(C)C(=O)c2ccc(-c3cnc(N4CCN5C(=O)N(CCOCCNC(=O)CNC[C@H]6OC[C@H](Nc7cncc(C(F)(F)F)n7)[C@@H](O)[C@H]6O)C[C@@H]5C4)nc3)nc2N1Cc1cccnc1C#N] | **** | * |
| 11 | 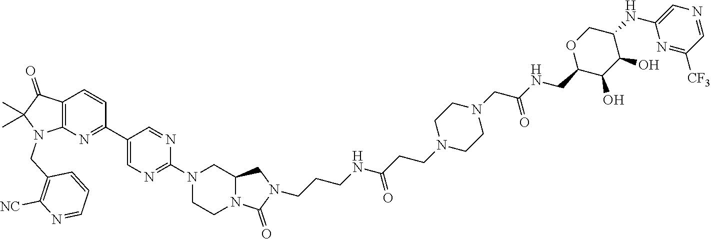 [SMILES: CC1(C)C(=O)c2ccc(-c3cnc(N4CCN5C(=O)N(CCCNC(=O)CCN6CCN(CC(=O)NC[C@H]7OC[C@H](Nc8cncc(C(F)(F)F)n8)[C@@H](O)[C@H]7O)CC6)C[C@@H]5C4)nc3)nc2N1Cc1cccnc1C#N] | **** | *** |
| 12 | 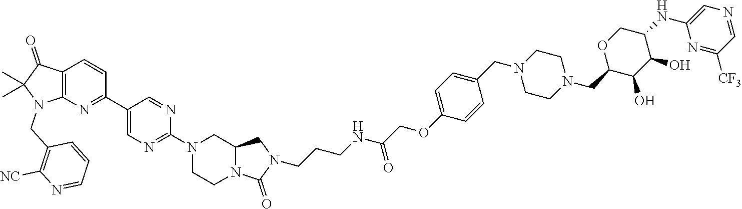 [SMILES: CC1(C)C(=O)c2ccc(-c3cnc(N4CCN5C(=O)N(CCCNC(=O)COc6ccc(CN7CCN(C[C@H]8OC[C@H](Nc9cncc(C(F)(F)F)n9)[C@@H](O)[C@H]8O)CC7)cc6)C[C@@H]5C4)nc3)nc2N1Cc1cccnc1C#N] | **** | *** |
| 13 | 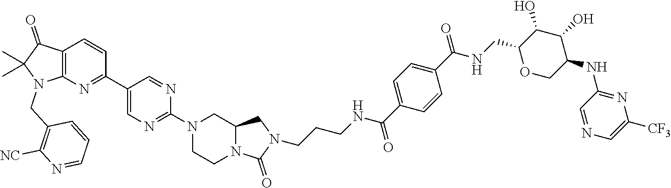 [SMILES: CC1(C)C(=O)c2ccc(-c3cnc(N4CCN5C(=O)N(CCCNC(=O)c6ccc(C(=O)NC[C@H]7OC[C@H](Nc8cncc(C(F)(F)F)n8)[C@@H](O)[C@H]7O)cc6)C[C@@H]5C4)nc3)nc2N1Cc1cccnc1C#N] | **** | * |
| 14 | 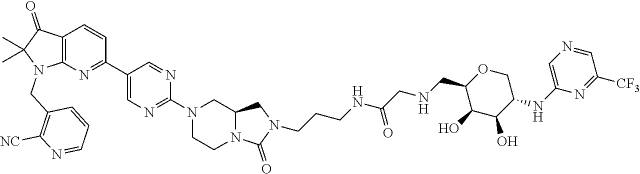 [SMILES: CC1(C)C(=O)c2ccc(-c3cnc(N4CCN5C(=O)N(CCCNC(=O)CNC[C@H]6OC[C@H](Nc7cncc(C(F)(F)F)n7)[C@@H](O)[C@H]6O)C[C@@H]5C4)nc3)nc2N1Cc1cccnc1C#N] | **** | *** |
| 15 | 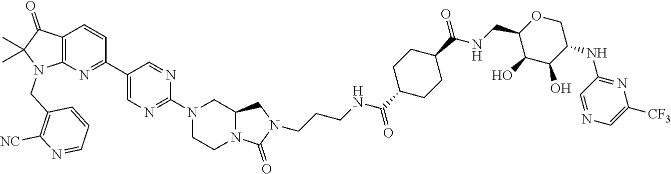 [SMILES: CC1(C)C(=O)c2ccc(-c3cnc(N4CCN5C(=O)N(CCCNC(=O)[C@H]6CC[C@H](C(=O)NC[C@H]7OC[C@H](Nc8cncc(C(F)(F)F)n8)[C@@H](O)[C@H]7O)CC6)C[C@@H]5C4)nc3)nc2N1Cc1cccnc1C#N] | **** | * |
| 16 | 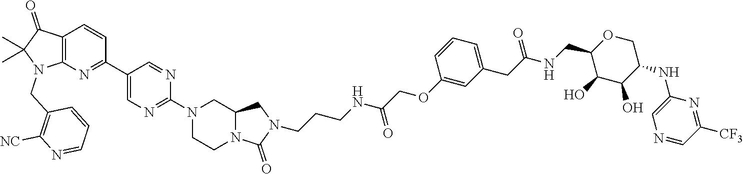 [SMILES: CC1(C)C(=O)c2ccc(-c3cnc(N4CCN5C(=O)N(CCCNC(=O)COc6cccc(CC(=O)NC[C@H]7OC[C@H](Nc8cncc(C(F)(F)F)n8)[C@@H](O)[C@H]7O)c6)C[C@@H]5C4)nc3)nc2N1Cc1cccnc1C#N] | **** | * |
| 17 | 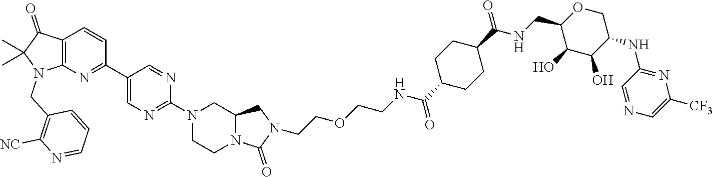 [SMILES: CC1(C)C(=O)c2ccc(-c3cnc(N4CCN5C(=O)N(CCOCCNC(=O)[C@H]6CC[C@H](C(=O)NC[C@H]7OC[C@H](Nc8cncc(C(F)(F)F)n8)[C@@H](O)[C@H]7O)CC6)C[C@@H]5C4)nc3)nc2N1Cc1cccnc1C#N] | **** | ** |
| 18 | 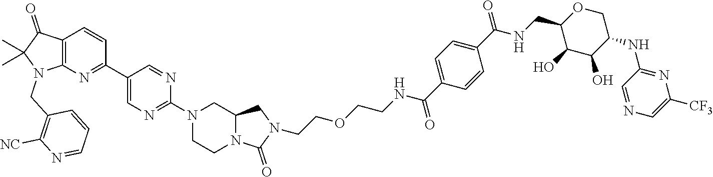 [SMILES: CC1(C)C(=O)c2ccc(-c3cnc(N4CCN5C(=O)N(CCOCCNC(=O)c6ccc(C(=O)NC[C@H]7OC[C@H](Nc8cncc(C(F)(F)F)n8)[C@@H](O)[C@H]7O)cc6)C[C@@H]5C4)nc3)nc2N1Cc1cccnc1C#N] | | * |
| 19 | 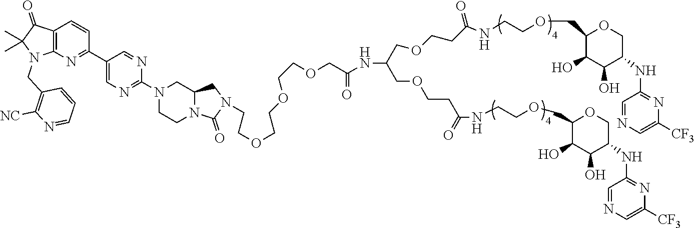 | **** | |
| 20 | 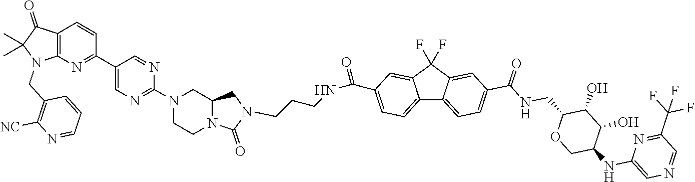 [SMILES: CC1(C)C(=O)c2ccc(-c3cnc(N4CCN5C(=O)N(CCCNC(=O)c6ccc7c(c6)C(F)(F)c6cc(C(=O)NC[C@H]8OC[C@H](Nc9cncc(C(F)(F)F)n9)[C@@H](O)[C@H]8O)ccc6-7)C[C@@H]5C4)nc3)nc2N1Cc1cccnc1C#N] | **** | |
| 21 | 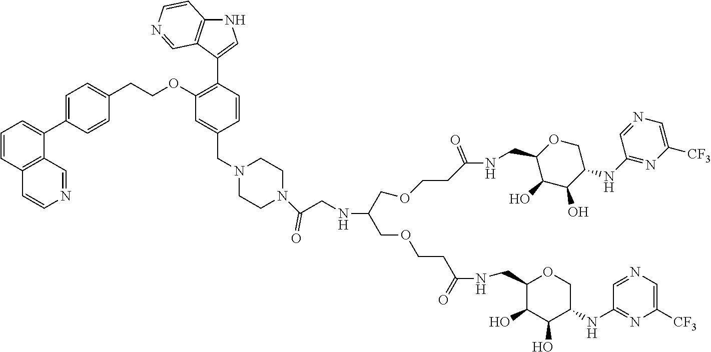 [SMILES: O=C(CCOCC(COCCC(=O)NC[C@H]1OC[C@H](Nc2cncc(C(F)(F)F)n2)[C@@H](O)[C@H]1O)NCC(=O)N1CCN(Cc2ccc(-c3c[nH]c4ccncc34)c(OCCc3ccc(-c4cccc5ccncc45)cc3)c2)CC1)NC[C@H]1OC[C@H](Nc2cncc(C(F)(F)F)n2)[C@@H](O)[C@H]1O] | **** | |
| 22 | 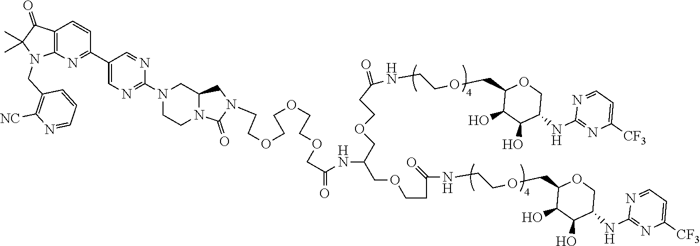 | **** | |
| 23 | 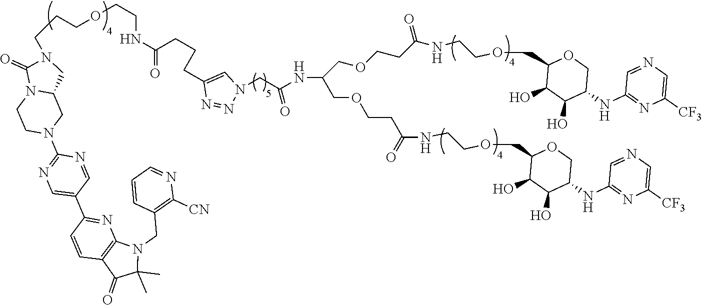 | | |

In the table above, Kd and IC50 values that are >1000 nM=*, 1000-500 nM=**, 500-100 nM=***, and <100=****

### Example 3. Binding of IgG Degrading Compounds to IgG and ASGPR, Ternary Complex Formation, and HepG2 Cellular Uptake

### Ternary Complex Formation Assay

The ternary complex formation assay is designed to identify the concentration of Degrader required to form a complex between the degrader, a constant concentration of fluorescently-labeled antibody and the ASGPR receptor on the cell surface. The peak mean fluorescence intensity (MFI) occurs at the optimal degrader concentration for ternary complex formation. Sub-optimal concentrations of degrader (too much or too little) results in fewer complexes formed and a reduced MFI.

Human IgG (Sigma-Aldrich, 14506) was covalently labeled with Alexa Fluor 488 using the Alexa Fluor™ 488 Protein Labeling Kit (Thermo Fisher, A10235) following the manufacturer's instructions. HepG2 cells (ATCC, HB-8065) were cultured to about 70% confluence and harvested by trypsinization. Single cell suspensions of HepG2 cells were plated in 96 well plates at 1×105 cells/well in cell culture media (DMEM supplemented with 10% FBS) and placed at 4° C. For studies using human IgG, cells were incubated with 100 nM AF488-labeled anti-human IgG and serial dilutions of IgG-based degrader for 1 hour at 4° C. For studies using DNP, cells were incubated with 100 nM AF488-labeled polyclonal rabbit anti-dinitrophenyl (DNP) antibodies (ThermoFisher) and serial dilutions of DNP-based degrader for 1 hour at 4° C. After washing once with ice-cold PBS containing 0.1% BSA and 0.1% sodium azide, cells were resuspended in 150 uL PBS+0.1% BSA and cell-associated AF488 fluorescence was measured by flow cytometry using the iQue® Screener PLUS Flow Cytometer (IntelliCyt). Data shows measured MFI.

### Cellular Uptake Assay

The cellular uptake assay is designed to measure compound-mediated cellular uptake of fluorescently labeled antibody following ternary complex formation with the ASGPR receptor and endocytosis of the entire complex into cells. The optimal concentration of compound will result in the maximum number of cells with intracellular antibody and is represented as the peak ratio of antibody-positive cells over the total number of cells. As with the ternary complex formation assay, too much or too little compound will result in suboptimal complex formation and fewer cells will have measurable intracellular fluorescence.

Human IgG (Sigma-Aldrich, 14506) was covalently labeled with Alexa Fluor 488 using the Alexa Fluor™ 488 Protein Labeling Kit (Thermo Fisher, A10235) following the manufacturer's instructions. HepG2 cells were plated in DMEM supplemented with 10% FBS at 5,000 cells per well in a 96-well microtiter plate and incubated for approximately 18 hours at 37° C./5% CO2. For studies using human IgG, 100 nM AF488-labeled human IgG antibodies and serial dilutions of IgG-based degrader (0.1% DMSO final concentration) were added to the cells and the mixture was incubated at 37° C. for 6 hours. For studies using DNP, 100 nM AF488-labeled DNP antibodies and serial dilutions of DNP-based degrader (0.1% DMSO final concentration) were added to the cells and the mixture was incubated at 37° C. for 6 hours. HepG2 cells were fixed in 100% methanol at −20° C. for 20 min, washed 3× with PBS, then stained with DAPI (Thermo, Cat #H3570). HepG2 cells were imaged for DAPI and Alexa Fluor 488 using the Operetta CLS, high-content analysis system (PerkinElmer). Data is presented as the ratio of the number of cells positive for hIgG or DNP (AF-488 fluorescence) over the total cell number (measured by DAPI).

TABLE 3
ASGPR Binding and IgG Degradation Activity of Select Compounds

| | | ASGPR | IgG | Ter- | |
|---|---|---|---|---|---|
| | | SPR | SPR | nary | |
| | | Bind- | Bind- | Com- | |
| | | ing | ing | plex | Uptake |
| Cmp | | K_{D} | K_{D} | EC₅₀ | Assay |
| Name | Structure | (nM) | (nM) | (nM) | (EC₅₀) |
| 24 | 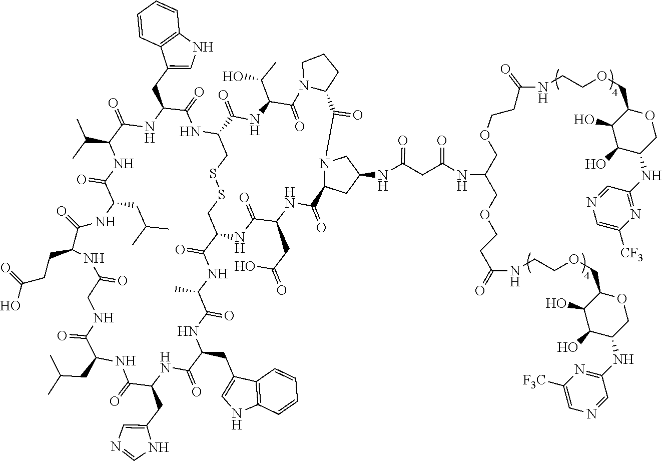 | **** | | | |
| 25 | 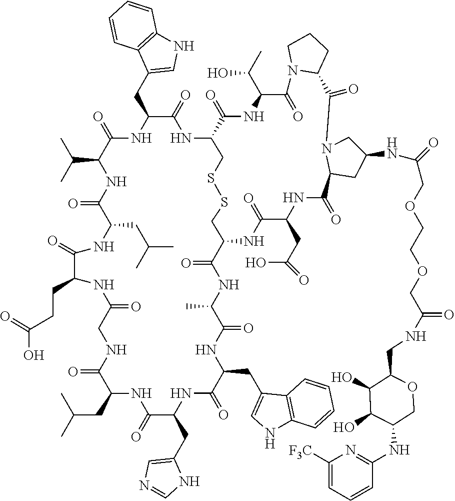 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)COCCOCC(=O)NC[C@H]3OC[C@H](Nc4cccc(C(F)(F)F)n4)[C@@H](O)[C@H]3O)C[C@H]1C(=O)N[C@@H](CC(=O)O)C(=O)N2] | ** | | **** | **** |
| 26 | 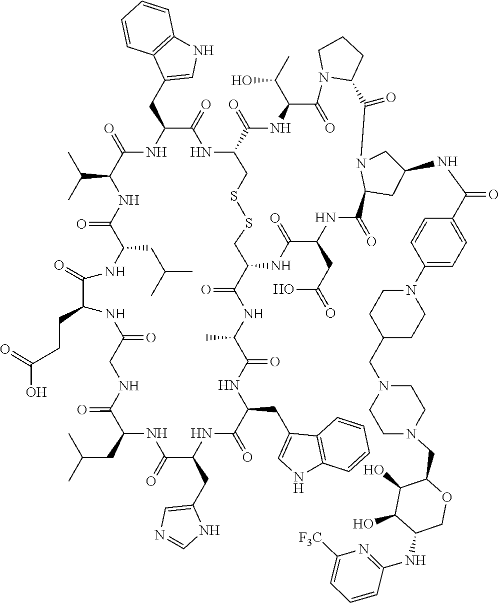 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)c3ccc(N4CCC(CN5CCN(C[C@H]6OC[C@H](Nc7cccc(C(F)(F)F)n7)[C@@H](O)[C@H]6O)CC5)CC4)cc3)C[C@H]1C(=O)N[C@@H](CC(=O)O)C(=O)N2] | **** | **** | **** | **** |
| 27 | 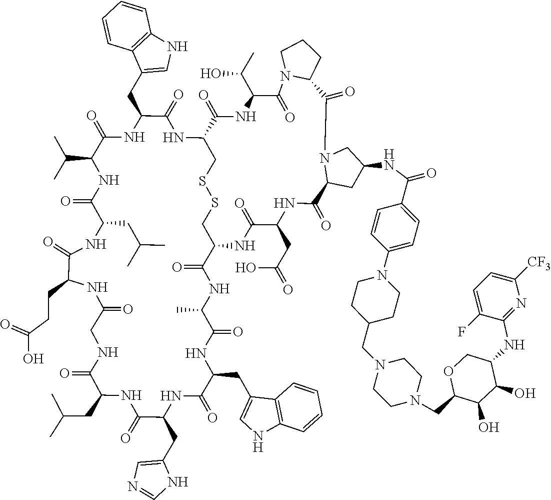 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)c3ccc(N4CCC(CN5CCN(C[C@H]6OC[C@H](Nc7nc(C(F)(F)F)ccc7F)[C@@H](O)[C@H]6O)CC5)CC4)cc3)C[C@H]1C(=O)N[C@@H](CC(=O)O)C(=O)N2] | **** | **** | **** | **** |
| 28 | 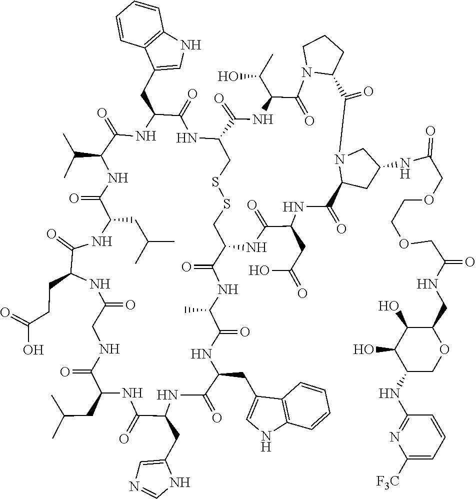 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)COCCOCC(=O)NC[C@H]3OC[C@H](Nc4cccc(C(F)(F)F)n4)[C@@H](O)[C@H]3O)C[C@H]1C(=O)N[C@@H](CC(=O)O)C(=O)N2] | **** | **** | **** | >1100 |
| 29 | 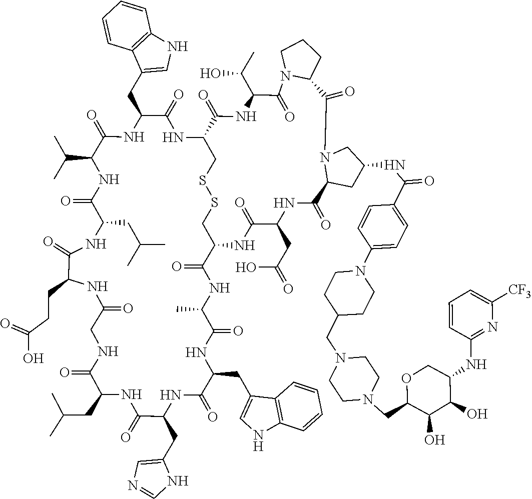 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)c3ccc(N4CCC(CN5CCN(C[C@H]6OC[C@H](Nc7cccc(C(F)(F)F)n7)[C@@H](O)[C@H]6O)CC5)CC4)cc3)C[C@H]1C(=O)N[C@@H](CC(=O)O)C(=O)N2] | **** | **** | **** | *** |
| 30 | 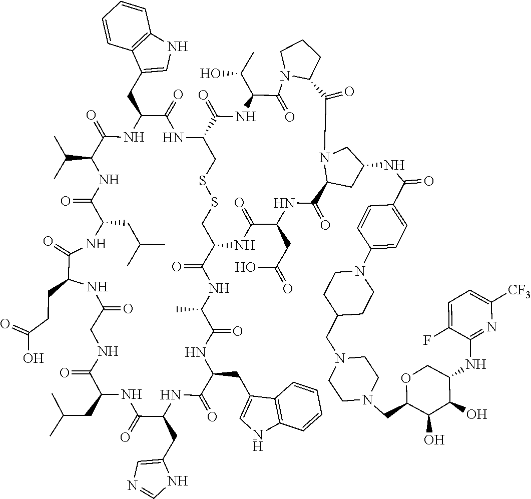 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)c3ccc(N4CCC(CN5CCN(C[C@H]6OC[C@H](Nc7nc(C(F)(F)F)ccc7F)[C@@H](O)[C@H]6O)CC5)CC4)cc3)C[C@H]1C(=O)N[C@@H](CC(=O)O)C(=O)N2] | **** | **** | *** | *** |
| 31 | 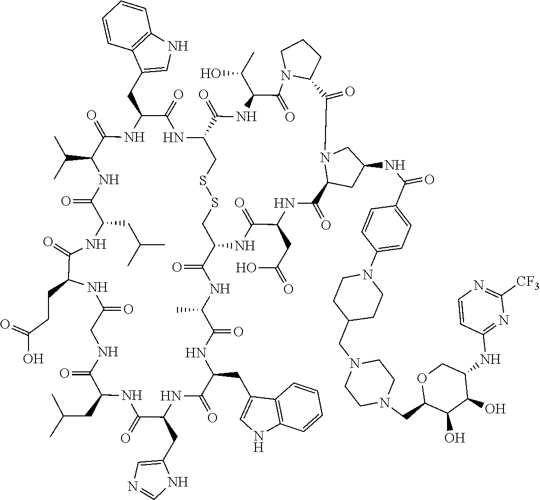 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)c3ccc(N4CCC(CN5CCN(C[C@H]6OC[C@H](Nc7ccnc(C(F)(F)F)n7)[C@@H](O)[C@H]6O)CC5)CC4)cc3)C[C@H]1C(=O)N[C@@H](CC(=O)O)C(=O)N2] | **** | **** | **** | **** |
| 32 | 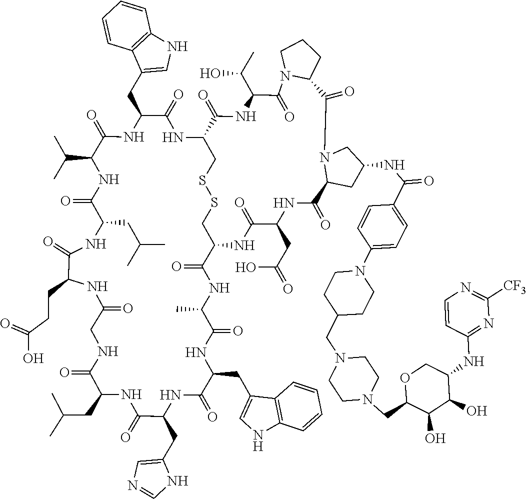 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)c3ccc(N4CCC(CN5CCN(C[C@H]6OC[C@H](Nc7ccnc(C(F)(F)F)n7)[C@@H](O)[C@H]6O)CC5)CC4)cc3)C[C@H]1C(=O)N[C@@H](CC(=O)O)C(=O)N2] | **** | **** | **** | **** |
| 33 | 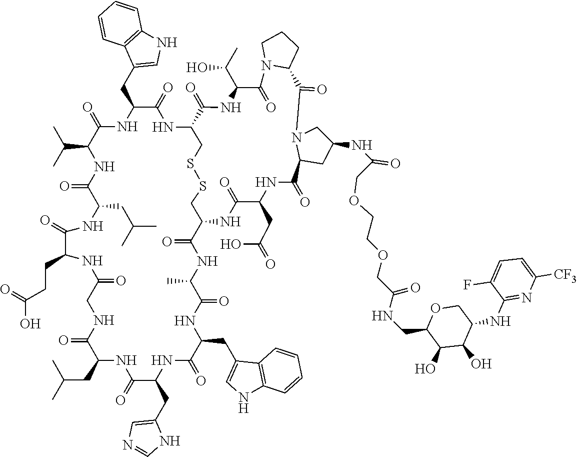 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)COCCOCC(=O)NC[C@H]3OC[C@H](Nc4nc(C(F)(F)F)ccc4F)[C@@H](O)[C@H]3O)C[C@H]1C(=O)N[C@@H](CC(=O)O)C(=O)N2] | *** | **** | **** | **** |
| 34 | 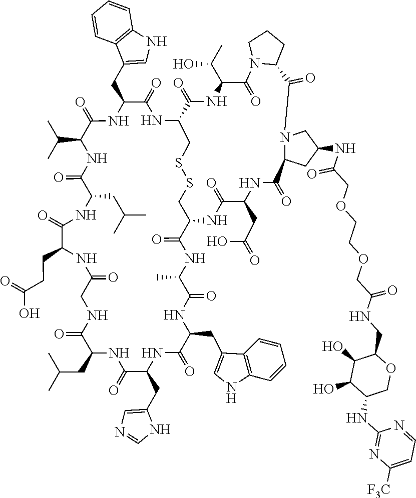 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)COCCOCC(=O)NC[C@H]3OC[C@H](Nc4nccc(C(F)(F)F)n4)[C@@H](O)[C@H]3O)C[C@H]1C(=O)N[C@@H](CC(=O)O)C(=O)N2] | * | **** | >964 | * |
| 35 | 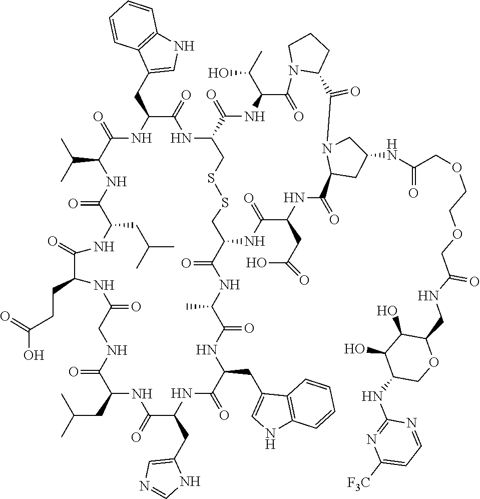 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)COCCOCC(=O)NC[C@H]3OC[C@H](Nc4nccc(C(F)(F)F)n4)[C@@H](O)[C@H]3O)C[C@H]1C(=O)N[C@@H](CC(=O)O)C(=O)N2] | * | **** | >10000 | >10000 |
| 36 | 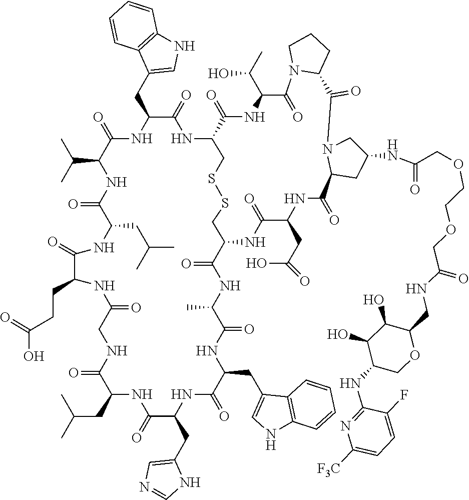 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)COCCOCC(=O)NC[C@H]3OC[C@H](Nc4nc(C(F)(F)F)ccc4F)[C@@H](O)[C@H]3O)C[C@H]1C(=O)N[C@@H](CC(=O)O)C(=O)N2] | | | | |

In the table above, Kd values that are >1000 nM=*, <1000-500 nM=**, <500-100 nM=***, and <100=****

| | | | IgG K_{D} | | |
|---|---|---|---|---|---|
| Cmpd Name | Structure | ASGPR K_{D} (nM) | (nM) | TCF EC₅₀ | Uptake EC₅₀ |
| 37 | 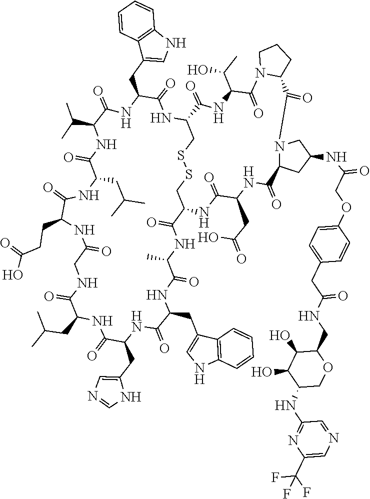 | **** | **** | **** | **** |
| 38 | 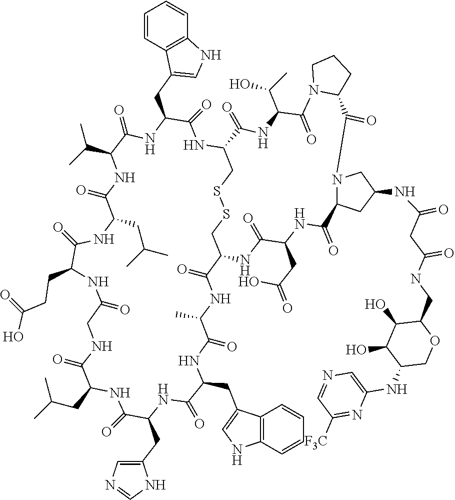 | **** | **** | **** | **** |
| 39 | 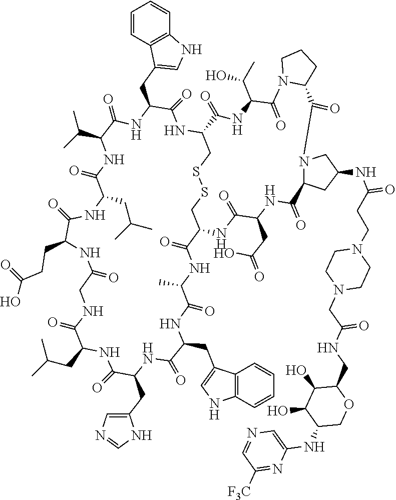 | **** | **** | **** | **** |
| 40 | 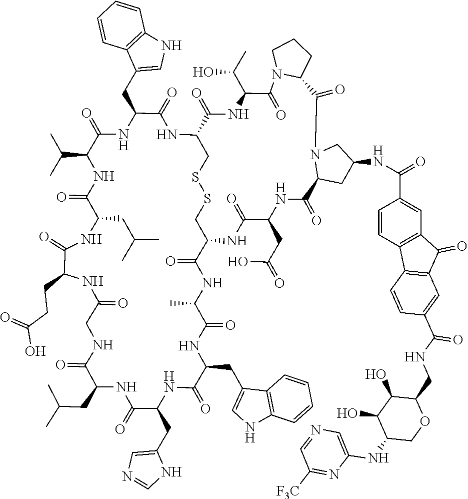 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)c3ccc4c(c3)C(=O)c3cc(C(=O)NC[C@H]5OC[C@H](Nc6cncc(C(F)(F)F)n6)[C@@H](O)[C@H]5O)ccc3-4)C[C@H]1C(=O)N[C@@H](CC(=O)O)C(=O)N2] | **** | **** | **** | *** |
| 41 | 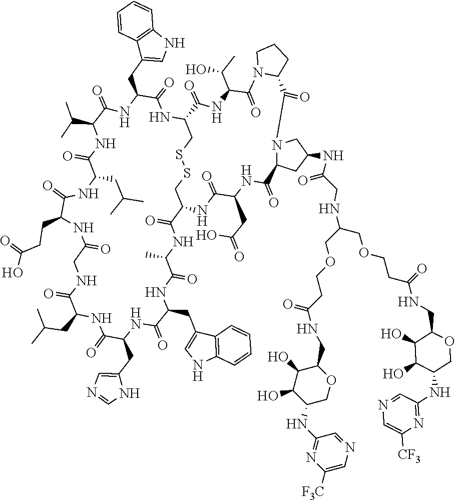 | **** | **** | **** | **** |
| 42 | 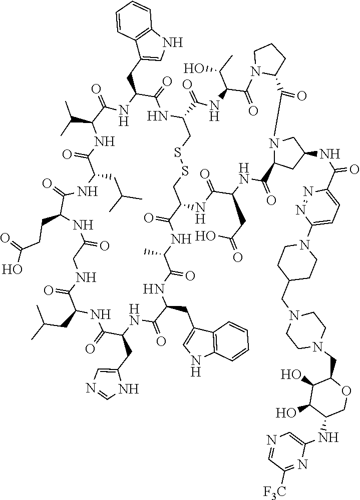 | **** | **** | **** | **** |
| 43 | 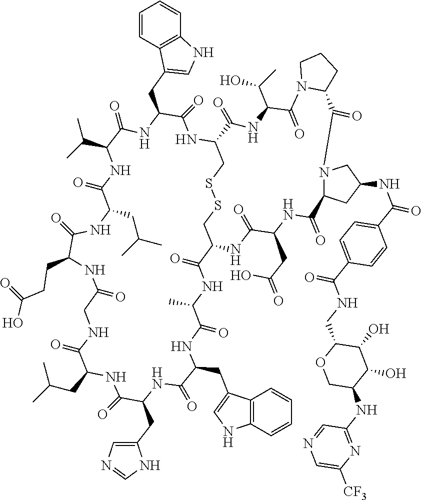 | **** | **** | * | * |
| 44 | 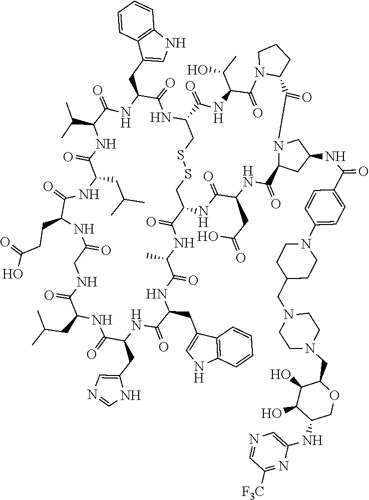 | **** | **** | **** | **** |
| 45 | 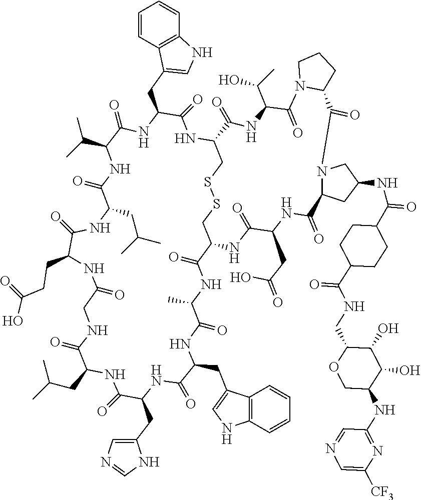 | *** | **** | * | * |
| 46 | 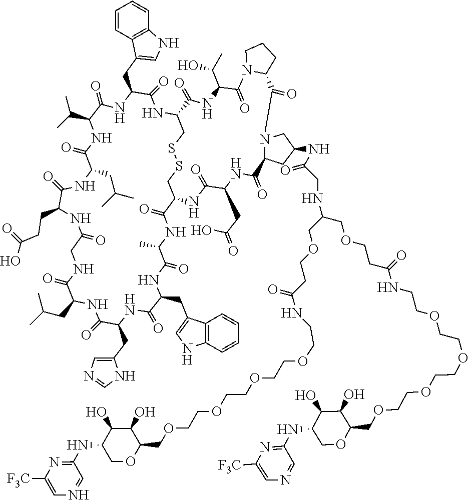 | **** | **** | **** | **** |
| 47 | 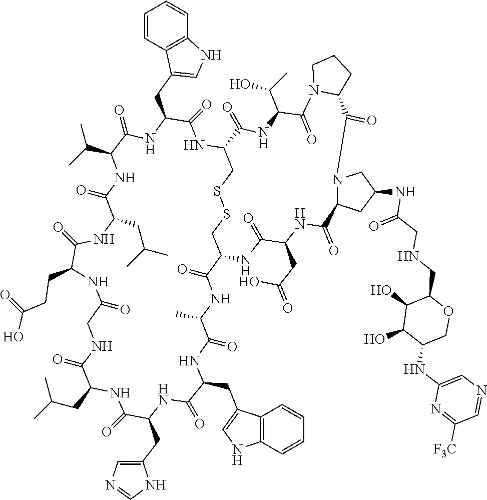 | | | | |
| 48 | 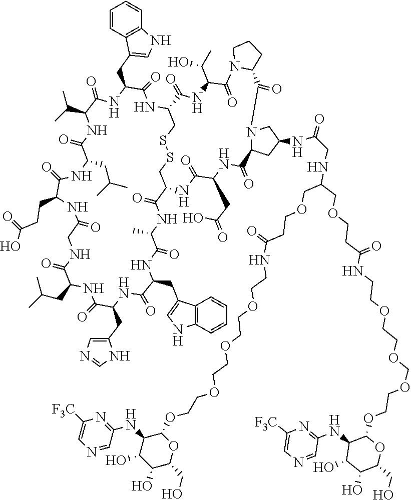 | **** | **** | **** | **** |
| 49 | 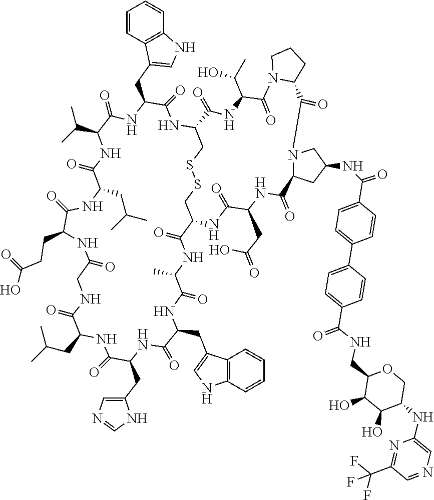 | **** | **** | >686 | **** |
| 50 | 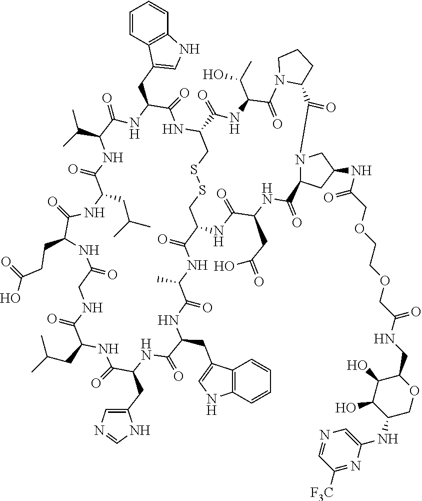 | **** | **** | **** | **** |
| 51 | 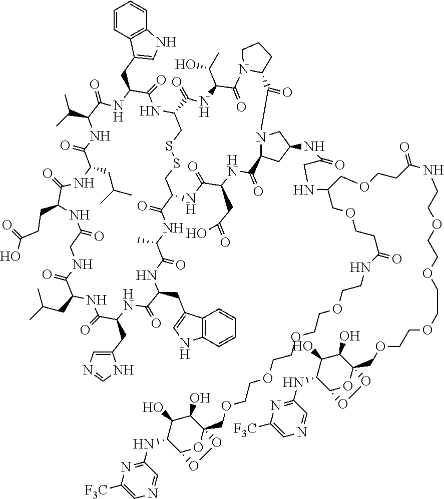 | | | **** | >686 |
| 52 | 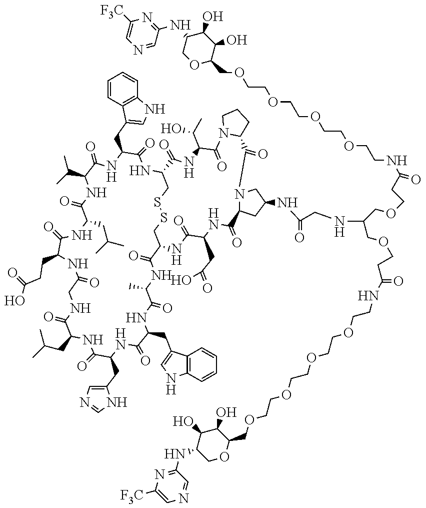 | **** | **** | **** | **** |
| 53 | 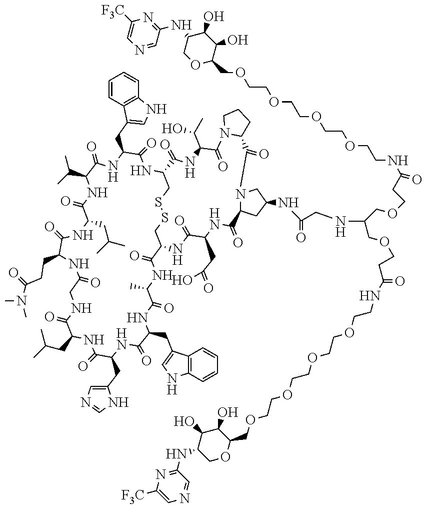 | **** | **** | **** | **** |
| 54 | 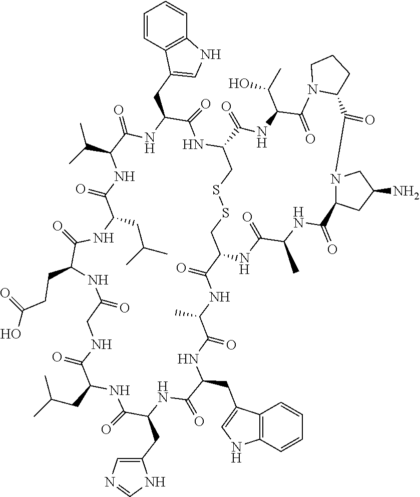 | | **** | | |
| 55 | 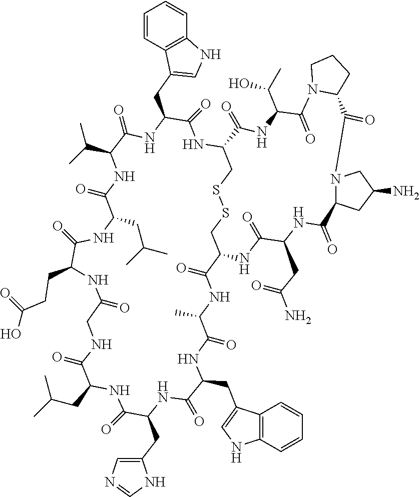 | | **** | | |
| 56 | 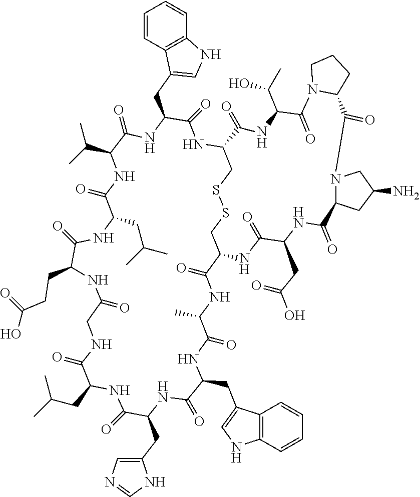 | | **** | | |
| 57 | 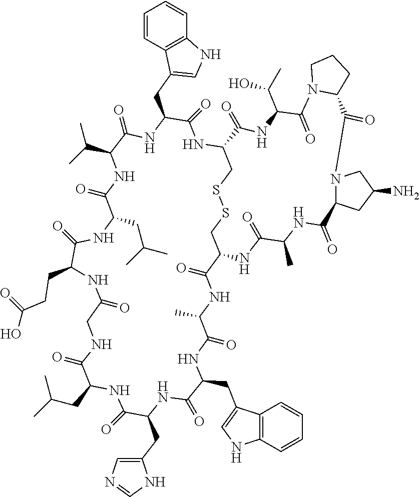 | | **** | | |
| 58 | 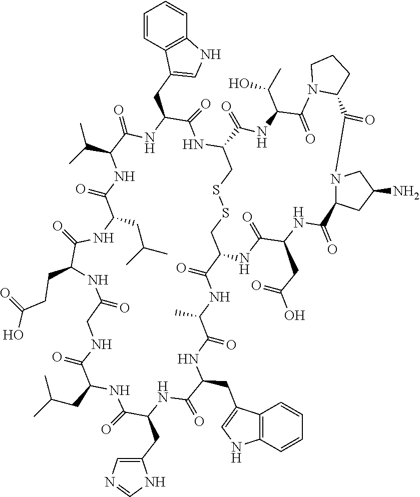 | | **** | | |
| 59 | 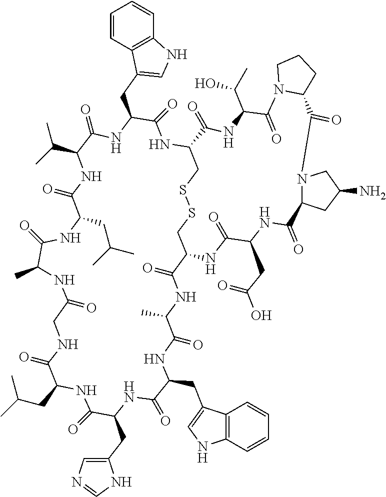 | | **** | | |
| 60 | 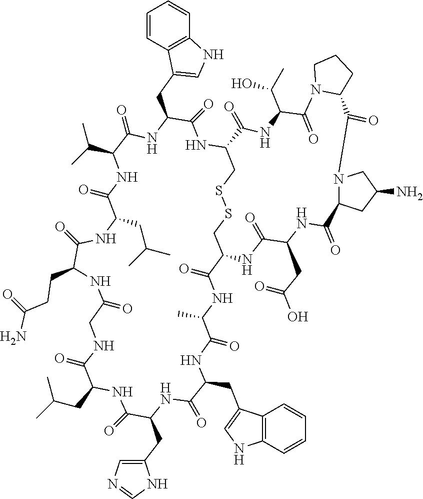 | | **** | | |
| 61 | 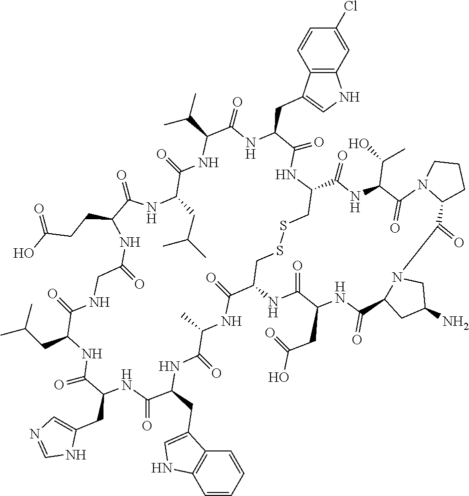 | | **** | | |
| 62 | 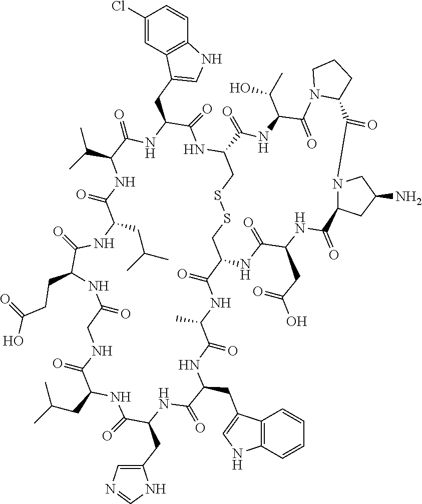 | | **** | | |
| 63 | 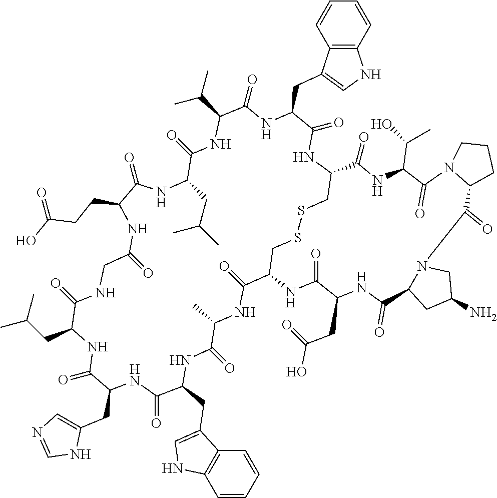 | | **** | | |
| 64 | 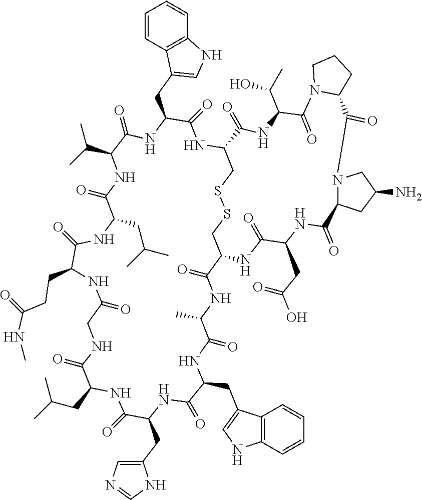 | **** | | | |
| 65 | 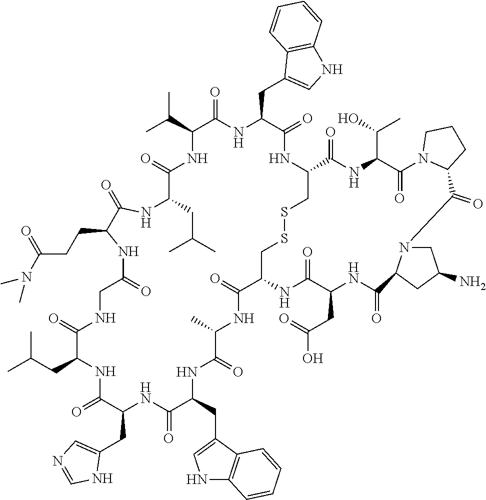 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)N(C)C)NC(=O)CNC1=O)C(=O)N[C@@H]([C@@H](C)O)C(=O)N1CCC[C@@H]1C(=O)N1C[C@@H](N)C[C@H]1C(=O)N[C@@H](CC(=O)O)C(=O)N2] | | **** | | |
| 66 | 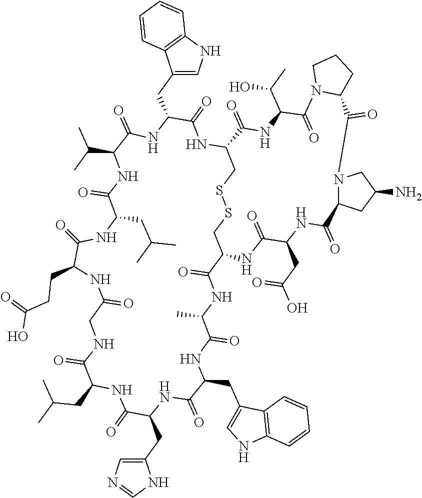 | | **** | | |
| 67 | 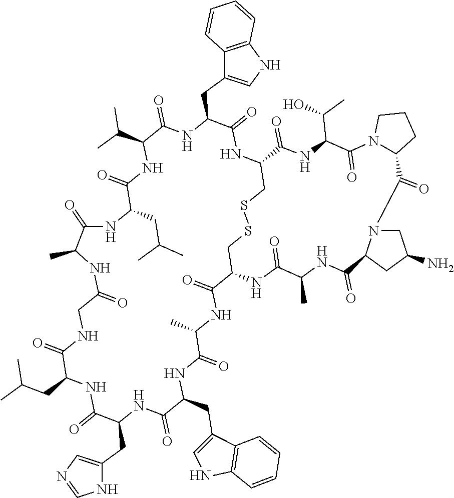 [SMILES: CC(C)C[C@@H]1NC(=O)[C@H](C)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC1=O)C(=O)N[C@@H]([C@@H](C)O)C(=O)N1CCC[C@@H]1C(=O)N1C[C@@H](N)C[C@H]1C(=O)N[C@@H](C)C(=O)N2] | | **** | | |
| 68 | 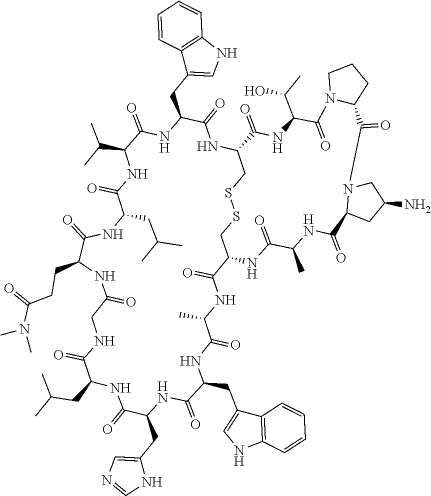 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)N(C)C)NC(=O)CNC1=O)C(=O)N[C@@H]([C@@H](C)O)C(=O)N1CCC[C@@H]1C(=O)N1C[C@@H](N)C[C@H]1C(=O)N[C@@H](C)C(=O)N2] | | **** | | |
| 69 | 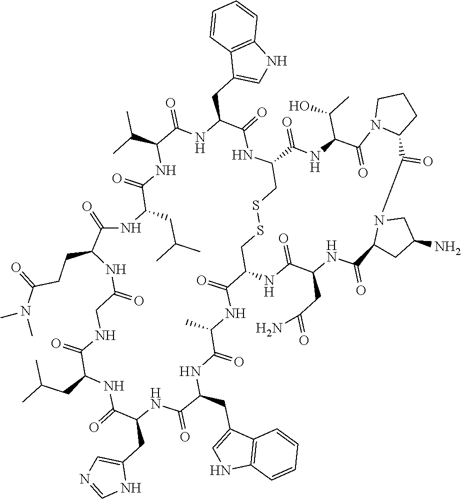 | | **** | | |
| 70 | 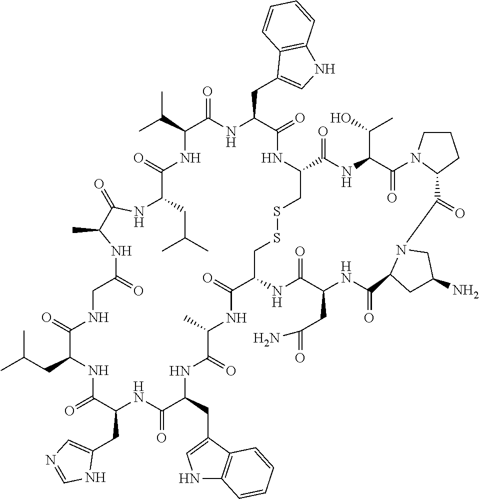 [SMILES: CC(C)C[C@@H]1NC(=O)[C@H](C)NC(=O)CNC(=O)[C@H](CC(C)C)NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC1=O)C(=O)N[C@@H]([C@@H](C)O)C(=O)N1CCC[C@@H]1C(=O)N1C[C@@H](N)C[C@H]1C(=O)N[C@@H](CC(N)=O)C(=O)N2] | | **** | | |
| 71 | 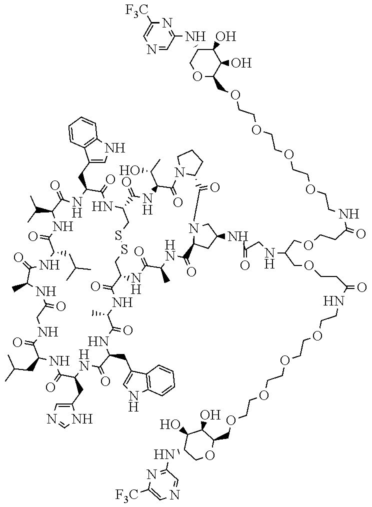 | **** | **** | **** | **** |
| 72 | 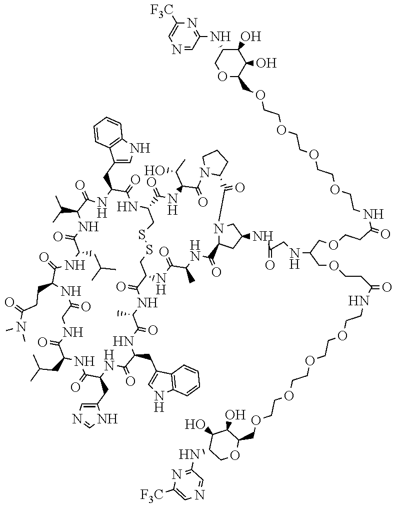 | **** | **** | **** | **** |
| 73 | 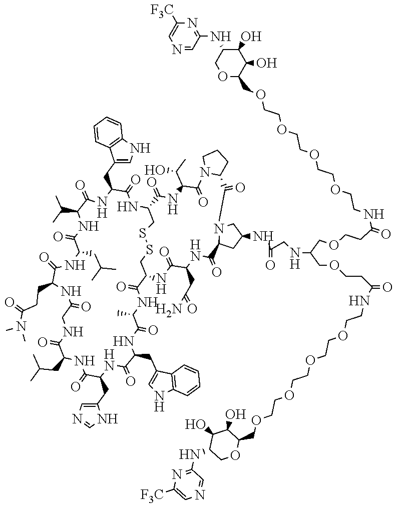 | **** | **** | **** | **** |
| 74 | 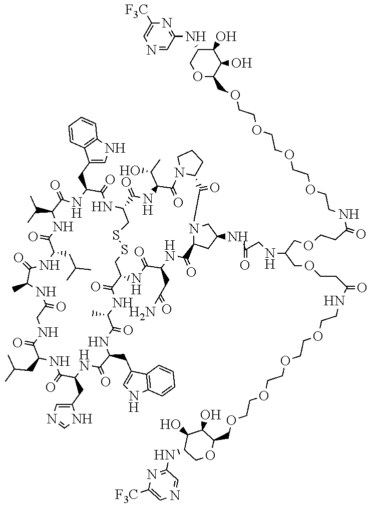 | **** | **** | **** | **** |
| 75 | 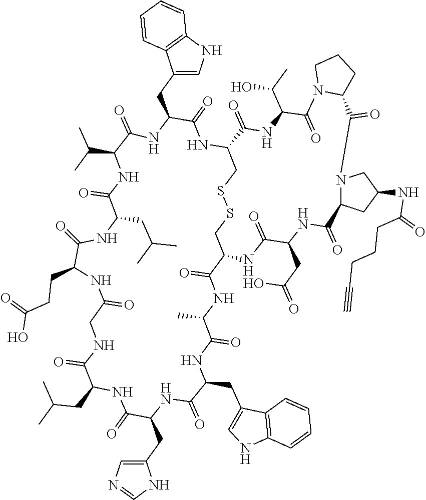 | | **** | | |
| 76 | 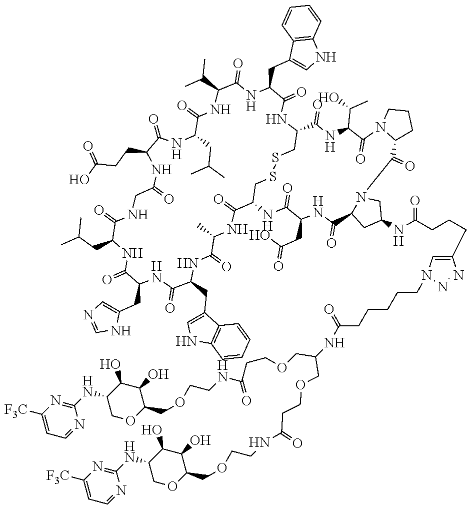 | **** | **** | **** | **** |
| 77 | 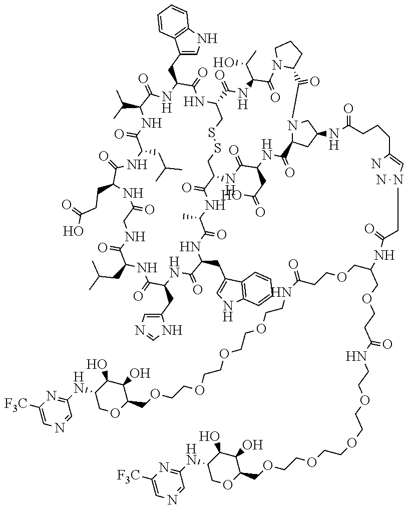 | | **** | **** | **** |
| 78 | 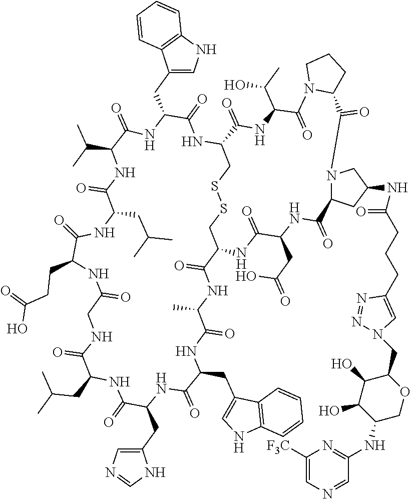 | **** | **** | **** | **** |
| 79 | 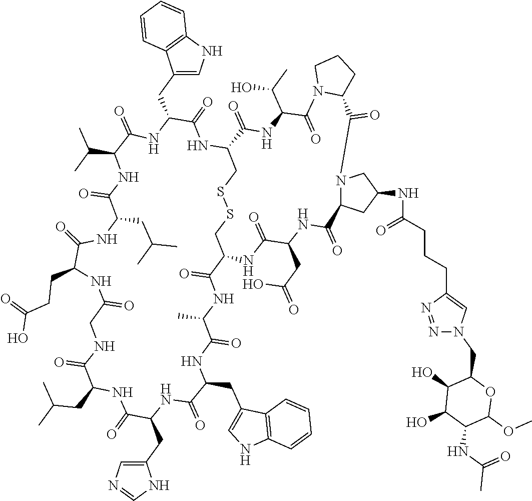 | * | **** | | |
| 80 | 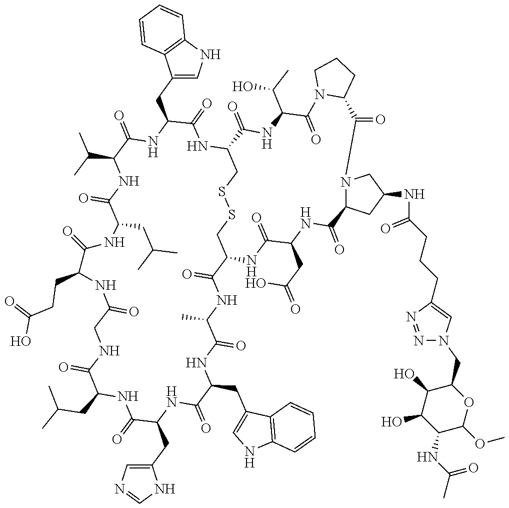 [SMILES: COC1O[C@H](Cn2cc(CCCC(=O)N[C@H]3C[C@H]4C(=O)N[C@@H](CC(=O)O)C(=O)N[C@H]5CSSC[C@H](NC(=O)[C@H](Cc6c[nH]c7ccccc67)NC(=O)[C@H](C(C)C)NC(=O)[C@H](CC(C)C)NC(=O)[C@H](CCC(=O)O)NC(=O)CNC(=O)[C@H](CC(C)C)NC(=O)[C@H](Cc6cnc[nH]6)NC(=O)[C@H](Cc6c[nH]c7ccccc67)NC(=O)[C@H](C)NC5=O)C(=O)N[C@@H]([C@@H](C)O)C(=O)N5CCC[C@@H]5C(=O)N4C3)nn2)[C@H](O)[C@H](O)[C@H]1NC(C)=O] | | | >4360 | >10000 |
| 81 | 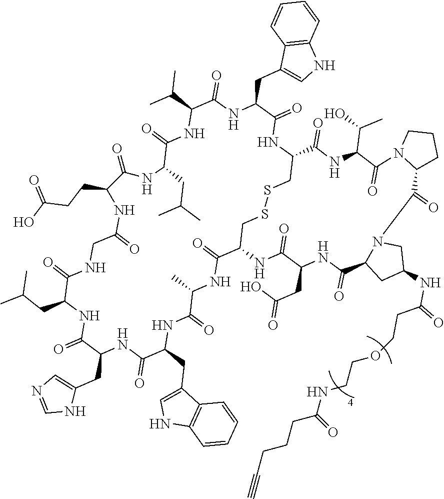 | | **** | | |
| 82 | 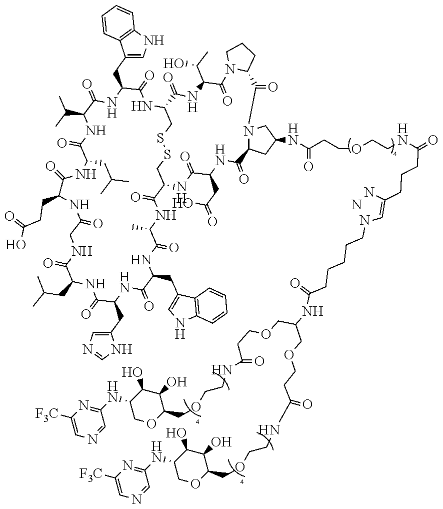 | **** | **** | **** | **** |
| 83 | 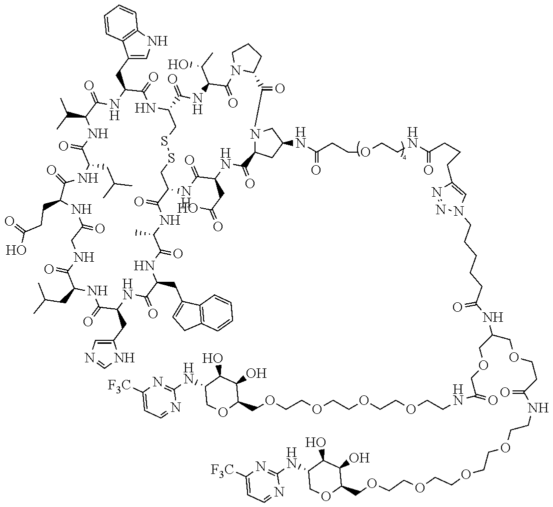 | **** | **** | **** | **** |
| 84 | 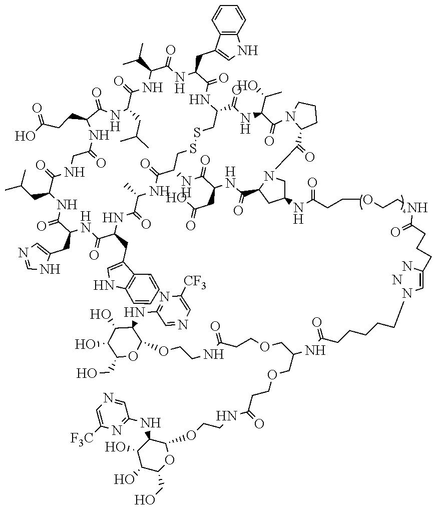 | | | **** | **** |
| 85 | 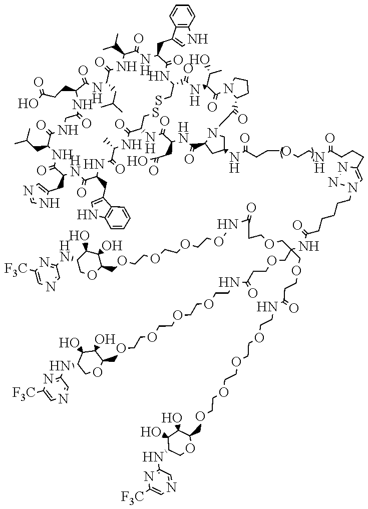 | | | **** | **** |
| 86 | 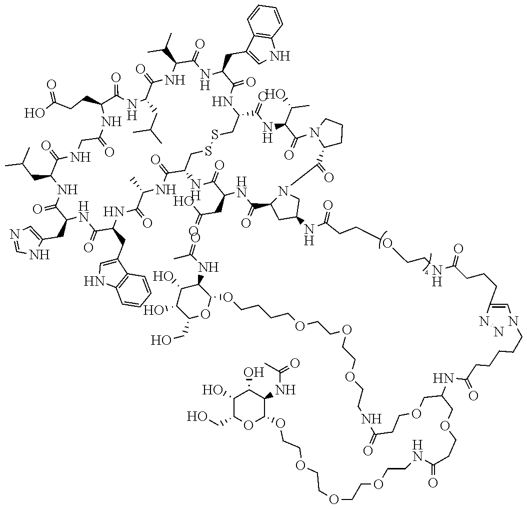 | * | **** | * | * |
| 87 | 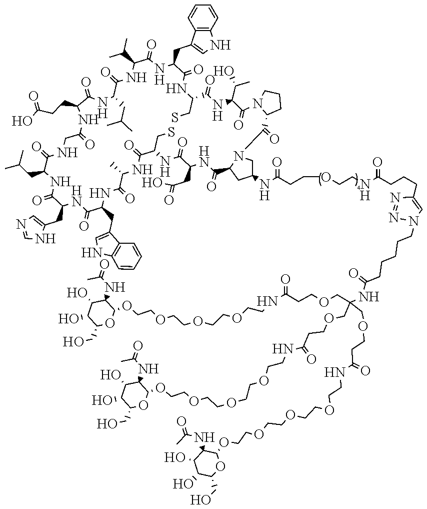 | * | **** | * | * |
| 88 | 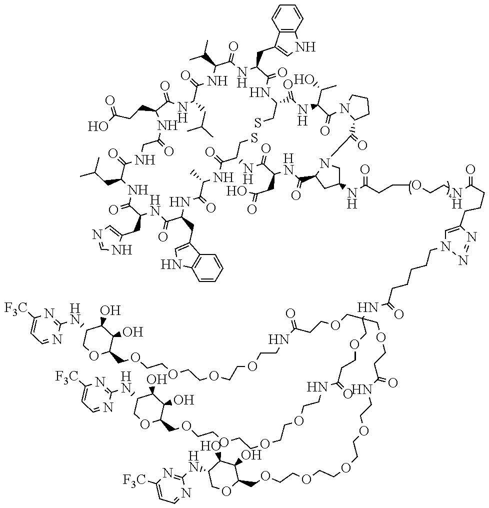 | **** | **** | **** | **** |
| 89 | 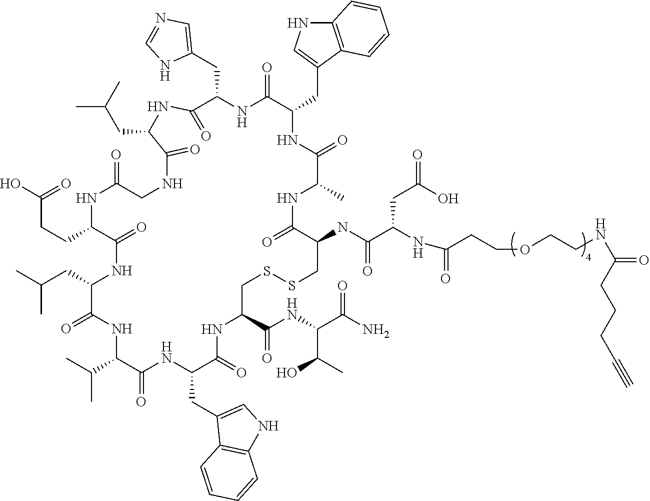 | | **** | | |
| 90 | 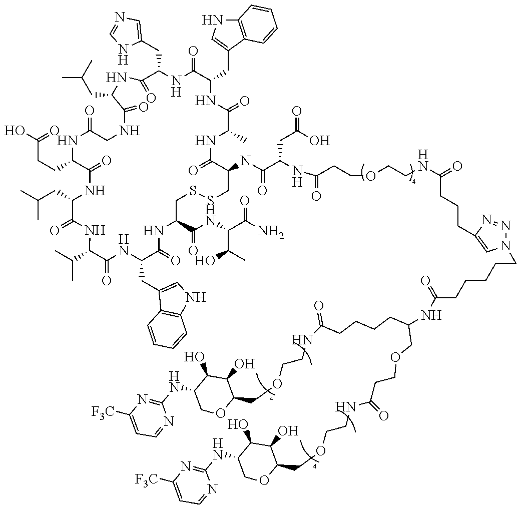 | **** | **** | **** | **** |
| 91 | 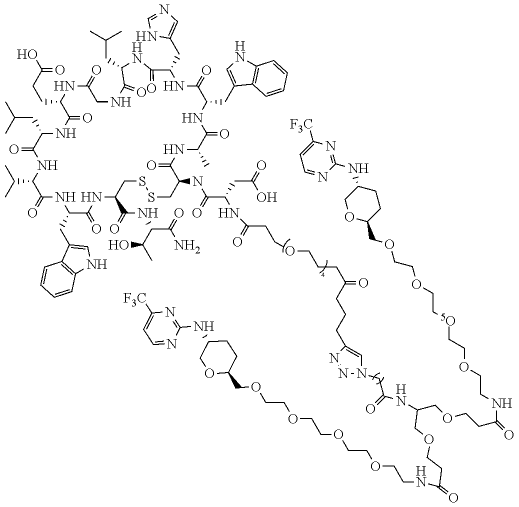 | * | | * | * |
| 92 | 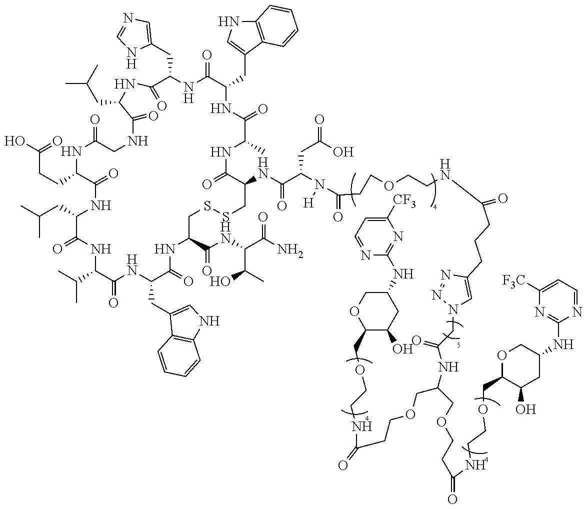 | * | | * | * |
| 93 | 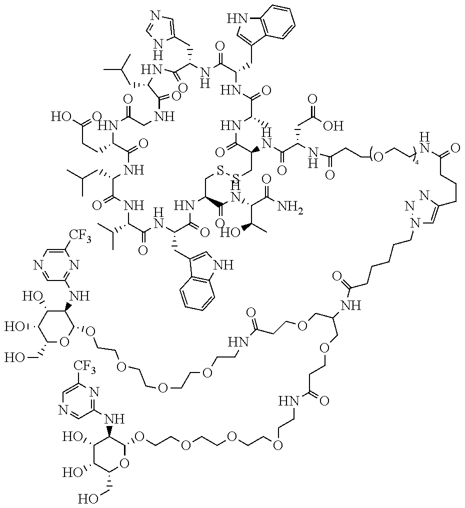 | **** | **** | **** | **** |
| 94 | 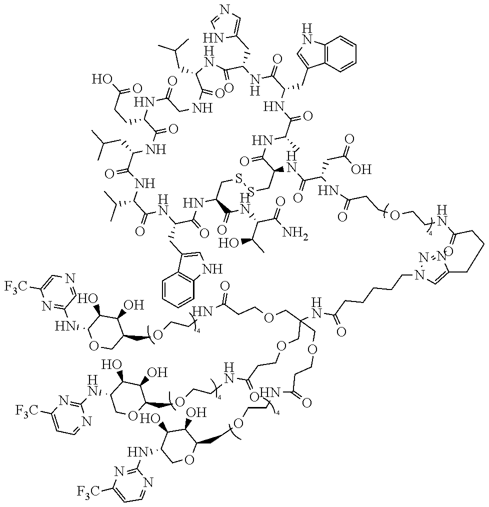 | **** | | **** | **** |
| 95 | 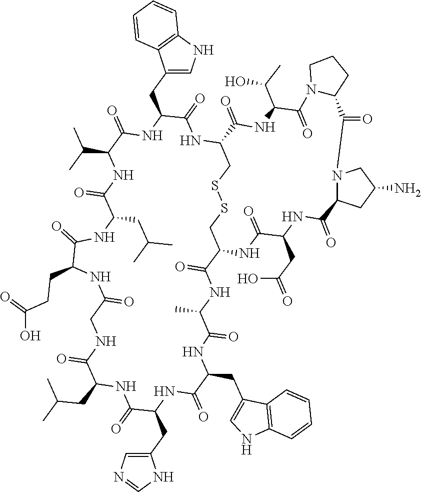 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](N)C[C@H]1C(=O)N[C@@H](CC(=O)O)C(=O)N2] | | **** | | |
| 96 | 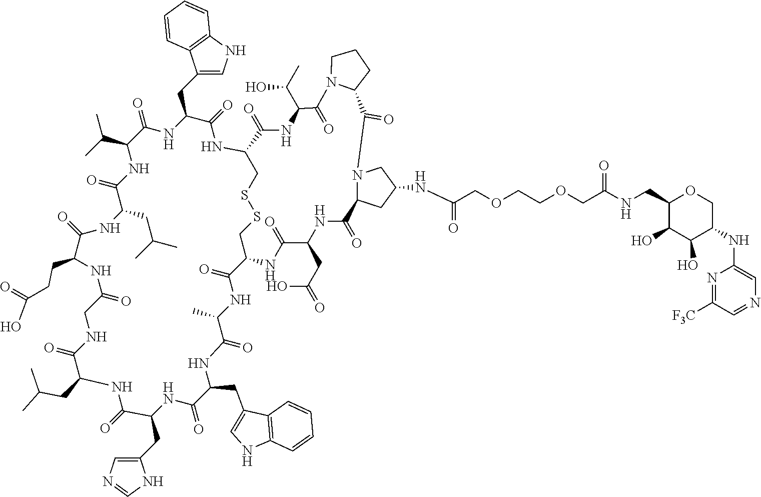 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)COCCOCC(=O)NC[C@H]3OC[C@H](Nc4cncc(C(F)(F)F)n4)[C@@H](O)[C@H]3O)C[C@H]1C(=O)N[C@@H](CC(=O)O)C(=O)N2] | **** | **** | **** | **** |
| 97 | 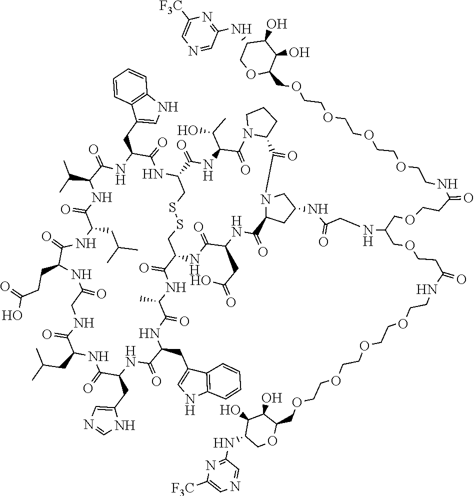 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@H](NC(=O)CNC(COCCC(=O)NCCOCCOCCOCCOC[C@H]3OC[C@H](Nc4cncc(C(F)(F)F)n4)[C@@H](O)[C@H]3O)COCCC(=O)NCCOCCOCCOCCOC[C@H]3OC[C@H](Nc4cncc(C(F)(F)F)n4)[C@@H](O)[C@H]3O)C[C@H]1C(=O)N[C@@H](CC(=O)O)C(=O)N2] | **** | **** | **** | **** |
| 98 | 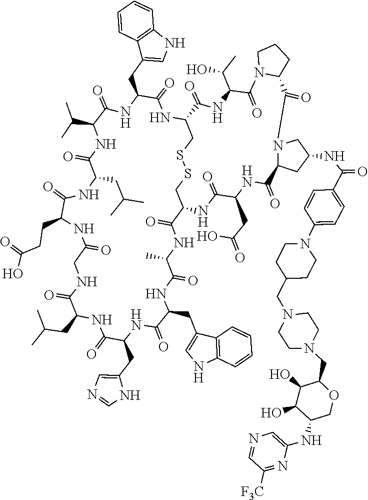 | | **** | **** | **** |
| 99 | 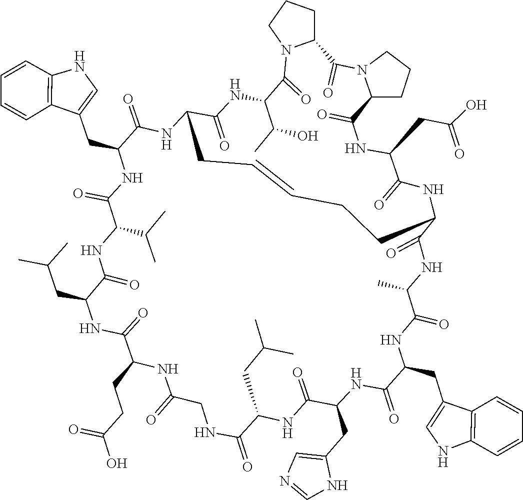 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CC/C=C/C[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1CCC[C@H]1C(=O)N[C@@H](CC(=O)O)C(=O)N2] | | **** | | |
| 100 | 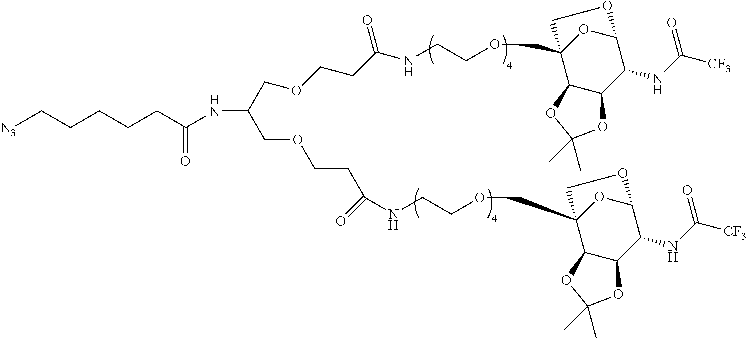 | | | | |
| 101 | 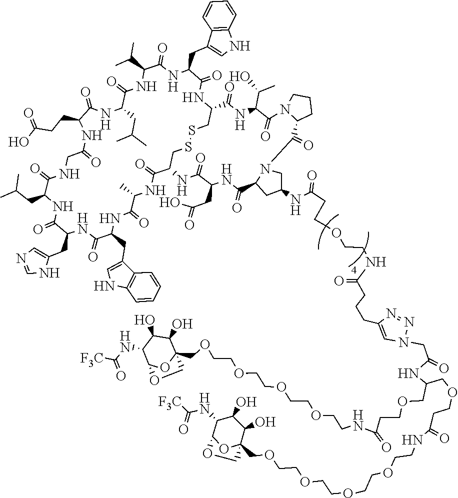 | **** | **** | **** | **** |
| 102 | 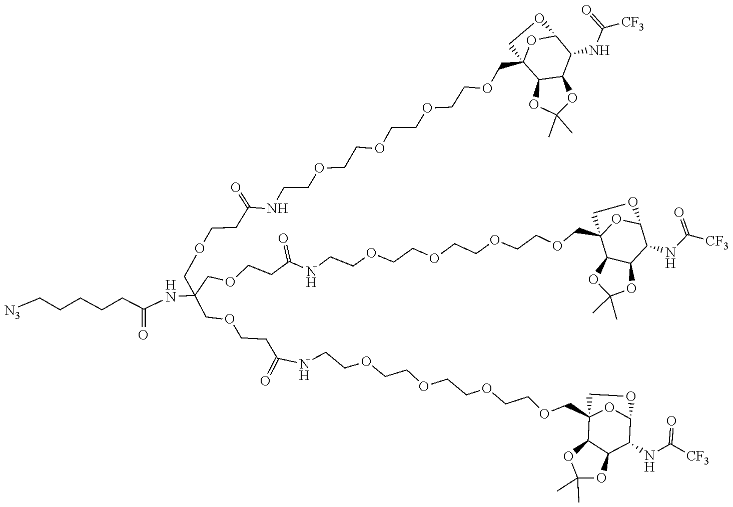 [SMILES: CC1(C)O[C@@H]2[C@@H](NC(=O)C(F)(F)F)[C@H]3OC[C@](COCCOCCOCCOCCNC(=O)CCOCC(COCCC(=O)NCCOCCOCCOCCOC[C@@]45CO[C@@H](O4)[C@H](NC(=O)C(F)(F)F)[C@H]4OC(C)(C)O[C@H]45)(COCCC(=O)NCCOCCOCCOCCOC[C@@]45CO[C@@H](O4)[C@H](NC(=O)C(F)(F)F)[C@H]4OC(C)(C)O[C@H]45)NC(=O)CCCCCN=[N+]=[N-])(O3)[C@@H]2O1] | | | | |
| 103 | 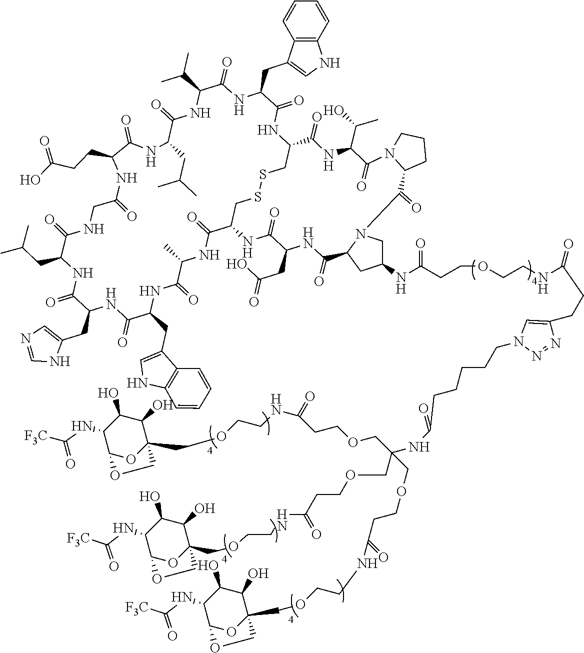 | **** | **** | **** | **** |
| 104 | 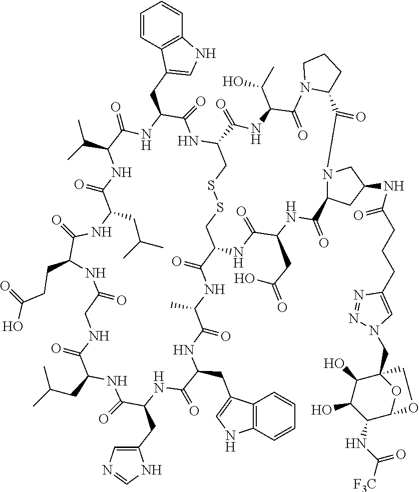 | | **** | >10000 | >10000 |
| 105 | 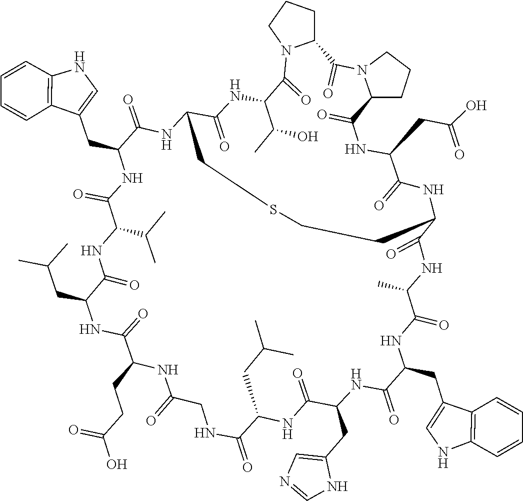 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CCSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1CCC[C@H]1C(=O)N[C@@H](CC(=O)O)C(=O)N2] | | | | |
| 106 | 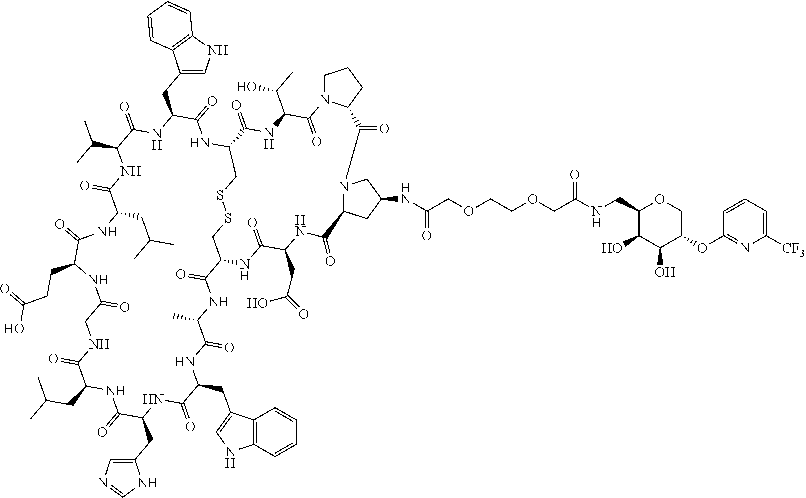 [SMILES: CC(C)C[C@@H]1NC(=O)[C@H](Cc2cnc[nH]2)NC(=O)[C@H](Cc2c[nH]c3ccccc23)NC(=O)[C@H](C)NC(=O)[C@@H]2CSSC[C@H](NC(=O)[C@H](Cc3c[nH]c4ccccc34)NC(=O)[C@H](C(C)C)NC(=O)[C@H](CC(C)C)NC(=O)[C@H](CCC(=O)O)NC(=O)CNC1=O)C(=O)N[C@@H]([C@@H](C)O)C(=O)N1CCC[C@@H]1C(=O)N1C[C@@H](NC(=O)COCCOCC(=O)NC[C@H]3OC[C@H](Oc4cccc(C(F)(F)F)n4)[C@@H](O)[C@H]3O)C[C@H]1C(=O)N[C@@H](CC(=O)O)C(=O)N2] | | | | |
| 107 | 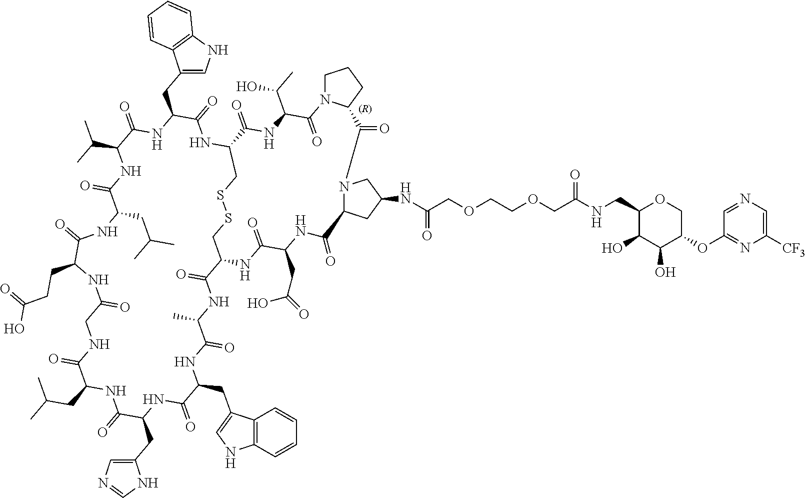 | | | | |
| 108 | 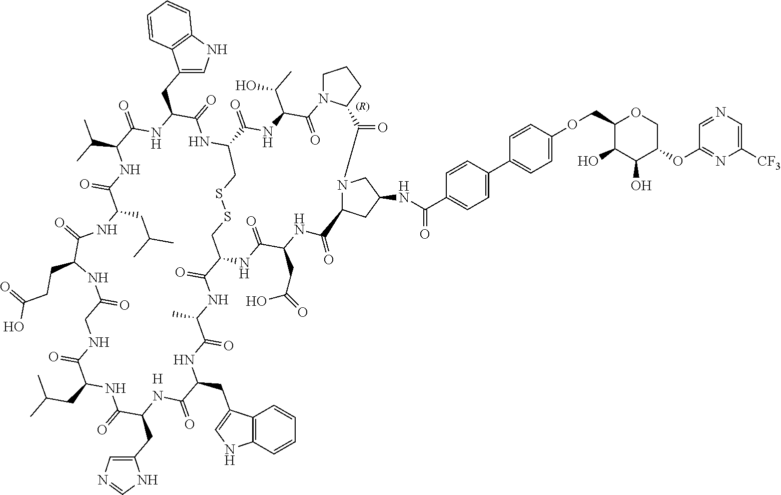 | | | | |
| 109 | 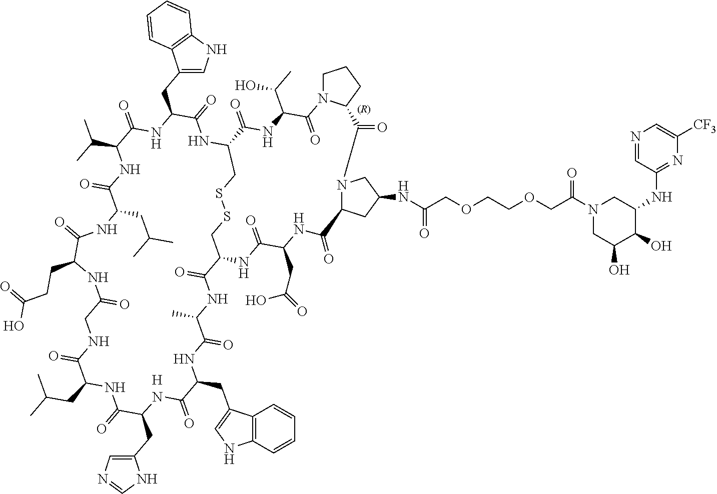 | | | | |

In the table above, values that are >1000 nM=*, <1000-500 nM=**, <500-100 nM=***, and <100=****

### VI. Processes of Manufacture

The extracellular protein degraders of the present invention can be manufactured according to routes described in the Working Examples below or as otherwise known in the patent or scientific literature and if appropriate supported by the knowledge of the ordinary worker or common general knowledge.

Some of the carbons in the extracellular protein degraders described herein are drawn with designated stereochemistry. Other carbons are drawn without stereochemical designation. When drawn without designated stereochemistry, that carbon can be in any desired stereochemical configuration that achieves the desired purpose. One skilled in the art will recognize that pure enantiomers, enantiomerically enriched compounds, racemates and diastereomers can be prepared by methods known in the art as guided by the information provided herein. Examples of methods to obtain optically active materials include at least the following:
i) chiral liquid chromatography—a technique whereby diastereomers are separated in a liquid mobile phase by virtue of their differing interactions with a stationary phase (including vial chiral HPLC). The stationary phase can be made of chiral material or the mobile phase can contain an additional chiral material to provoke the differing interactions;
ii) non-chiral chromatography of diastereomers—Often diastereomers can be separated using normal non-chiral column conditions;
iii) chiral gas chromatography—a technique whereby the racemate is volatilized and enantiomers are separated by virtue of their differing interactions in the gaseous mobile phase with a column containing a fixed non-racemic chiral adsorbent phase;
iv) simultaneous crystallization—a technique whereby the individual diastereomers are separately crystallized from a solution;
v) enzymatic resolutions—a technique whereby partial or complete separation of diastereomers are separated by virtue of differing rates of reaction with an enzyme;
vi) chemical asymmetric synthesis—a synthetic technique whereby the desired diastereomer is synthesized from an achiral precursor under conditions that produce asymmetry (i.e. chirality) in the product, which may be achieved by chiral catalysts or chiral auxiliaries;
vii) diastereomer separations—a technique whereby a racemic compound is reaction with an enantiomerically pure reagent (the chiral auxiliary) that converts the individual enantiomers to diastereomers. The resulting diastereomers are then separated by chromatography or crystallization by virtue of their now more distinct structural differences the chiral auxiliary later removed to obtain the desired enantiomer; and
viii) extraction with chiral solvents—a technique whereby diastereomers are separated by virtue of preferential dissolution of one over the others in a particular chiral solvent.

### General Procedures Applied to the Working Examples of Synthesis

All reagents were purchased from commercial suppliers (Sigma-Aldrich, Alfa, Acros etc.) and used without further purification unless otherwise stated. THE was continuously refluxed and freshly distilled from sodium and benzophenone under nitrogen, dichloromethane was continuously refluxed and freshly distilled from CaH₂ under nitrogen.

Reactions were monitored via TLC on silica gel 60 HSGF254 percolated plates (0.15-0.2 mm SiO₂) and visualized using UV light (254 nm or 365 nm) and/or staining with phosphomolybdic acid ethanol solution (10 g in 100 mL ethanol) and subsequent heating or monitored via LCMS.

LCMS were performed on SHIMADZU LCMS-2010EV (Chromolith SpeedROD, RP-18e, 50×4.6 mm, mobile phase: Solvent A: CH₃CN/H₂O/HCOOH=10/90/0.05, Solvent B: CH₃CN/H₂O/HCOOH=90/10/0.05, 0.8 min@ 10% B, 2.7 min gradient (10-95% B), then 0.8 min@95% B, Flow rate: 3 mL/min, temperature: 40° C.).

Preparative HPLC were performed either on Method A: SHIMADZU LC-8A (Column: YMC Pack ODS-A (150*30 mm, 10 μm)) or Method B: LC-6AD (Column: Shim=Pack PREP-ODS-H (250*20 mm, 10 μm)) with UV detection which were controlled by LC solution Chemstation software. H₂O (0.1% HCOOH) and MeOH (MeCN) as mobile phase at the indicated flow rate.

Analytical HPLC were performed on SHIMADZU LC-2010A (Chromolith SpeedROD, RP-18e, 50×4.6 mm, mobile phase: Solvent A: CH₃CN/H₂O/HCOOH=10/90/0.05, Solvent B: CH₃CN/H₂O/HCOOH=90/10/0.05, 0.8 min@10% B, 2.7 min gradient (10-95% B), then 0.8 min@95% B, Flow rate: 3 mL/min, temperature: 40° C.).

Chiral HPLC were performed on SHIMADZU LC-2010A (Chiral column, mobile phase: Solvent A: hexane (or contained 0.1% diethylamine), Solvent B: Ethanol or Isopropanol; Flow rate: 0.8 mL/min, temperature: 30° C.).

¹H spectra were recorded on Bruker Avance II 400 MHz, Chemical shifts (δ) were reported in ppm relative to tetramethylsilane (δ=0.000 ppm) and the spectra were calibrated to the residual solvent signal of chloroform (δ=7.26), Dimethyl sulfoxide (δ=2.50), methanol (δ=3.30). Data for ¹H NMR spectra were reported as following: chemical shift (multiplicity, number of hydrogens). Abbreviations were described as following: s (singlet), d (doublet), t (triplet), q (quartet), quant (quintet), m (multiple), br (broad).

The reagent OPT-Alkyne has the following structure:

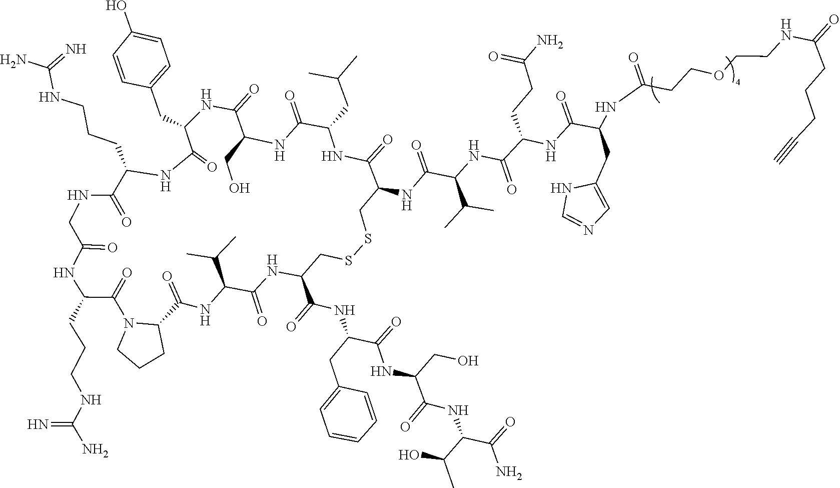

The reagent FC-III peptide-alkyne has the following structure:

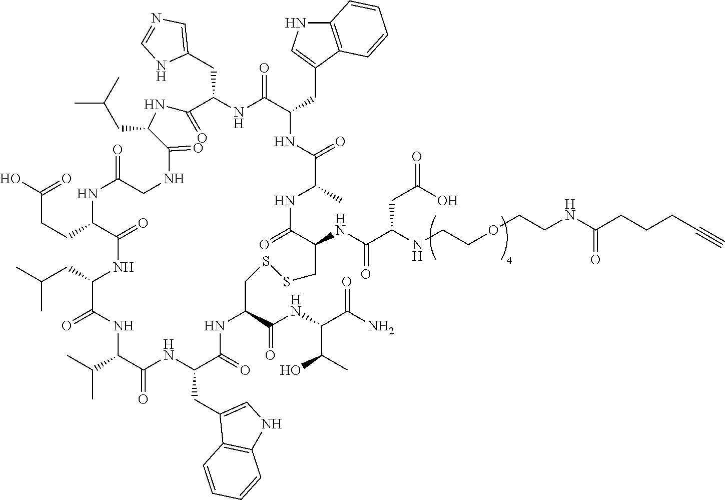

### General Synthetic Methods

The extracellular protein degrading compounds of the present invention can be synthesized using a variety of techniques known to the skilled artisan. For example, starting with an Extracellular Protein Targeting Ligand with an amine the linker and ASGPR binding portion of the molecule can be installed with peptide coupling conditions.

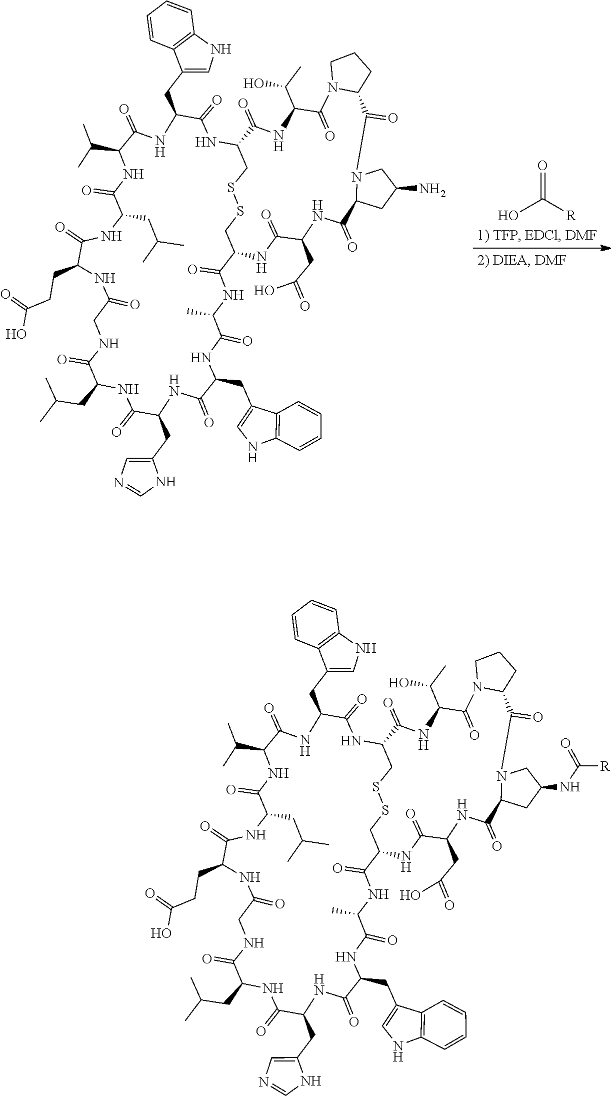

This technique can be used for the synthesis of various extracellular protein degrading compounds of the present invention including those that possess varied stereocenters.

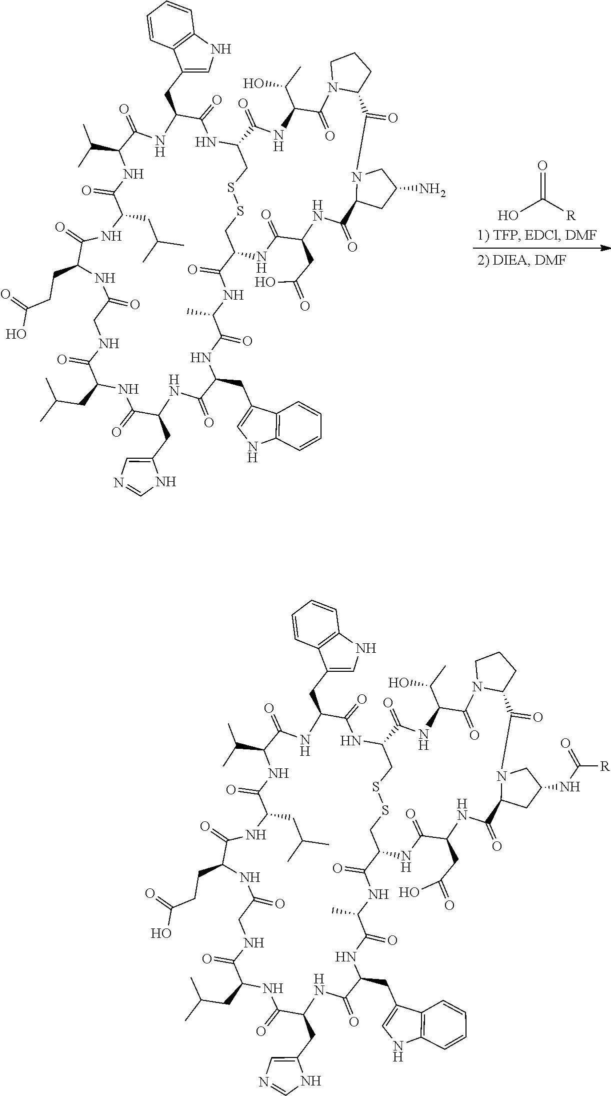

### New C2 Linkage Chemistry

### Preparation of (3R,4R,5R)-2-(acetoxymethyl)-6-((14-azido-3,6,9,12-tetraoxatetradecyl)oxy)-5-((4-(trifluoromethyl)pyrimidin-2-yl)methyl)tetrahydro-2H-pyran-3,4-diyl diacetate

### Step 1: Preparation of (2S)-2-((tert-butyldimethylsilyl)oxy)-2-(2,2-dimethyl-1,3-dioxolan-4-yl)acetaldehyde

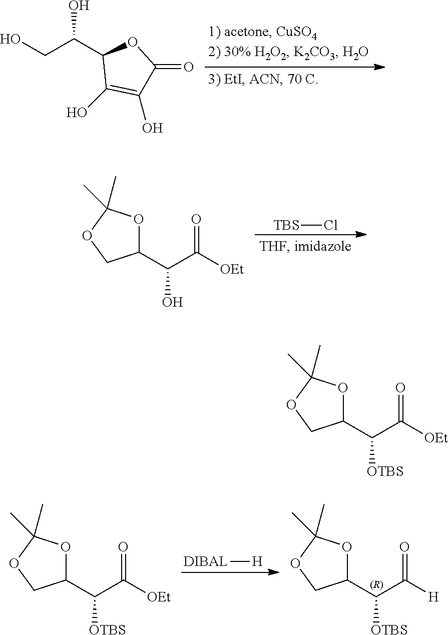

### Step 2: Preparation of (3R,4R,5R)-4,5-dihydroxy-6-(hydroxymethyl)-3-((4-(trifluoromethyl)pyrimidin-2-yl)methyl)tetrahydro-2H-pyran-2-one

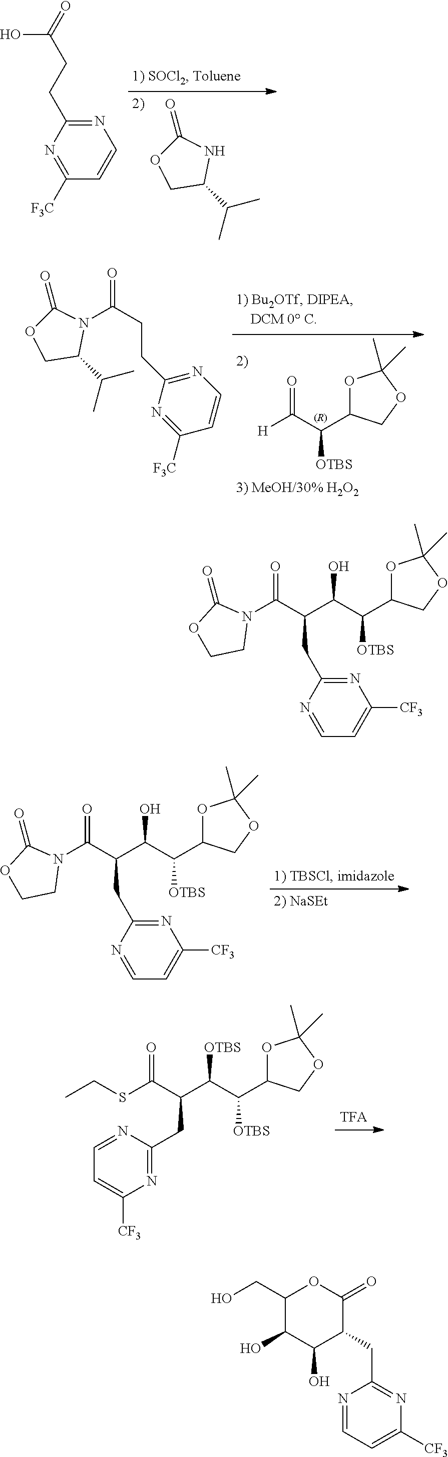

### Preparation of (3R,4R,5R)-2-(acetoxymethyl)-6-((14-azido-3,6,9,12-tetraoxatetradecyl)oxy)-5-((4-(trifluoromethyl)pyrimidin-2-yl)methyl)tetrahydro-2H-pyran-3,4-diyl diacetate

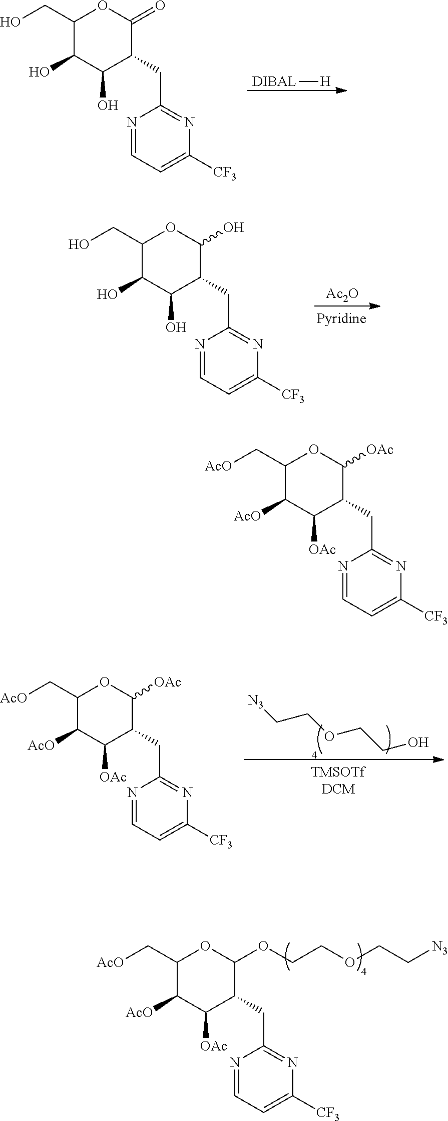

### Preparation of (2R,3R,4S,5S)-2-(hydroxymethyl)-5-(pyrimidin-2-yloxy)tetrahydro-2H-pyran-3,4-diol

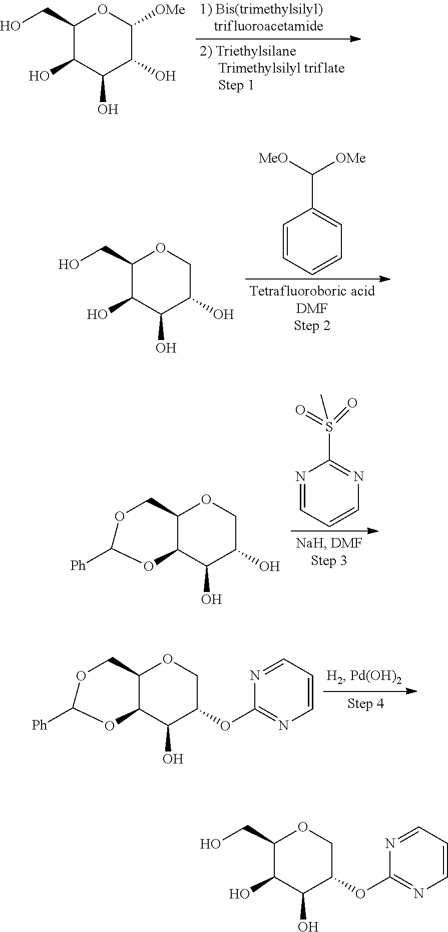

### Preparation of (2R,3R,4S,5S)-2-(hydroxymethyl)-5-(pyrimidin-2-ylthio)tetrahydro-2H-pyran-3,4-diol

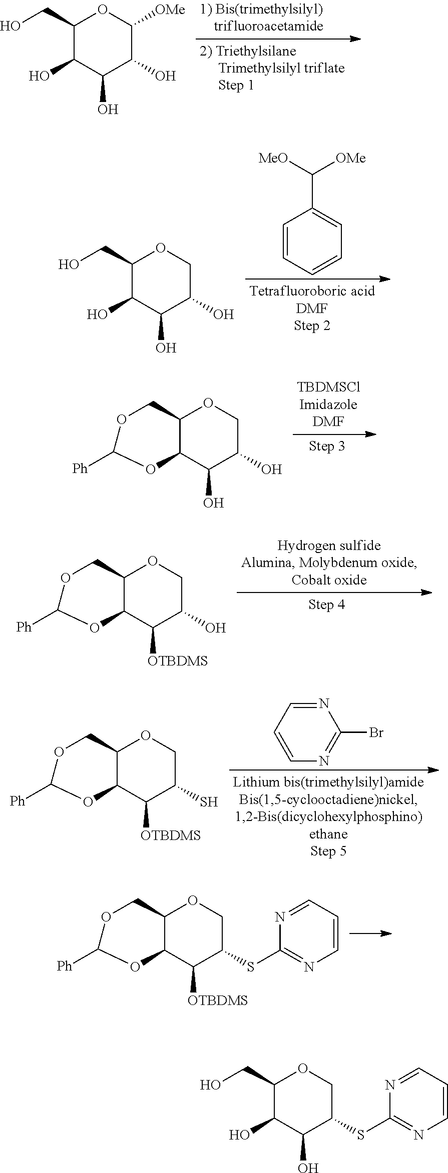

### Preparation of ((2R,3R,4R,5S)-4,5-dihydroxy-3-((4-(trifluoromethyl)pyrimidin-2-yl)amino)tetrahydro-2H-pyran-2-yl)(imino)(methyl)-16-sulfanone

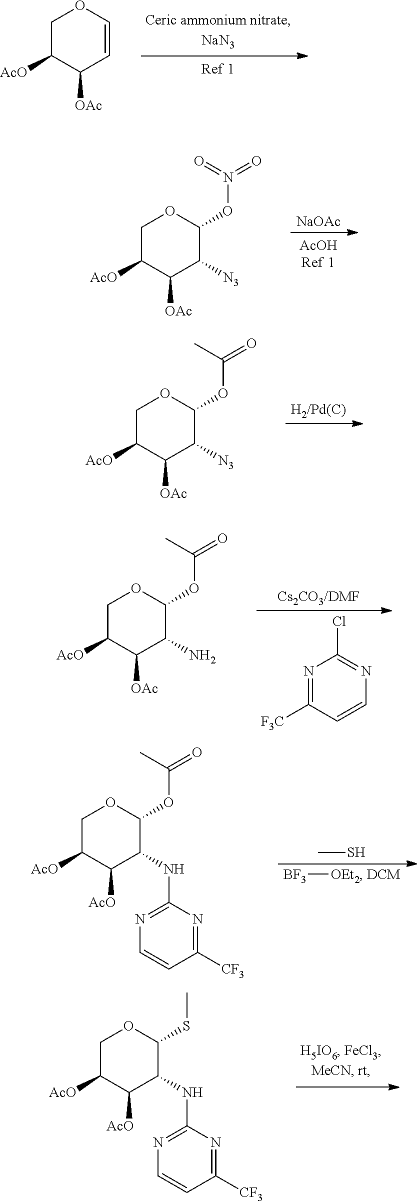

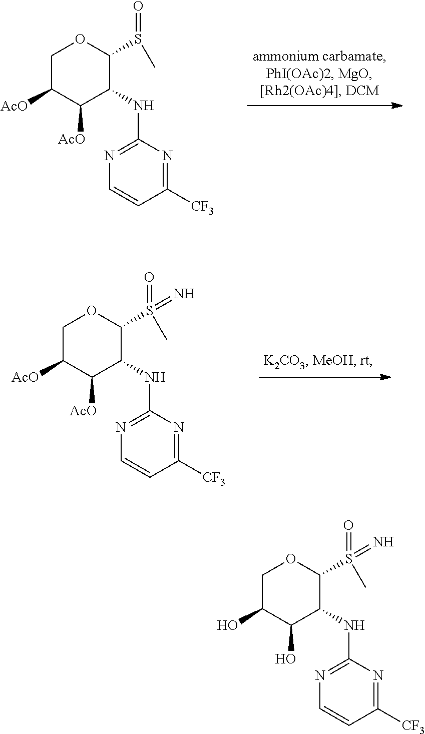 [SMILES: CC(=O)O[C@@H]1[C@@H](Nc2nccc(C(F)(F)F)n2)[C@@H](S(C)=O)OC[C@@H]1OC(C)=O ; CC(=O)O[C@@H]1[C@@H](Nc2nccc(C(F)(F)F)n2)[C@@H](S(C)(=N)=O)OC[C@@H]1OC(C)=O ; CS(=N)(=O)[C@H]1OC[C@H](O)[C@H](O)[C@H]1Nc1nccc(C(F)(F)F)n1]

### Preparation of 2-(2-(2-azidoethoxy)ethoxy)-N-(((2R,3R,4R,5S)-4,5-dihydroxy-3-((4-(trifluoromethyl)pyrimidin-2-yl)amino)tetrahydro-2H-pyran-2-yl)(methyl)(oxo)-16-sulfaneylidene)acetamide

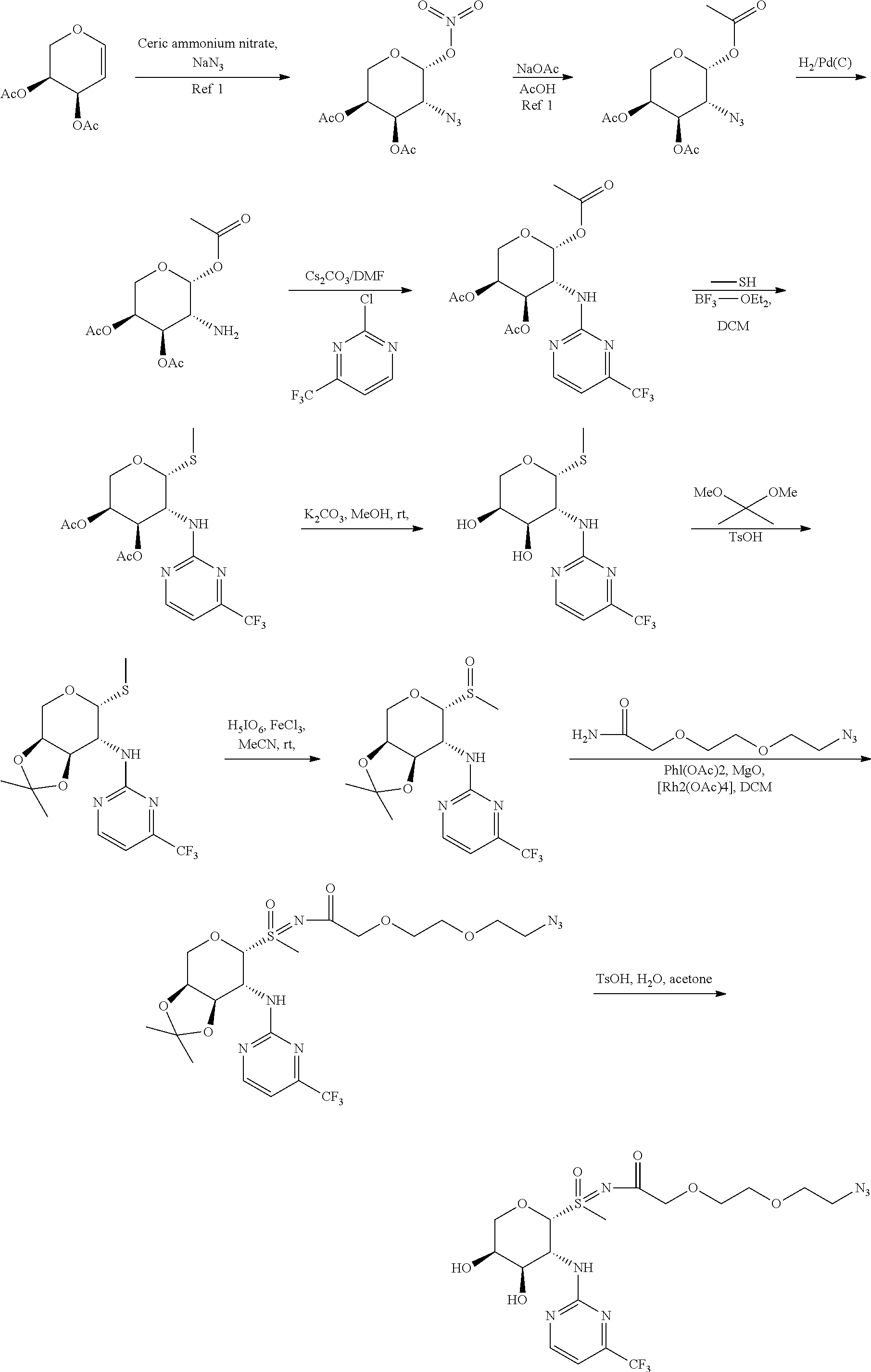

### Representative Synthesis

### Preparation of 2-(4-(2-((((2R,3R,4R,5S)-3,4-dihydroxy-5-((6-(trifluoromethyl)pyrazin-2-yl)amino)tetrahydro-2H-pyran-2-yl)methyl)amino)-2-oxoethyl)phenoxy)acetic acid (A1)

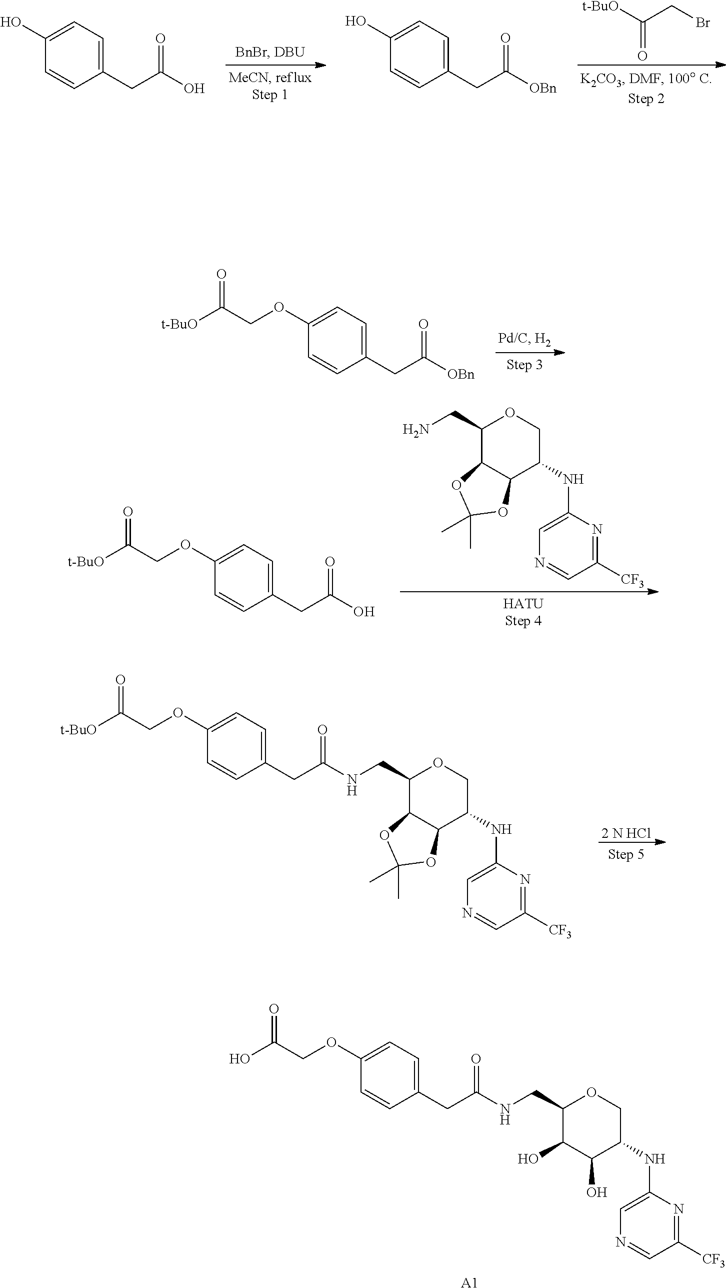

Step 1: To a solution of 2-(4-hydroxyphenyl)acetic acid (2.1 g, 13.8 mmol) in dry CH₃CN (30 mL) was added DBU (2.56 g, 16.8 mmol) and benzyl bromide (2.61 g, 15.4 mmol) at an ambient temperature. The mixture was stirred at 90° C. for 18 h. Then the reaction mixture was concentrated and purified by silica gel (PE/EA=5/1) to give benzyl 2-(4-hydroxyphenyl)acetate (2.7 g, 80% yield). LC-MS (ESI) found: 243 [M+H]⁺.
Step 2: To a solution of Benzyl 2-(4-hydroxyphenyl)acetate (2 g, 8.2 mmol) in CH₃CN (20 ml) was added tert-butyl 2-bromoacetate (2.4 g, 12.3 mmol) and K₂CO₃ (2.28 g, 16.5 mmol). The mixture was stirred at 80° C. for 18 h. The reaction mixture was filtered through a Celite pad and the solvent was concentrated. The residue was purified by silica gel (PE/EA=5/1) to give benzyl 2-(4-(2-(tert-butoxy)-2-oxoethoxy)phenyl)acetate (2.1 g, 70% yield). LC-MS (ESI) found: 374 [M+18]⁺.
Step 3: To a solution of benzyl 2-(4-(2-(tert-butoxy)-2-oxoethoxy)phenyl)acetate (2.1 g, 5.8 mmol) in MeOH (25 mL) was added Pd/C (210 mg, 10% wt., 60% wet). The mixture was stirred at an ambient temperature under a H2 balloon for 0.5 h. The mixture was filtered through a Celite pad, and the organic layer was concentrated to give crude 2-(4-(2-(tert-butoxy)-2-oxoethoxy)phenyl)acetic acid (1.5 g). LC-MS (ESI) found: 267 [M+H]⁺.
Step 4: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.0 g, 2.87 mmol) in DMF (15 mL) were added HATU (1.3 g, 3.44 mmol), TEA (869.6 mg, 8.61 mmol), and 2-(4-(2-(tert-butoxy)-2-oxoethoxy)phenyl)acetic acid (763.4 mg, 2.87 mmol). The mixture was stirred at 25° C. for 1 h. The mixture was diluted with ethyl acetate (200 mL) and washed with water (200 mL×3). The organic layer was dried over sodium sulfate, filtered, concentrated under reduced pressure to give a crude product, which was purified by column to give tert-butyl 2-(4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)phenoxy)acetate (1.2 g, 70% yield). LC-MS (ESI) found: 597 [M+H]⁺.
Step 5: A mixture of tert-butyl 2-(4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)phenoxy)acetate (20 mg, 0.046 mmol) and diluted aqueous HCl (1 mL, 2 N in H₂O) in THF (2 mL) was stirred at an ambient temperature for 30 min. Reaction mixture was concentrated. Residue was purified by flash chromatography reversed phase (C18, MeOH in water from 10% to 70%) to give compound A1 as a white solid (6.1 mg, 34% yield). LC-MS (ESI) found: 501 [M+H]⁺. ¹H NMR (400 MHz, CD₃OD): δ 8.10 (s, 1H), 7.99 (s, 1H), 4.82-4.63 (m, 1H), 4.32 (td, J=10.5, 5.1 Hz, 1H), 4.13 (dd, J=11.0, 5.2 Hz, 1H), 3.84 (d, J=2.8 Hz, 1H), 3.66-3.55 (m, 2H), 3.50-3.47 (m, 1H), 3.34 (d, J=7.9 Hz, 1H), 3.09 (t, J=10.9 Hz, 1H), 1.85-1.77 (m, 1H), 1.70-1.60 (m, 1H), 1.12-1.01 (m, 1H).

### Preparation of (2R,3R,4R,5R,6R)-6-(allyloxy)-2-(hydroxymethyl)-5-((6-(trifluoromethyl)pyrazin-2-yl)amino)tetrahydro-2H-pyran-3,4-diol (A2)

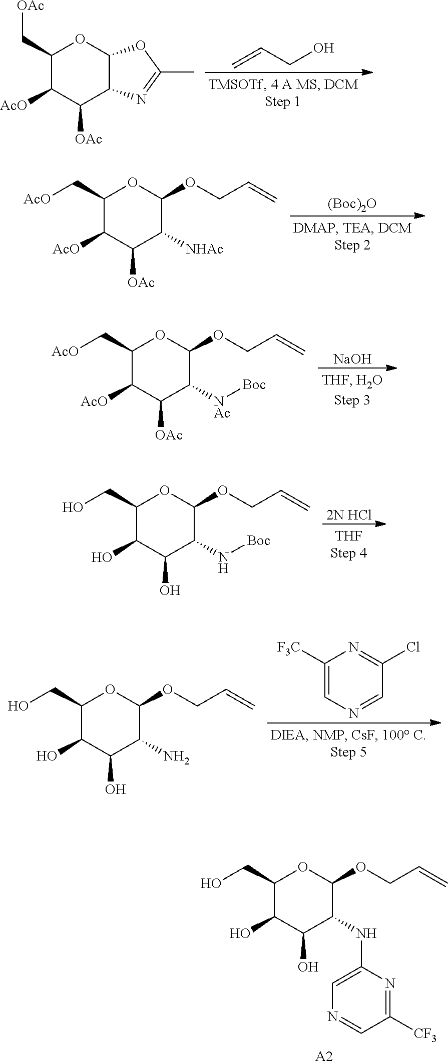

Step 1: To a solution of (3aR,5R,6R,7R,7aR)-5-(acetoxymethyl)-2-methyl-3a,6,7,7a-tetrahydro-5H-pyrano[3,2-d]oxazole-6,7-diyl diacetate (1.0 g, 3.04 mmol) in DCM (20 mL) was added prop-2-en-1-ol (529 mg, 9.12 mmol) and TMSOTf (675 mg, 3.04 mmol). The mixture was stirred at room temperature under N₂ for 2 h. The mixture was diluted with ethyl acetate (10 mL) and washed with water (10 mL×3). The organic layer was concentrated under reduced pressure to give a crude product, which was purified by column to give (2R,3R,4R,5R,6R)-5-acetamido-2-(acetoxymethyl)-6-(allyloxy)tetrahydro-2H-pyran-3,4-diyl diacetate (500 mg, 42% yield). LC-MS (ESI) found: 388 [M+H]⁺.
Step 2: To a solution of (2R,3R,4R,5R,6R)-5-acetamido-2-(acetoxymethyl)-6-(allyloxy)tetrahydro-2H-pyran-3,4-diyl diacetate (500 mg, 1.29 mmol) in DCM (20 mL) was added Boc₂O (1.4 g, 6.45 mmol), DMAP (53.5 mg, 0.65 mmol) and TEA (391 mg, 3.87 mmol). The mixture was stirred at the room temperature for 16 h. The mixture was concentrated under reduced pressure to give a crude product, which was purified by column to give (2R,3R,4R,5R,6R)-2-(acetoxymethyl)-6-(allyloxy)-5-(N-(tert-butoxycarbonyl)acetamido)tetrahydro-2H-pyran-3,4-diyl diacetate (400 mg, 64% yield). LC-MS (ESI) found: 487 [M+H]⁺.
Step 3: To a solution of (2R,3R,4R,5R,6R)-2-(acetoxymethyl)-6-(allyloxy)-5-(N-(tert-butoxycarbonyl)acetamido)tetrahydro-2H-pyran-3,4-diyl diacetate (400 mg, 0.821 mmol) in THE (10 mL) and H₂O (5 mL) was added NaOH (33 mg, 0.821 mmol). The mixture was stirred at 80° C. for 16 h. The mixture was concentrated under reduced pressure to give a crude product, which was purified by column to give tert-butyl ((2R,3R,4R,5R,6R)-2-(allyloxy)-4,5-dihydroxy-6-(hydroxymethyl)tetrahydro-2H-pyran-3-yl)carbamate (150 mg, 57% yield). LC-MS (ESI) found: 320 [M+H]⁺.
Step 4: To a solution of tert-butyl ((2R,3R,4R,5R,6R)-2-(allyloxy)-4,5-dihydroxy-6-(hydroxymethyl)tetrahydro-2H-pyran-3-yl)carbamate (150 mg, 0.47 mmol) in THF (10 mL) was added HCl solution (1 mL, 2 N in H₂O). The mixture was stirred at room temperature for 1 h. The mixture was concentrated under reduced pressure to give crude (2R,3R,4R,5R,6R)-6-(allyloxy) 5-amino-2-(hydroxymethyl)tetrahydro-2H-pyran-3,4-diol (100 mg crude). LC-MS (ESI) found: 220 [M+H]⁺.
Step 5: To a solution of (2R,3R,4R,5R,6R)-6-(allyloxy)-5-amino-2-(hydroxymethyl)tetrahydro-2H-pyran-3,4-diol (100 mg, 0.456 mol) in NMP (2 mL) was added 2-chloro-6-(trifluoromethyl)pyrazine (83 mg, 0.456 mmol), CsF (35 mg, 0.228 mmol) and DIEA (90 mg, 0.684 mmol). The mixture was stirred at 100° C. for 16 h. The mixture was concentrated under reduced pressure to give a crude product, which was purified by column to give compound A2 (11 mg, 7% yield). ¹H NMR (400 MHz, CD₃OD): δ 8.15 (s, 1H), 7.99 (s, 1H), 5.74 (ddd, J=16.1, 10.4, 5.2 Hz, 1H), 5.07 (dd, J=17.3, 1.6 Hz, 1H), 4.98 (d, J=10.5 Hz, 1H), 4.54 (d, J=8.3 Hz, 1H), 4.54 (d, J=8.3 Hz, 1H), 4.38-4.29 (m, 1H), 4.21-4.10 (m, 1H), 4.03 (dd, J=13.4, 5.6 Hz, 1H), 3.90 (d, J=2.7 Hz, 1H), 3.84-3.73 (m, 3H), 3.55 (t, J=6.0 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1,2,3,4-tetrahydroisoquinoline-6-carboxamide (A3)

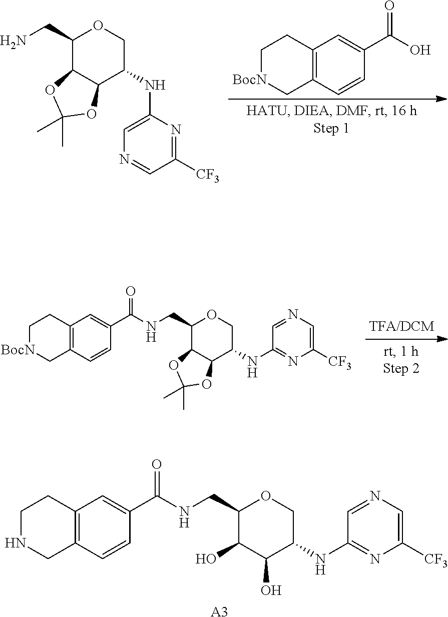 [SMILES: CC1(C)O[C@@H]2[C@H](O1)[C@@H](Nc1cncc(C(F)(F)F)n1)CO[C@@H]2CN ; CC(C)(C)OC(=O)N1CCc2cc(C(=O)O)ccc2C1 ; CC(C)(C)OC(=O)N1CCc2cc(C(=O)NC[C@H]3OC[C@H](Nc4cncc(C(F)(F)F)n4)[C@H]4OC(C)(C)O[C@H]43)ccc2C1 ; O=C(NC[C@H]1OC[C@H](Nc2cncc(C(F)(F)F)n2)[C@@H](O)[C@H]1O)c1ccc2c(c1)CCNC2 ; "A3"]

Step 1: To a mixture of 2-(tert-butoxycarbonyl)-1,2,3,4-tetrahydroisoquinoline-6-carboxylic acid (40 mg, 0.14 mmol) and HATU (60 mg, 0.15 mmol) in DMF (5 mL) was added DIEA (37 mg, 0.28 mmol) at 0° C. The reaction mixture was stirred at 0° C. for 0.5 h, and then N-((3aS,4R,7S,7aR)-4-(aminomethyl)-2,2-dimethyltetrahydro-4H-[1,3]dioxolo[4,5-c]pyran-7-yl) 6-(trifluoromethyl)pyrazin-2-amine (50 mg, 0.14 mmol) was added into the solution and the reaction was stirred overnight at an ambient temperature. After completion, the mixture was quenched with H₂O (5 mL) and extracted with EA (3*10 mL). The combined organic layers were washed with brine (3*10 mL), dried over anhydrous Na₂SO₄ and filtered. The filtrate was concentrated and purified by pre-TLC (DCM/MeOH=10:1) to give tert-butyl 6-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-3,4-dihydroisoquinoline-2(1H)-carboxylate (70 mg, 80% yield) as yellow gel. LC-MS (ESI) found: 608 [M+H]⁺.
Step 2: To a mixture of tert-butyl 6-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-3,4-dihydroisoquinoline-2(1H)-carboxylate (70 mg, 0.11 mmol) in DCM (5 mL) was added TFA (1 mL) at an ambient temperature and stirred for 1 h. The mixture was concentrated and the residue was purified by C18 column to give compound A3 (10 mg, 20% yield) as brown solid. LC-MS (ESI) found: 468 [M+H]⁺. ¹H NMR (400 MHz, DMSO-d₆): δ 8.40 (s, 1H), 8.21 (s, 1H), 8.07 (s, 1H), 7.69-7.53 (m, 3H), 7.09 (d, J=8.0 Hz, 1H), 4.84 (s, 2H), 4.08 (s, 1H), 3.96-3.84 (m, 3H), 3.72 (s, 1H), 3.52 (s, 3H), 3.42 (d, J=6.4 Hz, 2H), 2.97 (d, J=5.9 Hz, 3H), 2.73 (s, 2H).

### Preparation of N-(((2R,3R,4R,5S)-3,4-dihydroxy-5-((6-(trifluoromethyl) pyrazin-2-yl)amino)tetrahydro-2H-pyran-2-yl)methyl)pyrimidine-2-carboxamide (A4)

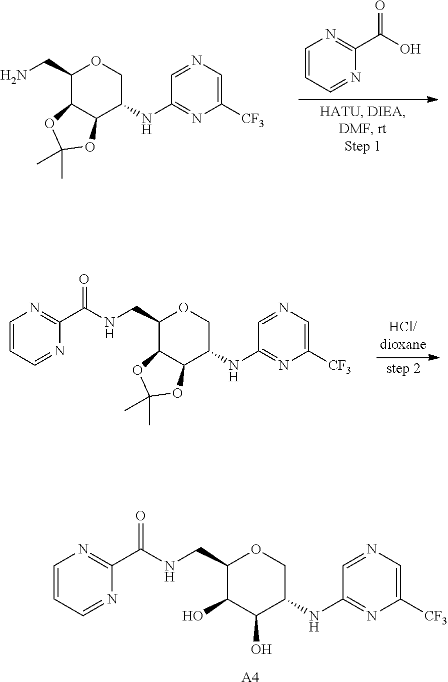

Step 1: A solution of pyrimidine-2-carboxylic acid (18 mg, 0.14 mmol), DIEA (0.1 mL, 0.57 mmol) and HATU (80 mg, 0.21 mmol) in DMF (5 mL) was stirred at an ambient temperature for 30 min N-((3aR,4R,7S,7aR)-4-(13-amino-2,5,8,11-tetraoxatridecyl)-2,2-dimethyltetrahydro-4H-[1,3]dioxolo[4,5-c]pyran-7-yl)-6-(trifluoromethyl)pyrazin-2-amine (50 mg, 0.14 mmol) was added at an ambient temperature. The reaction was stirred overnight. The resulting mixture was concentrated in vacuo. The crude product was purified by flash chromatography (silica gel, 0˜10% MeOH in DCM) to give N-(((3aS,4R,7S,7aR)-2,2-dimethyl-7-((6-(trifluoromethyl) pyrazin-2-yl) amino) tetrahydro-4H-[1,3]dioxolo[4,5-c]pyran-4-yl)methyl)pyrimidine-2-carboxamide (40 mg, 63% yield) as a colorless oil. LC-MS (ESI) found: 455 [M+H]⁺.
Step 2: A solution of N-(((3aS,4R,7S,7aR)-2,2-dimethyl-7-((6-(trifluoromethyl) pyrazin-2-yl) amino) tetrahydro-4H-[1,3]dioxolo[4,5-c]pyran-4-yl)methyl)pyrimidine-2-carboxamide (40 mg, 0.08 mmol) in HCl/dioxane (5 mL, 4 N) was stirred at an ambient temperature for 2 h. The resulting mixture was purified by C18 column to give compound A4 as a white solid. LC-MS (ESI) found: 415 [M+H]⁺. ¹H NMR (400 MHz, CD₃OD): δ 8.94 (d, J=4.8 Hz, 1H), 8.11 (s, 1H), 7.99 (s, 1H) 7.63 (t, J=4.8 Hz, 1H), 4.63 (s, 1H), 4.36 (td, J=10.5, 5.1 Hz, 1H), 4.15 (dd, J=11.0, 5.1 Hz, 1H), 3.94 (d, J=2.3 Hz, 1H), 3.79 (dd, J=13.1, 4.1 Hz, 1H), 3.73-3.56 (m, 2H), 3.13 (t, J=10.8 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1H-benzo[d]imidazole-5-carboxamide (A6)

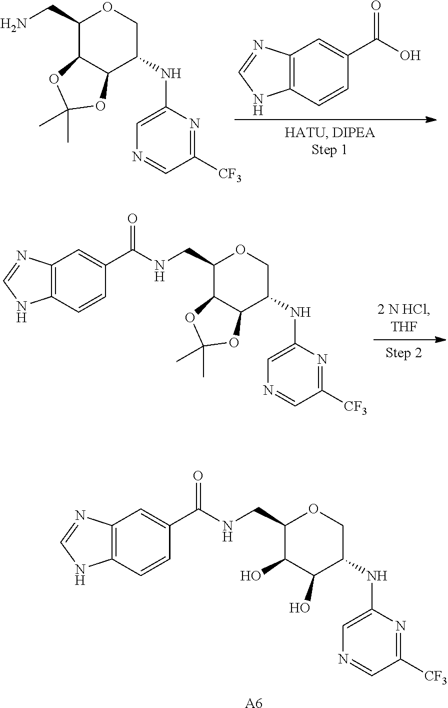 [SMILES: CC1(C)O[C@@H]2[C@H](O1)[C@@H](Nc1cncc(C(F)(F)F)n1)CO[C@@H]2CN ; O=C(O)c1ccc2[nH]cnc2c1 ; CC1(C)O[C@@H]2[C@H](O1)[C@@H](Nc1cncc(C(F)(F)F)n1)CO[C@@H]2CNC(=O)c1ccc2[nH]cnc2c1 ; O=C(NC[C@H]1OC[C@H](Nc2cncc(C(F)(F)F)n2)[C@@H](O)[C@H]1O)c1ccc2[nH]cnc2c1 ; "A6"]

Step 1: To a solution of 1H-benzo[d]imidazole-5-carboxylic acid (28 mg, 0.17 mmol) in DMF (3 mL) were added HATU (66 mg, 0.17 mmol) and DIPEA (597 mg, 4.63 mmol) at an ambient temperature. After stirring at an ambient temperature for 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, then concentrated. The residue was purified by flash to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-benzo[d]imidazole-5-carboxamide (60 mg, 85% yield) as brown oil. LC-MS (ESI) found: 493 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-benzo[d]imidazole-5-carboxamide (60 mg, 0.12 mmol) in THF (3 mL) was added HCl (0.5 mL, 2 N in H₂O) at an ambient temperature. The mixture was stirred at an ambient temperature overnight, then concentrated. The residue was purified by flash to give compound A6 (25 mg, 54% yield) as white solid. LC-MS (ESI) found: 453 [M+H]⁺. ¹H NMR (400 MHz, CD₃OD): δ 8.31 (s, 1H) 8.17 (s, 1H), 8.11 (s, 1H), 8.00 (s, 1H), 7.80 (d, J=8.5 Hz, 1H), 7.67 (d, J=8.5 Hz, 1H), 4.37 (td, J=10.7, 5.2 Hz, 1H), 4.15 (dd, J=11.0, 5.0 Hz, 1H), 3.93 (d, J=2.7 Hz, 1H), 3.76 (dd, J=13.4, 5.0 Hz, 1H), 3.68 (dd, J=10.1, 3.2 Hz, 2H), 3.55 (dd, J=13.3, 7.4 Hz, 1H), 3.13 (t, J=10.8 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-6-phenylnicotinamide (A7)

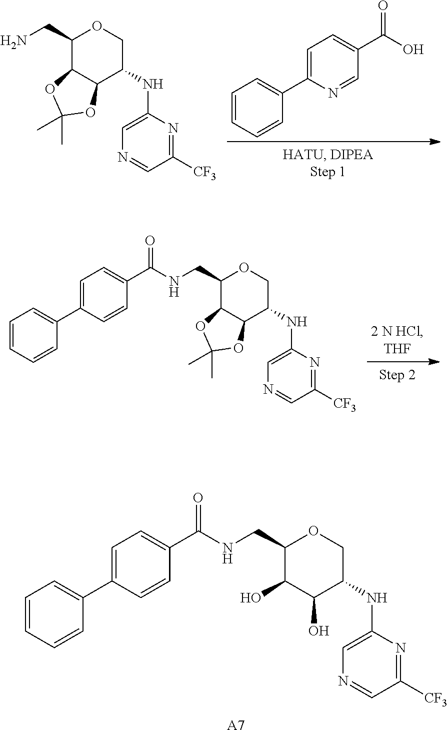 [SMILES: CC1(C)O[C@@H]2[C@H](O1)[C@@H](Nc1cncc(C(F)(F)F)n1)CO[C@@H]2CN ; O=C(O)c1ccc(-c2ccccc2)nc1 ; CC1(C)O[C@@H]2[C@H](O1)[C@@H](Nc1cncc(C(F)(F)F)n1)CO[C@@H]2CNC(=O)c1ccc(-c2ccccc2)cc1 ; O=C(NC[C@H]1OC[C@H](Nc2cncc(C(F)(F)F)n2)[C@@H](O)[C@H]1O)c1ccc(-c2ccccc2)cc1 ; "A7"]

Step 1: To a solution of 6-phenylnicotinic acid (34 mg, 0.17 mmol) in DMF (3 mL) were added HATU (66 mg, 0.17 mmol) and DIPEA (597 mg, 4.63 mmol) at an ambient temperature. After stirring at an ambient temperature for 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, then concentrated. The residue was purified by flash to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-6-phenylnicotinamide (50 mg, 66% yield) as brown oil. LC-MS (ESI) found: 530 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-6-phenylnicotinamide (50 mg, 0.09 mmol) in THF (3 mL) was added HCl (0.5 mL, 2 N in H₂O) at an ambient temperature. The mixture was stirred at an ambient temperature overnight, then concentrated. The residue was purified by flash to give compound A7 (15 mg, 33%) as white solid. LC-MS (ESI) found: 489 [M+H]⁺. ¹H NMR (400 MHz, CD₃OD): δ 9.06 (d, J=1.7 Hz, 1H), 8.29 (dd, J=8.4, 2.2 Hz, 1H) 8.11 (s, 1H), 8.06-8.02 (m, 2H), 8.01-7.94 (m, 2H), 7.54-7.43 (m, 3H), 4.37 (td, J=10.5, 5.2 Hz, 1H), 4.16 (dd, J=11.1, 5.1 Hz, 1H), 3.94 (d, J=2.9 Hz, 1H), 3.76 (dd, J=13.4, 4.5 Hz, 1H) 3.73-3.66 (m, 2H), 3.57 (dd, J=13.4, 7.7 Hz, 1H), 3.14 (t, J=10.9 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)quinoline-3-carboxamide (A8)

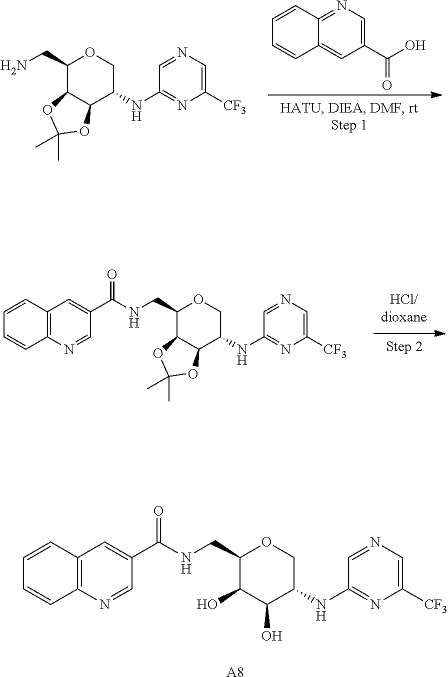 [SMILES: CC1(C)O[C@@H]2[C@H](O1)[C@@H](Nc1cncc(C(F)(F)F)n1)CO[C@@H]2CN ; O=C(O)c1cnc2ccccc2c1 ; CC1(C)O[C@@H]2[C@H](O1)[C@@H](Nc1cncc(C(F)(F)F)n1)CO[C@@H]2CNC(=O)c1cnc2ccccc2c1 ; O=C(NC[C@H]1OC[C@H](Nc2cncc(C(F)(F)F)n2)[C@@H](O)[C@H]1O)c1cnc2ccccc2c1 ; "A8"]

Step 1: A solution of quinoline-3-carboxylic acid (27.34 mg, 0.16 mmol), DIEA (56 mg, 0.43 mmol) and HATU (60 mg, 0.16 mmol) in DMF (3 mL) was stirred at an ambient temperature for min. N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added at an ambient temperature. The reaction was stirred overnight. The resulting mixture was concentrated in vacuo. The crude product was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinoline-3-carboxamide (60 mg, 83% yield) as a colorless oil. LC-MS (ESI) found: 504 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinoline-3-carboxamide (60 mg, 0.12 mmol) in HCl/dioxane (5 mL, 4 N) was stirred at an ambient temperature for 2 h. The resulting mixture was purified by C18 column to give compound A8 (8.1 mg, 15% yield) as a white solid. LC-MS (ESI) found: 464 [M+H]⁺. ¹H NMR (400 MHz, CD₃OD): δ 9.26 (d, J=1.5 Hz, 1H), 8.82 (s, 1H), 8.13-8.05 (m, 3H), 8.00 (s, 1H), 7.89 (t, J=7.7 Hz, 1H), 7.71 (t, J=7.5 Hz, 1H), 4.37 (td, J=10.4, 4.9 Hz, 1H), 4.17 (dd, J=11.1, 5.1 Hz, 1H), 3.96 (d, J=2.8 Hz, 1H), 3.81 (dd, J=13.3, 4.3 Hz, 1H), 3.76-3.66 (m, 2H), 3.61 (dd, J=13.4, 7.8 Hz, 1H), 3.15 (t, J=10.9 Hz, 1H).

### Preparation of (2R,3R,4R,5S)-2-((4-benzylpiperazin-1-yl)methyl)-5-((6-(trifluoromethyl)pyrazin-2-yl)amino)tetrahydro-2H-pyran-3,4-diol (A9)

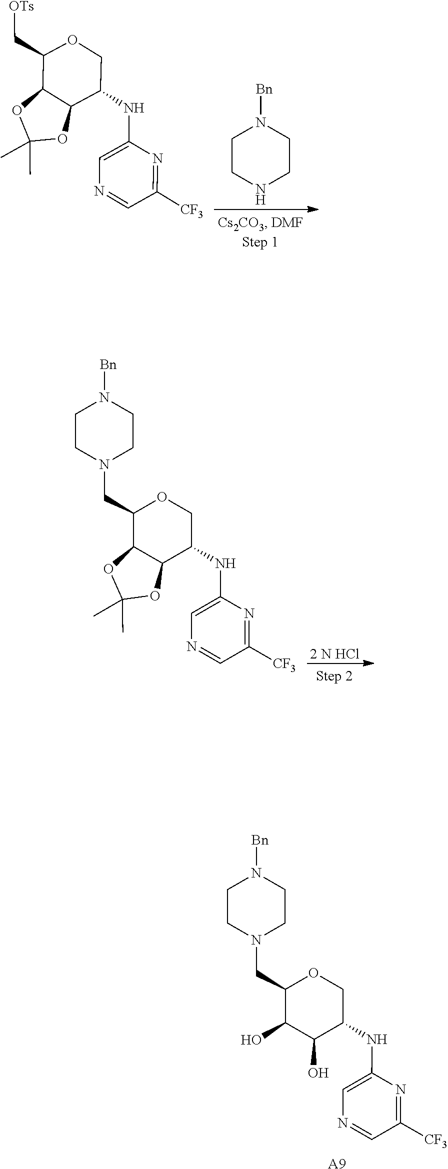

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (1 g, 1.98 mmol) in DMF (15 mL) was added Cs₂CO₃ (1.3 g, 3.97 mmol) and 1-benzylpiperazine (0.4 g, 2.27 mmol) at an ambient temperature. Then the reaction was stirred at 100° C. for 18 h. Then the reaction was added water and extracted with EA. The organic layer was concentrated and purified by reverse phase with MeOH/H₂O to give N-((3aS,4R,7S,7aR)-4-((4-benzylpiperazin-1-yl)methyl)-2,2-dimethyltetrahydro-4H-[1,3]dioxolo[4,5-c]pyran-7-yl)-6-(trifluoromethyl)pyrazin-2-amine (120 mg, 12% yield) as yellow oil. LC-MS (ESI) found: 508 [M+H]⁺.
Step 2: To a solution of N-((3aS,4R,7S,7aR)-4-((4-benzylpiperazin-1-yl)methyl)-2,2-dimethyltetrahydro-4H-[1,3]dioxolo[4,5-c]pyran-7-yl)-6-(trifluoromethyl)pyrazin-2-amine (120 mg, 0.24 mmol) in THF (5 mL) was added HCl solution (1 mL, 2 N in H₂O) at an ambient temperature. The reaction was stirred at an ambient temperature for 1 h. The reaction was concentrated and purified by reverse phase with MeOH/H₂O to give compound A9 (60 mg, 54% yield) as white solid. LC-MS (ESI) found: 468 [M+H]⁺. ¹H NMR (400 MHz, CD₃OD): δ 1H NMR (400 MHz, CD₃OD) δ 8.13 (s, 1H), 8.01 (s, 1H), 7.32-7.51 (m, 5H), 4.28-4.41 (m, 1H), 4.17 (dd, J=5.2, 11.2 Hz, 1H), 3.80-4.00 (m, 4H), 3.71 (dd, J=2.8, 11.8 Hz, 1H), 3.33-3.58 (m, 3H), 2.70-3.287 (m, 8H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-[1,1′-biphenyl]-4-carboxamide (A10)

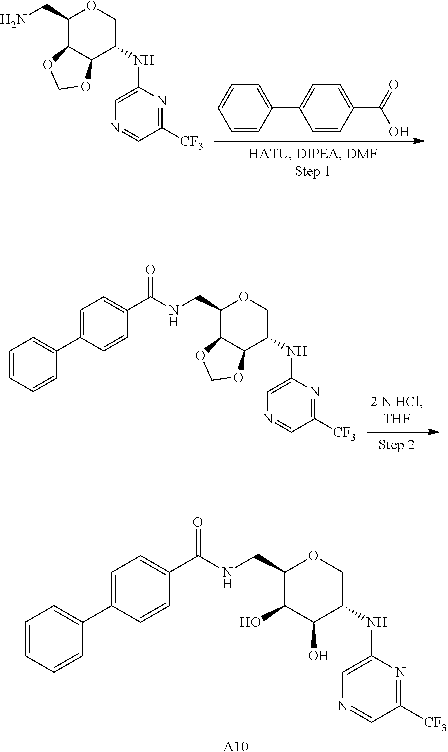 [SMILES: NC[C@H]1OC[C@H](Nc2cncc(C(F)(F)F)n2)[C@H]2OCO[C@H]21 ; O=C(O)c1ccc(-c2ccccc2)cc1 ; O=C(NC[C@H]1OC[C@H](Nc2cncc(C(F)(F)F)n2)[C@H]2OCO[C@H]21)c1ccc(-c2ccccc2)cc1 ; O=C(NC[C@H]1OC[C@H](Nc2cncc(C(F)(F)F)n2)[C@@H](O)[C@H]1O)c1ccc(-c2ccccc2)cc1 ; "A10"]

Step 1: To a solution of [1,1′-biphenyl]-4-carboxylic acid (31 mg, 0.16 mmol) in DMF (2 mL) were added HATU (66 mg, 0.17 mmol) and DIPEA (37 mg, 0.29 mmol) at an ambient temperature. After stirring for 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, and then concentrated. The residue was purified by flash chromatography to give N-(((2R,3R,4R,5S)-3,4-dihydroxy-5-((6-(trifluoromethyl)pyrazin-2-yl)amino)tetrahydro-2H-pyran-2-yl)methyl)-[1,1′-biphenyl]-4-carboxamide (70 mg, 92% yield) as pale yellow oil. LC-MS (ESI) found: 529 [M+H]⁺.
Step 2: To a solution of N-(((2R,3R,4R,5S)-3,4-dihydroxy-5-((6-(trifluoromethyl)pyrazin-2-yl)amino)tetrahydro-2H-pyran-2-yl)methyl)-[1,1′-biphenyl]-4-carboxamide (70 mg, 0.13 mmol) in THF (3 mL) was added HCl (0.5 mL, 2 N in H₂O) at an ambient temperature. The mixture was stirred at an ambient temperature overnight, then concentrated. The residue was purified by C18 column to give compound A10 (26 mg, 40% yield) as white solid. LC-MS (ESI) found: 489 [M+H]⁺. ¹H NMR (400 MHz, CD₃OD): δ 8.11 (s, 1H), 8.00 (s, 1H), 7.92 (d, J=8.4 Hz, 2H) 7.73 (d, J=8.4 Hz, 2H), 7.67 (d, J=7.4 Hz, 2H), 7.46 (t, J=7.5 Hz, 2H), 7.39 (d, J=7.2 Hz, 1H), 4.36 (d, J=5.0 Hz, 1H), 4.15 (dd, J=10.9, 5.1 Hz, 1H), 3.93 (s, 1H), 3.75 (d, J=13.4 Hz, 1H), 3.68 (d, J=11.1 Hz, 2H), 3.54 (dd, J=13.1, 7.4 Hz, 1H), 3.15 (d, J=10.6 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)benzofuran-5-carboxamide (All)

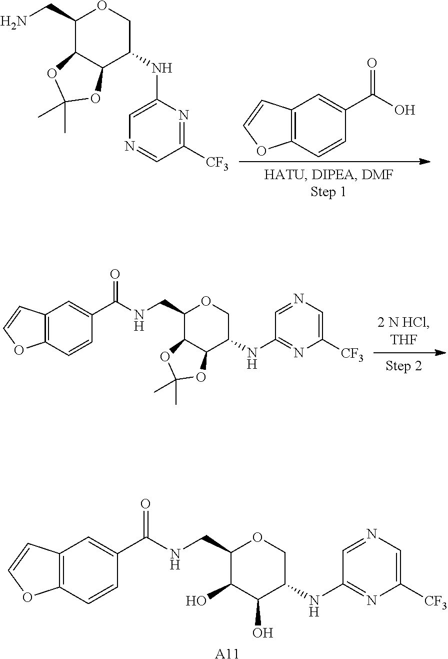

Step 1: To a solution of benzofuran-5-carboxylic acid (26 mg, 0.16 mmol) in DMF (2 mL) were added HATU (66 mg, 0.17 mmol) and DIPEA (37 mg, 0.29 mmol) at an ambient temperature. After stirring 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, then concentrated. The residue was purified by flash chromatography flash to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)benzofuran-5-carboxamide (61 mg, 86% yield) as pale yellow oil. LC-MS (ESI) found: 493 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)benzofuran-5-carboxamide (61 mg, 0.12 mmol) in THF (3 mL) was added HCl solution (0.5 mL, 2 N in H₂O) at an ambient temperature. The mixture was stirred at an ambient temperature overnight, then concentrated. The residue was purified by C18 column to give compound A11 (25 mg, 45% yield) as white solid. LC-MS (ESI) found: 453 [M+H]⁺. ¹H NMR (400 MHz, CD₃OD): δ 8.13 (d, J=15.9 Hz, 2H), 8.00 (s, 1H), 7.86-7.78 (m, 2H), 7.57 (d, J=8.6 Hz, 1H), 6.94 (s, 1H), 4.36 (td, J=10.5, 5.2 Hz, 1H), 4.15 (dd, J=11.0, 5.1 Hz, 1H), 3.93 (d, J=2.8 Hz, 1H), 3.75 (dd, J=13.4, 4.9 Hz, 1H), 3.68 (dd, J=6.9, 4.2 Hz, 2H), 3.54 (dd, J=13.4, 7.5 Hz, 1H), 3.13 (t, J=10.8 Hz, 1H).

### Preparation of 2-((3R,5R,7R)-adamantan-1-yl)-N-(((2R,3R,4R,5S)-3,4-dihydroxy-5-((6-(trifluoromethyl)pyrazin-2-yl)amino)tetrahydro-2H-pyran-2-yl)methyl)acetamide (A12)

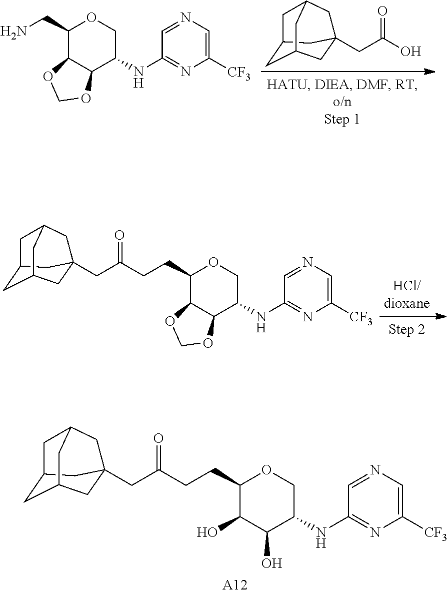

Step 1: A solution of 2-((3r,5r,7r)-adamantan-1-yl)acetic acid (31 mg, 0.16 mmol), DIEA (56 mg, 0.43 mmol) and HATU (60 mg, 0.16 mmol) in DMF (3 mL) was stirred at an ambient temperature for 30 min. N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added at an ambient temperature. The reaction was stirred overnight. The resulting mixture was concentrated in vacuo. The crude product was purified by flash chromatography (silica gel, 0˜10% MeOH in DCM) to give 2-((3R,5R,7R)-adamantan-1-yl)-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)acetamide (70 mg, 93% yield) as a colorless oil. LC-MS (ESI) found: 525 [M+H]⁺.
Step 2: To a solution of 2-((3R,5R,7R)-adamantan-1-yl)-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)acetamide (70 mg, 0.13 mmol) in HCl/dioxane (5 mL, 4 N) was stirred at an ambient temperature for 2 h. The resulting mixture was purified by C18 column to give Compound A12 (23 mg, 35% yield) as a white solid. LC-MS (ESI) found: 485 [M+H]⁺. ¹H NMR (400 MHz, CD₃OD): δ 8.10 (s, 1H), 8.00 (s, 1H), 4.31 (s, 1H), 4.11 (dd, J=11.0, 5.3 Hz, 1H), 3.85 (s, 1H) 3.64 (d, J=10.6 Hz, 1H), 3.51 (d, J=10.4 Hz, 2H), 3.27-3.23 (m, 1H), 3.09 (dd, J=21.4, 10.5 Hz, 1H), 1.95 (s, 5H), 1.74 (d, J=12.5 Hz, 3H), 1.66 (d, J=16.1 Hz, 9H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1H-benzo[d]imidazole-2-carboxamide (A13)

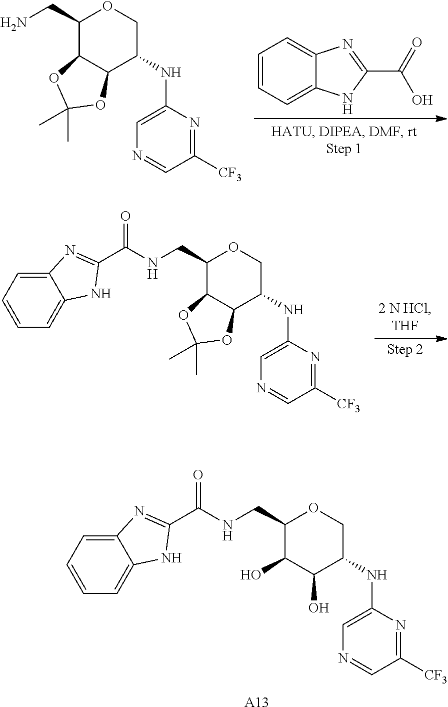 [SMILES: CC1(C)O[C@@H]2[C@H](O1)[C@@H](Nc1cncc(C(F)(F)F)n1)CO[C@@H]2CN ; O=C(O)c1nc2ccccc2[nH]1 ; CC1(C)O[C@@H]2[C@H](O1)[C@@H](Nc1cncc(C(F)(F)F)n1)CO[C@@H]2CNC(=O)c1nc2ccccc2[nH]1 ; O=C(NC[C@H]1OC[C@H](Nc2cncc(C(F)(F)F)n2)[C@@H](O)[C@H]1O)c1nc2ccccc2[nH]1 ; "A13"]

Step 1: To a solution of 1H-benzo[d]imidazole-2-carboxylic acid (50 mg, 0.30 mmol) in DMF (5 mL) were added HATU (140 mg, 0.37 mmol) and DIEA (119 mg, 0.92 mmol). The mixture was stirred at 0° C. for 0.5 h. Then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (102 mg, 0.29 mmol) in DMF (0.5 mL) was added. The mixture was stirred at an ambient temperature for 1.5 h. The reaction mixture was purified by C18 column to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-benzo[d]imidazole-2-carboxamide as white solid (30 mg, 20% yield). LC-MS (ESI) found: 493 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-benzo[d]imidazole-2-carboxamide (30 mg, 0.06 mmol) in THF (2 mL) was added HCl solution (0.5 mL, 2 N in H₂O). The mixture was stirred at an ambient temperature for 1 h. The reaction mixture was purified by C18 column to give compound A13 (2 mg, 7% yield). LC-MS (ESI) found: 453 [M+H]⁺. ¹H NMR (400 MHz, CD₃OD): δ 8.11 (s, 1H), 8.00 (s, 1H), 7.72 (s, 1H), 7.59 (s, 1H), 7.33 (s, 2H), 4.37 (td, J=5.6, 10.8 Hz, 1H), 4.17 (dd, J=5.2, 11.2 Hz, 1H), 3.95 (d, J=3.2 Hz, 1H), 3.62-3.79 (m, 4H), 3.16 (t, J=11.2 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1H-indole-2-carboxamide (A14)

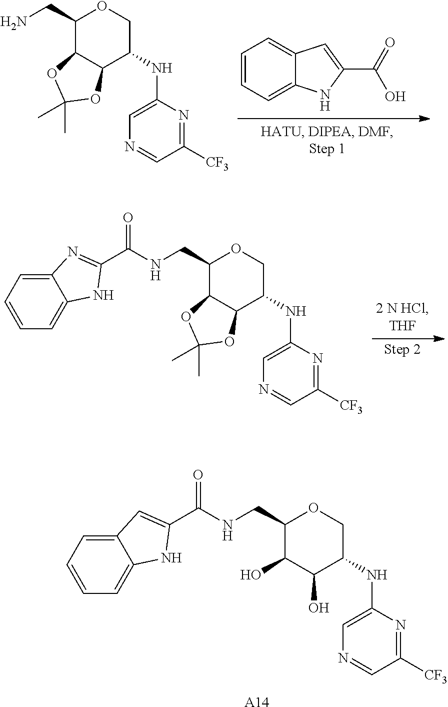 [SMILES: CC1(C)O[C@@H]2[C@H](O1)[C@@H](Nc1cncc(C(F)(F)F)n1)CO[C@@H]2CN ; O=C(O)c1cc2ccccc2[nH]1 ; CC1(C)O[C@@H]2[C@H](O1)[C@@H](Nc1cncc(C(F)(F)F)n1)CO[C@@H]2CNC(=O)c1nc2ccccc2[nH]1 ; O=C(NC[C@H]1OC[C@H](Nc2cncc(C(F)(F)F)n2)[C@@H](O)[C@H]1O)c1cc2ccccc2[nH]1 ; "A14"]

Step 1: To a solution of 1H-indole-2-carboxylic acid (50 mg, 0.31 mmol) in DMF (5 mL) were added HATU (141 mg, 0.37 mmol) and DIEA (120 mg, 0.93 mmol). The mixture was stirred at 0° C. for 0.5 h. Then a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (102 mg, 0.29 mmol) in DMF (0.5 mL) was added. The mixture was stirred at an ambient temperature for 1.5 h. The reaction mixture was purified by C18 column to give N-(((3aS,4R,7S,7aR)-2,2-dimethyl-7-((6-(trifluoromethyl)pyrazin-2-yl)amino)tetrahydro-4H-[1,3]dioxolo[4, 5-c]pyran-4-yl)methyl)-1H-indole-2-carboxamide as white solid (15 mg, 10% yield). LC-MS (ESI) found: 492 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-indole-2-carboxamide (30 mg, 0.03 mmol) in THF (2 ml) was added HCl solution (0.5 mL, 2 N in H₂O). The mixture was stirred at an ambient temperature for 1 h. The reaction mixture was purified by C 18 column to give compound A14 as white solid (1.3 mg, 10% yield). LC-MS (ESI) found: 452 [M+1]⁺. ¹H NMR (400 MHz, CD₃OD): δ 8.11 (s, 1H), 8.00 (s, 1H), 7.60 (d, J=7.6 Hz, 1H), 7.43 (d, J=7.6 Hz, 1H), 7.21 (t, J=7.6 Hz, 1H), 6.99-7.10 (m, 2H), 4.36 (td, J=5.2, 10.8 Hz, 1H), 4.15 (dd, J=5.2, 10.8 Hz, 1H), 3.93 (d, J=2.8 Hz, 1H), 3.65-3.77 (m, 3H), 3.52 (dd, J=7.6, 13.6 Hz, 1H), 3.13 (t, J=10.8 Hz, 1H).

### Preparation of (1S,3S)—N-(((2R,3R,4R,5S)-3,4-dihydroxy-5-((6-(trifluoromethyl)pyrazin-2-yl)amino)tetrahydro-2H-pyran-2-yl)methyl)adamantane-1-carboxamide (A15)

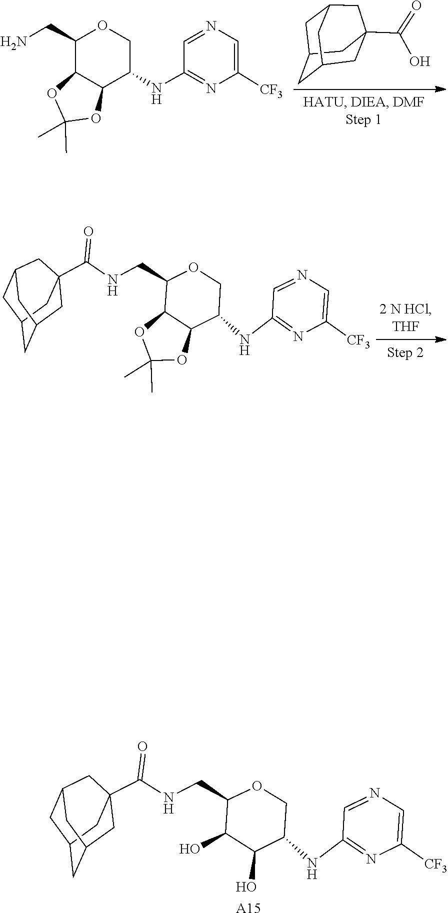

Step 1: To a mixture of (1S,3s)-adamantane-1-carboxylic acid (46 mg, 0.25 mmol) and HATU (108 mg, 0.28 mmol) in DMF (5 mL) was added DIEA (67 mg, 0.50 mmol) at an ambient temperature, and the reaction mixture was stirred for 0.5 h. Then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (90 mg, 0.25 mmol) was added into the solution and the reaction was stirred overnight. After completion, the mixture was quenched with H₂O (10 mL) and extracted with ethyl acetate (3*30 mL). The combined organic layers were washed with brine (3*20 mL), dried over anhydrous Na₂SO₄, and filtered. The filtrate was concentrated and purified by prep-TLC (DCM/MeOH=10:1) to give (1S,3S)—N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)adamantane-1-carboxamide (119 mg, 90% yield) as white solid. LC-MS (ESI) found: 511 [M+H]⁺.
Step 2: To a mixture of (1S,3S)—N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)adamantane-1-carboxamide (119 mg, 0.23 mmol) in THF (5 mL) was added 2 N HCl (0.5 mL) at an ambient temperature and stirred for 0.5 h. After completion, the mixture was concentrated and purified by prep-TLC (DCM/MeOH=10:1) to give compound A15 (42 mg, 38% yield) as white solid. LC-MS (ESI) found: 471 [M+H]⁺. ¹H NMR (400 MHz, CD₃OD): δ 8.10 (s, 1H), 8.00 (s, 1H), 7.52 (s, 1H), 4.32 (td, J=10.7, 5.2 Hz, 1H), 4.11 (dd, J=11.0, 5.1 Hz, 1H), 3.80 (d, J=3.0 Hz, 1H), 3.63 (dd, J=10.6, 3.1 Hz, 1H), 3.51 (dd, J=16.8, 7.9 Hz, 2H), 3.26 (d, J=8.9 Hz, 1H), 3.08 (t, J=10.8 Hz, 1H), 2.03 (d, J=3.0 Hz, 3H), 1.87 (s, 6H), 1.76 (q, J=12.3 Hz, 6H).

### Preparation of 6-(4-((4-(((2R,3R,4R,5S)-3,4-dihydroxy-5-((6-(trifluoromethyl)pyrazin-2-yl)amino)tetrahydro-2H-pyran-2-yl)methyl)piperazin-1-yl)methyl)piperidin-1-yl)pyridazine-3-carboxylic acid (A16)

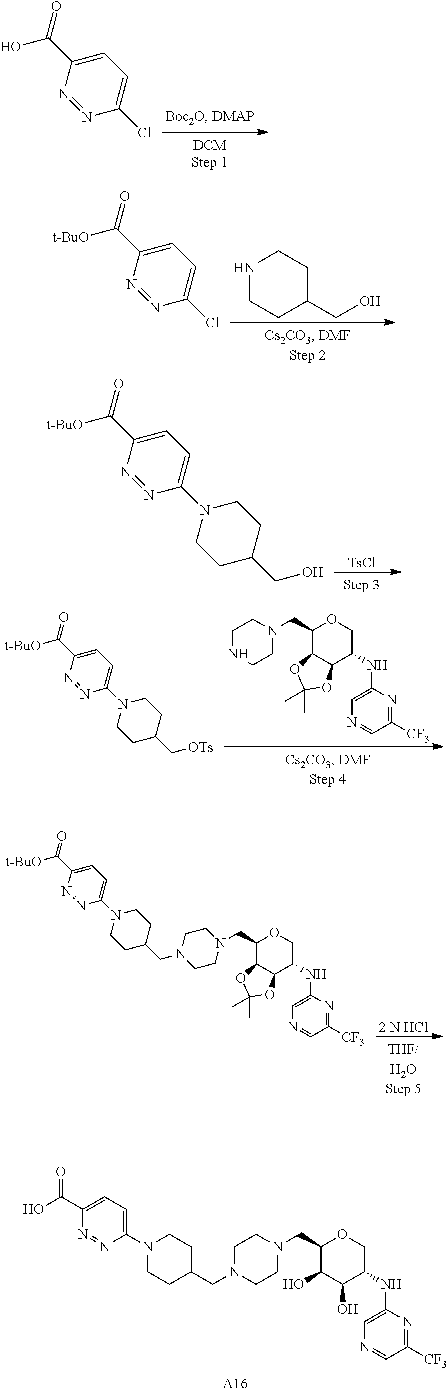

Step 1: A solution of 6-chloropyridazine-3-carboxylic acid (2 g, 12.6 mmol), Boc₂O (4.1 g, 18.9 mmol), DMAP (0.15 g, 1.3 mmol) and TEA (2.55 g, 25.2 mmol) in DCM (20 mL) was stirred at an ambient temperature overnight. The reaction was concentrated and purified by flash chromatography (PE/EA=10:1) to give tert-butyl 6-chloropyridazine-3-carboxylate (2.3 g, 84.94%) as white solid. LCMS: found no mass. ¹H NMR (400 MHz, DMSO-d₆): δ 8.19 (d, J=8.9 Hz, 1H), 8.05 (d, J=8.9 Hz, 1H), 1.57 (s, 9H).
Step 2: A suspension of tert-butyl 6-chloropyridazine-3-carboxylate (1 g, 4.7 mmol), piperidin-4-ylmethanol (650 mg, 5.6 mmol) and K₂CO₃ (0.65 g, 4.7 mmol) in DMF (5 mL) was stirred at 100° C. overnight. The reaction mixture was cooled and concentrated. The residue was purified by flash chromatography (PE//EA=2:1) to give tert-butyl 6-(4-(hydroxymethyl)piperidin-1-yl)pyridazine-3-carboxylate (1 g, 73%) as a pink solid. LC-MS (ESI) found: 587 [M+H]⁺.
Step 3: To a solution of tert-butyl 6-(4-(hydroxymethyl)piperidin-1-yl)pyridazine-3-carboxylate (300 mg, 1.0 mmol) and TEA (208 mg, 2.1 mmol) in DCM (5 mL) was added TsCl (293 mg, 1.5 mmol). The resulting mixture was stirred at an ambient temperature for 3 h. Then the reaction mixture washed with brine, dried over Na₂SO₄, filtered and concentrated to give crude tert-butyl 6-(4-((tosyloxy)methyl)piperidin-1-yl)pyridazine-3-carboxylate (458 mg) as a brown oil.
Step 4: A suspension of N-((3aS,4R,7S,7aR)-2,2-dimethyl-4-(piperazin-1-ylmethyl)tetrahydro-4H-[1,3]dioxolo[4,5-c]pyran-7-yl)-6-(trifluoromethyl)pyrazin-2-amine (200 mg, 0.5 mmol), tert-butyl 6-(4-((tosyloxy)methyl)piperidin-1-yl)pyridazine-3-carboxylate (255 mg, 0.6 mmol) and K₂CO₃ (199 mg, 1.4 mmol) in DMF (3 mL) was stirred at 100° C. for 3 h. The reaction was cooled and concentrated. The residue was purified by flash chromatography (MeOH/DCM=1:10) to give tert-butyl 6-(4-((4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)piperazin-1-yl)methyl)piperidin-1-yl)pyridazine-3-carboxylate (120 mg, 36% yield) as yellow oil. LC-MS (ESI) found: 693[M+H]⁺.
Step 5: A solution of tert-butyl 6-(4-((4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)piperazin-1-yl)methyl)piperidin-1-yl)pyridazine-3-carboxylate (140 mg, 0.2 mmol) in diluted aqueous HCl (3 mL, 3N) was stirred at 50° C. overnight. The reaction mixture was concentrated. The residue was purified by flash chromatography (MeOH in H₂O, from 5% to 30%) to give compound A16 (50 mg, 40%) as a white solid. LC-MS (ESI) found: 597[M+H]⁺.

### Preparation of N-(((2R,3R,4R,5S)-3,4-dihydroxy-5-((6-(trifluoromethyl)pyrazin-2-yl)amino)tetrahydro-2H-pyran-2-yl)methyl)quinazoline-6-carboxamide (A17)

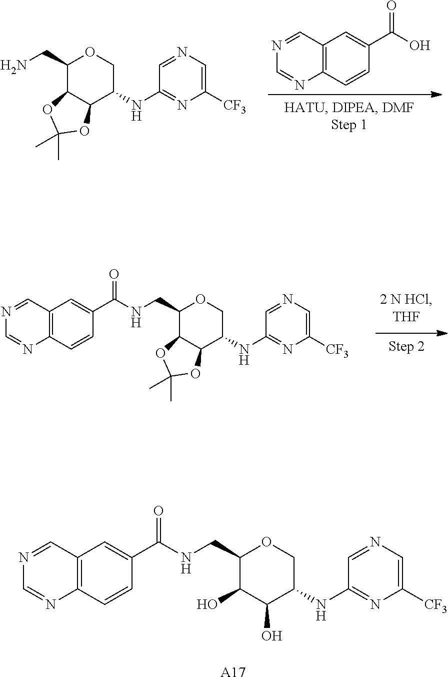 [SMILES: CC1(C)O[C@@H]2[C@H](O1)[C@@H](Nc1cncc(C(F)(F)F)n1)CO[C@@H]2CN ; O=C(O)c1ccc2ncncc2c1 ; CC1(C)O[C@@H]2[C@H](O1)[C@@H](Nc1cncc(C(F)(F)F)n1)CO[C@@H]2CNC(=O)c1ccc2ncncc2c1 ; O=C(NC[C@H]1OC[C@H](Nc2cncc(C(F)(F)F)n2)[C@@H](O)[C@H]1O)c1ccc2ncncc2c1 ; "A17"]

Step 1: To a mixture of quinazoline-6-carboxylic acid (30 mg, 0.18 mmol) and HATU (69 mg, 0.18 mmol) in DMF (2 mL) was added DIEA (70 mg, 0.54 mmol) at an ambient temperature, and the reaction mixture was stirred at an ambient temperature for 0.5 h, and then N-((3aS,4R,7S,7aR) 4-(aminomethyl)-2,2-dimethyltetrahydro-4H-[1,3]dioxolo[4,5-c]pyran-7-yl)-6-(trifluoromethyl)pyrazin-2-amine (60 mg, 0.18 mmol) was added into the solution. The reaction was stirred at an ambient temperature overnight. After completion, the mixture was quenched with H₂O (10 mL) and extracted with ethyl acetate (30 mL×3). The combined organic layer was washed with brine (20 mL×3), and dried over anhydrous Na₂SO₄, filtered. The filtrate was concentrated and purified by pre-HPLC (0.3% NH₃·H₂O) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinazoline-6-carboxamide (27 mg, yield 31%) as yellow oil. LC-MS (ESI) found: 505 [M+H]⁺.
Step 2: To a mixture of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinazoline-6-carboxamide (27 mg, 0.05 mmol) in THF (2 mL) was added 2N HCl (0.1 mL) at an ambient temperature. The reaction was stirred at an ambient temperature for 0.5 h. After completion, the mixture was concentrated and purified by reversed flash column (5% acetonitrile) to give compound A17 (16.9 mg, yield: 68%) as a white solid. LC-MS (ESI) found: 465 [M+H]⁺. ¹H NMR (400 MHz, CD₃OD) δ 8.69 (s, 1H), 8.20 (s, 1H), 8.06 (d, J=5.2 Hz, 3H), 7.48-7.39 (m, 1H), 6.26 (s, 1H), 4.38 (td, J=10.5, 5.0 Hz, 1H), 4.13 (dd, J=11.0, 5.0 Hz, 1H), 3.94 (d, J=2.7 Hz, 1H), 3.78-3.66 (m, 3H) 3.59 (dd, J=14.8, 9.2 Hz, 1H), 3.18 (t, J=10.8 Hz, 1H).

### Preparation of (1S,4R)—N-(((2R,3R,4R,5S)-3,4-dihydroxy-5-((6-(trifluoromethyl)pyrazin-2-yl)amino)tetrahydro-2H-pyran-2-yl)methyl)bicyclo[2.2.1]heptane-2-carboxamide (A18)

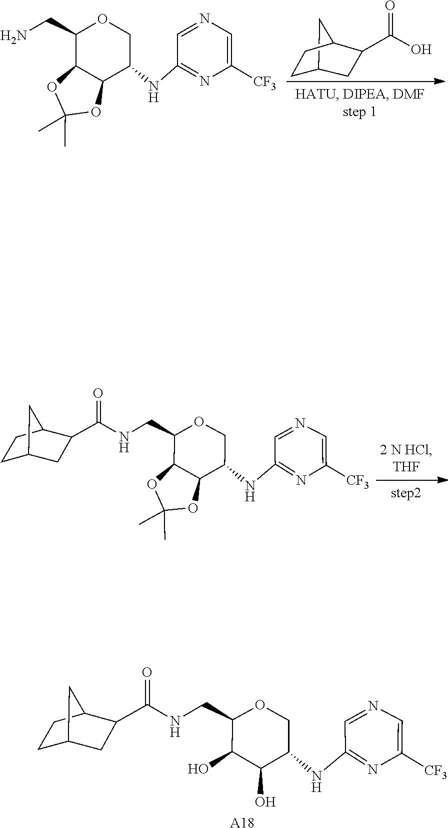

Step 1: To a solution of (1S,4R)-bicyclo[2.2.1]heptane-2-carboxylic acid (60 mg, 0.43 mmol) in DMF (5 mL) were added HATU (222 mg, 0.58 mmol) and DIEA (119 mg, 0.92 mmol), the mixture was stirred at 0° C. for 0.5 h. Then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (102 mg, 0.29 mmol) in DMF (0.5 ml) was added. The mixture was stirred at an ambient temperature for 18 h. The reaction mixture was diluted with ethyl acetate and washed with brine, dried over Na₂SO₄, filtered and concentrated. The residue was purified by prep-HPLC to give (1S,4R)—N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)bicyclo[2.2.1]heptane-2-carboxamide as white solid (60 mg, yield: 44%). LC-MS (ESI) found: 471 [M+H]⁺.
Step 2: To a solution of (1S,4R)—N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)bicyclo[2.2.1]heptane-2-carboxamide (60 mg, 0.12 mmol) in THF (2 ml) was added 2N HCl (6 ml). The mixture was stirred at an ambient temperature for 1 h. The reaction mixture was neutralized with NH₃·H₂O and concentrated. The residue was purified by prep-HPLC to give compound A18 as white solid (10 mg, yield: 19%). LC-MS (ESI) found: 431 [M+H]⁺. ¹H NMR (400 MHz, CD₃OD) δ 8.10 (s, 1H), 7.99 (s, 1H), 4.32 (td, J=6.4, 10.4 Hz, 1H), 4.12 (d, J=4.8, 10.8 Hz, 1H), 3.8-3.85 (m, 1H), 3.6-3.65 (m, 1H), 3.45-3.53 (m, 2H), 3.35-3.42 (m, 1H), 3.0-3.15 (m, 1H), 2.65-2.75 (m, 0.7H), 2.45-2.50 (m, 0.7H), 2.20-2.35 (m, 2H), 1.20-1.66 (m, 10H).

### Preparation of (2R,3R,4R,5S)-2-((pyrimidin-2-ylamino)methyl)-5-((6-(trifluoromethyl)pyrazin-2-yl)amino)tetrahydro-2H-pyran-3,4-diol (A19)

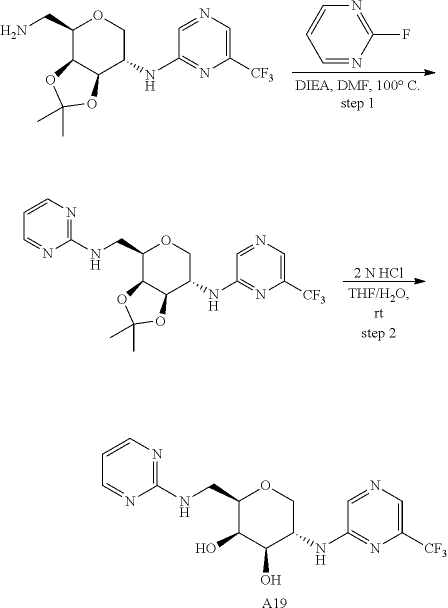

Step 1: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) in DMF (3 mL) were added 2-fluoropyrimidine (21 mg, 0.21 mmol) and DIEA (36 mg, 0.28 mmol) at an ambient temperature. The mixture was stirred at 100° C. overnight. The mixture was concentrated and the residue was purified by prep-TLC to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrimidin-2-amine (40 mg, 65%) as yellow oil. LC-MS (ESI) found: 427 [M+H]⁺.
Step 2: A solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrimidin-2-amine (40 mg, 0.09 mmol) in THF (1 mL) was added HCl (1 mL, 2 N). The mixture was stirred at an ambient temperature overnight. The mixture was concentrated in vacuo. The crude product was neutralized with NH₃·H₂O and purified by prep-HPLC to give compound A19 (15 mg, 41%) as white solid. LC-MS (ESI) found: 387 [M+H]⁺. ¹H NMR (400 MHz, CD₃OD): δ 8.28 (d, J=4.8 Hz, 2H), 8.10 (s, 1H), 8.00 (s, 1H), 6.63 (t, J=4.9 Hz, 1H), 4.35 (td, J=10.6, 5.1 Hz, 1H), 4.13 (dd, J=11.0, 5.1 Hz, 1H), 3.89 (d, J=2.9 Hz, 1H), 3.70-3.58 (m, 3H), 3.52 (dd, J=13.0, 6.7 Hz, 1H), 3.11 (t, J=10.8 Hz, 1H).

### Preparation of: N-(((2R,3R,4R,5S)-3,4-dihydroxy-5-((6-(trifluoromethyl)pyrazin-2-yl)amino)tetrahydro-2H-pyran-2-yl)methyl)pyrazine-2-carboxamide (A20)

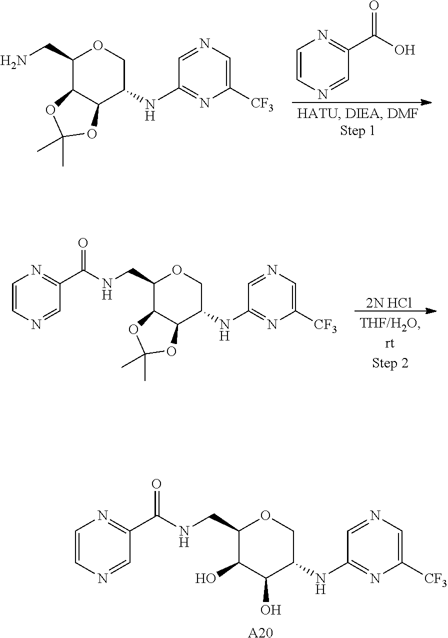

Step 1: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) in DMF (3 mL) were added pyrazine-2-carboxylic acid (26 mg, 0.21 mmol), HATU (64 mg, 0.17 mmol) and DIEA (36 mg, 0.28 mmol) at an ambient temperature. The mixture was stirred at an ambient temperature overnight. The reaction mixture was diluted with ethyl acetate and washed with brine, dried over Na₂SO₄, filtered and concentrated. The residue was purified by prep-TLC to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrazine-2-carboxamide (40 mg, 61%) as yellow oil. LC-MS (ESI) found: 455 [M+H]⁺.
Step 2: A solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrazine-2-carboxamide (40 mg, 0.088 mmol) in THF (1 mL) was added HCl (1 mL, 2 N). The mixture was stirred at an ambient temperature overnight. The mixture was concentrated in vacuo. The crude product was neutralized with NH₃·H₂O and purified by prep-HPLC to give compound A20 (20 mg, 55%) as white solid. LC-MS (ESI) found: 415 [M+H]⁺. ¹H NMR (400 MHz, CD₃OD): δ 9.24 (d, J=1.2 Hz, 1H), 8.78 (d, J=2.4 Hz, 1H), 8.70-8.66 (m, 1H), 8.11 (s, 1H), 8.00 (s, 1H), 4.36 (d, J=5.1 Hz, 1H), 4.14 (dd, J=11.0, 5.1 Hz, 1H), 3.92 (d, J=2.9 Hz, 1H), 3.77 (dd, J=12.6, 3.9 Hz, 1H), 3.69-3.59 (m, 3H), 3.11 (d, J=10.9 Hz, 1H).

### Preparation of 2-((2R,3R,4R,5S)-3,4-dihydroxy-5-((6-(trifluoromethyl)pyrazin-2-yl)amino)tetrahydro-2H-pyran-2-yl)acetonitrile (A21)

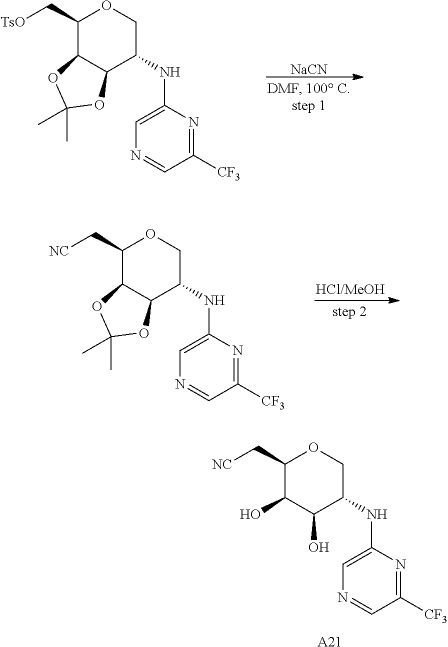 [SMILES: Cc1ccc(S(=O)(=O)OC[C@H]2OC[C@H](Nc3cncc(C(F)(F)F)n3)[C@H]3OC(C)(C)O[C@H]32)cc1 ; "NaCN" ; CC1(C)O[C@@H]2[C@H](O1)[C@@H](Nc1cncc(C(F)(F)F)n1)CO[C@@H]2CC#N ; "HCl/MeOH" ; N#CC[C@H]1OC[C@H](Nc2cncc(C(F)(F)F)n2)[C@@H](O)[C@H]1O ; "A21"]

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (1 g, 1.9 mmol) in DMF (15 mL) was added NaCN(0.49 g, 9.9 mmol) at an ambient temperature. The reaction was stirred at 100° C. for 4 days. The reaction was diluted with water and extracted with ethyl acetate. The organic layer was concentrated and purified by reverse column with MeOH/H₂O to give 2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetonitrile (400 mg, 1.1 mmol, 56% yield) as yellow oil. LC-MS (ESI) found: 358.9 [M+H]⁺.
Step 2: To a solution of 2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetonitrile(36 mg, 0.1 mmol) in Et₂O (3 mL) was added HCl (4 mL) and MeOH (4 mL) at an ambient temperature. The reaction was stirred at an ambient temperature for 18 h. Then the reaction was neutralized with NH₃·H₂O and concentrated. The residue was purified by prep-HPLC to give compound A21 (11.2 mg, 0.04 mmol, 35% yield) as white solid. LC-MS (ESI) found: 318.9 [M+H]⁺. ¹H NMR (400 MHz, CD₃OD): δ 8.11 (s, 1H), 8.01 (s, 1H), 4.32 (td, J=5.2, 10.4 Hz, 1H), 4.13 (dd, J=5.2, 11.2 Hz, 1H), 3.84 (d, J=2.4 Hz, 1H), 3.78 (dd, J=6.4, 8.0 Hz, 1H), 3.69 (dd, J=2.8, 10.8 Hz, 1H), 3.17 (t, J=11.2 Hz, 1H), 2.79 (td, J=8.4, 17.2 Hz, 2H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1H-imidazole-5-carboxamide (A22)

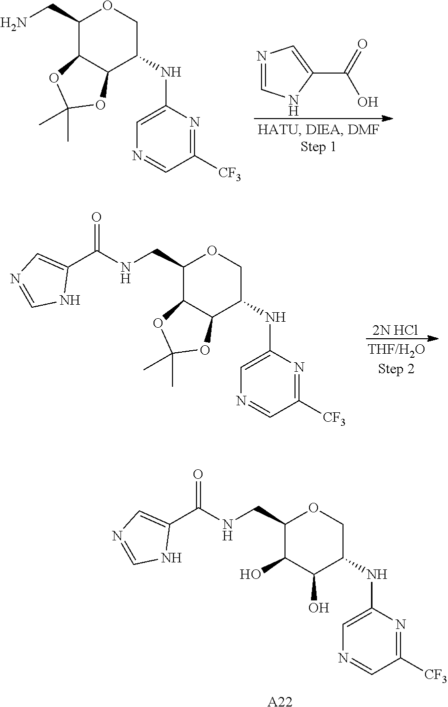 [SMILES: CC1(C)O[C@@H]2[C@H](O1)[C@@H](Nc1cncc(C(F)(F)F)n1)CO[C@@H]2CN ; O=C(O)c1cnc[nH]1 ; CC1(C)O[C@@H]2[C@H](O1)[C@@H](Nc1cncc(C(F)(F)F)n1)CO[C@@H]2CNC(=O)c1cnc[nH]1 ; "2N HCl" ; O=C(NC[C@H]1OC[C@H](Nc2cncc(C(F)(F)F)n2)[C@@H](O)[C@H]1O)c1cnc[nH]1 ; "A22"]

Step 1: To a solution of 1H-imidazole-5-carboxylic acid (33 mg, 0.3 mmol) in DMF (5 mL) was added HATU (167.8 mg, 0.5 mmol) and DIEA (114 mg, 0.9 mmol) at an ambient temperature. Then stirred for 20 min. Then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (102.5 mg, 0.3 mmol was added. Then the reaction solution was stirred for 18 h. Then the reaction solution was concentrated and the residue was purified by reverse phase column with MeOH/H₂O to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-imidazole-5-carboxamide (60 mg, 0.15 mmol, 46% yield) as white solid. LC-MS (ESI) found: 443.0 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-imidazole-5-carboxamide (60 mg, 0.14 mmol) in THF (4 mL) was added HCl (2M in water) (4 mL, 8.0 mmol) at an ambient temperature. The reaction was stirred rt for 2 h. The reaction was concentrated and the residue was purified by prep-HPLC to give compound A22 (37.7 mg, 0.1 mmol, 69% yield) as white solid. LC-MS (ESI) found: 402.8 [M+H]⁺. ¹H NMR (400 MHz, CD₃OD): δ 8.11 (s, 1H), 8.00 (s, 1H) 7.71 (s, 1H), 7.67 (s, 1H), 4.35 (td, J=5.2, 10.4 Hz, 1H), 4.15 (dd, J=4.8, 11.2 Hz, 1H), 3.90 (d, J=2.8 Hz, 1H), 3.58-3.74 (m, 3H), 3.47-3.55 (m, 1H), 3.13 (t, J=10.8 Hz, 1H).

### Preparation of (1,15-bis((2R,3R,4R,5S)-3,4-dihydroxy-5-((6-(trifluoromethyl)pyrazin-2-yl)amino)tetrahydro-2H-pyran-2-yl)-3,13-dioxo-6,10-dioxa-2,14-diazapentadecan-8-yl)glycine (A23)

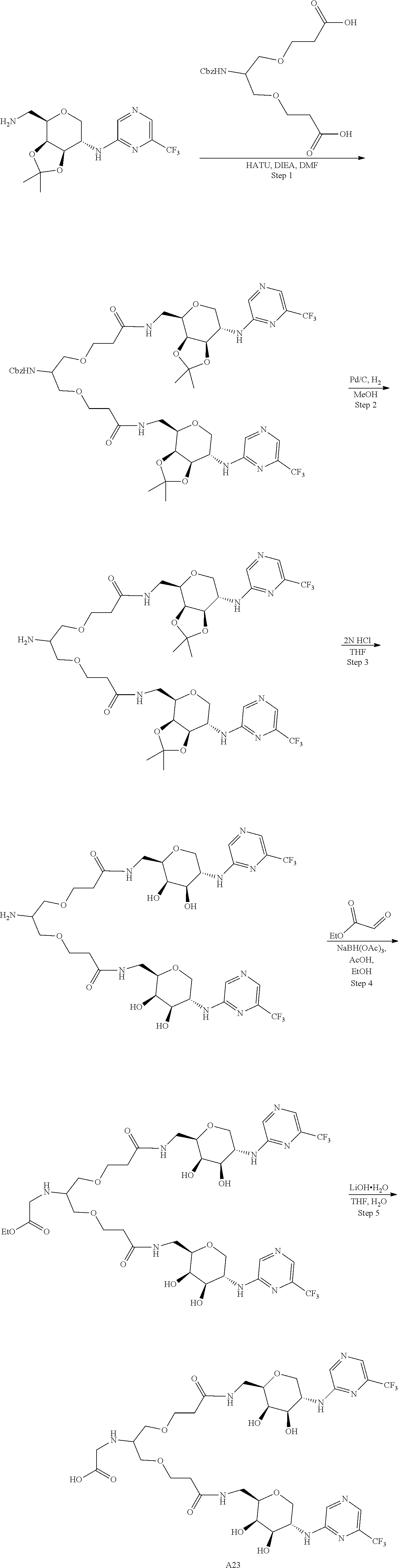

Step 1: A solution of 3,3′-((2-(((benzyloxy)carbonyl)amino)propane-1,3-diyl)bis(oxy))dipropionic acid (530 mg, 1.4 mmol), DIPEA (0.95 mL, 5.7 mmol) and HATU (1.3 g, 3.4 mmol) in DMF (15 mL) was stirred at an ambient temperature for 30 min. N-((3aS,4R,7S,7aR)-4-(aminomethyl)-2,2-dimethyltetrahydro-4H-[1,3]dioxolo[4,5-c]pyran-7-yl) 6-(trifluoromethyl)pyrazin-2-amine (1.2 g, 3.4 mmol) was then added at an ambient temperature, and the reaction was stirred overnight. Then the resulting mixture was concentrated in vacuo. The reside was purified by prep-HPLC to give benzyl (1,15-bis((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)-3,13-dioxo-6,10-dioxa-2,14-diazapentadecan-8-yl)carbamate (1.2 g, 1.2 mmol, 81.2% yield) as a brown oil. LC-MS (ESI) found: 1030 [M+H]⁺.
Step 2: A solution of benzyl (1,15-bis((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)-3,13-dioxo-6,10-dioxa-2,14-diazapentadecan-8-yl)carbamate (1.2 g, 1.2 mmol) in MeOH (15 mL) was added Pd/C (0.06 g, 0.6 mmol) under H2 balloon. The reaction was charged with H2 and stirred at an ambient temperature for 2 hr. The resulting mixture was filtered and concentrated in vacuo. The crude product 3,3′-((2-aminopropane-1,3-diyl)bis(oxy))bis(N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)propanamide) (1 g, 1.1 mmol, 95.8% yield) was used for next step with no further purification and work-up. LC-MS (ESI) found: 896 [M+H]⁺.
Step 3: To a solution of 3,3′-((2-aminopropane-1,3-diyl)bis(oxy))bis(N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)propanamide) (1 g, 1.1 mmol) in THF (10 mL) was added HCl (3 mL, 6 mmol). The reaction was stirred at an ambient temperature for 2 hr. The resulting mixture was filtered and concentrated in vacuo. The crude product was purified by prep-HPLC to give 3,3′-((2-aminopropane-1,3-diyl)bis(oxy))bis(N-(((2R,3R,4R,5S)-3,4-dihydroxy-5-((6-(trifluoromethyl)pyrazin-2-yl)amino)tetrahydro-2H-pyran-2-yl)methyl)propanamide) (850 mg, 1.0 mmol, 93.4% yield) as a colorless oil. LC-MS (ESI) found: 816 [M+H]⁺.
Step 4: A solution of 3,3′-((2-aminopropane-1,3-diyl)bis(oxy))bis(N-(((2R,3R,4R,5S)-3,4-dihydroxy-5-((6-(trifluoromethyl)pyrazin-2-yl)amino)tetrahydro-2H-pyran-2-yl)methyl)propanamide) (850 mg, 1.0 mmol) and ethyl 2-oxoacetate (0.3 mL, 1.6 mmol) in EtOH (8 mL) was added AcOH (0.03 mL, 0.5 mmol) under N₂. The reaction was stirred at an ambient temperature for 3 hr. Sodium triacetoxyborohydride (549.5 mg, 2.6 mmol) was added at 0° C. The reaction was stirred overnight. The resulting mixture was filtered and concentrated in vacuo. The crude product was purified by prep-HPLC to give ethyl (1,15-bis((2R,3R,4R,5S)-3,4-dihydroxy-5-((6-(trifluoromethyl)pyrazin-2-yl)amino)tetrahydro-2H-pyran-2-yl)-3,13-dioxo-6,10-dioxa-2,14-diazapentadecan-8-yl)glycinate (270 mg, 0.3 mmol, 28.7% yield) as a white solid. LC-MS (ESI) found: 902 [M+H]⁺.
Step 5: A solution of ethyl (1,15-bis((2R,3R,4R,5S)-3,4-dihydroxy-5-((6-(trifluoromethyl)pyrazin-2-yl)amino)tetrahydro-2H-pyran-2-yl)-3,13-dioxo-6,10-dioxa-2,14-diazapentadecan-8-yl)glycinate (270 mg, 0.3 mmol) in THF (6 mL) and H₂O (2 mL) was added LiOH (25 mg, 0.6 mmol) at an ambient temperature, and the reaction was stirred for 2 hr. The H of the reaction was adjusted to acidic with 2 M HCl. The crude product was purified by prep-HPLC to give compound A23 (180.8 mg, 0.2 mmol, 69.1% yield) as a white solid. LC-MS (ESI) found: 874 [M+H]⁺. ¹H NMR (400 MHz, CD₃OD): δ 8.11 (s, 2H), 7.98 (s, 2H), 4.33 (td, J=10.5, 5.1 Hz, 2H), 4.11 (dd, J=11.0, 5.1 Hz, 2H), 3.86 (d, J=2.6 Hz, 2H), 3.79-3.65 (m, 11H), 3.61 (s, 2H), 3.57-3.51 (m, 4H), 3.38-3.33 (m, 2H), 3.12 (t, J=10.8 Hz, 2H), 2.50 (t, J=5.7 Hz, 4H). ¹⁹F NMR (377 MHz, CD₃OD): δ −70.28 (s).

### Preparation of N-(((2R,3R,4R,5S)-3,4-dihydroxy-5-((6-(trifluoromethyl)pyrazin-2-yl)amino)tetrahydro-2H-pyran-2-yl)methyl)-3-fluorobenzamide (A24)

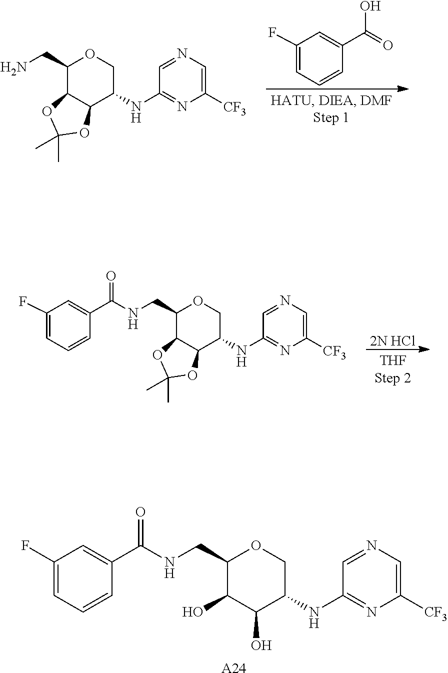 [SMILES: CC1(C)O[C@@H]2[C@H](O1)[C@@H](Nc1cncc(C(F)(F)F)n1)CO[C@@H]2CN ; O=C(O)c1cccc(F)c1 ; CC1(C)O[C@@H]2[C@H](O1)[C@@H](Nc1cncc(C(F)(F)F)n1)CO[C@@H]2CNC(=O)c1cccc(F)c1 ; "2N HCl" ; O=C(NC[C@H]1OC[C@H](Nc2cncc(C(F)(F)F)n2)[C@@H](O)[C@H]1O)c1cccc(F)c1 ; "A24"]

Step 1: To a solution of 3-fluorobenzoic acid (0.016 mL, 0.17 mmol) in DMF (2 mL) were added HATU (66 mg, 0.17 mmol) and DIEA (37 mg, 0.29 mmol) at an ambient temperature. After stirring 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, and then concentrated. The residue was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-3-fluorobenzamide (60 mg, 0.13 mmol, 88.9% yield) as pale yellow oil. LC-MS (ESI) found: 471 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-3-fluorobenzamide (60 mg, 0.13 mmol) in THF (3 mL) was added HCl (0.5 mL, 2 N) at an ambient temperature, and the mixture was stirred overnight, then concentrated. The residue was purified by prep-HPLC to give compound A24 (19.1 mg, 0.04 mmol, 34.8% yield) as white solid. LC-MS (ESI) found: 431 [M+H]+¹H NMR (400 MHz, CD₃OD): δ 8.11 (s, 1H), 8.00 (s, 1H), 7.66 (d, J=7.8 Hz, 1H), 7.58 (d, J=9.7 Hz, 1H), 7.49 (dt, J=13.7, 6.9 Hz, 1H), 7.29 (td, J=8.2, 1.9 Hz, 1H), 4.36 (td, J=10.5, 5.2 Hz, 1H), 4.14 (dd, J=11.0, 5.1 Hz, 1H), 3.91 (d, J=2.7 Hz, 1H), 3.74-3.65 (m, 3H) 3.52 (dd, J=13.2, 7.4 Hz, 1H), 3.12 (t, J=10.9 Hz, 1H). ¹⁹F NMR (377 MHz, CD₃OD): δ −70.32 (s), −114.50 (s).

### Preparation of N-(((2R,3R,4R,5S)-3,4-dihydroxy-5-((6-(trifluoromethyl)pyrazin-2-yl)amino)tetrahydro-2H-pyran-2-yl)methyl)pyrimidine-5-carboxamide (A25)

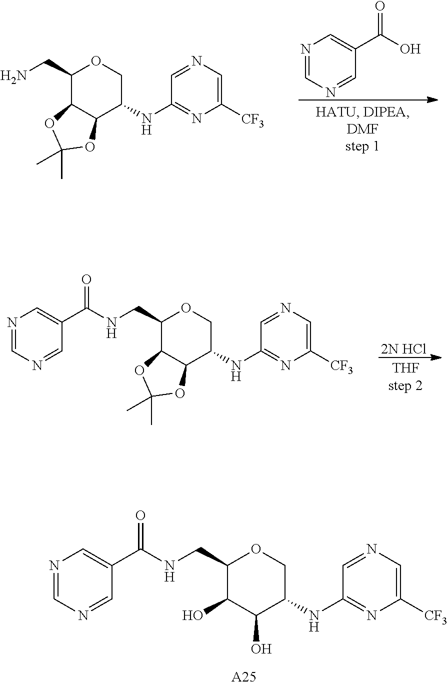

Step 1: To a solution of pyrimidine-5-carboxylic acid (21.38 mg, 0.17 mmol) in DMF (2 mL) were added HATU (66 mg, 0.17 mmol) and DIEA (37 mg, 0.29 mmol) at an ambient temperature. After stirring 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, then concentrated. The residue was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrimidine-5-carboxamide (61 mg, 0.13 mmol, 93.5% yield) as pale yellow oil. LC-MS (ESI) found: 455 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrimidine-5-carboxamide (61 mg, 0.13 mmol) in THF (3 mL) was added HCl (0.5 mL, 2 N) at an ambient temperature. The mixture was stirred overnight, then concentrated. The residue was purified by prep-HPLC to give compound A25 (8.9 mg, 0.02 mmol, 16.0% yield) as white solid. LC-MS (ESI) found: 415 [M+H]⁺. 1H NMR (400 MHz, CD₃OD): δ 9.27 (s, 1H), 9.17 (s, 2H), 8.11 (s, 1H), 8.00 (s, 1H) 4.36 (td, J=10.3, 5.0 Hz, 1H), 4.15 (dd, J=11.0, 5.2 Hz, 1H), 3.93 (d, J=2.9 Hz, 1H), 3.75 (dd, J=13.3, 4.3 Hz, 1H), 3.68 (dd, J=7.1, 3.8 Hz, 2H), 3.57 (dd, J=13.3, 7.8 Hz, 1H), 3.11 (d, J=10.8 Hz, 1H). ¹⁹F NMR (377 MHz, CD₃OD): δ −70.33 (s).

### Preparation of (((2R,3R,4R,5S)-3,4-dihydroxy-5-((6-(trifluoromethyl)pyrazin-2-yl)amino)tetrahydro-2H-pyran-2-yl)methyl)glycine (A26)

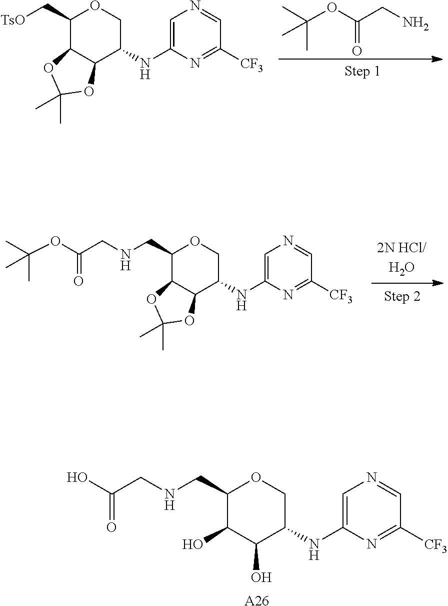

Step 1: A suspension of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (600 mg, 1.2 mmol), tert-butyl glycinate (154 mg, 1.2 mmol) and K₂CO₃ (486 mg, 3.6 mmol) in DMF (3 mL) was stirred at 100° C. overnight. The reaction mixture was cooled and concentrated. The residue was purified by flash chromatography (MeOH/DCM=1:10) to give tert-butyl (((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)glycinate (600 mg, crude) as a yellow oil. LC-MS (ESI) found: 463 [M+H]⁺.
Step 2: A solution of tert-butyl (((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)glycinate (600 mg, 1.3 mmol) in aqueous HCl (3 mL, 3N) was stirred at 50° C. for 30 min. The reaction mixture was cooled and concentrated. The residue was purified by flash chromatography reversed phase (C₁₈, MeOH in H₂O form 5% to 40%) to give compound A26 (197 mg, 41% yield) as a white solid. LC-MS (ESI) found: 367 [M+H]⁺. ¹H NMR (400 MHz, CD₃OD): δ 8.13 (s, 1H), 8.01 (s, 1H), 4.35 (td, J=10.5, 5.1 Hz, 1H), 4.20 (dd, J=11.0, 5.1 Hz, 1H), 3.97-3.92 (m, 1H), 3.80 (d, J=5.0 Hz, 1H), 3.72 (dd, J=10.5, 2.8 Hz, 1H), 3.57 (s, 2H), 3.41 (dd, J=12.9, 7.5 Hz, 1H), 3.27 (d, J=2.9 Hz, 1H) 3.19 (t, J=10.8 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)pyridazine-4-carboxamide (A27)

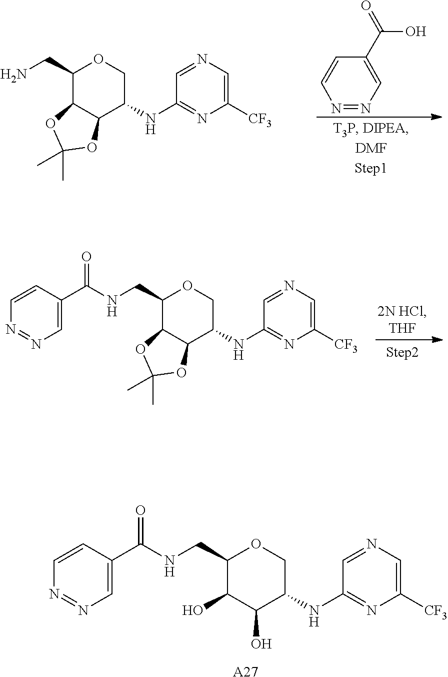

Step 1: To a solution of pyridazine-4-carboxylic acid (20 mg, 0.16 mmol) in DMF (3 mL) were added T3P (55 mg, 0.17 mmol) and DIPEA (37 mg, 0.29 mmol) at an ambient temperature. After stirring at an ambient temperature for 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, then concentrated. The residue was purified by flash to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazine-4-carboxamide (60 mg, 94%) as brown oil. LC-MS (ESI) found: 455 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazine-4-carboxamide (60 mg, 0.13 mmol) in THF (3 mL) was added HCl (0.5 mL, 2 N) at an ambient temperature. The mixture was stirred at an ambient temperature overnight, then neutralized with NH₃·H₂O and concentrated. The residue was purified by prep-HPLC to give compound A27 (23.2 mg, 42%) as white solid. LC-MS (ESI) found: 415 [M+H]⁺. ¹H NMR (400 MHz, CD₃OD): δ 9.53 (s, 1H) 9.37 (d, J=5.3 Hz, 1H), 8.11 (s, 1H), 8.04 (dd, J=5.2, 2.1 Hz, 1H), 8.00 (s, 1H), 4.35 (td, J=10.6, 5.1 Hz, 1H), 4.15 (dd, J=11.0, 5.1 Hz, 1H), 3.92 (d, J=2.8 Hz, 1H), 3.75 (dd, J=13.3, 4.0 Hz, 1H), 3.68 (dd, J=6.8, 4.0 Hz, 2H), 3.59 (dd, J=13.2, 7.8 Hz, 1H), 3.12 (t, J=10.9 Hz, 1H).

### Preparation of (2R,3R,4R,5S)-2-((pyridin-3-ylamino)methyl)-5-((6-(trifluoromethyl)pyrazin-2-yl)amino)tetrahydro-2H-pyran-3,4-diol (A28)

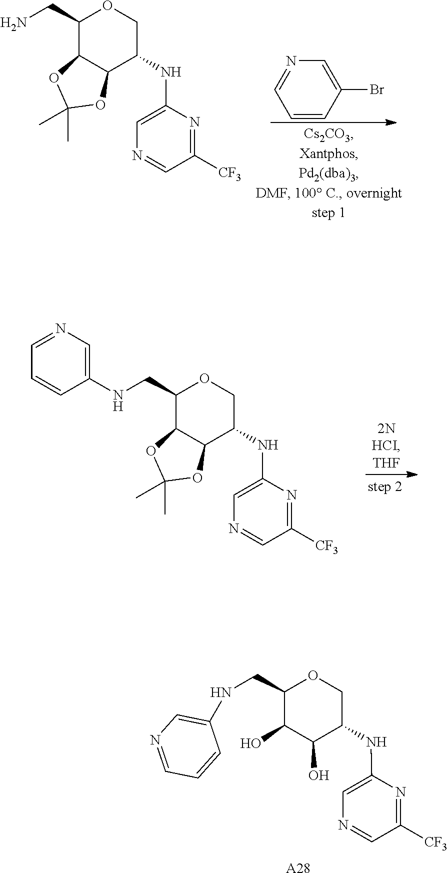 [SMILES: CC1(C)O[C@@H]2[C@H](O1)[C@@H](Nc1cncc(C(F)(F)F)n1)CO[C@@H]2CN ; Brc1cccnc1 ; CC1(C)O[C@@H]2[C@H](O1)[C@@H](Nc1cncc(C(F)(F)F)n1)CO[C@@H]2CNc1cccnc1 ; O[C@@H]1[C@H](O)[C@@H](Nc2cncc(C(F)(F)F)n2)CO[C@@H]1CNc1cccnc1 ; "A28"]

Step 1: A solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol), 3-bromopyridine (45 mg, 0.28 mmol), Pd₂(dba)₃ (13 mg, 0.014 mmol), Xantphos (16 mg, 0.029 mmol) and Cs₂CO₃ (94 mg, 0.28 mmol) in DMF (2 mL) was stirred at 100° C. overnight under N2 atmosphere. The mixture was concentrated and the residue was purified by prep-TLC to give N-((3aS,4R,7S,7aR)-2,2-dimethyl-4-((pyridin-3-ylamino)methyl)tetrahydro-4H-[1,3]dioxolo[4,5-c]pyran-7-yl)-6-(trifluoromethyl)pyrazin-2-amine (25 mg, 41%) as brown oil. LC-MS (ESI) found: 426 [M+H]⁺.
Step 2: To a solution of N-((3aS,4R,7S,7aR)-2,2-dimethyl-4-((pyridin-3-ylamino)methyl)tetrahydro-4H-[1,3]dioxolo[4,5-c]pyran-7-yl)-6-(trifluoromethyl)pyrazin-2-amine (25 mg, 0.06 mmol) in THF (3 mL) was added 2 N HCl (0.5 mL). The mixture was stirred at an ambient temperature for 3 h, then neutralized with NH₃·H₂O and concentrated. The residue was purified by flash (C18) chromatography to give compound A28 (3.5 mg, 13%) as white solid. LC-MS (ESI) found: 386 [M+H]⁺. ¹H NMR (400 MHz, CD₃OD): δ 8.11 (s, 1H), 8.00 (s, 1H), 7.98 (s, 1H), 7.78 (d, J=4.5 Hz, 1H), 7.21 (dd, J=14.4, 6.5 Hz, 2H), 4.36 (dd, J=10.6, 5.4 Hz, 1H), 4.15 (dd, J=11.0, 5.2 Hz, 1H), 3.93 (d, J=2.7 Hz, 1H), 3.68-3.59 (m, 2H), 3.41 (d, J=6.7 Hz, 2H), 3.12 (t, J=10.9 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-3-(trifluoromethyl)-1H-pyrazole-5-carboxamide (A29)

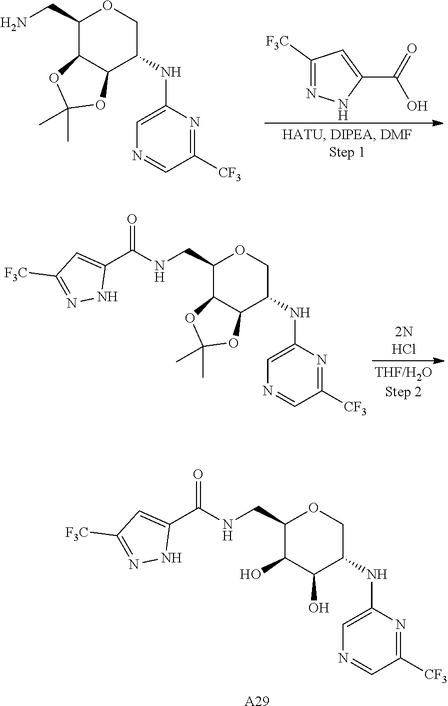 [SMILES: CC1(C)O[C@@H]2[C@H](O1)[C@@H](Nc1cncc(C(F)(F)F)n1)CO[C@@H]2CN ; O=C(O)c1cc(C(F)(F)F)n[nH]1 ; CC1(C)O[C@@H]2[C@H](O1)[C@@H](Nc1cncc(C(F)(F)F)n1)CO[C@@H]2CNC(=O)c1cc(C(F)(F)F)n[nH]1 ; O=C(NC[C@H]1OC[C@H](Nc2cncc(C(F)(F)F)n2)[C@@H](O)[C@H]1O)c1cc(C(F)(F)F)n[nH]1 ; "A29"]

Step 1: To a solution of 3-(trifluoromethyl)-1H-pyrazole-5-carboxylic acid (30 mg, 0.15 mmol) in DMF (5 mL) was added HATU (167.8 mg, 0.5 mmol) and DIEA (114 mg, 0.9 mmol) at an ambient temperature. The reaction was stirred for 20 min. Then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (51 mg, 0.15 mmol) was added. Then the reaction solution was stirred for 18 h. Then the reaction solution was neutralized with NH₃·H₂O and concentrated. The residue was purified by reverse phase column with MeOH/H₂O to give N-(((2R,3R,4R,5S)-3,4-dihydroxy-5-((6-(trifluoromethyl)pyrazin-2-yl)amino)tetrahydro-2H-pyran-2-yl)methyl)-3-(trifluoromethyl)-1H-pyrazole-5-carboxamide (25 mg, 0.05 mmol, 33% yield) as white solid. LC-MS (ESI) found: 511.0 [M+H]⁺.
Step 2: To a solution of N-(((2R,3R,4R,5S)-3,4-dihydroxy-5-((6-(trifluoromethyl)pyrazin-2-yl)amino)tetrahydro-2H-pyran-2-yl)methyl)-3-(trifluoromethyl)-1H-pyrazole-5-carboxamide (25 mg, 0.05 mmol) in THF (4 mL) was added HCl (2M in water) (4 mL, 8.0 mmol) at an ambient temperature. The reaction was stirred rt for 2 h. Then the reaction was neutralized with NH₃·H₂O and concentrated. The residue was purified by prep-HPLC to give compound A29 (12 mg, 0.03 mmol, 50% yield) as white solid. LC-MS (ESI) found: 471.2 [M+H]⁺. ¹H NMR (400 MHz, CD₃OD): δ 8.10 (s, 1H), 8.00 (s, 1H), 7.12 (s, 1H), 4.42-4.29 (m, 1H), 4.14 (dd, J=10.9, 5.1 Hz, 1H), 3.90 (d, J=2.7 Hz, 1H), 3.74-3.58 (m, 3H), 3.51 (dd, J=13.4, 7.5 Hz, 2H), 3.11 (t, J=10.9 Hz, 1H).

### Scheme-29: Preparation of 2-(2-((((2R,3R,4R,5S)-3,4-dihydroxy-5-((6-(trifluoromethyl)pyrazin-2-yl)amino)tetrahydro-2H-pyran-2-yl)methyl)amino)-2-oxoethoxy)acetic acid (A30)

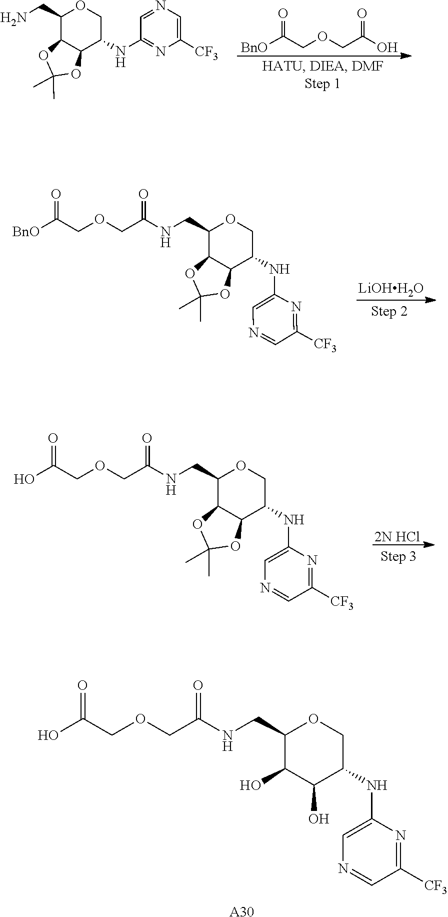

Step 1: A solution of 2-(2-(benzyloxy)-2-oxoethoxy)acetic acid (84.97 mg, 0.38 mmol), DIPEA (133.45 mg, 1.03 mmol) and HATU (144.14 mg, 0.38 mmol) in DMF (4 mL) was stirred at an ambient temperature for 30 min. N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (120 mg, 0.35 mmol) was added at an ambient temperature. The reaction was stirred overnight. The resulting mixture was concentrated in vacuo. The crude product was purified by flash chromatography (silica gel, 0˜10% MeOH in DCM) to give benzyl 2-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethoxy)acetate (120 mg, 62.82% yield) as a colorless oil. LC-MS (ESI) found: 555 [M+H]⁺.
Step 2: To a solution of benzyl 2-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethoxy)acetate (120 mg, 0.22 mmol) in THF (5 mL) was added LiOH·H₂O (46.2 mg, 1.10 mmol) and H₂O (0.1 mL) at an ambient temperature. The reaction was stirred at an ambient temperature overnight. The reaction was neutralized with diluted HCl and filtered. The filtrate was concentrated to give crude 2-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethoxy)acetic acid (90 mg, 89.55% yield) as a colorless oil. LC-MS (ESI) found: 465 [M+H]⁺.
Step 3: A solution of 2-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethoxy)acetic acid (90 mg, 0.19 mmol) in HCl/dioxane (5 mL, 4 N) was stirred at an ambient temperature for 2 h. The resulting mixture was neutralized with NH₃·H₂O and concentrate. The residue was purified by prep-HPLC (0.1% NH₃·H₂O) to give compound A30 (41.2 mg, 50.10% yield) as white solid. LC-MS (ESI) found: 425 [M+H]⁺. 1H NMR (400 MHz, CD₃OD) δ 8.10 (s, 1H), 8.00 (s, 1H), 4.32 (dd, J=10.6, 5.6 Hz, 1H), 4.19 (s, 2H), 4.15-4.11 (m, 1H), 4.09 (d, J=5.0 Hz, 2H), 3.86 (d, J=3.1 Hz, 1H), 3.64 (dd, J=10.6, 3.2 Hz, 1H), 3.60-3.52 (m, 2H), 3.46-3.40 (m, 1H), 3.10 (t, J=10.8 Hz, 1H).

### Preparation of 4-(4-((4-(((2R,3R,4R,5S)-3,4-dihydroxy-5-((6-(trifluoromethyl)pyrazin-2-yl)amino)tetrahydro-2H-pyran-2-yl)methyl)piperazin-1-yl)methyl)piperidin-1-yl)benzoic acid (A31)

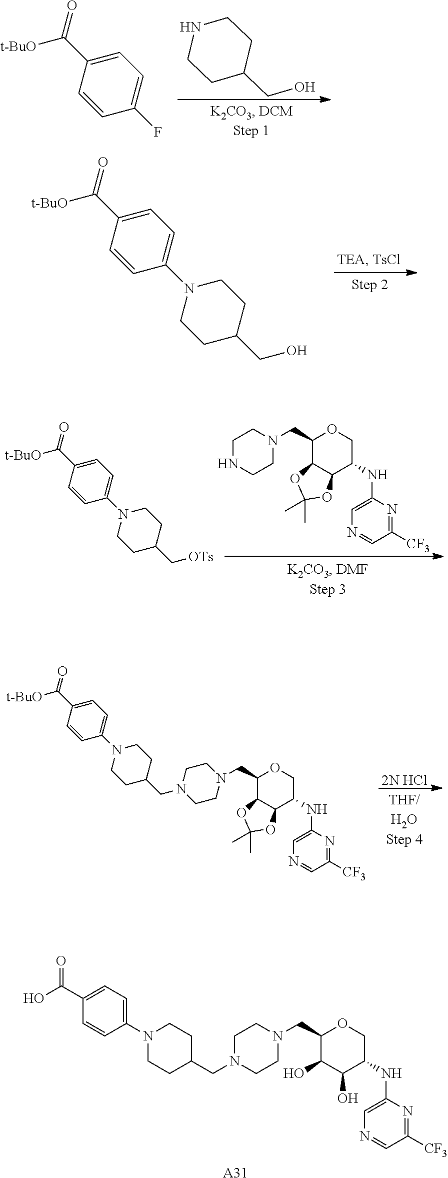

Step 1: A suspension of tert-butyl 4-fluorobenzoate (1 g, 4.7 mmol), piperidin-4-ylmethanol (0.65 mg, 5.6 mmol) and K₂CO₃ (0.65 g, 4.7 mmol) in DMF (5 mL) was stirred at 100° C. overnight. The reaction mixture was cooled and concentrated. The residue was purified by flash chromatography (EA/PE=1:2) to give tert-butyl 4-(4-(hydroxymethyl)piperidin-1-yl)benzoate (1 g, 70% yield) as pink solid. LC-MS (ESI) found: 291 [M+H]⁺.
Step 2: To a solution of tert-butyl 4-(4-(hydroxymethyl)piperidin-1-yl)benzoate (200 mg, 0.7 mmol) and TEA (0.19 mL, 1.4 mmol) in DCM (3 mL) was added TsCl (140 mg, 1.4 mmol). The reaction mixture was stirred at an ambient temperature for 2 h. Then the reaction was concentrated to give crude tert-butyl 4-(4-((tosyloxy)methyl)piperidin-1-yl)benzoate (306 mg) as brown oil.
Step 3: A suspension of N-((3aS,4R,7S,7aR)-2,2-dimethyl-4-(piperazin-1-ylmethyl)tetrahydro-4H-[1,3]dioxolo[4,5-c]pyran-7-yl)-6-(trifluoromethyl)pyrazin-2-amine (200 mg, 0.48 mmol), tert-butyl 4-(4-((tosyloxy)methyl)piperidin-1-yl)benzoate (255 mg, 0.6 mmol) and K₂CO₃ (198 mg, 1.4 mmol) in DMF (3 mL) was stirred at 100° C. for 3 h. The reaction was cooled and concentrated. The residue was purified by flash chromatography (DCM/MeOH=10:1) to give tert-butyl 4-(4-((4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)piperazin-1-yl)methyl)piperidin-1-yl)benzoate (120 mg, 36% yield) as yellow oil. LC-MS (ESI) found: 691 [M+H]⁺.
Step 4: A solution of tert-butyl 4-(4-((4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)piperazin-1-yl)methyl)piperidin-1-yl)benzoate (100 mg, 0.14 mmol) in diluted aqueous HCl (3 mL, 3M in water) was stirred at 50° C. for 1 h. The reaction solution was cooled, neutralized with NH₃·H₂O and concentrated to give crude mixture. The crude mixture was purified by prep-HPLC to give compound A31 (30 mg, 34% yield) as white solid. LC-MS (ESI) found: 595[M+H]⁺. ¹H NMR (400 MHz, CD₃OD): δ 8.11 (s, 1H), 8.00 (s, 1H), 7.82 (d, J=8.6 Hz, 2H), 6.52 (d, J=8.7 Hz, 2H), 4.32 (td, J=10.3, 5.0 Hz, 1H), 4.12 (dd, J=10.9, 5.1 Hz, 1H), 3.87 (d, J=2.7 Hz, 1H), 3.68-3.60 (m, 2H), 3.56-3.51 (m, 1H), 3.44 (t, J=7.6 Hz, 1H), 3.37-3.33 (m, 1H), 3.11 (t, J=10.8 Hz, 1H), 2.98 (t, J=8.7 Hz, 1H), 2.87-2.54 (m, 12H), 2.35-2.27 (m, 1H), 2.25-2.16 (m, 1H), 1.79-1.66 (m, 3H).

### Preparation of 4-((((2R,3R,4R,5S)-3,4-dihydroxy-5-((6-(trifluoromethyl)pyrazin-2-yl)amino)tetrahydro-2H-pyran-2-yl)methyl)carbamoyl)cyclohexane-1-carboxylic acid (A32

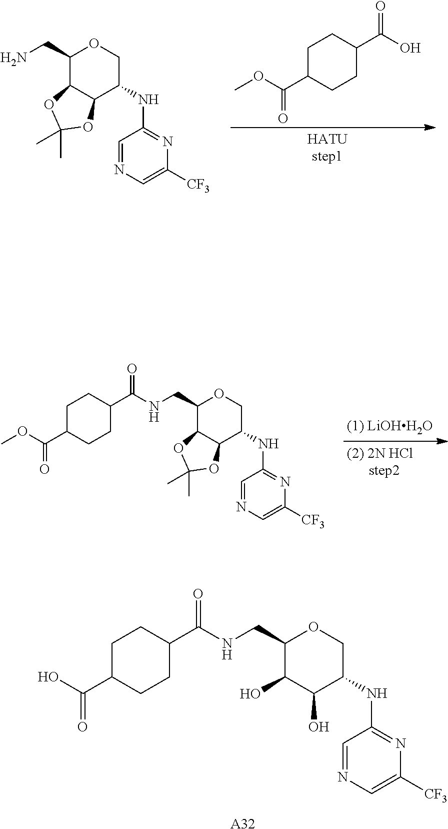 [SMILES: CC1(C)O[C@@H]2[C@H](O1)[C@@H](Nc1cncc(C(F)(F)F)n1)CO[C@@H]2CN ; COC(=O)C1CCC(C(=O)O)CC1 ; COC(=O)C1CCC(C(=O)NC[C@H]2OC[C@H](Nc3cncc(C(F)(F)F)n3)[C@H]3OC(C)(C)O[C@H]32)CC1 ; O=C(O)C1CCC(C(=O)NC[C@H]2OC[C@H](Nc3cncc(C(F)(F)F)n3)[C@@H](O)[C@H]2O)CC1 ; "A32"]

Step 1: To a solution o 4-methoxycarbonyl)cyclohexane-1-carboxylic acid (64 mg, 0.34 mmol) in DMF (5 mL) were added HATU (222 mg, 0.58 mmol) and DIEA (119 mg, 0.92 mmol), the mixture was stirred at 0° C. for 0.5 h. Then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (102 mg, 0.29 mmol) in DMF (0.5 ml) was added. The mixture was stirred at an ambient temperature for 1.5 h. The reaction mixture was purified by prep-HPLC to give methyl 4-((((3aS,4R,7S,7aR) 2,2-dimethyl-7-((6-(trifluoromethyl)pyrazin-2-yl)amino)tetrahydro-4H-[1,3]dioxolo[4,5-c]pyran-4-yl)methyl)carbamoyl)cyclohexane-1-carboxylate as white solid (90 mg, yield: 67%). LC-MS (ESI) found: 517 [M+H]⁺.
Step 2: To a solution of 4-((((2R,3R,4R,5S)-3,4-dihydroxy-5-((6-(trifluoromethyl)pyrazin-2-yl)amino)tetrahydro-2H-pyran-2-yl)methyl)carbamoyl)cyclohexane-1-carboxylic acid (90 mg, 0.17 mmol) in THF (2 ml) and H₂O (5 ml) was added LiOH·H₂O (36 mg, 0.87 mmol), and the mixture was stirred at an ambient temperature for 1 h. Then 2N HCL (6 ml) was added. The mixture was stirred at an ambient temperature for 1 h. The reaction mixture was concentrated and purified by prep-HPLC to give compound A32 as white solid (40 mg, yield: 57%). LC-MS (ESI) found: 431 [M+H]⁺. ¹H NMR (400 MHz, CD₃OD): δ 8.10 (s, 1H), 7.99 (s, 1H), 4.32 (td, J=6.4, 10.4 Hz, 1H), 4.12 (d, J=4.8, 10.8 Hz, 1H), 3.8-3.85 (m, 1H), 3.6-3.65 (m, 1H), 3.45-3.53 (m, 2H), 3.35-3.42 (m, 1H), 3.0-3.15 (m, 1H), 2.65-2.75 (m, 0.7H), 2.45-2.50 (m, 0.7H), 2.20-2.35 (m, 2H), 1.20-1.66 (m, 10H).

### Preparation of 4-((((2R,3R,4R,5S)-3,4-dihydroxy-5-((6-(trifluoromethyl)pyrazin-2-yl)amino)tetrahydro-2H-pyran-2-yl)methyl)carbamoyl)benzoic acid (A33)

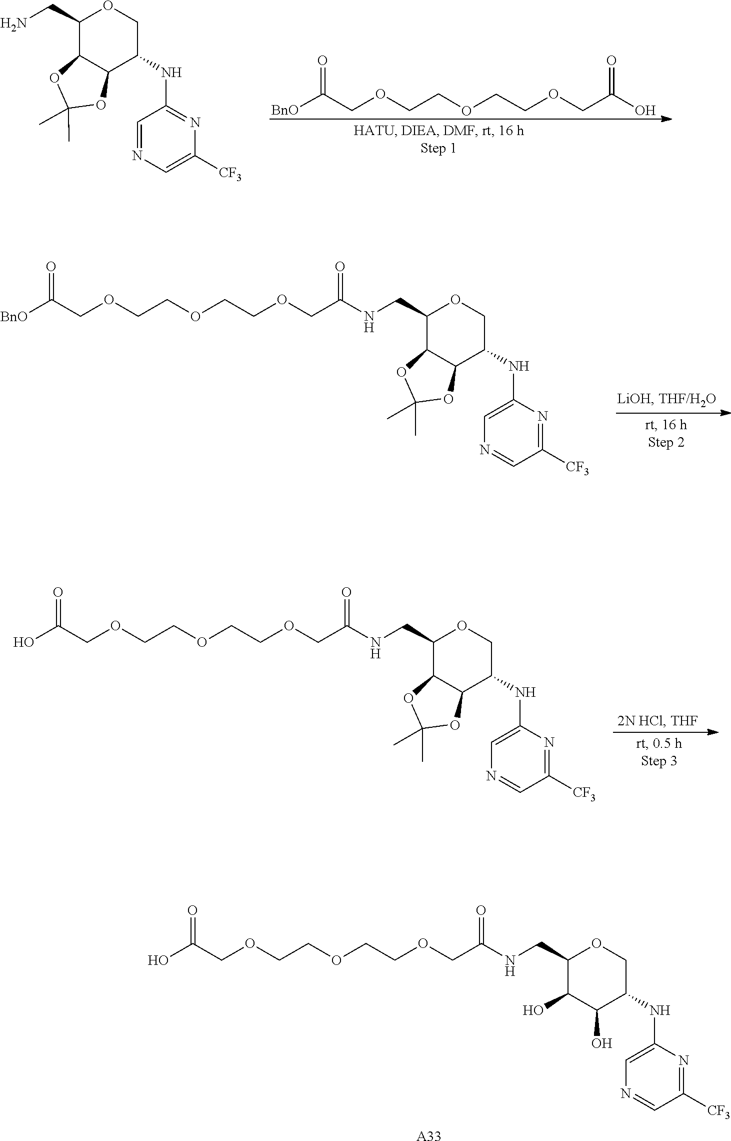 [SMILES: CC1(C)O[C@@H]2[C@H](O1)[C@@H](Nc1cncc(C(F)(F)F)n1)CO[C@@H]2CN ; O=C(O)COCCOCCOCC(=O)OCc1ccccc1 ; CC1(C)O[C@@H]2[C@H](O1)[C@@H](Nc1cncc(C(F)(F)F)n1)CO[C@@H]2CNC(=O)COCCOCCOCC(=O)OCc1ccccc1 ; CC1(C)O[C@@H]2[C@H](O1)[C@@H](Nc1cncc(C(F)(F)F)n1)CO[C@@H]2CNC(=O)COCCOCCOCC(=O)O ; O=C(O)COCCOCCOCC(=O)NC[C@H]1OC[C@H](Nc2cncc(C(F)(F)F)n2)[C@@H](O)[C@H]1O ; "A33"]

Step 1: To a mixture of 4-(methoxycarbonyl)benzoic acid (54 mg, 0.19 mmol) and HATU (144 mg, 0.38 mmol) in DMF (5 mL) was added DIEA (90 mg, 0.68 mmol) at an ambient temperature, and the reaction mixture was stirred at an ambient temperature for 0.5 h, then N-((3aS,4R,7S,7aR) 4-(aminomethyl)-2,2-dimethyltetrahydro-4H-[1,3]dioxolo[4,5-c]pyran-7-yl)-6-(trifluoromethyl)pyrazin-2-amine (60 mg, 0.19 mmol) was added into the solution and kept stirred overnight. After completion, the mixture was quenched with H₂O (10 mL) and extracted with EA (30 mL×3). The combined organic layers were washed with brine (20 mL×3), and dried over anhydrous Na₂SO₄, filtered. The filtrate was concentrated to give methyl benzyl 1-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)-3-oxo-5,8,11-trioxa-2-azatridecan-13-oate (160 mg) as crude solid, which was used for next step directly. LC-MS (ESI) found: 643 [M+H]⁺.
Step 2: To a mixture of methyl 1-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)-3-oxo-5,8,11-trioxa-2-azatridecan-13-oate (160 mg crude) in THF/H₂O (6 mL) was added LiOH (92 mg, 1.95 mmol) at an ambient temperature and the reaction mixture was stirred at an ambient temperature for 16 h. After completion, the mixture was neutralized with diluted HCl and concentrated under reduced pressure to give 1-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)-3-oxo-5,8,11-trioxa-2-azatridecan-13-oic acid (120 mg) as crude product, which was used for next step directly. LC-MS (ESI) found: 553 [M+H]⁺.
Step 3: To a mixture of 1-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)-3-oxo-5,8,11-trioxa-2-azatridecan-13-oic acid (120 mg, crude) in THF (5 mL) was added 2N HCl (0.5 mL) at an ambient temperature and the reaction was stirred for 0.5 h. After completion, the mixture was neutralized with NH₃·H₂O and concentrated and the residue was purified by reversed flash column (5% acetonitrile) to give compound A33 (21.8 mg, three steps yield: 22%) as white solid. LC-MS (ESI) found: 513 [M+H]⁺. ¹H NMR (400 MHz, CD₃OD): δ 8.11 (s, 1H), 8.00 (s, 1H), 4.32 (td, J=10.5, 5.1 Hz, 1H), 4.20-4.07 (m, 3H), 4.04 (d, J=16.5 Hz, 2H), 3.86 (d, J=2.7 Hz, 1H), 3.67 (dt, J=10.8, 7.4 Hz, 9H) 3.61-3.48 (m, 2H), 3.43 (td, J=11.7, 6.3 Hz, 1H), 3.11 (t, J=10.8 Hz, 1H).

### Preparation of 4-((((2R,3R,4R,5S)-3,4-dihydroxy-5-((6-(trifluoromethyl)pyrazin-2-yl)amino)tetrahydro-2H-pyran-2-yl)methyl)carbamoyl)benzoic acid(A34)

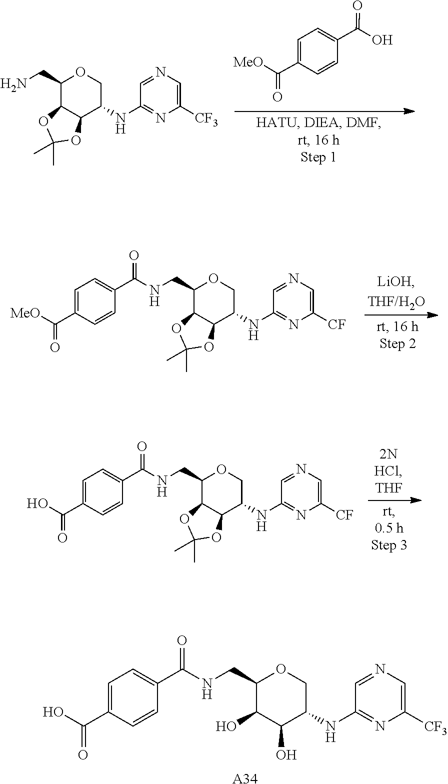

Step 1: To a mixture of 4-(methoxycarbonyl)benzoic acid (62 mg, 0.34 mmol) and HATU (144 mg, 0.38 mmol) in DMF (5 mL) was added DIEA (90 mg, 0.68 mmol) at an ambient temperature, and the reaction mixture was stirred at an ambient temperature for 0.5 h, then N-((3aS,4R,7S,7aR) 4-(aminomethyl)-2,2-dimethyltetrahydro-4H-[1,3]dioxolo[4,5-c]pyran-7-yl)-6-(trifluoromethyl)pyrazin-2-amine (120 mg, 0.34 mmol) was added into the solution and the reaction was kept stirring overnight. After completion, the mixture was quenched with H₂O (10 mL) and extracted with EA (30 mL×3). The combined organic layer was washed with brine (20 mL×3), and dried over anhydrous Na₂SO₄, filtered. The filtrate was concentrated to give methyl 4-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)benzoate (200 mg) as crude solid, which was used for next step directly. LC-MS (ESI) found: 511.2 [M+H]⁺.
Step 2: To a mixture of methyl 4-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)benzoate (200 mg crude) in THF/H₂O (6 mL) was added LiOH (92 mg, 1.95 mmol) at an ambient temperature, and the reaction mixture was stirred at an ambient temperature for 16 h. After completion, the mixture neutralized with diluted HCl and concentrated under reduced pressure to give 4-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)benzoic acid as crude product, which was used for next step directly. LC-MS (ESI) found: 497.2 [M+H]⁺.
Step 3: To a mixture of 4-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)benzoic acid (crude solid) in THF (5 mL) was added 2N HCl (0.5 mL) at an ambient temperature and stirred for 0.5 h. After completion, the mixture was neutralized with NH₃·H₂O and concentrated. The residue was purified by reversed flash column (5% acetonitrile) to give compound A34 (54 mg, three steps yield: 34.4%) as white solid. LC-MS (ESI) found: 457.2 [M+H]⁺. 1H NMR (400 MHz, CD₃OD): δ 8.10 (s, 1H), 8.01 (dd, J=10.4, 5.8 Hz, 3H), 7.84 (d, J=8.2 Hz, 2H), 4.36 (s, 1H), 4.14 (d, J=6.2 Hz, 1H), 3.91 (s, 1H), 3.69 (dd, J=20.5, 10.2 Hz, 3H), 3.54 (d, J=6.9 Hz, 1H), 3.14 (d, J=10.8 Hz, 1H).

### Preparation of (1,35-bis(((2R,3R,4R,5R,6R)-4,5-dihydroxy-6-(hydroxymethyl)-3-((6-(trifluoromethyl)pyrazin-2-yl)amino)tetrahydro-2H-pyran-2-yl)oxy)-13,23-dioxo-3,6,9,16,20,27,30,33-octaoxa-12,24-diazapentatriacontan-18-yl)glycine (A35)

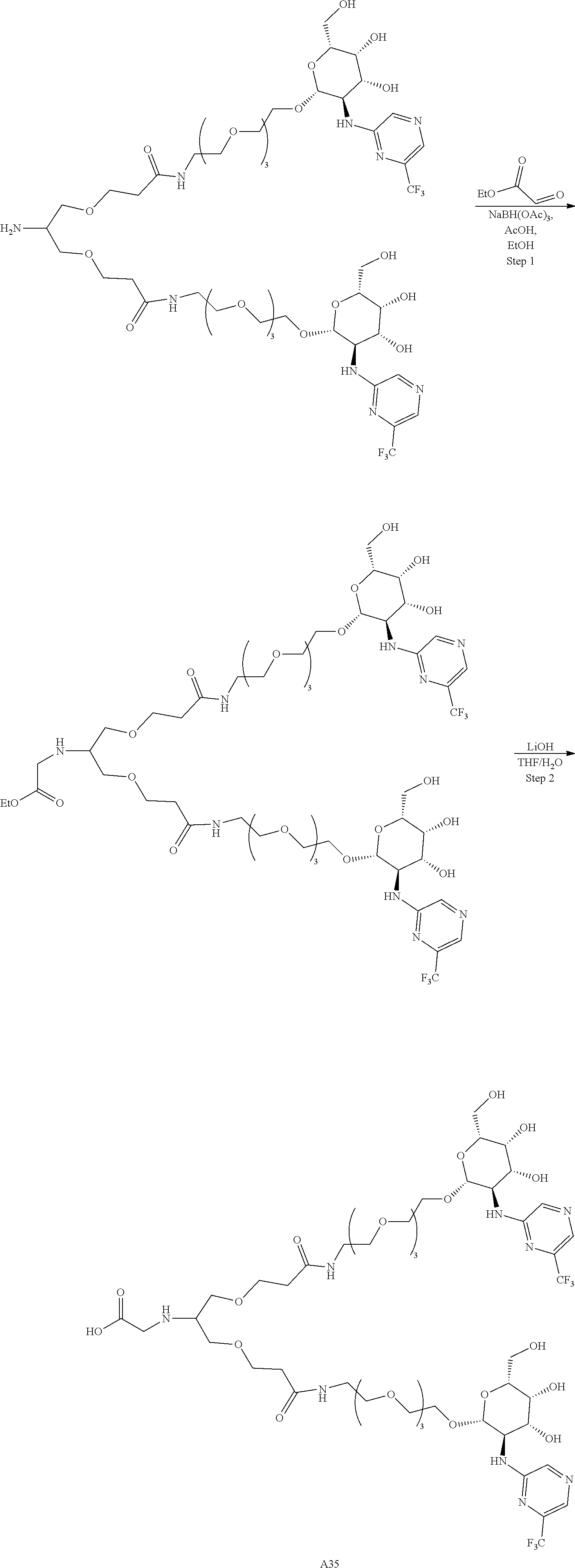

Step 1: A solution of 3,3′-((2-aminopropane-1,3-diyl)bis(oxy))bis(N-(2-(2-(2-(2-(((2R,3R,4R,5R,6R)-4,5-dihydroxy-6-(hydroxymethyl)-3-((6-(trifluoromethyl)pyrazin-2-yl)amino)tetrahydro-2H-pyran-2-yl)oxy)ethoxy)ethoxy)ethoxy)ethyl)propanamide) (100 mg, 0.08 mmol) and ethyl 2-oxoacetate (0.025 mL, 0.13 mmol) in EtOH (3 mL) was added AcOH (2.5 mg, 0.04 mmol) at an ambient temperature under N₂. The reaction was stirred at an ambient temperature for 3 hr. Sodium triacetoxyborohydride (35.2 mg, 0.17 mmol) was added at 0° C. The reaction was stirred overnight. The resulting mixture was filtered and concentrated in vacuo. The crude product was purified by prep-HPLC to give ethyl (1,35-bis(((2R,3R,4R,5R,6R)-4,5-dihydroxy-6-(hydroxymethyl)-3-((6-(trifluoromethyl)pyrazin-2-yl)amino)tetrahydro-2H-pyran-2-yl)oxy)-13,23-dioxo-3,6,9,16,20,27,30,33-octaoxa-12,24-diazapentatriacontan-18-yl)glycinate (30 mg, 28% yield) as a colorless oil. LC-MS (ESI) found: 1287 [M+H]⁺.
Step 2: A solution of ethyl (1,35-bis(((2R,3R,4R,5R,6R)-4,5-dihydroxy-6-(hydroxymethyl)-3-((6-(trifluoromethyl)pyrazin-2-yl)amino)tetrahydro-2H-pyran-2-yl)oxy)-13,23-dioxo-3,6,9,16,20,27,30,33-octaoxa-12,24-diazapentatriacontan-18-yl)glycinate (30 mg, 0.02 mmol) in THF (3 mL) and H₂O (1 mL) was added LiOH (1.96 mg, 0.05 mmol) at an ambient temperature. The reaction was stirred at an ambient temperature for 1.5 h. The reaction was adjusted to acidic with 2M HCl. The crude product was purified by prep-HPLC to give compound A35(2.6 mg, 8.9% yield) as a white solid. LC-MS (ESI) found: 1259 [M+H]⁺. ¹H NMR (400 MHz, CD₃OD): δ 8.20 (s, 2H), 8.01 (s, 2H), 4.16 (t, J=9.1 Hz, 2H), 3.98-3.90 (m, 4H), 3.81-3.65 (m, 17H), 3.61-3.42 (m, 30H), 3.39-3.35 (m, 4H), 2.48 (s, 4H). ¹⁹F NMR (377 MHz, CD₃OD): δ −69.97 (s).

### Preparation of N-(((2R,3R,4R,5S)-3,4-dihydroxy-5-((6-(trifluoromethyl)pyrazin-2-yl)amino)tetrahydro-2H-pyran-2-yl)methyl)benzamide (A36)

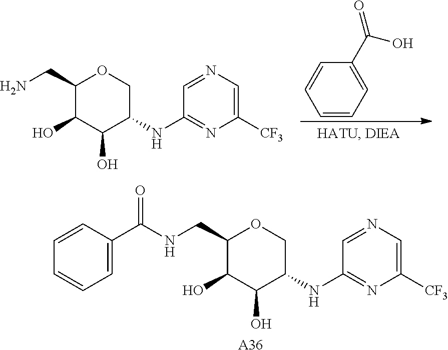

To a solution of benzoic acid (10 mg, 0.08 mmol) in DMF (2 mL) were added HATU (37 mg, 0.10 mmol) and DIPEA (17 mg, 0.13 mmol). After stirring at an ambient temperature for 20 min, (2R,3R,4R,5S)-2-(aminomethyl)-5-((6-(trifluoromethyl)pyrazin-2-yl)amino)tetrahydro-2H-pyran-3,4-diol (20 mg, 0.07 mmol). The mixture was stirred at an ambient temperature overnight, then concentrated. The mixture was purified by flash to give N-(((2R,3R,4R,5S)-3,4-dihydroxy-5-((6-(trifluoromethyl)pyrazin-2-yl)amino)tetrahydro-2H-pyran-2-yl)methyl)benzamide (2.3 mg, 9% yield) as white solid. LC-MS (ESI) found: 413 [M+H]⁺. ¹H NMR (400 MHz, CD₃OD): δ 8.10 (s, 1H), 8.00 (s, 1H), 7.83 (d, J=7.3 Hz, 2H), 7.57-7.41 (m, 3H), 4.35 (dd, J=13.0, 7.8 Hz, 1H), 4.14 (dd, J=11.0, 5.2 Hz, 1H), 3.91 (d, J=2.7 Hz, 1H) 3.77-3.61 (m, 3H), 3.51 (dd, J=13.3, 7.4 Hz, 1H), 3.12 (t, J=10.9 Hz, 1H).

### Preparation of (2R,3R,4R,5S)-2-((pyridazin-3-ylamino)methyl)-5-((6-(trifluoromethyl)pyrazin-2-yl)amino)tetrahydro-2H-pyran-3,4-diol (A37)

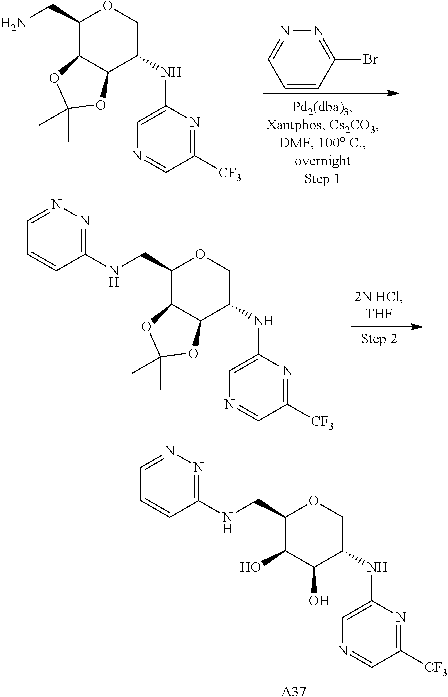 [SMILES: CC1(C)O[C@@H]2[C@H](O1)[C@@H](Nc1cncc(C(F)(F)F)n1)CO[C@@H]2CN ; Brc1cccnn1 ; CC1(C)O[C@@H]2[C@H](O1)[C@@H](Nc1cncc(C(F)(F)F)n1)CO[C@@H]2CNc1cccnn1 ; O[C@@H]1[C@H](O)[C@@H](Nc2cncc(C(F)(F)F)n2)CO[C@@H]1CNc1cccnn1 ; "A37"]

Step 1: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (30 mg, 0.08 mmol) in THF (2 mL) were added 3-bromopyridazine (21 mg, 0.13 mmol), Cs₂CO₃ (52 mg, 0.16 mmol), Xantphos (9 mg, 0.016 mmol) and Pd₂(dba)₃ (7 mg, 0.01 mmol) at an ambient temperature. The mixture was stirred at 100° C. under N₂ overnight. The mixture was purified by prep-TLC (100% EA) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazin-3-amine (20 mg, 55%) as yellow oil. LC-MS (ESI) found: 427 [M+H]⁺.
Step 2: A solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazin-3-amine (20 mg, 0.047 mmol) in THF (1 mL) was added HCl (1 mL, 2 N). The mixture was stirred at an ambient temperature overnight. The mixture was neutralized with NH₃·H₂O and concentrated in vacuo. The crude product was purified by prep-HPLC to give (2R,3R,4R,5S)-2-((pyridazin-3-ylamino)methyl)-5-((6-(trifluoromethyl)pyrazin-2-yl)amino)tetrahydro-2H-pyran-3,4-diol (1.3 mg, 7%) as white solid. LC-MS (ESI) found: 387 [M+H]⁺. ¹H NMR (400 MHz, CD₃OD): δ 8.37 (s, 1H), 8.10 (s, 1H), 8.00 (s, 1H), 7.29 (dd, J=9.3, 4.4 Hz, 1H), 6.95 (d, J=9.4 Hz, 1H), 4.35 (s, 1H), 4.14 (dd, J=10.7, 5.2 Hz, 1H), 3.91 (s, 1H), 3.75 (d, J=13.6 Hz, 1H), 3.68 (d, J=5.7 Hz, 2H), 3.54 (dd, J=13.7, 7.4 Hz, 1H), 3.11 (t, J=10.8 Hz, 1H).

### Preparation of 4′-((((2R,3R,4R,5S)-3,4-dihydroxy-5-((6-(trifluoromethyl)pyrazin-2-yl)amino)tetrahydro-2H-pyran-2-yl)methyl)carbamoyl)-[1,1′-biphenyl]-4-carboxylic acid (A38)

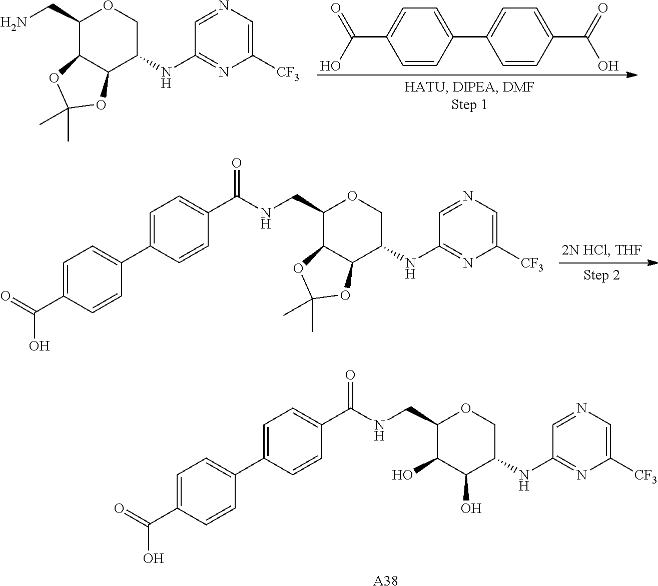 [SMILES: CC1(C)O[C@@H]2[C@H](O1)[C@@H](Nc1cncc(C(F)(F)F)n1)CO[C@@H]2CN ; O=C(O)c1ccc(-c2ccc(C(=O)O)cc2)cc1 ; CC1(C)O[C@@H]2[C@H](O1)[C@@H](Nc1cncc(C(F)(F)F)n1)CO[C@@H]2CNC(=O)c1ccc(-c2ccc(C(=O)O)cc2)cc1 ; O=C(O)c1ccc(-c2ccc(C(=O)NC[C@H]3OC[C@H](Nc4cncc(C(F)(F)F)n4)[C@@H](O)[C@H]3O)cc2)cc1 ; "A38"]

Step 1: A suspension of [1,1′-biphenyl]-4,4′-dicarboxylic acid (83 mg, 0.35 mmol) and HATU (52 mg, 0.14 mmol) in DMF (1 mL) was stirred at an ambient temperature for 2 h. DIEA (104 mg, 0.8 mmol) and N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (40 mg, 0.12 mmol) was added to above mixture. The reaction mixture was concentrated. The residue was purified by prep-TLC (MeOH in DCM=1:8) to give 4′-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-[1,1′-biphenyl]-4-carboxylic acid (30 mg, 46% yield) as a yellow solid. LC-MS (ESI) found: 573 [M+H]⁺.
Step 2: A solution of 4′-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-[1,1′-biphenyl]-4-carboxylic acid (30 mg, 0.052 mmol) in diluted aqueous HCl (2 mL, 2M in water) was stirred at 40° C. for 20 min. Reaction mixture was cooled and triturated with water (5 mL×3). The resulted solid was purified by prep-HPLC to give 4′-((((2R,3R,4R,5S)-3,4-dihydroxy-5-((6-(trifluoromethyl)pyrazin-2-yl)amino)tetrahydro-2H-pyran-2-yl)methyl)carbamoyl)-[1,1′-biphenyl]-4-carboxylic acid (7.5 mg, 27% yield) as a white solid. LC-MS (ESI) found: 533 [M+H]⁺. ¹H NMR (400 MHz, CD₃OD): δ 8.17-8.08 (m, 3H), 8.03-7.92 (m, 3H), 7.84-7.75 (m, 4H), 4.41-4.32 (m, 1H), 4.16 (dd, J=10.9, 4.8 Hz, 1H), 3.93 (s, 1H), 3.75 (d, J=13.5 Hz, 1H) 3.71-3.64 (m, 2H), 3.55 (dd, J=13.0, 7.4 Hz, 1H), 3.13 (t, J=10.6 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-4-fluoropicolinamide (A39)

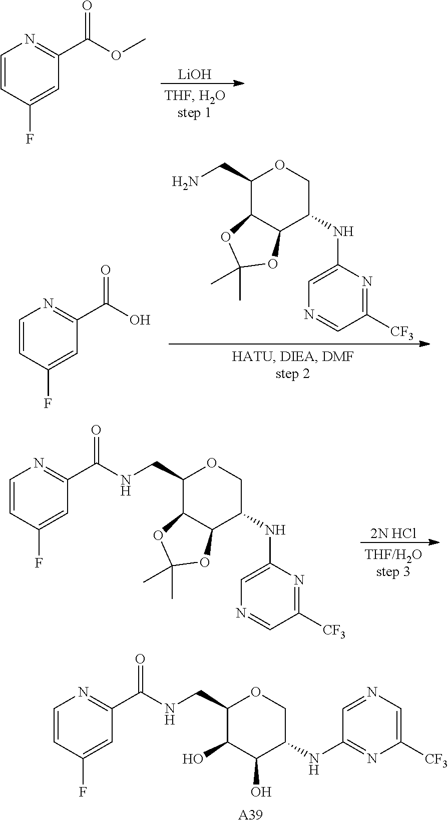

Step 1: To solution of methyl 4-fluoropicolinate (100 mg, 0.65 mmol) in THF (3 mL) and H₂O (1 mL) was added LiOH (54.1 mg, 1.29 mmol) at an ambient temperature. The reaction was stirred at an ambient temperature for 2 h. The reaction was adjusted to acidic with 2 M HCl. The resulting mixture was filtered and concentrated in vacuo. The crude product 4-fluoropyridine-2-carboxylic acid (85 mg, crude) was used for next step with no further purification. LC-MS (ESI) found: 142 [M+H]⁺.
Step 2: A solution of 4-fluoropicolinic acid (30.4 mg, 0.22 mmol), DIPEA (56 mg, 0.43 mmol) and HATU (71 mg, 0.19 mmol) in DMF (3 mL) was stirred at an ambient temperature for 30 min. N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added at an ambient temperature. The reaction was stirred at 60° C. overnight. The resulting mixture was concentrated in vacuo. The crude product was purified by prep-HPLC to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-4-fluoropicolinamide (5 mg, 7.4% yield) as a brown solid. LC-MS (ESI) found: 472 [M+H]⁺.
Step 3: A solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-4-fluoropicolinamide (5 mg, 0.01 mmol) in THF (2 mL) was added HCl (0.5 mL, 1 mmol). The reaction was stirred at an ambient temperature for 2 h. The resulting mixture was neutralized with NH₃·H₂O and concentrated in vacuo. The residue was purified by prep-HPLC to give N-(((2R,3R,4R,5S)-3,4-dihydroxy-5-((6-(trifluoromethyl)pyrazin-2-yl)amino)tetrahydro-2H-pyran-2-yl)methyl)-4-fluoropicolinamide (1.9 mg, 41.5% yield) as a white solid. LC-MS (ESI) found: 432 [M+H]⁺. ¹H NMR (400 MHz, CD₃OD): δ 8.65 (dd, J=7.9, 5.7 Hz, 1H), 8.11 (s, 1H), 8.00 (s, 1H), 7.85 (dd, J=9.4, 2.2 Hz, 1H) 7.41-7.33 (m, 1H), 4.36 (td, J=10.2, 4.8 Hz, 1H), 4.15 (dd, J=11.0, 5.1 Hz, 1H), 3.91 (d, J=2.7 Hz, 1H), 3.75 (dd, J=12.4, 3.7 Hz, 1H), 3.68-3.58 (m, 3H), 3.13 (t, J=10.9 Hz, 1H). ¹⁹F NMR (377 MHz, CD₃OD): δ −70.31 (s), −102.61 (s).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1H-imidazole-2-carboxamide (A40)

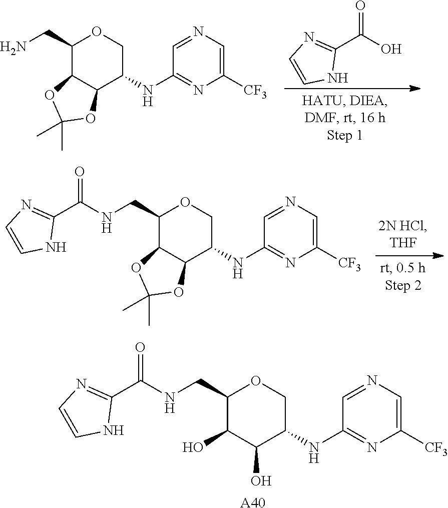

Step 1: To a mixture of 1H-imidazole-2-carboxylic acid (9.6 mg, 0.08 mmol) and HATU (36 mg, 0.09 mmol) in DMF (5 mL) was added DIEA (22.4 mg, 0.16 mmol) at an ambient temperature, and the reaction mixture was stirred at an ambient temperature for 0.5 h, then N-((3aS,4R,7S,7aR) 4-(aminomethyl)-2,2-dimethyltetrahydro-4H-[1,3]dioxolo[4,5-c]pyran-7-yl)-6-(trifluoromethyl)pyrazin-2-amine (30 mg, 0.08 mmol) was added into the solution and stirred overnight. After completion, the mixture was quenched with H₂O (10 mL) and extracted with EA (30 mL×3). The combined organic layers were washed with brine (20×3 mL), and dried over anhydrous Na₂SO₄, filtered. The filtrate was concentrated to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-imidazole-2-carboxamide (50 mg) as crude solid which used to next step directly. LC-MS (ESI) found: 443.2 [M+H]⁺.
Step 2: To a mixture of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-imidazole-2-carboxamide (50 mg crude) in THF (5 mL) was added 2N HCl (1 mL) at an ambient temperature, and the reaction mixture was stirred at an ambient temperature for 0.5 h. After completion, the mixture was neutralized with NH₃·H₂O and concentrated under reduced pressure. The residue was purified by reversed flash column (10% acetonitrile) to give N-(((2R,3R,4R,5S)-3,4-dihydroxy-5-((6-(trifluoromethyl)pyrazin-2-yl)amino)tetrahydro-2H-pyran-2-yl)methyl)-1H-imidazole-2-carboxamide (12 mg, 35.3% yield in two steps) as white solid. LC-MS (ESI) found: 403.2 [M+H]⁺. 1H NMR (400 MHz, MeOD) δ 8.10 (s, 1H), 8.00 (s, 1H), 7.18 (s, 2H), 4.40-4.30 (m, 1H), 4.14 (dd, J=11.0, 5.0 Hz, 1H), 3.90 (s, 1H), 3.73-3.54 (m, 4H), 3.13 (t, J=10.9 Hz, 1H).

### Preparation of (2R,3R,4R,5S)-2-((pyridazin-4-ylamino)methyl)-5-((6-(trifluoromethyl)pyrazin-2-yl)amino)tetrahydro-2H-pyran-3,4-diol (A41)

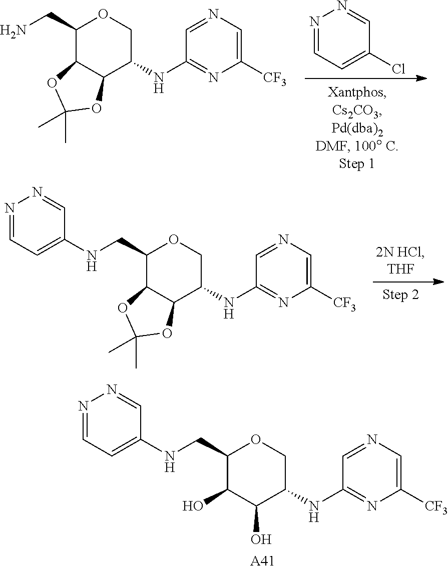

Step 1: To a mixture of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (60 mg, 0.18 mmol), Xantphos (10 mg, 0.018 mmol), CS₂CO₃ (175 mg, 0.54 mmol), 4-chloropyridazine(30 mg, 0.27 mmol) in DMF (3 mL) was added Pd(dba)₂ (10 mg, 0.018 mmol) at an ambient temperature and the reaction mixture was charged with N2 and stirred at 100° C. for 5 h. After completion, the mixture was concentrated in vacuo and the residue was purified by prep-HPLC to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazin-4-amine (28 mg, yield 36.5%). LC-MS (ESI) found: 427 [M+H]⁺.
Step 2: To a mixture of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazin-4-amine (28 mg, 0.06 mmol) in THF (2 mL) was added 2N HCl (0.5 mL) at an ambient temperature and stirred for 0.5 h. After completion, the mixture was neutralized with NH₃·H₂O and concentrated. The residue was purified by reversed flash column (5% acetonitrile) to give (2R,3R,4R,5S)-2-((pyridazin-4-ylamino)methyl)-5-((6-(trifluoromethyl)pyrazin-2-yl)amino)tetrahydro-2H-pyran-3,4-diol (4.5 mg, yield: 18%) as white solid. LC-MS (ESI) found: 387 [M+H]⁺. ¹H NMR (400 MHz, CD₃OD): δ 8.55 (d, J=2.7 Hz, 1H), 8.50 (d, J=6.4 Hz, 1H), 8.11 (s, 1H), 8.00 (s, 1H), 6.83 (dd, J=6.5, 3.1 Hz, 1H), 4.36 (td, J=10.6, 5.1 Hz, 1H), 4.14 (dd, J=11.1, 5.2 Hz, 1H), 3.91 (d, J=2.8 Hz, 1H), 3.65 (ddd, J=12.6, 9.4, 3.8 Hz, 2H), 3.54 (dd, J=13.9, 8.1 Hz, 1H), 3.49-3.38 (m, 1H) 3.12 (t, J=10.9 Hz, 1H).

### Preparation of 4-((((2R,3R,4R,5S)-3,4-dihydroxy-5-((6-(trifluoromethyl)pyrazin-2-yl)amino)tetrahydro-2H-pyran-2-yl)methyl)carbamoyl)benzoic acid (A42)

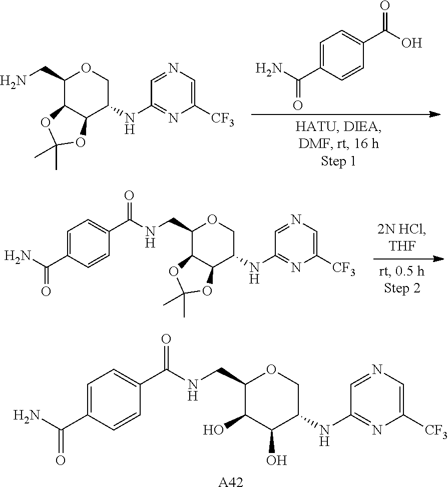

Step 1: To a mixture of 4-(methoxycarbonyl)benzoic acid (30 mg, 0.18 mmol) and HATU (69 mg, 0.18 mmol) in DMF (2 mL) was added DIEA (70 mg, 0.54 mmol) at an ambient temperature, and the reaction mixture was stirred at an ambient temperature for 0.5 h, and then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (60 mg, 0.18 mmol) was added into the solution and stirred overnight. After completion, the mixture was quenched with H₂O (10 mL) and extracted with EA (30 mL×3). The combined organic layers were washed with brine (20 mL×2), and dried over anhydrous Na₂SO₄, filtered. The filtrate was concentrated to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)terephthalamide (120 mg) as crude solid which used to next step directly. LC-MS (ESI) found: 496 [M+H]⁺.
Step 2: To a mixture of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)terephthalamide (crude solid) in THF (5 mL) was added 2N HCl (0.5 mL) at an ambient temperature and stirred for 0.5 h. After completion, the mixture was neutralized with NH₃·H₂O and concentrated. The residue was purified by reversed flash column (5% acetonitrile) to give N-(((2R,3R,4R,5S)-3,4-dihydroxy-5-((6-(trifluoromethyl)pyrazin-2-yl)amino)tetrahydro-2H-pyran-2-yl)methyl)terephthalamide (14.8 mg, two steps yield: 18%) as white solid. LC-MS (ESI) found: 456 [M+H]⁺. ¹H NMR (400 MHz, CD₃OD): δ 8.34 (s, 1H), 8.10 (s, 1H), 8.07-7.90 (m, 3H), 7.57 (t, J=7.8 Hz, 1H), 4.36 (td, J=10.7, 5.4 Hz, 1H), 4.15 (dd, J=11.2, 5.1 Hz, 1H), 3.92 (d, J=2.8 Hz, 1H), 3.81-3.62 (m, 3H) 3.60-3.44 (m, 2H), 3.12 (t, J=10.8 Hz, 1H).

### Preparation of 3-cyano-N-(((2R,3R,4R,5S)-3,4-dihydroxy-5-((6-(trifluoromethyl)pyrazin-2-yl)amino)tetrahydro-2H-pyran-2-yl)methyl)-1H-pyrazole-5-carboxamide (A43)

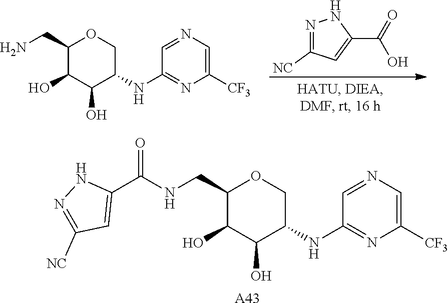

To a mixture of 3-cyano-1H-pyrazole-5-carboxylic acid (15 mg, 0.11 mmol) and HATU (41 mg, 0.11 mmol) in DMF (5 mL) was added DIEA (25 mg, 0.23 mmol) at an ambient temperature, and the reaction mixture was stirred at an ambient temperature for 0.5 h, then (2R,3R,4R,5S)-2-(aminomethyl)-5-((6-(trifluoromethyl)pyrazin-2-yl)amino)tetrahydro-2H-pyran-3,4-diol (30 mg, 0.10 mmol) was added into the solution and stirred overnight. After completion, the mixture was quenched with H₂O (10 mL) and extracted with EA (30 mL×3). The combined organic layers were washed with brine (20 mL×2), and dried over anhydrous Na₂SO₄, filtered. The filtrate was concentrated and purified by reversed phase flash column (5% acetonitrile) to give 3-cyano-N-(((2R,3R,4R,5S)-3,4-dihydroxy-5-((6-(trifluoromethyl)pyrazin-2-yl)amino)tetrahydro-2H-pyran-2-yl)methyl)-1H-pyrazole-5-carboxamide (11 mg, 26.8% yield) as white solid. LC-MS (ESI) found: 428.2 [M+H]⁺. ¹H NMR (400 MHz, CD₃OD): δ 8.10 (s, 1H), 8.00 (s, 1H), 7.25 (s, 1H), 4.34 (td, J=10.1, 4.7 Hz, 1H), 4.14 (dd, J=11.0, 5.2 Hz, 1H), 3.89 (d, J=2.6 Hz, 1H), 3.73-3.59 (m, 3H) 3.51 (dd, J=13.3, 7.8 Hz, 1H), 3.11 (t, J=10.7 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1H-indazole-3-carboxamide (A44)

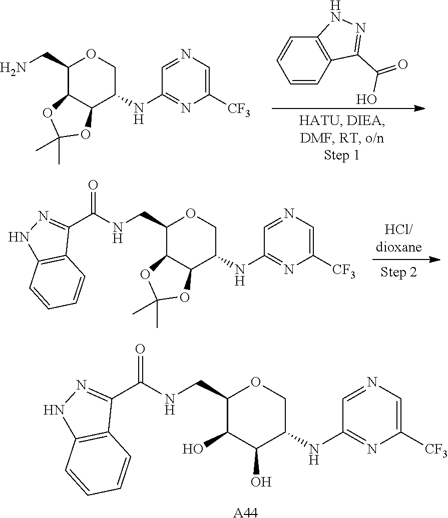

Step 1: A solution of 1H-indazole-3-carboxylic acid (17.86 mg, 0.095 mmol), DIPEA (33.36 mg, 0.259 mmol) and HATU (35.56 mg, 0.095 mmol) in DMF (3 mL) was stirred at an ambient temperature for 30 min. N-((3aR,4R,7S,7aR)-4-(13-amino-2,5,8,11-tetraoxatridecyl)-2,2-dimethyltetrahydro-4H-[1,3]dioxolo[4,5-c]pyran-7-yl)-6-(trifluoromethyl)pyrazin-2-amine (30 mg, 0.087 mmol) was added at an ambient temperature. The reaction was stirred overnight. The resulting mixture was concentrated in vacuo. The crude product was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl) 1H-indazole-3-carboxamide (40 mg, 94.31% yield) as a colorless oil. LC-MS (ESI) found: 493 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-indazole-3-carboxamide (40 mg, 0.081 mmol) in HCl/dioxane (5 mL, 4 N) was stirred at an ambient temperature for 2 h. The resulting mixture was neutralized and the residue was purified by prep-HPLC (0.1% NH₃ H₂O) to give N-(((2R,3R,4R,5S)-3,4-dihydroxy-5-((6-(trifluoromethyl)pyrazin-2-yl)amino)tetrahydro-2H-pyran-2-yl)methyl)-1H-indazole-3-carboxamide (35.2 mg, 95.79% yield) as a white solid. ¹H NMR (400 MHz, CD₃OD): δ 8.22 (d, J=8.2 Hz, 1H), 8.11 (s, 1H), 8.00 (s, 1H), 7.57 (d, J=8.5 Hz, 1H), 7.44-7.39 (m, 1H), 7.28-7.23 (m, 1H), 4.37 (td, J=10.5, 5.1 Hz, 1H), 4.16 (dd, J=11.0, 5.2 Hz, 1H), 3.95 (d, J=3.1 Hz, 1H), 3.79 (dd, J=13.3, 5.2 Hz, 1H), 3.70-3.65 (m, 2H), 3.58 (dd, J=13.4, 7.5 Hz, 1H), 3.15 (t, J=10.9 Hz, 1H). LC-MS (ESI) found: 453 [M+H]⁺.

### Preparation of 3,3′-((2-(6-aminohexanamido)propane-1,3-diyl)bis(oxy))bis(N-(1-((2R,3R,4R,5S)-3,4-dihydroxy-5-((4-(trifluoromethyl)pyrimidin-2-yl)amino)tetrahydro-2H-pyran-2-yl)-2,5,8,11-tetraoxatridecan-13-yl)propanamide) (A45)

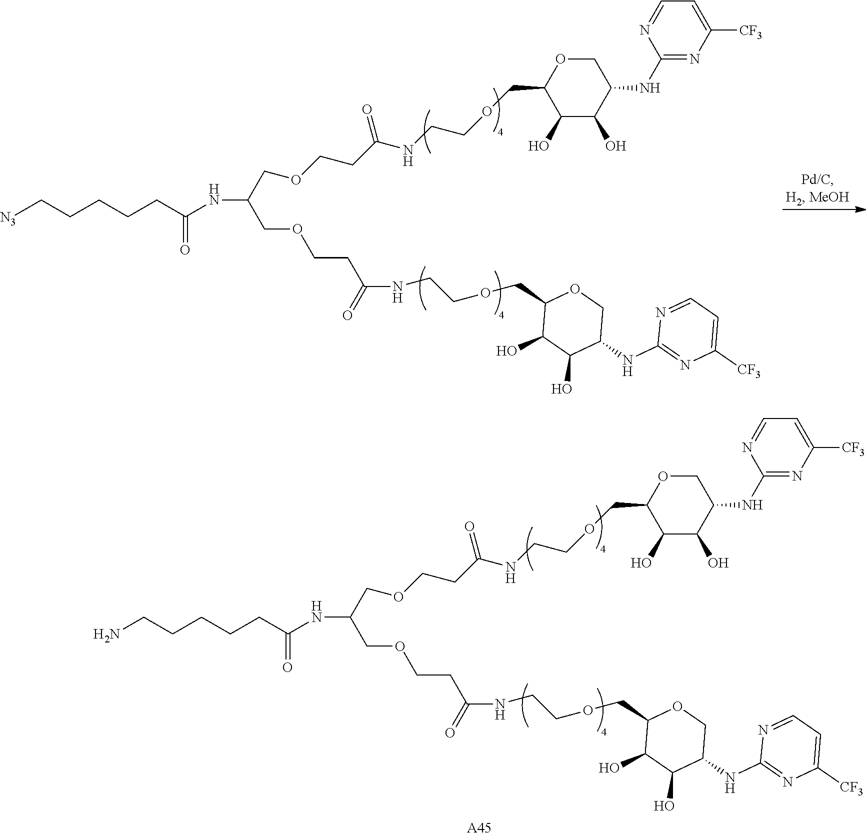

To a solution of 3,3′-((2-(6-azidohexanamido)propane-1,3-diyl)bis(oxy))bis(N-(1-((2R,3R,4R,5S) 3,4-dihydroxy-5-((4-(trifluoromethyl)pyrimidin-2-yl)amino)tetrahydro-2H-pyran-2-yl)-2,5,8,11-tetraoxatridecan-13-yl)propanamide) (300 mg, 0.23 mmol) in MeOH (6 mL) was added Pd/C (30 mg, 10% w) and ammonium hydroxide (0.2 mL) at an ambient temperature. Then the reaction was charged with H2 and stirred at an ambient temperature for 0.5 h. TLC (PE/EA=1/1) showed starting material was consumed and new spot detected. The reaction was filtered and concentrated to give 3,3′-((2-(6-aminohexanamido)propane-1,3-diyl)bis(oxy))bis(N-(1-((2R,3R,4R,5S)-3,4-dihydroxy-5-((4-(trifluoromethyl)pyrimidin-2-yl)amino)tetrahydro-2H-pyran-2-yl)-2,5,8,11-tetraoxatridecan-13-yl)propanamide) (212 mg, 72.3% yield) as a yellow oil. ¹H NMR (400 MHz, CD₃OD) δ 8.51 (d, J=4.8 Hz, 1H), 6.90 (d, J=4.9 Hz, 1H), 4.36 (td, J=10.5, 5.2 Hz, 1H), 4.10 (ddd, J=16.3, 11.2, 5.3 Hz, 2H), 3.92 (d, J=2.9 Hz, 1H), 3.76-3.43 (m, 23H), 3.42-3.32 (m, 2H) 3.23-3.07 (m, 1H), 2.82-2.67 (m, 1H), 2.56-2.38 (m, 2H), 2.31-2.12 (m, 1H), 1.58 (ddd, J=30.4, 15.0, 7.5 Hz, 2H), 1.41-1.31 (m, 1H). LC-MS (ESI) found: 1281 [M+H]⁺.

### Preparation of (2R,3R,4R,5S)-2-((4-(piperidin-4-ylmethyl)piperazin-1-yl)methyl)-5-((6-(trifluoromethyl)pyrazin-2-yl)amino)tetrahydro-2H-pyran-3,4-diol (A46)

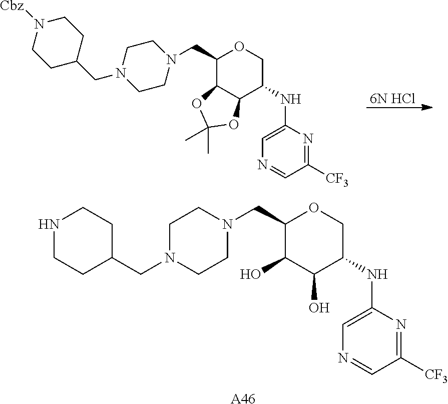

A solution of benzyl 4-((4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)piperazin-1-yl)methyl)piperidine-1-carboxylate (50 mg, 0.1 mmol) in diluted aqueous HCl (4 mL, 6M in water) was stirred at 50° C. overnight. The reaction was cooled and concentrated to give crude mixture. The crude mixture was neutralized with NH₃·H₂O and purified by prep-HPLC to give (2R,3R,4R,5S)-2-((4-(piperidin-4-ylmethyl)piperazin-1-yl)methyl)-5-((6-(trifluoromethyl)pyrazin-2-yl)amino)tetrahydro-2H-pyran-3,4-diol (2.5 mg, 7% yield) as white solid. LC-MS (ESI) found: 475 [M+H]⁺. ¹H NMR (400 MHz, CD₃OD): δ 8.13 (s, 1H), 8.01 (s, 1H), 4.33 (td, J=10.5, 5.1 Hz, 1H), 4.16 (dd, J=11.0, 5.1 Hz, 1H), 3.88 (d, J=2.8 Hz, 1H), 3.84 (d, J=7.4 Hz, 1H), 3.70 (dd, J=10.5, 3.0 Hz, 1H), 3.39 (d, J=12.7 Hz, 2H), 3.28-3.22 (m, 1H), 3.21-3.03 (m, 6H), 2.98 (td, J=12.7, 2.5 Hz, 2H), 2.82-2.54 (m, 4H), 2.36 (d, J=7.2 Hz, 2H), 2.01 (d, J=13.9 Hz, 2H), 1.95-1.85 (m, 1H), 1.43-1.30 (m, 2H).

### Preparation of N-(((2R,3R,4R,5S)-3,4-dihydroxy-5-((6-(trifluoromethyl)pyrazin-2-yl)amino)tetrahydro-2H-pyran-2-yl)methyl)pyridazine-4-carboxamide (Compound A47)

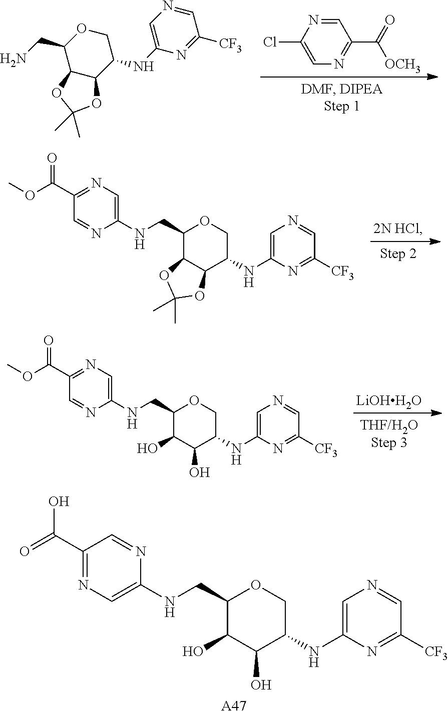

Step 1: To a solution of methyl 5-chloropyrazine-2-carboxylate (37 mg, 0.22 mmol) in DMF (3 mL) were added N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) and DIPEA (37 mg, 0.29 mmol). The mixture was stirred at 100° C. overnight, then concentrated. The residue was purified by flash column to give methyl 5-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)pyrazine-2-carboxylate (60 mg, 88%) as brown oil. LC-MS (ESI) found: 485 [M+H]⁺.
Step 2: To a solution of 5-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)pyrazine-2-carboxylate (60 mg, 0.12 mmol) in THF (3 mL) was added HCl/H₂O (0.5 mL, 2 N) at an ambient temperature. The mixture was stirred at an ambient temperature overnight, then concentrated. The residue was purified by flash chromatography (0˜10% MeOH in DCM) to give methyl 5-((((2R,3R,4R,5S) 3,4-dihydroxy-5-((6-(trifluoromethyl)pyrazin-2-yl)amino)tetrahydro-2H-pyran-2-yl)methyl)amino)pyrazine-2-carboxylate (50 mg, 94%) as white solid. LC-MS (ESI) found: 445 [M+H]⁺.
Step 3: To a solution of 5-((((2R,3R,4R,5S)-3,4-dihydroxy-5-((6-(trifluoromethyl)pyrazin-2-yl)amino)tetrahydro-2H-pyran-2-yl)methyl)amino)pyrazine-2-carboxylate (50 mg, 0.11 mmol) in THF (3 mL) was LiOH·H₂O (5 mg, 0.22 mmol) and water (0.5 mL) at an ambient temperature. The reaction was stirred at an ambient temperature overnight. HCl solution (5 mL, 2 N in H₂O) was added to the reaction to adjust pH to 7, and concentrated. The residue was purified by Prep-HPLC (MeCN/water, 0.1% formic acid) to give 5-((((2R,3R,4R,5S)-3,4-dihydroxy-5-((6-(trifluoromethyl)pyrazin-2-yl)amino)tetrahydro-2H-pyran-2-yl)methyl)amino)pyrazine-2-carboxylic acid (13.4 mg, 28% yield) as white solid. LC-MS (ESI) found: 431 [M+H]⁺. ¹H NMR (400 MHz, CD₃OD): δ 8.67 (s, 1H), 8.10 (s, 1H), 8.00 (s, 1H), 7.96 (s, 1H), 4.36 (td, J=10.6, 5.2 Hz, 1H), 4.14 (dd, J=11.0, 5.1 Hz, 1H), 3.91 (d, J=3.1 Hz, 1H), 3.76 (dd, J=12.7, 3.6 Hz, 1H) 3.70-3.56 (m, 3H), 3.17-3.03 (m, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)oxazole-5-carboxamide (A48)

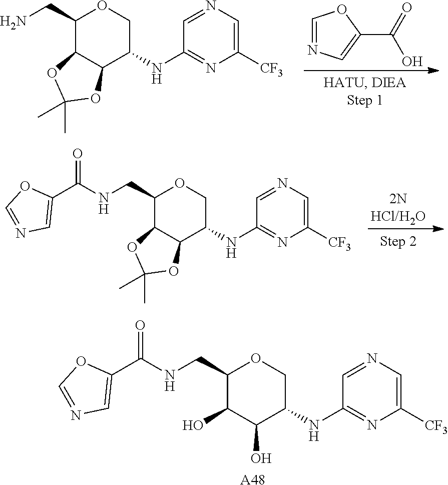

Step 1: To a solution of oxazole-5-carboxylic acid (18 mg, 0.16 mmol) in DMF (3 mL) were added HATU (65 mg, 0.17 mmol) and DIEA (37 mg, 0.29 mmol) at an ambient temperature. After stirring at an ambient temperature for 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The mixture was stirred at an ambient temperature overnight, then concentrated. The residue was purified by flash to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)oxazole-5-carboxamide (60 mg, 96%) as brown oil. LC-MS (ESI) found: 444 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)oxazole-5-carboxamide (60 mg, 0.14 mmol) in THF (3 mL) was added HCl/H₂O (0.5 mL, 2 N) at an ambient temperature. The mixture was stirred at an ambient temperature overnight. Then, the reaction mixture was adjusted the pH=7 with NH₃·H₂O. The residue was purified by Prep-HPLC (MeCN in water from 5% to 70%) to give N-(((2R,3R,4R,5S)-3,4-dihydroxy-5-((6-(trifluoromethyl)pyrazin-2-yl)amino)tetrahydro-2H-pyran-2-yl)methyl)oxazole-5-carboxamide (5.9 mg, 11%) as white solid. ¹H NMR (400 MHz, CD₃OD): δ 8.32 (s, 1H), 8.10 (s, 1H), 8.00 (s, 1H), 7.73 (s, 1H), 4.34 (td, J=10.6, 5.1 Hz, 1H), 4.13 (dd, J=11.0, 5.2 Hz, 1H), 3.89 (d, J=3.2 Hz, 1H), 3.75-3.59 (m, 3H) 3.52 (dd, J=13.2, 7.6 Hz, 1H), 3.11 (t, J=10.8 Hz, 1H). LC-MS (ESI) found: 404 [M+H]⁺.

TABLE 2
Examples in table 2 were prepared following analogous method as that of compound A48

| | | LC-MS | |
|---|---|---|---|
| | | (ESI) | |
| Cmpd | structure | [M + H]⁺ | ¹H-NMR |
| A49 | 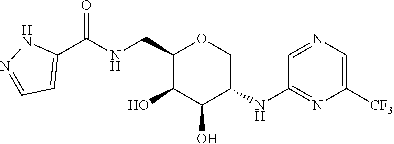 [SMILES: O=C(NC[C@H]1OC[C@H](Nc2cncc(C(F)(F)F)n2)[C@@H](O)[C@H]1O)c1ccn[nH]1] | 403 [M + H]⁺ | ¹H NMR (400 MHz, CD₃OD): δ 8.10 (s, 1H), 7.99 (s, 1H), 7.69 (s, 1H), 6.76 (d, J = 2.0 Hz, 1H), 4.35 (td, J = 10.6, 5.1 Hz, 1H), 4.14 (dd, J = 11.0, 5.2 Hz, 1H), 3.90 (d, J = 3.0 Hz, 1H), 3.74- 3.60 (m, 3H), 3.51 (dd, J = 13.4, 7.6 Hz, 1H), 3.12 (t, J = 10.9 Hz, 1H). |
| A50 | 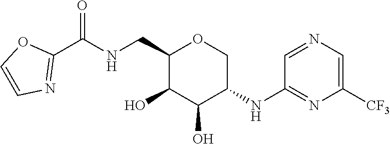 [SMILES: O=C(NC[C@H]1OC[C@H](Nc2cncc(C(F)(F)F)n2)[C@@H](O)[C@H]1O)c1ncco1] | 404 [M + H]⁺ | ¹H NMR (400 MHz, CD₃OD) δ 8.10 (s, 1H), 8.07 (s, 1H), 8.00 (s, 1H), 7.34 (s, 1H), 4.34 (td, J = 10.6, 5.2 Hz, 1H), 4.13 (dd, J = 11.1, 5.2 Hz, 1H), 3.90 (d, J = 3.0 Hz, 1H), 3.72-3.63 (m, 3H), 3.56 (dd, J = 12.7, 7.2 Hz, 1H), 3.12 (t, J = 10.9 Hz, 1H) |
| A52 | 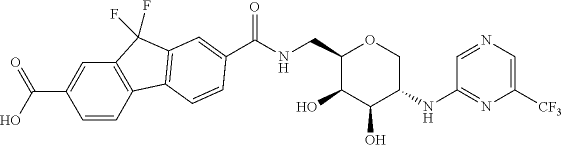 [SMILES: O=C(O)c1ccc2c(c1)C(F)(F)c1cc(C(=O)NC[C@H]3OC[C@H](Nc4cncc(C(F)(F)F)n4)[C@@H](O)[C@H]3O)ccc1-2] | 581 [M + H]⁺ | ¹H NMR (400 MHz, CD₃OD) δ 8.28-8.22 (m, 2H), 8.15 (s, 1H), 8.12-8.05 (m, 2H), 8.00 (s, 1H), 7.91 (dd, J = 7.9, 3.1 Hz, 2H), 4.37 (td, J = 10.6, 5.1 Hz, 1H), 4.16 (dd, J = 11.1, 5.2 Hz, 1H), 3.93 (d, J = 3.0 Hz, 1H), 3.75 (dd, J = 13.1, 4.7 Hz, 1H), 3.71-3.65 (m, 2H), 3.56 (dd, |
| | | | J = 13.1, 7.4 Hz, 1H), 3.14 (t, |
| | | | J = 10.9 Hz, 1H) |

### Preparation of (2R,3R,4R,5S)-2-((2-(2-(2-aminoethoxy)ethoxy)ethoxy)methyl)-5-((6-(trifluoromethyl)pyrazin-2-yl)amino)tetrahydro-2H-pyran-3,4-diol (A53)

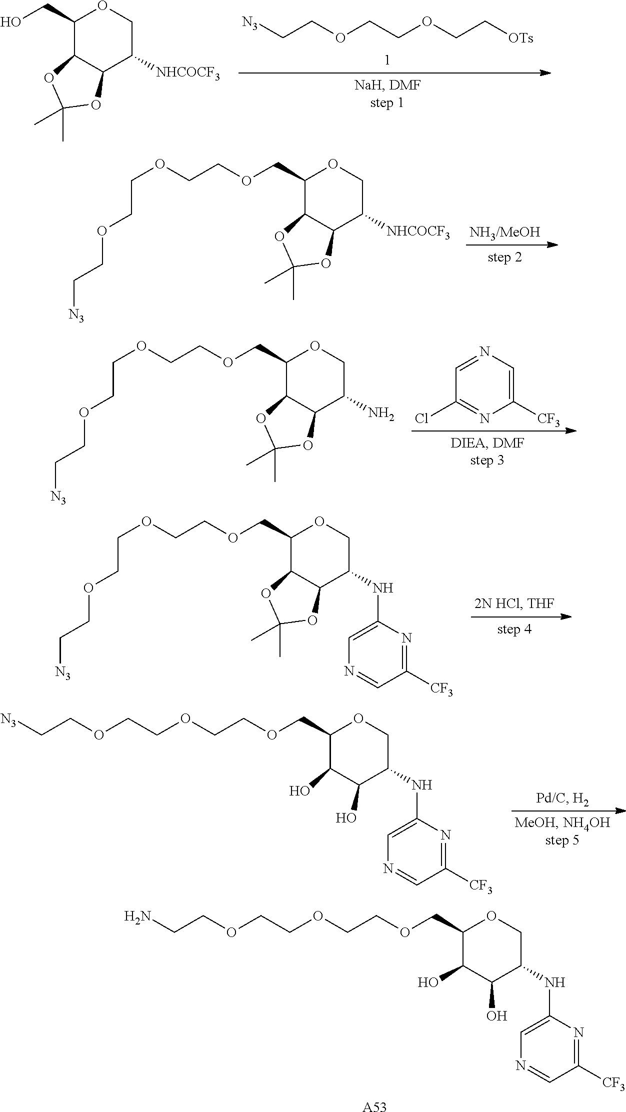

Step 1: To a solution of 2,2,2-trifluoro-N-((3aR,4R,7S,7aR)-4-(hydroxymethyl)-2,2-dimethyltetrahydro-4H-[1,3]dioxolo[4,5-c]pyran-7-yl)acetamide (2.2 g, 7.4 mmol) in DMF (20 mL) was added NaH (0.59 g, 14.7 mmol) at 0° C. After stirring at 0° C. for 20 min, 2-(2-(2-azidoethoxy)ethoxy)ethyl 4-methylbenzenesulfonate (3.6 g, 11 mmol) was added. The mixture was stirred at an ambient temperature overnight. Then the reaction was diluted with H₂O, and extracted with EA. The organic layer was washed with brine, dried over Na₂SO₄, filtered and concentrated. The residue was purified by flash to give N-((3aR,4R,7S,7aR)-4-((2-(2-(2-azidoethoxy)ethoxy)ethoxy)methyl)-2,2-dimethyltetrahydro-4H-[1,3]dioxolo[4,5-c]pyran-7-yl) 2,2,2-trifluoroacetamide (2.5 g, 75%) as colorless oil. LC-MS (ESI) found: 457 [M+H]⁺.
Step 2: A solution of N-((3aR,4R,7S,7aR)-4-((2-(2-(2-azidoethoxy)ethoxy)ethoxy)methyl)-2,2-dimethyltetrahydro-4H-[1,3]dioxolo[4,5-c]pyran-7-yl)-2,2,2-trifluoroacetamide (2.5 g, 5.5 mmol) in NH₃·MeOH (15 mL) was stirred at 80° C. overnight. The mixture was concentrated to give (3aR,4R,7S,7aR)-4-((2-(2-(2-azidoethoxy)ethoxy)ethoxy)methyl)-2,2-dimethyltetrahydro-4H-[1,3]dioxolo[4,5-c]pyran-7-amine (1.6 g, 81%) as colorless oil. LC-MS (ESI) found: 361 [M+H]⁺.
Step 3: A solution of (3aR,4R,7S,7aR)-4-((2-(2-(2-azidoethoxy)ethoxy)ethoxy)methyl)-2,2-dimethyltetrahydro-4H-[1,3]dioxolo[4,5-c]pyran-7-amine (1.6 g, 4.4 mmol), 2-chloro-6-(trifluoromethyl)pyrazine (1.2 g, 6.7 mmol) and DIPEA (1.2 g, 8.9 mmol) in DMF (10 mL) was stirred at 100° C. overnight. The mixture was concentrated and the residue was purified by flash to give N-((3aR,4R,7S,7aR)-4-((2-(2-(2-azidoethoxy)ethoxy)ethoxy)methyl)-2,2-dimethyltetrahydro-4H-[1,3]dioxolo[4,5-c]pyran-7-yl)-6-(trifluoromethyl)pyrazin-2-amine (900 mg, 40%) as colorless oil. LC-MS (ESI) found: 507 [M+H]⁺.
Step 4: To a solution of N-((3aR,4R,7S,7aR)-4-((2-(2-(2-azidoethoxy)ethoxy)ethoxy)methyl) 2,2-dimethyltetrahydro-4H-[1,3]dioxolo[4,5-c]pyran-7-yl)-6-(trifluoromethyl)pyrazin-2-amine (900 mg, 1.8 mmol) in THF (3 mL) was added HCl/H₂O (2.0 N, 1 mL). The mixture was stirred at an ambient temperature overnight, then concentrated. The residue was purified by flash to give (2R,3R,4R,5S)-2-((2-(2-(2-azidoethoxy)ethoxy)ethoxy)methyl)-5-((6-(trifluoromethyl)pyrazin-2-yl)amino)tetrahydro-2H-pyran-3,4-diol (500 mg, 60%) as brown oil. LC-MS (ESI) found: 467 [M+H]⁺.
Step 5: To a solution of (2R,3R,4R,5S)-2-((2-(2-(2-azidoethoxy)ethoxy)ethoxy)methyl)-5-((6-(trifluoromethyl)pyrazin-2-yl)amino)tetrahydro-2H-pyran-3,4-diol (500 mg, 1.1 mmol) in MeOH (3 mL) were added Pd/C (114 mg, 1.1 mmol) and NH₄₀H (1 mL). The mixture was stirred at an ambient temperature under H2 for 30 min, then filtered through a Celite pad, and the filtrate was concentrated to give crude product, which was purified by flash to give (2R,3R,4R,5S)-2-((2-(2-(2-aminoethoxy)ethoxy)ethoxy)methyl)-5-((6-(trifluoromethyl)pyrazin-2-yl)amino)tetrahydro-2H-pyran-3,4-diol (230 mg, 48%) as white solid. LC-MS (ESI) found: 441 [M+H]⁺. ¹H NMR (400 MHz, CD₃OD) δ 8.11 (s, 1H), 8.00 (s, 1H), 4.33 (s, 1H), 4.12 (dd, J=11.0, 5.2 Hz, 1H), 3.93 (d, J=2.8 Hz, 1H), 3.74-3.54 (m, 14H), 3.13 (t, J=10.8 Hz, 1H), 2.96-2.87 (m, 2H).

### Preparation of (1R,2S,3R,6S)-3-(hydroxymethyl)-6-((4-(trifluoromethyl)pyrimidin-2-yl)amino)cyclohexane-1,2-diol (A54)

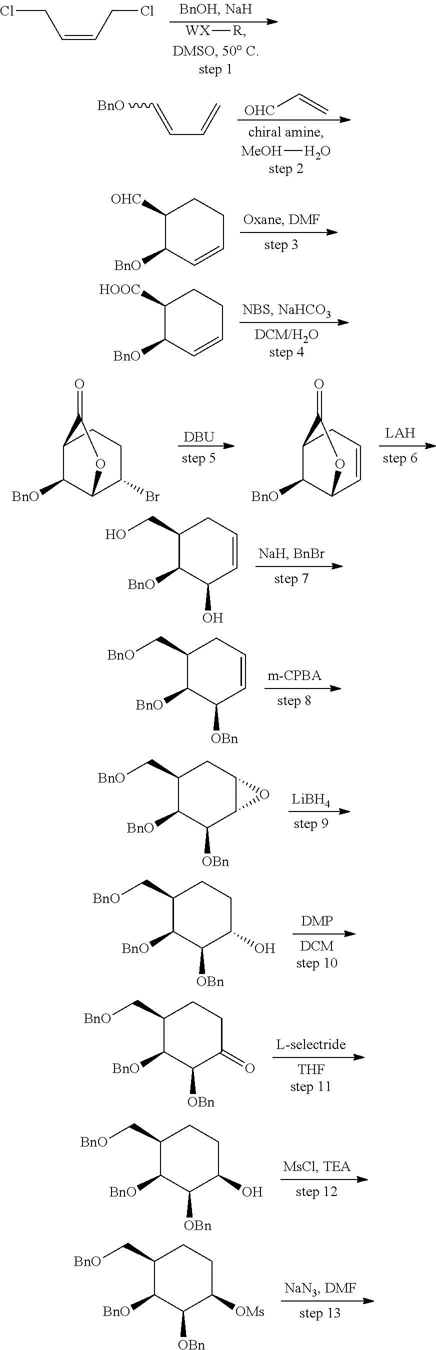

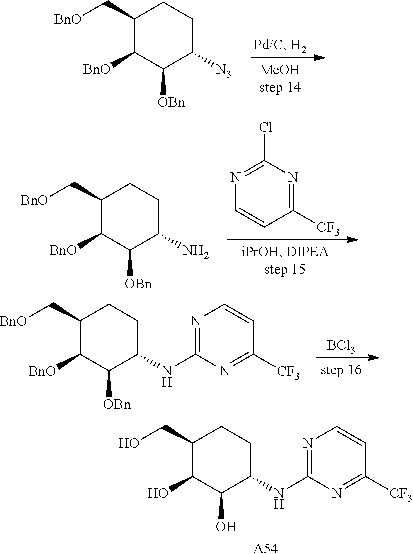

### Step 1

To a suspension of NaH (26 g, 380 mmol, 60% in oil) and 4,4′-Thiobis(6-tert-butyl-m-cresol) (5.0 g, 14 mmol) in dry DMSO (1 L) was added BnOH (52 mL, 500 mmol) dropwise under N₂. After stirring at an ambient temperature for 1 h, (Z)-1,4-dichlorobut-2-ene (22 mL, 200 mmol) was added and the reaction was stirred at 50° C. for 3 h. The mixture was quenched with saturated NH₄C1 and extracted with ethyl acetate. The organic layer was dried over Na₂SO₄, filtered and concentrated. The residue was purified by flash to give ((buta-1,3-dien-1-yloxy)methyl)benzene (20 g, 77%) as yellow oil.

### Step 2

A solution of MacMillan catalyst (3 g, 12.9 mmol) in MeOH (20 mL) and H₂O (6 mL) was cooled to −10° C. Acrylaldehyde (22.5 mL, 340 mmol) and a solution of ((buta-1,3-dien-1-yloxy)methyl)benzene (20 g, 125 mmol) in MeOH (24 mL) were added. After stirring at −10° C. overnight, the mixture was diluted with H₂O. The resulting solution was extracted with ethyl acetate. The organic layer was concentrated, the residue was purified by flash to give (1S,2R)-2-(benzyloxy)cyclohex-3-ene-1-carbaldehyde (13 g, 48%) as yellow oil.

### Step 3

To a solution of (1S,2R)-2-(benzyloxy)cyclohex-3-ene-1-carbaldehyde (13 g, 60 mmol) in DMF (3 mL) was add oxane (55 g, 90 mmol). After stirring at an ambient temperature for 2 h, the mixture was quenched with HCl (2 N). The resulting solution was extracted with EA and the organic layer was concentrated to give (1S,2R)-2-(benzyloxy)cyclohex-3-ene-1-carboxylic acid (9 g, 64%) as colorless oil.

### Step 4

To a solution of (1S,2R)-2-(benzyloxy)cyclohex-3-ene-1-carboxylic acid (9 g, 39 mmol) in DCM (3 mL) was add NaHCO₃ (16 g, 194 mmol) in H₂O (1 mL). After stirring at an ambient temperature for 10 min, NBS (10 g, 58 mmol) was added. The mixture was stirred at an ambient temperature for 2 h, then the reaction was diluted with H₂O and extracted with ethyl acetate. The organic layer was concentrated and the residue was purified by flash chromatography to give (1S,4S,5S,8S)-8-(benzyloxy)-4-bromo-6-oxabicyclo[3.2.1]octan-7-one (6 g, 50%) as colorless oil.

### Step 5

To a solution of (1S,4S,5S,8S)-8-(benzyloxy)-4-bromo-6-oxabicyclo[3.2.1]octan-7-one (6 g, 19 mmol) in THF (50 mL) was added DBU (14 mL, 93 mmol) at an ambient temperature. The mixture was stirred at 50° C. overnight. The solvent was concentrated and the residue was purified by flash chromatography to give (1S,5R,8S)-8-(benzyloxy)-6-oxabicyclo[3.2.1]oct-3-en-7-one (3.3 g, 74%) as white solid.

### Step 6

A solution of (1S,5R,8S)-8-(benzyloxy)-6-oxabicyclo[3.2.1]oct-3-en-7-one (3.3 g, 14 mmol) in Et₂O (15 mL) was cooled at −78° C., and LAH (11 mL) (1 N in Et₂O) was added dropwise under N2. The mixture was stirred at −20° C. for 1.5 h. To the mixture were added acetone (5 mL) and saturated Rochelle salt (5 mL). The mixture was stirred at an ambient temperature for 1 h, then diluted with H₂O, and extracted with ethyl acetate. The organic layer was concentrated and the residue was purified by flash to give (1R,5R,6S)-6-(benzyloxy)-5-(hydroxymethyl)cyclohex-2-en-1-01 (2.8 g, 84%) as yellow oil.

### Step 7

To a solution of (1R,5R,6S)-6-(benzyloxy)-5-(hydroxymethyl)cyclohex-2-en-1-ol (2.8 g, 12 mmol) in DMF (5 mL) was added NaH (1.4 g, 36 mmol) at 0° C. After stirring at 0° C. for 20 min, BnBr (8 g, 48 mmol) was added. The mixture was stirred at an ambient temperature overnight, then diluted with H₂O, and extracted with ethyl acetate. The organic layer was concentrated and the residue was purified by flash chromatography to give ((((1S,2R,6R)-6-((benzyloxy)methyl)cyclohex-3-ene-1,2-diyl)bis(oxy))bis(methylene))dibenzene (3.4 g, 68%) as yellow oil. LC-MS (ESI) found: 437 [M+Na]⁺.

### Step 8

To a solution of ((((1S,2R,6R)-6-((benzyloxy)methyl)cyclohex-3-ene-1,2-diyl)bis(oxy))bis(methylene))dibenzene (3 g, 7.2 mmol) in DCM (2 mL) was added m-CPBA (6.2 g, 36 mmol) at 0° C. The mixture was diluted with DCM, washed with saturated NaHCO₃, and the organic layer was concentrated. The residue was purified by flash chromatography to give (1S,2S,3S,4R,6S)-2,3-bis(benzyloxy)-4-((benzyloxy)methyl)-7-oxabicyclo[4.1.0]heptane (1.9 g, 60.98%) as white solid. LC-MS (ESI) found: 453 [M+Na]⁺.

### Step 9

To a solution of (1S,2S,3S,4R,6S)-2,3-bis(benzyloxy)-4-((benzyloxy)methyl)-7-oxabicyclo[4.1.0]heptane (1.7 g, 3.9 mmol) in THF (20 mL) was added LiBH₄ (20 mL, 39 mmol) at 0° C. The mixture was stirred at 80° C. for 2 days, and the reaction was quenched with Na₂SO₄·10H₂O. The solid was filtered off and washed with THF. The organic layer was concentrated and the residue was purified by flash chromatography to give (1S,2R,3S,4R)-2,3-bis(benzyloxy)-4-((benzyloxy)methyl)cyclohexan-1-ol (780 mg, 46%) as colorless oil. LC-MS (ESI) found: 455 [M+Na]⁺.

### Step 10

To a solution of (1S,2R,3S,4R)-2,3-bis(benzyloxy)-4-((benzyloxy)methyl)cyclohexan-1-ol (500 mg, 1.15 mmol) in DCM (3 mL) was added Dess-Martin (735 mg, 1.73 mmol) at 0° C. The mixture was stirred at an ambient temperature for 1 h, then quenched with saturated NaHCO₃. The mixture was extracted with DCM, the organic layer was concentrated, and the residue was purified by flash chromatography to give (2S,3S,4R)-2,3-bis(benzyloxy)-4-((benzyloxy)methyl)cyclohexan-1-one (360 mg, 72%) as colorless oil. LC-MS (ESI) found: 453 [M+Na]⁺.

### Step 11

To a solution of (2S,3S,4R)-2,3-bis(benzyloxy)-4-[(benzyloxy)methyl]cyclohexan-1-one (360 mg, 0.84 mmol) in THF (2 mL) was added L-selectride (8.3 mL) at −78° C. The mixture was stirred at an ambient temperature for 2 h, then quenched with MeOH. The organic layer was concentrated and the residue was purified by flash chromatography to give (1R,2R,3S,4R)-2,3-bis(benzyloxy) 4-((benzyloxy)methyl)cyclohexan-1-ol (300 mg, 83%) as colorless oil. LC-MS (ESI) found: 455 [M+Na]⁺.

### Step 12

To a solution of (1R,2R,3S,4R)-2,3-bis(benzyloxy)-4-((benzyloxy)methyl)cyclohexan-1-ol (200 mg, 0.46 mmol) in DCM (2 mL) were added TEA (0.26 mL, 0.92 mmol) and MsCl (0.1 mL, 0.92 mmol) at 0° C. The mixture was stirred at an ambient temperature for 3 h. Then the reaction was diluted and extracted with DCM. The organic layer was concentrated to give (1R,2S,3S,4R)-2,3-bis(benzyloxy)-4-((benzyloxy)methyl)cyclohexyl methanesulfonate (180 mg, 76%) as yellow oil. LC-MS (ESI) found: 511 [M+H]⁺.

### Step 13

A solution of (1R,2S,3S,4R)-2,3-bis(benzyloxy)-4-((benzyloxy)methyl)cyclohexyl methanesulfonate (180 mg, 0.35 mmol) and NaN₃ (229 mg, 3.5 mmol) in DMF (2 mL) was stirred at 100° C. overnight. The mixture was diluted with H₂O, extracted with ethyl acetate, and the organic layer was concentrated. The residue was purified by prep-TLC to give ((((1S,2R,3S,6R) 3-azido-6-((benzyloxy)methyl)cyclohexane-1,2-diyl)bis(oxy))bis(methylene))dibenzene (30 mg, 19%) as colorless oil. LC-MS (ESI) found: 480 [M+Na]*.

### Step 14

A solution of ((((1S,2R,3S,6R)-3-azido-6-((benzyloxy)methyl)cyclohexane-1,2-diyl)bis(oxy))bis(methylene))dibenzene (30 mg, 0.07 mmol), Pd/C (10 mg, 10% wt, 60% wet) in MeOH (2 mL) was stirred at an ambient temperature under H2 balloon for 30 min. The mixture was filtered through a Celite pad, and the filtrate was concentrated to give crude product (1S,2R,3S,4R)-2,3-bis(benzyloxy)-4-((benzyloxy)methyl)cyclohexan-1-amine (20 mg, 71%) as colorless oil. LC-MS (ESI) found: 432 [M+H]⁺.

### Step 15

A solution of ((((1S,2R,3S,6R)-3-azido-6-((benzyloxy)methyl)cyclohexane-1,2-diyl)bis(oxy))bis(methylene))dibenzene (20 mg, 0.05 mmol), 2-chloro-4-(trifluoromethyl)pyrimidine (12 mg, 0.07 mmol) and DIPEA (15 mg, 0.12 mmol) in i-PrOH (5 mL) was stirred at 80° C. under N₂ balloon overnight. The mixture was concentrated and the mixture was purified by flash chromatography to give N-((1S,2R,3S,4R)-2,3-bis(benzyloxy)-4-((benzyloxy)methyl)cyclohexyl)-4-(trifluoromethyl)pyrimidin-2-amine (15 mg, 58%) as colorless oil. LC-MS (ESI) found: 578[M+H]⁺.

### Step 16

To a solution of N-((1S,2R,3S,4R)-2,3-bis(benzyloxy)-4-((benzyloxy)methyl)cyclohexyl)-4-(trifluoromethyl)pyrimidin-2-amine (15 mg, 0.03 mmol) in DCM (2 mL) was added BCl₃ (0.5 mL) at 0° C. The mixture was stirred at an ambient temperature overnight, then quenched with NH₄₀H. The residue was purified by flash chromatography to give (1R,2S,3R,6S)-3-(hydroxymethyl)-6-((4-(trifluoromethyl)pyrimidin-2-yl)amino)cyclohexane-1,2-diol (3 mg, 37%) as white solid. LC-MS (ESI) found: 308[M+H]⁺. ¹H NMR (400 MHz, CD₃OD): δ 8.48 (d, J=4.9 Hz, 1H), 6.84 (d, J=4.9 Hz, 1H), 4.21 (td, J=11.3, 4.5 Hz, 1H), 4.07 (t, J=2.3 Hz, 1H), 3.65 (dd, J=10.6, 7.2 Hz, 1H), 3.49 (ddd, J=13.1, 10.5, 4.4 Hz, 2H), 2.08 (dd, J=12.7, 4.1 Hz, 1H), 1.67-1.58 (m, 1H), 1.56-1.44 (m, 2H), 1.33 (ddd, J=24.2, 12.4, 5.0 Hz, 1H).

### Preparation of (2R,3R,4R,5S)-2-(((1-(pyrimidin-2-yl)ethyl)amino)methyl)-5-((6-(trifluoromethyl)pyrazin-2-yl)amino)tetrahydro-2H-pyran-3,4-diol(A55), (2R,3R,4R,5S)-2-((((R)-1-(pyrimidin-2-yl)ethyl)amino)methyl)-5-((6-(trifluoromethyl)pyrazin-2-yl)amino)tetrahydro-2H-pyran-3,4-diol (A58) and (2R,3R,4R,5S)-2-((((S)-1-(pyrimidin-2-yl)ethyl)amino)methyl)-5-((6-(trifluoromethyl)pyrazin-2-yl)amino)tetrahydro-2H-pyran-3,4-diol (A59)

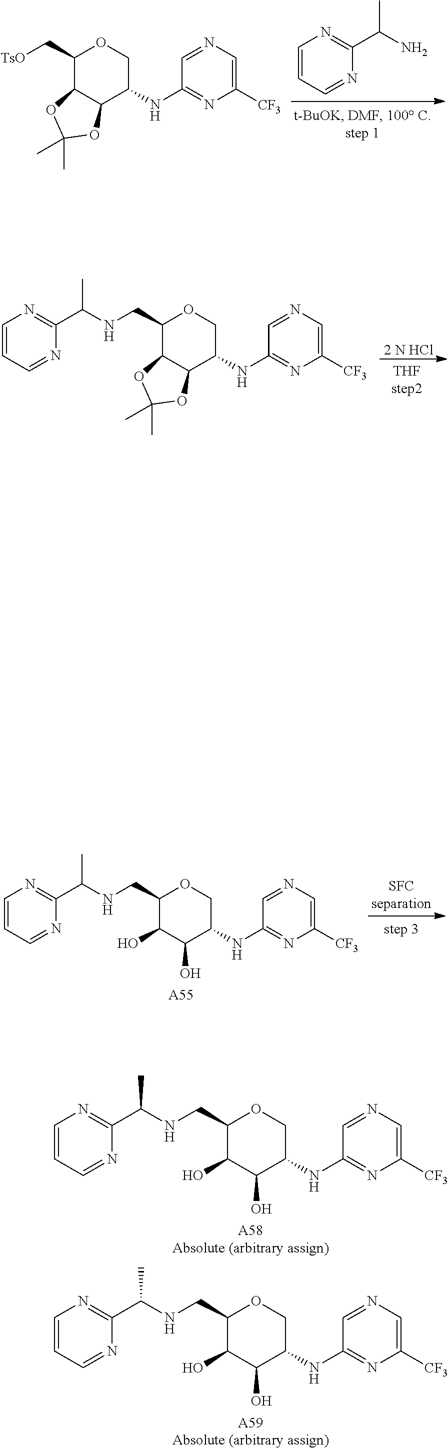

Step 1: A solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (500 mg, 1.08 mmol), t-BuOK (2 mL, 2 mmol) and 1-(pyrimidin-2-yl)ethan-1-amine (159 mg, 1.3 mmol) in DMF (6 mL) was stirred at 100° C. for 18 h. The resulting mixture was concentrated in vacuo. The crude product was purified by flash chromatography (silica gel, 0˜10% MeOH in DCM) to give N-((3aS,4R,7S,7aR)-2,2-dimethyl-4-(((1-(pyrimidin-2-yl)ethyl)amino)methyl)tetrahydro-4H-[1,3]dioxolo[4,5-c]pyran-7-yl)-6-(trifluoromethyl)pyrazin-2-amine (120 mg, yield: 26.8%) as a brown solid. LC-MS (ESI) found: 455 [M+H]⁺.
Step 2: To a solution of N-((3aS,4R,7S,7aR)-2,2-dimethyl-4-(((1-(pyrimidin-2-yl)ethyl)amino)methyl)tetrahydro-4H-[1,3]dioxolo[4,5-c]pyran-7-yl)-6-(trifluoromethyl)pyrazin-2-amine (120 mg, 0.26 mmol) in THF (4 mL) was added HCl (2 mL, 4 mmol). The reaction was stirred at an ambient temperature for 1 hr. The resulting mixture was filtered and concentrated in vacuo. The reaction was neutralized with saturated sodium bicarbonate. The crude product was purified by prep-HPLC to give (2R,3R,4R,5S)-2-(((1-(pyrimidin-2-yl)ethyl)amino)methyl)-5-((6-(trifluoromethyl)pyrazin-2-yl)amino)tetrahydro-2H-pyran-3,4-diol (60 mg, yield: 54.8%) as a white solid. LC-MS (ESI) found: 415 [M+H]⁺. ¹H NMR (400 MHz, CD₃OD): δ 8.82 (d, J=4.9 Hz, 2H), 8.12 (s, 1H), 8.01 (s, 1H), 7.42 (td, J=4.9, 1.7 Hz, 1H), 4.36-4.21 (m, 2H), 4.16 (ddd, J=11.0, 5.1, 1.6 Hz, 1H), 3.88 (dd, J=12.5, 2.5 Hz, 1H), 3.66 (ddd, J=11.7, 5.6, 2.6 Hz, 2H) 3.17-3.00 (m, 2H), 2.93 (dd, J=12.6, 3.2 Hz, 1H), 1.55 (dd, J=6.8, 3.7 Hz, 3H). ¹⁹F NMR (377 MHz, CD₃OD): δ −70.32 (s).
Step 3: The compound of (2R,3R,4R,5S)-2-(((1-(pyrimidin-2-yl)ethyl)amino)methyl)-5-((6-(trifluoromethyl)pyrazin-2-yl)amino)tetrahydro-2H-pyran-3,4-diol (40 mg) was separated by SFC Column: ChiralCel OZ, 250×21.2 mm I.D., 5 μm. Mobile phase: A for CO2 and B for MeOH+0.1% NH₃·H₂O). Obtained absolute (arbitrary assign) (2R,3R,4R,5S)-2-((((R)-1-(pyrimidin-2-yl)ethyl)amino)methyl)-5-((6-(trifluoromethyl)pyrazin-2-yl)amino)tetrahydro-2H-pyran-3,4-diol(18 mg, yield: 45%). ¹H NMR (400 MHz, CD₃OD) δ 8.79 (d, J=4.9 Hz, 2H), 8.10 (s, 1H), 7.99 (s, 1H), 7.38 (t, J=4.9 Hz, 1H), 4.29 (td, J=10.6, 5.2 Hz, 1H), 4.12 (dd, J=11.0, 5.2 Hz, 1H), 4.03 (q, J=6.8 Hz, 1H), 3.82 (d, J=2.9 Hz, 1H), 3.64 (dd, J=10.5, 3.2 Hz, 1H) 3.55 (dd, J=8.4, 2.8 Hz, 1H), 3.10 (t, J=10.8 Hz, 1H), 2.84 (dd, J=12.3, 8.4 Hz, 1H), 2.71 (dd, J=12.3, 3.3 Hz, 1H), 1.45 (d, J=6.8 Hz, 3H). LC-MS (ESI) found: 415 [M+H]⁺. Obtained absolute (arbitrary assign) (2R,3R,4R,5S)-2-((((S)-1-(pyrimidin-2-yl)ethyl)amino)methyl)-5-((6-(trifluoromethyl)pyrazin-2-yl)amino)tetrahydro-2H-pyran-3,4-diol (16 mg, yield: 40%). LC-MS (ESI) found: 415 [M+H]⁺. ¹H NMR (400 MHz, CD₃OD) δ 8.84 (d, J=5.0 Hz, 2H), 8.12 (s, 1H) 8.01 (s, 1H), 7.45 (t, J=4.9 Hz, 1H), 4.45-4.29 (m, 2H), 4.18 (dd, J=11.0, 5.1 Hz, 1H), 3.90 (d, J=2.9 Hz, 1H), 3.69 (dd, J=15.9, 5.2 Hz, 2H), 3.29-3.23 (m, 1H), 3.15 (t, J=10.8 Hz, 1H) 3.05 (dd, J=12.7, 3.3 Hz, 1H), 1.61 (d, J=6.9 Hz, 3H).

### Preparation of (2R,3R,4R,5S)-2-((2-(2-(pyrazin-2-ylamino)ethoxy)ethoxy)methyl)-5-((6-(trifluoromethyl)pyrazin-2-yl)amino)tetrahydro-2H-pyran-3,4-diol (A56)

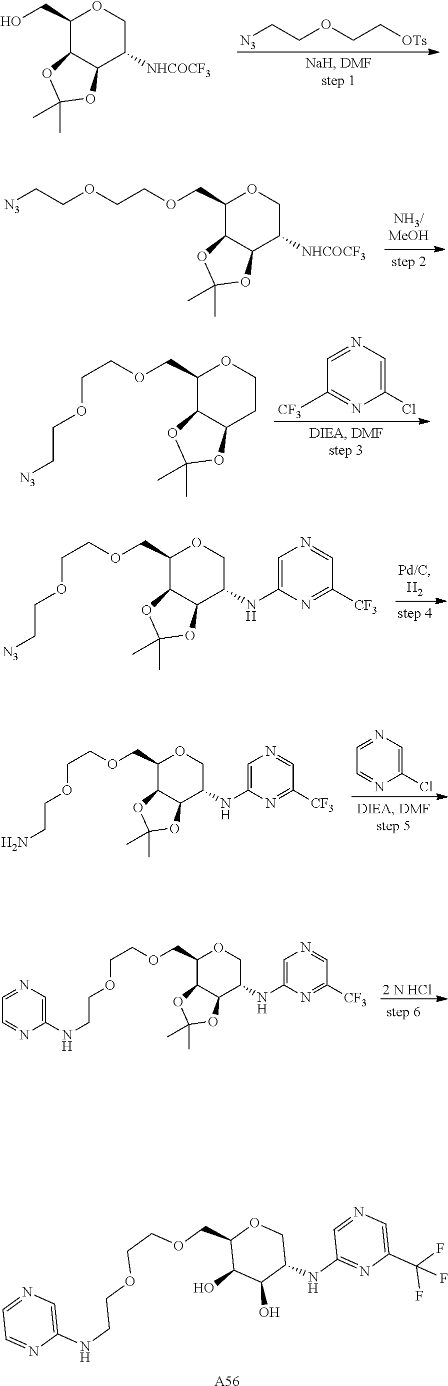

Step 1: To a mixture of NaH (240 mg, 6.0 mmol) in anhydrous DMF (5 mL) was added 2,2,2-trifluoro-N-((3aR,4R,7S,7aR)-4-(hydroxymethyl)-2,2-dimethyltetrahydro-4H-[1,3]dioxolo[4,5-c]pyran-7-yl)acetamide (598 mg, 2.0 mmol) under argon at 0° C. The mixture was stirred at 0° C. under argon for 1 h. Then 2-(2-azidoethoxy)ethyl 4-methylbenzenesulfonate (852 mg, 3.0 mmol) was added dropwise to the above reaction mixture at 0° C. under argon. The mixture was stirred at an ambient temperature overnight. The reaction was quenched with ice-water (5 mL) at 0° C. The reaction mixture was extracted with ethyl acetate (3 mL×3). The organic layers were washed with saturated NaCl, dried over Na₂SO₄, filtered and concentrated. The crude was purified by flash silica column chromatography (eluent=50% MeOH in DCM) to give N-((3aR,4R,7S,7aR)-4-((2-(2-azidoethoxy)ethoxy)methyl)-2,2-dimethyltetrahydro-4H-[1,3]dioxolo[4,5-c]pyran-7-yl)-2,2,2-trifluoroacetamide (500 mg, yield: 60.67%) as yellow oil. LC-MS (ESI) found: 413 [M+H]⁺.
Step 2: A solution of N-((3aR,4R,7S,7aR)-4-((2-(2-azidoethoxy)ethoxy)methyl)-2,2-dimethyltetrahydro-4H-[1,3]dioxolo[4,5-c]pyran-7-yl)-2,2,2-trifluoroacetamide (500 mg, 1.21 mmol) in ammonia (10 mL, 7.0 M in methanol) was stirred at 80° C. overnight. The mixture was concentrated in vacuo to give (3aR,4R,7S,7aR)-4-((2-(2-azidoethoxy)ethoxy)methyl)-2,2-dimethyltetrahydro-4H-[1,3]dioxolo[4,5-c]pyran-7-amine (350 mg, yield: 90%) as yellow oil. LC-MS (ESI) found: 317 [M+H]⁺.
Step 3: To a solution of (3aR,4R,7S,7aR)-4-((2-(2-azidoethoxy)ethoxy)methyl)-2,2-dimethyltetrahydro-4H-[1,3]dioxolo[4,5-c]pyran-7-amine (350 mg, 1.10 mmol) in DMF (5 mL) were added DIEA (350 mg, 2.71 mmol) and 2-chloro-6-(trifluoromethyl)pyrazine (360 mg, 1.97 mmol). The mixture was stirred at an ambient temperature under argon overnight. The reaction mixture was diluted water (5 mL). The reaction mixture was extracted with ethyl acetate (5 mL×3). The organic layers were washed with brine, dried by Na₂SO₄, filtered and concentrated. The crude was purified by flash silica column chromatography (eluent=50% EA in PE) to give N-((3aR,4R,7S,7aR)-4-((2-(2-azidoethoxy)ethoxy)methyl)-2,2-dimethyltetrahydro-4H-[1,3]dioxolo[4,5-c]pyran-7-yl)-6-(trifluoromethyl)pyrazin-2-amine (130 mg, yield: 25%) as yellow oil. LC-MS (ESI) found: 463 [M+H]⁺.
Step 4: A solution of N-((3aR,4R,7S,7aR)-4-((2-(2-azidoethoxy)ethoxy)methyl)-2,2-dimethyltetrahydro-4H-[1,3]dioxolo[4,5-c]pyran-7-yl)-6-(trifluoromethyl)pyrazin-2-amine (130 mg, 0.28 mmol) in MeOH (10 mL) was stirred at an ambient temperature under a balloon of H2 for 30 min. The mixture was filtered through a pad of celite, the filtrate was concentrated in vacuo to give N-((3aR,4R,7S,7aR)-4-((2-(2-aminoethoxy)ethoxy)methyl)-2,2-dimethyltetrahydro-4H-[1,3]dioxolo[4,5-c]pyran-7-yl)-6-(trifluoromethyl)pyrazin-2-amine (100 mg, yield: 81%) as yellow oil. LC-MS (ESI) found: 437 [M+H]⁺.
Step 5: To a solution of N-((3aR,4R,7S,7aR)-4-((2-(2-aminoethoxy)ethoxy)methyl)-2,2-dimethyltetrahydro-4H-[1,3]dioxolo[4,5-c]pyran-7-yl)-6-(trifluoromethyl)pyrazin-2-amine (100 mg, 0.22 mmol) in DMF (5 mL) were added DIEA (88 mg, 0.69 mmol) and 2-chloropyrazine (50 mg, 0.43 mmol). The mixture was stirred at 100° C. under argon overnight. The reaction mixture was cooled to rt and quenched with water (5 mL). The mixture was extracted with ethyl acetate (3 mL×3). The combined organic layers were washed with saturated NaCl solution (5 mL), dried by Na₂SO₄, filtered and concentrated. The residue was purified by flash silica column chromatography (eluent=8% MeOH in DCM) to give N-((3aR,4R,7S,7aR)-2,2-dimethyl-4-((2-(2-(pyrazin-2-ylamino)ethoxy)ethoxy)methyl)tetrahydro-4H-[1,3]dioxolo[4,5-c]pyran-7-yl)-6-(trifluoromethyl)pyrazin-2-amine (100 mg, yield: 85%) as a yellow oil. LC-MS (ESI) found: 515 [M+H]⁺.
Step 6: To a solution of N-((3aR,4R,7S,7aR)-2,2-dimethyl-4-((2-(2-(pyrazin-2-ylamino)ethoxy)ethoxy)methyl)tetrahydro-4H-[1,3]dioxolo[4,5-c]pyran-7-yl)-6-(trifluoromethyl)pyrazin-2-amine (100 mg, 0.19 mmol) in THF (2 ml) was added 2 N HCl (1 mL), then the mixture was stirred at an ambient temperature for 1 h. The pH of the reaction mixture was adjusted to 7 with NaHCO₃ and concentrated. The residue was purified by Prep-HPLC (MeCN/H₂O, 0.1% FA) to give N-(((2R,3R,4R,5 S)-3,4-dihydroxy-5-((6-(trifluoromethyl)pyrazin-2-yl)amino)tetrahydro-2H-pyran-2-yl)methyl)quinoxaline-2-carboxamide as yellow solid (23 mg, yield: 25%). LC-MS (ESI) found: 475 [M+H]⁺. ¹H NMR (400 MHz, CD₃OD): δ 8.11 (s, 1H), 8.00 (s, 1H), 7.93 (q, J=1.5 Hz, 2H), 7.62 (d, J=2.7 Hz, 1H), 4.34 (td, J=10.6, 5.2 Hz, 1H), 4.16-4.08 (m, 1H), 3.91 (d, J=2.9 Hz, 1H), 3.73-3.65 (m, 8H), 3.64-3.62 (m, 1H), 3.61-3.57 (m, 1H) 3.53 (t, J=5.4 Hz, 2H), 3.11 (t, J=10.8 Hz, 1H).

### Preparation of 2-(2-(2-aminoethoxy)ethoxy)-N-(((2R,3R,4R,5S)-3,4-dihydroxy-5-((6-(trifluoromethyl)pyrazin-2-yl)amino)tetrahydro-2H-pyran-2-yl)methyl)acetamide (A57)

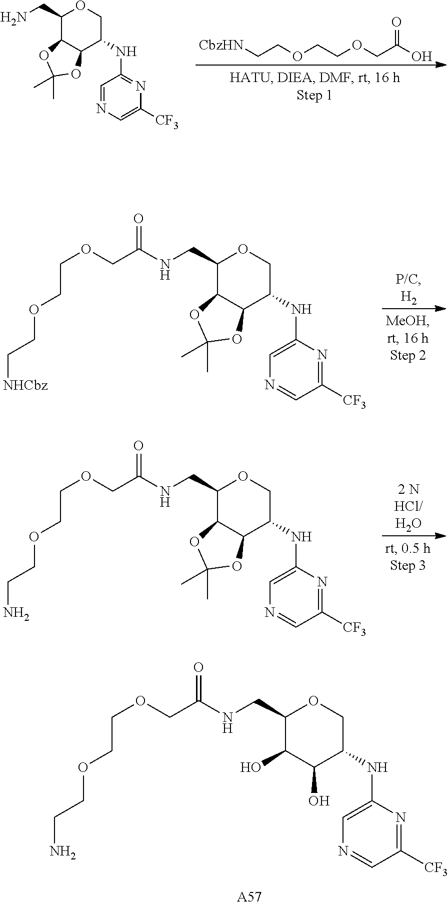 [SMILES: CC1(C)O[C@@H]2[C@H](O1)[C@@H](Nc1cncc(C(F)(F)F)n1)CO[C@@H]2CN ; O=C(O)COCCOCCNC(=O)OCc1ccccc1 ; CC1(C)O[C@@H]2[C@H](O1)[C@@H](Nc1cncc(C(F)(F)F)n1)CO[C@@H]2CNC(=O)COCCOCCNC(=O)OCc1ccccc1 ; CC1(C)O[C@@H]2[C@H](O1)[C@@H](Nc1cncc(C(F)(F)F)n1)CO[C@@H]2CNC(=O)COCCOCCN ; NCCOCCOCC(=O)NC[C@H]1OC[C@H](Nc2cncc(C(F)(F)F)n2)[C@@H](O)[C@H]1O ; "A57"]

Step 1: To a mixture of 3-oxo-1-phenyl-2,7,10-trioxa-4-azadodecan-12-oic acid (282 mg, 0.95 mmol) and HATU (144 mg, 1.05 mmol) in DMF (5 mL) was added DIEA (90 mg, 2.85 mmol) at an ambient temperature, and the reaction mixture was stirred at an ambient temperature for 30 minutes, before the addition of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (300 mg, 0.95 mmol), and the reaction mixture was stirred overnight. The reaction was quenched with H₂O (10 mL) and extracted with ethyl acetate (3×30 mL). The combined organic layers were washed with brine (3×20 mL), and dried over anhydrous Na₂SO₄, filtered. The filtrate was concentrated to give benzyl (2-(2-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethoxy)ethoxy)ethyl)carbamate (460 mg) as crude solid which used in next step directly. LC-MS (ESI) found: 628 [M+H]⁺.
Step 2: To a mixture of benzyl (2-(2-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethoxy)ethoxy)ethyl)carbamate (460 mg, crude) in MeOH (10 mL) was added Pd/C (50 mg) at an ambient temperature and the reaction mixture was stirred at an ambient temperature under a balloon of H2 for 16 h. The mixture was filtered through a pad of celite, the filtrate was concentrated under reduced pressure to give 2-(2-(2-aminoethoxy)ethoxy)-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)acetamide (320 mg) as crude which was used in the next step directly. LC-MS (ESI) found: 494 [M+H]⁺.
Step 3: To a mixture of 2-(2-(2-aminoethoxy)ethoxy)-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)acetamide (320 mg, crude) in THF (10 mL) was added HCl (2.0 N, aq., 0.50 mL) at an ambient temperature and stirred for 0.5 h. The mixture was concentrated and the residue was purified by reverse flash column (5% acetonitrile/water) to give 2-(2-(2-aminoethoxy)ethoxy)-N-(((2R,3R,4R,5S)-3,4-dihydroxy-5-((6-(trifluoromethyl)pyrazin-2-yl)amino)tetrahydro-2H-pyran-2-yl)methyl)acetamide (280 mg, three steps overall yield: 65%) as white solid. LC-MS (ESI) found: 454 [M+H]⁺. ¹H NMR (400 MHz, CD₃OD) δ 8.11 (s, 1H), 8.00 (s, 1H), 4.32 (td, J=10.6, 5.1 Hz, 1H), 4.12 (dd, J=11.0, 5.2 Hz, 1H), 4.00 (dd, J=11.0, 5.1 Hz, 2H), 3.85 (t, J=5.2 Hz, 1H), 3.75-3.60 (m, 6H), 3.60-3.47 (m, 3H), 3.40 (dd, J=13.3, 7.3 Hz, 1H), 3.09 (td, J=10.9, 4.7 Hz, 1H), 2.84 (t, J=5.3 Hz, 2H).

### Preparation of (2R,3R,4R,5S)-2-((pyrazin-2-yloxy)methyl)-5-((6-(trifluoromethyl)pyrazin-2-yl)amino)tetrahydro-2H-pyran-3,4-diol (A60)

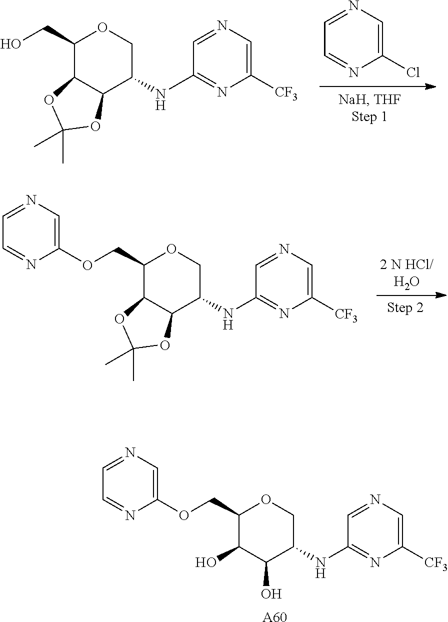

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methanol (100 mg, 0.29 mmol) in THF (10 mL) was added 2-chloropyrazine (49 mg, 0.43 mmol) and NaH (23 mg, 0.58 mmol) under N₂ at an ambient temperature. The mixture was stirred at an ambient temperature overnight. The mixture was diluted with ethyl acetate (10 mL) and washed with water (5 mL×2). The organic layer was dried over sodium sulfate, filtered, and concentrated under reduced pressure to give a crude product. The residue was purified by flash chromatography to give N-((3aR,4R,7S,7aR)-2,2-dimethyl-4-((pyrazin-2-yloxy)methyl)tetrahydro-4H-[1,3]dioxolo[4,5-c]pyran-7-yl)-6-(trifluoromethyl)pyrazin-2-amine (80 mg, 65%) as white solid. LC-MS (ESI) found: 428 [M+H]⁺.
Step 2: To a solution of N-((3aR,4R,7S,7aR)-2,2-dimethyl-4-((pyrazin-2-yloxy)methyl)tetrahydro-4H-[1,3]dioxolo[4,5-c]pyran-7-yl)-6-(trifluoromethyl)pyrazin-2-amine (70 mg, 0.17 mmol) in THF (3 mL) was added HCl/H₂O (0.5 mL, 2 N) at an ambient temperature. The mixture was stirred at an ambient temperature overnight. The pH of the reaction mixture was adjusted to 7 with NH₃·H₂O. The mixture was concentrated, and the residue was purified by revered phase chromatography (MeOH in water from 5% to 95%) to give (2R,3R,4R,5S)-2-((pyrazin-2-yloxy)methyl)-5-((6-(trifluoromethyl)pyrazin-2-yl)amino)tetrahydro-2H-pyran-3,4-diol (52.3 mg, 83%) as white solid. LC-MS (ESI) found: 388 [M+H]⁺. ¹H NMR (400 MHz, CD₃OD): δ 8.23 (d, J=1.3 Hz, 1H), 8.17 (dd, J=2.8, 1.4 Hz, 1H), 8.12 (d, J=3.0 Hz, 2H), 8.00 (s, 1H), 4.55-4.52 (m, 2H), 4.42-4.33 (m, 1H), 4.16 (dd, J=11.0, 5.2 Hz, 1H), 4.02 (d, J=2.6 Hz, 1H), 3.87 (t, J=6.1 Hz, 1H), 3.70 (dd, J=10.6, 3.2 Hz, 1H), 3.17 (t, J=10.8 Hz, 1H).

### Preparation of (2R,3R,4R,5S)-2-((pyrimidin-2-yloxy)methyl)-5-((6-(trifluoromethyl)pyrazin-2-yl)amino)tetrahydro-2H-pyran-3,4-diol (A61)

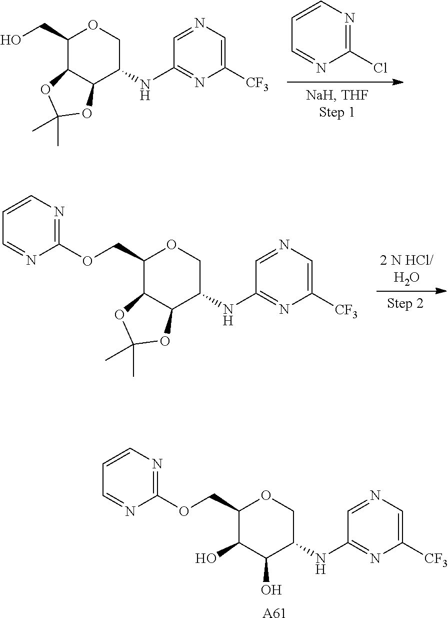

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methanol (100 mg, 0.29 mmol) in THF (10 mL) was added 2-chloropyrimidine (49 mg, 0.43 mmol) and NaH (23 mg, 0.58 mmol) under N₂ at an ambient temperature. The mixture was stirred at an ambient temperature overnight. The mixture was diluted with ethyl acetate (10 mL) and washed with water (5 mL×2). The organic layer was dried over sodium sulfate, filtered, and concentrated under reduced pressure. The residue was purified by flash chromatography to give N-((3aR,4R,7S,7aR)-2,2-dimethyl-4-((pyrimidin-2-yloxy)methyl)tetrahydro-4H-[1,3]dioxolo[4,5-c]pyran-7-yl)-6-(trifluoromethyl)pyrazin-2-amine (100 mg, 81%) as white solid. LC-MS (ESI) found: 428 [M+H]⁺.
Step 2: To a solution of N-((3aR,4R,7S,7aR)-2,2-dimethyl-4-((pyrimidin-2-yloxy)methyl)tetrahydro-4H-[1,3]dioxolo[4,5-c]pyran-7-yl)-6-(trifluoromethyl)pyrazin-2-amine (90 mg, 0.21 mmol) in THF (3 mL) was added HCl (0.5 mL, 2 N) at an ambient temperature. The mixture was stirred at an ambient temperature overnight. The pH of the reaction mixture was adjusted to 7 with NH₃·H₂O. The reaction mixture was purified by Prep-HPLC (MeCN in water from 5% to 70%) to give (2R,3R,4R,5S)-2-((pyrimidin-2-yloxy)methyl)-5-((6-(trifluoromethyl)pyrazin-2-yl)amino)tetrahydro-2H-pyran-3,4-diol (45.6 mg, 56%) as white solid. LC-MS (ESI) found: 388 [M+H]⁺. ¹H NMR (400 MHz, CD₃OD): δ 8.59 (d, J=4.9 Hz, 2H), 8.12 (s, 1H), 8.00 (s, 1H), 7.13 (t, J=4.9 Hz, 1H), 4.57 (qd, J=11.2, 6.1 Hz, 2H), 4.38 (td, J=10.6, 5.1 Hz, 1H), 4.15 (dd, J=11.0, 5.2 Hz, 1H), 4.04 (d, J=2.5 Hz, 1H), 3.87 (dd, J=6.7, 5.4 Hz, 1H), 3.71 (dd, J=10.6, 3.2 Hz, 1H), 3.17 (t, J=10.8 Hz, 1H).

### Preparation of 2-(4-((((2R,3R,4R,5S)-3,4-dihydroxy-5-((6-(trifluoromethyl)pyrazin-2-yl)amino)tetrahydro-2H-pyran-2-yl)methyl)carbamoyl)-[1,4′-bipiperidin]-1′-yl)acetic acid (A62)

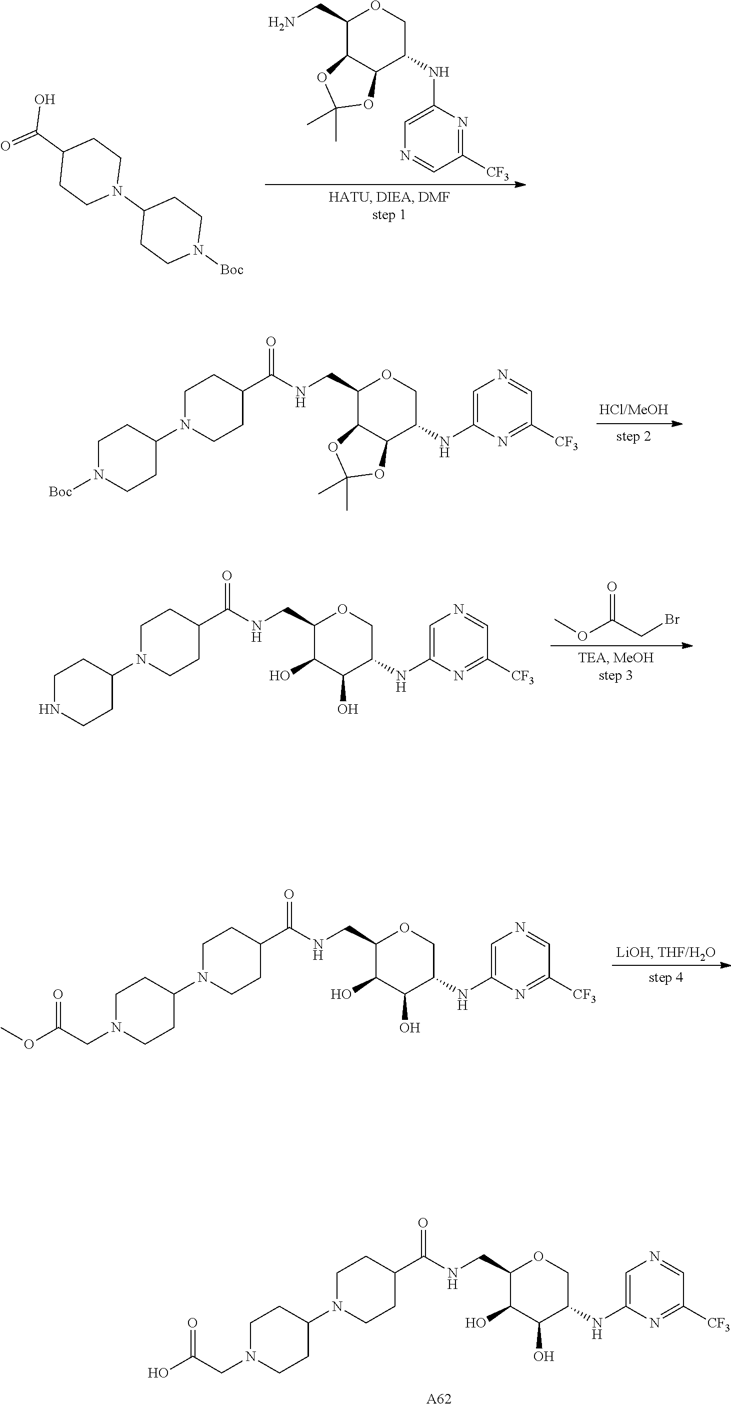

Step 1: To a mixture of 1′-(tert-butoxycarbonyl)-[1,4′-bipiperidine]-4-carboxylic acid (450 mg, 1.44 mmol) and HATU (655 mg, 1.58 mmol) in DMF (5 mL) was added DIEA (375 mg, 2.88 mmol) at an ambient temperature, and the reaction mixture was stirred at an ambient temperature for 0.5 h, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (500 mg, 1.44 mmol) was added into the solution and the mixture was stirred at an ambient temperature overnight. The reaction was quenched with H₂O (10 mL) and the mixture was extracted with ethyl acetate (3×30 mL). The combined organic layers were washed with brine (3×20 mL), and dried over anhydrous Na₂SO₄, filtered. The filtrate was concentrated and the residue was purified by silica gel chromatography (DCM/MeOH=20:1) to give tert-butyl 4-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-[1,4′-bipiperidine]-1′-carboxylate (577 mg, 62.6% yield) as yellow solid. LC-MS (ESI) found: 643.2 [M+H]⁺.
Step 2: To a mixture of tert-butyl 4-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-[1,4′-bipiperidine]-1′-carboxylate (577 mg, 0.89 mmol) in DCM (10 mL) was added TFA (2 mL), and the reaction mixture was stirred at an ambient temperature for 2 h. After completion, the mixture was concentrated under reduced pressure to give N-(((2R,3R,4R,5S)-3,4-dihydroxy-5-((6-(trifluoromethyl)pyrazin-2-yl)amino)tetrahydro-2H-pyran-2-yl)methyl)-[1,4′-bipiperidine]-4-carboxamide as crude which used in next step directly. LC-MS (ESI) found: 503.2 [M+H]⁺.
Step 3: To a mixture of N-(((2R,3R,4R,5S)-3,4-dihydroxy-5-((6-(trifluoromethyl)pyrazin-2-yl)amino)tetrahydro-2H-pyran-2-yl)methyl)-[1,4′-bipiperidine]-4-carboxamide (500 mg crude) in MeOH (10 mL) was added methyl 2-bromoacetate (138 mg, 0.91 mmol) and TEA (0.6 mL) at an ambient temperature, and the reaction mixture was stirred at an ambient temperature for 1 h. After completion, the mixture was concentrated under reduced pressure to give methyl 2-(4-((((2R,3R,4R,5S)-3,4-dihydroxy-5-((6-(trifluoromethyl)pyrazin-2-yl)amino)tetrahydro-2H-pyran-2-yl)methyl)carbamoyl)-[1,4′-bipiperidin]-1′-yl)acetate as crude which used in next step directly. LC-MS (ESI) found: 575.2 [M+H]⁺.
Step 4: To a mixture of methyl 2-(4-((((2R,3R,4R,5S)-3,4-dihydroxy-5-((6-(trifluoromethyl)pyrazin-2-yl)amino)tetrahydro-2H-pyran-2-yl)methyl)carbamoyl)-[1,4′-bipiperidin]-1′-yl)acetate (400 mg crude) in THF/H₂O (3:1, 6 mL) was added LiOH (200 mg, 3.9 mmol), and the reaction mixture was stirred at an ambient temperature for 1 h. After completion, the mixture was concentrated under reduced pressure and the residue was purified by reversed phase flash column (20% MeOH in pure water) to give 2-(4-((((2R,3R,4R,5S)-3,4-dihydroxy-5-((6-(trifluoromethyl)pyrazin-2-yl)amino)tetrahydro-2H-pyran-2-yl)methyl)carbamoyl)-[1,4′-bipiperidin]-1′-yl)acetic acid (300 mg, 59.5% yield) as gray solid. LC-MS (ESI) found: 561.2 [M+H]⁺. ¹H NMR (400 MHz, CD₃OD) δ 8.20 (s, 1H), 8.11 (s, 1H), 8.00 (s, 1H), 4.31 (td, J=10.6, 5.2 Hz, 1H), 4.12 (dd, J=11.0, 5.2 Hz, 1H), 3.83 (d, J=3.2 Hz, 1H), 3.68-3.55 (m, 3H), 3.54-3.47 (m, 4H), 3.38-3.32 (m, 3H), 3.14-2.97 (m, 2H), 2.89 (t, J=11.9 Hz, 2H), 2.74 (t, J=10.5 Hz, 2H), 2.48-2.37 (m, 1H), 2.17 (d, J=12.9 Hz, 2H), 2.03-1.80 (m, 6H).

### Preparation of N-(((2R,3R,4R,5S)-3,4-dihydroxy-5-((6-(trifluoromethyl)pyrazin-2-yl)amino)tetrahydro-2H-pyran-2-yl)methyl)nicotinamide (A63)

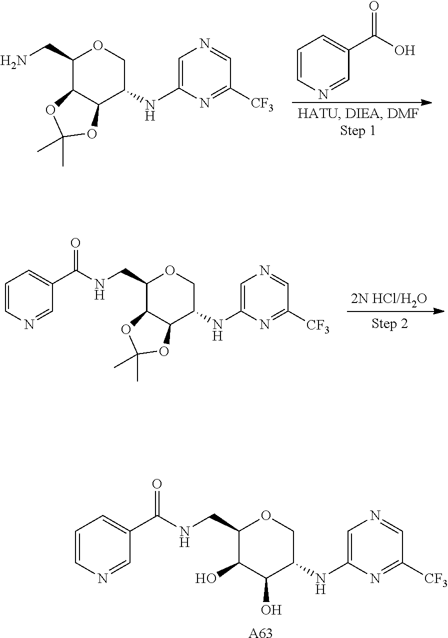

Step 1: To a solution of nicotinic acid (20 mg, 0.16 mmol) in DMF (3 mL) were added HATU (65 mg, 0.17 mmol) and DIEA (37 mg, 0.29 mmol) at an ambient temperature. After stirring at an ambient temperature for 20 min, N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added. The reaction mixture was stirred at an ambient temperature overnight, quenched with water (10 mL) and extracted with ethyl acetate (30 mL). The organic layer is concentrated and the residue was purified by flash column to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)nicotinamide (50 mg, 77%) as brown oil. LC-MS (ESI) found: 454 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)nicotinamide (50 mg, 0.11 mmol) in THF (3 mL) was added HCl/H₂O (0.5 mL, 2 N) at an ambient temperature. The reaction mixture was stirred at an ambient temperature overnight. The pH of the mixture was adjusted to 7 with NH₃·H₂O, concentrated, and the residue was purified by Prep-HPLC (MeCN in water from 5% to 70%) to give N-(((2R,3R,4R,5S)-3,4-dihydroxy-5-((6-(trifluoromethyl)pyrazin-2-yl)amino)tetrahydro-2H-pyran-2-yl)methyl)nicotinamide (20 mg, 44%) as white solid. LC-MS (ESI) found: 403 [M+H]⁺. ¹H NMR (400 MHz, CD₃OD) δ 8.98 (dd, J=2.2, 0.7 Hz, 1H), 8.68 (dd, J=4.9, 1.5 Hz, 1H), 8.28-8.22 (m, 1H), 8.11 (s, 1H), 8.00 (s, 1H), 7.54 (dd, J=8.0, 4.9 Hz, 1H) 4.35 (td, J=10.6, 5.1 Hz, 1H), 4.15 (dd, J=11.0, 5.2 Hz, 1H), 3.92 (d, J=3.0 Hz, 1H), 3.74 (dd, J=13.3, 4.5 Hz, 1H), 3.68 (dd, J=9.0, 3.8 Hz, 2H), 3.55 (dd, J=13.2, 7.6 Hz, 1H), 3.12 (t, J=10.9 Hz, 1H).

### Preparation of 3,3′-((2-(6-azidohexanamido)propane-1,3-diyl)bis(oxy))bis(N-(1-((2R,3R,4R,5S)-3,4-dihydroxy-5-((6-(trifluoromethyl)pyrazin-2-yl)amino)tetrahydro-2H-pyran-2-yl)-2,5,8,11-tetraoxatridecan-13-yl)propanamide) (A64)

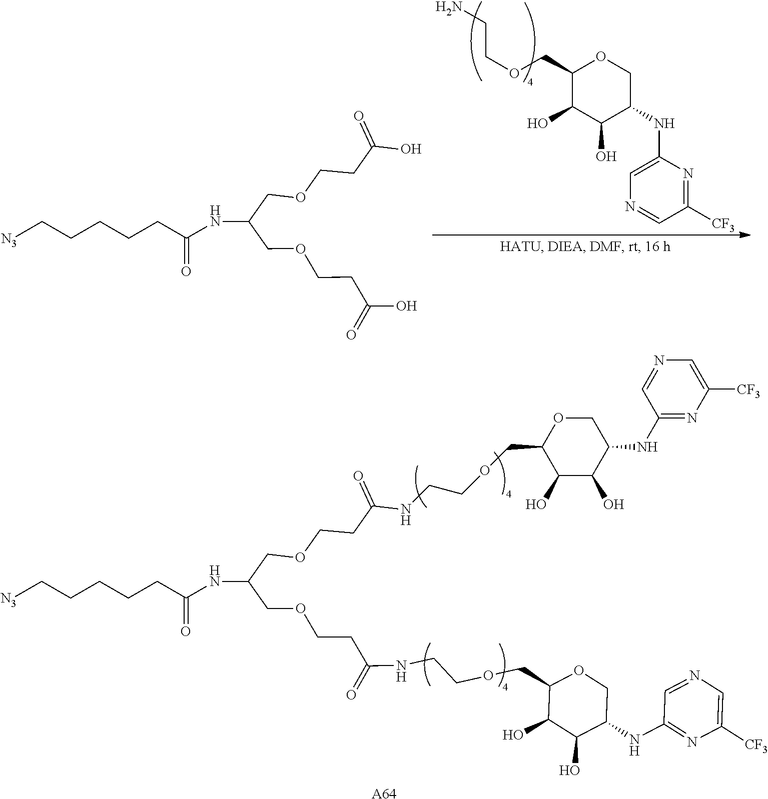

To a mixture of 3,3′-((2-(6-azidohexanamido)propane-1,3-diyl)bis(oxy))dipropionic acid (168 mg, 0.45 mmol) and HATU (513 mg, 1.35 mmol) in DMF (6 mL) was added DIEA (232 mg, 1.80 mmol) at an ambient temperature, and the reaction mixture was stirred at an ambient temperature for 0.5 h, then (2R,3R,4R,5S)-2-(13-amino-2,5,8,11-tetraoxatridecyl)-5-((6-(trifluoromethyl)pyrazin-2-yl)amino)tetrahydro-2H-pyran-3,4-diol (500 mg, 1.03 mmol) was added into the solution and stirred overnight. After completion, the mixture was purified by reversed flash column (5% acetonitrile) to give 3,3′-((2-(6-azidohexanamido)propane-1,3-diyl)bis(oxy))bis(N-(1-((2R,3R,4R,5S)-3,4-dihydroxy-5-((6-(trifluoromethyl)pyrazin-2-yl)amino)tetrahydro-2H-pyran-2-yl)-2,5,8,11-tetraoxatridecan-13-yl)propanamide) (300 mg, 51% yield) as yellow oil. LC-MS (ESI) found: 1308.3 [M+H]⁺. ¹H NMR (400 MHz, CD₃OD) δ 8.19 (s, 1H), 8.00 (s, 1H), 4.56 (d, J=8.3 Hz, 1H), 4.23-4.03 (m, 2H), 4.03-3.85 (m, 2H), 3.84-3.63 (m, 7H), 3.61-3.40 (m, 15H), 3.40-3.32 (m, 3H), 2.43 (t, J=6.1 Hz, 2H), 2.22 (t, J=7.4 Hz, 1H), 1.70-1.52 (m, 2H), 1.48-1.34 (m, 1H).

### Preparation of (1R,2S,3R,6S)-3-(hydroxymethyl)-6-((4-(trifluoromethyl)pyrimidin-2-yl)amino)cyclohexane-1,2-diol (A65)

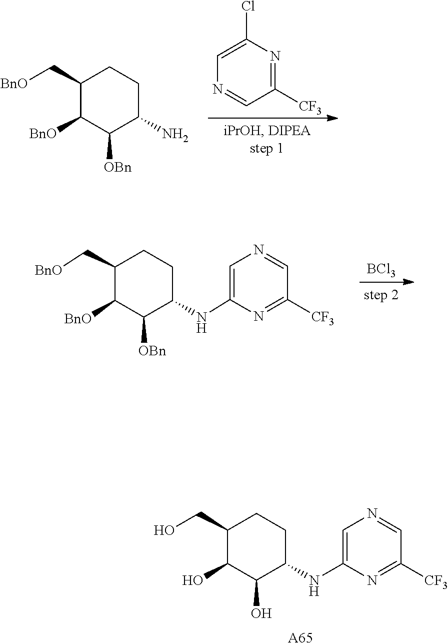

Step 1: A solution of (1S,2R,3S,4R)-2,3-bis(benzyloxy)-4-((benzyloxy)methyl)cyclohexan-1-amine (40 mg, 0.09 mmol), 2-chloro-6-(trifluoromethyl)pyrazine (20 mg, 0.11 mmol) and DIPEA (24 mg, 0.19 mmol) in DMF (2 mL) was stirred at 100° C. under N₂ overnight. The mixture was cooled to rt, concentrated, and the residue was purified by flash column to give N-((1S,2R,3S,4R) 2,3-bis(benzyloxy)-4-((benzyloxy)methyl)cyclohexyl)-6-(trifluoromethyl)pyrazin-2-amine (15 mg, 58%) as colorless oil. LC-MS (ESI) found: 432 [M+H]⁺.
Step 2: To a solution of N-((1S,2R,3S,4R)-2,3-bis(benzyloxy)-4-((benzyloxy)methyl)cyclohexyl)-6-(trifluoromethyl)pyrazin-2-amine (15 mg, 0.03 mmol) in DCM (2 mL) was added BCl₃ (0.5 mL) at 0° C. The reaction mixture was stirred at an ambient temperature overnight, then quenched with NH₄₀H. The residue was purified by flash column to give (1R,2S,3R,6S)-3-(hydroxymethyl)-6-((6-(trifluoromethyl)pyrazin-2-yl)amino)cyclohexane-1,2-diol (2.9 mg, 37%) as white solid. LC-MS (ESI) found: 308[M+H]⁺. ¹H NMR (400 MHz, CD₃OD) δ 8.07 (s, 1H), 7.93 (s, 1H), 4.18 (d, J=4.2 Hz, 1H), 4.08 (d, J=2.4 Hz, 1H), 3.66 (dd, J=10.6, 7.3 Hz, 1H), 3.49 (ddd, J=13.1, 10.5, 4.5 Hz, 2H), 2.09 (dd, J=12.8, 4.3 Hz, 1H), 1.66-1.59 (m, 1H), 1.58-1.43 (m, 2H), 1.37-1.14 (m, 1H).

### Preparation of 4³-((((2R,3R,4R,5S)-3,4-dihydroxy-5-((6-(trifluoromethyl)pyrazin-2-yl)amino)tetrahydro-2H-pyran-2-yl)methyl)carbamoyl)-1,4(1,4)-dibenzenacyclohexaphane-1²-carboxylic acid (A66)

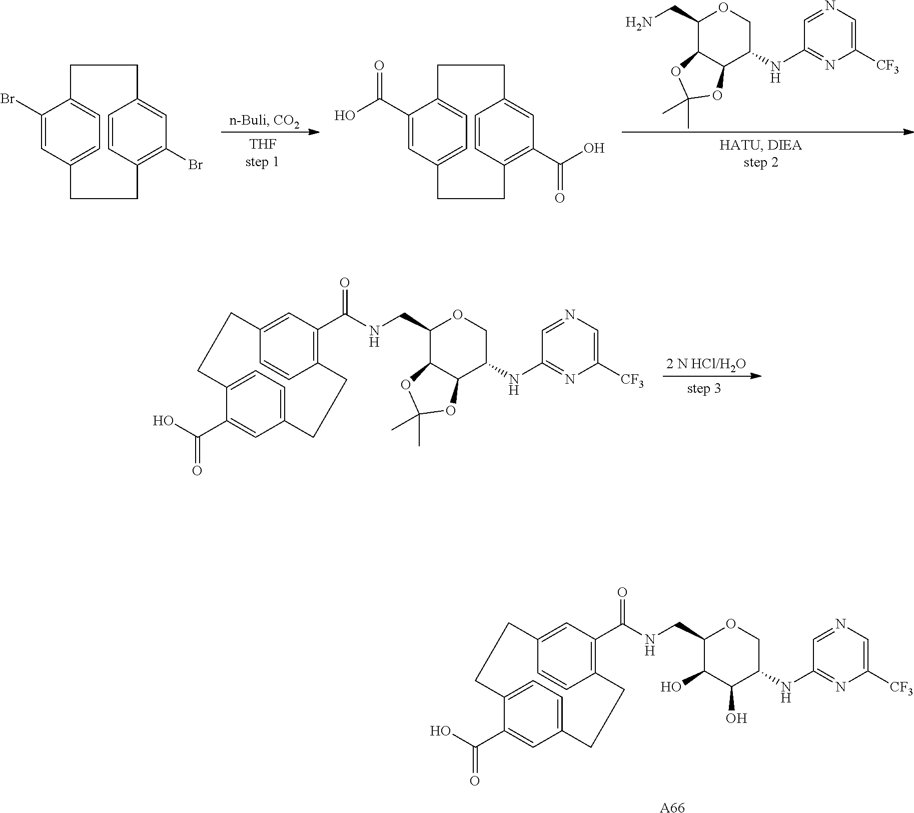

Step 1: To a solution of 1²,4³-dibromo-1,4(1,4)-dibenzenacyclohexaphane (1.0 g, 2.75 mmol) in THF (50.0 mL) at −30° C. was added n-BuLi (5.5 mL, 5.50 mmol, 1.0 N). The mixture was stirred at −30° C. for 1 h and then stirred at the room temperature under a balloon of CO2 for 3 h. The reaction mixture was quenched with MeOH (15.0 mL) and diluted with ethyl acetate (200 mL) and washed with water (20 mL×3). The organic layer was dried over sodium sulfate, filtered, concentrated under reduced pressure to give a crude product, which was purified by column chromatography to give 1,4(1,4)-dibenzenacyclohexaphane-12,43-dicarboxylic acid (650.0 mg, 2.20 mmol, 80%). LC-MS (ESI) found: 297 [M+H]⁺.
Step 2: To a solution of 1,4(1,4)-dibenzenacyclohexaphane-1²,4³-dicarboxylic acid (650 mg, 2.20 mmol) in DMF (20.0 mL) was added N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (766 mg, 2.20 mmol), HATU (836 mg, 2.20 mmol), DIEA (864 mg, 6.6 mmol). The reaction mixture was stirred at the room temperature for 16 h. The mixture was diluted with ethyl acetate (100 mL) and washed with water (10 mL×3), the organic layer was dried over sodium sulfate, filtered, and concentrated under reduced pressure to give a crude product, which was purified by flash column (PE/EA=1/1) to give 4³-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-1,4(1,4)-dibenzenacyclohexaphane-12-carboxylic acid (939.0 mg, 1.5 mmol, 68.2%). LC-MS (ESI) found: 627 [M+H]⁺.
Step 3: To a solution of 4³-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-1,4(1,4)-dibenzenacyclohexaphane-1²-carboxylic acid (470 mg, 0.75 mmol) in THF (20.0 mL) was added HCl/H₂O (20.0 mL, 3.0 N), the mixture was stirred at the room temperature for 16 h. The mixture was concentrated under reduced pressure, the residue was purified in prep-HPLC to give 4³-((((2R,3R,4R,5S)-3,4-dihydroxy-5-((6-(trifluoromethyl)pyrazin-2-yl)amino)tetrahydro-2H-pyran-2-yl)methyl)carbamoyl)-1,4(1,4)-dibenzenacyclohexaphane-12-carboxylic acid (50.0 mg, 0.085 mmol, 11.3%). LC-MS (ESI) found: 587 [M+H]⁺. ¹H NMR (400 MHz, CD₃OD) δ 8.12 (s, 1H), 8.00 (d, J=3.9 Hz, 1H), 6.88 (ddd, J=15.8, 7.8, 1.9 Hz, 1H), 6.79 (dd, J=4.6, 1.8 Hz, 1H) 6.65 (d, J=7.8 Hz, 1H), 6.53 (ddd, J=12.1, 9.6, 6.1 Hz, 2H), 4.44-4.31 (m, 1H), 4.23-4.03 (m, 2H), 3.95 (d, J=3.1 Hz, 1H), 3.78-3.61 (m, 4H), 3.57-3.41 (m, 1H), 3.23-3.04 (m, 5H) 3.04-2.86 (m, 2H). 19F NMR (377 MHz, CD₃OD) δ −70.31 (d, J=4.3 Hz).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5-phenylpicolinamide (A67)

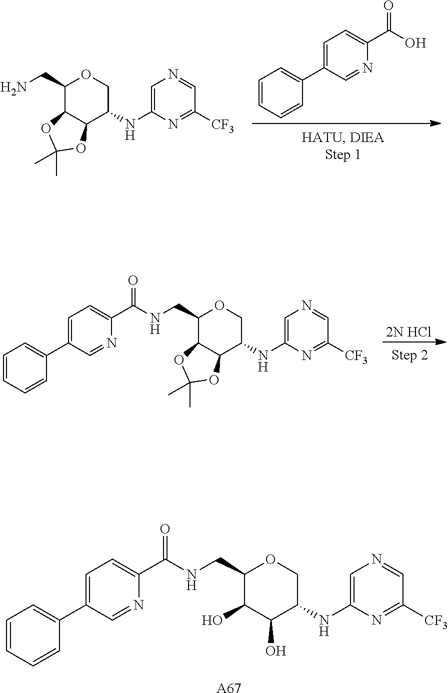

Step 1: To a solution of 5-phenylpicolinic acid (34 mg, 0.24 mmol) in DMF (3 mL) were added HATU (98 mg, 0.27 mmol) and DIEA (37 mg, 0.29 mmol) at an ambient temperature. The mixture was stirred at an ambient temperature for 20 min, before addition of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol). The mixture was stirred at an ambient temperature overnight, quenched with water (10 mL), and extracted with ethyl acetate (30 mL×2). The combined organic layer was dried over sodium sulfate, and concentrated. The residue was purified by flash chromatography to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-5-phenylpicolinamide (50 mg, 66%) as brown oil. LC-MS (ESI) found: 530 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-5-phenylpicolinamide (50 mg, 0.10 mmol) in THF (3 mL) was added HCl/H₂O (0.5 mL, 2 N). The mixture was stirred at an ambient temperature overnight. The pH of the reaction mixture was adjusted to 7 with NH₃·H₂O. The reaction mixture was concentrated, and the residue was purified by Prep-HPLC (MeCN in water from 5% to 70%.) to give N-(((2R,3R,4R,5 S)-3,4-dihydroxy-5-((6-(trifluoromethyl)pyrazin-2-yl)amino)tetrahydro-2H-pyran-2-yl)methyl)-5-phenylpicolinamide (20 mg, 45%) as white solid. LC-MS (ESI) found: 403 [M+H]⁺. ¹H NMR (400 MHz, CD₃OD): δ 8.89 (d, J=1.3 Hz, 1H), 8.22-8.14 (m, 2H), 8.11 (s, 1H), 8.00 (s, 1H), 7.74-7.69 (m, 2H), 7.52 (t, J=7.4 Hz, 2H), 7.48-7.42 (m, 1H), 4.41-4.33 (m, 1H), 4.16 (dd, J=11.0, 5.2 Hz, 1H), 3.93 (d, J=3.2 Hz, 1H), 3.78 (dd, J=12.5, 4.0 Hz, 1H), 3.65 (ddd, J=20.3, 11.6, 5.5 Hz, 3H), 3.15 (t, J=10.8 Hz, 1H).

### Preparation of (2R,3R,4R,5S)-2-((1,4-diazepan-1-yl)methyl)-5-((6-(trifluoromethyl)pyrazin-2-yl)amino)tetrahydro-2H-pyran-3,4-diol (A68)

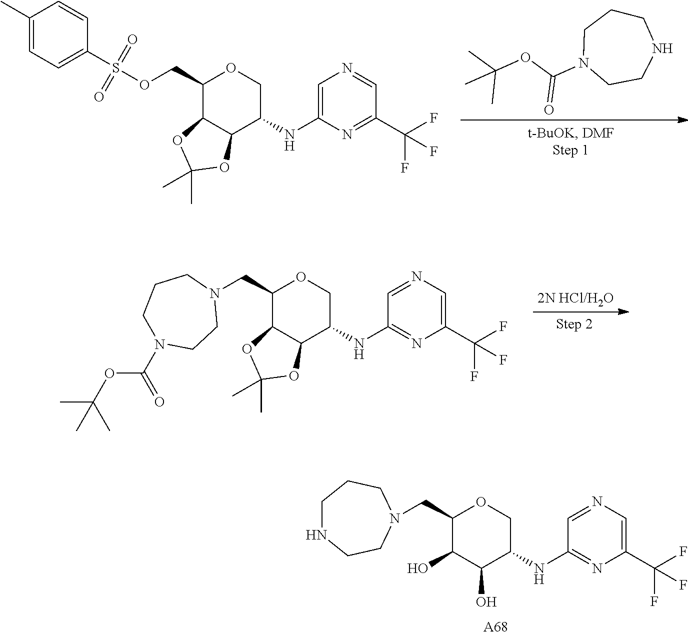

Step 1: A solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (150 mg, 0.3 mmol), tert-butyl 1,4-diazepane-1-carboxylate (179 mg, 0.9 mmol) and TEA (90 mg, 0.9 mmol) in DMF (2 mL) was stirred at 100° C. overnight. The reaction mixture was cooled to rt and quenched with water (10 mL), extracted with ethyl acetate (20 mL×3), the combined organic layer was concentrated. The residue was purified by flash chromatography with reversed phase (C18, MeOH in H₂O from 5% to 95%) to give tert-butyl 4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1,4-diazepane-1-carboxylate (150 mg, 94% yield) as a yellow oil. LC-MS (ESI) found: 532 [M+H]⁺.
Step 2: A solution of tert-butyl 4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1,4-diazepane-1-carboxylate (150 mg, 0.29 mmol) in HCl (2.0 mL, 3.0 N) and THE (1.0 mL) was stirred at an ambient temperature for 3 h. The reaction was quenched by NH₄₀H (0.5 mL) and the mixture was concentrated. The residue was purified by Prep-HPLC to give (2R,3R,4R,5S)-2-((1,4-diazepan-1-yl)methyl)-5-((6-(trifluoromethyl)pyrazin-2-yl)amino)tetrahydro-2H-pyran-3,4-diol (38 mg, 34% yield) as a white solid. LC-MS (ESI) found: 392 [M+H]⁺. ¹H NMR (400 MHz, CD₃OD) δ 8.14 (s, 1H), 8.01 (s, 1H), 4.34 (td, J=10.6, 5.1 Hz, 1H), 4.17 (dd, J=11.0, 5.1 Hz, 1H), 3.92-3.87 (m, 1H), 3.84-3.78 (m, 1H), 3.74-3.68 (m, 1H), 3.51-3.40 (m, 4H), 3.38-3.33 (m, 2H), 3.28-3.21 (m, 3H), 3.20-3.07 (m, 2H), 2.15 (s, 2H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-1H-imidazo[4,5-b]pyridine-5-carboxamide (A69)

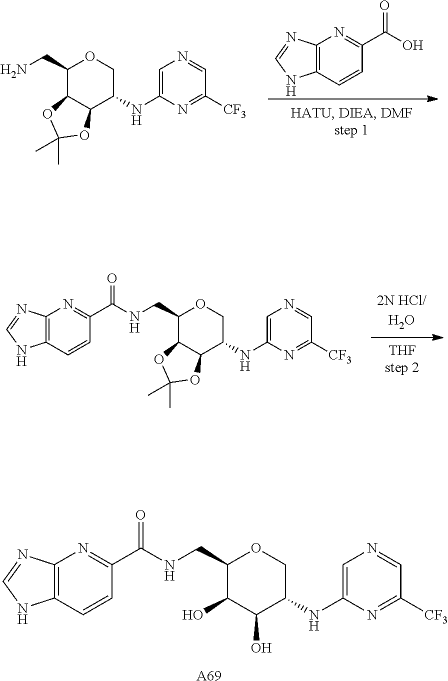 [SMILES: CC1(C)O[C@@H]2[C@H](O1)[C@@H](Nc1cncc(C(F)(F)F)n1)CO[C@@H]2CN ; O=C(O)c1ccc2[nH]cnc2n1 ; CC1(C)O[C@@H]2[C@H](O1)[C@@H](Nc1cncc(C(F)(F)F)n1)CO[C@@H]2CNC(=O)c1ccc2[nH]cnc2n1 ; O=C(NC[C@H]1OC[C@H](Nc2cncc(C(F)(F)F)n2)[C@@H](O)[C@H]1O)c1ccc2[nH]cnc2n1 ; "A69"]

Step 1: A solution of 1H-imidazo[4,5-b]pyridine-5-carboxylic acid (28 mg, 0.17 mmol), HATU (66 mg, 0.17 mmol) and DIEA (0.07 mL, 0.43 mmol) in DMF (2.0 mL) was stirred at an ambient temperature for 30 min. N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) was added at an ambient temperature. The reaction mixture was stirred at an ambient temperature for 16 hours. The resulting mixture was diluted with ethyl acetate (50 mL), washed with H₂O (20 mL) and brine (10 mL), dried over Na₂SO₄. The organic layer was concentrated in vacuo, and the residue was purified by flash chromatography (silica gel, 0˜10% MeOH in DCM) to give N-(((3 aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-imidazo[4,5-b]pyridine-5-carboxamide (25 mg, yield: 35%) as a colorless oil. LC-MS (ESI) found: 494 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1H-imidazo[4,5-b]pyridine-5-carboxamide (25 mg, 0.05 mmol) in THF (3.0 mL) was added HCl (1.0 mL, 2.0 N). The reaction was stirred at an ambient temperature for 3 hrs. The resulting mixture was neutralized with ammonia, extracted with EtOAc (30 mL×2), the combined organic layers was concentrated in vacuo. The crude product was purified by Prep-HPLC to give N-(((2R,3R,4R,5S)-3,4-dihydroxy-5-((6-(trifluoromethyl)pyrazin-2-yl)amino)tetrahydro-2H-pyran-2-yl)methyl)-1H-imidazo[4,5-b]pyridine-5-carboxamide (8.4 mg, yield: 36%) as a white solid. LC-MS (ESI) found: 454 [M+H]⁺. ¹H NMR (400 MHz, CD₃OD): δ 8.60 (s, 1H), 8.14 (t, J=5.1 Hz, 2H), 8.11 (s, 1H), 8.00 (s, 1H) 4.37 (td, J=10.6, 5.1 Hz, 1H), 4.17 (dd, J=11.0, 5.2 Hz, 1H), 3.94 (d, J=3.1 Hz, 1H), 3.80 (dd, J=12.7, 4.1 Hz, 1H), 3.65 (ddd, J=20.4, 10.0, 6.8 Hz, 3H), 3.15 (t, J=10.8 Hz, 1H). ¹⁹F NMR (377 MHz, CD₃OD): δ −70.31 (s).

### Preparation of (2R,3R,4R,5S)-2-(azepan-1-ylmethyl)-5-((6-(trifluoromethyl)pyrazin-2-yl)amino)tetrahydro-2H-pyran-3,4-diol (A70)

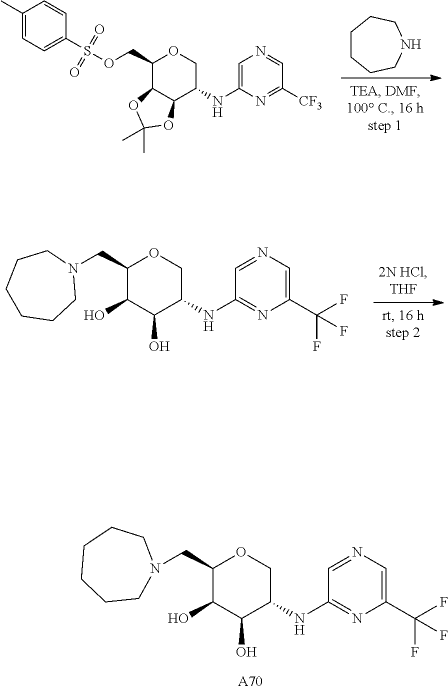

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (100 mg, 0.19 mmol) in DMF (5 mL) was added azepane (20 mg, 0.19 mmol) and TEA (2 mL), the reaction mixture was stirred at 100° C. for 16 hrs. The reaction mixture was cooled to rt, quenched with 10 mL water and extracted with ethyl acetate (10 mL×3), the combined organic layers was dried over anhydrous Na₂SO₄ and concentrated under vacuum to give crude N-((3aS,4R,7S,7aR) 4-(azepan-1-ylmethyl)-2,2-dimethyltetrahydro-4H-[1,3]dioxolo[4,5-c]pyran-7-yl)-6-(trifluoromethyl)pyrazin-2-amine as yellow gel which used in next step directly. LC-MS (ESI) found: 431 [M+H]⁺.
Step 2: To a solution of N-((3aS,4R,7S,7aR)-4-(azepan-1-ylmethyl)-2,2-dimethyltetrahydro-4H-[1,3]dioxolo[4,5-c]pyran-7-yl)-6-(trifluoromethyl)pyrazin-2-amine (crude) in THF (5 mL) was added 2.0 N HCl (2.0 mL), then the mixture was stirred at an ambient temperature for 16 h. The reaction mixture was added NH₄₀H until pH=7, and was extracted with ethyl acetate (30 mL×2), the combined organic layers was concentrated, the residue was purified by reversed phase flash column chromatography (30% MeCN in water) to give (2R,3R,4R,5S)-2-(azepan-1-ylmethyl)-5-((6-(trifluoromethyl)pyrazin-2-yl)amino)tetrahydro-2H-pyran-3,4-diol as white solid (5.5 mg, yield: 6.5%). LC-MS (ESI) found: 391 [M+H]⁺. ¹H NMR (400 MHz, CD₃OD): δ 8.11 (s, 1H) 8.00 (s, 1H), 4.33 (td, J=10.6, 5.2 Hz, 1H), 4.12 (dd, J=11.0, 5.2 Hz, 1H), 3.90 (d, J=3.0 Hz, 1H), 3.64 (dd, J=10.5, 3.2 Hz, 1H), 3.58-3.50 (m, 1H), 3.10 (t, J=10.8 Hz, 1H), 2.87 (t, J=5.7 Hz, 2H), 2.83 (dd, J=10.1, 4.4 Hz, 4H), 1.70 (d, J=3.8 Hz, 4H), 1.66-1.60 (m, 4H).

### Preparation of N-(((2R,3R,4R,5S)-3,4-dihydroxy-5-((6-(trifluoromethyl)pyrazin-2-yl)amino)tetrahydro-2H-pyran-2-yl)methyl)quinoxaline-2-carboxamide (A71)

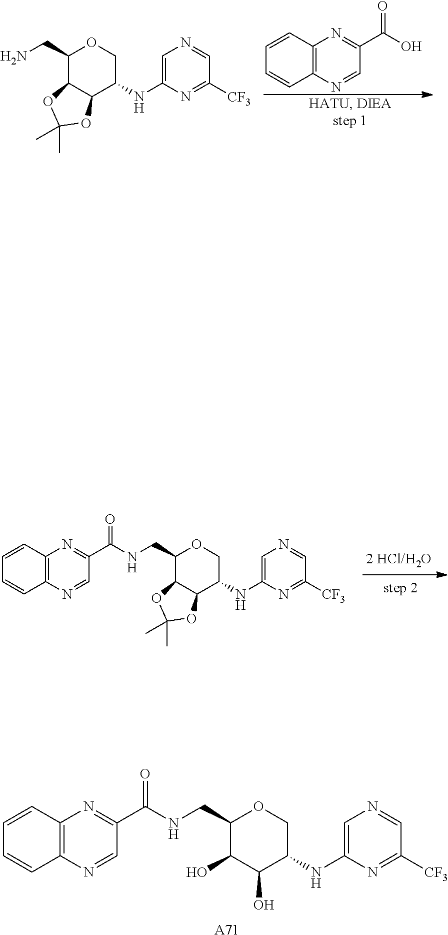 [SMILES: CC1(C)O[C@@H]2[C@H](O1)[C@@H](Nc1cncc(C(F)(F)F)n1)CO[C@@H]2CN ; O=C(O)c1cnc2ccccc2n1 ; CC1(C)O[C@@H]2[C@H](O1)[C@@H](Nc1cncc(C(F)(F)F)n1)CO[C@@H]2CNC(=O)c1cnc2ccccc2n1 ; O=C(NC[C@H]1OC[C@H](Nc2cncc(C(F)(F)F)n2)[C@@H](O)[C@H]1O)c1cnc2ccccc2n1 ; "A71"]

Step 1: To a solution of quinoxaline-2-carboxylic acid (60 mg, 0.17 mmol) in DMF (3 mL) were added HATU (160 mg, 0.42 mmol) and DIEA (66 mg, 0.51 mmol), then the mixture was stirred at 0° C. for 30 minutes. N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (45 mg, 0.25 mmol) in DMF (0.5 ml) was added to the mixture, and the reaction was stirred at an ambient temperature for 1.5 h. The reaction mixture was quenched with water (20 mL), extracted with ethyl acetate (30 mL×2), the combined organic layers was concentrated, the residue was purified by Prep-HPLC to give N-(((3aS,4R,7S,7aR)-2,2-dimethyl-7-((6-(trifluoromethyl) pyrazin-2-yl)amino)tetrahydro-4H-[1,3]dioxolo[4,5-c]pyran-4-yl)methyl)quinoxaline-2-carboxamide as white solid (75 mg, yield: 87%). LC-MS (ESI) found: 505 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinoxaline-2-carboxamide (75 mg, 0.15 mmol) in THF (2.0 ml) was added 2 N HCl (1.0 mL), then the mixture was stirred at an ambient temperature for 1 h. The pH of the reaction mixture was adjusted to 7 with ammonia, and concentrated. The residue was purified by Prep-HPLC (MeCN/H₂O, 0.1% formic acid) to give N-(((2R,3R,4R,5S)-3,4-dihydroxy-5-((6-(trifluoromethyl)pyrazin-2-yl)amino)tetrahydro-2H-pyran-2-yl)methyl)quinoxaline-2-carboxamide as yellow solid (7 mg, yield: 10%). LC-MS (ESI) found: 465 [M+H]⁺. ¹H NMR (400 MHz, CD₃OD): 9.51 (d, J=1.1 Hz, 1H), 8.26-8.20 (m, 1H), 8.17 (d, J=7.1 Hz, 1H), 8.11 (s, 1H), 8.00 (s, 1H), 7.98-7.90 (m, 2H), 4.38 (m, 1H), 4.17 (dd, J=11.0, 5.2 Hz, 1H), 3.97 (d, J=3.1 Hz, 1H), 3.84 (dd, J=12.4, 3.6 Hz, 1H), 3.75-3.62 (m, 3H), 3.15 (t, J=10.9 Hz, 1H).

### Preparation of methyl 2-((((2R,3R,4R,5S)-3,4-dihydroxy-5-((6-(trifluoromethyl)pyrazin-2-yl)amino)tetrahydro-2H-pyran-2-yl)methyl)carbamoyl)pyrimidine-5-carboxylate (A72)

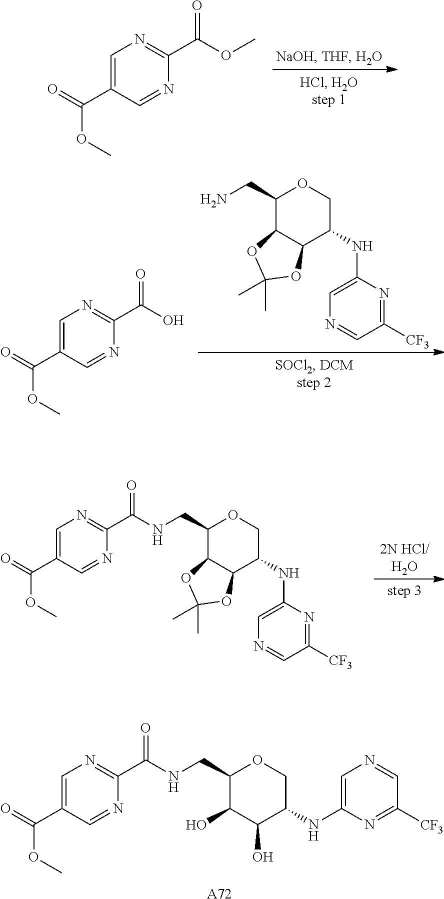 [SMILES: COC(=O)c1cnc(C(=O)OC)nc1 ; COC(=O)c1cnc(C(=O)O)nc1 ; CC1(C)O[C@@H]2[C@H](O1)[C@@H](Nc1cncc(C(F)(F)F)n1)CO[C@@H]2CN ; COC(=O)c1cnc(C(=O)NC[C@H]2OC[C@H](Nc3cncc(C(F)(F)F)n3)[C@H]3OC(C)(C)O[C@H]32)nc1 ; COC(=O)c1cnc(C(=O)NC[C@H]2OC[C@H](Nc3cncc(C(F)(F)F)n3)[C@@H](O)[C@H]2O)nc1 ; "A72"]

Step 1: To a solution of dimethyl pyrimidine-2,5-dicarboxylate (500 mg, 2.5 mmol) in MeOH (10 mL) was added NaOH (100 mg, 2.5 mmol). Then the mixture was stirred at 70° C. for 1 h. To the mixture was added 2 N HCl/H₂O (1.3 mL). The mixture was concentrated in vacuo. The crude was washed with DCM (3 mL) to give 5-(methoxycarbonyl)pyrimidine-2-carboxylic acid as yellow solid which was used to next steps directly. (417 mg, yield: 90%). LC-MS (ESI) found: 183 [M+H]⁺.
Step 2: A solution of 5-(methoxycarbonyl)pyrimidine-2-carboxylic acid (300 mg, 1.64 mmol) in SOCl₂ (5 mL) was stirred at 85° C. under N₂ overnight. The mixture was concentrated. The crude was dissolved in DCM (20 mL). To the mixture were added TEA (3 mL) and N-((3aS,4R,7S,7aR) 4-(aminomethyl)-2,2-dimethyltetrahydro-4H-[1,3]dioxolo[4,5-c]pyran-7-yl)-6-(trifluoromethyl)pyrazin-2-amine (600 mg, 1.72 mmol). The mixture was stirred at an ambient temperature under N₂ overnight. The mixture was concentrated in vacuo. The crude was purified by flash silica column chromatography (eluent=3% MeOH in DCM) to give methyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)pyrimidine-5-carboxylate (350 mg, yield: 41%) as yellow solid. LC-MS (ESI) found: 513 [M+H]⁺.
Step 3: To a solution of methyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)pyrimidine-5-carboxylate (12 mg, 0.02 mmol) in THF (1.5 mL) was added 2 N HCl (0.5 mL). The mixture was stirred at an ambient temperature for 4 h. The pH of the reaction mixture was adjusted to 7 and concentrated. The residue was purified by Prep-HPLC (MeCN/H₂O, 0.1% FA) to give methyl 2-((((2R,3R,4R,5S)-3,4-dihydroxy-5-((6-(trifluoromethyl)pyrazin-2-yl)amino)tetrahydro-2H-pyran-2-yl)methyl)carbamoyl)pyrimidine-5-carboxylate as white solid (2 mg, yield: 18%). LC-MS (ESI) found: 473 [M+H]⁺. ¹H NMR (400 MHz, CD₃OD) δ 9.40 (s, 1H) 8.11 (s, 1H), 8.00 (s, 1H), 4.36 (td, J=10.6, 5.2 Hz, 1H), 4.22-4.09 (m, 1H), 4.01 (s, 1H), 3.93 (d, J=3.1 Hz, 1H), 3.80 (dd, J=12.9, 4.0 Hz, 1H), 3.73-3.59 (m, 2H), 3.13 (s, 1H).

### Preparation of (2R,3R,4R,5S)-2-(azocan-1-ylmethyl)-5-((6-(trifluoromethyl)pyrazin-2-yl)amino)tetrahydro-2H-pyran-3,4-diol (A73)

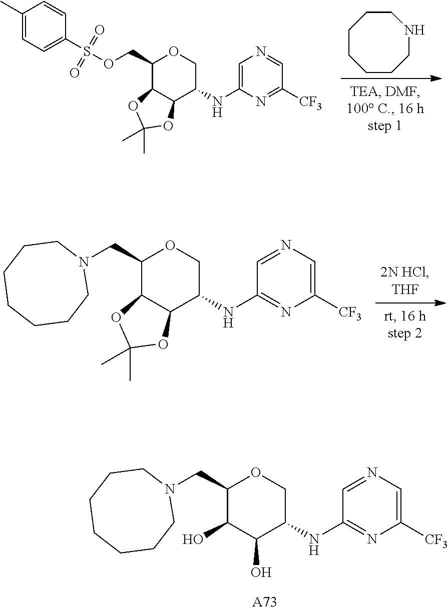

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (100 mg, 0.19 mmol) in DMF (5 mL) was added azocane (0.25 mL) and TEA (2 mL), then the mixture was stirred at 100° C. for 16 h. After completion, the mixture was quenched with 10 mL water and extracted with EA (10 mL×3), organic layers were combined and dried over anhydrous Na₂SO₄ and concentrated under vacuum to give crude N-((3aS,4R,7S,7aR)-4-(azocan-1-ylmethyl)-2,2-dimethyltetrahydro-4H-[1,3]dioxolo[4,5-c]pyran-7-yl)-6-(trifluoromethyl)pyrazin-2-amine as yellow gel which used to next step directly. LC-MS (ESI) found: 445 [M+H]⁺.
Step 2: To a solution of N-((3aS,4R,7S,7aR)-4-(azocan-1-ylmethyl)-2,2-dimethyltetrahydro-4H-[1,3]dioxolo[4,5-c]pyran-7-yl)-6-(trifluoromethyl)pyrazin-2-amine (crude) in THF (5 mL) was added 2 N HCl/H₂O (2 mL), then the mixture was stirred at an ambient temperature for 16 h. After completion, NH₄₀H was added to the reaction mixture to adjust the pH to 7, and mixture was concentrated and purified by reversed phase flash column (30% MeCN in water) to give (2R,3R,4R,5S)-2-(azocan-1-ylmethyl)-5-((6-(trifluoromethyl)pyrazin-2-yl)amino)tetrahydro-2H-pyran-3,4-diol as yellow solid (32 mg, yield: 40%). LC-MS (ESI) found: 405 [M+H]⁺. ¹H NMR (400 MHz, CD₃OD) δ 8.11 (s, 1H), 7.99 (s, 1H), 4.34 (td, J=10.6, 5.2 Hz, 1H), 4.11 (dd, J=11.0, 5.2 Hz, 1H), 3.98 (d, J=2.7 Hz, 1H), 3.64 (dd, J=10.5, 3.2 Hz, 1H), 3.50 (t, J=5.8 Hz, 1H), 3.10 (t, J=10.8 Hz, 1H), 2.87 (dd, J=13.3, 5.6 Hz, 1H), 2.74 (d, J=5.0 Hz, 5H), 1.63 (s, 10H).

### Preparation of N-(((2R,3R,4R,5S)-3,4-dihydroxy-5-((6-(trifluoromethyl)pyrazin-2-yl)amino)tetrahydro-2H-pyran-2-yl)methyl)picolinamide (A74)

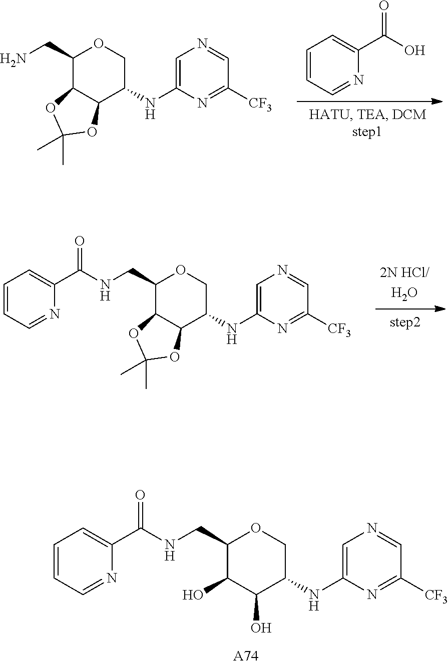

Step 1: To solution of picolinic acid (20 mg, 0.072 mmol) in DMF (5 mL) were added DIEA (37 mg, 0.144 mmol) HATU (65 mg, 0.171 mmol) at an ambient temperature. After stirring 20 min, N-[(3aS,4R,7S,7aR)-4-(aminomethyl)-2,2-dimethyl-hexahydro-[1,3]dioxolo[4,5-c]pyran-7-yl]-6-(trifluoromethyl)pyrazin-2-amine (25 mg, 0.072 mmol) was added. The mixture was stirred at an ambient temperature overnight, then concentrated. The residue was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)picolinamide (12 mg, 0.026 mmol, 36.88%) as pale yellow oil. LC-MS (ESI) found: 454 [M+H]⁺.
Step 2: To solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)picolinamide (12 mg, 0.026 mmol) in THF (3 mL) were added HCl/H₂O (0.5 mL) The mixture was stirred at an ambient temperature for 1 h. The pH of the reaction mixture was adjusted to 7 and concentrated. The residue was purified by Prep-HPLC (MeCN/H₂O, 0.1% formic acid) to give N-(((2R,3R,4R,5S)-3,4-dihydroxy-5-((6-(trifluoromethyl)pyrazin-2-yl)amino)tetrahydro-2H-pyran-2-yl)methyl)picolinamide acid as white solid (10 mg, yield: 91%). LC-MS (ESI) found: 414 [M+H]⁺. ¹H NMR (400 MHz, CD₃OD) δ 8.63 (d, J=4.1 Hz, 1H), 8.10 (d, J 5.9 Hz, 2H), 8.00 (s, 1H) 7.96 (td, J=7.7, 1.7 Hz, 1H), 7.55 (ddd, J=7.6, 4.8, 1.2 Hz, 1H), 4.37 (td, J=10.6, 5.1 Hz, 1H), 4.15 (dd, J=11.0, 5.2 Hz, 1H), 3.91 (d, J=3.1 Hz, 1H), 3.76 (dd, J=12.5, 4.1 Hz, 1H), 3.64 (ddd, J=18.2, 10.0, 4.1 Hz, 3H), 3.14 (t, J=10.9 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5-phenylpyrimidine-2-carboxamide(A75)

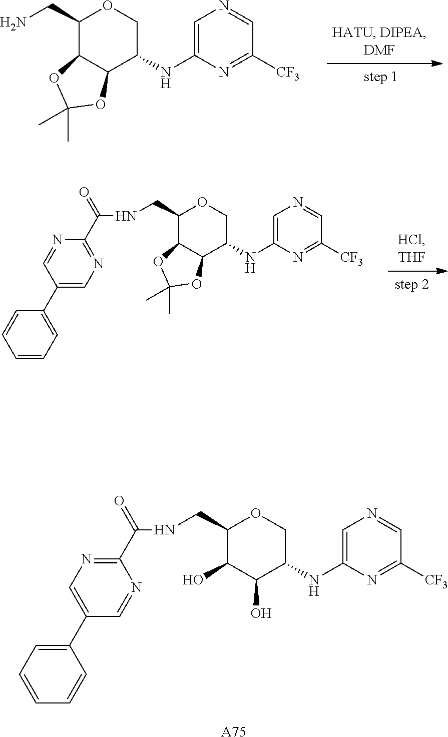 [SMILES: CC1(C)O[C@@H]2[C@H](O1)[C@@H](Nc1cncc(C(F)(F)F)n1)CO[C@@H]2CN ; CC1(C)O[C@@H]2[C@H](O1)[C@@H](Nc1cncc(C(F)(F)F)n1)CO[C@@H]2CNC(=O)c1ncc(-c2ccccc2)cn1 ; O=C(NC[C@H]1OC[C@H](Nc2cncc(C(F)(F)F)n2)[C@@H](O)[C@H]1O)c1ncc(-c2ccccc2)cn1 ; "A75"]

Step 1: To a solution of 4-(methoxycarbonyl)cyclohexane-1-carboxylic acid (27 mg, 0.14 mmol) in DMF (2 mL) were added HATU (65 mg, 0.17 mmol) and DIEA (30 mg, 0.0.23 mmol), then the mixture was stirred at 0° C. for 0.5 h. Then 5-phenylpyrimidine-2-carboxylic acid (40 mg, 0.12 mmol) in DMF (0.5 ml) was added. The mixture was stirred at an ambient temperature for 1.5 h. The reaction mixture was purified by Prep-HPLC(MeCN/H₂O) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-5-phenylpyrimidine-2-carboxamide (30 mg, yield: 49%). LC-MS (ESI) found: 531 [M+H]⁺.
Step 2: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-5-phenylpyrimidine-2-carboxamide (30 mg, 0.06 mmol) in THF (3 ml) was added HCl/H₂O (0.5 ml, 2 N), then the mixture was stirred at an ambient temperature overnight. The pH of the reaction mixture was adjusted to 7 and concentrated. The residue was purified by Prep-HPLC (MeCN/H₂O, 0.1% formic acid) to give N-(((2R,3R,4R,5S)-3,4-dihydroxy-5-((6-(trifluoromethyl)pyrazin-2-yl)amino)tetrahydro-2H-pyran-2-yl)methyl)-5-phenylpyrimidine-2-carboxamide (4.7 mg, yield: 17%). LC-MS (ESI) found: 491 [M+H]⁺. ¹H NMR (400 MHz, DMSO-d₆): δ 9.30 (s, 2H), 8.90 (s, 1H), 8.22 (s, 1H), 8.08 (s, 1H), 7.95-7.88 (m, 2H), 7.60 (ddd, J=22.1, 17.1, 7.2 Hz, 4H), 4.99 (d, J=4.8 Hz, 1H), 4.85 (d, J=6.7 Hz, 1H), 4.11 (s, 1H), 3.93 (d, J=5.7 Hz, 1H), 3.83-3.71 (m, 1H), 3.67-3.44 (m, 4H), 3.00 (s, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5,6,7,8-tetrahydro-1,7-naphthyridine-3-carboxamide (A76)

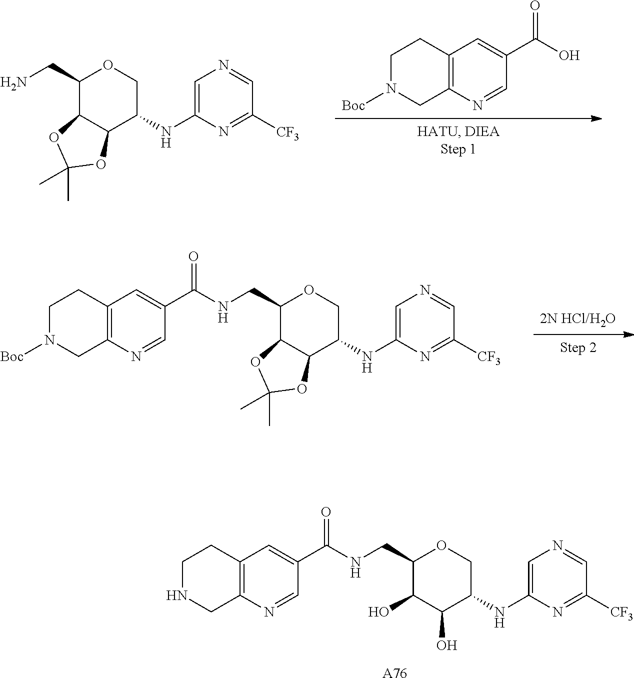 [SMILES: CC1(C)O[C@@H]2[C@H](O1)[C@@H](Nc1cncc(C(F)(F)F)n1)CO[C@@H]2CN ; CC(C)(C)OC(=O)N1CCc2cc(C(=O)O)cnc2C1 ; CC(C)(C)OC(=O)N1CCc2cc(C(=O)NC[C@H]3OC[C@H](Nc4cncc(C(F)(F)F)n4)[C@H]4OC(C)(C)O[C@H]43)cnc2C1 ; O=C(NC[C@H]1OC[C@H](Nc2cncc(C(F)(F)F)n2)[C@@H](O)[C@H]1O)c1cnc2c(c1)CCNC2 ; "A76"]

Step 1: To a solution of 7-(tert-butoxycarbonyl)-5,6,7,8-tetrahydro-1,7-naphthyridine-3-carboxylic acid (48 mg, 0.17 mmol) in DMF (3 mL) were added HATU (65 mg, 0.17 mmol) and DIEA (37 mg, 029 mmol), then the mixture was stirred at 0° C. for 0.5 h. Then N-((3aS,4R,7S,7aR) 4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol) in DMF (0.5 ml) was added. The mixture was stirred at an ambient temperature overnight. The reaction mixture was purified by flash chromatography to give tert-butyl 3-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-5,8-dihydro-1,7-naphthyridine-7(6H)-carboxylate as pale yellow oil (60 mg, yield: 71%). LC-MS (ESI) found: 609 [M+H]⁺.
Step 2: To a solution of tert-butyl 3-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-5,8-dihydro-1,7-naphthyridine-7(6H)-carboxylate (60 mg, 0.10 mmol) in THF (3 ml), then 2N HCl/H₂O (0.5 mL) was added. The mixture was stirred at an ambient temperature for 6 h. Then, the pH of the reaction mixture was adjusted to 7 with NH₃·H₂O. The reaction mixture was purified by Prep-HPLC (MeCN in water from 5% to 70%, HCOOH) to give N-(((2R,3R,4R,5S)-3,4-dihydroxy-5-((6-(trifluoromethyl)pyrazin-2-yl)amino)tetrahydro-2H-pyran-2-yl)methyl)-5,6,7,8-tetrahydro-1,7-naphthyridine-3-carboxamide as white solid (20 mg, yield: 43%). LC-MS (ESI) found: 468 [M+H]⁺. ¹H NMR (400 MHz, CD₃OD): δ 8.84 (d, J=2.0 Hz, 1H), 8.45 (s, 1H), 8.11 (s, 1H), 8.08 (d, J=1.9 Hz, 1H), 8.00 (s, 1H), 4.35 (d, J=4.7 Hz, 3H) 4.14 (dd, J=11.0, 5.1 Hz, 1H), 3.91 (d, J=2.9 Hz, 1H), 3.68 (dd, J=13.9, 5.6 Hz, 3H), 3.55 (dd, J=12.6, 7.9 Hz, 1H), 3.46 (d, J=5.8 Hz, 2H), 3.16-3.10 (m, 3H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5,6,7,8-tetrahydro-1,6-naphthyridine-2-carboxamide (A77)

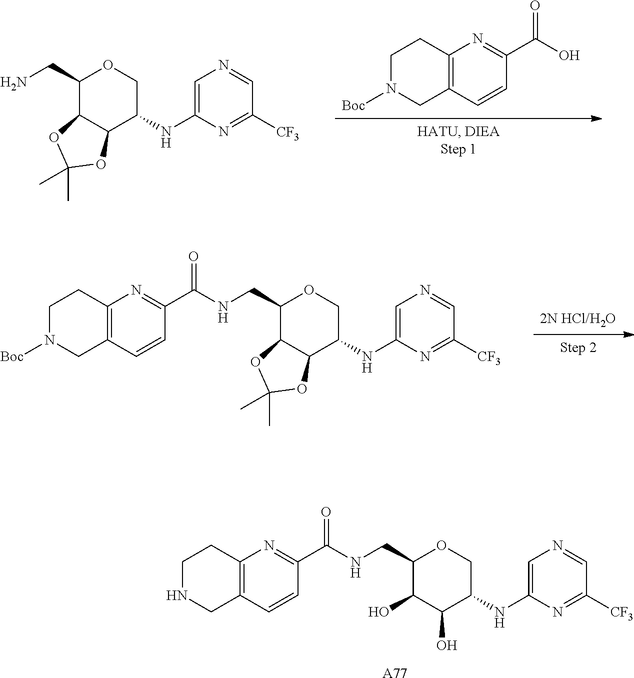 [SMILES: CC1(C)O[C@@H]2[C@H](O1)[C@@H](Nc1cncc(C(F)(F)F)n1)CO[C@@H]2CN ; CC(C)(C)OC(=O)N1CCc2nc(C(=O)O)ccc2C1 ; CC(C)(C)OC(=O)N1CCc2nc(C(=O)NC[C@H]3OC[C@H](Nc4cncc(C(F)(F)F)n4)[C@H]4OC(C)(C)O[C@H]43)ccc2C1 ; O=C(NC[C@H]1OC[C@H](Nc2cncc(C(F)(F)F)n2)[C@@H](O)[C@H]1O)c1ccc2c(n1)CCNC2 ; "A77"]

Step 1: To a solution of 6-(tert-butoxycarbonyl)-5,6,7,8-tetrahydro-1,6-naphthyridine-2-carboxylic acid (48 mg, 0.17 mmol) in DMF (3 mL) were added HATU (65 mg, 0.17 mmol) and DIEA (37 mg, 0.29 mmol), then the mixture was stirred at 0° C. for 0.5 h. Then N-((3aS,4R,7S,7aR)-4-(aminomethyl)-2,2-dimethyltetrahydro-4H-[1,3]dioxolo[4,5-c]pyran-7-yl) 6-(trifluoromethyl)pyrazin-2-amine (50 mg, 0.14 mmol) in DMF (0.5 ml) was added. The mixture was stirred at an ambient temperature overnight. The reaction mixture was purified by flash chromatography to give tert-butyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-7,8-dihydro-1,6-naphthyridine-6(5H)-carboxylate as pale yellow oil (30 mg, yield: 35%). LC-MS (ESI) found: 609 [M+H]⁺.
Step 2: To a solution of tert-butyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-7,8-dihydro-1,6-naphthyridine-6(5H)-carboxylate (30 mg, 0.05 mmol) in DCM (5 ml), then TFA (0.5 mL) was added. The mixture was stirred at an ambient temperature for 6 h. Then, the pH of the reaction mixture was adjusted to 7 with NH₃·H₂O. The reaction mixture was purified by Prep-HPLC (MeCN in water from 5% to 70%) to give N-(((2R,3R,4R,5S)-3,4-dihydroxy-5-((6-(trifluoromethyl)pyrazin-2-yl)amino)tetrahydro-2H-pyran-2-yl)methyl)-5,6,7,8-tetrahydro-1,6-naphthyridine-2-carboxamide as white solid (10 mg, yield: 22%). LC-MS (ESI) found: 468 [M+H]⁺. ¹H NMR (400 MHz, CD₃OD): δ 8.11 (s, 1H), 8.00 (d, J=3.0 Hz, 1H), 7.98 (d, J=8.1 Hz, 1H), 7.79 (d, J=8.1 Hz, 1H), 4.40 (s, 2H), 4.39-4.31 (m, 1H), 4.15 (dd, J=11.0, 5.2 Hz, 1H), 3.91 (d, J=3.1 Hz, 1H), 3.76 (dd, J=13.1, 4.1 Hz, 1H), 3.70-3.62 (m, 2H), 3.61-3.54 (m, 3H), 3.24 (t, J=6.2 Hz, 2H), 3.12 (t, J=10.9 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-7H-pyrrolo[2,3-d]pyrimidine-2-carboxamide (A78)

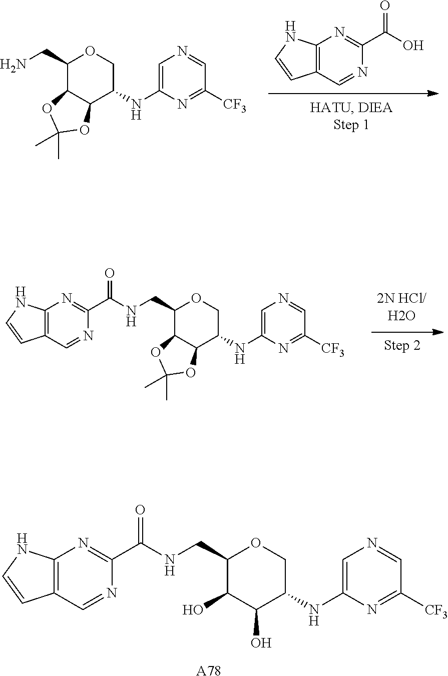 [SMILES: CC1(C)O[C@@H]2[C@H](O1)[C@@H](Nc1cncc(C(F)(F)F)n1)CO[C@@H]2CN ; O=C(O)c1ncc2cc[nH]c2n1 ; CC1(C)O[C@@H]2[C@H](O1)[C@@H](Nc1cncc(C(F)(F)F)n1)CO[C@@H]2CNC(=O)c1ncc2cc[nH]c2n1 ; O=C(NC[C@H]1OC[C@H](Nc2cncc(C(F)(F)F)n2)[C@@H](O)[C@H]1O)c1ncc2cc[nH]c2n1 ; "A78"]

Step 1: To a solution of 7H-pyrrolo[2,3-d]pyrimidine-2-carboxylic acid (23.4 mg, 0.2 mmol) in DMF (3 mL) was added HATU (54.6 mg, 0.2 mmol) and DIEA (0.1 mL, 0.5 mmol) at an ambient temperature. The solution was stirred at an ambient temperature for 1 h. N-[(3aS,4R,7S,7aR)-4-(aminomethyl)-2,2-dimethyl-hexahydro-[1,3]dioxolo[4,5-c]pyran-7-yl]-6-(trifluoromethyl)pyrazin-2-amine (50 mg, 0.2 mmol) was added. The mixture was stirred at an ambient temperature for 18 h. Then concentrated and the residue was purified by silica gel column with DCM/MeOH=10/1 to give N-{[(3aS,4R,7S,7aR)-2,2-dimethyl-7-{[6-(trifluoromethyl)pyrazin-2-yl]amino}-hexahydro-[1,3]dioxolo[4,5-c]pyran-4-yl]methyl}-7H-pyrrolo[2,3-d]pyrimidine-2-carboxamide (29.8 mg, 0.06 mmol, 42% yield) as yellow oil. LC-MS (ESI) found: 494.2 [M+H]⁺.
Step 2: To a solution of N-{[(3aS,4R,7S,7aR)-2,2-dimethyl-7-{[6-(trifluoromethyl)pyrazin-2-yl]amino}-hexahydro-[1,3]dioxolo[4,5-c]pyran-4-yl]methyl}-7H-pyrrolo[2,3-d]pyrimidine-2-carboxamide (30 mg, 0.06 mmol) in THF (5 mL) was added HCl (5 mL, 10.0 mmol) at an ambient temperature. The reaction was stirred at an ambient temperature for 18 h. Then pH was adjusted to 7 with NaHCO₃, and extracted with ethyl acetate. The organic layer was dried over Na₂SO₄, concentrated and purified by reverse phase chromatography with MeOH/H₂O to give N-(((2R,3R,4R,5S)-3,4-dihydroxy-5-((6-(trifluoromethyl)pyrazin-2-yl)amino)tetrahydro-2H-pyran-2-yl)methyl)-7H-pyrrolo[2,3-d]pyrimidine-2-carboxamide (10 mg, 0.02 mmol, 36% yield) as white solid. LC-MS (ESI) found: 454.3 [M+H]⁺. ¹H NMR (400 MHz, DMSO-d₆) δ 12.46 (s, 1H) 9.11 (s, 1H), 8.80 (d, J=6.0 Hz, 1H), 8.22 (s, 1H), 8.07 (s, 1H), 7.77 (d, J=3.5 Hz, 1H), 7.67 (d, J=7.4 Hz, 1H), 6.71 (d, J=3.5 Hz, 1H), 5.00 (d, J=4.7 Hz, 1H), 4.87 (d, J=6.4 Hz, 1H), 4.05-4.15 (m, 1H), 3.94 (dd, J=10.9, 5.0 Hz, 1H), 3.77 (t, J=3.6 Hz, 1H), 3.45-3.63 (m, 4H), 3.00 (t, J=10.7 Hz, 1H).

### Preparation of N-(((2R,3R,4R,5S)-3,4-dihydroxy-5-((6-(trifluoromethyl) pyrazin-2-yl) amino) tetrahydro-2H-pyran-2-yl) methyl)-3H-imidazo[4,5-b]pyridine-6-carboxamide (A79)

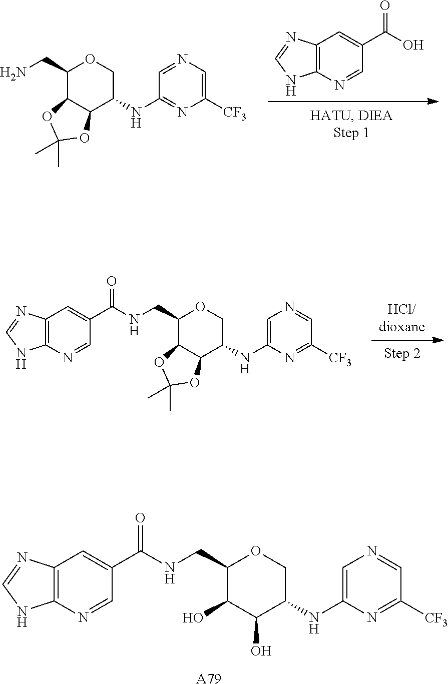 [SMILES: CC1(C)O[C@@H]2[C@H](O1)[C@@H](Nc1cncc(C(F)(F)F)n1)CO[C@@H]2CN ; O=C(O)c1cnc2[nH]cnc2c1 ; CC1(C)O[C@@H]2[C@H](O1)[C@@H](Nc1cncc(C(F)(F)F)n1)CO[C@@H]2CNC(=O)c1cnc2[nH]cnc2c1 ; O=C(NC[C@H]1OC[C@H](Nc2cncc(C(F)(F)F)n2)[C@@H](O)[C@H]1O)c1cnc2[nH]cnc2c1 ; "A79"]

Step 1: A solution of 3H-imidazo[4,5-b]pyridine-6-carboxylic acid (25.76 mg, 0.16 mmol), DIEA (55.60 mg, 0.43 mmol) and HATU (60.06 mg, 0.16 mmol) in DMF (3 mL) was stirred at an ambient temperature for 30 min. N-((3aS,4R,7S,7aR)-4-(aminomethyl)-2,2-dimethyltetrahydro-4H-[1,3]dioxolo[4,5-c]pyran-7-yl)-6-(trifluoromethyl) pyrazin-2-amine (50 mg, 0.14 mmol) was added at an ambient temperature. The reaction was stirred overnight. The resulting mixture was concentrated in vacuo. The crude product was purified by flash chromatography (silica gel, 0˜10% MeOH in DCM) to give N-(((3aS,4R,7S,7aR)-2,2-dimethyl-7-((6-(trifluoromethyl) pyrazin-2-yl) amino) tetrahydro-4H-[1,3]dioxolo[4,5-c]pyran-4-yl)methyl)-3H-imidazo[4,5-b]pyridine-6-carboxamide (50 mg, 70.59% yield) as a colorless oil. LC-MS (ESI) found: 494 [M+H]⁺.
Step 2: To a solution of N-(((3aS,4R,7S,7aR)-2,2-dimethyl-7-((6-(trifluoromethyl) pyrazin-2-yl) amino) tetrahydro-4H-[1,3]dioxolo[4,5-c]pyran-4-yl) methyl)-3H-imidazo[4,5-b]pyridine-6-carboxamide (50 mg, 0.101 mmol) in HCl/dioxane (5 mL, 4 N) was stirred at an ambient temperature for 2 h. The pH of the mixture was adjusted to ˜7-8 with NH₃·H₂O. The resulting mixture was purified by pre-HPLC (0.1% NH₃·H₂O) to give N-(((2R,3R,4R,5S)-3,4-dihydroxy-5-((6-(trifluoromethyl) pyrazin-2-yl) amino) tetrahydro-2H-pyran-2-yl) methyl)-3H-imidazo[4,5-b]pyridine-6-carboxamide (27.3 mg, 59.42% yield) as a white solid. LC-MS (ESI) found: 454 [M+H]⁺. ¹H NMR (400 MHz, CD₃OD) δ 8.10 (s, 1H), 8.00 (s, 1H), 4.31 (s, 1H), 4.11 (dd, J=11.0, 5.3 Hz, 1H), 3.85 (s, 1H), 3.64 (d, J=10.6 Hz, 1H), 3.51 (d, J=10.4 Hz, 2H), 3.27-3.23 (m, 1H) 3.09 (dd, J=21.4, 10.5 Hz, 1H), 1.95 (s, 5H), 1.74 (d, J=12.5 Hz, 3H), 1.66 (d, J=16.1 Hz, 9H).

### Preparation of N-(((2R,3R,4R,5S)-3,4-dihydroxy-5-((6-(trifluoromethyl)pyrazin-2-yl)amino)tetrahydro-2H-pyran-2-yl)methyl)isonicotinamide(A80)

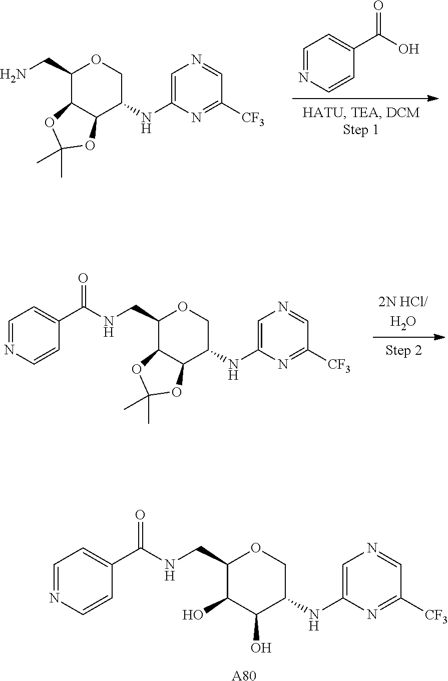

Step 1: To solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.144 mmol) in DMF (5 mL) were added DIEA (37 mg, 0.144 mmol) HATU (65 mg, 0.171 mmol) at an ambient temperature. After stirring 20 min, N-[(3aS,4R,7S,7aR)-4-(aminomethyl)-2,2-dimethyl-hexahydro-[1,3]dioxolo[4,5-c]pyran-7-yl]-6-(trifluoromethyl)pyrazin-2-amine (50 mg, 0.144 mmol) was added. The mixture was stirred at an ambient temperature overnight, then concentrated. The residue was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)isonicotinamide (43.1 mg, 0.095 mmol, 66.22%) as pale yellow oil. LC-MS (ESI) found: 454 [M+H]⁺.
Step 2: To solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)isonicotinamide (43.1 mg, 0.095 mmol, 66.22%) in THF (3 mL) were added HCl (0.5 mL) The mixture was stirred at an ambient temperature for 1 h. Then 2N HCl/H₂O (6 ml) was added. The pH of the reaction mixture was adjusted to 7 and concentrated. The residue was purified by Prep-HPLC (MeCN/H₂O, 0.1% formic acid) to give N-(((2R,3R,4R,5S)-3,4-dihydroxy-5-((6-(trifluoromethyl)pyrazin-2-yl)amino)tetrahydro-2H-pyran-2yl)methyl)isonicotinamide (31 mg, yield: 83.3%). LC-MS (ESI) found: 414 [M+H]⁺. ¹H NMR (400 MHz, CD₃OD) δ 8.70 (s, 2H), 8.11 (s, 1H), 8.00 (s, 1H), 7.80 (d, J=5.9 Hz, 2H), 4.36 (td, J=10.6, 5.1 Hz, 1H), 4.15 (dd, J=11.0, 5.2 Hz, 1H), 3.92 (d, J=3.0 Hz, 1H), 3.73 (dd, J=13.2, 4.4 Hz, 1H), 3.70-3.65 (m, 2H), 3.55 (dd, J=13.2, 7.6 Hz, 1H) 3.12 (t, J=10.9 Hz, 1H).

### Preparation of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinazoline-2-carboxamide (A81)

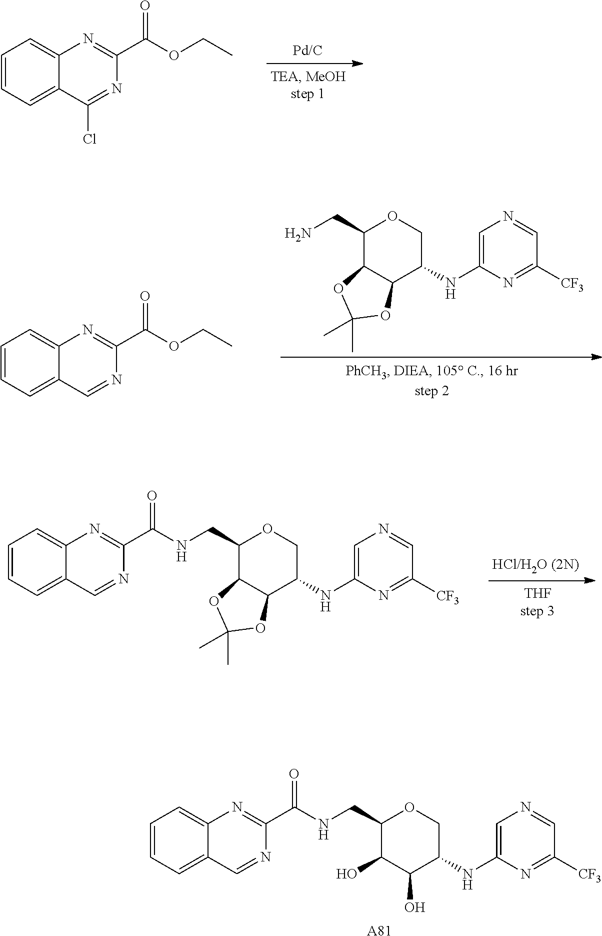

Step 1: To a solution of ethyl 4-chloroquinazoline-2-carboxylate (1.0 g, 4.23 mmol) in MeOH (20.0 mL) at the room temperature was added TEA (214 mg, 2.12 mmol), Pd/C (100.0 mg, 10%), the mixture was stirred at the room temperature for 0.5 h. The mixture was filtered, concentrated under reduced pressure to give ethyl quinazoline-2-carboxylate (888.8 mg, 4.40 mmol, 80%). LC-MS (ESI) found: 203 [M+H]⁺.
Step 2: To a solution of ethyl quinazoline-2-carboxylate (888.8 mg, 4.40 mmol) in PhCH3 (20.0 mL) was added DIEA (1.7 g, 13.2 mmol), HATU (1.67 g, 4.40 mmol), The mixture was stirred at 105° C. for 16 h. The mixture was concentrated under reduced pressure to give a crude product, which was purified by column (PE/EA=1/1) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinazoline-2-carboxamide (765.0 mg, 1.5 mmol, 34.0%). LC-MS (ESI) found: 505 [M+H]⁺.
Step 3: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)quinazoline-2-carboxamide (765.0 mg, 1.5 mmol) in THF (20.0 mL) was added HCl/H₂O (20.0 mL, 3N), the mixture was stirred at the room temperature for 16 h. The mixture was quenched by NH₃·H₂O to adjust the pH to 7.0 and concentrated under reduced pressure to give a crude product, which was purified by column chromatography to give N-(((2R,3R,4R,5S)-3,4-dihydroxy-5-((6-(trifluoromethyl)pyrazin-2-yl)amino)tetrahydro-2H-pyran-2-yl)methyl)quinazoline-2-carboxamide (232.0 mg, 0.5 mmol, 33.3%). LC-MS (ESI) found: 465 [M+H]⁺. ¹H NMR (400 MHz, CD₃OD) δ 9.65 (s, 1H), 8.31-8.16 (m, 2H), 8.15-8.05 (m, 2H), 8.00 (s, 1H), 7.91-7.78 (m, 1H), 4.38 (td, J=10.6, 5.1 Hz, 1H), 4.17 (dd, J=11.0, 5.2 Hz, 1H), 3.97 (d, J=3.1 Hz, 1H) 3.86 (dd, J=13.0, 4.2 Hz, 1H), 3.77-3.62 (m, 3H), 3.15 (t, J=10.9 Hz, 1H). 19F NMR (377 MHz, CD₃OD) δ −70.31 (s).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5H-pyrrolo[2,3-b]pyrazine-3-carboxamide (A82)

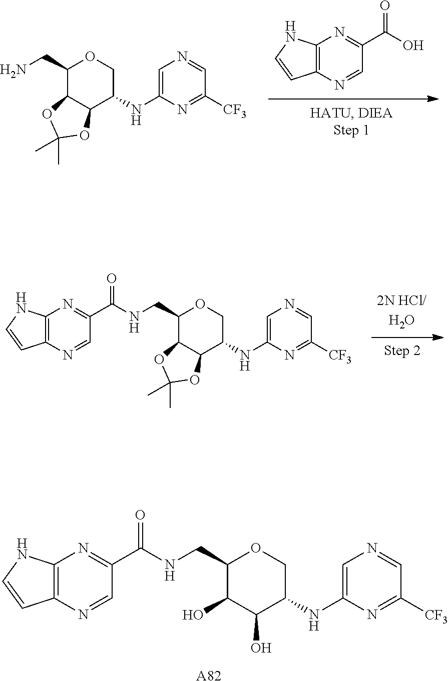 [SMILES: CC1(C)O[C@@H]2[C@H](O1)[C@@H](Nc1cncc(C(F)(F)F)n1)CO[C@@H]2CN ; O=C(O)c1cnc2cc[nH]c2n1 ; CC1(C)O[C@@H]2[C@H](O1)[C@@H](Nc1cncc(C(F)(F)F)n1)CO[C@@H]2CNC(=O)c1cnc2cc[nH]c2n1 ; O=C(NC[C@H]1OC[C@H](Nc2cncc(C(F)(F)F)n2)[C@@H](O)[C@H]1O)c1cnc2cc[nH]c2n1 ; "A82"]

Step 1: To a solution of 5H-pyrrolo[2,3-b]pyrazine-3-carboxylic acid (23.4 mg, 0.2 mmol) in DMF (3 mL) was added HATU (54.6 mg, 0.2 mmol) and DIEA (0.1 mL, 0.5 mmol) at an ambient temperature. The solution was stirred at an ambient temperature for 1 h. N-[(3aS,4R,7S,7aR)-4-(aminomethyl)-2,2-dimethyl-hexahydro-[1,3]dioxolo[4,5-c]pyran-7-yl]-6-(trifluoromethyl)pyrazin-2-amine (50 mg, 0.2 mmol) was added. The mixture was stirred at an ambient temperature for 18 h. Then concentrated and the residue was purified by silica gel column with DCM/MeOH=10/1 to give N-{[(3aS,4R,7S,7aR)-2,2-dimethyl-7-{[6-(trifluoromethyl)pyrazin-2-yl]amino}-hexahydro-[1,3]dioxolo[4,5-c]pyran-4-yl]methyl}-5H-pyrrolo[2,3-b]pyrazine-3-carboxamide (35 mg, 0.1 mmol, 49% yield) as yellow oil. LC-MS (ESI) found: 494.2 [M+H]⁺.
Step 2: To a solution of N-{[(3aS,4R,7S,7aR)-2,2-dimethyl-7-{[6-(trifluoromethyl)pyrazin-2-yl]amino}-hexahydro-[1,3]dioxolo[4,5-c]pyran-4-yl]methyl}-5H-pyrrolo[2,3-b]pyrazine-3-carboxamide (35 mg, 0.1 mmol) in THF (5 mL) was added HCl/H₂O (5 mL, 10.0 mmol) at an ambient temperature. The reaction was stirred at an ambient temperature for 18 h. Then pH was adjusted to 7 with NaHCO₃, and extracted with ethyl acetate. The organic layer was dried over Na₂SO₄, filtered and concentrated and purified by reverse phase with MeOH/H₂O to give N-(((2R,3R,4R,5S)-3,4-dihydroxy-5-((6-(trifluoromethyl)pyrazin-2-yl)amino)tetrahydro-2H-pyran-2-yl)methyl)-5H-pyrrolo[2,3-b]pyrazine-3-carboxamide (22 mg, 0.05 mmol, 69% yield) as white solid. LC-MS (ESI) found: 454.3 [M+H]⁺. ¹H NMR (400 MHz, DMSO-d₆) δ 12.31 (s, 1H), 9.04 (s, 1H), 8.50 (t, J=5.7 Hz, 1H), 8.22 (s, 1H), 8.13 (d, J=3.6 Hz, 1H), 8.08 (s, 1H), 7.66 (d, J=7.5 Hz, 1H), 6.76 (d, J=3.6 Hz, 1H), 5.02 (d, J=4.7 Hz, 1H), 4.86 (d, J=6.7 Hz, 1H), 4.16-4.05 (m, 1H), 3.94 (dd, J=10.8, 5.0 Hz, 1H), 3.80-3.75 (m, 1H), 3.52-3.62 (m, 4H), 3.00 (t, J=10.8 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5-phenylpyrazine-2-carboxamide (A83)

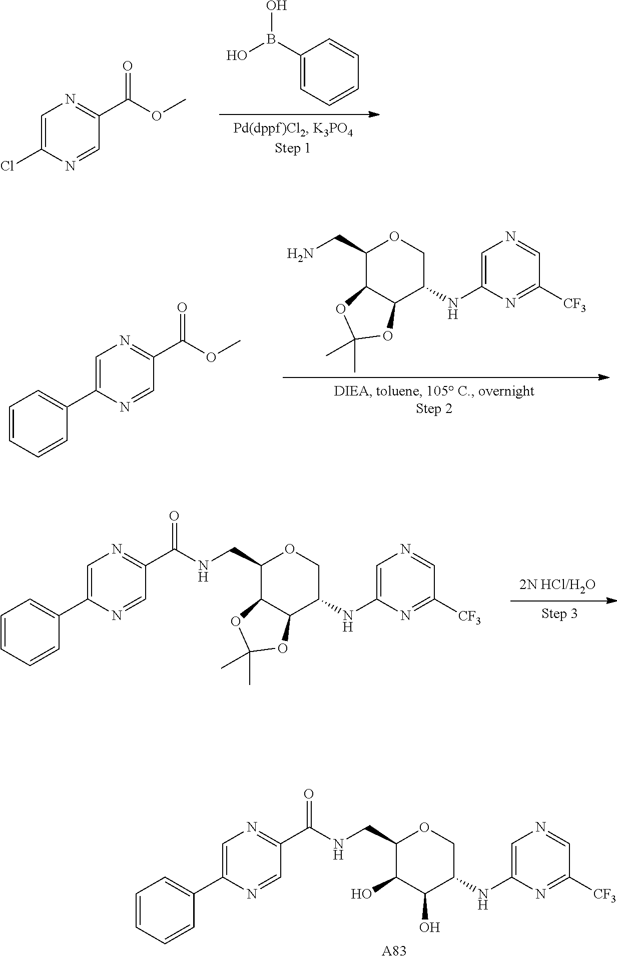

Step 1: To solution of methyl 5-chloropyrazine-2-carboxylate (500 mg, 2.864 mmol) in dioxane-water (12 mL) were added K₂CO₃ (1200 mg, 2.864 mmol) phenylboronic acid (387 mg, 3.174 mmol) Pd(dppf)C12 (423 mg, 2.864 mmol) at 88° C. The mixture was stirred at an ambient temperature overnight. The residue was purified by flash chromatography (silica gel, 0-50% PE in EA) to give methyl 5-phenylpyrazine-2-carboxylate (472 mg, 2.183 mmol, 76.22%). LC-MS (ESI) found: 215 [M+H]⁺.
Step 2: To a solution of methyl 5-phenylpyrazine-2-carboxylate (50 mg, 0.233 mmol) in Toluene (2 mL) was added N-[(3aS,4R,7S,7aR)-4-(aminomethyl)-2,2-dimethyl-hexahydro-[1,3]dioxolo[4,5-c]pyran-7-yl]-6-(trifluoromethyl)pyrazin-2-amine (73.91 mg, 0.212 mmol) DIEA (0.035 mL, 0.212 mmol) at 105° C. for overnight. The residue was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-5-phenylpyrazine-2-carboxamide(90.1 mg, 0.17 mmol, 80%) LC-MS (ESI) found: 531 [M+H]⁺.
Step 3: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-5-phenylpyrazine-2-carboxamide (43.1 mg, 0.1 mmol, 66.2%) in THF (3 mL) was added 2N HCl/H₂O (6 ml). The reaction mixture was adjusted to pH=7 and concentrated. the residue was purified by Prep-HPLC (MeCN/H₂O, 0.1% FA) to give N-(((2R,3R,4R,5 S)-3,4-dihydroxy-5-((6-(trifluoromethyl)pyrazin-2-yl)amino)tetrahydro-2H-pyran-2-yl)methyl)-5-phenylpyrazine-2-carboxamide (31 mg, yield: 83.3%). ¹H NMR (400 MHz, CD₃OD) δ 9.27 (d, J=1.4 Hz, 1H), 9.17 (d, J=1.4 Hz, 1H), 8.20-8.15 (m, 2H), 8.11 (s, 1H), 8.00 (s, 1H), 7.58-7.51 (m, 3H), 4.37 (td, J=10.6, 5.2 Hz, 1H), 4.15 (dd, J=11.0, 5.2 Hz, 1H), 3.93 (s, 1H), 3.79 (dd, J=12.7, 4.1 Hz, 1H), 3.66 (d, J=6.9 Hz, 3H) 3.13 (s, 1H). LC-MS (ESI) found: 491 [M+H]⁺

### Preparation of 2-((((2R,3R,4R,5S)-3,4-dihydroxy-5-((6-(trifluoromethyl)pyrazin-2-yl)amino)tetrahydro-2H-pyran-2-yl)methyl)carbamoyl)pyrimidine-5-carboxylic acid (A84)

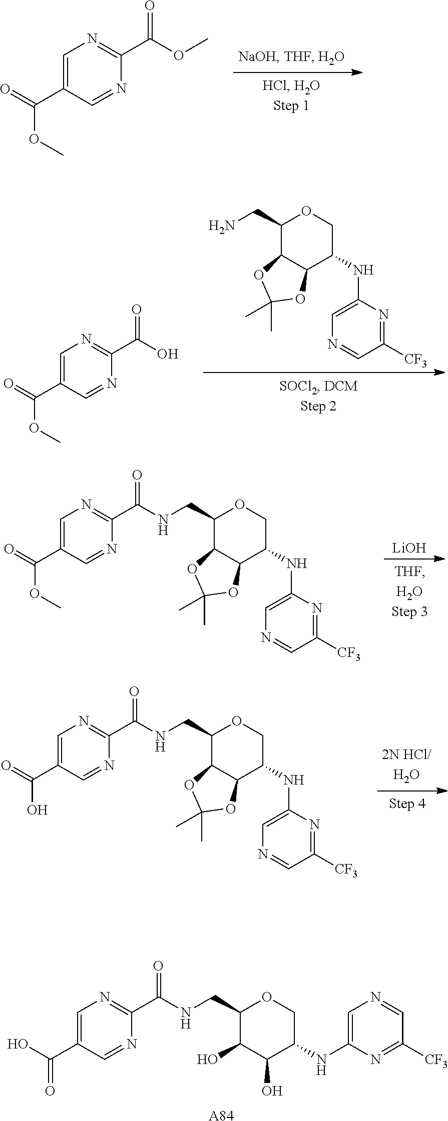

Step 1: To a solution of dimethyl pyrimidine-2,5-dicarboxylate (500 mg, 2.5 mmol) in MeOH (10 mL) was added NaOH (100 mg, 2.5 mmol). Then the mixture was stirred at 70° C. for 1 h. To the mixture was added 2 N HCl/H₂O (1.3 mL). The mixture was concentrated in vacuo. The crude was washed with DCM (3 mL) to give 5-(methoxycarbonyl)pyrimidine-2-carboxylic acid as yellow solid which was used in the next step without further purification. (417 mg, yield: 90%). LC-MS (ESI) found: 183 [M+H]⁺.
Step 2: A solution of 5-(methoxycarbonyl)pyrimidine-2-carboxylic acid (300 mg, 1.64 mmol) in SOCl₂ (5 mL) was stirred at 85° C. under N₂ overnight. The mixture was concentrated. The crude was dissolved in DCM (20 mL). To the mixture were added TEA (3 mL) and N-((3aS,4R,7S,7aR) 4-(aminomethyl)-2,2-dimethyltetrahydro-4H-[1,3]dioxolo[4,5-c]pyran-7-yl)-6-(trifluoromethyl)pyrazin-2-amine (600 mg, 1.72 mmol). The mixture was stirred at an ambient temperature under N₂ overnight. The mixture was concentrated in vacuo. The crude was purified by flash silica column chromatography (eluent=3% MeOH in DCM) to give methyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)pyrimidine-5-carboxylate (350 mg, yield: 41%) as yellow solid. LC-MS (ESI) found: 513 [M+H]⁺.
Step 3: To a solution of methyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)pyrimidine-5-carboxylate (350 mg, 0.68 mmol) in THF/H₂O (4 ml/1 mL) was added LiOH (114 mg, 4.7 mmol), then the mixture was stirred at an ambient temperature for 5 h. To the mixture were added water (1 mL) and 2 N HCl/H₂O (3 mL). The mixture was extracted with ethyl acetate (2 mL×3). The organic layers was dried over Na₂SO₄, concentrated in vacuo to give 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)pyrimidine-5-carboxylic acid (320 mg, yield: 94%) as yellow solid. LC-MS (ESI) found: 499 [M+H]⁺.
Step 4: To a solution of 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)pyrimidine-5-carboxylic acid (300 mg, 0.60 mmol) in THF (3 mL) was added 2 N HCl/H₂O (1.5 mL). The mixture was stirred at an ambient temperature for 4 h. The pH of the reaction mixture was adjusted to 7 and concentrated. The residue was purified by Prep-HPLC (MeCN/H₂O, 0.1% formic acid) to give 2-((((2R,3R,4R,5S)-3,4-dihydroxy-5-((6-(trifluoromethyl)pyrazin-2-yl)amino)tetrahydro-2H-pyran-2-yl)methyl)carbamoyl)pyrimidine-5-carboxylic acid as white solid (170 mg, yield: 61%). LC-MS (ESI) found: 459 [M+H]⁺. ¹H NMR (400 MHz, CD₃OD) δ 9.39 (s, 1H), 8.11 (s, 1H), 8.00 (s, 1H), 4.36 (td, J=10.6, 5.1 Hz, 1H), 4.15 (dd, J=11.0, 5.2 Hz, 1H), 3.94 (d, J=3.1 Hz, 1H), 3.80 (dd, J=12.9, 4.1 Hz, 1H), 3.72-3.60 (m, 2H), 3.13 (s, 1H).

### Preparation of (2R,3R,4R,5S)-2-(((1,3,5-triazin-2-yl)amino)methyl)-5-((6-(trifluoromethyl)pyrazin-2-yl)amino)tetrahydro-2H-pyran-3,4-diol(A85)

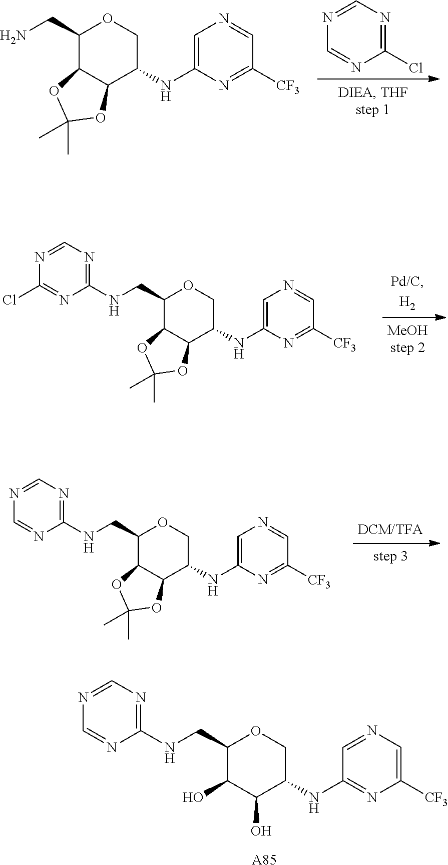

Step 1: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (100 mg, 0.28 mmol) in THE (3 mL) were added DIEA (74 mg, 0.57 mmol) and 2,4-dichloro-1,3,5-triazine (47 mg, 0.31 mmol). Then the mixture was stirred at an ambient temperature for 2 h. The mixture was quenched with water (3 mL). The mixture was extracted with ethyl acetate (2 mL×3). The organic layer was dried over Na₂SO₄ and concentrated in vacuo. The crude was purified by flash silica column chromatography (eluent=3% MeOH in DCM) to give 4-chloro-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1,3,5-triazin-2-amine as white solid (50 mg, yield: 37%). LC-MS (ESI) found: 462 [M+H]⁺.
Step 2: To a solution of 4-chloro-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1,3,5-triazin-2-amine (50 mg, 0.10 mmol) in MeOH (3 mL) were added Pd/C (10%, 15 mg), and then the mixture was stirred at an ambient temperature under H2 overnight. The mixture was filtered and the filtrate was concentrated to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1,3,5-triazin-2-amine (40 mg, crude) as yellow oil which was used to next step directly. LC-MS (ESI) found: 428 [M+H]⁺.
Step 3: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-1,3,5-triazin-2-amine (40 mg, 0.09 mmol) in DCM (4 ml) was added TFA (1 mL), and then the mixture was stirred at an ambient temperature for 5 h. The pH of the reaction mixture was adjusted to 7 and concentrated. The residue was purified by Prep-HPLC (MeCN/H₂O, 0.1% formic acid) to give (2R,3R,4R,5S)-2-(((1,3,5-triazin-2-yl)amino)methyl)-5-((6-(trifluoromethyl)pyrazin-2-yl)amino)tetrahydro-2H-pyran-3,4-diol as white solid (2 mg, yield: 5%). LC-MS (ESI) found: 388 [M+H]⁺. ¹H NMR (400 MHz, DMSO-d₆): δ 8.64 (s, 1H), 8.55 (s, 1H), 8.54 (s, 1H), 8.22 (s, 1H), 8.08 (s, 1H), 7.67 (d, J=7.3 Hz, 1H), 4.16-3.99 (m, 3H), 3.90 (dd, J=10.9, 5.0 Hz, 2H), 3.74 (d, J=3.0 Hz, 1H), 3.66-3.57 (m, 1H), 3.57-3.50 (m, 2H), 3.49-3.37 (m, 1H), 2.95 (t, J=10.7 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5,6,7,8-tetrahydropyrido[4,3-d]pyrimidine-2-carboxamide(A86)

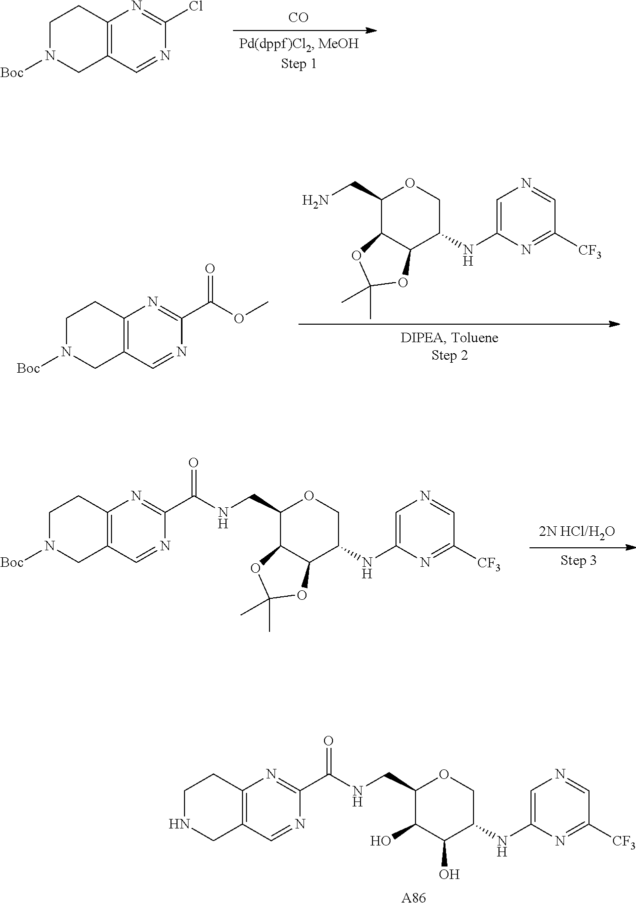

Step 1: A solution of tert-butyl 2-chloro-7,8-dihydropyrido[4,3-d]pyrimidine-6(5H)-carboxylate (800 mg, 2.97 mmol), Pd(dppf)Cl₂ (217 mg, 0.30 mmol) and TEA (3 mL) in MeOH (10 mL) was stirred at 80° C. under a CO balloon overnight. The reaction mixture was concentrated and the residue was purified by flash chromatography (Petroleum ether/ethyl acetate) to give 6-(tert-butyl) 2-methyl 7,8-dihydropyrido[4,3-d]pyrimidine-2,6(5H)-dicarboxylate (450 mg, yield: 52%). LC-MS (ESI) found: 294 [M+H]⁺.
Step 2: A solution of 6-(tert-butyl) 2-methyl 7,8-dihydropyrido[4,3-d]pyrimidine-2,6(5H)-dicarboxylate (55 mg, 0.19 mmol), N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (60 mg, 0.17 mmol) and DIPEA (3 mL) in toluene (5 mL) was stirred at 100° C. under a N2 balloon overnight. The reaction mixture was concentrated and the residue was purified by flash chromatography (DCM/MeOH) to give tert-butyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-7,8-dihydropyrido[4,3-d]pyrimidine-6(5H)-carboxylate (40 mg, yield: 35%). LC-MS (ESI) found: 610 [M+H]⁺.
Step 3: To a solution of tert-butyl 2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-7,8-dihydropyrido[4,3-d]pyrimidine-6(5H)-carboxylate (40 mg, 0.07 mmol) in DCM (3 mL) was added TFA (1 mL), and then the mixture was stirred at an ambient temperature overnight. The pH of the reaction mixture was adjusted to 7 and concentrated. The residue was purified by Prep-HPLC (MeCN/H₂O, 0.1% formic acid) to give N-(((2R,3R,4R,5S) 3,4-dihydroxy-5-((6-(trifluoromethyl)pyrazin-2-yl)amino)tetrahydro-2H-pyran-2-yl)methyl) 5,6,7,8-tetrahydropyrido[4,3-d]pyrimidine-2-carboxamide (10 mg, yield: 33%). LC-MS (ESI) found: 470 [M+H]⁺. ¹H NMR (400 MHz, DMSO-d₆): δ 8.75 (t, J=5.5 Hz, 1H), 8.60 (s, 1H), 8.22 (s, 1H), 8.07 (s, 1H), 7.66 (d, J=7.2 Hz, 1H), 4.97 (d, J=4.7 Hz, 1H), 4.85 (s, 1H), 4.09 (s, 1H) 3.95-3.87 (m, 3H), 3.73 (s, 1H), 3.61-3.41 (m, 5H), 3.05 (t, J=5.9 Hz, 2H), 2.96 (d, J=10.7 Hz, 1H), 2.83 (t, J=5.7 Hz, 2H).

### Preparation of 3,3′-((2-(6-azidohexanamido)-2-(1-((2R,3R,4R,5S)-3,4-dihydroxy-5-((6-(trifluoromethyl)pyrazin-2-yl)amino)tetrahydro-2H-pyran-2-yl)-15-oxo-2,5,8,11,18-pentaoxa-14-azanonadecan-19-yl)propane-1,3-diyl)bis(oxy))bis(N-(1-((2R,3R,4R,5S)-3,4-dihydroxy-5-((6-(trifluoromethyl)pyrazin-2-yl)amino)tetrahydro-2H-pyran-2-yl)-2,5,8,11-tetraoxatridecan-13-yl)propanamide)(A87)

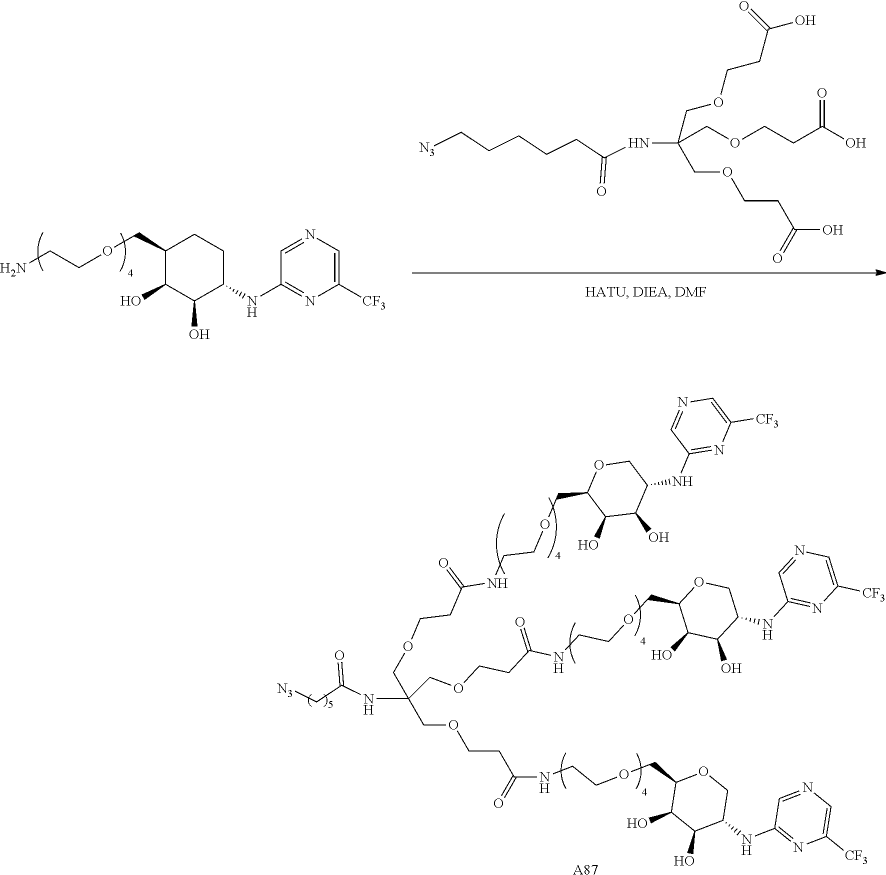

A solution of 3,3′-((2-(6-azidohexanamido)-2-((2-carboxyethoxy)methyl)propane-1,3-diyl)bis(oxy))dipropionic acid (280 mg, 0.59 mmol), HATU (893.8 mg, 2.35 mmol) and DIEA (0.58 mL, 3.53 mmol) in DMF (10 mL) was stirred at an ambient temperature for 30 min. (2R,3R,4R,5S)-2-(13-amino-2,5,8,11-tetraoxatridecyl)-5-((6-(trifluoromethyl)pyrazin-2-yl)amino)tetrahydro-2H-pyran-3,4-diol (1195.7 mg, 2.47 mmol) was added at an ambient temperature. The reaction was stirred overnight. The resulting mixture was concentrated in vacuo. The crude product was purified by Prep-HPLC(MeCN/H₂O) to give 3,3′-((2-(6-azidohexanamido)-2-(1-((2R,3R,4R,5S)-3,4-dihydroxy-5-((6-(trifluoromethyl)pyrazin-2-yl)amino)tetrahydro-2H-pyran-2-yl)-15-oxo-2,5,8,11,18-pentaoxa-14-azanonadecan-19-yl)propane-1,3-diyl)bis(oxy))bis(N-(1-((2R,3R,4R,5S)-3,4-dihydroxy-5-((6-(trifluoromethyl)pyrazin-2-yl)amino)tetrahydro-2H-pyran-2-yl)-2,5,8,11-tetraoxatridecan-13-yl)propanamide) (302.1 mg, yield: 27.4%) as a white solid. LC-MS (ESI) found: 1876 [M+H]⁺. ¹H NMR (400 MHz, CD₃OD): δ 8.12 (s, 3H), 8.00 (s, 3H), 4.34 (td, J=10.6, 5.1 Hz, 3H), 4.12 (dd, J=11.0, 5.2 Hz, 3H), 3.93 (d, J=3.0 Hz, 3H), 3.70-3.59 (m, 62H), 3.55 (t, J=5.5 Hz, 6H) 3.38 (t, J=5.5 Hz, 6H), 3.12 (t, J=10.8 Hz, 3H), 2.44 (t, J=6.1 Hz, 6H), 2.21 (t, J=7.5 Hz, 2H) 1.59 (dd, J=14.7, 7.3 Hz, 4H), 1.43-1.36 (m, 2H). ¹⁹F NMR (377 MHz, CD₃OD): δ −70.22 (s).

### Preparation of N-(((2R,3R,4R,5S)-3,4-dihydroxy-5-((6-(trifluoromethyl)pyrazin-2-yl)amino)tetrahydro-2H-pyran-2-yl)methyl)-5,6,7,8-tetrahydro-1,5-naphthyridine-2-carboxamide (A88)

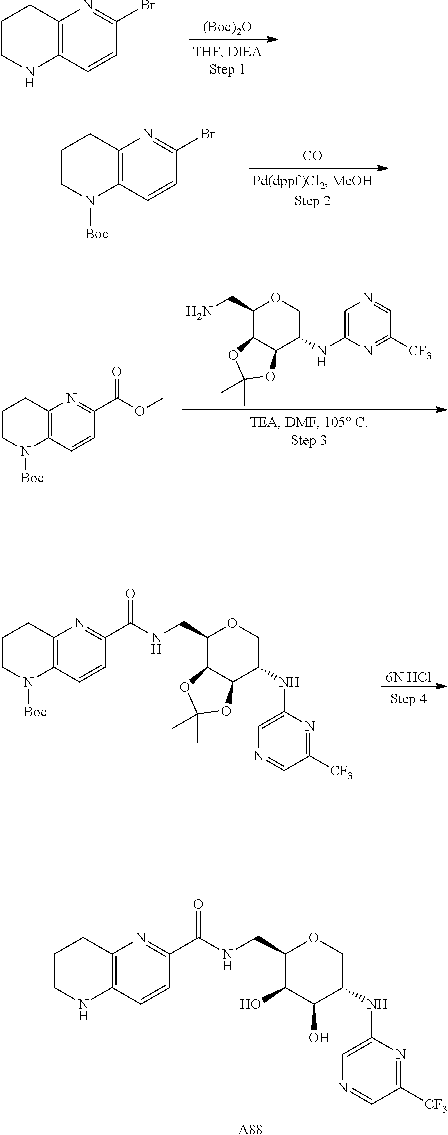

Step 1: To a solution of 6-bromo-1,2,3,4-tetrahydro-1,5-naphthyridine (1.0 g, 4.70 mmol) in THF (20.0 mL) at the room temperature was added DIEA (1.85 g, 14.1 mmol), Boc₂O (2.0 g, 9.4 mmol), the mixture was stirred at 80° C. for 4 h. The mixture was concentrated under reduced pressure to give a crude product, which was purified by column to give tert-butyl 6-bromo-3,4-dihydro-1,5-naphthyridine-1(2H)-carboxylate (1.1 g, 3.5 mmol). LC-MS (ESI) found: 313 [M+H]⁺.
Step 2: To a solution of tert-butyl 6-bromo-3,4-dihydro-1,5-naphthyridine-1(2H)-carboxylate (1.1 g, 3.5 mmol) in MeOH (20.0 mL) was added TEA (1.06 g, 10.5 mmol), Pd(dppf)C12 (256.0 mg, 0.35 mmol), The mixture was stirred at 80° C. under CO for 16 h. The mixture was filtered, concentrated under reduced pressure to give a crude product, which was purified by column chromatography to give 1-(tert-butyl) 6-methyl 3,4-dihydro-1,5-naphthyridine-1,6(2H)-dicarboxylate (584.0 mg, 2.0 mmol, 57.1%). LC-MS (ESI) found: 293 [M+H]⁺.
Step 3: To a solution of 1-(tert-butyl) 6-methyl 3,4-dihydro-1,5-naphthyridine-1,6(2H)-dicarboxylate (584.0 mg, 2.0 mmol) in toluene (20.0 mL) was added TEA (1.22 g, 6.0 mmol), N-((3aS,4R,7S,7aR)-4-(aminomethyl)-2,2-dimethyltetrahydro-4H-[1,3]dioxolo[4,5-c]pyran-7-yl) 6-(trifluoromethyl)pyrazin-2-amine (696.0 mg, 2.0 mmol), the mixture was stirred at 105° C. for 16 h. The mixture was concentrated under reduced pressure to give a crude product, which was purified by column chromatography to give tert-butyl 6-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-3,4-dihydro-1,5-naphthyridine-1(2H)-carboxylate (608.0 mg, 1.0 mmol, 50.0%). LC-MS (ESI) found: 609 [M+H]⁺.
Step 4: To a solution of tert-butyl 6-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-3,4-dihydro-1,5-naphthyridine-1(2H)-carboxylate (608.0 mg, 1.0 mmol) in THF (20.0 mL) was added HCl/H₂O (20.0 mL, 6N), the mixture was stirred at the room temperature for 16 h. The mixture was quenched by NH₃·H₂O to adjust the pH to 7.0 and concentrated under reduced pressure to give a crude product, which was purified by column chromatography to give N-(((2R,3R,4R,5S)-3,4-dihydroxy-5-((6-(trifluoromethyl)pyrazin-2-yl)amino)tetrahydro-2H-pyran-2-yl)methyl)-5,6,7,8-tetrahydro-1,5-naphthyridine-2-carboxamide (140.0 mg, 0.3 mmol, 30.0%). LC-MS (ESI) found: 469 [M+H]⁺. ¹H NMR (400 MHz, CD₃OD) δ 8.10 (s, 1H), 8.00 (s, 1H), 7.63 (d, J=8.4 Hz, 1H), 6.80 (d, J=8.4 Hz, 1H), 4.36 (td, J=10.6, 5.2 Hz, 1H), 4.14 (dd, J=11.0, 5.2 Hz, 1H), 3.88 (d, J=3.1 Hz, 1H), 3.67 (ddd, J=13.9, 11.9, 4.1 Hz, 2H), 3.62-3.56 (m, 1H), 3.51 (dd, J=13.2, 7.6 Hz, 1H), 3.33 (s, 2H), 3.12 (t, J=10.9 Hz, 1H), 2.89 (t, J=6.4 Hz, 2H), 2.06-1.92 (m, 2H). 19F NMR (377 MHz, CD₃OD) δ −70.32 (s).

### Preparation of N-(((2R,3R,4R,5S)-3,4-dihydroxy-5-((6-(trifluoromethyl)pyrazin-2-yl)amino)tetrahydro-2H-pyran-2-yl)methyl)isonicotinamide(A89)

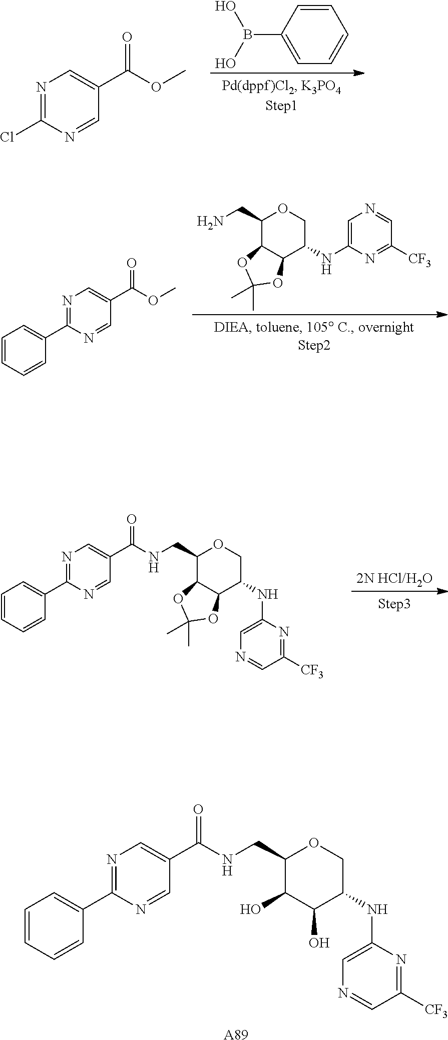

Step 1: To solution of methyl 2-chloropyrimidine-5-carboxylate (500 mg, 2.864 mmol) in dioxane-water (12 mL) was added K₂CO₃ (1200 mg, 2.864 mmol) phenylboronic acid (387 mg, 3.174 mmol) Pd(dppf)Cl₂ (423 mg, 2.864 mmol) at 88° C. The mixture was stirred at an ambient temperature overnight. The residue was purified by flash chromatography (silica gel, 0-50% PE in EA) to give methyl 2-phenylpyrimidine-5-carboxylate (412 mg, 1.92 mmol, 70.31%). LC-MS (ESI) found: 215 [M+H]⁺.
Step 2: To solution of methyl 2-phenylpyrimidine-5-carboxylate (50 mg, 0.233 mmol) in Toluene (2 mL) was added N-[(3aS,4R,7S,7aR)-4-(aminomethyl)-2,2-dimethyl-hexahydro-[1,3]dioxolo[4,5-c]pyran-7-yl]-6-(trifluoromethyl)pyrazin-2-amine (73.9 mg, 0.212 mmol) DIEA (0.035 mL, 0.212 mmol) at 105° C. for overnight. The residue was purified by flash chromatography (silica gel, 0˜10% MeOH in DCM) to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-2-phenylpyrimidine-5-carboxamide(108 mg, 0.20 mmol, 87.22%) LC-MS (ESI) found: 531 [M+H]⁺.
Step 3: To solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-2-phenylpyrimidine-5-carboxamide(30 mg 0.06 mmol) in THF (3 mL) was added 2N HCl/H₂O (6 ml) and the mixture was stirred at an ambient temperature for 1 h. The pH of the reaction mixture was adjusted to 7 and concentrated. The residue was purified by Prep-HPLC (MeCN/H₂O, 0.1% formic acid) to give N-(((2R,3R,4R,5S)-3,4-dihydroxy-5-((6-(trifluoromethyl)pyrazin-2-yl)amino)tetrahydro-2H-pyran-2-yl)methyl)-2-phenylpyrimidine-5-carboxamide (10.2 mg, yield: 36.8%). LC-MS (ESI) found: 491 [M+H]⁺. ¹H NMR (400 MHz, CD₃OD) δ 9.22 (s, 2H), 8.49 (dd, J=8.0, 1.7 Hz, 2H), 8.11 (s, 1H), 8.00 (s, 1H), 7.53 (s, 3H), 4.40-4.33 (m, 1H), 4.16 (dd, J=11.0, 5.2 Hz, 1H), 3.94 (d, J=3.0 Hz, 1H), 3.76 (dd, J=13.3, 4.4 Hz, 1H), 3.71-3.67 (m, 2H), 3.58 (dd, J=13.2, 7.6 Hz, 1H) 3.12 (d, J=10.8 Hz, 1H).

### Preparation of (2R,3R,4R,5S)-2-(piperazin-1-ylmethyl)-5-((6-(trifluoromethyl)pyrazin-2-yl)amino)tetrahydro-2H-pyran-3,4-diol (A91)

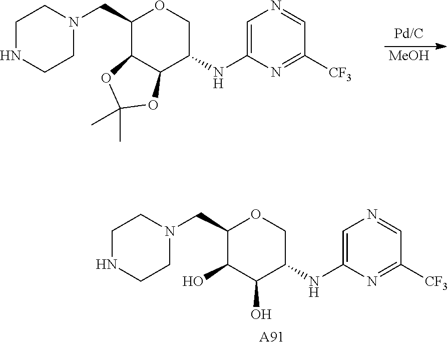

A solution of N-((3aS,4R,7S,7aR)-2,2-dimethyl-4-(piperazin-1-ylmethyl)tetrahydro-4H-[1,3]dioxolo[4,5-c]pyran-7-yl)-6-(trifluoromethyl)pyrazin-2-amine (100 mg, 0.24 mmol) in HCl (2 mL, 3N) was stirred at an ambient temperature for 2 h. The mixture was concentrated in vacuo. The residue was purified by prep-HPLC to give (2R,3R,4R,5S)-2-(piperazin-1-ylmethyl)-5-((6-(trifluoromethyl)pyrazin-2-yl)amino)tetrahydro-2H-pyran-3,4-diol (11.7 mg, 13%) as a pale yellow solid. LC-MS (ESI) found: 378 [M+H]⁺. ¹H NMR (400 MHz, CD₃OD) δ 8.12 (s, 1H), 8.00 (s, 1H), 4.38-4.27 (m, 1H), 4.13 (dd, J=10.9, 5.1 Hz, 1H), 3.87 (s, 1H), 3.70-3.57 (m, 2H) 3.28-3.15 (m, 4H), 3.11 (t, J=10.8 Hz, 1H), 2.89-2.72 (m, 5H), 2.71-2.65 (m, 1H).

### Preparation of N-(6-aminospiro[3.3]heptan-2-yl)-2-((2R,3R,4R,5S)-3,4-dihydroxy-5-((6-(trifluoromethyl)pyrazin-2-yl)amino)tetrahydro-2H-pyran-2-yl)acetamide (A92)

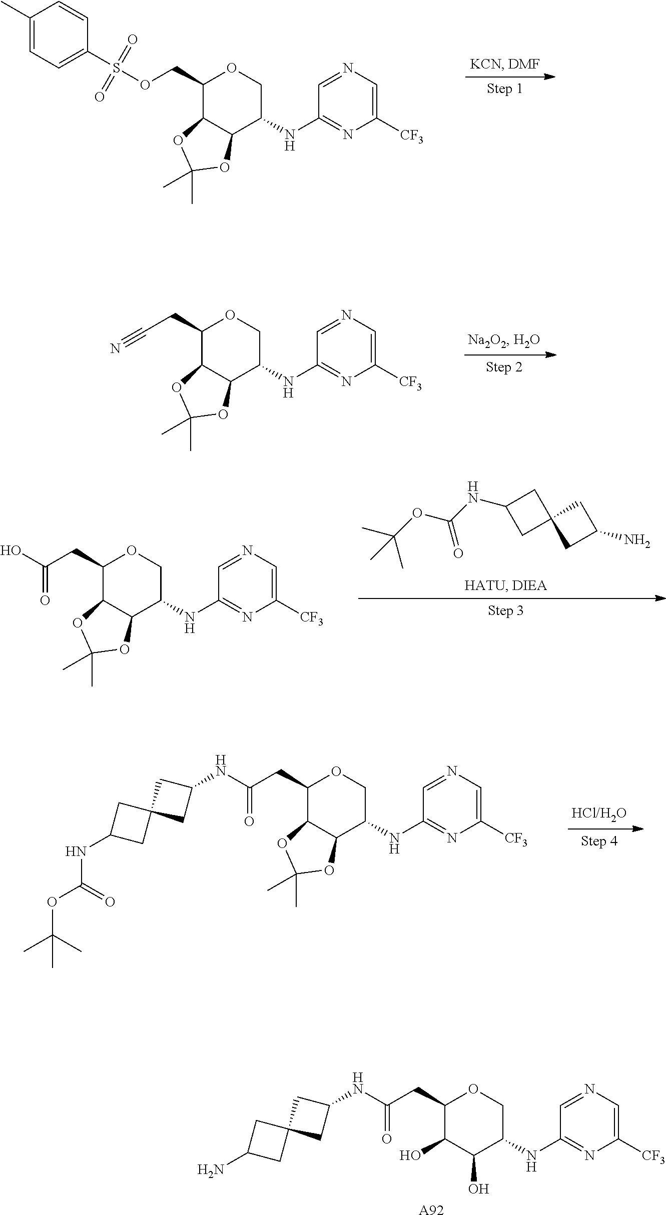

Step 1: A suspension of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (1 g, 2 mmol), KCN (0.65 g, 10 mmol) and NaI (300 mg, 2 mmol) in DMF (10 mL) was stirred at 100° C. for 48 h. The mixture was diluted with water (30 mL) and extracted with DCM (10 mL×3). The combined organic layers were washed with brine (20 mL×3), dried with anhydrous Na₂SO₄, filtered and concentrated. The residue was purified by flash chromatography reverse phase (C18, MeOH in H₂O from 5% to 60%) to give 2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetonitrile (600 mg, 60%) as a white solid. LC-MS (ESI) found: 359 [M+H]⁺. ¹H NMR (400 MHz, DMSO-d₆) δ 8.21 (s, 1H), 8.12 (s, 1H), 7.83 (d, J=7.9 Hz, 1H), 4.20-4.14 (m, 2H), 4.13-4.05 (m, 2H) 3.84 (dd, J=11.3, 4.7 Hz, 1H), 3.14 (t, J=11.0 Hz, 1H), 2.95 (dd, J=16.9, 4.7 Hz, 1H), 2.82 (dd, J=16.9, 9.0 Hz, 1H), 1.43 (s, 3H), 1.28 (s, 3H).
Step 2: A solution of 2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetonitrile (600 mg, 1.7 mmol) and Na₂O₂ (650 mg, 8.4 mmol) in H₂O (3 mL) was stirred at 60° C. overnight. The reaction mixture was cooled, quenched by saturated solution of Na₂S₂O₃ and then concentrated to dryness. The residue was purified by flash chromatography reversed phase (C18, MeOH in H₂O from 5% to 60%) to give 2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetic acid (200 mg, 31%) as a white solid. LC-MS (ESI) found: 378 [M+H]⁺.
Step 3: A solution of 2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetic acid (300 mg, 0.8 mmol) and HATU (507 mg, 1.3 mmol) in DMF (3 mL) was stirred at an ambient temperature for 2 h, then DIEA (308 mg, 2.7 mmol) and tert-butyl (6-aminospiro[3.3]heptan-2-yl)carbamate (261 mg, 1.2 mmol) were added. The mixture was stirred at an ambient temperature overnight. The mixture was concentrated under reduced pressure. The residue was purified by flash chromatography (MeOH/DCM=1:20) to give tert-butyl (6-(2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetamido)spiro[3.3]heptan-2-yl)carbamate (200 mg, 0.342 mmol, 42.95%) as a yellow solid. LC-MS (ESI) found: 586 [M+H]⁺.
Step 4: A solution of tert-butyl (6-(2-((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)acetamido) spiro[3.3]heptan-2-yl)carbamate (200 mg, 0.4 mmol) in HCl (3 mL, 3N) was stirred at an ambient temperature overnight. The pH of the mixture was adjusted to 8 by Et₃N, then concentrated. The residue was purified by flash chromatography (reverse phase, C18, MeOH in H₂O from 5% to 40%) to give N-(6-aminospiro[3.3]heptan-2-yl)-2-((2R,3R,4R,5S)-3,4-dihydroxy-5-((6-(trifluoromethyl)pyrazin-2-yl)amino)tetrahydro-2H-pyran-2-yl)acetamide (30 mg, 19%) as a white solid. LC-MS (ESI) found: 446[M+H]⁺. ¹H NMR (400 MHz, CD₃OD) δ 8.10 (s, 1H), 7.99 (s, 1H), 4.30 (td, J=10.6, 5.2 Hz, 1H), 4.18 (p, J=8.2 Hz, 1H), 4.06 (dd, J=11.0, 5.2 Hz, 1H) 3.82 (dd, J=9.1, 4.2 Hz, 1H), 3.77 (d, J=3.0 Hz, 1H), 3.70-3.59 (m, 2H), 3.08 (t, J=10.9 Hz, 1H), 2.59 (dd, J=14.7, 8.9 Hz, 1H), 2.54-2.44 (m, 2H), 2.40-2.29 (m, 3H), 2.21-2.10 (m, 2H), 2.07-1.98 (m, 2H).

### Preparation of (2R,3R,4R,5R,6S)-2-(hydroxymethyl)-6-phenyl-5-((4-(trifluoromethyl) pyrimidin-2-yl)amino)tetrahydro-2H-pyran-3,4-diol (A94)

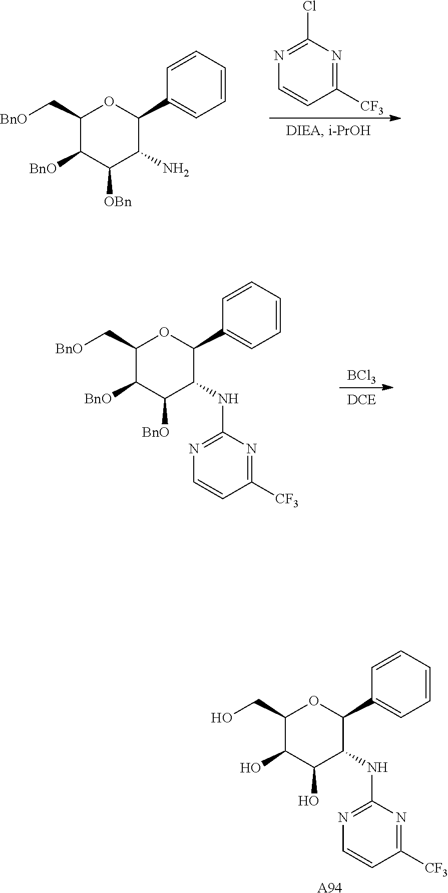

### Step 1

A solution of (2S,3S,4R,5R,6R)-4,5-bis(benzyloxy)-6-((benzyloxy)methyl)-2-phenyltetrahydro-2H-pyran-3-amine (1.00 eq), 2-chloro-4-(trifluoromethyl)pyrimidine (1.20 eq) and DIPEA (2.00 eq) in i-PrOH (5 mL) was stirred at 100° C. overnight. The mixture was concentrated and purified by reverse phase (5-30% MeCN in H₂O, 0.3% TFA) to afford N-((2S,3S,4R,5R,6R)-4,5-bis(benzyloxy)-6-((benzyloxy)methyl)-2-phenyltetrahydro-2H-pyran-3-yl)-4-(trifluoromethyl)pyrimidin-2-amine as a colorless oil. Chemical Formula: C₃₈H₃₆F₃N₃O₄, LCMS found: [M+H]⁺=656.

### Step 2

To a solution of N-((2S,3S,4R,5R,6R)-4,5-bis(benzyloxy)-6-((benzyloxy)methyl)-2-phenyltetrahydro-2H-pyran-3-yl)-4-(trifluoromethyl)pyrimidin-2-amine (1.00 eq) in DCE (5 mL) was added BCl₃ (5.00 eq, 1N) at −40° C. The mixture was stirred at an ambient temperature for 2 h, then quenched with MeOH. The mixture was concentrated and then purified by reverse phase column (5-20% MeCN in H₂O, 0.1% TFA) to afford (2R,3R,4R,5R,6S)-2-(hydroxymethyl)-6-phenyl-5-((4-(trifluoromethyl)pyrimidin-2-yl)amino)tetrahydro-2H-pyran-3,4-diol as a white solid. Chemical Formula: C₁₇H₁₈F₃N₃O₄, LCMS found: [M+H]⁺=386. ¹H NMR (400 MHz, CD₃OD): δ 8.46 (d, J=4.9 Hz, 1H), 7.54 (d, J=7.6 Hz, 2H), 7.33 (t, J=7.6 Hz, 2H), 7.24 (t, J=7.1 Hz, 1H), 6.85 (d, J=4.6 Hz, 1H), 5.02 (d, J=4.1 Hz, 1H), 4.04 (dd, J=11.8, 7.5 Hz, 1H) 3.93 (s, 1H), 3.78 (dd, J=11.8, 3.6 Hz, 2H), 3.71-3.62 (m, 1H).

### Preparation of N-((2R,3R,4R,5R,6R)-4,5-dihydroxy-6-(hydroxymethyl)-2-phenyltetrahydro-2H-pyran-3-yl)acetamide (A95)

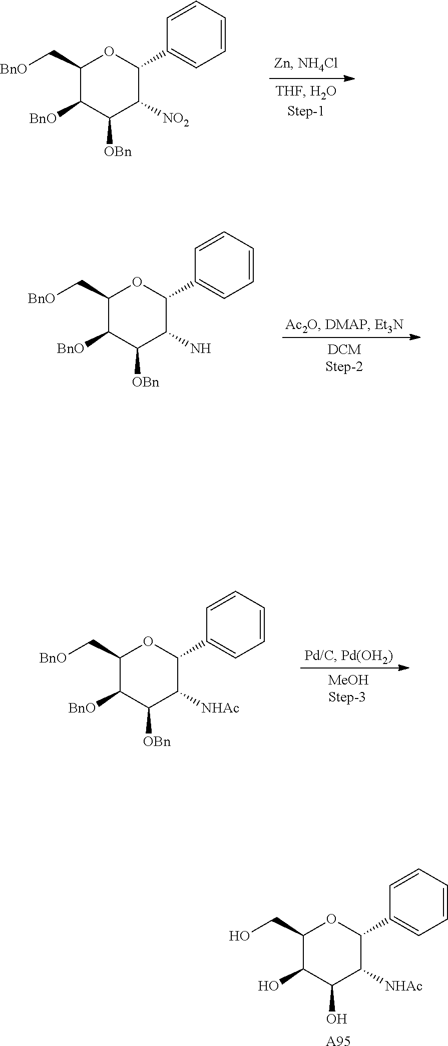

A solution of (2R,3R,4R,5S,6R)-3,4-bis(benzyloxy)-2-((benzyloxy)methyl)-5-nitro-6-phenyltetrahydro-2H-pyran (1.00 eq), Zn (10.0 eq) and NH₄Cl (10.0 eq) in THF and H₂O was stirred at 70° C. overnight. The mixture was filtered through a Celite pad, and the filtrate was concentrated to afford (2R,3S,4R,5R,6R)-4,5-bis(benzyloxy)-6-((benzyloxy)methyl)-2-phenyltetrahydro-2H-pyran-3-amine as a colorless oil. Chemical Formula: C₃₃H₃₅NO₄, LCMS found: [M+H]⁺=510.

### Step 2

To a solution of (2R,3S,4R,5R,6R)-4,5-bis(benzyloxy)-6-((benzyloxy)methyl)-2-phenyltetrahydro-2H-pyran-3-amine (1.00 eq) in DCM was added DMAP (0.1 eq) and Ac₂O (2.00 eq) at 0° C. The mixture was stirred at 25° C. overnight. Then the mixture was concentrated and purified by silica gel chromatography to afford N-((2R,3S,4R,5R,6R)-4,5-bis(benzyloxy)-6-((benzyloxy)methyl)-2-phenyltetrahydro-2H-pyran-3-yl)acetamide as a white solid. Chemical Formula: C₁₆H₃₇NO₅, LCMS found: [M+H]⁺=552.

### Step 3

To a solution of N-((2R,3S,4R,5R,6R)-4,5-bis(benzyloxy)-6-((benzyloxy)methyl)-2-phenyltetrahydro-2H-pyran-3-yl)acetamide (1.00 eq) in MeOH were added Pd/C (10% purity) and Pd(OH)₂ (10% purity) at 25° C. The mixture was stirred at 25° C. under a H2 balloon overnight. The mixture was filtered through a Celite pad, and the filtrate was concentrated to afford N-((2R,3R,4R,5R,6R)-4,5-dihydroxy-6-(hydroxymethyl)-2-phenyltetrahydro-2H-pyran-3-yl)acetamide as a white solid. Chemical Formula: C₁₄H₁₉NO₅, LCMS found: [M+H]⁺=282. ¹H NMR (400 MHz, CD₃OD): δ 7.38 (t, J=12.2 Hz, 2H), 7.27 (dd, J=19.5, 11.8 Hz, 2H), 7.21 (t, J 20=7.3 Hz, 1H), 5.31 (d, J=2.5 Hz, 1H), 4.32 (dt, J=11.9, 6.0 Hz, 1H), 4.23 (ddd, J=17.4, 10.9, 7.0 Hz, 1H), 4.13 (dd, J=7.3, 4.8 Hz, 2H), 3.92 (s, 1H), 3.81 (dt, J=8.0, 3.9 Hz, 1H), 1.74 (s, 3H).

### Preparation of (2R,3R,4R,5R,6R)-2-(hydroxymethyl)-6-phenyl-5-((4-(trifluoromethyl) pyrimidin-2-yl)amino)tetrahydro-2H-pyran-3,4-diol (A96)

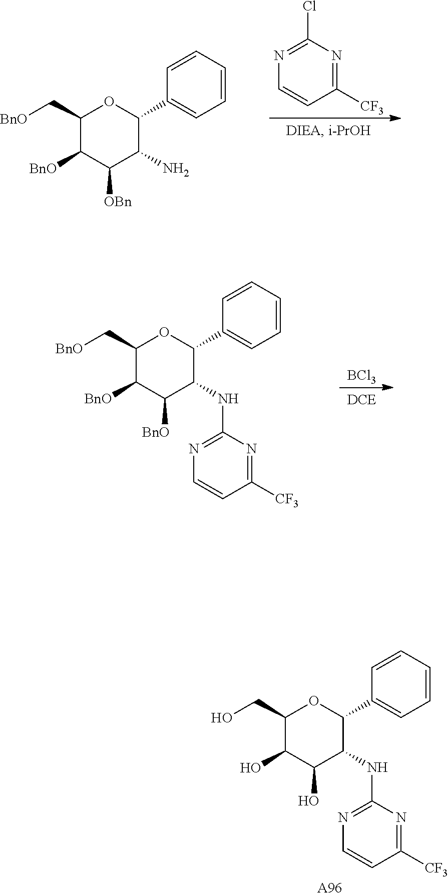

### Step 1

A solution of (2R,3S,4R,5R,6R)-4,5-bis(benzyloxy)-6-((benzyloxy)methyl)-2-phenyltetrahydro-2H-pyran-3-amine (1.00 eq), 2-chloro-4-(trifluoromethyl)pyrimidine (1.50 eq) and DIPEA (2.00 eq) in i-PrOH was stirred at 100° C. overnight. The mixture was concentrated and then purified by reverse phase (5-30% MeCN in water, 0.3% TFA) to afford N-((2R,3S,4R,5R,6R)-4,5-bis(benzyloxy)-6-((benzyloxy)methyl)-2-phenyltetrahydro-2H-pyran-3-yl)-4-(trifluoromethyl)pyrimidin-2-amine as a colorless oil. Chemical Formula: C₃₈H₃₆F₃N₃O₄, LCMS found: [M+H]⁺=656.

### Step 2

To a solution of N-((2R,3S,4R,5R,6R)-4,5-bis(benzyloxy)-6-((benzyloxy)methyl)-2-phenyltetrahydro-2H-pyran-3-yl)-4-(trifluoromethyl)pyrimidin-2-amine (1.00 eq) in DCE was added BCl₃ (5.00 eq) at −40° C. The mixture was stirred at 25° C. for 2 h, then quenched with MeOH. The mixture was concentrated and then purified by reverse phase (5-25% MeCN in water, 0.1% TFA) to afford (2R,3R,4R,5R,6R)-2-(hydroxymethyl)-6-phenyl-5-((4-(trifluoromethyl)pyrimidin-2-yl)amino)tetrahydro-2H-pyran-3,4-diol as a white solid. Chemical Formula: C₁₇H₁₈F₃N₃O₄, LCMS found: [M+H]⁺=386. ¹H NMR (400 MHz, CD₃OD): δ 8.38 (s, 1H), 7.44 (d, J=7.6 Hz, 2H), 7.23 (t, J=7.2 Hz, 2H), 7.13 (t, J=6.9 Hz, 1H), 6.79 (d, J=4.9 Hz, 1H), 5.44 (d, J=2.3 Hz, 1H), 4.56 (dd, J=4.9, 3.0 Hz, 1H), 4.28-4.18 (m, 2H), 4.16-4.06 (m, 2H), 3.84 (dd, J=12.3, 3.0 Hz, 1H).

### Preparation of 4-(4-((4-(((2R,3R,4R,5S)-3,4-dihydroxy-5-((2-(trifluoromethyl)pyrimidin-4-yl)amino)tetrahydro-2H-pyran-2-yl)methyl)piperazin-1-yl)methyl)piperidin-1-yl)benzoic acid (A97)

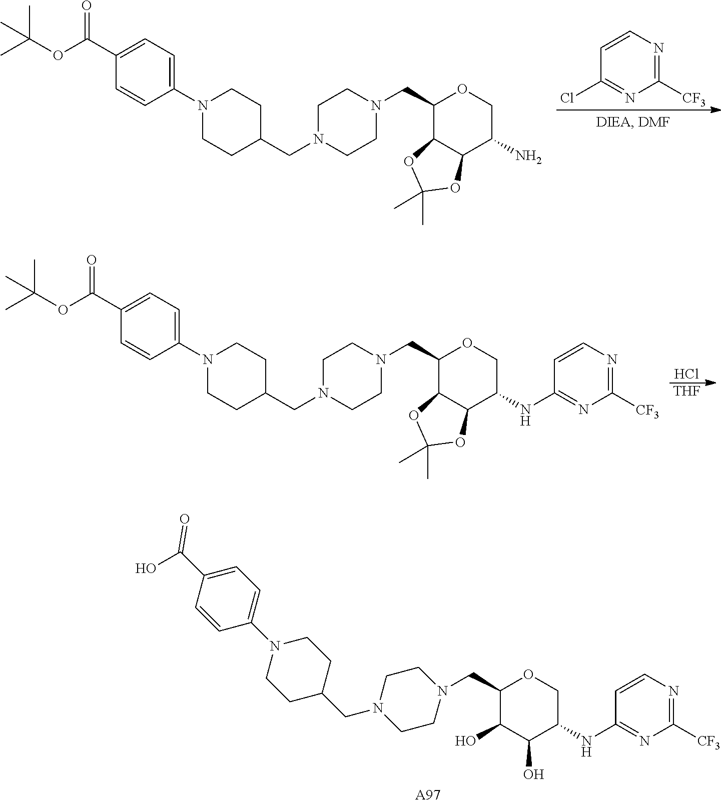

To a solution of tert-butyl 4-(4-((4-(((3aS,4R,7S,7aR)-7-amino-2,2-dimethyltetrahydro-4H-[1,3]dioxolo[4,5-c]pyran-4-yl)methyl)piperazin-1-yl)methyl)piperidin-1-yl)benzoate (1.00 eq) and 4-chloro-2-(trifluoromethyl)pyrimidine (1.30 eq) in DMF was added DIEA (3.00 eq) at 25° C., the mixture was stirred at 100° C. for 16 h. The resulting mixture was diluted with ethyl acetate, washed with H₂O and brine, the organic layer was separated, dried over Na₂SO₄ and concentrated in vacuo. The crude product was purified by flash chromatography (silica gel, 0˜10% MeOH in DCM) to give tert-butyl 4-(4-((4-(((3aS,4R,7S,7aR)-2,2-dimethyl-7-((2-(trifluoromethyl)pyrimidin-4-yl)amino)tetrahydro-4H-[1,3]dioxolo[4,5-c]pyran-4-yl)methyl)piperazin-1-yl)methyl)piperidin-1-yl)benzoate as a brown solid. Chemical Formula: C₃₅H₄₉F₃N₆O₅, LCMS found: [M+H]⁺=691.

### Step 2

A solution of tert-butyl 4-(4-((4-(((3aS,4R,7S,7aR)-2,2-dimethyl-7-((2-(trifluoromethyl)pyrimidin-4-yl)amino)tetrahydro-4H-[1,3]dioxolo[4,5-c]pyran-4-yl)methyl)piperazin-1-yl)methyl)piperidin-1-yl)benzoate (1.00 eq) in THE was added HCl (2 N). The reaction was stirred at 50° C. for 16 h. The resulting mixture was concentrated in vacuo. The pH of the aqueous phase was adjusted to 7 by saturated solution of sodium bicarbonate, then concentrated under reduced pressure, and the crude product was purified by reverse phase chromatography (C18, 0-50% MeCN in water, 0.3% TFA) to give 4-(4-((4-(((2R,3R,4R,5S)-3,4-dihydroxy-5-((2-(trifluoromethyl)pyrimidin-4-yl)amino)tetrahydro-2H-pyran-2-yl)methyl)piperazin-1-yl)methyl)piperidin-1-yl)benzoic acid as a white solid. Chemical Formula: C₂₈H₃₇F₃N₆O₅. ¹H NMR (400 MHz, CD₃OD): δ 8.12 (s, 1H), 7.84 (d, J=8.9 Hz, 2H), 6.66 (s, 1H), 6.54 (d, J=9.0 Hz, 2H), 4.47 (s, 1H), 4.13 (s, 1H), 3.86 (d, J=2.9 Hz, 1H), 3.66 (dd, J=15.4, 5.0 Hz, 2H), 3.55 (dd, J=9.5, 7.4 Hz, 1H), 3.49-3.43 (m, 1H), 3.35 (dd, J=12.9, 5.6 Hz, 1H), 3.13 (s, 1H), 2.90 (ddd, J=16.5, 15.5, 5.7 Hz, 13H), 2.34 (dd, J=15.7, 7.6 Hz, 1H), 2.24 (dtd, J=9.6, 6.6, 3.1 Hz, 1H), 1.82-1.70 (m, 3H). ¹⁹F NMR (377 MHz, CD₃OD): δ −72.90 (s), -76.91 (s). LCMS found: [M+H]⁺=595.

### Preparation of (2R,3R,4R,5S)-2-(aminomethyl)-5-((6-(trifluoromethyl)pyridin-2-yl)amino) tetrahydro-2H-pyran-3,4-diol (A98)

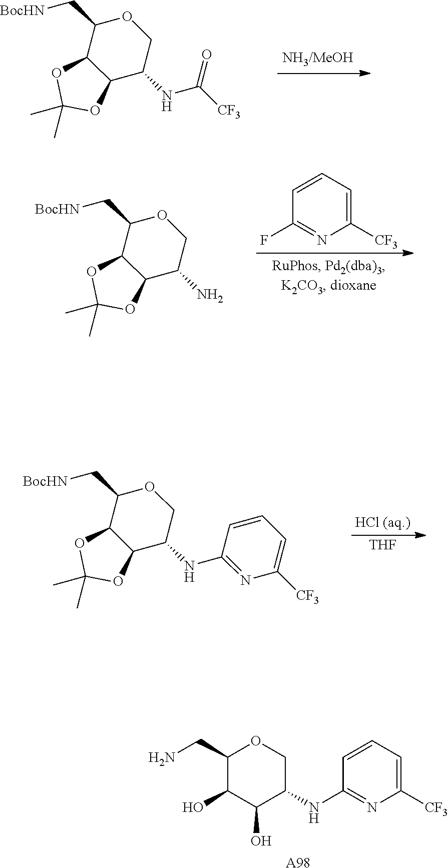

### Step 1

The solution of tert-butyl (((3aS,4R,7S,7aR)-2,2-dimethyl-7-(2,2,2-trifluoroacetamido)tetrahydro-4H-[1,3]dioxolo[4,5-c]pyran-4-yl)methyl)carbamate (1.00 eq) in NH₃/MeOH (7 N) was stirred at 80° C. for 16 hours in sealed tube. Then the reaction was concentrated and purified by silica gel chromatography to afford tert-butyl (((3aS,4R,7S,7aR)-7-amino-2,2-dimethyltetrahydro-4H-[1,3]dioxolo[4,5-c]pyran-4-yl)methyl)carbamate as a colorless oil. Chemical Formula: C₂₁H₄₁Cl₂NO₈, LCMS found: [M+H]⁺=303.

### Step 2

To a solution of tert-butyl (((3aS,4R,7S,7aR)-7-amino-2,2-dimethyltetrahydro-4H-[1,3]dioxolo[4,5-c]pyran-4-yl)methyl)carbamate (1.00 eq) and 2-fluoro-6-(trifluoromethyl)pyridine (1.20 eq) in dioxane was added K₂CO₃ (1.50 eq), RuPhos (0.10 eq) and Pd₂(dba)₃(0.10 eq). The mixture was stirred at 110° C. for 3 hours under nitrogen atmosphere. Then the reaction was quenched with brine and extracted with ethyl acetate. The organic layer was concentrated, the residue was purified by silica gel chromatography to afford tert-butyl (((3aS,4R,7S,7aR)-2,2-dimethyl-7-((6-(trifluoromethyl)pyridin-2-yl)amino)tetrahydro-4H-[1,3]dioxolo[4,5-c]pyran-4-yl)methyl)carbamate as a colorless oil. Chemical Formula: C₂₁H₄₁N₇O₈, LCMS found: [M+H]⁺=466.

### Step 3

To a solution of tert-butyl (((3aS,4R,7S,7aR)-2,2-dimethyl-7-((6-(trifluoromethyl)pyridin-2-yl)amino)tetrahydro-4H-[1,3]dioxolo[4,5-c]pyran-4-yl)methyl)carbamate (1.00 eq) in THE was added HCl (2N) at 25° C. The mixture was stirred at 25° C. overnight. Then the mixture was concentrated to afford (2R,3R,4R,5S)-2-(aminomethyl)-5-((6-(trifluoromethyl)pyridin-2-yl)amino)tetrahydro-2H-pyran-3,4-diol as a colorless oil. Chemical Formula: C₁₆H₃₃N₇O₆, LCMS found: [M+H]⁺=308. ¹H NMR (400 MHz, DMSO) δ 7.69-7.51 (m, 1H), 7.00 (dd, J=8.0, 2.8 Hz, 1H), 6.97-6.76 (m, 2H), 4.95 (d, J=6.0 Hz, 1H), 4.25 (dt, J=15.6, 10.6 Hz, 1H), 3.95 (dd, J=10.8, 5.0 Hz, 1H), 3.78 (d, J=2.9 Hz, 1H), 3.74-3.66 (m, 1H), 3.56 (dd, J=8.1, 3.8 Hz, 1H) 3.07 (t, J=10.7 Hz, 1H), 2.97 (dd, J=12.3, 6.1 Hz, 1H).

### Preparation of (2R,3R,4R,5S)-2-(aminomethyl)-5-((3-fluoro-6-(trifluoromethyl)pyridin-2-yl)amino)tetrahydro-2H-pyran-3,4-diol (A99)

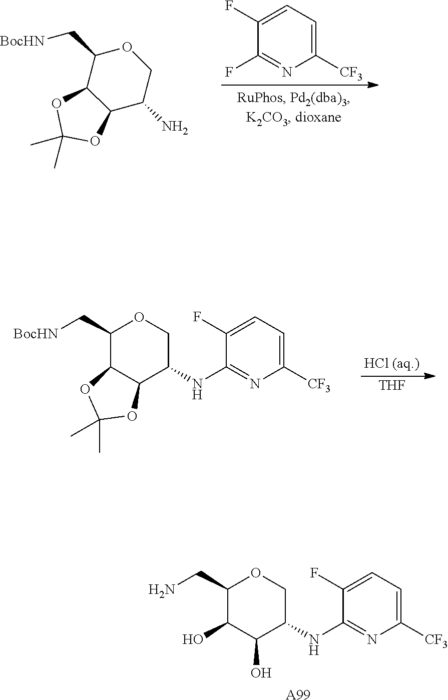

### Step 1

To a solution of tert-butyl (((3aS,4R,7S,7aR)-7-amino-2,2-dimethyltetrahydro-4H-[1,3]dioxolo[4,5-c]pyran-4-yl)methyl)carbamate (1.00 eq) and 2-bromo-3-fluoro-6-(trifluoromethyl)pyridine (1.20 eq) in dioxane was added K₂CO₃ (1.50 eq), RuPhos (0.10 eq) and Pd₂(dba)₃ (0.10 eq), the mixture was stirred at 110° C. for 3 hours under nitrogen atmosphere. Then the reaction was quenched with brine and extracted with ethyl acetate. The organic layer was concentrated and the residue was purified by silica gel chromatography to afford tert-butyl (((3aS,4R,7S,7aR)-7-((3-fluoro-6-(trifluoromethyl)pyridin-2-yl)amino)-2,2-dimethyltetrahydro-4H-[1,3]dioxolo[4,5-c]pyran-4-yl)methyl)carbamate as a colorless oil. Chemical Formula: C₂₁H₄₁N₇O₈, LCMS found: [M+H]⁺=466.

### Step 2

To a solution of tert-butyl (((3aS,4R,7S,7aR)-7-((3-fluoro-6-(trifluoromethyl)pyridin-2-yl)amino) 2,2-dimethyltetrahydro-4H-[1,3]dioxolo[4,5-c]pyran-4-yl)methyl)carbamate (1.00 eq) in THE was added HCl (2N) at 25° C. The mixture was stirred at 25° C. overnight. Then the mixture was concentrated to afford (2R,3R,4R,5S)-2-(aminomethyl)-5-((3-fluoro-6-(trifluoromethyl)pyridin-2-yl)amino)tetrahydro-2H-pyran-3,4-diol as a colorless oil. Chemical Formula: C₁₆H₃₃N₇O₆, ¹H NMR (400 MHz, DMSO) δ 7.55 (dd, J=10.9, 8.0 Hz, 1H), 7.00 (dd, J=8.0, 2.8 Hz, 1H), 6.94 (d, J=6.6 Hz, 1H), 4.95 (d, J=6.0 Hz, 1H), 4.25 (dt, J=15.6, 10.6 Hz, 1H), 3.95 (dd, J=10.8, 5.0 Hz, 1H), 3.78 (d, J=2.9 Hz, 1H), 3.74-3.66 (m, 1H), 3.56 (dd, J=8.1, 3.8 Hz, 1H), 3.07 (t, J=10.7 Hz, 1H), 2.97 (dd, J=12.3, 6.1 Hz, 1H). LCMS found: [M+H]⁺=326.

### Preparation of 4-(4-((4-(((2R,3R,4R,5S)-3,4-dihydroxy-5-((6-(trifluoromethyl)pyridin-2-yl)amino)tetrahydro-2H-pyran-2-yl)methyl)piperazin-1-yl)methyl)piperidin-1-yl)benzoic acid (A100)

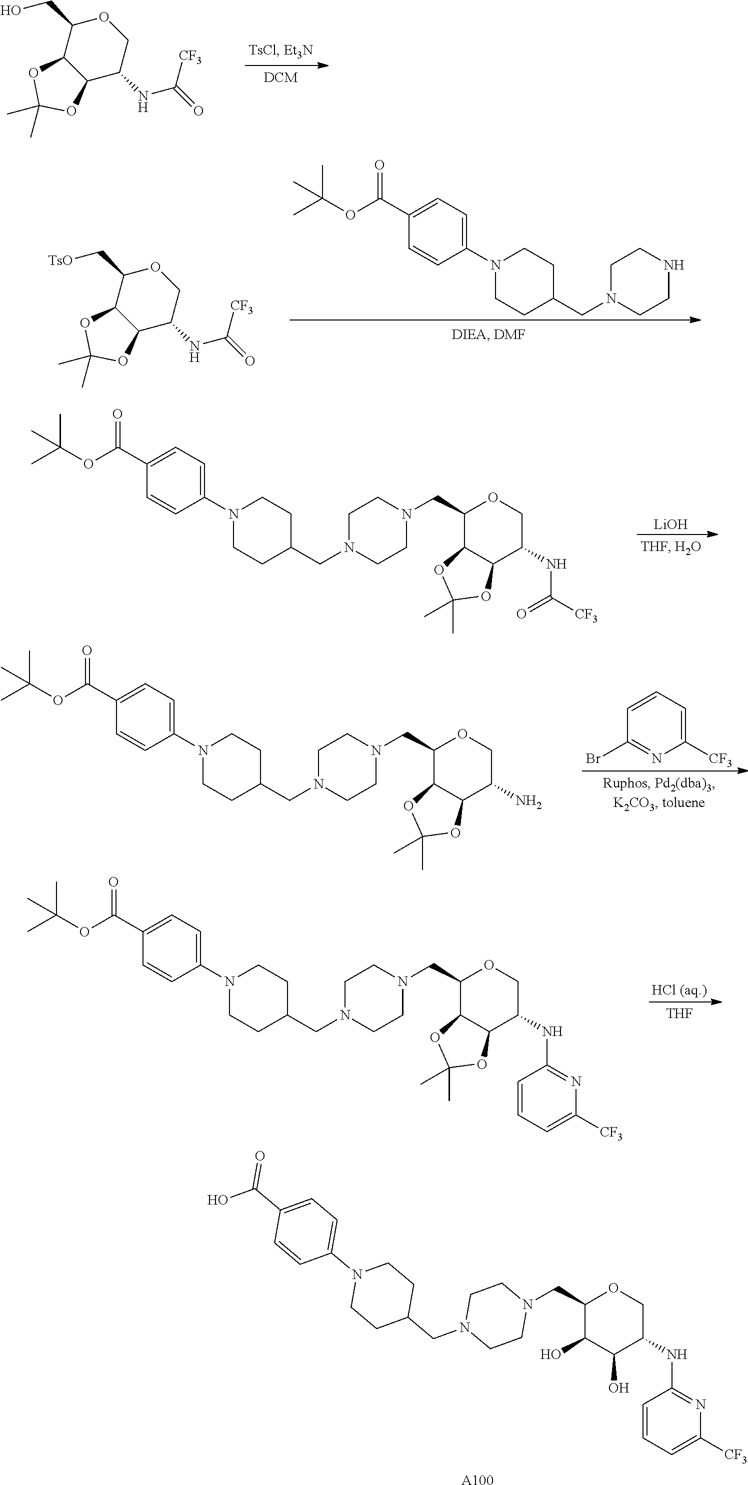

### Step 1

To a solution of 2,2,2-trifluoro-N-((3aR,4R,7S,7aR)-4-(hydroxymethyl)-2,2-dimethyltetrahydro-4H-[1,3]dioxolo[4,5-c]pyran-7-yl)acetamide (1.00 eq) and Et₃N (3.00 eq) in DCM was added TsCl (1.50 eq) at 0° C. under N₂. The reaction was stirred at 25° C. overnight. The resulting mixture was diluted with DCM, washed with H₂O and brine, dried over Na₂SO₄. The organic layer was separated and concentrated in vacuo. The crude product was purified by flash chromatography (silica gel, 0-100% ethyl acetate in petroleum ether) to give ((3aR,4R,7S,7aR)-2,2-dimethyl-7-(2,2,2-trifluoroacetamido)tetrahydro-4H-[1,3]dioxolo[4,5-c]pyran-4-yl)methyl 4-methylbenzenesulfonate as a brown solid. LC-MS (ESI) found: 454 [M+H]⁺.

### Step 2

A solution of ((3aR,4R,7S,7aR)-2,2-dimethyl-7-(2,2,2-trifluoroacetamido)tetrahydro-4H-[1,3]dioxolo[4,5-c]pyran-4-yl)methyl 4-methylbenzenesulfonate (1.00 eq) and tert-butyl 4-(4-(piperazin-1-ylmethyl)piperidin-1-yl)benzoate (1.00 eq) in DMF was added DIEA (3.00 eq), the mixture was stirred at 100° C. overnight. The resulting mixture was diluted with ethyl acetate, washed with H₂O and brine, dried over Na₂SO₄. The organic layer was separated and concentrated in vacuo. The crude product was purified by flash chromatography (silica gel, 0˜10% MeOH in DCM) to give tert-butyl 4-(4-((4-(((3aS,4R,7S,7aR)-2,2-dimethyl-7-(2,2,2-trifluoroacetamido)tetrahydro-4H-[1,3]dioxolo[4,5-c]pyran-4-yl)methyl)piperazin-1-yl)methyl)piperidin-1-yl)benzoate as a brown solid. LC-MS (ESI) found: 641 [M+H]⁺.

### Step 3

A solution of tert-butyl 4-(4-((4-(((3aS,4R,7S,7aR)-2,2-dimethyl-7-(2,2,2-trifluoroacetamido)tetrahydro-4H-[1,3]dioxolo[4,5-c]pyran-4-yl)methyl)piperazin-1-yl)methyl)piperidin-1-yl)benzoate (1.00 eq) in THE and H₂O was added LiOH (2.00 eq) at 0° C. The reaction was stirred at 25° C. for 2 h. The pH of the mixture was adjusted to 5 with HCl (2N). The resulting mixture was filtered and concentrated in vacuo. The crude product was purified by prep-HPLC to give tert-butyl 4-(4-((4-(((3aS,4R,7S,7aR)-7-amino-2,2-dimethyltetrahydro-4H-[1,3]dioxolo[4,5-c]pyran-4-yl)methyl)piperazin-1-yl)methyl)piperidin-1-yl)benzoate as a white solid. LC-MS (ESI) found: 545 [M+H]⁺.

### Step 4

A solution of tert-butyl 4-(4-((4-(((3aS,4R,7S,7aR)-7-amino-2,2-dimethyltetrahydro-4H-[1,3]dioxolo[4,5-c]pyran-4-yl)methyl)piperazin-1-yl)methyl)piperidin-1-yl)benzoate (1.00 eq), 2-bromo-6-(trifluoromethyl)pyridine (1.00 eq), Pd₂(dba)₃ (0.10 eq), RuPhos (0.10 eq) and Cs₂CO₃ (2.50 eq) in toluene was stirred at 100° C. overnight under nitrogen atmosphere. The resulting mixture was concentrated in vacuo. The crude product was purified by flash chromatography (silica gel, 0˜10% MeOH in DCM) to give tert-butyl 4-(4-((4-(((3aS,4R,7S,7aR)-2,2-dimethyl-7-((6-(trifluoromethyl)pyridin-2-yl)amino)tetrahydro-4H-[1,3]dioxolo[4,5-c]pyran-4-yl)methyl)piperazin-1-yl)methyl)piperidin-1-yl)benzoate as a brown solid. LC-MS (ESI) found: 690 [M+H]⁺.

### Step 5

A solution of tert-butyl 4-(4-((4-(((3aS,4R,7S,7aR)-2,2-dimethyl-7-((6-(trifluoromethyl)pyridin-2-yl)amino)tetrahydro-4H-[1,3]dioxolo[4,5-c]pyran-4-yl)methyl)piperazin-1-yl)methyl)piperidin-1-yl)benzoate (1.00 eq) in THF was added HCl (9.00 eq, 1N). The reaction was stirred at 50° C. overnight. The resulting mixture was filtered and concentrated in vacuo. The pH of the mixture was adjusted to 7 with saturated solution of sodium bicarbonate, then concentrated under reduced pressure. The crude product was purified by prep-HPLC to give 4-(4-((4-(((2R,3R,4R,5S)-3,4-dihydroxy-5-((6-(trifluoromethyl)pyridin-2-yl)amino)tetrahydro-2H-pyran-2-yl)methyl)piperazin-1-yl)methyl)piperidin-1-yl)benzoic acid as a white solid. LC-MS (ESI) found: 594 [M+H]⁺. ¹H NMR (400 MHz, CD₃OD): δ 8.42 (s, 1H), 7.84 (d, J=8.9 Hz, 2H) 7.53 (t, J=7.9 Hz, 1H), 6.89 (d, J=7.2 Hz, 1H), 6.73 (d, J=8.5 Hz, 1H), 6.54 (d, J=8.9 Hz, 2H), 4.32-4.26 (m, 1H), 4.22 (dd, J=10.8, 5.2 Hz, 1H), 3.86 (d, J=2.7 Hz, 1H), 3.71 (dd, J=8.2, 3.1 Hz, 1H), 3.64 (dd, J=10.4, 3.2 Hz, 1H), 3.55 (dd, J=9.4, 7.5 Hz, 1H), 3.49-3.43 (m, 1H), 3.38-3.32 (m, 1H), 3.05 (ddd, J=25.1, 16.5, 9.9 Hz, 11H), 2.91-2.86 (m, 3H), 2.35 (dt, J=14.8, 7.4 Hz, 1H), 2.28-2.19 (m, 1H), 1.77 (ddd, J=21.0, 13.6, 8.3 Hz, 3H). ¹⁹F NMR (377 MHz, CD₃OD): δ −70.27 (s).

### Preparation of 4-(4-((4-(((2R,3R,4R,5S)-5-((3-fluoro-6-(trifluoromethyl)pyridin-2-yl)amino)-3,4-dihydroxytetrahydro-2H-pyran-2-yl)methyl)piperazin-1-yl)methyl)piperidin-1-yl)benzoic acid (A101)

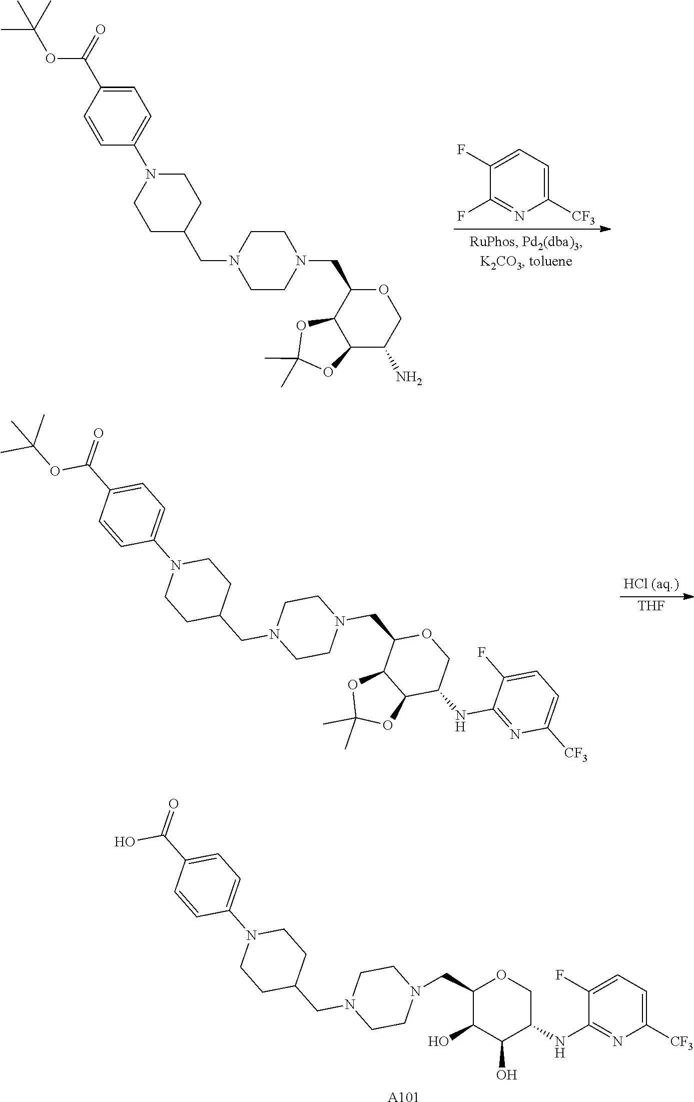 [SMILES: CC(C)(C)OC(=O)c1ccc(N2CCC(CN3CCN(C[C@H]4OC[C@H](N)[C@H]5OC(C)(C)O[C@H]54)CC3)CC2)cc1 ; Fc1ccc(C(F)(F)F)nc1F ; CC(C)(C)OC(=O)c1ccc(N2CCC(CN3CCN(C[C@H]4OC[C@H](Nc5nc(C(F)(F)F)ccc5F)[C@H]5OC(C)(C)O[C@H]54)CC3)CC2)cc1 ; "THF" ; O=C(O)c1ccc(N2CCC(CN3CCN(C[C@H]4OC[C@H](Nc5nc(C(F)(F)F)ccc5F)[C@@H](O)[C@H]4O)CC3)CC2)cc1 ; "A101"]

### Step 1

To a solution of tert-butyl 4-(4-((4-(((3aS,4R,7S,7aR)-7-amino-2,2-dimethyltetrahydro-4H-[1,3]dioxolo[4,5-c]pyran-4-yl)methyl)piperazin-1-yl)methyl)piperidin-1-yl)benzoate (1.00 eq), 2-bromo-3-fluoro-6-(trifluoromethyl)pyridine (1.00 eq), Pd₂(dba)₃ (0.10 eq), RuPhos (0.10 eq) and Cs₂CO₃ (3.00 eq) in toluene was stirred at 100° C. overnight under nitrogen atmosphere. The resulting mixture was concentrated in vacuo. The crude product was purified by flash chromatography (silica gel, 0˜10% MeOH in DCM) to give tert-butyl 4-(4-((4-(((3aS,4R,7S,7aR) 7-((3-fluoro-6-(trifluoromethyl)pyridin-2-yl)amino)-2,2-dimethyltetrahydro-4H-[1,3]dioxolo[4,5-c]pyran-4-yl)methyl)piperazin-1-yl)methyl)piperidin-1-yl)benzoate as a brown solid. LC-MS (ESI) found: 708 [M+H]⁺.

### Step 2

A solution of tert-butyl 4-(4-((4-(((3aS,4R,7S,7aR)-7-((3-fluoro-6-(trifluoromethyl)pyridin-2-yl)amino)-2,2-dimethyltetrahydro-4H-[1,3]dioxolo[4,5-c]pyran-4-yl)methyl)piperazin-1-yl)methyl)piperidin-1-yl)benzoate (1.00 eq) in THF was added HCl (9.00 eq, 2N). The reaction was stirred at 50° C. overnight. The resulting mixture was filtered and concentrated in vacuo. The pH of the mixture was adjusted to 7 with saturated solution of sodium bicarbonate, then concentrated under reduced pressure. The crude product was purified by prep-HPLC to give 4-(4-((4-(((2R,3R,4R,5S)-5-((3-fluoro-6-(trifluoromethyl)pyridin-2-yl)amino)-3,4-dihydroxytetrahydro-2H-pyran-2-yl)methyl)piperazin-1-yl)methyl)piperidin-1-yl)benzoic acid as a white solid. LC-MS (ESI) found: 612 [M+H]⁺. ¹H NMR (400 MHz, CD₃OD): δ 7.83 (d, J=8.9 Hz, 2H), 7.36 (dd, J=10.6, 8.1 Hz, 1H), 6.95 (dd, J=8.0, 2.8 Hz, 1H), 6.53 (d, J=9.0 Hz, 2H) 4.48-4.42 (m, 1H), 4.19 (dd, J=10.9, 5.2 Hz, 1H), 3.88 (d, J=2.9 Hz, 1H), 3.76 (dd, J=10.6, 3.2 Hz, 1H), 3.71 (dd, J=8.0, 3.0 Hz, 1H), 3.54 (dd, J=9.4, 7.5 Hz, 1H), 3.48-3.42 (m, 1H) 3.35 (d, J=8.9 Hz, 1H), 3.16 (d, J=10.8 Hz, 1H), 3.05-2.80 (m, 13H), 2.34 (dt, J=15.2, 7.5 Hz, 1H), 2.23 (dtd, J=9.6, 6.6, 2.9 Hz, 1H), 1.84-1.68 (m, 3H). ¹⁹F NMR (377 MHz, CD₃OD): δ −69.34 (s), −76.88 (s), −137.28 (s).

### Preparation of 1-((3S,4R,5S)-3,4-dihydroxy-5-((6-(trifluoromethyl)pyrazin-2-yl)amino)piperidin-1-yl)ethan-1-one (A102)

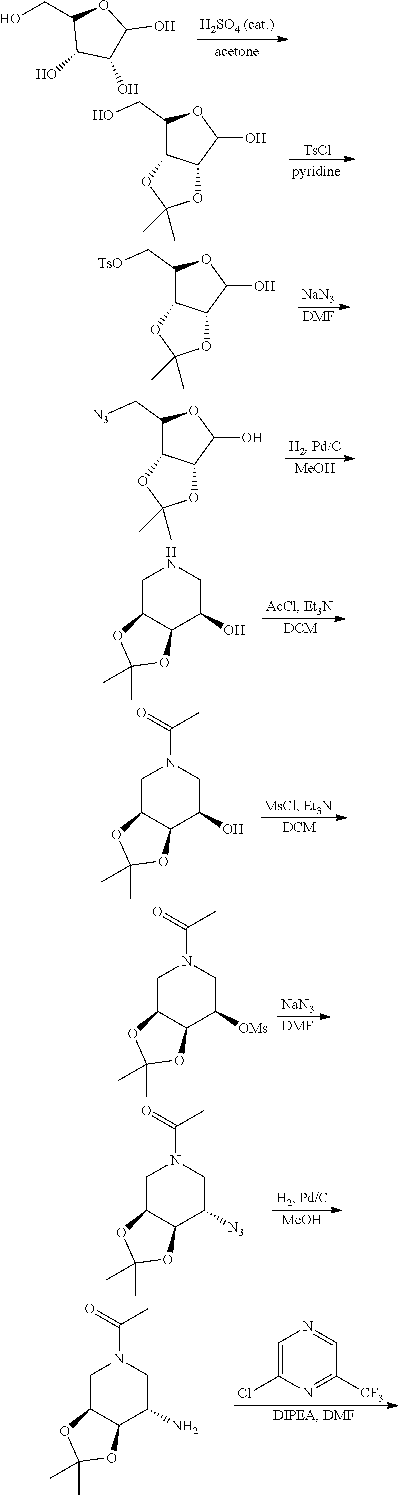

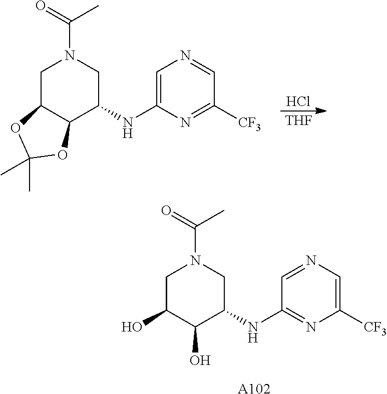 [SMILES: CC(=O)N1C[C@H](Nc2cncc(C(F)(F)F)n2)[C@H]2OC(C)(C)O[C@H]2C1 ; Cl ; "THF" ; CC(=O)N1C[C@H](Nc2cncc(C(F)(F)F)n2)[C@@H](O)[C@@H](O)C1 ; "A102"]

To a solution of (3R,4S,5S)-5-(hydroxymethyl)tetrahydrofuran-2,3,4-triol (1.00 eq) in acetone was added H₂SO₄ (0.10 eq) dropwise at 0° C. The reaction was stirred at 25° C. overnight. Saturated solution of NaHCO₃ and toluene were added. The mixture was concentrated in vacuo to remove the acetone from the mixture. The resulting mixture was diluted with ethyl acetate, washed with saturated solution of NaHCO₃, H₂O and brine. The organic layer was dried over Na₂SO₄ and concentrated in vacuo. The crude product was purified by flash chromatography (silica gel, 0-60% EA in PE) to give (3aR,6S,6aR)-6-(hydroxymethyl)-2,2-dimethyltetrahydrofuro[3,4-d][1,3]dioxol-4-ol as a colorless oil. LC-MS (ESI) found: 191 [M+H]⁺. ¹H NMR (400 MHz, DMSO-d₆): δ 6.47 (d, J=4.9 Hz, 1H), 5.16 (d, J=4.9 Hz, 1H), 4.90 (t, J=5.5 Hz, 1H), 4.68 (d, J=6.0 Hz, 1H), 4.43 (d, J=6.0 Hz, 1H), 3.99 (dd, J=7.1, 5.0 Hz, 1H), 3.46-3.37 (m, 2H), 1.36 (s, 3H), 1.24 (s, 3H).

### Step 2

A solution of (3aR,6S,6aR)-6-(hydroxymethyl)-2,2-dimethyltetrahydrofuro[3,4-d][1,3]dioxol-4-ol (1.00 eq) in pyridine was cooled to 0° C. and TsCl (1.50 eq) was added. The reaction was stirred at 25° C. for 2 hr. EA and aqueous HCl (1N) were added, the organic layer was separated and the aqueous layer was re-extracted with EA. The combined organic layers were washed with saturated solution of NaHCO₃ and brine, dried over Na₂SO₄ and concentrated in vacuo. The crude product was purified by flash chromatography (silica gel, 0˜100% EA in PE) to give ((3aR,4S,6aR)-6-hydroxy-2,2-dimethyltetrahydrofuro[3,4-d][1,3]dioxol-4-yl)methyl 4-methylbenzenesulfonate as a colorless oil. LC-MS (ESI) found: 345 [M+H]⁺.

### Step 3

A solution of ((3aR,4S,6aR)-6-hydroxy-2,2-dimethyltetrahydrofuro[3,4-d][1,3]dioxol-4-yl)methyl 4-methylbenzenesulfonate (1.00 eq) and sodium azide (3.00 eq) in DMF was stirred at 75° C. overnight. The resulting mixture was diluted with ethyl acetate. The organic layer was separated, washed with H₂O and brine, dried over Na₂SO₄ and concentrated in vacuo. The crude product was purified by flash chromatography (silica gel, 0-100% ethyl acetate in petroleum ether) to give (3aR,6S,6aR)-6-(azidomethyl)-2,2-dimethyltetrahydrofuro[3,4-d][1,3]dioxol-4-ol as a colorless oil. LC-MS (ESI) found: 216 [M+H]⁺. ¹H NMR (400 MHz, CDCl₃): δ 5.47 (d, J=4.9 Hz, 1H), 4.64 (t, J=5.1 Hz, 2H), 4.39-4.31 (m, 1H), 3.58 (dd, J=12.6, 7.0 Hz, 1H), 3.40 (dd, J=12.6, 5.7 Hz, 1H), 3.34 (d, J=5.0 Hz, 1H), 1.49 (s, 3H), 1.33 (s, 3H).

### Step 4

To a solution of (3aR,6S,6aR)-6-(azidomethyl)-2,2-dimethyltetrahydrofuro[3,4-d][1,3]dioxol-4-ol (1.00 eq) in MeOH was added Pd/C (10% purity) at 25° C., the reaction was stirred at 25° C. overnight under H2 (15 Psi). The mixture was filtered, the filtrate was concentrated in vacuo to afford (3aS,7R,7aR)-2,2-dimethylhexahydro-[1,3]dioxolo[4,5-c]pyridin-7-ol as a colorless oil. LC-MS (ESI) found: 174 [M+H]⁺.

### Step 5

To a solution of (3aS,7R,7aR)-2,2-dimethylhexahydro-[1,3]dioxolo[4,5-c]pyridin-7-ol (1.00 eq) in DCM was added Et₃N (2.00 eq), the mixture was cooled to 0° C., then acetyl chloride (1.00 eq) was added dropwise at 0° C. The solution was stirred at 25° C. for 3 hr. The mixture was diluted with DCM, the organic layer was separated, washed with H₂O and brine, then dried over Na₂SO₄ and concentrated in vacuo. The crude product was purified by flash chromatography (silica gel, 0˜10% MeOH in DCM) to give 1-((3aS,7R,7aR)-7-hydroxy-2,2-dimethyltetrahydro-[1,3]dioxolo[4,5-c]pyridin-5(4H)-yl)ethan-1-one as a colorless oil. LC-MS (ESI) found: 216 [M+H]⁺.

### Step 6

To a solution of 1-((3aS,7R,7aR)-7-hydroxy-2,2-dimethyltetrahydro-[1,3]dioxolo[4,5-c]pyridin-5(4H)-yl)ethan-1-one (1.00 eq) and Et₃N (3.00 eq) in DCM was added MsCl (1.50 eq) dropwise at 0° C. The solution was stirred at 25° C. for 2 h. The mixture was diluted with DCM, the organic layer was separated, then washed with H₂O and brine, dried over Na₂SO₄ and concentrated in vacuo. The crude product was purified by flash chromatography (silica gel, 0˜10% MeOH in DCM) to give (3aS,7R,7aS)-5-acetyl-2,2-dimethylhexahydro-[1,3]dioxolo[4,5-c]pyridin-7-yl methanesulfonate as a colorless oil. LC-MS (ESI) found: 294 [M+H]⁺.

### Step 7

A solution of (3aS,7R,7aS)-5-acetyl-2,2-dimethylhexahydro-[1,3]dioxolo[4,5-c]pyridin-7-yl methanesulfonate (1.00 eq) and sodium azide (3.00 eq) in DMF was stirred at 100° C. for 2 days. The resulting mixture was diluted with ethyl acetate, washed with H₂O and brine. The organic layer was separated, then dried over Na₂SO₄ and concentrated in vacuo. The crude product was purified by flash chromatography (silica gel, 0˜100% EA in PE) to give 1-((3aS,7S,7aR)-7-azido-2,2-dimethyltetrahydro-[1,3]dioxolo[4,5-c]pyridin-5(4H)-yl)ethan-1-one as a colorless oil. LC-MS (ESI) found: 241 [M+H]⁺.

### Step 8

To a solution of 1-((3aS,7S,7aR)-7-azido-2,2-dimethyltetrahydro-[1,3]dioxolo[4,5-c]pyridin-5(4H)-yl)ethan-1-one (1.00 eq) in MeOH was added Pd/C (10% purity), the mixture was stirred at 25° C. for 2 h under H2 (15 Psi). The resulting mixture was filtered, the filtrate was concentrated in vacuo to afford 1-((3aS,7S,7aR)-7-amino-2,2-dimethyltetrahydro-[1,3]dioxolo[4,5-c]pyridin-5(4H)-yl)ethan-1-one as a colorless oil. LC-MS (ESI) found: 215 [M+H]⁺.

### Step 9

To a solution of 1-((3aS,7S,7aR)-7-amino-2,2-dimethyltetrahydro-[1,3]dioxolo[4,5-c]pyridin-5(4H)-yl)ethan-1-one (1.00 eq) and 2-chloro-6-(trifluoromethyl)pyrazine (1.50 eq) in DMF was added DIEA (3.00 eq) at 25° C., the mixture was stirred at 100° C. overnight. The mixture was diluted with EA, washed with H₂O and brine, the organic layer was separated, dried over Na₂SO₄ and concentrated in vacuo. The crude product was purified by flash chromatography (silica gel, 0-10% MeOH in DCM) to give 1-((3aS,7S,7aR)-2,2-dimethyl-7-((6-(trifluoromethyl)pyrazin-2-yl)amino)tetrahydro-[1,3]dioxolo[4,5-c]pyridin-5(4H)-yl)ethan-1-one as a white solid. LC-MS (ESI) found: 361 [M+H]⁺.

### Step 10

To a solution of 1-((3aS,7S,7aR)-2,2-dimethyl-7-((6-(trifluoromethyl)pyrazin-2-yl)amino)tetrahydro-[1,3]dioxolo[4,5-c]pyridin-5(4H)-yl)ethan-1-one (1.00 eq) in THE was added HCl (10.0 eq, 2N). The reaction was stirred at 25° C. for 3 hr. The resulting mixture was filtered and concentrated in vacuo. The pH of the mixture was adjusted to 7 with saturated solution of sodium bicarbonate, then concentrated under reduced pressure. The crude product was purified by prep-HPLC to give 1-((3S,4R,5S)-3,4-dihydroxy-5-((6-(trifluoromethyl)pyrazin-2-yl)amino)piperidin-1-yl)ethan-1-one as a white solid. LC-MS (ESI) found: 321 [M+H]⁺. ¹H NMR (400 MHz, CD₃OD): δ 8.16 (d, J=24.1 Hz, 1H), 8.04 (d, J=18.0 Hz, 1H), 4.42-4.37 (m, 0.4H) 4.26 (dd, J=14.4, 4.1 Hz, 0.6H), 4.21-4.15 (m, 1H), 4.12 (dd, J=8.7, 4.0 Hz, 0.6H), 4.00-3.96 (m, 1H), 3.85-3.79 (m, 0.4H), 3.76 (ddd, J=9.0, 6.4, 3.1 Hz, 1H), 3.39 (dd, J=13.9, 2.3 Hz, 0.4H), 3.18 (dd, J=13.5, 2.2 Hz, 0.6H), 2.97 (ddd, J=33.4, 13.0, 8.7 Hz, 1H), 2.15 (d, J=8.2 Hz, 3H). ¹⁹F NMR (377 MHz, CD₃OD): δ −70.21 (s), −70.32 (s).

### Preparation of (2R, 3S, 4R,5S)-2-(hydroxymethyl)-5-[[4-(trifluoromethyl) pyrimidin-2-yl]amino]piperidine-3,4-diol

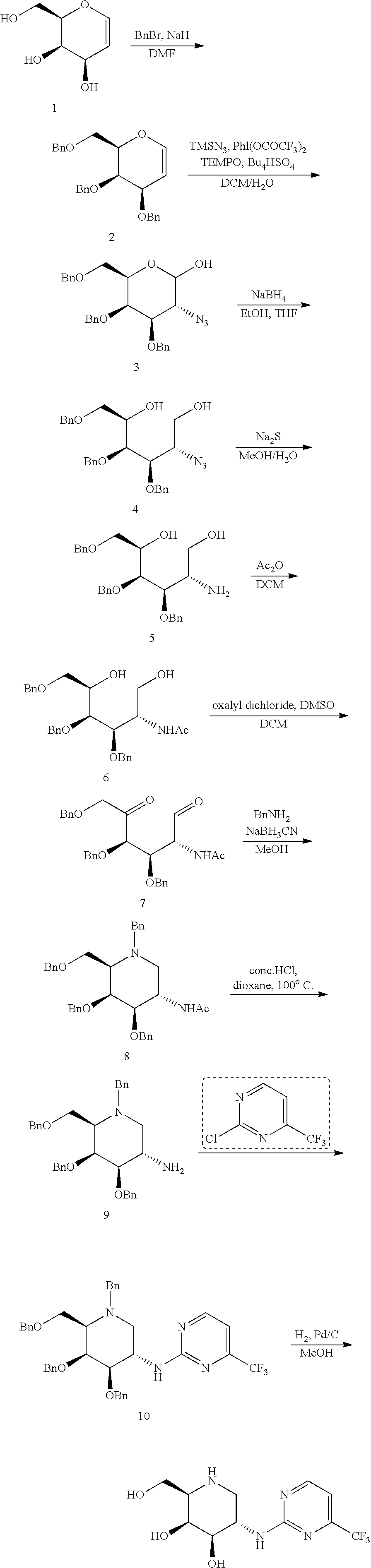

Step 1: To a solution of 2-(hydroxymethyl)-3,4-dihydro-2H-pyran-3,4-diol (37.2 g, 254.55 mmol, 1 eq.) in DMF (400 mL) was added NaH (45.81 g, 1.15 mol, 60% purity, 4.5 eq.) at 0° C., then added BnBr (152.38 g, 890.92 mmol, 105.82 mL, 3.5 eq.). The mixture was stirred at 20° C. for 1 hr. Several new peaks were shown on LC-MS desired compound was detected. The reaction mixture was quenched by sat. NH₄Cl (400 mL) at 0° C., extracted with MTBE (400 mL×3), dried over Na₂SO₄, filtered and concentrated under reduced pressure to give a residue. The residue was purified by column chromatography (SiO₂, Petroleum ether/Ethyl acetate=5:1, Rf=0.53). Compound 3,4-dibenzyloxy-2-(benzyloxymethyl)-3,4-dihydro-2H-pyran (91.86 g, 220.55 mmol, 86.64% yield) was obtained as a light yellow solid.
Step 2: To a solution of (2R,3R,4R)-3,4-dibenzyloxy-2-(benzyloxymethyl)-3,4-dihydro-2H-pyran (1.7 g, 4.08 mmol, 1 eq) and TMSN3 (1.41 g, 12.24 mmol, 1.61 mL, 3 eq) in DCM (10.5 mL) was added PIFA (3.51 g, 8.16 mmol, 2 eq), TEMPO (128.37 mg, 816.31 umol, 0.2 eq), Bu₄NHSO₄ (277.17 mg, 816.31 umol, 0.2 eq) and H₂O (3.68 g, 204.08 mmol, 3.68 mL, 50 eq) at 0° C. The mixture was stirred at 0° C. for 40 min. TLC (Petroleum ether:Ethyl acetate=3:1) indicated Reactant 1 was consumed completely and many new spots formed. LCMS showed desired mess was detected. The mixture was diluted with water 10 mL and extracted with DCM 30 mL (10 mL*3). The combined organic layers were dried over Na₂SO₄, filtered and concentrated under reduced pressure to give a residue. Compound (3R, 4R, 5R, 6R)-3-azido-4, 5-dibenzyloxy-6-(benzyloxymethyl) tetrahydropyran-2-ol (2 g, crude) was obtained as yellow oil.
Step 3: A mixture of (3R,4R,5R,6R)-3-azido-4,5-dibenzyloxy-6-(benzyloxymethyl) tetrahydropyran-2-ol (4.96 g, 10.43 mmol, 1 eq.) in EtOH (50 mL) was added NaBH₄ (2.15 g, 56.86 mmol, 5.45 eq.) at 0° C., and then the mixture was stirred at 20° C. for 1 hr. Several new peaks were shown on LC-MS desired compound was detected. The mixture was quenched by aq. NH₄Cl (2 mL) and concentrated under reduced pressure to remove the most solvent, then extracted with ethyl acetate (1 mL×3). The organic phase was dried over anhydrous Na₂SO₄ filtered and concentrated under reduced pressure to remove the solvent.

Compound (2S,3R,4S,5R)-2-azido-3,4,6-tribenzyloxy-hexane-1,5-diol (4.9 g, crude) was obtained as a yellow oil.
Step 4: A mixture of (2S,3R,4S,5R)-2-azido-3,4,6-tribenzyloxy-hexane-1,5-diol (6.5 g, 13.61 mmol, 1 eq.), Na₂S (5.31 g, 68.06 mmol, 2.86 mL, 5 eq.) in MeOH (65 mL) and H₂O (13 mL) was stirred at 70° C. for 12 hr. Several new peaks were shown on LC-MS desired compound was detected. The combined organic layers were filtered and concentrated under reduced pressure to give a residue.

Compound (2S,3R,4S,5R)-2-amino-3,4,6-tribenzyloxy-hexane-1,5-diol (6 g, crude) was obtained as a yellow oil.
Step 5: To a solution of (2S,3R,4S,5R)-2-amino-3,4,6-tribenzyloxy-hexane-1,5-diol (1 g, 2.21 mmol, 1 eq.) in DCM (10 mL) was added DIEA (572.44 mg, 4.43 mmol, 771.48 uL, 2 eq.) at 0° C., followed by Ac₂O (203.48 mg, 1.99 mmol, 186.67 uL, 0.9 eq.). The reaction mixture was stirred for 12 hrs at room temperature. Several new peaks were shown on LC-MS desired compound was detected. The reaction mixture was diluted with H₂O 10 mL and extracted with DCM 30 mL (10 mL*3). The combined organic layers were filtered and concentrated under reduced pressure to give a residue. The residue was purified by prep-TLC (SiO₂, Petroleum ether:Ethyl acetate=0:1, Rf=0.52).

Compound N-[(1S, 2R, 3S, 4R)-2, 3, 5-tribenzyloxy-4-hydroxy-1-(hydroxymethyl)pentyl]acetamide (1 g, 2.03 mmol, 91.48% yield) was obtained as a yellow oil.
Step 6: To a solution of oxalyl dichloride (1.50 g, 11.85 mmol, 1.04 mL, 4.5 eq) in DCM (10 mL) was added DMSO (1.23 g, 15.80 mmol, 1.23 mL, 6 eq) in DCM (10 mL) dropwised at −78° C. over 15 mins, and the mixture was stirred for 0.5 hr. N-[(1S,2R,3S,4R)-2,3,5-tribenzyloxy-4-hydroxy-1-(hydroxymethyl) pentyl]acetamide (1.3 g, 2.63 mmol, 1 eq) in DCM (10 mL) was added to the above mixture dropwised and stirred at −78° C. for 0.5 hr. Then the mixture was quenched by TEA (3.73 g, 36.87 mmol, 5.13 mL, 14 eq) at −70° C. Then the mixture was stirred at −70° C. for 0.5h, then warmed to 15° C. TLC (Petroleum ether:Ethyl acetate=0:1) showed one new spot was formed and reactant 1 was consumed. The mixture was washed by H₂O (50 mL) and aq. NaCl (10 mL×2). The organic phase was dried over anhydrous Na₂SO₄ and concentrated under reduced pressure to remove the part solvent. The crude product was used respectively for the next step without any purification.

Compound N-[(1R, 2R, 3R)-2, 3, 5-tribenzyloxy-1-formyl-4-oxo-pentyl]acetamide (1.3 g, crude) was obtained as yellow oil.
Step 7: To a solution of N-[(1R, 2R, 3R)-2,3,5-tribenzyloxy-1-formyl-4-oxo-pentyl]acetamide (1.20 g, 2.45 mmol, 1 eq) in MeOH (13 mL) was added dropwise BnNH₂ (288.29 mg, 2.69 mmol, 293.28 uL, 1.1 eq) at 25° C. After addition, NaBH₃CN (307.41 mg, 4.89 mmol, 2 eq) was added. The resulting mixture was stirred at 25° C. for 3 hr. TLC (Petroleum ether:Ethyl acetate=3:1) indicated Reactant 1 was consumed completely and one new spot formed. The reaction mixture was concentrated under reduced pressure to remove solvent. The residue was purified by column chromatography (SiO₂, Petroleum ether/Ethyl acetate=20/1 to 5/1).

Compound N-[(3S, 4R, 5S, 6R)-1-benzyl-4,5-dibenzyloxy-6-(benzyloxymethyl)-3-piperidyl]acetamide (1.1 g, 1.95 mmol, 79.64% yield) was obtained as a yellow oil.
Step 8: A mixture of N-[(3S,4R,5S,6R)-1-benzyl-4,5-dibenzyloxy-6-(benzyloxymethyl)-3-piperidyl]acetamide (300 mg, 531.24 umol, 1 eq) and HCl (12 M, 6.99 mL, 157.98 eq) in dioxane (1 mL) was stirred at 100° C. for 6 hr. TLC (Dichloromethane:Methanol=10:1) indicated Reactant 1 was consumed completely and one new spot formed. The reaction mixture was quenched by addition NaOH (aq) 2 mL at 0° C., and then diluted with water 5 mL and extracted with ethyl acetate 15 mL (5 mL*3). The combined organic layers were dried over Na₂SO₄, filtered and concentrated under reduced pressure to give a residue.

The residue was purified by prep-TLC (SiO2, DCM:MeOH=10:1).

Compound (3S, 4R, 5S, 6R)-1-benzyl-4,5-dibenzyloxy-6-(benzyloxymethyl)piperidin-3-amine (120 mg, 229.59 umol, 43.22% yield) was obtained as a yellow oil.
Step 9: To a solution of (3S,4R,5S,6R)-1-benzyl-4,5-dibenzyloxy-6-(benzyloxymethyl) piperidin-3-amine (100 mg, 191.32 umol, 1 eq) in DMSO (1 mL) was added 2-chloro-4-(trifluoromethyl)pyrimidine (69.84 mg, 382.65 umol, 46.25 uL, 2 eq) and DIEA (74.18 mg, 573.97 umol, 99.98 uL, 3 eq). The mixture was stirred at 100° C. for 12 hr. TLC (Dichloromethane: Methanol-7:1) indicated Reactant 1 was consumed completely and two new spots formed. The reaction mixture was partitioned between water 5 mL and ethyl acetate 4 mL. The organic phase was separated, dried over Na₂SO₄, filtered and concentrated under reduced pressure to give a residue.

Compound N-[(3S, 4R, 5S, 6R)-1-benzyl-4, 5-dibenzyloxy-6-(benzyloxymethyl)-3-piperidyl]-4-(trifluoromethyl) pyrimidin-2-amine (110 mg, crude) was obtained as a yellow oil was used into the next step directly.
Step 10: A mixture of N-[(3S,4R,5S,6R)-1-benzyl-4,5-dibenzyloxy-6-(benzyloxymethyl)-3-piperidyl]-4-(trifluoromethyl)pyrimidin-2-amine (100 mg, 149.53 umol, 1 eq) and Pd/C in MeOH (1 mL) was degassed and purged with H2 (15 psi) for 3 times, and then the mixture was stirred at 20° C. for 2 hr under H2 atmosphere. Several new peaks were shown on LC-MS desired compound was detected Compound (2R, 3S, 4R, 5S)-2-(hydroxymethyl)-5-[[4-(trifluoromethyl)pyrimidin-2-yl]amino]piperidine-3,4-diol (8.49 mg, 27.54 umol, 18.42% yield) was obtained as a colorless oil.

### Preparation of (2R,3S,4R,5S)-2-(hydroxymethyl)-5-[[6-(trifluoromethyl) pyrazin-2-yl]amino]piperidine-3,4-diol

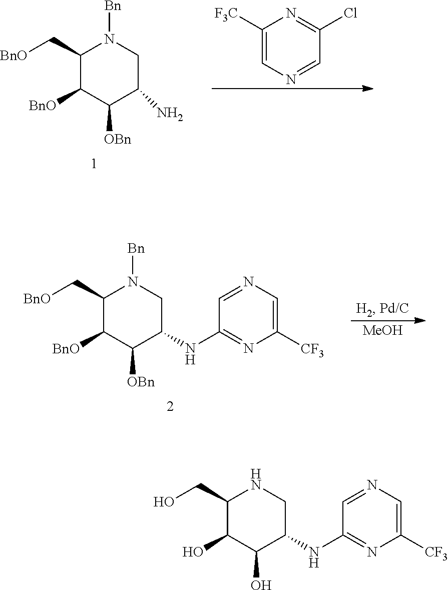 [SMILES: "MeOH" ; N[C@H]1CN(Cc2ccccc2)[C@H](COCc2ccccc2)[C@H](OCc2ccccc2)[C@@H]1OCc1ccccc1 ; FC(F)(F)c1cncc(Cl)n1 ; FC(F)(F)c1cncc(N[C@H]2CN(Cc3ccccc3)[C@H](COCc3ccccc3)[C@H](OCc3ccccc3)[C@@H]2OCc2ccccc2)n1 ; "H2, Pd/C" ; OC[C@H]1NC[C@H](Nc2cncc(C(F)(F)F)n2)[C@@H](O)[C@H]1O]

Step 1: A mixture of (3S,4R,5S,6R)-1-benzyl-4,5-dibenzyloxy-6-(benzyloxymethy) piperidin-3-amine (75 mg, 143.49 umol, 1 eq) and 2-chloro-6-(trifluoromethyl)pyrazine (78.58 mg, 430.48 umol, 3 eq), DIEA (55.64 mg, 430.48 umol, 74.98 uL, 3 eq) in DMSO (0.5 mL) was stirred at 90° C. for 12h. TLC(Petroleum ether:Ethyl acetate=0:1) showed reactant 1 was consumed and one new spot formed, the mixture was purified by prep-HPLC (TFA condition; column: Phenomenex luna C18 100*40 mm*5 um; mobile phase: [water(TFA)-ACN]; B %: 15%-60%, 8 min). Compound N-[(3S,4R,5S,6R)-1-benzyl-4,5-dibenzyloxy-6-(benzyloxymethyl)-3-piperidyl]-6-(trifluoromethyl)pyrazin-2-amine (54 mg, 74.97 umol, 52.25% yield, 92.85% purity) was obtained as a brown solid.
Step 2: To a solution of N-[(3S,4R,5S,6R)-1-benzyl-4,5-dibenzyloxy-6-(benzyloxymethyl) 3-piperidyl]-6-(trifluoromethyl)pyrazin-2-amine (50 mg, 74.77 umol, 1 eq) in MeOH (5 mL) was added Pd/C (10 mg, 10% purity) under N₂. The suspension was degassed under vacuum and purged with H2 several times. The mixture was stirred under H2 (15 psi) at 20° C. for 1 hours. LCMS showed starting material was consumed and one main peak of desired compound was detected. The mixture was concentrated to give a crude product. The residue was purified by prep-HPLC (basic condition column: Phenomenex C18 75*30 mm*3 um; mobile phase: [water(NH3H2O+NH4HCO3)-ACN]; B %: 1%-30%, 8 min)

Compound (2R,3S,4R,5S)-2-(hydroxymethyl)-5-[[6-(trifluoromethyl)pyrazin-2-yl]amino]piperidine-3,4-diol (11.27 mg, 36.44 umol, 48.74% yield, 99.6% purity) was obtained as a white solid.

¹H NMR (400 MHz, METHANOL-d4) δ ppm 8.12 (s, 1H) 7.98 (s, 1H) 4.23 (td, J=10.63, 4.88 Hz, 1H) 4.00 (d, J=1.50 Hz, 1H) 3.61-3.71 (m, 2H) 3.57 (dd, J=10.51, 2.88 Hz, 1H) 3.32-3.35 (m, 1H) 2.71 (t, J=6.44 Hz, 1H) 2.39 (dd, J=12.38, 11.26 Hz, 1H)

### Preparation of 2-(2-(2-((((2R,3R,4R,5S)-3,4-dihydroxy-5-((6-(trifluoromethyl)pyridin-2-yl)amino)tetrahydro-2H-pyran-2-yl)methyl)amino)-2-oxoethoxy)ethoxy)acetic acid (A103)

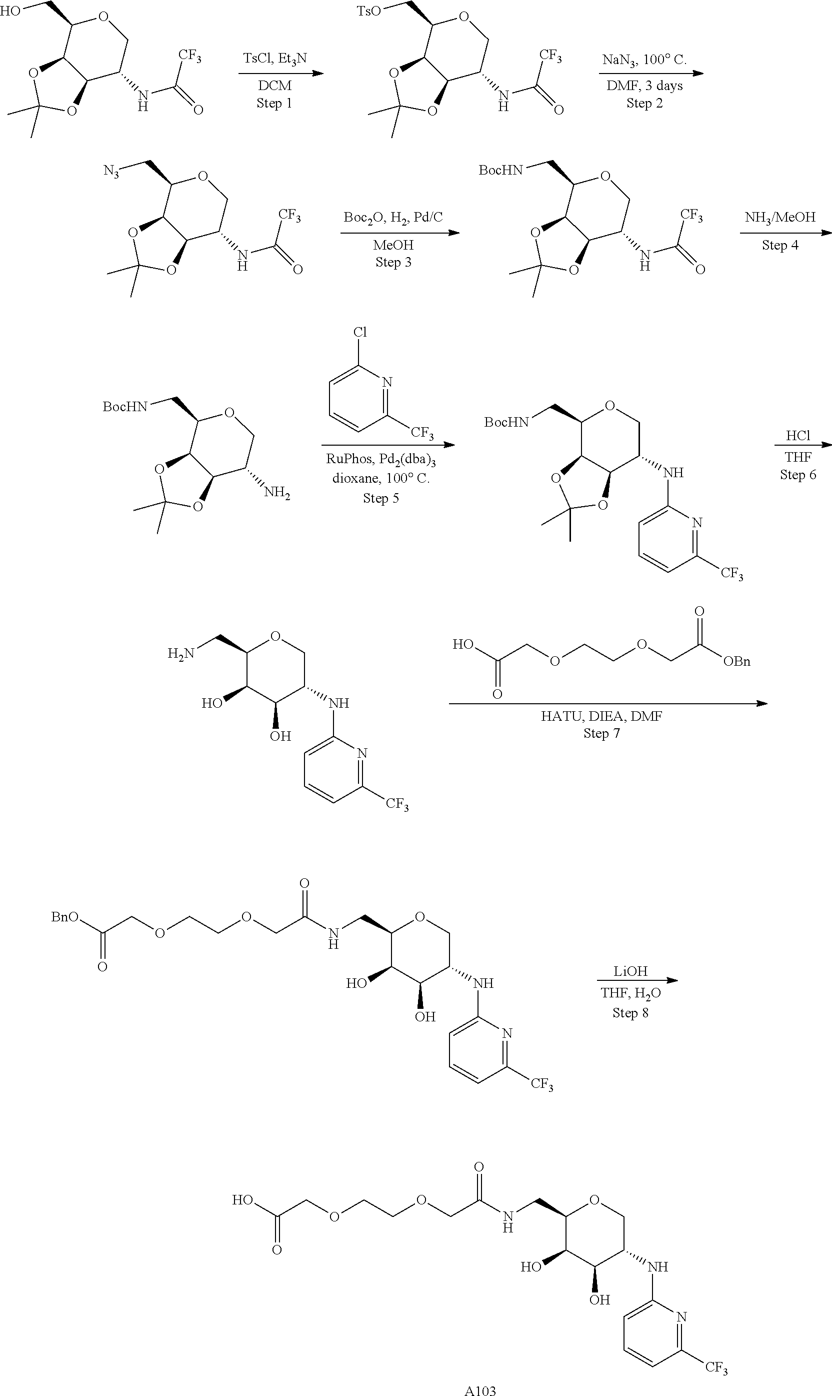

### Step 1

To a solution of 2,2,2-trifluoro-N-((3aR,4R,7S,7aR)-4-(hydroxymethyl)-2,2-dimethyltetrahydro-4H-[1,3]dioxolo[4,5-c]pyran-7-yl)acetamide (1.00 eq) in DCM were added Et₃N (3.00 eq) and TsCl (1.20 eq) at 25° C. The mixture was stirred at 25° C. overnight. The mixture was concentrated and the residue was purified by silica gel chromatography to afford ((3aR,4R,7S,7aR)-2,2-dimethyl-7-(2,2,2-trifluoroacetamido)tetrahydro-4H-[1,3]dioxolo[4,5-c]pyran-4-yl)methyl 4-methylbenzenesulfonate as a white solid. Chemical Formula: C₁₈H₂₂F₃NO₇S, LCMS found: [M+H]⁺=454.

### Step 2

To a solution of ((3aR,4R,7S,7aR)-2,2-dimethyl-7-(2,2,2-trifluoroacetamido)tetrahydro-4H-[1,3]dioxolo[4,5-c]pyran-4-yl)methyl 4-methylbenzenesulfonate (1.00 eq) in DMF was added NaN₃ (10.00 eq) at 25° C. The mixture was stirred at 100° C. for 3 days. Then the reaction was quenched with brine and extracted with EA. The organic layer was washed with brine, dried over anhydrous Na₂SO₄, filtered and concentrated, the residue was purified by silica gel chromatography to afford N-((3aS,4R,7S,7aR)-4-(azidomethyl)-2,2-dimethyltetrahydro-4H-[1,3]dioxolo[4,5-c]pyran-7-yl)-2,2,2-trifluoroacetamide as a yellow solid. Chemical Formula: C₁₁H₁₅F₃N₄O₄, LCMS found: [M+H]⁺=325.

### Step 3

To a solution of N-((3aS,4R,7S,7aR)-4-(azidomethyl)-2,2-dimethyltetrahydro-4H-[1,3]dioxolo[4,5-c]pyran-7-yl)-2,2,2-trifluoroacetamide (1.00 eq) in MeOH was added Pd/C (10% purity) and Boc₂O (2.00 eq), the mixture was stirred at 25° C. overnight under hydrogen atmosphere (15 psi). Then the mixture was filtered, the filtrate was concentrated under reduced pressure, the residue was purified by silica gel chromatography to give tert-butyl (((3aS,4R,7S,7aR)-2,2-dimethyl-7-(2,2,2-trifluoroacetamido)tetrahydro-4H-[1,3]dioxolo[4,5-c]pyran-4-yl)methyl)carbamate as colorless oil. Chemical Formula: C₁₆H₂₅F₃N₂O₆, LCMS found: [M+H]⁺=399.

### Step 4

A solution of tert-butyl (((3aS,4R,7S,7aR)-2,2-dimethyl-7-(2,2,2-trifluoroacetamido)tetrahydro-4H-[1,3]dioxolo[4,5-c]pyran-4-yl)methyl)carbamate (1.00 eq) in NH₃/MeOH (7 N) was stirred at 80° C. overnight in sealed tube. Then the mixture was concentrated and the residue was purified by silica gel chromatography to afford tert-butyl (((3aS,4R,7S,7aR)-7-amino-2,2-dimethyltetrahydro-4H-[1,3]dioxolo[4,5-c]pyran-4-yl)methyl)carbamate as a yellow oil. Chemical Formula: C₁₄H₂₆N₂O₅, LCMS found: [M+H]⁺=303.

### Step 5

To a solution of tert-butyl (((3aS,4R,7S,7aR)-7-amino-2,2-dimethyltetrahydro-4H-[1,3]dioxolo[4,5-c]pyran-4-yl)methyl)carbamate (1.00 eq) and 2-bromo-6-(trifluoromethyl)pyridine (1.20 eq) in toluene was added Cs₂CO₃ (2.00 eq), Pd₂(dba)₃ (0.20 eq) and RuPhos (0.20 eq). The mixture was stirred at 100° C. overnight under nitrogen atmosphere. The mixture was concentrated and the residue was purified by silica gel chromatography to afford tert-butyl (((3aS,4R,7S,7aR)-2,2-dimethyl-7-((6-(trifluoromethyl)pyridin-2-yl)amino)tetrahydro-4H-[1,3]dioxolo[4,5-c]pyran-4-yl)methyl)carbamate as a yellow solid. Chemical Formula: C₂₀H₂₈F₃N₃O₅, LCMS found: [M+H]⁺=448.

### Step 6

To a solution of tert-butyl (((3aS,4R,7S,7aR)-2,2-dimethyl-7-((6-(trifluoromethyl)pyridin-2-yl)amino)tetrahydro-4H-[1,3]dioxolo[4,5-c]pyran-4-yl)methyl)carbamate (1.00 eq) in THE was added HCl (10.0 eq, 2N), the mixture was stirred at 25° C. for 1 h. The mixture was concentrated to afford (2R,3R,4R,5S)-2-(aminomethyl)-5-((6-(trifluoromethyl)pyridin-2-yl)amino)tetrahydro-2H-pyran-3,4-diol as a yellow oil. Chemical Formula: C₁₂H₁₆F₃N₃O₃, LCMS found: [M+H]⁺=308.

### Step 7

To a solution of 2-(2-(2-(benzyloxy)-2-oxoethoxy)ethoxy)acetic acid (1.00 eq) in DMF was added HATU (1.20 eq) and DIEA (2.00 eq), the mixture was stirred at 25° C. for 0.5 h. Then (2R,3R,4R,5S)-2-(aminomethyl)-5-((6-(trifluoromethyl)pyridin-2-yl)amino)tetrahydro-2H-pyran-3,4-diol (1.00 eq) in DMF was added, the reaction mixture was stirred at 25° C. overnight. The reaction was diluted with water, then extracted with ethyl acetate, the organic layer was washed with brine, dried over anhydrous Na₂SO₄, filtered and concentrated, the residue was purified by silica gel chromatography to afford benzyl 2-(2-(2-((((2R,3R,4R,5S)-3,4-dihydroxy-5-((6-(trifluoromethyl)pyridin-2-yl)amino)tetrahydro-2H-pyran-2-yl)methyl)amino)-2-oxoethoxy)ethoxy)acetate as a yellow oil. Chemical Formula: C₂₅H₃₀F₃N₃O₈, LCMS found: [M+H]⁺=558.

### Step 8

To a solution of benzyl 2-(2-(2-((((2R,3R,4R,5S)-3,4-dihydroxy-5-((6-(trifluoromethyl)pyridin-2-yl)amino)tetrahydro-2H-pyran-2-yl)methyl)amino)-2-oxoethoxy)ethoxy)acetate (1.00 eq) in THE and H₂O was added LiOH (5.00 eq), the mixture was stirred at 25° C. for 0.5 h. The mixture was concentrated under reduced pressure, the residue was diluted with water, the pH of the aqueous phase was adjusted to 6 by HCl (1N), then extracted with EA, the organic layer was dried over anhydrous Na₂SO₄ and concentrated, the residue was purified by reverse phase (5-50% MeCN in water, 0.1% TFA) to afford 2-(2-(2-((((2R,3R,4R,5S)-3,4-dihydroxy-5-((6-(trifluoromethyl)pyridin-2-yl)amino)tetrahydro-2H-pyran-2-yl)methyl)amino)-2-oxoethoxy)ethoxy)acetic acid as a yellow solid. Chemical Formula: C₁₈H₂₄F₃N₃O₈, LCMS found: [M+H]⁺=468. 1H NMR (400 MHz, CD₃OD): δ 8.29 (s, 2H), 7.52 (t, J=7.8 Hz, 1H), 6.88 (d, J=7.2 Hz, 1H), 6.76 (d, J=8.4 Hz, 1H), 4.28 (s, 2H), 4.17 (dd, J=10.7, 4.8 Hz, 1H), 4.10 (d, J=10.7 Hz, 2H), 3.85 (s, 4H), 3.69 (dd, J=18.8, 7.0 Hz, 1H), 3.55 (dd, J=13.0, 10.2 Hz, 2H), 3.45 (dd, J=12.9, 7.5 Hz, 1H), 3.35 (s, 2H), 3.17-2.98 (m, 1H).

### Preparation of (2R,3R,4R,5R,6R)-2-(hydroxymethyl)-6-phenyl-5-((6-(trifluoromethyl) pyrazin-2-yl)amino)tetrahydro-2H-pyran-3,4-diol (A105)

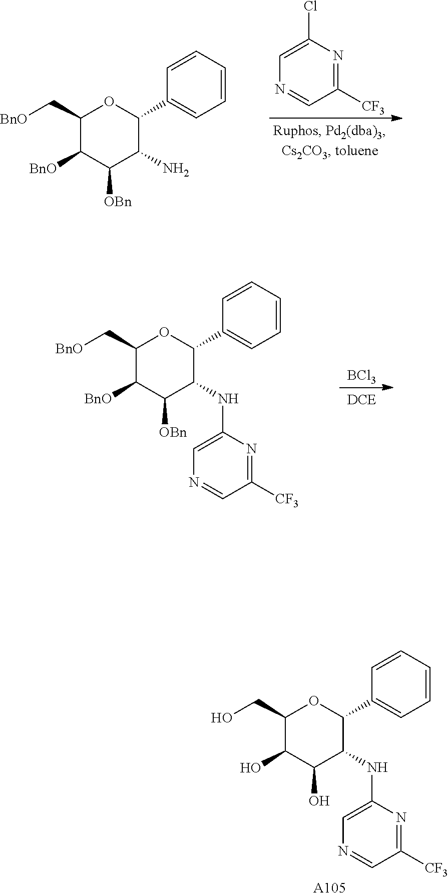

### Step 1

To a solution of (2R,3S,4R,5R,6R)-4,5-bis(benzyloxy)-6-((benzyloxy)methyl)-2-phenyltetrahydro-2H-pyran-3-amine (1.00 eq) and 2-chloro-6-(trifluoromethyl)pyrazine (1.50 eq) in toluene was added RuPhos (0.02 eq), Pd₂(dba)₃ (0.01 eq) and Cs₂CO₃ (2.00 eq), the mixture was stirred at 110° C. for 16 h under nitrogen atmosphere. The mixture was concentrated and the residue was purified by silica gel chromatography to afford N-((2R,3S,4R,5R,6R)-4,5-bis(benzyloxy)-6-((benzyloxy)methyl)-2-phenyltetrahydro-2H-pyran-3-yl)-6-(trifluoromethyl)pyrazin-2-amine as a colorless oil. Chemical Formula: C₃₈H₃₆F₃N₃O₄, LCMS found: [M+H]⁺=656.

### Step 2

To a solution of N-((2R,3S,4R,5R,6R)-4,5-bis(benzyloxy)-6-((benzyloxy)methyl)-2-phenyltetrahydro-2H-pyran-3-yl)-6-(trifluoromethyl)pyrazin-2-amine (1.00 eq) in DCE was added BCl₃ (5.00 eq) at −40° C. The mixture was stirred at 25° C. for 2 h, then quenched with MeOH. The mixture was concentrated and then purified by reverse phase (C18, 0-40% MeCN in water, 0.3% TFA) to afford (2R,3R,4R,5R,6R)-2-(hydroxymethyl)-6-phenyl-5-((6-(trifluoromethyl)pyrazin-2-yl)amino)tetrahydro-2H-pyran-3,4-diol as a white solid. Chemical Formula: C₁₇H₁₈F₃N₃O₄, LCMS found: [M+H]⁺=386. ¹H NMR (400 MHz, CD₃OD) δ 7.96 (s, 1H), 7.84 (s, 1H), 7.42 (d, J=7.4 Hz, 2H), 7.19 (t, J=7.5 Hz, 2H), 7.10 (t, J=7.3 Hz, 1H), 5.44 (d, J=2.5 Hz, 1H), 4.62 (dd, J=4.7, 2.8 Hz, 1H), 4.27 (dd, J=12.3, 8.2 Hz, 1H), 4.21 (dd, J=6.0, 3.2 Hz, 1H), 4.17 (dd, J=11.3, 5.6 Hz, 1H), 4.06 (dd, J=4.8, 3.2 Hz, 1H), 3.85 (dd, J=12.3, 2.8 Hz, 1H).

### Preparation of 2-(2-(2-((((2R,3R,4R,5S)-3,4-dihydroxy-5-((4-(trifluoromethyl)pyrimidin-2-yl)amino)tetrahydro-2H-pyran-2-yl)methyl)amino)-2-oxoethoxy)ethoxy)acetic acid (A106)

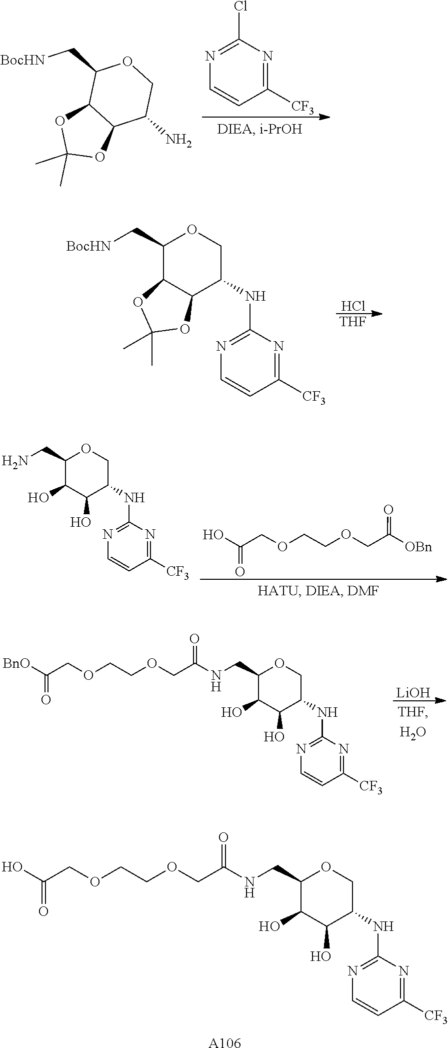

To a solution of tert-butyl (((3aS,4R,7S,7aR)-7-amino-2,2-dimethyltetrahydro-4H-[1,3]dioxolo[4,5-c]pyran-4-yl)methyl)carbamate (1.00 eq) and 2-chloro-4-(trifluoromethyl)pyrimidine (1.20 eq) in i-PrOH was added DIEA (2.00 eq), the mixture was stirred at 80° C. overnight under nitrogen. The mixture was concentrated under reduced pressure, the residue was purified by silica gel chromatography to afford tert-butyl (((3aS,4R,7S,7aR)-2,2-dimethyl-7-((4-(trifluoromethyl)pyrimidin-2-yl)amino)tetrahydro-4H-[1,3]dioxolo[4,5-c]pyran-4-yl)methyl)carbamate as a yellow solid. Chemical Formula: C₁₉H₂₇F₃N₄O₅, LCMS found: [M+H]⁺=449.

### Step 2

To a solution of tert-butyl (((3aS,4R,7S,7aR)-2,2-dimethyl-7-((4-(trifluoromethyl)pyrimidin-2-yl)amino)tetrahydro-4H-[1,3]dioxolo[4,5-c]pyran-4-yl)methyl)carbamate (1.00 eq) in THE was added HCl (10.0 eq, 2N), the mixture was stirred at 25° C. for 1 h. The mixture was concentrated to afford (2R,3R,4R,5S)-2-(aminomethyl)-5-((4-(trifluoromethyl)pyrimidin-2-yl)amino)tetrahydro-2H-pyran-3,4-diol as a yellow oil. Chemical Formula: C₁₁H₁₅F₃N₄O₃, LCMS found: [M+H]⁺=309.

### Step 3

To a solution of (2R,3R,4R,5S)-2-(aminomethyl)-5-((4-(trifluoromethyl)pyrimidin-2-yl)amino)tetrahydro-2H-pyran-3,4-diol (1.00 eq) in DMF was added HATU (1.20 eq) and DIEA (2.00 eq), the mixture was stirred at 25° C. for 0.5 h. Then 2-(2-(2-(benzyloxy)-2-oxoethoxy)ethoxy)acetic acid (1.00 eq) in DMF was added, the reaction mixture was stirred at 25° C. overnight. The reaction was diluted with water, then extracted with ethyl acetate, the organic layer was concentrated and the residue was purified by silica gel chromatography to afford benzyl 2-(2-(2-((((2R,3R,4R,5S)-3,4-dihydroxy-5-((4-(trifluoromethyl)pyrimidin-2-yl)amino)tetrahydro-2H-pyran-2-yl)methyl)amino)-2-oxoethoxy)ethoxy)acetate as a yellow oil. Chemical Formula: C₂₄H₂₉F₃N₄O₈, LCMS found: [M+H]⁺=559.

### Step 4

To a solution of benzyl 2-(2-(2-((((2R,3R,4R,5S)-3,4-dihydroxy-5-((4-(trifluoromethyl)pyrimidin-2-yl)amino)tetrahydro-2H-pyran-2-yl)methyl)amino)-2-oxoethoxy)ethoxy)acetate (1.00 eq) in THE and H₂O was added LiOH (5.00 eq), the mixture was stirred at 25° C. for 0.5 h. The mixture was concentrated under reduced pressure, the residue was diluted with water, the pH of the aqueous phase was adjusted to 6 by HCl (1N), then extracted with EA, the organic layer was dried over anhydrous Na₂SO₄ and concentrated, the residue was purified by reverse phase (5-45% MeCN in water, 0.1% TFA) to afford 2-(2-(2-((((2R,3R,4R,5S) 3,4-dihydroxy-5-((4-(trifluoromethyl)pyrimidin-2-yl)amino)tetrahydro-2H-pyran-2-yl)methyl)amino)-2-oxoethoxy)ethoxy)acetic acid as a yellow solid. Chemical Formula: C₁₇H₂₃F₃N₄O₈, LCMS found: [M+H]⁺=469. ¹H NMR (400 MHz, CD₃OD): δ 8.51 (d, J=4.7 Hz, 1H), 8.14 (s, 1H), 6.90 (d, J=4.9 Hz, 1H), 4.34 (dd, J=13.1, 8.0 Hz, 1H), 4.16-4.04 (m, 5H) 3.84 (d, J=3.2 Hz, 1H), 3.78 (s, 4H), 3.66 (dd, J=10.5, 2.9 Hz, 1H), 3.59-3.43 (m, 3H), 3.13 (t, J=10.9 Hz, 1H).

### Preparation of 2-(2-(2-((((2R,3R,4R,5S)-3,4-dihydroxy-5-((2-(trifluoromethyl)pyridin-4-yl)amino)tetrahydro-2H-pyran-2-yl)methyl)amino)-2-oxoethoxy)ethoxy)acetic acid (A107)

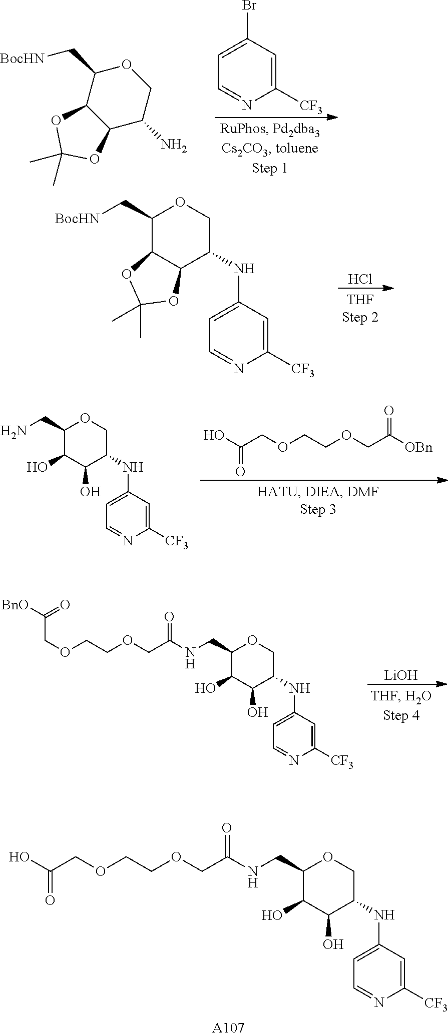

### Step 1

To a solution of tert-butyl (((3aS,4R,7S,7aR)-7-amino-2,2-dimethyltetrahydro-4H-[1,3]dioxolo[4,5-c]pyran-4-yl)methyl)carbamate (1.00 eq) and 4-bromo-2-(trifluoromethyl)pyridine (1.20 eq) in toluene was added RuPhos (0.20 eq), Pd₂(dba)₃ (0.20 eq) and Cs₂CO₃ (2.50 eq), the mixture was stirred at 100° C. overnight under nitrogen. The mixture was concentrated under reduced pressure, the residue was purified by silica gel chromatography to afford tert-butyl (((3aS,4R,7S,7aR)-2,2-dimethyl-7-((2-(trifluoromethyl)pyridin-4-yl)amino)tetrahydro-4H-[1,3]dioxolo[4,5-c]pyran-4-yl)methyl)carbamate as a yellow solid. Chemical Formula: C₂₀H₂₈F₃N₃O₅, LCMS found: [M+H]⁺=448.

### Step 2

To a solution of tert-butyl (((3aS,4R,7S,7aR)-2,2-dimethyl-7-((2-(trifluoromethyl)pyridin-4-yl)amino)tetrahydro-4H-[1,3]dioxolo[4,5-c]pyran-4-yl)methyl)carbamate (1.00 eq) in THF was added HCl (10.0 eq, 2N), and the mixture was stirred at 25° C. for 1 h. The mixture was concentrated to afford (2R,3R,4R,5S)-2-(aminomethyl)-5-((2-(trifluoromethyl)pyridin-4-yl)amino)tetrahydro-2H-pyran-3,4-diol as a yellow oil. Chemical Formula: C₁₂H₁₆F₃N₃O₃, LCMS found: [M+H]⁺=308.

### Step 3

To a solution of (2R,3R,4R,5S)-2-(aminomethyl)-5-((2-(trifluoromethyl)pyridin-4-yl)amino)tetrahydro-2H-pyran-3,4-diol (1.00 eq) in DMF was added HATU (1.20 eq) and DIEA (2.00 eq), the mixture was stirred at 25° C. for 0.5 h. Then 2-(2-(2-(benzyloxy)-2-oxoethoxy)ethoxy)acetic acid (1.00 eq) in DMF was added, the reaction mixture was stirred at 25° C. overnight. The reaction was diluted with water, then extracted with ethyl acetate, the organic layer was concentrated and the residue was purified by silica gel chromatography to afford benzyl 2-(2-(2-((((2R,3R,4R,5S)-3,4-dihydroxy-5-((2-(trifluoromethyl)pyridin-4-yl)amino)tetrahydro-2H-pyran-2-yl)methyl)amino)-2-oxoethoxy)ethoxy)acetate as a yellow oil. Chemical Formula: C₂₅H₃₀F₃N₃O₈, LCMS found: [M+H]⁺=558.

### Step 4

To a solution of benzyl 2-(2-(2-((((2R,3R,4R,5S)-3,4-dihydroxy-5-((2-(trifluoromethyl)pyridin-4-yl)amino)tetrahydro-2H-pyran-2-yl)methyl)amino)-2-oxoethoxy)ethoxy)acetate (1.00 eq) in THE and H₂O was added LiOH (5.00 eq), the mixture was stirred at 25° C. for 0.5 h. The mixture was concentrated under reduced pressure, the residue was diluted with water, the pH of the aqueous phase was adjusted to 6 by HCl (1N), then extracted with ethyl acetate, the organic layer was dried over anhydrous Na₂SO₄ and concentrated, the residue was purified by reverse phase (5-40% MeCN in water, 0.1% TFA) to afford 2-(2-(2-((((2R,3R,4R,5S)-3,4-dihydroxy-5-((2-(trifluoromethyl)pyridin-4-yl)amino)tetrahydro-2H-pyran-2-yl)methyl)amino)-2-oxoethoxy)ethoxy)acetic acid as a yellow solid. Chemical Formula: C₁₈H₂₄F₃N₃O₈, LCMS found: [M+H]⁺=468. ¹H NMR (400 MHz, CD₃OD): δ 8.10 (s, 1H), 8.09 (s, 1H), 7.01 (d, J=2.3 Hz, 1H), 6.79 (dd, J=5.9, 2.3 Hz, 1H), 4.13 (s, 2H), 4.05 (s, 2H), 3.99-3.81 (m, 3H), 3.74 (s, 4H) 3.59-3.49 (m, 3H), 3.48-3.41 (m, 1H), 3.10 (t, J=10.7 Hz, 1H).

### Preparation of (2R,3R,4R,5S)-2-(hydroxymethyl)-5-(phenylamino)tetrahydro-2H-pyran-3,4-diol (A108)

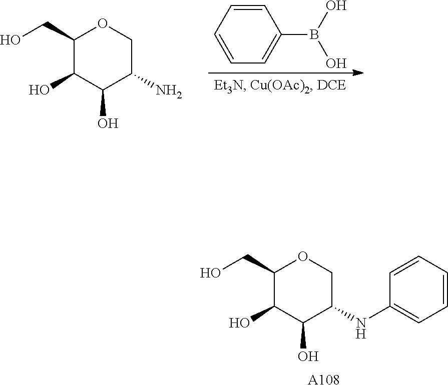

To a solution of (2R,3R,4R,5S)-5-amino-2-(hydroxymethyl)tetrahydro-2H-pyran-3,4-diol (1.00 eq) and phenylboronic acid (2.00 eq) in DCE (10 mL) was added Cu(OAc)₂ (5.00 eq) and Et₃N (5.00 eq), the mixture was degassed and purged with nitrogen several times, then stirred at 25° C. for 16 hours under nitrogen atmosphere. The mixture was filtered, the filtrate was concentrated under reduced pressure. The residue was purified by silica gel chromatography (DCM/MeOH=30/1 to 10/1) to afford the crude product. The obtained crude product was further purified by reverse phase (C18, 5-40% MeCN in water, 0.2% TFA) to afford A108 as a colorless oil. LCMS found: [M+H]⁺=240. ¹H NMR (400 MHz, CD₃OD): δ 7.08 (t, J=7.2 Hz, 2H), 6.70 (d, J=7.6 Hz, 2H), 6.59 (t, J=7.2 Hz, 1H), 4.07 (dd, J=11.2, 5.2 Hz, 1H), 3.91 (d, J=2.8 Hz, 1H), 3.81-3.67 (m, 3H), 3.52 (dd, J=10.0, 3.2 Hz, 1H), 3.47-3.42 (m, 1H), 3.02 (t, J=10.8 Hz, 1H).

### Preparation of (2R,3R,4R,5R,6S)-2-(hydroxymethyl)-6-phenyl-5-((6-(trifluoromethyl)pyrazin-2-yl)amino)tetrahydro-2H-pyran-3,4-diol (A109)

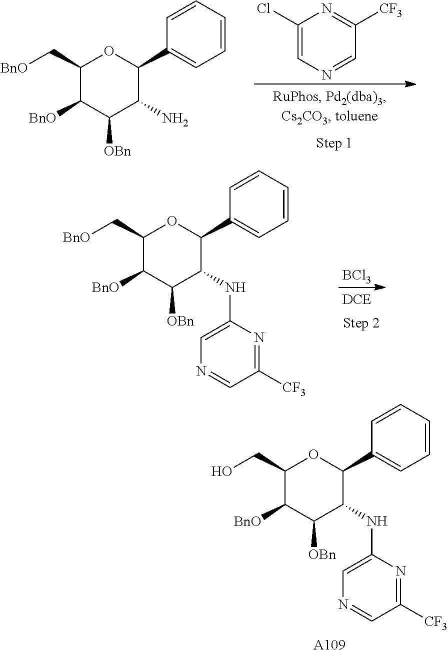

Step 1: A solution of (2S,3S,4R,5R,6R)-4,5-bis(benzyloxy)-6-((benzyloxy)methyl)-2-phenyltetrahydro-2H-pyran-3-amine (1.00 eq), 2-chloro-6-(trifluoromethyl)pyrazine (1.50 eq), RuRhos (0.2 eq), Pd₂(dba)₃ (0.1 eq) and Cs₂CO₃ (2.00 eq) in toluene was stirred at 110° C. for 16 h. The mixture was concentrated and purified by reverse phase (C18, 0-90% MeCN in water, 0.3% TFA) to afford N-((2S,3S,4R,5R,6R)-4,5-bis(benzyloxy)-6-((benzyloxy)methyl)-2-phenyltetrahydro-2H-pyran-3-yl)-6-(trifluoromethyl)pyrazin-2-amine as colorless oil. LCMS found: [M+H]⁺=656.
Step 2: To a solution of N-((2S,3S,4R,5R,6R)-4,5-bis(benzyloxy)-6-((benzyloxy)methyl)-2-phenyltetrahydro-2H-pyran-3-yl)-6-(trifluoromethyl)pyrazin-2-amine (1.00 eq) in DCE was added BCl₃ (5.00 eq) dropwise at −40° C. The mixture was stirred at rt for 2 h, then quenched with MeOH. The mixture was concentrated and then purified by reverse phase (C18, 0-40% MeCN in water, 0.3% TFA) to afford A109 as a white solid. LCMS found: [M+H]⁺=386. ¹H NMR (400 MHz, CD₃OD): δ 8.05 (s, 1H), 7.90 (s, 1H), 7.50 (d, J=7.6 Hz, 2H), 7.28 (t, J=7.5 Hz, 2H), 7.20 (t, J=7.3 Hz, 1H), 4.97 (d, J=5.7 Hz, 1H), 4.75 (s, 1H), 4.12 (dd, J=12.0, 7.6 Hz, 1H), 4.01-3.98 (m, 1H), 3.92 (dd, J=7.5, 3.8 Hz, 1H), 3.88 (t, J=3.3 Hz, 1H), 3.81 (dd, J=12.0, 3.6 Hz, 1H)

### Preparation of 2-(2-(2-((((2R,3R,4R,5S)-5-((3-fluoro-6-(trifluoromethyl)pyridin-2-yl)amino) 3,4-dihydroxytetrahydro-2H-pyran-2-yl)methyl)amino)-2-oxoethoxy)ethoxy)acetic acid (A110)

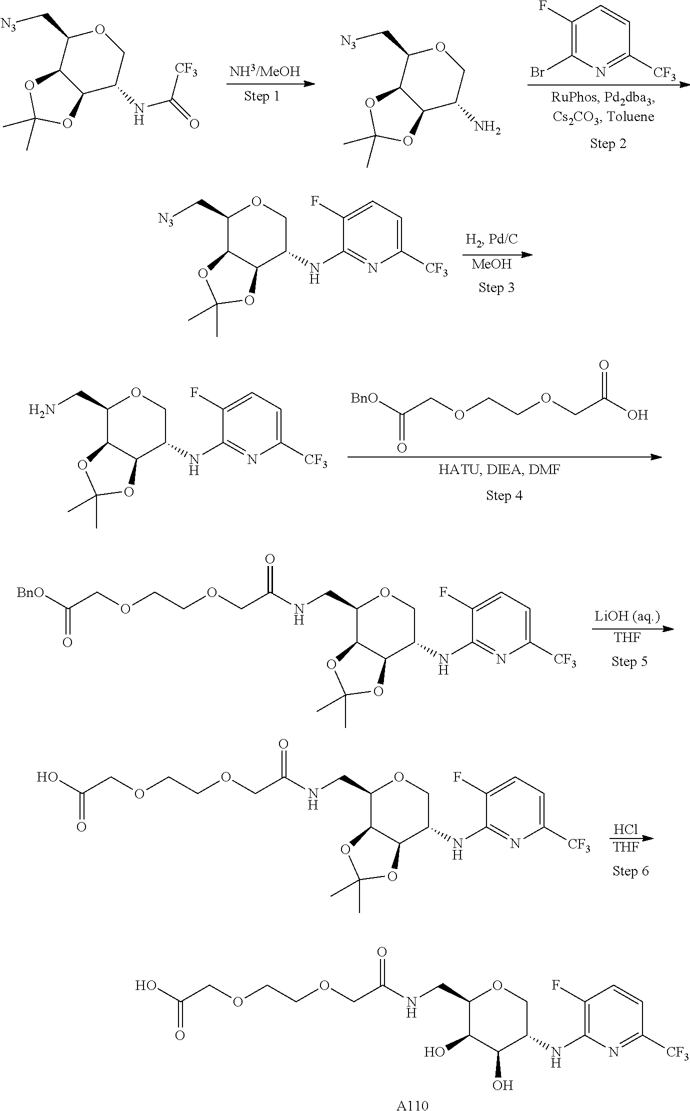

Step 1: To a solution of N-((3aS,4R,7S,7aR)-4-(azidomethyl)-2,2-dimethyltetrahydro-4H-[1,3]dioxolo[4,5-c]pyran-7-yl)-2,2,2-trifluoroacetamide (1.00 eq) in MeOH (10 mL) was added NH₃/MeOH (3.00 eq) at rt. The mixture was stirred at 80° C. for 12 hours. The mixture was concentrated under reduced pressure and the residue was diluted with water, extracted with ethyl acetate. The combined organic layers were washed with brine, dried over Na₂SO₄, filtered and concentrated under reduced pressure. The residue was purified by silica gel chromatography (1-10% MeOH in DCM) to afford (3aS,4R,7S,7aR)-4-(azidomethyl)-2,2-dimethyltetrahydro-4H-[1,3]dioxolo[4,5-c]pyran-7-amine as a white solid. LCMS found. [M+H]⁺=229.
Step 2: To a solution of (3aS,4R,7S,7aR)-4-(azidomethyl)-2,2-dimethyltetrahydro-4H-[1,3]dioxolo[4,5-c]pyran-7-amine (1.00 eq) in toluene (10 mL) was added 2-bromo-3-fluoro-6-(trifluoromethyl)pyri dine (1.20 eq), Cs₂CO₃ (1.5 eq), Pd₂dba₃ (0.1 eq) and RuPhos (0.1 eq) at rt. The mixture was stirred at 110° C. under N₂ for 3 hours. The mixture was concentrated, the residue was diluted with water, extracted with ethyl acetate. The combined organic layers were washed with brine, dried over Na₂SO₄, filtered and concentrated under reduced pressure. The residue was purified by silica gel column chromatography (5-10% MeOH in DCM) to afford N-((3aS,4R,7S,7aR)-4-(azidomethyl)-2,2-dimethyltetrahydro-4H-[1,3]dioxolo[4,5-c]pyran-7-yl)-3-fluoro-6-(trifluoromethyl)pyridin-2-amine as a white solid. LCMS found: [M+H]⁺=392.
Step 3: To a mixture of N-((3aS,4R,7S,7aR)-4-(azidomethyl)-2,2-dimethyltetrahydro-4H-[1,3]dioxolo[4,5-c]pyran-7-yl)-3-fluoro-6-(trifluoromethyl)pyridin-2-amine (1.00 eq) in MeOH was added Pd/C (10% purity), the mixture was degassed and purged with hydrogen several times, then stirred at 25° C. under H2 (15 psi) atmosphere for 0.5 hour. The mixture was filtered and the filtrate was concentrated under reduced pressure to afford N-((3aS,4R,7S,7aR)-4-(aminomethyl) 2,2-dimethyltetrahydro-4H-[1,3]dioxolo[4,5-c]pyran-7-yl)-3-fluoro-6-(trifluoromethyl)pyridin-2-amine as a yellow oil.
Step 4: To a solution of 2-(2-(2-(benzyloxy)-2-oxoethoxy)ethoxy)acetic acid (1.20 eq) in DMF (10 mL) was added DIEA (3.00 eq) and HATU (1.50 eq), the mixture was stirred at 25° C. for 0.5 h, then N-((3aS,4R,7S,7aR)-4-(aminomethyl)-2,2-dimethyltetrahydro-4H-[1,3]dioxolo[4,5-c]pyran-7-yl)-3-fluoro-6-(trifluoromethyl)pyridin-2-amine (1.00 eq) was added, the resulting mixture was stirred at 25° C. for 5 hours. The mixture was diluted with water, extracted with ethyl acetate. The combined organic layers were washed with brine, dried over Na₂SO₄, filtered and concentrated under reduced pressure. The residue was purified by silica gel column chromatography (5-15% MeOH in DCM) to afford benzyl 2-(2-(2-((((3aS,4R,7S,7aR)-7-((3-fluoro-6-(trifluoromethyl)pyridin-2-yl)amino)-2,2-dimethyltetrahydro-4H-[1,3]dioxolo[4,5-c]pyran-4-yl)methyl)amino)-2-oxoethoxy)ethoxy)acetate as a yellow oil. LCMS found: [M+H]⁺=616.
Step 5: To a solution of benzyl 2-(2-(2-((((3aS,4R,7S,7aR)-7-((3-fluoro-6-(trifluoromethyl)pyridin-2-yl)amino)-2,2-dimethyltetrahydro-4H-[1,3]dioxolo[4,5-c]pyran-4-yl)methyl)amino)-2-oxoethoxy)ethoxy)acetate (1.00 eq) in THF (10 mL) and H₂O (3 mL) was added LiOH (5.00 eq), the mixture was stirred at 25° C. for 2 hours. The mixture was concentrated under reduced pressure to afford 2-(2-(2-((((3aS,4R,7S,7aR)-7-((3-fluoro-6-(trifluoromethyl)pyridin-2-yl)amino)-2,2-dimethyltetrahydro-4H-[1,3]dioxolo[4,5-c]pyran-4-yl)methyl)amino)-2-oxoethoxy)ethoxy)acetic acid as a colorless oil. LCMS found: [M+H]⁺=526.
Step 6: To a solution of 2-(2-(2-((((3aS,4R,7S,7aR)-7-((3-fluoro-6-(trifluoromethyl)pyridin-2-yl)amino)-2,2-dimethyltetrahydro-4H-[1,3]dioxolo[4,5-c]pyran-4-yl)methyl)amino)-2-oxoethoxy)ethoxy)acetic acid (1.00 eq) in THF (10 mL) was added HCl (3 mL, 1 N), the mixture was stirred at 25° C. for 2 hours. The mixture was concentrated under reduced pressure and the residue was purified by prep-HPLC to afford compound A110 as a white solid. LCMS found: [M+H]⁺=486. ¹H NMR (400 MHz, CD₃OD): δ 7.35 (dd, J=10.7, 8.0 Hz, 1H), 6.93 (dd, J=8.0, 2.9 Hz, 1H), 4.45 (td, J=10.6, 5.2 Hz, 1H), 4.15 (dd, J=11.2, 5.2 Hz, 1H), 4.02 (s, 2H), 3.93 (s, 2H), 3.89 (d, J=3.2 Hz, 1H), 3.74 (dd, J=11.2, 3.2 Hz, 1H), 3.72-3.66 (m, 4H), 3.58-3.54 (m, 1H), 3.52-3.47 (m, 2H), 3.16 (t, J=10.8 Hz, 1H).

### Preparation of 4-(N-(((2R,3R,4R,5S)-3,4-dihydroxy-5-((6-(trifluoromethyl)pyrazin-2-yl)amino)tetrahydro-2H-pyran-2-yl)methyl)sulfamoyl)benzoic acid (A111)

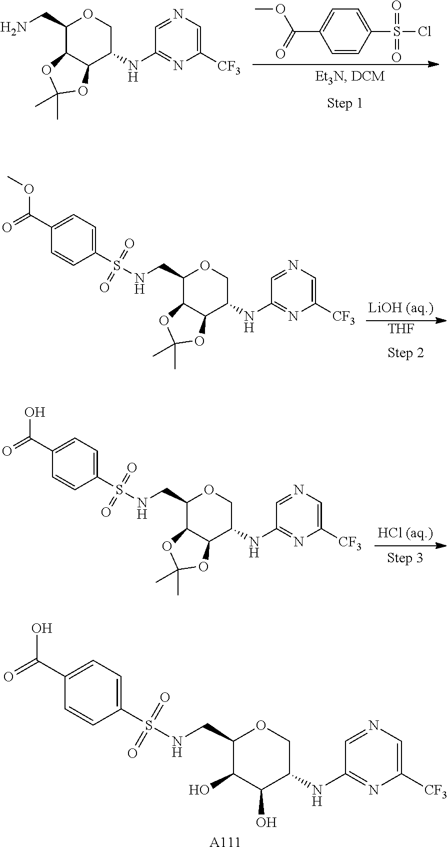

Step 1: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.00 eq) in DCM (10 mL) were added methyl 4-(chlorosulfonyl)benzoate (1.20 eq) and Et₃N (3.00 eq). The mixture was stirred at 25° C. for 3 hours. Then the mixture was concentrated and the residue was purified by silica gel chromatography (30% ethyl acetate in petroleum ether) to afford methyl 4-(N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)sulfamoyl)benzoate as yellow oil. LCMS found: [M+H]⁺=547.
Step 2: To a solution of methyl 4-(N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)sulfamoyl)benzoate (1.00 eq) in THF (10 mL) and water (2 mL) was added LiOH (5.00 eq). The mixture was stirred at 25° C. for 3 hours, then the mixture was evaporated to afford 4-(N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)sulfamoyl)benzoic acid as a white solid. LCMS found: [M+H]⁺=533.
Step 3: To a solution of 4-(N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)sulfamoyl)benzoic acid (1.00 eq) in THF (10 mL) was added HCl (1 mL, 2 N). The mixture was stirred at 25° C. for 4 hours, then concentrated. The residue was purified by prep-HPLC to afford A111 as a white solid. LCMS found: [M+H]⁺=493. ¹H NMR (400 MHz, DMSO-d₆): δ 8.19 (s, 1H), 8.13 (d, J=8.4 Hz, 2H) 8.06 (s, 1H), 7.95-7.89 (m, 3H), 7.58 (d, J=8.0 Hz, 1H), 4.86 (d, J=6.0 Hz, 1H), 4.76 (d, J=4.8 Hz, 1H), 4.07-3.93 (m, 1H), 3.81 (dd, J=10.9, 5.0 Hz, 1H), 3.68-3.63 (m, 1H), 3.51-3.49 (m, 1H), 3.30-3.27 (m, 1H), 2.97 (t, J=6.1 Hz, 2H), 2.82 (t, J=10.9 Hz, 1H).

### Preparation of N-(((2R,3R,4R,5S)-3,4-dihydroxy-5-((6-(trifluoromethyl)pyrazin-2-yl)amino)tetrahydro-2H-pyran-2-yl)methyl)benzenesulfonamide (A112)

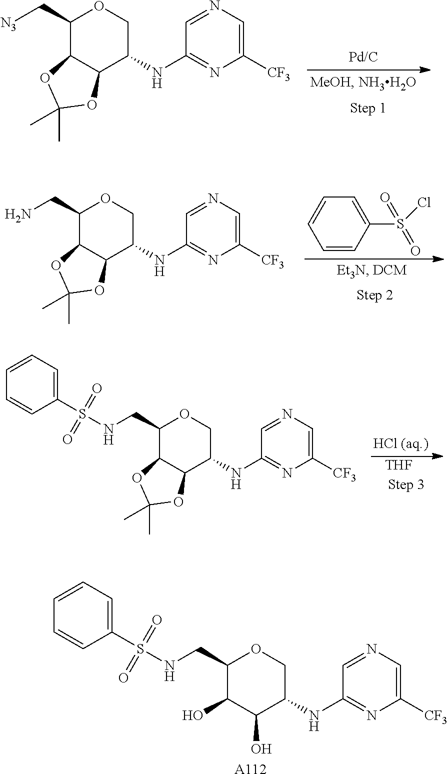

Step 1: To a solution of N-((3aS,4R,7S,7aR)-4-(azidomethyl)-2,2-dimethyltetrahydro-4H-[1,3]dioxolo[4,5-c]pyran-7-yl)-6-(trifluoromethyl)pyrazin-2-amine (1.00 eq) and NH3·H2O (1.00 eq) in MeOH (5 mL) was added Pd/C at 25° C. The mixture was degassed and purged with H2 several times, then stirred at 25° C. for 1 hour under H2 atmosphere. Then the mixture was filtered and the filtrate was concentrated to give N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine as a colorless oil. LCMS found: [M+H]+=349.
Step 2: The mixture of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.00 eq), benzenesulfonyl chloride (1.2 eq) and TEA (3.00 eq) in DCM (5 mL) was stirred at 25° C. for 3 hours. The mixture was quenched with water and extracted with DCM, the organic layer was dried over anhydrous sodium sulfate, filtered and concentrated. The residue was purified by silica gel chromatography (20-50% ethyl acetate in petroleum ether) to afford N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)benzenesulfonamide as a yellow oil. LCMS found: [M+H]⁺=489.
Step 3: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)benzenesulfonamide (1.00 eq) in THF (5 mL) was added HCl (1 mL, 2 N) at 0° C. The mixture was stirred at 25° C. for 3 hours. The mixture was concentrated and the residue was purified by reverse phase (C18, 0-40% MeCN in water, 0.3% TFA) to afford 112 as a colorless solid. LCMS found: [M+H]⁺=449. ¹H NMR (400 MHz, CD₃OD): δ 8.08 (s, 1H), 7.99 (s, 1H), 7.90-7.84 (m, 2H), 7.65-7.53 (m, 3H), 4.25 (td, J=10.8, 5.2 Hz, 1H), 4.03 (dd, J=11.2, 5.2 Hz, 1H), 3.83 (d, J=3.2 Hz, 1H), 3.55 (dd, J=10.8, 3.2 Hz, 1H), 3.39 (dd, J=8.0, 5.2 Hz, 1H), 3.16-3.04 (m, 2H), 2.94 (t, J=10.8 Hz, 1H).

### Preparation of 4-(3-(((2R,3R,4R,5S)-3,4-dihydroxy-5-((6-(trifluoromethyl)pyrazin-2-yl)amino)tetrahydro-2H-pyran-2-yl)methyl)ureido)benzoic acid (A113)

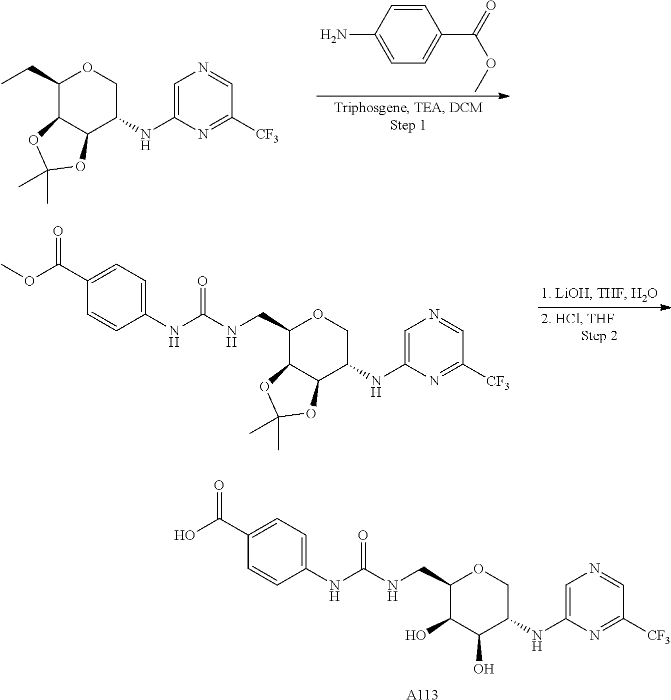

Step 1: To a solution of methyl 4-aminobenzoate (1.00 eq) in THF (10 mL) was added triphosgene (0.35 eq) at 0° C., the mixture was stirred at 25° C. for 0.5 h, then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.00 eq) was added. The mixture was stirred at 25° C. for 3 hours. The mixture was concentrated under reduced pressure, the residue was diluted with water, extracted with ethyl acetate, the combined organic layers were washed with brine, dried over anhydrous sodium sulfate, filtered and concentrated under reduced pressure. The residue was purified by silica gel chromatography (10-20% petroleum ether in ethyl acetate) to afford methyl 4-(3-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)ureido)benzoate as a white solid. LCMS found: [M+H]⁺=526.
Step 2: To a solution of methyl 4-(3-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)ureido)benzoate (1.00 eq) in THF (10 mL) and water (2 mL) was added LiOH (5.00 eq) at 0° C. for 5 hours, LCMS showed the starting material was consumed completely and HCl (15 mL, 2 N) was added to the reaction. The mixture was concentrated under reduced pressure, the residue was purified by prep-HPLC to afford A113 as a white solid. LCMS found: [M+H]⁻=470. ¹H NMR (400 MHz, CD₃OD): δ 8.11 (s, 1H), 8.00 (s, 1H), 7.91 (d, J=8.8 Hz, 2H), 7.47 (d, J=8.8 Hz, 2H), 4.35 (td, J=10.6, 5.1 Hz, 1H), 4.15 (dd, J=11.2, 5.2 Hz, 1H), 3.89 (d, J=3.2 Hz, 1H), 3.66 (dd, J=10.8, 3.2 Hz, 1H), 3.59-3.51 (m, 2H), 3.39-3.34 (m, 1H), 3.12 (t, J=10.8 Hz, 1H).

### Preparation of 1-(((2R,3R,4R,5S)-3,4-dihydroxy-5-((6-(trifluoromethyl)pyrazin-2-yl)amino)tetrahydro-2H-pyran-2-yl)methyl)-3-phenylurea (A114)

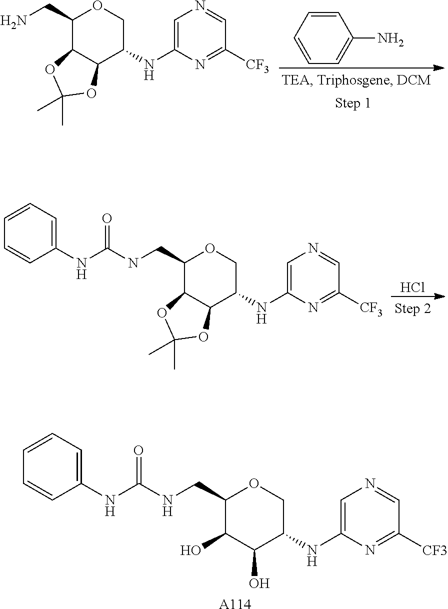

Step 1: To a solution of aniline (1.50 eq) in DCM (10 mL) was added triphosgene (0.90 eq) in DCM (8 mL). The mixture was stirred at 25° C. for 30 mins, then concentrated. The residue was dissolved in DCM (10 mL), then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.00 eq) in DCM (10 mL) was added at 25° C. The mixture was stirred at 25° C. for 2 hours, then extracted with EA and H₂O. The organic layers were concentrated and the residue was purified by silica gel column chromatography (5% ethyl acetate in petroleum ether) to afford 1-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-3-phenylurea as a white solid. LCMS found: [M+H]⁺=468.
Step 2: To a solution of 1-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-3-phenylurea (1.00 eq) in THF (10 mL) was added HCl (1 mL, 2 N) at 25° C. The mixture was stirred at 25° C. for 2 hours, then concentrated. The residue was purified by prep-HPLC to afford A114 as a white solid. LCMS found: [M+H]⁺=428. 1H NMR (400 MHz, DMSO-d₆): δ 8.58 (s, 1H), 8.22 (s, 1H), 8.08 (s, 1H) 7.65 (d, J=7.6 Hz, 1H), 7.38 (d, J=7.6 Hz, 2H), 7.22 (t, J=8.0 Hz, 2H), 6.89 (t, J=7.2 Hz, 1H) 6.21 (t, J=5.6 Hz, 1H), 4.88 (t, J=5.6 Hz, 2H), 4.13-4.04 (m, 1H), 3.93 (dd, J=10.8, 5.2 Hz, 1H), 3.71 (t, J=4.0 Hz, 1H), 3.58-3.50 (m, 1H), 3.26-3.17 (m, 1H), 2.98 (t, J=10.8 Hz, 1H).

### Preparation of 6-(3-(((2R,3R,4R,5S)-3,4-dihydroxy-5-((6-(trifluoromethyl)pyrazin-2-yl)amino)tetrahydro-2H-pyran-2-yl)methyl)ureido)hexanoic acid (A115)

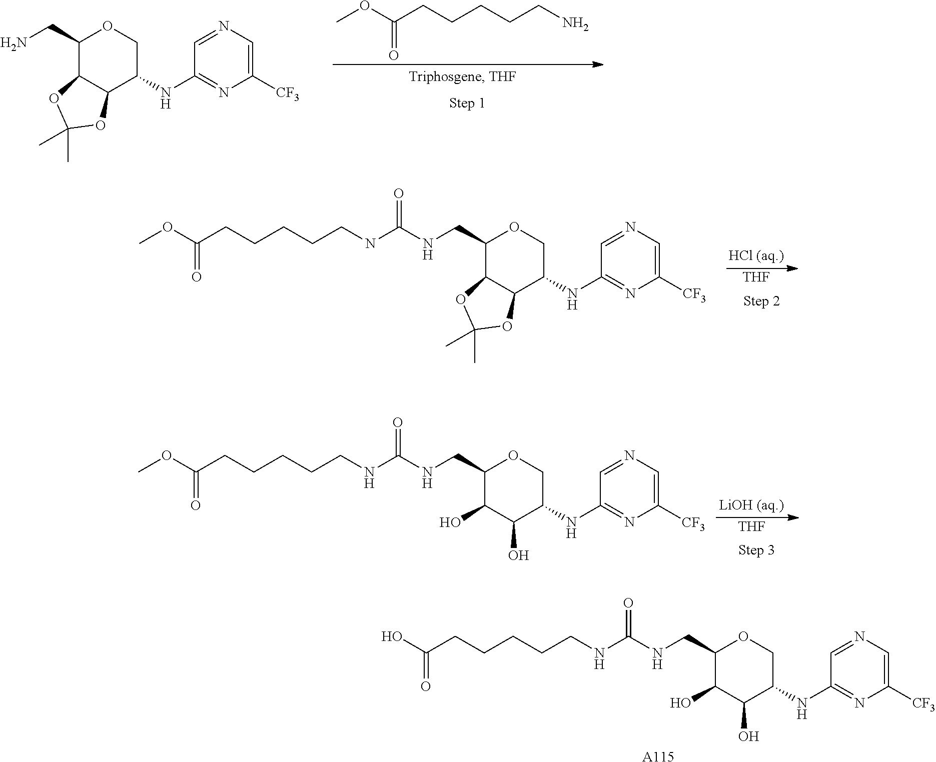

Step 1: To a solution of methyl 6-aminohexanoate (1.00 eq) in DCM (10 mL) was added triphosgene (0.4 eq) at 0° C., the mixture was stirred for 0.56 h, then N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.00 eq) was added. The mixture was stirred at 25° C. for 3 hours. TLC showed one major spot was detected. The mixture was concentrated under reduced pressure, the residue was diluted with water, extracted with ethyl acetate, the combined organic layers were washed with saturated solution of sodium bicarbonate, dried over anhydrous sodium sulfate, filtered and concentrated under reduced pressure. The residue was purified by silica gel chromatography (10-20% petroleum ether in ethyl acetate) to afford methyl 6-(3-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)ureido)hexanoate as a white solid. LCMS found: [M+H]⁺=520.
Step 2: To a solution of methyl 6-(3-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)ureido)hexanoate (1.00 eq) in THF (10 mL) was added HCl (2 mL, 2N), the mixture was stirred at 25° C. for 4 hours. The mixture was concentrated under reduced pressure to afford methyl 6-(3-(((2R,3R,4R,5S)-3,4-dihydroxy-5-((6-(trifluoromethyl)pyrazin-2-yl)amino)tetrahydro-2H-pyran-2-yl)methyl)ureido)hexanoate (crude) as a colorless oil. LCMS found: [M+H]⁺=480.
Step 3: To a solution of methyl 6-(3-(((2R,3R,4R,5S)-3,4-dihydroxy-5-((6-(trifluoromethyl)pyrazin-2-yl)amino)tetrahydro-2H-pyran-2-yl)methyl)ureido)hexanoate (1.00 eq) in THF (5 mL) and H₂O (1 mL) was added LiOH (5.00 eq), the mixture was stirred at 25° C. for 4 hours. The mixture was concentrated under reduced pressure, the residue was purified by prep-HPLC to afford A115 as a colorless oil. LCMS found: [M+H]⁺=466. ¹H NMR (400 MHz, CD₃OD): δ 8.10 (s, 1H), 8.00 (s, 1H), 4.32 (td, J=10.8, 5.2 Hz, 1H), 4.12 (dd, J=11.2, 5.2 Hz, 1H), 3.84 (d, J=3.2 Hz, 1H), 3.64 (dd, J=10.8, 3.2 Hz, 1H), 3.47-3.41 (m, 2H), 3.28-3.21 (m, 1H), 3.14-3.08 (m, 3H), 2.29 (t, J=7.2 Hz, 2H), 1.68-1.56 (m, 2H), 1.53-1.45 (m, 2H), 1.40-1.31 (m, 2H).

### Preparation of 2-(2-(2-((((2R,3R,4R,5S)-3,4-dihydroxy-5-((2-(trifluoromethyl)pyrimidin-4-yl)amino)tetrahydro-2H-pyran-2-yl)methyl)amino)-2-oxoethoxy)ethoxy)acetic acid (A116)

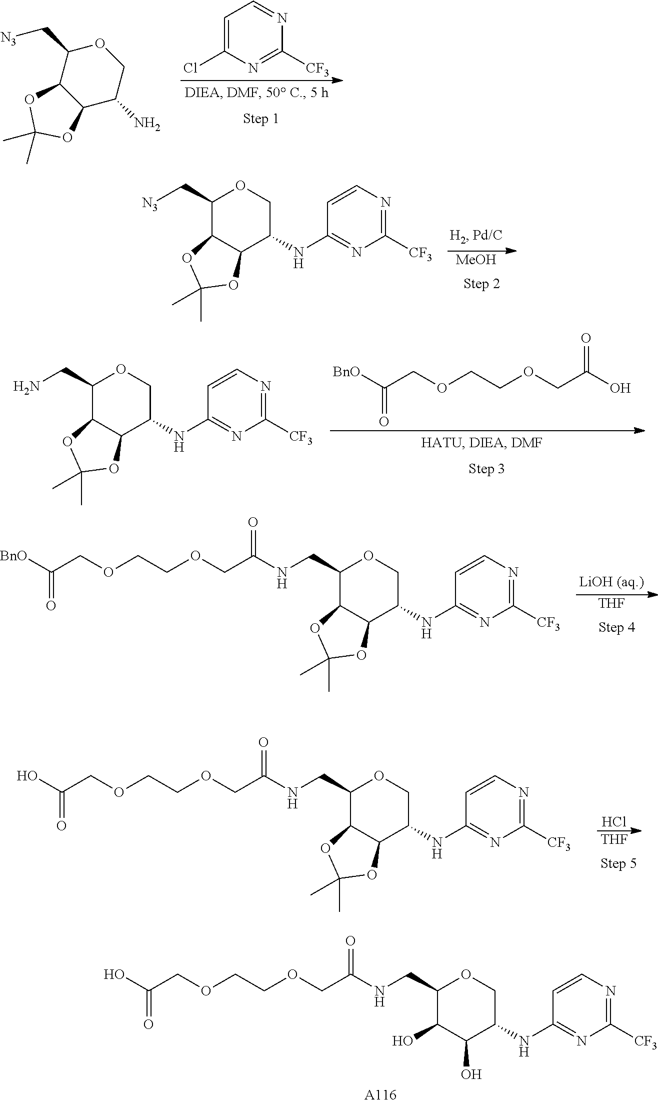

Step 1: To a solution of (3aS,4R,7S,7aR)-4-(azidomethyl)-2,2-dimethyltetrahydro-4H-[1,3]dioxolo[4,5-c]pyran-7-amine (1.00 eq) in DMF (10 mL) were added DIPEA (3.00 eq) and 4-chloro-2-(trifluoromethyl)pyrimidine (1.20 eq) at 25° C. The mixture was stirred at 50° C. for 5 hours, then concentrated under reduced pressure. The residue was purified by silica gel column chromatography (1-5% MeOH in DCM) to afford N-((3aS,4R,7S,7aR)-4-(azidomethyl)-2,2-dimethyltetrahydro-4H-[1,3]dioxolo[4,5-c]pyran-7-yl)-2-(trifluoromethyl)pyrimidin-4-amine as a white solid. LCMS found: [M+H]⁺=375.
Step 2: To a solution of N-((3aS,4R,7S,7aR)-4-(azidomethyl)-2,2-dimethyltetrahydro-4H-[1,3]dioxolo[4,5-c]pyran-7-yl)-2-(trifluoromethyl)pyrimidin-4-amine (1.00 eq) in MeOH (20 mL) was added Pd/C, the mixture was degassed and purged with H2 several times, then stirred at 25° C. for 1 hour under H2 atmosphere. The mixture was filtered and the filtrate was concentrated to give N-((3aS,4R,7S,7aR)-4-(aminomethyl)-2,2-dimethyltetrahydro-4H-[1,3]dioxolo[4,5-c]pyran-7-yl)-2-(trifluoromethyl)pyrimidin-4-amine as a colorless oil. LCMS found: [M+H]⁺=349.
Step 3: To a solution of 2-(2-(2-(benzyloxy)-2-oxoethoxy)ethoxy)acetic acid (1.20 eq) in DMF (10 mL) were added DIPEA (3.00 eq) and HATU (1.50 eq), the mixture was stirred at 0° C. for 0.5 h, then N-((3aS,4R,7S,7aR)-4-(aminomethyl)-2,2-dimethyltetrahydro-4H-[1,3]dioxolo[4,5-c]pyran-7-yl)-2-(trifluoromethyl)pyrimidin-4-amine (1.00 eq) was added at 25° C. and the resulting mixture was stirred at 25° C. for 3 hours. The mixture was diluted with water, extracted with ethyl acetate, the combined organic layers were washed with brine, dried over anhydrous sodium sulfate, filtered and concentrated under reduced pressure. The residue was purified by silica gel column chromatography (50% ethyl acetate in petroleum ether) to afford benzyl 2-(2-(2-((((3aS,4R,7S,7aR)-2,2-dimethyl-7-((2-(trifluoromethyl)pyrimidin-4-yl)amino)tetrahydro-4H-[1,3]dioxolo[4,5-c]pyran-4-yl)methyl)amino)-2-oxoethoxy)ethoxy)acetate as a yellow oil solid. LCMS found: [M+H]⁺=599.
Step 4: To a solution of benzyl 2-(2-(2-((((3aS,4R,7S,7aR)-2,2-dimethyl-7-((2-(trifluoromethyl)pyrimidin-4-yl)amino)tetrahydro-4H-[1,3]dioxolo[4,5-c]pyran-4-yl)methyl)amino)-2-oxoethoxy)ethoxy)acetate (1.00 eq) in THF (10 mL) was added LiOH (5.00 eq) in H₂O (2 mL) at 0° C. The mixture was stirred at 25° C. for 2 hours, then concentrated under reduced pressure to afford 2-(2-(2-((((3aS,4R,7S,7aR)-2,2-dimethyl-7-((2-(trifluoromethyl)pyrimidin-4-yl)amino)tetrahydro-4H-[1,3]dioxolo[4,5-c]pyran-4-yl)methyl)amino)-2-oxoethoxy)ethoxy)acetic acid as a white solid. LCMS found: [M+H]⁺=509.
Step 5: To a solution of 2-(2-(2-((((3aS,4R,7S,7aR)-2,2-dimethyl-7-((2-(trifluoromethyl)pyrimidin-4-yl)amino)tetrahydro-4H-[1,3]dioxolo[4,5-c]pyran-4-yl)methyl)amino)-2-oxoethoxy)ethoxy)acetic acid (1.00 eq) in THF (10 mL) was added HCl (1 mL, 2 N). The mixture was stirred at 25° C. for 2 hours, then concentrated. The residue was purified by prep-HPLC to afford compound A116 as a colorless oil. LCMS found: [M+H]⁺=469. ¹H NMR (400 MHz, CD₃OD): δ 8.16-8.02 (m, 1H), 6.84-6.56 (m, 1H), 4.54-4.39 (m, 1H), 4.13-4.03 (m, 1H), 4.00 (s, 2H), 3.92 (s, 2H), 3.87 (d, J=3.2 Hz, 1H), 3.72-3.65 (m, 4H), 3.62 (dd, J=10.4, 3.2 Hz, 1H), 3.58-3.45 (m, 3H), 3.17-3.06 (m, 1H).

### Preparation of (2R,5S)-2-(((3-methyloxetan-3-yl)amino)methyl)-5-((6-(trifluoromethyl)pyrazin-2-yl)amino)tetrahydro-2H-pyran-3,4-diol (A119)

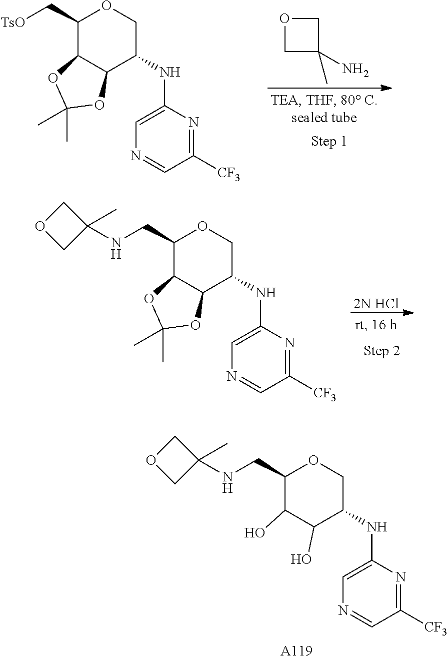

Step 1: To a solution of ((3aR,4R,7S,7aR)-2,2-dimethyl-7-((6-(trifluoromethyl) pyrazin-2-yl)amino)tetrahydro-4H-[1,3]dioxolo[4,5-c]pyran-4-yl)methyl 4-methyl benzenesulfonate (50 mg, 0.10 mmol) in THF (1 mL) were added 3-methyloxetan-3-amine (0.013 mL, 0.30 mmol) and TEA (30 mg, 0.30 mmol) at an ambient temperature. The mixture was stirred at 80° C. for 3 days. The mixture was concentrated and the residue was purified by prep-TLC to give N-((4R,7S)-2,2-dimethyl-4-(((3-methyloxetan-3-yl)amino) methyl)tetrahydro-4H-[1,3]dioxolo[4,5-c]pyran-7-yl) 6-(trifluoromethyl)pyrazin-2-amine (20 mg, 48% yield) as yellow oil. LC-MS (ESI) found: 419 [M+H]⁺.
Step 2: To a solution of N-((4R,7S)-2,2-dimethyl-4-(((3-methyloxetan-3-yl)amino)methyl)tetrahydro-4H-[1,3]dioxolo[4,5-c]pyran-7-yl)-6-(trifluoromethyl) pyrazin-2-amine (20 mg, 0.048 mmol) in THF (1 mL) was added HCl (1 mL, 2 N in H₂O). The mixture was stirred at an ambient temperature overnight. The mixture was concentrated in vacuo. The crude product was purified by prep-HPLC to give (2R,5S)-2-(((3-methyloxetan-3-yl) amino)methyl)-5-((6-(trifluoromethyl) pyrazin-2-yl)amino)tetrahydro-2H-pyran-3,4-diol (1.2 mg, 7% yield) as white solid. LC-MS (ESI) found: 379 [M+H]⁺. ¹H NMR (400 MHz, CD₃OD): δ 8.12 (s, 1H), 8.02 (s, 1H), 4.71 (d, J=7.0 Hz, 2H), 4.47 (dd, J=7.0, 3.3 Hz, 2H), 4.36 (d, J=4.9 Hz, 1H), 4.20 (dd, J=11.0, 5.0 Hz, 1H), 3.94 (d, J=2.6 Hz, 1H), 3.77-3.62 (m, 2H), 3.20 (dd, J=11.5, 9.1 Hz, 2H), 3.07 (dd, J=12.6, 3.2 Hz, 1H), 1.61 (s, 3H).

### Preparation of N-(((2R,3R,4R,5S)-3,4-dihydroxy-5-((6-(trifluoromethyl)pyrazin-2-yl)amino)tetrahydro-2H-pyran-2-yl)methyl)pyridazine-3-carboxamide (A120)

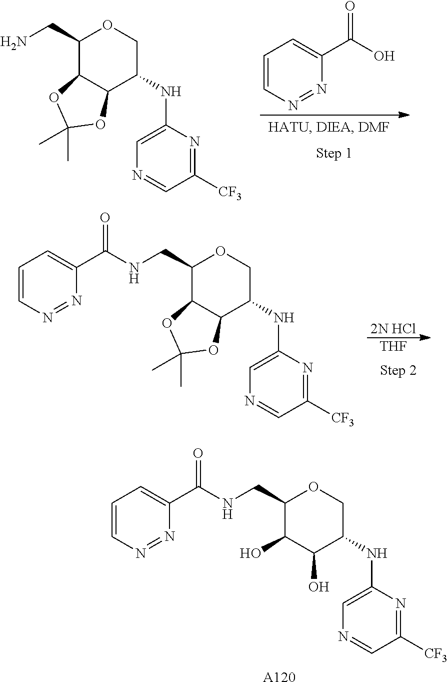

Step 1: A solution of pyridazine-3-carboxylic acid (35 mg, 0.28 mmol), N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (98.2 mg, 0.28 mmol), DIEA (0.09 mL, 0.56 mmol) and HATU (128.7 mg, 0.34 mmol) in DMF (1 mL) was stirred at 90° C. overnight. The mixture was directly purified by C18 column to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazine-3-carboxamide (80 mg, 62% yield) as a yellow solid. LC-MS (ESI) found: 455 [M+H]⁺. ¹H NMR (400 MHz, CD₃OD): δ 9.33 (d, J=4.5 Hz, 1H), 8.32 (d, J=8.5 Hz, 1H) 8.09 (s, 1H), 8.02 (s, 1H), 7.90 (dd, J=8.4, 5.1 Hz, 1H), 4.33 (dd, J=14.7, 4.1 Hz, 2H), 4.17 (dd, J=8.7, 5.1 Hz, 1H), 4.05-3.89 (m, 3H), 3.75 (dd, J=14.0, 8.1 Hz, 1H), 3.16 (t, J=11.2 Hz, 1H), 1.55 (s, 3H), 1.37 (s, 3H). ¹⁹F NMR (377 MHz, CD₃OD): δ −70.37 (s).
Step 2: A solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyridazine-3-carboxamide (80 mg, 0.18 mmol) in THF (1 mL) was added HCl (1 mL, 2 N THF). The reaction was stirred at an ambient temperature for 2 h. The resulting mixture was filtered and concentrated in vacuo. The crude product was purified by prep-HPLC to give N-(((2R,3R,4R,5S)-3,4-dihydroxy-5-((6-(trifluoromethyl)pyrazin-2-yl)amino)tetrahydro-2H-pyran-2-yl)methyl)pyridazine-3-carboxamide (58 mg, 79% yield) as a white solid. LC-MS (ESI) found: 415 [M+H]⁺. ¹H NMR (400 MHz, CD₃OD): δ 9.32 (d, J=4.6 Hz, 1H), 8.31 (d, J=8.4 Hz, 1H), 8.11 (s, 1H), 8.00 (s, 1H), 7.90 (dd, J=8.4, 5.1 Hz, 1H), 4.37 (td, J=10.4, 5.1 Hz, 1H), 4.15 (dd, J=11.0, 5.1 Hz, 1H) 3.94 (d, J=2.5 Hz, 1H), 3.79 (t, J=9.0 Hz, 1H), 3.73-3.66 (m, 3H), 3.14 (t, J=10.8 Hz, 1H). ¹⁹F NMR (377 MHz, CD₃OD): δ −70.31 (s).

### Preparation of (2R,3R,4R,5S)-2-(((3-hydroxyphenyl)(methyl)amino)methyl)-5-((6-(trifluoromethyl)pyrazin-2-yl)amino)tetrahydro-2H-pyran-3,4-diol (A121)

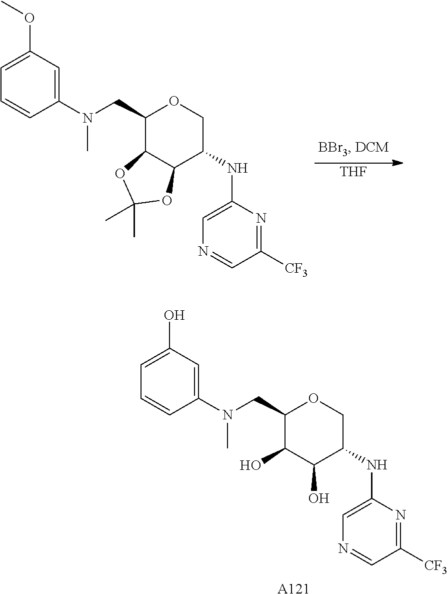 [SMILES: COc1cccc(N(C)C[C@H]2OC[C@H](Nc3cncc(C(F)(F)F)n3)[C@H]3OC(C)(C)O[C@H]32)c1 ; CN(C[C@H]1OC[C@H](Nc2cncc(C(F)(F)F)n2)[C@@H](O)[C@H]1O)c1cccc(O)c1 ; "A121" ; "THF"]

To a solution of N-((3aS,4R,7S,7aR)-4-(((3-methoxyphenyl)(methyl)amino)methyl)-2,2-dimethyltetrahydro-4H-[1,3]dioxolo[4,5-c]pyran-7-yl)-6-(trifluoromethyl)pyrazin-2-amine (20 mg, 0.04 mmol) in DCM (2 mL) was added BBr₃ (3 mL) in an ice bath. The reaction mixture was stirred at an ambient temperature overnight. Reaction mixture was quenched with MeOH and concentrated. The residue was purified by C18 column (MeOH in water from 5% to 70%) to give (2R,3R,4R,5S)-2-(((3-hydroxyphenyl)(methyl)amino)methyl)-5-((6-(trifluoromethyl)pyrazin-2-yl)amino)tetrahydro-2H-pyran-3,4-diol as a white solid (2.7 mg, 11% yield). LC-MS (ESI) found: 415 [M+H]⁺. ¹H NMR (400 MHz, CD₃OD): δ 8.09 (s, 1H), 7.99 (s, 1H), 6.98 (t, J=8.0 Hz, 1H) 6.26 (d, J=8.2 Hz, 1H), 6.21 (s, 1H), 6.13 (d, J=7.9 Hz, 1H), 4.36 (td, J=10.6, 5.1 Hz, 1H) 4.12 (dd, J=10.9, 5.1 Hz, 1H), 3.85 (s, 1H), 3.68-3.47 (m, 4H), 3.06 (t, J=10.8 Hz, 1H), 2.96 (s, 3H).

### Preparation of N-(((2R,3R,4R,5S)-3,4-dihydroxy-5-((6-(trifluoromethyl)pyrazin-2-yl)amino)tetrahydro-2H-pyran-2-yl)methyl)-3-fluoro-1H-pyrazole-5-carboxamide (A122)

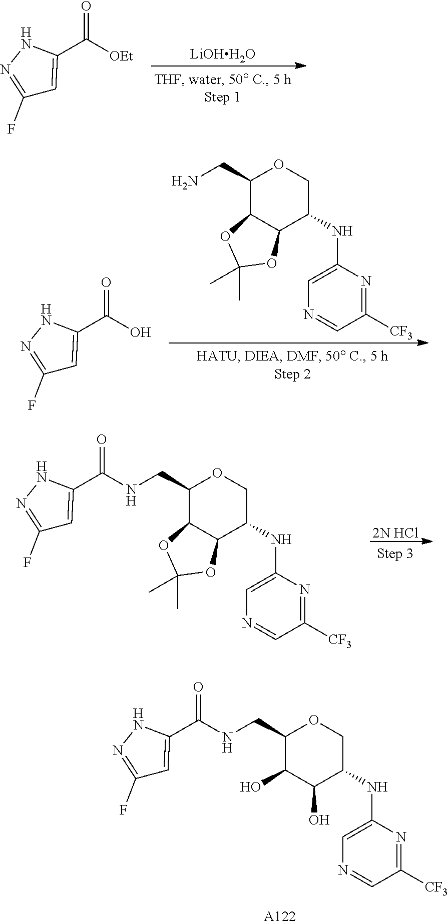

Step 1: To a solution of ethyl 3-fluoro-1H-pyrazole-5-carboxylate (40 mg, 0.25 mmol) in THF (3 mL) was LiOH·H₂O (17.5 mg, 0.76 mmol) and water (0.5 mL) at an ambient temperature. The reaction was stirred at 50° C. for 5 h. TLC (petroleum ether/ethyl acetate=3/1) showed starting material was consumed and one main spot detected. The reaction was added HCl solution (5 mL, 2N in H₂O), extracted with EA (20 mL×3), washed with brine, dried, concentrated to give crude 3-fluoro-1H-pyrazole-5-carboxylic acid (25 mg, 76% yield) as a white solid. LC-MS (ESI) found: 129 [M−H]⁻.
Step 2: To a solution of 3-fluoro-1H-pyrazole-5-carboxylic acid (25 mg, 0.194 mmol) in DMF (3 mL) was added N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (73.6 mmol, 0.211 mmol), HATU (110 mg, 0.291 mmol) and DIPEA (1 mL) at an ambient temperature. The reaction was heated to 50° C. for 16 h. TLC showed starting material was consumed and one main spot detected. The reaction was concentrated and purified by silica gel with petroleum ether/ethyl acetate=1/1 to give N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-3-fluoro-1H-pyrazole-5-carboxamide (40 mg, 46% yield) as yellow solid. LC-MS (ESI) found: 451 [M+H]⁺.
Step 3: To a solution of N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)-3-fluoro-1H-pyrazole-5-carboxamide in THF (2 mL) was added HCl solution (1 mL, 2N in H₂O) at an ambient temperature. The reaction was stirred at an ambient temperature for 30 min. LC-MS showed staring material was consumed and desired mass was detected. Then the mixture was concentrated under reduced pressure to give N-(((2R,3R,4R,5S)-3,4-dihydroxy-5-((6-(trifluoromethyl)pyrazin-2-yl)amino)tetrahydro-2H-pyran-2-yl)methyl)-3-fluoro-1H-pyrazole-5-carboxamide (35 mg, 93% yield) as a yellow solid. LC-MS (ESI) found: 421 [M+H]⁺. ¹H NMR (400 MHz, DMSO-d₆): δ 13.21 (s, 1H), 8.64 (t, J=5.6 Hz, 1H), 8.22 (s, 1H), 8.06 (s, 1H), 7.65 (d, J=6.8 Hz, 1H), 6.58 (d, J=6.4 Hz, 1H), 3.92 (dd, J=10.8 Hz, 2H), 3.74 (d, J=2.8 Hz, 1H), 3.58-3.47 (m, 2H), 3.42 (t, J=5.6 Hz, 2H), 2.97 (t, J=10.8 Hz, 1H).

### Preparation of (2R,3R,4R,5S)-2-((pyrazin-2-ylamino)methyl)-5-((6-(trifluoromethyl)pyrazin-2-yl)amino)tetrahydro-2H-pyran-3,4-diol (A123)

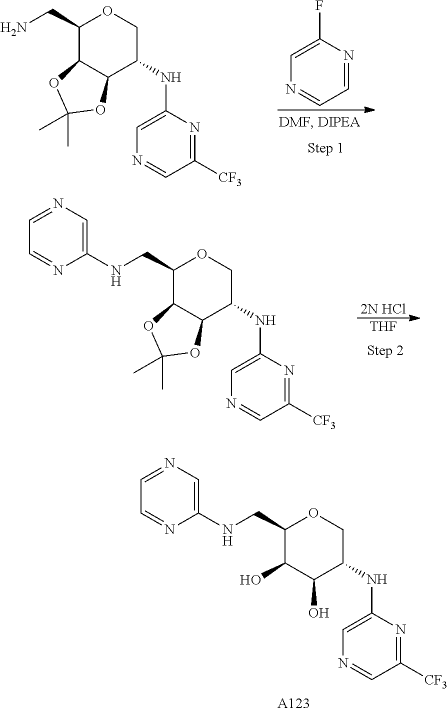

Step 1: A solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (50 mg, 0.14 mmol), 2-fluoropyrazine (21 mg, 0.21 mmol) and DIPEA (37 mg, 0.28 mmol) in DMF (2 mL) was stirred at 100° C. overnight. The mixture was concentrated and the residue was purified by flash to give N-((3aS,4R,7S,7aR)-2,2-dimethyl-4-((pyrazin-2-ylamino)methyl)tetrahydro-4H-[1,3]dioxolo[4,5-c]pyran-7-yl)-6-(trifluoromethyl)pyrazin-2-amine (30 mg, 49% yield) as yellow solid. LC-MS (ESI) found: 427 [M+H]⁺.
Step 2: To a solution of N-((3aS,4R,7S,7aR)-2,2-dimethyl-4-((pyrazin-2-ylamino)methyl)tetrahydro-4H-[1,3]dioxolo[4,5-c]pyran-7-yl)-6-(trifluoromethyl)pyrazin-2-amine (30 mg, 0.07 mmol) in THF (3 mL) was added HCl solution (0.5 mL, 2 N in H₂O). The mixture was stirred at an ambient temperature overnight, then concentrated. The residue was purified by flash chromatography to give (2R,3R,4R,5S)-2-((pyrazin-2-ylamino)methyl)-5-((6-(trifluoromethyl)pyrazin-2-yl)amino)tetrahydro-2H-pyran-3,4-diol (14.5 mg, 53% yield) as yellow solid. LC-MS (ESI) found: 387 [M+H]⁺. ¹H NMR (400 MHz, CD₃OD): δ 8.10 (s, 1H) 8.00 (s, 1H), 7.93 (d, J=5.2 Hz, 2H), 7.64 (d, J=2.9 Hz, 1H), 4.34 (s, 1H), 4.14 (dd, J=11.0, 5.3 Hz, 1H), 3.89 (d, J=2.8 Hz, 1H), 3.69-3.58 (m, 3H), 3.54-3.48 (m, 1H), 3.11 (t, J=10.9 Hz, 1H).

### Preparation of tert-butyl (6-((((2R,3R,4R,5S)-3,4-dihydroxy-5-((6-(trifluoromethyl)pyrazin-2-yl)amino)tetrahydro-2H-pyran-2-yl)methyl)amino)spiro[3.3]heptan-2-yl)carbamate (A124)

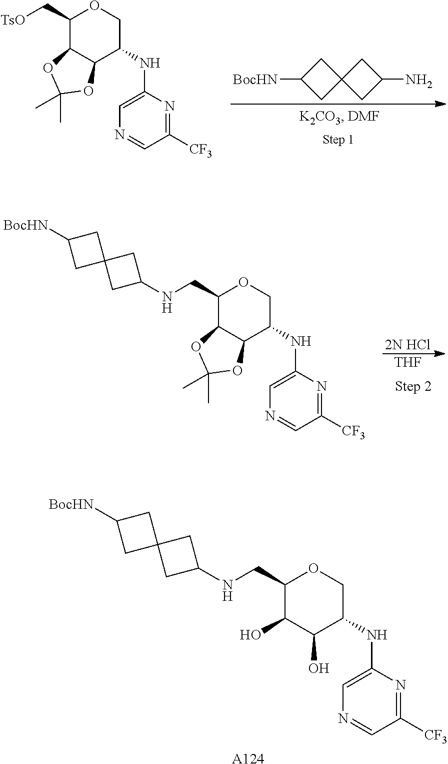

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (30 mg, 0.06 mmol) and tert-butyl (6-aminospiro[3.3]heptan-2-yl)carbamate (27 mg, 0.12 mmol) in DMF (2 mL) was added K₂CO₃ (24.7 mg, 0.18 mmol) at an ambient temperature under N₂. The reaction was stirred at 100° C. overnight. The resulting mixture was concentrated in vacuo. The crude product was purified by flash chromatography (silica gel, 0-15% MeOH in DCM) to give tert-butyl (6-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)spiro[3.3]heptan-2-yl)carbamate (12 mg, 36% yield) as a colorless oil. LC-MS (ESI) found: 558 [M+H]⁺.
Step 2: To a solution of tert-butyl (6-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)spiro[3.3]heptan-2-yl)carbamate (12 mg, 0.022 mmol) in THF (5 mL) was added HCl solution (1 mL, 2 N in H₂O). The reaction was stirred at an ambient temperature overnight. The resulting mixture was concentrated in vacuo. The crude product was purified by prep-HPLC to give tert-butyl (6-((((2R,3R,4R,5S)-3,4-dihydroxy-5-((6-(trifluoromethyl)pyrazin-2-yl)amino)tetrahydro-2H-pyran-2-yl)methyl)amino)spiro[3.3]heptan-2-yl)carbamate (0.8 mg, 7% yield) as a white solid. LC-MS (ESI) found: 518 [M+H]⁺. ¹H NMR (400 MHz, CD₃OD): δ 8.11 (s, 1H), 8.00 (s, 1H), 4.32 (td, J=10.5, 5.2 Hz, 1H), 4.13 (dd, J=11.0, 5.1 Hz, 1H), 3.93-3.81 (m, 2H), 3.64 (dd, J=10.6, 3.2 Hz, 1H), 3.49 (d, J=3.7 Hz, 1H), 3.24-3.15 (m, 1H), 3.11 (t, J=10.9 Hz, 1H), 2.95-2.84 (m, 1H), 2.65 (dd, J=12.6, 3.7 Hz, 1H), 2.37 (d, J=11.3 Hz, 2H), 2.24-2.18 (m, 2H), 1.94-1.79 (m, 4H), 1.42 (s, 9H). ¹⁹F NMR (377 MHz, CD₃OD): δ −70.34 (s).

### Preparation of 3-(4-(2-((((2R,3R,4R,5S)-3,4-dihydroxy-5-((6-(trifluoromethyl)pyrazin-2-yl)amino)tetrahydro-2H-pyran-2-yl)methyl)amino)-2-oxoethyl)piperazin-1-yl)propanoic acid (A125)

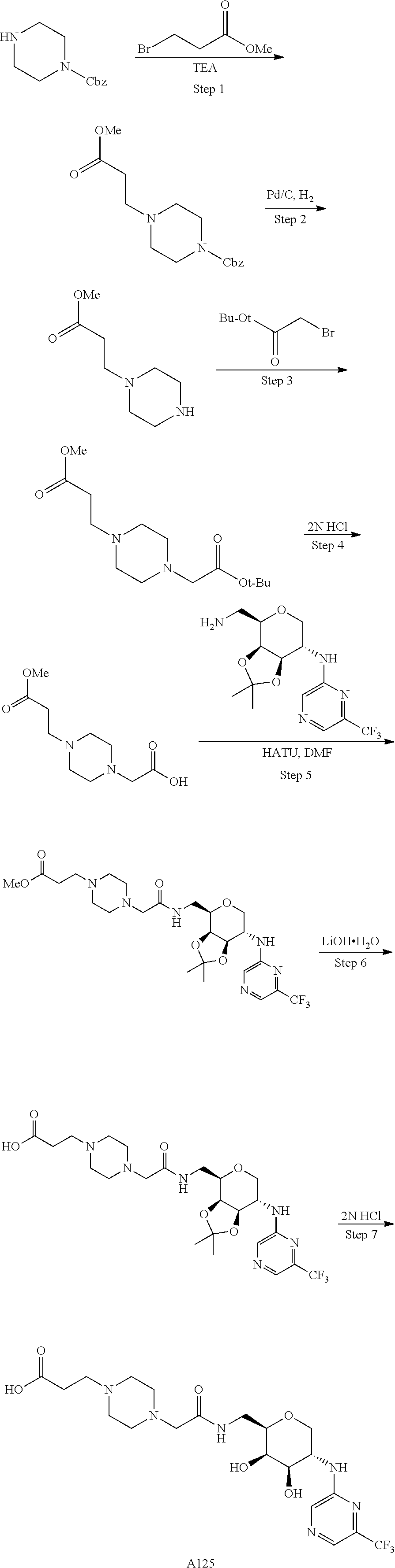

Step 1: To a solution of benzyl piperazine-1-carboxylate (22 g, 99.8 mmol) and methyl 3-bromopropanoate (16.7 g, 99.8 mmol) in DCM (150 mL) was added TEA (20.3 g, 299.6 mmol) at an ambient temperature. The reaction solution was stirred at an ambient temperature for 18 h. The reaction was concentrated and purified by silica gel with PE/EA=2/1 to give benzyl 4-(3-methoxy-3-oxopropyl)piperazine-1-carboxylate (25 g, 82% yield) as colorless oil. LC-MS (ESI) found: 307 [M+H]⁺. ¹H NMR (400 MHz, DMSO-d₆): δ 7.29-7.38 (m, 5H), 5.13 (s, 2H), 3.68 (s, 3H), 3.50 (t, J=4.8 Hz, 4H), 2.70 (t, J=7.2 Hz, 2H), 2.50 (d, J=7.2 Hz, 2H), 2.36-2.47 (m, 4H).
Step 2: To a solution of benzyl 4-(3-methoxy-3-oxopropyl)piperazine-1-carboxylate (4 g, 13.1 mmol) in MeOH (30 mL) was added Pd/C (400 mg, 10% wt., wet 60%) under a H2 balloon at an ambient temperature. The reaction was stirred at an ambient temperature for 18 h. The reaction was filtered and the residue was concentrated to give methyl 3-(piperazin-1-yl)propanoate (4 g, 89% yield) as colorless oil. LC-MS (ESI) found: 173 [M+H]⁺.
Step 3: To a solution of methyl 3-(piperazin-1-yl)propanoate (4 g, 23.2 mmol) in DCM (50 mL) was added Et₃N (7.0 g, 69.6 mmol) and tert-butyl 2-bromoacetate (8.97 g, 46.4 mmol) at an ambient temperature. Then the reaction was stirred at an ambient temperature for 18 h. Then the reaction was concentrated and purified by silica gel with petroleum ether/ethyl acetate=1/1 to ethyl acetate to give methyl 3-(4-(2-(tert-butoxy)-2-oxoethyl)piperazin-1-yl)propanoate (5 g, 53% yield) as yellow solid. LC-MS (ESI) found: 287 [M+H]⁺. ¹H NMR (400 MHz, CD₃OD): δ 3.67 (s, 3H), 3.13 (s, 2H), 2.72 (d, J=7.2 Hz, 2H), 2.48-2.66 (m, 10H), 1.47 (s, 9H).
Step 4: To a solution of methyl 3-(4-(2-(tert-butoxy)-2-oxoethyl)piperazin-1-yl)propanoate (2 g, 7.0 mmol) in DCM (20 mL) was added HCl-dioxane solution (10 mL, 2 M) at an ambient temperature. The reaction was stirred at 50° C. for 18 h. Then the reaction mixture was concentrated to give crude 2-(4-(3-methoxy-3-oxopropyl)piperazin-1-yl)acetic acid (1.3 g, 80% yield). LC-MS (ESI) found: 231 [M+H]⁺. ¹H NMR (400 MHz, DMSO-d₆): δ 4.11 (s, 2H), 3.65 (s, 3H), 3.45-3.61 (m, 8H), 3.39 (t, J=7.6 Hz, 2H), 2.95 (t, J=7.6 Hz, 2H).
Step 5: To a solution of 2-(4-(3-methoxy-3-oxopropyl)piperazin-1-yl)acetic acid (687.5 mg, 2.9 mmol) in DMF (15 mL) was added DIEA (890 mg, 6.9 mmol) and HATU (1.3 g, 3.5 mmol) at an ambient temperature. The solution was stirred at an ambient temperature for 10 min. Then N-((3aS,4R,7S,7aR)-4-(aminomethyl)-2,2-dimethyltetrahydro-4H-[1,3]dioxolo[4,5-c]pyran-7-yl) 6-(trifluoromethyl)pyrazin-2-amine (800 mg, 2.3 mmol) in DMF (5 mL) was added at an ambient temperature. Then the reaction was stirred at an ambient temperature for 18 h. LC-MS showed 30% desired product. Then the reaction was concentrated and purified by silica gel with DCM/MeOH=4/1 to give methyl 3-(4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)piperazin-1-yl)propanoate (170 mg, 13% yield) as yellow solid. LC-MS (ESI) found: 560.8 [M+H]⁺. ¹H NMR (400 MHz, CD₃OD): δ 8.09 (s, 1H), 8.02 (s, 1H), 4.24-4.32 (m, 2H), 4.14 (dd, J=8.8, 4.8 Hz, 1H), 3.95 (dd, J=11.2, 5.2 Hz, 1H), 3.86-3.89 (m, 1H) 3.63-3.72 (m, 3H), 3.43-3.50 (m, 1H), 3.06-3.19 (m, 4H), 2.75-2.79 (m, 2H), 2.52-2.70 (m, 8H) 1.53 (s, 3H), 1.35 (s, 3H).
Step 6: To a solution of methyl 3-(4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)piperazin-1-yl)propanoate (170 mg, 0.3 mmol) in THF (4 mL) and H₂O (2 mL) was added LiOH·H₂O (38.2 mg, 0.9 mmol) at an ambient temperature. The reaction was stirred at an ambient temperature for 18 h. The reaction was concentrated to give crude 3-(4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)piperazin-1-yl)propanoic acid (165 mg) which was used for next step directly. LC-MS (ESI) found: 546.8 [M+H]⁺.
Step 7: To a solution of 3-(4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)piperazin-1-yl)propanoic acid (165 mg, 0.302 mmol) in THF (2 mL) was added HCl solution (1 mL, 2 N in H₂O) and the mixture was stirred at an ambient temperature for 18 h. Then the reaction was concentrated and purified by reverse phase with H₂O (0.5% NH₄HCO3)/CH₃CN to give 3-(4-(2-((((2R,3R,4R,5S)-3,4-dihydroxy-5-((6-(trifluoromethyl)pyrazin-2-yl)amino)tetrahydro-2H-pyran-2-yl)methyl)amino)-2-oxoethyl)piperazin-1-yl)propanoic acid (94 mg, 62% yield) as white solid. LC-MS (ESI) found: 506.9 [M+H]⁺. ¹H NMR (400 MHz, CD₃OD): δ 8.13 (s, 1H), 8.00 (s, 1H), 4.30 (td, J=10.8, 4.8 Hz, 1H), 4.11 (dd, J=11.8, 4.8 Hz, 1H), 3.90 (d, J=2.8 Hz, 1H), 3.72-3.63 (m, 2H), 3.59-3.56 (m, 1H), 3.42-3.28 (m, 7H), 3.26-3.05 (m, 3H), 3.02-2.69 (m, 6H).

### Preparation of (R)-1-((2R,3R,4R,5R)-3-hydroxy-5-(2-methoxyethoxy)-4-((6-(trifluoromethyl)pyrazin-2-yl)amino)tetrahydrofuran-2-yl)ethane-1,2-diol (A126)

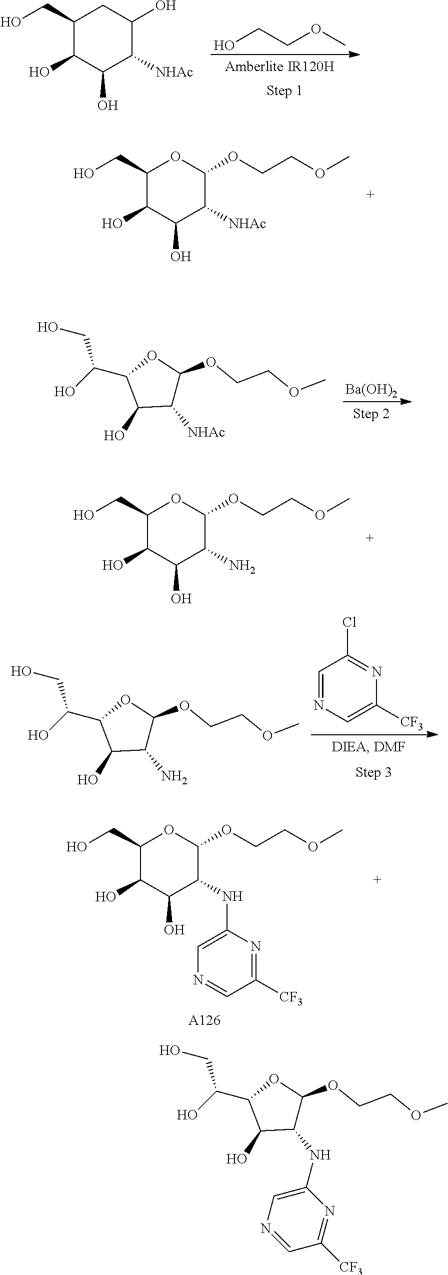

Step 1: To a solution of N-((3R,4R,5R,6R)-2,4,5-trihydroxy-6-(hydroxymethyl)tetrahydro-2H-pyran-3-yl)acetamide (3.3 g, 14 mmol) in 2-methoxyethan-1-ol (30 mL) was added Amberlite IR 120 H⁺ (1.8 g) at an ambient temperature. The mixture was stirred at 70° C. for 2 days. The mixture was filtered, and the filtrate was concentrated in vacuo. The crude product was purified by flash silica column chromatography (eluent=0-12% MeOH in DCM) to give a mixture (1.5 g, 36% yield) as white solid. The mixture (280 mg) was further purified by SFC (Column: ChiralPak IG, 250×21.2 mm I.D., 5 μm; Mobile phase: A for CO₂ and B for MeOH+0.1% NH₃·H₂O; Gradient: B 20%) giving N-((2S,3R,4R,5R,6R)-4,5-dihydroxy-6-(hydroxymethyl)-2-(2-methoxyethoxy)tetrahydro-2H-pyran-3-yl)acetamide (90 mg) as colorless oil and N-((2R,3R,4R,5R)-5-((R)-1,2-dihydroxyethyl)-4-hydroxy-2-(2-methoxyethoxy)tetrahydrofuran-3-yl)acetamide (47 mg) as white solid. LC-MS (ESI) found: 280 [M+H]⁺. ¹H NMR (400 MHz, CD₃OD) for N-((2S,3R,4R,5R,6R)-4,5-dihydroxy-6-(hydroxymethyl)-2-(2-methoxyethoxy)tetrahydro-2H-pyran-3-yl)acetamide: δ 4.82 (d, J=3.6 Hz, 1H), 4.28 (dd, J=10.9, 3.6 Hz, 1H), 3.88 (d, J=2.5 Hz, 1H), 3.85-3.79 (m, 2H), 3.76 (dd, J=10.9, 3.2 Hz, 1H) 3.73-3.66 (m, 2H), 3.63-3.55 (m, 3H), 3.39 (s, 3H), 1.99 (s, 3H). ¹H NMR (400 MHz, CD₃OD) for N-((2R,3R,4R,5R)-5-((R)-1,2-dihydroxyethyl)-4-hydroxy-2-(2-methoxyethoxy)tetrahydrofuran-3-yl)acetamide: δ 4.90 (d, J=1.5 Hz, 1H), 4.18-4.14 (m, 1H) 4.06-4.01 (m, 1H), 3.98 (dd, J=6.4, 2.7 Hz, 1H), 3.81 (dd, J=9.7, 4.3 Hz, 1H), 3.75-3.70 (m, 1H), 3.63 (s, 1H), 3.62-3.60 (m, 1H), 3.60-3.52 (m, 3H), 3.37 (s, 3H), 1.96 (s, 3H).
Step 2: To a solution of mixture obtained from Step 1 (940 mg, 3.366 mmol) in H₂O (10 mL) was added Ba(OH)₂ (5.7 g, 33.657 mmol). The reaction was stirred at 120° C. overnight. To the mixture was added (NH₄)₂SO₄ (4.4 g, 33.657 mmol). The mixture was stirred at 120° C. for 2 h. The reaction was cooled to rt. The mixture was filtered and the filtrate was concentrated in vacuo. The pH of the crude was adjusted to ˜7 by adding NaOMe through the environment of MeOH. The mixture was filtered, and the filtrate was concentrated in vacuo to give crude mixture (660 mg, 83% yield) as yellow oil. LC-MS (ESI) found: 238 [M+H]⁺.
Step 3: To a solution of the mixture obtained from Step 2 (660 mg, 2.782 mmol) in DMF (8 mL) were added 2-chloro-6-(trifluoromethyl)pyrazine (609 mg, 3.338 mmol) and DIEA (0.460 mL, 2.782 mmol). The reaction was stirred at 90° C. overnight. The mixture was concentrated and the residue was purified by C18 column (eluent=80% MeOH in H₂O) to give (2R,3R,4R,5R,6S)-2-(hydroxymethyl)-6-(2-methoxyethoxy)-5-((6-(trifluoromethyl)pyrazin-2-yl)amino)tetrahydro-2H-pyran-3,4-diol (10 mg, 0.94% yield) as yellow oil and (R)-1-((2R,3R,4R,5R)-3-hydroxy-5-(2-methoxyethoxy)-4-((6-(trifluoromethyl)pyrazin-2-yl)amino)tetrahydrofuran-2-yl)ethane-1,2-diol (14 mg, 1.31% yield) as white solid. LC-MS (ESI) found: 384 [M+H]⁺. ¹H NMR (400 MHz, CD₃OD) for (2R,3R,4R,5R,6S)-2-(hydroxymethyl)-6-(2-methoxyethoxy)-5-((6-(trifluoromethyl)pyrazin-2-yl)amino)tetrahydro-2H-pyran-3,4-diol: δ 5.00 (d, J=3.5 Hz, 1H) 4.51 (dd, J=10.6, 3.2 Hz, 1H), 3.97-3.88 (m, 1H), 3.83 (dd, J=5.7, 3.9 Hz, 1H), 3.81-3.77 (m, 1H), 3.74 (dd, J=12.8, 6.6 Hz, 1H), 3.57 (dd, J=12.4, 5.4 Hz, 1H), 3.31 (s, 1H). ¹H NMR (400 MHz, CD₃OD) for (R)-1-((2R,3R,4R,5R)-3-hydroxy-5-(2-methoxyethoxy)-4-((6-(trifluoromethyl)pyrazin-2-yl)amino)tetrahydrofuran-2-yl)ethane-1,2-diol: δ 8.15 (s, 1H), 8.07 (s, 1H), 4.99 (d, J=1.3 Hz, 1H), 4.31 (d, J=2.0 Hz, 1H), 4.18-4.13 (m, 1H), 4.07 (dd, J=6.4, 2.7 Hz, 1H), 3.88-3.82 (m, 1H), 3.81-3.73 (m, 1H), 3.62 (ddd, J=13.2, 8.0, 4.2 Hz, 5H), 3.36 (s, 3H).

### Preparation of 2-(3-(2-((((2R,3R,4R,5S)-3,4-dihydroxy-5-((6-(trifluoromethyl)pyrazin-2-yl)amino)tetrahydro-2H-pyran-2-yl)methyl)amino)-2-oxoethyl)phenoxy)acetic acid (A127)

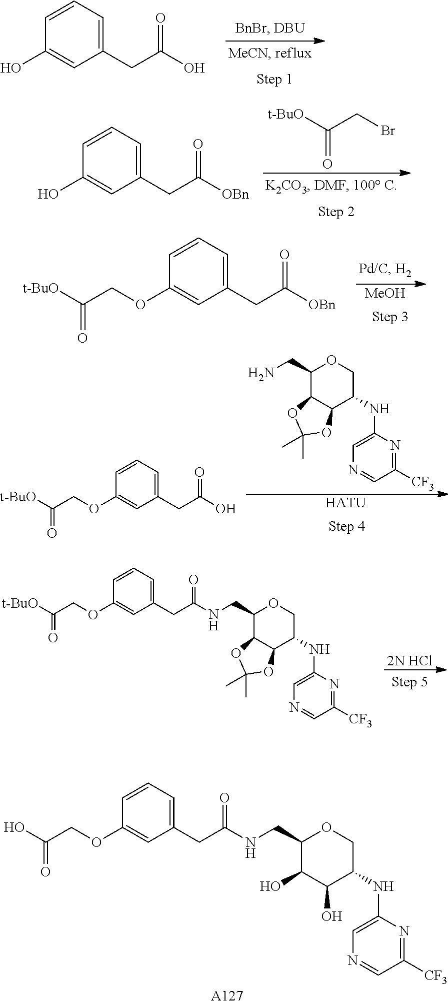

Step 1: To a solution of 2-(3-hydroxyphenyl)acetic acid (2.1 g, 13.8 mmol) in dry CH₃CN (30 mL) was added DBU (2.56 g, 16.8 mmol) and benzyl bromide (2.61 g, 15.4 mmol) at an ambient temperature. The mixture was stirred at 90° C. for 18 h. Then the reaction mixture was concentrated and purified by silica gel (PE/EA=5/1) to give benzyl 2-(3-hydroxyphenyl)acetate (2.1 g, 62% yield). LC-MS (ESI) found: 243 [M+H]⁺.
Step 2: To a solution of benzyl 2-(3-hydroxyphenyl)acetate (2.1 g, 8.6 mmol) in CH₃CN (20 ml) was added tert-butyl 2-bromoacetate (2.52 g, 13 mmol) and K₂CO₃ (2.39 g, 17.3 mmol). The mixture was stirred at 80° C. for 18 h. The reaction mixture was filtered and the solvent was concentrated. The residue was purified by silica gel (petroleum ether/ethyl acetate=5/1) to give benzyl 2-(3-(2-(tert-butoxy)-2-oxoethoxy)phenyl)acetate (2.9 g, 86% yield). LC-MS (ESI) found: 374 [M+18]⁺.
Step 3: To a solution of benzyl 2-(3-(2-(tert-butoxy)-2-oxoethoxy)phenyl)acetate (2.9 g, 8.1 mmol) in MeOH (30 mL) were added Pd/C (300 mg, 10% wt., 60% wet). The mixture was stirred at an ambient temperature under a H2 balloon for 0.5 h. The mixture was filtered through a Celite pad, and the organic layer was concentrated to give crude 2-(3-(2-(tert-butoxy)-2-oxoethoxy)phenyl)acetic acid (1.9 g, crude). LC-MS (ESI) found: 267 [M+H]⁺.
Step 4: A solution of 2-(3-(2-(tert-butoxy)-2-oxoethoxy)phenyl)acetic acid (0.93 g, 3.52 mmol) and HATU (1.83 g, 4.8 mmol) in DMF (5 mL) was stirred at an ambient temperature for 2 h. N-((3aS,4R,7S,7aR)-4-(aminomethyl)-2,2-dimethyltetrahydro-4H-[1,3]dioxolo[4,5-c]pyran-7-yl) 6-(trifluoromethyl)pyrazin-2-amine (1.11 g, 3.2 mmol) and DIEA (1.058 mL, 6.4 mmol) was added to above mixture. Reaction mixture was stirred at an ambient temperature overnight. Reaction was concentrated. Residue was purified by flash chromatography (MeOH:DCM=1:15) to give tert-butyl 2-(3-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)phenoxy)acetate as a yellow oil (1.7 g, 89% yield). LC-MS (ESI) found: 597[M+H]⁺.
Step 5: A solution of tert-butyl 2-(3-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)-2-oxoethyl)phenoxy)acetate (1.7 g, 2.86 mmol) in HCl (5 mL, 2 N in H₂O) was stirred rt overnight. Reaction mixture was concentrated. Residue was purified by flash chromatography revered phase (MeOH in water from 5% to 60%) to give 2-(3-(2-((((2R,3R,4R,5S)-3,4-dihydroxy-5-((6-(trifluoromethyl)pyrazin-2-yl)amino)tetrahydro-2H-pyran-2-yl)methyl)amino)-2-oxoethyl)phenoxy)acetic acid as a white solid (1.29 g, 90% yield). LC-MS (ESI) found: 501[M+H]⁺. ¹H NMR (400 MHz, CD₃OD): δ 8.10 (s, 1H), 7.99 (s, 1H), 7.22 (t, J=7.8 Hz, 1H), 6.96-6.88 (m, 2H), 6.85-6.78 (m, 1H), 4.64 (s, 2H), 4.31 (td, J=10.5, 5.1 Hz, 1H), 4.11 (dd, J=11.0, 5.1 Hz, 1H), 3.80 (d, J=3.0 Hz, 1H), 3.62 (dd, J=10.3, 3.2 Hz, 1H) 3.51-3.45 (m, 3H), 3.34-3.30 (m, 2H), 3.08 (t, J=10.8 Hz, 1H).

### Preparation of 7-((((2R,3R,4R,5S)-3,4-dihydroxy-5-((6-(trifluoromethyl)pyrazin-2-yl)amino)tetrahydro-2H-pyran-2-yl)methyl)carbamoyl)-9-oxo-9H-fluorene-2-carboxylic acid (A128)

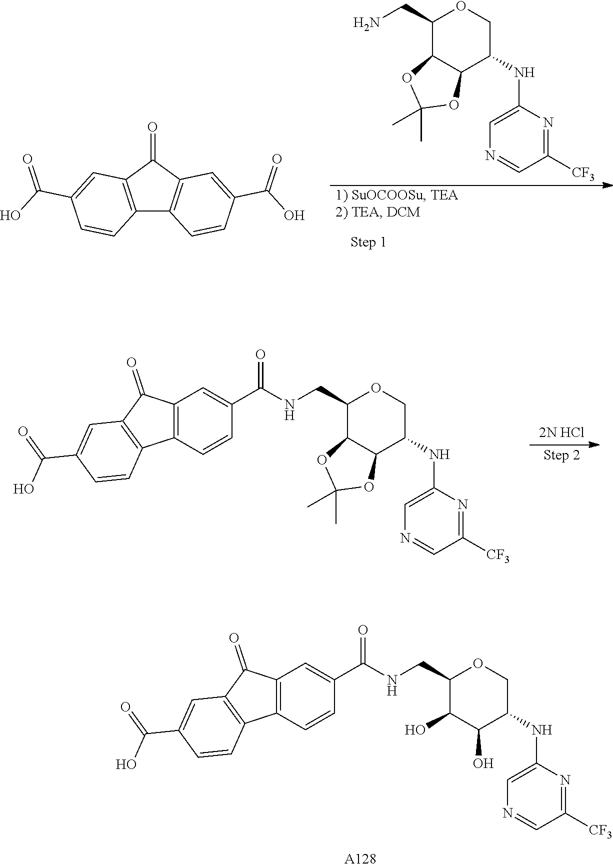

Step 1: To a solution of 9-oxo-9H-fluorene-2,7-dicarboxylic acid (536 mg, 2.0 mmol) in DCM (40 mL) was added Et₃N (605.5 mg, 6.0 mmol) and bis(2,5-dioxopyrrolidin-1-yl) carbonate (1.1 g, 4.0 mmol) slowly at an ambient temperature. The reaction was stirred at an ambient temperature for 5 h. The mixture was used for next step without purification. Then a solution of N-[(3aS,4R,7S,7aR)-4-(aminomethyl)-2,2-dimethyl-hexahydro-[1,3]dioxolo[4,5-c]pyran-7-yl]-6-(trifluoromethyl)pyrazin-2-amine (250 mg, 0.7 mmol) in DCM (20 mL) was added and the reaction was stirred at an ambient temperature for 18 h. Then the reaction was concentrated and purified by reverse phase with MeOH/H₂O to give 7-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-9-oxo-9H-fluorene-2-carboxylic acid (180 mg, 43% yield) as yellow solid. LC-MS (ESI) found: 598.7 [M+H]⁺.
Step 2: To a solution of 7-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)carbamoyl)-9-oxo-9H-fluorene-2-carboxylic acid (180 mg, 0.3 mmol) in THF (5 mL) was added HCl solution (2 mL, 2N in H₂O) at an ambient temperature. The reaction was stirred at an ambient temperature for 18 h. Then the reaction was concentrated and purified by reverse phase with H₂O (0.1% NH₃·H₂O)/CH₃CN to give 7-((((2R,3R,4R,5S)-3,4-dihydroxy-5-((6-(trifluoromethyl)pyrazin-2-yl)amino)tetrahydro-2H-pyran-2-yl)methyl)carbamoyl)-9-oxo-9H-fluorene-2-carboxylic acid (74.1 mg, 44% yield) as yellow solid. LC-MS (ESI) found: 558.7 [M+H]⁺. 1H NMR (400 MHz, CD₃OD): δ 8.13-8.26 (m, 2H), 8.04-8.14 (m, 3H), 7.96-8.02 (m, 2H), 7.82 (dd, J=16.0, 8.4 Hz, 2H), 4.33-4.39 (m, 1H) 4.15 (dd, J=11.2, 5.2 Hz, 1H), 3.93 (d, J=4.2 Hz, 1H), 3.67-3.74 (m, 3H), 3.55 (dd, J=12.8, 7.2 Hz, 1H), 3.14 (t, J=10.8 Hz, 1H).

### Preparation of 3,3′-((2-aminopropane-1,3-diyl)bis(oxy))bis(N-(2-(2-(2-(2-(((2R,3R,4R,5R,6R)-4,5-dihydroxy-6-(hydroxymethyl)-3-((6-(trifluoromethyl)pyrazin-2-yl)amino)tetrahydro-2H-pyran-2-yl)oxy)ethoxy)ethoxy)ethoxy)ethyl)propanamide) (A129)

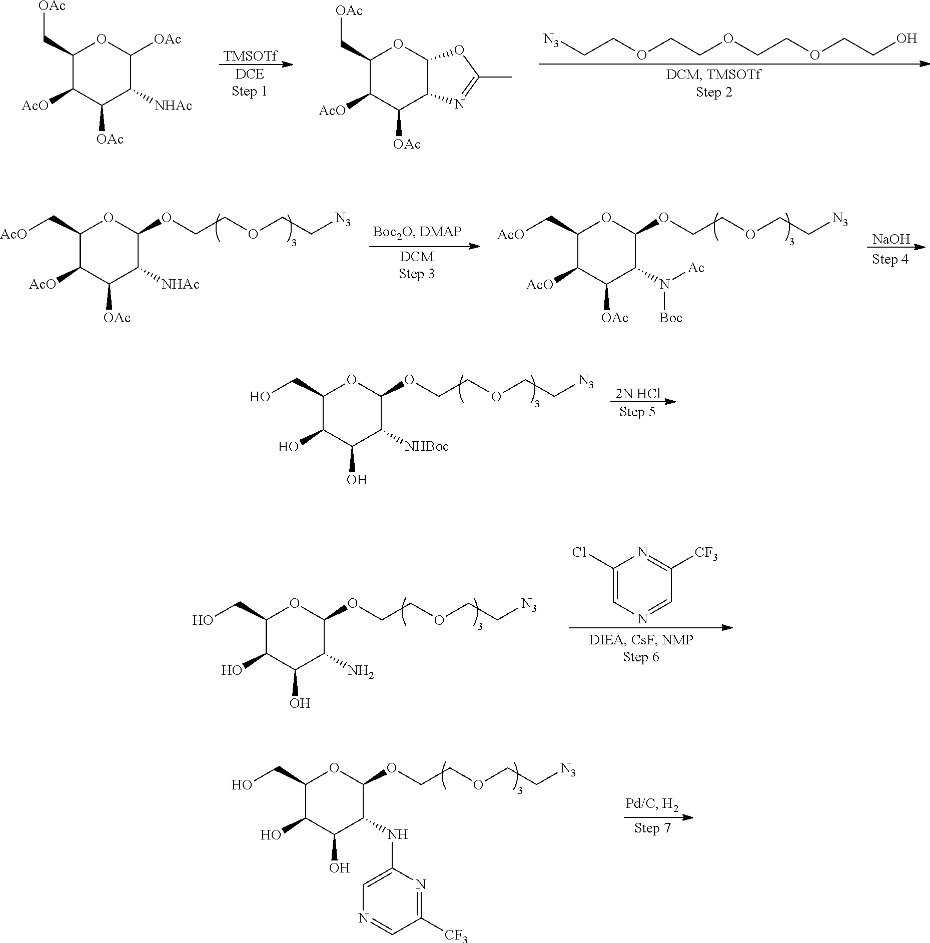

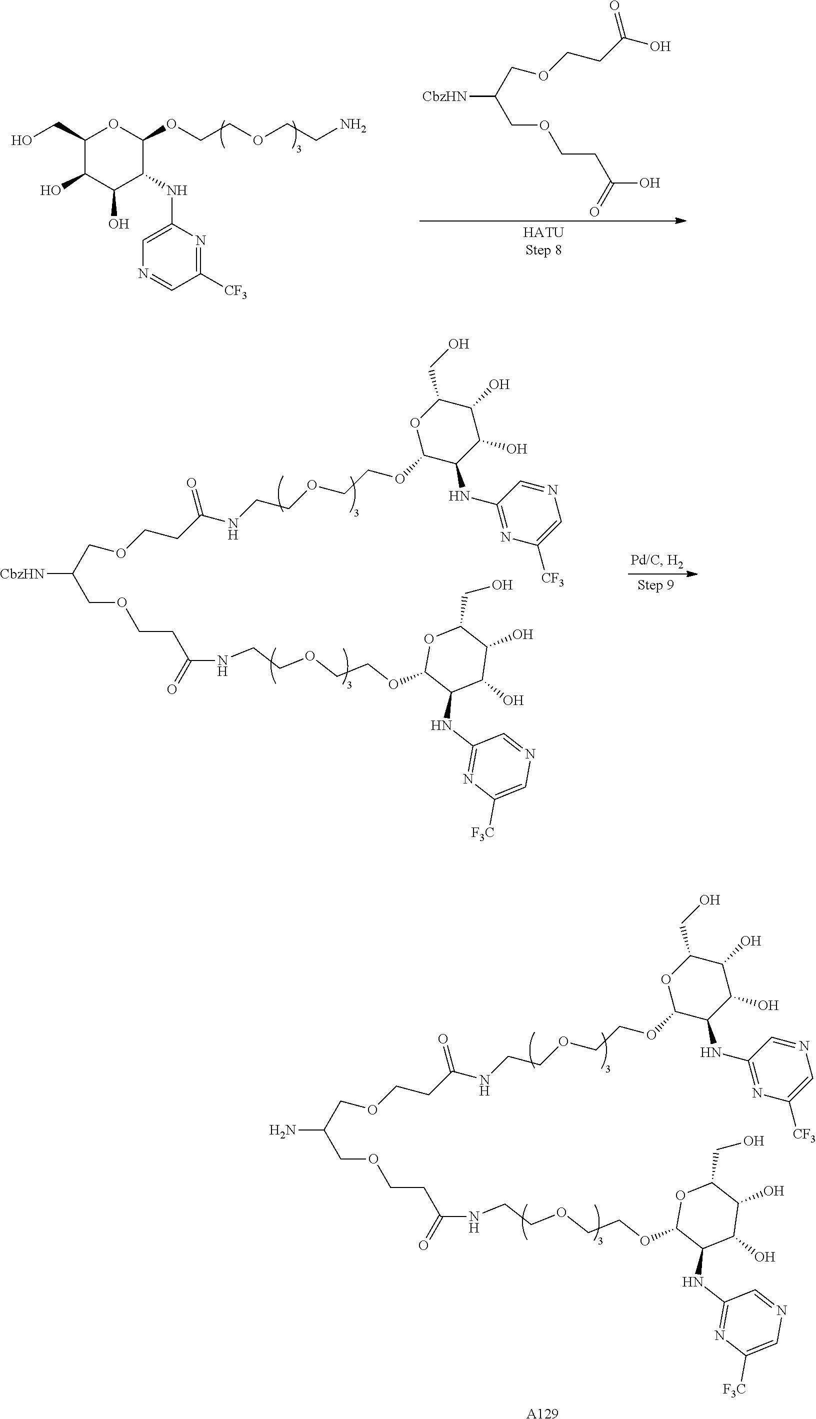

Step 1: (3R,4R,5R,6R)-3-acetamido-6-(acetoxymethyl)tetrahydro-2H-pyran-2,4,5-triyl triacetate (5 g, 15.2 mmol) was dissolved in DCE (50 mL). Then molecular sieve (0.5 g) and TMSOTf (6.74 g, 30.4 mmol) were added at 0° C. The reaction mixture was stirred at an ambient temperature for 2 hours. The pH of the reaction mixture was adjusted to 7.5 with TEA at 0° C. The mixture was then washed with H₂O. The organic phase was separated and concentrated to give crude (3aR,5R,6R,7R,7aR)-5-(acetoxymethyl)-2-methyl-3a,6,7,7a-tetrahydro-5H-pyrano[3,2-d]oxazole-6,7-diyl diacetate (3.5 g). LC-MS (ESI) found: 330 [M+H]⁺.
Step 2: To a solution of (3aR,5R,6R,7R,7aR)-5-(acetoxymethyl)-2-methyl-3a,6,7,7a-tetrahydro-5H-pyrano[3,2-d]oxazole-6,7-diyl diacetate (1.5 g, 4.6 mmol) in DCM (15 mL) were added molecular sieve (0.8 g) and 2-(2-(2-(2-azidoethoxy)ethoxy)ethoxy)ethan-1-ol and (1 g, 4.6 mmol). Then TMSOTf (0.5 g, 2.3 mmol) was added at 0° C. The reaction mixture was stirred at an ambient temperature for 16 hours. The reaction mixture was adjusted to pH=7.5 with TEA at 0° C. The reaction was concentrated, purified by flash (petroleum ether: ethyl acetate=2:1) to give (2R,3R,4R,5R,6R)-5-acetamido-2-(acetoxymethyl)-6-(2-(2-(2-(2-azidoethoxy)ethoxy)ethoxy)ethoxy)tetrahydro-2H-pyran-3,4-diyl diacetate (1.2 g, 48% yield). LC-MS (ESI) found: 549 [M+H]⁺.
Step 3: To a suspension of (2R,3R,4R,5R,6R)-5-acetamido-2-(acetoxymethyl)-6-(2-(2-(2-(2-azidoethoxy)ethoxy)ethoxy)ethoxy)tetrahydro-2H-pyran-3,4-diyl diacetate (200 mg, 0.365 mmol) in dry DCM (5 mL) were added Boc₂O (398 mg, 1.823 mmol) and TEA (0.101 mL, 0.729 mmol) at 0° C. The mixture was stirred at an ambient temperature for 16 h. ethyl acetate/H₂O was added, and the organic phase was separated and purified by flash (petroleum ether: ethyl acetate=5:1) to give (2R,3R,4R,5R,6R)-2-(acetoxymethyl)-6-(2-(2-(2-(2-azidoethoxy)ethoxy)ethoxy)ethoxy)-5-(N-(tert-butoxycarbonyl)acetamido)tetrahydro-2H-pyran-3,4-diyl diacetate (170 mg, 34% yield). LC-MS (ESI) found: 649 [M+H]⁺.
Step 4: To a suspension of (2R,3R,4R,5R,6R)-2-(acetoxymethyl)-6-(2-(2-(2-(2-azidoethoxy)ethoxy)ethoxy)ethoxy)-5-(N-(tert-butoxycarbonyl)acetamido)tetrahydro-2H-pyran-3,4-diyl diacetate (180 mg, 0.277 mmol) in THF (3 mL) and H₂O (3 mL) was added NaOH (23 mg, 0.555 mmol). The mixture was stirred at 60° C. for 16 h. The solvent was removed and the residue was purified by flash (DCM:MeOH=10:1) to give tert-butyl ((2R,3R,4R,5R,6R)-2-(2-(2-(2-(2-azidoethoxy)ethoxy)ethoxy)ethoxy)-4,5-dihydroxy-6-(hydroxymethyl)tetrahydro-2H-pyran-3-yl)carbamate (80 mg, 60% yield). LC-MS (ESI) found: 481 [M+H]⁺.
Step 5: To a suspension of tert-butyl ((2R,3R,4R,5R,6R)-2-(2-(2-(2-(2-azidoethoxy)ethoxy)ethoxy)ethoxy)-4,5-dihydroxy-6-(hydroxymethyl)tetrahydro-2H-pyran-3-yl)carbamate (97 mg, 0.202 mmol) in THF (1 mL) was added HCl solution (1 mL, 2 N in H₂O) was added. The mixture was stirred at an ambient temperature for 16 h and the solvent was removed to give crude (2R,3R,4R,5R,6R)-5-amino-6-(2-(2-(2-(2-azidoethoxy)ethoxy)ethoxy)ethoxy)-2-(hydroxymethyl)tetrahydro-2H-pyran-3,4-diol (76 mg, 99% yield). LC-MS (ESI) found: 381 [M+H]⁺.
Step 6: To a solution of (2R,3R,4R,5R,6R)-5-amino-6-(2-(2-(2-(2-azidoethoxy)ethoxy)ethoxy)ethoxy)-2-(hydroxymethyl)tetrahydro-2H-pyran-3,4-diol (588 mg, 1.546 mmol) in NMP (5 mL) were added 2-chloro-6-(trifluoromethyl)pyrazine (564 mg, 3.091 mmol), CsF (117 mg, 0.773 mmol) and DIEA (1.533 mL, 9.274 mmol). The reaction was stirred at 90° C. for 18 h. The mixture was directly purified by C18 column to give (2R,3R,4R,5R,6R)-6-(2-(2-(2-(2-azidoethoxy)ethoxy)ethoxy)ethoxy)-2-(hydroxymethyl)-5-((6-(trifluoromethyl)pyrazin-2-yl)amino)tetrahydro-2H-pyran-3,4-diol (280 mg, 34% yield). LC-MS (ESI) found: 527 [M+H]⁺.
Step 7: To a solution of (2R,3R,4R,5R,6R)-6-(2-(2-(2-(2-azidoethoxy)ethoxy)ethoxy)ethoxy)-2-(hydroxymethyl)-5-((6-(trifluoromethyl)pyrazin-2-yl)amino)tetrahydro-2H-pyran-3,4-diol (100 mg, 0.190 mmol) in MeOH (3 mL) was added Pd/C (20 mg, 10% wt., 60% wet). The mixture was stirred at an ambient temperature for 0.5 h under a H2 balloon. The mixture was filtered through a Celite pad, and the residue was concentrated to give crude (2R,3R,4R,5R,6R)-6-(2-(2-(2-(2-aminoethoxy)ethoxy)ethoxy)ethoxy)-2-(hydroxymethyl)-5-((6-(trifluoromethyl)pyrazin-2-yl)amino)tetrahydro-2H-pyran-3,4-diol (95 mg, 99% yield). LC-MS (ESI) found: 501 [M+H]⁺.
Step 8: To a solution of 3,3′-((2-(((benzyloxy)carbonyl)amino)propane-1,3-diyl)bis(oxy))dipropionic acid (32 mg, 0.087 mmol) in DMF (2 mL) were added HATU (72 mg, 0.191 mmol) and DIEA (0.10 mL, 0.606 mmol). The mixture was stirred at 0° C. for 0.5 h. Then (2R,3R,4R,5R,6R)-6-(2-(2-(2-(2-aminoethoxy)ethoxy)ethoxy)ethoxy)-2-(hydroxymethyl)-5-((6-(trifluoromethyl)pyrazin-2-yl)amino)tetrahydro-2H-pyran-3,4-diol (95 mg, 0.191 mmol) in DMF (0.5 mL) was added. The mixture was stirred at an ambient temperature for 16 h. The reaction mixture was purified by C18 column to give benzyl (1,35-bis(((2R,3R,4R,5R,6R)-4,5-dihydroxy-6-(hydroxymethyl)-3-((6-(trifluoromethyl)pyrazin-2-yl)amino)tetrahydro-2H-pyran-2-yl)oxy) 13,23-dioxo-3,6,9,16,20,27,30,33-octaoxa-12,24-diazapentatriacontan-18-yl)carbamate (72 mg, 62% yield). LC-MS (ESI) found: 1334 [M+H]⁺.
Step 9: To a solution of benzyl (1,35-bis(((2R,3R,4R,5R,6R)-4,5-dihydroxy-6-(hydroxymethyl) 3-((6-(trifluoromethyl)pyrazin-2-yl)amino)tetrahydro-2H-pyran-2-yl)oxy)-13,23-dioxo-3,6,9,16,20,27,30,33-octaoxa-12,24-diazapentatriacontan-18-yl)carbamate (72 mg, 0.054 mmol) in MeOH (3 mL) was added Pd/C (10 mg, 10% wt., 60% wet). The mixture was stirred at an ambient temperature for 0.5 h under a H2 balloon. The mixture was filtered through a Celite pad. The filtrate was concentrated and the residue was purified by C18 column to give 3,3′-((2-aminopropane-1,3-diyl)bis(oxy))bis(N-(2-(2-(2-(2-(((2R,3R,4R,5R,6R)-4,5-dihydroxy-6-(hydroxymethyl)-3-((6-(trifluoromethyl)pyrazin-2-yl)amino)tetrahydro-2H-pyran-2-yl)oxy)ethoxy)ethoxy)ethoxy)ethyl)propanamide) (50 mg, 77% yield). LC-MS (ESI) found: 1200 [M+H]⁺. ¹H NMR (400 MHz, CD₃OD): δ 8.19 (s, 2H), 8.00 (s, 2H), 4.56 (d, J=8.3 Hz, 2H), 4.22-4.09 (m, 2H), 3.99-3.86 (m, 4H), 3.83-3.65 (m, 12H), 3.63-3.34 (m, 34H), 3.17 (s, 1H) 2.44 (t, J=5.9 Hz, 4H).

### Preparation of tert-butyl 4-(2-((((2R,3R,4R,5S)-3,4-dihydroxy-5-((6-(trifluoromethyl)pyrazin-2-yl)amino)tetrahydro-2H-pyran-2-yl)methyl)amino)ethyl)piperidine-1-carboxylate (A130)

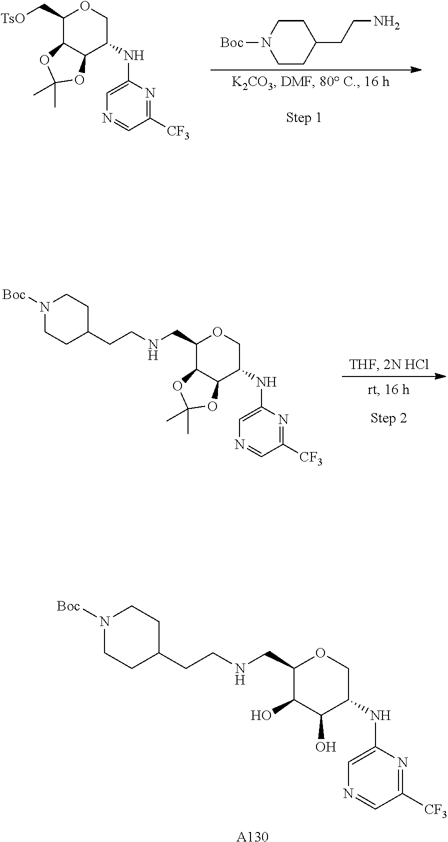 [SMILES: Cc1ccc(S(=O)(=O)OC[C@H]2OC[C@H](Nc3cncc(C(F)(F)F)n3)[C@H]3OC(C)(C)O[C@H]32)cc1 ; CC(C)(C)OC(=O)N1CCC(CCN)CC1 ; CC(C)(C)OC(=O)N1CCC(CCNC[C@H]2OC[C@H](Nc3cncc(C(F)(F)F)n3)[C@H]3OC(C)(C)O[C@H]32)CC1 ; CC(C)(C)OC(=O)N1CCC(CCNC[C@H]2OC[C@H](Nc3cncc(C(F)(F)F)n3)[C@@H](O)[C@H]2O)CC1 ; "A130"]

Step 1: To a mixture of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl 4-methylbenzenesulfonate (100 mg, 0.2 mmol) and tert-butyl 4-(2-aminoethyl)piperidine-1-carboxylate (91 mg, 0.4 mmol) in DMF (5 mL) was added potassium carbonate (137 mg, 1.0 mmol) at an ambient temperature. The reaction mixture was stirred at 80° C. overnight. After completion, to the mixture was added H₂O (50 mL) and it was extracted with ethyl acetate (3×10 mL). The combined organic layers were washed with brine (10 mL), dried over anhydrous Na₂SO₄, and filtered. The filtrate was concentrated and the residue was purified by prep-TLC (DCM:MeOH=10:1) to give tert-butyl 4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)ethyl)piperidine-1-carboxylate (70 mg, 57% yield) as a colorless oil. LC-MS (ESI) found: 560 [M+H]⁺.
Step 2: To a mixture of tert-butyl 4-(2-((((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)amino)ethyl)piperidine-1-carboxylate (70 mg, 0.12 mmol) in THF (5 mL) was added drops of HCl solution (2N in H₂O) at an ambient temperature. The mixture was stirred at an ambient temperature overnight. After completion, NH₃·H₂O (0.1 mL) was added. The mixture was concentrated and the residue was purified by C18 column to give tert-butyl 4-(2-((((2R,3R,4R,5S)-3,4-dihydroxy-5-((6-(trifluoromethyl)pyrazin-2-yl)amino)tetrahydro-2H-pyran-2-yl)methyl)amino)ethyl)piperidine-1-carboxylate (23 mg, 35% yield) as a white solid. LC-MS (ESI) found: 520 [M+H]⁺. ¹H NMR (400 MHz, CD₃OD): δ 8.11 (s, 1H), 8.00 (s, 1H), 4.33 (td, J=10.5, 5.0 Hz, 1H), 4.14 (dd, J=11.0, 5.1 Hz, 1H), 4.04 (d, J=13.2 Hz, 2H), 3.86 (d, J=2.6 Hz, 1H), 3.65 (dd, J=10.5, 3.0 Hz, 1H), 3.55 (dd, J=7.8, 3.1 Hz, 1H), 3.12 (t, J=10.8 Hz, 1H), 2.98 (dd, J=12.6, 8.1 Hz, 1H), 2.83-2.64 (m, 5H), 1.70 (d, J=12.5 Hz, 2H), 1.49 (d, J=6.6 Hz, 2H) 1.44 (s, 9H), 1.31 (d, J=18.0 Hz, 1H), 1.17-1.00 (m, 2H).

### Preparation of 3,3′-((2-amino-2-(1-((2R,3R,4R,5S)-3,4-dihydroxy-5-((6-(trifluoromethyl)pyrazin-2-yl)amino)tetrahydro-2H-pyran-2-yl)-15-oxo-2,5,8,11,18-pentaoxa-14-azanonadecan-19-yl)propane-1,3-diyl)bis(oxy))bis(N-(1-((2R,3R,4R,5S)-3,4-dihydroxy-5-((6-(trifluoromethyl)pyrazin-2-yl)amino)tetrahydro-2H-pyran-2-yl)-2,5,8,11-tetraoxatridecan-13-yl)propanamide) (A131)

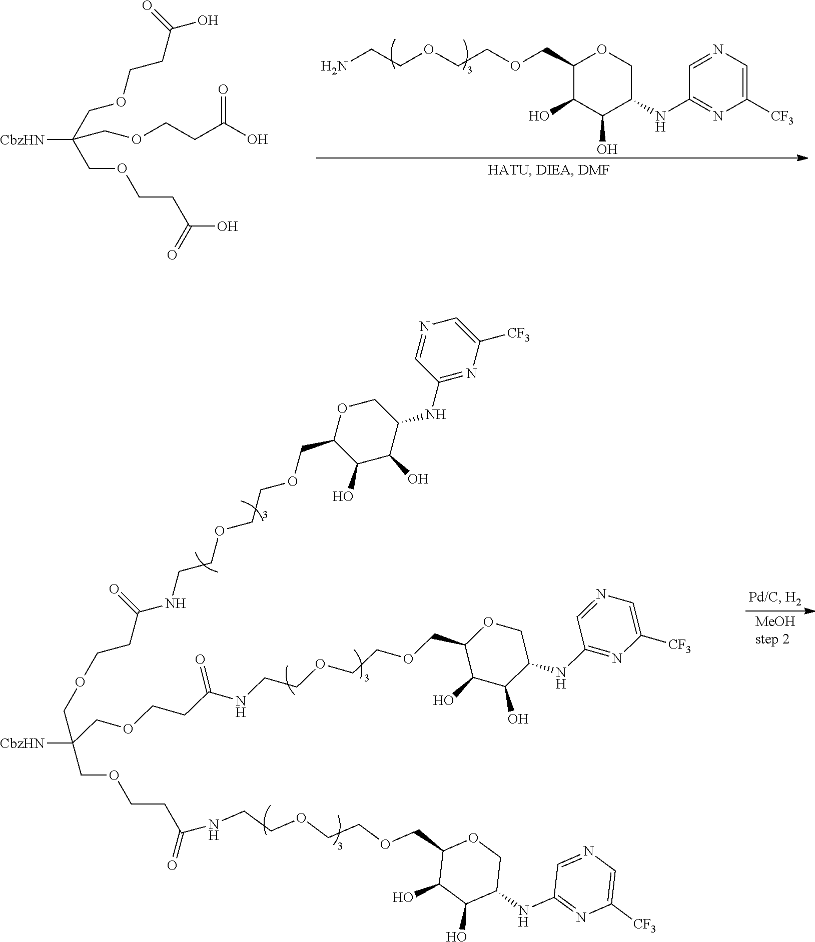

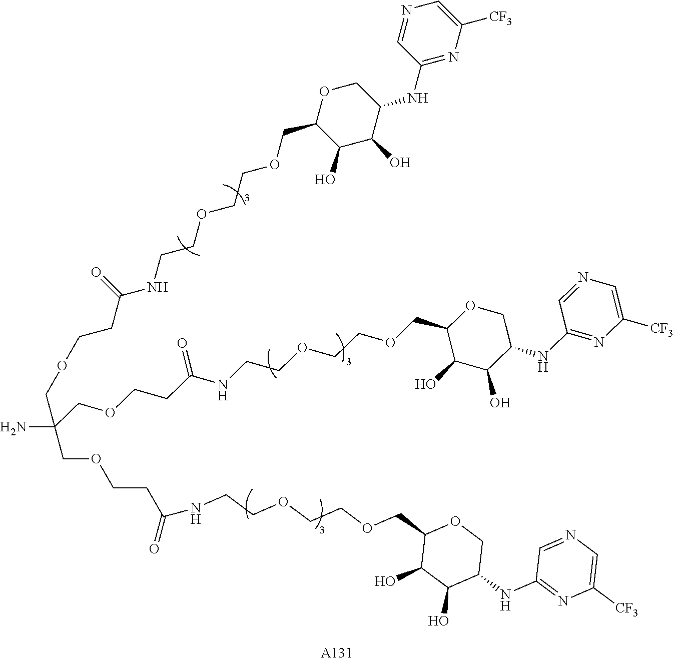

Step 1: A solution of 3,3′-((2-(((benzyloxy)carbonyl)amino)-2-((2-carboxyethoxy)methyl)propane-1,3-diyl)bis(oxy))dipropionic acid (310 mg, 0.66 mmol), HATU (875.1 mg, 2.3 mmol) and DIEA (0.54 mL, 3.29 mmol) in DMF (15 mL) was stirred at an ambient temperature for 30 min. (2R,3R,4R,5S)-2-(13-amino-2,5,8,11-tetraoxatridecyl)-5-((6-(trifluoromethyl)pyrazin-2-yl)amino)tetrahydro-2H-pyran-3,4-diol (1274.2 mg, 2.63 mmol) was added at an ambient temperature. The reaction was stirred overnight. The resulting mixture was concentrated in vacuo. The crude product was purified by Prep-HPLC to give benzyl (1,39-bis((2R,3R,4R,5S)-3,4-dihydroxy-5-((6-(trifluoromethyl)pyrazin-2-yl)amino)tetrahydro-2H-pyran-2-yl)-20-(1-((2R,3R,4R,5S)-3,4-dihydroxy-5-((6-(trifluoromethyl)pyrazin-2-yl)amino)tetrahydro-2H-pyran-2-yl)-15-oxo-2,5,8,11,18-pentaoxa-14-azanonadecan-19-yl) 15,25-dioxo-2,5,8,11,18,22,29,32,35,38-decaoxa-14,26-diazanonatriacontan-20-yl)carbamate (950 mg, yield: 77.2%) as a white solid. LC-MS (ESI) found: 1871 [M+H]⁺.
Step 2: A solution of benzyl (1,39-bis((2R,3R,4R,5S)-3,4-dihydroxy-5-((6-(trifluoromethyl)pyrazin-2-yl)amino)tetrahydro-2H-pyran-2-yl)-20-(1-((2R,3R,4R,5S)-3,4-dihydroxy-5-((6-(trifluoromethyl)pyrazin-2-yl)amino)tetrahydro-2H-pyran-2-yl)-15-oxo-2,5,8,11,18-pentaoxa-14-azanonadecan-19-yl)-15,25-dioxo-2,5,8,11,18,22,29,32,35,38-decaoxa-14,26-diazanonatriacontan-20-yl)carbamate (950 mg, 0.51 mmol) in MeOH (10 mL) was added Pd/C (100 mg, 10% purity) at an ambient temperature under H2 (15 Psi). The reaction was stirred at an ambient temperature for 18 h. The resulting mixture was filtered and concentrated in vacuo. The crude product was purified by prep-HPLC to give 3,3′-((2-amino-2-(1-((2R,3R,4R,5S)-3,4-dihydroxy-5-((6-(trifluoromethyl)pyrazin-2-yl)amino)tetrahydro-2H-pyran-2-yl)-15-oxo-2,5,8,11,18-pentaoxa-14-azanonadecan-19-yl)propane-1,3-diyl)bis(oxy))bis(N-(1-((2R,3R,4R,5S)-3,4-dihydroxy-5-((6-(trifluoromethyl) pyrazin-2-yl)amino)tetrahydro-2H-pyran-2-yl)-2,5,8,11-tetraoxatridecan-13-yl)propanamide) (198.3 mg, yield: 22.5%) as a white solid. LC-MS (ESI) found: 1737 [M+H]⁺. ¹H NMR (400 MHz, CD₃OD): δ 8.12 (s, 3H), 8.00 (s, 3H), 4.33 (td, J=10.6, 5.2 Hz, 3H), 4.12 (dd, J=11.0, 5.2 Hz, 3H), 3.93 (d, J=3.0 Hz, 3H), 3.70-3.60 (m, 54H), 3.55 (t, J=5.5 Hz, 6H), 3.40-3.34 (m, 12H), 3.12 (t, J=10.8 Hz, 3H), 2.45 (t, J=6.0 Hz, 6H). ¹⁹F NMR (377 MHz, CD₃OD): δ −70.21 (s).

### Preparation of tert-butyl (6-(2-((2R,3R,4R,5S)-3,4-dihydroxy-5-((6-(trifluoromethyl)pyrazin-2-yl)amino)tetrahydro-2H-pyran-2-yl)acetamido)spiro[3.3]heptan-2-yl)carbamate (A132)

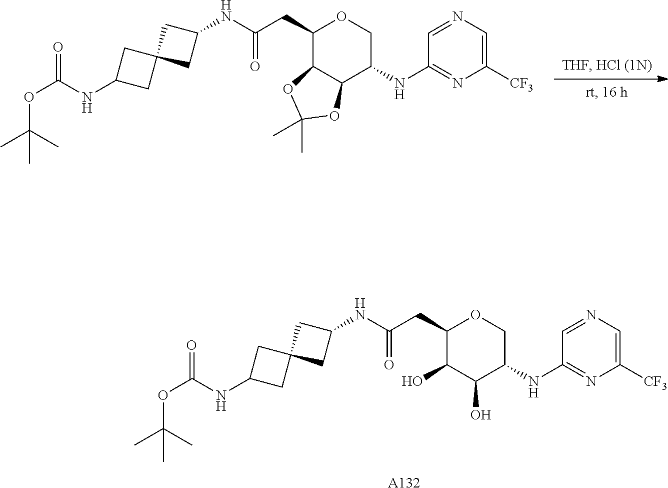

To a solution o tert-butyl (6-(2-((3a,4R,7S,7aR)-2,2-dimethyl-7-((6-trifluoromethyl)pyrazin-2-yl)amino)tetrahydro-4H-[1,3]dioxolo[4,5-c]pyran-4-yl)acetamido) spiro[3.3]heptan-2-yl)carbamate (50 mg, 0.08 mmol) in THF (5 mL) was added HCl (0.05 mL, 1N) at an ambient temperature, and then the mixture was stirred at an ambient temperature for 16 h. After completion, the pH of the mixture was adjusted to 7 by NH₃·H₂O, then the mixture was concentrated in vacuum. The residue was purified by Prep-TLC (10% MeOH in DCM) to give tert-butyl (6-(2-((2R,3R,4R,5S)-3,4-dihydroxy-5-((6-(trifluoromethyl)pyrazin-2-yl)amino)tetrahydro-2H-pyran-2-yl)acetamido)spiro [3.3]heptan-2-yl)carbamate as white solid (16.2 mg, yield: 35%). LC-MS (ESI) found: 546 [M+H]⁺. ¹H NMR (400 MHz, CD₃OD) δ 8.09 (s, 1H), 7.99 (s, 1H), 4.31 (td, J=10.5, 5.3 Hz, 1H), 4.14 (dd, J=15.8, 8.1 Hz, 1H), 4.05 (dd, J=10.2, 4.3 Hz, 1H), 3.88 (s, 1H) 3.81 (dd, J=8.7, 4.5 Hz, 1H), 3.76 (d, J=3.2 Hz, 1H), 3.66 (dd, J=10.5, 3.2 Hz, 1H), 3.15-3.03 (m, 1H), 2.58 (dd, J=14.6, 8.7 Hz, 1H), 2.47-2.33 (m, 3H), 2.24 (td, J=12.1, 7.1 Hz, 2H) 1.93 (ddd, J=20.2, 16.7, 10.4 Hz, 4H), 1.41 (s, 9H).

### Preparation of (2R,3R,4R,5R,6R)-6-azido-2-(hydroxymethyl)-5-((4-(trifluoromethyl)pyrimidin-2-yl)amino)tetrahydro-2H-pyran-3,4-diol and (2R,3R,4R,5R,6R)-5-amino-6-azido-2-(hydroxymethyl)tetrahydro-2H-pyran-3,4-diol (A133)

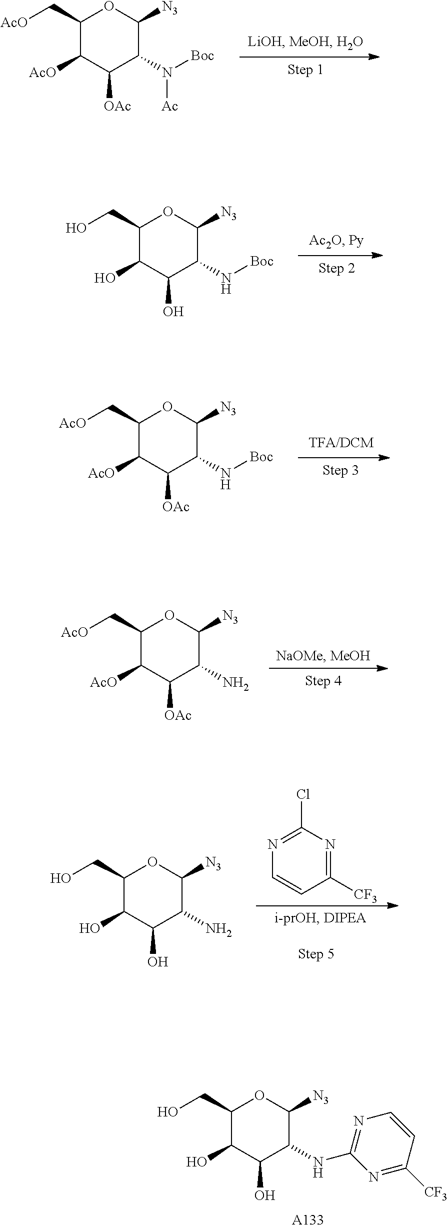

Step 1: To a solution of (2R,3R,4R,5R,6R)-2-(acetoxymethyl)-6-azido-5-(N-(tert-butoxycarbonyl)acetamido)tetrahydro-2H-pyran-3,4-diyl diacetate (800 mg, 1.68 mmol) in MeOH (5 mL) was added LiOH (533 mg, 12 mmol) in H₂O (1 mL). The mixture was stirred at an ambient temperature overnight, then concentrated to give crude tert-butyl ((2R,3R,4R,5R,6R)-2-azido-4,5-dihydroxy-6-(hydroxymethyl)tetrahydro-2H-pyran-3-yl)carbamate (400 mg, 78% yield) as white solid. LC-MS (ESI) found: 305 [M+H]⁺.
Step 2: A solution of crude tert-butyl ((2R,3R,4R,5R,6R)-2-azido-4,5-dihydroxy-6-(hydroxymethyl)tetrahydro-2H-pyran-3-yl)carbamate (400 mg, 1.31 mmol) and Ac₂O (5 mL) in Py (1 mL) was stirred at an ambient temperature overnight. The mixture was concentrated and the residue was purified by flash to give (2R,3R,4R,5R,6R)-2-(acetoxymethyl)-6-azido-5-((tert-butoxycarbonyl)amino)tetrahydro-2H-pyran-3,4-diyl diacetate (400 mg, 71% yield) as colorless oil. LC-MS (ESI) found: 453 [M+Na]⁺.
Step 3: To a solution of (2R,3R,4R,5R,6R)-2-(acetoxymethyl)-6-azido-5-((tert-butoxycarbonyl)amino)tetrahydro-2H-pyran-3,4-diyl diacetate (400 mg, 0.93 mmol) in DCM (3 mL) was added TFA (1 mL). The mixture was stirred at an ambient temperature overnight, then concentrated to give (2R,3R,4R,5R,6R)-2-(acetoxymethyl)-5-amino-6-azidotetrahydro-2H-pyran-3,4-diyl diacetate (280 mg, 92% yield) as colorless oil. LC-MS (ESI) found: 331 [M+H]⁺.
Step 4: To a solution of (2R,3R,4R,5R,6R)-2-(acetoxymethyl)-5-amino-6-azidotetrahydro-2H-pyran-3,4-diyl diacetate (280 mg, 0.84 mmol) in MeOH (3 mL) was added NaOMe (0.1 mL, 5 N in MeOH). The mixture was stirred at an ambient temperature overnight, then concentrated. The residue was purified by flash (MeCN/H₂O, 0.5% NH₄₀H) to give (2R,3R,4R,5R,6R)-5-amino-6-azido-2-(hydroxymethyl)tetrahydro-2H-pyran-3,4-diol (140 mg, 81% yield) as colorless oil. LC-MS (ESI) found: 205 [M+H]⁺. ¹H NMR (400 MHz, CD₃OD): δ 4.94 (d, J=9.3 Hz, 1H), 3.94 (d, J=2.9 Hz, 1H), 3.85-3.68 (m, 4H), 3.11 (dd, J=10.5, 9.4 Hz, 1H).
Step 5: A solution of (2R,3R,4R,5R,6R)-5-amino-6-azido-2-(hydroxymethyl)tetrahydro-2H-pyran-3,4-diol (150 mg, 0.73 mmol), 2-chloro-4-(trifluoromethyl)pyrimidine (200 mg, 1.1 mmol) and DIPEA (188 mg, 1.5 mmol) in i-PrOH (3 mL) was stirred at 80° C. overnight. The mixture was concentrated and the residue was purified by prep-HPLC (MeCN/H₂O, 0.1% HCOOH) to give (2R,3R,4R,5R,6R)-6-azido-2-(hydroxymethyl)-5-((4-(trifluoromethyl)pyrimidin-2-yl)amino) tetrahydro-2H-pyran-3,4-diol (2.7 mg, 1% yield) as white solid. LC-MS (ESI) found: 351[M+H]⁺. ¹H NMR (400 MHz, CD₃OD): δ 8.52 (d, J=4.9 Hz, 1H), 6.91 (d, J=4.9 Hz, 1H), 4.53 (d, J=9.2 Hz, 1H), 4.37 (t, J=9.8 Hz, 1H), 3.92 (d, J=2.8 Hz, 1H), 3.83 (dd, J=11.4, 6.9 Hz, 1H), 3.76 (dd, J=11.4, 5.1 Hz, 2H), 3.66-3.61 (m, 1H).

### Preparation of 6-azido-N-{1,3-bis[2-({1-[(2R,3R,4R,5S)-3,4-dihydroxy-5-{[4-(trifluoromethyl)pyrimidin-2-yl]amino}oxan-2-yl]-2,5,8,11-tetraoxatridecan-13-yl}carbamoyl)ethoxy]-2-{[2-({1-[(2R,3R,4R,5S)-3,4-dihydroxy-5-{[4-(trifluoromethyl) pyrimidin-2-yl]amino}oxan-2-yl]-2,5,8,11-tetraoxatridecan-13-yl}carbamoyl)ethoxy]methyl}propan-2-yl}hexanamide (A134)

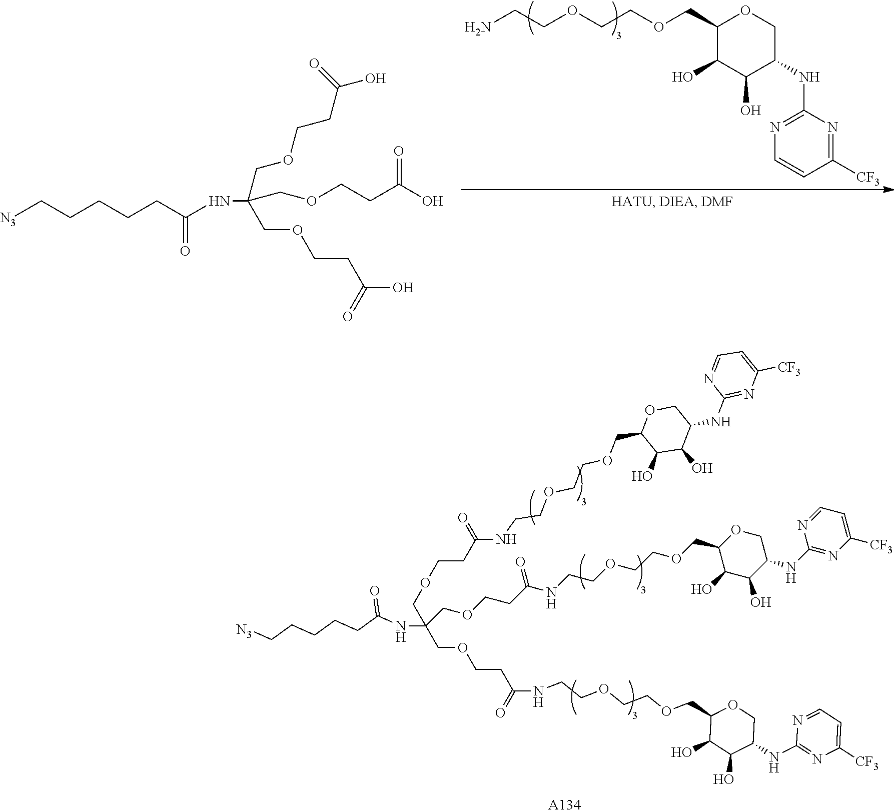

A solution of 3,3′-((2-(6-azidohexanamido)-2-((2-carboxyethoxy)methyl)propane-1,3diyl) bis(oxy))dipropionic acid (141.62 mg, 0.30 mmol), DIEA (230.28 mg, 1.79 mmol) and HATU (452.23 mg, 1.19 mmol) in DMF (5 mL) was stirred at an ambient temperature for 30 min. Then (2R,3R,4R,5S)-2-(13-amino-2,5,8,11-tetraoxatridecyl)-5-((4-(trifluoromethyl)pyrimidin-2-yl)amino)tetrahydro-2H-pyran-3,4-diol (576.0 mg, 1.19 mmol) was added, the mixture was stirred at an ambient temperature overnight. The resulting mixture was purified by flash chromatography reverse phase (C18, MeCN in H₂O from 5% to 50%) to give 6-azido-N-{1,3-bis[2-({1-[(2R,3R,4R,5S)-3,4-dihydroxy-5-{[4-(trifluoromethyl)pyrimidin-2-yl]amino}oxan-2-yl]-2,5,8,11-tetraoxatridecan-13-yl}carbamoyl) ethoxy]-2-{[2-({1-[(2R,3R,4R,5S)-3,4-dihydroxy-5-{[4-(trifluoromethyl)pyrimidin-2-yl]amino}oxan-2-yl]-2,5,8,11-tetraoxatridecan-13-yl}carbamoyl) ethoxy]methyl}propan-2-yl}hexanamide (342.5 mg, 61.43% yield) as a yellow oil. LC-MS (ESI) found: 1876 [M+H]⁺. ¹H NMR (400 MHz, MeOD) δ 8.51 (d, J=4.7 Hz, 3H), 6.89 (d, J=4.9 Hz, 3H), 4.36 (td, J=10.3, 5.2 Hz, 3H), 4.09 (dd, J=10.9, 5.1 Hz, 3H), 3.93 (d, J=3.0 Hz, 3H), 3.74-3.67 (m, 20H), 3.67-3.60 (m, 40H), 3.60-3.53 (m, 8H), 3.38 (t, J=5.5 Hz, 6H), 3.17 (t, J=10.9 Hz, 3H), 2.45 (t, J=6.1 Hz, 6H), 2.21 (t, J=7.4 Hz, 2H), 1.64-1.55 (m, 4H), 1.44-1.35 (m, 2H).

### Preparation of N-((3aS,4R,7S,7aR)-4-(azidomethyl)-2,2-dimethyltetrahydro-4H-[1,3]dioxolo[4,5-c]pyran-7-yl)-6-(trifluoromethyl)pyrazin-2-amine (A135)

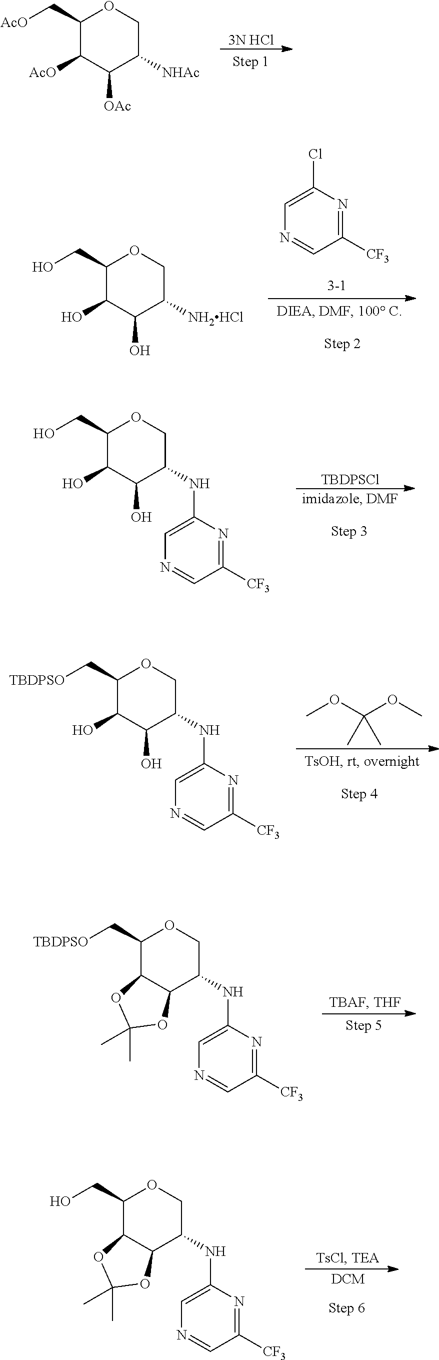

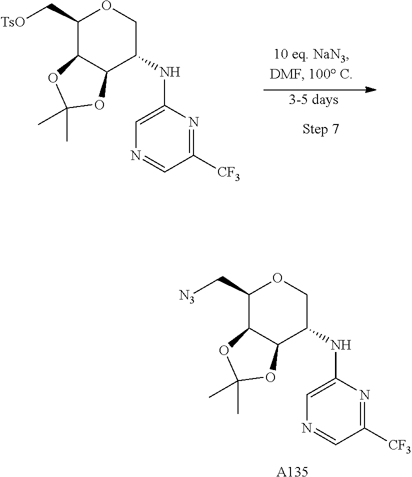 [SMILES: Cc1ccc(S(=O)(=O)OC[C@H]2OC[C@H](Nc3cncc(C(F)(F)F)n3)[C@H]3OC(C)(C)O[C@H]32)cc1 ; "3-5 days" ; CC1(C)O[C@@H]2[C@H](O1)[C@@H](Nc1cncc(C(F)(F)F)n1)CO[C@@H]2CN=[N+]=[N-] ; "A135"]

Step 1: A solution of (2R,3R,4R,5S)-5-acetamido-2-(acetoxymethyl)tetrahydro-2H-pyran-3,4-diyl diacetate (100 g, 300 mmol) in 3 M HCl (in water, 150 mL) was stirred at 100° C. for 18 h. Then the reaction was concentrated and recrystallized with isopropyl alcohol to give (2R,3R,4R,5S)-5-amino-2-(hydroxymethyl)oxane-3,4-diol (50 g, crude) as a grey solid. ¹H NMR (400 MHz, CD₃OD) δ 3.94 (dd, J=10.2, 4.1 Hz, 1H), 3.83-3.78 (m, 1H), 3.68 (ddd, J=16.4, 11.4, 6.0 Hz, 2H), 3.40 (ddd, J=6.9, 5.0, 0.9 Hz, 1H), 3.28 (d, J=3.2 Hz, 1H), 3.07 (ddd, J=15.9, 15.2, 7.6 Hz, 2H).
Step 2: To a solution of (2R,3R,4R,5S)-5-amino-2-(hydroxymethyl)oxane-3,4-diol (50 g, 306.4 mmol) and 2-chloro-6-(trifluoromethyl)pyrazine (87.8 g, 480.1 mmol) in DMF (200 mL) was added DIEA (101.3 mL, 612.8 mmol) at an ambient temperature. The reaction was stirred at 100° C. for 18 h. LCMS showed the desired mass was detected. The reaction was concentrated and the residue was purified by silica gel chromatography eluted with DCM/MeOH=10/1 to give (2R,3R,4R,5S)-2-(hydroxymethyl)-5-{[6-(trifluoromethyl)pyrazin-2-yl]amino}oxane-3,4-diol (40 g, 129.4 mmol, 42.2% yield) as yellow solid. LC-MS (ESI) found: 310.1 [M+H]⁺
Step 3: To solution of (2R,3R,4R,5S)-2-(hydroxymethyl)-5-{[6-(trifluoromethyl)pyrazin-2-yl]amino}oxane-3,4-diol (34 g, 109.9 mmol) in DMF (100 mL) was added imidazole (22.5 g, 329.8 mmol) and TBDPSCl (60.25 g, 219.9 mmol) at an ambient temperature. The reaction was stirred at an ambient temperature for 18 h. LCMS showed the desired mass was detected. The reaction was quenched with water and extracted with ethyl acetate. The organic layer was dried over Na₂SO₄, filtered and concentrated and purified by silica gel with petroleum ether/ethyl acetate=3/1 to give (2R,3R,4R,5S)-2-{[(tert-butyldiphenylsilyl)oxy]methyl}-5-{[6-(trifluoromethyl)pyrazin-2-yl]amino}oxane-3,4-diol (50 g, 91.3 mmol, 83.0% yield) as yellow oil. LC-MS (ESI) found: 548.3 [M+H]+H NMR (400 MHz, CDCl₃) δ 8.15 (s, 1H), 8.11 (s, 1H), 7.69 (ddd, J=13.1, 7.9, 1.5 Hz, 4H), 7.42 (ddd, J=14.5, 7.2, 3.2 Hz, 6H), 5.11 (d, J=6.1 Hz, 1H) 4.31 (dd, J=11.0, 5.1 Hz, 1H), 4.22-4.15 (m, 2H), 3.91 (dd, J=5.6, 2.8 Hz, 2H), 3.59 (d, J=8.6 Hz, 1H), 3.47-3.39 (m, 1H), 3.20 (s, 1H), 3.08 (t, J=10.8 Hz, 2H), 1.08 (s, 9H).
Step 4: A solution of (2R,3R,4R,5S)-2-{[(tert-butyldiphenylsilyl)oxy]methyl}-5-{[6-(trifluoromethyl)pyrazin-2-yl]amino}oxane-3,4-diol (30 g, 54.8 mmol) and TsOH (0.94 g, 5.5 mmol) in 2,2-dimethoxypropane (50 mL, 403.3 mmol) was stirred at an ambient temperature for 18 h. LCMS showed the desired mass was detected. The reaction was concentrated and purified by silica gel chromatography eluted with petroleum ether/ethyl acetate=3/1 to give N-[(3aR,4R,7S,7aR)-4-{[(tert-butyldiphenylsilyl)oxy]methyl}-2,2-dimethyl-hexahydro-[1,3]dioxolo[4,5-c]pyran-7-yl]-6-(trifluoromethyl)pyrazin-2-amine (28 g, 47.6 mmol, 87% yield) as yellow oil. LC-MS (ESI) found: 588.3 [M+H]⁺
Step 5: To a solution of N-[(3aR,4R,7S,7aR)-4-{[(tert-butyldiphenylsilyl)oxy]methyl}-2,2-dimethyl-hexahydro-[1,3]dioxolo[4,5-c]pyran-7-yl]-6-(trifluoromethyl)pyrazin-2-amine (28 g, 47.6 mmol) in THF (150 mL) was added TBAF (18.7 g, 71.5 mmol) at an ambient temperature. The reaction was stirred at an ambient temperature for 18 h. LCMS showed the desired mass was detected. The reaction was concentrated and purified by silica gel chromatography eluted with DCM/MeOH=10/1 to give [(3aR,4R,7S,7aR)-2,2-dimethyl-7-{[6-(trifluoromethyl)pyrazin-2-yl]amino}-hexahydro-[1,3]dioxolo[4,5-c]pyran-4-yl]methanol (12 g, 34.4 mmol, 72.1% yield) as yellow oil. LC-MS (ESI) found: 350.1 [M+H]⁺.
Step 6: To a solution of [(3aR,4R,7S,7aR)-2,2-dimethyl-7-{[6-(trifluoromethyl)pyrazin-2-yl]amino}-hexahydro-[1,3]dioxolo[4,5-c]pyran-4-yl]methanol (8 g, 22.9 mmol) in DCM (100 mL) was added Et₃N (9.5 mL, 68.7 mmol) and TsCl (8.7 g, 45.8 mmol) and the reaction was stirred at an ambient temperature for 18h. LC-MS showed the desired mass was detected. The reaction was purified by silica gel chromatography eluted with petroleum ether/ethyl acetate=1/1 to give [(3aR,4R,7S,7aR)-2,2-dimethyl-7-{[6-(trifluoromethyl)pyrazin-2-yl]amino}-hexahydro-[1,3]dioxolo[4,5-c]pyran-4-yl]methyl 4-methylbenzene-1-sulfonate (10 g, 19.8 mmol, 86.7% yield) as yellow oil. LC-MS (ESI) found: 504.1[M+H]⁺.
Step 7: To a solution of [(3aR,4R,7S,7aR)-2,2-dimethyl-7-{[6-(trifluoromethyl)pyrazin-2-yl]amino}-hexahydro-[1,3]dioxolo[4,5-c]pyran-4-yl]methyl 4-methylbenzene-1-sulfonate (10 g, 19.8 mmol) in DMF (50 mL) was added sodium azide (12.9 g, 198.6 mmol) at an ambient temperature. The reaction was stirred at 100° C. for 3d. The reaction was extracted with EA and water. The organic layer was dried over Na₂SO₄, filtered and concentrated to give crude mixture. The crude product was purified by silica gel chromatography eluted with petroleum ether/ethyl acetate=1/1 to give N-[(3aS,4R,7S,7aR)-4-(azidomethyl)-2,2-dimethyl-hexahydro-[1,3]dioxolo[4,5-c]pyran-7-yl]-6-(trifluoromethyl) pyrazin-2-amine (5 g, 13.4 mmol, 67.3% yield) as yellow oil. LC-MS (ESI) found: 375.2[M+H]⁺. ¹H NMR (400 MHz, DMSO-d₆) δ 8.21 (s, 1H) 8.11 (s, 1H), 7.81 (d, J=7.6 Hz, 1H), 4.05-4.25 (m, 3H), 3.94-3.96 (m, 1H), 3.83-3.86 (m, 1H) 3.54 (d, J=8.8 Hz, 1H), 3.35-3.45 (m, 1H), 3.15 (t, J=10.8 Hz, 1H), 1.43 (s, 3H), 1.27 (s, 3H).

### Preparation of tert-butyl 1-amino-12-(2-(2-(2-(2-aminoethoxy)ethoxy)ethoxy)ethyl) 3,6,9,15,18,21-hexaoxa-12-azatetracosan-24-oate

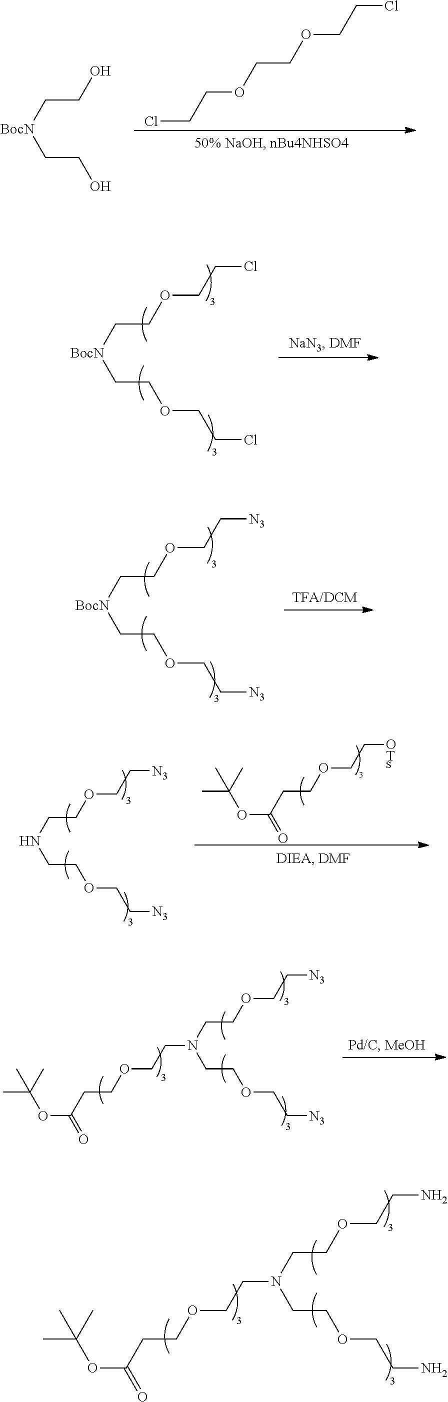

### Step 1

To a solution of tert-butyl bis(2-hydroxyethyl)carbamate (1.00 eq) in 1,2-bis(2-chloroethoxy)ethane (15.0 eq) were added n-Bu₄NHSO₄ (1.00 eq) and NaOH (50%, 1 mL) at 0° C. The mixture was stirred at an ambient temperature for 2 hours. Then the reaction was quenched with sat. NH₄Cl (aq.) dropwise. The mixture was concentrated and purified by silica gel chromatography to afford tert-butyl bis(2-(2-(2-(2-chloroethoxy)ethoxy)ethoxy)ethyl)carbamate as a colorless oil. Chemical Formula: C₂₁H₄₁Cl₂NO₈, LCMS found: [M+H]⁺=506.

### Step 2

To a solution of tert-butyl bis(2-(2-(2-(2-chloroethoxy)ethoxy)ethoxy)ethyl)carbamate (1.00 eq) in DMF was added NaN₃ (8.00 eq) at an ambient temperature. The mixture was stirred at 100° C. overnight. Then the reaction was quenched with brine and extracted with ethyl acetate. The organic layer was concentrated and purified by silica gel chromatography to afford tert-butyl bis(2-(2-(2-(2-azidoethoxy)ethoxy)ethoxy)ethyl)carbamate as a colorless oil. Chemical Formula: C₂₁H₄₁N₇O₈, LCMS found: [M+H]⁺=520.

### Step 3

To a solution of tert-butyl bis(2-(2-(2-(2-azidoethoxy)ethoxy)ethoxy)ethyl)carbamate (1.00 eq) in DCM was added TFA at 0° C. The mixture was stirred at an ambient temperature overnight. Then the mixture was concentrated to afford bis(2-(2-(2-(2-azidoethoxy)ethoxy)ethoxy)ethyl)amine as a colorless oil. Chemical Formula: C₁₆H₃₃N₇O₆, LC-MS found: [M+H]⁺=420.

### Step 4

To a solution of bis(2-(2-(2-(2-azidoethoxy)ethoxy)ethoxy)ethyl)amine (1.00 eq) in DMF were added tert-butyl 3-(2-(2-(2-(tosyloxy)ethoxy)ethoxy)ethoxy)propanoate (1.50 eq) and K₂CO₃ (1.50 eq) at an ambient temperature. The mixture was stirred at 100° C. overnight. Then the mixture was concentrated and purified by silica gel chromatography to afford tert-butyl 1-azido-12-(2-(2-(2-(2-azidoethoxy)ethoxy)ethoxy)ethyl)-3,6,9,15,18,21-hexaoxa-12-azatetracosan-24-oate as a brown oil. Chemical Formula: C₂₉H₅₇N₇O₁₁, LC-MS found: [M+H]⁺=680.

### Step 5

To a solution of tert-butyl 1-azido-12-(2-(2-(2-(2-azidoethoxy)ethoxy)ethoxy)ethyl) 3,6,9,15,18,21-hexaoxa-12-azatetracosan-24-oate (1.00 eq) in MeOH was added Pd/C (10% purity) at an ambient temperature. The mixture was stirred at an ambient temperature under a H2 balloon overnight. The mixture was filtered through a Celite pad, and the filtrate was concentrated to afford tert-butyl 1-amino-12-(2-(2-(2-(2-aminoethoxy)ethoxy)ethoxy)ethyl)-3,6,9,15,18,21-hexaoxa-12-azatetracosan-24-oate as a brown oil. Chemical Formula: C₂₉H₆₁N₃O₁₁, LC-MS found: [M+H]⁺=628. ¹H NMR (400 MHz, CD₃OD): δ 3.79-3.50 (m, 38H), 2.82 (dt, J=11.0, 5.2 Hz, 8H), 2.48 (t, J=6.1 Hz, 2H), 1.45 (s, 9H).

### Preparation of N-((2S,3S,4R,5R,6R)-4,5-dihydroxy-6-(hydroxymethyl)-2-phenyltetrahydro-2H-pyran-3-yl)acetamide (A136)

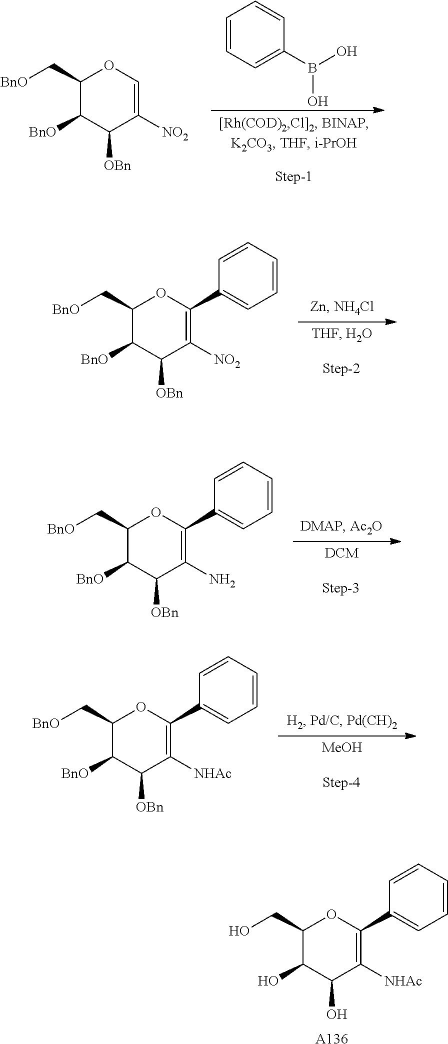

### Step 1

A solution of BINAP (0.05 eq) and [Rh(COD)₂Cl]₂ (0.05 eq) in THF was stirred at an ambient temperature under nitrogen for 30 minutes, then (2R,3R,4R)-3,4-bis(benzyloxy)-2-((benzyloxy)methyl)-5-nitro-3,4-dihydro-2H-pyran (1.00 eq), phenylboronic acid (3.00 eq) and K₂CO₃ (2.00 eq) in THF and i-PrOH was added. The resulting mixture was stirred at 100° C. overnight under nitrogen atmosphere. The mixture was concentrated and purified by flash to afford (2R,3R,4R,5R,6S)-3,4-bis(benzyloxy)-2-((benzyloxy)methyl)-5-nitro-6-phenyltetrahydro-2H-pyran as a colorless oil. Chemical Formula: C₃₃H₃₃NO₆, LCMS found: [M+H]⁺=540.

### Step 2

A solution of (2R,3R,4R,5R,6S)-3,4-bis(benzyloxy)-2-((benzyloxy)methyl)-5-nitro-6-phenyltetrahydro-2H-pyran (1.00 eq) and Zn (10.0 eq) and NH₄Cl (10.0 eq) in THF and H₂O was stirred at 70° C. overnight. The mixture was filtered through a Celite pad, and the filtrate was concentrated to afford (2S,3R,4R,5R,6R)-4,5-bis(benzyloxy)-6-((benzyloxy)methyl)-2-phenyltetrahydro-2H-pyran-3-amine as a colorless oil. Chemical Formula: C₃₃H₃₅NO₄, LC-MS found: [M+H]⁺=510.

### Step 3

To a solution of (2S,3R,4R,5R,6R)-4,5-bis(benzyloxy)-6-((benzyloxy)methyl)-2-phenyltetrahydro-2H-pyran-3-amine (1.00 eq) in DCM was added DMAP (0.10 eq) and Ac₂O (10.0 eq) at 0° C. The mixture was stirred at an ambient temperature overnight. Then the mixture was concentrated and the residue was purified by silica gel chromatography to afford N-((2S,3R,4R,5R,6R)-4,5-bis(benzyloxy)-6-((benzyloxy)methyl)-2-phenyltetrahydro-2H-pyran-3-yl)acetamide as a white solid. Chemical Formula: C₁₆H₃₇NO₅, LCMS found: [M+H]⁺=552.

### Step 4

To a solution of N-((2S,3R,4R,5R,6R)-4,5-bis(benzyloxy)-6-((benzyloxy)methyl)-2-phenyltetrahydro-2H-pyran-3-yl)acetamide (1.00 eq) in MeOH were added Pd/C (10% purity) and Pd(OH)₂ (10% purity) at an ambient temperature. The mixture was stirred at an ambient temperature under a H2 balloon overnight. The mixture was filtered through a Celite pad, and the filtrate was concentrated, the residue was purified by reverse phase to afford tert-butyl 1-amino-12-(2-(2-(2-(2-aminoethoxy)ethoxy)ethoxy)ethyl)-3,6,9,15,18,21-hexaoxa-12-azatetracosan-24-oate as a white solid. Chemical Formula: C₁₄H₁₉NO₅, LC-MS found: [M+H]⁺=282. ¹H NMR (400 MHz, CD₃OD): δ 7.37 (d, J=7.3 Hz, 2H), 7.26 (dd, J=10.1, 4.7 Hz, 2H), 7.19 (dd, J=8.4, 6.1 Hz, 1H), 4.66 (s, 1H), 4.48-4.42 (m, 1H), 3.96-3.93 (m, 2H), 3.89 (dd, J=11.4, 6.8 Hz, 1H) 3.80 (dd, J=11.4, 5.0 Hz, 1H), 3.67-3.61 (m, 1H), 1.72 (s, 3H).

### Preparation of N-((2S,3R,4R,5R,6R)-4,5-dihydroxy-6-(hydroxymethyl)-2-phenyltetrahydro-2H-pyran-3-yl)acetamide (A137)

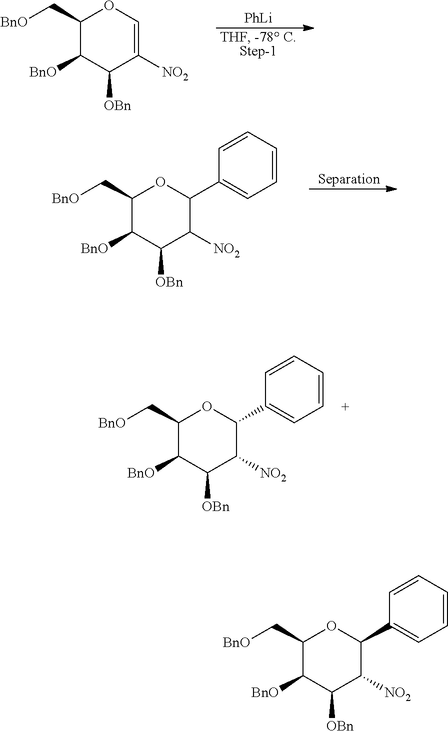

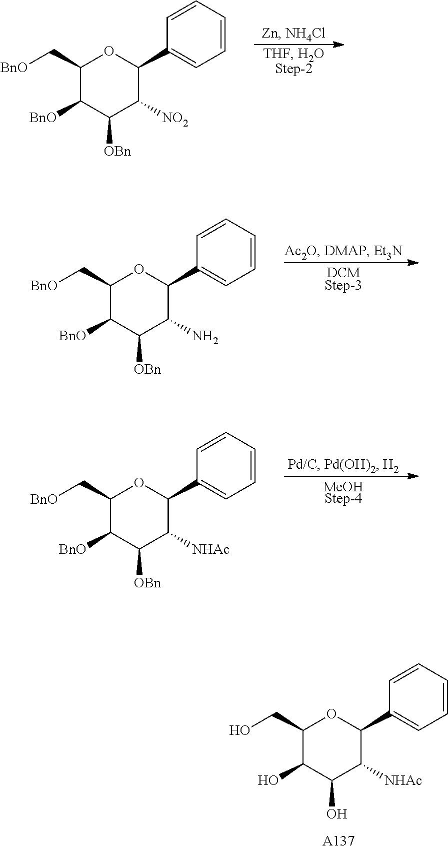

To a solution of (2R,3R,4R)-3,4-bis(benzyloxy)-2-((benzyloxy)methyl)-5-nitro-3,4-dihydro-2H-pyran (1.00 eq) in THF was added PhLi (1.50 eq) at −78° C. The mixture was stirred at −78° C. for 2 h. The mixture was quenched with NH₄Cl (aq.), then concentrated under reduced pressure, the residue was purified by flash to afford (2R,3R,4R,5S,6R)-3,4-bis(benzyloxy)-2-((benzyloxy)methyl)-5-nitro-6-phenyltetrahydro-2H-pyran and (2R,3R,4R,5S,6S)-3,4-bis(benzyloxy)-2-((benzyloxy)methyl)-5-nitro-6-phenyltetrahydro-2H-pyran as colorless oil. Chemical Formula: C₃₃H₃₃NO₆, LCMS found: [M+H]⁺=540.

### Step 2

A solution of (2R,3R,4R,5S,6S)-3,4-bis(benzyloxy)-2-((benzyloxy)methyl)-5-nitro-6-phenyltetrahydro-2H-pyran (1.00 eq) and Zn (10.0 eq) and NH₄Cl (10.0 eq) in THF and H₂O was stirred at 70° C. overnight. The mixture was filtered through a Celite pad, the filtrate was concentrated to afford (2S,3S,4R,5R,6R)-4,5-bis(benzyloxy)-6-((benzyloxy)methyl)-2-phenyltetrahydro-2H-pyran-3-amine as a colorless oil. Chemical Formula: C₃₃H₃₅NO₄, LCMS found: [M+H]⁺=510.

### Step 3

To a solution of (2S,3S,4R,5R,6R)-4,5-bis(benzyloxy)-6-((benzyloxy)methyl)-2-phenyltetrahydro-2H-pyran-3-amine (1.00 eq) in DCM was added DMAP (0.10 eq) and Ac₂O (2.00 eq) at 0° C. The mixture was stirred at an ambient temperature overnight. Then the mixture was concentrated and purified by flash to afford N-((2S,3R,4R,5R,6R)-4,5-bis(benzyloxy)-6-((benzyloxy)methyl)-2-phenyltetrahydro-2H-pyran-3-yl)acetamide as a white solid. Chemical Formula: C₁₆H₃₇NO₅, LCMS found: [M+H]⁺=552.

### Step 4

To a solution of N-((2S,3R,4R,5R,6R)-4,5-bis(benzyloxy)-6-((benzyloxy)methyl)-2-phenyltetrahydro-2H-pyran-3-yl)acetamide (1.00 eq) in MeOH were added Pd/C (10% purity) and Pd(OH)₂ (10% purity) at an ambient temperature. The mixture was stirred at an ambient temperature under a H2 balloon overnight. The mixture was filtered through a Celite pad, and the filtrate was concentrated to afford N-((2S,3R,4R,5R,6R)-4,5-dihydroxy-6-(hydroxymethyl)-2-phenyltetrahydro-2H-pyran-3-yl)acetamide as a white solid. Chemical Formula: C₁₄H₁₉NO₅, LCMS found: [M+H]⁺=282. ¹H NMR (400 MHz, CD₃OD): δ 7.48 (d, J=7.6 Hz, 2H), 7.34 (t, J=7.5 Hz, 2H), 7.26 (t, J=9.5 Hz, 1H), 4.90 (d, J=5.5 Hz, 1H), 4.53-4.50 (m, 1H), 4.05 (dd, J=13.1, 8.7 Hz, 1H), 3.90 (t, J=3.2 Hz, 1H), 3.76 (dt, J=7.3, 4.0 Hz, 3H), 1.90 (s, 3H).

### Preparation of (2R,3R,4R,5S)-5-((6-bromopyrazin-2-yl)amino)-2-(hydroxymethyl) tetrahydro-2H-pyran-3,4-diol (A138)

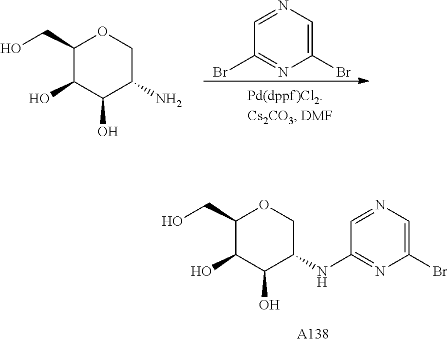 [SMILES: N[C@H]1CO[C@H](CO)[C@H](O)[C@@H]1O ; Brc1cncc(Br)n1 ; OC[C@H]1OC[C@H](Nc2cncc(Br)n2)[C@@H](O)[C@H]1O ; "A138"]

To a solution of (2R,3R,4R,5S)-5-amino-2-(hydroxymethyl)tetrahydro-2H-pyran-3,4-diol (1.00 eq) in DMF (2.5 ml) was added 2,6-dibromopyrazine (1.50 eq), Pd(dppf)C12 (0.20 eq) and Cs₂CO₃ (2.00 eq), the mixture was degassed and purged with nitrogen several times, then stirred at 100° C. for 6 hours under nitrogen atmosphere. The mixture was filtered, the filtrate was concentrated under reduced pressure, the residue was purified by prep-HPLC to afford (2R,3R,4R,5S)-5-((6-bromopyrazin-2-yl)amino)-2-(hydroxymethyl)tetrahydro-2H-pyran-3,4-diol (compound A138) as a white solid. Chemical Formula: C₁₀H₁₄BrN₃O₄, LCMS found: [M+H]⁺=320, 322. ¹H NMR (400 MHz, CD₃OD): δ 7.82 (s, 1H), 7.72 (s, 1H), 4.29 (td, J=10.8, 5.2 Hz, 1H), 4.09 (dd, J=10.8, 5.2 Hz, 1H), 3.91 (d, J=2.8 Hz, 1H), 3.76 (dd, J=11.2, 7.2 Hz, 1H), 3.68 (dd, J=11.2, 5.2 Hz, 1H) 3.60 (dd, J=10.4, 3.2 Hz, 1H), 3.49-3.42 (m, 1H), 3.11 (t, J=10.8 Hz, 1H).

### Preparation of 2-(((3S,4R,5R,6R)-4,5-dihydroxy-6-(hydroxymethyl)tetrahydro-2H-pyran-3-yl)amino)-6-(trifluoromethyl)isonicotinonitrile (A139)

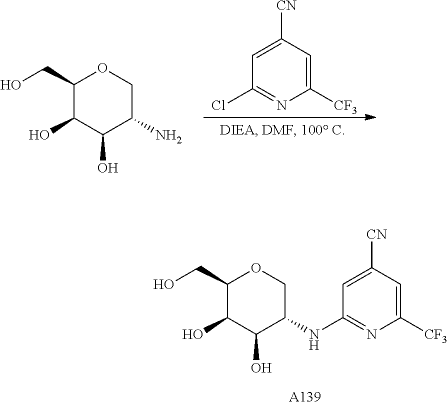 [SMILES: N[C@H]1CO[C@H](CO)[C@H](O)[C@@H]1O ; N#Cc1cc(Cl)nc(C(F)(F)F)c1 ; N#Cc1cc(N[C@H]2CO[C@H](CO)[C@H](O)[C@@H]2O)nc(C(F)(F)F)c1 ; "A139"]

The mixture of (2R,3R,4R,5S)-5-amino-2-(hydroxymethyl)tetrahydro-2H-pyran-3,4-diol (1.00 eq), 2-chloro-6-(trifluoromethyl)isonicotinonitrile (1.20 eq) and DIEA (3.00 eq) in DMF was stirred at 100° C. for 12 hours. The mixture was concentrated under reduced pressure, the residue was purified by prep-HPLC to afford 2-(((3S,4R,5R,6R)-4,5-dihydroxy-6-(hydroxymethyl)tetrahydro-2H-pyran-3-yl)amino)-6-(trifluoromethyl)isonicotinonitrile (A139) as a light yellow solid. Chemical Formula: C₁₃H₁₄F₃N₃O₄. LCMS found: [M+H]⁺=334. ¹H NMR (400 MHz, CD₃OD) δ 7.12 (d, J 1.2 Hz, 1H), 7.05 (d, J 1.2 Hz, 1H), 4.41-4.25 (m, 1H), 4.16 (dd, J=11.2, 5.2 Hz, 1H), 3.93 (d, J=3.2 Hz, 1H), 3.78 (dd, J=11.2, 7.2 Hz, 1H), 3.70 (dd, J=11.2, 5.2 Hz, 1H), 3.64 (dd, J=10.8, 3.2 Hz, 1H), 3.49-3.44 (m, 1H), 3.13 (t, J=10.8 Hz, 1H).

### Preparation of (2R,3R,4S,5S)-2-(hydroxymethyl)-5-((4-(trifluoromethyl)pyrimidin-2-yl)oxy)tetrahydro-2H-pyran-3,4-diol (A140)

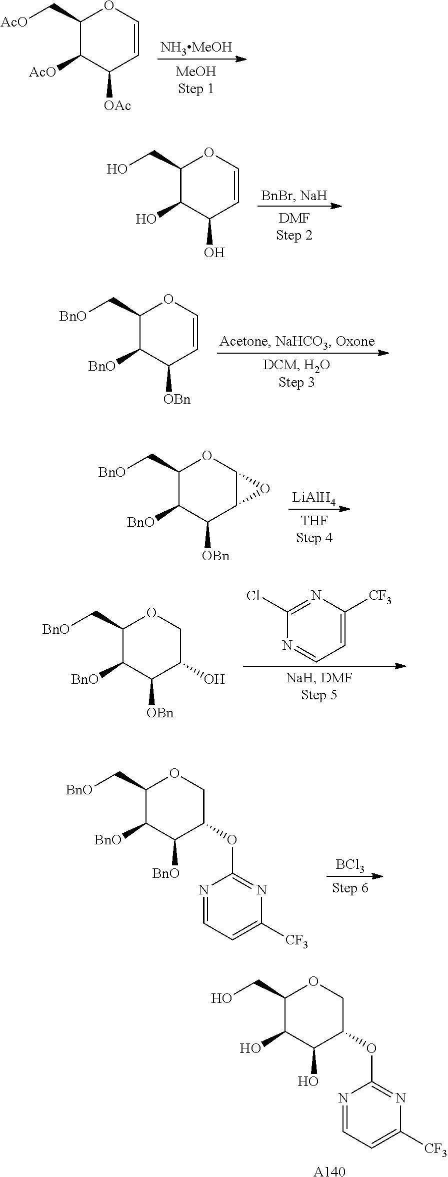

Step 1: To a solution of (2R,3R,4R)-2-(acetoxymethyl)-3,4-dihydro-2H-pyran-3,4-diyl diacetate (1.00 eq) in MeOH (50 mL) was added NH₃·MeOH (7M, 2.00 eq) at 25° C. The reaction was stirred at 25° C. for 16 h. LCMS showed the reaction was completed. The reaction was concentrated under reduced pressure to give (2R,3R,4R)-2-(hydroxymethyl)-3,4-dihydro-2H-pyran-3,4-diol (crude) as brown solid. LCMS found: [M+H]⁺=147.1.
Step 2: To a solution of (2R,3R,4R)-2-(hydroxymethyl)-3,4-dihydro-2H-pyran-3,4-diol (1.00 eq) in DMF (50 mL) was added NaH (4.00 eq) at 0° C., the mixture was stirred at 0° C. for 1 h. Then BnBr (4.00 eq) was added at 0° C. and the resulting mixture was stirred at 0° C. for 3 h. TLC (PE/EA=2/1) showed the starting material was consumed completely, one major spot was detected. The reaction was quenched with saturated solution of NH₄Cl, then extracted with ethyl acetate, the organic layer was dried over Na₂SO₄, filtered and concentrated under reduced pressure, the residue was purified by silica gel chromatography (PE/EA=10/1 to 3/1) to give (2R,3R,4R)-3,4-bis(benzyloxy)-2-((benzyloxy)methyl)-3,4-dihydro-2H-pyran as colorless oil. LCMS found: [M+Na]⁺=439.1.
Step 3: To a solution of (2R,3R,4R)-3,4-bis(benzyloxy)-2-((benzyloxy)methyl)-3,4-dihydro-2H-pyran (1.00 eq) in DCM (50 mL) was added acetone (5 mL) and saturated solution of NaHCO₃ (100 mL) at 0° C., then the solution of Oxone (2.00 eq) in H₂O (60 mL) was added dropwise over 20 min. The mixture was stirred at 0° C. for 30 min and then stirred at 25° C. for an additional 6 h. The organic layer was separated and the aqueous phase was extracted with DCM. The combined organic layers were dried over Na₂SO₄, filtered and concentrated to give (1S,3R,4S,5S,6R)-4,5-bis(benzyloxy)-3-((benzyloxy)methyl)-2,7-dioxabicyclo[4.1.0]heptane (crude) as colorless oil. LCMS found: [M+Na]=455.2
Step 4: To a solution of (1S,3R,4S,5S,6R)-4,5-bis(benzyloxy)-3-((benzyloxy)methyl)-2,7-dioxabicyclo[4.1.0]heptane (1.00 eq) in THF (40 mL) was added LiAlH₄ (1.00 eq) 0° C. The reaction was stirred at 25° C. for 18 h. The reaction was quenched with water and NaOH (15%), then filtered, the filtrate was concentrated under reduced pressure. The residue was purified by silica gel chromatography (PE/EA=10/1 to 3/1) to give (3S,4R,5S,6R)-4,5-bis(benzyloxy)-6-((benzyloxy)methyl)tetrahydro-2H-pyran-3-ol as colorless oil. LCMS found: [M+H]⁺=435.2
Step 5: To a solution of (3S,4R,5S,6R)-4,5-bis(benzyloxy)-6-((benzyloxy)methyl)tetrahydro-2H-pyran-3-ol (1.00 eq) in DMF (10 mL) was added sodium hydride (2.00 eq, 60% purity) slowly at 0° C., the resulting mixture was stirred at 0° C. for 20 min, then 2-chloro-4-(trifluoromethyl)pyrimidine (1.50 eq) was added, the resulting mixture was stirred at 25° C. for 16 h. TLC (PE/EA=3/1) showed the starting material was consumed completely, one major spot was observed. The reaction was quenched with saturated solution of NH₄Cl and extracted with ethyl acetate, the organic layer was dried over Na₂SO₄, filtered and concentrated under reduced pressure, the residue was purified by silica gel chromatography (PE/EA=20/1 to 3/1) to give 2-(((3S,4R,5S,6R)-4,5-bis(benzyloxy)-6-((benzyloxy)methyl)tetrahydro-2H-pyran-3-yl)oxy)-4-(trifluoromethyl)pyrimidine as colorless oil. LCMS found: [M+H]⁺=581.2
Step 6: To a solution of 2-(((3S,4R,5S,6R)-4,5-bis(benzyloxy)-6-((benzyloxy)methyl)tetrahydro-2H-pyran-3-yl)oxy)-4-(trifluoromethyl)pyrimidine (1.00 eq) in DCM (10 mL) was added BCl₃ (10.0 eq) at −20° C. The reaction was stirred at 25° C. for 12 h. The mixture was quenched with MeOH, then concentrated under reduced pressure, the residue was purified by prep-HPLC to give (2R,3R,4S,5S)-2-(hydroxymethyl)-5-((4-(trifluoromethyl)pyrimidin-2-yl)oxy)tetrahydro-2H-pyran-3,4-diol (A140) as white solid. LCMS found: [M+H]⁺=311.4. ¹H NMR (400 MHz, CD₃OD) δ 8.86 (d, J=4.8 Hz, 1H), 7.47 (d, J=4.8 Hz, 1H), 5.42 (td, J=10.0, 5.6 Hz, 1H), 4.28 (dd, J=10.8, 5.2 Hz, 1H), 3.99 (d, J=2.4 Hz, 1H), 3.87 (dd, J=10.0, 3.6 Hz, 1H), 3.77 (dd, J=11.2, 7.2 Hz, 1H), 3.69 (dd, J=11.2, 4.8 Hz, 1H), 3.52 (ddd, J=7.2, 5.2, 1.2 Hz, 1H), 3.30 (t, J=10.8 Hz, 1H).

### Preparation of (2R,3R,4S,5S)-2-(hydroxymethyl)-5-((6-(trifluoromethyl)pyrazin-2-yl)oxy)tetrahydro-2H-pyran-3,4-diol (A141)

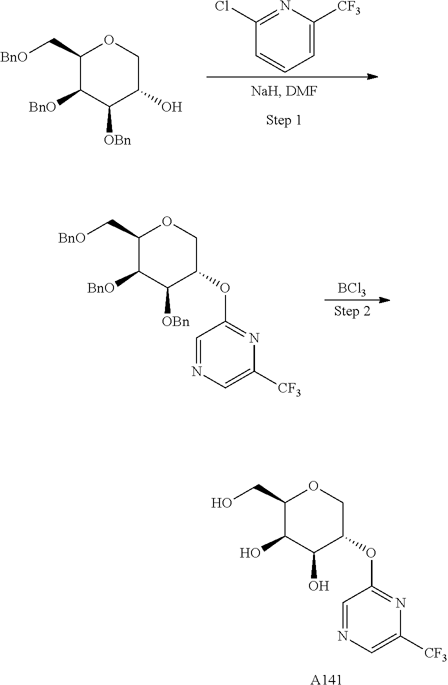 [SMILES: O[C@H]1CO[C@H](COCc2ccccc2)[C@H](OCc2ccccc2)[C@@H]1OCc1ccccc1 ; FC(F)(F)c1cccc(Cl)n1 ; FC(F)(F)c1cncc(O[C@H]2CO[C@H](COCc3ccccc3)[C@H](OCc3ccccc3)[C@@H]2OCc2ccccc2)n1 ; "BCl3" ; OC[C@H]1OC[C@H](Oc2cncc(C(F)(F)F)n2)[C@@H](O)[C@H]1O ; "A141"]

Step 1: To a solution of (3S,4R,5S,6R)-4,5-bis(benzyloxy)-6-((benzyloxy)methyl)tetrahydro-2H-pyran-3-ol (1.00 eq) in DMF (10 mL) was added sodium hydride (2.00 eq, 60% purity) slowly at 0° C., the resulting mixture was stirred at 0° C. for 20 min, then 2-chloro-6-(trifluoromethyl)pyrazine (1.50 eq) was added and the mixture was stirred at 25° C. for 16 h. TLC (PE/EA=3/1) showed the starting material was consumed completely, one major spot was detected. The reaction was quenched with saturated solution of NH₄Cl, then extracted with ethyl acetate, the organic layer was dried over Na₂SO₄, filtered and concentrated under reduced pressure, the residue was purified by silica gel chromatography (PE/EA=20/1 to 3/1) to give 2-(((3S,4R,5S,6R)-4,5-bis(benzyloxy) 6-((benzyloxy)methyl)tetrahydro-2H-pyran-3-yl)oxy)-6-(trifluoromethyl)pyrazine as colorless oil. LCMS found: [M+H]⁺=581.2
Step 2: To a solution of 2-(((3S,4R,5S,6R)-4,5-bis(benzyloxy)-6-((benzyloxy)methyl)tetrahydro-2H-pyran-3-yl)oxy)-6-(trifluoromethyl)pyrazine (1.00 eq) in DCM (10 mL) was added BCl₃ (10.0 eq) at −20° C. The reaction was stirred at 25° C. for 18 h. The mixture was quenched with MeOH, then concentrated and the residue was purified by prep-HPLC to give (2R,3R,4S,5S)-2-(hydroxymethyl)-5-((6-(trifluoromethyl)pyrazin-2-yl)oxy)tetrahydro-2H-pyran-3,4-diol (A141) as white solid. LCMS found: [M+H]⁺=311.1. ¹H NMR (400 MHz, CD₃OD) δ 8.56 (s, 1H), 8.49 (s, 1H), 5.39 (td, J=10.0, 5.2 Hz, 1H), 4.29 (dd, J=10.8, 5.6 Hz, 1H), 3.99 (d, J=2.8 Hz, 1H) 3.87 (dd, J=9.6, 3.6 Hz, 1H), 3.77 (dd, J=11.6, 7.2 Hz, 1H), 3.69 (dd, J=11.6, 5.2 Hz, 1H), 3.52 (ddd, J=7.2, 5.2, 1.2 Hz, 1H), 3.29 (t, J=10.8 Hz, 1H).

### Preparation of (2R,3R,4S,5S)-2-(hydroxymethyl)-5-((6-(trifluoromethyl)pyridin-2-yl)oxy)tetrahydro-2H-pyran-3,4-diol (A142)

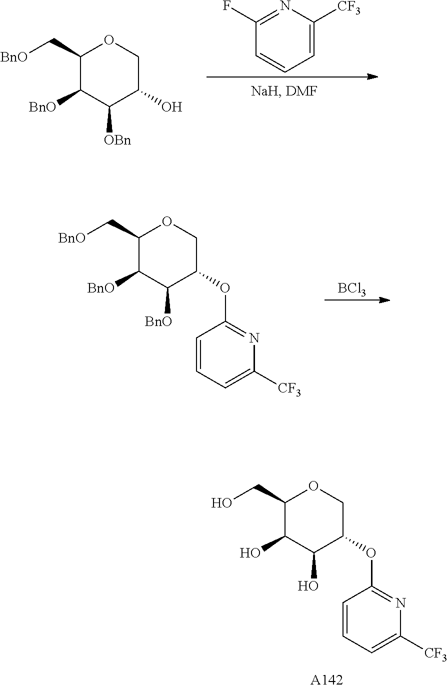 [SMILES: O[C@H]1CO[C@H](COCc2ccccc2)[C@H](OCc2ccccc2)[C@@H]1OCc1ccccc1 ; Fc1cccc(C(F)(F)F)n1 ; FC(F)(F)c1cccc(O[C@H]2CO[C@H](COCc3ccccc3)[C@H](OCc3ccccc3)[C@@H]2OCc2ccccc2)n1 ; "BCl3" ; OC[C@H]1OC[C@H](Oc2cccc(C(F)(F)F)n2)[C@@H](O)[C@H]1O ; "A142"]

Step 1: To a solution of (3S,4R,5S,6R)-4,5-bis(benzyloxy)-6-((benzyloxy)methyl)tetrahydro-2H-pyran-3-ol (1.00 eq) in DMF (10 mL) was added sodium hydride (2.50 eq, 60% purity) slowly at 0° C., the resulting mixture was stirred at 0° C. for 20 min, then 2-fluoro-6-(trifluoromethyl)pyridine (1.50 eq) was added, the resulting mixture was stirred at 25 C for 16 h. TLC (PE/EA=3/1) showed the starting material was consumed, one major spot was detected. The reaction was quenched with saturated solution of NH₄Cl, then extracted with ethyl acetate, the organic layer was dried over Na₂SO₄, filtered and concentrated under reduced pressure, the residue was purified by silica gel chromatography (PE/EA=20/1 to 3/1) to give 2-(((3S,4R,5S,6R)-4,5-bis(benzyloxy)-6-((benzyloxy)methyl)tetrahydro-2H-pyran-3-yl)oxy)-6-(trifluoromethyl)pyridine as colorless oil. LCMS found: [M+H]⁺=580.2
Step 2: To a solution of 2-(((3S,4R,5S,6R)-4,5-bis(benzyloxy)-6-((benzyloxy)methyl)tetrahydro-2H-pyran-3-yl)oxy)-6-(trifluoromethyl)pyridine (1.00 eq) in DCM (10 mL) was added BCl₃ (10.0 eq) at −20° C. The reaction was stirred at 25° C. for 16 h. The mixture was quenched with MeOH, then concentrated in vacuo, the residue was purified by prep-HPLC to give (2R,3R,4S,5S)-2-(hydroxymethyl)-5-((6-(trifluoromethyl)pyridin-2-yl)oxy)tetrahydro-2H-pyran-3,4-diol (A142) as white solid. LCMS found: [M+H]⁺=310.1. ¹H NMR (400 MHz, CD₃OD) δ 7.86 (t, J=8.0 Hz, 1H), 7.35 (d, J=8.0 Hz, 1H), 7.04 (d, J=8.0 Hz, 1H), 5.35 (td, J=10.0, 5.2 Hz, 1H), 4.35 (dd, J=10.8, 5.2 Hz, 1H), 3.98 (d, J=3.2 Hz, 1H), 3.82 (dd, J=9.6, 3.6 Hz, 1H), 3.77 (dd, J=11.2, 7.2 Hz, 1H), 3.69 (dd, J=11.2, 5.2 Hz, 1H), 3.51 (ddd, J=7.2, 5.2, 1.2 Hz, 1H), 3.22 (t, J=10.4 Hz, 1H).

### Preparation of (2R,3R,4R,5S)-2-(hydroxymethyl)-5-((6-(trifluoromethoxy)pyridin-2-yl)amino)tetrahydro-2H-pyran-3,4-diol (A143)

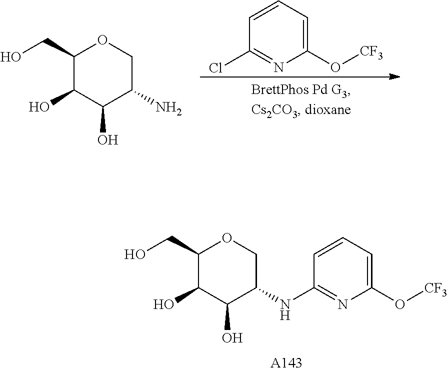 [SMILES: N[C@H]1CO[C@H](CO)[C@H](O)[C@@H]1O ; FC(F)(F)Oc1cccc(Cl)n1 ; OC[C@H]1OC[C@H](Nc2cccc(OC(F)(F)F)n2)[C@@H](O)[C@H]1O ; "A143"]

To a mixture of (2R,3R,4R,5S)-5-amino-2-(hydroxymethyl)tetrahydro-2H-pyran-3,4-diol (1.00 eq) and 2-chloro-6-(trifluoromethoxy)pyridine (1.00 eq) in dioxane was added BrettPhos Pd G3 (0.2 eq) and Cs₂CO₃ (3.00 eq), the resulting mixture was degassed and purged with nitrogen several times, then stirred at 100° C. for 12 hours under N₂ atmosphere. The mixture was concentrated under reduced pressure, the residue was purified by prep-HPLC to afford (2R,3R,4R,5S)-2-(hydroxymethyl)-5-((6-(trifluoromethoxy)pyridin-2-yl)amino)tetrahydro-2H-pyran-3,4-diol (A124) as a white solid. Chemical Formula: C₁₂H₁₅F₃N₂O₅. LCMS found: [M+H]⁺=325. ¹H NMR (400 MHz, CD₃OD) δ 7.49 (t, J=8.0 Hz, 1H), 6.46 (d, J=8.0 Hz, 1H), 6.22 (d, J=8.0 Hz, 1H), 4.22-4.12 (m, 2H), 3.92 (d, J=2.8 Hz, 1H), 3.78 (dd, J=11.2, 7.2 Hz, 1H), 3.71 (dd, J=11.2, 5.2 Hz, 1H), 3.66-3.60 (m, 1H), 3.49-3.43 (m, 1H), 3.19-3.07 (m, 1H).

### Preparation of (2R,3R,4R,5S)-5-((6-(trifluoromethoxy)pyridin-2-yl)amino)-2-(((6-(trifluoromethoxy)pyridin-2-yl)oxy)methyl) tetrahydro-2H-pyran-3,4-diol (A144)

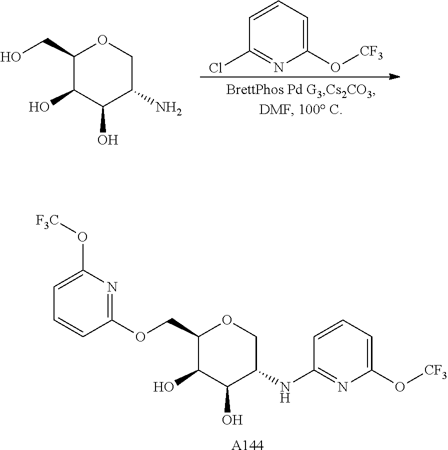

The mixture of (2R,3R,4R,5S)-5-amino-2-(hydroxymethyl)tetrahydro-2H-pyran-3,4-diol (1.00 eq), 2-chloro-6-(trifluoromethoxy)pyridine (1.00 eq), BrettPhos Pd G3 (0.1 eq) and Cs₂CO₃ (3.00 eq) in DMF was stirred at 100° C. for 4 hours under nitrogen atmosphere. The mixture was treated with metal scavenger, then filtered, the filtrate was purified by Prep-HPLC to afford (2R,3R,4R,5S)-5-((6-(trifluoromethoxy)pyridin-2-yl)amino)-2-(((6-(trifluoromethoxy)pyridin-2-yl)oxy)methyl)tetrahydro-2H-pyran-3,4-diol (A125) as a light yellow solid. Yield: 5%. Chemical Formula: C₁₈H₁₇F₆N₃O₆. LCMS found: [M+H]⁺=486.1. ¹H NMR (400 MHz, DMSO) δ 7.92 (t, J=8.0 Hz, 1H), 7.51 (t, J=8.0 Hz, 1H), 6.89-6.84 (m, 3H), 6.49 (d, J=8.0 Hz, 1H), 6.23 (d, J=8.0 Hz, 1H), 4.86 (d, J=4.7 Hz, 1H), 4.80 (d, J=6.6 Hz, 1H), 4.40-4.25 (m, 2H), 4.05-3.87 (m, 2H), 3.84-3.80 (m, 1H), 3.74-3.68 (m, 1H), 3.57-3.51 (m, 1H), 3.00-2.92 (m, 1H).

### Preparation of (2R,3R,4R,5S)-5-((6-chloropyridin-2-yl)amino)-2-(hydroxymethyl) tetrahydro-2H-pyran-3,4-diol (A145) and (2R,3R,4R,5S)-5-((6-chloropyridin-2-yl)amino)-2-(((6-chloropyridin-2-yl)oxy)methyl) tetrahydro-2H-pyran-3,4-diol (A146)

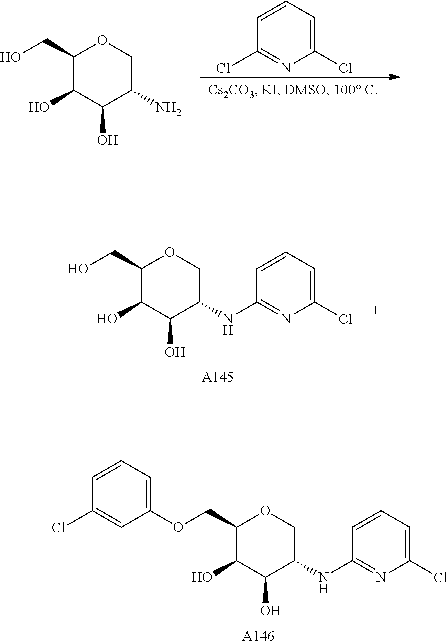

To a solution of (2R,3R,4R,5S)-5-amino-2-(hydroxymethyl)tetrahydro-2H-pyran-3,4-diol (1.00 eq) in DMSO (4 mL) was added 2,6-dichloropyridine (1.10 eq), Cs₂CO₃ (1.50 eq) and KI (0.20 eq). The mixture was stirred at 100° C. for 2 h under N₂ atmosphere. Both of the two desired was detected by LCMS. The mixture was filtered, the filtrate was purified by prep-HPLC to afford (2R,3R,4R,5S)-5-((6-chloropyridin-2-yl)amino)-2-(hydroxymethyl)tetrahydro-2H-pyran-3,4-diol (A127) as a white solid. Yield: 10%. Chemical Formula: C₁₁H₁₅ClN₂O₄, LCMS found: [M+H]⁺=275. ¹H NMR (400 MHz, CD₃OD) δ 7.34 (t, J=7.9 Hz, 1H), 6.51 (d, J=7.6 Hz, 1H), 6.45 (d, J=8.4 Hz, 1H), 4.21 (td, J=10.5, 5.2 Hz, 1H), 4.11 (dd, J=11.0, 5.2 Hz, 1H), 3.89 (d, J=3.0 Hz, 1H), 3.75 (dd, J=11.2, 7.2 Hz, 1H), 3.68 (dd, J=11.2, 5.2 Hz, 1H), 3.56 (dd, J=10.4, 3.2 Hz, 1H), 3.47-3.39 (m, 1H), 3.09 (t, J=10.8 Hz, 1H).

The mixture was purified by prep-HPLC to afford (2R,3R,4R,5S)-5-((6-chloropyridin-2-yl)amino)-2-(((6-chloropyridin-2-yl)oxy)methyl)tetrahydro-2H-pyran-3,4-diol (A126) as a brown solid. Yield: 7%. Chemical Formula: C₁₆H₁₇C₁₂N₃O₄, LCMS found: [M+H]⁺=386. ¹H NMR (400 MHz, DMSO-d₆) δ 7.73 (t, J=8.0 Hz, 1H), 7.36 (t, J=8.0 Hz, 1H), 7.03 (d, J=8.0 Hz, 1H), 6.90 (d, J=8.0 Hz, 1H), 6.84 (d, J=7.6 Hz, 1H), 6.49 (d, J=7.2 Hz, 1H), 6.46 (d, J=8.0 Hz, 1H) 5.56 (d, J=3.2 Hz, 1H), 5.03 (d, J=6.2 Hz, 1H), 4.62 (t, J=5.6 Hz, 1H), 4.13-3.94 (m, 2H) 3.85-3.70 (m, 1H), 3.60 (t, J=6.2 Hz, 1H), 3.41-3.38 (m, 2H), 3.00 (t, J=10.6 Hz, 1H).

### Preparation of (2R,3R,4R,5S)-5-((6-chloropyrazin-2-yl)amino)-2-(hydroxymethyl) tetrahydro-2H-pyran-3,4-diol (A147)

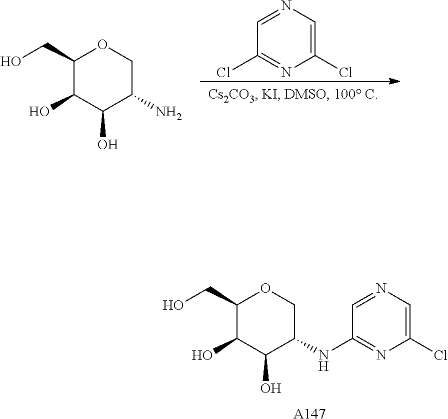

To a solution of (2R,3R,4R,5S)-5-amino-2-(hydroxymethyl)tetrahydro-2H-pyran-3,4-diol (1.00 eq) in DMSO (4 mL) was added 2,6-dichloropyrazine (1.10 eq), Cs₂CO₃ (1.50 eq) and KI (0.20 eq). The mixture was stirred at 100° C. for 2 h under N₂ atmosphere. The mixture was filtered, the filtrate was purified by prep-HPLC to afford (2R,3R,4R,5S)-5-((6-chloropyrazin-2-yl)amino)-2-(hydroxymethyl)tetrahydro-2H-pyran-3,4-diol (A128) as a brown solid. Yield: 25%. Chemical Formula: C₁₀H₁₄ClN₃O₄, LCMS found: [M+H]⁺=276. ¹H NMR (400 MHz, CD₃OD) δ 7.71 (s, 1H), 7.54 (s, 1H), 4.21 (td, J=10.4, 5.2 Hz, 1H), 4.00 (dd, J=10.8, 5.2 Hz, 1H), 3.82 (d, J=3.2 Hz, 1H), 3.77 (dd, J=11.2, 7.2 Hz, 1H), 3.69 (dd, J=11.2, 5.2 Hz, 1H), 3.61 (dd, J=10.4, 3.2 Hz, 1H), 3.52-3.45 (m, 1H), 3.39-3.31 (m, 1H), 3.02 (t, J=10.8 Hz, 1H).

### Preparation of (4-benzylphenyl)((3S,4R,5S)-3,4-dihydroxy-5-((6-(trifluoromethyl) pyrazin-2-yl) amino)piperidin-1-yl)methanone (A148)

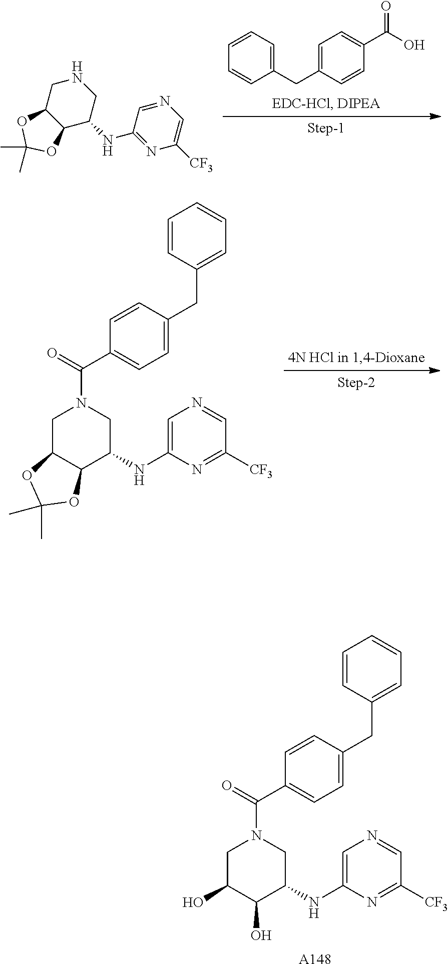

Step 1: To a solution of (3aS,7S,7aR)-2,2-dimethyl-N-(6-(trifluoromethyl)pyrazin-2-yl)hexahydro-[1,3]dioxolo[4,5-c]pyridin-7-amine (30 mg, 0.094 mmol) in DMF were added 4-benzylbenzoic acid (30 mg, 0.141 mmol), EDCl·HCl (20 mg, 0.141 mmol) followed by DIPEA (0.05 mL, 0.282 mmol) at 0° C. The reaction mixture was stirred at rt for 16 h. reaction mixture was diluted with EtOAc (50 mL) and washed with H₂O (2×10 mL). The organic layer was dried over Na₂SO₄, filtered, concentrated under reduced pressure to give (4-benzylphenyl)((3aS,7S,7aR)-2,2-dimethyl-7-((6-(trifluoromethyl)pyrazin-2-yl)amino)tetrahydro-[1,3]dioxolo[4,5-c]pyridin-5(4H)-yl)methanone (40 mg, crude). This was used for next step without purification. LC-MS (ESI) found: 513.30 [M+H]⁺. (83% of desired mass).
Step 2: To a solution of (4-benzylphenyl)((3aS,7S,7aR)-2,2-dimethyl-7-((6-(trifluoromethyl)pyrazin-2-yl)amino)tetrahydro-[1,3]dioxolo[4,5-c]pyridin-5(4H)-yl)methanone (40 mg, crude) in DCM (4.0 mL) was added 4N HCl in 1,4-Dioxane (0.5 mL) at 0° C. The reaction mixture was stirred at rt for 1 h. After completion of the starting material, all the volatiles were removed under reduced pressure to give crude product which was purified by prep-HPLC to give (4-benzylphenyl)((3S,4R,5S)-3,4-dihydroxy-5-((6-(trifluoromethyl)pyrazin-2-yl)amino)piperidin-1-yl)methanone (A129) (10 mg, 27% yield). 1H NMR (400 MHz, DMSO-d₆): δ 8.20-8.29 (m, 1H), 7.93-8.15 (m, 1H), 7.67 (s, 1H), 7.35-7.46 (m, 1H), 7.18-7.32 (m, 5H), 7.07-7.16 (m, 2H), 6.96 (d, J=6.24 Hz, 1H), 4.88-5.16 (m, 2H), 3.65-4.11 (m, 5H), 3.39-3.54 (m, 2H), 3.07-3.25 (m, 1H), 2.82-2.98 (m, 1H). LC-MS (ESI) found: 473.25 [M+H]⁺.

### Preparation of benzyl (3S,4R,5S)-3,4-dihydroxy-5-((6-(trifluoromethyl)pyrazin-2-yl)amino)piperidine-1-carboxylate (A149)

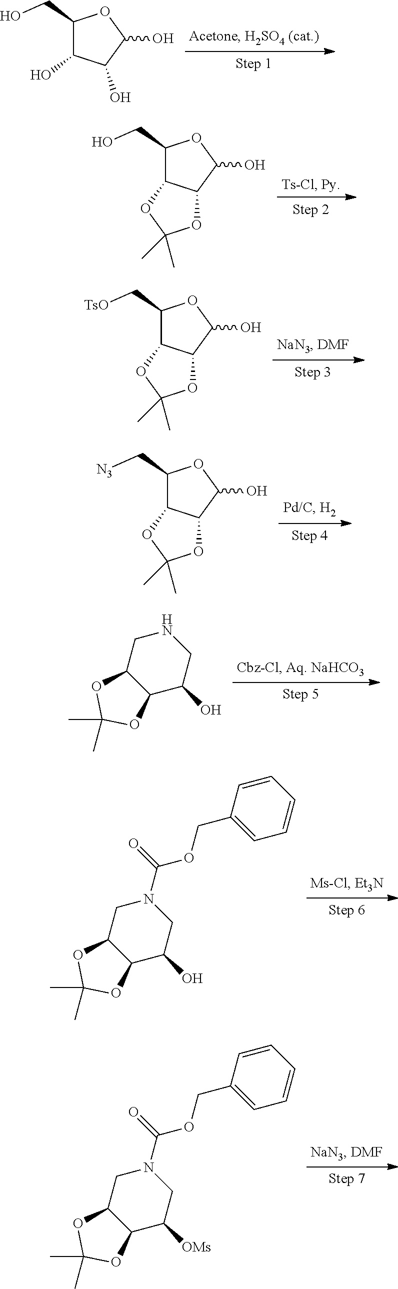

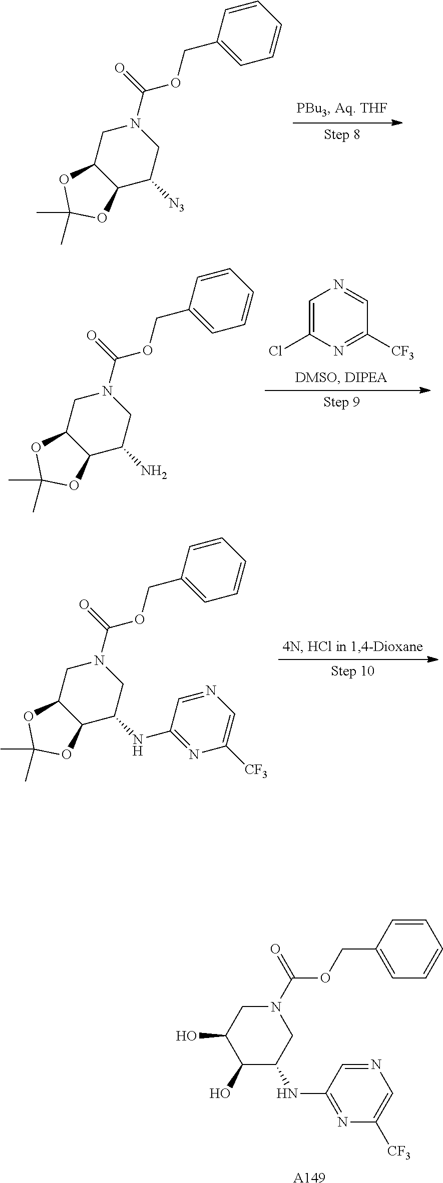

Step-1: To a solution of D-Ribose (100 g, 666.66 mmol) in acetone (1 L) was added H₂SO₄ (10 mL) at 0° C. Reaction mixture was stirred at rt for 16 h. The mixture was neutralised with solid NaHCO₃ at rt. The solid was filtered, and the filtrate was concentrated under reduced pressure. The crude was purified by column chromatography using EtOAc in hexane (8:2) as an eluants to give (3aR,6R,6aR)-6-(hydroxymethyl)-2,2-dimethyltetrahydrofuro[3,4-d][1,3]dioxol-4-ol (70 g, 55% yield) as a colourless liquid. ¹H NMR (400 MHz, DMSO-d₆) δ 6.42-6.49 (s, 1H), 5.15 (s, 1H) 4.91 (s, 1H), 4.68 (d, J=5.87 Hz, 1H), 4.40-4.48 (m, 1H), 3.94-4.05 (m, 1H), 3.42 (s, 2H) 1.36 (s, 3H), 1.23-1.26 (s, 3H).
Step-2: To a solution of (3aR,6R,6aR)-6-(hydroxymethyl)-2,2-dimethyltetrahydrofuro[3,4-d][1,3]dioxol-4-ol (70 g, 368.42 mmol) in pyridine (700 mL) was added tosyl chloride (104 g, 552.63 mmol) in portion wise at 0° C. for 2 h. The mixture was stirred at 25° C. for 4 h. The mixture was diluted with EtOAc (500 mL) and washed with water (200 mL×3). The organic layer was dried over Na₂SO₄, filtered, concentrated under reduced pressure to give ((3aR,4R,6aR)-6-hydroxy-2,2-dimethyltetrahydrofuro[3,4-d][1,3]dioxol-4-yl)methyl 4-methylbenzenesulfonate (80 g, crude). This crude product was used for next step without purification. 1H NMR (400 MHz, DMSO-d₆) δ 8.56 (s, 1H), 7.75-7.77 (d, 2H), 7.49 (s, 2H), 6.71 (d, 1H), 5.20 (d, 2H), 4.42-4.42 (m, 1H), 4.07 (s, 2H), 4.04 (s, 1H), 2.41-2.42 (m, 3H), 1.36 (s, 3H), 1.22 (s, 3H).
Step-3: To a solution of ((3aR,4R,6aR)-6-hydroxy-2,2-dimethyltetrahydrofuro[3,4-d][1,3]dioxol-4-yl)methyl 4-methylbenzenesulfonate (80 g, crude) in DMF (800 mL) was added NaN₃ (22 g, 338 mmol) at rt. Reaction mixture was heated at 100° C. for 16 h. The mixture was diluted with EtOAc (200 mL) and washed with H₂O (200 mL×3). The organic layer was dried over Na₂SO₄, filtered, concentrated under reduced pressure to give crude product, which was purified by column chromatography to give (3aR,6R,6aR)-6-(azidomethyl)-2,2-dimethyltetrahydrofuro[3,4-d][1,3]dioxol-4-ol (40 g, 55% yield). 1H NMR (400 MHz, DMSO-d₆) δ 6.77-6.75 (d, J=4.4 Hz, 1H), 5.25 (d, J=3.91 Hz, 1H), 4.63 (d, J=5.87 Hz, 1H), 4.51 (d, J=5.87 Hz, 1H), 4.04-4.11 (m, 1H), 3.47-3.56 (m, 1H), 3.34 (m, 1H), 1.38 (s, 3H), 1.25 (s, 3H).
Step-4: To a solution of (3aR,6R,6aR)-6-(azidomethyl)-2,2-dimethyltetrahydrofuro[3,4-d][1,3]dioxol-4-ol (40 g, 186 mmol) in EtOH (1.6 L) was added 10% Pd/C (10 g) at rt. Reaction mixture was stirred under H2 atmosphere in auto clave at 60 PSI for 16 h at rt. The reaction mixture was filtered through a celite pad and the solvent was concentrated to give crude product. Crude was washed with diethyl ether (200 mL) to give (3aS,7R,7aR)-2,2-dimethylhexahydro-[1,3]dioxolo[4,5-c]pyridin-7-ol (20 g, 62% yield) as an off white solid. ¹H NMR (400 MHz, DMSO-d₆) δ 4.76 (d, J=6.25 Hz, 1H), 4.20 (t, J=4.38 Hz, 1H), 3.90-3.99 (m, 1H), 3.56-3.63 (m, 1H), 2.76 (dd, J=12.51, 6.38 Hz, 1H), 2.61-2.65 (m, 1H), 2.40-2.44 (m, 1H), 2.37 (d, J=7.75 Hz, 1H), 2.29-2.34 (s, 1H), 1.42 (s, 3H), 1.26 (s, 3H).
Step-5: To a solution of (3aS,7R,7aR)-2,2-dimethylhexahydro-[1,3]dioxolo[4,5-c]pyridin-7-ol (30 g, 173 mmol) in THF (300 mL) were added aq. saturated NaHCO₃ solution (150 mL) followed benzyl chloroformate (35 g, 207.6 mmol) at 0° C. Reaction mixture was stirred at rt for 16 h. Reaction mixture was diluted with EtOAc (1 L) and washed with H₂O (2×200 mL). The organic layer was dried over Na₂SO₄, filtered, concentrated under reduced pressure to give a crude product, which was purified by column chromatography using to give Benzyl (3aS,7R,7aR)-7-hydroxy-2,2-dimethyltetrahydro-[1,3]dioxolo[4,5-c]pyridine-5(4H)-carboxylate (35 g, 66% yield) as colour less liquid. ¹H NMR (400 MHz, DMSO-d₆) δ 7.25-7.38 (m, 5H), 5.01-5.18 (m, 3H) 4.31 (s, 2H), 3.83 (dd, J=10.76, 5.38 Hz, 1H), 3.62 (d, J=14.18 Hz, 1H), 3.05-3.25 (m, 2H) 1.25-1.31 (m, 6H).
Step-6: To a solution of benzyl (3aS,7R,7aR)-7-hydroxy-2,2-dimethyltetrahydro-[1,3]dioxolo[4,5-c]pyridine-5(4H)-carboxylate (35 g, 114 mmol) in DCM (350 mL) were added Et₃N (48 mL, 342 mmol) and mesyl chloride (20 g, 171 mmol) at 0° C. DMAP (2.7 g, 22.8 mmol) was also added. The reaction mixture was stirred at rt for 4 h. Reaction was diluted with DCM (500 mL) and washed with H₂O (2×200 mL). The organic layer was dried over Na₂SO₄, filtered, concentrated under reduced pressure to give a crude product, which was purified by column chromatography, (PE/EA=7/3) to give benzyl (3aS,7R,7aS)-2,2-dimethyl-7-((methylsulfonyl)oxy)tetrahydro-[1,3]dioxolo[4,5-c]pyridine-5(4H)-carboxylate (32 g, 74% yield) as colourless liquid. ¹H NMR (400 MHz, DMSO-d₆) δ 7.31-7.36 (m, 5H), 5.00-5.12 (m, 3H), 4.35-4.53 (m, 2H), 3.71-3.78 (m, 1H), 3.43-3.55 (m, 2H), 3.35 (s, 1H), 3.31 (s, 3H) 1.29-1.36 (m, 6H).
Step-7: To a solution of benzyl (3aS,7R,7aS)-2,2-dimethyl-7-((methylsulfonyl)oxy)tetrahydro-[1,3]dioxolo[4,5-c]pyridine-5(4H)-carboxylate (8 g, 20 mmol) in DMF (80 mL, 10 vol) was added NaN₃ (5.4 g, 80 mmol) at rt. The reaction mixture was heated at 120° C. for 36 h. After completion of the reaction, cooled to rt. Reaction mixture was diluted with EtOAc (500 mL) and washed with H₂O (2×100 mL). The organic layer was dried over Na₂SO₄, filtered, concentrated under reduced pressure to give a crude product, which was purified by column chromatography, (PE/EA=7/3) to give benzyl(3aS,7S,7aR)-7-azido-2,2-dimethyltetrahydro-[1,3]dioxolo[4,5-c]pyridine-5(4H)-carboxylate (2.2 g, 32% yield). ¹H NMR (400 MHz, CDCl₃) δ 7.25-7.40 (m, 5H), 5.17 (s, 2H) 4.25-4.35 (m, 1H), 4.03-4.16 (m, 2H), 3.64-3.90 (m, 2H), 3.16-3.48 (m, 2H), 1.47 (s, 3H) 1.35 (s, 3H).
Step-8: To a solution benzyl (3aS,7S,7aR)-7-azido-2,2-dimethyltetrahydro-[1,3]dioxolo[4,5-c]pyridine-5(4H)-carboxylate (2 g, 6.02 mmol) in THF (20 mL) was added n-tributyl phosphine (2.4 g, 12.04 mmol) at 0° C. The reaction mixture was stirred at rt for 2 h. After completion of starting material by TLC, water (10 mL) was added, and the reaction mixture was heated at 70° C. for 1 h. The reaction mixture was cooled to rt, diluted with EtOAc (200 mL) and washed with H₂O (2×50 mL). The organic layer was dried over Na₂SO₄, filtered, concentrated under reduced pressure to give a crude product, which was purified by column chromatography, (DCM/MeOH=9/1) to give benzyl(3aS,7S,7aR)-7-amino-2,2-dimethyltetrahydro-[1,3]dioxolo[4,5-c]pyridine-5(4H)-carboxylate (800 mg, 44% yield). ¹H NMR (400 MHz, DMSO-d₆) δ 7.20-7.52 (m, 4H) 4.88-5.25 (m, 2H), 4.25 (s, 1H), 3.68-3.92 (m, 2H), 3.35-3.60 (m, 2H), 2.79-3.02 (m, 2H) 1.53-1.73 (m, 2H), 1.31-1.40 (m, 3H), 1.26 (s, 3H). LC-MS (ESI) found: 307 [M+H]⁺.
Step-9: To a solution of benzyl(3aS,7S,7aR)-7-amino-2,2-dimethyltetrahydro-[1,3]dioxolo[4,5-c]pyridine-5(4H)-carboxylate (500 mg, 1.62 mmol) in DMSO (5 mL) were added 2-chloro-6-(trifluoromethyl)pyrazine) (600 mg, 3.3 mmol), followed by DIPEA (1.0 mL, 4.84 mmol) at rt. The reaction mixture was irradiated under microwave at 120° C. for 4 h. After completion of the reaction, cooled to rt, diluted with EtOAc (100 mL) and washed with H₂O (2×50 mL). The organic layer was dried over Na₂SO₄, filtered, concentrated under reduced pressure to give a crude product, which was purified by column chromatography, (DCM/MeOH=9/1) to give benzyl(3aS,7S,7aR) 2,2-dimethyl-7-((6-(trifluoromethyl)pyrazin-2-yl)amino)tetrahydro-[1,3]dioxolo[4,5-c]pyridine-5(4H)-carboxylate (450 mg, 61% yield). ¹H NMR (400 MHz, DMSO-d₆) δ 8.24 (s, 1H), 8.14 (s, 1H), 7.92 (s, 1H), 7.22-7.45 (m, 5H), 5.04-5.19 (m, 2H), 4.35 (s, 1H), 3.95-4.18 (m, 3H) 3.75 (d, J=10.27 Hz, 1H), 3.44 (d, J=11.25 Hz, 1H), 2.55-2.71 (m, 1H), 1.39 (s, 3H), 1.27 (s, 3H). LC-MS (ESI) found: 453.59 [M+H]⁺.
Step-10: To a solution of benzyl(3aS,7S,7aR)-2,2-dimethyl-7-((6-(trifluoromethyl)pyrazin-2-yl)amino)tetrahydro-[1,3]dioxolo[4,5-c]pyridine-5(4H)-carboxylate (50 mg, 0.11 mmol) in DCM (4.0 mL) was added 4N HCl in 1,4-Dioxane (0.5 mL) at 0° C. The reaction mixture was stirred at rt for 1 h. After completion of the starting material, concentrated under reduced pressure to give crude product which was purified through prep-HPLC to give benzyl (3S,4R,5S)-3,4-dihydroxy-5-((6-(trifluoromethyl)pyrazin-2-yl)amino)piperidine-1-carboxylate (A130) (10 mg, 22% yield). ¹H NMR (400 MHz, DMSO-d₆) δ 8.24 (s, 1H), 8.08 (s, 1H), 7.66 (s, 1H), 7.25-7.37 (m, 5H) 4.90-5.13 (m, 4H), 3.73-4.07 (m, 4H), 3.40-3.70 (m, 3H). LC-MS (ESI) found: 413.2 [M+H]⁺.
A149: ¹H NMR (400 MHz, DMSO-d₆) δ ppm 8.52 (s, 1H) 8.12 (s, 1H) 4.67-4.73 (m, 1H) 4.63 (d, J=3.88 Hz, 1H) 4.21-4.34 (m, 1H) 3.74 (d, J=2.00 Hz, 2H) 2.97 (s, 3H) 2.79 (d, J=13.13 Hz, 2H) 2.53-2.59 (m, 3H) LC-MS (ESI) found: 293.0 [M+H]⁺.

### Preparation of (3S,4R,5S)-5-((6-(trifluoromethyl) pyrazin-2-yl)amino)piperidine-3,4-diol (A150)

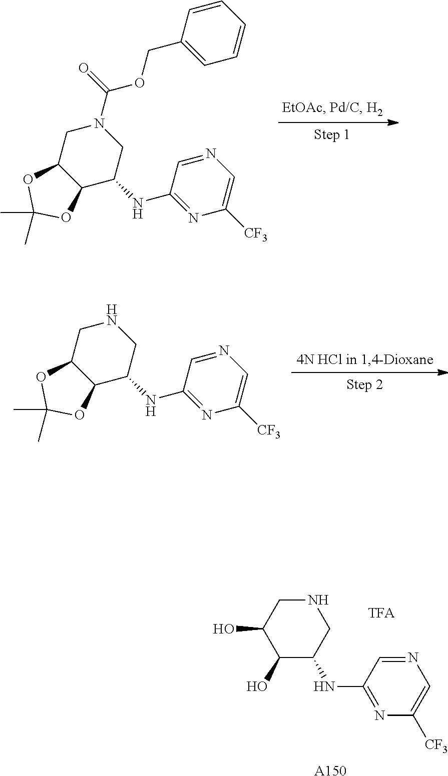 [SMILES: CC1(C)O[C@@H]2[C@@H](Nc3cncc(C(F)(F)F)n3)CN(C(=O)OCc3ccccc3)C[C@@H]2O1 ; CC1(C)O[C@@H]2[C@@H](Nc3cncc(C(F)(F)F)n3)CNC[C@@H]2O1 ; O[C@@H]1[C@@H](Nc2cncc(C(F)(F)F)n2)CNC[C@@H]1O ; "A150"]

Step-1: To a solution of benzyl(3aS,7S,7aR)-2,2-dimethyl-7-((6-(trifluoromethyl)pyrazin-2-yl)amino)tetrahydro-[1,3]dioxolo[4,5-c]pyridine-5(4H)-carboxylate (50 mg, 0.11 mmol) in EtOAc (3.0 mL) was added 10% Pd/C (10 mg) at rt. The reaction mixture was stirred under H2 atmosphere (balloon pressure) at rt for 16 h. The mixture was filtered through a celite pad, and the organic layer was concentrated to give (3aS,7S,7aR)-2,2-dimethyl-N-(6-(trifluoromethyl)pyrazin-2-yl)hexahydro-[1,3]dioxolo[4,5-c]pyridin-7-amine (30 mg, crude). This was used for next step without purification. ¹H NMR (400 MHz, DMSO-d₆) δ 8.2 (s, 1H), 8.05 (s, 1H), 7.70-7.68 (d, J=8.0 Hz, 1H), 4.09-4.08 (d, J=4.0 Hz, 1H), 3.95-3.92 (m, 2H), 3.31-2.86 (m, 1H), 2.85-2.81 (m, 2H), 2.32-2.21 (m, 1H), 1.4 (s, 3H), 1.25 (s, 3H). LC-MS (ESI) found: 319.3 [M+H]⁺. (50% of desire mass).
Step-2: To a solution of (3aS,7S,7aR)-2,2-dimethyl-N-(6-(trifluoromethyl)pyrazin-2-yl)hexahydro-[1,3]dioxolo[4,5-c]pyridin-7-amine (30 mg, crude) in DCM (4.0 mL) was added 4N-HCl in 1,4-Dioxane (0.5 mL) at 0° C. The reaction mixture was stirred at rt for 1 hr. After completion of the starting material, volatiles were removed under reduced pressure to give crude product which was purified by prep-HPLC to give (3S,4R,5S)-5-((6-(trifluoromethyl)pyrazin-2-yl)amino)piperidine-3,4-diol as TFA salt (A131) (10 mg, 38% yield). ¹H NMR (400 MHz, DMSO-d₆) δ 8.66-8.96 (m, 1H), 8.21-8.31 (m, 1H), 8.09-8.20 (m, 1H), 7.81 (d, J=7.40 Hz, 1H) 5.72 (s, 1H), 5.28 (s, 1H), 4.29 (d, J=4.16 Hz, 1H), 4.02 (s, 1H), 3.65 (d, J=8.79 Hz, 1H), 3.28 (s, 1H), 3.14 (s, 2H), 2.69 (d, J=15.26 Hz, 1H). LC-MS (ESI) found: 279.3 [M+H]⁺.
A150: ¹H NMR (400 MHz, DMSO-d₆) δ ppm 8.47 (s, 1H) 8.15 (s, 1H) 4.67 (d, J=5.50 Hz, 1H) 4.48-4.60 (m, 1H) 4.41 (d, J=5.38 Hz, 1H) 3.80 (d, J=3.06 Hz, 1H) 3.61-3.70 (m, 1H) 3.00 (s, 3H) 2.82-2.88 (m, 1H) 2.74-2.81 (m, 1H) 2.32-2.40 (m, 2H) 2.06-2.14 (m, 2H) 0.98 (t, J=7.15 Hz, 3H) LC-MS (ESI) found: 321.2 [M+H]⁺.

### Preparation of (2R,3R,4R,5S)-2-(hydroxymethyl)-5-((4-methoxy-6-(trifluoromethyl)pyridin-2-yl)amino)tetrahydro-2H-pyran-3,4-diol (A151)

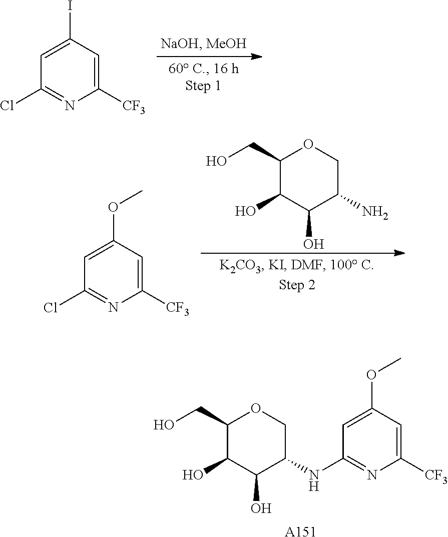

Step 1: To a solution of 2-chloro-4-iodo-6-(trifluoromethyl)pyridine (0.5 g, 1.00 eq) in MeOH (10 mL) was added NaOH (0.2 g, 3.00 eq), the mixture was stirred at 60° C. for 16 hours. The reaction was quenched with water and then extracted with DCM, the organic layers was dried over Na₂SO₄, filtered and concentrated reduced pressure, the residue was purified by silica gel chromatography (PE/EA=50/1 to 3/1) to afford 2-chloro-4-methoxy-6-(trifluoromethyl)pyridine (0.25 g) as a light yellow solid. Yield: 72.7%. Chemical Formula: C₇H₅ClF₃NO, LCMS found: [M+H]⁺=212.
Step 2: To a solution of 2-chloro-4-methoxy-6-(trifluoromethyl)pyridine (50 mg, 1.00 eq) and (2R,3R,4R,5S)-5-amino-2-(hydroxymethyl)tetrahydro-2H-pyran-3,4-diol (38 mg, 1.00 eq) in DMF (5 mL) was added K₂CO₃ (164 mg, 3.00 eq) and KI (5 mg, 0.1 eq), the mixture was stirred at 100° C. for 16 h. The reaction was filtered, the filtrate was concentrated under reduced pressure. The residue was purified by reverse phase (C18, 5-40% MeCN in water, 0.1% TFA) to afford (2R,3R,4R,5S)-2-(hydroxymethyl)-5-((4-methoxy-6-(trifluoromethyl)pyridin-2-yl)amino)tetrahydro-2H-pyran-3,4-diol (A132) (3 mg) as white solid. Yield: 3.7%. Chemical Formula: C₁₃H₁₇F₃N₂O₅, LCMS found: [M+H]⁺=339. ¹H NMR (400 MHz, CD₃OD) δ 6.51 (d, J=1.6 Hz, 1H), 6.21 (d, J=1.6 Hz, 1H), 4.29-4.12 (m, 2H), 3.89 (d, J=3.3 Hz, 1H), 3.82 (s, 3H) 3.75 (dd, J=11.2, 7.2 Hz, 1H), 3.68 (dd, J=11.2, 5.2 Hz, 1H), 3.58 (dd, J=10.4, 3.2 Hz, 1H) 3.47-3.42 (m, 1H), 3.11 (t, J=10.8 Hz, 1H).

### Preparation of 2-(((2R,3R,4R,5S)-3,4-dihydroxy-5-((6-(trifluoromethyl)pyrazin-2-yl)amino) tetrahydro-2H-pyran-2-yl)methyl) isoindolin-1-one (A152)

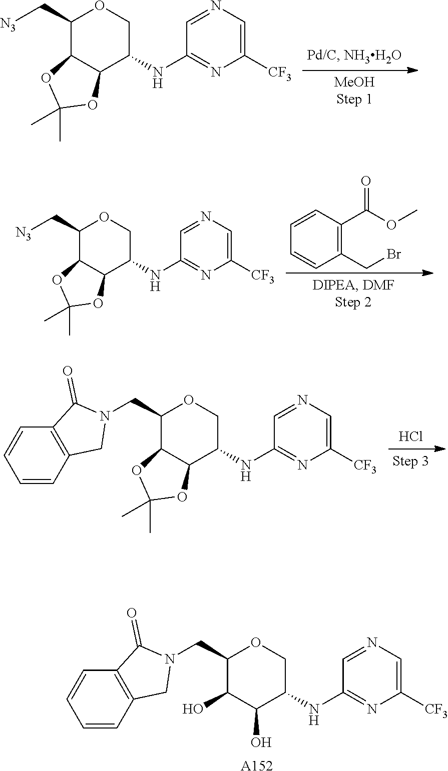

Step 1: To a solution of N-((3aS,4R,7S,7aR)-4-(azidomethyl)-2,2-dimethyltetrahydro-4H-[1,3]dioxolo[4,5-c]pyran-7-yl)-6-(trifluoromethyl)pyrazin-2-amine (1.00 eq) in MeOH (9.0 mL) was added Pd/C (10% purity) and NH₃·H₂O (1.00 eq). Then the mixture was stirred at 25° C. for 1 h under hydrogen atmosphere (15 psi). The mixture was filtered, the filtrate was concentrated under reduced pressure to give N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (crude) as a brown solid. Yield: 90%. Chemical Formula: C₁₄H₁₉F₃N₄O₃, LCMS found: [M+H]⁺=349.2.
Step 2: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.00 eq) in DMF (4.0 mL) was added methyl 2-(bromomethyl)benzoate (1.60 eq) and DIPEA (2.00 eq). The mixture was stirred at 100° C. for 12 hours. The mixture was concentrated under reduced pressure. The residue was purified by silica gel chromatography (DCM/MeOH=50/1 to 15/1) to give 2-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)isoindolin-1-one as brown oil. Yield: 60%. Chemical Formula: C₂₂H₂₃F₃N₄O₄, LCMS found: [M+H]⁺=465.2.
Step 3: To a solution of 2-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)isoindolin-1-one (1.00 eq) in THE (10 mL) and H₂O (2 mL) was added 2N HCl (10.00 eq). The mixture was stirred at 25° C. for 12 hours. The mixture was concentrated under reduced pressure. The residue was purified by reverse phase (C18, 5-21% MeCN in water, 0.1% FA) to afford 2-(((2R,3R,4R,5S)-3,4-dihydroxy-5-((6-(trifluoromethyl)pyrazin-2-yl)amino)tetrahydro-2H-pyran-2-yl)methyl)isoindolin-1-one (A133) as white solid. Yield: 65%. Chemical Formula: C₁₉H₁₉F₃N₄O₄, LCMS found: [M+H]⁺=425.3. ¹H NMR (400 MHz, CD₃OD) δ 8.10 (s, 1H), 7.99 (s, 1H), 7.77 (d, J=7.6 Hz, 1H), 7.62-7.54 (m, 2H), 7.49 (t, J=7.2 Hz, 1H), 4.72 (d, J=18.0 Hz, 1H), 4.55 (d, J=18.0 Hz, 1H), 4.36 (td, J=10.6, 5.2 Hz, 1H), 4.13 (dd, J=11.2, 5.2 Hz, 1H), 3.97 (d, J=10.4 Hz, 1H), 3.90 (d, J=3.2 Hz, 1H), 3.82-3.72 (m, 2H), 3.67 (dd, J=10.6, 3.2 Hz, 1H), 3.08 (t, J=10.8 Hz, 1H).

### Preparation of (2R,3R,4R,5S)-5-((6-bromopyridin-2-yl) amino)-2-(hydroxymethyl) tetrahydro-2H-pyran-3,4-diol (A153)

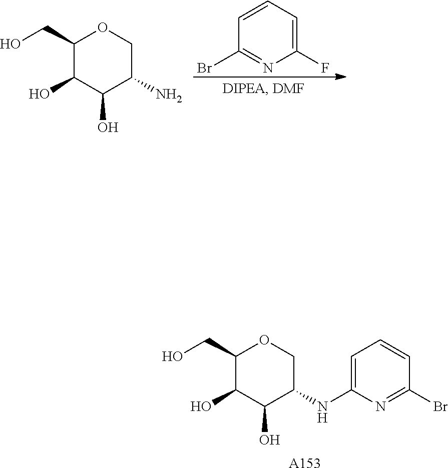

To a solution of (2R,3R,4R,5S)-5-amino-2-(hydroxymethyl)tetrahydro-2H-pyran-3,4-diol (1.00 eq) in DMF (4 mL) was added 2-bromo-6-fluoropyridine (1.60 eq) and DIPEA (3.00 eq). The mixture was stirred at 100° C. for 12 hours. The mixture was concentrated under reduced pressure. The residue was purified by reverse phase (C18, 5-15% acetonitrile in water, 0.1% FA) to afford (2R,3R,4R,5S)-5-((6-bromopyridin-2-yl)amino)-2-(hydroxymethyl)tetrahydro-2H-pyran-3,4-diol (A134) as a brown solid. Yield: 15%. Chemical Formula: C₁₁H₁₅BrN₂O₄, LCMS found: [M+H]+ 5=319.1, 321.1. ¹H NMR (400 MHz, D₂O): δ 7.40 (t, J=8.0 Hz, 1H), 6.86 (d, J=8.0 Hz, 1H), 6.61 (d, J=8.0 Hz, 1H), 4.13-4.05 (m, 2H), 4.00 (d, J=3.2 Hz, 1H), 3.82-3.69 (m, 3H), 3.62 (dd, J=7.6, 4.3 Hz, 1H), 3.28-3.16 (m, 1H).

### Preparation of(2R,3R,4R,5S)-2-(((6-fluoropyrazin-2-yl)oxy)methyl)-5-((5-fluoropyridin-3-yl)amino)tetrahydro-2H-pyran-3,4-diol (A154)

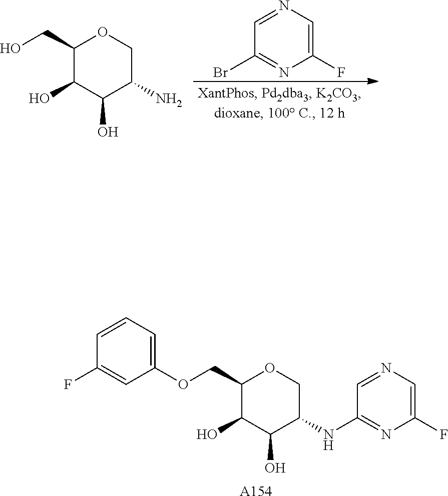

To a mixture of (2R,3R,4R,5S)-5-amino-2-(hydroxymethyl)tetrahydro-2H-pyran-3,4-diol (1.00 eq), 2-bromo-6-fluoropyrazine (1.00 eq) in dioxane was added XantPhos (0.2 eq), Pd₂(dba)₃ (0.05 eq) and K₂CO₃ (2.00 eq), the mixture was stirred at 100° C. for 12 hours under nitrogen atmosphere. The mixture was treated with metal scavenger, then filtered, the filtrate was concentrated under reduced pressure, the residue was purified by Prep-HPLC to afford (2R,3R,4R,5S)-5-((6-fluoropyrazin-2-yl)amino)-2-(((6-fluoropyrazin-2-yl)oxy)methyl)tetrahydro-2H-pyran-3,4-diol (A135) as a yellow solid. Chemical Formula: C₁₄H₁₅F₂N₅O₄. LCMS found: [M+H]⁺=356.3. ¹H NMR (400 MHz, DMSO-d₆) δ 8.35 (d, J=4.3 Hz, 1H), 8.23 (d, J=7.9 Hz, 1H), 7.88 (d, J=5.3 Hz, 1H), 7.53 (d, J=8.4 Hz, 1H), 7.48 (d, J=7.9 Hz, 1H), 4.98 (d, J=4.9 Hz, 1H), 4.89 (d, J=6.5 Hz, 1H), 4.42-4.36 (m, 2H), 4.09-4.00 (m, 1H), 3.89 (dd, J=10.9, 4.9 Hz, 1H), 3.86-3.83 (m, 1H), 3.81-3.76 (m, 1H), 3.58-3.52 (m, 1H), 3.03 (t, J=10.8 Hz, 1H).

### Preparation of (2R,3R,4R,5S)-2-methyl-5-((6-(trifluoromethyl)pyrazin-2-yl)amino) tetrahydro-2H-pyran-3,4-diol (A155)

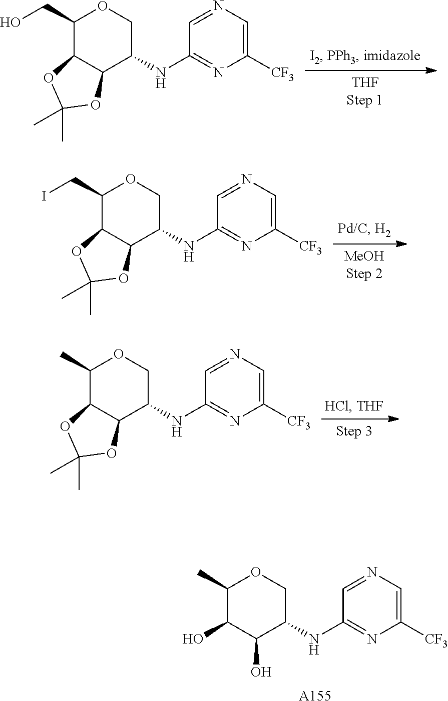

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methanol (1.00 eq), PPh₃ (2.00 eq) and Imidazole (2.00 eq) in THE was added 12 (1.50 eq). The mixture was stirred at 65° C. for 5 hours, then concentrated. The residue was purified by silica gel chromatography (PE/EA=20/1 to 3/1) to afford N-((3aR,4S,7S,7aR)-4-(iodomethyl)-2,2-dimethyltetrahydro-4H-[1,3]dioxolo[4,5-c]pyran-7-yl)-6-(trifluoromethyl)pyrazin-2-amine as colorless oil. Yield: 80%. LCMS found: [M+H]⁺=460.
Step 2: A solution of N-((3aR,4S,7S,7aR)-4-(iodomethyl)-2,2-dimethyltetrahydro-4H-[1,3]dioxolo[4,5-c]pyran-7-yl)-6-(trifluoromethyl)pyrazin-2-amine (1.00 eq) and Pd/C in MeOH (3 mL) was stirred at 20° C. under H2 for 16 hours. The mixture was filtered through a Celite pad, and the filtrate was concentrated to give crude product. The residue was purified by silica gel chromatography (PE/EA=10/1 to 1/1) to afford 6-(trifluoromethyl)-N-((3aS,4R,7S,7aR)-2,2,4-trimethyltetrahydro-4H-[1,3]dioxolo[4,5-c]pyran-7-yl)pyrazin-2-amine as colorless oil. Yield: 60%. LCMS found: [M+H]⁺=334.
Step 3: To a solution of 6-(trifluoromethyl)-N-((3aS,4R,7S,7aR)-2,2,4-trimethyltetrahydro-4H-[1,3]dioxolo[4,5-c]pyran-7-yl)pyrazin-2-amine (1.00 eq) in THE was added HCl (1 mL, 2 N) at 0° C. The mixture was stirred at 25° C. for 16 hours, then concentrated. The residue was purified by reverse phase (C18, 5-40% MeCN in water, 0.3% TFA) to afford (2R,3R,4R,5S)-2-methyl-5-((6-(trifluoromethyl)pyrazin-2-yl)amino)tetrahydro-2H-pyran-3,4-diol (TFA salt) (A136) as white solid. Yield: 55%. LCMS found: [M+H]⁺=294. ¹H NMR (400 MHz, CD₃OD): δ 8.10 (s, 1H), 7.99 (s, 1H), 4.38-4.21 (m, 1H), 4.07 (dd, J=11.0, 5.2 Hz, 1H), 3.71-3.68 (m, 1H), 3.63 (dd, J=10.5, 3.2 Hz, 1H), 3.57 (dd, J=6.5, 1.0 Hz, 1H), 3.10 (t, J=10.8 Hz, 1H), 1.26 (d, J=6.5 Hz, 3H).

### Preparation of (2R,3R,4R,5S)-2-(hydroxymethyl)-5-((5-methoxypyrimidin-4-yl)amino)tetrahydro-2H-pyran-3,4-diol (A156)

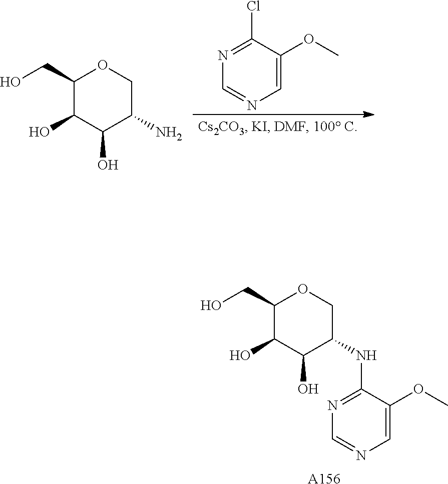

The mixture of (2R,3R,4R,5S)-5-amino-2-(hydroxymethyl)tetrahydro-2H-pyran-3,4-diol (1.00 eq), 4-chloro-5-methoxypyrimidine (1.20 eq), KI (0.2 eq) and Cs₂CO₃ (2.00 eq) in DMF was stirred at 100° C. for 12 hours. The mixture was filtered, the filtrate was concentrated under reduced pressure, the residue was purified by Prep-HPLC to afford (2R,3R,4R,5S)-2-(hydroxymethyl)-5-((5-methoxypyrimidin-4-yl)amino)tetrahydro-2H-pyran-3,4-diol (A137) as a yellow solid. Yield: 20%. Chemical Formula: C₁₁H₁₇N₃O₅. LCMS found: [M+H]⁺=272. ¹H NMR (400 MHz, CD₃OD) δ 8.08 (s, 1H), 7.70 (s, 1H), 4.52 (td, J=10.6, 5.2 Hz, 1H), 4.07 (dd, J=10.9, 5.2 Hz, 1H), 3.91 (s, 3H), 3.90-3.88 (m, 1H), 3.79-3.66 (m, 3H), 3.48-3.43 (m, 1H), 3.19 (t, J=10.8 Hz, 1H).

### Preparation of 1-(((2R,3R,4R,5S)-3,4-dihydroxy-5-((6-(trifluoromethyl) pyrazin-2-yl) amino) tetrahydro-2H-pyran-2-yl) methyl) pyrrolidin-2-one (A157)

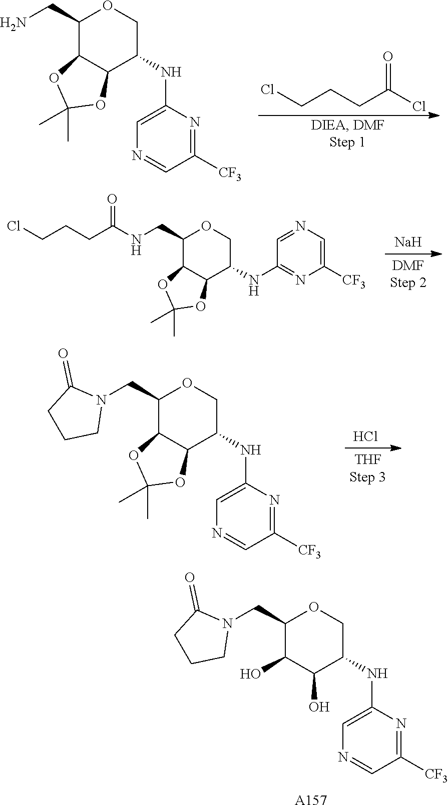

Step 1: To a solution of N-((3aS,4R,7S,7aR)-4-(aminomethyl)-2,2-dimethyltetrahydro-4H-[1,3]dioxolo[4,5-c]pyran-7-yl)-6-(trifluoromethyl)pyrazin-2-amine (1.00 eq) and DIEA (3.00 eq) in DMF (2 mL) was added 4-chlorobutanoyl chloride (1.20 eq) dropwise at 0° C. The resulting mixture was stirred at 25° C. for 2 hours. The crude mixture of 4-chloro-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)butanamide was used for next step directly. Chemical Formula: C₁₈H₂₄ClF₃N₄O₄, LCMS found: [M+H]⁺=453.
Step 2: To a solution of 4-chloro-N-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)butanamide (1.00 eq) in DMF (2 mL) was added NaH (60% purity, 10.00 eq). The mixture was stirred at 25° C. for 12 hours. The mixture was quenched with saturated solution of NH₄Cl, then extracted with ethyl acetate, the organic layer was washed with brine, dried over anhydrous sodium sulfate, filtered and concentrated under reduced pressure, the residue was purified by silica gel chromatography (PE/EA=10/1 to 1/1) to afford 1-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrrolidin-2-one as a yellow oil. Yield: 40%. Chemical Formula: C₁₈H₂₃F₃N₄O₄, LCMS found: [M+H]⁺=417.
Step 3: To a solution of 1-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)pyrrolidin-2-one (1.00 eq) in THE (3 mL) was added 2N HCl (5.00 eq). The mixture was stirred at 25° C. for 12 hours. The mixture was concentrated under reduced pressure. The residue was purified by reverse phase to afford A138 as white solid. Yield: 50%. Chemical Formula: C₁₅H₁₉F₃N₄O₄, LCMS found: [M+H]⁺=377. ¹H NMR (400 MHz, CD₃OD): δ 8.12 (s, 1H), 8.02 (s, 1H), 4.33 (td, J=10.6, 5.2 Hz, 1H) 4.13 (dd, J=11.0, 5.2 Hz, 1H), 3.83 (d, J=3.2 Hz, 1H), 3.69-3.62 (m, 3H), 3.58-3.49 (m, 2H), 3.45 (dd, J=14.0, 8.4 Hz, 1H), 3.10 (t, J=10.8 Hz, 1H), 2.40 (t, J=8.4 Hz, 2H), 2.12-2.01 (m, 2H).

### Preparation of (2R,3R,4R,5S)-2-(hydroxymethyl)-5-((3-(trifluoromethyl)cyclohexyl)amino) tetrahydro-2H-pyran-3,4-diol (A158)

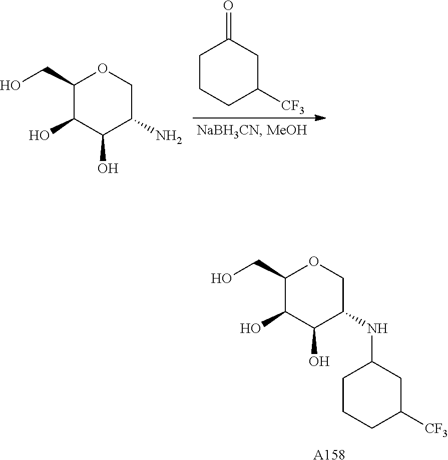 [SMILES: N[C@H]1CO[C@H](CO)[C@H](O)[C@@H]1O ; O=C1CCCC(C(F)(F)F)C1 ; OC[C@H]1OC[C@H](NC2CCCC(C(F)(F)F)C2)[C@@H](O)[C@H]1O ; "A158"]

To a solution of (2R,3R,4R,5S)-5-amino-2-(hydroxymethyl)tetrahydro-2H-pyran-3,4-diol (1.00 eq) and 3-(trifluoromethyl)cyclohexan-1-one (1.50 eq) in MeOH (2 mL) was added NaBH₃CN (2.00 eq) at 25° C. The reaction was stirred at 50° C. for 10 hours. The mixture was concentrated and the residue was purified by reverse phase (C18, 0-40% MeCN in water, 0.3% TFA) to afford 1-(((2R,3R,4R,5S)-3,4-dihydroxy-5-((6-(trifluoromethyl)pyrazin-2-yl)amino)tetrahydro-2H-pyran-2-yl)methyl)pyrrolidin-2-one as white solid. Yield: 20.0%. ¹H NMR (400 MHz, CD₃OD): δ 4.29-4.15 (m, 1H), 3.93 (t, J=3.6 Hz, 1H), 3.78-3.72 (m, 2H), 3.71-3.61 (m, 2H), 3.60-3.51 (m, 1H), 3.49-3.44 (m, 1H), 3.43-3.36 (m, 1H), 2.77-2.27 (m, 2H), 2.23-2.20 (m, 1H) 2.06-1.94 (m, 2H), 1.83-1.70 (m, 2H), 1.57-1.44 (m, 1H), 1.43-1.22 (m, 1H).

### Preparation of (2R,3R,4R,5S)-5-(cyclohexylamino)-2-(hydroxymethyl)tetrahydro-2H-pyran-3,4-diol (A159)

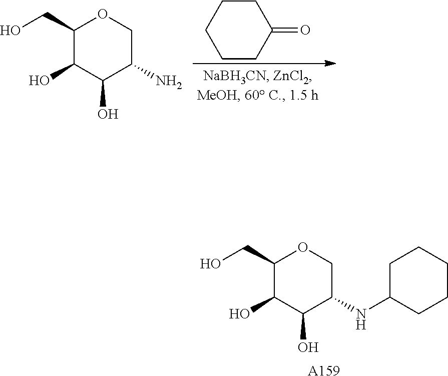

To a solution of (2R,3R,4R,5S)-5-amino-2-(hydroxymethyl)tetrahydro-2H-pyran-3,4-diol (200 mg, 1.00 eq) and cyclohexanone (240 mg, 2.00 eq) in MeOH (5 mL) was added ZnCl₂ (830 mg, 5.00 eq), the mixture was stirred at 25° C. for 0.5 h, then NaBH₃CN (380 mg, 5.00 eq) was added, the resulting mixture was stirred at 60° C. for 1 h. After completion, the mixture was quenched with water and concentrated under reduced pressure, the residue was purified by Mass-Guided to give (2R,3R,4R,5S)-5-(cyclohexylamino)-2-(hydroxymethyl)tetrahydro-2H-pyran-3,4-diol (40 mg, formate) as yellow solid. Yield: 13.3%. Chemical Formula: C₁₂H₂₃NO₄, LCMS found: [M+H]⁺=246, ¹H NMR (400 MHz, DMSO-d₆) δ 8.25 (brs, 1H), 4.65-4.45 (m, 2H), 3.92 (d, J=7.3 Hz, 1H), 3.82-3.60 (m, 2H), 3.51-3.35 (m, 4H), 3.24 (t, J=6.0 Hz, 1H), 3.13-2.96 (m, 2H), 2.83-2.71 (m, 1H), 1.93 (d, J=11.8 Hz, 1H), 1.82 (d, J=8.8 Hz, 1H), 1.78-1.65 (m, 2H), 1.56 (d, J=12.2 Hz, 1H), 1.25-1.02 (m, 5H).

### Preparation of (2R,3R,4R,5S)-5-(bis(cyclopropylmethyl)amino)-2-(hydroxymethyl) tetrahydro-2H-pyran-3,4-diol (A160)

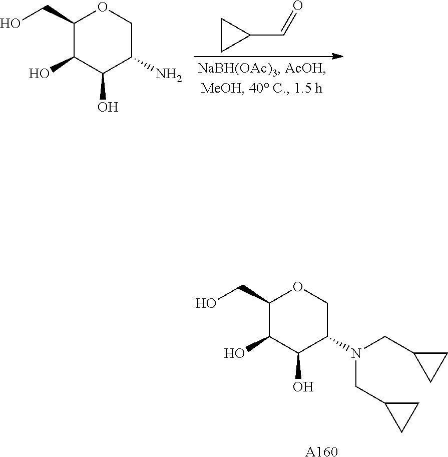 [SMILES: N[C@H]1CO[C@H](CO)[C@H](O)[C@@H]1O ; O=CC1CC1 ; OC[C@H]1OC[C@H](N(CC2CC2)CC2CC2)[C@@H](O)[C@H]1O ; "A160"]

To a solution of (2R,3R,4R,5S)-5-amino-2-(hydroxymethyl)tetrahydro-2H-pyran-3,4-diol (50 mg, 1.00 eq) and cyclopropanecarbaldehyde (43 mg, 2.00 eq) in MeOH (5 ml) was added AcOH (1.00 eq), the mixture was stirred at 25° C. for 0.5 h, then NaBH(OAc)₃ (325 mg, 5.00 eq) was added, the resulting mixture was stirred at 40° C. for 1 h. After completion, the mixture was quenched with water, then concentrated under reduced pressure, the residue was purified by Mass-Guided to give (2R,3R,4R,5S)-5-(bis(cyclopropylmethyl)amino)-2-(hydroxymethyl)tetrahydro-2H-pyran-3,4-diol (7.0 mg, formate) as brown solid. Yield: 8.4%. Chemical Formula: C₁₄H₂₅NO₄, LCMS found: [M+H]⁺=272, ¹H NMR (400 MHz, CD₃OD) δ 4.19 (dd, J=10.9, 4.1 Hz, 1H), 4.10 (td, J=10.6, 4.1 Hz, 1H), 4.05-3.94 (m, 2H), 3.75-3.64 (m, 2H), 3.57 (t, J=10.8 Hz, 1H), 3.47 (t, J=5.9 Hz, 1H), 3.36-3.32 (m, 2H), 3.16-3.04 (m, 2H), 1.24-1.11 (m, 2H), 0.85-0.72 (m, 4H), 0.50-0.43 (m, 4H).

### Preparation of (2R,3R,4S,5S)-2-(hydroxymethyl)-5-((6-(trifluoromethyl)pyridin-2-yl)thio)tetrahydro-2H-pyran-3,4-diol (A161)

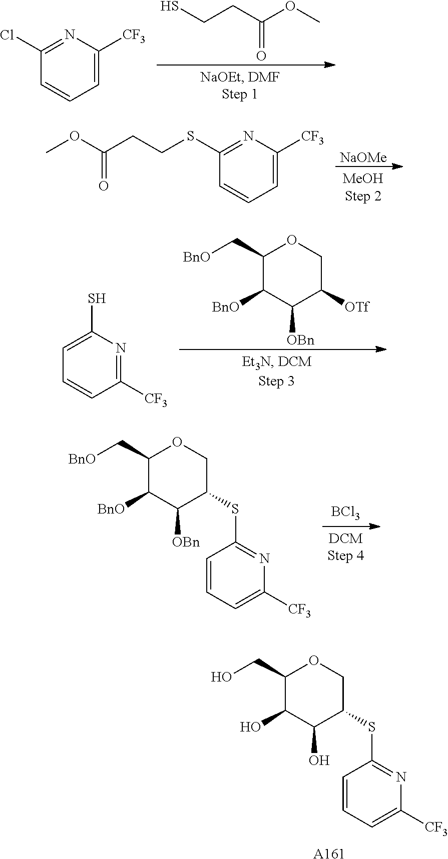

Step 1: To a solution of 2-chloro-6-(trifluoromethyl)pyridine (1.00 eq) and methyl 3-mercaptopropanoate (2.00 eq) in DMF (20 mL) was added NaOEt (1.00 eq) at 25° C. The reaction was stirred at 80° C. for 16 hours. LCMS showed the desired mass was detected. The mixture was quenched with water and extracted with ethyl acetate, the organic layer was concentrated and purified by flash chromatography (PE/EA=20/1 to 5/1) to give methyl 3-((6-(trifluoromethyl)pyridin-2-yl)thio)propanoate as a yellow oil. Yield: 27%. LC-MS (ESI) found: [M+H]⁺=266.0.
Step 2: To a solution of methyl 3-((6-(trifluoromethyl)pyridin-2-yl)thio)propanoate (1.00 eq) in MeOH (10 mL) was added NaOMe (1.20 eq) dropwise at 20° C. The mixture was stirred at 80° C. for 2 hours. TLC showed one new spot was detected. The mixture was concentrated and the residue was purified by silica gel chromatography (PE/EA=10/1 to 4/1) to give 6-(trifluoromethyl)pyridine-2-thiol as yellow solid. Yield: 37%. LCMS found: [M+H]⁺=180.0.
Step 3: To a solution of 6-(trifluoromethyl)pyridine-2-thiol (1.00 eq) and Et₃N (3.00 eq) in DCM (10 mL) was added (3R,4S,5S,6R)-4,5-bis(benzyloxy)-6-((benzyloxy)methyl)tetrahydro-2H-pyran-3-yl trifluoromethanesulfonate (1.50 eq) at 0° C. The reaction was stirred at 20° C. for 16 hours. The desired mass was observed by LCMS. The mixture was concentrated and then purified by flash chromatography (PE/EA=20/1 to 5/1) to give 2-(((3S,4S,5S,6R)-4,5-bis(benzyloxy)-6-((benzyloxy)methyl)tetrahydro-2H-pyran-3-yl)thio)-6-(trifluoromethyl)pyridine as a colorless oil. Yield: 13%. LC-MS (ESI) found: 596.2 [M+H]⁺.
Step 4: To a solution of 2-(((3S,4S,5S,6R)-4,5-bis(benzyloxy)-6-((benzyloxy)methyl)tetrahydro-2H-pyran-3-yl)thio)-6-(trifluoromethyl)pyridine (1.00 eq) in DCM (5 mL) was added BCl₃ (10.0 eq) dropwise at −20° C., the mixture was warmed to 20° C. and stirred for 12 hours. The mixture was quenched with Et₃N, then concentrated under reduced pressure, the residue was purified by reverse phase (5-45% MeOH in H₂O, 0.1% TFA) to give (2R,3R,4S,5S)-2-(hydroxymethyl)-5-((6-(trifluoromethyl)pyridin-2-yl)thio)tetrahydro-2H-pyran-3,4-diol (TFA salt) as a colorless oil. Yield: 34%. LCMS found: [M+H]⁺=326.1. ¹H NMR (400 MHz, CD₃OD): δ 7.78 (t, J=7.6 Hz, 1H), 7.51 (d, J=7.6 Hz, 1H), 7.47 (d, J=7.6 Hz, 1H), 4.28 (dd, J=11.2, 5.2 Hz, 1H), 4.17 (td, J=11.2, 5.2 Hz, 1H), 3.93-3.87 (m, 1H), 3.76 (dd, J=11.2, 7.2 Hz, 1H), 3.71-3.61 (m, 2H), 3.51-3.47 (m, 1H), 3.43 (t, J=11.2 Hz, 1H).

### Preparation of (2S,3R,4R,5S)-2-(fluoromethyl)-5-((6-(trifluoromethyl)pyrazin-2-yl)amino)tetrahydro-2H-pyran-3,4-diol (A162)

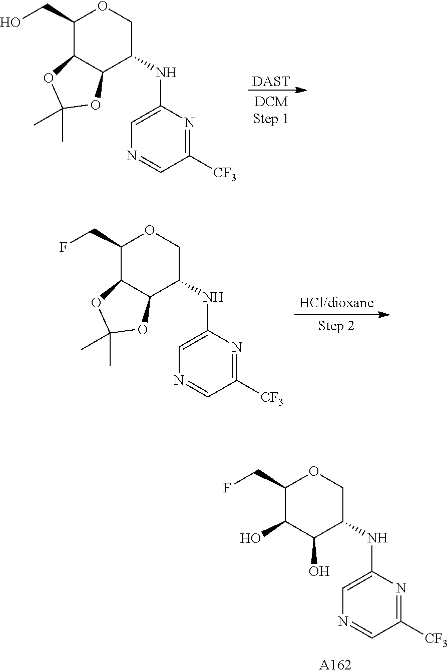 [SMILES: CC1(C)O[C@@H]2[C@H](O1)[C@@H](Nc1cncc(C(F)(F)F)n1)CO[C@@H]2CO ; "DAST" ; CC1(C)O[C@@H]2[C@H](O1)[C@@H](Nc1cncc(C(F)(F)F)n1)CO[C@@H]2CF ; "HCl/dioxane" ; O[C@@H]1[C@H](O)[C@@H](Nc2cncc(C(F)(F)F)n2)CO[C@@H]1CF ; "A162"]

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methanol (1.00 eq) in DCM (2 mL) was added DAST (2.00 eq) dropwise at 0° C. The reaction was stirred at 40° C. for 2 hours. LCMS showed the desired mass was detected. The mixture was diluted with DCM, washed with H₂O and brine, the organic layer was dried over Na₂SO₄, filtered and concentrated in vacuo. The crude product was purified by flash chromatography (silica gel, 0˜80% EA in PE) to give N-((3aR,4S,7S,7aR)-4-(fluoromethyl)-2,2-dimethyltetrahydro-4H-[1,3]dioxolo[4,5-c]pyran-7-yl)-6-(trifluoromethyl)pyrazin-2-amine as a colorless oil. Yield: 19.9%. LC-MS (ESI) found: 352 [M+H]⁺.
Step 2: The solution of N-((3aR,4S,7S,7aR)-4-(fluoromethyl)-2,2-dimethyltetrahydro-4H-[1,3]dioxolo[4,5-c]pyran-7-yl)-6-(trifluoromethyl)pyrazin-2-amine (1.00 eq) in HCl/1,4-dioxane (1 mL) was stirred at 25° C. for 1 hour. LCMS showed the desired mass was detected. The mixture was concentrated in vacuo. The crude product was purified by prep-HPLC to give (2S,3R,4R,5S) 2-(fluoromethyl)-5-((6-(trifluoromethyl)pyrazin-2-yl)amino)tetrahydro-2H-pyran-3,4-diol as a yellow solid. Yield: 43.3%. LCMS found: [M+H]⁺=312. ¹H NMR (400 MHz, CD₃OD): δ 8.12 (s, 1H), 8.01 (s, 1H), 4.64-4.59 (m, 1H), 4.52-4.46 (m, 1H), 4.34 (td, J=10.4, 5.2 Hz, 1H), 4.14 (dd, J=11.2, 5.2 Hz, 1H), 3.92 (dd, J=3.2, 0.8 Hz, 1H), 3.78-3.70 (m, 1H), 3.67 (d, J=10.4, 3.2 Hz, 1H), 3.15 (t, J=10.8 Hz, 1H).

### Preparation of (2R,3R,4R,5S)-2-(hydroxymethyl)-5-(isopropylamino)tetrahydro-2H-pyran-3,4-diol (A163)

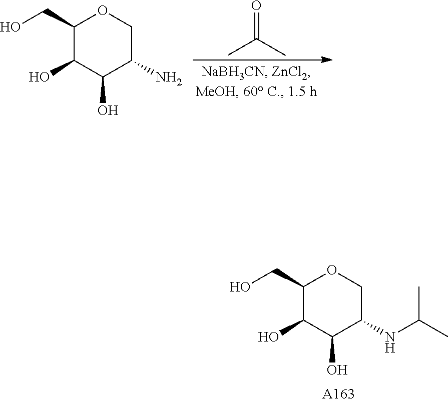

To a solution of (2R,3R,4R,5S)-5-amino-2-(hydroxymethyl)tetrahydro-2H-pyran-3,4-diol (200 mg, 1.00 eq) and acetone (140 mg, 2.00 eq) in MeOH (5 mL) was added ZnCl₂ (830 mg, 5.00 eq), the mixture was stirred at 25° C. for 0.5 h, then NaBH₃CN (380 mg, 5.00 eq) was added, the resulting mixture was stirred at 60° C. for 1 h. The mixture was quenched with water and then concentrated under reduced pressure, the residue was purified by Mass-Guided to give (2R,3R,4R,5S)-2-(hydroxymethyl)-5-(isopropylamino)tetrahydro-2H-pyran-3,4-diol (60 mg, formate) as yellow solid. Yield: 23.9%. Chemical Formula: C₉H₁₉NO₄, LCMS found: [M+H]⁺=206, 1H NMR (400 MHz, CD₃OD) δ 4.21 (dd, J=9.7, 3.5 Hz, 1H), 3.90 (d, J=2.7 Hz, 1H), 3.76-3.54 (m, 4H), 3.50-3.35 (m, 3H), 1.39-1.33 (m, 6H).

### Preparation of (2R,3R,4R,5S)-2-(hydroxymethyl)-5-((6-methoxypyridin-2-yl)amino) tetrahydro-2H-pyran-3,4-diol (A164)

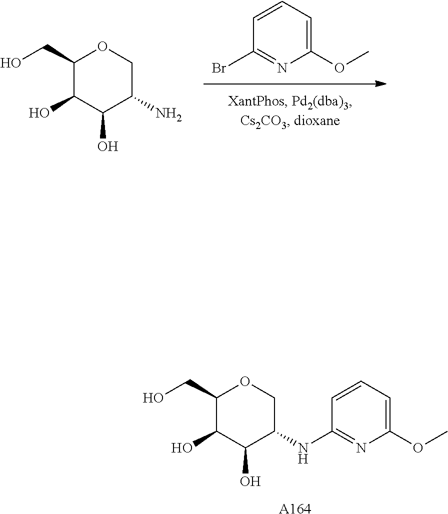

To a solution of (2R,3R,4R,5S)-5-amino-2-(hydroxymethyl)tetrahydro-2H-pyran-3,4-diol (1.00 eq) and 2-bromo-6-methoxypyridine (1.20 eq) in dioxane was added XantPhos (0.2 eq), Pd₂(dba)₃ (0.05 eq) and Cs₂CO₃ (2.00 eq), the mixture was degassed and purged with nitrogen for several times, then stirred at 100° C. for 16 hours under N₂ atmosphere. The mixture was treated with metal scavenger, then filtered, the filtrate was concentrated and the residue was purified by Prep-HPLC to afford (2R,3R,4R,5S)-2-(hydroxymethyl)-5-((6-methoxypyridin-2-yl)amino)tetrahydro-2H-pyran-3,4-diol as a white solid. Yield: 0.6%. Chemical Formula: C₁₂H₁₈N₂O₅. LCMS found: [M+H]⁺=271. ¹H NMR (400 MHz, CD₃OD): δ 7.29 (t, J=7.9 Hz, 1H), 6.08 (d, J=7.9 Hz, 1H) 5.93 (d, J=7.9 Hz, 1H), 4.22-4.13 (m, 2H), 3.89 (d, J=2.8 Hz, 1H), 3.83 (s, 3H), 3.76 (dd, J=11.2, 7.2 Hz, 1H), 3.69 (dd, J=11.2, 5.2 Hz, 1H), 3.56 (dd, J=9.8, 3.2 Hz, 1H), 3.47-3.41 (m, 1H), 3.11 (t, J=12.0 Hz, 1H).

### Preparation of (2R,3R,4R,5S)-2-((4-((1-(4-(aminomethyl)phenyl)piperidin-4-yl)methyl)piperazin-1-yl)methyl)-5-((6-(trifluoromethyl)pyrazin-2-yl)amino)tetrahydro-2H-pyran-3,4-diol (A165)

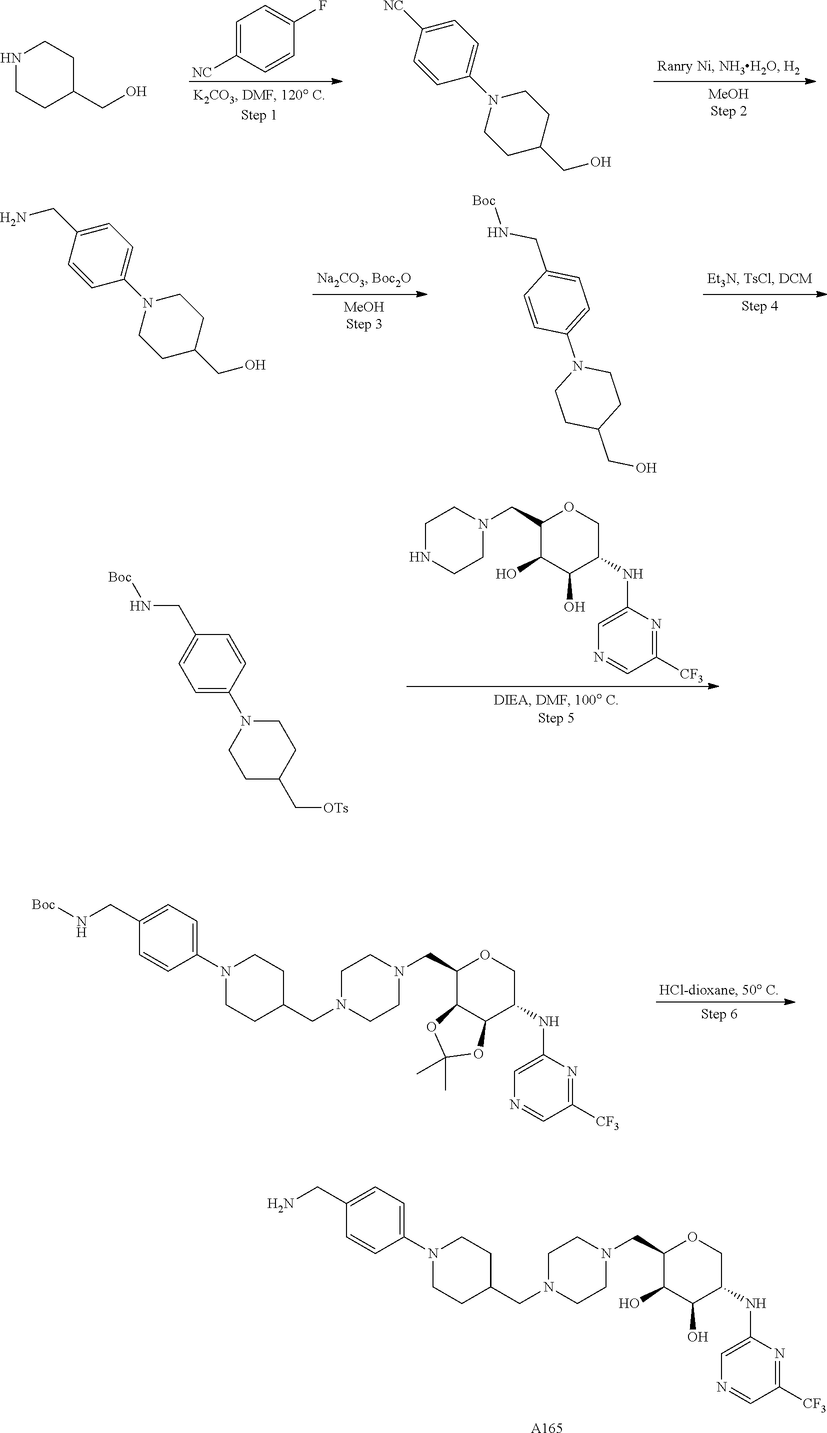

Step 1: To a solution of piperidin-4-ylmethanol (1.00 eq) and 4-fluorobenzonitrile (1.50 eq) in DMF (10 mL) was added K₂CO₃ (3.00 eq) at 25° C. The reaction was stirred at 120° C. for 5 hours. LCMS showed the desired mass was detected. The mixture was diluted with water, then extracted with ethyl acetate, the organic layer was washed with brine, dried over anhydrous sodium sulfate, filtered and concentrated in vacuum. The crude product was purified by flash chromatography (silica gel, 0˜50% EA in PE) to give 4-(4-(hydroxymethyl)piperidin-1-yl)benzonitrile as a white solid. Yield: 81.7%. LC-MS (ESI) found: 217 [M+H]⁺.
Step 2: To a solution of 4-(4-(hydroxymethyl)piperidin-1-yl)benzonitrile (1.00 eq) in MeOH (20 mL) was added NH₃·H₂O (0.30 eq) and Raney Ni (0.1 eq) at 25° C. The mixture was stirred at 25° C. for 12 hours under H2 atmosphere (15 psi). LCMS showed the desired mass was detected. The resulting mixture was filtered, the filtrate was concentrated in vacuum to give (1-(4-(aminomethyl)phenyl)piperidin-4-yl)methanol (crude) as a colorless oil, which was used for next step without further purification. LCMS found: [M+H]⁺=221.
Step 3: To a solution of (1-(4-(aminomethyl)phenyl)piperidin-4-yl)methanol (1.00 eq) in MeOH (20 mL) was added Na₂CO3 (3.00 eq) and Boc₂O (2.00 eq) at 25° C. The reaction was stirred at 25° C. for 12 hours. LCMS showed the desired mass was detected. The mixture was diluted with water, then extracted with ethyl acetate, the organic layer was dried over anhydrous sodium sulfate, filtered and concentrated in vacuum. The crude product was purified by flash chromatography (silica gel, 0˜10% MeOH in DCM) to give tert-butyl (4-(4-(hydroxymethyl)piperidin-1-yl)benzyl)carbamate as a white solid. Yield: 31.4%. LC-MS (ESI) found: 321 [M+H]⁺.
Step 4: To a solution of tert-butyl (4-(4-(hydroxymethyl)piperidin-1-yl)benzyl)carbamate (1.00 eq) in DCM (20 mL) was added Et₃N (3.00 eq), TsCl (1.30 eq) at 25° C. The reaction was stirred at 25° C. for 12 hours. LCMS showed the desired mass was detected. The mixture was diluted with water and extracted with ethyl acetate, the organic layer was dried over anhydrous sodium sulfate, filtered and concentrated in vacuum. The crude product was purified by flash chromatography (silica gel, 0˜40% EA in PE) to give (1-(4-(((tert-butoxycarbonyl)amino)methyl)phenyl)piperidin-4-yl)methyl 4-methylbenzenesulfonate as a white solid. Yield: 27.6%. LC-MS (ESI) found: 475 [M+H]⁺.
Step 5: To a solution of (1-(4-(((tert-butoxycarbonyl)amino)methyl)phenyl)piperidin-4-yl)methyl 4-methylbenzenesulfonate (1.50 eq) and (2R,3R,4R,5S)-2-(piperazin-1-ylmethyl)-5-((6-(trifluoromethyl)pyrazin-2-yl)amino)tetrahydro-2H-pyran-3,4-diol (1.00 eq) in DMF (20 mL) was added DIEA (3.00 eq) at 25° C. The reaction was stirred at 100° C. for 5 hours. LCMS showed the desired mass was detected. The mixture was diluted with water and extracted with ethyl acetate, the organic layer was washed with brine, dried over anhydrous sodium sulfate, filtered and concentrated in vacuum. The crude product was purified by reverse phase (C18, 5-40% MeCN in water, 0.1% TFA) to give tert-butyl (4-(4-((4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)piperazin-1-yl)methyl)piperidin-1-yl)benzyl)carbamate as a yellow oil. Yield: 72.6%. LC-MS (ESI) found: 720 [M+H]⁺.
Step 6: A solution of tert-butyl (4-(4-((4-(((3aS,4R,7S,7aR)-2,2-dimethyl-7-((6-(trifluoromethyl)pyrazin-2-yl)amino)tetrahydro-4H-[1,3]dioxolo[4,5-c]pyran-4-yl)methyl)piperazin-1-yl)methyl)piperidin-1-yl)benzyl)carbamate (1.00 eq) in HCl/dioxane (10 mL, 4 N) was stirred at 50° C. for 0.5 hour. LCMS showed the desired mass was detected. The mixture was concentrated under reduced pressure, the residue was purified by reverse phase (C18, 5-30% MeCN in water, 0.1% TFA) to give (2R,3R,4R,5S)-2-((4-((1-(4-(aminomethyl)phenyl)piperidin-4-yl)methyl)piperazin-1-yl)methyl)-5-((6-(trifluoromethyl)pyrazin-2-yl)amino)tetrahydro-2H-pyran-3,4-diol as a yellow oil. Yield: 81.0%. LC-MS (ESI) found: 580 [M+H]⁺. ¹H NMR (400 MHz, CD₃OD): δ 8.13 (s, 1H), 8.02 (s, 1H), 7.25 (d, J=8.6 Hz, 2H), 6.61 (d, J=8.8 Hz, 2H), 4.35 (td, J=10.4, 5.0 Hz, 1H), 4.21-4.14 (m, 1H), 3.99-3.97 (m, 3H), 3.90 (d, J=2.8 Hz, 1H), 3.69-3.59 (m, 10H), 3.58-3.46 (m, 2H), 3.42-3.41 (m, 2H), 3.33-3.31 (m, 1H), 3.29-3.26 (m, 1H), 3.21 (t, J=10.8 Hz, 1H), 3.03-2.97 (m, 1H), 2.39-2.37 (m, 1H), 2.28-2.20 (m, 1H), 1.98-1.94 (m, 2H), 1.79-1.67 (m, 1H).

### Preparation of (2R,3R,4R,5S)-5-(cyclobutylamino)-2-(hydroxymethyl)tetrahydro-2H-pyran-3,4-diol (A166)

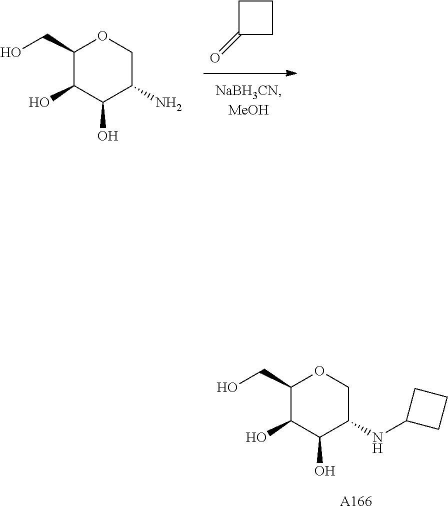

To a solution of (2R,3R,4R,5S)-5-amino-2-(hydroxymethyl)tetrahydro-2H-pyran-3,4-diol (1.00 eq) and cyclobutanone (1.30 eq) in MeOH (50 mL) was added NaBH₃CN (3.00 eq). The reaction was stirred at 25° C. for 2 hours. LCMS showed the desired mass was detected. The mixture was concentrated and the residue was purified by Mass-Guided LC-MS to give (2R,3R,4R,5S)-5-(cyclobutylamino)-2-(hydroxymethyl)tetrahydro-2H-pyran-3,4-diol (Formate) as a yellow oil. Yield: 31.5%. LC-MS (ESI) found: 218 [M+H]⁺. ¹H NMR (400 MHz, CD₃OD): δ 8.38 (brs, 1H) 4.16 (dd, J=10.8, 4.8 Hz, 1H), 3.94-3.85 (m, 2H), 3.76-3.56 (m, 3H), 3.46-3.40 (m, 1H) 3.36 (t, J=10.8 Hz, 1H), 3.29-3.22 (m, 1H), 2.44-2.28 (m, 2H), 2.27-2.04 (m, 2H), 1.97-1.82 (m, 2H).

### Preparation of (3S,4R,5S)-1-methyl-5-((6-(trifluoromethyl)pyrazin-2-yl)amino)piperidine-3,4-diol (A167)

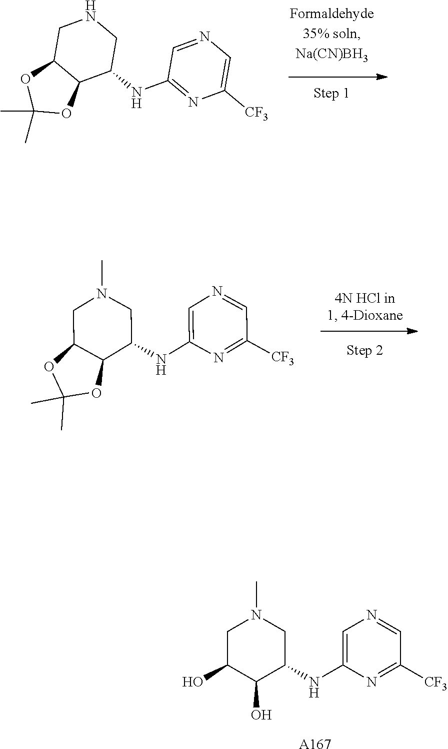 [SMILES: CC1(C)O[C@@H]2[C@@H](Nc3cncc(C(F)(F)F)n3)CNC[C@@H]2O1 ; CN1C[C@H](Nc2cncc(C(F)(F)F)n2)[C@H]2OC(C)(C)O[C@H]2C1 ; CN1C[C@H](Nc2cncc(C(F)(F)F)n2)[C@@H](O)[C@@H](O)C1 ; "A167"]

Step 1: To a solution of (3aS,7S,7aR)-2,2-dimethyl-N-(6-(trifluoromethyl)pyrazin-2-yl)hexahydro-[1,3]dioxolo[4,5-c]pyridin-7-amine (60 mg, 0.188 mmol) in DCM:MeOH (1:1, 2 mL) were added 35% aq. formaldehyde (0.05 mL, 0.376 mmol) and acetic acid (0.1 mL) at 0° C. The reaction mixture was stirred at rt for 2 h. Reaction mixture was cooled to 0° C., added Na(CN)BH₃ (36 mg, 0.564 mmol) in portions. The reaction mixture was stirred at rt for 12 h. Reaction mixture was cooled to 0° C., added saturated aq. NaHCO₃ solution (10 mL) and extracted with DCM (2×50 mL). Combined organic layer was dried and concentrated to give (3aS,7S,7aR) 2,2,5-trimethyl-N-(6-(trifluoromethyl)pyrazin-2-yl)hexahydro-[1,3]dioxolo[4,5-c]pyridin-7-amine (50 mg, crude). This was used for next step without purification. LC-MS (ESI) found: 333.6 [M+H]⁺ (82% of desired mass).
Step-2: To a solution of (3aS,7S,7aR)-2,2,5-trimethyl-N-(6-(trifluoromethyl)pyrazin-2-yl)hexahydro-[1,3]dioxolo[4,5-c]pyridin-7-amine (50 mg, crude) in DCM (4.0 mL) was added 4N HCl in 1, 4-Dioxane (0.5 mL) at 0° C. The reaction mixture was stirred at rt for 1 h. After completion of the starting material, removed all the volatiles under reduced pressure to give crude material which was purified by prep-HPLC to give (3S,4R,5S)-5-((6-(trifluoromethyl)pyrazin-2-yl)amino)piperidine-3,4-diol (10 mg, 25% yield). 1H NMR (400 MHz, DMSO-d₆): δ 8.28 (s, 1H), 8.02 (s, 1H), 7.60 (d, J=7.34 Hz, 1H), 5.96 (s, 2H), 4.03 (s, 1H), 3.73 (s, 1H), 3.48 (s, 2H) 2.59 (s, 1H), 2.30 (s, 2H), 2.12-2.18 (m, 3H). LC-MS (ESI) found: 293.2 [M+H]⁺.

### Preparation of (3S,4R,5S)-5-(methyl(6-(trifluoromethyl)pyrazin-2-yl)amino)piperidine-3,4-diol (A168) and (3S,4R,5S)-1-ethyl-5-(methyl(6-(trifluoromethyl)pyrazin-2-yl)amino) piperidine-3,4-diol (A169)

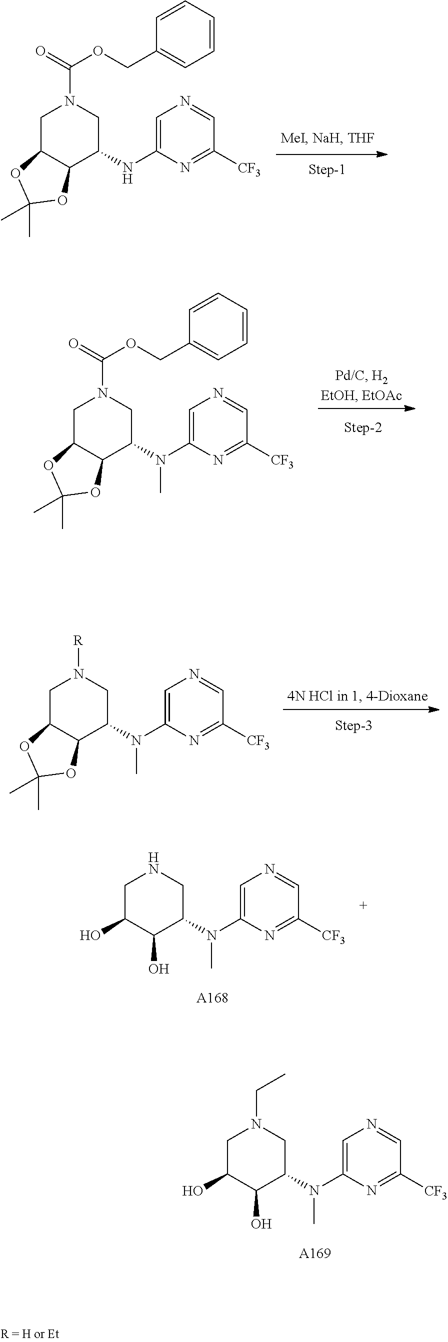

Step 1: To a solution of benzyl (3aS,7S,7aR)-2,2-dimethyl-7-((6-(trifluoromethyl)pyrazin-2-yl)amino)tetrahydro-[1,3]dioxolo[4,5-c]pyridine-5(4H)-carboxylate (150 mg, 0.33 mmol) in THE (6.0 mL) was added NaH (20 mg, 0.49 mmol) followed by Mel (70 mg, 0.49 mmol, 50% in THE stock solution) at 0° C. The reaction mixture was stirred at rt for 16 h. After completion of starting material, added saturated aq. NaHCO₃ (50 mL) and extracted with EtOAc (2×50 mL). Combined organic layer was dried and concentrated to give crude product which was purified by column chromatography eluted with 1-5% (MeOH:DCM) to give benzyl (3aS,7S,7aR)-2,2-dimethyl-7-(methyl(6-(trifluoromethyl)pyrazin-2-yl)amino)tetrahydro-[1,3]dioxolo[4,5-c]pyridine-5(4H)-carboxylate (100 mg, 77% yield). ¹H NMR (400 MHz, DMSO-d₆) δ 8.44 (s, 1H) 8.24 (s, 1H) 7.24-7.42 (m, 5H) 5.09 (s, 2H) 4.45 (dd, J=8.56, 5.14 Hz, 1H) 4.22-4.33 (m, 3H) 3.75-3.84 (m, 1H) 3.05 (s, 3H) 1.38 (s, 2H) 1.24 (s, 6H). LC-MS (ESI) found: 467.2 [M+H]⁺.
Step 2: To a solution of benzyl (3aS,7S,7aR)-2,2-dimethyl-7-(methyl(6-(trifluoromethyl)pyrazin-2-yl)amino)tetrahydro-[1,3]dioxolo[4,5-c]pyridine-5(4H)-carboxylate (100 mg, 0.21 mmol) in EtOH:EtOAc (2.5 mL and 2.5 mL) was added 10% Pd/C (20 mg) at rt. The reaction mixture was stirred under H2 atmosphere (balloon pressure) at rt for 16 h. After completion of the starting material, reaction mixture was filtered through celite pad and the filtrate was concentrated under reduced pressure to give mixture of (3aS,7S,7aR)-N,2,2-trimethyl-N-(6-(trifluoromethyl)pyrazin-2-yl)hexahydro-[1,3]dioxolo [4,5-c]pyridin-7-amine and (3aS,7S,7aR)-5-ethyl-N,2,2-trimethyl-N-(6-(trifluoromethyl)pyrazin-2-yl)hexahydro-[1,3]dioxolo[4,5-c]pyridin-7-amine (70 mg, crude). This was used for next step without purification. LC-MS: 10% of desired mass, m/z: 333 and 29% of desired mass, m/z: 361.
Step 3: To a stirred solution of (3aS,7S,7aR)-N,2,2-trimethyl-N-(6-(trifluoromethyl)pyrazin-2-yl)hexahydro-[1,3]dioxolo [4,5-c]pyridin-7-amine and (3aS,7S,7aR)-5-ethyl-N,2,2-trimethyl-N-(6-(trifluoromethyl)pyrazin-2-yl)hexahydro-[1,3]dioxolo[4,5-c]pyridin-7-amine (70 mg, crude) in DCM (4.0 mL) was added 4N HCl in 1,4-Dioxane (0.5 mL) at 0° C. The reaction mixture was stirred at rt for 1 h. After completion of the starting material, all the volatiles were removed under reduced pressure to give crude product which was purified by prep-HPLC to give (3S,4R,5S)-5-(methyl(6-(trifluoromethyl)pyrazin-2-yl)amino)piperidine-3,4-diol (10 mg, 13% yield for two steps) and (3S,4R,5S)-1-ethyl-5-(methyl(6-(trifluoromethyl)pyrazin-2-yl)amino)piperidine-3,4-diol (10 mg, 12% yield for two steps).

### Preparation of (2R,3R,4R,5S)-5-((cyclopropylmethyl)amino)-2-(hydroxymethyl)tetrahydro-2H-pyran-3,4-diol (A170)

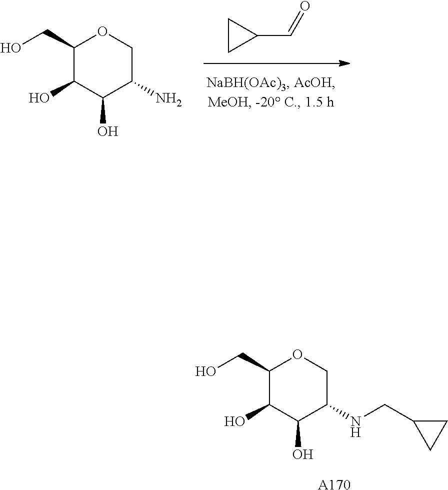

To a solution of (2R,3R,4R,5S)-5-amino-2-(hydroxymethyl)tetrahydro-2H-pyran-3,4-diol (100 mg, 1.00 eq) and cyclopropanecarbaldehyde (21 mg, 0.50 eq) in MeOH (5 ml) was added AcOH (1.00 eq), the mixture was stirred at −20° C. for 0.5 h, then NaBH(OAc)₃ (260 mg, 2.00 eq) was added, the resulting mixture was stirred at −20° C. for 1 h. LCMS showed the desired mass was observed. The mixture was quenched with water, then concentrated in vacuo, the residue was purified by Mass-Guided to give (2R,3R,4R,5S)-5-((cyclopropylmethyl)amino)-2-(hydroxymethyl)tetrahydro-2H-pyran-3,4-diol (20 mg, formate) as brown solid. Yield: 15%. LCMS found: [M+H]⁺=218.3, ¹H NMR (400 MHz, DMSO-d₆) δ 8.23 (brs, 1H), 3.92 (dd, J=10.4, 4.4 Hz, 1H), 3.66 (d, J=2.8 Hz, 1H), 3.45 (dd, J=6.0, 3.2 Hz, 2H), 3.30 (dd, J=9.8, 3.2 Hz, 1H), 3.22 (t, J=6.0 Hz, 1H), 2.98-2.82 (m, 2H), 2.56 (dd, J=12.0, 6.8 Hz, 1H), 2.48-2.44 (m, 1H), 0.94-0.84 (m, 1H), 0.48-0.39 (m, 2H), 0.17-0.11 (m, 2H).

### Preparation of (2S,3R,4R,5S)-2-(difluoromethyl)-5-((6-(trifluoromethyl)pyrazin-2-yl)amino)tetrahydro-2H-pyran-3,4-diol (A171)

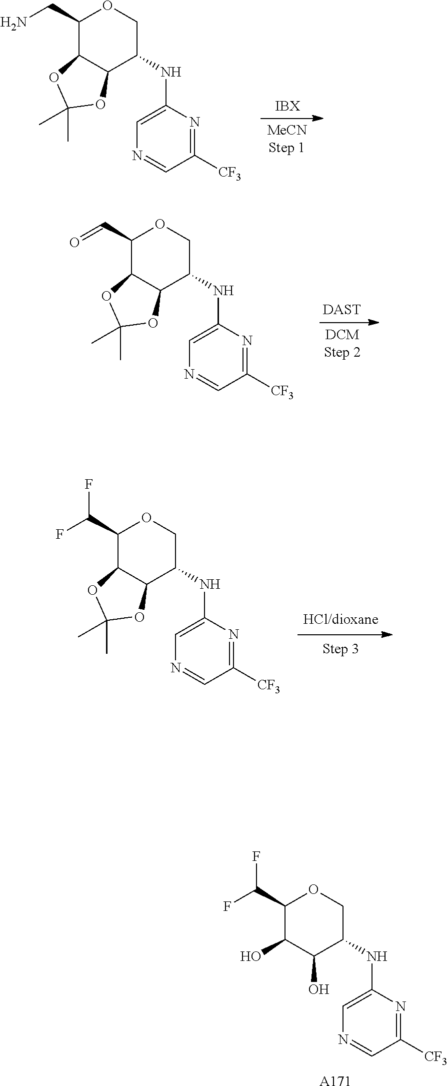

Step 1: To a solution of ((3aR,4R,7S,7aR)-2,2-dimethyl-7-((6-(trifluoromethyl)pyrazin-2-yl)amino)tetrahydro-4H-[1,3]dioxolo[4,5-c]pyran-4-yl)methanol (1.00 eq) in MeCN (2 mL) was added IBX (2.00 eq) at 25° C. The reaction was stirred at 80° C. for 1 hour. LCMS showed the desired mass was detected. The resulting mixture was filtered and concentrated in vacuo to give crude (3aR,4S,7S,7aR)-2,2-dimethyl-7-((6-(trifluoromethyl)pyrazin-2-yl)amino)tetrahydro-4H-[1,3]dioxolo[4,5-c]pyran-4-carbaldehyde as a colorless oil. LC-MS (ESI) found: 348 [M+H]⁺.
Step 2: To a solution of (3aR,4S,7S,7aR)-2,2-dimethyl-7-((6-(trifluoromethyl)pyrazin-2-yl)amino)tetrahydro-4H-[1,3]dioxolo[4,5-c]pyran-4-carbaldehyde (1.00 eq) in DCM (2 mL) was added DAST (5.00 eq) dropwise at −78° C. The reaction was stirred at 25° C. for 12 hours. LCMS showed the desired mass was detected. Then the mixture was cooled down to −30° C. and quenched with MeOH (1 mL). The mixture was concentrated and the residue was dissolved in DCM (10 mL), washed with saturated aqueous NaHCO₃ and brine, dried with anhydrous Na₂SO₄. The organic layer was separated and concentrated in vacuo. The crude product was purified by flash chromatography (silica gel, 0˜60% EA in PE) to give N-((3aR,4S,7S,7aR)-4-(difluoromethyl)-2,2-dimethyltetrahydro-4H-[1,3]dioxolo[4,5-c]pyran-7-yl)-6-(trifluoromethyl)pyrazin-2-amine as a yellow oil. Yield: 22.6%. LC-MS (ESI) found: 370 [M+H]⁺.
Step 3: The solution of N-((3aR,4S,7S,7aR)-4-(difluoromethyl)-2,2-dimethyltetrahydro-4H-[1,3]dioxolo[4,5-c]pyran-7-yl)-6-(trifluoromethyl)pyrazin-2-amine (1.00 eq) in HCl/1,4-dioxane (1 mL, 4 N) was stirred at 25° C. for 1 hour. LCMS showed the desired mass was detected. The mixture was concentrated in vacuo and the residue was purified by prep-HPLC to give (2S,3R,4R,5S)-2-(difluoromethyl)-5-((6-(trifluoromethyl)pyrazin-2-yl)amino)tetrahydro-2H-pyran-3,4-diol as a white solid. Yield: 10.1%. LCMS found: [M+H]⁺=330. ¹H NMR (400 MHz, CD₃OD): δ 8.12 (s, 1H), 8.02 (s, 1H), 5.91 (ddd, J=57.2, 54.8, 6.4 Hz, 1H), 4.37 (td, J=10.4, 4.8 Hz, 1H), 4.18 (dd, J=11.2, 5.2 Hz, 1H), 4.05-4.03 (m, 1H), 3.68 (dd, J=10.6, 3.2 Hz, 1H), 3.62-3.56 (m, 1H), 3.17 (t, J=10.8 Hz, 1H).

### Preparation of (2R,3R,4R,5S)-2-(hydroxymethyl)-5-((6-methylpyrazin-2-yl)amino)tetrahydro-2H-pyran-3,4-diol (A172)

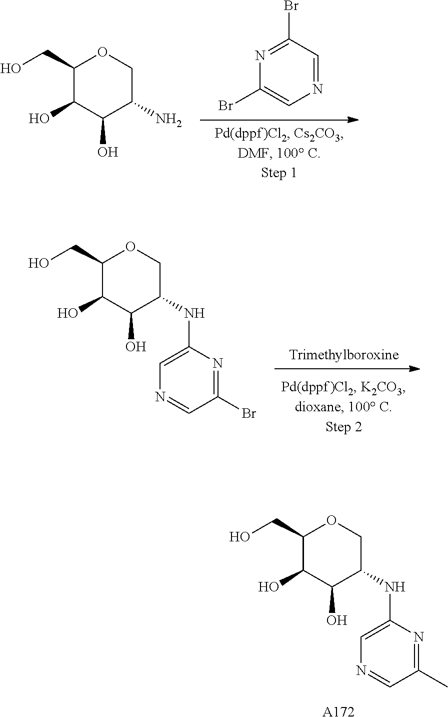 [SMILES: N[C@H]1CO[C@H](CO)[C@H](O)[C@@H]1O ; Brc1cncc(Br)n1 ; OC[C@H]1OC[C@H](Nc2cncc(Br)n2)[C@@H](O)[C@H]1O ; Cc1cncc(N[C@H]2CO[C@H](CO)[C@H](O)[C@@H]2O)n1 ; "A172"]

Step 1: To a solution of (2R,3R,4R,5S)-5-amino-2-(hydroxymethyl)tetrahydro-2H-pyran-3,4-diol (1.00 eq) and 2,6-dibromopyrazine (2.00 eq) in DMF (3 mL) were added Cs₂CO₃ (3.00 eq) and Pd(dppf)C12 (0.1 eq). The mixture was stirred at 100° C. under N₂ for 16 hours, then concentrated. The residue was purified by reverse phase (C18, 5-40% MeOH in water, 0.1% TFA) to afford (2R,3R,4R,5S)-5-((6-bromopyrazin-2-yl)amino)-2-(hydroxymethyl)tetrahydro-2H-pyran-3,4-diol as yellow solid. Yield: 12%. LCMS found: [M+H]⁺=320.
Step 2: To a solution of 3 (1.00 eq) and trimethylboroxine (3.00 eq) in 1,4-dioxane (10 mL) were added K₂CO₃ (2.00 eq) and Pd(dppf)C12 (0.1 eq) at 20° C. The mixture was stirred at 100° C. for 16 hours under nitrogen atmosphere. The mixture was filtered through a Celite pad, and the filtrate was concentrated to give crude product. The residue was purified by reverse phase (C18, 5-35% MeOH in water, 0.1% TFA) to give (2R,3R,4R,5S)-2-(hydroxymethyl)-5-((6-methylpyrazin-2-yl)amino)tetrahydro-2H-pyran-3,4-diol as white solid. Yield: 24%. LCMS found: [M+H]⁺=256. ¹H NMR (400 MHz, D₂O): δ 7.70 (s, 1H), 7.56 (s, 1H), 4.11 (td, J=10.4, 4.8 Hz, 1H), 4.01 (dd, J=11.6, 5.6 Hz, 1H), 3.93 (d, J=3.2 Hz, 1H), 3.70-3.63 (m, 3H), 3.55 (dd, J=7.6, 4.8 Hz, 1H), 3.16 (t, J=11.2 Hz, 1H), 2.26 (s, 3H).

### Preparation of (2R,3R,4R,5S)-2-(((6-aminospiro[3.3]heptan-2-yl)amino)methyl)-5-((6-(trifluoromethyl)pyrazin-2-yl)amino)tetrahydro-2H-pyran-3,4-diol (A173)

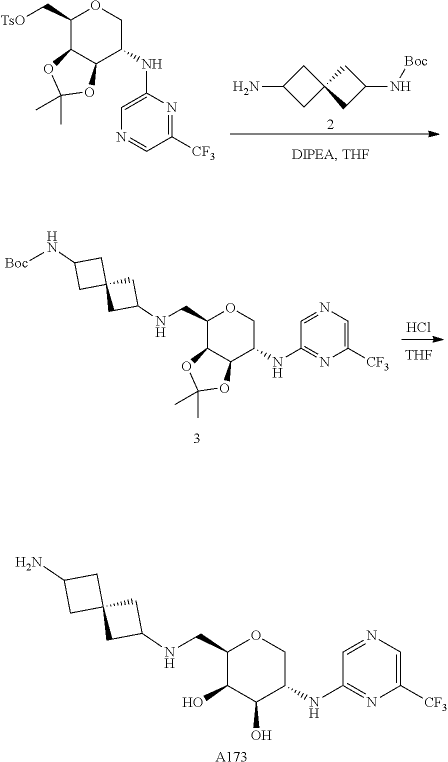

Step 1: To a solution of 1 (1.00 g, 1.00 eq) in THF (5 mL) was added DIEA (1.28 g, 5.00 eq) and 2 (0.45 g, 1.00 eq). The reaction mixture was stirred in sealed pipe at 80° C. for 2 days. LCMS showed the reaction was completed. The mixture was diluted with water, extracted with ethyl acetate, the combined organic layers were washed with saturated solution of sodium bicarbonate, dried over anhydrous sodium sulfate, filtered and concentrated under reduced pressure. The residue was purified by reverse phase (C18, 5-80% MeCN in water, 0.05% TFA) to afford 3 as pale yellow oil. Yield: 72.3%. LCMS found: [M+H]⁺=558.3.
Step 2: To a solution of 3 (0.80 g, 1.00 eq) in THF (10 mL) was added HCl (1 mL, 3 N) at 25° C. The reaction mixture was stirred at 50° C. for 16 hours. LCMS showed the reaction was completed. The mixture was concentrated under reduced pressure and the residue was purified by reverse phase (C18, 5-45% MeCN in water, 0.1% TFA) to afford A173 as a white solid (300 mg, 50.09%). LCMS found: [M+H]⁺=418.2. ¹H NMR (400 MHz, CD₃OD): δ 8.12 (s, 1H), 8.02 (s, 1H), 4.34 (td, J=10.8, 5.2 Hz, 1H), 4.20 (dd, J=10.8, 5.2 Hz, 1H), 3.90 (d, J=2.4 Hz, 1H), 3.77-3.67 (m, 4H), 3.24 (dd, J=12.8, 8.0 Hz, 1H), 3.17 (t, J=10.8 Hz, 1H), 3.13-3.07 (m, 1H), 2.61-2.53 (m, 2H), 2.45-2.37 (m, 2H), 2.34-2.20 (m, 4H).

### Preparation of (2R,3R,4R,5S)-5-((6,7-dihydro-5H-cyclopenta[d]pyrimidin-2-yl)amino)-2-(hydroxymethyl)tetrahydro-2H-pyran-3,4-diol (A174)

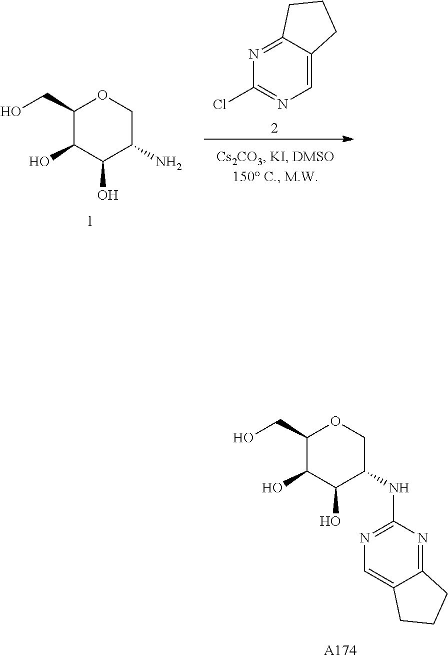 [SMILES: N[C@H]1CO[C@H](CO)[C@H](O)[C@@H]1O ; Clc1ncc2c(n1)CCC2 ; OC[C@H]1OC[C@H](Nc2ncc3c(n2)CCC3)[C@@H](O)[C@H]1O ; "A174"]

To a solution of 1 (1.00 eq) and 2 (2.00 eq) in DMSO (3 mL) was added Cs₂CO₃ (3.00 eq) and KI (0.5 eq), the mixture was stirred at 150° C. for 3 hours in microwave. LCMS showed the desired mass was observed. The mixture was purified by reverse phase (5-40% MeCN in H₂O, 0.1% TFA) to give A174 as a yellow solid. Yield: 5%. LCMS found: [M+H]⁺=282.1. ¹H NMR (400 MHz, CD₃OD): δ 8.27 (s, 1H), 4.45 (d, J=3.6 Hz, 1H), 4.43 (d, J=1.6 Hz, 1H), 4.02-3.95 (m, 2H) 3.88 (t, J=6.0 Hz, 1H), 3.48-3.45 (m, 1H), 3.22 (t, J=10.8 Hz, 1H), 3.14-3.07 (m, 1H), 2.92 (t, J=8.0 Hz, 2H), 2.89 (t, J=8.0 Hz, 2H), 2.20-2.11 (m, 2H).

### Preparation of (2R,3R,4R,5S)-5-((1H-pyrazolo[3,4-d]pyrimidin-6-yl)amino)-2-(hydroxymethyl)tetrahydro-2H-pyran-3,4-diol (A175)

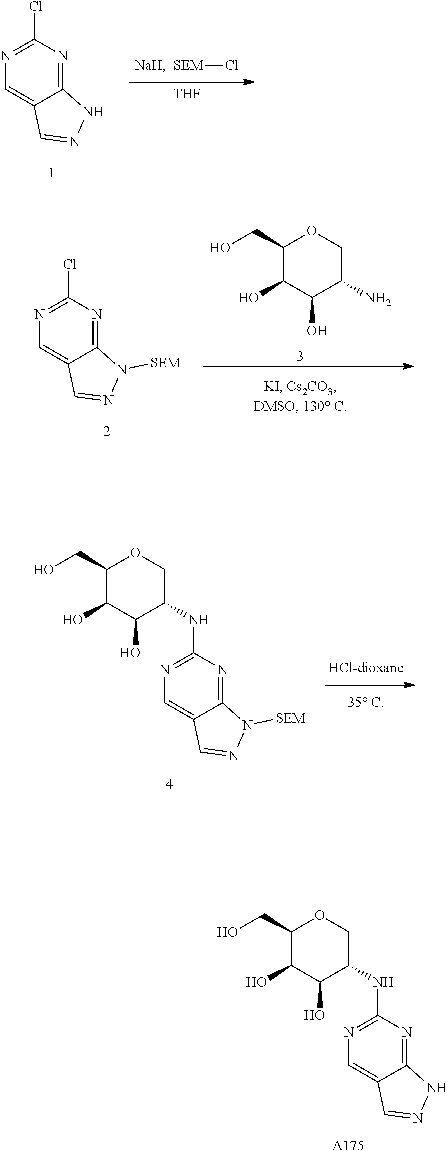

Step 1: To a stirred solution of 1 (1.00 eq) in tetrahydrofuran was added NaH (2.00 eq) at 0° C. and the mixture was stirred for 30 min, then SEM-Cl (3.00 eq) was added. The resulting mixture was stirred at 25° C. for 12 hours. The mixture was quenched with saturated solution of NH₄Cl, extracted with ethyl acetate, the organic layer was dried over anhydrous sodium sulfate, filtered and concentrated under reduced pressure, the residue was purified by silica gel chromatography (PE/EA=10/1 to 1/1) to give 2 as a yellow oil. Yield: 72.1%. LCMS found: [M+H]⁺=285.
Step 2: To a solution of 2 (1.00 eq) and 3 (1.00 eq) in DMSO was added KI (0.2 eq) and Cs₂CO₃ (2.00 eq), the mixture was stirred at 130° C. for 4 hours. The mixture was filtered, the filtrate was purified by reverse phase to afford 4 as a yellow solid. Yield: 24.4%. LCMS found: [M+H]⁺=412.
Step 3: The mixture of 4 (1.00 eq) in HCl/dioxane (4M) was stirred at 35° C. for 4 hours. The mixture was concentrated under reduced pressure, the residue was purified by reverse phase, then further purified by prep-HPLC to give A175 as a white solid. Yield: 14.6%. LCMS found: [M+H]⁺=282. ¹H NMR (400 MHz, CD₃OD) δ 8.77 (s, 1H), 7.92 (s, 1H), 4.45-4.32 (m, 1H), 4.20 (dd, J=10.8, 5.2 Hz, 1H), 3.92 (d, J=3.2 Hz, 1H), 3.80-3.74 (m, 1H), 3.72-3.65 (m, 2H), 3.47-3.43 (m, 1H), 3.18 (t, J=10.8 Hz, 1H).

### Preparation of (2R,3R,4R,5S)-5-((3,3-difluorocyclobutyl)amino)-2-(hydroxymethyl)tetrahydro-2H-pyran-3,4-diol (A176)

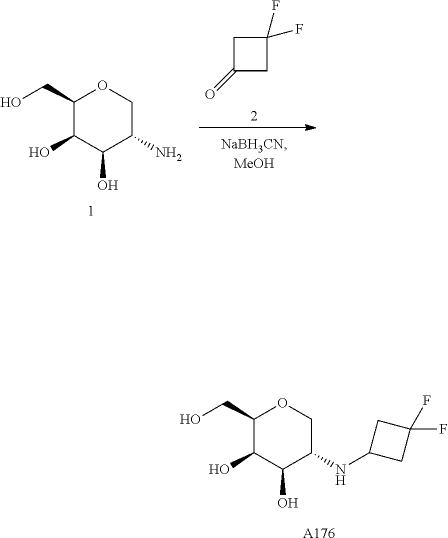

To a solution of 1 (1.00 eq) and 2 (1.30 eq) in MeOH (50 mL) was added NaBH₃CN (3.00 eq) at 0° C. The reaction was stirred at 25° C. for 2 hours. LCMS showed the desired mass was detected. The mixture was purified by prep-HPLC-Mass-guided (0.1% FA) to give A176 as a yellow oil. Yield: 3.5%. LC-MS (ESI) found: 254 [M+H]⁺. ¹H NMR (400 MHz, CD₃OD): δ 4.20 (dd, J=10.8, 4.8 Hz, 1H), 3.94-3.87 (m, 2H), 3.75-3.63 (m, 4H), 3.49-3.42 (m, 2H), 3.12-2.98 (m, 2H), 2.95-2.77 (m, 2H).

### Preparation of (2R,3R,4R,5S)-5-((7H-pyrrolo[2,3-d]pyrimidin-2-yl)amino)-2-(hydroxymethyl)tetrahydro-2H-pyran-3,4-diol (A177)

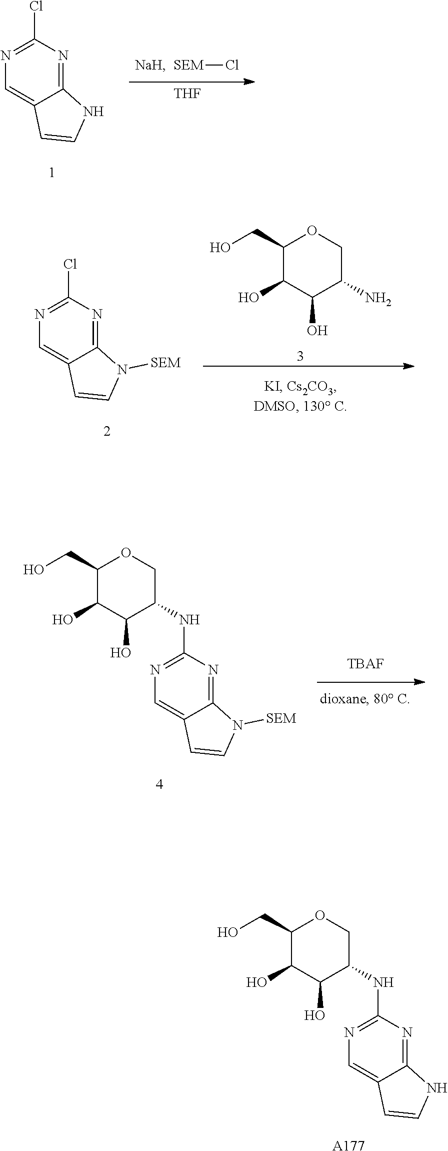

Step 1: To a stirred solution of 1 (1.00 eq) in tetrahydrofuran was added NaH (2.00 eq) at 0° C. and the mixture was stirred for 30 min, then SEM-Cl (3.00 eq) was added. The resulting mixture was stirred at 25° C. for 12 hours. The mixture was quenched with saturated solution of NH₄Cl, extracted with ethyl acetate, the organic layer was dried over anhydrous sodium sulfate, filtered and concentrated under reduced pressure, the residue was purified by silica gel chromatography (PE/EA=10/1 to 1/1) to give 2 as a yellow oil. Yield: 72.0%. LCMS found: [M+H]⁺=284.
Step 2: To a solution of 2 (1.00 eq) and 3 (1.00 eq) in DMSO was added KI (0.20 eq) and Cs₂CO₃ (2.00 eq), the mixture was stirred at 130° C. for 6 hours. The mixture was filtered, the filtrate was purified by reverse phase to afford 4 as a yellow solid. Yield: 19.0%. LCMS found: [M+H]⁺=411.
Step 3: To a solution of 4 (1.00 eq) in dioxane was added TBAF (5.00 eq, 1M in THF), the mixture was stirred at 80° C. for 4 hours. The mixture was concentrated under reduced pressure, the residue was purified by reverse phase (5-40% MeCN in Water, 0.1% TFA), then further purified by prep-HPLC to give A177 as a white solid. Yield: 2.34%. LCMS found: [M+H]⁺=281. ¹H NMR (400 MHz, CD₃OD) δ 8.45 (s, 1H), 6.99 (d, J=3.6 Hz, 1H), 6.34 (d, J=3.6 Hz, 1H), 4.35-4.28 (m, 1H), 4.27-4.21 (m, 1H), 3.91 (d, J=2.4 Hz, 1H), 3.77 (dd, J=11.2, 6.8 Hz, 1H), 3.70 (dd, J=11.2, 5.2 Hz, 1H), 3.63 (dd, J=10.4, 3.2 Hz, 1H), 3.49-3.44 (m, 1H), 3.16 (t, J=10.4 Hz, 1H).

### Preparation of (2R,3R,4R,5S)-5-((6-(difluoromethoxy)pyridin-2-yl)amino)-2-(hydroxymethyl)tetrahydro-2H-pyran-3,4-diol (A178)

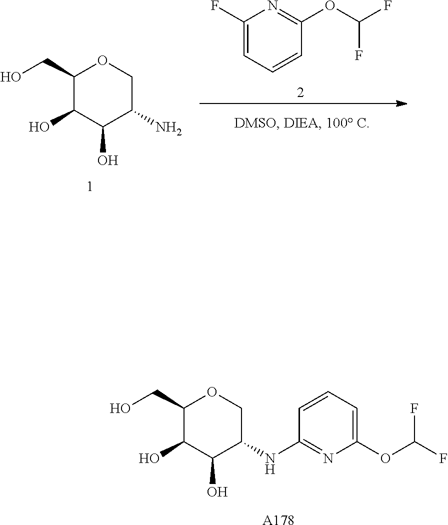 [SMILES: N[C@H]1CO[C@H](CO)[C@H](O)[C@@H]1O ; Fc1cccc(OC(F)F)n1 ; OC[C@H]1OC[C@H](Nc2cccc(OC(F)F)n2)[C@@H](O)[C@H]1O ; "A178"]

To a solution of 1 (1.00 eq) and 2 (1.10 eq) in DMSO (1 mL) was added DIEA (5.0 eq) dropwise at room temperature. The reaction was stirred at 100° C. for 48 hours. LCMS showed the desired mass was detected. The crude product was purified by prep-HPLC to give A178 as brown solid. Yield: 5.33%. LCMS found: [M+H]⁺=307. ¹H NMR (400 MHz, CD₃OD): δ 7.57 (t, J=74.0 Hz, 1H), 7.40 (t, J=8.0 Hz, 1H), 6.28 (d, J=8.0 Hz, 1H), 6.03 (d, J=8.0 Hz, 1H), 4.21 (td, J=10.4, 5.2 Hz, 1H), 4.12 (dd, J=10.8, 5.2 Hz, 1H), 3.90 (d, J=2.8 Hz, 1H), 3.80-3.65 (m, 2H), 3.57 (dd, J=10.4, 3.2 Hz, 1H), 3.47-3.41 (m, 1H), 3.06 (t, J=10.8 Hz, 1H).

### Preparation of (2S,3R,4R,5S)-2-(difluoromethyl)-5-((6-(trifluoromethyl)pyrazin-2-yl)amino)tetrahydro-2H-pyran-3,4-diol (A179)

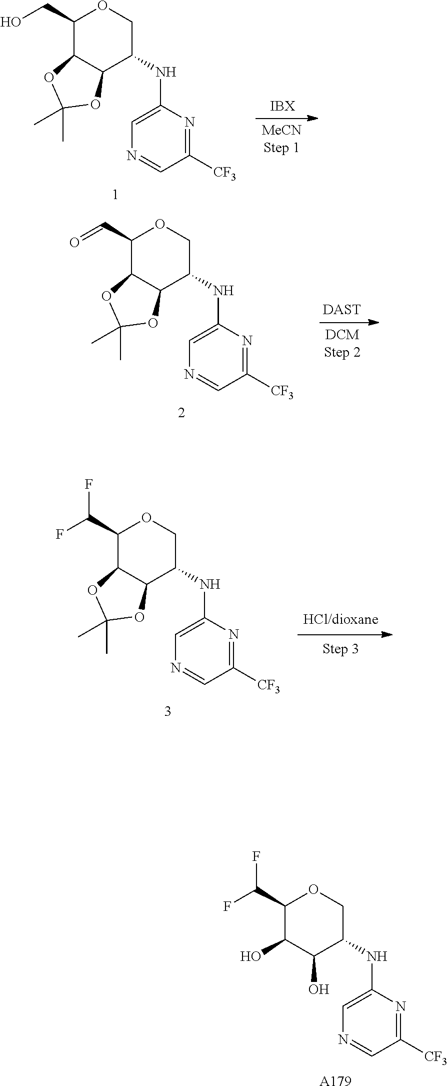

Step 1: To a solution of 1 (1.00 eq) in MeCN (2 mL) was added IBX (2.00 eq) at 25° C. The reaction was stirred at 80° C. for 1 hour. LCMS showed the desired mass was detected. The resulting mixture was filtered and concentrated in vacuo to give crude 3 as a colorless oil. LC-MS (ESI) found: 348 [M+H]⁺.
Step 2: To a solution of 2 (1.00 eq) in DCM (2 mL) was added DAST (5.00 eq) dropwise at −78° C. The reaction was stirred at 25° C. for 12 hours. LCMS showed the desired mass was detected. Then the mixture was cooled down to −30° C. and quenched with MeOH (1 mL). The mixture was concentrated and the residue was dissolved in DCM (10 mL), washed with saturated aqueous NaHCO₃ and brine, dried with anhydrous Na₂SO₄. The organic layer was separated and concentrated in vacuo. The crude product was purified by flash chromatography (silica gel, 0˜60% EA in PE) to give 3 as a yellow oil. Yield: 22.6%. LC-MS (ESI) found: 370 [M+H]⁺.
Step 3: The solution of 3 (1.00 eq) in HCl/1,4-dioxane (1 mL, 4 N) was stirred at 25° C. for 1 hour. LCMS showed the desired mass was detected. The mixture was concentrated in vacuo and the residue was purified by prep-HPLC to give A179 as a white solid. Yield: 10.1%. LCMS found: [M+H]⁺=330. ¹H NMR (400 MHz, CD₃OD): δ 8.12 (s, 1H), 8.02 (s, 1H), 5.91 (ddd, J=57.2, 54.8, 6.4 Hz, 1H), 4.37 (td, J=10.4, 4.8 Hz, 1H), 4.18 (dd, J=11.2, 5.2 Hz, 1H), 4.05-4.03 (m, 1H), 3.68 (dd, J=10.6, 3.2 Hz, 1H), 3.62-3.56 (m, 1H), 3.17 (t, J=10.8 Hz, 1H).

### Preparation of (2R,3R,4R,5S)-2-(hydroxymethyl)-5-((6-methylpyrazin-2-yl)amino)tetrahydro-2H-pyran-3,4-diol (A180)

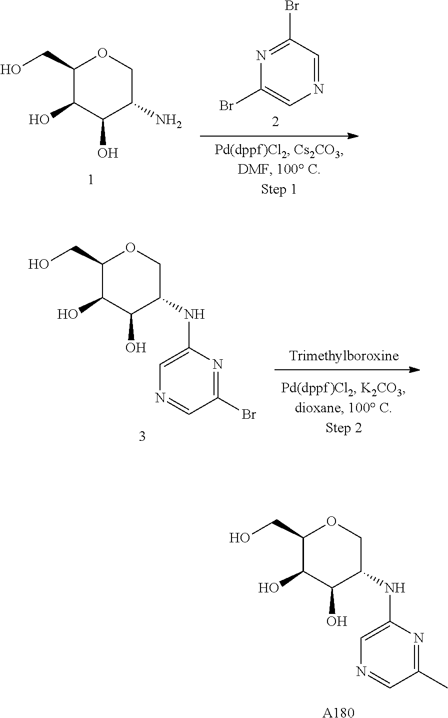 [SMILES: N[C@H]1CO[C@H](CO)[C@H](O)[C@@H]1O ; Brc1cncc(Br)n1 ; OC[C@H]1OC[C@H](Nc2cncc(Br)n2)[C@@H](O)[C@H]1O ; Cc1cncc(N[C@H]2CO[C@H](CO)[C@H](O)[C@@H]2O)n1 ; "A180"]

Step 1: To a solution of 1 (1.00 eq) and 2 (2.00 eq) in DMF (3 mL) were added Cs₂CO₃ (3.00 eq) and Pd(dppf)Cl₂ (0.1 eq). The mixture was stirred at 100° C. under N₂ for 16 hours, then concentrated. The residue was purified by reverse phase (C18, 5-40% MeOH in water, 0.1% TFA) to afford 3 as yellow solid. Yield: 12%. LCMS found: [M+H]⁺=320.
Step 2: To a solution of 3 (1.00 eq) and trimethylboroxine (3.00 eq) in 1,4-dioxane (10 mL) were added K₂CO₃ (2.00 eq) and Pd(dppf)C12 (0.1 eq) at 20° C. The mixture was stirred at 100° C. for 16 hours under nitrogen atmosphere. The mixture was filtered through a Celite pad, and the filtrate was concentrated to give crude product. The residue was purified by reverse phase (C18, 5-35% MeOH in water, 0.1% TFA) to give A180 as white solid. Yield: 24%. LCMS found: [M+H]⁺=256. ¹H NMR (400 MHz, D₂O): δ 7.70 (s, 1H), 7.56 (s, 1H), 4.11 (td, J=10.4, 4.8 Hz, 1H), 4.01 (dd, J=11.6, 5.6 Hz, 1H), 3.93 (d, J=3.2 Hz, 1H), 3.70-3.63 (m, 3H), 3.55 (dd, J=7.6, 4.8 Hz, 1H), 3.16 (t, J=11.2 Hz, 1H), 2.26 (s, 3H).

### Preparation of (2R,3R,4R,5S)-2-(((6-aminospiro[3.3]heptan-2-yl)amino)methyl)-5-((6-(trifluoromethyl)pyrazin-2-yl)amino)tetrahydro-2H-pyran-3,4-diol (A181)

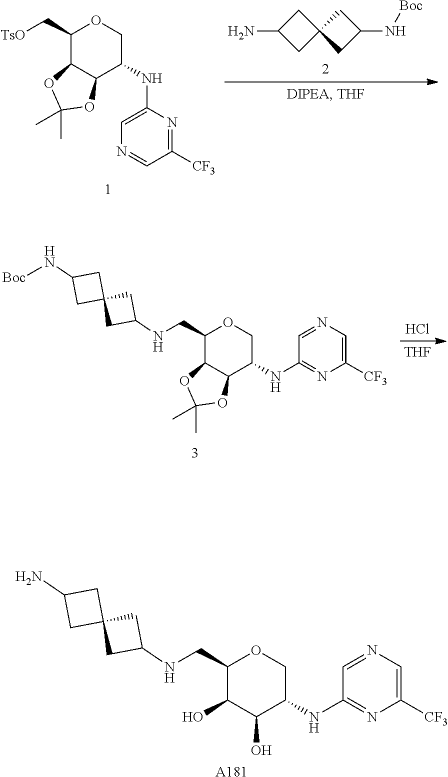

Step 1: To a solution of 1 (1.00 g, 1.00 eq) in THF (5 mL) was added DIEA (1.28 g, 5.00 eq) and 2 (0.45 g, 1.00 eq). The reaction mixture was stirred in sealed pipe at 80° C. for 2 days. LCMS showed the reaction was completed. The mixture was diluted with water, extracted with ethyl acetate, the combined organic layers were washed with saturated solution of sodium bicarbonate, dried over anhydrous sodium sulfate, filtered and concentrated under reduced pressure. The residue was purified by reverse phase (C18, 5-80% MeCN in water, 0.05% TFA) to afford 3 as pale yellow oil. Yield: 72.3%. LCMS found: [M+H]⁺=558.3.
Step 2: To a solution of 3 (0.80 g, 1.00 eq) in THF (10 mL) was added HCl (1 mL, 3 N) at 25° C. The reaction mixture was stirred at 50° C. for 16 hours. LCMS showed the reaction was completed. The mixture was concentrated under reduced pressure and the residue was purified by reverse phase (C18, 5-45% MeCN in water, 0.1% TFA) to afford A181 as a white solid (300 mg, 50.09%). LCMS found: [M+H]⁺=418.2. ¹H NMR (400 MHz, CD₃OD): δ 8.12 (s, 1H), 8.02 (s, 1H), 4.34 (td, J=10.8, 5.2 Hz, 1H), 4.20 (dd, J=10.8, 5.2 Hz, 1H), 3.90 (d, J=2.4 Hz, 1H), 3.77-3.67 (m, 4H), 3.24 (dd, J=12.8, 8.0 Hz, 1H), 3.17 (t, J=10.8 Hz, 1H), 3.13-3.07 (m, 1H), 2.61-2.53 (m, 2H), 2.45-2.37 (m, 2H), 2.34-2.20 (m, 4H).

### Preparation of (2R,3R,4R,5S)-5-((6,7-dihydro-5H-cyclopenta[d]pyrimidin-2-yl)amino)-2-(hydroxymethyl)tetrahydro-2H-pyran-3,4-diol (A182)

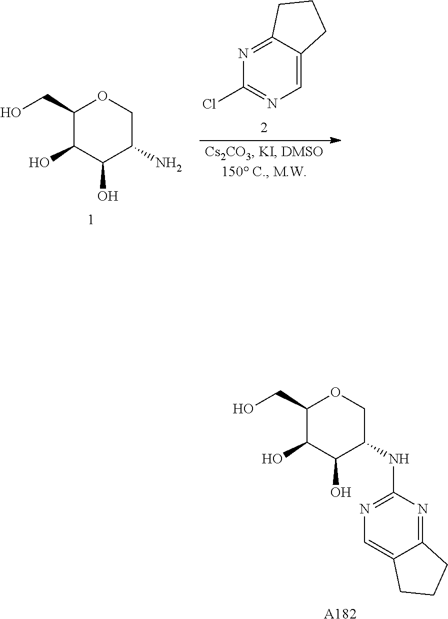 [SMILES: N[C@H]1CO[C@H](CO)[C@H](O)[C@@H]1O ; Clc1ncc2c(n1)CCC2 ; OC[C@H]1OC[C@H](Nc2ncc3c(n2)CCC3)[C@@H](O)[C@H]1O ; "A182"]

Step 1: To a solution of 1 (1.00 eq) and 2 (2.00 eq) in DMSO (3 mL) was added Cs₂CO₃ (3.00 eq) and KI (0.5 eq), the mixture was stirred at 150° C. for 3 hours in microwave. LCMS showed the desired mass was observed. The mixture was purified by reverse phase (5-40% MeCN in H₂O, 0.1% TFA) to give A182 as a yellow solid. Yield: 5%. LCMS found: [M+H]⁺=282.1. ¹H NMR (400 MHz, CD₃OD): δ 8.27 (s, 1H), 4.45 (d, J=3.6 Hz, 1H), 4.43 (d, J=1.6 Hz, 1H), 4.02-3.95 (m, 2H), 3.88 (t, J=6.0 Hz, 1H), 3.48-3.45 (m, 1H), 3.22 (t, J=10.8 Hz, 1H), 3.14-3.07 (m, 1H), 2.92 (t, J=8.0 Hz, 2H), 2.89 (t, J=8.0 Hz, 2H), 2.20-2.11 (m, 2H).

### Preparation of (2R,3R,4R,5S)-5-((1H-pyrazolo[3,4-d]pyrimidin-6-yl)amino)-2-(hydroxymethyl)tetrahydro-2H-pyran-3,4-diol (A183)

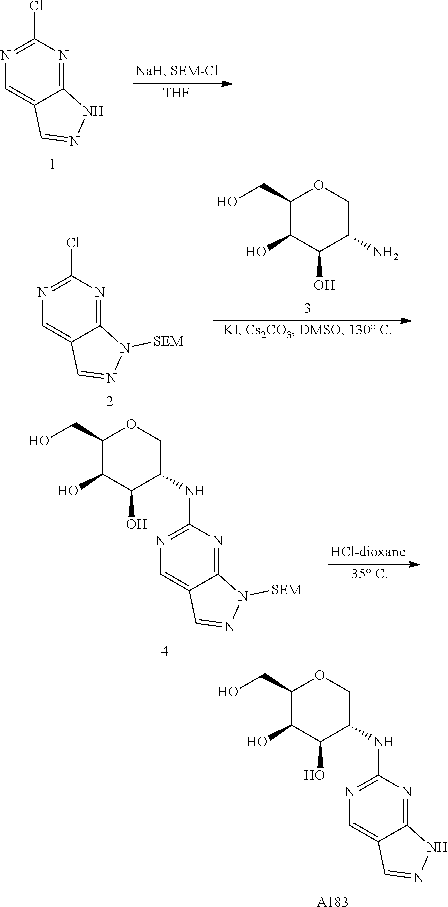

Step 1: To a stirred solution of 1 (1.00 eq) in tetrahydrofuran was added NaH (2.00 eq) at 0° C. and the mixture was stirred for 30 min, then SEM-Cl (3.00 eq) was added. The resulting mixture was stirred at 25° C. for 12 hours. The mixture was quenched with saturated solution of NH₄Cl, extracted with ethyl acetate, the organic layer was dried over anhydrous sodium sulfate, filtered and concentrated under reduced pressure, the residue was purified by silica gel chromatography (PE/EA=10/1 to 1/1) to give 2 as a yellow oil. Yield: 72.1%. LCMS found: [M+H]⁺=285.
Step 2: To a solution of 2 (1.00 eq) and 3 (1.00 eq) in DMSO was added KI (0.2 eq) and Cs₂CO₃ (2.00 eq), the mixture was stirred at 130° C. for 4 hours. The mixture was filtered, the filtrate was purified by reverse phase to afford 4 as a yellow solid. Yield: 24.4%. LCMS found: [M+H]⁺=412.
Step 3: The mixture of 4 (1.00 eq) in HCl/dioxane (4M) was stirred at 35° C. for 4 hours. The mixture was concentrated under reduced pressure, the residue was purified by reverse phase, then further purified by prep-HPLC to give A183 as a white solid. Yield: 14.6%. LCMS found: [M+H]⁺=282. ¹H NMR (400 MHz, CD₃OD) δ 8.77 (s, 1H), 7.92 (s, 1H), 4.45-4.32 (m, 1H), 4.20 (dd, J=10.8, 5.2 Hz, 1H), 3.92 (d, J=3.2 Hz, 1H), 3.80-3.74 (m, 1H), 3.72-3.65 (m, 2H), 3.47-3.43 (m, 1H), 3.18 (t, J=10.8 Hz, 1H).

### Preparation of (2R,3R,4R,5S)-5-((7H-pyrrolo[2,3-d]pyrimidin-2-yl)amino)-2-(hydroxymethyl)tetrahydro-2H-pyran-3,4-diol (A184)

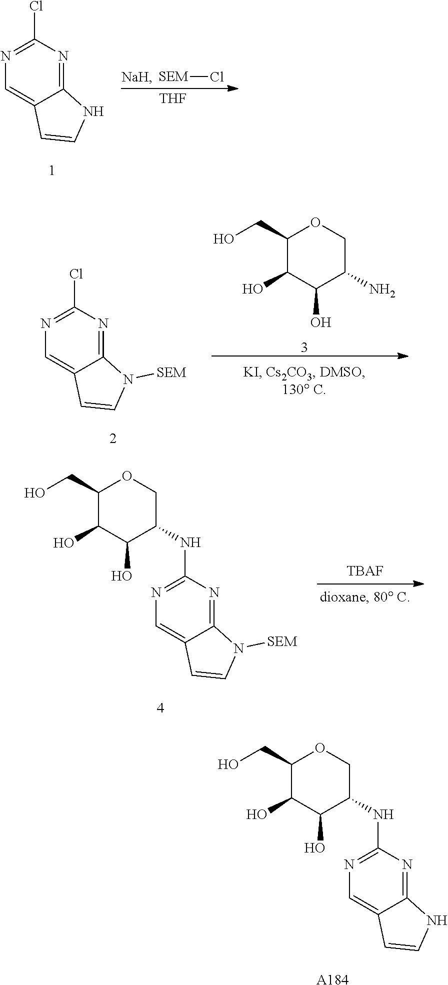

Step 1: To a stirred solution of 1 (1.00 eq) in tetrahydrofuran was added NaH (2.00 eq) at 0° C. and the mixture was stirred for 30 min, then SEM-Cl (3.00 eq) was added. The resulting mixture was stirred at 25° C. for 12 hours. The mixture was quenched with saturated solution of NH₄Cl, extracted with ethyl acetate, the organic layer was dried over anhydrous sodium sulfate, filtered and concentrated under reduced pressure, the residue was purified by silica gel chromatography (PE/EA=10/1 to 1/1) to give 2 as a yellow oil. Yield: 72.0%. LCMS found: [M+H]⁺=284.
Step 2: To a solution of 2 (1.00 eq) and 3 (1.00 eq) in DMSO was added KI (0.20 eq) and Cs₂CO₃ (2.00 eq), the mixture was stirred at 130° C. for 6 hours. The mixture was filtered, the filtrate was purified by reverse phase to afford 4 as a yellow solid. Yield: 19.0%. LCMS found: [M+H]⁺=411.
Step 3: To a solution of 4 (1.00 eq) in dioxane was added TBAF (5.00 eq, 1M in THF), the mixture was stirred at 80° C. for 4 hours. The mixture was concentrated under reduced pressure, the residue was purified by reverse phase (5-40% MeCN in Water, 0.1% TFA), then further purified by prep-HPLC to give A184 as a white solid. Yield: 2.34%. LCMS found: [M+H]⁺=281. ¹H NMR (400 MHz, CD₃OD) δ 8.45 (s, 1H), 6.99 (d, J=3.6 Hz, 1H), 6.34 (d, J=3.6 Hz, 1H), 4.35-4.28 (m, 1H), 4.27-4.21 (m, 1H), 3.91 (d, J=2.4 Hz, 1H), 3.77 (dd, J=11.2, 6.8 Hz, 1H), 3.70 (dd, J=11.2, 5.2 Hz, 1H), 3.63 (dd, J=10.4, 3.2 Hz, 1H), 3.49-3.44 (m, 1H), 3.16 (t, J=10.4 Hz, 1H).

### Preparation of (2R,3R,4R,5S)-5-((6-(difluoromethoxy)pyridin-2-yl)amino)-2-(hydroxymethyl)tetrahydro-2H-pyran-3,4-diol (A185)

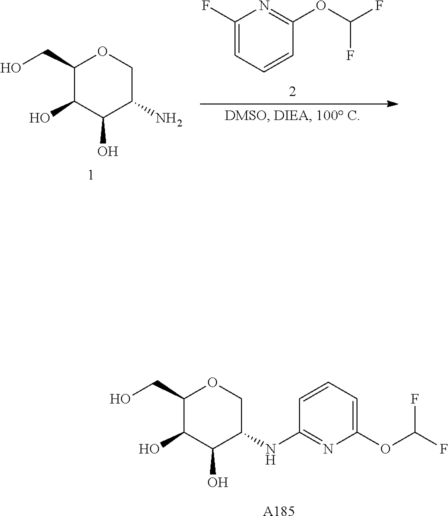 [SMILES: N[C@H]1CO[C@H](CO)[C@H](O)[C@@H]1O ; Fc1cccc(OC(F)F)n1 ; OC[C@H]1OC[C@H](Nc2cccc(OC(F)F)n2)[C@@H](O)[C@H]1O ; "A185"]

Step 1: To a solution of 1 (1.00 eq) and 2 (1.10 eq) in DMSO (1 mL) was added DIEA (5.0 eq) dropwise at room temperature. The reaction was stirred at 100° C. for 48 hours. LCMS showed the desired mass was detected. The crude product was purified by prep-HPLC to give A185 as brown solid. Yield: 5.33%. LCMS found: [M+H]⁺=307. ¹H NMR (400 MHz, CD₃OD): δ 7.57 (t, J=74.0 Hz, 1H), 7.40 (t, J=8.0 Hz, 1H), 6.28 (d, J=8.0 Hz, 1H), 6.03 (d, J=8.0 Hz, 1H), 4.21 (td, J=10.4, 5.2 Hz, 1H), 4.12 (dd, J=10.8, 5.2 Hz, 1H), 3.90 (d, J=2.8 Hz, 1H), 3.80-3.65 (m, 2H), 3.57 (dd, J=10.4, 3.2 Hz, 1H), 3.47-3.41 (m, 1H), 3.06 (t, J=10.8 Hz, 1H).

### Preparation of 6-(((2R,3R,4R,5S)-3,4-dihydroxy-5-((6-(trifluoromethyl)pyrazin-2-yl) amino)tetrahydro-2H-pyran-2-yl)methoxy)pyridazine-3-carboxylic acid (A186)

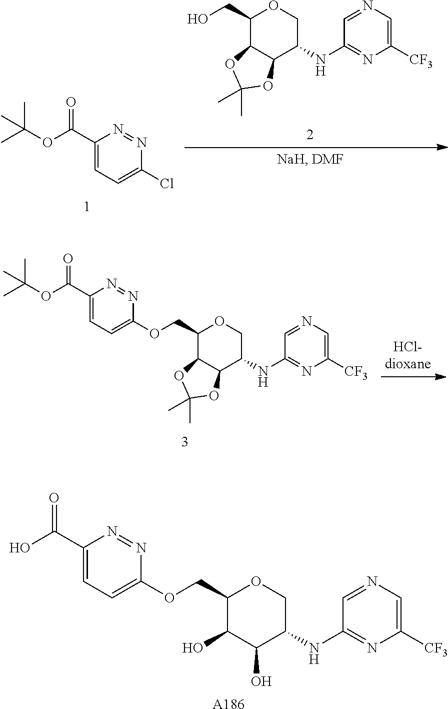 [SMILES: CC(C)(C)OC(=O)c1ccc(Cl)nn1 ; CC1(C)O[C@@H]2[C@H](O1)[C@@H](Nc1cncc(C(F)(F)F)n1)CO[C@@H]2CO ; CC(C)(C)OC(=O)c1ccc(OC[C@H]2OC[C@H](Nc3cncc(C(F)(F)F)n3)[C@H]3OC(C)(C)O[C@H]32)nn1 ; O=C(O)c1ccc(OC[C@H]2OC[C@H](Nc3cncc(C(F)(F)F)n3)[C@@H](O)[C@H]2O)nn1 ; "A186"]

Step 1: To a solution of 2 (1.00 eq) in DMF was added NaH (1.20 eq, 60% purity) at 0° C., the mixture was stirred at 0° C. for 20 min, then 1 (1.20 eq) was added to the above mixture at 0° C. and the resulting mixture was stirred at 20° C. overnight. The mixture was quenched with saturated solution of NH₄Cl, extracted with ethyl acetate, the organic layer was dried over anhydrous sodium sulfate, filtered and concentrated under reduced pressure, the residue was purified by silica gel chromatography (DCM:MeOH=15:1) to afford 3 as a yellow solid. Yield: 26.8%. LCMS found: [M+H]⁺=528.
Step 2: The solution of 3 (1.00 eq) in HCl/dioxane (4M) was stirred at 50° C. for 3 hours. The mixture was concentrated and purified by reverse phase (5-50% MeCN in water, 0.1% FA) to give A186 as a yellow solid. Yield: 58.1%. LCMS found: [M+H]⁺=432. ¹H NMR (400 MHz, CD₃OD) δ 8.19 (d, J=9.2 Hz, 1H), 8.12 (s, 1H), 8.01 (s, 1H), 7.31 (d, J=9.2 Hz, 1H), 4.79-4.68 (m, 2H) 4.38 (td, J=10.8, 5.2 Hz, 1H), 4.16 (dd, J=11.2, 5.2 Hz, 1H), 4.04 (d, J=2.8 Hz, 1H), 3.97-3.90 (m, 1H), 3.71 (dd, J=10.8, 3.2 Hz, 1H), 3.17 (t, J=10.8 Hz, 1H).

### Preparation of 5-(((2R,3R,4R,5S)-3,4-dihydroxy-5-((6-(trifluoromethyl)pyrazin-2-yl)amino)tetrahydro-2H-pyran-2-yl)methoxy)pyrazine-2-carboxylic acid (A187)

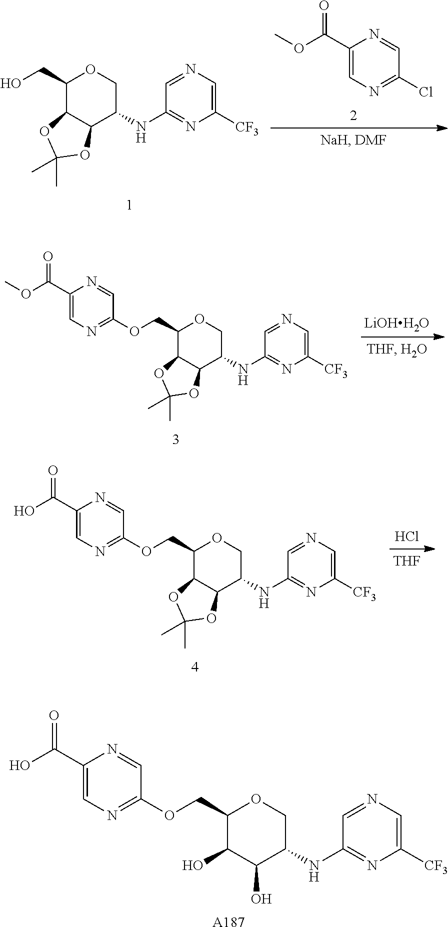

Step 1: To a solution of 1 (1.00 eq) in DMF (4 mL) was added Sodium Hydride (2.00 eq, 60% purity) at 0° C., the mixture was stirred at 0° C. for 30 minutes, then 2 (2.00 eq) was added, the resulting mixture was stirred at room temperature for 3 hours. LCMS showed the reaction was completed, the reaction mixture was quenched with saturated solution of NH₄Cl, then extracted with EtOAc, the organic layer was washed with brine and water, dried over anhydrous sodium sulfate, filtered and concentrated under reduced pressure, the residue was purified by silica gel chromatography (DCM/MeOH=20/1) to afford 3 as yellow solid. Yield: 65%. Chemical Formula: C₂₀H₂₂F₃N₅O₆, LCMS found: [M+H]⁺=486.
Step 2: To a solution of 3 (1.00 eq) in THF (5 mL) and H₂O (1 mL) was added Lithium Hydroxide Monohydrate (5.00 eq), the mixture was stirred at room temperature for 12 hours. LCMS showed the reaction was completed. The mixture was concentrated under reduced pressure to give 4 (crude) as a white solid. Chemical Formula: C₁₉H₂₀F₃N₅O₆, LCMS found: [M+H]⁺=472.
Step 3: To a solution of 4 (1.00 eq) in THF (5 mL) was added HCl (1 mL, 2N), the mixture was stirred at 50° C. for 12 hours. LCMS showed the reaction was completed. The mixture was concentrated under reduced pressure. The residue was purified by reverse phase (5-26% MeCN in water, 0.1% FA) to afford A187 as a white solid. Yield: 32.3%. Chemical Formula: C₁₆H₁₆F₃N₅O₆, LCMS found: [M+H]⁺=432. ¹H NMR (400 MHz, CD₃OD): δ 8.88 (s, 1H), 8.29 (s, 1H), 8.12 (s, 1H), 8.01 (s, 1H), 4.69-4.59 (m, 2H), 4.43-4.34 (m, 1H), 4.16 (dd, J=10.9, 4.9 Hz, 1H), 4.03 (d, J=2.4 Hz, 1H), 3.90 (t, J=5.6 Hz, 1H), 3.71 (dd, J=10.5, 2.9 Hz, 1H) 3.18 (t, J=10.9 Hz, 1H).

### Preparation of 6-(((2R,3R,4R,5S)-3,4-dihydroxy-5-((6-(trifluoromethyl)pyrazin-2-yl)amino)tetrahydro-2H-pyran-2-yl)methoxy)pyridazine-3-carbonitrile (A188)

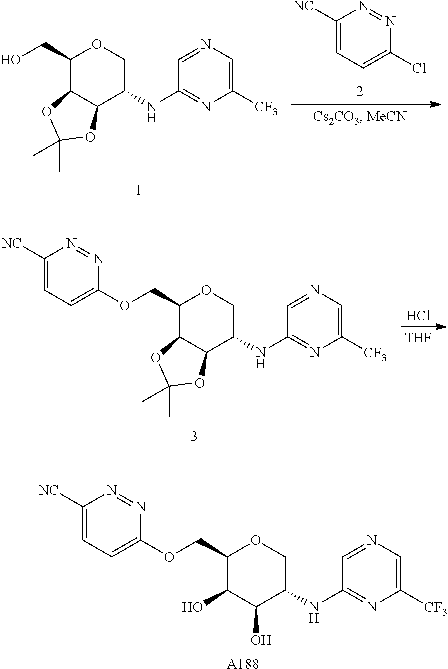

Step 1: To a solution of 1 (1.00 eq), 2 (1.50 eq) and Cs₂CO₃ (2.00 eq) in CH₃CN was stirred at 50° C. for 12 hours. The mixture was concentrated under reduced pressure, the residue was purified by Flash (PE:EA=3:1 to 1:4) to give 3 as a yellow solid. Yield: 35.8%. LC-MS (ESI) found: 453 [M+H]⁺.
Step 2: To a solution of 3 (1.00 eq) in THE was added HCl (1.2 M, 10.00 eq), the mixture was stirred at 25° C. for 12 hours. The mixture was concentrated under reduced pressure, the residue was purified by Prep-HPLC to give A188 as a white solid. Yield: 43.2%. LCMS found: 413 [M+H]⁺. ¹H NMR (400 MHz, CD₃OD) δ 8.12 (s, 1H), 8.01 (s, 1H), 7.98 (d, J=9.4 Hz, 1H), 7.34 (d, J=9.4 Hz, 1H), 4.80-4.74 (m, 2H), 4.38 (td, J=10.8, 5.4 Hz, 1H), 4.16 (dd, J=11.2, 5.4 Hz, 1H), 4.05-4.03 (m, 1H), 3.97-3.91 (m, 1H), 3.71 (dd, J=10.8, 3.2 Hz, 1H), 3.18 (t, J=10.8 Hz, 1H). ¹⁹F NMR (377 MHz, CD₃OD) δ −70.33 (s).

### Preparation of (2R,3R,4R,5S)-2-(hydroxymethyl)-5-((6-(prop-1-yn-1-yl)pyridin-2-yl)amino)tetrahydro-2H-pyran-3,4-diol (A189)

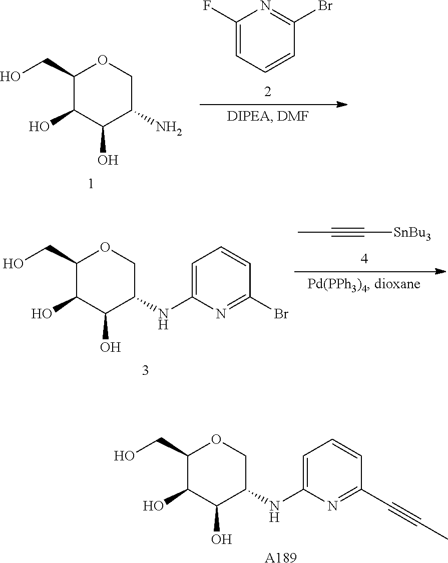

Step 1: To a solution of 1 (1.00 eq) and 2 (2.00 eq) in DMF (5 mL) was added DIEA (3.00 eq), the mixture was stirred at 100° C. for 16 hours. LCMS showed the desired mass was observed. The mixture was concentrated under reduced pressure, the residue was purified by reverse phase (5-30% MeOH in water, 0.1% TFA) to give compound 3 as a yellow solid. Yield: 17%. LCMS found: [M+H]⁺=319.2, 321.2.
Step 2: To a solution of 3 (1.00 eq) and 4 (3.00 eq) in dioxane (5 mL) was added Pd(PPh₃)₄ (0.10 eq), the mixture was degassed and purged with nitrogen for 3 times, then stirred at 100° C. for 16 hours under nitrogen atmosphere. LCMS showed the desired mass was detected. The mixture was quenched with saturated solution of KF, then concentrated under reduced pressure. The residue was purified by reverse phase (5-35% MeOH in water, 0.1% TFA) to give A189 as a yellow solid. Yield: 22%. LCMS found: [M+H]⁺=279.1. ¹H NMR (400 MHz, CD₃OD): δ ¹H NMR (400 MHz, D20) 6 7.41 (dd, J=8.4, 7.6 Hz, 1H), 6.72 (d, J=7.6 Hz, 1H), 6.53 (d, J=8.4 Hz, 1H), 4.03-3.96 (m, 2H), 3.92 (d, J=3.2 Hz, 1H), 3.73-3.60 (m, 3H), 3.53 (dd, J=8.0, 4.0 Hz, 1H), 3.12 (t, J=12.0 Hz, 1H), 1.96 (s, 3H).

### Preparation of (2R,3R,4S,5S)-2-(([1,1′-biphenyl]-4-yloxy)methyl)-5-((6-(trifluoromethyl)pyridin-2-yl)oxy)tetrahydro-2H-pyran-3,4-diol (A190)

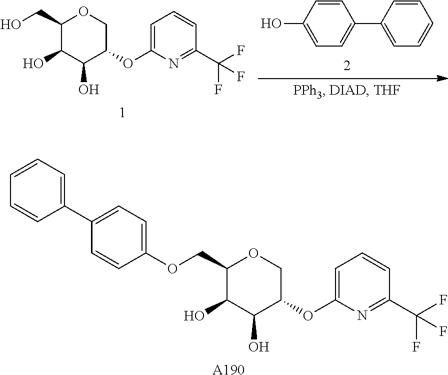 [SMILES: OC[C@H]1OC[C@H](Oc2cccc(C(F)(F)F)n2)[C@@H](O)[C@H]1O ; Oc1ccc(-c2ccccc2)cc1 ; O[C@@H]1[C@H](O)[C@@H](Oc2cccc(C(F)(F)F)n2)CO[C@@H]1COc1ccc(-c2ccccc2)cc1 ; "A190"]

Step 1: To a solution of 1 (1.00 eq), 2 (1.20 eq) and PPh₃ (4.00 eq) in THF (3 mL) was added DIAD (4.00 eq) dropwise at 0° C. under nitrogen atmosphere. The mixture was stirred at 60° C. for 3 hours under nitrogen atmosphere. The desired mass was observed by LCMS. The mixture was concentrated under reduced pressure. The residue was purified by reverse phase (C18, 5-70% MeCN in water, 0.1% TFA) to afford A190 as white solid. Yield: 4.1%. LCMS found: [M+H]⁺=462. ¹H NMR (400 MHz, CD₃OD): δ 7.87 (t, J=7.9 Hz, 1H), 7.65-7.51 (m, 4H), 7.43-7.34 (m, 3H), 7.28 (d, J=7.4 Hz, 1H), 7.07-7.02 (m, 3H), 5.41 (td, J=10.0, 5.6 Hz, 1H), 4.39 (dd, J=10.8, 5.4 Hz, 1H), 4.29-4.15 (m, 2H), 4.12 (d, J=3.0 Hz, 1H), 3.93-3.80 (m, 2H), 3.34 (t, J=10.8 Hz, 1H).

### Preparation of (2R,3R,4R,5S)-2-ethynyl-5-((6-(trifluoromethyl)pyrazin-2-yl)amino)tetrahydro-2H-pyran-3,4-diol (A191)

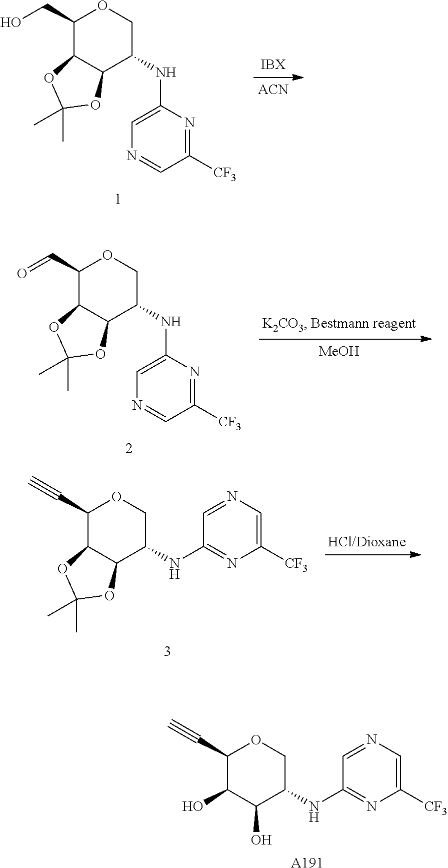

Step 1: To a solution of 1 (1.00 eq) in CH₃CN (2 mL) was added IBX (2.00 eq) at 25° C. The reaction was stirred at 80° C. for 1 hour. LCMS showed the desired mass was detected. The mixture was filtered and the filtrate was concentrated in vacuo to give crude 2 as a colorless oil. LC-MS (ESI) found: 348 [M+H]⁺.
Step 2: To a solution of 2 (1.00 eq) and K₂CO₃ (2.00 eq) in MeOH (5 mL) was added Bestmann-Ohira reagent (1.20 eq) dropwise at 0° C. The reaction was stirred at 25° C. for 3 hours. LCMS showed the desired mass was detected. The mixture was concentrated under reduced pressure, the residue was diluted with water, then extracted with EtOAc, the organic layer was washed with saturated solution of NaHCO₃ and brine, dried with anhydrous Na₂SO₄, filtered and concentrated under reduced pressure. The crude product was purified by flash chromatography (silica gel, 0˜70% EA in PE) to give 3 as a yellow oil. Yield: 32.9%. LC-MS (ESI) found: 344 [M+H]⁺.
Step 3: The solution of 3 (1.00 eq) in HCl/1,4-dioxane (1 mL) was stirred at 25° C. for 1 hour. LCMS showed the desired mass was detected. The mixture was concentrated in vacuo. The crude product was purified by prep-HPLC to give A191 as a white solid. Yield: 21.5%. LCMS found: [M+H]⁺=304. ¹H NMR (400 MHz, CD₃OD): δ 8.12 (s, 1H), 8.01 (s, 1H), 4.35-4.28 (m, 2H) 4.11 (dd, J=11.2, 5.2 Hz, 1H), 3.93 (d, J=1.6 Hz, 1H), 3.68 (dd, J=10.2, 3.2 Hz, 1H), 3.14 (t, J=10.8 Hz, 1H), 2.90 (d, J=2.4 Hz, 1H).

### Preparation of (2R,3R,4S,5S)-2-(aminomethyl)-5-((6-(trifluoromethyl)pyridin-2-yl)oxy)tetrahydro-2H-pyran-3,4-diol (A192)

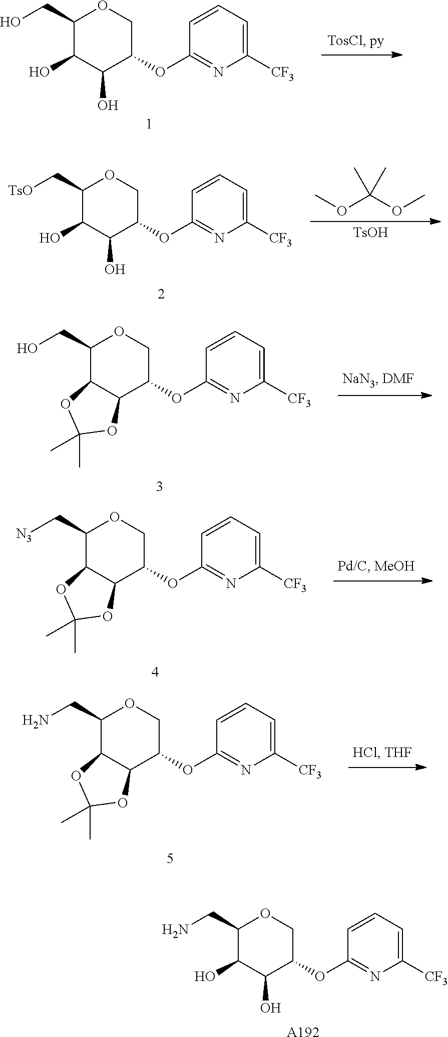

Step 1: A solution of 1 (1.00 eq) and TosCl (1.50 eq) in pyridine (3 mL) was stirred at 40° C. for 3 hours. The mixture was concentrated under reduced pressure. The residue was purified by reverse phase (C18, 5-85% MeCN in water, 0.1% TFA) to afford 2 as brown oil. Yield: 60%. LCMS found: [M+H]⁺=464.
Step 2: A solution of 2 (1.00 eq) and TsOH (0.10 eq) in 2,2-dimethoxypropane (3 mL) was stirred at 80° C. for 10 hours. LCMS showed the desired mass was detected. The mixture was quenched with Et₃N (1 mL) and then concentrated. The residue was purified by silica gel chromatography (PE/EA=20/1 to 8/1) to afford 3 as colorless oil. Yield: 71.6%. LCMS found: [M+H]⁺=504.
Step 3: A solution of 3 (1.00 eq) and NaN₃ (2.00 eq) in DMF (3 mL) was stirred at 100° C. for 10 hours. The mixture was diluted with water, then extracted with EtOAc, the organic layer was washed with brine, dried over anhydrous sodium sulfate, filtered and concentrated under reduced pressure. The residue was purified by silica gel chromatography (PE/EA=20/1 to 9/1) to afford 4 as colorless oil. Yield: 60%. LCMS found: [M+H]⁺=375.
Step 4: To a solution of 4 (1.00 eq) in MeOH (3 mL) was added Pd/C (10% purity), the mixture was stirred at 25° C. for 30 min under H2 atmosphere (15 psi). The mixture was filtered through a Celite pad, and the filtrate was concentrated in vacuo to afford 5 as colorless oil. Yield: 72%. LCMS found: [M+H]⁺=349.
Step 4: A solution of 4 (1.00 eq) and HCl (1 mL, 2 N) in THF (3 mL) was stirred at 25° C. for 3 hours. The mixture was concentrated and the residue was purified by reverse phase (C18, 5-40% MeCN in water, 0.1% TFA) to afford A192 as colorless solid. Yield: 68%. LCMS found: [M+H]⁺=309. ¹H NMR (400 MHz, CD₃OD): δ 7.88 (t, J=7.9 Hz, 1H), 7.37 (d, J=7.3 Hz, 1H), 7.06 (d, J=8.4 Hz, 1H), 5.35 (td, J=9.9, 5.3 Hz, 1H), 4.42 (dd, J=10.9, 5.3 Hz, 1H), 4.00 (dd, J=3.4, 1.2 Hz, 1H), 3.88 (dd, J=9.7, 3.4 Hz, 1H), 3.75 (ddd, J=8.0, 3.2, 1.2 Hz, 1H), 3.29-3.25 (m, 2H), 3.18 (dd, J=13.2, 3.2 Hz, 1H).

### Preparation of tert-butyl 4-(4-((2R,3R,4R,5S)-3,4-dihydroxy-5-((6-(trifluoromethyl)pyrazin-2-yl)amino)tetrahydro-2H-pyran-2-yl)-1H-1,2,3-triazol-1-yl)piperidine-1-carboxylate (A193)

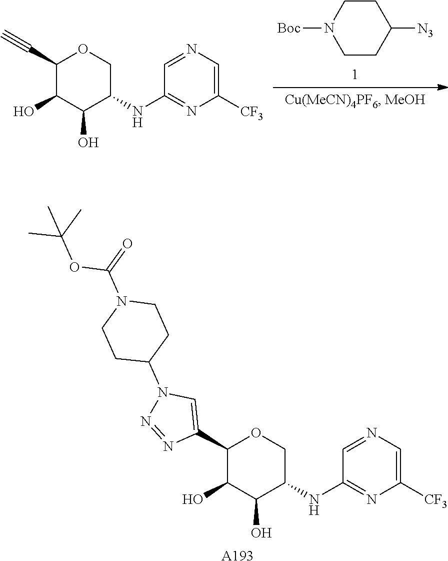

To a solution of (2R,3R,4R,5S)-2-ethynyl-5-((6-(trifluoromethyl)pyrazin-2-yl)amino)tetrahydro-2H-pyran-3,4-diol (1.00 eq) and 1 (2.00 eq) in MeOH (2 mL) was added Cu(MeCN)₄PF₆ (3.00 eq) at 25° C. The mixture was stirred at 25° C. for 3 hours under N₂ atmosphere. LCMS showed the desired mass was detected. The mixture was concentrated under reduced pressure, the residue was diluted with water, then extracted with EtOAc, the organic layer was washed with saturated solution of NaHCO₃ and brine, dried with anhydrous Na₂SO₄, filtered and concentrated under reduced pressure. The crude product was purified by prep-HPLC to give A193 as a white solid.

Yield: 6.3%. LCMS found: [M+H]⁺=530. ¹H NMR (400 MHz, CD₃OD): δ 8.14 (s, 1H), 8.04-7.98 (m, 2H), 4.75-4.69 (m, 2H), 4.45 (td, J=10.6, 5.3 Hz, 1H), 4.25-4.19 (m, 3H), 4.09 (d, J=2.7 Hz, 1H), 3.85 (dd, J=10.6, 3.0 Hz, 1H), 3.27 (t, J=10.9 Hz, 1H), 3.09-2.95 (m, 2H), 2.21-2.14 (m, 2H), 2.03-1.95 (m, 2H), 1.48 (s, 9H). ¹⁹F NMR (377 MHz, CD₃OD) δ −70.32.

### Preparation of 2-(((2R,3R,4R,5S)-3,4-dihydroxy-5-((6-(trifluoromethyl)pyrazin-2-yl)amino)tetrahydro-2H-pyran-2-yl)methoxy)pyrimidine-5-carboxylic acid (A194)

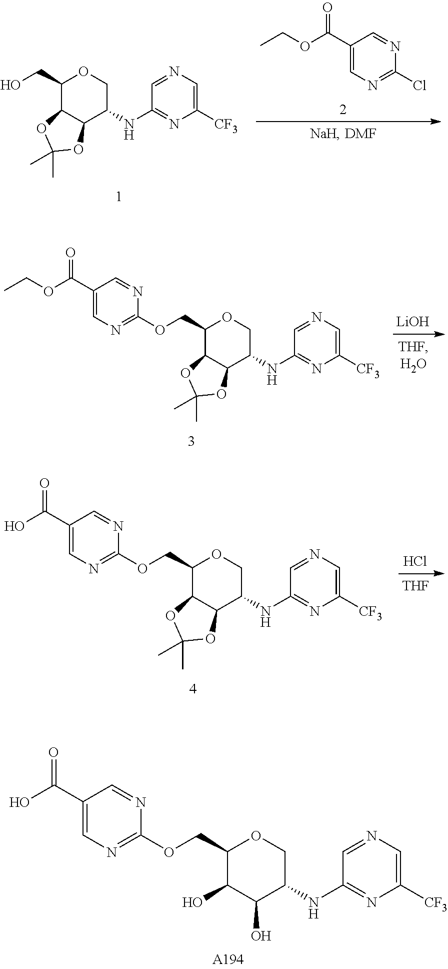

Step 1: To a solution of 1 (1 g, 1.00 eq) in DMF (10 mL) was added NaH (230 mg, 2.00 eq, 60% purity) at 0° C., the mixture was stirred at 0° C. for 0.5 h, then 2 (534 mg, 1.00 eq) was added, the resulting mixture was stirred at 25° C. for 16 h. After completion, the mixture was quenched with saturated solution of NH₄Cl, then extracted with EtOAc, the organic layer was washed with brine, dried over anhydrous sodium sulfate, filtered and concentrated under reduced pressure. The residue was purified by silica gel chromatography (DCM/MeOH=50/1 to 20/1) to give 3 (450 mg) as yellow oil. Yield: 31.5%. LCMS found: [M+H]⁺=500.
Step 2: To a solution of 3 (450 mg, 1.00 eq) in THF (5 mL) and H₂O (1 mL) was added LiOH (108 mg, 5.00 eq), the mixture was stirred at 25° C. for 16 h. LCMS showed the desired mass was observed. The pH of the solution was adjusted to 5 by HCl (2N), then the mixture was concentrated under reduced pressure to afford 4 (crude) as a white solid. LCMS found: [M+H]⁺=472.
Step 3: To a solution of 4 (crude) in THF (5 mL) was added HCl (2 mL, 2N), the mixture was stirred at 25° C. for 2 h. LCMS showed the reaction was completed. The mixture was concentrated under reduced pressure, the residue was purified by reverse phase (5-40% MeCN in water, 0.1% TFA) to give A194 (140 mg, formate) as yellow solid. Yield: 36.0% over two steps. LCMS found: [M+H]⁺=432, ¹H NMR (400 MHz, CD₃OD) δ 9.03 (s, 2H), 8.47 (brs, 2H, formic acid), 8.12 (s, 1H), 8.00 (s, 1H), 4.61 (qd, J=11.2, 6.1 Hz, 2H), 4.38 (td, J=10.7, 5.3 Hz, 1H), 4.15 (dd, J=11.0, 5.1 Hz, 1H), 4.04 (d, J=2.6 Hz, 1H), 3.88 (t, J=6.5 Hz, 1H), 3.71 (dd, J=10.5, 3.1 Hz, 1H), 3.17 (t, J=10.8 Hz, 1H).

### Preparation of tert-butyl 6-(((3S,4R,5R,6R)-4,5-dihydroxy-6-(hydroxymethyl)tetrahydro-2H-pyran-3-yl)amino)-3′,6′-dihydro-[2,4′-bipyridine]-1′(2′H)-carboxylate (A195)

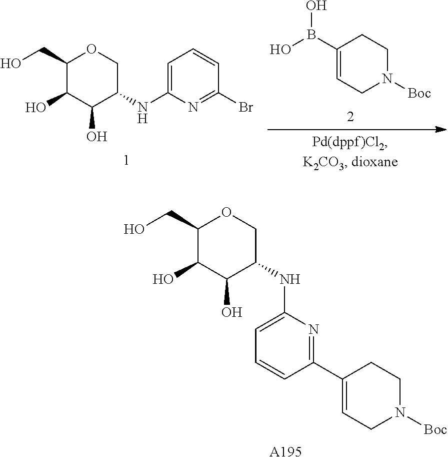 [SMILES: OC[C@H]1OC[C@H](Nc2cccc(Br)n2)[C@@H](O)[C@H]1O ; CC(C)(C)OC(=O)N1CC=C(B(O)O)CC1 ; CC(C)(C)OC(=O)N1CC=C(c2cccc(N[C@H]3CO[C@H](CO)[C@H](O)[C@@H]3O)n2)CC1 ; "A195"]

To a solution of 1 (1.00 eq) and 2 (3.00 eq) in dioxane and water was added Pd(dppf)C12 (0.2 eq) and K₂CO₃ (2.00 eq), the mixture was degassed and purged with nitrogen for 3 times, then stirred at 100° C. for 16 hours under nitrogen atmosphere. LCMS showed the desired mass was observed. The mixture was concentrated under reduced pressure, the residue was purified by reverse phase (5-35% MeCN in water, 0.1% TFA) to give A195 as a yellow solid. Yield: 20%. LCMS found: [M+H]⁺=422.3. ¹H NMR (400 MHz, CD₃OD) δ 7.37 (t, J=8.0 Hz, 1H), 6.68 (d, J=8.0 Hz, 1H) 6.65-6.58 (m, 1H), 6.44 (d, J=8.0 Hz, 1H), 4.32-4.22 (m, 1H), 4.17 (dd, J=10.8, 5.2 Hz, 1H) 4.09-4.04 (m, 2H), 3.90 (d, J=2.8 Hz, 1H), 3.77 (dd, J=11.6, 7.2 Hz, 1H), 3.70 (dd, J=11.6, 5.2 Hz, 1H), 3.64-3.53 (m, 3H), 3.48-3.42 (m, 1H), 3.16 (t, J=10.8 Hz, 1H), 2.64-2.49 (m, 2H), 1.48 (s, 9H).

### Preparation of (2R,3R,4R,5S)-2-(([1,1′-biphenyl]-4-yloxy)methyl)-5-((6-(trifluoromethyl)pyridin-2-yl)amino)tetrahydro-2H-pyran-3,4-diol (A196)

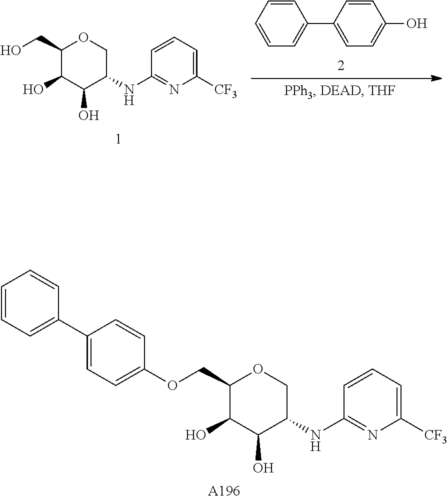 [SMILES: OC[C@H]1OC[C@H](Nc2cccc(C(F)(F)F)n2)[C@@H](O)[C@H]1O ; Oc1ccc(-c2ccccc2)cc1 ; O[C@@H]1[C@H](O)[C@@H](Nc2cccc(C(F)(F)F)n2)CO[C@@H]1COc1ccc(-c2ccccc2)cc1 ; "A196"]

To a solution of 1 (1.00 eq), PPh₃ (3.00 eq) and DEAD (3.00 eq) in THF (5 mL) was added 2 (1.50 eq) in THF (1 mL) dropwise at 25° C. under N₂, the resulting mixture The reaction was stirred at 70° C. for 2 hours under N₂ atmosphere. LCMS showed the desired mass was detected. The mixture was concentrated under reduced pressure, the residue was purified by prep-HPLC (FA condition) to give A196 as a white solid. Yield: 1.85%. LC-MS (ESI) found: 461 [M+H]⁺. ¹H NMR (400 MHz, CD₃OD) δ 7.62-7.47 (m, 5H), 7.39 (t, J=7.2 Hz, 2H), 7.27 (tt, J=7.6, 1.2 Hz, 1H), 7.07-6.98 (m, 2H), 6.89 (d, J=7.2 Hz, 1H), 6.74 (d, J=8.4 Hz, 1H), 4.35 (td, J=10.6, 5.2 Hz, 1H) 4.26-4.17 (m, 3H), 4.04 (d, J=2.8 Hz, 1H), 3.84 (td, J=6.0, 1.2 Hz, 1H), 3.68 (dd, J=10.6, 3.2 Hz, 1H), 3.19 (t, J=10.8 Hz, 1H).

### Preparation of (2R,3R,4R,5S)-5-((9H-purin-2-yl)amino)-2-(hydroxymethyl)tetrahydro-2H-pyran-3,4-diol (A197)

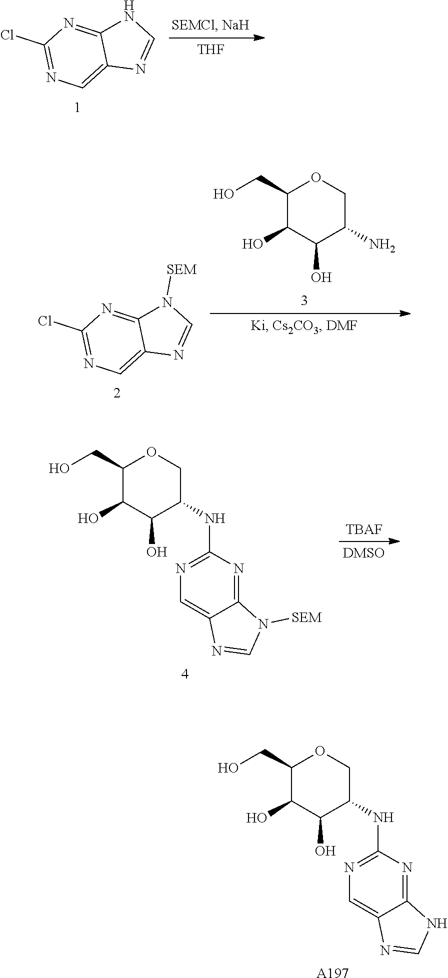

Step 1: To a solution of 1 (1.00 eq) in THF (2 mL) was added NaH (1.50 eq) at 0° C. The mixture was stirred at 0° C. for 30 min, then SEMCl (2.00 eq) was added, the mixture was stirred at 25° C. for 12 hours. LCMS showed the desired mass was detected. The mixture was quenched with saturated solution of NH₄Cl, then extracted with EtOAc, the organic layer was concentrated and the residue was purified by flash chromatography (silica gel, 0˜50% EtOAc in PE) to give 2 as a yellow oil. Yield: 50.5%. LC-MS (ESI) found: 285 [M+H]⁺
Step 2: A solution of 3 (1.00 eq), 2 (1.20 eq), Cs₂CO₃ (2.00 eq) and KI (0.1 eq) in DMF (2 mL) was stirred at 120° C. for 12 hours. The mixture was concentrated in vacuo. The residue was purified by reverse phase (C18, 5-45% MeCN in water, 0.1% TFA) to afford 4 as a brown oil. Yield: 40.12%. LC-MS (ESI) found: 412 [M+H]⁺
Step 3: To a solution of 4 (1.00 eq) in DMSO (2 mL) was added TBAF (1.00 eq), the mixture was stirred at 120° C. for 12 hours. The solution was purified by reverse phase (C18, 0-20% MeCN in water, 0.1% TFA) directly without any work up to afford A197 as a white solid. Yield: 16.26%. LC-MS (ESI) found: 282 [M+H]⁺. ¹H NMR (400 MHz, CD₃OD): δ 8.67 (s, 1H), 8.29 (s, 1H), 4.43-4.33 (m, 1H), 4.20 (dd, J=10.9, 5.1 Hz, 1H), 3.94 (d, J=2.9 Hz, 1H), 3.77 (dd, J=11.6, 7.2 Hz, 1H), 3.74-3.67 (m, 2H), 3.51-3.46 (m, 1H), 3.21 (d, J=10.8 Hz, 1H).

### Preparation of (2R,3R,4S,5S)-2-((4-bromo-3-fluorophenoxy)methyl)-5-((6-(trifluoromethyl)pyridin-2-yl)oxy)tetrahydro-2H-pyran-3,4-diol (A198)

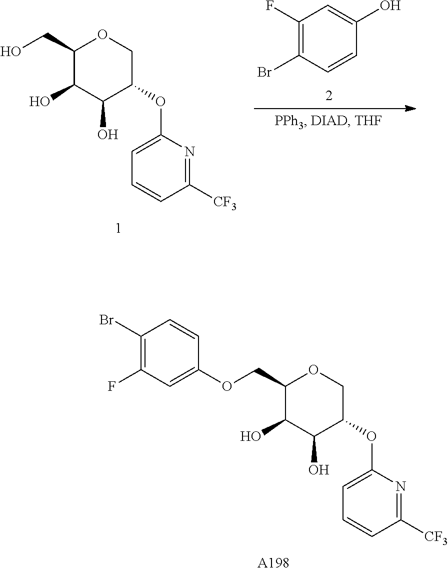 [SMILES: OC[C@H]1OC[C@H](Oc2cccc(C(F)(F)F)n2)[C@@H](O)[C@H]1O ; Oc1ccc(Br)c(F)c1 ; O[C@@H]1[C@H](O)[C@@H](Oc2cccc(C(F)(F)F)n2)CO[C@@H]1COc1ccc(Br)c(F)c1 ; "A198"]

To a solution of 1 (1.00 eq), 2 (1.20 eq) and PPh₃ (4.00 eq) in THF (3 mL) was added DIAD (4.00 eq) dropwise at 0° C., the resulting mixture was stirred at 60° C. for 3 hours under nitrogen atmosphere. The mixture was concentrated in vacuo. The residue was purified by reverse phase (C18, 5-70% MeCN in water, 0.1% TFA) to afford A198 as white solid. Yield: 22%. LCMS found: [M+H]⁺=482, 484. ¹H NMR (400 MHz, CD₃OD): δ 7.87 (t, J=8.0 Hz, 1H), 7.47 (t, J=8.4 Hz, 1H), 7.36 (d, J=7.1 Hz, 1H), 7.05 (d, J=8.4 Hz, 1H), 6.87 (dd, J=10.4, 2.8 Hz, 1H), 6.75 (ddd, J=9.2, 2.8, 1.2 Hz, 1H), 5.38 (td, J=10.0, 5.6 Hz, 1H), 4.37 (dd, J=10.8, 5.6 Hz, 1H), 4.18 (d, J=6.4 Hz, 2H), 4.07 (dd, J=3.2, 1.2 Hz, 1H), 3.95-3.81 (m, 2H), 3.27 (d, J=10.8 Hz, 1H).

### Preparation of 4-((4-(((2R,3R,4R,5S)-3,4-dihydroxy-5-((6-(trifluoromethyl)pyrazin-2-yl)amino)tetrahydro-2H-pyran-2-yl)methyl)piperazin-1-yl)methyl)benzoic acid (A199)

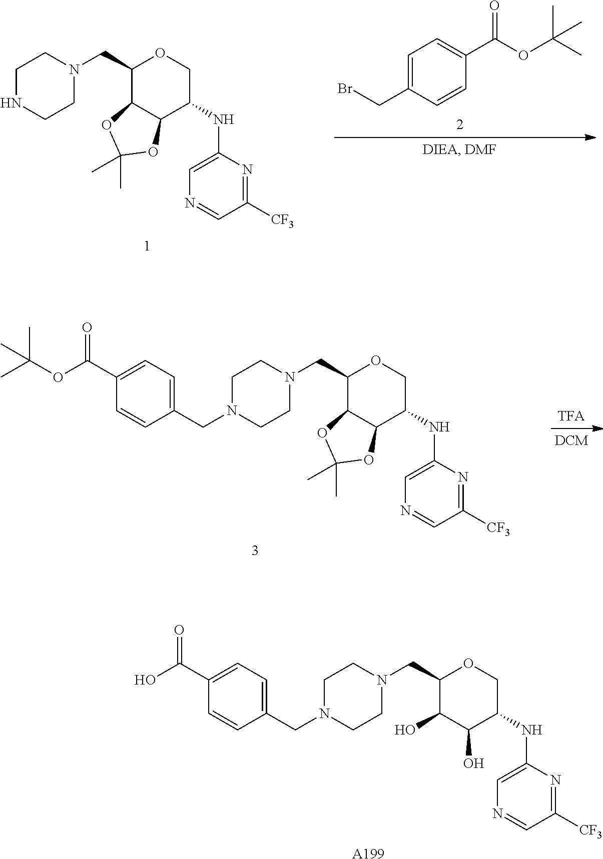 [SMILES: CC1(C)O[C@@H]2[C@H](O1)[C@@H](Nc1cncc(C(F)(F)F)n1)CO[C@@H]2CN1CCNCC1 ; CC(C)(C)OC(=O)c1ccc(CBr)cc1 ; CC(C)(C)OC(=O)c1ccc(CN2CCN(C[C@H]3OC[C@H](Nc4cncc(C(F)(F)F)n4)[C@H]4OC(C)(C)O[C@H]43)CC2)cc1 ; "3" ; O=C(O)c1ccc(CN2CCN(C[C@H]3OC[C@H](Nc4cncc(C(F)(F)F)n4)[C@@H](O)[C@H]3O)CC2)cc1 ; "A199"]

Step 1: To a solution of 1 (1.00 eq) in DMF (3 mL) was added DIEA (3.00 eq) and 2 (1.50 eq). The mixture was stirred at 80° C. for 3 h. After completion, the mixture was diluted with brine and extracted with EtOAc, the organic layer was dried over anhydrous sodium sulfate, filtered and concentrated under reduced pressure. The residue was purified by flash (DCM/MeOH=50/1 to 10/1) to afford 3 as brown oil. LCMS found: [M+H]⁺=608.
Step 2: To a solution of 3 (1.00 eq) in DCM (2 mL) was added TFA (2 mL), the mixture was stirred at 25° C. for 12 hours. LCMS showed the starting material was consumed completely, one major peak with desired mass was detected. The mixture was concentrated in vacuo. The residue was purified by reverse flash (5-27% MeCN in water, 0.1% TFA) to afford A199 (TFA salt) as a light yellow solid. Yield: 40.8%. LCMS found: [M+H]⁺=512. ¹H NMR (400 MHz, CD₃OD): δ 8.13 (s, 1H), 8.03-8.00 (m, 3H), 7.49 (d, J=8.0 Hz, 2H), 4.34 (td, J=10.8, 5.2 Hz, 1H), 4.20 (dd, J=12.0, 5.2 Hz, 1H), 3.92-3.85 (m, 2H), 3.79 (s, 2H), 3.71 (dd, J=10.8, 3.2 Hz, 1H), 3.52 (dd, J=13.6, 9.6 Hz, 1H), 3.42-3.33 (m, 3H), 3.25 (dd, J=13.6, 2.8 Hz, 2H), 3.18 (t, J=10.8 Hz, 1H) 2.95-2.68 (m, 4H). ¹⁹F NMR (377 MHz, CD₃OD) δ −70.36, -77.41.

### Preparation of (2R,3R,4R,5S)-2-(1-(piperidin-4-yl)-1H-1,2,3-triazol-4-yl)-5-((6-(trifluoromethyl)pyrazin-2-yl)amino)tetrahydro-2H-pyran-3,4-diol (A200)

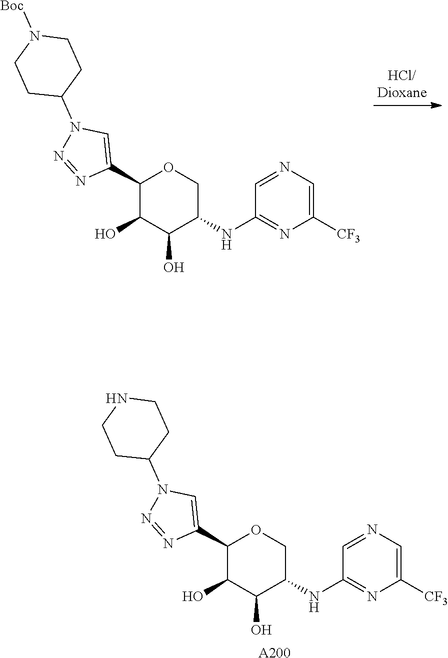

The solution of tert-butyl 4-(4-((2R,3R,4R,5S)-3,4-dihydroxy-5-((6-(trifluoromethyl)pyrazin-2-yl)amino)tetrahydro-2H-pyran-2-yl)-1H-1,2,3-triazol-1-yl)piperidine-1-carboxylate (1.00 eq) in HCl/1,4-dioxane (2 mL) was stirred at 25° C. for 1 hour. LCMS showed the desired mass was detected. The mixture was concentrated in vacuo. The crude product was purified by prep-HPLC to give A200 as a yellow solid. Yield: 90%. LCMS found: [M+H]⁺=430. ¹H NMR (400 MHz, CD₃OD): δ 8.47 (s, 1H), 8.39-8.13 (m, 2H), 5.08 (t, J=6.4 Hz, 1H), 4.95 (s, 1H), 4.52-4.45 (m, 1H), 4.26 (dd, J=10.9, 4.8 Hz, 1H), 4.15 (s, 1H), 3.93 (dd, J=10.0, 1.6 Hz, 1H), 3.65-3.57 (m, 2H), 3.40-3.32 (m, 2H), 3.30-3.25 (m, 1H), 2.57-2.49 (m, 2H), 2.47-2.36 (m, 2H). ¹⁹F NMR (377 MHz, CD₃OD) δ −70.40.

### Preparation of (2R,3R,4R,5S)-2-(1-phenyl-1H-1,2,3-triazol-4-yl)-5-((6-(trifluoromethyl)pyrazin-2-yl)amino)tetrahydro-2H-pyran-3,4-diol (A201)

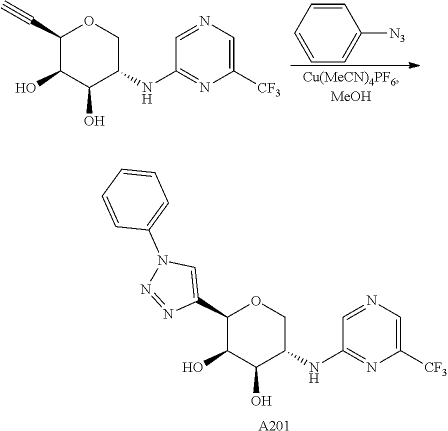

To a solution of (2R,3R,4R,5S)-2-ethynyl-5-((6-(trifluoromethyl)pyrazin-2-yl)amino)tetrahydro-2H-pyran-3,4-diol (1.00 eq) and azidobenzene (2.00 eq) in MeOH (2 mL) was added Cu(MeCN)₄PF₆ (3.00 eq), the reaction was stirred at 25° C. for 3 hours under N₂ atmosphere. LCMS showed the desired mass was detected. The reaction was concentrated under reduced pressure, the residue was purified by prep-HPLC to give A201 as a white solid. Yield: 35.9%. LCMS found: [M+H]⁺=423. ¹H NMR (400 MHz, CD₃OD): δ 8.47 (s, 1H), 8.15 (s, 1H), 8.03 (s, 1H), 7.88-7.83 (m, 2H), 7.61-7.57 (m, 2H), 7.50 (tt, J=7.6, 2.0 Hz, 1H), 4.85-4.84 (m, 1H), 4.48 (td, J=10.6, 5.2 Hz, 1H), 4.27 (dd, J=11.2, 5.2 Hz, 1H), 4.18 (d, J=2.4 Hz, 1H), 3.89 (dd, J=10.8, 3.2 Hz, 1H), 3.35 (t, J=10.8 Hz, 1H). ¹⁹F NMR (377 MHz, CD₃OD) δ −70.32.

### Preparation of (2R,3R,4R,5S)-2-(([1,1′-biphenyl]-4-yloxy)methyl)-5-((6-(trifluoromethyl) pyrazin-2-yl)amino)tetrahydro-2H-pyran-3,4-diol (A202)

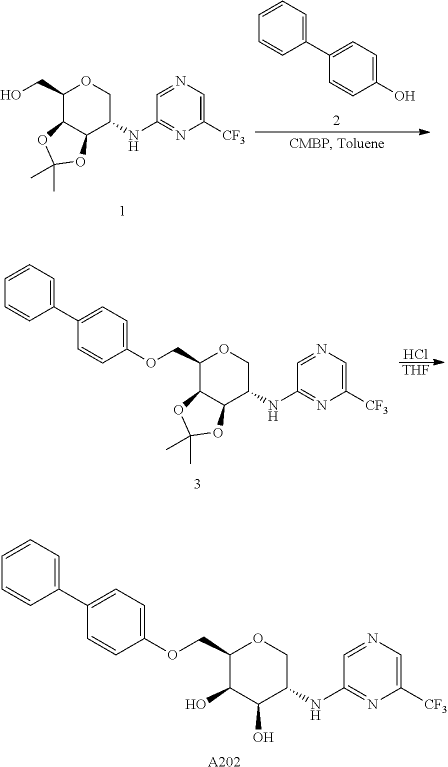

Step 1: To a solution of 1 (1.00 eq) and 2 (1.20 eq) in toluene (10 mL) was added CMBP (2.00 eq) at 25° C. The reaction was stirred at 100° C. for 12 hours. LCMS showed the desired mass was detected. The resulting mixture was filtered and the filtrate was concentrated in vacuo to give crude 3 as a colorless oil. LC-MS (ESI) found: 502 [M+H]⁺.
Step 2: The solution of 3 (1.00 eq) and HCl (2.00 eq, 2N) in THF (10 mL) was stirred at 25° C. for 12 hours. LCMS showed the desired mass was detected. The mixture was concentrated under reduced pressure, the crude product was purified by prep-HPLC to give A202 as a white solid. Yield: 47.8% over two steps. LCMS found: [M+H]⁺=462. ¹H NMR (400 MHz, CD₃OD) δ 8.12 (s, 1H), 8.01 (s, 1H), 7.57-7.53 (m, 4H), 7.39 (t, J=7.7 Hz, 2H), 7.27 (tt, J=7.6, 1.2 Hz, 1H) 7.03 (d, J=8.8 Hz, 2H), 4.40 (td, J=10.8, 5.2 Hz, 1H), 4.23-4.15 (m, 3H), 4.06 (d, J=2.8 Hz, 1H), 3.85 (t, J=6.4 Hz, 1H), 3.72 (dd, J=10.8, 3.2 Hz, 1H), 3.20 (t, J=10.8 Hz, 1H).

### Preparation of 5-(((2R,3R,4R,5S)-3,4-dihydroxy-5-((6-(trifluoromethyl)pyrazin-2-yl)amino)tetrahydro-2H-pyran-2-yl)methoxy)pyrimidine-2-carbonitrile (A203)

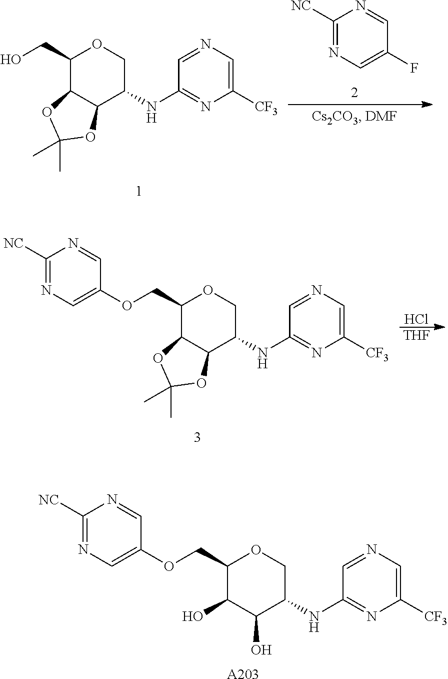

Step 1: To a solution of 1 (1.00 eq) in DMF (10 mL) was added Cs₂CO₃ (1.50 eq) and 2 (1.50 eq), the mixture was stirred at 50° C. for 16 hrs. LCMS showed the major peak with desired mass was observed. The mixture was diluted with water, then extracted with EtOAc, the organic layer was washed with brine, dried over anhydrous sodium sulfate, filtered and concentrate under reduced pressure. The residue was purified by silica gel chromatography (PE/EA=5/1 to 0/1) to give 3 as yellow oil. Yield: 65.2%. LC-MS (ESI) found: 453 [M+H]⁺
Step 2: To a solution of 3 (1.00 eq) in THF (5 mL) was added HCl (2 mL, 3N), the mixture was stirred at 25° C. for 2 hours. LCMS showed the desired mass was detected. The mixture was concentrated in vacuo. The residue was purified by reverse phase (5-33% MeCN in water, 0.1% TFA) to give A203 as a white solid. Yield: 36.57%. LC-MS (ESI) found: 413 [M+H]. ¹H NMR (400 MHz, CD₃OD) δ 8.64 (s, 2H), 8.12 (s, 1H), 8.01 (s, 1H), 4.47 (dd, J=10.4, 7.2 Hz, 1H), 4.42 (dd, J=10.8, 4.8 Hz, 1H), 4.36 (dd, J=10.8, 5.2 Hz, 1H), 4.15 (dd, J=10.8, 5.2 Hz, 1H), 4.02 (dd, J=3.2, 0.8 Hz, 1H), 3.90 (ddd, J=7.2, 4.4, 1.2 Hz, 1H), 3.72 (dd, J=10.4, 3.2 Hz, 1H), 3.17 (t, J=10.8 Hz, 1H).

### Preparation of 4-((4-(((2R,3R,4R,5S)-3,4-dihydroxy-5-((6-(trifluoromethyl)pyrazin-2-yl)amino)tetrahydro-2H-pyran-2-yl)methyl)piperazin-1-yl)methyl)-2-fluorobenzoic acid (A204)

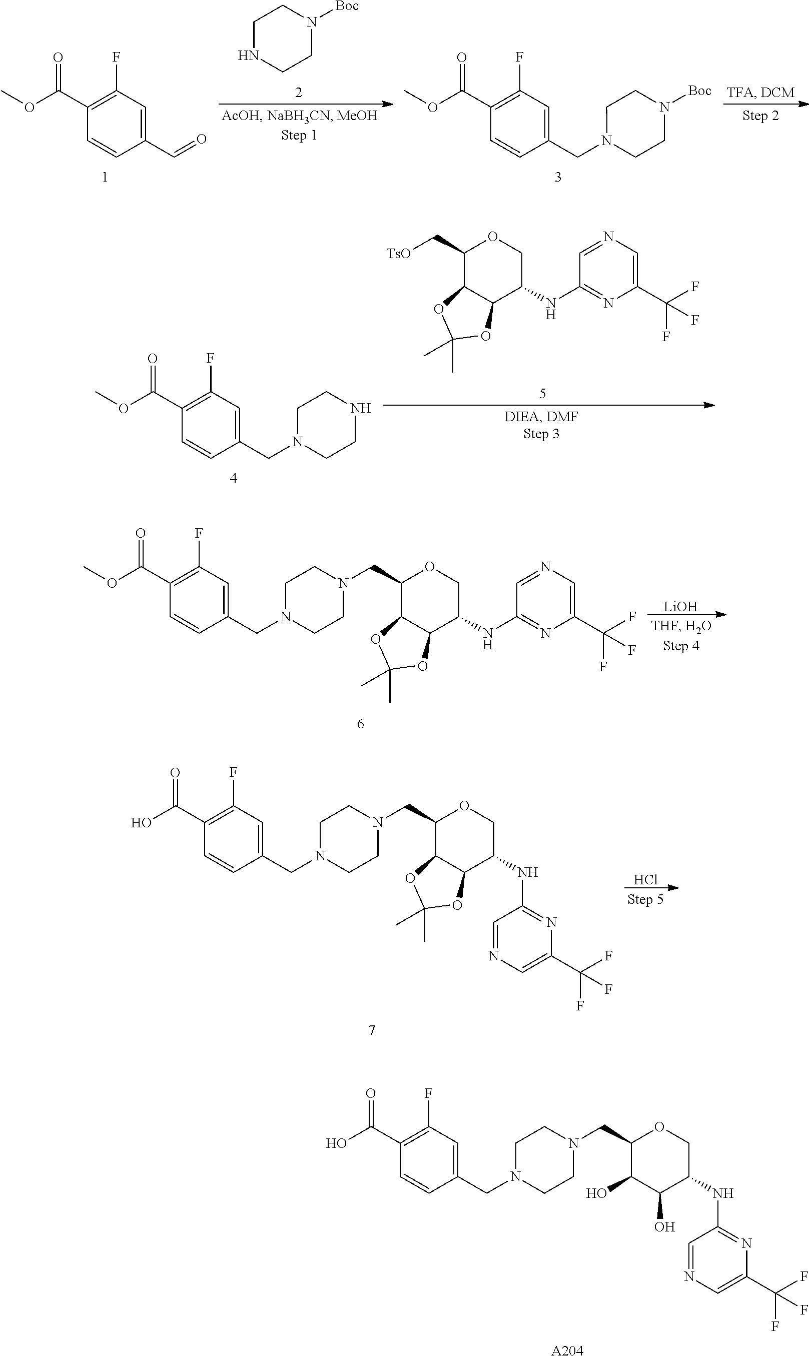

Step 1: The mixture of 1 (1.00 eq), 2 (1.00 eq) and AcOH (0.2 eq) in MeOH was stirred at 20° C. for 1 hour, then NaBH₃CN (1.50 eq) was added, the resulting mixture was stirred at 20° C. for 12 hours. The mixture was quenched with water and extracted with EA, the organic phase was dried over anhydrous sodium sulfate, filtered and concentrated under reduced pressure. The residue was purified by silica gel chromatography (PE/EA=10/1 to 1/1) to afford 3 as a yellow oil. Yield: 79.4%. LCMS found: [M+H]⁺=353.
Step 2: The mixture of 3 (1.00 eq) and TFA (10.00 eq) in DCM was stirred at 20° C. for 4 hours. The mixture was concentrated and purified by reverse phase (5-20% ACN in Water with 0.1% TFA) to afford 4 as a white solid. Yield: 83.8%. LCMS found: [M+H]⁺=253.
Step 3: The mixture of 4 (1.00 eq), 5 (1.00 eq) and DIEA (3.00 eq) in DMF was stirred at 100° C. for 12 hours. The mixture was diluted with EA and washed with water and brine. The organic phase was concentrated and purified by Flash (DCM:MeOH=15:1) to afford 6 as a yellow solid. Yield: 41.7%. LCMS found: [M+H]⁺=584.
Step 4: The mixture of 6 (1.00 eq) and LiOH (3.00 eq) in THE and H₂O was stirred at 20° C. for 12 hours. The pH of the mixture was adjusted to 6 by HCl (1N), the aqueous was extracted with EA, the organic layer was concentrated to give 7 as a yellow solid. Yield: 88.3%. LCMS found: [M+H]⁺=570.
Step 5: The mixture of 7 (1.00 eq) and HCl (1.2 M, 10.00 eq) in THE was stirred at 20° C. for 12 hours. The mixture was concentrated and the residue was purified by reverse phase (5-35% ACN in Water with 0.1% TFA) to afford A204 as a yellow solid. Yield: 86.0%. LCMS found: [M+H]⁺=530. ¹H NMR (400 MHz, CD₃OD) δ 8.12 (s, 1H), 8.02 (s, 1H), 7.92 (t, J=8.0 Hz, 1H), 7.27 (s, 1H), 7.25 (d, J=4.8 Hz, 1H), 4.34 (td, J=10.6, 4.8 Hz, 1H), 4.20 (dd, J=11.2, 5.2 Hz, 1H), 3.92 (d, J=8.8 Hz, 1H), 3.87 (d, J=2.8 Hz, 1H), 3.74 (s, 2H), 3.71 (dd, J=10.4, 3.6 Hz, 1H), 3.57 (dd, J=13.6, 10.0 Hz, 1H), 3.50-3.32 (m, 4H), 3.29-3.26 (m, 1H), 3.19 (t, J=10.8 Hz, 1H) 2.94-2.67 (m, 4H).

### Preparation of 6-((4-(((2R,3R,4R,5S)-3,4-dihydroxy-5-((6-(trifluoromethyl)pyrazin-2-yl)amino)tetrahydro-2H-pyran-2-yl)methyl)piperazin-1-yl)methyl)pyridazine-3-carboxylic acid (A205)

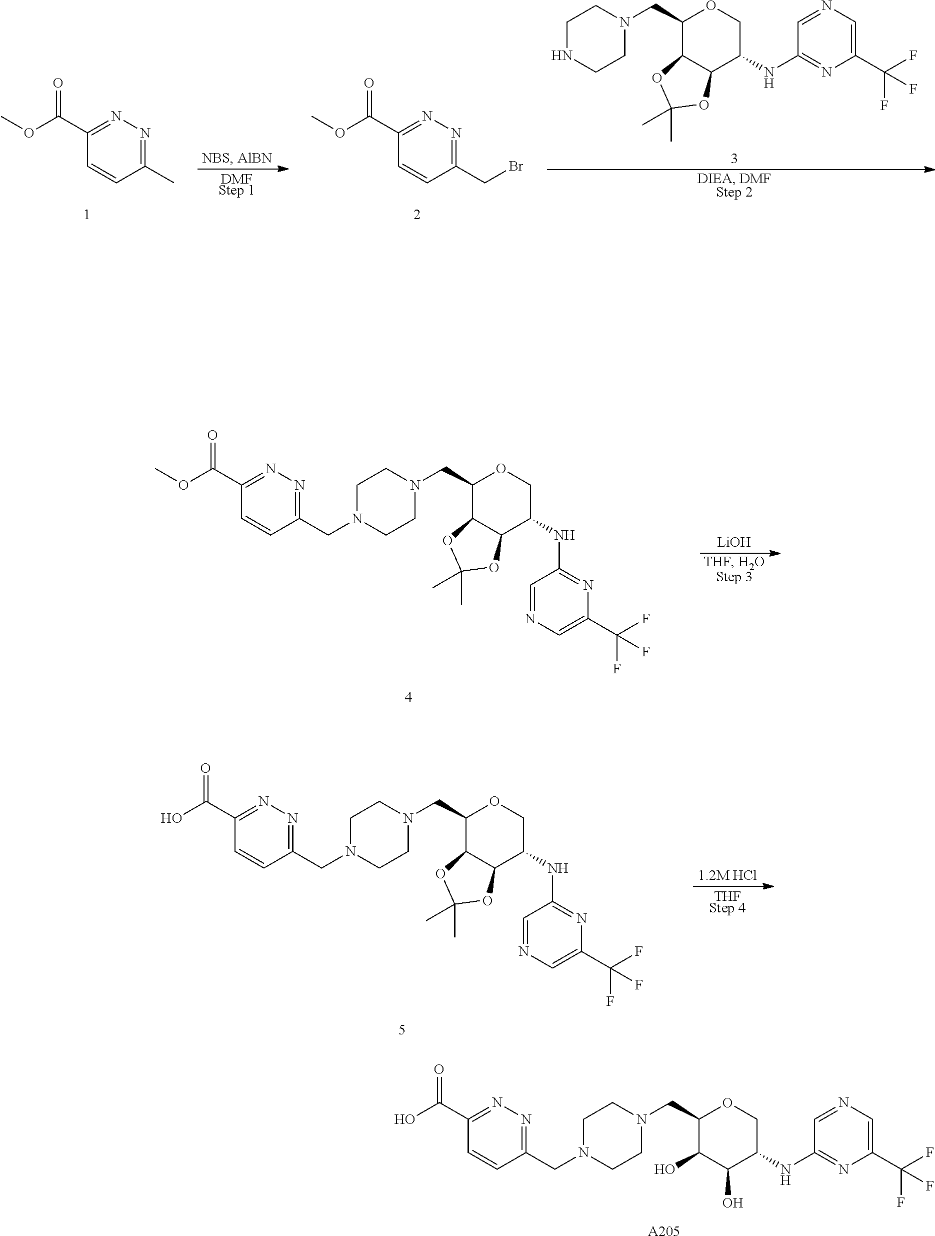

Step 1: To a mixture of 1 (1.00 eq) in DMF was added NBS (1.20 eq) and AIBN (0.1 eq), the mixture was stirred at 25° C. for 12 hours. The mixture was concentrated and the residue was purified by flash (PE/EA=20/1 to 3/1) to give 2 as a yellow solid. Yield: 61.98%. LCMS found: [M+H]⁺=231.
Step 2: The mixture of 2 (1.50 eq), 3 (0.83 eq) and DIEA (3.00 eq) in DMF was stirred at 100° C. for 12 hours. The mixture was diluted with water, extracted with ethyl acetate, the organic layer was washed with brine, dried over anhydrous sodium, filtered and concentrated under reduced pressure, the residue was purified by Flash (DCM:MeOH=20:1) to give 4 as a yellow solid. Yield: 58.01%. LCMS found: [M+H]⁺=568.
Step 3: The mixture of 4 (1.00 eq) and LiOH (3.00 eq) in THE and H₂O was stirred at 20° C. for 12 hours. The pH of the mixture was adjusted to 6 with HCl (1N), the mixture was concentrated in vacuo to give 5 (crude) as a yellow solid. Yield: 97.07%. LCMS found: [M+H]⁺=554.
Step 4: The mixture of 5 (1.00 eq) and 1.2 M HCl (10.00 eq) in THE was stirred at 25° C. for 12 hours. The mixture was concentrated under reduced pressure, the residue was purified by reversed phase (5-25% ACN in Water with 0.1% TFA) to afford A205 as a brown solid. Yield: 92.11%. LCMS found: [M+H]⁺=514. ¹H NMR (400 MHz, CD₃OD) δ 8.32 (d, J=8.4 Hz, 1H), 8.13 (s, 1H), 8.02 (s, 1H), 8.00 (d, J=8.4 Hz, 1H), 4.34 (td, J=10.6, 5.1 Hz, 1H), 4.21 (dd, J=11.0, 5.1 Hz, 1H), 4.10 (s, 2H), 3.94 (d, J=8.5 Hz, 1H), 3.87 (d, J=2.8 Hz, 1H), 3.72 (dd, J=10.5, 3.0 Hz, 1H), 3.60 (dd, J=13.5, 9.9 Hz, 1H), 3.53-3.32 (m, 5H), 3.19 (t, J=10.8 Hz, 1H), 3.00-2.77 (m, 4H).

### Preparation of 2-(4-(4-(((2R,3R,4R,5S)-3,4-dihydroxy-5-((6-(trifluoromethyl)pyrazin-2-yl)amino)tetrahydro-2H-pyran-2-yl)methyl)piperazin-1-yl)piperidin-1-yl)acetic acid (A206)

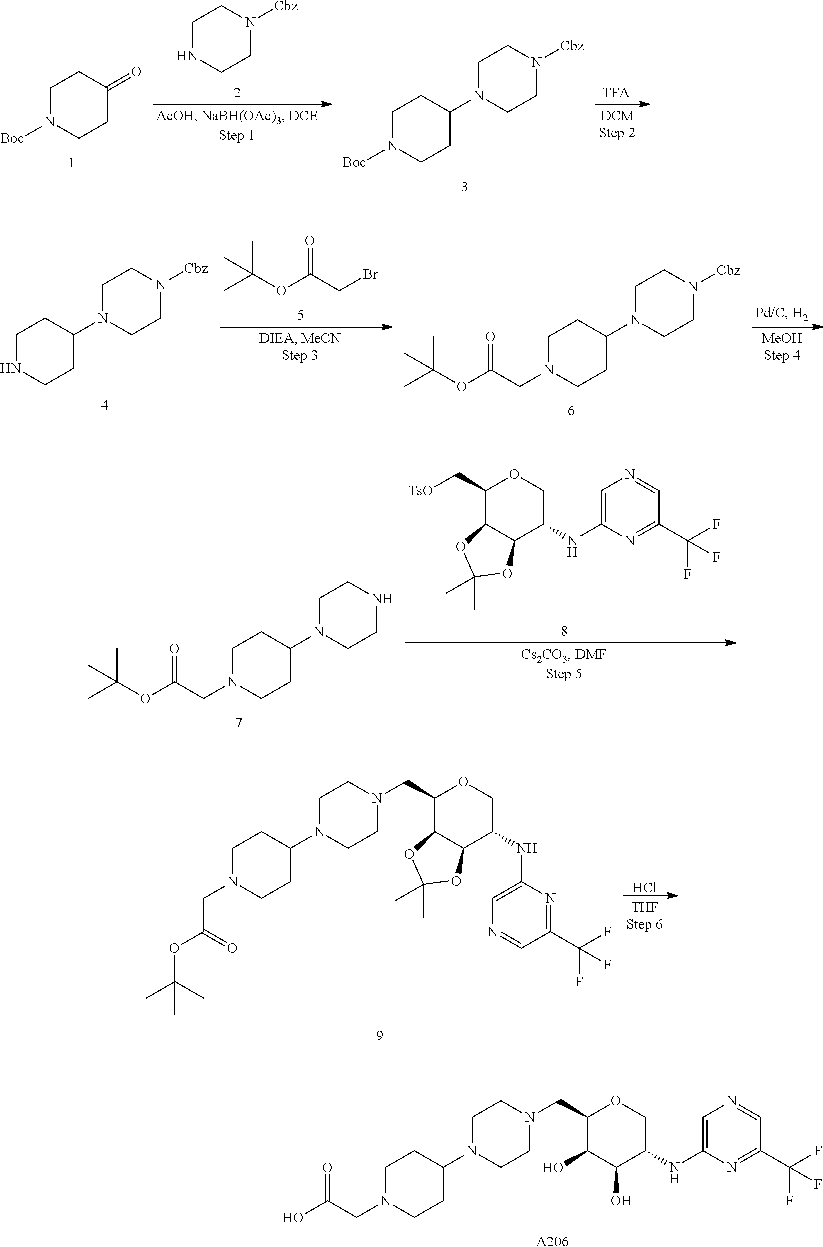

Step 1: To a solution of 1 (1.20 eq) and 2 (1.00 eq) in DCE (10 mL) was added AcOH (0.20 eq) at 25° C. The mixture was stirred at 25° C. for 1 hour, then NaBH(OAc)₃ (3.00 eq) was added. The reaction was stirred at 25° C. for 3 hours. The mixture was quenched with saturated solution of NH₄Cl, extracted with EA, the organic layer was separated and washed with H₂O and brine, dried over Na₂SO₄, filtered and concentrated in vacuo. The crude product was purified by flash chromatography (silica gel, 0˜5% MeOH in DCM) to give 3 as a colorless oil. Yield: 79.0%. LC-MS (ESI) found: 404 [M+H]⁺.
Step 2: To a solution of 3 (1.00 eq) in DCM (10 mL) was added TFA (10.00 eq) at 0° C. The reaction was stirred at 25° C. for 1.5 hour. LCMS showed the desired mass was detected. The mixture was concentrated in vacuo to give 4 as a yellow oil. Yield: 61.3%. LC-MS (ESI) found: 304 [M+H]⁺.
Step 3: To a solution of 4 (1.00 eq) and 5 (1.00 eq) in CH₃CN (10 mL) was added DIEA (5.00 eq). The reaction was stirred at 60° C. for 2 hours. LCMS showed the desired mass was detected. The mixture was concentrated in vacuo. The crude product was purified by flash chromatography (silica gel, 0˜5% MeOH in DCM) to give 6 as a colorless oil. Yield: 72.8%. LC-MS (ESI) found: 418 [M+H]⁺.
Step 4: To a solution of 6 (1.00 eq)) in MeOH (10 mL) was added Pd/C (10% purity) at 25° C. The reaction was stirred at 25° C. for 2 hours under H2 (15 Psi) atmosphere. LCMS showed the desired mass was detected. The resulting mixture was filtered and the filtrate was concentrated in vacuo to give 7 as a colorless oil. Yield: 68.2%. LC-MS (ESI) found: 284 [M+H]⁺.
Step 5: To a solution of 7 (1.00 eq) and 8 (1.00 eq) in DMF (7 mL) was added Cs₂CO₃ (3.00 eq) at 25° C. The reaction was stirred at 100° C. for 18 hours. LCMS showed the desired mass was detected. The mixture was diluted with water, then extracted with EA, the organic layer was washed with brine, dried over Na₂SO₄, filtered and concentrated in vacuo. The crude product was purified by flash chromatography (silica gel, 0˜35% MeOH in DCM) to give 9 as a yellow oil. Yield: 35.7%. LC-MS (ESI) found: 615 [M+H]⁺.
Step 6: To a solution of 9 (1.00 eq) in THF (4 mL) was added 2N HCl (4 mL). The reaction was stirred at 40° C. for 4 hours. LCMS showed the desired mass was detected. The mixture was concentrated in vacuo. The crude product was purified by prep-HPLC to give A206 as a yellow solid. Yield: 71.3%. LCMS found: [M+H]⁺=519. ¹H NMR (400 MHz, CD₃OD): δ 8.14 (s, 1H), 8.03 (s, 1H), 4.35 (td, J=10.6, 5.1 Hz, 1H), 4.21 (dd, J=11.1, 5.1 Hz, 1H), 4.07 (s, 2H), 4.01-3.93 (m, 1H), 3.89 (d, J=2.4 Hz, 1H), 3.75-3.64 (m, 3H), 3.57 (dd, J=13.6, 9.8 Hz, 1H), 3.50-3.33 (m, 5H), 3.24-3.12 (m, 3H), 3.05-2.81 (m, 4H), 2.76-2.69 (m, 1H), 2.19-2.08 (m, 2H), 1.99-1.83 (m, 2H).

### Preparation of 6-(4-(((2R,3R,4R,5S)-3,4-dihydroxy-5-((6-(trifluoromethyl)pyrazin-2-yl)amino)tetrahydro-2H-pyran-2-yl)methyl)piperazin-1-yl)pyridazine-3-carboxylic acid (A207)

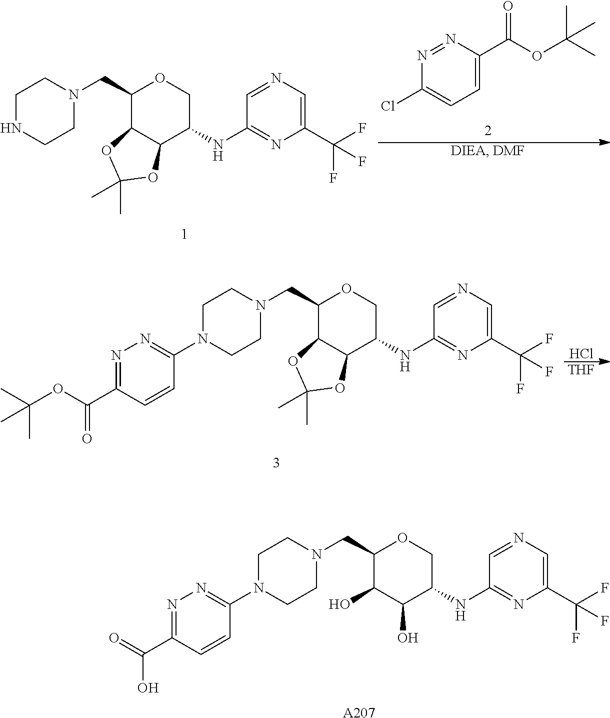

Step 1: To a solution of 1 (1.00 eq) and 2 (1.00 eq) in DMF was added DIEA (3.00 eq), the mixture was stirred at 80° C. for 16 hours. LCMS showed the reaction was completed. The mixture was diluted with water, extracted with ethyl acetate, the organic layer was washed with water and brine, then dried over anhydrous sodium sulfate, filtered and concentrated under reduced pressure to give 3 as yellow oil. Yield: 28.03%. LC-MS (ESI) found: [M+H]⁺=596.
Step 2: To a solution of 3 (1.00 eq) in THE was added HCl (1 mL, 2 N) at 25° C. The reaction mixture was stirred at 25° C. for 2 hours. LCMS showed the reaction was completed. The mixture was concentrated under reduced pressure and the residue was purified by prep-HPLC (TFA condition) to afford A207 as pink solid. Yield: 23.30%. LC-MS (ESI) found: [M+H]⁺=500. ¹H NMR (400 MHz, CD₃OD) δ 8.12 (s, 1H), 8.03-7.96 (m, 2H), 7.34 (d, J=9.4 Hz, 1H), 4.39-4.29 (m, 1H), 4.19-4.11 (m, 1H), 4.06-3.94 (m, 4H), 3.91-3.83 (m, 2H), 3.69 (d, J=10.6 Hz, 1H), 3.37-3.33 (m, 1H), 3.28-3.21 (m, 4H), 3.18-3.10 (m, 2H).

### Preparation of (2R,3R,4S,5R,6S)-2-(hydroxymethyl)-6-phenyl-5-((6-(trifluoromethyl)pyridin-2-yl)oxy)tetrahydro-2H-pyran-3,4-diol (A208)

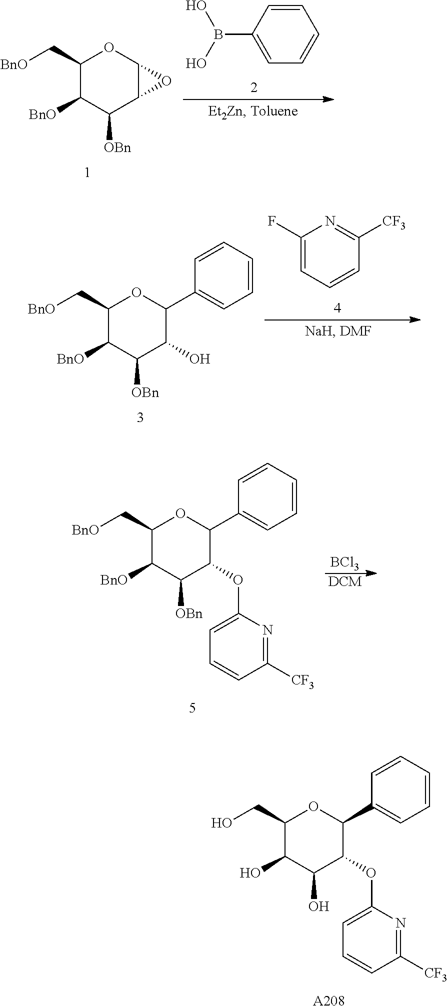

Step 1: To a solution of 2 (1.10 eq) in Toluene (8 mL) was added Et₂Zn (3.30 eq) at 25° C. The mixture was stirred at 60° C. for 1 hour. The mixture was cooled to 25° C. and 1 (1.00 eq) was added. The reaction was stirred at 60° C. for 2 hours. The reaction was quenched with Ammonium chloride solution. The reaction was diluted with EA (100 mL), washed with H₂O (30 mL*2) and brine (30 mL). The organic layer was separated, dried over Na₂SO₄ and concentrated in vacuo. The crude product was purified by flash chromatography (silica gel, 0˜30% EA in PE) to give 3 as a colorless oil. Yield: 27.1%. ¹H NMR (400 MHz, CDCl₃): δ 7.39-7.24 (m, 20H), 5.04 (s, 1H), 4.82 (d, J=12.0 Hz, 1H), 4.63 (q, J=11.6 Hz, 3H), 4.56 (s, 2H), 4.45 (ddd, J=8.7, 5.9, 2.8 Hz, 1H), 4.20 (dd, J=11.5, 8.8 Hz, 1H), 4.13 (dd, J=5.9, 2.2 Hz, 1H), 3.98 (s, 2H), 3.83 (dd, J=11.5, 2.9 Hz, 1H), 1.64 (d, J=3.3 Hz, 1H).
Step 2: To a solution of 3 (1.00 eq) and 4 (1.10 eq) in DMF was added NaH (3.00 eq, 60% purity) at room temperature. The reaction mixture was stirred at 25° C. for 16 hours. LCMS showed the reaction was completed. The mixture was quenched with saturated solution of NH₄Cl, extracted with ethyl acetate, the organic layer was dried over anhydrous sodium sulfate, filtered and concentrated under reduced pressure, the residue was purified by silica gel chromatography (PE:EA=5:1 to 1:1) to give 5 as a yellow oil. Yield: 30.03%. LC-MS (ESI) found: [M+H]⁺=656.
Step 3: To a solution of 5 (1.00 eq) in DCM was added BCl₃ (3.00 eq) at 0° C. The mixture was stirred at 25° C. for 0.5 hour. LCMS showed the reaction was completed. The mixture was quenched with Et₃N, then concentrated under reduced pressure. The residue was purified by prep-HPLC to afford A208 as yellow oil. Yield: 25.61%. LC-MS (ESI) found: [M+H]⁺=386. ¹H NMR (400 MHz, CD₃OD) δ 7.60 (t, J=8.0 Hz, 1H), 7.34-7.29 (m, 2H), 7.16 (d, J=7.6 Hz, 1H), 7.11-7.00 (m, 2H), 6.62 (d, J=8.4 Hz, 1H), 5.73 (dd, J=5.2, 3.2 Hz, 1H), 5.28 (d, J=5.2 Hz, 1H), 4.50 (dd, J=6.4, 3.2 Hz, 1H), 3.93-3.86 (m, 2H), 3.78 (dd, J=11.2, 5.2 Hz, 1H), 3.69 (dd, J=11.2, 6.4 Hz, 1H).

### Preparation of (2R,3R,4R,5S)-2-((5-phenyl-1,3,4-oxadiazol-2-yl)methyl)-5-((6-(trifluoromethyl)pyrazin-2-yl)amino)tetrahydro-2H-pyran-3,4-diol (A209)

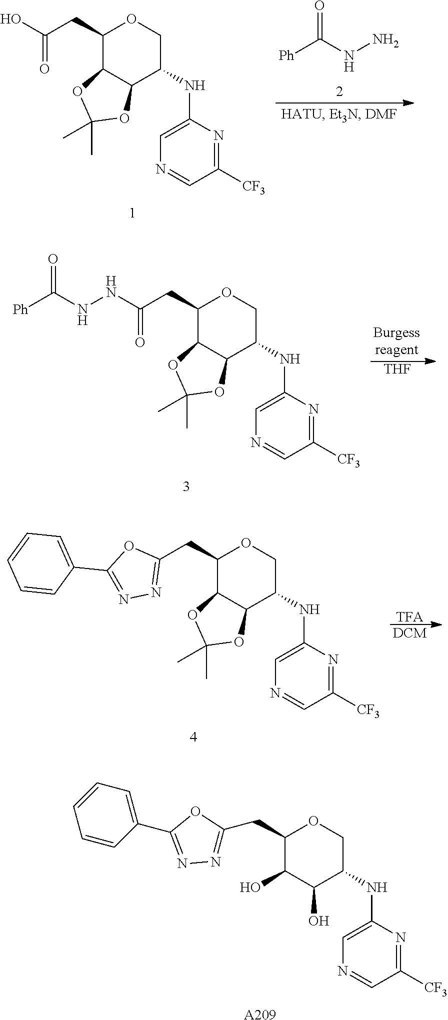

Step 1: To a solution of 1 (1.00 eq) in DMF (5 mL) was added HATU (1.5 eq) and Et₃N (3.0 eq), the solution was stirred at 25° C. for 20 min, then 2 (2.0 eq) was added, the resulting mixture was stirred at 25° C. for 16 hours. The mixture was diluted with water and extracted with EA. The organic layer was washed with brine, dried over Na₂SO₄, filtered and concentrated under reduced pressure. The crude product was purified by reverse phase (25˜60% MeOH in water) to give compound 3 as a yellow solid. Yield: 38%. LCMS found: [M+H]⁺=496.1.
Step 2: To a solution of 3 (1.00 eq) in THF (10 mL) was added Burgess reagent (3.0 eq). Then the reaction was stirred at 20° C. for 16 hours. The solution was quenched with water and extracted with EA. The organic layer was dried over Na₂SO₄, filtered and concentrated in vacuo. The residue was purified by reverse phase (30˜70% MeOH in water) to give compound 4 as a yellow solid. Yield: 70%. LCMS found: [M+H]⁺=478.1.
Step 3: To a solution of 4 (1.00 eq) in DCM (10 mL) was added TFA (5 eq), the mixture was stirred at 20° C. for 24 h. The mixture was concentrated in vacuo. The crude product was purified by reverse phase (25˜55% MeOH in water) to give A209 as a yellow solid. Yield: 26%. LCMS found: [M+H]⁺=438.3. ¹H NMR (400 MHz, CD₃OD) δ 8.11 (s, 1H), 8.06-8.02 (m, 2H), 8.00 (s, 1H), 7.92-7.89 (m, 1H), 7.53-7.50 (m, 2H), 4.41-4.32 (m, 1H), 4.13-4.02 (m, 2H), 3.96-3.94 (m, 1H), 3.74 (dd, J=10.4, 2.8 Hz, 1H), 3.49-3.47 (m, 1H), 3.43-3.37 (m, 1H), 3.14-3.12 (m, 1H).

### Preparation of (2R,3R,4S,5S)-2-((4-bromo-3-fluorophenoxy)methyl)-5-((6-(trifluoromethyl)pyrazin-2-yl)oxy)tetrahydro-2H-pyran-3,4-diol (A210)

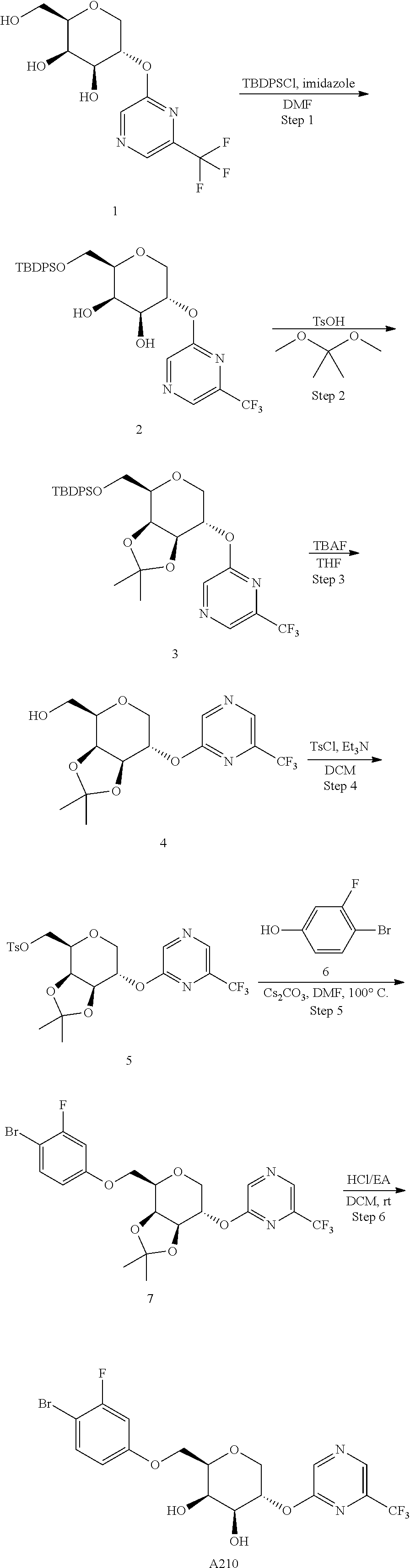

Step 1: To a solution of 1 (1.00 eq) in DMF (10 mL) was added imidazole (1.50 eq) slowly at 0° C., then TBDPS-Cl (1.50 eq) was added and the resulting mixture was stirred at 25° C. for 16 hours. TLC (PE/EA=1/1) showed the starting material was consumed completely, one major spot was detected. The reaction was quenched with saturated solution of NH₄Cl and extracted with EtOAc, the organic layer was dried over Na₂SO₄, filtered and concentrated under reduced pressure, the residue was purified by silica gel chromatography (PE/EA=5/1 to 1/1) to give 2 as colorless oil. Yield: 75%. LCMS found: [M+H]⁺=549.2
Step 2: To a solution of 2 (1.00 eq) in 2,2-dimethoxypropane (10 mL) was added TsOH·H₂O (0.10 eq), the reaction was stirred at 25° C. for 18 hours. The mixture was quenched with Et₃N and then concentrated in vacuo. The residue was purified by silica gel chromatography (PE/EA=10/1 to 3/1) to give 3 as colorless oil. Yield: 80%. LCMS found: [M+H]⁺=589.2
Step 3: To a solution of 3 (1.00 eq) in THF (50 mL) was added TBAF (1.50 eq), the reaction was stirred at 25° C. for 18 hours. TLC (PE/EA=1/1) showed the starting material was consumed completely, one major spot was detected. The reaction was concentrated under reduced pressure. The residue was purified by silica gel chromatography (PE/EA=10/1 to 2/1) to give 4 as colorless oil. Yield: 70%. LCMS found: [M+H]⁺=351.1
Step 4: To a solution of 4 (1.00 eq) in DCM (50 mL) was added ET₃N (2.00 eq) and TsCl (1.50 eq), the reaction was stirred at 25° C. for 12 hours. LCMS showed one major peak with desired mass was detected. The mixture was concentrated in vacuo, the residue was purified silica gel chromatography (PE/EA=10/1 to 3/1) to give 5 as colorless oil. Yield: 80%. LCMS found: [M+H]⁺=505.1
Step 5: To a solution of 5 (1.00 eq) and 6 (2.00 eq) in DMF (5 mL) was added Cs₂CO₃ (3.00 eq), the solution was stirred at 100° C. for 16 hours. The mixture was diluted with water and extracted with EtOAc. The organic layer was washed with brine, dried over Na₂SO₄, filtered and concentrated under reduced pressure. The residue was purified by reverse phase (70˜85% MeOH in water) to give compound 7 as a yellow solid. Yield: 24%. LCMS found: [M+H]⁺=523, 525.
Step 6: To a solution of 7 (1.00 eq) in DCM (5 mL) was added HCl/EtOAc (1 mL, 4M), the mixture was stirred at 25° C. for 16 hours. The solution was concentrated in vacuo. The crude product was purified by reverse phase (55˜65% MeOH in H₂O, 5% NH₃·H₂O) to give A210 as a white solid. Yield: 56%. LCMS found: [M+H]⁺=483.2, 485.2. ¹H NMR (400 MHz, CD₃OD) δ 8.56 (s, 1H), 8.50 (s, 1H), 7.48 (t, J=8.4 Hz, 1H), 6.87 (dd, J=10.8, 2.8 Hz, 1H), 6.75 (ddd, J=8.8, 2.8, 1.2 Hz, 1H), 5.42 (td, J=10.0, 5.2 Hz, 1H), 4.30 (dd, J=10.8, 5.2 Hz, 1H), 4.18 (d, J=5.6 Hz, 2H), 4.09 (d, J=3.2 Hz, 1H), 3.98-3.82 (m, 2H), 3.37 (d, J=10.4 Hz, 1H).

### Preparation of tert-butyl 4-(6-(((3S,4R,5R,6R)-4,5-dihydroxy-6-(hydroxymethyl)tetrahydro-2H-pyran-3-yl)amino)pyridin-2-yl)piperidine-1-carboxylate (A211)

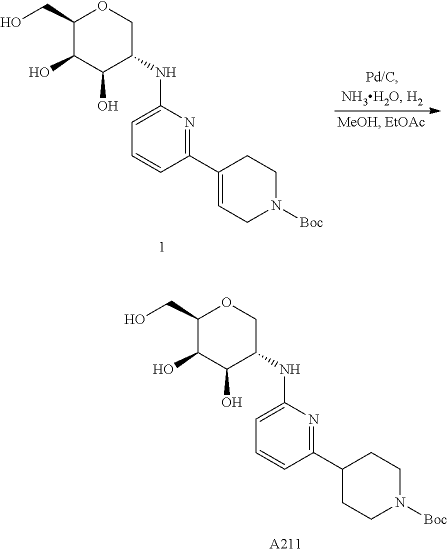 [SMILES: CC(C)(C)OC(=O)N1CC=C(c2cccc(N[C@H]3CO[C@H](CO)[C@H](O)[C@@H]3O)n2)CC1 ; CC(C)(C)OC(=O)N1CCC(c2cccc(N[C@H]3CO[C@H](CO)[C@H](O)[C@@H]3O)n2)CC1 ; "A211"]

To a solution of 1 (1.00 eq) in MeOH (10 mL) and EA (2 mL) was added Pd/C (10% purity) and NH₃·H₂O (1.00 eq), the mixture was degassed and purged with hydrogen for several times, then stirred at 20° C. for 1 h under hydrogen atmosphere (15 psi). The mixture was filtered, the filtrate was concentrated under reduced pressure, the residue was purified by reverse phase (5-35 MeCN in water, 0.1% TFA) to give A149 as a white solid. Yield: 11%. LCMS found: [M+H]⁺=424.3. ¹H NMR (400 MHz, CD₃OD) δ 7.41 (t, J=8.0 Hz, 1H), 6.53-6.40 (m, 2H), 4.23-4.06 (m, 4H) 3.89 (d, J=6.8 Hz, 1H), 3.76 (dd, J=11.2, 7.2 Hz, 1H), 3.69 (dd, J=11.2, 5.2 Hz, 1H), 3.58 (dd, J=9.6, 3.2 Hz, 1H), 3.49-3.39 (m, 1H), 3.19 (t, J=12.4 Hz, 1H), 2.93-2.77 (m, 2H), 2.71 (tt, J=12.0, 3.2 Hz, 1H), 1.91-1.81 (m, 2H), 1.73-1.57 (m, 2H), 1.47 (s, 9H).

### Preparation of (2R,3R,4R,5S)-2-((pyridazin-3-yloxy)methyl)-5-((6-(trifluoromethyl)pyrazin-2-yl)amino)tetrahydro-2H-pyran-3,4-diol (A212)

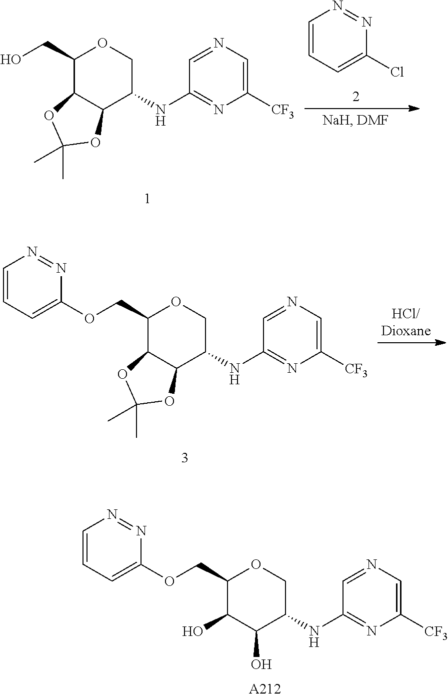

Step 1: To a solution of 1 (1.00 eq) and 2 (2.00 eq) in DMF (2 mL) was added NaH (3.00 eq, 60% purity) at 0° C. The reaction was stirred at 25° C. for 12 hours under N₂ atmosphere. LCMS showed the desired mass was detected. The mixture was diluted with water and extracted with EtOAc, the organic layer was washed with brine, dried over Na₂SO₄, filtered and concentrated under reduced pressure. The crude product was purified by flash chromatography (silica gel, 0˜90% EA in PE) to give 3 as a yellow oil. Yield: 81.7%. LC-MS (ESI) found: 428 [M+H]⁺.
Step 2: The solution of 3 (1.00 eq) in HCl/1,4-dioxane (2 mL) was stirred at 25° C. for 1 hour. LCMS showed the desired mass was detected. The mixture was concentrated in vacuo. The crude product was purified by prep-HPLC to give A212 as a yellow solid. Yield: 75.0%. LCMS found: [M+H]⁺=388. ¹H NMR (400 MHz, CD₃OD): δ 9.07 (dd, J=4.8, 0.8 Hz, 1H), 8.13 (s, 1H), 8.01 (s, 1H), 7.98 (dd, J=8.8, 4.8 Hz, 1H), 7.65 (dd, J=8.8, 0.8 Hz, 1H), 4.73-4.65 (m, 2H), 4.38 (td, J=10.6, 5.1 Hz, 1H), 4.17 (dd, J=11.0, 5.1 Hz, 1H), 4.04 (d, J=3.1 Hz, 1H), 3.97-3.93 (m, 1H), 3.73 (dd, J=10.6, 3.2 Hz, 1H), 3.19 (t, J=10.8 Hz, 1H).

### Preparation of tert-butyl 4-(4-(((2R,3R,4S,5S)-3,4-dihydroxy-5-((6-(trifluoromethyl)pyridin-2-yl)oxy)tetrahydro-2H-pyran-2-yl)methoxy)-2-fluorophenyl) piperazine-1-carboxylate (A213)

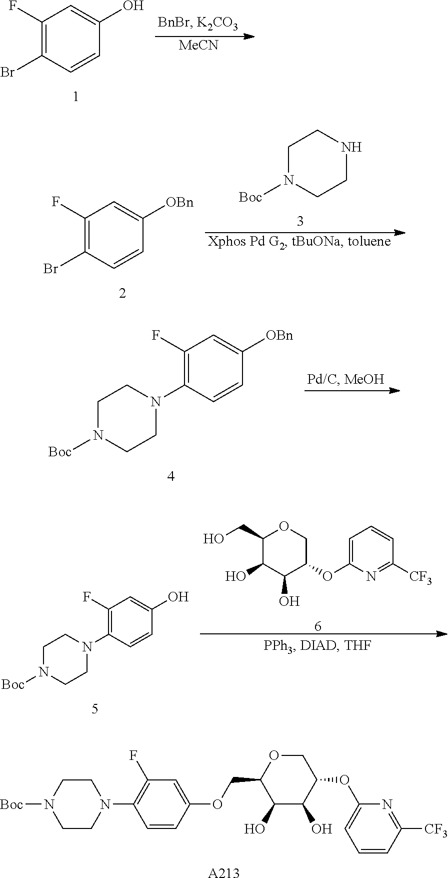

Step 1: To a solution of 1 (1.00 eq) and K₂CO₃ (2.50 eq) in CH₃CN (20 mL) was added BnBr (1.20 eq), the reaction was stirred at 60° C. for 8 hours. The mixture was filtered, the filtrate was concentrated under reduced pressure, the residue was purified by flash chromatography (silica gel, 0˜50% EA in PE) to give 2 as a colorless oil. Yield: 70%.
Step 2: To a solution of 2 (1.00 eq) and 3 (1.20 eq) in toluene (5 mL) were added t-BuONa (2.20 eq) and XPhos Pd G2 (0.10 eq), the reaction was degassed and purged with nitrogen for several times, then stirred at 90° C. for 3 hours under nitrogen atmosphere. The mixture was concentrated in vacuo. The residue was purified by flash chromatography (silica gel, 0˜70% EA in PE) to give 4 as a yellow oil. Yield: 50%. LC-MS (ESI) found: 387 [M+H]⁺.
Step 3: To a solution of 4 (1.00 eq) in MeOH (10 mL) was added Pd/C (10% purity), the mixture was degassed and purged with hydrogen for several times, then stirred at 25° C. for 3 hours under hydrogen atmosphere (15 psi). The mixture was filtered through a Celite pad, and the filtrate was concentrated to give crude product. The residue was purified by reverse phase (C18, 5-70% MeCN in water) to afford 5 as white solid. Yield: 52.3%. LC-MS (ESI) found: 297 [M+H]⁺.
Step 4: To a solution of 5 (1.00 eq), 6 (1.20 eq) and PPh₃ (4.00 eq) in THF (3 mL) was added DIAD (4.00 eq) at 0° C. The mixture was stirred at 60° C. for 3 hours under nitrogen atmosphere. The mixture was concentrated under reduced pressure. The residue was purified by reverse phase (C18, 5-70% MeCN in water, 0.1% TFA) to afford A213 as white solid. Yield: 1.1%. LCMS found: [M+H]⁺=588. ¹H NMR (400 MHz, CD₃OD): δ 7.87 (t, J=8.0 Hz, 1H), 7.36 (d, J=7.6 Hz, 1H), 7.05 (d, J=8.4 Hz, 1H), 6.99 (t, J=8.8 Hz, 1H), 6.81-6.53 (m, 2H), 5.38 (td, J=10.0, 5.2 Hz, 1H), 4.37 (dd, J=10.8, 5.2 Hz, 1H), 4.18-4.11 (m, 2H), 4.07 (d, J=2.8 Hz, 1H) 3.92-3.81 (m, 2H), 3.59-3.52 (m, 4H), 3.27 (t, J=10.8 Hz, 1H), 2.96-2.88 (m, 4H), 1.47 (s, 9H).

### Preparation of (2R,3R,4S,5R,6R)-6-ethynyl-2-methyl-5-((6-(trifluoromethyl)pyridin-2-yl)oxy)tetrahydro-2H-pyran-3,4-diol (A214)

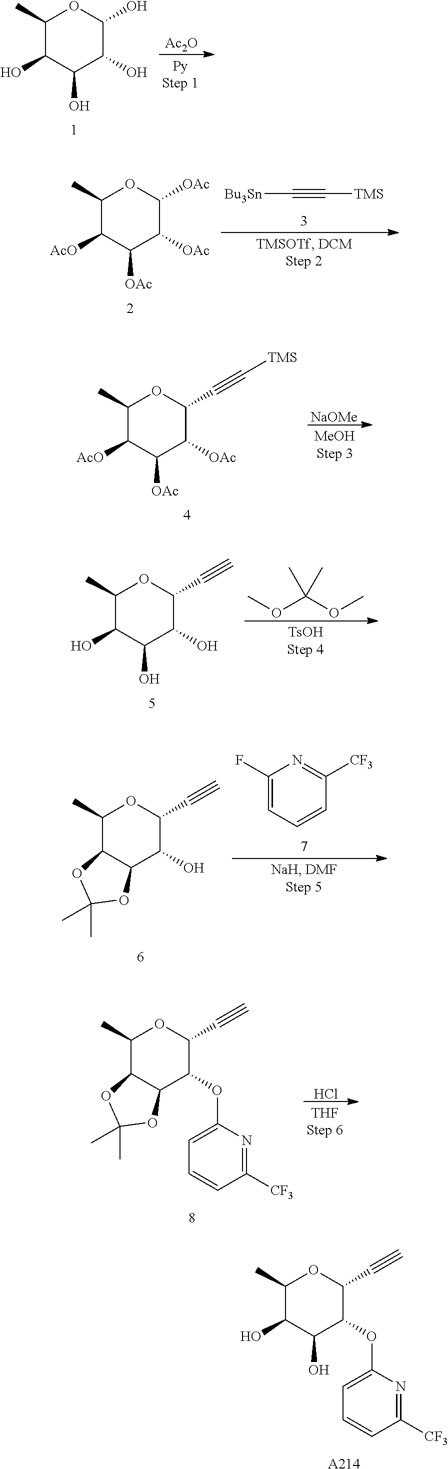

Step 1: To a solution of 1 (1.00 eq) in pyridine (5 mL) was added Ac₂O (10.00 eq) dropwise at 0° C. The reaction was stirred at 25° C. for 18 hours. LCMS showed the desired mass was detected. The resulting mixture was concentrated in vacuo. The crude product was purified by flash chromatography (silica gel, 0˜40% EA in PE) to give 2 as a colorless oil. Yield: 61.7%. LC-MS (ESI) found: 333 [M+H]⁺. ¹H NMR (400 MHz, CDCl₃): δ 6.33 (d, J=2.8 Hz, 1H), 5.35-5.31 (m, 3H), 4.27 (q, J=6.5 Hz, 1H), 2.17 (s, 3H), 2.14 (s, 3H), 2.00 (d, J=4.8 Hz, 6H), 1.15 (d, J=6.5 Hz, 3H).
Step 2: To a solution of 2 (1.00 eq) and 3 (3.00 eq) in DCM (1 mL) was added TMSOTf (3.00 eq) at 0° C. The reaction was stirred at 25° C. for 3 hours. The reaction was quenched with TEA. The reaction was diluted with DCM (30 mL), washed with H₂O (10 mL*2) and brine (10 mL), the organic layer was separated, dried over Na₂SO₄ and concentrated in vacuo. The crude product was purified by flash chromatography (silica gel, 0˜10% EA in PE) to give 4 as a colorless oil. Yield: 22.8%. LC-MS (ESI) found: 393 [M+Na]⁺. ¹H NMR (400 MHz, CDCl₃): δ 5.35-5.27 (m, 2H) 5.12 (ddd, J=12.2, 5.9, 1.9 Hz, 1H), 5.03 (d, J=5.9 Hz, 1H), 4.29 (q, J=6.4 Hz, 1H), 2.15 (s, 3H), 2.07 (s, 3H), 2.00 (s, 3H), 1.16 (d, J=6.5 Hz, 3H), 0.27-0.14 (m, 9H).
Step 3: To a solution of 4 (1.00 eq) in MeOH (3 mL) was added NaOMe (0.30 eq) at 0° C. The mixture was stirred at 25° C. for 1.5 hours. LCMS showed the desired mass was detected. The resulting mixture was neutralized by the addition of AMBERLITE IR-120. The resulting mixture was filtered and the filtrate was concentrated in vacuo to afford 5 (crude) as a colorless oil. Yield: 92.2%. LC-MS (ESI) found: 173 [M+H]⁺. ¹H NMR (400 MHz, CD₃OD): δ 4.63 (dd, J=5.9, 2.3 Hz, 1H), 4.11 (qd, J=6.5, 1.0 Hz, 1H), 3.82 (dd, J=10.0, 5.9 Hz, 1H), 3.72 (dd, J=10.0, 3.3 Hz, 1H), 3.65 (dd, J=3.3, 1.1 Hz, 1H), 2.89 (d, J=2.3 Hz, 1H), 1.21 (d, J=6.5 Hz, 3H).
Step 4: To a solution of 5 (1.00 eq) in 2,2-dimethoxypropane (3 mL) was added TsOH (0.20 eq) at 25° C. The mixture was stirred for 3 hours. The reaction was quenched with Et₃N. The reaction was diluted with EA (50 mL), washed with H₂O (20 mL*2) and brine (10 mL). The organic layer was separated, dried over Na₂SO₄ and concentrated in vacuo. The crude product was purified by flash chromatography (silica gel, 0˜50% EA in PE) to give 6 as a colorless oil. Yield: 56.0%. LC-MS (ESI) found: 213 [M+H]⁺. ¹H NMR (400 MHz, CDCl₃): δ 4.71 (dd, J=5.4, 2.3 Hz, 1H), 4.36 (qd, J=6.6, 2.3 Hz, 1H), 4.17 (dd, J=7.1, 5.8 Hz, 1H), 4.06 (dd, J=5.7, 2.3 Hz, 1H), 3.82 (dd, J=7.2, 5.5 Hz, 1H), 2.57 (d, J=2.3 Hz, 1H), 1.52 (s, 3H), 1.36 (s, 3H).
Step 5: To a solution of 6 (1.00 eq) and 7 (3.00 eq) in DMF (3 mL) was added NaH (3.00 eq, 60% purity) at 0° C. The mixture was stirred at 25° C. for 1 hour. The resulting mixture was quenched with saturated solution of NH₄Cl, then extracted with EA (50 mL), the organic layer was washed with H₂O (20 mL*2) and brine (20 mL*2), dried over Na₂SO₄, filtered and concentrated in vacuo. The crude product was purified by flash chromatography (silica gel, 0˜20% EA in PE) to give 8 as a colorless oil. Yield: 47.5%. LC-MS (ESI) found: 358 [M+H]⁺. ¹H NMR (400 MHz, CDCl₃): δ 7.77-7.69 (m, 1H), 7.28 (d, J=7.3 Hz, 1H), 7.00 (d, J=8.4 Hz, 1H), 5.34 (dd, J=8.2, 5.8 Hz, 1H), 5.15 (dd, J=5.7, 2.3 Hz, 1H), 4.47 (ddd, J=6.6, 4.9, 2.4 Hz, 2H), 4.16 (dd, J=5.3, 2.4 Hz, 1H), 2.49 (d, J=2.4 Hz, 1H), 1.54 (s, 3H), 1.44 (d, J=6.6 Hz, 3H), 1.37 (s, 3H).
Step 6: To a solution of 8 (1.00 eq) in THF (1 mL) was added HCl (1 mL). The reaction was stirred at 25° C. for 2 hours. The mixture was concentrated in vacuo. The crude product was purified by prep-HPLC to give A214 as a white solid. Yield: 70.4%. LC-MS (ESI) found: 318 [M+H]⁺. ¹H NMR (400 MHz, CD₃OD): δ 7.89 (d, J=8.0 Hz, 1H), 7.39 (d, J=8.0 Hz, 1H), 7.09 (d, J=8.0 Hz, 1H), 5.28-5.22 (m, 2H), 4.21 (qd, J=6.4, 1.2 Hz, 1H), 4.10 (dq, J=8.0, 3.2 Hz, 1H), 3.78 (dd, J=3.4, 1.2 Hz, 1H), 2.92 (d, J=2.0 Hz, 1H), 1.27 (d, J=6.4 Hz, 3H). ¹⁹F NMR (377 MHz, CD₃OD): δ −69.90 (s).

### Preparation of 4′-(((2R,3R,4R,5S)-3,4-dihydroxy-5-((6-(trifluoromethyl)pyrazin-2-yl)amino) tetrahydro-2H-pyran-2-yl)methoxy)-[1,1′-biphenyl]-4-carboxylic acid (A215)

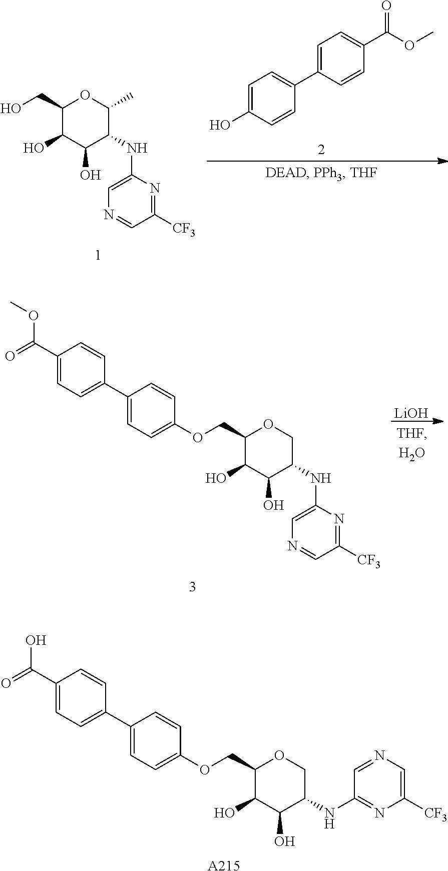

Step 1: To a solution of 1 (1.00 eq) and 2 (1.50 eq) in THF (3 mL) was added PPh₃ (3.00 eq) and DEAD (3.00 eq) at 25° C. The reaction was stirred at 70° C. for 12 hours under N₂ atmosphere. LCMS showed the desired mass was detected. The mixture was diluted with EtOAc, washed with H₂O and brine, the organic layer was separated, dried over Na₂SO₄, filtered and concentrated in vacuo. The crude product was purified by flash chromatography (silica gel, 0˜90% EA in PE) to give 3 as a yellow oil. Yield: 39.7%. LC-MS (ESI) found: 520 [M+H]⁺.
Step 2: To a solution of 3 (1.00 eq) in THF (3 mL) and H₂O (2 mL) was added LiOH (2.00 eq) at 0° C. The reaction was stirred at 25° C. for 12 hours. LCMS showed the desired mass was detected. The pH of the mixture was adjusted to 6 with HCl (1N), the mixture was concentrated under reduced pressure. The crude product was purified by prep-HPLC to give A215 as a white solid. Yield: 29.7%. LC-MS (ESI) found: 506 [M+H]⁺. ¹H NMR (400 MHz, DMSO) δ 12.88 (brs, 1H) 8.24 (s, 1H), 8.08 (s, 1H), 7.99 (d, J=8.4 Hz, 2H), 7.75 (d, J=8.4 Hz, 2H), 7.71-7.65 (m, 3H), 7.07 (d, J=8.4 Hz, 2H), 4.96 (brs, 2H), 4.20-4.08 (m, 3H), 3.98-3.85 (m, 2H), 3.81-3.74 (m, 1H), 3.60 (d, J=9.8 Hz, 1H), 3.05 (t, J=10.6 Hz, 1H).

### Preparation of (2R,3R,4S,5S)-2-((pyridazin-3-yloxy)methyl)-5-((6-(trifluoromethyl)pyridin-2-yl)oxy)tetrahydro-2H-pyran-3,4-diol (A216)

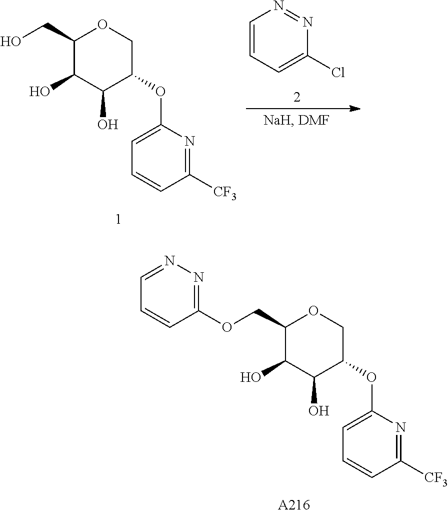 [SMILES: OC[C@H]1OC[C@H](Oc2cccc(C(F)(F)F)n2)[C@@H](O)[C@H]1O ; Clc1cccnn1 ; O[C@@H]1[C@H](O)[C@@H](Oc2cccc(C(F)(F)F)n2)CO[C@@H]1COc1cccnn1 ; "A216"]

Step 1: To a solution of 1 (1.00 eq) and 2 (1.00 eq) in DMF (2 mL) was added NaH (3.00 eq, 60% purity) at 0° C. The reaction was stirred at 25° C. for 12 hours under N₂ atmosphere. LCMS showed the desired mass was detected. The mixture was quenched with saturated solution of NH₄Cl, extracted with EtOAc, the organic layer was washed with brine, dried with anhydrous Na₂SO₄, filtered and concentrated in vacuo. The crude product was purified by prep-HPLC to give A154 as a white solid. Yield: 36.7%. LCMS found: [M+H]⁺=388. ¹H NMR (400 MHz, CD₃OD): δ 9.35 (dd, J=5.2, 0.8 Hz, 1H), 8.40 (dd, J=9.2, 5.2 Hz, 1H), 8.12 (dd, J=9.2, 0.8 Hz, 1H), 7.88 (t, J=8.0 Hz, 1H), 7.38 (d, J=8.0 Hz, 1H), 7.06 (d, J=8.0 Hz, 1H), 5.38 (td, J=10.0, 5.2 Hz, 1H), 4.68 (dd, J=11.2, 4.0 Hz, 1H), 4.38 (dd, J=10.8, 5.2 Hz, 1H), 4.11 (dd, J=3.6, 1.2 Hz, 1H), 4.03 (ddd, J=8.0, 3.6, 1.2 Hz, 1H), 3.92 (dd, J=10.0, 3.6 Hz, 1H), 3.30 (t, J=10.8 Hz, 1H).

### Preparation of tert-butyl 4-(4-((2R,3R,4S,5R,6R)-4,5-dihydroxy-6-methyl-3-((6-(trifluoromethyl)pyridin-2-yl)oxy)tetrahydro-2H-pyran-2-yl)-1H-1,2,3-triazol-1-yl)piperidine-1-carboxylate (A217)

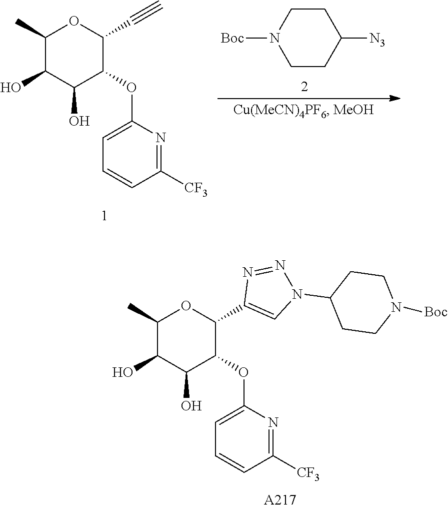

Step 1: To a solution of 1 (1.00 eq) and 2 (2.00 eq) in MeOH (1 mL) was added Cu(MeCN)₄PF₆ (1.00 eq) at 0° C. The reaction was stirred at 25° C. for 3 hours under nitrogen atmosphere. LCMS showed the desired mass was detected. The resulting mixture was concentrated in vacuo. The crude product was purified by prep-HPLC to give A217 as a white solid. Yield: 35.0%. LC-MS (ESI) found: 544 [M+H]⁺. ¹H NMR (400 MHz, CD₃OD): δ 8.03 (s, 1H), 7.81 (t, J=8.0 Hz, 1H), 7.34 (d, J=8.0 Hz, 1H), 6.94 (d, J=8.0 Hz, 1H), 5.72 (d, J=6.4 Hz, 1H), 5.63 (dd, J=10.0, 6.4 Hz, 1H), 4.66 (tt, J=11.6, 4.2 Hz, 1H), 4.48 (dd, J=10.0, 3.6 Hz, 1H), 4.22-4.13 (m, 2H), 3.97 (qd, J=6.0, 1.6 Hz, 1H), 3.85 (dd, J=3.6, 1.2 Hz, 1H), 3.06-2.91 (m, 2H), 2.14-2.06 (m, 2H), 1.99-1.87 (m, 2H), 1.47 (s, 9H), 1.24 (d, J=6.4 Hz, 3H). ¹⁹F NMR (377 MHz, CD₃OD): δ −69.79 (s).

### Preparation of (2R,3R,4S,5R,6R)-2-methyl-6-(1-(piperidin-4-yl)-1H-1,2,3-triazol-4-yl)-5-((6-(trifluoromethyl)pyridin-2-yl)oxy)tetrahydro-2H-pyran-3,4-diol (A218)

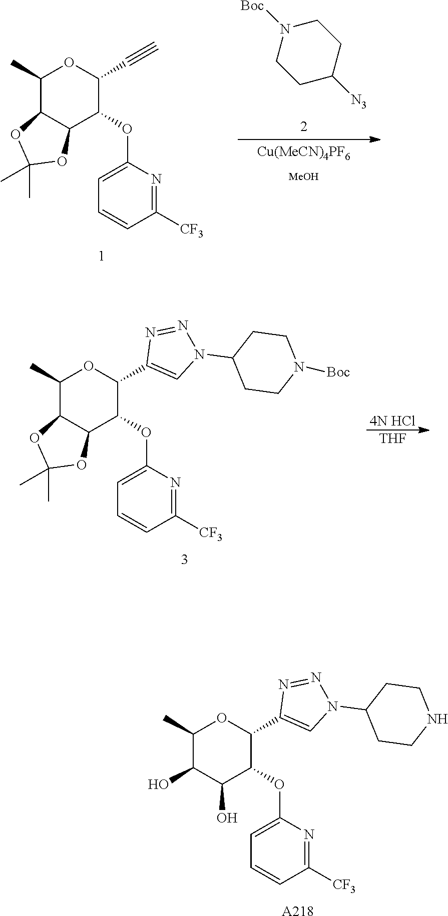

Step 1: To a solution of 1 (1.00 eq) and 2 (2.00 eq) in MeOH (1 mL) was added Cu(MeCN)₄PF₆ (1.00 eq) at 0° C. The reaction was stirred at 25° C. for 3 hours under nitrogen atmosphere. LCMS showed the desired mass was detected. The resulting mixture was concentrated in vacuo. The crude product was purified by reverse phase (C18, 5-50% Acetonitrile in water, 0.1% TFA) to give 3 as a white solid. Yield: 35.0%. LC-MS (ESI) found: 584 [M+H]⁺.
Step 2: A solution of 3 (1.00 eq) in THF (1 mL) was added 4N HCl (1 mL). The reaction was stirred at 25° C. for 2 hours. The mixture was concentrated in vacuo. The residue was purified by prep-HPLC to give A218 as a white solid. Yield: 54.8%. LC-MS (ESI) found: 444 [M+H]⁺. ¹H NMR (400 MHz, CD₃OD): δ 8.04 (s, 1H), 7.82 (t, J=8.0 Hz, 1H), 7.35 (d, J=8.0 Hz, 1H), 6.94 (d, J=8.0 Hz, 1H), 5.75 (d, J=6.4 Hz, 1H), 5.62 (dd, J=10.0, 6.4 Hz, 1H), 4.83-4.77 (m, 1H) 4.50 (dd, J=10.0, 3.6 Hz, 1H), 3.99 (qd, J=6.4, 1.2 Hz, 1H), 3.86 (dd, J=3.6, 1.6 Hz, 1H), 3.59-3.52 (m, 2H), 3.26-3.19 (m, 2H), 2.43-2.25 (m, 4H), 1.24 (d, J=6.4 Hz, 3H). ¹⁹F NMR (377 MHz, CD₃OD): δ −69.80 (s).

### Preparation of (2R,3R,4S,5S)-2-ethynyl-5-((6-(trifluoromethyl)pyridin-2-yl)oxy)tetrahydro-2H-pyran-3,4-diol (A219)

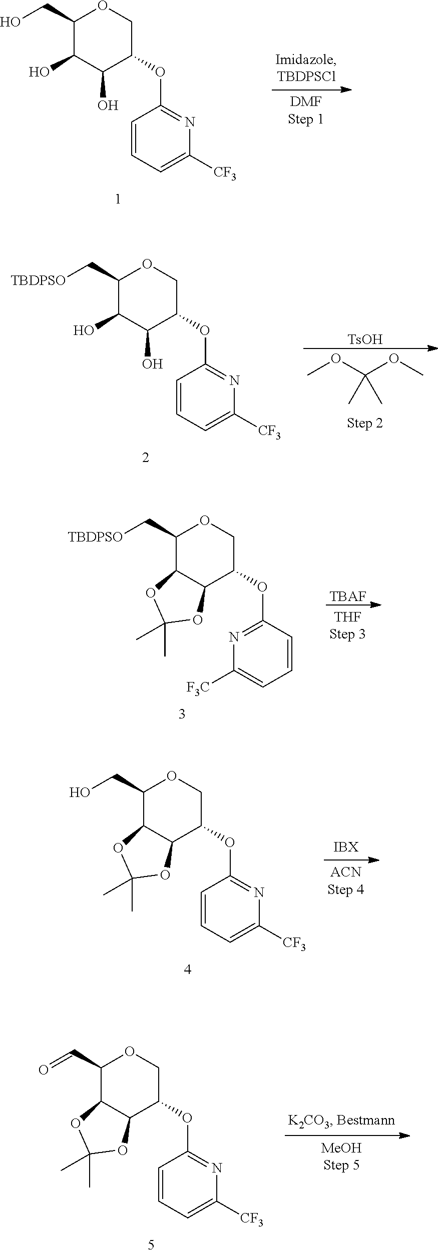

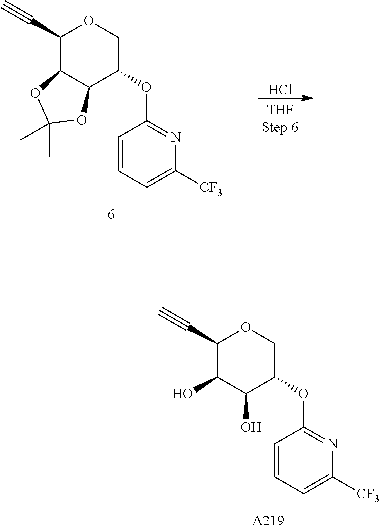 [SMILES: C#C[C@H]1OC[C@H](Oc2cccc(C(F)(F)F)n2)[C@H]2OC(C)(C)O[C@H]21 ; Cl ; C#C[C@H]1OC[C@H](Oc2cccc(C(F)(F)F)n2)[C@@H](O)[C@H]1O ; "A219"]

Step 1: To a solution of 1 (1.00 eq) and imidazole (3.00 eq) in DMF (25 mL) was added TBDPS-Cl (1.50 eq) at 0° C. The reaction was stirred at 25° C. for 12 hours. The mixture was diluted with water, extracted with EtOAc, the organic layer was concentrated and the residue was purified by flash (PE/EA=10/1 to 1:1) to afford 2 as a colorless oil. Yield: 72.60%. LC-MS (ESI) found: 570 [M+Na]⁺.
Step 2: To a solution of 2 (1.00 eq) in 2,2-dimethoxypropane (30 mL) was added TsOH (0.10 eq), the mixture was stirred at 80° C. for 4 hours. The mixture was quenched with Et₃N, then concentrated in vacuo. The residue was purified by flash (PE/EA=10/1 to 3:1) to afford 3 as a colorless oil. Yield: 82.83%. LC-MS (ESI) found: 610 [M+Na]⁺.
Step 3: The solution of 3 (1.00 eq) and TBAF (2.00 eq) in THF (25 mL) was stirred at 20° C. for 4 hours. The mixture was concentrated and the residue was purified by flash (PE/EA=5/1 to 1:1) to afford 4 as a colorless oil. Yield: 77.11%. LC-MS (ESI) found: 350 [M+H]⁺.
Step 4: The solution of 4 (1.00 eq) and IBX (2.00 eq) in CH₃CN (2 mL) was stirred at 80° C. for 1 hour. The resulting mixture was filtered, the filtrate was diluted with water, then extracted with EtOAc, the organic layer was dried over anhydrous sodium sulfate, filtered and concentrated in vacuo to give crude 5 as a colorless oil. Yield: 73.82%. LC-MS (ESI) found: 348 [M+H]⁺.
Step 5: To a solution of 5 (1.00 eq) and K₂CO₃ (2.00 eq) in MeOH (15 mL) was added Bestmann-Ohira reagent (1.20 eq) dropwise at 0° C. The reaction was stirred at 25° C. for 3 hours. The mixture was concentrated and the residue was diluted with water, extracted with EtOAc, the organic layer was concentrated and the residue was purified by flash chromatography (silica gel, 0˜70% EA in PE) to give 6 as a yellow oil. Yield: 15.81%. LC-MS (ESI) found: 344 [M+H]⁺.
Step 6: To a solution of 6 (1.00 eq) in THF (1 mL) was added HCl (0.3 mL, 1N), the mixture was stirred at 25° C. for 1 hour. The mixture was concentrated under reduced pressure, the residue was purified by prep-HPLC to give A219 as a white solid. Yield: 7.55%. LCMS found: [M+H]⁺=304. ¹H NMR (400 MHz, CD₃OD): δ 7.88 (t, J=8.0 Hz, 1H), 7.38 (d, J=7.6 Hz, 1H), 7.06 (d, J=8.4 Hz, 1H), 5.34 (td, J=9.6, 5.2 Hz, 1H), 4.39 (t, J=2.0 Hz, 1H), 4.32 (dd, J=11.2, 5.2 Hz, 1H) 4.00 (dd, J=3.6, 1.6 Hz, 1H), 3.88 (dd, J=9.2, 3.6 Hz, 1H), 3.30-3.26 (m, 1H), 2.91 (d, J=2.0 Hz, 1H).

### Preparation of tert-butyl 4-(4-((2R,3R,4S,5S)-3,4-dihydroxy-5-((6-(trifluoromethyl)pyridin-2-yl)oxy)tetrahydro-2H-pyran-2-yl)-1H-1,2,3-triazol-1-yl)piperidine-1-carboxylate (A220)

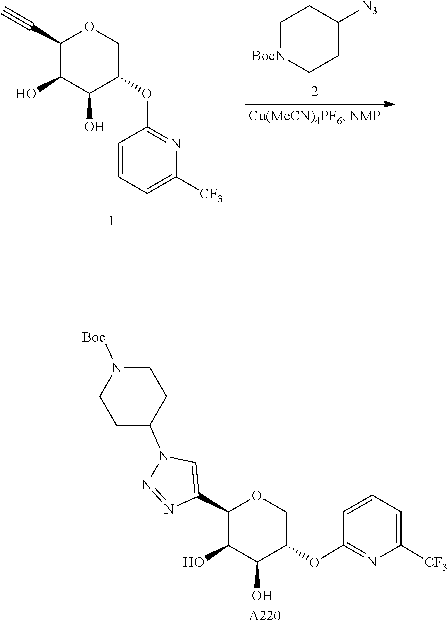

The solution of 1 (1.00 eq), 2 (1.50 eq) and Cu(MeCN)₄PF₆ (2.00 eq) in NMP (1 mL) was stirred at 25° C. for 1 hour under nitrogen atmosphere. The mixture was directly purified by prep-HPLC to give A220 as a white solid. Yield: 11.45%. LCMS found: [M+H]⁺=530. ¹H NMR (400 MHz, CD₃OD): δ 8.02 (s, 1H), 7.88 (t, J=8.0 Hz, 1H), 7.37 (d, J=7.6 Hz, 1H), 7.07 (d, J=8.4 Hz, 1H), 5.44 (dd, J=10.0, 5.2 Hz, 1H), 4.79 (s, 1H), 4.75-4.66 (m, 1H), 4.43 (dd, J=10.8, 5.6 Hz, 1H), 4.21 (d, J=13.8 Hz, 2H), 4.15 (d, J=2.8 Hz, 1H), 4.03 (dd, J=10.0, 3.2 Hz, 1H), 3.40 (t, J 10=10.8 Hz, 1H), 3.08-2.94 (m, 2H), 2.21-2.13 (m, 1H), 2.03-1.93 (m, 2H), 1.48 (s, 9H).

### Preparation of 6-(4-(((2R,3R,4S,5S)-3,4-dihydroxy-5-((6-(trifluoromethyl)pyridin-2-yl)oxy)tetrahydro-2H-pyran-2-yl)methoxy)phenyl)pyridazine-3-carboxylic acid (A221)

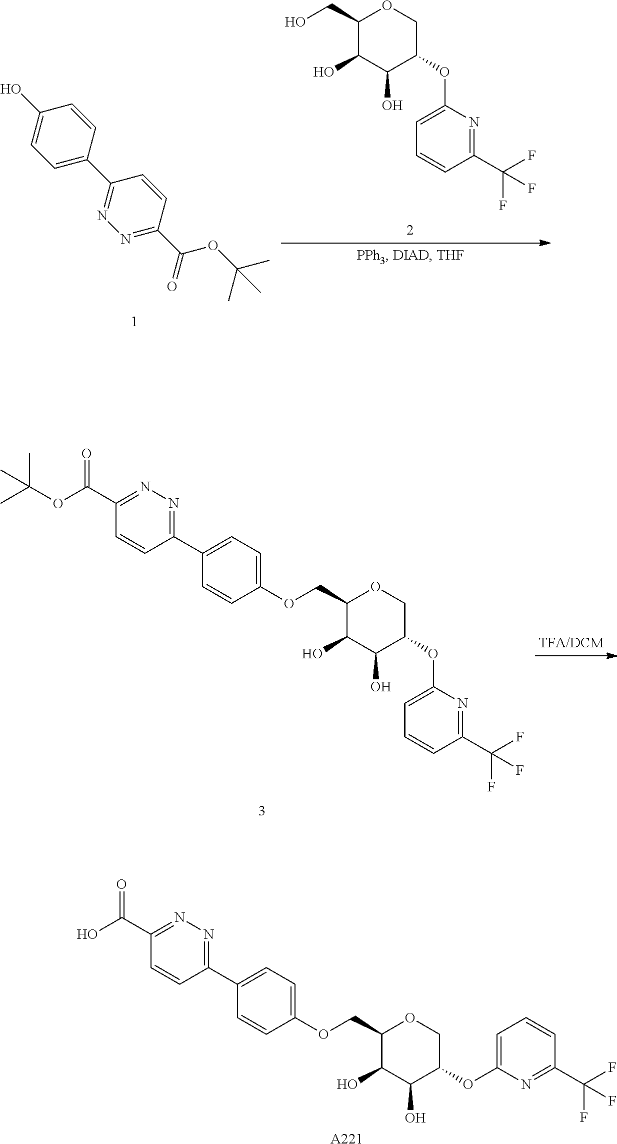 [SMILES: CC(C)(C)OC(=O)c1ccc(-c2ccc(O)cc2)nn1 ; OC[C@H]1OC[C@H](Oc2cccc(C(F)(F)F)n2)[C@@H](O)[C@H]1O ; CC(C)(C)OC(=O)c1ccc(-c2ccc(OC[C@H]3OC[C@H](Oc4cccc(C(F)(F)F)n4)[C@@H](O)[C@H]3O)cc2)nn1 ; O=C(O)c1ccc(-c2ccc(OC[C@H]3OC[C@H](Oc4cccc(C(F)(F)F)n4)[C@@H](O)[C@H]3O)cc2)nn1 ; "A221"]

Step 1: To a solution of 1 (1.00 eq), 2 (1.20 eq) and PPh3 (3.00 eq) in THF (20 mL) was added DIAD (3.00 eq) at 0° C. The reaction was stirred at 60° C. for 2 hours under nitrogen atmosphere. LCMS showed the desired mass was detected. The resulting mixture was filtered and the filtrate was concentrated in vacuo to give crude 3 as a colorless oil. LC-MS (ESI) found: 564 [M+H]⁺.
Step 2: To a solution of 3 (1.00 eq) in DCM (2 mL) was added TFA (1 mL), the mixture was stirred at 25° C. for 2 hours. LCMS showed the desired mass was detected. The mixture was concentrated in vacuo. The residue was purified by reverse phase (5-50% MeCN in Water, 0.1% TFA) to give A221 as a white solid. Yield: 63.46%. LCMS found: [M+H]⁺=508. ¹H NMR (400 MHz, CD₃OD) δ 8.32-8.26 (m, 2H), 8.18 (t, J=8.8 Hz, 2H), 7.87 (t, J=8.0 Hz, 1H), 7.37 (d, J=7.6 Hz, 1H), 7.16 (t, J=8.8 Hz, 2H), 7.06 (d, J=8.4 Hz, 1H), 5.41 (td, J=10.0, 5.2 Hz, 1H) 4.40 (dd, J=10.8, 5.2 Hz, 1H), 4.29 (d, J=6.0 Hz, 2H), 4.13 (dd, J=3.6, 0.8 Hz, 1H), 3.97-3.88 (m, 2H), 3.32 (t, J=10.8 Hz, 1H).

### Preparation of (2R,3R,4R,5S)-2-(((5-(hydroxymethyl)pyrimidin-2-yl)oxy)methyl)-5-((6-(trifluoromethyl)pyrazin-2-yl)amino)tetrahydro-2H-pyran-3,4-diol (A222)

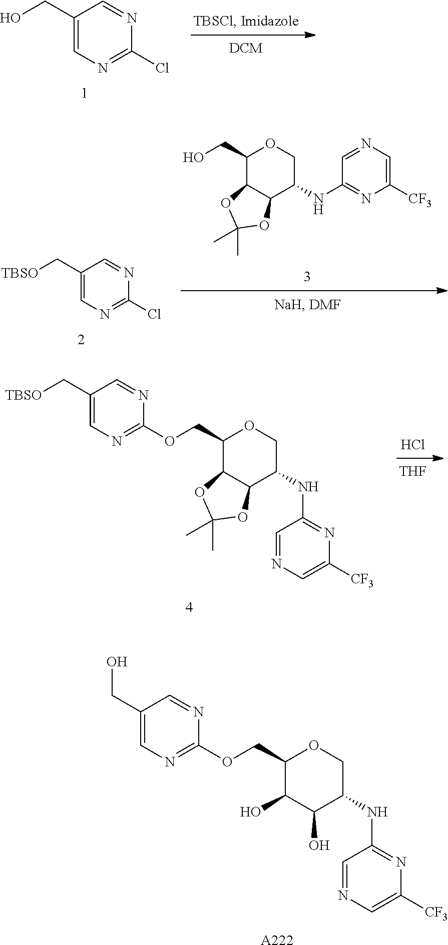

Step 1: To a solution of 1 (1.00 eq) and imidazole (1.50 eq) in DCM was added TBS-Cl (1.20 eq) at 25° C. The reaction mixture was stirred at 25° C. for 16 hours. TLC (PE/EA=10/1) showed the reaction was completed. The mixture was diluted with water, extracted with DCM, the organic layer was washed with water and brine, dried over anhydrous sodium sulfate, filtered and concentrated under reduced pressure. The residue was purified by silica gel chromatography (PE:EA=10:1) to give 2 as a yellow oil. Yield: 37.24%. LC-MS (ESI) found: [M+H]⁺=259.
Step 2: To a solution of 2 (1.00 eq) in DMF was added 3 (1.10 eq) and NaH (3.00 eq, 60% purity) at 0° C. The reaction mixture was stirred at 25° C. for 16 hours. LCMS showed the reaction was completed. The mixture was quenched with saturated solution of NH₄Cl, extracted with ethyl acetate, the organic layer was dried over anhydrous sodium sulfate, filtered and concentrated under reduced pressure, the residue was purified by silica gel chromatography (PE:EA=1:1) to give 4 as a yellow oil. Yield: 48.44%. LC-MS (ESI) found: [M+H]⁺=572.
Step 3: To a solution of 4 (1.00 eq) in THE was added HCl (1 mL, 2 N) at 25° C. The reaction mixture was stirred at 25° C. for 2 hours. LCMS showed the reaction was completed. The mixture was concentrated under reduced pressure and the residue was purified by prep-HPLC to afford A222 as a white solid. Yield: 20.55%. LC-MS (ESI) found: [M+H]⁺=418. ¹H NMR (400 MHz, CD₃OD) δ 8.55 (s, 2H), 8.11 (s, 1H), 8.00 (s, 1H), 4.62-4.50 (m, 4H), 4.37 (td, J=10.8, 5.2 Hz, 1H), 4.15 (dd, J=11.2, 5.2 Hz, 1H), 4.03 (d, J=2.4 Hz, 1H), 3.86 (ddd, J=6.4, 5.2, 0.8 Hz, 1H) 3.69 (dd, J=10.4, 3.2 Hz, 1H), 3.16 (t, J=10.8 Hz, 1H).

### Preparation of 4-(4-(((2R,3R,4R,5S)-3,4-dihydroxy-5-((6-(trifluoromethyl) pyrazin-2-yl) amino) tetrahydro-2H-pyran-2-yl) methyl) piperazin-1-yl) benzoic acid (A223)

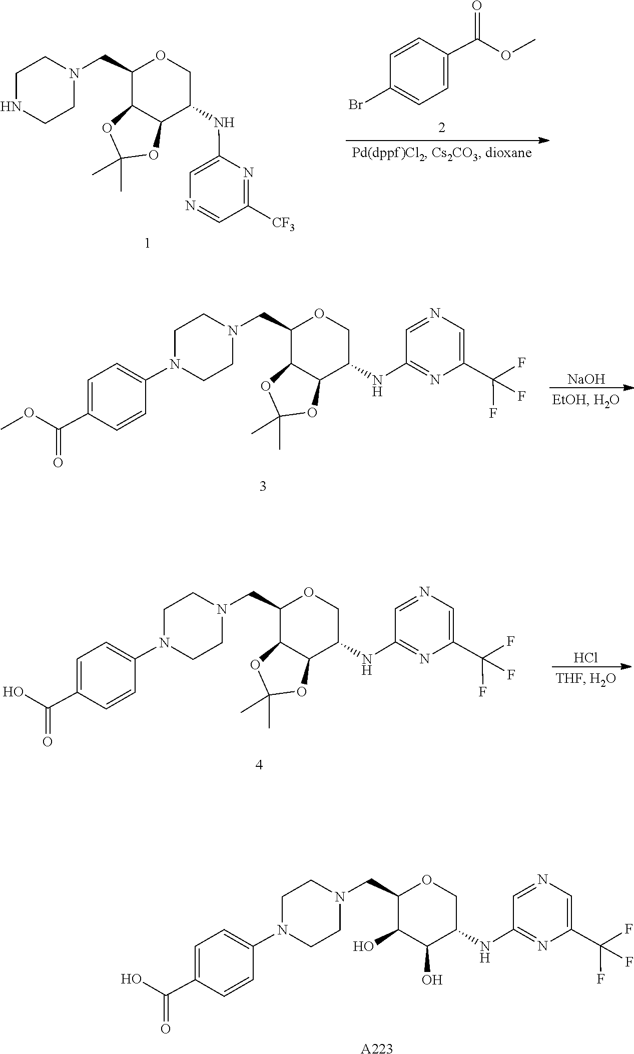

Step 1: To a solution of 1 (1.00 eq) and 2 (2.50 eq) in 1,4-Dioxane (3 mL) was added Pd(dppf)Cl₂ (0.20 eq) and Cs₂CO₃ (2.50 eq), the reaction mixture was stirred at 100° C. for 10 hours under nitrogen atmosphere. LCMS showed the desired mass was detected. The mixture was concentrated under reduced pressure. The residue was purified by flash (DCM/Methanol=20/1) to afford 3 as brown solid. LCMS found: [M+H]⁺=552.
Step 2: To a solution of 3 (1.00 eq) in EtOH (2 mL) and H₂O (0.5 mL) was added NaOH (5.00 eq), the mixture was stirred at 50° C. for 2 hours. The desired mass was observed by LCMS. The mixture was concentrated under reduced pressure to afford 4 (crude) as a colorless oil. LCMS found: [M+H]⁺=538.
Step 3: To a solution of 4 (1.00 eq) in THF (2 mL) was added HCl (1 mL, 6N) at 0° C., the reaction was stirred at 50° C. for 1 hour. LCMS showed the reaction was completed. The mixture was concentrated in vacuo. The residue was purified by reverse phase (C18, 5-17% ACN in water, 0.1% FA) to afford A223 (formate) as orange solid. Yield: 30.25%. LCMS found: [M+H]⁺=498. ¹H NMR (400 MHz, CD₃OD): δ 8.13 (brs, 1H, FA), 8.13 (s, 1H), 8.02 (s, 1H), 7.90 (d, J=9.0 Hz, 2H), 7.00 (d, J=9.0 Hz, 2H), 4.35 (td, J=10.5, 5.0 Hz, 1H), 4.18 (dd, J=11.0, 5.1 Hz, 1H), 3.91 (d, J=2.9 Hz, 1H), 3.80 (d, J=5.4 Hz, 1H), 3.70 (dd, J=10.6, 3.2 Hz, 1H), 3.54-3.43 (m, 4H) 3.21-3.12 (m, 2H), 3.10-3.01 (m, 4H), 2.98 (dd, J=13.4, 2.8 Hz, 1H).

### Preparation of (2R,3R,4S,5S)-2-(1-(piperidin-4-yl)-1H-1,2,3-triazol-4-yl)-5-((6-(trifluoromethyl)pyridin-2-yl)oxy)tetrahydro-2H-pyran-3,4-diol (A224)

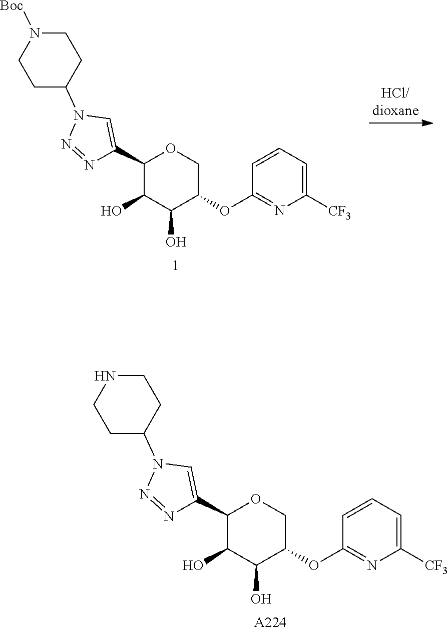

The solution of 1 (1.00 eq) in HCl/dioxane (2 mL) was stirred at 25° C. for 2 hours. LCMS showed the reaction was completed. The mixture was concentrated under reduced pressure. The residue was purified by prep-HPLC to give A224 as a white solid. Yield: 44.04%. LCMS found: [M+H]⁺=430. ¹H NMR (400 MHz, CD₃OD) δ 8.04 (s, 1H), 7.89 (t, J=8.0 Hz, 1H), 7.38 (d, J=7.6 Hz, 1H), 7.08 (d, J=8.4 Hz, 1H), 5.45 (td, J=10.0, 5.2 Hz, 1H), 4.90-4.87 (m, 1H), 4.82 (s, 1H) 4.44 (dd, J=10.8, 5.2 Hz, 1H), 4.17 (dd, J=2.8, 0.8 Hz, 1H), 4.05 (dd, J=10.0, 3.6 Hz, 1H), 3.56-3.49 (m, 2H), 3.42 (t, J=10.8 Hz, 1H), 3.20 (td, J=12.8, 2.8 Hz, 2H), 2.45-2.36 (m, 2H), 2.35-2.25 (m, 2H).

### Preparation of 4′-(((2R,3R,4R,5S)-3,4-dihydroxy-5-((6-(trifluoromethyl)pyridin-2-yl)amino)tetrahydro-2H-pyran-2-yl)methoxy)-[1,1′-biphenyl]-4-carboxylic acid (A225)

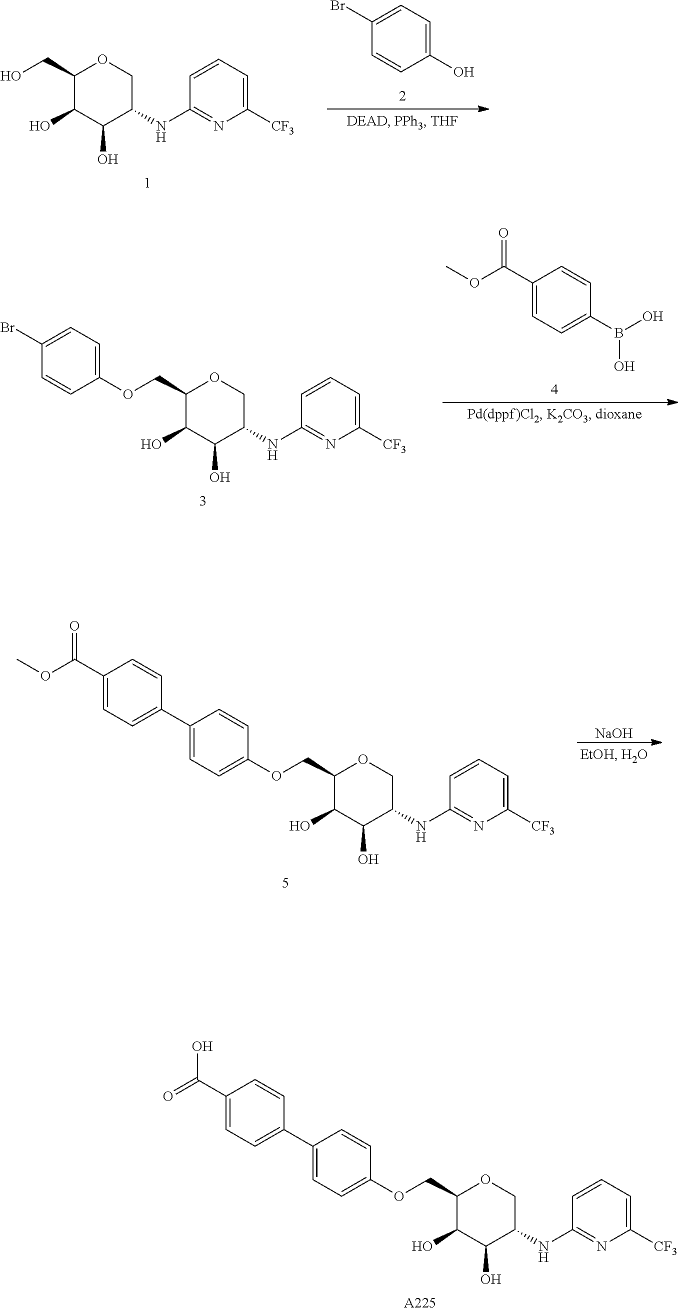

Step 1: To a solution of 1(1.00 eq), DEAD (3.00 eq) and PPh₃ (3.00 eq) in THE was added 2 (1.50 eq) at room temperature. The reaction mixture was stirred at 70° C. for 2 hours under N₂ atmosphere. LCMS showed the reaction was completed. The mixture was concentrated under reduced pressure. The residue was purified by silica gel chromatography (PE:EA=3:1) to give 3 as a white solid. Yield: 66.54%. LC-MS (ESI) found: [M+H]⁺=463.
Step 2: To a solution of 3 (1.00 eq) and 4 (1.50 eq) in dioxane was added K₂CO₃ (3.00 eq) and Pd(dppf)C12 (0.20 eq), the reaction mixture was stirred at 80° C. for 3 hours under N₂ atmosphere. LCMS showed the reaction was completed. The mixture was concentrated under reduced pressure, the residue was purified by silica gel chromatography (DCM/MeOH=50/1 to 15/1) to give 5 as a white solid. Yield: 33.95%. LC-MS (ESI) found: [M+H]⁺=519.
Step 3: To a solution of 5 (1.00 eq) in EtOH was added NaOH (1 mL, 10% in water), the mixture was stirred at 80° C. for 1 hour. LCMS showed the reaction was completed. The pH of the mixture was adjusted to 5 by HCl (1N), the mixture was concentrated under reduced pressure, the residue was purified by prep-HPLC to afford A225 as white solid. Yield: 29.43%. LC-MS (ESI) found: [M+H]⁺=505. ¹H NMR (400 MHz, CD₃OD) δ 8.06 (d, J=8.5 Hz, 2H), 7.69 (d, J=8.5 Hz, 2H) 7.64 (d, J=8.8 Hz, 2H), 7.53 (t, J=8.0 Hz, 1H), 7.07 (d, J=8.8 Hz, 2H), 6.89 (d, J=7.6 Hz, 1H), 6.74 (d, J=8.4 Hz, 1H), 4.39-4.29 (m, 1H), 4.27-4.18 (m, 3H), 4.04 (d, J=2.8 Hz, 1H) 3.84 (t, J=6.0 Hz, 1H), 3.68 (dd, J=10.4, 3.2 Hz, 1H), 3.19 (t, J=10.8 Hz, 1H).

### Preparation of (2R,3R,4R,5S)-2-((pyrimidin-2-yloxy)methyl)-5-((6-(trifluoromethyl) pyridine-2-yl)amino)tetrahydro-2H-pyran-3,4-diol (A226)

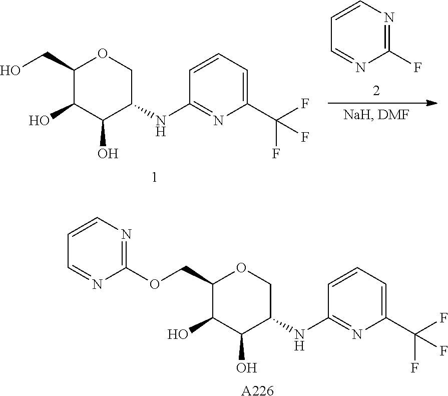

To a solution of 1 (1.00 eq) and 2 (0.90 eq) in DMF (2 mL) was added NaH (4.00 eq, 60% purity) at 0° C., the mixture was stirred at 0° C. for 2 hours. LCMS showed the desired mass was observed. The mixture was quenched with water, then concentrated in vacuo. The residue was purified by prep-HPLC to afford A226 as a brown solid. Yield: 11.2%. LCMS found: [M+H]⁺=387. ¹H NMR (400 MHz, CD₃OD): δ 8.58 (d, J=4.8 Hz, 2H), 7.53 (t, J=8.0 Hz, 1H), 7.11 (t, J=4.8 Hz, 1H) 6.89 (d, J=7.6 Hz, 1H), 6.73 (d, J=8.4 Hz, 1H), 4.62-4.52 (m, 2H), 4.33 (td, J=10.8, 5.2 Hz, 1H), 4.20 (dd, J=11.2, 5.2 Hz, 1H), 4.02 (d, J=2.8 Hz, 1H), 3.86 (ddd, J=6.4, 4.8, 0.8 Hz, 1H) 3.66 (dd, J=10.8, 3.2 Hz, 1H), 3.16 (t, J=10.8 Hz, 1H).

### Preparation of (2R,3R,4R,5S)-2-((pyridazin-3-yloxy)methyl)-5-((6-(trifluoromethyl) pyridine-2-yl)amino)tetrahydro-2H-pyran-3,4-diol (A227)

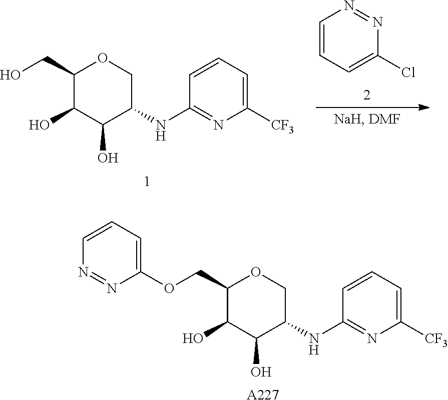

To a stirred solution of 1 (1.00 eq) and 2 (1.00 eq) in DMF was added NaH (4.00 eq, 60% purity) at 0° C., the mixture was stirred at 25° C. for 6 hours. LCMS showed one peak with desired mass was detected. The mixture was quenched with water, then concentrated under reduced pressure. The residue was purified by prep-HPLC to afford A227 as a white solid. Yield: 3.99%. LCMS found: [M+H]⁺=387. ¹H NMR (400 MHz, CD₃OD) δ 8.81 (dd, J=4.4, 1.2 Hz, 1H), 7.61 (dd, J=8.8, 4.4 Hz, 1H), 7.53 (t, J=8.0 Hz, 1H), 7.22 (dd, J=8.8, 1.2 Hz, 1H), 6.89 (d, J=7.6 Hz, 1H), 6.73 (d, J=8.4 Hz, 1H), 4.68 (dd, J=11.2, 4.8 Hz, 1H), 4.63 (dd, J=11.2, 7.2 Hz, 1H), 4.33 (td, J=10.8, 5.2 Hz, 1H), 4.22 (dd, J=11.2, 5.2 Hz, 1H), 4.02 (d, J=2.8 Hz, 1H), 3.90 (ddd, J=7.2, 4.8, 0.8 Hz, 1H), 3.67 (dd, J=10.4, 3.2 Hz, 1H), 3.16 (t, J=10.8 Hz, 1H).

### Preparation of 4′-(((2R,3R,4S,5S)-3,4-dihydroxy-5-((6-(trifluoromethyl)pyridin-2-yl)oxy)tetrahydro-2H-pyran-2-yl)methoxy)-[1,1′-biphenyl]-4-carboxylic acid (A228)

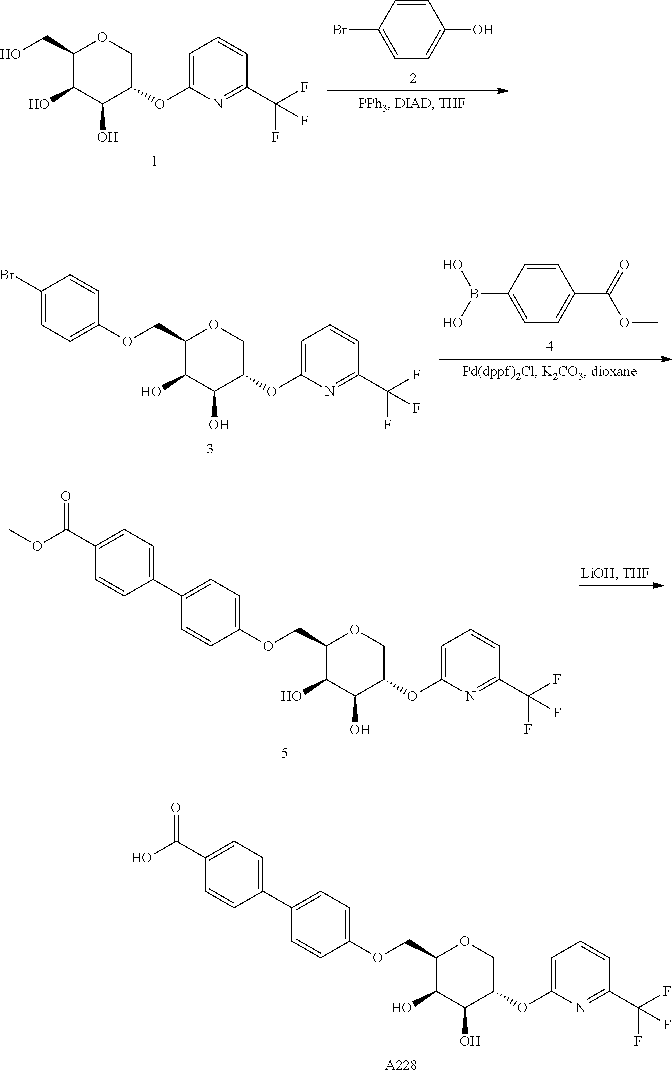

Step 1: To a solution of 1 (1.00 eq), 2 (1.20 eq) and PPh₃ (4.00 eq) in THF (3 mL) was added DIAD (4.00 eq) at 0° C. The mixture was stirred at 60° C. for 3 hours, then concentrated. The residue was purified by reverse phase (C18, 5-70% MeCN in water, 0.1% TFA) to afford 3 as white solid. Yield: 20%. LC-MS (ESI) found: 464 [M+H]⁺.
Step 2: A solution of 3 (1.00 eq) and 4 (1.20 eq), K₂CO₃ (2.00 eq) and Pd(dppf)C1 (0.10 eq) in dioxane (5 mL) and H₂O (1 mL) was stirred at 90° C. for 10 hours. The mixture was concentrated and the residue was purified by flash chromatography (silica gel, 0˜10% MeOH in DCM) to give 5 as a white solid. Yield: 29.8%. LC-MS (ESI) found: 520 [M+H]⁺.
Step 3: A solution of 5 (1.00 eq) and LiOH (2.00 eq) in THF (5 mL) and H₂O (1 mL) was stirred at 25° C. for 10 hours. The mixture was concentrated and the residue was purified by reverse phase (C18, 5-55% MeCN in water) to afford A228 as white solid. Yield: 78.9%. LC-MS (ESI) found: 506 [M+H]⁺. ¹H NMR (400 MHz, CD₃OD): δ 8.06 (d, J=8.5 Hz, 2H), 7.87 (t, J=8.0 Hz, 1H), 7.75-7.55 (m, 4H), 7.37 (d, J=7.3 Hz, 1H), 7.13-7.03 (m, 3H), 5.41 (td, J=9.9, 5.4 Hz, 1H), 4.39 (dd, J=10.8, 5.4 Hz, 1H), 4.28-4.19 (m, 2H), 4.12 (d, J=2.6 Hz, 1H), 3.91 (dd, J=9.7, 3.5 Hz, 2H), 3.34 (s, 1H).

### Preparation of (2R,3R,4S,5S)-2-((pyrazin-2-yloxy)methyl)-5-((6-(trifluoromethyl)pyridin-2-yl)oxy)tetrahydro-2H-pyran-3,4-diol (A229)

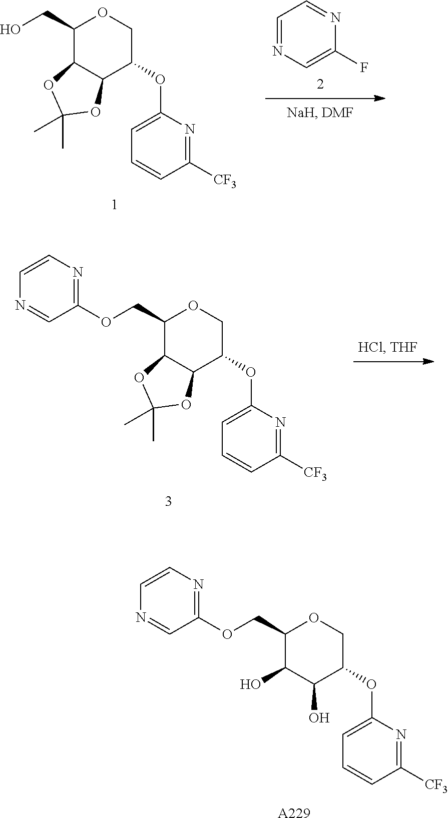 [SMILES: CC1(C)O[C@@H]2[C@H](O1)[C@@H](Oc1cccc(C(F)(F)F)n1)CO[C@@H]2CO ; Fc1cnccn1 ; CC1(C)O[C@@H]2[C@H](O1)[C@@H](Oc1cccc(C(F)(F)F)n1)CO[C@@H]2COc1cnccn1 ; O[C@@H]1[C@H](O)[C@@H](Oc2cccc(C(F)(F)F)n2)CO[C@@H]1COc1cnccn1 ; "A229"]

Step 1: To a solution of 1 (1.00 eq) and 2 (1.10 eq) in DMF (3 mL) was added NaH (2.00 eq) at 0° C. The mixture was stirred at 25° C. for 3 hours, then quenched with NH₄Cl aq. The residue was purified by reverse phase (C18, 5-70% MeCN in water, 0.1% TFA) to afford 3 as white solid. Yield: 72.7%. LC-MS (ESI) found: 428 [M+H]⁺.
Step 2: A solution of 3 (1.00 eq) and HCl (2 mL, 2 N in H₂O) in THF (5 mL) was stirred at 25° C. for 10 hours. The mixture was concentrated and the residue was purified by reverse phase (C18, 5-55% MeCN in water) to afford A229 as white solid. Yield: 85.5%. LC-MS (ESI) found: 388 [M+H]⁺. ¹H NMR (400 MHz, CD₃OD): δ 8.17 (ddd, J=20.4, 19.7, 2.0 Hz, 3H), 7.87 (t, J=7.9 Hz, 1H), 7.36 (d, J=7.3 Hz, 1H), 7.05 (d, J=8.4 Hz, 1H), 5.39 (td, J=10.0, 5.4 Hz, 1H), 4.61-4.44 (m, 2H), 4.37 (dd, J=10.8, 5.4 Hz, 1H), 4.08 (dd, J=3.4, 1.0 Hz, 1H), 3.97-3.83 (m, 2H) 3.29-3.23 (m, 1H).

### Preparation of tert-butyl 3-(((2R,3R,4R,5S)-3,4-dihydroxy-5-((6-(trifluoromethyl) pyrazin-2-yl) amino) tetrahydro-2H-pyran-2-yl) methyl)-3,6-diazabicyclo [3.2.0] heptane-6-carboxylate (A230)

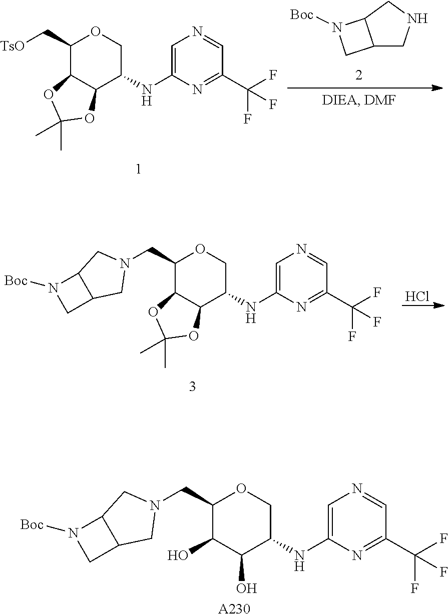

Step 1: To a solution of 1 (1.00 eq) in DMF (1.2 mL) was added DIEA (3.50 eq) and 2 (3.00 eq), the mixture was stirred at 100° C. for 16 hours. After completion of the reaction, the mixture was diluted with brine and extracted with EA. The combined organic layer was dried over anhydrous sodium sulfate, filtered and concentrated. The residue was purified by silica gel chromatography (DCM/Methanol=10/1) to give 3 as a brown solid. Chemical Formula: C₂₄H₃₄F₃N₅O₅, LCMS found: [M+H]⁺=530.
Step 2: To a solution of 3 (1.00 eq) in THF (5.5 mL) was added 0.5N HCl (1.00 eq), the reaction mixture was stirred at 25° C. for 48 hours, the pH of the mixture was adjusted to 7 by NH₃·H₂O, and then the mixture was concentrated under reduced pressure. The residue was purified by pre-HPLC to give A230 (yield: 46.75%) as white solid. Chemical Formula: C₂₁H₃₀F₃N₅O₅, LCMS found: [M+H]⁺=490. ¹H NMR (400 MHz, CD₃OD): δ 8.11 (s, 1H), 8.00 (s, 1H), 4.59 (dd, J=6.4, 3.9 Hz, 1H), 4.40-4.31 (m, 1H), 4.13 (dd, J=10.8, 4.9 Hz, 1H), 4.02-3.90 (m, 2H), 3.69-3.56 (m, 3H), 3.43 (d, J=10.4 Hz, 1H), 3.31-3.09 (m, 2H), 3.01-2.89 (m, 2H), 2.85-2.76 (m, 1H), 2.27-2.20 (m, 1H), 2.11-2.04 (m, 1H), 1.44 (s, 9H).

### Preparation of (2R,3R,4R,5S)-2-(isoindolin-2-ylmethyl)-5-((6-(trifluoromethyl)pyrazin-2-yl)amino)tetrahydro-2H-pyran-3,4-diol (A231)

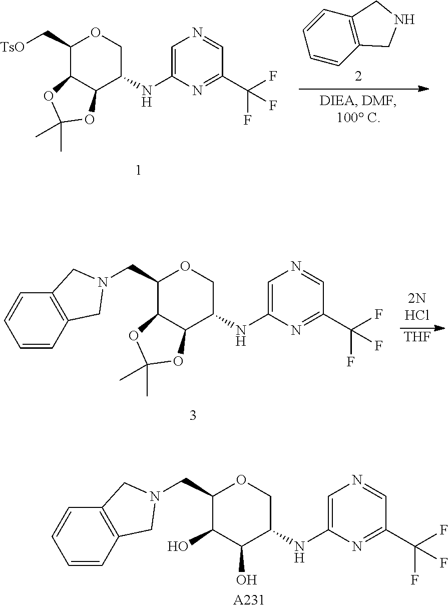

Step 1: The mixture of 1 (1.00 eq), 2 (1.50 eq) and DIEA (3.00 eq) in DMF was stirred at 100° C. for 16 hours. The mixture was diluted with EA and washed with water and brine. The organic layer was concentrated to give 3 (crude) as a dark red solid. LCMS found: [M+H]⁺=451.
Step 2: The mixture of 3 (1.00 eq) and 2N HCl (30.00 eq) in THE was stirred at 20° C. for 16 hours. The mixture was concentrated in vacuum, the residue was purified by Prep-HPLC to give A231 as a yellow solid. Yield: 39.5%. LCMS found: [M+H]⁺=411.

¹H NMR (400 MHz, CD₃OD) δ 8.13 (s, 1H), 8.02 (s, 1H), 7.31 (s, 4H), 4.40 (s, 4H), 4.35 (dd, J=10.4, 5.2 Hz, 1H), 4.21 (dd, J=11.2, 5.2 Hz, 1H), 3.92 (d, J=2.4 Hz, 1H), 3.80 (d, J=6.0 Hz, 1H), 3.71 (dd, J=10.4, 3.2 Hz, 1H), 3.55-3.46 (m, 1H), 3.29-3.26 (m, 1H), 3.19 (t, J=10.8 Hz, 1H).

### Preparation of (2R,3R,4R,5S)-2-((3,4-dihydroisoquinolin-2(1H)-yl)methyl)-5-((6-(trifluoromethyl)pyrazin-2-yl)amino)tetrahydro-2H-pyran-3,4-diol (A232)

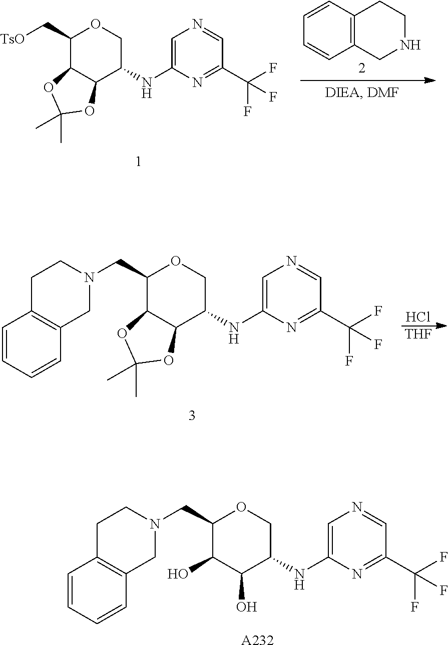

Step 1: To a solution of 1 (1.00 eq) in DMF was added DIEA (3.00 eq) and 2 (1.00 eq), the reaction mixture was stirred at 100° C. for 16 hours. LCMS showed the reaction was completed. The mixture was diluted with ethyl acetate, washed with water and brine, the combined organic layers were dried over anhydrous sodium sulfate, filtered and concentrated under reduced pressure to give 3 (crude) as yellow oil. LC-MS (ESI) found: [M+H]⁺=465.
Step 2: To a solution of 3 (1.00 eq) in THE was added HCl (1 mL, 2 N) at 25° C. The reaction mixture was stirred at 25° C. for 2 hours. LCMS showed the reaction was completed. The mixture was concentrated under reduced pressure and the residue was purified by prep-HPLC (FA condition) to afford A232 as white solid. Yield: 70.70%. LC-MS (ESI) found: [M+H]⁺=425. ¹H NMR (400 MHz, CD₃OD) δ 8.13 (s, 1H), 8.01 (s, 1H), 7.26-7.18 (m, 3H), 7.15 (d, J=7.2 Hz, 1H), 4.36 (td, J=10, 5.2 Hz, 1H), 4.25 (s, 2H), 4.19 (dd, J=11.2, 5.2 Hz, 1H), 3.96-3.90 (m, 2H), 3.72 (dd, J=10.4, 3.2 Hz, 1H), 3.42-3.37 (m, 3H), 3.25-3.19 (m, 2H), 3.11 (t, J=6.4 Hz, 2H).

### Preparation of (2R,3R,4S,5S)-2-(piperidin-1-ylmethyl)-5-((6-(trifluoromethyl)pyridin-2-yl)oxy)tetrahydro-2H-pyran-3,4-diol (A233)

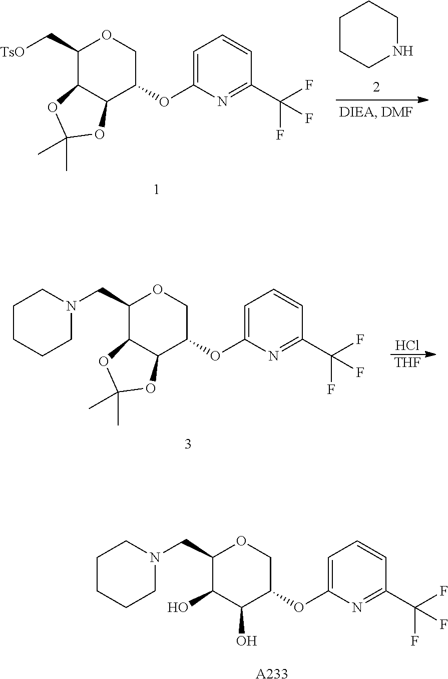

Step 1: To a solution of 1 (1.00 eq) in DMF was added DIEA (3.00 eq) and 2 (1.00 eq), the reaction mixture was stirred at 100° C. for 16 hours. LCMS showed the reaction was completed. The mixture was diluted with ethyl acetate, washed with water and brine, the combined organic layers were dried over anhydrous sodium sulfate, filtered and concentrated under reduced pressure to give 3 (crude) as yellow oil. LC-MS (ESI) found: [M+H]⁺=417.
Step 2: To a solution of 3 (1.00 eq) in THE was added HCl (1 mL, 2 N) at 25° C. The reaction mixture was stirred at 25° C. for 2 hours. LCMS showed the reaction was completed. The mixture was concentrated under reduced pressure and the residue was purified by prep-HPLC to afford A233 as white solid. Yield: 60.32%. LC-MS (ESI) found: [M+H]⁺=377. ¹H NMR (400 MHz, CD₃OD) δ 7.86 (t, J=8.0 Hz, 1H), 7.35 (d, J=7.6 Hz, 1H), 7.04 (d, J=8.4 Hz, 1H), 5.33 (td, J=10.0, 5.2 Hz, 1H), 4.32 (dd, J=10.8, 5.6 Hz, 1H), 3.91 (dd, J=3.2, 0.8 Hz, 1H), 3.82 (dd, J=10.0, 3.2 Hz, 1H), 3.65 (dd, J=7.2, 2.4 Hz, 1H), 3.20 (t, J=10.4 Hz, 1H), 2.78 (dd, J=13.6, 7.2 Hz, 1H), 2.66-2.48 (m, 5H), 1.66-1.57 (m, 4H), 1.53-1.43 (m, 2H).

### Preparation of (2R,3R,4S,5S)-2-(phenoxymethyl)-5-((6-(trifluoromethyl)pyridin-2-yl)oxy)tetrahydro-2H-pyran-3,4-diol (A234)

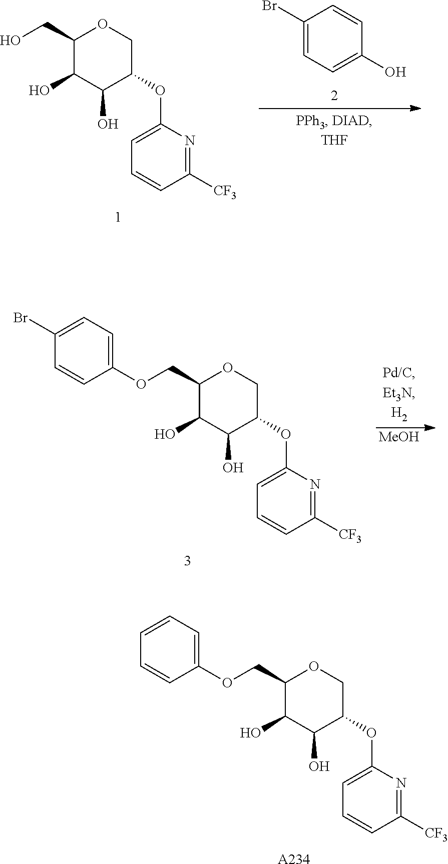

Step 1: To a solution of 1 (1.00 eq), 2 (1.20 eq) and PPh3 (3.00 eq) in THF (20 mL) was added DIAD (3.00 eq) at 0° C. The reaction was stirred at 60° C. for 2 hours under nitrogen atmosphere. LCMS showed the desired mass was detected. The resulting mixture was filtered and the filtrate was concentrated in vacuo to give crude 3 as a colorless oil. LC-MS (ESI) found: 464 [M+H]⁺.
Step 2: To a solution of 3 (1.00 eq) in MeOH (10 mL) was added Et₃N (3.00 eq) and Pd/C (0.10 eq), the mixture was stirred at 25° C. for 3 hours under hydrogen atmosphere (15 psi). LCMS showed the desired mass was detected. The mixture was filtered, the filtrate was concentrated in vacuum. The residue was purified by prep-HPLC to afford A234 a white solid. Yield: 52.3%. LC-MS (ESI) found: 386 [M+H]⁺. 1H NMR (400 MHz, CD₃OD) δ 7.87 (t, J=7.9 Hz, 1H), 7.36 (d, J=7.3 Hz, 1H), 7.30-7.21 (m, 2H), 7.05 (d, J=8.4 Hz, 1H), 6.97-6.90 (m, 3H), 5.39 (td, J=10.0, 5.4 Hz, 1H), 4.38 (dd, J=10.8, 5.5 Hz, 1H), 4.17 (dd, J=6.0, 1.9 Hz, 2H), 4.10 (d, J=2.6 Hz, 1H), 3.88 (dt, J=9.8, 4.9 Hz, 2H), 3.27 (t, J=10.8 Hz, 1H).

### Preparation of (2R,3R,4S,5S)-2-((pyrimidin-2-yloxy)methyl)-5-((6-(trifluoromethyl)pyridin-2-yl)oxy)tetrahydro-2H-pyran-3,4-diol (A235)

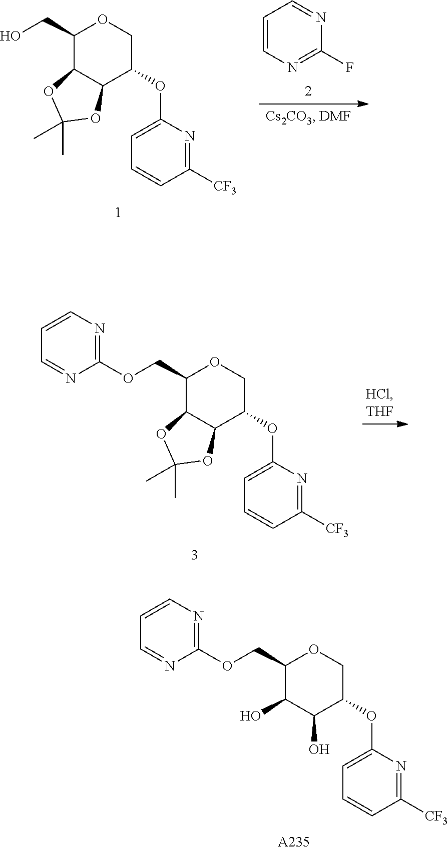 [SMILES: CC1(C)O[C@@H]2[C@H](O1)[C@@H](Oc1cccc(C(F)(F)F)n1)CO[C@@H]2CO ; Fc1ncccn1 ; CC1(C)O[C@@H]2[C@H](O1)[C@@H](Oc1cccc(C(F)(F)F)n1)CO[C@@H]2COc1ncccn1 ; O[C@@H]1[C@H](O)[C@@H](Oc2cccc(C(F)(F)F)n2)CO[C@@H]1COc1ncccn1 ; "A235"]

Step 1: To a solution of 1 (1.00 eq) and 2 (1.20 eq) in DMF (10 mL) was added Cs₂CO₃ (2.00 eq), the mixture was stirred at 60° C. for 2 hours. LCMS showed the desired mass was detected. The resulting mixture was filtered and the filtrate was concentrated in vacuo to give crude 3 as a colorless oil. LC-MS (ESI) found: 428 [M+H]⁺.
Step 2: The solution of 3 (1.00 eq) and HCl (1 mL, 2N) in THF (2 mL) was stirred at 25° C. for 2 hours. LCMS showed the desired mass was detected. The mixture was concentrated under reduced pressure, the residue was purified by prep-HPLC to give A235 as a white solid. Yield: 63.46%. LCMS found: [M+H]⁺=388. ¹H NMR (400 MHz, CD₃OD) δ 8.57 (d, J=4.8 Hz, 2H), 7.87 (t, J=8.0 Hz, 1H), 7.36 (d, J=7.6 Hz, 1H), 7.11 (t, J=4.8 Hz, 1H), 7.05 (d, J=8.4 Hz, 1H), 5.39 (td, J=10.0, 5.2 Hz, 1H), 4.57 (qd, J=11.2, 6.1 Hz, 2H), 4.36 (dd, J=10.8, 5.4 Hz, 1H), 4.09 (d, J=4.3 Hz, 1H), 3.95-3.84 (m, 2H), 3.26 (t, J=10.8 Hz, 1H). 1H).

### Preparation of (2R,3R,4R,5S)-2-((4-bromophenoxy)methyl)-5-((6-(trifluoromethyl)pyridin-2-yl)amino)tetrahydro-2H-pyran-3,4-diol (A236)

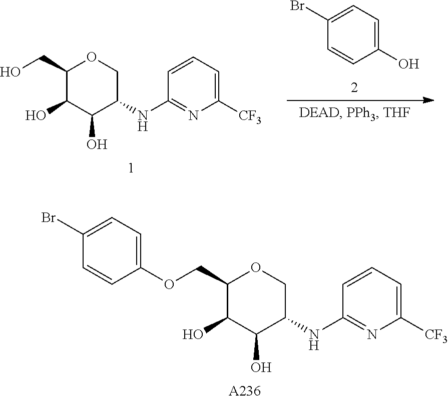

To a solution of 1 (1.00 eq), DEAD (3.00 eq) and PPh₃ (3.00 eq) in THE was added 2 (1.50 eq) at 25° C., the reaction mixture was stirred at 70° C. for 2 hours under N₂ atmosphere. LCMS showed the reaction was completed. The mixture was concentrated under reduced pressure. The residue was purified by silica gel chromatography (DCM/MeOH=50/1 to 10/1), the obtained crude product was purified by prep-HPLC to give A236 as a white solid. Yield: 66.54%. LC-MS (ESI) found: [M+H]⁺=463, 465. H NMR (400 MHz, DMSO) δ 7.55 (t, J=8.0 Hz, 1H), 7.45 (d, J=9.2 Hz, 2H), 6.97-6.86 (m, 4H), 6.77 (d, J=8.4 Hz, 1H), 4.86 (d, J=4.8 Hz, 1H), 4.80 (d, J=6.4 Hz, 1H), 4.13-4.02 (m, 3H), 3.98 (dd, J=10.8, 5.2 Hz, 1H), 3.86-3.80 (m, 1H), 3.75-3.67 (m, 1H), 3.54 (ddd, J=10.0, 6.4, 3.2 Hz, 1H), 2.97 (t, J=10.4 Hz, 1H).

We claim:

1. An ASGPR-binding extracellular protein degrader compound of the formula: 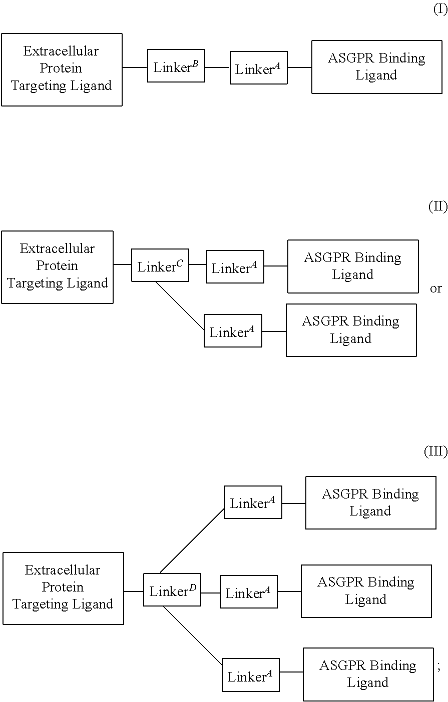
or a pharmaceutically acceptable salt thereof,
wherein the ASGPR Binding Ligand is selected from: 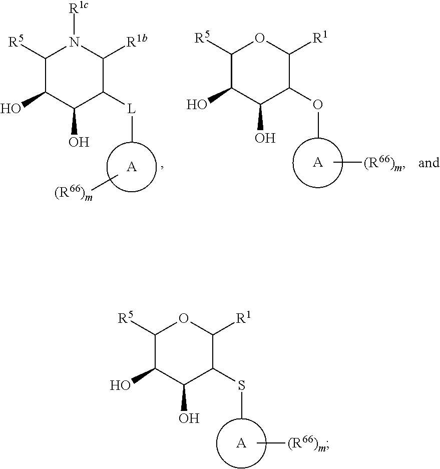
R¹, R^{1b}, and R⁵ are independently selected from hydrogen, C₀-C₆alkyl-cyano, alkyl, alkenyl, alkynyl, haloalkyl, aryl, arylalkyl, heteroaryl, heteroarylalkyl, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, and C₀-C₆alkylN₃, each of which except hydrogen, F, Cl, and Br is optionally substituted with 1, 2, or 3 substituents independently selected at each occurrence from R⁹⁹;
R^{1c} is selected from hydrogen, alkyl, alkenyl, alkynyl, haloalkyl, —C(O)R³, —S(O)R³, —C(S)R³, and —S(O)₂R³;
wherein one of R¹, R^{1b}, R^{1c}, and R⁵ is replaced with a bond to Linker^{A};
L is selected from 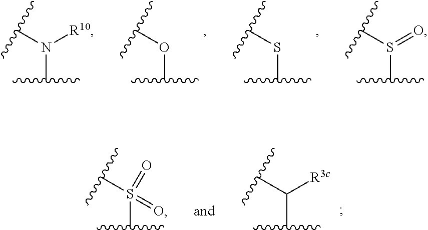
R³ and R^{3c} are independently selected at each occurrence from hydrogen, alkyl, haloalkyl, arylalkyl, heteroarylalkyl, alkenyl, alkynyl, aryl, heteroaryl, heterocycle, —OR⁸, and —NR⁸R⁹;
R⁶ and R⁷ are independently selected at each occurrence from hydrogen, alkyl, arylalkyl, heteroarylalkyl, alkenyl, alkynyl, aryl, haloalkyl, heteroaryl, heterocycle, -alkyl-OR⁸, -alkyl-NR⁸R⁹, C(O)R³, S(O)R³, C(S)R³, and S(O)₂R³;
R⁸ and R⁹ are independently selected at each occurrence from hydrogen, alkyl, arylalkyl, heteroarylalkyl, alkenyl, alkynyl, aryl, heteroaryl, and heterocycle;
R¹⁰ is selected from hydrogen, alkyl, haloalkyl, arylalkyl, heteroarylalkyl, alkenyl, alkynyl, aryl, heteroaryl, heterocycle, C(O)R³, S(O)R³, C(S)R³, and S(O)₂R³;
R⁶⁶ is independently selected at each instance from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰;
m is independently 0, 1, 2, or 3, as allowed by valence;
n is independently selected at each instance from 0, 1, 2, or 3;
\textcircled{A} is heteroaryl;
R⁹⁹ and R¹⁰⁰ are independently selected at each instance from alkyl, alkenyl, alkynyl, haloalkyl, —OR⁶, F, Cl, Br, I, —NR⁶R⁷, —NR⁸R⁹, heterocycle, heteroaryl, aryl, cyano, nitro, hydroxyl, azide, amide, —SR³, —S(O)(NR⁶)R³, —NR⁸C(O)R³, —C(O)NR⁶R⁷, —C(O)OR³, —C(O)R³, and —SF₅;
Linker^{A} and Linker^{B} are: 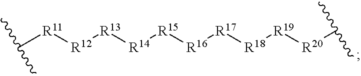
wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, —CH₂CH₂—[O—(CH₂)₂]ₙ—O—, —CH₂CH₂—[O—(CH₂)₂]ₙ—NR⁶—, —CH₂CH₂—[O—(CH₂)₂]ₙ—, —[—(CH₂)₂—O—]ₙ—, —[O—(CH₂)₂]ₙ—, —[O—CH(CH₃)C(O)]ₙ—, —[C(O)—CH(CH₃)—O]ₙ—, —[O—CH₂C(O)]ₙ—, —[C(O)—CH₂—O]ₙ—, a divalent residue of a fatty acid, a divalent residue of an unsaturated or saturated mono- or di-carboxylic acid, each of which is optionally substituted with 1, 2, or 3 substituents independently selected from R²¹;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, heterocyclyl, —SR³, —C(O)OR³, —OR³, 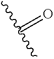 [SMILES: *=O]
and heterocycle;
Linker^{C} is: 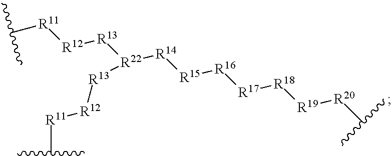
wherein:
R²² is independently at each occurrence selected from the group consisting of alkyl, —C(O)N—, —NC(O)—, —N—, —C(R²¹)—, —P(O)O—, —P(O)—, —P(O)(NR⁶R⁷)N—, alkenyl, haloalkyl, aryl, heterocycle, and heteroaryl, each of which is optionally substituted with 1, 2, or 3 substituents independently selected from R²¹;
Linker^{D} is selected from: 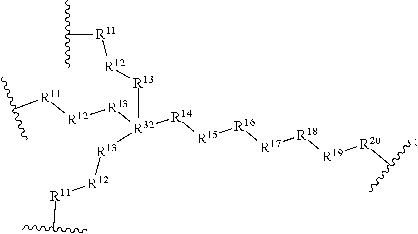
wherein:
R³² is independently at each occurrence selected from the group consisting of alkyl, N⁺X⁻, —C—, alkenyl, haloalkyl, aryl, heterocycle, and heteroaryl, each of which is optionally substituted with 1, 2, or 3 substituents independently selected from R²¹;
X⁻ is Br⁻ or Cl⁻; and
Extracellular Protein Targeting Ligand is a means for binding a targeted extracellular protein that modulates a disease in a human.

2. The compound of claim 1, wherein L is selected from the group consisting of

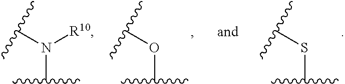

3. The compound of claim 1, wherein L is

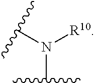

4. The compound of claim 1, wherein L is

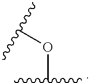 [SMILES: *O*]

5. The compound of claim 1, wherein the ASPGR Binding Ligand is

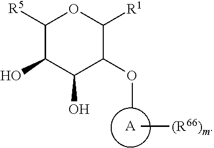

6. The compound of claim 1, wherein the ASPGR Binding Ligand is

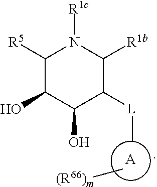

7. The compound of claim 1, wherein the ASPGR Binding Ligand is

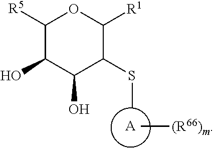

8. The compound of claim 1, wherein 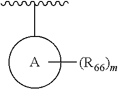
is selected from: 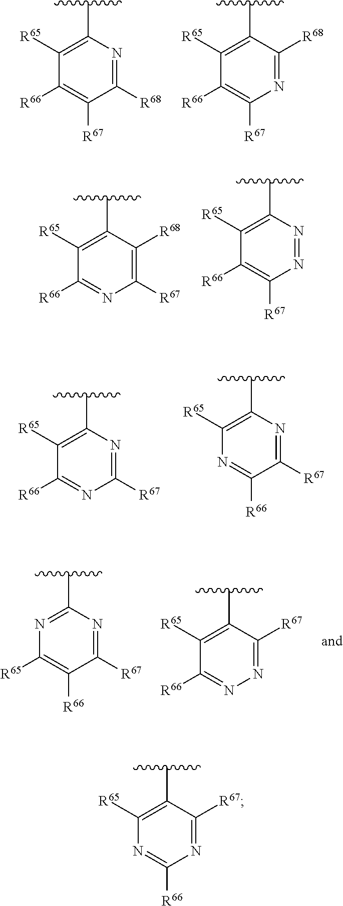
wherein
R⁶⁵, R⁶⁷, and R⁶⁸ are independently selected from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰.

9. The compound of claim 1, wherein 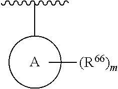
is selected from: 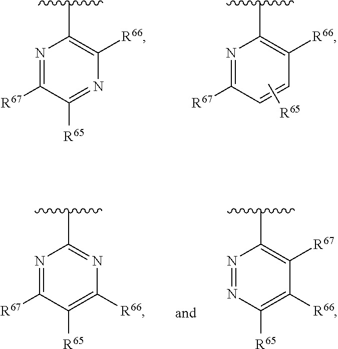
wherein
R⁶⁵, R⁶⁷, and R⁶⁸ are independently selected from hydrogen, C₀-C₆alkyl-cyano, alkyl, alkenyl, alkynyl, haloalkyl, F, Cl, Br, I, heterocycle, heterocycloalkyl, haloalkoxy, C₀-C₆alkyl-OR⁶, C₀-C₆alkyl-SR⁶, C₀-C₆alkyl-NR⁶R⁷, C₀-C₆alkyl-C(O)R³, C₀-C₆alkyl-S(O)R³, C₀-C₆alkyl-C(S)R³, C₀-C₆alkyl-S(O)₂R³, —N═S(O)(R³)₂, and C₀-C₆alkylN₃, each of which is optionally substituted with 1, 2, or 3 substituents independently selected at each occurrence from R¹⁰⁰.

10. The compound of claim 8, wherein R⁶⁷ is haloalkyl.

11. The compound of claim 8, wherein R⁶⁷ is trifluoromethyl.

12. The compound of claim 10, wherein is 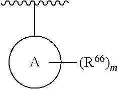
is

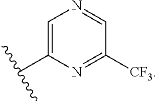 [SMILES: *c1cncc(C(F)(F)F)n1]

13. The compound of claim 10, wherein is 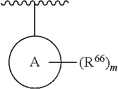
is

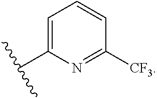 [SMILES: *c1cccc(C(F)(F)F)n1]

14. The compound of claim 1, wherein the ASGPR Binding Ligand is:

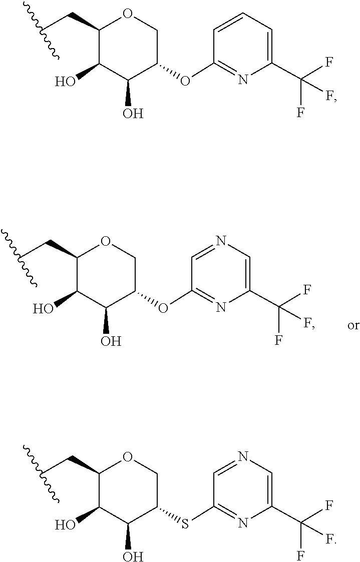 [SMILES: *C[C@H]1OC[C@H](Oc2cccc(C(F)(F)F)n2)[C@@H](O)[C@H]1O ; *C[C@H]1OC[C@H](Oc2cncc(C(F)(F)F)n2)[C@@H](O)[C@H]1O ; *C[C@H]1OC[C@H](Sc2cncc(C(F)(F)F)n2)[C@@H](O)[C@H]1O]

15. The compound of claim 1, wherein the ASPGR Binding Ligand is:

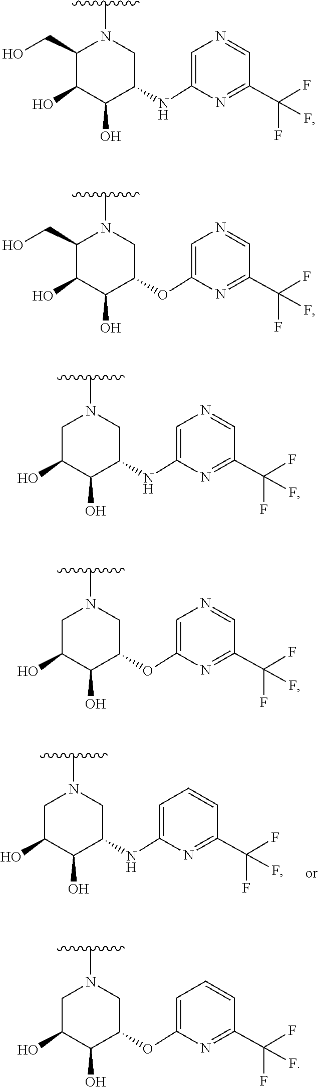 [SMILES: *N1C[C@H](Nc2cncc(C(F)(F)F)n2)[C@@H](O)[C@@H](O)[C@H]1CO ; *N1C[C@H](Oc2cncc(C(F)(F)F)n2)[C@@H](O)[C@@H](O)[C@H]1CO ; *N1C[C@H](Nc2cncc(C(F)(F)F)n2)[C@@H](O)[C@@H](O)C1 ; *N1C[C@H](Oc2cncc(C(F)(F)F)n2)[C@@H](O)[C@@H](O)C1 ; *N1C[C@H](Nc2cccc(C(F)(F)F)n2)[C@@H](O)[C@@H](O)C1 ; *N1C[C@H](Oc2cccc(C(F)(F)F)n2)[C@@H](O)[C@@H](O)C1]

16. The compound of claim 1, wherein the Extracellular Protein Targeting Ligand binds IgG.

17. The compound of claim 16, wherein the IgG targeting Extracellular Protein Targeting Ligand is Fc-BP-2.

18. The compound of claim 16, wherein the IgG targeting Extracellular Protein Targeting Ligand is Fc-III.

19. The compound of claim 16, wherein the Extracellular Protein Targeting Ligand binds autoantibodies to the R 1 adrenergic receptor.

20. The compound of claim 1, wherein the Extracellular Protein Targeting Ligand binds IgM.

21. The compound of claim 1, wherein the Extracellular Protein Targeting Ligand binds IgE.

22. The compound of claim 1, wherein the Extracellular Protein Targeting Ligand binds IgA.

23. The compound of claim 1, wherein Linker^{A} is: 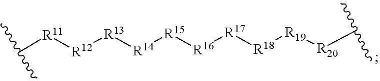
wherein:
R¹¹, R¹², R¹³, R¹⁴, R¹⁵, R¹⁶, R¹⁷, R¹⁸, R¹⁹, and R²⁰ are independently at each occurrence selected from the group consisting of a bond, alkyl, —C(O)—, —C(O)O—, —OC(O)—, —SO₂—, —S(O)—, —C(S)—, —C(O)NR⁶—, —NR⁶C(O)—, —O—, —S—, —NR⁶—, —C(R²¹R²¹)—, —P(O)(R³)O—, —P(O)(R³)—, a divalent residue of a natural or unnatural amino acid, alkenyl, alkynyl, haloalkyl, alkoxy, aryl, heterocycle, heteroaryl, each of which is optionally substituted with 1 substituent independently selected from R²¹;
R²¹ is independently at each occurrence selected from the group consisting of hydrogen, alkyl, alkenyl, alkynyl, F, Cl, Br, I, hydroxyl, alkoxy, azide, amino, cyano, —NR⁶R⁷, —NR⁸SO₂R³, —NR⁸S(O)R³, haloalkyl, aryl, heteroaryl, and heterocycle.

24. The compound of claim 22, wherein four of R¹¹, R¹², R¹³, R¹⁴, R¹⁵, R¹⁶, R¹⁷, R¹⁸, R¹⁹, and R²⁰ are bond.

25. The compound of claim 22, wherein three of R¹¹, R¹², R¹³, R¹⁴, R¹⁵, R¹⁶, R¹⁷, R¹⁸, R¹⁹, and R²⁰ are bond.

26. The compound of claim 22, wherein two of R¹¹, R¹², R¹³, R¹⁴, R¹⁵, R¹⁶, R¹⁷, R¹⁸, R¹⁹, and R²⁰ are bond.

27. The compound of claim 22, wherein Linker^{A} is selected from:

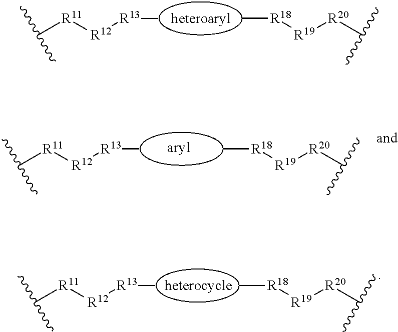

28. The compound of claim 1, wherein the compound is of Formula: 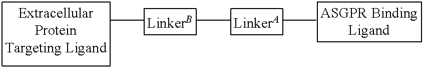
or a pharmaceutically acceptable salt thereof.

29. A pharmaceutical composition comprising a compound of claim 1 and a pharmaceutically acceptable carrier.

30. A compound selected from the group consisting of: 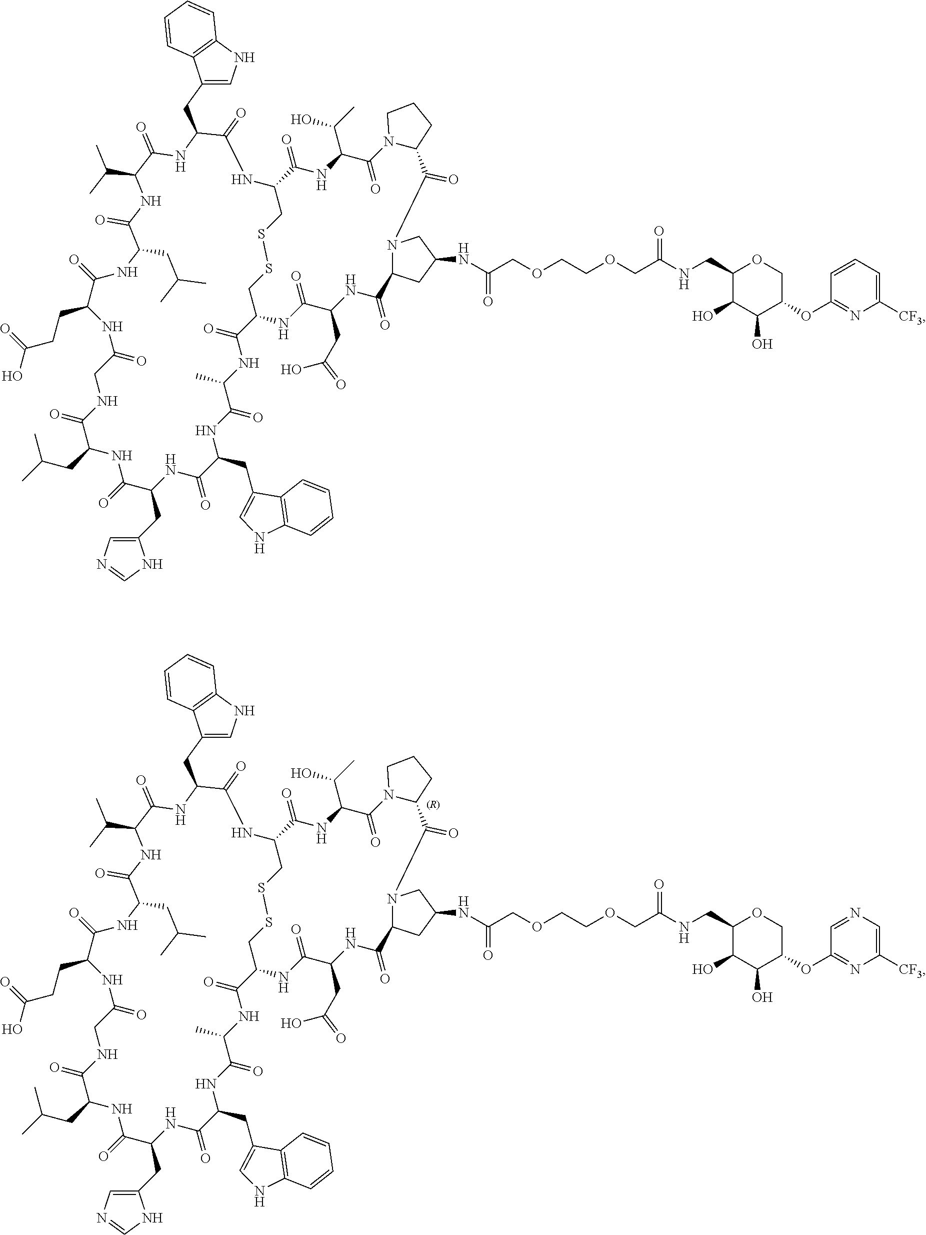 [chemical structure]
